(19) United States
(12) Patent Application Publication
(10) Pub. No.: US 2026/0062443 A1
(43) Pub. Date: Mar. 5, 2026

(54) PHARMACEUTICAL COMPOUNDS FOR THE TREATMENT OF COMPLEMENT MEDIATED DISORDERS

(71) Applicant: ALEXION PHARMACEUTICALS, INC., Boston, MA (US)

(72) Inventors: Venkat Rao GADHACHANDA, Hamden, CT (US); Evans O. ONYANGO, Hamden, CT (US); Qi ZHOU, Orange, CT (US)

(21) Appl. No.: 19/103,629

(22) PCT Filed: Aug. 18, 2023
(86) PCT No.: PCT/US2023/030545

§ 371 Date: Feb. 13, 2025

Related U.S. Application Data
(60) Provisional application No. 63/400,252, filed on Aug. 23, 2022.

(51) Int. Cl. C07K 5/097; A61K 38/00; C07K 5/08

(52) U.S. Cl. CPC C07K 5/0821; A61K 38/00; C07K 5/0827

(57) ABSTRACT

This disclosure provides peptide based compounds, compositions, and methods to treat medical disordeds, such as complement-mediated disorders, including complement C1s-mediated disorders, such as acute antibody-mediated rejection, amyotrophic lateral sclerosis, autoimmune blistering disease, bullous pemphigoid, geographic atrophy, or Guillain-Barre Syndrome, comprising administering to a subject in need thereof a therapeutically effective amount of the compound.

### FIELD OF THE DISCLOSURE

Herein are provided pharmaceutical compounds to treat medical disorders, such as complement-mediated disorders, including complement C1-mediated disorders.

### BACKGROUND OF THE DISCLOSURE

The complement system is a part of the innate immune system which does not adapt to changes over the course of the subject's life but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells), and agglutination (clustering and binding of pathogens together).

The complement system has three pathways: classical, alternative, and lectin. The classical pathway is triggered by antibody-antigen complexes with the antibody isotypes IgG and IgM. The antibody-antigen complex binds to C1 and this initiates the cleavage of C4 and C2 to generate C3 convertase that then splits C3 into C3a and C3b. C3a interacts with its C3a receptor to recruit leukocytes, while C3b binds to C3 convertase to form C5 convertase. C5 convertase cleaves C5 into C5a and C5b. Similar to C3a, C5a interacts with its C5a receptor to recruit leukocytes, but C5b interacts with C6, C7, C8, and C8 and together these proteins form the cylindrical membrane attack complex (MAC) that causes the cell to swell and burst. These immune responses can be inhibited by preventing C1 from being able to bind the antibody-antigen complex.

Given the range of serious diseases mediated by a disfunction of the complement system, there is a clear medical need to provide pharmaceutically acceptable compounds, methods, compositions, and methods of manufacture to inhibit the complement system in a patient in need thereof.

Therefore, the present disclosure provides compounds and their uses and compositions to treat disorders arising from or amplified by a disfunction of the complement system. The present disclosure also provides compounds, uses, compositions, combinations, and processes of manufacture that inhibit C1s (complement C1 esterase) and thus can treat disorders mediated by C1s.

### SUMMARY

The present disclosure provides compounds, compositions, and methods for treating a disorder mediated by the complement cascade (including a dysfunctional cascade), a disorder or abnormality of a cell that adversely affects the ability of the cell to engage in or respond to normal complement activity including for example, the classical complement pathway, or an undesired complement-mediated response to a medical treatment, such as surgery or other medical procedure or a pharmaceutical or biopharmaceutical drug administration, a blood transfusion, or other allogenic tissue or fluid administration. In some embodiments, the active compound may act as an inhibitor of the complement classical pathway by inhibiting complement C1s.

Without wishing to be bound by theory, the present disclosure is based, in part, on the unexpected discovery that compounds of the disclosure exhibit advantageous properties over structurally related compounds (e.g., the compounds described in WO 2020/198062 and WO2022/066774), such as improved CIs inhibiting activity, improved classical pathway hemolysis inhibiting activity, improved Caco-2 permeability, improved oral bioavailability, improved C1s selectivity (e.g., over other proteases, such as MASP-2) and/or improved metabolic stability.

In one aspect, the present disclosure provides a compound of formula (I):

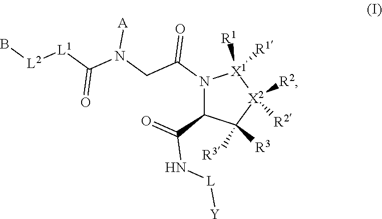

or a pharmaceutically acceptable salt thereof, in which variables A, B, L¹, L², X¹, X², R¹, R^{1′}, R², R^{2′}, R³, R^{3′}, L, and Y are as defined herein.

In another aspect, the present disclosure provides a compound of Table 1, or a pharmaceutically acceptable salt thereof.

In another aspect, the present disclosure provides a pharmaceutical composition including a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof, and a pharmaceutically acceptable excipient.

In another aspect, the present disclosure provides a complement C1 esterase (C1s) mediated disorder. The method includes administering to a subject, e.g., a human subject, in need thereof a therapeutically effective amount of a compound disclosed herein (e.g., any one of the compounds of formulas (I), (II), (III), (IIA), (IV), (IVA), (V), (VA), (VI), (VIA), (VII), and (VIIA), and Table 1) or a pharmaceutically acceptable salt thereof.

In another aspect, the present disclosure provides a compound disclosed herein (e.g., any one of the compounds of formulas (I), (IL), (III), (IIIA), (IV), (IVA), (V), (VA), (VI), (VIA), (VII), and (VIIA), and Table 1) or a pharmaceutically acceptable salt thereof, for use in the treatment of a CIs mediated disorder.

In another aspect, the present disclosure provides a use of a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof in the preparation a medicament for use in the treatment of a C1s mediated disorder.

### Definitions

Compounds are described using standard nomenclature. Unless defined otherwise, all technical and scientific terms used herein have the same meaning as is commonly understood by one of skill in the art to which the present disclosure belongs.

The terms “a” and “an” do not denote a limitation of quantity, but rather denote the presence of at least one of the referenced items. The term “or” means “and/or.” Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely as illustration, and does not pose a limitation on the scope of the invention.

The term “alkoxy,” as used herein, refers to a —OR radical, in which R is alkyl, as defined herein.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-8, 1-6, 1-4, or 1-2 carbon atoms (e.g., C₁-C₈, C₁-C₆, C₁-C₄, or C₁-C₂). Examples include, but are not limited to, methyl, ethyl, isobutyl, sec-butyl, tert-butyl, 2-methylpropyl, and 2,2-dimethylpropyl.

The term “alkylene,” as used herein, refers to a divalent radical obtained by removing a hydrogen atom from a carbon atom of an alkyl group. The divalency of an alkylene group does not include the optional substituents on the alkylene group. Examples of alkylene groups include, but are not limited to, methylene, ethylene, and n-propylene.

The term “amino,” as used herein, refers to a monovalent radical of formula —NH₂. An “optionally substituted amino,” as used herein, refers to an amino group in which one or both hydrogen atoms are independently replaced with a substituent as defined herein.

The term “aryl,” as used herein, refers to any monocyclic or fused ring bicyclic or multicyclic system containing only carbon atoms in the ring(s), which has the characteristics of aromaticity in terms of electron distribution throughout the entire ring system, e.g., phenyl, naphthyl, or phenanthryl. An aryl group may have, e.g., 6-16, 6-14, or 6-10 carbon ring atoms (e.g., C₆-C₁₆, C₆-C₁₄, C₆-C₁₀, C₆, C₁₀, C₁₄, or C₁₆).

The term “arylene,” as used herein refers to a divalent radical obtained by removing a hydrogen atom from a carbon atom of an aryl group. The divalency of an arylene group does not include the optional substituents on the arylene group. Phenylene is a non-limiting example of an arylene group.

The term “aryloxy,” as used herein, refers to an —OR radical, in which R is aryl, as defined herein.

The term “carbocyclyl,” as used herein, refers to a monovalent, saturated (i.e., cycloalkyl) or unsaturated, non-aromatic group (e.g., cycloalkenyl, which contains at least one carbon-carbon double bond and no carbon-carbon triple bonds) containing only C and H when unsubstituted, which may be monocyclic, bicyclic, or multicyclic (e.g., tricyclic). A carbocyclyl may have, e.g., 3-14 carbons (e.g., a C₃-C₄, C₃-C₅, C₃-C₆, C₃-C₇, C₃-C₈, or C₃-C₁₄ carbocyclyl). Examples of carbocyclyls include, but are not limited to, cyclopropyl, cyclobutyl, cyclopentyl, cyclohexyl, cycloheptyl, cyclohexenyl, cycloheptenyl, and fluorenyl. The term “carbocyclyl” also includes cyclic groups having a bridged multicyclic structure in which one or more carbons bridges two non-adjacent members of a monocyclic ring, e.g., bicyclo[2.2.1]heptyl.

The term “cycloalkyloxy,” as used herein, refers to an —OR radical, in which R is cycloalkyl, as defined herein.

The term “halo,” as used herein, refers to a fluorine (fluoro; F), chlorine (chloro; Cl), bromine (bromo; Br), or iodine (iodo; I) radical.

The term “heteroaryl,” as used herein, refers to a monocyclic, bicyclic, or multicyclic aromatic ring monocyclic, bicyclic, or multicyclic group containing 1, 2, 3, or 4 heteroatoms selected from N, O, S, B, and P (e.g., 1-4, 1-3, or 1 or 2 heteroatoms selected from N, O, and S) as ring atoms, with the remaining ring atoms being carbon. In some embodiments, a heteroaryl group is a bicyclic or tricyclic system containing at least one 5, 6, or 7 membered aromatic ring which contains from 1, 2, 3, or 4 heteroatoms selected from N, O, S, B or P (e.g., 1-4, 1-3, or 1 or 2 heteroatoms selected from N, O, and S) as ring atoms, with the remaining ring atoms being carbon. In some embodiments, a heteroaryl group is a monocyclic aromatic ring having 5 or 6 ring atoms (i.e., 5-or 6-membered heteroaryl). In some embodiments, is a bicyclic aromatic ring system having 8 to 10 ring atoms (i.e., 8-to 10-membered bicyclic heteroaryl). Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, napthyridinyl, tetrahydrofuranyl, and furopyridinyl.

The term “heteroaryloxy,” as used herein, refers to a monovalent radical of formula —OR, in which R is heteroaryl, as defined herein.

The term “heterocyclyl,” as used herein, refers to saturated or unsaturated, non-aromatic, monocyclic, bicyclic, or multicyclic group containing 1, 2, 3, or 4 heteroatoms selected from N, O, S, B, and P (e.g., 1-4, 1-3, or 1 or 2 heteroatoms selected from N, O, and S) as ring atoms, with the remaining ring atoms being carbon. The term “heterocyclyl” includes, e.g., monocyclic 3-to 12-membered rings, bicyclic 5-to 16-membered ring systems, multicyclic (e.g., tricyclic) 10-to 18-membered ring systems, which may include bridged ring systems when bicyclic or multicyclic. In some embodiments, a heterocyclyl group contains 3-16 ring atoms (i.e., 3-to 16-membered heterocyclyl), e.g., 3-12 ring atoms (i.e., 3-to 12-membered heterocyclyl) or 4-10 ring atoms (i.e., 4-to 10-membered heterocyclyl). Examples of saturated heterocyclyl groups include saturated 4-to 7-membered monocyclic groups containing 1 to 4 nitrogen atoms (e.g., pyrrolidinyl, imidazolidinyl, piperidinyl, pyrrolinyl, azetidinyl, piperazinyl, and pyrazolidinyl); saturated 4 to 6-membered monocyclic groups containing 1 to 2 oxygen atoms and 1 to 3 nitrogen atoms (e.g., morpholinyl); saturated 3 to 6-membered monocyclic groups containing 1 to 2 sulfur atoms and 1 to 3 nitrogen atoms (e.g., thiazolidinyl). Examples of unsaturated, non-aromatic heterocyclyl radicals include but are not limited to, dihydrothienyl, dihydropyranyl, dihydrofuryl, and dihydrothiazolyl. Other examples of heterocyclyl radicals include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ-benzo[d]isothiazol-6-yl, dihydropyranyl, dihydrofuryl and dihydrothiazolyl. “Bicyclic heterocyclyl” includes groups in which a saturated or unsaturated, non-aromatic ring containing 1, 2, 3, or 4 heteroatoms as ring atoms is fused with an aryl group (e.g., phenyl) or a cycloalkyl group. “Bicyclic heterocyclyl” also includes groups in which a heteroaryl group, as defined herein, is fused to a saturated or unsaturated, non-aromatic ring containing 0, 1, 2, 3, or 4 heteroatoms as ring atoms.

The term “heterocyclyloxy,” as used herein, refers to a monovalent radical of formula —OR, in which R is heterocyclyl, as defined herein.

The term “oxo,” as used herein, refers to a ═O radial.

The term “substituted”, as used herein, means that any one or more hydrogens on the designated atom or group is replaced with a moiety as defined herein or selected from an indicated group of moieties, provided that the designated atom's normal valence is not exceeded, and the resulting compound is stable. For example, when the substituent is oxo (i.e., ═O), then two hydrogens on the atom are replaced. For example, a pyridyl group substituted by oxo is a pyridone. Combinations of substituents and/or variables are permissible only if such combinations result in stable compounds or useful synthetic intermediates. The phrase “optionally substituted X,” as used herein, is intended to be equivalent to “X, in which X is optionally substituted” (e.g., “alkyl, in which said alkyl is optionally substituted”). It is not intended to mean that the feature “X” (e.g., alkyl) per se is optional. The term “optionally substituted,” as used herein, refers to having 0, 1, or more substituents (e.g., 0-10 substituents, 0-5 substituents, or 0, 1, 2, 3, 4, 5, 6, 7, 8, 9, or 10 substituents).

Alkyl, alkylene, alkoxy, amino, carbocyclyl, aryl, arylene, aryloxy, heteroaryl, and heterocyclyl groups may be substituted with carbocyclyl (e.g., cycloalkyl); aryl; heteroaryl; heterocyclyl; halo; OR, in which R is H, alkyl, carbocyclyl (e.g., cycloalkyl), aryl, heteroaryl, or heterocyclyl; SR, in which R is H, alkyl, cycloalkyl, aryl, heteroaryl, or heterocyclyl; CN; NO₂; N₃; NRR′; in which each of R and R′ is, independently, H, alkyl, cycloalkyl, aryl, heteroaryl, or heterocyclyl; SO₂R, in which R is H, alkyl, or aryl; SO₂NRR′, in which each of R and R′ is, independently, II, alkyl, or aryl; SOR, in which R is II, alkyl, or aryl; or P(O)(OR)₂, in which each R is, independently, H or alkyl. Amino, aryl, carbocyclyl, heteroaryl, and heterocyclyl groups may also be substituted with alkyl. Alkyl, alkylene, carbocyclyl, and heterocyclyl groups may also be substituted with oxo or ═NR, in which R is H or alkyl. Alkyl and alkylene groups may also be substituted with spirocyclic carbocycle (e.g., spirocyclic cycloalkyl) or spirocyclic heterocyclyl. In some embodiments, a substituent is further substituted with one or more substituents as described herein. For example, a C₁ alkyl group, i.e., methyl, may be substituted with oxo to form a formyl group and further substituted with —OH or —NR₂ to form a carboxyl group or an amido group.

The term “complement-mediated disorder,” as used herein, refers to a disorder in which the amount or activity of complement is such as to cause disorder in an individual.

As used herein, a compound having “complement Cl esterase (C1s) inhibiting activity” refers to a compound exhibiting an IC₅₀ of less than 100 nM against as determined with a human complement C1s enzyme assay as described in Example 3 herein.

The term “pharmaceutical composition,” as used herein, refers to one or more active compounds, formulated together with one or more pharmaceutically acceptable excipients. In some embodiments, a compound of the disclosure (e.g., is present in a unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, or capsules; and parenteral administration, for example, by subcutaneous, intramuscular, or intravenous injection.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid.

The term “pharmaceutically acceptable excipient,” as used herein, refers to any inactive ingredient (for example, a vehicle capable of suspending or dissolving the active compound) that is biocompatible and suitable for administration to a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Those of ordinary skill in the art are familiar with a variety of agents and materials useful as excipients.

The term “subject,” as used herein, can be a human, non-human primate, or other non-human mammal, such as but not limited to dog, cat, horse, cow, pig, goat, monkey, rat, mouse, and sheep. In preferred embodiments, the subject is a human.

As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms or conditions; diminishment of extent of disease, disorder, or condition; stabilizing (i.e., not worsening) of the state of disease, disorder, or condition; delay or slowing in the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or the time course of the progression is slowed or lengthened, as compared to the extent or time course in the absence of treatment.

A “therapeutically effective amount” or an “effective amount” of a compound or pharmaceutical composition of the present disclosure refers an amount effective, when administered to a subject, to provide a therapeutic benefit, such as an amelioration of symptoms or reduction or diminution of the disease itself. In one embodiment, a therapeutically effective amount is an amount sufficient to prevent a significant increase, or will significantly reduce, the detectable level of hemolysis in the patient's blood, serum, or tissues.

### DETAILED DESCRIPTION

### Active Compounds

The present disclosure provides compounds and salts useful for the treatment of a disorder mediated by the complement cascade (e.g., a disorder mediated by C1s). In one aspect, the present disclosure provides a compound of Formula (I):

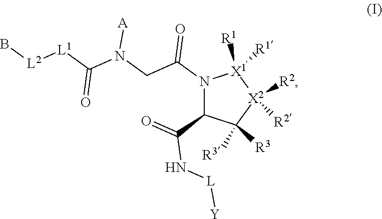

or a pharmaceutically acceptable salt thereof, in which:
X¹ is C or N;
X² is C, S, or Si;
each of R¹, R^{1′}, R², R^{2′}, R³, and R^{3′} is independently selected from H, halo, hydroxy, optionally substituted C₁-C₆ alkyl, optionally substituted C₃-C₈ cycloalkyl, optionally substituted C₆-C₁₄ aryl, optionally substituted 5-to 10-membered heterocycle, or optionally substituted 5-to 10-membered heteroaryl; optionally substituted C₁-C₆ alkoxy; optionally substituted C₆-C₁₄ aryloxy, (optionally substituted 5-to 10-membered heteroaryl)oxy; SO₂R^{a}, in which R^{a} is H, C₁-C₆ alkyl, or C₃-C₈ cycloalkyl; S(O)(NH)R^{b}, in which R^{b} is H or C₁-C₆ alkyl, where, when X¹ is N, R¹ or R^{1′} is absent and when X² is S, R² and R^{2′} are absent; or
R¹ and R², together with the atoms to which each is attached, form optionally substituted C₃-C₈ cycloalkyl; or
R¹ and R² combine to form a double bond; R′″ and R^{2′}, together with the atoms to which each is attached, form optionally substituted C₃-C₈ cycloalkyl; or
R² and R^{2′}, together with the atom to which they are attached, form optionally substituted 4-or 5-membered spirocyclic heterocycle; or
R² and R^{2′}, together with the atom to which they are attached, form optionally substituted C₃-C₈ spirocyclic cycloalkyl; or
R² and R^{2′} combine to form ═C(R^{c})₂, in which each R^{c} is independently H or halo;
Y is

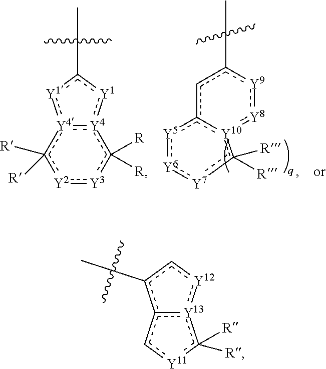

in which:
   Y¹ is O, S, NR^{d}, in which each R^{d} is independently absent, H, or C₁-C₆ alkyl;
   Y^{1′} is O, S, NR^{d}, or C(R^{d})₂;
   each of Y² and Y³ is independently NR^{e} or C(R^{e})₂, in which each R^{e} is independently absent; H; optionally substituted C₁-C₆ alkyl; halo; or N(R^{g})₂, in which each Rⁱ is independently H or C₁-C₆ alkyl; or both R^{e} combine to form oxo;
   each of Y⁴, Y^{4′}, Y¹⁰, and Y¹³ is independently CR^{e} or N;
   each of Y⁵, Y⁶, and Y⁷ is independently O, S, NR^{f} or C(R^{f})₂, in which each R^{f} is independently absent; H; optionally substituted C₁-C₆ alkyl; halo; or N(R^{g})₂; or both R^{f} combine to form oxo;
   each of Y⁸ and Y⁹ is independently C(R^{f})₂ or NR^{f}; each of Y¹¹ and Y¹² is independently NR^{e}, C(R^{e})₂, S, or O; each [symbol] is independently a single bond or a double bond;
   each of R, R′, R″, and R′″ is independently absent, II, optionally substituted C₁-C₆ alkyl, halo, or N(R^{g})₂; or
   both R combine to form oxo; or
   both R¹ combine to form oxo; and
   q is 0 or 1;
L is selected from a bond,

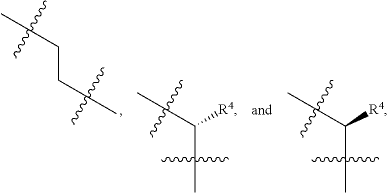

in which R⁴ is H, CH₃, CF₃, or CH₂OH;
L¹ is a bond or optionally substituted C₁-C₆ alkylene;
L² is a bond or O;
B is optionally substituted C₆-C₁₄ aryl; optionally substituted C₃-C₁₄ carbocyclyl; optionally substituted 5-to 14-membered heterocyclyl; or optionally substituted 5-to 10-membered heteroaryl, and
A is H or C₁-C₆ alkyl; or
A is C₁-C₂ alkylene bound to a ring atom in B.

In some embodiments, Y is

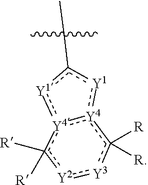

In some embodiments, Y is

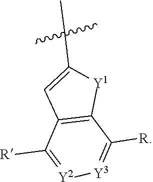

In some embodiments, Y² is N. In some embodiments, Y² is CR^{e}. In some embodiments, Y² is CH. In some embodiments, Y² is CNH₂. In some embodiments, Y³ is N. In some embodiments, Y³ is CR^{e}. In some embodiments, Y³ is CH. In some embodimens, Y³ is CNH₂.

In some embodiments, Y is

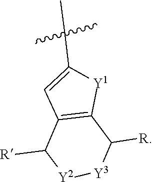

In some embodiments, Y² is NR^{e}. In some embodiments, Y² is NH. In some embodiments, Y² is NCH₃. In some embodiments, Y² is C(R^{c})₂. In some embodiments, Y² is CH₂. In some embodiments, Y² is CHCH₃. In some embodiments, Y² is C(O). In some embodiments, Y³ is NR^{e}. In some embodiments, Y³ is NH. In some embodiments, Y³ is NCH₃. In some embodiments, Y³ is C(R)₂. In some embodiments, Y³ is CH₂. In some embodiments, Y³ is CHCH₃. In some embodiments, Y³ is C(O).

In some embodiments, in which Y is

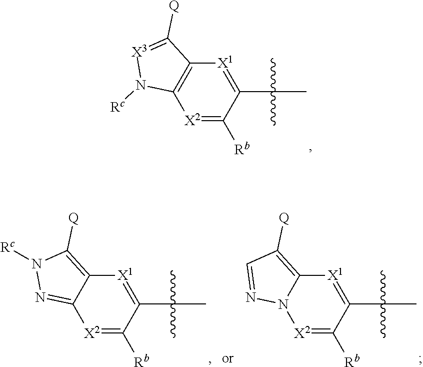

Y¹ is NR^{d}. In some embodiments, Y¹ is NH. In some embodiments, Y¹ is NCH₃. In some embodiments, Y¹ is O. In some embodiments, Y¹ is S.

In some embodiments, Y is

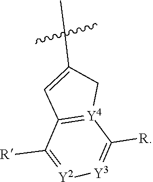

In some embodiments, Y⁴ is N. In some embodiments, Y² is N. In some embodiments, Y² is CR^{e}. In some embodiments, Y2 is CH In some embodiments, Y³ is N. In some embodiments, Y³ is CR^{e}. In some embodiments, Y³ is CH.

In some embodiments, R is H. In some embodiments, R is CH₃. In some embodiments, R is F. In some embodiments. R is Cl.

In some embodiments, R′ is H. In some embodiments, R′ is CH₃. In some embodiments, R′ is NH₂.

In some embodiments, Y is

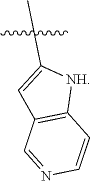 [SMILES: *c1cc2cnccc2[nH]1]

In some embodiments, Y is

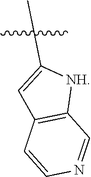 [SMILES: *c1cc2ccncc2[nH]1]

In some embodiments, Y is

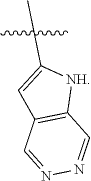 [SMILES: *c1cc2cnncc2[nH]1]

In some embodiments, Y is

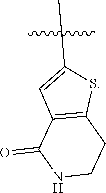 [SMILES: *c1cc2c(s1)CCNC2=O]

In some embodiments, Y is

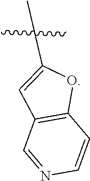 [SMILES: *c1cc2cnccc2o1]

In some embodiments, Y is

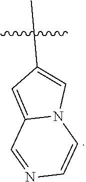 [SMILES: *c1cc2cnccn2c1]

In some embodiments, Y is

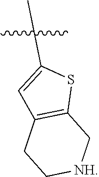 [SMILES: *c1cc2c(s1)CNCC2]

In some embodiments, Y is

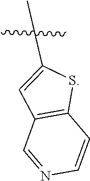 [SMILES: *c1cc2cnccc2s1]

In some embodiments, Y is

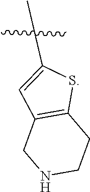 [SMILES: *c1cc2c(s1)CCNC2]

In some embodiments, Y is

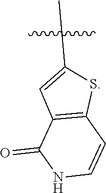 [SMILES: *c1cc2c(=O)[nH]ccc2s1]

In some embodiments, Y is

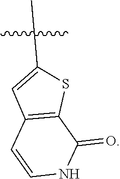 [SMILES: *c1cc2cc[nH]c(=O)c2s1]

In some embodiments, Y is

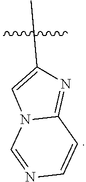 [SMILES: *c1cn2cnccc2n1]

In some embodiments, Y is

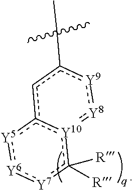

In some embodiments, Y is

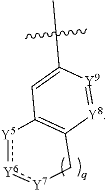

In some embodiments, q is 0. In some embodiments, q is 1. In some embodiments, Y⁹ is C(R^{f})₂. In some embodiments, Y⁹ is CH. In some embodiments, Y⁸ is C(R^{f})₂. In some embodiments, Y⁸ is CH. In some embodiments, Y⁸ is NR^{f}. In some embodiments, Y⁸ is N. In some embodiments, Y⁵ is NR^{f}. In some embodiments, Y⁵ is NH. In some embodiments, Y⁶ is C(R^{f})₂. In some embodiments, Y⁶ is CH. In some embodiments, Y⁶ is C(NH₂). In some embodiments, Y⁶ is NR^{f}. In some embodiments, Y⁶ is NH. In some embodiments, Y⁷ is NR^{f}. In some embodiments, Y⁷ is N. In some embodiments, Y⁷ is NH. In some embodiments, Y⁷ is C(R^{f})₂. In some embodiments, Y⁷ is CH. In some embodiments, Y⁶ is CH₂.

In some embodiments, Y is

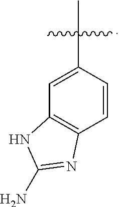 [SMILES: *c1ccc2nc(N)[nH]c2c1]

In some embodiments, Y is

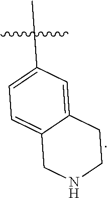 [SMILES: *c1ccc2c(c1)CCNC2]

In some embodiments, Y is

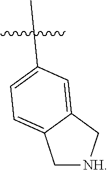 [SMILES: *c1ccc2c(c1)CNC2]

In some embodiments, Y is

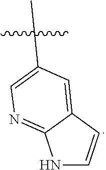 [SMILES: *c1cnc2[nH]ccc2c1]

In some embodiments, Y is

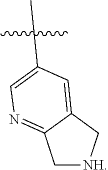 [SMILES: *c1cnc2c(c1)CNC2]

In some embodiments, Y is

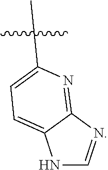 [SMILES: *c1ccc2[nH]cnc2n1]

In some embodiments, Y is

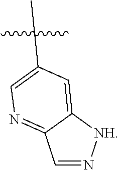 [SMILES: *c1cnc2cn[nH]c2c1]

In some embodiments, Y is

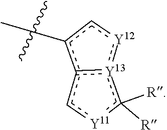

In some embodiments, Y is

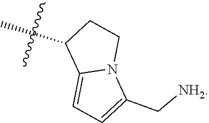

In some embodiments, the compound is a compound of Formula (II):

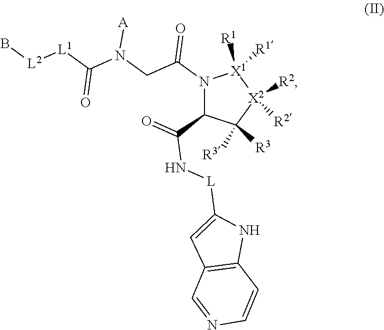

or a pharmaceutically acceptable salt thereof, in which all variables are as defined for Formula (I).

In some embodiments, B is optionally substituted C₆-C₁₄ aryl or optionally substituted 5-to 10-membered heteroaryl.

In some embodiments, the compound is a compound of formula (III):

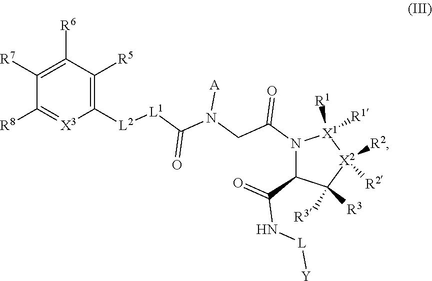

or a pharmaceutically acceptable salt thereof, in which
X³ is CR⁹ or N;
each of R⁵, R⁶, and R⁹ is independently selected from H, halo, CN, SF₅, optionally substituted C₁-C₆ alkyl, optionally substituted C₁-C₆ alkoxy, S(O)(NH)CH₃, S(O)₂CH₃, and S(O)(NCN)CH₃;
each of R⁷ and R⁸ is independently H, halo, CN, SF₅, optionally substituted C₁-C₆ alkyl, optionally substituted C₁-C₆ alkoxy, optionally substituted amino, S(O)(NH)CH₃, S(O)₂CH₃, S(O)(NCN)CH₃, optionally substituted C₃-C₈ cycloalkyl, optionally substituted C₃-C₈ cycloalkyloxy, optionally substituted C₆-C₁₄ aryloxy, optionally substituted C₆-C₁₄ aryl, optionally substituted 5-to 10-membered heterocyclyl, optionally substituted 5-to 10-membered heteroaryl, optionally substituted (5-to 10-membered heteroaryl)oxy, or optionally substituted (4-to 10-membered heterocyclyl)oxy, provided that no more than one of R⁷ and R⁸ is optionally substituted C₆-C₁₄ aryloxy, optionally substituted C₆-C₁₄ aryl, optionally substituted 5-to 10-membered heteroaryl, optionally substituted (5-to 10-membered heteroaryl)oxy, or optionally substituted (4-to 10-membered heterocyclyl)oxy; or
R⁷ and R⁸, together with the atoms to which each is attached, form optionally substituted 5-to 6-membered heterocyclyl, optionally substituted 5-to 10-membered heteroaryl, or optionally substituted C₆-C₁₄ aryl; or
R⁵ and A combine to form optionally substituted C₁-C₂ alkylene; or
R⁶ and R⁹ combine to form (C₂-C₆alkylene)(C₆-C₁₄ arylene)(C₂-C₆alkylene), and each of R⁵, R⁷, and R⁸ is H; and all other variables are as defined for Formula (I).

In some embodiments, the compound is a compound of Formula (IIIA):

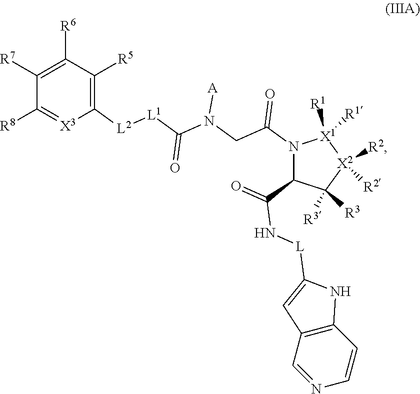

or a pharmaceutically acceptable salt thereof, in which all variables are as defined for Formula (l).

In some embodiments, X³ is CR⁹. In some embodiments, X³ is CH. In some embodiments, X³ is CF. In some embodiments, X³ is N. In some embodiments, R⁷ is optionally substituted C₆-C₁₄ aryl. In some embodiments, R⁷ is optionally substituted phenyl. In some embodiments, R⁷ is

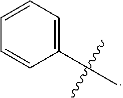 [SMILES: *c1ccccc1]

In some embodiments, R⁷ is

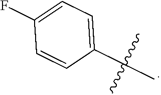 [SMILES: *c1ccc(F)cc1]

In some embodiments, R⁷ is

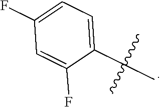 [SMILES: *c1ccc(F)cc1F]

In some embodiments, R⁷ is

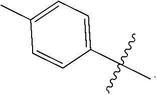 [SMILES: *c1ccc(C)cc1]

In some embodiments, R⁷ is

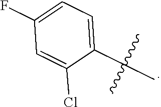 [SMILES: *c1ccc(F)cc1Cl]

In some embodiments, R⁷ is optionally substituted 5-to 10-membered heteroaryl. In some embodiments, R⁷ is

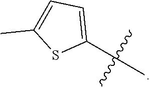 [SMILES: *c1ccc(C)s1]

In some embodiments, R⁷ is optionally substituted C₃-C₈ cycloalkyloxy. In some embodiments, R⁷ is

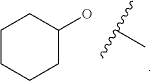

In some embodiments, R⁸ is halo. In some embodiments, R⁸ is Br. In some embodiments, R⁸ is C₁. In some embodiments, R⁸ is F. In some embodiments, R⁸ is H. In some embodiments, R⁸ is optionally substituted C₁-C₆ alkyl. In some embodiments, R⁸ is optionally substituted CH₃.

In some embodiments, R⁸ is halo. In some embodiments, R⁸ is Br. In some embodiments, R⁸ is C₁. In some embodiments, R⁸ is F. In some embodiments, R⁸ is H. In some embodiments, R⁸ is optionally substituted C₁-C₆ alkyl. In some embodiments, R⁸ is optionally substituted CH₃.

In some embodiments, R⁸ is optionally substituted C₆-C₁₄ aryl. In some embodiments, R⁸ is optionally substituted phenyl. In some embodiments. R⁸ is

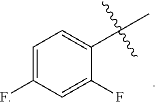 [SMILES: *c1ccc(F)cc1F]

In some embodiments, R⁸ is optionally substituted C₆-C₁₄ aryloxy. In some embodiments, R⁸ is optionally substituted phenoxy. In some embodiments, R⁸ is

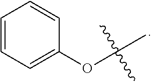 [SMILES: *Oc1ccccc1]

In some embodiments, R⁸ is

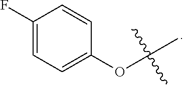 [SMILES: *Oc1ccc(F)cc1]

In some embodiments, R⁸ is

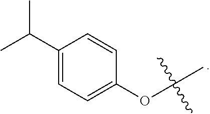 [SMILES: *Oc1ccc(C(C)C)cc1]

In some embodiments, R⁸ is

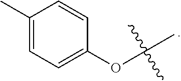 [SMILES: *Oc1ccc(C)cc1]

In some embodiments, R⁸ is

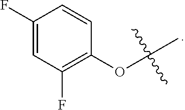 [SMILES: *Oc1ccc(F)cc1F]

In some embodiments, R⁸ is

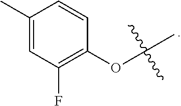 [SMILES: *Oc1ccc(C)cc1F]

In some embodiments, R⁸ is

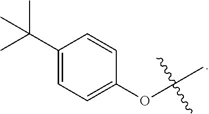 [SMILES: *Oc1ccc(C(C)(C)C)cc1]

In some embodiments, R⁸ is

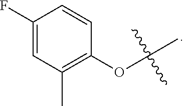 [SMILES: *Oc1ccc(F)cc1C]

In some embodiments, R⁸ is

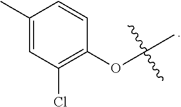 [SMILES: *Oc1ccc(C)cc1Cl]

In some embodiments, R⁸ is

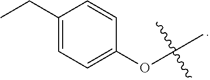 [SMILES: *Oc1ccc(CC)cc1]

In some embodiments, R⁸ is

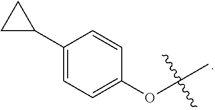 [SMILES: *Oc1ccc(C2CC2)cc1]

In some embodiments, R⁸ is

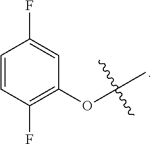 [SMILES: *Oc1cc(F)ccc1F]

In some embodiments, R⁸ is

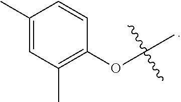 [SMILES: *Oc1ccc(C)cc1C]

In some embodiments, R⁸ is

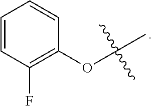 [SMILES: *Oc1ccccc1F]

In some embodiments, R⁸ (optionally substituted 5-to 10-membered heteroaryl)oxy. In some embodiments, R⁸ is

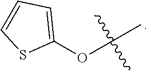 [SMILES: *Oc1cccs1]

In some embodiments, R⁸ is

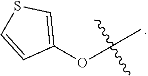 [SMILES: *Oc1ccsc1]

In some embodiments, R⁸ is

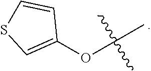 [SMILES: *Oc1ccsc1]

In some embodiments, R⁸ is

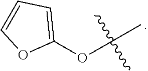 [SMILES: *Oc1ccco1]

In some embodiments, R⁸ is

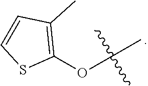 [SMILES: *Oc1sccc1C]

In some embodiments, R⁸ is optionally substituted 5-to 10-membered heteroaryl. In some embodiments, R⁸ is

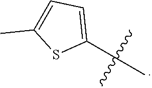 [SMILES: *c1ccc(C)s1]

In some embodiments, R⁸ is optionally substituted C₁-C₆ alkoxy. In some embodiments, R⁸ is

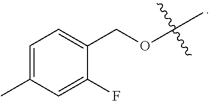 [SMILES: *OCc1ccc(C)cc1F]

In some embodiments, R⁸ is optionally substituted 5-to 10-membered heterocyclyl. In some embodiments, R⁸ is

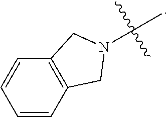 [SMILES: *N1Cc2ccccc2C1]

In some embodiments, R⁷ and R⁸, together with the atoms to which each is attached, form optionally substituted 5-to 6-membered heterocyclyl. In some embodiments, R⁷ and R⁸, together with the atoms to which each is attached, form

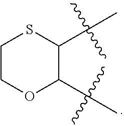 [SMILES: *C1OCCSC1*]

In some embodiments, R⁶ is H. In some embodiments, R⁶ is halo. In some embodiments, R⁶ is F. In some embodiments, R⁶ is optionally substituted C₁-C₆ alkyl. In some embodiments, R⁶ is CH₃.

In some embodiments, R⁵ is H. In some embodiments, R⁵ is halo. In some embodiments, R⁵ is F. In some embodiments, R⁵ is optionally substituted C₁-C₆ alkoxy. In some embodiments, R⁵ is OCH₃. In some embodiments, R⁵ and A combine to form optionally substituted C₁-C₂ alkylene. In some embodiments, R⁵ and A combine to form —CH₂—.

In some embodiments, the compound is a compound of Formula (IV):

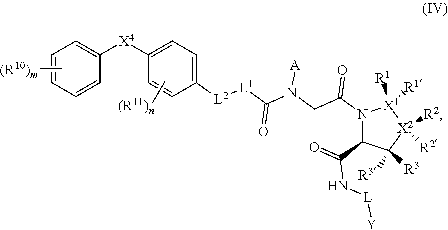

or a pharmaceutically acceptable salt thereof, in which:
X⁴ is O; C(R^{h})₂, in which each R^{h} is independently hydrogen, halo, or optionally substituted C₁-C₆ alkyl, or both R^{h} combine to form oxo; S(O)₂, or NR^{h};
m is selected from 0, 1, 2, 3, 4, and 5;
n is selected from 0, 1, 2, 3, and 4;
each R¹⁰ and R¹¹ is independently halo, CN, optionally substituted C₁-C₆ alkyl, optionally substituted C₁-C₆ alkoxy, or optionally substituted C₃-C₈ cycloalkyl, and all other variables are as defined for Formula (I).

In some embodiments, the compound is a compound of Formula (IVA):

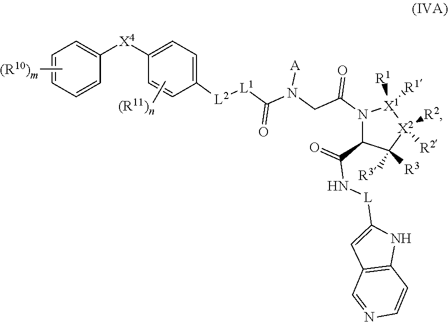

or a pharmaceutically acceptable salt thereof, in which all variables are as defined for Formula (IV).

In some embodiments, in which the compound is a compound of Fomrula (IV) or (IVA) or a pharmaceutically acceptable salt thereof, X⁴ is O. In some embodiments, B is

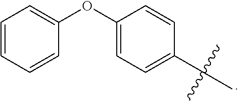 [SMILES: *c1ccc(Oc2ccccc2)cc1]

In some embodiments, B is

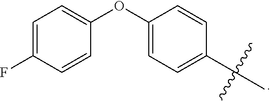 [SMILES: *c1ccc(Oc2ccc(F)cc2)cc1]

In some embodiments, B is

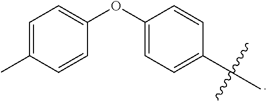 [SMILES: *c1ccc(Oc2ccc(C)cc2)cc1]

In some embodiments, B is

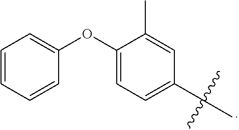 [SMILES: *c1ccc(Oc2ccccc2)c(C)c1]

In some embodiments, B is

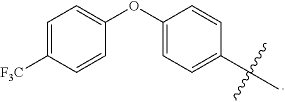 [SMILES: *c1ccc(Oc2ccc(C(F)(F)F)cc2)cc1]

In some embodiments, B is

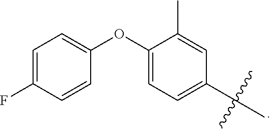 [SMILES: *c1ccc(Oc2ccc(F)cc2)c(C)c1]

In some embodiments, B is

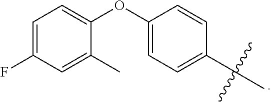 [SMILES: *c1ccc(Oc2ccc(F)cc2C)cc1]

In some embodiments, B is

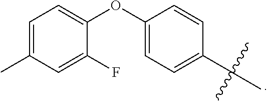 [SMILES: *c1ccc(Oc2ccc(C)cc2F)cc1]

In some embodiments, in which the compound is a compound of Fomrula (IV) or (IVA) or a pharmaceutically acceptable salt thereof, X⁴ is C(R^{h})₂. In some embodiments, in which the compound is a compound of Fomrula (IV) or (IVA), X⁴ is C(O). In some embodiments, B is

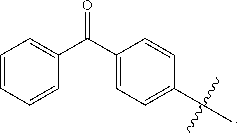 [SMILES: *c1ccc(C(=O)c2ccccc2)cc1]

In some embodiments, the compound is a compound of Formula (V):

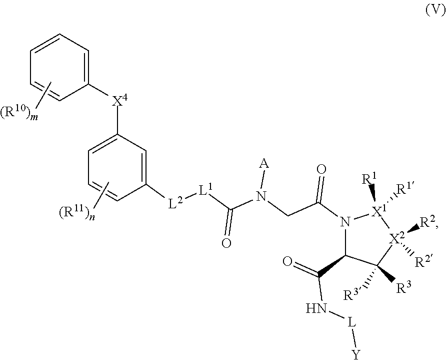

or a pharmaceutically acceptable salt thereof, in which:
X⁴ is O; C(R^{h})₂, in which each R^{h} is independently hydrogen, halo, or optionally substituted C₁-C₆ alkyl, or both R^{h} combine to form oxo; S(O)₂, or NR^{h};
m is selected from 0, 1, 2, 3, 4, and 5;
n is selected from 0, 1, 2, 3, and 4; and
each R¹⁰ and R¹¹ is independently halo, CN, optionally substituted C₁-C₆ alkyl, optionally substituted C₁-C₆ alkoxy, or optionally substituted C₃-C₈ cycloalkyl, and all other variables are as defined for Formula (I).

In some embodiments, the compound is a compound of Formula (VA):

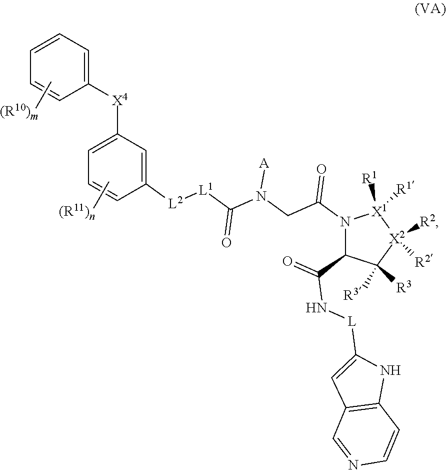

or a pharmaceutically acceptable thereof, in which all variables are as defined for Formula (V).

In some embodiments, in which the compound is a compound of Formula (V) or (VA) or a pharmaceutically acceptable salt thereof, X⁴ is O. In some embodiments, B is

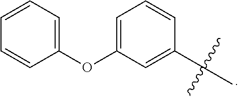 [SMILES: *c1cccc(Oc2ccccc2)c1]

In some embodiments, B is

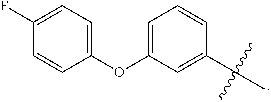 [SMILES: *c1cccc(Oc2ccc(F)cc2)c1]

In some embodiments, B is

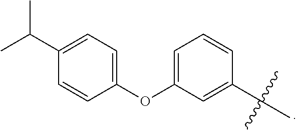 [SMILES: *c1cccc(Oc2ccc(C(C)C)cc2)c1]

In some embodiments, B is

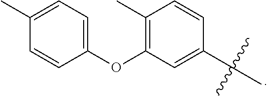 [SMILES: *c1ccc(C)c(Oc2ccc(C)cc2)c1]

In some embodiments, B is

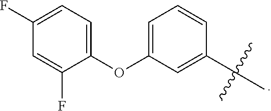 [SMILES: *c1cccc(Oc2ccc(F)cc2F)c1]

In some embodiments, B is

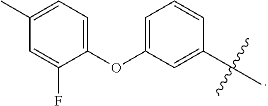 [SMILES: *c1cccc(Oc2ccc(C)cc2F)c1]

In some embodiments, B is

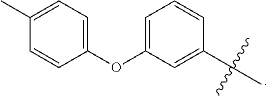 [SMILES: *c1cccc(Oc2ccc(C)cc2)c1]

In some embodiments, B is

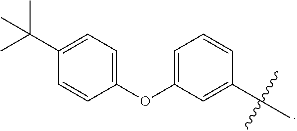 [SMILES: *c1cccc(Oc2ccc(C(C)(C)C)cc2)c1]

In some embodiments, B is

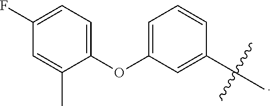 [SMILES: *c1cccc(Oc2ccc(F)cc2C)c1]

In some embodiments, B is

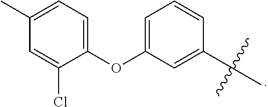 [SMILES: *c1cccc(Oc2ccc(C)cc2Cl)c1]

In some embodiments, B is

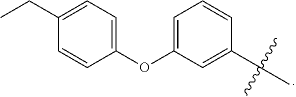 [SMILES: *c1cccc(Oc2ccc(CC)cc2)c1]

In some embodiments, B is

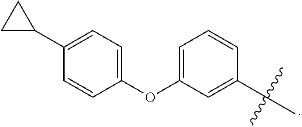 [SMILES: *c1cccc(Oc2ccc(C3CC3)cc2)c1]

In some embodiments, B is

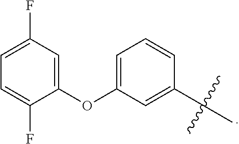 [SMILES: *c1cccc(Oc2cc(F)ccc2F)c1]

In some embodiments, B is

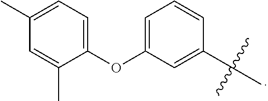 [SMILES: *c1cccc(Oc2ccc(C)cc2C)c1]

In some embodiments, B is

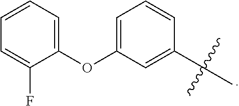 [SMILES: *c1cccc(Oc2ccccc2F)c1]

In some embodiments, B is

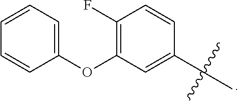 [SMILES: *c1ccc(F)c(Oc2ccccc2)c1]

In some embodiments, B is

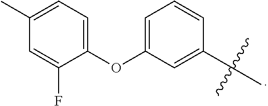 [SMILES: *c1cccc(Oc2ccc(C)cc2F)c1]

In some embodiments, B is

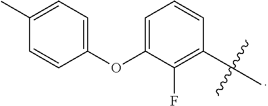 [SMILES: *c1cccc(Oc2ccc(C)cc2)c1F]

In some embodiments, B is

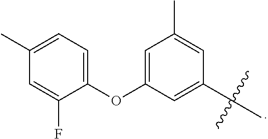 [SMILES: *c1cc(C)cc(Oc2ccc(C)cc2F)c1]

In some embodiments, B is

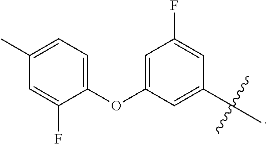 [SMILES: *c1cc(F)cc(Oc2ccc(C)cc2F)c1]

In some embodiments, B is

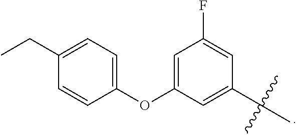 [SMILES: *c1cc(F)cc(Oc2ccc(CC)cc2)c1]

In some embodiments, in which the compound is a compound of Formula (V) or (VA) or a pharmaceutically acceptable salt thereof, X⁴ is C(R^{h})₂. In some embodiments, in which the compound is a compound of Formula (V) or (VA) or a pharmaceutically acceptable salt thereof, X⁴ is CH₂. In some embodiments, B is:

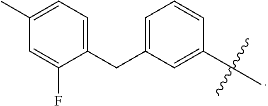 [SMILES: *c1cccc(Cc2ccc(C)cc2F)c1]

In some embodiments, in which the compound is a compound of Formula (V) or (VA) or a pharmaceutically acceptable salt thereof, X⁴ is NR^{h}. In some embodiments, in which the compound is a compound of Formula (V) or (VA) or a pharmaceutically acceptable salt thereof, X⁴ is NH. In some embodiments, B is

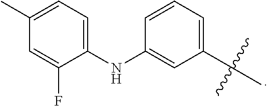 [SMILES: *c1cccc(Nc2ccc(C)cc2F)c1]

In some embodiments, B is optionally substituted C₃-C₁₄ carbocyclyl or optionally substituted 5-to 14-membered heterocyclyl.

In some embodiments, the compound is a compound of Formula (VI):

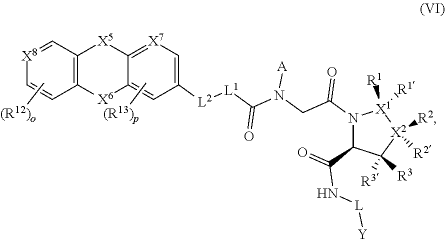

or a pharmaceutically acceptable salt thereof, in which:
each of X⁵ and X⁶ is independently a bond; O; S; C(Rⁱ)₂, in which each Rⁱ is independently H, OH, halo, optionally substituted C₁-C₆ alkyl, or optionally substituted C₁-C₆ alkoxy, or both Rⁱ combine to form oxo; NR^{j}, in which R^{j} is H or C₁-C₆ alkyl; or SO₂;
X⁷ is CH, CR¹³, or N;
X⁸ is CH, CR¹², or N;
o is selected from 0, 1, 2, and 3;
p is selected from 0, 1, and 2;
each R¹² and R¹³ is independently halo, optionally substituted C₁-C₆ alkyl, optionally substituted C₁-C₆ alkoxy, or optionally substituted C₃-C₈ cycloalkyl, and all other variables are as defined for Formula (I).

In some embodiments, the compound is a compound of Formula (VIA):

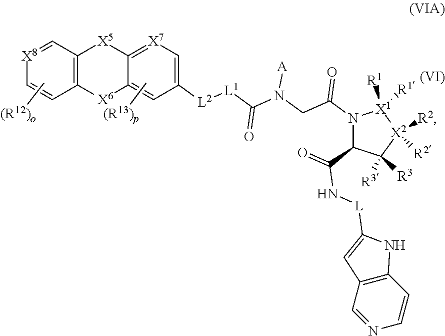

or a pharmaceutically acceptable salt thereof, in which all variables are as defined for Formula (VI).

In some embodiments, the compound is a compound of formula (VII):

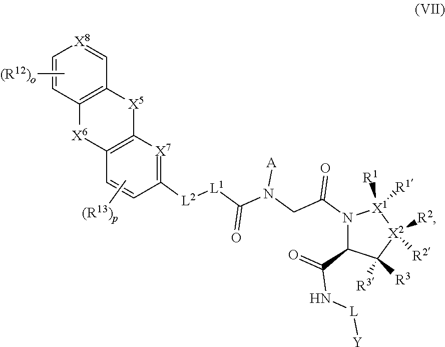

or a pharmaceutically acceptable salt thereof, in which:
each of X⁵ and X⁶ is independently a bond; O; S; C(Rⁱ)₂, in which each Rⁱ is independently H, OH, halo, optionally substituted C₁-C₆ alkyl, or optionally substituted C₁-C₆ alkoxy, or both Rⁱ combine to form oxo; NR^{j}, in which R^{j} is H or C₁-C₆ alkyl; or SO₂;
X⁷ is CH, CR¹³, or N;
X⁸ is CH, CR¹², or N;
o is selected from 0, 1, 2, and 3;
p is selected from 0, 1, and 2; and
each R¹² and R¹³ is independently halo, optionally substituted C₁-C₆ alkyl, optionally substituted C₁-C₆ alkoxy, or optionally substituted C₃-C₈ cycloalkyl, and all other variables are as defined for Formula (I).

In some embodiments, the compound is a compound of Formula (VIIA):

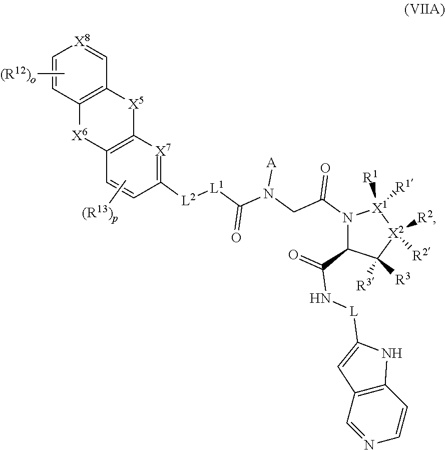

or a pharmaceutically acceptable salt thereof, in which all variables are as defined for Formula (VII).

In some embodiments, X⁵ is C(Rⁱ)₂. In some embodiments, X⁵ is CF₂. In some embodiments, X⁵ is CHCH₃. In some embodiments, X⁵ is C(CH₃)₂. In some embodiments, X⁵ is C(CH₃)(OH). In some embodiments, X⁵ is a bond. In some embodiments, X⁵ is O. In some embodiments, X¹ is S. In some embodiments, X⁵ is NR^{j}. In some embodiments, X⁵ is NH.

In some embodiments, X⁶ is a bond. In some embodiments, X⁶ is O. In some embodiments, X⁶ is S. In some embodiments, X⁶ is C(Rⁱ)₂. In some embodiments, X⁶ is CF₂. In some embodiments, X⁶ is CHCH₃. In some embodiments, X⁶ is C(CH₃)₂. In some embodiments, X⁶ is C(CH₃)(OH). In some embodiments, X⁶ is NR^{j}. In some embodiments, X⁶ is NH.

In some embodiments, X⁷ is CH. In some embodiments, X⁷ is N.

In some embodiments, X⁸ is CH. In some embodiments, X⁸ is N.

In some embodiments, B is

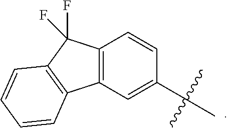 [SMILES: *c1ccc2c(c1)-c1ccccc1C2(F)F]

In some embodiments, B is

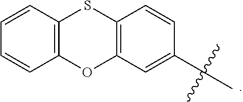 [SMILES: *c1ccc2c(c1)Oc1ccccc1S2]

In some embodiments, B is

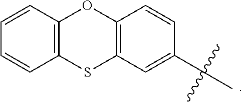 [SMILES: *c1ccc2c(c1)Sc1ccccc1O2]

In some embodiments, B is

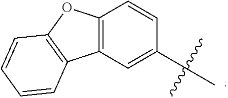 [SMILES: *c1ccc2oc3ccccc3c2c1]

In some embodiments, B is

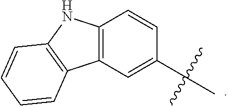 [SMILES: *c1ccc2[nH]c3ccccc3c2c1]

In some embodiments, B is

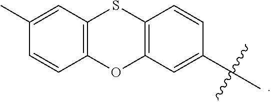 [SMILES: *c1ccc2c(c1)Oc1ccc(C)cc1S2]

In some embodiments, B is

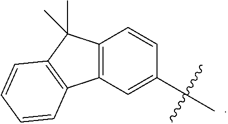 [SMILES: *c1ccc2c(c1)-c1ccccc1C2(C)C]

In some embodiments, B is

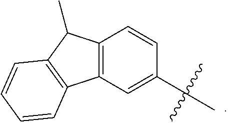 [SMILES: *c1ccc2c(c1)-c1ccccc1C2C]

In some embodiments, B is

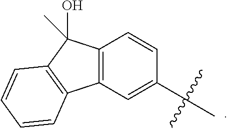 [SMILES: *c1ccc2c(c1)-c1ccccc1C2(C)O]

In some embodiments, B is

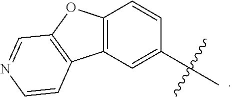 [SMILES: *c1ccc2oc3cnccc3c2c1]

In some embodiments, B is

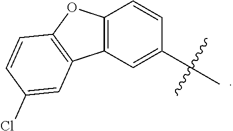 [SMILES: *c1ccc2oc3ccc(Cl)cc3c2c1]

In some embodiments, B is

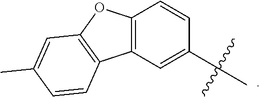 [SMILES: *c1ccc2oc3cc(C)ccc3c2c1]

In some embodiments, B is

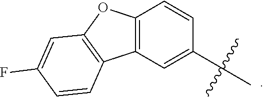 [SMILES: *c1ccc2oc3cc(F)ccc3c2c1]

In some embodiments, B is

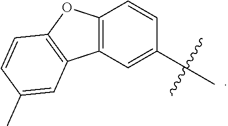 [SMILES: *c1ccc2oc3ccc(C)cc3c2c1]

In some embodiments, B is

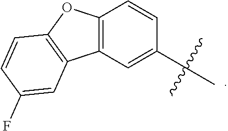 [SMILES: *c1ccc2oc3ccc(F)cc3c2c1]

In some embodiments, B is

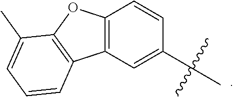 [SMILES: *c1ccc2oc3c(C)cccc3c2c1]

In some embodiments, B is

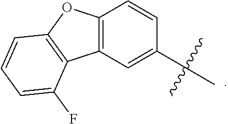 [SMILES: *c1ccc2oc3cccc(F)c3c2c1]

In some embodiments, B is

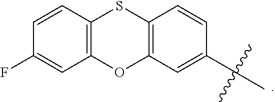 [SMILES: *c1ccc2c(c1)Oc1cc(F)ccc1S2]

In some embodiments, B is

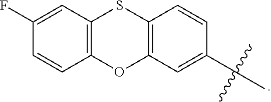 [SMILES: *c1ccc2c(c1)Oc1ccc(F)cc1S2]

In some embodiments, B is

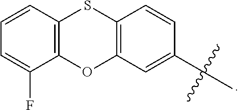 [SMILES: *c1ccc2c(c1)Oc1c(F)cccc1S2]

In some embodiments, B is

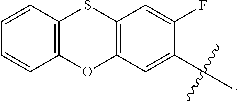 [SMILES: *c1cc2c(cc1F)Sc1ccccc1O2]

In some embodiments, B is

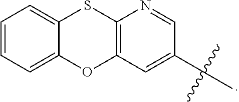 [SMILES: *c1cnc2c(c1)Oc1ccccc1S2]

In some embodiments, B is

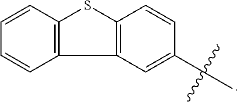 [SMILES: *c1ccc2sc3ccccc3c2c1]

In some embodiments, B is

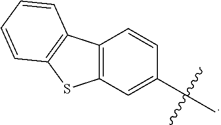 [SMILES: *c1ccc2c(c1)sc1ccccc12]

In some embodiments, B is

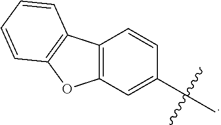 [SMILES: *c1ccc2c(c1)oc1ccccc12]

In some embodiments, B is

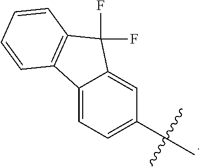 [SMILES: *c1ccc2c(c1)C(F)(F)c1ccccc1-2]

In some embodiments, B is optionally substituted 5-to 10-membered heteroaryl. In some embodiments, B is

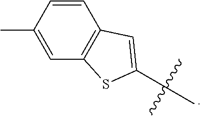 [SMILES: *c1cc2ccc(C)cc2s1]

In some embodiments, B is

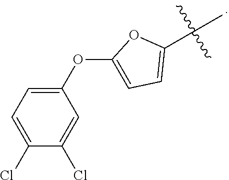 [SMILES: *c1ccc(Oc2ccc(Cl)c(Cl)c2)o1]

In some embodiments, B is

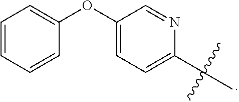 [SMILES: *c1ccc(Oc2ccccc2)cn1]

In some embodiments, B is

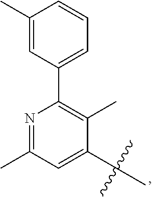 [SMILES: *c1cc(C)nc(-c2cccc(C)c2)c1C]

In some embodiments, B is

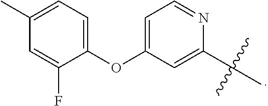 [SMILES: *c1cc(Oc2ccc(C)cc2F)ccn1]

In some embodiments. B is

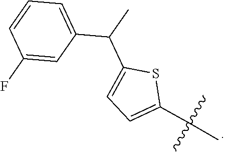 [SMILES: *c1ccc(C(C)c2cccc(F)c2)s1]

In some embodiments, B is

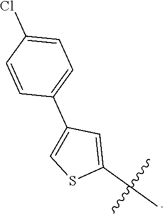 [SMILES: *c1cc(-c2ccc(Cl)cc2)cs1]

In some embodiments, B is

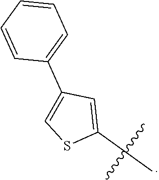 [SMILES: *c1cc(-c2ccccc2)cs1]

In some embodiments, B is

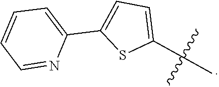 [SMILES: *c1ccc(-c2ccccn2)s1]

In some embodiments, X¹ is C and X² is C.

In some embodiments, R¹ and R², together with the atoms to which each is attached, combine to form optionally substituted C₃-C₈ cycloalkyl. In some embodiments, R¹ and R², together with the atoms to which each is attached, combine to form cyclopropyl.

In some embodiments, R¹ and R², together with the atoms to which each is attached, combine to form optionally substituted C₃-C₈ cycloalkyl. In some embodiments, R^{1′} and R^{2′}, together with the atoms to which each is attached, combine to form cyclopropyl.

In some embodiments, X¹ is N and X² is C.

In some embodiments, R¹ and R² combine to form a double bond.

In some embodiments, R² and R^{2′}, together with the atom to which they are attached, form optionally substituted 4-or 5-membered spirocyclic heterocycle. In some embodiments, R² and R^{2′}, together with the atom to which they are attached, form

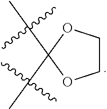 [SMILES: *C1(*)OCCO1]

In some embodiments, R² and R^{2′}, together with the atom to which they are attached, form

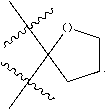 [SMILES: *C1(*)CCCO1]

In some embodiments, R² and R^{2′}, together with the atom to which they are attached, form optionally substituted C₃-C₈ spirocyclic cycloalkyl. In some embodiments, R² and R^{2′}, together with the atom to which they are attached, form spirocyclic cyclopropyl. In some embodiments, R² and R^{2′}, together with the atom to which they are attached, form spirocyclic cyclopentyl.

In some embodiments, R² is H. In some embodiments, R² is optionally substituted C₁-C₆ alkyl. In some embodiments, R² is CH₃. In some embodiments, R² is CH₂F. In some embodiments, R² is CH₂O CH₃. In some embodiments, R² is CH₂O(CH₂)₄NH₂. In some embodiments, R² is CH₂O(CH₂)₄NHC(O)CH₃. In some embodiments, R² is CH₂O(CH₂)₅COOH. In some embodiments, R² is

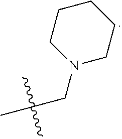 [SMILES: *CN1CCCCC1]

In some embodiments, R² is

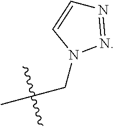 [SMILES: *Cn1ccnn1]

In some embodiments, R² is

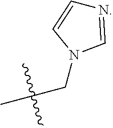 [SMILES: *Cn1ccnc1]

In some embodiments, R² is optionally substituted optionally substituted 5-to 10-membered heteroaryl. In some embodiments, R² is

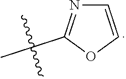 [SMILES: *c1ncco1]

In some embodiments, R² is optionally substituted 5-to 10-membered heterocycle. In some embodiments, R² is

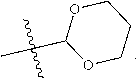 [SMILES: *C1OCCCO1]

In some embodiments, R^{2′} is H. In some embodiments, R^{2′} is halo. In some embodiments, R^{2′} is F. In some embodiments, R^{1′} is optionally substituted C₁-C₆ alkyl. In some embodiments, R^{1′} is CH₃. In some embodiments, R^{1′} is CF₃. In some embodiments, R^{2′} is CH₂OH. In some embodiments, R² is CH₂OCH₃. In some embodiments, R^{2′} is CH₂O(CH₂)₄NH₂. In some embodiments, R^{2′} is CH₂O(CH₂)₅COOH. In some embodiments. R^{2′} is

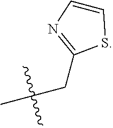 [SMILES: *Cc1nccs1]

In some embodiments, R²′ is

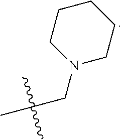 [SMILES: *CN1CCCCC1]

In some embodiments, R^{2′} is

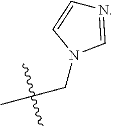 [SMILES: *Cn1ccnc1]

In some embodiments, R^{2′} is

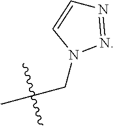 [SMILES: *Cn1ccnn1]

In same embodiments, R^{2′} is

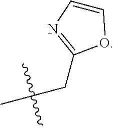 [SMILES: *Cc1ncco1]

In some embodiments, R^{2′} is optionally substituted optionally substituted 5-to 10-membered heteroaryl. In some embodiments, R^{2′} is

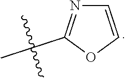 [SMILES: *c1ncco1]

In some embodiments, R^{2′} is

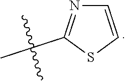 [SMILES: *c1nccs1]

In some embodiments, R^{1′} is optionally substituted 5-to 10-membered heterocycle. In some embodiments, R^{2′} is

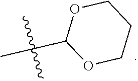 [SMILES: *C1OCCCO1]

In some embodiments, R^{1′} is optionally substituted C₁-C₆ alkoxy. In some embodiments, R^{1′} is OCH₃. In some embodiments, R^{2′} is OCHF₂. In some embodiments, R^{2′} is OCF₃. In some embodiments, R² is SO₂R^{a}. In some embodiments, R^{2′} is SO₂CH. In some embodiments, R^{2′} is SO₂CH(CH₃)₂. In some embodiments, R^{2′} is

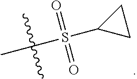 [SMILES: *S(=O)(=O)C1CC1]

In some embodiments, X¹ is C and X² is Si. In some embodiments, in which X¹ is C and X² is Si, R² is optionally substituted C₁-C₆ alkyl. In some embodiments, in which X¹ is C and X² is Si, R² is CH₃. In some embodiments, in which X¹ is C and X² is Si, R^{2′} is optionally substituted C₁-C₆ alkyl. In some embodiments, in which X¹ is C and X² is Si, R^{2′} is CH₃.

In some embodiments, X¹ is C and X² is S.

In some embodiments, R¹ is H. In some embodiments, R¹ is optionally substituted C₁-C₆ alkyl. In some embodiments, R¹ is CH₃.

In some embodiments, R^{1′} is H.

In some embodiments, R³ is H. In some embodiments, R³ is hydroxy.

In some embodiments, R^{3′} is H.

In some embodiments, L is a bond. In some embodiments, L is

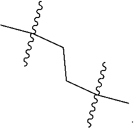 [SMILES: *CC*]

In some embodiments, L is

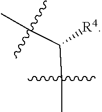

In some embodiments, L is

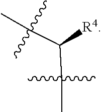

In some embodiments, R⁴ is H. In some embodiments, R⁴ is CH₃. In some embodiments, R⁴ is CF₃. In some embodiments, R⁴ is CH₂OH.

In some embodiments, L¹ is a bond. In some embodiments, L¹ is optionally substituted C₁-C₆ alkylene. In some embodiments, L¹ is —CH₂—. In some embodiments, L¹ is —(CH₂)₃—.

In some embodiments, L² is a bond. In some embodiments, L² is O.

In some embodiments, A is H.

In another aspect, the present disclosure provides a compound of Table 1, or a pharmaceutically acceptable salt thereof.

In some embodiments of any of the aspects described herein (e.g., Formulas (I), (II), (III), (IIIA), (IV), (IVA), (V), (VA), (VI), (VIA), (VII), and (VIIA), and Table 1), a compound of the present disclosure has Complement C₁ esterase (C1s) inhibiting activity.

### Pharmaceutical Compositions

A pharmaceutical composition of the invention contains one or more of the compounds disclosed herein (e.g., one or more of the compounds of formulas (I), (II), (III), (IIIA), (IV), (IVA), (V), (VA), (VI), (VIA), (VII), and (VIIA), and Table 1) as the therapeutic compound. In addition to a therapeutically effective amount of the compound, the pharmaceutical compositions also contain a pharmaceutically acceptable excipient, which can be formulated by methods known to those skilled in the art. In some embodiments, the pharmaceutical compositions for treating cancer contain one or more of the compounds disclosed herein (e.g., one or more of the compounds of formulas (I), (II), (III), (IIIA), (IV), (IVA), (V), (VA), (VI), (VIA), (VII), and (VIIA), and Table 1) may be formulated and/or administered with or without other therapeutics for a particular condition. Examples of such therapeutics (second therapeutic agents) are described herein.

The compounds disclosed herein (e.g., the compounds of formulas (I), (II), (III), (IIIA), (IV), (IVA), (V), (VA), (VI), (VIA), (VII), and (VIIA), and Table 1) may be used in the form of free base or in the form of salts. All forms are within the scope of the disclosure.

Exemplary routes of administration of the pharmaceutical compositions (or the compounds of the composition) include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

### Formulations for Oral Administration

The pharmaceutical compositions of the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers; granulating and disintegrating agents; binding agents; and lubricating agents, glidants, and antiadhesives. Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent, or as soft gelatin capsules where the active ingredient is mixed with water or an oil medium. Powders, granulates, and pellets may be prepared using the ingredients mentioned above under tablets and capsules in a conventional manner using, e.g., a mixer, a fluid bed apparatus or a spray drying equipment.

The liquid forms in which the compounds and compositions of the present invention can be incorporated for administration orally include aqueous solutions, suitably flavored syrups, aqueous or oil suspensions, and flavored emulsions with edible oils, as well as elixirs and similar pharmaceutical vehicles.

### Formulations for Parenteral Administration

The pharmaceutical compositions of the invention can be administered in a pharmaceutically acceptable parenteral (e.g., intravenous, intramuscular, subcutaneous or the like) formulation as described herein. The pharmaceutical composition may also be administered parenterally in dosage forms or formulations containing conventional, non-toxic pharmaceutically acceptable carriers and adjuvants. In particular, formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water; water adjusted to a suitable pH by addition of an appropriate amount of hydrochloric acid, sodium hydroxide, or a suitable buffer; 1,3-butanediol; Ringer's solution; and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), incorporated by reference herein in its entirety.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropyl cellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 23^{rd} Ed., Adejare, Ed., Academic Press (2020) and in The United States Pharmacopeia and National Formulary (USP-NF 2021 Issues 1-3), published in 2021.

Formulations for parenteral administration may, for example, contain sterile water, saline, polyalkylene glycols (e.g., polyethylene glycol), oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain, for example, lactose, or may be aqueous solutions containing, for example, polyoxyethylene-9-lauryl ether, glycocholate and deoxycholate, or may be oily solutions for administration in the form of nasal drops, or as a gel.

The dosage of the compounds described herein (e.g., the compounds of formulas (I) and Table 1), and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the subject to be treated. One of skill in the art can determine the appropriate dosage based on the above factors. In general, satisfactory results may be obtained when the compounds described herein are administered to a human at a daily dosage of, for example, between 0.05 mg and 3000 mg (measured as the solid form). For example, the dose range may be 10-1000 mg (e.g., 50-800 mg).

Alternatively, the dosage amount can be calculated using the body weight of the patient. For example, the dose of a compound, or pharmaceutical composition thereof, administered to a patient may be 0.1-100 mg/kg. A dosage form containing a compound disclosed herein (e.g., a compound of formula (I), (II), (III), (IIIA), (IV), (IVA), (V), (VA), (VI), (VIA), (VII), or (VIIA), or Table 1) can be administered, for example, once a day (QD), twice a day (BID), three times a day (TID), four times a day (QID), once every other day (Q2D), once every third day (Q3D), or any dosing schedule as needed,

### Uses of Active Compounds for Treatment of Selected Disorders

In one aspect, an effective amount of a compound described herein (e.g., any one of the compounds of formulas (I), (II), (III), (IIIA), (IV), (IVA), (V), (VA), (VI), (VIA), (VII), and (VIIA), and Table 1, or a pharmaceutically acceptable salt thereof) is used to treat a medical disorder which is an inflammatory or immune condition, a disorder mediated by the complement cascade (including a dysfunctional cascade) including a complement-related disorder or alternative complement pathway-related disorder, a disorder or abnormality of a cell that adversely affects the ability of the cell to engage in or respond to normal complement activity, or an undesired complement-mediated response to a medical treatment, such as surgery or other medical procedure or a pharmaceutical or biopharmaceutical drug administration, a blood transfusion, or other allogenic tissue or fluid administration.

In some embodiments, the disorder is an autoimmune disease. In some embodiments, the disorder is cancer. In some embodiments, the disorder is an infectious disease. In some embodiments, the disorder is an inflammatory disease. In some embodiments, the disorder is a hematological disease. In some embodiments, the disorder is an ischemia-reperfusion injury. In some embodiments, the disorder is an ocular disease. In some embodiments, the disorder is a renal disease. In some embodiments, the disorder is transplant rejection. In some embodiments, the disorder is antibody-mediated transplant rejection, e.g., acute antibody-mediated rejection. In some embodiments, the disorder is a vascular disease. In some embodiments, the disorder is a vasculitis disorder. In some embodiments, the disorder is a neurodegenerative disorder, e.g., a tauopathy.

In some embodiments, the disorder is a medical disorder of the central nervous system (CNS) or peripheral nervous system disorders involving complement activation. In some embodiments, the disorder is an acquired brain or spinal cord injury. In some embodiments, the disorder is ischemic-reperfusion injury. In some embodiments, the disorder is stroke. In some embodiments, the disorder is traumatic brain injury (TBI). In some embodiments, the disorder is spinal cord injury (SCI).

In some embodiments, the disorder is a neuroinflammatory disorder.

In some embodiments, the neuroinflammatory disorder is cranial arteritis. In some embodiments, the neuroinflammatory disorder is giant cell arteritis. In some embodiments, the neuroinflammatory disorder is Holmes-Adie syndrome. In some embodiments, the neuroinflammatory disorder is inclusion body myositis (IBM). In some embodiments, the neuroinflammatory disorder is meningitis. In some embodiments, the neuroinflammatory disorder is a neurologic paraneoplastic syndrome, e.g., Lambert-Eaton myasthenic syndrome, stiff-person syndrome, encephalomyelitis (inflammation of the brain and spinal cord), myasthenia gravis, cerebellar degeneration, limbic and/or brainstem encephalitis, neuromyotonia, opsoclonus (involving eye movement), or sensory neuropathy. In some embodiments, the neuroinflammatory disorder is polymyositis. In some embodiments, the neuroinflammatory disorder is transverse myelitis. In some embodiments, the neuroinflammatory disorder is vasculitis, e.g., temporal arteritis. In some embodiments, the neuroinflammatory disorder is arachnoiditis. In some embodiments, the neuroinflammatory disorder is Kinsbourne syndrome. In some embodiments, the neuroinflammatory disorder is opsoclonus myoclonus syndrome (OMS). In some embodiments, the neuroinflammatory disorder is Saint Vitus Dance or Sydenham chorea (SD) disease.

In some embodiments, the disorder is Alzheimer's disease (AD). AD is characterized by two hallmark pathologies; amyloid-β (Aβ) plaques and neurofibrillary tangles comprising hyperphosphorylated tau. Recent studies have implicated complement in AD pathogenesis, including genome-wide association studies identifying single nucleotide polymorphisms (SNPs) associated with risk of late-onset AD in genes encoding complement proteins Clusterin (CLU) and CR1 (CR1). See Carpanini et al., Therapeutic Inhibition of the Complement System in Diseases of the Central Nervous System, Front. Immunol., 4 Mar. 2019. Biomarker studies have also identified complement proteins and activation products in plasma and/or CSF that distinguish AD from controls and predict risk of progression to AD.

In some embodiments, the disorder is frontotemporal dementia. In some embodiments, the disorder is Pick's disease. In some embodiments, the disorder is sporadic frontotemporal dementia, e.g., frontotemporal dementia with Parkinsonism linked to chromosome 17. In some embodiments, progressive supranuclear palsy (PSP). In some embodiments, corticobasal degeneration (CBD). In some embodiments, the disorder is subacute sclerosing panencephalitis.

In some embodiments, the disorder is amyotrophic lateral sclerosis (ALS). ALS is caused by progressive loss of upper and lower (a) motor neurons resulting in denervation of neuromuscular junctions in the peripheral nervous system, progressive muscle weakness, atrophy, spasticity, respiratory failure, and ultimately paralysis and death. Recent studies have shown increased C1q protein in motor cortex and spinal cord of ALS post-mortem tissue; C3 activation fragments and TCC in areas of pathology; C4d and TCC staining of degenerating neurons and glia in ALS motor cortex and spinal cord, and C5aR1 upregulation in areas of pathology. C3d and C4d have been found on oligodendroglia and degenerating neurites, surrounded by CR4-positive microglia, in spinal cord and motor cortex, and C1q, C3, and TCC have been shown to be present on motor endplates in intercostal muscles in ALS donors even early in the disease process. See Carpanini et al., Therapeutic Inhibition of the Complement System in Diseases of the Central Nervous System, Front. Immunol., 4 Mar. 2019.

In some embodiments, the disorder is Parkinson's disease (PD). PD is characterized by loss of dopaminergic neurons in the substantia nigra and deposits of the protein α-synuclein that form the pathological hallmarks of the disease, Lewy bodies. Patients present with resting tremor, bradykinesia, and rigidity. Complement activation has been associated with α-synuclein and Lewy bodies in Parkinson's disease; in vitro studies have demonstrated that the disease-associated splice variant α-synuclein 112, but not the full-length protein, cause activation of complement. In vivo, C3d, C4d, C7 and C9 localization in Lewy bodies has been reported. More recently, deposition of iC3b and C9 in Lewy bodies and melanized neurons has been reported, and iC3b immunoreactivity has been shown to be increased with normal ageing and was further elevated in PD vs. age-matched controls. Furthermore, correlation between the ratios of C3/Aβ42 or FH/Aβ42 in CSF and severity of Parkinson's disease motor and cognitive symptoms has been shown. See Carpanini et al., Therapeutic Inhibition of the Complement System in Diseases of the Central Nervous System, Front. Immunol., 4 Mar. 2019. In some embodiments, the subject to be treated suffers from Parkinson's Disease with dementia (PDD).

In some embodiments, the disorder is Huntington's disease (HD). HD is an autosomal dominant, inherited neurodegenerative disease characterized by progressive motor symptoms, psychiatric disturbances, and dementia. It is caused by expansion of a three-base-pair (CAG) repeat (39-121 repeats vs. normal range 8-39 repeats) in exon 1 of the HTT gene that translates into a polyglutamine tract at the N-terminus of the protein. This results in a polyglutamine length-dependent misfolding and accumulation of huntingtin protein in the striatum and cortex (layers 3, 5, and 6) followed by neuronal loss in these areas which spreads to the hippocampus. It has been shown that neurons, astrocytes, and myelin sheaths in the HD caudate and striatum were immunoreactive for C1q, C4, C3 and neo-epitopes in iC3b and TCC. Expression of mRNA encoding early complement components C1q (c-chain), C1r, C3, and C4, complement regulators ClINII, Clusterin, MCP, DAF and CD59, and complement receptors C3a and C5a, have been shown to be upregulated in the HD striatum, see Carpanini et al., Therapeutic Inhibition of the Complement System in Diseases of the Central Nervous System, Front. Immunol., 4 Mar. 2019.

In some embodiments, the disorder is argyrophilic grain dementia. In some embodiments, the disorder is British type amyloid angiopathy. In some embodiments, the disorder is cerebral amyloid angiopathy. In some embodiments, the disorder is Creutzfeldt-Jakob disease. In some embodiments, the disorder is dementia pugilistica. In some embodiments, the disorder is diffuse neurofibrillary tangles with calcification. In some embodiments, the disorder is Down's syndrome. In some embodiments, the disorder is frontotemporal lobar degeneration. In some embodiments, the disorder is Gerstmann-Straussler-Scheinker disease. In some embodiments, the disorder is Hallervorden-Spatz disease. In some embodiments, the disorder is inclusion body myositis. In some embodiments, the disorder is multiple system atrophy (MSA). In some embodiments, the disorder is myotonic dystrophy. In some embodiments, the disorder is Niemann-Pick disease type C. In some embodiments, the disorder is non-Guamanian motor neuron disease with neurofibrillary tangles. In some embodiments, the disorder is postencephalitic parkinsonism. In some embodiments, the disorder is prion protein cerebral amyloid angiopathy. In some embodiments, the disorder is progressive subcortical gliosis. In some embodiments, the disorder is progressive supranuclear palsy. In some embodiments, the disorder is subacute sclerosing panencephalitis. In some embodiments, the disorder is Tangle only dementia. In some embodiments, the disorder is multi-infarct dementia. In some embodiments, the disorder is ischemic stroke. In some embodiments, the disorder is chronic traumatic encephalopathy (CTE).

In some embodiments, the disorder is a hereditary motor and sensory neuropathy (HMSN). In some embodiments, the HMSN is Charcot-Marie-Tooth (CMT) disease. In some embodiments, the HSMN is Charcot-Marie-Tooth disease type 1A or type 1B. In some embodiments, the HSMN is Charcot-Marie-Tooth disease type 2. In some embodiments, the HSMN is Dejerine-Sottas disease (Charcot-Marie-Tooth type 3). In some embodiments, the HSMN is Refsum disease. In some embodiments, the HSMN is Charcot-Marie-Tooth with pyramidal features. In some embodiments, the HSMN is Charcot-Marie-Tooth type 6. In some embodiments, the HSMN is HMSN+retinitis pigmentosa.

In some embodiments, the disorder is Churg-Strauss syndrome. In some embodiments, the disorder is peripheral artery disease (PAD). In some embodiments, the disorder is myasthenia gravis, e.g., myasthenia gravis with CNS involvement. In some embodiments, the disorder is dementia with Lewy bodies. In some embodiments, the disorder is prion disease. In some embodiments, the disorder is Behcet's Disease. In some embodiments, the disorder is congenital myasthenia. In some embodiments, the disorder is subacute sclerosing panencephalitis (SSPE).

In some embodiments, the disorder is a demyelinating disease. In some embodiments, the disorder is demyelinating myelinoclastic disease. In some embodiments, the disorder is demyelinating leukodystrophic disease.

In some embodiments, the demyelinating myelinoclastic disease is multiple sclerosis (MS). Multiple sclerosis (MS) is the most common cause of neurological disability in young adults in northern European-Caucasian populations, with an approximate lifetime risk of one in 400. C3 has been shown to be deposited in the brains of MS patients. T-cell clone (TCC) has been shown to be in association with capillary endothelial cells, predominantly within plaques and adjacent white matter. Localization of C activation to areas of active myelin destruction has also been shown, with TCC deposited exclusively in such areas. C3d has been shown to be deposited in association with short segments of disrupted myelin in plaques with low-grade active demyelination and provides evidence for a C contribution to disease progression as well as acute inflammation. See Ingram et al, Complement in multiple sclerosis: its role in disease and potential as a biomarker. Clin Exp Immunol. 2009 February; 155(2):128-39.

In some embodiments, the demyelinating myelinoclastic disease is neuromyelitis optica (NMO). Neuromyelitis optica (NMO) is an inflammatory demyelinating disease affecting predominantly the optic nerves and spinal cord. Traditionally seen as a variant of MS, it has been redefined recently according to new criteria using a combination of phenotypic subtyping along with a newly developed biomarker of disease, NMO-immunoglobulin G (IgG) (reported sensitivity of 58-76% and specificity of 85-99% for NMO). NMO patients have higher levels of C3a and anti-C1q antibodies than healthy controls. C3a levels correlated with disease activity, neurological disability and aquaporin-4 IgG. Nytrova et al. J Neuroimmunol. 2014 Sep. 15; 274(1-2):185-91.

In some embodiments, the demyelinating myelinoclastic disease is neuromyelitis optica spectrum disorder (NMOSD). In some embodiments, the demyelinating myelinoclastic disease is idiopathic inflammatory demyelinating diseases (IIDD). In some embodiments, the demyelinating myelinoclastic disease is anti-NMDA receptor encephalitis. In some embodiments, the demyelinating myelinoclastic disease is acute disseminated encephalomyelitis. In some embodiments, the demyelinating myelinoclastic disease is anti-MOG autoimmune encephalomyelitis. In some embodiments, the demyelinating myelinoclastic disease is chronic relapsing inflammatory optic neuritis (CRION). In some embodiments, the demyelinating myelinoclastic disease is acute disseminated encephalomyelitis (ADEM). In some embodiments, the demyelinating myelinoclastic disease is immune-mediated encephalomyelitis. In some embodiments, the demyelinating myelinoclastic disease is progressive multifocal leukoencephalopathy (PML). In some embodiments, the demyelinating myelinoclastic disease is McDonalds-positive multiple sclerosis. In some embodiments, the demyelinating myelinoclastic disease is acute hemorrhagic leukoencephalitis. In some embodiments, the demyelinating myelinoclastic disease is Rasmussen's Encephalitis. In some embodiments, the demyelinating myelinoclastic disease is Marburg multiple sclerosis. In some embodiments, the demyelinating myelinoclastic disease is pseudotumefactive or tumefactive multiple sclerosis. In some embodiments, the demyelinating myelinoclastic disease is Balo concentric sclerosis. In some embodiments, the demyelinating myelinoclastic disease is diffuse myelinoclastic sclerosis. In some embodiments, the demyelinating myelinoclastic disease is solitary sclerosis. In some embodiments, the demyelinating myelinoclastic disease is multiple sclerosis with cavitary lesions. In some embodiments, the demyelinating myelinoclastic disease is myelocortical multiple sclerosis (MCMS). In some embodiments, the demyelinating myelinoclastic disease is atypical optic-spinal multiple sclerosis. In some embodiments, the demyelinating myelinoclastic disease is pure spinal multiple sclerosis. In some embodiments, the demyelinating myelinoclastic disease is HLA DRB3*02:02 multiple sclerosis. In some embodiments, the demyelinating myelinoclastic disease is autoimmune GFAP astrocytopathy. In some embodiments, the demyelinating myelinoclastic disease is chronic inflammatory demyelinating polyneuropathy (CIDP). In some embodiments, the demyelinating myelinoclastic disease is Guillain-Barre syndrome (acute or chronic). In some embodiments, the demyelinating myelinoclastic disease is progressive inflammatory neuropathy. In some embodiments, the demyelinating myelinoclastic disease is Lewis-Sumner Syndrome. In some embodiments, the demyelinating myelinoclastic disease is combined central and peripheral demyelination (CCPD). In some embodiments, the demyelinating myelinoclastic disease is Bickerstaff brainstem encephalitis. In some embodiments, the demyelinating myelinoclastic disease is Fisher syndrome. In some embodiments, the demyelinating myelinoclastic disease is trigeminal neuralgia. In some embodiments, the demyelinating myelinoclastic disease is NMDAR anti-NMDA receptor encephalitis. In some embodiments, the demyelinating myelinoclastic disease is primary progressive MS (PPMS). In some embodiments, the demyelinating myelinoclastic disease is OPA1 variant multiple sclerosis. In some embodiments, the demyelinating myelinoclastic disease is KIR4.1 multiple sclerosis. In some embodiments, the demyelinating myelinoclastic disease is aquaporine-related multiple sclerosis. In some embodiments, the demyelinating myelinoclastic disease is chronic cerebrospinal venous insufficiency (CCSVI or CCVI). In some embodiments, the demyelinating myelinoclastic disease is diffuse sclerosis. In some embodiments, the demyelinating myelinoclastic disease is Schilder's disease.

In certain aspects, the disorder to be treated is a demyelinating leukodystrophic disease. In some embodiments, the demyelinating leukodystrophic disease is myelitis. In some embodiments, the demyelinating leukodystrophic disease is central pontine myelinolysis (CPM). In some embodiments, the demyelinating leukodystrophic disease is extrapontine myelinolysis. In some embodiments, the demyelinating leukodystrophic disease is tabes dorsalis. In some embodiments, the demyelinating leukodystrophic disease is progressive multifocal leukoencephalopathy. In some embodiments, the demyelinating leukodystrophic disease is leukoencephalopathy with vanishing white matter. In some embodiments, the demyelinating leukodystrophic disease is leukoencephalopathy with neuroaxonal spheroids. In some embodiments, the demyelinating leukodystrophic disease is reversible posterior leukoencephalopathy syndrome. In some embodiments, the demyelinating leukodystrophic disease is megalencephalic leukoencephalopathy with subcortical cysts. In some embodiments, the demyelinating leukodystrophic disease is megalencephalic leukoencephalopathy with subcortical cysts 1. In some embodiments, the demyelinating leukodystrophic disease is hypertensive leukoencephalopathy. In some embodiments, the demyelinating leukodystrophic disease is metachromatic leukodystrophy. In some embodiments, the demyelinating leukodystrophic disease is Krabbe disease. In some embodiments, the demyelinating leukodystrophic disease is Canavan disease. In some embodiments, the demyelinating leukodystrophic disease is X-linked adrenoleukodystrophy. In some embodiments, the demyelinating leukodystrophic disease is Alexander disease. In some embodiments, the demyelinating leukodystrophic disease is cerebrotendinous xanthomatosis. In some embodiments, the demyelinating leukodystrophic disease is Pelizaeus-Merzbacher disease. In some embodiments, the demyelinating leukodystrophic disease is Refsum disease.

In some embodiments, an effective amount of a compound described herein, or a pharmaceutically acceptable salt thereof, is used to treat Buerger's disease, also known as thromboangiitis obliterans.

In some embodiments, an effective amount of a compound described herein, or a pharmaceutically acceptable salt thereof, is used to treat giant cell arteritis.

In some embodiments, an effective amount of a compound described herein, or a pharmaceutically acceptable salt thereof, is used to treat Raynaud's disease.

In certain aspects, the disorder to be treated is a demyelinating disease of the peripheral nervous system. In some embodiments, the demyelinating disease of the peripheral nervous system is anti-MAG peripheral neuropathy. In some embodiments, the demyelinating disease of the peripheral nervous system is hereditary neuropathy with liability to pressure palsy. In some embodiments, the demyelinating disease of the peripheral nervous system is a copper deficiency-associated condition (e.g., peripheral neuropathy, myelopathy, or rarely optic neuropathy).

In some embodiments, an effective amount of a compound described herein, or a pharmaceutically acceptable salt thereof, is used to treat transverse myelitis.

In certain aspects, the disorder to be treated is a peripheral neuropathy. In some embodiments, the peripheral neuropathy is a mononeuropathy. In some embodiments, the neuropathy is a polyneuropathy. In some embodiments, the polyneuropathy is distal axonopathy, diabetic neuropathy, a demyelinating polyneuropathy, small fiber peripheral neuropathy, mononeuritis multiplex, polyneuritis multiplex, autonomic neuropathy, or neuritis.

In some embodiments, an effective amount of a compound described herein, or a pharmaceutically acceptable salt thereof, is used to treat multifocal motor neuropathy.

In some embodiments, an effective amount of a compound described herein, or a pharmaceutically acceptable salt thereof, is used to treat an autoimmune vascular disease. In some embodiments, the autoimmune vascular disease is vasculitis. In some embodiments, the vasculitis includes, but is not limited to, autoimmune inflammatory vasculitis, Cutaneous small-vessel vasculitis, Granulomatosis with polyangiitis, Eosinophilic granulomatosis with polyangiitis, Behget's disease, Kawasaki disease, Buerger's disease, and “Limited” granulomatosis with polyangiitis vasculitis.

In some embodiments, a compound (e.g., a compound of formula (I), (II), (III), (IIIA), (IV), (IVA), (V), (VA), (VI), (VIA), (VII), or (VIIA), or Table 1) or its salt or composition as described herein is used to treat an arteritis. In some embodiments, the arteritis is giant cell arteritis. In some embodiments, the arteritis is Takayasu arteritis. In some embodiments, the arteritis is temporal arteritis. In some embodiments, the arteritis is polyarteritis nodosa.

In some embodiments, a method for the treatment of a glomerulonephritis is provided. In some embodiment, the glomerulonephritis is membranoproliferative glomerulonephritis (MPGN). In some embodiments, the MPGN is MPGN Type I. In some embodiments, the MPGN is MPGN Type II. In some embodiments, the MPGN is MPGN Type III. In some embodiments, the MPGN is C3 glomerulonephritis (C3G). In some embodiments, the MPGN is dense deposit disease (DDD). In some embodiments, the MPGN is a C4 deposition disorder.

In some embodiments, the glomerulonephritis is IC-MPGN. In some embodiments, the glomerulonephritis is a membranous glomerulonephritis. In some embodiments, the glomerulonephritis is IgA nephropathy. In some embodiments, the glomerulonephritis is post-infectious glomemlonephritis. In some embodiments, the glomenilonephritis is a rapidly progressive glomerulonephritis, for example Type I (Goodpasture syndrome), Type II, or Type III rapidly progressive glomerulonephritis.

In some embodiments, a method for the treatment of paroxysmal nocturnal hemoglobinuria (PNH) is provided that includes the administration of an effective amount of a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof to a subject, optionally in a pharmaceutically acceptable composition.

In some embodiments, a method for the treatment of hereditary angioedema (HAE) is provided that includes the administration of an effective amount of a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof to a subject, optionally in a pharmaceutically acceptable composition. Mutations in the SERPING1 gene cause hereditary angioedema type I and type II. Hereditary angioedema is a disorder characterized by recurrent episodes of severe swelling (angioedema). The most common areas of the body to develop swelling are the limbs, face, intestinal tract, and airway. The SERPING1 gene provides instructions for making the C1 inhibitor protein, which is important for controlling inflammation. C1 inhibitor blocks the activity of certain proteins that promote inflammation. Mutations that cause hereditary angioedema type I lead to reduced levels of C1 inhibitor in the blood, while mutations that cause type II result in the production of a C1 inhibitor that functions abnormally. Without the proper levels of functional C1 inhibitor, excessive amounts of a protein fragment (peptide) called bradykinin are generated. Bradykinin promotes inflammation by increasing the leakage of fluid through the walls of blood vessels into body tissues. Excessive accumulation of fluids in body tissues causes the episodes of swelling seen in individuals with hereditary angioedema type I and type II.

In some embodiments, a method for the treatment of cold agglutinin disease (CAD) is provided that includes the administration of an effective amount of a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof to a subject, optionally in a pharmaceutically acceptable composition. CAD is a rare autoimmune hemolytic condition with potentially serious acute and chronic consequences that are driven by C1 activation of the classical complement pathway.

In some embodiments, a method for the treatment of atypical hemolytic uremic syndrome (aHUS) is provided that includes the administration of an effective amount of a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof to a subject, optionally in a pharmaceutically acceptable composition. Atypical hemolytic-uremic syndrome is a disease that primarily affects kidney function. Atypical hemolytic uremic syndrome, which can occur at any age, causes abnormal blood clots (thrombi) to form in small blood vessels in the kidneys. These clots can cause serious medical problems if they restrict or block blood flow. Atypical hemolytic-uremic syndrome is characterized by three major features related to abnormal clotting: hemolytic anemia, thrombocytopenia, and kidney failure.

In another embodiment, a method for the treatment of wet or dry age-related macular degeneration (AMD) in a subject is provided that includes the administration of an effective amount of a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof to a subject, optionally in a pharmaceutically acceptable composition. In another embodiment, a method for the treatment of rheumatoid arthritis in a subject is provided that includes the administration of an effective amount of a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof to a subject, optionally in a pharmaceutically acceptable composition.

In another embodiment, a method for the treatment of multiple sclerosis in a subject is provided that includes the administration of an effective amount of a compound disclosed herein (e.g., any one of the compounds of formulas (I), (II), (III), (IIIA), (IV), (IVA), (V), (VA), (VI), (VIA), (VII), and (VIIA), and Table 1) or a pharmaceutically acceptable salt thereof to a subject, optionally in a pharmaceutically acceptable composition.

The active compounds or pharmaceutically acceptable salts thereof disclosed herein, are also useful for administration in combination (in the same or a different dosage form) or alternation with a second pharmaceutical agent for use in ameliorating or reducing a side effect of the second pharmaceutical agent.

For example, in some embodiments, the active compound may be used in combination with an adoptive cell-transfer therapy to reduce an inflammatory response associated with such therapy, for example, a cytokine mediated response such as cytokine response syndrome.

In some embodiments, the adoptive cell-transfer therapy is a chimeric antigen receptor T-Cell (CAR T), or a dendritic cell used to treat a hematologic or solid tumor, for example, a B-cell related hematologic cancer.

In some embodiments, the hematologic or solid tumor is acute lymphoblastic leukemia (ALL), acute myeloid leukemia (AML), non-Hodgkin's lymphoma, chronic lymphocytic leukemia (CLL), pancreatic cancer, glioblastoma, or a cancer that expresses CD19.

In some embodiments, the adoptive cell-transfer therapy is a non-engineered T-cell therapy, in which the T-cells have been activated and/or expanded to one or more viral or tumor antigens. In some embodiments, the associated inflammatory response is a cytokine mediated response.

In some embodiments, the second pharmaceutical agent is a cell that has been transformed to express a protein, in which the protein in the subject is mutated or otherwise has impaired function. In some embodiments, the transformed cell includes a CRISPR gene.

Another embodiment is provided that includes the administration of an effective amount of a compound (e.g., a compound of formula (I), (II), (III), (IIIA), (IV), (IVA), (V), (VA), (VI), (VIA), (VII), or (VIIA), or Table 1) or a pharmaceutically acceptable salt thereof, optionally in a pharmaceutically acceptable composition to a subject to treat an ocular, pulmonary, gastrointestinal, or other disorder.

In other embodiments of the disclosure, a compound (e.g., a compound of formula (I), (II), (III), (IIIA), (IV), (IVA), (V), (VA), (VI), (VIA), (VII), or (VIIA), or Table 1) provided herein can be used to treat or prevent a disorder in a subject mediated by complement. As examples, the disclosure includes methods to treat or prevent complement associated disorders that are induced by antibody-antigen interactions, a component of an immune or autoimmune disorder or by ischemic injury. The disclosure also provides methods to decrease inflammation or an immune response, including an autoimmune response, where mediated or affected by the classical complement pathway.

In some embodiments, the disorder is selected from fatty liver and conditions stemming from fatty liver, such as nonalcoholic steatohepatitis (NASH), liver inflammation, cirrhosis, and liver failure. In some embodiments of the present disclosure, a method is provided for treating fatty liver disease in a subject by administering an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In another embodiment, a compound (e.g., a compound of formula (I), (II), (III), (IIIA), (IV), (IVA), (V), (VA), (VI), (VIA), (VII), or (VIIA), or Table 1) or its salt or composition as described herein is used to modulate an immune response prior to or during surgery or other medical procedure. One non-limiting example is use in connection with acute or chronic graft versus subject disease, which is a common complication as a result of organ transplantation, allogeneic tissue transplant, and can also occur as a result of a blood transfusion.

In some embodiments, the present disclosure provides a method of treating dermatomyositis by administering to a subject in need thereof an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, the present disclosure provides a method of treating amyotrophic lateral sclerosis by administering to a subject in need thereof an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, the present disclosure provides a method of treating abdominal aortic aneurysm, hemodialysis complications, hemolytic anemia, or hemodialysis by administering to a subject in need thereof an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In another embodiment, a method is provided for the treatment or prevention of cytokine or inflammatory reactions in response to the administration of pharmaceutical or biotherapeutic (e.g., CAR T-cell therapy or monoclonal antibody therapy) in a subject by administering an effective amount of a compound (e.g., a compound of formula (I), (II), (III), (IIIA), (IV), (IVA), (V), (VA), (VI), (VIA), (VII), or (VIIA), or Table 1) or its salt or composition as described herein. Various types of cytokine or inflammatory reactions may occur in response to a number of factors, such as the administrations of biotherapeutics.

In some embodiments, the cytokine or inflammatory reaction is cytokine release syndrome. In some embodiments, the cytokine or inflammatory reaction is tumor lysis syndrome (which also leads to cytokine release). Symptoms of cytokine release syndrome range from fever, headache, and skin rashes to bronchospasm, hypotension, and even cardiac arrest. Severe cytokine release syndrome is described as a cytokine storm and can be fatal.

Fatal cytokine storms have been observed in response to infusion with several monoclonal antibody therapeutics. See, Abramowicz D, et al. “Release of tumor necrosis factor, interleukin-2, and gamma-interferon in serum after injection of OKT3 monoclonal antibody in kidney transplant recipients” Transplantation (1989) 47(4):606-8; Chatenoud L, et al. “In vivo cell activation following OKT3 administration. Systemic cytokine release and modulation by corticosteroids” Transplantation (1990) 49(4):697-702; and Lim L C, Koh L P, and Tan P. “Fatal cytokine release syndrome with chimeric anti-CD20 monoclonal antibody rituximab in a 71-year-old patient with chronic lymphocytic leukemia” J. Clin Oncol. (1999) 17(6):1962-3.

Also contemplated herein, is the use of a compound (e.g., a compound of formula (I), (II), (III), (IIIA), (IV), (IVA), (V), (VA), (VI), (VIA), (VII), or (VIIA), or Table 1) or its salt or composition as described herein to mediate an adverse immune response in patients receiving bi-specific T-cell engagers (BiTE). A bi-specific T-cell engager directs T-cells to target and bind with a specific antigen on the surface of a cancer cell. For example, Blinatumomab (Amgen), a BiTE has recently been approved as a second line therapy in Philadelphia chromosome-negative relapsed or refractory acute lymphoblastic leukemia. Blinatumomab is given by continuous intravenous infusion in 4-week cycles. The use of BiTE agents has been associated with adverse immune responses, including cytokine release syndrome. The most significantly elevated cytokines in the CRS associated with ACT include IL-10, IL-6, and IFN-γ(Klinger et al., Immunopharmacologic response of patients with B-lineage acute lymphoblastic leukemia to continuous infusion of T cell-engaging CD19/CD3-bispecific BiTE antibody blinatumomab. Blood (2012) 119:6226-6233).

In another embodiment, the disorder is episcleritis, idiopathic episcleritis, anterior episcleritis, or posterior episcleritis. In some embodiments, the disorder is idiopathic anterior uveitis, HLA-B27 related uveitis, herpetic keratouveitis, Posner Schlossman syndrome, Fuch's heterochromic iridocyclitis, or cytomegalovims anterior uveitis.

In some embodiments, the present disclosure provides a method of treating an IC-MPGN by administering to a subject in need thereof an effective amount of a compound (e.g., a compound of formula (I), (II), (III), (IHA), (IV), (IVA), (V), (VA), (VI), (VIA), (VII), or (VILA), or Table 1) or its salt or composition as described herein.

In some embodiments, the present disclosure provides a method of treating a paroxysmal nocturnal hemoglobinuria (PNH) by administering to a subject in need thereof an effective amount of a compound (e.g., a compound of formula (I), (II), (III), (IIA), (IV), (IVA), (V), (VA), (VI), (VIA), (VII), or (VIIA), or Table 1) or its salt or composition as described herein.

In some embodiments, the present disclosure provides a method of treating a hereditary angioedema (HAE) by administering to a subject in need thereof an effective amount of a compound (e.g., a compound of formula (I), (II), (III), (IIIA), (IV), (IVA), (V), (VA), (VI), (VIA), (VII), or (VILA), or Table 1) or its salt or composition as described herein.

In some embodiments, the present disclosure provides a method of treating cold agglutinin disease (CAD) by administering to a subject in need thereof an effective amount of a compound (e.g., a compound of formula (I), (II), (III), (IIA), (IV), (IVA), (V), (VA), (VI), (VIA), (VII), or (VIIA), or Table 1) or its salt or composition as described herein.

In some embodiments, the present disclosure provides a method of treating atypical hemolytic syndrome (aHUS) by administering to a subject in need thereof an effective amount of a compound (e.g., a compound of formula (I), (II), (III), (IIIA), (IV), (IVA), (V), (VA), (VI), (VIA), (VII), or (VILA), or Table 1) or its salt or composition as described herein.

In some embodiments, the present disclosure provides a method of treating age-related macular degeneration (AMD) by administering to a subject in need thereof an effective amount of a compound (e.g., a compound of formula (I), (II), (III), (IIIA), (IV), (IVA), (V), (VA), (VI), (VIA), (VII), or (VILA), or Table 1) or its salt or composition as described herein.

In some embodiments, the present disclosure provides a method of treating rheumatoid arthritis by administering to a subject in need thereof an effective amount of a compound (e.g., a compound of formula (I), (II), (III), (IIIA), (IV), (IVA), (V), (VA), (VI), (VIA), (VII), or (VILA), or Table 1) or its salt or composition as described herein.

In some embodiments, the present disclosure provides a method of treating multiple sclerosis by administering to a subject in need thereof an effective amount of a compound (e.g., a compound of formula (I), (II), (III), (IIA), (IV), (IVA), (V), (VA), (VI), (VIA), (VII), or (VILA), or Table 1) or its salt or composition as described herein.

In some embodiments, the present disclosure provides a method of treating myasthenia gravis by administering to a subject in need thereof an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, the present disclosure provides a method of treating atypical hemolytic uremic syndrome (aHUS) by administering to a subject in need thereof an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In another embodiment, the present disclosure provides a method of treating a disorder as described below by administering to a subject in need thereof an effective amount of a compound (e.g., a compound of formula (I), (II), (III), (IIIA), (IV), (IVA), (V), (VA), (VI), (VIA), (VII), or (VIIA), or Table 1) or its salt or composition as described herein, including: vitritis, sarcoidosis, syphilis, tuberculosis, or Lyme disease; retinal vasculitis, Eales disease, tuberculosis, syphilis, or toxoplasmosis; neuroretinitis, viral retinitis, or acute retinal necrosis; varicella zoster virus, herpes simplex virus, cytomegalovims, Epstein-Barr vims, lichen planus, or Dengue-associated disease (e.g., hemorraghic Dengue Fever); Masquerade syndrome, contact dermatitis, trauma induced inflammation, UVB induced inflammation, eczema, granuloma annulare, or acne.

In an additional embodiment, the disorder is selected from: acute myocardial infarction, aneurysm, cardiopulmonary bypass, dilated cardiomyopathy, complement activation during cardiopulmonary bypass operations, coronary artery disease, restenosis following stent placement, or percutaneous transluminal coronary angioplasty (PTCA); antibody-mediated transplant rejection, anaphylactic shock, anaphylaxis, allogenic transplant, humoral and vascular transplant rejection, graft dysfunction, graft-versus-subject disease, Graves' disease, adverse drug reactions, or chronic graft vasculopathy; allergic bronchopulmonary aspergillosis, allergic neuritis, drug allergy, radiation-induced lung injury, eosinophilic pneumonia, radiographic contest media allergy, bronchiolitis obliterans, or interstitial pneumonia; parkinsonism-dementia complex, sporadic frontotemporal dementia, frontotemporal dementia with Parkinsonism linked to chromosome 17, frontotemporal lobar degeneration, tangle only dementia, cerebral amyloid angiopathy, cerebrovascular disorder, certain forms of frontotemporal dementia, chronic traumatic encephalopathy (CTE), Parkinson's Disease with dementia (PDD), argyrophilic grain dementia, dementia pugilistica, dementia with Lewy Bodies (DLB), or multi-infarct dementia; Creutzfeldt-Jakob disease, Huntington's disease, multifocal motor neuropathy (MMN), prion protein cerebral amyloid angiopathy, polymyositis, postencephalitic parkinsonism, subacute sclerosing panencephalitis, non-Guamanian motor neuron disease with neurofibrillary tangles, neural regeneration, and diffuse neurofibrillary tangles with calcification.

In some embodiments, the disorder is selected from: atopic dermatitis, dermatitis, dermatomyositis bullous pemphigoid, scleroderma, sclerodermatomyositis, psoriatic arthritis, pemphigus vulgaris, Discoid lupus erythematosus, cutaneous lupus, chilblain lupus erythematosus, or lupus erythematosus-lichen planus overlap syndrome; cryoglobulinemic vasculitis, mesenteric/enteric vascular disorder, peripheral vascular disorder, antineutrophil cytoplasm antibody (ANCA)-associated vasculitis (AAV), IL-2 induced vascular leakage syndrome, or immune complex vasculitis;angioedema, low platelets (HELLP) syndrome, sickle cell disease, platelet refractoriness, red cell casts, or typical or infectious hemolytic uremic syndrome (tHUS); hematuria, hemorrhagic shock, drug-induced thrombocytopenia, autoimmune hemolytic anemia (AIHA), azotemia, blood vessel and/or lymph vessel inflammation, rotational atherectomy, or delayed hemolytic transfusion reaction; British type amyloid angiopathy, Buerger's disease, bullous pemphigoid, C1q nephropathy, cancer, and catastrophic antiphospholipid syndrome. In some embodiments, the disorder is autoimmune hemolytic anemia, e.g., warm autoimmune hemolytic anemia.

In another embodiment, the disorder is selected from: wet (exudative) AMD, dry (non-exudative) AMD, chorioretinal degeneration, choroidal neovascularization (CNV), choroiditis, loss of RPE function, loss of vision (including loss of visual acuity or visual field), loss of vision from AMD, retinal damage in response to light exposure, retinal degeneration, retinal detachment, retinal dysfunction, retinal neovascularization (RNV), retinopathy of prematurity, pathological myopia, or RPE degeneration; pseudophakic bullous keratopathy, symptomatic macular degeneration related disorder, optic nerve degeneration, photoreceptor degeneration, cone degeneration, loss of photoreceptor cells, pars planitis, scleritis, proliferative vitreoretinopathy, or formation of ocular drusen; chronic urticaria, Churg-Strauss syndrome, cold agglutinin disease (CAD), corticobasal degeneration (CBD), cryoglobulinemia, cyclitis, damage of the Bruch's membrane, Degos disease, diabetic angiopathy, elevated liver enzymes, endotoxemia, epidermolysis bullosa, or epidermolysis bullosa acquisita; essential mixed cryoglobulinemia, excessive blood urea nitrogen-BUN, focal segmental glomerulosclerosis, Gerstmann-Straussler-Scheinker disease, giant cell arteritis, gout, Hallervorden-Spatz disease, Hashimoto's thyroiditis, Henoch-Schonlein purpura nephritis, or abnormal urinary sediments; hepatitis, hepatitis A, hepatitis B, hepatitis C or human immunodeficiency virus (HIV), a viral infection more generally, for example selected from Flaviviridae, Retroviruses, Coronaviridae, Poxviridae, Adenoviridae, Herpesviridae, Caliciviridae, Reoviridae, Picornaviridae, Togaviridae, Orthomyxoviridae, Rhabdoviridae, or Hepadnaviridae; Neisseria meningitidis, shiga toxin E. coli-related hemolytic uremic syndrome (STEC-HUS), hemolytic uremic syndrome (HUS); Streptococcus, and poststreptococcal glomerulonephritis.

In a further embodiment, the disorder is selected from: hyperlipidemia, hypertension, hypoalbuminemia, hypobolemic shock, hypocomplementemic urticarial vasculitis syndrome, hypophosphastasis, hypovolemic shock, idiopathic pneumonia syndrome, or idiopathic pulmonary fibrosis; inclusion body myositis, intestinal ischemia, iridocyclitis, iritis, juvenile chronic arthritis, Kawasaki's disease (arteritis), or lipiduria; membranoproliferative glomerulonephritis (MPGN) I, microscopic polyangiitis, mixed cryoglobulinemia, molybdenum cofactor deficiency (MoCD) type A, pancreatitis, panniculitis, Pick's disease, polyarteritis nodosa (PAN), progressive subcortical gliosis, proteinuria, reduced glomerular filtration rate (GFR), or renovascular disorder; multiple organ failure, multiple system atrophy (MSA), myotonic dystrophy, Niemann-Pick disease type C, chronic demyelinating diseases, or progressive supranuclear palsy; spinal cord injury, spinal muscular atrophy, spondyloarthropathies, Reiter's syndrome, spontaneous fetal loss, recurrent fetal loss, pre-eclampsia, synucleinopathy, Takayasu's arteritis, post-partum thyroiditis, thyroiditis, Type I cryoglobulinemia, Type II mixed cryoglobulinemia, Type III mixed cryoglobulinemia, ulcerative colitis, uremia, urticaria, venous gas embolus (VGE), or Wegener's granulomatosis; von Hippel-Lindau disease, histoplasmosis of the eye, hard drusen, soft drusen, pigment clumping, and photoreceptor and/or retinal pigmented epithelia (RPE) loss.

In some embodiments, a compound (e.g., a compound of formula (I), (II), (III), (IIIA), (IV), (IVA), (V), (VA), (VI), (VIA), (VII), or (VIIA), or Table 1) or its salt or composition as described herein is useful for treating a disorder selected from autoimmune oophoritis, endometriosis, autoimmune orchitis, Ord's thyroiditis, autoimmune enteropathy, coeliac disease, Hashimoto's encephalopathy, antiphospholipid syndrome (APLS) (Hughes syndrome), aplastic anemia, autoimmune lymphoproliferative syndrome (Canale-Smith syndrome), autoimmune neutropenia, Evans syndrome, pernicious anemia, pure red cell aplasia, thrombocytopenia, adipose dolorosa (Dercum's disease), adult onset Still's disease, ankylosing spondylitis, CREST syndrome, drug-induced lupus, eosinophilic fasciitis (Shulman's syndrome), Felty syndrome, IgG4-related disease, mixed connective tissue disease (MCTD), palindromic rheumatism (Hench-Rosenberg syndrome), Parry-Romberg syndrome, Parsonage-Turner syndrome, relapsing polychondritis (Meyenburg-Altherr-Uehlinger syndrome), retroperitoneal fibrosis, rheumatic fever, Schnitzler syndrome, fibromyalgia, neuromyotonia (Isaac's disease), paraneoplastic degeneration, autoimmune inner ear disease, Meniere's disease, interstitial cystitis, autoimmune pancreatitis, zika virus-related disorders, chikungunya virus-related disorders, subacute bacterial endocarditis (SBE), IgA nephropathy, IgA vasculitis, polymyalgia rheumatic, rheumatoid vasculitis, alopecia areata, autoimmune progesterone dermatitis, dermatitis herpetiformis, erythema nodosum, gestational pemphigoid, hidradenitis suppurativa, lichen sclerosus, linear IgA disease (LAD), morphea, myositis, pityriasis lichenoides et varioliformis acuta, vitiligo post-myocardial infarction syndrome (Dressler's syndrome), post-pericardiotomy syndrome, autoimmune retinopathy, Cogan syndrome, Graves opthalmopathy, ligneous conjunctivitis, Mooren's ulcer, opsoclonus myoclonus syndrome, optic neuritis, retinocochleocerebral vasculopathy (Susac's syndrome), sympathetic opthalmia, Tolosa-Hunt syndrome, interstitial lung disease, antisynthetase syndrome, Addison's disease, autoimmune polyendocrine syndrome (APS) type I, autoimmune polyendocrine syndrome (APS) type II, autoimmune polyendocrine syndrome (APS) type III, disseminated sclerosis (multiple sclerosis, pattern II), rapidly progressing glomerulonephritis (RPGN), juvenile rheumatoid arthritis, enthesitis-related arthritis, reactive arthritis (Reiter's syndrome), autoimmune hepatitis or lupoid hepatitis, primary biliary cirrhosis (PBS), primary sclerosing cholangitis, microscopic colitis, latent lupus (undifferentiated connective tissue disease (UCTD)), acute disseminated encephalomyelitis (ADEM), acute motor axonal neuropathy, anti-n-methyl-D-aspartate receptor encephalitis, Balo concentric sclerosis (Schilders disease), Bickerstaff's encephalitis, chronic inflammatory demyelinating polyneuropathy, idiopathic inflammatory demyelinating disease, Lambert-Eaton mysathenic syndrome, Oshtoran syndrome, pediatric autoimmune neuropsychiatric disorder associated with streptococcus (PANDAS), progressive inflammatory neuropathy, restless leg syndrome, stiff person syndrome, Sydenhem syndrome, transverse myelitis, lupus vasculitis, leukocytoclastic vasculitis, Microscopic Polyangiitis, polymyositis, and ischemic-reperfusion injury of the eye.

Examples of eye disorders that may be treated according to the compositions and methods disclosed herein include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma, and combinations thereof.

In a further embodiment, the disorder is selected from glaucoma, diabetic retinopathy, blistering cutaneous diseases (including bullous pemphigoid, pemphigus, and epidermolysis bullosa), ocular cicatrical pemphigoid, uveitis, adult macular degeneration, diabetic retinopa retinitis pigmentosa, macular edema, diabetic macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonatretic ischemic optic neuropathy, postoperative inflammation, and retinal vein occlusion, and central retinal vein occlusion (CVRO).

In some embodiments, a method for the treatment of an autoimmune blistering disease in a subject is provided that includes the administration of an effective amount of a compound (e.g., a compound of formula (I), (II), (III), (IIIA). (IV), (IVA), (V), (VA), (VI), (VIA), (VII), or (VIIA), or Table 1) or its salt or composition as described herein.

In some embodiments, a method for the treatment of bullous pemphigoid in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, the complement mediated disorder is an ophthalmic disease (e.g., early or neovascular age-related macular degeneration and geographic atrophy), an autoimmune disease (e.g., arthritis or rheumatoid arthritis), a respiratory diseases, or a cardiovascular disease. In other embodiments, the compounds of the disclosure are suitable for use in the treatment of diseases and disorders associated with fatty acid metabolism, including obesity and other metabolic disorders.

In some embodiments, a method for the treatment of geographic atrophy in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

Disorders that may be treated or prevented by a compound (e.g., a compound of formula (I), (II), (III), (TITA), (IV), (IVA), (V), (VA), (VI), (VIA), (VII), or (VIIA), or Table 1) or its salt or composition as described herein also include, but are not limited to: hereditary angioedema, capillary leak syndrome, hemolytic uremic syndrome (HUS), neurological disorders, Guillain-Barre Syndrome, diseases of the central nervous system and other neurodegenerative conditions, glomemlonephritis (including membrane proliferative glomerulonephritis), SLE nephritis, proliferative nephritis, liver fibrosis, tissue regeneration and neural regeneration, or Barraquer-Simons Syndrome; inflammatory effects of sepsis, systemic inflammatory response syndrome (SIRS), disorders of inappropriate or undesirable complement activation, interleukin-2 induced toxicity during IL-2 therapy, inflammatory disorders, inflammation of autoimmune diseases, systemic lupus erythematosus (SLE), lupus nephritis, arthritis, immune complex disorders and autoimmune diseases, systemic lupus, or lupus erythematosus; ischemia/reperfusion injury (I/R injury), myocardial infarction, myocarditis, post-ischemic reperfusion conditions, balloon angioplasty, atherosclerosis, post-pump syndrome in cardiopulmonary bypass or renal bypass, renal ischemia, mesenteric artery reperfusion after aortic reconstruction, antiphospholipid syndrome, autoimmune heart disease, ischemia-reperfusion injuries, obesity, or diabetes; Alzheimer's dementia, stroke, schizophrenia, traumatic brain injury, trauma, Parkinson's disease, epilepsy, transplant rejection, prevention of fetal loss, biomaterial reactions (e.g. in hemodialysis, implants), hyperacute allograft rejection, xenograft rejection, transplantation, psoriasis, burn injury, thermal injury including burns or frostbite, or crush injury; asthma, allergy, acute respiratory distress syndrome (ARDS), cystic fibrosis, adult respiratory distress syndrome, dyspnea, hemoptysis, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), bronchoconstriction, hypersensitivity pneumonitis, parasitic diseases, Goodpasture's Syndrome (anti-glomerular basement membrane nephritis), pulmonary vasculitis, Pauci-immune vasculitis, and immune complex-associated inflammation.

In some embodiments, a method for the treatment of sickle cell disease in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, a method for the treatment of immune thrombocytopenic purpura (ITP), thrombotic thrombocytopenic purpura (TTP), or idiopathic thrombocytopenic purpura (ITP) in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein. In some embodiments, the disorder to be treated is ITP.

In some embodiments, a method for the treatment of ANCA-vasculitis in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, a method for the treatment of IgA nephropathy in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, a method for the treatment of rapidly progressing glomerulonephritis (RPGN), in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, a method for the treatment of lupus nephritis, in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In some embodiments, a method for the treatment of hemorraghic dengue fever, in a subject is provided that includes the administration of an effective amount of a compound (e.g., a compound of formula (I), (II), (III), (IIIA), (IV), (IVA), (V), (VA), (VI), (VIA), (VII), or (VIIA), or Table 1) or its salt or composition as described herein.

In an additional alternative embodiment, a compound (e.g., a compound of formula (I), (II), (III), (IIIA), (IV), (IVA), (V), (VA), (VI), (VIA), (VII), or (VIIA), or Table 1) or its salt or composition as described herein is used in the treatment of an autoimmune disorder. The complement pathway enhances the ability of antibodies and phagocytic cells to clear microbes and damaged cells from the body. It is part of the innate immune system and in healthy individuals is an essential process. Inhibiting the complement pathway will decrease the body's immune system response. Therefore, it is an object of the present disclosure to treat autoimmune disorders by administering an effective does of a compound (e.g., a compound of formula (I), (II), (III), (IIIA), (IV), (IVA), (V), (VA), (VI), (VIA), (VII), or (VIIA), or Table 1) or its salt or composition as described herein to a subject in need thereof.

In some embodiments, the autoimmune disorder is caused by activity of the complement system. In some embodiments the autoimmune disorder is caused by activity of the alternative complement pathway. In some embodiments the autoimmune disorder is caused by activity of the classical complement pathway. In another embodiment the autoimmune disorder is caused by a mechanism of action that is not directly related to the complement system, such as the over-proliferation of T-lymphocytes or the over-production of cytokines.

Non-limiting examples of autoimmune disorders include: lupus, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), diabetes, multiple sclerosis, pernicious anemia, psoriasis, rheumatoid arthritis, sarcoidosis, and scleroderma.

In some embodiments, a compound (e.g., a compound of formula (I), (II), (III), (IIIA), (IV), (IVA), (V), (VA), (VI), (VIA), (VII), or (VIIA), or Table 1) or its salt or composition as described herein is used in the treatment of lupus. Non-limiting examples of lupus include lupus erythematosus, cutaneous lupus, discoid lupus erythematosus, chilblain lupus erythematosus, and lupus erythematosus-lichen planus overlap syndrome.

Lupus erythematosus is a general category of disease that includes both systemic and cutaneous disorders. The systemic form of the disease can have cutaneous as well as systemic manifestations. However, there are also forms of the disease that are only cutaneous without systemic involvement. For example, SLE is an inflammatory disorder of unknown etiology that occurs predominantly in women, and is characterized by articular symptoms, butterfly erythema, recurrent pleurisy, pericarditis, generalized adenopathy, splenomegaly, as well as CNS involvement and progressive renal failure. The sera of most patients (over 98%) contain antinuclear antibodies, including anti-DNA antibodies. High titers of anti-DNA antibodies are essentially specific for SLE. Conventional treatment for this disease has been the administration of corticosteroids or immunosuppressants.

There are three forms of cutaneous lupus: chronic cutaneous lupus (also known as discoid lupus erythematosus or DLE), subacute cutaneous lupus, and acute cutaneous lupus. DLE is a disfiguring chronic disorder primarily affecting the skin with sharply circumscribed macules and plaques that display erythema, follicular plugging, scales, telangiectasia, and atrophy. The condition is often precipitated by sun exposure, and the early lesions are erythematous, round scaling papules that are 5 to 10 mm in diameter and display follicular plugging. DLE lesions appear most commonly on the cheeks, nose, scalp, and ears, but they may also be generalized over the upper portion of the trunk, extensor surfaces of the extremities, and on the mucous membranes of the mouth. If left untreated, the central lesion atrophies and leaves a scar. Unlike SLE, antibodies against double-stranded DNA (e.g., DNA-binding test) are almost invariably absent in DLE.

Diabetes can refer to either type 1 or type 2 diabetes. In some embodiments a compound (e.g., a compound of formula (I), (II), (III), (IIIA), (IV), (IVA), (V), (VA), (VI), (VIA), (VII), or (VIIA), or Table 1) or its salt or composition as described herein is provided at an effective dose to treat a patient with type 1 diabetes. In some embodiments a compound (e.g., a compound of formula (I), (IL), (III), (IIIA), (IV), (IVA), (V), (VA), (VI), (VIA), (VII), or (VIIA), or Table 1) or its salt or composition as described herein is provided at an effective dose to treat a patient with type 2 diabetes.

Type 1 diabetes is an autoimmune disease. An autoimmune disease results when the body's system for fighting infection (the immune system) attacks a part of the body. In the case of diabetes type 1, the pancreas then produces little or no insulin.

In some embodiments, the complement-mediated disease or disorder comprises transplant rejection. In some embodiments, the complement-mediated disease or disorder is antibody-mediated transplant rejection.

In certain aspects, a compound (e.g., a compound of formula (I), (II), (III), (IIIA), (IV), (IVA), (V), (VA), (VI), (VIA), (VII), or (VIIA), or Table 1) or its salt or composition as described herein is used to treat a proliferative disorder, including, but not limited to, cancer. Targeted cancers suitable for administration of a compound (e.g., a compound of formula (I), (II), (III), (IIIA), (IV), (IVA), (V), (VA), (VI), (VIA), (VII), or (VIIA), or Table 1) or its salt described herein include, but are not limited to, estrogen-receptor positive cancer, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, adenocarcinoma of the colon, adenocarcinoma of the rectum, central nervous system germ cell tumors, teratomas, estrogen receptor-negative breast cancer, estrogen receptor-positive breast cancer, familial testicular germ cell tumors, HER2-negative breast cancer, HER2-positive breast cancer, male breast cancer, ovarian immature teratomas, ovarian mature teratoma, ovarian monodermal and highly specialized teratomas, progesterone receptor-negative breast cancer, progesterone receptor-positive breast cancer, recurrent breast cancer, recurrent colon cancer, recurrent extragonadal germ cell tumors, recurrent extragonadal non-seminomatous germ cell tumor, recurrent extragonadal seminomas, recurrent malignant testicular germ cell tumors, recurrent melanomas, recurrent ovarian germ cell tumors, recurrent rectal cancer, stage III extragonadal non-seminomatous germ cell tumors, stage III extragonadal seminomas, stage III malignant testicular germ cell tumors, stage III ovarian germ cell tumors, stage IV breast cancers, stage IV colon cancers, stage IV extragonadal non-seminomatous germ cell tumors, stage IV extragonadal seminoma, stage IV melanomas, stage IV ovarian germ cell tumors, stage IV rectal cancers, testicular immature teratomas, testicular mature teratomas. In particular embodiments, the targeted cancers included estrogen-receptor positive, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, metastatic colorectal cancer, metastatic melanoma with CDK4 mutation or amplification, or cisplatin-refractory, unresectable germ cell tumors, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, fibrosarcoma, myxosarcoma, chondrosarcoma, osteosarcoma, chordoma, malignant fibrous histiocytoma, hemangiosarcoma, angiosarcoma, lymphangiosarcoma, Mesothelioma, leiomyosarcoma, rhabdomyosarcoma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phyllode, salivary cancers, thymic carcinomas, bladder cancer, and Wilms tumor, a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others, T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézaiy syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma, primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hepatosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In some embodiments, the methods described herein can be used to treat a subject, for example a human, with a lymphoma or lymphocytic or myelocytic proliferation disorder or abnormality. For example, the methods as described herein can be administered to a subject with a Hodgkin Lymphoma or a Non-Hodgkin Lymphoma. For example, the subject can have a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma: Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia, a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL, a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia: Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+DLBCL of the elderly; Primary mediastinal (thymic) large B-cell lymphoma: Primary cutaneous DLBCL, leg type; ALK+large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma, a leukemia, for example, an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AMIL); large granular lymphocytic leukemia; or Adult T-cell chronic leukemia. In some embodiments, the patient has an acute myelogenous leukemia, for example an undifferentiated AML (M0); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia[M4E]); monocytic leukemia (M5); erythroleukemia (M6); or megakaryoblastic leukemia (M7), small cell lung cancer, retinoblastoma, HPV positive malignancies like cervical cancer and certain head and neck cancers, MYC amplified tumors such as Burkitts' Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancers, and certain classes of testicular cancers.

In certain aspects, a compound (e.g., a compound of formula (I), (II), (III), (IIIA), (IV), (IVA), (V), (VA), (VI), (VIA), (VII), or (VIIA), or Table 1) or its salt as described herein can be used to preserve or prevent damage to an organ or blood product. For example, a compound (e.g., a compound of formula (I), (II), (III), (IIIA), (IV), (IVA), (V), (VA), (VI), (VIA), (VII), or (VIIA), or Table 1) or its salt described herein can be used to prevent damage to an organ, tissue, cell product, or blood product, that has been harvested for transplantation. In some embodiments, the organ is the heart, kidney, pancreas, lung, liver, or intestine. In some embodiments, the tissue is derived from the cornea, bone, tendon, muscle, heart valve, nerve, artery or vein, or the skin. In some embodiments, the blood product is whole blood, plasma, red blood cells or reticulocytes.

In some embodiments, a compound (e.g., a compound of formula (I), (II), (III), (IIIA), (IV), (IVA), (V), (VA), (VI), (VIA), (VII), or (VIIA), or Table 1) or its salt or composition as described herein prevents or delays the onset of at least one symptom of a complement-mediated disease or disorder in an individual. In some embodiments, a compound (e.g., a compound of formula (I), (II), (III), (IIIA), (IV), (IVA), (V), (VA), (VI), (VIA), (VII), or (VIIA), or Table 1) or its salt or composition as described herein reduces or eliminates at least one symptom of a complement-mediated disease or disorder in an individual. Examples of symptoms include, but are not limited to, symptoms associated with autoimmune disease, cancer, hematological disease, infectious disease, inflammatory disease, ischemia-reperfusion injury, neurodegenerative disease, neurodegenerative disorder, renal disease, transplant rejection, ocular disease, vascular disease, or a vasculitis disorder. The symptom can be a neurological symptom, for example, impaired cognitive function, memory impairment, loss of motor function, etc. The symptom can also be the activity of Cis protein in a cell, tissue, or fluid of an individual. The symptom can also be the extent of complement activation in a cell, tissue, or fluid of an individual.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein to an individual modulates complement activation in a cell, tissue, or fluid of an individual. In some embodiments, administration of a compound (e.g., a compound of formula (I), (II), (III), (IIIA), (IV), (IVA), (V), (VA), (VI), (VIA), (VII), or (VIIA), or Table 1) or its salt or composition as described herein to an individual inhibits complement activation in a cell, tissue, or fluid of an individual. For example, in some embodiments, a compound (e.g., a compound of formula (I), (II), (III), (IIIA), (IV), (IVA), (V), (VA), (VI), (VIA), (VII), or (VIIA), or Table 1) or its salt or composition as described herein, when administered in one or more doses as monotherapy or in combination therapy to an individual having a complement-mediated disease or disorder, inhibits complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the compounds described herein.

In some embodiments, a compound (e.g., a compound of formula (I), (II), (III), (IIIA), (IV), (IVA), (V), (VA), (VI), (VIA), (VII), or (VIIA), or Table 1) or its salt or composition as described herein reduces C3 deposition onto red blood cells; for example, in some embodiments, a compound (e.g., a compound of formula (I), (II), (III), (IIIA), (IV), (IVA), (V), (VA), (VI), (VIA), (VII), or (VIIA), or Table 1) or its salt or composition as described herein reduces deposition of C3b, iC3b, etc., onto RBCs. In some embodiments, a compound (e.g., a compound of formula (I), (II), (III), (IIIA), (IV), (IVA), (V), (VA), (VI), (VIA), (VII), or (VIIA), or Table 1) or its salt or composition as described herein inhibits complement-mediated red blood cell lysis.

In some embodiments, a compound (e.g., a compound of formula (I), (II), (III), (IIIA), (IV), (IVA), (V), (VA), (VI), (VIA), (VII), or (VIIA), or Table 1) or its salt or composition as described herein reduces C3 deposition onto platelets; for example, in some embodiments, a compound (e.g., a compound of formula (I), (II), (III), (IIIA), (IV), (IVA), (V), (VA), (VI), (VIA), (VII), or (VIIA), or Table 1) or its salt or composition as described herein reduces deposition of C3b, iC3b, etc., onto platelets.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein results in an outcome selected from the group consisting of: (a) a reduction in complement activation; (b) an improvement in cognitive function; (c) a reduction in neuron loss; (d) a reduction in phospho-Tau levels in neurons; (e) a reduction in glial cell activation; (f) a reduction in lymphocyte infiltration; (g) a reduction in macrophage infiltration; (h) a reduction in antibody deposition, (i) a reduction in glial cell loss; (j) a reduction in oligodendrocyte loss; (k) a reduction in dendritic cell infiltration; (1) a reduction in neutrophil infiltration; (m) a reduction in red blood cell lysis; (n) a reduction in red blood cell phagocytosis; (o) a reduction in platelet phagocytosis; (p) a reduction in platelet lysis; (q) an improvement in transplant graft survival; (r) a reduction in macrophage mediated phagocytosis; (s) an improvement in vision; (t) an improvement in motor control; (u) an improvement in thrombus formation; (v) an improvement in clotting; (w) an improvement in kidney function; (x) a reduction in antibody mediated complement activation; (y) a reduction in autoantibody mediated complement activation; (z) an improvement in anemia; (aa) reduction of demyelination; (ab) reduction of eosinophilia; (ac) a reduction of C3 deposition on red blood cells (e.g., a reduction of deposition of C3b, iC3b, etc., onto RBCs); and (ad) a reduction in C3 deposition on platelets (e.g., a reduction of deposition of C3b, iC3b, etc., onto platelets); and (ae) a reduction of anaphylatoxin toxin production; (af) a reduction in autoantibody mediated blister formation; (ag) a reduction in autoantibody induced pruritis; (ah) a reduction in autoantibody induced erythematosus; (ai) a reduction in autoantibody mediated skin erosion; (aj) a reduction in red blood cell destruction due to transfusion reactions; (ak) a reduction in red blood cell lysis due to alloantibodies; (al) a reduction in hemolysis due to transfusion reactions; (am) a reduction in allo-antibody mediated platelet lysis; (an) a reduction in platelet lysis due to transfusion reactions; (ao) a reduction in mast cell activation; (ap) a reduction in mast cell histamine release; (aq) a reduction in vascular permeability; (ar) a reduction in edema; (as) a reduction in complement deposition on transplant graft endothelium; (at) a reduction of anaphylatoxin generation in transplant graft endothelium; (au) a reduction in the separation of the dermal-epidermal junction; (av) a reduction in the generation of anaphylatoxins in the dermal-epidermal junction; (aw) a reduction in alloantibody mediated complement activation in transplant graft endothelium; (ax) a reduction in antibody mediated loss of the neuromuscular junction; (ay) a reduction in complement activation at the neuromuscular junction; (az) a reduction in anaphylatoxin generation at the neuromuscular junction; (ba) a reduction in complement deposition at the neuromuscular junction; (bb) a reduction in paralysis: (bc) a reduction in numbness; (bd) increased bladder control; (be) increased bowel control; (bf) a reduction in mortality associated with autoantibodies; and (bg) a reduction in morbidity associated with autoantibodies.

In some embodiments, a compound (e.g., a compound of formula (I), (II), (III), (IIIA), (IV), (IVA), (V), (VA), (VI), (VIA), (VII), or (VIIA), or Table 1) or its salt or composition as described herein, when administered in one or more doses to an individual having a complement-mediated disease or disorder, is effective to achieve a reduction of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: (a) complement activation; (b) decline in cognitive function; (c) neuron loss; (d) phospho-Tau levels in neurons; (e) glial cell activation; (f) lymphocyte infiltration; (g) macrophage infiltration; (h) antibody deposition, (i) glial cell loss; ( ) oligodendrocyte loss; (k) dendritic cell infiltration; (1) neutrophil infiltration; (m) red blood cell lysis; (n) red blood cell phagocytosis; (o) platelet phagocytosis; (p) platelet lysis; (q) transplant graft rejection; (r) macrophage mediated phagocytosis; (s) vision loss; (t) antibody mediated complement activation; (u) autoantibody mediated complement activation; (v) demyelination; (w) eosinophilia; compared to the level or degree of the outcome in the individual before treatment with the active compound or its salt.

In some embodiments, a compound (e.g., a compound of formula (I), (II), (III), (IIIA), (IV), (IVA), (V), (VA), (VI), (VIA), (VII), or (VIIA), or Table 1) or its salt or composition as described herein, when administered in one or more doses to an individual having a complement-mediated disease or disorder, is effective to achieve an improvement of at least about 10%, at least about 15%, at least about 20%, at least about 25%, at least about 30%, at least about 40%, at least about 50%, at least about 60%, at least about 70%, at least about 80%, at least about 90%, or more than 90%, of one or more of the following outcomes: a) cognitive function; b) transplant graft survival; c) vision; d) motor control; e) thrombus formation; f) clotting; g) kidney function; and h) hematocrit (red blood cell count), compared to the level or degree of the outcome in the individual before treatment with the active compound or its salt.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein to an individual reduces complement activation in the individual. For example, in some embodiments, a compound (e.g., a compound of formula (I), (II), (III), (IIIA), (IV), (IVA), (V), (VA), (VI), (VIA), (VII), or (VITA), or Table 1) or its salt or composition as described herein, when administered in one or more doses to an individual having a complement-mediated disease or disorder, reduces complement activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to complement activation in the individual before treatment with the active compound or its salt.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein improves cognitive function in the individual. For example, in some embodiments, a compound (e.g., a compound of formula (I), (II), (III), (IIIA), (IV), (IVA), (V), (VA), (VI), (VIA), (VII), or (VIIA), or Table 1) described herein, when administered in one or more doses to an individual having a complement-mediated disease or disorder, improves cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the cognitive function in the individual before treatment with the active compound or its salt.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein reduces the rate of decline in cognitive function in the individual. For example, in some embodiments, a compound (e.g., a compound of formula (I), (II), (III), (IIIA), (IV), (IVA), (V), (VA), (VI), (VIA), (VII), or (VIIA), or Table 1) or its salt, when administered in one or more doses to an individual having a complement-mediated disease or disorder, reduces the rate of decline of cognitive function in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the rate of decline in cognitive function in the individual before treatment with the active compound or its salt.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein to an individual reduces neuron loss in the individual. For example, in some embodiments, a compound (e.g., a compound of formula (I), (II), (III), (IIIA), (IV), (IVA), (V), (VA), (VI), (VIA), (VII), or (VIIA), or Table 1) or its salt, when administered in one or more doses to an individual having a complement-mediated disease or disorder, reduces neuron loss in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to neuron loss in the individual before treatment with the active compound or its salt.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein to an individual reduces phospho-Tau levels in the individual. For example, in some embodiments, a compound (e.g., a compound of formula (I), (II), (III), (IIIA), (IV), (IVA), (V), (VA), (VI), (VIA), (VII), or (VIIA), or Table 1) or its salt, when administered in one or more doses to an individual having a complement-mediated disease or disorder, reduces phospho-Tau in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the phospho-Tau level in the individual before treatment with the active compound or its salt.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein to an individual reduces glial cell activation in the individual. For example, in some embodiments, a compound (e.g., a compound of formula (I), (II), (III), (IIIA), (IV), (IVA), (V), (VA), (VI), (VIA), (VII), or (VIIA), or Table 1) or its salt, when administered in one or more doses to an individual having a complement-mediated disease or disorder, reduces glial activation in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to glial cell activation in the individual before treatment with the active compound or its salt. In some embodiments, the glial cells are astrocytes or microglia In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein to an individual reduces lymphocyte infiltration in the individual. For example, in some embodiments, a compound (e.g., a compound of formula (I), (II), (III), (IIIA), (IV), (IVA), (V), (VA), (VI), (VIA), (VII), or (VIIA), or Table 1) or its salt, when administered in one or more doses to an individual having a complement-mediated disease or disorder, reduces lymphocyte infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to lymphocyte infiltration in the individual before treatment with the active compound or its salt.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein to an individual reduces macrophage infiltration in the individual. For example, in some embodiments, a compound (e.g., a compound of formula (I), (II), (III), (IIIA), (IV), (IVA), (V), (VA), (VI), (VIA), (VII), or (VIIA), or Table 1) or its salt, when administered in one or more doses to an individual having a complement-mediated disease or disorder, reduces macrophage infiltration in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to macrophage infiltration in the individual before treatment with the active compound or its salt.

In some embodiments, administering a compound (e.g., a compound of formula (I), (II), (III), (IIIA), (IV), (IVA), (V), (VA), (VI), (VIA), (VII), or (VITA), or Table 1) or its salt or composition as described herein to an individual reduces antibody deposition in the individual. For example, in some embodiments, a compound (e.g., a compound of formula (I), (II), (III), (IIIA), (IV), (IVA), (V), (VA), (VI), (VIA), (VII), or (VIIA), or Table 1) or its salt, when administered in one or more doses to an individual having a complement-mediated disease or disorder, reduces antibody deposition in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to antibody deposition in the individual before treatment with the active compound or its salt.

In some embodiments, administering a compound (e.g., a compound of formula (I), (II), (III), (IIIA), (IV), (IVA), (V), (VA), (VI), (VIA), (VII), or (VIIA), or Table 1) or its salt or composition as described herein to an individual reduces anaphylatoxin (e.g., C₃a, C₄a, C₅a) production in an individual. For example, in some embodiments, a compound (e.g., a compound of formula (I), (II), (III), (IIIA), (IV), (IVA), (V), (VA), (VI), (VIA), (VII), or (VIIA), or Table 1) or its salt, when administered in one or more doses to an individual having a complement-mediated disease or disorder, reduces anaphylatoxin production in the individual by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of anaphylatoxin production in the individual before treatment with the active compound or its salt.

The present disclosure provides a use of a compound (e.g., a compound of formula (I), (II), (III), (IIIA), (IV), (IVA), (V), (VA), (VI), (VIA), (VII), or (VIIA), or Table 1) or its salt of the present disclosure or a pharmaceutical composition comprising a compound (e.g., a compound of formula (I), (II), (III), (IIIA), (IV), (IVA), (V), (VA), (VI), (VIA), (VII), or (VIIA), or Table 1) or its salt of the present disclosure and a pharmaceutically acceptable excipient to treat an individual having a complement-mediated disease or disorder. In some embodiments, the present disclosure provides a use of a compound (e.g., a compound of formula (I), (II), (III), (IIIA), (IV), (IVA), (V), (VA), (VI), (VIA), (VII), or (VIIA), or Table 1) or its salt of the present disclosure to treat an individual having a complement-mediated disease or disorder. In some embodiments, the present disclosure provides a use of a pharmaceutical composition comprising a compound (e.g., a compound of formula (I), (II), (III), (IIIA), (IV), (IVA), (V), (VA), (VI), (VIA), (VII), or (VIIA), or Table 1) or its salt of the present disclosure and a pharmaceutically acceptable excipient to treat an individual having a complement-mediated disease or disorder.

### EXAMPLES

The following examples are merely illustrative and should not be construed as limiting the scope of this disclosure in any way as many variations and equivalents will become apparent to those skilled in the art upon reading the present disclosure. The contents of all references, patents, and patent applications cited throughout this application are expressly incorporated herein by reference.

### Example 1. Non-Limiting Synthetic Examples of Compounds of the Present Disclosure

The below schemes are non-limiting examples of methods to make compounds of the present disclosure. The skilled artisan will recognize that there are various modifications that can be performed to make analogs or prepare compounds in other ways.

### ABBREVATIONS

| | |
|---|---|
| Ac₂O | acetic anhydride |
| AcOH | acetic acid |
| BH₃•Me₂S | borane dimethylsulfide |
| BF₃•Et₂O | boron trifluoride etherate |
| Boc₂O | di-tert-butyl dicarbonate |
| BnBr | benzyl bromide |
| MeOH | methanol |
| DBU | 1,8-diazabicyclo[5.4.0]undec-7-ene |
| DCM | dichloromethane |
| DIBAL-H | diisobutylaluminium hydride |
| DIEA, | N,N-diisopropylethylamine |
| DIPEA | |
| DMAP | 4-dimethylaminopyridine |
| DMF | N,N-dimethylformamide |
| DMSO | dimethylsulfoxide |
| DPPA | diphenyl phosphoryl azide |
| EtOAc | ethyl acetate |
| EtOH | ethanol |
| Et₃N or TEA | trimethylamine |
| FA | formic acid |
| HATU | 1-[bis(dimethylamino)methylene]-1H-1,2,3-triazolo[4,5- |
| | b]pyridinium3- oxide hexafluorophosphate |
| HBTU | (2-(1H-benzotriazol-1-yl)-1,1,3,3-tetramethyluronium |
| | hexafluorophosphate |
| LDA | lithium diisopropylamide |
| MeCN | acetonitrile |
| MeI | methyl iodide |
| MsCl | mesylchloride |
| NBS | N-bromo succinimide |
| NMP | N-methyl-2-pyrrolidone |
| Pd(dppf)Cl₂ | [1,1′-bis(diphenylphosphino) ferrocene]dichloro- |
| | palladium(II) |
| Pd(PPh₃)₄ | tetrakis(triphenylphosphine)palladium(0) |
| Pd(PPh₃)₂Cl₂ | bis(triphenylphosphine)palladium(II) dichloride |
| Pd/C | palladium on carbon |
| PE | petroleum ether |
| PhSO₂Cl | benzenesulfonyl chloride |
| PPh₃ | triphenylphosphine |
| PyBOP | benzotriazol-1-yl-oxytripyrrolidinophosphonium |
| | hexafluorophosphate |
| T3P or T₃P | propane phosphonic acid anhydride |
| t-BuOK | potassium tert-butoxide |
| Tf₂O | trifluoromethanesulfonic anhydride |
| TFA | trifluoroacetic acid |
| THF | tetrahydrofuran |
| Ti(OEt)₄ | titanium (IV) ethoxide |
| TMEDA | N,N,N′,N′-tetramethylethylenediamine |
| TMSCHN₂ | trimethylsilyldiazomethane |

### General Methods

All nonaqueous reactions were performed under an atmosphere of dry argon or nitrogen gas using anhydrous solvents. The progress of reactions and the purity of target compounds were determined using one of the two liquid chromatography (LC) methods A or B disclosed herein. The structure of starting materials, intermediates, and final products was confirmed by standard analytical techniques, including NMR spectroscopy and mass spectrometry.

### LC Method A

Instrument: Waters Acquity Ultra Performance LC
Column: ACQUITY UPLC BEH C18 2.1′ 50 mm, 1.7 mm
Column Temperature: 40° C.
Mobile Phase: Solvent A: H₂O+0.05% FA; Solvent B: CH₃CN+0.05% FA
Flow Rate: 0.8 mL/min
Gradient: 0.24 min @15% B, 3.5 min gradient (15-85% B), then 0.5 min a 85% B.
Detection: UV (210-410 ma) and MS (SQ in ES+ mode)

### Scheme 1. Synthesis of(1S,3S,5S)-N-((7-amino-4,5-dihydrothieno[2,3-c]pyridin-2-yl)methyl)-5-methyl-2-((4-phenoxybenzoyl)glycyl)-2-azabicyclo[3.1.0]hexane-3-carboxamide (Compound 1)

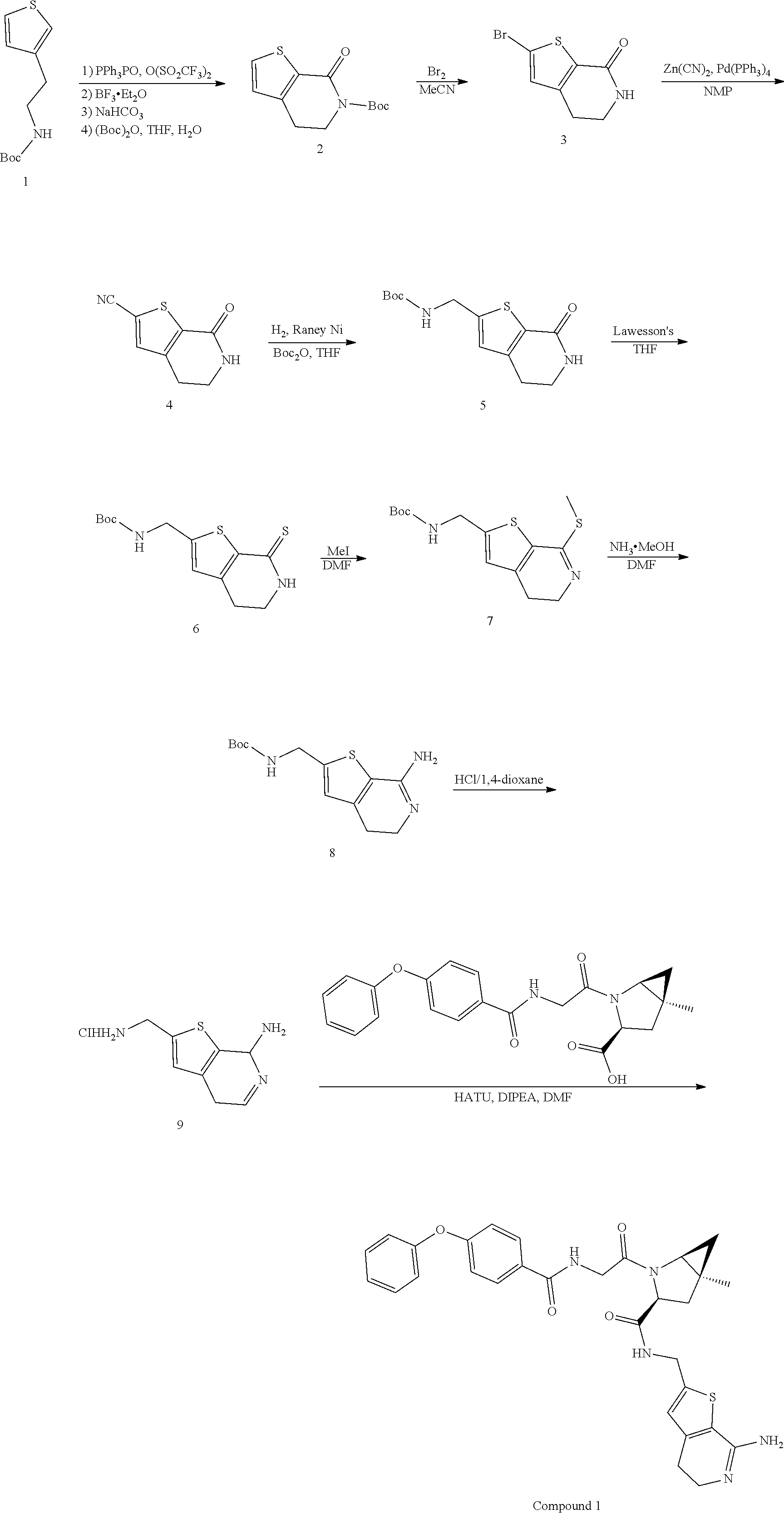

### Step 1: tert-butyl 7-oxo-4,7-dihydrothieno[2,3-c]pyridine-6(5H)-carboxylate

To a solution of PPh₃ (1.91 g, 6.86 mmol) in DCM (10 mL) was added Tf₂O (970 mg, 3.43 mmol) dropwise at −5° C. and stirred at −5° C. for 0.5 hours under N₂ atmosphere. A solution of tert-butyl (2-(thiophen-3-yl)ethyl)carbamate (650 mg, 2.86 mmol) in DCM (10 mL) was added to the stirring reaction mixture and stirred at −5° C. for 15 minutes, then BF₃·Et₂O (2.03 g, 14.30 mmol) was added and stirred at −5° C. for 1 hours. The mixture was quenched with saturated aq. NaHCO₃ solution and diluted with DCM and washed with brine, dried over anhydrous Na₂SO₄, filtered, and concentrated to dryness. The residue was dissolved in THF (10 mL) was added (Boc)₂O (1.87 g, 8.58 nmol) and DMAP (105 mg, 0.86 mmol), the mixture was stirred at room temperature overnight. The mixture was diluted with water and extracted with EtOAc twice. The combined organic layers were washed with brine, dried with anhydrous Na₂SO₄, filtered, and concentrated to dryness. The residue was purified by flash chromatography (0-12% EtOAc in PE) to give tert-butyl 7-oxo-4,7-dihydrothieno[2,3-c]pyridine-6(5H)-carboxylate (600 mg, yield 82.8%) as a white solid. LC/MS (ESI) (m/z): 254 (M+H)⁺.

### Step 2: 2-bromo-5,6-dihydrothieno[2,3-c]pyridin-7(4H)-one

To a solution often-butyl 7-oxo-4,7-dihydrothieno[2,3-c]pyridine-6(5H)-carboxylate (600 mg, 2.37 nmol) in MeCN (6 mL) was added a solution of Br₂(568 mg, 3.55 mmol) in MeCN (2 mL) dropwise and the mixture was stirred at 0° C. for 8 hours. The mixture was quenched with aq. Na₂S₂O₃ solution and extracted with EtOAc twice. The combined organic layers were washed with brine, dried with anhydrous Na₂SO₄, filtered, and concentrated to dryness. The residue was purified by flash chromatography (0-50% EtOAc in PE) to give 2-bromo-5,6-dihydrothieno[2,3-c]pyridin-7(4H)-one (300 mg, yield 54.6%) as a white solid. LC/MS (ESI) (m/z): 232/234 (M+H)⁺.

### Step 3: 7-oxo-4,5,6,7-tetrahydrothieno[2,3-c]pyridine-2-carbonitrile

To a solution of 2-bromo-5,6-dihydrothieno[2,3-c]pyridin-7(4H)-one (100 mg, 0.43 mmol) in NMP (3 mL) was added Zn(CN)₂ (127 mg, 1.08 mmol) and Pd(PPh₃)₄ at 0° C., and the mixture was stirred at 120° C. for 6 hours. The mixture was diluted with water and extracted with EtOAc twice. The combined organic layers were washed with brine, dried with anhydrous Na₂SO₄, filtered, and concentrated to dryness. The residue was purified by flash chromatography (0-60% EtOAc in PE) to give 7-oxo-4,5,6,7-tetrahydrothieno[2,3-c]pyridine-2-carbonitrile (65 mg, yield 84.7%) as a light oil. LC/MS (ESI) m/z: 179 (M+H)⁺.

### Step 4: tert-butyl ((7-oxo-4,5,6,7-tetrahydrothieno[2,3-c]pyridin-2-yl)methyl) carbamate

To a solution of 7-oxo-4,5,6,7-tetrahydrothieno[2,3-c]pyridine-2-carbonitrile (65 mg, 0.37 mmol) in THF (3 mL) was added (Boc)₂O (159 mug, 0.73 mmol) at 0° C. followed by Raney Ni (30 mg) and the reaction mixture was degassed under N₂ atmosphere for three times and stirred under a H₂ balloon at room temperature for 4 hours. The mixture was filtered and concentrated to dryness under reduced pressure. The residue was purified by pre-TLC (DCM:MeOH=20:1) to give tert-butyl ((7-oxo-4,5,6,7-tetrahydrothieno[2,3-c]pyridin-2-yl)methyl)carbamate (49 mg, yield 47.6%) as a light oil. LC/MS (ESI) m/z: 283 (M+H)⁺.

### Step 5: tert-butyl ((7-thioxo-4,5,6,7-tetrahydrothieno[2,3-c]pyridin-2-yl)methyl) carbamate

To a solution of ten-butyl ((7-oxo-4,5,6,7-tetrahydrothieno[2,3-c]pyridin-2-yl)methyl)carbamate (49 mg, 0.17 mmol) in THF(3 mL) was added Lawesson's Reagent (140 mg, 0.35 mmol) and the mixture was stirred at room temperature for 3 hours. The mixture was concentrated to dryness. The residue was purified by prep-TLC (DCM:MeOH=20:1) to give tert-butyl ((7-thioxo-4,5,6,7-tetrahydrothieno[2,3-c]pyridin-2-yl)methyl)carbamate (25 mg, yield 48.3%) as a light oil. LC/MS (ESI) m/z: 299 (M+H)⁺.

### Step 6: tert-butyl ((7-(methylthio)-4,5-dihydrothieno[2,3-c]pyridin-2-yl)methyl) carbamate

To a solution of tert-butyl ((7-thioxo-4,5,6,7-tetrahydrothieno [2,3-c]pyridin-2-yl)methyl)carbamate (25 mg, 0.084 mmol) in DMF (2 mL) was added MeI (0.4 mL) and the mixture was stirred at 0° C. for 2 hours. The mixture was concentrated to dryness under reduced pressure to give tert-butyl ((7-(methylthio)-4,5-dihydrothieno[2,3-c]pyridin-2-yl)methyl)carbamate (26 mg, yield 99.3%) as a yellow oil, which was directly used in the next reaction without further purification. LC/MS (ESI) m/z: 313 (M+H)⁺.

### Step 7: tert-butyl ((7-amino-4,5-dihydrothieno[2,3-c]pyridin-2-yl)methyl)carbamate

To a solution of tert-butyl ((7-(methylthio)-4,5-dihydrothieno[2,3-c]pyridin-2-yl)methyl)carbamate (26 mg, 0.083 mmol) in DMF (2 mL) was added NH₃MeOH (1 mL, 7 M), the mixture was stirred at 50° C. for 24 hours. The mixture was concentrated to dryness. The residue was purified by prep-TLC (DCM:MeOH=10:1) to give tert-butyl ((7-amino-4,5-dihydrothieno[2,3-c]pyridin-2-yl)methyl)carbamate (20 mg, yield 85.4%) as a light oil. LC/MS (ESI) m/z: 282 (M+H)⁺.

### Step 8: 2-(aminomethyl)-4,7-dihydrothieno[2,3-c]pyridin-7-amine hydrochloride

A solution of tert-butyl ((7-amino-4,5-dihydrothieno[2,3-c]pyridin-2-yl)methyl) carbamate (20 mg, 0.071 mmol) in HCl/1,4-dioxane (2 mL, 4 M) was stirred under N₂ atmosphere at room temperature for 4 hours. The reaction mixture was concentrated to dryness under reduced pressure, co-evaporated with DCM and dried under vacuum to give 2-(aminomethyl)-4,7-dihydrothieno[2,3-c]pyridin-7-amine hydrochloride (15 mg, yield 96.9%) as a yellow solid, which was used directly in the next reaction without further purification. LC/MS (ESI) mi/z: 182 (M+H)⁺.

### Step 9: (1S,3,55S)-N-((7-amino-4,5-dihydrothieno[2,3-c]pyridin-2-yl)methyl)-5-methyl-2-((4-phenoxybenzoyl)glycyl)-2-azabicyclo[3.1.0]hexane-3-carboxamide (Compound 1)

To a mixture of 2-(aminomethyl)-4,7-dihydrothieno[2,3-c]pyridin-7-amine hydrochloride (15 mg, 0.069 mmol) and (1S,3S,5S)-5-methyl-2-((4-phenoxybenzoyl)glycyl)-2-azabicyclo[3.1.0]hexane-3-carboxylic acid (27 mg, 0.069 mmol) in DMF (2 mL) was added DIPEA (45 mg, 0.68 mmol) and HATU (31 mg, 0.083 mmol) at MC and the mixture was stirred under N₂ atmosphere at room temperature for 1 hour. The mixture was diluted with EtOAc, washed with water and brine, dried over anhydrous Na₂SO₄, filtered, and concentrated to dryness under reduced pressure. The residue was purified by prep-TLC (DCM:MOH 10:1) and further purified by prep-HPLC to give Compound 1 (3.0 mg, yield 7.8%) as a white solid. ¹H NMR (400 MHz, CD₃D): δ 7.88-7.78 (m, 2H), 7.43 (t, J=8.0 Hz, 2H), 7.21 (t, J=7.6 Hz, 1H), 7.11-7.02 (m, 3H),7.02-6.95 (m, 2H), 4.84-4.81 (m, 1H), 4.68-4.57 (m, 2H), 4.44-4.27 (m, 2H), 3.61-3.53 (m, 2H), 3.46-3.36 (m, 1H), 3.00-2.86 (m, 2H), 2.59-2.39 (m, 1H), 2.24-2.05 (m, 1H), 1.31 (s, 3H), 1.19-0.99 (m, 1H), 0.92-0.79 (m, 1H). LC/MS (ESI) m/z: 558 (M+H)⁺. RT (Method A): 1.29 min.

The following compounds were prepared based on Step 9 in Scheme 1:

| Cmpd | | |
|---|---|---|
| # | Reactant A | Reactant B |
| 2 | 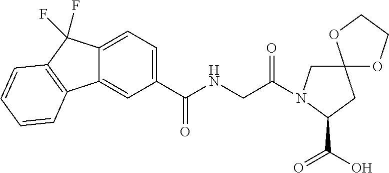 [SMILES: O=C(NCC(=O)N1CC2(C[C@H]1C(=O)O)OCCO2)c1ccc2c(c1)-c1ccccc1C2(F)F] | 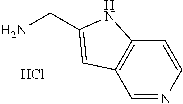 [SMILES: NCc1cc2cnccc2[nH]1 ; Cl] |
| 3 | 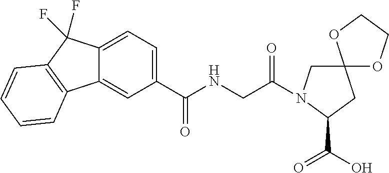 [SMILES: O=C(NCC(=O)N1CC2(C[C@H]1C(=O)O)OCCO2)c1ccc2c(c1)-c1ccccc1C2(F)F] | 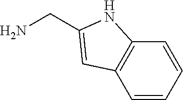 [SMILES: NCc1cc2ccccc2[nH]1] |
| 7 | 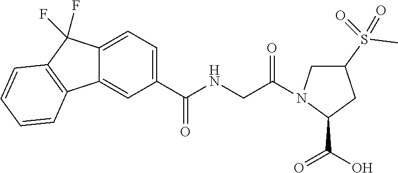 [SMILES: CS(=O)(=O)C1C[C@@H](C(=O)O)N(C(=O)CNC(=O)c2ccc3c(c2)-c2ccccc2C3(F)F)C1] | 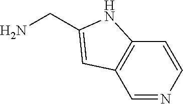 [SMILES: NCc1cc2cnccc2[nH]1] |
| 8 | 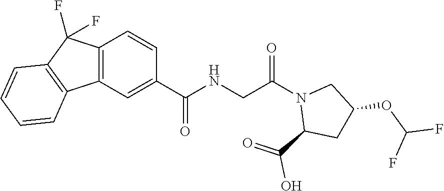 [SMILES: O=C(NCC(=O)N1C[C@H](OC(F)F)C[C@H]1C(=O)O)c1ccc2c(c1)-c1ccccc1C2(F)F] | 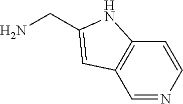 [SMILES: NCc1cc2cnccc2[nH]1] |
| 11 | 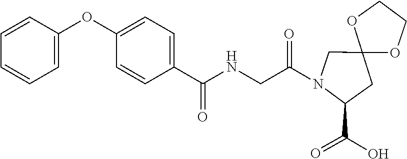 [SMILES: O=C(NCC(=O)N1CC2(C[C@H]1C(=O)O)OCCO2)c1ccc(Oc2ccccc2)cc1] | 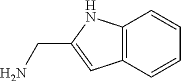 [SMILES: NCc1cc2ccccc2[nH]1] |
| 12 | 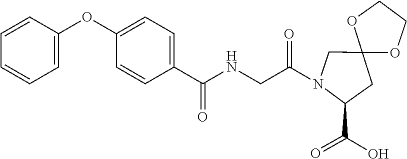 [SMILES: O=C(NCC(=O)N1CC2(C[C@H]1C(=O)O)OCCO2)c1ccc(Oc2ccccc2)cc1] | 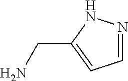 |
| 13 | 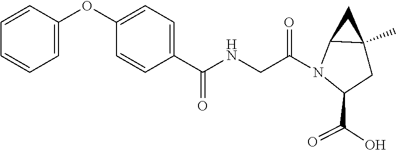 [SMILES: C[C@@]12C[C@@H]1N(C(=O)CNC(=O)c1ccc(Oc3ccccc3)cc1)[C@H](C(=O)O)C2] | 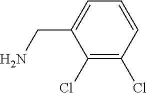 |
| 14 | 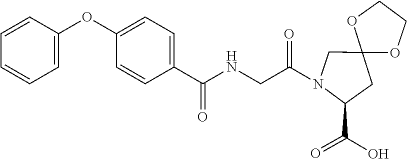 [SMILES: O=C(NCC(=O)N1CC2(C[C@H]1C(=O)O)OCCO2)c1ccc(Oc2ccccc2)cc1] | 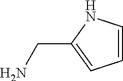 |
| 15 | 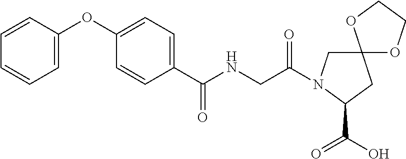 [SMILES: O=C(NCC(=O)N1CC2(C[C@H]1C(=O)O)OCCO2)c1ccc(Oc2ccccc2)cc1] | 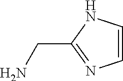 |
| 17 | 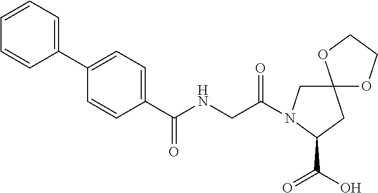 [SMILES: O=C(NCC(=O)N1CC2(C[C@H]1C(=O)O)OCCO2)c1ccc(-c2ccccc2)cc1] | 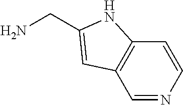 [SMILES: NCc1cc2cnccc2[nH]1] |
| 18 | 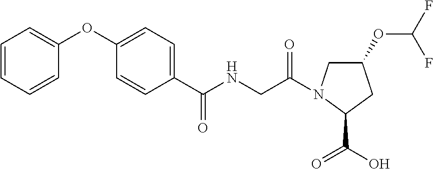 [SMILES: O=C(NCC(=O)N1C[C@H](OC(F)F)C[C@H]1C(=O)O)c1ccc(Oc2ccccc2)cc1] | 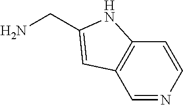 [SMILES: NCc1cc2cnccc2[nH]1] |
| 19 | 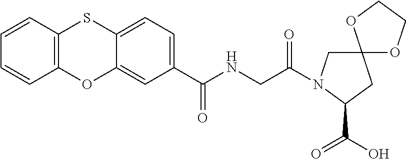 [SMILES: O=C(NCC(=O)N1CC2(C[C@H]1C(=O)O)OCCO2)c1ccc2c(c1)Oc1ccccc1S2] | 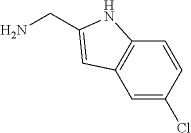 [SMILES: NCc1cc2cc(Cl)ccc2[nH]1] |
| 20 | 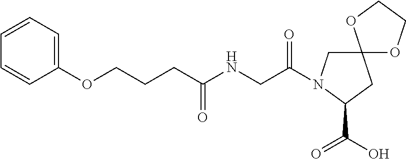 [SMILES: O=C(CCCOc1ccccc1)NCC(=O)N1CC2(C[C@H]1C(=O)O)OCCO2] | 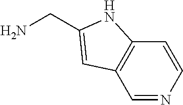 [SMILES: NCc1cc2cnccc2[nH]1] |
| 24 | 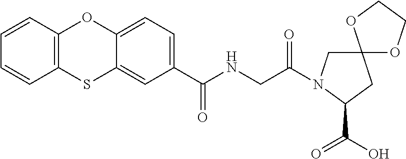 [SMILES: O=C(NCC(=O)N1CC2(C[C@H]1C(=O)O)OCCO2)c1ccc2c(c1)Sc1ccccc1O2] | 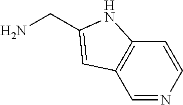 [SMILES: NCc1cc2cnccc2[nH]1] |
| 28 | 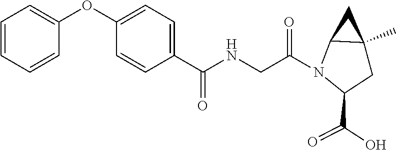 [SMILES: C[C@@]12C[C@@H]1N(C(=O)CNC(=O)c1ccc(Oc3ccccc3)cc1)[C@H](C(=O)O)C2] | 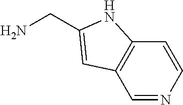 [SMILES: NCc1cc2cnccc2[nH]1] |
| 29 | 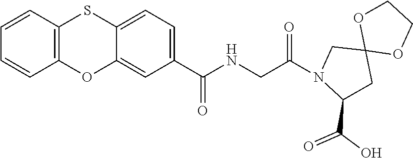 [SMILES: O=C(NCC(=O)N1CC2(C[C@H]1C(=O)O)OCCO2)c1ccc2c(c1)Oc1ccccc1S2] | 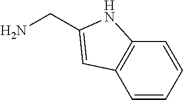 [SMILES: NCc1cc2ccccc2[nH]1] |
| 36 | 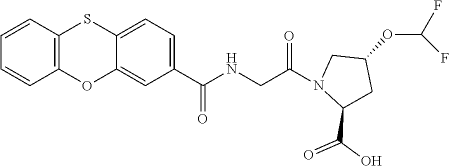 [SMILES: O=C(NCC(=O)N1C[C@H](OC(F)F)C[C@H]1C(=O)O)c1ccc2c(c1)Oc1ccccc1S2] | 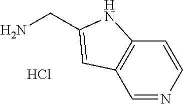 [SMILES: NCc1cc2cnccc2[nH]1 ; Cl] |
| 40 | 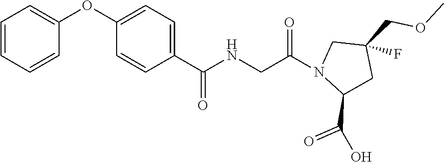 [SMILES: COC[C@@]1(F)C[C@@H](C(=O)O)N(C(=O)CNC(=O)c2ccc(Oc3ccccc3)cc2)C1] | 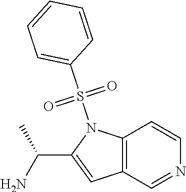 [SMILES: C[C@@H](N)c1cc2cnccc2n1S(=O)(=O)c1ccccc1] |
| 44 | 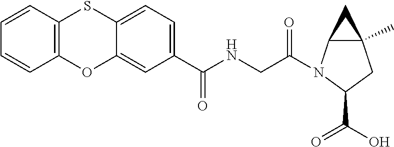 [SMILES: C[C@@]12C[C@@H]1N(C(=O)CNC(=O)c1ccc3c(c1)Oc1ccccc1S3)[C@H](C(=O)O)C2] | 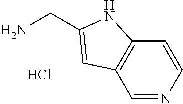 [SMILES: NCc1cc2cnccc2[nH]1 ; Cl] |
| 58^{a} | 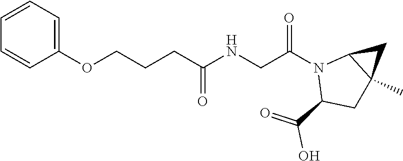 [SMILES: C[C@@]12C[C@@H]1N(C(=O)CNC(=O)CCCOc1ccccc1)[C@H](C(=O)O)C2] | 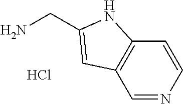 [SMILES: NCc1cc2cnccc2[nH]1 ; Cl] |
| 76^{a} | 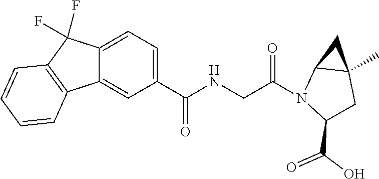 [SMILES: C[C@@]12C[C@@H]1N(C(=O)CNC(=O)c1ccc3c(c1)-c1ccccc1C3(F)F)[C@H](C(=O)O)C2] | 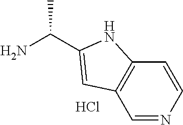 |
| 77^{a} | 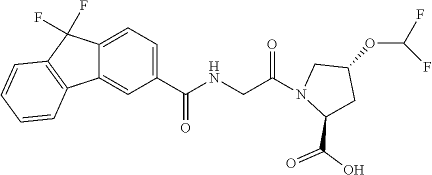 [SMILES: O=C(NCC(=O)N1C[C@H](OC(F)F)C[C@H]1C(=O)O)c1ccc2c(c1)-c1ccccc1C2(F)F] | 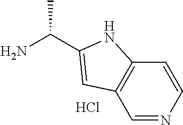 |
| 91^{b} | 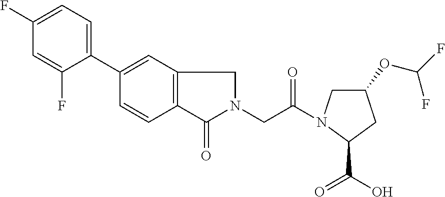 [SMILES: O=C(O)[C@@H]1C[C@@H](OC(F)F)CN1C(=O)CN1Cc2cc(-c3ccc(F)cc3F)ccc2C1=O] | 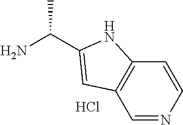 |
| 95 | 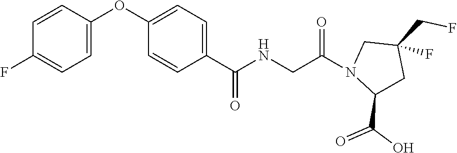 [SMILES: O=C(NCC(=O)N1C[C@@](F)(CF)C[C@H]1C(=O)O)c1ccc(Oc2ccc(F)cc2)cc1] | 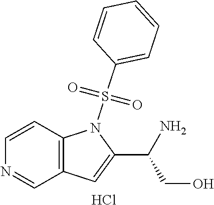 [SMILES: N[C@H](CO)c1cc2cnccc2n1S(=O)(=O)c1ccccc1 ; Cl] |
| 110^{a} | 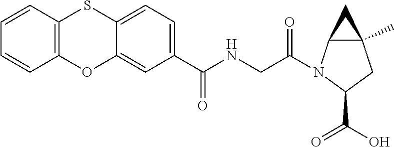 [SMILES: C[C@@]12C[C@@H]1N(C(=O)CNC(=O)c1ccc3c(c1)Oc1ccccc1S3)[C@H](C(=O)O)C2] | 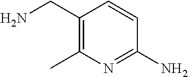 |
| 114^{b} | 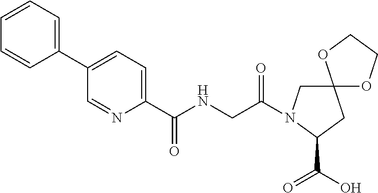 [SMILES: O=C(NCC(=O)N1CC2(C[C@H]1C(=O)O)OCCO2)c1ccc(-c2ccccc2)cn1] | 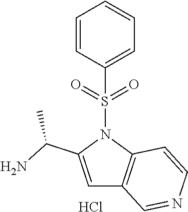 [SMILES: C[C@@H](N)c1cc2cnccc2n1S(=O)(=O)c1ccccc1 ; Cl] |
| 121^{b} | 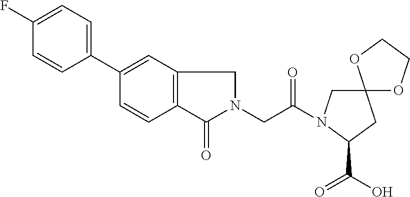 [SMILES: O=C(O)[C@@H]1CC2(CN1C(=O)CN1Cc3cc(-c4ccc(F)cc4)ccc3C1=O)OCCO2] | 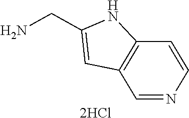 [SMILES: NCc1cc2cnccc2[nH]1 ; "2HCl"] |
| 139^{b} | 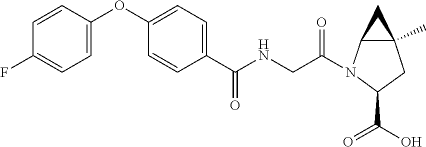 [SMILES: C[C@@]12C[C@@H]1N(C(=O)CNC(=O)c1ccc(Oc3ccc(F)cc3)cc1)[C@H](C(=O)O)C2] | 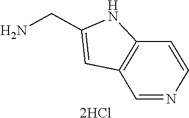 [SMILES: NCc1cc2cnccc2[nH]1 ; "2HCl"] |
| 141 | 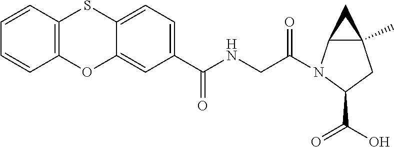 [SMILES: C[C@@]12C[C@@H]1N(C(=O)CNC(=O)c1ccc3c(c1)Oc1ccccc1S3)[C@H](C(=O)O)C2] | 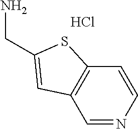 [SMILES: NCc1cc2cnccc2s1 ; Cl] |
| 192 | 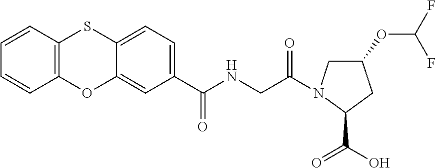 [SMILES: O=C(NCC(=O)N1C[C@H](OC(F)F)C[C@H]1C(=O)O)c1ccc2c(c1)Oc1ccccc1S2] | 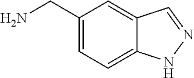 [SMILES: NCc1ccc2[nH]ncc2c1] |
| 193 | 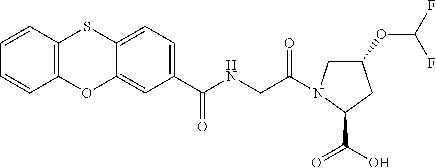 [SMILES: O=C(NCC(=O)N1C[C@H](OC(F)F)C[C@H]1C(=O)O)c1ccc2c(c1)Oc1ccccc1S2] | 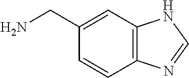 [SMILES: NCc1ccc2nc[nH]c2c1] |
| 195 | 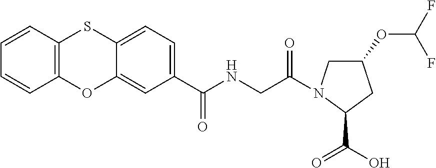 [SMILES: O=C(NCC(=O)N1C[C@H](OC(F)F)C[C@H]1C(=O)O)c1ccc2c(c1)Oc1ccccc1S2] | 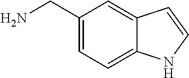 [SMILES: NCc1ccc2[nH]ccc2c1] |
| 196 | 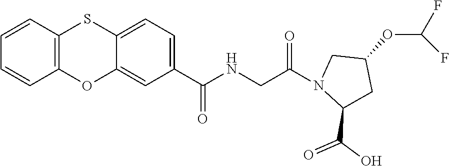 [SMILES: O=C(NCC(=O)N1C[C@H](OC(F)F)C[C@H]1C(=O)O)c1ccc2c(c1)Oc1ccccc1S2] | 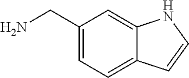 [SMILES: NCc1ccc2cc[nH]c2c1] |
| 199 | 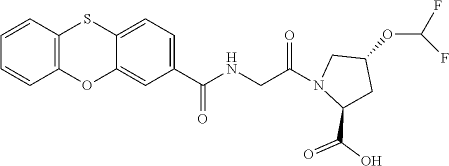 [SMILES: O=C(NCC(=O)N1C[C@H](OC(F)F)C[C@H]1C(=O)O)c1ccc2c(c1)Oc1ccccc1S2] | 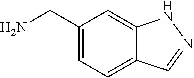 [SMILES: NCc1ccc2cn[nH]c2c1] |
| 201 | 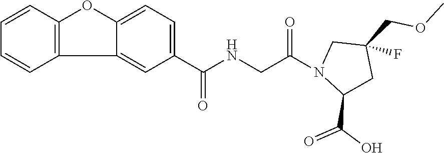 [SMILES: COC[C@@]1(F)C[C@@H](C(=O)O)N(C(=O)CNC(=O)c2ccc3oc4ccccc4c3c2)C1] | 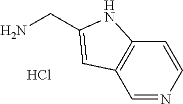 [SMILES: NCc1cc2cnccc2[nH]1 ; Cl] |
| 202 | 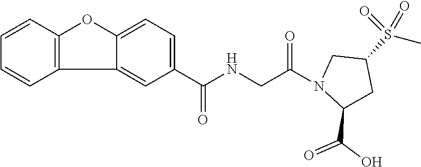 [SMILES: CS(=O)(=O)[C@@H]1C[C@@H](C(=O)O)N(C(=O)CNC(=O)c2ccc3oc4ccccc4c3c2)C1] | 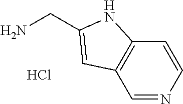 [SMILES: NCc1cc2cnccc2[nH]1 ; Cl] |
| 206 | 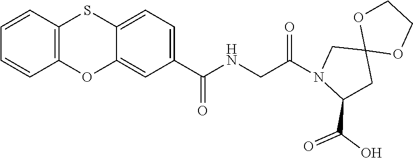 [SMILES: O=C(NCC(=O)N1CC2(C[C@H]1C(=O)O)OCCO2)c1ccc2c(c1)Oc1ccccc1S2] | 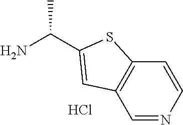 |
| 207 | 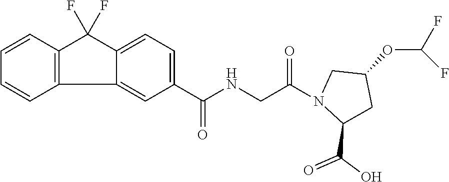 [SMILES: O=C(NCC(=O)N1C[C@H](OC(F)F)C[C@H]1C(=O)O)c1ccc2c(c1)-c1ccccc1C2(F)F] | 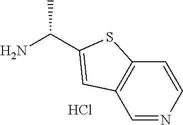 |
| 208 | 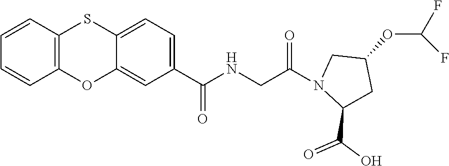 [SMILES: O=C(NCC(=O)N1C[C@H](OC(F)F)C[C@H]1C(=O)O)c1ccc2c(c1)Oc1ccccc1S2] | 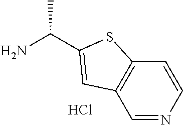 |
| 209 | 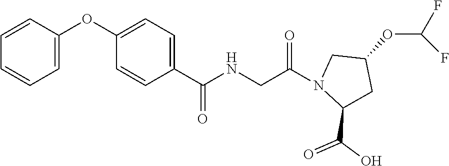 [SMILES: O=C(NCC(=O)N1C[C@H](OC(F)F)C[C@H]1C(=O)O)c1ccc(Oc2ccccc2)cc1] | 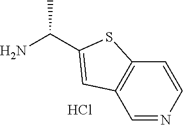 |
| 212 | 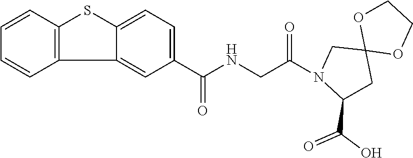 [SMILES: O=C(NCC(=O)N1CC2(C[C@H]1C(=O)O)OCCO2)c1ccc2sc3ccccc3c2c1] | 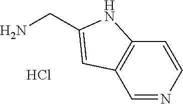 [SMILES: NCc1cc2cnccc2[nH]1 ; Cl] |
| 221 | 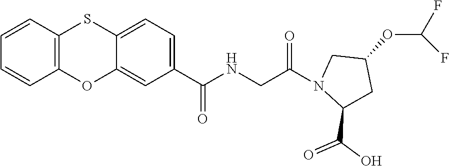 [SMILES: O=C(NCC(=O)N1C[C@H](OC(F)F)C[C@H]1C(=O)O)c1ccc2c(c1)Oc1ccccc1S2] | 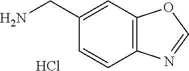 [SMILES: NCc1ccc2ncoc2c1 ; Cl] |
| 231 | 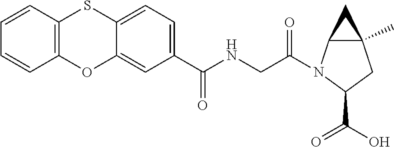 [SMILES: C[C@@]12C[C@@H]1N(C(=O)CNC(=O)c1ccc3c(c1)Oc1ccccc1S3)[C@H](C(=O)O)C2] | 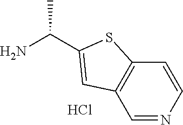 |
| 233 | 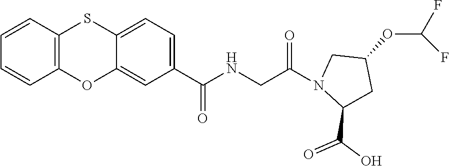 [SMILES: O=C(NCC(=O)N1C[C@H](OC(F)F)C[C@H]1C(=O)O)c1ccc2c(c1)Oc1ccccc1S2] | 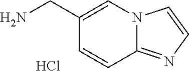 [SMILES: NCc1ccc2nccn2c1 ; Cl] |
| 247 | 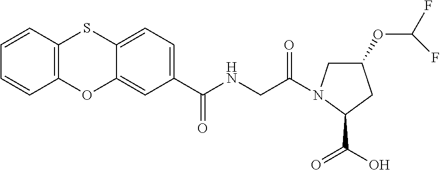 [SMILES: O=C(NCC(=O)N1C[C@H](OC(F)F)C[C@H]1C(=O)O)c1ccc2c(c1)Oc1ccccc1S2] | 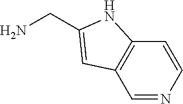 [SMILES: NCc1cc2cnccc2[nH]1] |
| 250 | 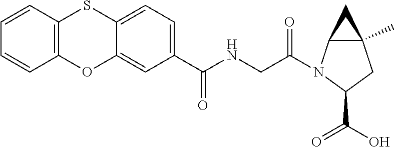 [SMILES: C[C@@]12C[C@@H]1N(C(=O)CNC(=O)c1ccc3c(c1)Oc1ccccc1S3)[C@H](C(=O)O)C2] | 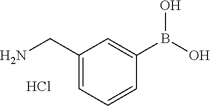 [SMILES: NCc1cccc(B(O)O)c1 ; Cl] |
| 263 | 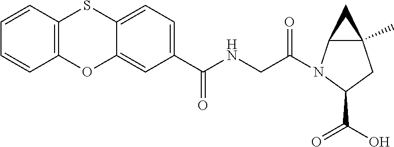 [SMILES: C[C@@]12C[C@@H]1N(C(=O)CNC(=O)c1ccc3c(c1)Oc1ccccc1S3)[C@H](C(=O)O)C2] | 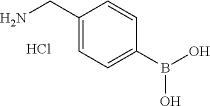 [SMILES: NCc1ccc(B(O)O)cc1 ; Cl] |
| 269 | 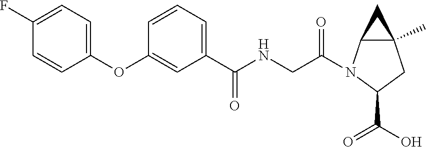 [SMILES: C[C@@]12C[C@@H]1N(C(=O)CNC(=O)c1cccc(Oc3ccc(F)cc3)c1)[C@H](C(=O)O)C2] | 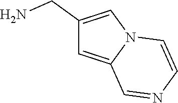 [SMILES: NCc1cc2cnccn2c1] |
| 270 | 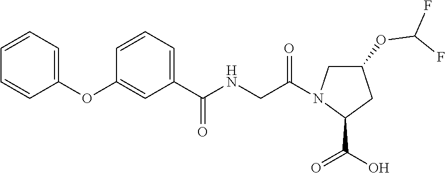 [SMILES: O=C(NCC(=O)N1C[C@H](OC(F)F)C[C@H]1C(=O)O)c1cccc(Oc2ccccc2)c1] | 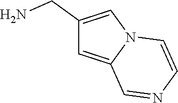 [SMILES: NCc1cc2cnccn2c1] |
| 271 | 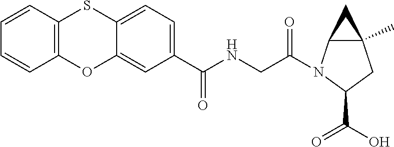 [SMILES: C[C@@]12C[C@@H]1N(C(=O)CNC(=O)c1ccc3c(c1)Oc1ccccc1S3)[C@H](C(=O)O)C2] | 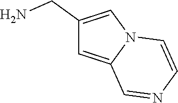 [SMILES: NCc1cc2cnccn2c1] |
| 277 | 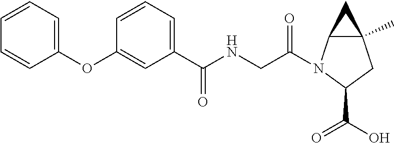 [SMILES: C[C@@]12C[C@@H]1N(C(=O)CNC(=O)c1cccc(Oc3ccccc3)c1)[C@H](C(=O)O)C2] | 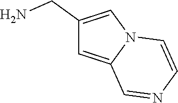 [SMILES: NCc1cc2cnccn2c1] |
| 278 | 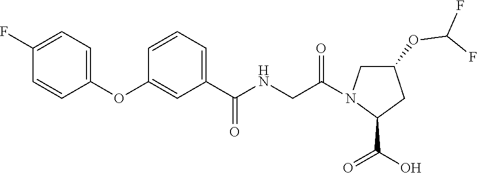 [SMILES: O=C(NCC(=O)N1C[C@H](OC(F)F)C[C@H]1C(=O)O)c1cccc(Oc2ccc(F)cc2)c1] | 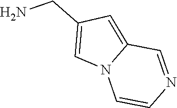 [SMILES: NCc1cc2cnccn2c1] |
| 280 | 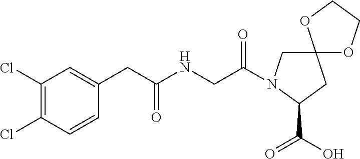 [SMILES: O=C(Cc1ccc(Cl)c(Cl)c1)NCC(=O)N1CC2(C[C@H]1C(=O)O)OCCO2] | 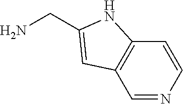 [SMILES: NCc1cc2cnccc2[nH]1] |
| 281 | 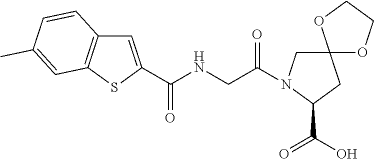 [SMILES: Cc1ccc2cc(C(=O)NCC(=O)N3CC4(C[C@H]3C(=O)O)OCCO4)sc2c1] | 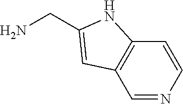 [SMILES: NCc1cc2cnccc2[nH]1] |
| 286 | 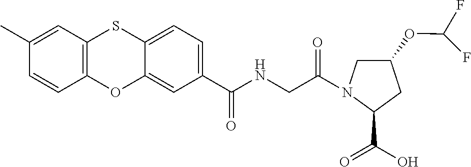 [SMILES: Cc1ccc2c(c1)Sc1ccc(C(=O)NCC(=O)N3C[C@H](OC(F)F)C[C@H]3C(=O)O)cc1O2] | 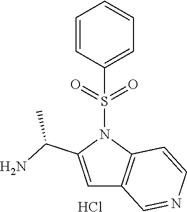 [SMILES: C[C@@H](N)c1cc2cnccc2n1S(=O)(=O)c1ccccc1 ; Cl] |
| 373^{b} | 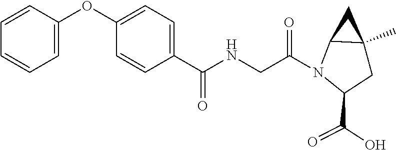 [SMILES: C[C@@]12C[C@@H]1N(C(=O)CNC(=O)c1ccc(Oc3ccccc3)cc1)[C@H](C(=O)O)C2] | 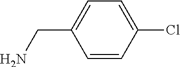 |

| Cmpd # | Characterization Data |
|---|---|
| 2 | ¹H NMR (400 MHz, CD₃OD): δ 8.67 (d, J = 44.7 Hz, 1H), 8.10 (s, 1H), 7.93 |
| | (d, J = 5.9 Hz, 1H), 7.83 (dd, J = 7.8, 1.5 Hz, 1H), 7.67 (dd, J = 15.9, 7.7 Hz, |
| | 3H), 7.57 (t, J = 7.3 Hz, 1H), 7.45 (t, J = 7.6 Hz, 1H), 7.24 (d, J = 5.9 Hz, |
| | 1H), 6.60 (d, J = 26.9 Hz, 1H), 4.64 (d, J = 7.9 Hz, 2H), 4.59 (d, J = 4.1 Hz, |
| | 1H), 4.26 (d, J = 16.6 Hz, 1H), 4.20 (d, J = 16.8 Hz, 1h), 4.04-4.00 (m, 4H), |
| | 3.89-3.81 (m, 2H), 2.47 (dd, J = 13.4, 8.9 Hz, 1H), 2.29 (dd, J = 13.2, 6.1 |
| | Hz, 1H). LC/MS (ESI) (m/z): 588 (M + H)⁺. RT (Method A): 1.22 min |
| 3 | ¹H NMR (400 MHz, CD₃OD): δ 8.12 (s, 1H), 7.86-7.79 (m, 1H), 7.72-7.63 |
| | (m, 3H), 7.56 (t, J = 7.5 Hz, 1H), 7.44 (t, J = 7.2 Hz, 1H), 7.34 (t, J = 11.8 Hz, |
| | 1H), 7.18 (d, J = 7.7 Hz, 1H), 6.89 (ddd, J = 12.6, 9.0, 6.7 Hz, 2H), 6.34 (d, J = |
| | 25.8 Hz, 1H), 4.62 (t, J = 7.5 Hz, 2H), 4.54 (d, J = 15.6 Hz, 1H), 4.22 (s, |
| | 2H), 4.02-3.97 (m, 4H), 3.83 (s, 2H), 2.45 (dd, J = 13.3, 9.0 Hz, 1H), 2.28 |
| | (dd, J = 13.1, 6.2 Hz, 1H). LC/MS (ESI) (m/z): 587 (M + H)⁺. RT (Method A): |
| | 2.29 min. |
| 7 | ¹H NMR (400 MHz, CD₃OD): δ 8.93-8.77 (m, 1H), 8.43 (s, 1H), 8.10 (s, |
| | 1H), 8.03 (d, J = 6.4 Hz, 1H), 7.84 (dd, J = 7.8, 1.4 Hz, 1H), 7.73-7.62 (m, |
| | 4H), 7.59-7.53 (m, 2H), 7.46 (d, J = 7.5 Hz, 1H), 6.80 (d, J = 21.7 Hz, 1H), |
| | 4.76 (d, J = 3.4 Hz, 1H), 4.38 (d, J = 16.5 Hz, 1H), 4.22 (ddd, J = 25.6, 16.3, |
| | 10.5 Hz, 5H), 3.10 (s, 3H), 2.85-2.77 (m, 1H), 2.53-2.46 (m, 1H). LC/MS |
| | (ESI) m/z: 608 (M + H)⁺. RT (Method A): 1.09 min. |
| 8 | ¹H NMR (400 MHz, CD₃OD): δ 8.89 (d, J = 69.9 Hz, 1H), 8.08 (s, 1H), 8.01 |
| | (d, J = 6.6 Hz, 1H), 7.84 (dd, J = 7.8, 1.5 Hz, 1H), 7.67 (dd, J = 15.5, 5.7 Hz, |
| | 3H), 7.57 (dd, J = 9.1, 7.1 Hz, 2H), 7.45 (t, J = 7.5 Hz, 1H), 6.89 (t, J = 19.3 |
| | Hz, 1H), 6.54 (t, J = 74.5 Hz, 1H), 5.07 (s, 1H), 4.73-4.61 (m, 3H), 4.32- |
| | 4.22 (m, 2H), 4.06 (dd, J = 11.6, 4.4 Hz, 1H), 3.94 (d, J = 11.7 Hz, 1H), 2.58- |
| | 2.50 (m, 1H), 2.31 (ddd, J = 13.4, 8.2, 4.8 Hz, 1H). LC/MS (ESI) m/z: 596 |
| | (M + H)⁺. RT (Method A): 1.29 min. |
| 11 | LC/MS (ESI) m/z: 555 (M + H)⁺. RT (Method A): 2.21 min. |
| 12 | LC/MS (ESI) m/z: 506 (M + H)⁺. RT (Method A): 1.52 min. |
| 13 | LC/MS (ESI) m/z: 552 (M + H)⁺. RT (Method A): 2.63 min. |
| 14 | LC/MS (ESI) m/z: 505 (M + H)⁺. RT (Method A): 1.86 min. |
| 15 | LC/MS (ESI) m/z: 506 (M + H)⁺. RT (Method A): 1.02 min. |
| 17 | ¹H NMR (400 MHz, CD₃OD): δ 8.95 (d, J = 56.9 Hz, 1H), 8.17 (dd, J = 67.4, |
| | 6.4 Hz, 1H), 7.87 (d, J = 8.4 Hz, 2H), 7.70-7.64 (m, 5H), 7.48 (t, J = 7.5 Hz, |
| | 2H), 7.40 (t, J = 7.4 Hz, 1H), 6.96 (d, J = 39.8 Hz, 1H), 4.76-4.69 (m, 2H), |
| | 4.66-4.63 (m, 1H), 4.28 (d, J = 16.6 Hz, 1H), 4.16-4.11 (m, 1H), 3.99 (d, J = |
| | 28.8 Hz, 4H), 3.86 (q, J = 10.8 Hz, 2H), 2.49 (dd, J = 12.9, 9.1 Hz, 1H), |
| | 2.28 (dd, J − 13.2, 6.1 Hz, 1H). LC/MS (ESI) m/z: 540 (M + H)⁺. RT (Method |
| | A): 1.21 min. |
| 18 | ¹H NMR (400 MHz, CD₃OD): δ 8.94 (s, 1H), 8.17 (d, J = 6.7 Hz, 1H), 7.84 |
| | (dd, J = 6.9, 1.9 Hz, 2H), 7.72 (d, J = 6.6 Hz, 1H), 7.43 (t, J = 7.9 Hz, 2H), |
| | 7.22 (t, J = 7.4 Hz, 1H), 7.07 (d, J = 8.6 Hz, 2H), 7.01-6.96 (m, 2H), 6.93 (s, |
| | 1H), 6.54 (t, J = 74.4 Hz, 1H), 5.06 (s, 1H), 4.75 (d, J = 16.5 Hz, 1H), 4.63 |
| | (dd, J = 17.6, 9.7 Hz, 2H), 4.24 (s, 2H), 4.02 (dd, J = 11.6, 4.4 Hz, 1H), 3.92 |
| | (d, J = 11.7 Hz, 1H), 2.57-2.48 (m, 1H), 2.35-2.25 (m, 1H). LC/MS (ESI) |
| | m/z: 564 (M + H)⁺. RT (Method A): 1.38 min. |
| 19 | ¹H NMR (400 MHz, CD₃OD): δ 7.48-7.43 (m, 2H), 7.32 (d, J = 1.9 Hz, 1H), |
| | 7.20 (s, 1H), 7.17-7.14 (m,, 2H), 7.09 (t, J = 7.6 Hz, 2H), 7.05-7.02 (m, |
| | 1H), 6.87 (dd, J = 8.6, 2.0 Hz, 1H), 6.27 (s, 1H), 4.58 (dd, J = 14.4, 7.5 Hz, |
| | 3H), 4.15 (s, 2H), 4.01 (d, J = 1.6 Hz, 4H), 3.80 (d, J = 3.7 Hz, 2H), 2.43 (dd, |
| | J = 13.2, 8.9 Hz, 1H), 2.26 (dd, J = 13.1, 6.2 Hz, 1H). LC/MS (ESI) m/z: 619 |
| | (M + H)⁺. RT (Method A): 2.64 min. |
| 20 | ¹H NMR (400 MHz, CD₃OD): δ 9.02-8.94 (m, 1H), 8.23 (d, J = 6.7 Hz, 1H), |
| | 7.88-7.83 (m, 1H), 7.21 (t, J − 7.8 Hz, 2H), 6.98-6.91 (m, 1H), 6.87 (t, J − |
| | 9.0 Hz, 3H), 4.71-4.64 (m, 2H), 4.62-4.57 (m, 1H), 4.09-3.92 (m, 9H), |
| | 3.75 (s, 1H), 2.49-2.42 (m, 3H), 2.25 (dd, J = 13.1, 5.7 Hz, 1H), 2.06 (dt, J = |
| | 13.6, 4.3 Hz, 2H). LC/MS (ESI) m/z: 522 (M + H)⁺. RT (Method A): 0.92 min. |
| 24 | ¹H NMR (400 MHz, CD₃OD): δ 8.81 (s, 1H), 8.06 (d, J = 6.5 Hz, 1H), 7.59 |
| | (d, J = 8.5 Hz, 2H), 7.49 (s, 1H), 7.21 (d, J = 7.1 Hz, 1H), 7.16-7.10 (m, |
| | 2H), 7.06-6.99 (m, 2H), 6.81 (s, 1H), 4.77 (s, 1H), 4.63 (dd, J = 9.2, 6.4 Hz, |
| | 2H), 4.22 (d, J = 16.2 Hz, 1H), 4.08 (s, 1H), 4.02 (s, 4H), 3.85 (d, J = 16.1 Hz, |
| | 2H), 2.51-2.45 (m, 1H), 2.28 (dd, J = 13.3, 5.9 Hz, 1H). LC/MS (ESI) m/z: |
| | 586 (M + H)⁺. RT (Method A): 1.30 min. |
| 28 | ¹H NMR (400 MHz, CD₃OD): δ 8.88 (s, 1H), 8.15 (t, J = 5.3 Hz, 1H), 7.86- |
| | 7.78 (m, 2H), 7.64 (t, J = 5.6 Hz, 1H), 7.43 (dd, J = 10.8, 5.2 Hz, 2H), 7.21 (t, |
| | J = 7.4 Hz, 1H), 7.08-6.93 (m, 4H), 6.84 (d, J = 17.6 Hz, 1H), 4.90 (d, J = |
| | 3.7 Hz, 1H), 4.70-4.60 (m, 2H), 4.44-4.31 (m, 2H), 3.43 (dt, J = 12.2, 6.1 |
| | Hz, 1H), 2.49 (ddd, 1H), 2.17 (ddd 1H), 1.33 (d, J = 3.3 Hz, 3H), 1.28-1.22 |
| | (m, 1H), 0.86 (t, J = 5.8 Hz, 1H). LC/MS (ESI) m/z: 524 (M + H)⁺. RT |
| | (Method A): 1.39 min. |
| 29 | ¹H NMR (400 MHz, CD₃OD): δ 7.55-7.43 (m, 2H), 7.34 (t, J = 15.0 Hz, |
| | 1H), 7.23-7.09 (m, 4H), 7.09-6.99 (m, 2H), 6.97-6.84 (m, 2H), 6.33 (d, |
| | 1H), 4.64-4.48 (m, 3H), 4.23-4.06 (m, 2H), 3.95 (d, 4H), 3.74 (d, J = 14.7 |
| | Hz, 2H), 2.44 (dd, 1H), 2.36-2.17 (m, 1H). LC/MS (ESI) m/z: 585 (M + H)⁺. |
| | RT (Method A): 2.47 min. |
| 36 | ¹H NMR (400 MHz, CD₃OD): δ 8.64 (d, J = 32.9 Hz, 1H), 7.95 (d, J − 5.6 Hz, |
| | 1H), 7.57-7.46 (m, 2H), 7.26-7.12 (m, 4H), 7.10-7.01 (m, 2H), 6.71- |
| | 6.32 (m, 2H), 5.04 (s, 1H), 4.69 (d, J = 16.0 Hz, 1H), 4.57 (dd, J = 22.6, 12.2 |
| | Hz, 2H), 4.30-4.12 (m, 2H), 4.02-3.86 (m, 2H), 2.65-2.45 (m, 1H), 2.41- |
| | 2.23 (m, 1H). LC/MS (ESI) m/z: 594 (M + H)⁺. RT (Method A): 1.34 min. |
| 40 | ¹H NMR (400 MHz, CD₃OD): δ 8.84 (d, J = 3.5 Hz, 1H), 8.48 (s, 1H), 7.98- |
| | 7.82 (m, 3H), 7.43 (t, J = 7.8 Hz, 2H), 7.39-7.34 (m, 1H), 7.22 (t, J = 7.3 Hz, |
| | 1H), 7.06 (dd, J = 18.6, 8.4 Hz, 4H), 6.77 (d, J = 3.9 Hz, 1H), 5.32 (q, J = 6.6 |
| | Hz, 1H), 4.62 (dd, J = 9.3, 7.9 Hz, 1H), 4.35 (d, J = 16.8 Hz, 1H) 4.15 (d, J = |
| | 16.9 Hz, 1H), 4.09-3.88 (m, 2H), 3.73 (dd, J = 17.7, 5.1 Hz, 2H), 3.45 (s, |
| | 3H), 2.59-2.48 (m, 1H), 2.33-2.17 (m, 1H), 1.63 (d, J = 7.0 Hz, 3H). |
| | LC/MS (ESI) m/z: 574 (M + H)⁺. RT (Method A): 1.31 min. |
| 44 | ¹H NMR (400 MHz, CD₃OD): δ 8.81 (d, J = 12.0 Hz, 1H), 8.41 (s, 1H), 8.08 |
| | (t, J = 8.0 Hz, 1H), 7.73-7.28 (m, 3H), 7.23-6.99 (m, 5H), 6.77 (s, 1H), 4.90 |
| | (d, J = 3.2 Hz, 1H), 4.65 (s, 2H), 4.34 (d, J = 5.9 Hz, 2H), 3.44 (d, J = 3.9 Hz, |
| | 1H), 2.47 (t, J = 12.6 Hz, 1H), 2.22 (dd, J = 13.3, 3.6 Hz, 1H), 1.32 (s, 3H), |
| | 1.25 (d, J − 3.3 Hz, 1H), 0.88 (t, J − 5.9 Hz, 1H). LC/MS (ESI) m/z: 554 |
| | (M + H)⁺. RT (Method A): 1.43 min. |
| 58^{a} | ¹H NMR (400 MHz, CD₃OD): δ 8.91 (t, J = 9.2 Hz, 1H), 8.43 (s, 1H), 8.20 |
| | (dd, J = 6.3, 3.6 Hz, 1H), 7.74 (dd, J = 11.6, 6.6 Hz, 1H), 7.25-7.16 (m, 2H), |
| | 6.89-6.78 (m, 4H), 4.83 (d, J = 3.4 Hz, 1H), 4.69-4.51 (m, 2H), 4.20 (dd, J = |
| | 35.9, 16.6 Hz, 2H), 3.97 (t, J = 6.3 Hz, 2H), 3.35 (dd, J = 6.0, 2.4 Hz, 1H), 2.50- |
| | 2.38 (m, 3H), 2.23-2.01 (m, 3H), 1.27 (d, J = 17.6 Hz, 3H), 1.21 (dd, J = |
| | 5.6, 2.3 Hz, 1H), 0.87 (dd, J = 33.2, 28.0 Hz, 1H). LC/MS (ESI) (m/z): 490 |
| | (M + H)⁺. RT (Method A): 1.05 min. |
| 76^{a} | ¹H NMR (400 MHz, CD₃OD): δ 8.61 (s, 1H), 8.49 (s, 1H), 7.98 (d, J = 6.1 Hz, |
| | 1H), 7.90 (s, 1H), 7.71 (d, J = 7.9 Hz, 1H), 7.64 (d, J = 7.1 Hz, 1H), 7.61-7.52 |
| | (m, 3H), 7.49-7.42 (m, 2H), 6.73 (d, J = 26.2 Hz, 1H), 5.33 (t, J = 6.6 Hz, |
| | 1H), 4.95 (d, J = 3.4 Hz, 1H), 4.49 (d, J = 16.0 Hz, 1H), 4.23 (d, J = 15.9 Hz, |
| | 1H), 3.52 (dd, J = 5.9, 2.3 Hz, 1H), 2.51 (t, J = 12.2 Hz, 1H), 2.28 (dd, J = 13.5, |
| | 3.4 Hz, 1H), 1.73 (d, J − 7.0 Hz, 3H), 1.34 (s, 3H), 1.20 (dd, J = 5.8, 2.2 Hz, |
| | 1H), 0.94 (t, J = 6.1 Hz, 1H). LC/MS (ESI) m/z: 570 (M + H)⁺. RT (Method |
| | A): 1.35 min. |
| 77^{a} | ¹H NMR (400 MHz, CD₃OD): δ 8.67 (s, 1H), 8.57-8.12 (m, 1H), 8.03-7.86 |
| | (m, 2H), 7.77 (d, J = 7.7 Hz, 1H), 7.65 (d, J = 7.5 Hz, 2H), 7.58 (dd, J = 13.3, |
| | 6.8 Hz, 2H), 7.46 (dd, J = 14.1, 6.7 Hz, 2H), 6.78-6.35 (m, 2H), 5.33 (q, J = |
| | 7.0 Hz, 1H), 5.05 (s, 1H), 4.62 (t, J = 8.1 Hz, 1H), 4.30 (d, J = 16.3 Hz, 1H), |
| | 4.18 (t, J = 12.8 Hz, 1H), 4.10 (dd, J = 11.6, 4.4 Hz, 1H), 3.95 (d, J = 11.6 Hz, |
| | 1H), 2.64-2.46 (m, 1H), 2.37-2.24 (m, 1H), 1.71 (d, J = 7.0 Hz, 3H). |
| | LC/MS (ESI) m/z: 610 (M + H)⁺. RT (Method A): 1.41 min. |
| 91^{b} | ¹H NMR (400 MHz, DMSO) δ 11.61 (s, 1H), 8.84 (d, J = 7.9 Hz, 1H), 8.73 (s, |
| | 1H), 8.45 (d, J = 8.2 Hz, 1H), 8.07 (s, 1H), 8.04 (d, J = 5.8 Hz, 1H), 7.74 (d, J = |
| | 7.9 Hz, 1H), 7.68 (s, 1H), 7.63-7.50 (m, 1H), 7.35 (ddd, J = 13.3, 7.9, 3.3 |
| | Hz, 1H), 7.28-7.22 (m, 1H), 7.18 (td, J = 8.4, 2.5 Hz, 1H), 6.92 (d, J = 12.3 |
| | Hz, 1H), 6.72 (s, 1H), 6.56 (s, 1H), 5.16 (t, J = 7.2 Hz, 1H), 5.05 (p, J = 7.0 |
| | Hz, 1H), 4.89 (s, 1H), 4.83 (s, 1H), 4.63 (t, J = 7.3 Hz, 1H), 4.46 (d, J = 6.8 |
| | Hz, 1H), 4.44-4.29 (m, 2H), 3.80 (dd, J = 11.4, 4.6 Hz, 1H), 3.76-3.67 (m, |
| | 1H), 3.62-3.54 (m, 1H), 2.28 (ddd, J = 12.6, 8.2, 4.0 Hz, 1H), 2.06 (ddd, J = |
| | 13.1, 7.5, 5.0 Hz, 1H), 1.40 (d, J = 6.9 Hz, 3H). LC/MS (ESI) m/z: 610 |
| | (M + H)⁺. RT (Method A): 1.52 min. |
| 95 | ¹H NMR (400 MHz, CD₃OD): δ 8.81 (d, J = 6.1 Hz, 1H), 8.49 (d, J = 6.6 Hz, |
| | 1H), 8.16-7.96 (m, 1H), 7.92-7.81 (m, 2H), 7.46 (dd, J = 89.2, 6.1 Hz, 1H), |
| | 7.17 (dt, J = 14.3, 4.3 Hz, 2H), 7.11 (dd, J = 9.2, 4.5 Hz, 2H), 7.02 (dd, J = |
| | 16.2, 9.3 Hz, 2H), 6.80 (t, J = 9.0 Hz, 1H), 5.29 (t, J = 5.7 Hz, 1H), 4.82-4.74 |
| | (m, 2H), 4.71-4.64 (m, 1H), 4.35 (d, J = 17.0 Hz, 1H), 4.13 (dd, J = 17.6, |
| | 14.5 Hz, 2H), 4.05-3.94 (m, 2H), 3.93 (dd, J = 7.2, 4.0 Hz, 1H), 2.73-2.41 |
| | (m, 1H), 2.39-2.19 (m, 1H). LC/MS (ESI) m/z: 596 (M + H)⁺. RT (Method |
| | A): 1.23 min. |
| 110^{a} | ¹H NMR (400 MHz, CD₃OD): δ 8.33 (s, 1H), 7.68 (d, J = 4.6 Hz, 1H), 7.49 |
| | (dd, J = 6.5, 4.0 Hz, 1H), 7.37 (dd, J = 15.7, 7.7 Hz, 1H), 7.23-7.17 (m, 2H), |
| | 7.15 (d, J = 7.6 Hz, 1H), 7.06 (dd, J = 18.5, 7.8 Hz, 2H), 6.61 (d, J = 8.9 Hz, |
| | 1H), 4.79 (dd, J = 11.4, 3.4 Hz, 1H), 4.31 (d, J = 8.9 Hz, 1H), 4.25 (dd, J = 9.5, |
| | 5.4 Hz, 2H), 3.61 (d, J = 7.0 Hz, 1H), 3.38 (dd, J = 5.9, 2.3 Hz, 1H), 2.47- |
| | 2.37 (m, 4H), 2.14 (dd, J = 13.4, 3.4 Hz, 1H), 1.29 (s, 3H), 1.18 (t, J = 7.0 Hz, |
| | 1H), 0.81 (t, J = 5.7 Hz, 1H). LC/MS (ESI) m/z: 544 (M + H)⁺. RT (Method A): |
| | 1.43 min. |
| 114^{b} | ¹H NMR (400 MHz, DMSO-d6): δ 11.02 (s, 1H), 8.97 (d, J = 2.3 Hz, 1H), |
| | 8.93 (d, J = 2.4 Hz, 1H), 8.86 (t, J = 5.4 Hz, 1H), 8.73 (t, J = 5.2 Hz, 1H), 8.66 |
| | (d, J = 6.4 Hz, 1H), 8.52 (d, J = 8.4 Hz, 1H), 8.35-8.19 (m, 3H), 8.13-7.99 |
| | (m, 2H), 7.87 (d, J = 5.6 Hz, 1H), 7.77 (t, J = 8.6 Hz, 3H), 7.49 (q, J = 7.7 Hz, |
| | 3H), 7.43 (t, J = 7.0 Hz, 1H), 7.04 (d, J = 5.6 Hz, 1H), 6.38 (s, 1H), 5.10 (p, J = |
| | 8.1 Hz, 1H), 4.37 (t, J = 8.0 Hz, 1H), 4.27-4.04 (m, 3H), 4.02-3.81 (m, |
| | 6H), 3.75 (d, J = 10.8 Hz, 1H), 3.58 (d, J = 10.8 Hz, 1H), 2.26 (dd, J = 13.0, |
| | 8.4 Hz, 1H), 2.08 (dd, J = 12.9, 8.0 Hz, 1H). LC/MS (ESI) m/z: 555 (M + H)⁺. |
| | RT (Method A): 1.25 min. |
| 121^{b} | ¹H NMR (400 MHz, DMSO-d6): δ 11.17 (s, 1H), 9.04-8.52 (m, 2H), 8.16 (s, |
| | 1H), 7.99 (s, 1H), 7.86 (s, 1H), 7.85-7.71 (m, 2H), 7.35 (t, J = 8.6 Hz, 1H), |
| | 7.19-7.03 (m, 2H), 6.49 (s, 1H), 6.40 (s, 1H), 4.54 (dd, J = 14.3, 9.9 Hz, 2H), |
| | 4.47-4.26 (m, 2H), 3.96 (qt, J = 9.5, 4.2 Hz, 2H), 3.83 (d, J = 10.7 Hz, 1H), |
| | 3.67 (dd, J = 11.5, 6.4 Hz, 1H), 2.35 (dd, J = 13.1, 8.4 Hz, 1H), 2.11 (dd, J = |
| | 13.1, 7.3 Hz, 1H). LC/MS (ESI) m/z: 570 (M + H)⁺. RT (Method A): 1.32 min. |
| 139^{b} | ¹H NMR (400 MHz, DMSO-d6): δ 11.15 (s, 1H), 8.73-8.62 (m, 1H), 8.60 (s, |
| | 1H), 8.36 (t, J = 5.8 Hz, 1H), 8.25 (s, 1H), 7.96 (d, J = 5.6 Hz, 1H), 7.88 (d, J = |
| | 8.4 Hz, 1H), 7.71 (d, J = 8.5 Hz, 1H), 7.22-6.94 (m, 2H), 6.29 (s, 1H), |
| | 4.65 (dd, J = 11.3, 3.3 Hz, 1H), 4.40 (d, J = 5.7 Hz, 1H), 4.30 (d, J = 5.7 Hz, |
| | 1H), 4.02 (dd, J = 16.5, 5.8 Hz, 1H), 3.46-3.33 (m, 3H), 2.24 (t, J = 12.1 Hz, |
| | 1H), 2.00 (dd, J = 13.2, 3.3 Hz, 1H), 1.08 (s, 3H), 0.99-0.72 (m, 1H), 0.64 (t, |
| | J = 5.6 Hz, 1H). LC/MS (ESI) m/z: 592 (M + H)⁺. RT (Method A): 1.76 min. |
| 141 | ¹H NMR (400 MHz, CD₃OD): δ 8.86 (s, 1H), 8.22 (d, J = 5.7 Hz, 1H), 7.79 |
| | (d, J = 5.3 Hz, 1H), 7.49 (d, J = 8.4 Hz, 1H), 7.41 (s, 2H), 7.22-7.13 (m, 3H), |
| | 7.05 (dd, J = 16.3, 8.0 Hz, 2H), 6.51 (t, J = 74.5 Hz, 1H), 5.02 (s, 1H), 4.77 (s, |
| | 1H), 4.62 (dd, J = 19.6, 11.8 Hz, 2H), 4.19 (s, 2H), 4.00 (d, J = 7.3 Hz, 1H), |
| | 3.88 (d, J = 11.2 Hz, 1H), 2.49 (dd, J = 13.8, 7.7 Hz, 1H), 2.32-2.21 (m, 1H). |
| | LC/MS (ESI) m/z: 571 (M + H)⁺. RT (Method A): 4.20 min. |
| 192 | ¹H NMR (400 MHz, CD₃OD): δ 7.97 (d, J = 17.5 Hz, 1H), 7.71 (d, J = 16.3 |
| | Hz, 1H), 7.52-7.44 (m, 3H), 7.40-7.32 (m, 1H), 7.22-7.13 (m, 3H), 7.09- |
| | 7.02 (m, 2H), 6.48 (td, J = 74.5, 17.2 Hz, 1H), 5.00 (d, J = 3.4 Hz, 1H), 4.59 |
| | (dd, J = 15.2, 7.4 Hz, 1H), 4.55-4.45 (m, 2H), 4.31-4.10 (m, 2H), 3.96 (dd, |
| | J = 11.5, 4.7 Hz, 1H), 3.85 (d, J = 12.7 Hz, 1H), 2.66-2.42 (m, 1H), 2.37- |
| | 2.18 (m, 1H). LC/MS (ESI) m/z: 594 (M + H)⁻. RT (Method A): 2.00 min. |
| 193 | LC/MS (ESI) m/z: 594 (M + H)⁺. RT (Method A): 1.54 min. |
| 195 | ¹H NMR (400 MHz, CD₃OD): δ 7.49 (dd, J = 8.4, 4.6 Hz, 3H), 7.32 (dd, J = |
| | 13.9, 8.4 Hz, 1H), 7.21-7.13 (m, 4H), 7.06 (ddd, = 14.4, 7.8, 2.9 Hz, 3H), |
| | 6.68-6.39 (m, 1H), 6.37-6.26 (m, 1H), 4.95 (d, J = 31.0 Hz, 1H), 4.58 (dd, J − |
| | 15.9, 8.1 Hz, 1H), 4.46 (d, J − 5.1 Hz, 2H), 4.25 (dd, J − 17.0, 4.6 Hz, 1H), |
| | 4.14 (dd, J = 18.5, 8.7 Hz, 1H), 3.94 (d, J = 11.3 Hz, 1H), 3.82 (d, J = 15.6 |
| | Hz, 1H), 2.44 (dd, J − 12.8, 8.9 Hz, 1H), 2.36-2.20 (m, 1H). LC/MS (ESI) |
| | m/z: 593 (M + H)⁺. RT (Method A): 2.23 min. |
| 196 | ¹H NMR (400 MHz, CD₃OD): δ 7.47-7.61 (m, 3H), 7.35 (s, 1H), 7.19-7.11 |
| | (m, 4H), 7.08-6.99 (m, 2H), 6.93 (d, J = 8.1 Hz, 1H), 6.68-6.38 (m, 1H), |
| | 6.36-6.25 (m, 1H), 4.98-4.90 (m, 1H), 4.69-4.56 (m, 1H), 4.53-4.44 (m, |
| | 2H), 4.31-4.03 (m, 2H), 3.97-3.88 (m, 1H), 3.86-3.78 (m, 1H), 2.64-2.41 |
| | (m, 1H), 2.35-2.19 (m, 1H). LC/MS (ESI) m/z: 593 (M + H)⁺. RT (Method |
| | A): 1.60 min. |
| 199 | ¹H NMR (400 MHz, CD₃OD): δ 7.95 (d, J = 24.1 Hz, 1H), 7.69 (dd, J = 28.2, |
| | 8.4 Hz, 1H), 7.52-7.43 (m, 3H), 7.23-7.13 (m, 3H), 7.10-7.02 (m, 3H), |
| | 6.70-6.28 (m, 1H), 4.97 (d, J = 38.4 Hz, 1H), 4.64 (t, J = 7.7 Hz, 1H), 4.53 (t, |
| | J = 9.6 Hz, 2H), 4.29-4.13 (m, 2H), 3.98 (dd, J = 11.5, 4.6 Hz, 1H), 3.87 (d, |
| | J = 11.3 Hz, 1H), 2.63-2.44 (m, 1H), 2.37-2.22 (m, 1H). LC/MS (ESI) m/z: |
| | 594 (M + H)⁺. RT (Method A): 2.05 min. |
| 201 | ¹H NMR (400 MHz, CD₃OD): δ 8.61-8.52 (m, 2H), 8.06-7.99 (m, 2H), 7.82 |
| | (dd, J = 6.2, 1.0 Hz, 1H), 7.65 (d, J = 8.5 Hz, 2H), 7.56 (t, J = 7.8 Hz, 1H), |
| | 7.42 (t, J = 7.5 Hz, 1H), 7.22 (d, J = 5.9 Hz, 1H), 6.59 (s, 1H), 4.74-4.64 (m, |
| | 2H), 4.59 (d, J = 15.5 Hz, 1H), 4.29 (q, J = 16.7 Hz, 2H), 4.11-3.93 (m, 2H), |
| | 3.76-3.70 (m, 2H), 3.45 (s, 3H), 2.65-2.52 (m, 1H), 2.34-2.19 (m, 1H). |
| | LC/MS (ESI) m/z: 588 (M + H)⁺. RT (Method A): 1.27 min. |
| 202 | ¹H NMR (400 MHz, CD₃OD): δ 8.58 (s, 1H), 8.54 (s, 1H), 8.02 (s, 1H), 7.99 |
| | (dd, J = 8.7, 1.8 Hz, 1H), 7.89 (d, J = 5.9 Hz, 1H), 7.62 (t, J = 4.3 Hz, 2H), |
| | 7.55 (s, 1H), 7.43 (d, J = 7.6 Hz, 1H), 7.22 (d, J = 5.8 Hz, 1H), 6.57 (d, J = |
| | 31.4 Hz, 1H), 4.76 (dd, J = 8.6, 5.0 Hz, 1H), 4.63 (d, J = 10.2 Hz, 2H), 4.34 |
| | (d, J = 14.6 Hz, 2H), 4.25-4.19 (m, 3H), 3.10 (s, 3H), 2.79 (dd, J = 10.0, 3.8 |
| | Hz, 1H), 2.53-2.46 (m, 1H). LC/MS (ESI) m/z: 574 (M + H)⁺. RT (Method |
| | A): 1.14 min. |
| 202 | ¹H NMR (400 MHz, CD₃OD): δ 9.25 (d, J = 17.7 Hz, 1H), 8.49-8.35 (m, |
| | 2H), 7.79 (d, J = 9.3 Hz, 1H), 7.53-7.36 (m, 2H), 7.22-7.12 (m, 3H), 7.07 (t, |
| | J = 7.5 Hz, 1H), 7.00 (d, J = 7.8 Hz, 1H), 5.45 (dq, J = 13.6, 6.8 Hz, 1H), 4.72- |
| | 4.56 (m, 1H), 4.15-4.04 (m, 2H), 4.04-3.99 (m, 3H), 3.96 (dd, J = 8.4, 5.6 |
| | Hz, 1H), 3.83-3.74 (m, 2H), 2.50-2.37 (m, 1H), 2.36-2.11 (m, 1H), 1.72 |
| | (dd, J = 32.7, 7.0 Hz, 3H). LC/MS (ESI) m/z: 617 (M + H)⁺. RT (Method A): |
| | 1.60 min. |
| 207 | ¹H NMR (400 MHz, CD₃OD): δ 8.18 (s, 1H), 8.10 (s, 1H), 7.96 (s, 1H), 7.88 |
| | (d, J = 7.8 Hz, 1H), 7.76-7.63 (m, 4H), 7.57 (d, J = 3.8 Hz, 2H), 7.44 (d, J = |
| | 7.6 Hz, 1H), 6.54 (t, J − 74.4 Hz, 1H), 5.39-5.35 (m, 1H), 5.06 (s, 1H), 4.65 |
| | (t, J = 7.6 Hz, 1H), 4.34 (d, J = 16.7 Hz, 1H), 4.19 (d, J = 16.6 Hz, 1H), 4.01- |
| | 3.97 (m, 1H), 3.90 (d, J = 10.9 Hz, 1H), 2.20 (t, J = 7.6 Hz, 1H), 2.05 (s, 1H), |
| | 1.66 (d, J = 7.0 Hz, 3H). LC/MS (ESI) m/z: 627 (M + H)⁺. RT (Method A): |
| | 1.48 min. |
| 208 | ¹H NMR (400 MHz, CD₃OD): δ 8.95 (s, 1H), 8.26 (d, J = 5.6 Hz, 1H), 7.90 |
| | (d, J = 5.8 Hz, 1H), 7.51 (d, J = 13.1 Hz, 2H), 7.46 (d, J = 1.8 Hz, 1H), 7.18 |
| | (d, J = 8.1 Hz, 2H), 7.13 (d, J = 1.6 Hz, 1H), 7.09-7.05 (m, 1H), 7.01 (dd, J = |
| | 8.0, 1.1 Hz, 1H), 6.52 (t, J = 74.5 Hz, 1H), 5.36 (d, J = 6.1 Hz, 1H), 5.03 (s, |
| | 1H), 4.62 (t, J = 7.7 Hz, 1H), 4.25 (d, J = 16.7 Hz, 1H), 4.10 (d, J = 16.7 Hz, |
| | 1H), 3.95 (dd, J = 11.4, 4.8 Hz, 1H), 3.85 (d, J = 12.6 Hz, 1H), 2.66-2.44 (m, |
| | 1H), 2.40-2.22 (m, 1H), 1.67 (dd, J = 26.1, 7.0 Hz, 3H). LC/MS (ESI) m/z: |
| | 625 (M + H)⁺. RT (Method A): 1.61 min. |
| 209 | ¹H NMR (400 MHz, CD₃OD): δ 9.01 (s, 1H), 8.30 (dd, J = 8.6, 5.7 Hz, 1H), |
| | 7.99 (t, J − 8.3 Hz, 1H), 7.82 (dd, J − 24.2, 8.8 Hz, 2H), 7.57 (s, 1H), 7.42 (t, J = |
| | 7.9 Hz, 2H), 7.20 (t, J = 7.4 Hz, 1H), 7.06 (d, J = 8.4 Hz, 2H), 6.99 (d, J = |
| | 8.7 Hz, 2H), 6.50 (td, J = 74.5, 15.4 Hz, 1H), 5.48-5.34 (m, 1H), 5.03 (s, |
| | 1H), 4.62 (t, J = 7.7 Hz, 1H), 4.31-4.08 (m, 2H), 3.97-3.78 (m, 2H), 2.48 |
| | (dd, J = 12.9, 8.8 Hz, 1H), 2.40-2.22 (m, 1H), 1.68 (dd, J = 25.5, 7.0 Hz, |
| | 3H). LC/MS (ESI) m/z: 595 (M + H)⁺. RT (Method A): 1.50 min. |
| 212 | ¹H NMR (400 MHz, CD₃OD): δ 8.59 (s, 1H), 8.53 (s, 1H), 8.15 (d, J = 7.4 Hz, |
| | 1H), 7.97 (dd, J = 28.6, 5.8 Hz, 2H), 7.87-7.80 (m, 3H), 7.53 (t, J = 6.7 Hz, |
| | 2H), 7.46 (d, J = 6.7 Hz, 1H), 6.83 (s, 1H), 4.73-4.61 (m, 3H), 4.33 (d, J = |
| | 16.3 Hz, 1H), 4.15 (s, 1H), 4.05 (s, 4H), 3.97 (d, J = 10.7 Hz, 1H), 3.88 (d, J = |
| | 10.8 Hz, 1H), 2.52 (dd, J = 13.1, 9.1 Hz, 1H), 2.31 (dd, J = 13.3, 5.7 Hz, 1H). |
| | LC/MS (ESI) m/z: 570 (M + H)⁺. RT (Method A): 1.28 min. |
| 221 | ¹H NMR (400 MHz, CD₃OD): δ 8.38-8.30 (m, 1H), 7.65 (t, J − 6.6 Hz, 2H), |
| | 7.51-7.47 (m, 1H), 7.45-7.42 (m, 1H), 7.36 (d, J = 8.3 Hz, 1H), 7.21-7.13 |
| | (m, 3H), 7.09-7.03 (m, 2H), 6.61 (d, J = 74.5 Hz, 1H), 5.03-4.99 (m, 1H), |
| | 4.60 (d, J = 7.7 Hz, 1H), 4.56 (s, 2H), 4.24-4.13 (m, 2H), 4.00-3.95 (m, |
| | 1H), 3.86 (d, J = 11.3 Hz, 1H), 2.52-2.45 (m, 1H), 2.28-2.21 (m, 1H). |
| | LC/MS (ESI) m/z: 595 (M + H)⁺. RT (Method A): 2.10 min. |
| 231 | ¹H NMR (400 MHz, CD₃OD): δ 8.95 (d, J = 0.8 Hz, 1H), 8.27 (d, J = 5.8 Hz, |
| | 1H), 7.87 (d, J = 5.7 Hz, 1H), 7.50-7.46 (m, 3H), 7.21-7.16 (m, 2H), 7.14 |
| | (s, 1H), 7.09-7.07 (m, 1H), 7.02-6.99 (m, 1H), 5.36-5.31 (m, 1H), 4.89 (d, |
| | J = 3.3 Hz, 1H), 4.40-4.35 (m, 1H), 4.27-4.21 (m, 1H), 3.38 (dd, J = 5.8, |
| | 2.1 Hz, 1H), 2.47-2.39 (m, 1H), 2.23-2.17 (m, 1H), 1.65 (d, J = 6.9 Hz, 3H), |
| | 1.30 (s, 3H), 1.19-1.15 (m, 1H), 0.79-0.74 (m, 1H). LC/MS (ESI) m/z: 585 |
| | (M + H)⁺. RT (Method A): 1.65 min. |
| 233 | ¹H NMR (400 MHz, CD₃OD): δ 8.46 (s, 1H), 7.73 (s, 1H), 7.50 (dd, J = 11.5, |
| | 5.3 Hz, 3H), 7.44 (s, 1H), 7.37 (d, J = 8.8 Hz, 1H), 7.21-7.13 (m, 3H), 7.10- |
| | 7.06 (m, 1H), 7.03 (d, J = 7.9 Hz, 1H), 6.52 (t, J = 74.5 Hz, 1H), 5.03 (s, 1H), |
| | 4.59 (t, J − 8.0 Hz, 1H), 4.46 (d, J − 8.4 Hz, 2H), 4.20 (s, 2H), 4.00 (dd, J − |
| | 11.5, 4.5 Hz, 1H), 3.88 (d, J = 11.8 Hz, 1H), 2.55-2.45 (m, 1H), 2.30-2.20 |
| | (m, 1H). LC/MS (ESI) m/z: 594 (M + H)⁺. RT (Method A): 1.45 min. |
| 247 | ¹H NMR (400 MHz, CD₃OD): δ 8.15 (d, J = 1.8 Hz, 1H), 7.96 (d, J = 1.8 Hz, |
| | 1H), 7.53-7.47 (m, 2H), 7.32 (d, J = 3.5 Hz, 1H), 7.22-7.14 (m, 3H), 7.10- |
| | 7.02 (m, 2H), 6.69-6.32 (m, 2H), 5.02-4.97 (m, 1H), 4.58 (d, J = 7.8 Hz, |
| | 1H), 4.51 (d, J = 2.4 Hz, 2H), 4.27-4.11 (m, 2H), 3.95 (dd, J = 11.4, 4.6 Hz, |
| | 1H), 3.85 (d, J = 11.3 Hz, 1H), 2.50-2.43 (m, 1H), 2.25-2.18 (m, 1H). |
| | LC/MS (ESI) m/z: 594 (M + H)⁺. RT (Method A): 1.93 min. |
| 250 | ¹H NMR (400 MHz, CD₃OD): δ 7.54-7.45 (m, 4H), 7.35-7.27 (m, 2H), |
| | 7.23-7.18 (m, 2H), 7.15 (dd, J = 7.7, 1.5 Hz, 1H), 7.06 (dd, J = 15.6, 7.8 Hz, |
| | 2H), 4.83 (d, J = 3.2 Hz, 1H), 4.48-4.33 (m, 3H), 4.29-4.25 (m, 1H), 3.39- |
| | 3.36 (m, 1H), 2.41 (t, J = 12.7 Hz, 1H), 2.20-2.14 (m, 1H), 1.29 (s, 3H), 1.22- |
| | 1.09 (m, 1H), 0.83-0.75 (m, 1H). LC/MS (ESI) m/z: 558 (M + H)⁺. RT |
| | (Method A): 2.06 min. |
| 263 | LC/MS (ESI) m/z: 558 (M + H)⁺. RT (Method A): 1.94 min. |
| 269 | ¹H NMR (400 MHz, CD₃OD): δ 8.82 (s, 1H), 8.23 (d, J = 5.3 Hz, 1H), 7.88 |
| | (d, J = 0.5 Hz, 1H), 7.54 (d, J = 7.7 Hz, 1H), 7.42 (d, J = 8.0 Hz, 1H), 7.40- |
| | 7.37 (m, 2H), 7.17 (s, 1H), 7.14-7.09 (m, 3H), 7.05-7.01 (m, 2H), 4.83 (d, J = |
| | 2.0 Hz, 1H), 4.53 (s, 2H), 4.33 (s, 2H), 3.42-3.39 (m, 1H), 2.49-2.38 (m, |
| | 1H), 2.23-2.16 (m, 1H), 1.30 (s, 3H), 1.21-1.17 (m, 1H), 0.85-0.78 (m, |
| | 1H). LC/MS (ESI) m/z: 542 (M + H)⁺. RT (Method A): 1.41 min. |
| 270 | ¹H NMR (400 MHz, CD₃OD): δ 8.63 (d, J = 15.9 Hz, 1H), 8.02 (dd, J = 16.0 |
| | Hz, 1H), 7.63 (s, 1H), 7.58 (d, J = 7.5 Hz, 1H), 7.47 (s, 1H), 7.44 (t, J = 8.0 |
| | Hz, 1H), 7.35 (d, J = 7.9 Hz, 2H), 7.28 (d, J = 4.6 Hz, 1H), 7.17-7.12 (m, |
| | 2H), 7.00 (d, J = 8.0 Hz, 2H), 6.87 (d, J = 18.5 Hz, 1H), 6.69-6.27 (m, 1H), |
| | 5.02-4.98 (m, 1H), 4.60-4.56 (m, 1H), 4.52 (s, 2H), 4.22-4.14 (m, 2H), |
| | 3.99-3.94 (m, 1H), 3.85 (d, J = 10.4 Hz, 1H), 2.50-2.42 (m, 1H), 2.28-2.20 |
| | (m, 1H). LC/MS (ESI) m/z: 564 (M + H)⁺. RT (Method A): 1.35 min. |
| 271 | ¹H NMR (400 MHz, CD₃OD): δ 8.82 (d, J = 10.4 Hz, 1H), 8.22 (s, 1H), 7.94 |
| | (s, 1H), 7.42 (d, J = 15.7 Hz, 2H), 7.30 (d, J = 18.3 Hz, 3H), 7.23-7.14 (m, |
| | 3H), 7.06 (d, J = 21.4 Hz, 2H), 4.66 (s, 1H), 4.62 (d, 1H), 4.55 (d, 1H), 4.35 |
| | (d, 1H), 4.29 (d, 1H), 3.43 (s, 1H), 2.53-2.43 (m, 1H), 2.26-2.16 (m, 1H), |
| | 1.32 (s, 3H), 1.25-1.18 (m, 1H), 0.89-0.84 (m, 1H). LC/MS (ESI) m/z: 554 |
| | (M + H)⁺. RT (Method A): 1.46 min. |
| 277 | ¹H NMR (400 MHz, CD₃OD): δ 8.76 (s, 1H), 8.16 (d, J = 5.3 Hz, 1H), 7.81 (s, |
| | 1H), 7.54 (d, J = 7.6 Hz, 1H), 7.42-7.31 (m, 5H), 7.16-7.07 (m, 3H), 6.99 |
| | (d, J = 7.8 Hz, 2H), 4.83 (s, 1H), 4.51 (s, 2H), 4.32 (s, 2H), 3.43-3.36 (m, |
| | 1H), 2.42 (t, J = 13.0 Hz, 1H), 2.24-2.10 (m, 1H), 1.29 (s, 3H), 1.20-1.15 |
| | (m, 1H), 0.83-0.76 (m, 1H). LC/MS (ESI) m/z: 524 (M + H)⁺. RT (Method |
| | A): 1.37 min. |
| 278 | ¹H NMR (400 MHz, CD₃OD): δ 8.59 (s, 1H), 7.98 (d, J = 4.7 Hz, 1H), 7.62 (s, |
| | 1H), 7.59-7.55 (m, 1H), 7.46-7.41 (m, 2H), 7.28 (d, J = 5.0 Hz, 1H), 7.17- |
| | 7.10 (m, 3H), 7.09-7.05 (m, 1H), 7.05-7.00 (m, 2H), 6.69-6.31 (m, 1H), |
| | 5.02-4.99 (m, 1H), 4.60-4.57 (m, 2H), 4.55-4.50 (m, 2H), 4.24-4.14 (m, |
| | 2H), 4.00-3.94 (m, 1H), 3.87-3.82 (m, 1H), 2.52-2.43 (m, 1H), 2.28- |
| | 2.19 (m, 1H). LC/MS (ESI) m/z: 582 (M + H)⁻. RT (Method A): 1.39 min. |
| 280 | LC/MS (ESI) m/z: 546 (M + H)⁺. RT (Method A): 1.43 min |
| 281 | LC/MS (ESI) m/z: 534 (M + H)⁺. RT (Method A): 1.56 min |
| 286 | LC/MS (ESI) m/z: 622 (M + H)⁺. RT (Method A): 1.68 min |
| 373^{b} | ¹H NMR (400 MHz, DMSO-d6) δ 8.66 (t, J = 5.6 Hz, 1H), 8.30 (t, J = 6.0 Hz, |
| | 1H), 7.87 (d, J = 8.7 Hz, 1H), 7.44 (t, J = 7.8 Hz, 1H), 7.38-7.27 (m, 1H), |
| | 7.22 (dd, J = 15.8, 7.9 Hz, 1H), 7.09 (d, J = 8.0 Hz, 1H), 7.06-6.99 (m, 1H), |
| | 4.86-4.55 (m, 1H), 4.42-4.24 (m, 1H), 4.22 (t, J = 5.1 Hz, 1H), 4.04 (dd, J = |
| | 16.4, 5.4 Hz, 1H), 3.51 (d, J = 6.5 Hz, 1H), 3.43 (dd, J = 6.1, 2.4 Hz, 1H), |
| | 2.28 (t, J = 12.4 Hz, 1H), 2.00 (dd, J = 13.3, 3.2 Hz, 1H), 1.23-1.98 (m, 3H), |
| | 1.18 (d, J = 6.7 Hz, 1H), 0.93 (s, 3H), 0.64-0.47 (m, 2H). LC/MS (ESI) m/z: |
| | 518 (M + H)⁺. RT (Method A): 2.52 min. |

^{a}PyBOP was used in place of HATU.
^{b}T₃P was used in place of HATU.

Compounds 359, 360, and 363-365 can be prepared based on Step 9 in Scheme 1 with the following reactants:

| Cmpd # | Reactant A | Reactant B |
|---|---|---|
| 359 | 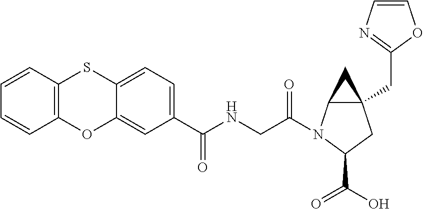 [SMILES: O=C(NCC(=O)N1[C@H]2C[C@@]2(Cc2ncco2)C[C@H]1C(=O)O)c1ccc2c(c1)Oc1ccccc1S2] | 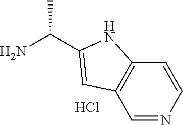 |
| 360 | 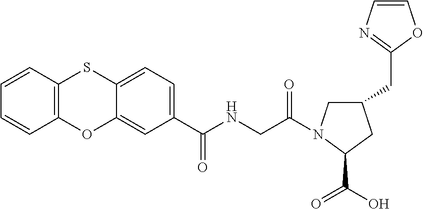 [SMILES: O=C(NCC(=O)N1C[C@H](Cc2ncco2)C[C@H]1C(=O)O)c1ccc2c(c1)Oc1ccccc1S2] | 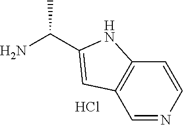 |
| 363 | 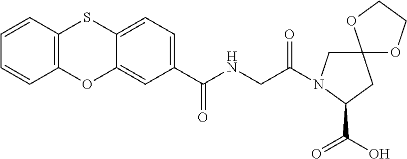 [SMILES: O=C(NCC(=O)N1CC2(C[C@H]1C(=O)O)OCCO2)c1ccc2c(c1)Oc1ccccc1S2] | 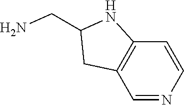 [SMILES: NCC1Cc2cnccc2N1] |
| 364 | 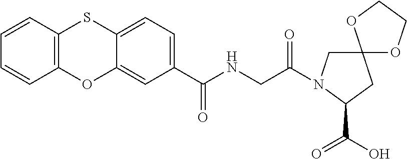 [SMILES: O=C(NCC(=O)N1CC2(C[C@H]1C(=O)O)OCCO2)c1ccc2c(c1)Oc1ccccc1S2] | 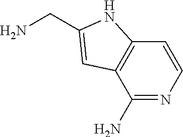 [SMILES: NCc1cc2c(N)nccc2[nH]1] |
| 365 | 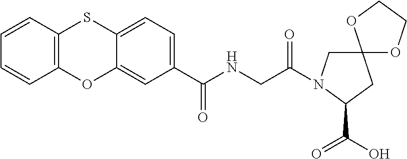 [SMILES: O=C(NCC(=O)N1CC2(C[C@H]1C(=O)O)OCCO2)c1ccc2c(c1)Oc1ccccc1S2] | 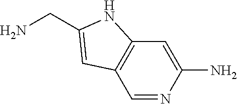 [SMILES: NCc1cc2cnc(N)cc2[nH]1] |

### Scheme 2. Synthesis of (S)-N-((1H-pyrrolo[3,2-c]pyridin-2-yl)methyl)-7-((phenoxathiine-3-carbonyl)glycyl)-1,4-dioxa-7-azaspiro[4.4]nonane-8-carboxamide (Compound 4)

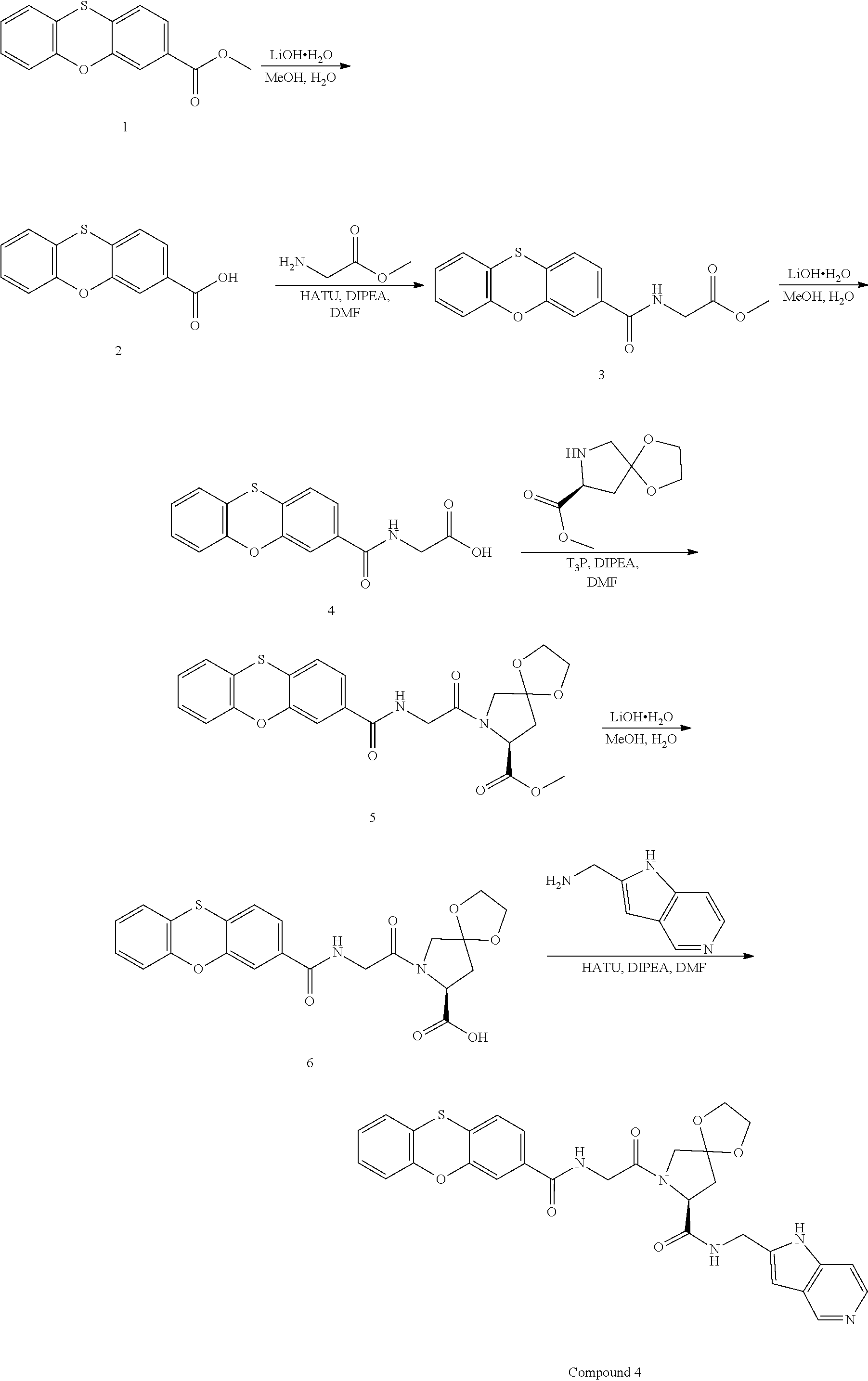

### Step 1: phenoxathiine-3-carboxylic acid (2)

To a solution of methyl phenoxathiine-2-carboxylate (400 mg, 1.55 mmol) in MeOH (5 mL)/THF (1 mL)/H₂O (2 mL) was added LiOH·H₂O (195.3 mg, 4.65 mmol) at 0° C. and the mixture was stirred at room temperature for 2 hours. The mixture was acidified with IN aq. HCl to pH-3 and extracted with EtOAc twice. The combined organic layers were washed with brine, dried over anhydrous Na₂SO₄, filtered, and concentrated under reduced pressure to dryness to give phenoxathiine-3-carboxylic acid (370 mg, yield 97.8%) as a yellow solid, which was used directly in the next step without purification. LC/MS (ESI) (m/z): 245 (M+H)⁺.

### Step 2: methyl (phenoxathine-3-carbonyl)glycinate (3)

To a mixture of phenoxathiine-3-carboxylic acid (370 mg, 1.52 mmol) and methyl glycinate (380.6 mg, 3.04 mmol) in DMF (5 mL) was added DIPEA (1.17 g, 9.12 mmol) and HATU (691.5 mg, 1.82 mmol) at 0° C. under N₂ atmosphere and the mixture was stirred at 25° C. for 2 hours. The mixture was diluted with EtOAc and washed with water twice. The combined organic layers were dried over anhydrous Na₂SO₄, filtered, and concentrated under reduced pressure to dryness. The residue was purified by flash chromatography (silica gel, 0-33% EtOAc in PE) to give methyl (phenoxathiine-3-carbonyl)glycinate (428 mg, yield 89.6%) as a white solid. LC/MS (ESI) m/z: 316 (M+H)⁺.

### Step 3: (phenoxathine-3-carbonyl)glycine (4)

To a solution of methyl (phenoxathiine-3-carbonyl)glycinate (428 mg, 1.36 mmol) in MeOH (5 mL)/THF (1 mL)/H₂O (2 mL) was added LiOH·H₂O (171.2 mg, 2.72 mmol) at 0° C. and the mixture was stirred at room temperature for 1 hour. The mixture was acidified with IN aq. HCl to pH-3 then extracted with EtOAc twice. The combined organic layers were washed with brine, dried over anhydrous Na₂SO₄, filtered, and concentrated under reduced pressure to dryness to give (phenoxathiine-3-carbonyl)glycine (350 mg, yield 85.6%) as a white solid, which was used directly in the next step. LC/MS (ESI) (m/z): 302 (M+H)⁺.

### Step 4: methyl (S)-7-((phenoxathiine-3-carbonyl)glycyl)-1,4-dioxa-7-azaspiro[4.4]nonane-8-carboxylate (5)

To a mixture of (phenoxathiine-3-carbonyl)glycine (100 mg, 0.33 mmol) and methyl (S)-1,4-dioxa-7-azaspiro[4.4]nonane-8-carboxylate (93.1 mg, 0.49 mmol) in DMF (3.0 mL) was added DIPEA (257 mg, 1.98 mmol) and T3P (633.9 mg, 0.99 mmol, 50% wt. in EtOAc) at 0° C. under N₂ atmosphere and the mixture was stirred at 25° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-69% EtOAc in PE) to give methyl (S)-7-((phenoxathiine-3-carbonyl)glycyl)-1,4-dioxa-7-azaspiro[4.4]nonane-8-carboxylate (135 mg, yield 86.4%) as a white solid. LC/MS (ESI) m/z: 471 (M+H)⁺.

### Step 5: (S)-7-((phenoxathiine-3-carbonyl)glycyl)-1,4-dioxa-7-azaspiro[4.4]nonane-8-carboxylic acid (6)

To a solution of methyl (S)-7-((phenoxathiine-3-carbonyl)glycyl)-1,4-dioxa-7-azaspiro [4.4]nonane-8-carboxylate (135 mg, 0.29 mmol) in MeOH (3 mL) and H₂O (1 mL) was added LiOH·H₂O (36.2 mg, 0.87 nmol) at 0° C. and the mixture was stirred at room temperature for 1 hour. The mixture was acidified with 1N aq. HCl to pH-3 and extracted with EtOAc twice. The combined organic layers were washed with brine, dried over anhydrous Na₂SO₄, filtered, and concentrated under reduced pressure to dryness to give (S)-7-((phenoxathiine-3-carbonyl)glycyl)-1,4-dioxa-7-azaspiro[4.4]nonane-8-carboxylic acid (110 mg, yield 83.9%) as a white solid, which was used directly in the next step. LC/MS (ESI) (m/z): 457 (M+H)⁻.

### Step 6: (S)-N-((1H-pyrrolo[3,2-c]pyridin-2-yl)methyl)-7-((phenoxathiine-3-carbonyl) glycyl)-1,4-dioxa-7-azaspiro[4.4]nonane-8-carboxamide (Compound 4)

To a mixture of (S)-7-((phenoxathiine-3-carbonyl)glycyl)-1,4-dioxa-7-azaspiro[4.4]nonane-8-carboxylic acid (60 mg, 0.13 mmol) and (1H-pyrrolo[3,2-c]pyridin-2-yl)methanamine (23.2 mg, 0.16 mmol) in DMF (3 mL) was added DIPEA (101.8 mg, 0.78 mmol) and HATU (60 mg, 0.16 mmol) at 0° C. and the mixture was stirred under N₂ atmosphere at room temperature for 1 hour. The mixture was concentrated under reduced pressure to dryness. The residue was purified by prep-TLC (DCM:MeOH=14:1) and further purified by prep-HPLC to give Compound 4 (15 mg, yield 19.5%) as a white solid. ¹H NMR (400 MHz, CD₃OD): δ 8.75 (s, 1H), 8.48 (s, 1H), 8.05 (d, J=6.4 Hz, 1H), 7.50 (d, J=6.4 Hz, 1H), 7.47-7.44 (m, 1H), 7.34 (d, J=1.8 Hz, 1H), 7.20 (dd, J=4.0, 3.2 Hz, 1H), 7.16-7.13 (m, 2H), 7.11-7.08 (m, 1H), 7.03-7.00 (m, 1H), 6.76 (s, 1H), 4.71 (d, J=16.6 Hz, 1H), 4.63 (dd, J=10.2, 7.2 Hz, 2H), 4.22 (d, J=16.4 Hz, 1H), 4.09 (d, J=16.4 Hz, 1H), 4.02 (d, J=3.5 Hz, 4H), 3.88 (d, J=10.8 Hz, 1H), 3.82 (d, J=10.7 Hz, 1H), 2.51-2.45 (m, 1H), 2.31-2.25 (m, 1H). LC/MS (ESI) (m/z): 586 (M+H)⁺. RT (Method A): 1.15 min.

The following compounds were prepared based on Scheme 2:

| Cmpd # | Reactant A | Reactant B | Reactant C | Reactant D | Characterization Data |
|---|---|---|---|---|---|
| 297 | 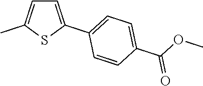 | 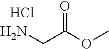 | 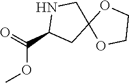 | 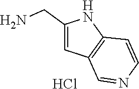 [SMILES: NCc1cc2cnccc2[nH]1 ; Cl] | ¹H NMR (400 MHz, CD₃OD): δ 8.82 (s, 1H), 8.05 (d, J = 6.5 Hz, 1H), 7.76 (d, J = 8.4 Hz, 2H), 7.58 (d, J = 8.5 Hz, 3H), 7.30 (d, J = 3.5 Hz, 1H), 6.84- 6.80 (m, 2H), 4.71-4.66 (m, 2H), 4.65-4.61 (m, 1H), 4.25 (d, J = 16.5 Hz, 1H), 4.11 (d, J = 16.6 Hz, 1H), 4.04- 4.01 (m, 4H), 3.90- 3.81 (m, 2H), 2.52 (s, 3H), 2.50- 2.42 (m, 1H), 2.31-2.25 (m, 1H). LC/MS (ESI) m/z: 560 (M + H)⁺. RT (Method A): 1.30 min. |
| 319^{a} | 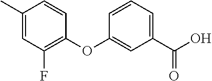 | 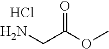 | 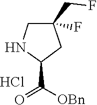 [SMILES: Cl ; O=C(OCc1ccccc1)[C@@H]1C[C@](F)(CF)CN1] | 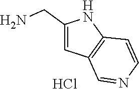 [SMILES: NCc1cc2cnccc2[nH]1 ; Cl] | ¹H NMR (400 MHz, CD₃OD): δ 8.79 (s, 1H), 8.49 (s, 1H), 8.05 (d, J = 6.3 Hz, 1H), 7.55 (d, J = 7.8 Hz, 1H), 7.45 (d, J = 6.4 Hz, 1H), 7.41 (d, J = 4.0 Hz, 2H), 7.12-7.07 (m, 2H), 7.05 (s, 1H), 7.00 (s, 1H), 6.74 (s, 1H), 4.77 (d, J = 6.3 Hz, 1H), 4.69 (m, 2H), 4.64 (s, 1H), 4.57 (d, J = 16.3 Hz, 1H), 4.26 (d, J = 16.7 Hz, 1H), 4.20 (s, 1H), 4.14-4.08 (m, 1H), 3.99 (d, J = 20.3 Hz, 1H), 2.67-2.57 (m, 1H), 2.34 (s, 3H), 2.30-2.17 (m, 1H). LCMS (ESI) (m/z): 580 (M + H)⁺. RT (Method A): 1.49 min. |

^{a}Steps 2-6 only.

### Scheme 3. Synthesis of (2S,4R)-N-((1H-pyrrolo[3,2-c]pyridin-2-yl)methyl)-1-((9,9-difluoro-9H-fluorene-3-carbonyl)glycyl)-4-fluoro-4-(methoxymethyl)pyrrolidine-2-carboxamide (Compound 5)

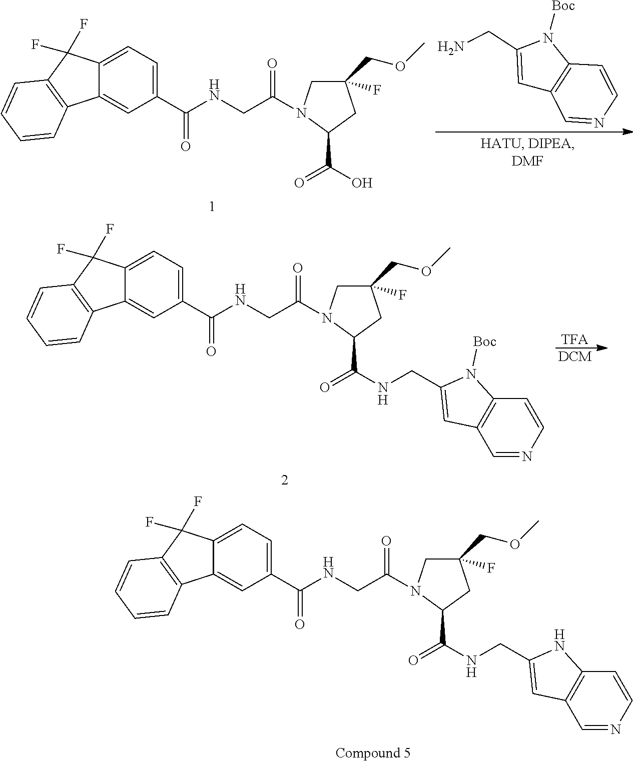

### Step 1: tert-butyl 2-(((2S,4R)-1-((9,9-difluoro-9H-fluorene-3-carbonyl)glycyl)-4-fluoro-4-(methoxymethyl)pyrrolidine-2-carboxamido)methyl)-1H-pyrrolo[3,2-c]pyridine-1-carboxylate

To a mixture of (2S,4R)-1-((9,9-difluoro-9H-fluorene-3-carbonyl)glycyl)-4-fluoro-4-(methoxymethyl)pyrrolidine-2-carboxylic acid (50 mg, 0.11 mmol) and tert-butyl 2-(aminomethyl)-1H-pyrrolo[3,2-c]pyridine-1-carboxylate (55 mg, 0.22 mmol) in DMF (3 mL) was added DIPEA (75 mg, 0.58 mmol) and HATU (62 mg, 0.16 mmol) at 0° C. under N₂ atmosphere and the mixture was stirred at room temperature for 1 hour. The reaction mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% McOH in DCM) to give tert-butyl 2-(((2S,4R)-1-((9,9-difluoro-9H-fluorene-3-carbonyl)glycyl)-4-fluoro-4-(methoxymethyl) pyrrolidine-2-carboxamido)methyl)-1H-pyrrolo[3,2-c]pyridine-1-carboxylate (60 mg, yield 93.8%) as a light oil. LC/MS (ESI) (m/z): 692 (M+H)⁺.

### Step 2: (2S,4R)-N-((1H-pyrrolo[3,2-c]pyridin-2-yl)methyl)-1-((9,9-difluoro-9H-fluorene-3-carbonyl)glycyl)-4-fluoro-4-(methoxymethyl)pyrrolidine-2-carboxamide (Compound 5)

To a solution of tert-butyl 2-(((2S,4R)-1-((9,9-difluoro-9H-fluorene-3-carbonyl)glycyl)-4-fluoro-4-(methoxymethyl)pyrrolidine-2-carboxamido)methyl)-1H-pyrrolo[3,2-c]pyridine-1-carboxylate (60 mg, 0.087 mmol) in DCM (3 mL) was added TFA (3 mL) and the reaction mixture was stirred at room temperature for 4 hours. The mixture was concentrated to dryness under reduced pressure and further purified by prep-HPLC to give Compound 5 (10 mg, 19.5% yield) as a white solid. ¹H NMR (400 MHz, CD₃OD): δ 8.81 (d, J=53.6 Hz, 1H), 8.46 (s, 1H), 8.13 (d, J=20.8 Hz, 1H), 7.95 (d, J=6.3 Hz, 1H), 7.85 (dd, J=7.8, 1.4 Hz, 1H), 7.68 (dd, J=15.0, 7.7 Hz, 3H), 7.57 (t, J=7.5 Hz, 1H), 7.45 (dd, J=10.3, 6.9 Hz, 2H), 6.79 (d, J=23.9 Hz, 1H), 4.74 (d, J=16.4 Hz, 1H), 4.69-4.61 (m, 2H), 4.35-4.22 (m, 2H), 4.14-3.94 (m, 2H), 3.80-3.68 (m, 2H), 3.43 (d, J=16.9 Hz, 3H), 2.65-2.52 (m, 1H), 2.26 (ddd, J=37.4, 14.2, 9.8 Hz, 1H). LC/MS (ESI) (m/z): 592 (M+H)⁺. RT (Method A): 1.20 min.

The following compounds were prepared based on Scheme 3:

| Cmpd | | |
|---|---|---|
| # | Reactant A | Reactant B |
| 6 | 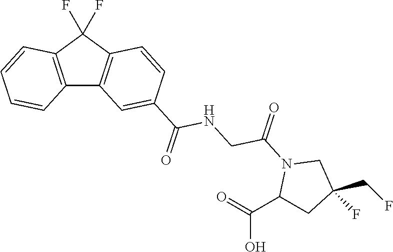 [SMILES: O=C(NCC(=O)N1C[C@@](F)(CF)CC1C(=O)O)c1ccc2c(c1)-c1ccccc1C2(F)F] | 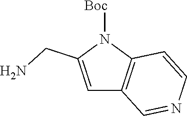 [SMILES: CC(C)(C)OC(=O)n1c(CN)cc2cnccc21] |
| 313^{a} | 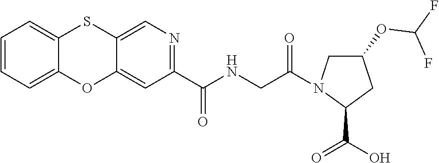 [SMILES: O=C(NCC(=O)N1C[C@H](OC(F)F)C[C@H]1C(=O)O)c1cc2c(cn1)Sc1ccccc1O2] | 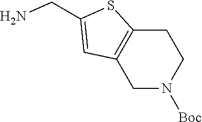 [SMILES: CC(C)(C)OC(=O)N1CCc2sc(CN)cc2C1] |
| 339 | 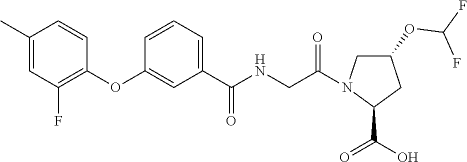 [SMILES: Cc1ccc(Oc2cccc(C(=O)NCC(=O)N3C[C@H](OC(F)F)C[C@H]3C(=O)O)c2)c(F)c1] | 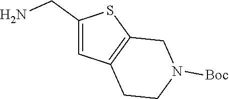 [SMILES: CC(C)(C)OC(=O)N1CCc2cc(CN)sc2C1] |

| Cmpd | |
|---|---|
| # | Characterization Data |
| 6 | ¹H NMR (400 MHz, CD₃OD): δ 8.83 (s, 1H), 8.08 (d, J = 3.6 Hz, 1H), 8.02 (dd, J = |
| | 6.7, 0.7 Hz, 1H), 7.84 (dd, J = 7.8, 1.5 Hz, 1H), 7.68 (t, J = 8.0 Hz, 3H), 7.62 (d, J = |
| | 6.7 Hz, 1H), 7.57 (t, J = 7.5 Hz, 1H), 7.46 (t, J = 7.1 Hz, 1H), 6.91 (d, J = 0.7 Hz, |
| | 1H), 4.79 (d, J = 1.8 Hz, 1H), 4.74 (s, 1H), 4.69 (t, J = 13.5 Hz, 3H), 4.28 (s, 2H), |
| | 4.20-4.01 (m, 2H), 2.73-2.61 (m, 1H), 2.30 (ddd, J = 35.8, 14.2, 9.5 Hz, 1H). |
| | LC/MS (ESI) (m/z): 580 (M + H)⁺. RT (Method A): 1.26 min. |
| 313^{a} | ¹H NMR (400 MHz, CD₃OD): δ 8.51 (s, 1H), 7.58-7.52 (m, 1H), 7.50 (d, J = 1.7 |
| | Hz, 1H), 7.26-7.14 (m, 3H), 7.10-7.03 (m, 2H), 6.83-6.27 (m, 2H), 5.10-4.91 |
| | (m, 1H), 4.60-4.42 (m, 3H), 4.21-4.03 (m, 4H), 3.99-3.83 (m, 2H), 3.43-3.36 |
| | (m, 2H), 3.04-2.95 (m, 2H), 2.66-2.41 (m, 1H), 2.36-2.17 (m, 1H). LC/MS |
| | (ESI) m/z: 615 (M + H)⁺. RT (Method A): 1.08 min. |
| 339 | ¹H NMR (400 MHz, CD₃OD): δ 8.53 (s, 1H), 7.57 (d, J = 7.8 Hz, 1H), 7.44-7.40 |
| | (m, 2H), 7.11-7.07 (m, 2H), 7.03 (m, 2H), 6.76 (d, J = 14.3 Hz, 1H), 6.68-6.29 |
| | (m, 1H), 4.98 (d, J = 3.4 Hz, 1H), 4.52 (d, J = 7.8 Hz, 1H), 4.47 (d, J = 5.8 Hz, 2H), |
| | 4.19-4.13 (m, 4H), 3.96-3.91 (m, 1H), 3.83 (d, J = 10.6 Hz, 1H), 3.27 (m, 2H), |
| | 2.76 (m, 2H), 2.46-2.39 (m, 1H), 2.36 (s, 3H), 2.24-2.16 (m, 1H). LC/MS (ESI) |
| | m/z: 617 (M + H)⁺. |

^{a}HCl/1,4-dioxane was used in place of TFA in Step 2.

Compounds 340-345, 348-351, 361, and 362 can be prepared based on Scheme 3 with the following reactants:

| Cmpd # | Reactant A | Reactant B |
|---|---|---|
| 340 | 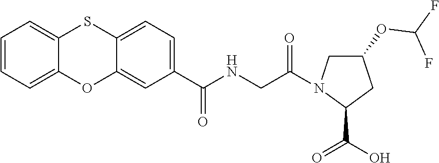 [SMILES: O=C(NCC(=O)N1C[C@H](OC(F)F)C[C@H]1C(=O)O)c1ccc2c(c1)Oc1ccccc1S2] | 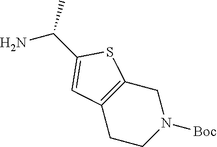 [SMILES: C[C@@H](N)c1cc2c(s1)CN(C(=O)OC(C)(C)C)CC2] |
| 341 | 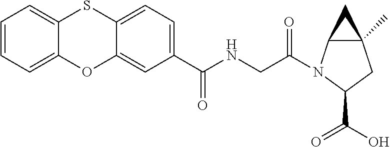 [SMILES: C[C@@]12C[C@@H]1N(C(=O)CNC(=O)c1ccc3c(c1)Oc1ccccc1S3)[C@H](C(=O)O)C2] | 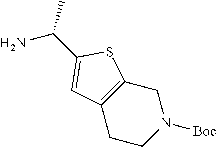 [SMILES: C[C@@H](N)c1cc2c(s1)CN(C(=O)OC(C)(C)C)CC2] |
| 342 | 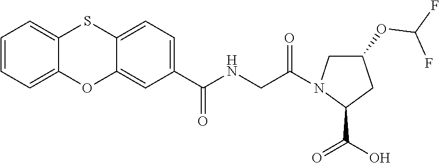 [SMILES: O=C(NCC(=O)N1C[C@H](OC(F)F)C[C@H]1C(=O)O)c1ccc2c(c1)Oc1ccccc1S2] | 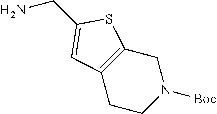 [SMILES: CC(C)(C)OC(=O)N1CCc2cc(CN)sc2C1] |
| 343 | 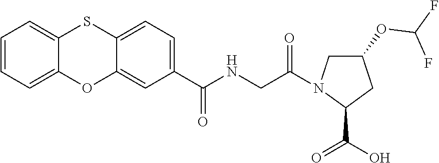 [SMILES: O=C(NCC(=O)N1C[C@H](OC(F)F)C[C@H]1C(=O)O)c1ccc2c(c1)Oc1ccccc1S2] | 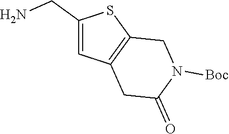 [SMILES: CC(C)(C)OC(=O)N1Cc2sc(CN)cc2CC1=O] |
| 344 | 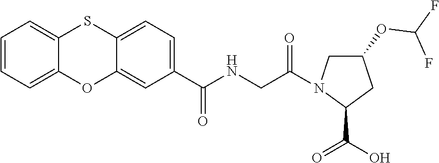 [SMILES: O=C(NCC(=O)N1C[C@H](OC(F)F)C[C@H]1C(=O)O)c1ccc2c(c1)Oc1ccccc1S2] | 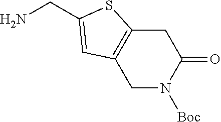 [SMILES: CC(C)(C)OC(=O)N1Cc2cc(CN)sc2CC1=O] |
| 345 | 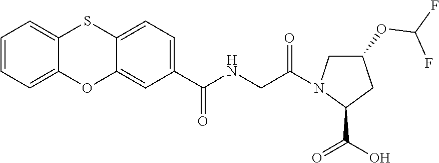 [SMILES: O=C(NCC(=O)N1C[C@H](OC(F)F)C[C@H]1C(=O)O)c1ccc2c(c1)Oc1ccccc1S2] | 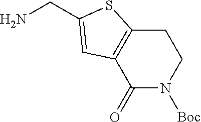 [SMILES: CC(C)(C)OC(=O)N1CCc2sc(CN)cc2C1=O] |
| 348 | 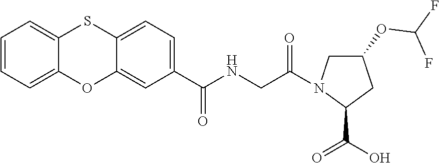 [SMILES: O=C(NCC(=O)N1C[C@H](OC(F)F)C[C@H]1C(=O)O)c1ccc2c(c1)Oc1ccccc1S2] | 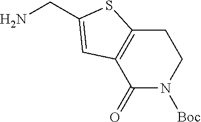 [SMILES: CC(C)(C)OC(=O)N1CCc2sc(CN)cc2C1=O] |
| 349 | 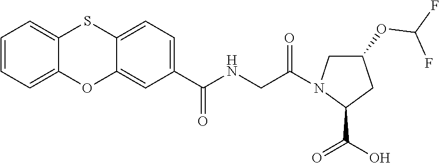 [SMILES: O=C(NCC(=O)N1C[C@H](OC(F)F)C[C@H]1C(=O)O)c1ccc2c(c1)Oc1ccccc1S2] | 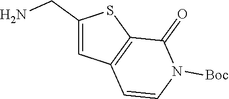 [SMILES: CC(C)(C)OC(=O)n1ccc2cc(CN)sc2c1=O] |
| 350 | 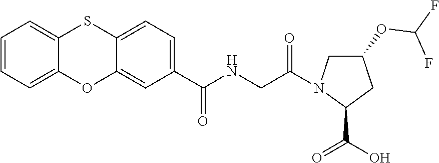 [SMILES: O=C(NCC(=O)N1C[C@H](OC(F)F)C[C@H]1C(=O)O)c1ccc2c(c1)Oc1ccccc1S2] | 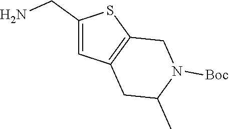 [SMILES: CC1Cc2cc(CN)sc2CN1C(=O)OC(C)(C)C] |
| 351 | 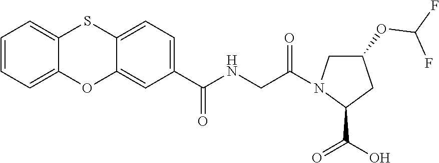 [SMILES: O=C(NCC(=O)N1C[C@H](OC(F)F)C[C@H]1C(=O)O)c1ccc2c(c1)Oc1ccccc1S2] | 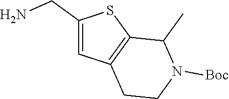 [SMILES: CC1c2sc(CN)cc2CCN1C(=O)OC(C)(C)C] |
| 361 | 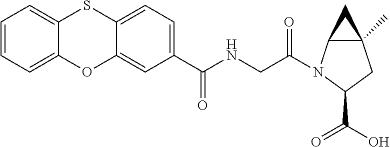 [SMILES: C[C@@]12C[C@@H]1N(C(=O)CNC(=O)c1ccc3c(c1)Oc1ccccc1S3)[C@H](C(=O)O)C2] | 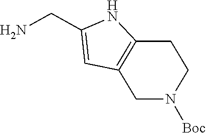 [SMILES: CC(C)(C)OC(=O)N1CCc2[nH]c(CN)cc2C1] |
| 362 | 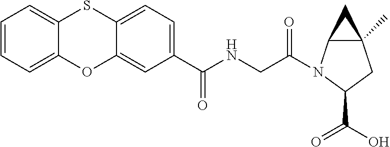 [SMILES: C[C@@]12C[C@@H]1N(C(=O)CNC(=O)c1ccc3c(c1)Oc1ccccc1S3)[C@H](C(=O)O)C2] | 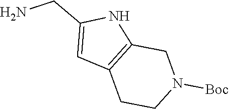 [SMILES: CC(C)(C)OC(=O)N1CCc2cc(CN)[nH]c2C1] |

### Scheme 4: (S)-N-((1H-pyrrolo[2,3-c]pyridin-2-yl)methyl)-7-((9,9-difluoro-9H-fluorene-3-carbonyl)glycyl)-1,4-dioxa-7-azaspiro[4.4]nonane-8-carboxamide (Compound 9)

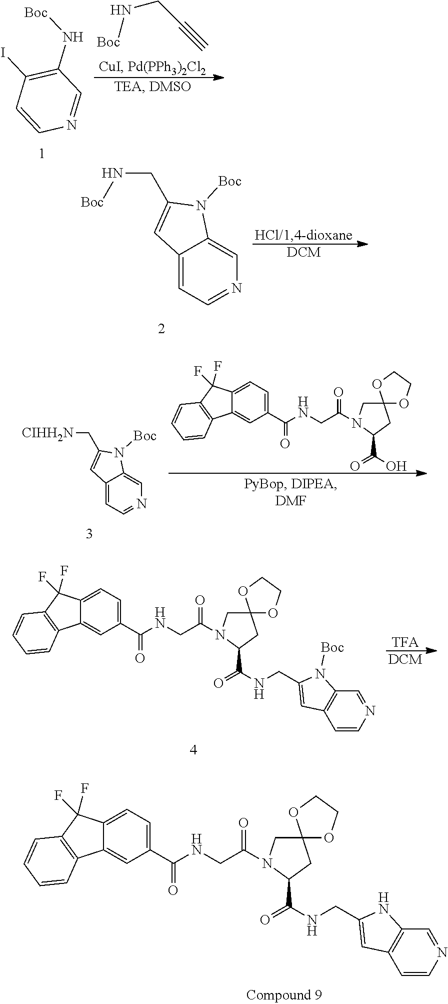

### Step 1: tert-butyl 2-(((tert-butoxycarbonyl)amino)methyl)-1H-pyrrolo[2,3-c]pyridine-1-carboxylate (2)

To a mixture of tert-butyl (4-iodopyridin-3-yl)carbamate (2.0 g, 6.25 mmol) and tert-butyl prop-2-yn-1-ylcarbamate (1.16 g, 7.5 mmol) in TEA (10 mL) and DMSO (5 mL) was added CuI (59.97 mg, 0.31 mmol) and Pd(PPh₃)₂Cl₂ (218.9 mg, 0.31 mmol) under N₂ atmosphere and the reaction mixture was stirred under N₂ atmosphere at 50° C. for 16 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give tert-butyl 2-(((tert-butoxycarbonyl)amino)methyl)-1H-pyrrolo[2,3-c]pyridine-1-carboxylate (550 mg, yield 25.4%) as a yellow solid. LC/MS (ESI) m/z: 348 (M+H)⁺.

### Step 2: tert-butyl 2-(aminomethyl)-H-pyrrolo[2,3-c]pyridine-1-carboxylate hydrochloride (3)

To a solution of tert-butyl 2-(((tert-butoxycarbonyl)amino)methyl)-1H-pyrrolo[2,3-c]pyridine-1-carboxylate (100 mg, 0.29 mmol) in DCM (1 mil) and HCl/1,4-dioxane (2 mL, 4M) and the mixture was stirred at room temperature for 2 hours. The reaction mixture was concentrated under reduced pressure to dryness to give tert-butyl 2-(aminomethyl)-1H-pyrrolo[2,3-c]pyridine-1-carboxylate hydrochloride (71 mg, yield 99.7%) as a brown solid, which was used directly in the next reaction without further purification. LC/MS (ESI) m/z: 248 (M+H)⁺.

### Step 3: tert-butyl (S)-2-((7-((9,9-difluoro-9H-fluorene-3-carbonyl)glycyl)-1,4-dioxa-7-azaspiro[4.4]nonane-8-carboxamido)methyl)-1H-pyrrolo[2,3-c]pyridine-1-carboxylate (4)

To a mixture of (S)-7-((9,9-difluoro-9H-fluorene-3-carbonyl)glycyl)-1,4-dioxa-7-azaspiro[4.4]nonane-8-carboxylic acid (70 mg, 0.15 mmol) and tert-butyl 2-(aminomethyl)-1H-pyrrolo[2,3-c]pyridine-1-carboxylate hydrochloride (56.6 mg, 0.23 mmol) in DMF (3 mL) was added DIPEA (118.3 mg, 0.9 mmol) and PyBOP (79.5 mg, 0.15 mmol) at 0° C. under N₂ atmosphere and the mixture was stirred at room temperature for 2 hours. The mixture was concentrated under reduced pressure to dryness. The residue was purified by prep-TLC (DCM:MeOH=10:1) to give tert-butyl (S)-2-((7-((9,9-difluoro-9H-fluorene-3-carbonyl)glycyl)-1,4-dioxa-7-azaspiro[4.4]nonane-8-carboxamido)methyl)-1H-pyrrolo[2,3-c]pyridine-1-carboxylate (70 mg, yield 54.9%) as a brown solid. LC/MS (ESI) m/z: 688 (M+H)⁺.

### Step 4: (S)-N-((1H-pyrrolo[2,3-c]pyridin-2-yl)methyl)-7-((9,9-difluoro-9H-fluorene-3-carbonyl)glycyl)-1,4-dioxa-7-azaspiro[4.4]nonane-8-carboxamide (Compound 9)

To a solution of ten-butyl (S)-2-((7-((9,9-difluoro-9H-fluorene-3-carbonyl)glycyl)-1,4-dioxa-7-azaspiro[4.4]nonane-8-carboxamido)methyl)-1H-pyrrolo[2,3-c]pyridine-1-carboxylate (50 mg, 0.07 mmol) in TFA (3 mL) and DCM (1 ml) and the mixture was stirred at room temperature for 2 hours. The reaction mixture was concentrated under reduced pressure to dryness. The residue was purified by prep-HPLC to give Compound 9 (20 mg, yield 34.2%) as a white solid. ¹H NMR (400 MHz, CD₃OD): δ 8.62 (s, 1H), 7.99 (d, J=10.4 Hz, 1H), 7.91 (s, 1H), 7.78 (s, 1H), 7.67-7.56 (m, 5H), 7.46 (dd, J=7.1, 4.0 Hz, 1H), 6.70 (d, J=5.1 Hz, 1H), 4.77 (d, J=15.7 Hz, 1H), 4.69 (d, J=16.6 Hz, 2H), 4.30 (dd, J=16.4, 2.1 Hz, 1H), 4.13 (d, J=16.5 Hz, 1H), 4.04 (s, 4H), 3.92 (d, J=10.9 Hz. 1H), 3.86 (d, J=10.8 Hz, 1H). 2.51 (dd, J=13.1, 9.2 Hz, 1H), 2.29 (dd, J=13.2, 5.7 Hz, 1H). LC/MS (ESI) m/z: 588 (M+H)⁺.

### Scheme 5. Synthesis of (S)-N-((1H-pyrrolo[2,3-b]pyridin-2-yl)methyl)-7-((9,9-difluoro-9H-fluorene-3-carbonyl)glycyl)-1,4-dioxa-7-azaspiro[4.4]nonane-8-carboxamide (Compound 10)

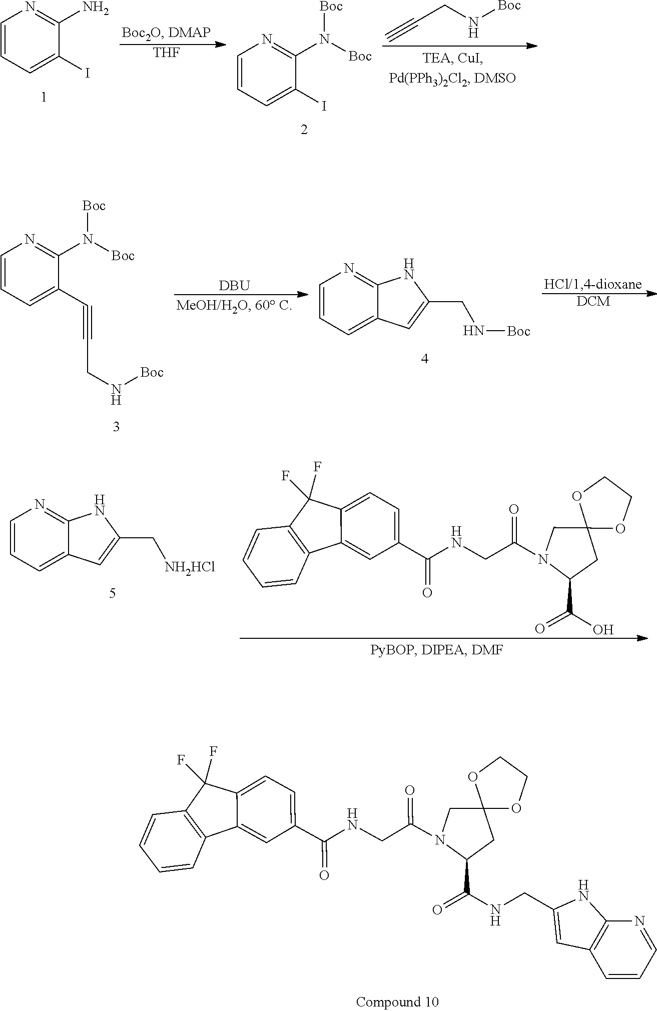

### Step 1: tert-butyl (3-iodopyridin-2-yl)(tert-butoxycarbonyl)carbamate

To a mixture of 3-iodopyridin-2-amine (4.17 g, 0.019 mol) and Boc₂O (8.26 g, 0.038 mol) in THF (40 mL) was added 4-DMAP (232 mg, 0.0019 mol) and the mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give tert-butyl (3-iodopyridin-2-yl)(tert-butoxycarbonyl) carbamate (7.4 g, yield 92.6%) as a white solid. LC/MS (ESI) m/z: 421 (M+H)⁺.

### Step 2: tert-butyl (tert-butoxycarbonyl)(3-(3-((tert-butoxycarbonyl)amino)prop-1-yn-1-yl)pyridin-2-yl)carbamate

To a mixture of tert-butyl (3-iodopyridin-2-yl)(tert-butoxycarbonyl)carbamate (2.0 g, 4.76 mmol) and tert-butyl prop-2-yn-1-ylcarbamate (886 mg, 5.71 mmol) in TEA (10 mL) and DMSO (5 mL) was added CuI (46 mg, 0.24 mmol) and Pd(PPh₃)₂Cl₂ (167 mg, 0.24 mmol) under N₂ atmosphere, the mixture was degassed under N₂ atmosphere for three times and stirred at 50° C. overnight.

The reaction mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give tert-butyl (tert-butoxycarbonyl)(3-(3-((tert-butoxycarbonyl)amino)prop-1-yn-1-yl)pyridin-2-yl) carbamate (1.5 g, yield 70.4%) as a brown oil. LC/MS (ESI) m/z: 448 (M+H)⁺.

### Step 3: tert-butyl ((1H-pyrrolo[2,3-b]pyridin-2-yl)methyl)carbamate

To a solution of tert-butyl (tert-butoxycarbonyl)(3-(3-((tert-butoxycarbonyl)amino) prop-1-yn-1-yl)pyridin-2-yl)carbamate (1.32 g, 2.95 mmol) in MeOH (6 mL)/H₂O (6 mL) was added DBU (899 mg, 5.91 mmol) and the reaction mixture was stirred at 60° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-15% MeOH in DCM) and further purified by prep-HPLC to give tert-butyl ((1H-pyrrolo[2,3-b]pyridin-2-yl)methyl)carbamate (150 mg, yield 20.1%) as a yellow oil. LC/MS (ESI) m/z: 248 (M+H)⁺.

### Step 4: (H-pyrrolo[2,3-b]pyridin-2-yl)methanamine hydrochloride

To a solution of tert-butyl ((1H-pyrrolo[2,3-b]pyridin-2-yl)methyl)carbamate (150 mg, 0.61 mmol) in DCM (2.0 mL) was added 1,4-dioxane (1.0 mL, 4M) and the reaction mixture was stirred at room temperature for 2 hours. The mixture was concentrated under reduced pressure to dryness to give (1H-pyrrolo[2,3-b]pyridin-2-yl)methanamine hydrochloride (150 mg, crude) as a yellow solid, which was directly used in next reaction without purification. LC/MS (ESI) m/z: 148 (M+H)⁺.

### Step 5: (S)-N-((1H-pyrrolo[2,3-b]pyridin-2-yl)methyl)-7-((9,9-difluoro-9H-fluorene-3-carbonyl)glycyl)-1,4-doxa-7-azaspiro[4.4]nonane-8-carboxamide (Compound 10)

To a mixture of (S)-7-((9,9-difluoro-9H-fluorene-3-carbonyl)glycyl)-1,4-dioxa-7-azaspiro[4.4]nonane-8-carboxylic acid (80 mg, 0.18 mmol) and (1H-pyrrolo[2,3-b]pyridin-2-yl)methanamine hydrochloride (51 mg, 0.35 mmol) in DMF (2 mL) was added DIPEA (136 mg, 1.05 mmol) and PyBOP (119 mg, 0.23 mmol) at 0° C. under N₂ atmosphere and the mixture was stirred at room temperature for 2 hours. The reaction mixture was diluted with water and extracted with EtOAc twice. The combined organic layers were washed with brine, dried over anhydrous Na₂SO₄, filtered, and concentrated under reduced pressure to dryness. The residue was purified by prep-HPLC (DCM:MeOH=8:1) and further purified by prep-HPLC to give Compound 10 (5.5 mg, yield 5.2%) as a white solid. ¹H NMR (400 MHz, CD₃OD): δ 8.13 (d, J=28.8 Hz, 1H), 7.98 (t, J=22.5 Hz, 1H), 7.82 (td, J=15.5, 7.9 Hz, 2H), 7.66 (dd, J=16.7, 7.4 Hz, 3H), 7.55 (t, J=7.5 Hz, 1H), 7.44 (t, J=7.4 Hz, 1H), 7.06-6.93 (m, 1H), 6.41 (d, J=23.2 Hz, 1H), 4.69-4.57 (m, 3H), 4.29-4.17 (m, 2H), 3.99 (d, J=27.1 Hz, 4H), 3.88-3.74 (m, 2H), 2.47 (dd, J=13.1, 9.1 Hz, 1H), 2.29 (dd, J=13.1, 5.8 Hz, 1H). LC/MS (ESI) (m/z): 588 (M+H)⁺. RT (Method A): 1.97 min.

The following compounds were prepared based on Scheme 5:

| Cmpd | | | |
|---|---|---|---|
| # | Reactant A | Reactant B | Reactant C |
| 42 | 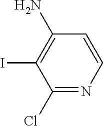 | 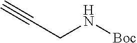 | 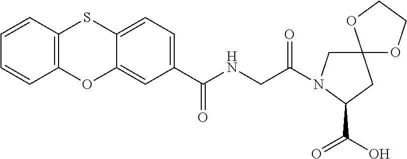 [SMILES: O=C(NCC(=O)N1CC2(C[C@H]1C(=O)O)OCCO2)c1ccc2c(c1)Oc1ccccc1S2] |
| 43 | 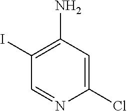 | 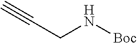 | 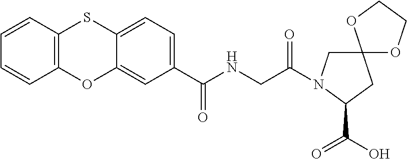 [SMILES: O=C(NCC(=O)N1CC2(C[C@H]1C(=O)O)OCCO2)c1ccc2c(c1)Oc1ccccc1S2] |
| 48 | 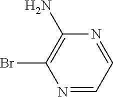 | 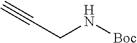 | 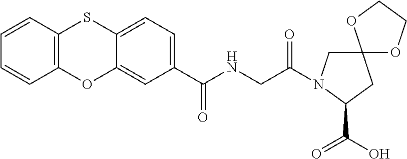 [SMILES: O=C(NCC(=O)N1CC2(C[C@H]1C(=O)O)OCCO2)c1ccc2c(c1)Oc1ccccc1S2] |
| 52 | 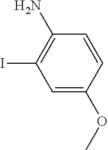 | 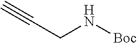 | 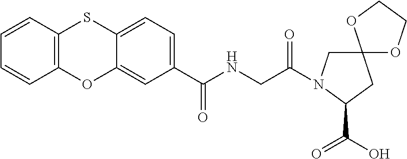 [SMILES: O=C(NCC(=O)N1CC2(C[C@H]1C(=O)O)OCCO2)c1ccc2c(c1)Oc1ccccc1S2] |
| 53 | 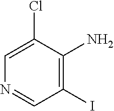 | 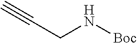 | 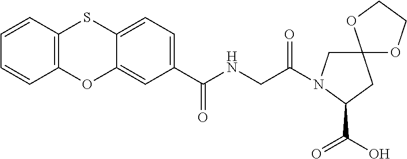 [SMILES: O=C(NCC(=O)N1CC2(C[C@H]1C(=O)O)OCCO2)c1ccc2c(c1)Oc1ccccc1S2] |

| Cmpd # | Characterization Data |
|---|---|
| 42 | ¹H NMR (400 MHz, DMSO-d6): δ 11.68 (d, J − 68.9 Hz, 1H), 8.78 |
| | (d, J = 4.5 Hz, 1H), 8.56 (t, J = 6.0 Hz, 1H), 7.85 (t, J = 17.3 Hz, |
| | 1H), 7.61-7.56 (m, 2H), 7.37 (d, J = 3.7 Hz, 1H), 7.29 (d, J = 7.7 |
| | Hz, 1H), 7.24 (t, J = 6.3 Hz, 2H), 7.12 (d, J = 9.0 Hz, 2H), 6.44 (s, |
| | 1H), 4.56-4.50 (m, 1H), 4.45-4.34 (m, 2H), 4.17 (dd, J = 16.7, |
| | 5.8 Hz, 1H), 4.03-3.94 (m, 5H), 3.80 (d, J = 11.1 Hz, 1H), 3.67 (d, |
| | J = 11.0 Hz, 1H), 2.33 (s, 1H), 2.13-2.07 (m, 1H). LC/MS (ESI) |
| | m/z: 620 (M + H)⁺. RT (Method A): 2.06 min. |
| 43 | ¹H NMR (400 MHz, CD₃OD): δ 8.39 (t, 1H), 7.52-7.45 (m, 1H), |
| | 7.39 (t, J = 12.0 Hz, 1H), 7.24-7.13 (m, 4H), 7.07 (dd, J = 11.2, |
| | 3.8 Hz, 1H), 7.02 (dd, J = 8.1, 1.1 Hz, 1H), 6.51 (t, J = 18.1 Hz, |
| | 1H), 4.64-4.56 (m, 3H), 4.15 (q, J = 16.5 Hz, 2H), 4.00 (dd, J = |
| | 11.3, 9.8 Hz, 4H), 3.83 (q, J = 10.7 Hz, 2H), 2.45 (dd, J = 13.1, 9.1 |
| | Hz, 1H), 2.27 (dd, J = 13.1, 6.2 Hz, 1H). LC/MS (ESI) m/z: 620 |
| | (M + H)⁺. RT (Method A): 2.04 min. |
| 48 | ¹H NMR (400 MHz, CD₃OD): δ 8.46 (s, 1H), 7.49 (dd, J = 19.7, 6.8 |
| | Hz, 3H), 7.44-7.27 (m, 1H), 7.22-7.10 (m, 3H), 7.08-6.99 (m, |
| | 2H), 6.92-6.72 (m, 1H), 6.31-6.08 (m, 1H), 4.81 (s, 1H), 4.22 |
| | (dd, J = 24.0, 9.8 Hz, 2H), 4.17-3.90 (m, 5H), 3.82 (q, J = 10.6 Hz, |
| | 2H), 2.71-2.49 (m, 1H), 2.39-2.17 (m, 1H). LC/MS (ESI) (m/z): |
| | 587 (M + H)⁺. RT (Method A): 1.43 min. |
| 52 | ¹H NMR (400 MHz, CD₃OD): δ 7.54-7.44 (m, 2H), 7.23-7.14 |
| | (m, 3H), 7.10-7.01 (m, 3H), 6.92 (dd, J = 28.2, 2.4 Hz, 1H), 6.64 |
| | (ddd, J = 37.2, 8.7, 2.2 Hz, 1H), 6.26 (d, J = 27.3 Hz, 1H), 4.62- |
| | 4.49 (m, 3H), 4.13 (d, J = 12.6 Hz, 2H), 4.02-3.93 (m, 4H), 3.79 (d, |
| | J = 2.2 Hz, 1H), 3.76-3.72 (m, 3H), 2.59-2.40 (m, 1H), 2.38-2.22 |
| | (m, 1H), 1.37 (d, J = 5.9 Hz, 1H). LC/MS (ESI) (m/z): 615 |
| | (M − H)⁺. RT (Method A): 2.33 min. |
| 53 | ¹H NMR (400 MHz, CD₃OD): δ 8.67 (d, J −55.9 Hz, 1H), 8.12 (s, |
| | 1H), 7.51-7.41 (m, 1H), 7.31 (d, J = 1.7 Hz, 1H), 7.20 (dd, J = 10.8, |
| | 4.5 Hz, 1H), 7.15-7.06 (m, 3H), 7.00 (dd, J = 8.0, 0.9 Hz, 1H), 6.77 |
| | (d, J = 28.7 Hz, 1H), 4.72 (dd, J = 16.0, 5.3 Hz, 1H), 4.66-4.59 (m, |
| | 2H), 4.21 (d, J = 16.3 Hz, 1H), 4.15-4.04 (m, 1H), 4.03 (d, J = 7.4 |
| | Hz, 4H), 3.95-3.88 (m, 1H), 3.84-3.73 (m, 1H), 2.62-2.46 (m, |
| | 1H), 2.43-2.25 (m, 1H). LC/MS (ESI) (m/z): 620 (M + H)⁺. RT |
| | (Method A): 1.33 min. |

### Scheme 6. Synthesis of (S)-N-((7H-pyrrolo[2,3-d]pyrimidin-6-yl)methyl)-7-((9,9-difluoro-9H-fluorene-3-carbonyl)glycyl)-1,4-dioxa-7-azaspiro[4.4]nonane-8-carboxamide (Compound 16)

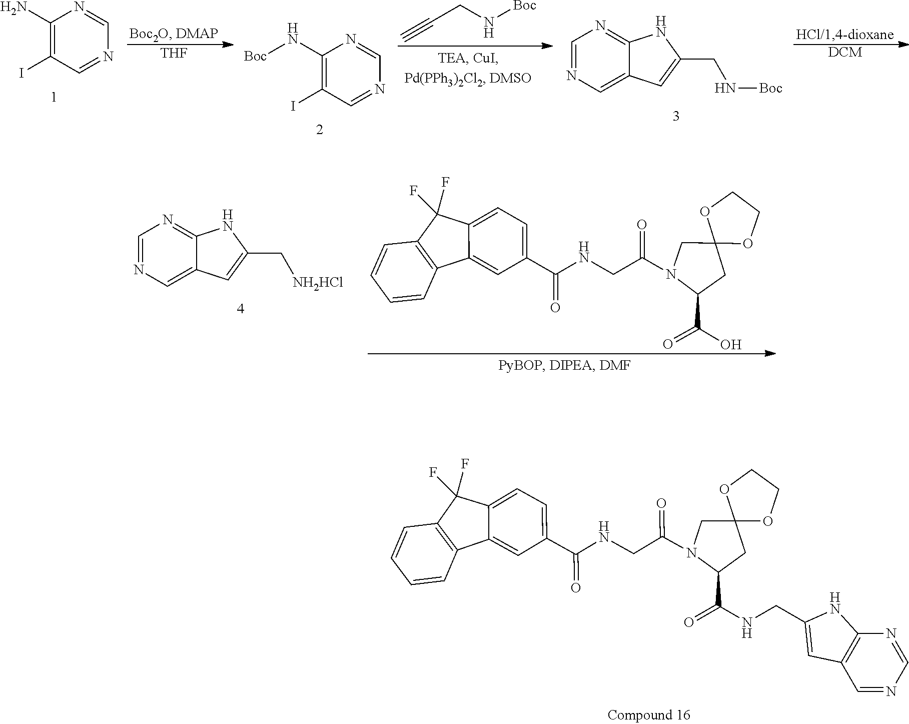

### Step 1: tert-butyl (5-iodopyrimidin-4-yl)carbamate

To a solution of 5-iodopyrimidin-4-amine (1.0 g, 4.52 mmol) in THF (10 mL) was added (Boc)₂O (1.97 g, 9.05 mmol) and DMAP (55 mg, 0.45 mmol) successively at 0° C. and the mixture was stirred at room temperature for 16 hours. The mixture was diluted with water and extracted with EtOAc twice. The combined organic layers were washed with brine, dried over anhydrous Na₂SO₄, filtered, and concentrated to dryness. The residue was purified by flash chromatography (silica gel, 0-50% EtOAc in PE) to give tert-butyl (5-iodopyrimidin-4-yl)carbamate (870 mg, yield 59.8%) as a white solid. LC/MS (ESI) m/z: 322 (M+H)⁺.

### Step 2: tert-butyl ((7H-pyrrolo[2,3-d]pyrimidin-6-yl)methyl)carbamate

To a mixture of tert-butyl (5-iodopyrimidin-4-yl)carbamate (400 mg, 1.25 mmol) and tert-butyl prop-2-yn-1-ylcarbamate (290 mg, 1.87 mmol) in TEA (3 mL) and DMSO (1.5 mL) was added CuI (12 mg, 0.063 mmol) and Pd(PPh₃)₂Cl₂ (44 mg, 0.063 mmol) under N₂ atmosphere, the mixture was degassed under N₂ atmosphere for three times and stirred at 50° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give tert-butyl ((7H-pyrrolo[2,3-d]pyrimidin-6-yl)methyl)carbamate (200 mg, yield 64.3%) as a brown oil. LC/MS (ESI) m/z: 249 (M+H)⁺.

### Step 3: (7H-pyrrolo[2,3-d]pyrimidin-6-yl)methanamine hydrochloride

A solution of tert-butyl ((7H-pyrrolo[2,3-d]pyrimidin-6-yl)methyl)carbamate (200 mg, 0.81 mmol) in HC/1,4-dioxane (2 mL, 4M) was stirred under N₂ atmosphere at room temperature for 2 hours. The reaction mixture was concentrated under reduced pressure to dryness to give (7H-pyrrolo[2,3-d]pyrimidin-6-yl)methanamine hydrochloride (110 mg, yield 91.7%) as a yellow solid, which was used directly in the next reaction without further purification. LC/MS (ESI) m/z: 149 (M+H)⁺.

### Step 4: (S)-N-((7H-pyrrolo[2,3-d]pyrimidin-6-yl)methyl)-7-((9,9-difluoro-9H-fluorene-3-carbonyl)glycyl)-1,4-dioxa-7-azaspiro[4.4]nonane-8-carboxamide (Compound 16)

To a mixture of (S)-7-((9,9-difluoro-9H-fluorene-3-carbonyl)glycyl)-1,4-dioxa-7-azaspiro[4.4]nonane-8-carboxylic acid (50 mg, 0.11 mmol) and (7H-pyrrolo[2,3-d]pyrimidin-6-yl)methanamine hydrochloride (26 mg, 0.14 mmol) in DMF (2 mL) was added DIPEA (85 mg, 0.66 mmol) and PyBOP (63 mg, 0.12 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=8:1) and further purified by prep-HPLC to give Compound 16 (10 mg, yield 15.4%) as a white solid. ¹H NMR (400 MHz, CD₃OD): δ 8.95 (d, J=60.6 Hz, 1H), 8.78 (d, J=49.1 Hz, 1H), 8.13 (d, J=32.8 Hz, 1H), 7.80 (dd, J=20.4, 7.5 Hz, 1H), 7.70 (d, J=7.4 Hz, 1H), 7.65 (d, J=7.8 Hz, 2H), 7.57 (t, J=7.6 Hz, 1H), 7.45 (t, J=7.3 Hz, 1H), 6.77 (d, J=29.2 Hz, 1H), 4.73-4.63 (m, 3H), 4.28 (d, J=16.5 Hz, 1H), 4.15 (dd, J=16.7, 8.5 Hz, 1H), 4.05-3.96 (m. 4H), 3.91-3.74 (m, 2H), 2.49 (dd, J=13.2, 9.1 Hz, 1H), 2.29 (dd, J=13.2, 5.6 Hz, 1H). LC/MS (ESI) m/z: 589 (M+H)⁺. RT (Method A): 1.52 min.

The following compounds were prepared based on Scheme 6:

| Cmpd # | Reactant A | Reactant B | Reactant C |
|---|---|---|---|
| 37 | 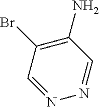 | 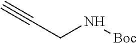 | 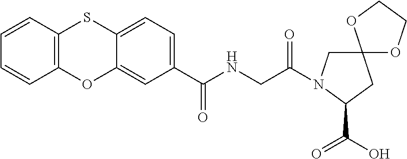 [SMILES: O=C(NCC(=O)N1CC2(C[C@H]1C(=O)O)OCCO2)c1ccc2c(c1)Oc1ccccc1S2] |
| 50^{a} | 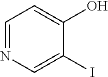 | 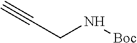 | 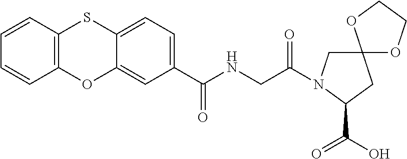 [SMILES: O=C(NCC(=O)N1CC2(C[C@H]1C(=O)O)OCCO2)c1ccc2c(c1)Oc1ccccc1S2] |

| Cmpd # | Characterization Data |
|---|---|
| 37 | ¹H NMR (400 MHz, CD₃OD): δ 9.26 (s, 1H), 9.13 (s, 1H), 8.19 (s, 1H), |
| | 7.45 (dd, J = 8.1, 1.8 Hz, 1H), 7.35 (d, J = 1.8 Hz, 1H), 7.23-7.18 (m, |
| | 1H), 7.15 (dd, J = 7.9, 1.6 Hz, 2H), 7.10-7.06 (m, 1H), 7.02 (dd, J = 8.1, |
| | 1.1 Hz, 1H), 6.83 (d, J = 24.4 Hz, 1H), 4.75 (t, J = 15.1 Hz, 2H), 4.64 (dd, |
| | J = 9.0, 5.9 Hz, 1H), 4.23 (d, J = 16.5 Hz, 1H), 4.10 (d, J = 16.4 Hz, 1H), |
| | 3.99 (d, J = 28.1 Hz, 4H), 3.85 (q, J = 10.8 Hz, 2H), 2.49 (dd, J = 13.1, 9.3 |
| | Hz, 1H), 2.28 (dd, J = 13.1, 5.8 Hz, 1H). LC/MS (ESI) m/z: 587 (M + H)⁺. |
| | RT (Method A): 1.31 min. |
| 50^{a} | ¹H NMR (400 MHz, CD₃OD): δ 8.85 (s, 1H), 8.42 (s, 1H), 7.73 (d, J = |
| | 6.1 Hz, 1H), 7.51 (dd, J = 8.1, 1.8 Hz, 1H), 7.42 (d, J = 1.8 Hz, 1H), 7.18 |
| | (ddd, J = 11.1, 9.4, 4.6 Hz, 3H), 7.11-6.98 (m, 3H), 4.65 (ddd, J = 27.2, |
| | 19.6, 12.4 Hz, 3H), 4.16 (dd, J = 48.1, 16.5 Hz, 2H), 4.01 (d, J = 4.8 Hz, |
| | 4H), 3.83 (q, J = 10.7 Hz, 2H), 2.48 (dd, J = 13.2, 9.3 Hz, 1H), 2.26 (dd, |
| | J = 13.2, 5.4 Hz, 1H). LC/MS (ESI) m/z: 587 (M + H)⁺. RT (Method A): |
| | 1.37 min. |

^{a}Steps 2-4 only.

### Scheme 7. Synthesis of (S)-N-((S)-1-(1H-pyrrolo[3,2-c]pyridin-2-yl)ethyl)-7-((9,9-difluoro-9H-fluorene-3-carbonyl)glycyl)-1,4-dioxa-7-azaspiro[4.4]nonane-8-carboxamide (Compound 23)

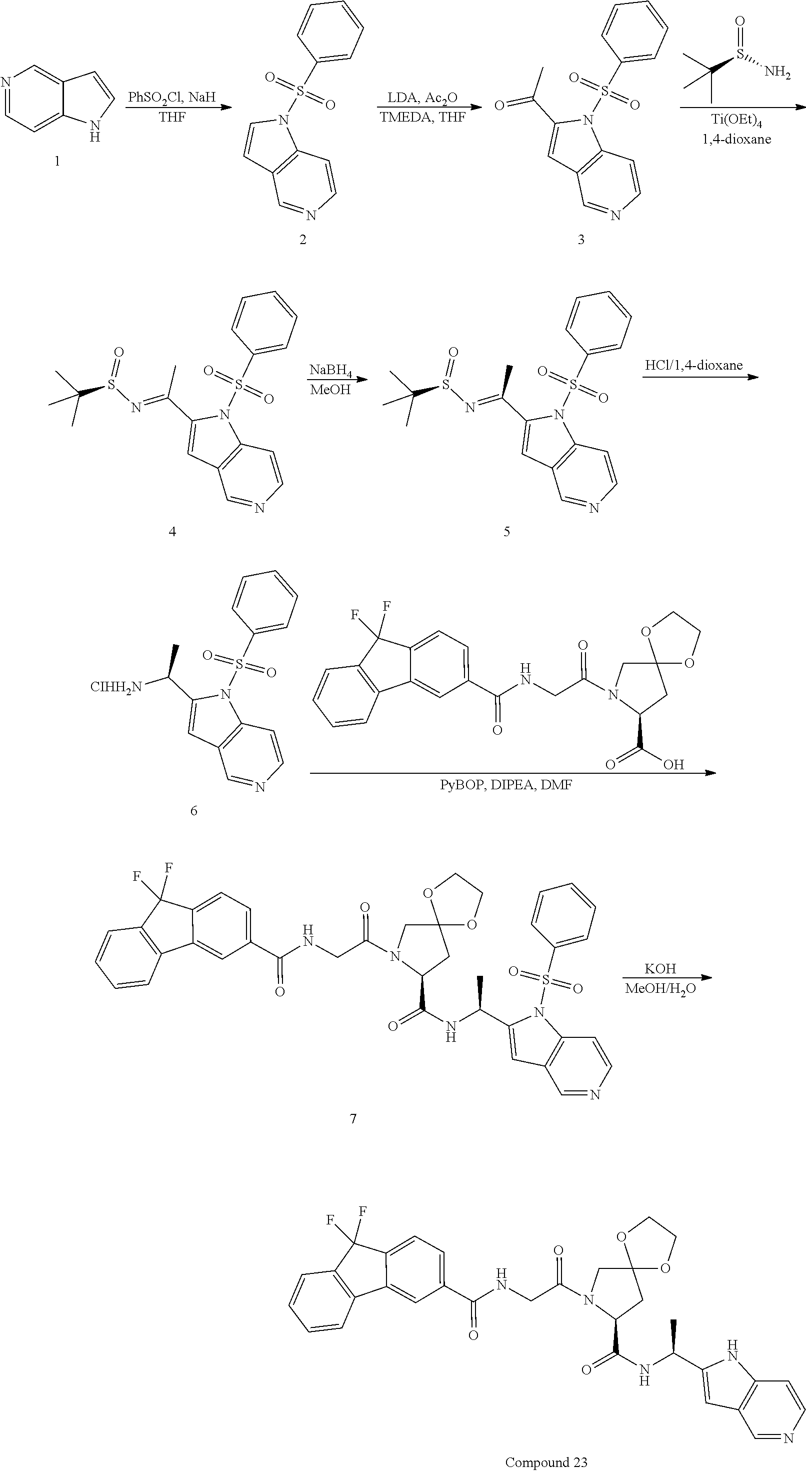

### Step 1: 1-(phenylsulfonyl)-H-pyrrolo[3,2-c]pyridine

To a solution of NaH (2.3 g, 97.5 mmol, 60% dispersion in mineral oil) in THE (200 mL) was added a solution of 1H-pyrrolo[3,2-c]pyridine (5 g, 42.4 mmol) in THE (50 mL) drop-wisely and the mixture was stirred at 0° C. for 1 hour. To the mixture was added a solution of PhSO₂Cl (8.2 g, 46.6 mmol) in THF (40 mL) at 0° C. and the resulting mixture was stirred at 28° C. for 2 hours. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel. 0-30% EtOAc in PE) to give 1-(phenylsulfonyl)-1H-pyrrolo[3,2-c]pyridine (9.0 g, yield 82.3%) as a light-yellow solid. LC/MS (ESI) m/z: 259 (M+H).

### Step 2: 1-(1-(phenylsulfonyl)-H-pyrrolo[3,2-c]pyridin-2-yl)ethan-1-one

To a mixture of 1-(phenylsulfonyl)-1H-pyrrolo[3,2-c]pyridine (2.0 g, 7.7 mmol) and TMEDA (895 mg, 7.7 mmol) in THF (50 mL) was added LDA (7.7 mL, 2 M in THF) drop-wisely at −70° C. and the reaction mixture was stirred at this temperature for 2 hours. To the mixture was added Ac₂O (1.2 g, 11.6 mmol) at −70° C. and the mixture was stirred at −70° C. to 0° C. for 2 hours. The mixture was quenched with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give 1-(1-(phenylsulfonyl)-1H-pyrrolo[3,2-c]pyridin-2-yl)ethan-1-one (700 mg, yield 30.3%) as a yellow solid. LC/MS (ESI) m/z: 301 (M+H)⁺.

### Step 3: (R,E)-2-methyl-N-(1-(1-(phenylsulfonyl)-H-pyrrolo[3,2-c]pyridin-2-yl) ethylidene)propane-2-sulfinamide

To a mixture of 1-(1-(phenylsulfonyl)-1H-pyrrolo[3,2-c]pyridin-2-yl)ethan-1-one (700 mg, 2.3 mmol) and (R)-2-methylpropane-2-sulfinamide (566 mg, 4.7 mmol) in 1,4-dioxane (10 mL) was added Ti(OEt)₄ (1.6 g, 6.9 mmol). The mixture was stirred at 120° C. for 4 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give (R,E)-2-methyl-N-(1-(1-(phenylsulfonyl)-1H-pyrrolo[3,2-c]pyridin-2-yl)ethyliden)propane-2-sulfinamide (320 mg, yield 34.5%) as a yellow solid. LC/MS (ESI) m/z: 404 (M+H)⁺.

### Step 4: (R)-2-methyl-N-((S)-1-(1-(phenylsulfonyl)-1H-pyrrolo[3,2-c]pyridin-2-yl)ethyl)propane-2-sulfinamide

To a solution of (R,E)-2-methyl-N-(1-(1-(phenylsulfonyl)-1H-pyrrolo[3,2-c]pyridin-2-yl)ethylidene)propane-2-sulfinamide (320 mg, 0.79 mmol) in MeOH (4 mL) was added NaBH₄ (45 mg. 1.2 mmol) in portions at 0° C. and the mixture was stirred at 20° C. for 10 minutes. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by prep-HPLC to give (R)-2-methyl-N-((S)-1-(1-(phenylsulfonyl)-1H-pyrrolo[3,2-c]pyridin-2-yl)ethyl)propane-2-sulfinamide (110 mg, yield 34.3%) as a yellow solid. LC/MS (ESI) m/z: 406 (M+H)⁺.

### Step 5: (S)-1-(1-(phenylsulfonyl)-1H-pyrrolo[3,2-c]pyridin-2-yl)ethan-1-amine hydrochloride

A solution of (R)-2-methyl-N-((S)-1-(1-(phenylsulfonyl)-1H-pyrrolo[3,2-c]pyridin-2-yl)ethyl)propane-2-sulfinamide (110 mg, 0.27 mmol) in HCl/1,4-dioxane (2 mL, 4M) was stirred under N₂ atmosphere at room temperature for 2 hours. The reaction mixture was concentrated under reduced pressure to dryness to give (S)-1-(1-(phenylsulfonyl)-1H-pyrrolo[3,2-c]pyridin-2-yl)ethan-1-amine hydrochloride (100 mg, yield 82.3%) as a light-yellow solid, which was used directly in the next step without further purification. LC/MS (ESI) m/z: 302 (M+H)⁺.

### Step 6: (S)-7-((9,9-difluoro-9H-fluorene-3-carbonyl)glycyl)-N-((S)-1-(-(phenylsulfonyl)-H-pyrrolo[3,2-c]pyridin-2-yl)ethyl)-1,4-dioxa-7-azaspiro[4.4]nonane-8-carboxamide

To a mixture of (S)-1-(1-(phenylsulfonyl)-1H-pyrrolo[3,2-c]pyridin-2-yl)ethan-1-amine hydrochloride (25 mg, 0.085 mmol) and (S)-7-((9,9-difluoro-9H-fluorene-3-carbonyl) glycyl)-1,4-dioxa-7-azaspiro[4.4]nonane-8-carboxylic acid (40 mg, 0.085 mmol) in DMF (0.7 mL) was added DIPEA (44 mg, 0.34 mmol) and PyBOP (44 mg, 0.085 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give (S)-7-((9,9-difluoro-9H-fluorene-3-carbonyl)glycyl)-N-((S)-1-(1-(phenylsulfonyl)-1H-pyrrolo[3,2-c]pyridin-2-yl)ethyl)-1,4-dioxa-7-azaspiro[4.4]nonane-8-carboxamide (10 mg, yield 15.9%) as a white solid. LC/MS (ESI) m/z: 742 (M+H)⁺.

### Step 7: (S)-N-((S)-1-(1H-pyrrolo[3,2-c]pyridin-2-yl)ethyl)-7-((9,9-difluoro-9H-fluorene-3-carbonyl)glycyl)-1,4-dioxa-7-azaspiro[4.4]nonane-8-carboxamide (Compound 23)

To a solution of (S)-7-((9,9-difluoro-9H-fluorene-3-carbonyl)glycyl)-N-((S)-1-(1-(phenylsulfonyl)-1H-pyrrolo[3,2-c]pyridin-2-yl)ethyl)-1,4-dioxa-7-azaspiro[4.4]nonane-8-carboxamide (10 mg, 0.014 mmol) in MeOH/H₂O (0.5 mL, v/v=1/1) was added LiOH·H₂O ((12 mg, 0.30 mmol) and the mixture was stirred at room temperature for 2 hours. The mixture was purified by prep-IIPLC to give Compound 23 (3 mg, yield 18.5%) as a white solid. ¹H NMR (400 MHz, CD₃OD): δ 8.50 (s, 1H), 7.91-7.84 (m, 2H), 7.69 (dd, J=7.8, 1.3 Hz, 1H), 7.65-7.53 (m, 4H), 7.44 (t, J=7.3 Hz, 1H), 7.23 (d, J=6.0 Hz, 1H), 6.58 (d, J=43.6 Hz, 1H), 5.35 (d, J=7.0 Hz, 1H), 4.65 (dd, J=9.1, 5.3 Hz, 1H), 4.27 (d, J=16.1 Hz, 1H), 4.08-4.01 (m, 5H), 3.97 (d, J=10.7 Hz, 1H), 3.85 (d, J=10.7 Hz, 1H), 2.51 (dd, J=13.2, 9.4 Hz, 1H), 2.28 (dd, J=13.2, 5.4 Hz, 1H), 2.03 (s, 1H), 1.70 (d, J=7.0 Hz, 3H). LC/MS (ESI) m/z: 602 (M+H)⁺. RT (Method A): 1.37 min.

### Scheme 8. Synthesis of (2S,4R)-N-((S)-1-(1H-pyrrolo[3,2-c]pyridin-2-yl)ethyl)-4-fluoro-4-(methoxymethyl)-1-((4-phenoxybenzoyl)glycyl)pyrrolidine-2-carboxamide (Compound 21)

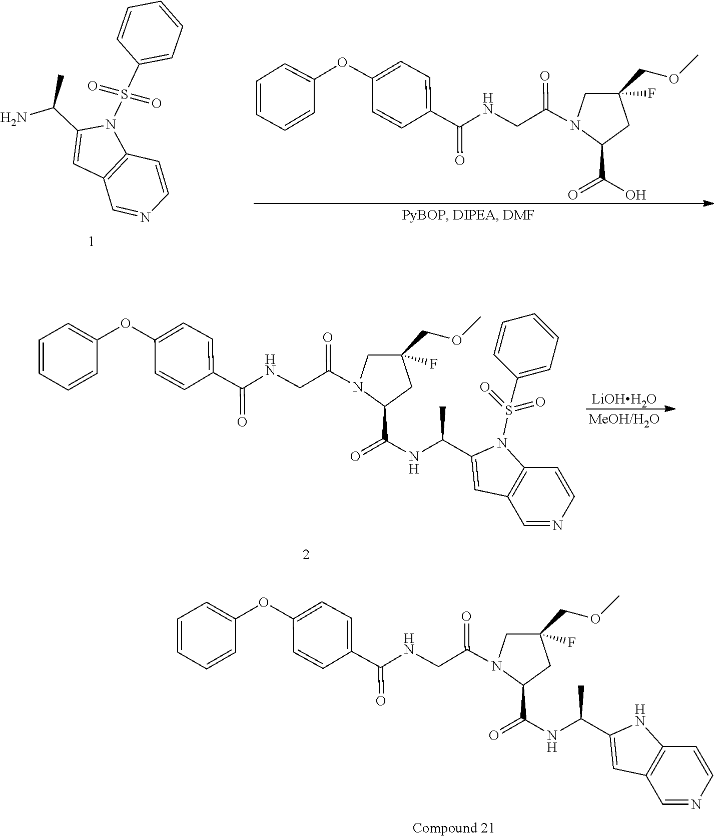 [SMILES: C[C@H](N)c1cc2cnccc2n1S(=O)(=O)c1ccccc1 ; COC[C@@]1(F)C[C@@H](C(=O)O)N(C(=O)CNC(=O)c2ccc(Oc3ccccc3)cc2)C1 ; COC[C@@]1(F)C[C@@H](C(=O)N[C@@H](C)c2cc3cnccc3n2S(=O)(=O)c2ccccc2)N(C(=O)CNC(=O)c2ccc(Oc3ccccc3)cc2)C1 ; COC[C@@]1(F)C[C@@H](C(=O)N[C@@H](C)c2cc3cnccc3[nH]2)N(C(=O)CNC(=O)c2ccc(Oc3ccccc3)cc2)C1]

### Step 1: (2S,4R)-4 fluoro-4-(methoxymethyl)-1-((4-phenoxybenzoyl)glycyl)-N-((S)-1-(1-(phenylsulfonyl)-1H-pyrrolo[3,2-c]pyridin-2-yl)ethyl)pyrrolidine-2-carboxamide

To a mixture of (R)-1-(1-(phenylsulfonyl)-1H-pyrrolo[3,2-c]pyridin-2-yl)ethan-1-amine (25 mg, 0.085 mmol) and (S)-1-(1-(phenylsulfonyl)-1H-pyrrolo[3,2-c]pyridin-2-yl)ethan-1-amine (36 mg, 0.085 mmol) in DMF (0.7 mL) was added DIPEA (44 mg, 0.34 mmol) and PyBOP (44 mg, 0.085 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified chromatography (silica gel, 0-10% MeOH in DCM) to give (2S,4R)-4-fluoro-4-(methoxymethyl)-1-((4-phenoxybenzoyl)glycyl)-N-((S)-1-(1-(phenylsulfonyl)-1H-pyrrolo[3,2-c]pyridin-2-yl) ethyl)pyrrolidine-2-carboxamide (10 mg, yield 16.5%) as a white solid. LC/MS (ESI) m/z: 714 (M+H)⁺.

### Step 2: (2S,4R)-N-((S)-1-(1H-pyrrolo[3,2-c]pyridin-2-yl)ethyl)-4-fluoro-4-(methoxymethyl)-1-((4-phenoxybenzoyl)glycyl)pyrrolidine-2-carboxamide (Compound 21)

To a solution of (2S,4R)-4-fluoro-4-(methoxymethyl)-1-((4-phenoxybenzoyl)glycyl)-N-((S)-1-(1-(phenylsulfonyl)-1H-pyrrolo[3,2-c]pyridin-2-yl)ethyl)pyrrolidine-2-carboxamide (10 mg, 0.014 mmol) in MeOH/H₂O (0.5 mL, v/v=1/1) was added LiOH·H₂O (12 mg, 0.30 mmol) and the mixture was stirred at room temperature for 2 hours. The mixture was purified by prep-HPLC to give Compound 21 (5.7 mg, yield 71.0%) as a white solid. ¹H NMR (400 MHz, CD₃OD): δ 8.66 (s, 1H), 8.05 (t, J=18.1 Hz, 1H), 7.77-7.73 (m, 2H), 7.44-7.40 (m, 2H), 7.31 (d, J=5.9 Hz, 1H), 7.21 (t, J=7.4 Hz, 1H), 7.07-7.04 (m, 2H), 6.93-6.90 (m, 2H), 6.57 (d, J=29.4 Hz, 1H), 5.32-5.26 (m, 1H), 4.63 (t, J=7.9 Hz, 1H), 4.18 (s, 2H), 4.08-3.93 (m, 2H), 3.71 (dd, J=17.8, 5.3 Hz, 2H), 3.43 (s, 3H), 2.64-2.54 (m, 1H), 2.21 (ddd, 1=20.2, 14.2, 7.0 Hz, 1H), 1.65 (d, J=7.0 Hz, 3H). LC/MS (EST) m/z: 574 (M+H)⁺. RT (Method A): 1.35 min.

The following compounds were prepared based on Scheme 8:

| Cmpd # | Reactant A | Reactant B | Characterization Data |
|---|---|---|---|
| 22 | 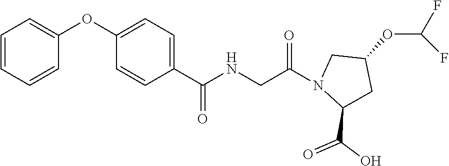 [SMILES: O=C(NCC(=O)N1C[C@H](OC(F)F)C[C@H]1C(=O)O)c1ccc(Oc2ccccc2)cc1] | 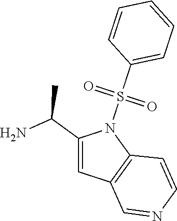 | ¹H NMR (400 MHz, CD₃OD): δ 8.71 (d, J = 33.2 Hz, 1H), 8.08 (dd, J = 32.8, 5.9 Hz, 1H), 7.88-7.74 (m, 2H), 7.44-7.40 (m, 2H), 7.32 (d, J = 5.9 Hz, 1H), 7.21 (t, J = 7.4 Hz, 1H), 7.05 (dd, J = 8.6, 1.0 Hz, 2H), 6.94-6.89 (m, 2H), 6.71-6.34 (m, 2H), 5.29 (q, J = 6.9 Hz, 1H), 5.02 (s, 1H), 4.59 (s, 1H), 4.21 (dd, J = 16.4, 7.8 Hz, 2H), 4.02 (dt, J = 9.2, 4.6 Hz, 1H), 3.92 (t, J = 14.6 Hz, 1H), 2.56-2.47 (m, 1H), 2.32-2.23 (m, 1H), 1.66 (dd, J = 16.3, 7.0 Hz, 3H). LC/MS (ESI) m/z: 578 (M + H)⁺. RT (Method A): 1.44 min. |
| 38 | 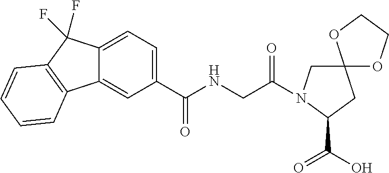 [SMILES: O=C(NCC(=O)N1CC2(C[C@H]1C(=O)O)OCCO2)c1ccc2c(c1)-c1ccccc1C2(F)F] | 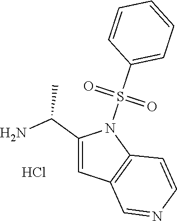 | ¹H NMR (400 MHz, CD₃OD): δ 8.90 (d, J = 34.1 Hz, 1H), 8.42 (s, 1H), 8.18 (s, 1H), 7.97 (d, J = 6.5 Hz, 1H), 7.90 (d, J = 7.9 Hz, 1H), 7.75-7.69 (m, 2H), 7.66 (d, J = 7.3 Hz, 1H), 7.58-7.50 (m, 2H), 7.45 (t, J = 7.5 Hz, 1H), 6.90 (d, J = 23.9 Hz, 1H), 5.33 (q, J = 6.7 Hz, 1H), 4.57 (t, J = 8.0 Hz, 1H), 4.25 (dd, J = 41.0, 16.8 Hz, 2H), 4.03 (dd, J = 9.9, 5.2 Hz, 4H), 3.82 (t, J = 7.0 Hz, 2H), 2.44 (dd, J = 13.0, 8.3 Hz, 1H), 2.27 (dd, J = 13.0, 7.8 Hz, 1H), 1.65 (t, J = 15.1 Hz, 3H). LC/MS (ESI) m/z: 602 (M + H)⁺. RT (Method A): 1.35 min. |
| 47^{a,d} | 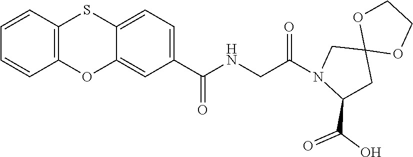 [SMILES: O=C(NCC(=O)N1CC2(C[C@H]1C(=O)O)OCCO2)c1ccc2c(c1)Oc1ccccc1S2] | 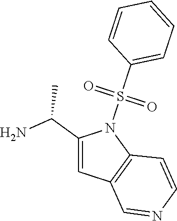 [SMILES: C[C@@H](N)c1cc2cnccc2n1S(=O)(=O)c1ccccc1] | ¹H NMR (400 MHz, CD₃OD): δ 8.66 (s, 1H), 7.93 (d, J = 5.9 Hz, 1H), 7.60- 7.47 (m, 2H), 7.26-7.13 (m, 4H), 7.08 (t, J = 7.4 Hz, 1H), 7.03 (d, J = 8.1 Hz, 1H), 6.55 (s, 1H), 5.31 (dd, J = 13.4, 6.8 Hz, 1H), 4.54 (t, J = 8.0 Hz, 1H), 4.19 (dd, J = 58.7, 16.8 Hz, 2H), 4.02 (d, J = 6.8 Hz, 4H), 3.79 (s, 2H), 2.40 (dd, J = 13.0, 8.4 Hz, 1H), 2.27 (dd, J = 13.2, 7.7 Hz, 1H), 1.59 (d, J = 6.9 Hz, 3H). LC/MS (ESI) m/z: 600 (M + H)⁺. RT (Method A): 1.23 min. |
| 56 | 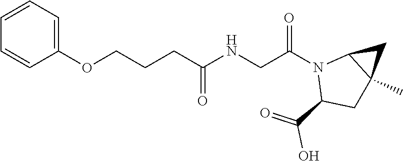 | 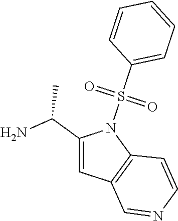 | ¹H NMR (400 MHz, CD₃OD): δ 8.87 (s, 1H), 8.16 (s, 1H), 7.66 (s, 1H), 7.19 (d, J = 8.3 Hz, 2H), 6.88-6.85 (m, 3H), 6.77 (s, 1H), 5.28-5.23 (m, 1H), 4.79 (d, J = 3.8 Hz, 1H), 4.32 (d, J = 16.8 Hz, 1H), 4.10 (d, J = 16.7 Hz, 1H), 4.00 (t, J = 6.3 Hz, 2H), 3.33 (s, 1H), 2.51 (t, J = 7.5 Hz, 2H), 2.40 (d, J = 11.5 Hz, 1H), 2.15 (dd, J = 13.4, 3.9 Hz, 1H), 2.09 (d, J = 6.5 Hz, 2H), 1.60 (d, J = 7.0 Hz, 3H), 1.30 (s, 4H), 0.82 (t, J = 5.4 Hz, 1H). LC/MS (ESI) m/z: 504 (M + H)⁺. RT (Method A): 1.14 min. |
| 57 | 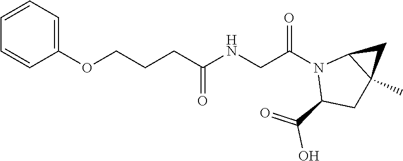 | 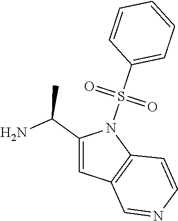 | ¹H NMR (400 MHz, CD₃OD): δ 8.91 (d, J = 12.6 Hz, 1H), 8.42 (s, 1H), 8.21 (d, J = 6.5 Hz, 1H), 7.73 (d, J = 6.4 Hz, 1H), 7.22 (t, J = 7.9 Hz, 2H), 6.91- 6.83 (m, 3H), 6.79 (s, 1H), 5.25 (q, J = 6.8 Hz, 1H), 4.84 (s, 1H), 4.15 (d, J = 2.3 Hz, 2H), 3.93 (t, J = 6.3 Hz, 2H), 3.36 (dd, J = 5.9, 2.2 Hz, 1H), 2.42 (dd, J = 12.6, 7.7 Hz, 3H), 2.22 (dd, J = 13.4, 3.3 Hz, 1H), 2.02-1.94 (m, 2H), 1.64 (d, J = 7.0 Hz, 3H), 1.30 (s, 3H), 1.15-1.11 (m, 1H), 0.84 (t, J = 5.9 Hz, 1H). LC/MS (ESI) m/z: 504 (M + H)⁺. RT (Method A): 1.16 min. |
| 71^{a,d} | 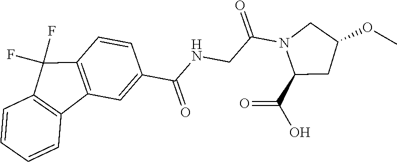 | 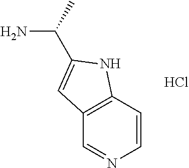 | ¹H NMR (400 MHz, CD₃OD): δ 8.72 (s, 1H), 8.51 (s, 1H), 8.23 (s, 1H), 7.94 (d, J = 7.8 Hz, 1H), 7.82 (d, J = 6.0 Hz, 1H), 7.77-7.71 (m, 2H), 7.67 (d, J = 7.5 Hz, 1H), 7.56 (t, J = 7.5 Hz, 1H), 7.45 (t, J = 7.5 Hz, 1H), 7.23 (d, J = 6.0 Hz, 1H), 6.66 (s, 1H), 5.32 (q, J = 7.0 Hz, 1H), 4.50 (t, J = 8.3 Hz, 1H), 4.41 (d, J = 16.8 Hz, 1H), 4.22 (d, J = 16.5 Hz, 2H), 3.84 (dt, J = 11.4, 7.4 Hz, 2H), 3.40 (s, 3H), 2.44 (dd, J = 13.6, 7.3 Hz, 1H), 2.17-2.06 (m, 1H), 1.61 (d, J = 6.9 Hz, 3H). LC/MS (ESI) m/z: 574 (M + H)⁺. RT (Method A): 1.25 min. |
| 72^{a} | 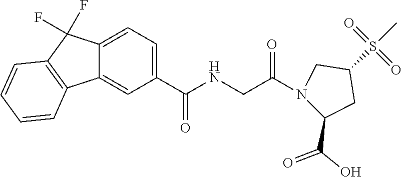 | 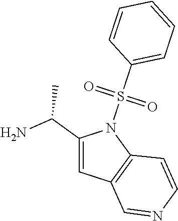 | ¹H NMR (400 MHz, CD₃OD): δ 8.84 (s, 1H), 8.18 (s, 1H), 7.98 (s, 1H), 7.90 (d, J = 7.4 Hz, 1H), 7.72 (d, J = 7.4 Hz, 2H), 7.65 (s, 1H), 7.56 (s, 1H), 7.52 (d, J = 5.3 Hz, 1H), 7.45 (s, 1H), 6.83 (s, 1H), 5.32 (d, J = 5.3 Hz, 1H), 4.73 (s, 1H), 4.35 (s, 1H), 4.22 (d, J = 6.0 Hz, 3H), 3.11 (s, 3H), 2.81 (s, 1H), 2.50 (s, 1H), 1.63 (d, J = 6.2 Hz, 2H). LC/MS (ESI) m/z: 622 (M + H)⁻. RT (Method A): 1.22 min. |
| 73^{a,d} | 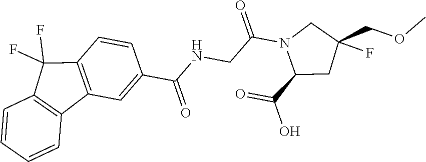 | 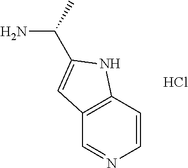 | ¹H NMR (400 MHz, CD₃OD): δ 8.85 (s, 1H), 8.22 (s, 1H), 7.91 (dd, J = 17.6, 7.1 Hz, 2H), 7.74 (dd, J = 14.2, 7.5 Hz, 2H), 7.67 (d, J = 7.3 Hz, 1H), 7.57 (t, J = 7.5 Hz, 1H), 7.45 (dd, J = 11.2, 7.2 Hz, 2H), 6.85 (s, 1H), 5.37-5.30 (m, 1H), 4.67-4.61 (m, 1H), 4.40 (d, J = 16.6 Hz, 1H), 4.21 (d, J = 16.9 Hz, 1H), 4.12-4.03 (m, 1H), 4.03-3.89 (m, 1H), 3.74 (dd, J = 17.5, 5.2 Hz, 2H), 3.45 (s, 3H), 2.55 (td, J = 15.1, 7.2 Hz, 1H), 2.36-2.19 (m, 1H), 1.64 (d, J = 7.0 Hz, 3H). LC/MS (ESI) m/z: 606 (M + H)⁺. RT (Method A): 1.46 min. |
| 74^{a,d} | 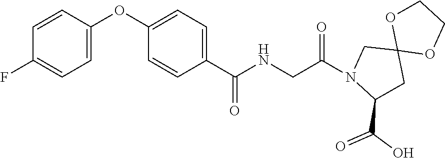 [SMILES: O=C(NCC(=O)N1CC2(C[C@H]1C(=O)O)OCCO2)c1ccc(Oc2ccc(F)cc2)cc1] | 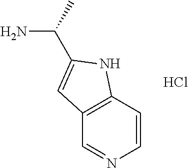 | ¹H NMR (400 MHz, CD₃OD): δ 8.70 (d, J = 10.1 Hz, 1H), 7.94 (d, J = 5.9 Hz, 1H), 7.87 (t, J = 11.2 Hz, 2H), 7.17 (t, J = 7.5 Hz, 3H), 7.11 (dd, J = 8.4, 4.8 Hz, 2H), 7.00 (d, J = 8.5 Hz, 2H), 6.58 (s, 1H), 5.31 (q, J = 7.1 Hz, 1H), 4.54 (t, J = 8.0 Hz, 1H), 4.25 (d, J = 16.6 Hz, 1H), 4.13 (d, J = 16.5 Hz, 1H), 4.01 (dd, J = 15.5, 8.9 Hz, 4H), 3.84-3.72 (m, 2H), 2.40 (dd, J = 12.8, 8.3 Hz, 1H), 2.26 (dd, J = 13.1, 7.6 Hz, 1H), 1.59 (d, J = 7.0 Hz, 3H). LC/MS (ESI) m/z: 588 (M + H)⁺. RT (Method A): 1.24 min. |
| 75^{a} | 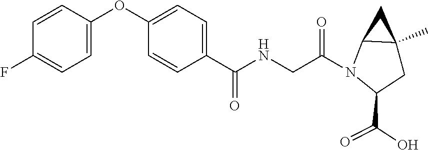 | 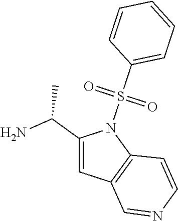 | ¹H NMR (400 MHz, CD₃OD): δ 8.74 (s, 1H), 7.92 (d, J = 8.2 Hz, 3H), 7.24- 7.09 (m, 5H), 7.03 (d, J = 8.3 Hz, 2H), 6.64 (s, 1H), 5.34-5.30 (m, 1H), 4.65 (s, 1H), 4.36 (d, J = 16.8 Hz, 1H), 4.15 (d, J = 16.8 Hz, 2H), 2.65-2.54 (m, 1H), 2.34-2.24 (m, 1H), 1.62 (d, J = 6.9 Hz, 3H), 1.30-1.29 (m, 4H), 0.90 (t, 1H). LC/MS (ESI) m/z: 556 (M + H)⁺. RT (Method A): 1.39 min. |
| 79^{a,d} | 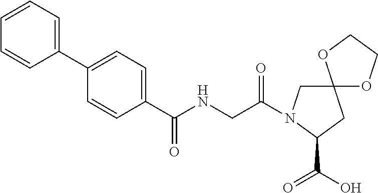 [SMILES: O=C(NCC(=O)N1CC2(C[C@H]1C(=O)O)OCCO2)c1ccc(-c2ccccc2)cc1] | 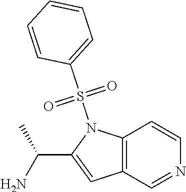 | ¹H NMR (400 MHz, CD₃OD): δ 8.65 (s, 1H), 7.98 (d, J = 7.9 Hz, 2H), 7.87 (s, 1H), 7.75 (d, J = 7.4 Hz, 2H), 7.69 (d, J = 7.6 Hz, 2H), 7.47 (d, J = 7.3 Hz, 2H), 7.40 (s, 1H), 7.12 (s, 1H), 6.54 (s, 1H), 5.32 (d, J = 7.3 Hz, 1H), 4.55 (t, J = 8.1 Hz, 1H), 4.30 (d, J = 16.7 Hz, 1H), 4.16 (d, J = 17.7 Hz, 1H), 4.02 (d, J = 7.2 Hz, 4H), 3.82 (d, J = 11.3 Hz, 2H), 2.44-2.37 (m, 1H), 2.28 (dd, J = 11.1, 8.2 Hz, 1H), 1.60 (d, J = 7.0 Hz, 3H). LC/MS (ESI) m/z: 554 (M + H)⁺. RT (Method A): 1.15 min. |
| 80^{a,d} | 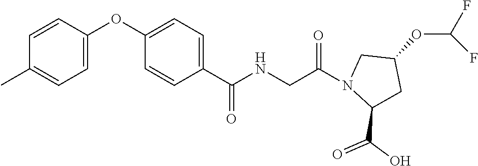 | 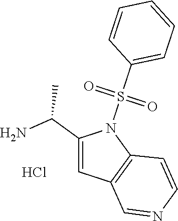 | ¹H NMR (400 MHz, CD₃OD): δ 8.94 (s, 1H), 8.33 (s, 1H), 8.05 (d, J = 6.1 Hz, 1H), 7.89 (d, J = 7.9 Hz, 2H), 7.55 (d, J = 5.9 Hz, 1H), 7.24 (d, J = 7.2 Hz, 2H), 7.02-6.97 (m, 4H), 6.90 (s, 1H), 6.53 (t, J = 74.2 Hz, 1H), 5.33 (qd, J = 6.3, 1.2 Hz, 1H), 5.09-5.05 (m, 1H), 4.59 (t, J = 7.7 Hz, 1H), 4.34-4.29 (m, 1H), 4.21-4.16 (m, 1H), 3.98-3.90 (m, 2H), 2.52-2.46 (m, 1H), 2.36 (s, 3H), 2.31-2.25 (m, 1H), 1.64 (d, J = 6.7 Hz, 3H). LC/MS (ESI) m/z: 592 (M + H)⁺. RT (Method A): 1.46 min. |
| 81^{a,d} | 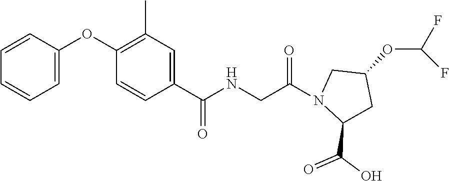 | 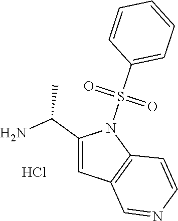 | ¹H NMR (400 MHz, CD₃OD): δ 8.89 (s, 1H), 8.42 (s, 1H), 8.01 (d, J = 5.6 Hz, 1H), 7.86 (s, 1H), 7.72 (d, J = 7.9 Hz, 1H), 7.50-7.44 (m, 1H), 7.41-7.36 (m, 2H), 7.16 (ddd, J = 7.5, 6.6, 1.2 Hz, 1H), 6.99 (d, J = 7.9 Hz, 2H), 6.88-6,85 (m, 1H), 6.84-6.81 (m, 1H), 6.44 (d, J = 73.2 Hz, 1H), 5.35-5.30 (m, 1H), 5.07 (pd, J = 3.8, 0.8 Hz, 1H), 4.61-4.57 (m, 1H), 4.35-4.30 (m, 1H), 4.22- 4.16 (m, 1H), 3.95 (ddd, J = 14.7, 1.6, 0.8 Hz, 2H), 2.52-2.46 (m, 1H), 2.33 (s, 3H), 2.27 (ddd, J = 7.6, 4.3, 1.8 Hz, 1H), 1.64 (d, J = 6.1 Hz, 3H). LC/MS (ESI) m/z: 592 (M + H)⁺. RT (Method A): 1.44 min. |
| 82^{a,d} | 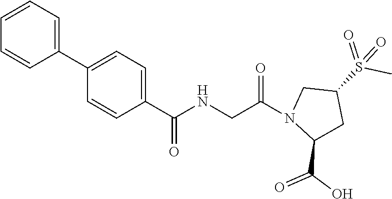 | 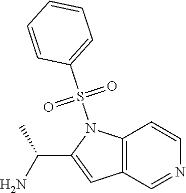 | ¹H NMR (400 MHz, CD₃OD): δ 8.65 (s, 1H), 7.99 (d, J = 7.8 Hz, 2H), 7.89 (d, J = 5.2 Hz, 1H), 7.76 (d, J = 7.2 Hz, 2H), 7.70 (d, J = 7.3 Hz, 2H), 7.48 (d, J = 7.1 Hz, 2H), 7.40 (s, 1H), 7.13 (d, J = 5.4 Hz, 1H), 6.53 (s, 1H), 5.29 (d, J = 6.4 Hz, 1H), 4.70 (s, 1H), 4.35 (t, J = 13.2 Hz, 2H), 4.17 (d, J = 24.3 Hz, 4H), 3.09 (s, 3H), 2.77 (dd, J = 14.2, 7.5 Hz, 1H), 2.50-2.43 (m, 1H), 1.60 (d, J = 6.3 Hz, 3H). LC/MS (ESI) m/z: 574 (M + H)⁺. RT (Method A): 1.07 min. |
| 83^{a,d} | 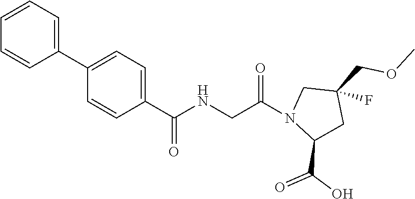 | 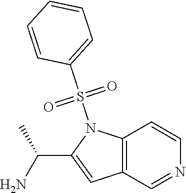 | ¹H NMR (400 MHz, CD₃OD): δ 8.64 (s, 1H), 8.01 (d, J = 7.6 Hz, 2H), 7.79 (dd, J = 13.4, 6.7 Hz, 3H), 7.71 (d, J = 7.3 Hz, 2H), 7.49 (t, J = 7.4 Hz, 2H), 7.41 (d, J = 7.3 Hz, 1H), 7.03 (d, J = 5.2 Hz, 1H), 6.52 (s, 1H), 5.31 (d, J = 6.8 Hz, 1H), 4.62 (t, J = 8.2 Hz, 1H), 4.40 (d, J = 16.8 Hz, 1H), 4.17 (d, J = 17.0 Hz, 1H), 4.09-3.88 (m, 2H), 3.78-3.69 (m, 2H), 3.45 (s, 3H), 2.60-2.47 (m, 1H), 2.34-2.15 (m, 1H), 1.61 (d, J = 6.5 Hz, 3H). LC/MS (ESI) m/z: 558 (M + H)⁺. RT (Method A): 1.21 min. |
| 84^{a,d} | 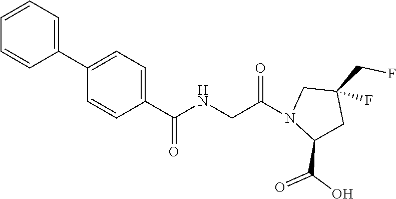 [SMILES: O=C(NCC(=O)N1C[C@@](F)(CF)C[C@H]1C(=O)O)c1ccc(-c2ccccc2)cc1] | 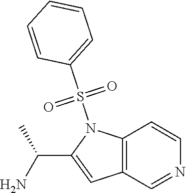 | ¹H NMR (400 MHz, CD₃OD): δ 8.65 (s, 1H), 8.01 (d, J = 8.3 Hz, 2H), 7.85- 7.76 (m, 3H), 7.71 (d, J = 7.2 Hz, 2H), 7.48 (d, J = 7.8 Hz, 2H), 7.41 (d, J = 7.6 Hz, 1H), 7.05 (s, 1H), 6.55 (s, 1H), 5.31 (d, J = 7.3 Hz, 1H), 4.78 (s, 1H), 4.67 (d, J = 9.2 Hz, 2H), 4.41 (d, J = 17.6 Hz, 1H), 4.18 (d, J = 15.1 Hz, 2H), 4.00 (dd, J = 32.8, 11.5 Hz, 1H), 2.59 (ddd, J = 15.4, 12.2, 7.8 Hz, 1H), 2.34- 2.20 (m, 1H), 1.61 (d, J = 6.0 Hz, 3H). LC/MS (ESI) m/z: 546 (M + H)⁺. RT (Method A): 1.23 min. |
| 86^{a,d} | 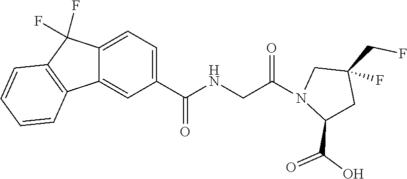 [SMILES: O=C(NCC(=O)N1C[C@@](F)(CF)C[C@H]1C(=O)O)c1ccc2c(c1)-c1ccccc1C2(F)F] | 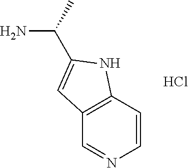 | ¹H NMR (400 MHz, CD₃OD): δ 8.83 (s, 1H), 8.21 (s, 1H), 7.91 (dd, J = 13.8, 6.9 Hz, 2H), 7.74 (dd, J = 13.1, 7.6 Hz, 2H), 7.66 (s, 1H), 7.55 (d, J = 7.6 Hz, 1H), 7.47-7.41 (m, 2H), 6.83 (s, 1H), 5.34 (q, J = 6.9 Hz, 1H), 4.79 (s, 2H), 4.71 (d, J = 7.4 Hz, 1H), 4.41 (d, J = 16.8 Hz, 1H), 4.22 (d, J = 16.8 Hz, 1H), 4.18-4.10 (m, 1H), 4.02 (dd, J = 32.7, 12.1 Hz, 1H), 2.60 (dd, J = 16.3, 7.4 Hz, 1H), 2.37-2.21 (m, 1H), 1.63 (d, J = 6.9 Hz, 3H). LC/MS (ESI) m/z: 594 (M + H)⁺. RT (Method A): 1.35 min. |
| 87^{a,da} | 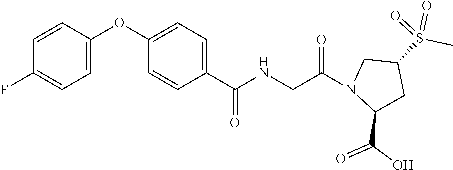 | 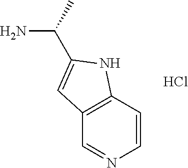 | ¹H NMR (400 MHz, CD₃OD): δ 8.67 (d, J = 12.4 Hz, 1H), 8.06-7.85 (m, 3H), 7.20-7.08 (m, 5H), 7.01 (d, J = 8.6 Hz, 2H), 6.56 (d, J = 29.6 Hz, 1H), 5.34-5.22 (m, 1H), 4.83-4.76 (m, 1H), 4.72-4.65 (m, 1H), 4.29 (q, J = 17.2 Hz, 2H), 4.18 (s, 2H), 3.05 (d, J = 22.0 Hz, 3H), 2.76 (s, 1H), 2.56-2.37 (m, 1H), 1.60 (d, J = 6.9 Hz, 3H). LC/MS (ESI) m/z: 608 (M + H)⁺. RT (Method A): 1.15 min. |
| 88^{a,d} | 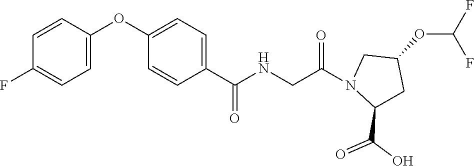 [SMILES: O=C(NCC(=O)N1C[C@H](OC(F)F)C[C@H]1C(=O)O)c1ccc(Oc2ccc(F)cc2)cc1] | 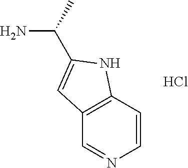 | ¹H NMR (400 MHz, CD₃OD): δ 8.69 (d, J = 9.0 Hz, 1H), 7.94-7.86 (m, 3H), 7.17 (t, J = 8.6 Hz, 2H), 7.12 (d, J = 4.9 Hz, 2H), 7.02 (d, J = 8.6 Hz, 2H), 6.71-6.34 (m, 2H), 5.30 (q, J = 6.6 Hz, 1H), 5.05 (s, 1H), 4.57 (t, J = 8.0 Hz, 1H), 4.34 (d, J = 16.8 Hz, 1H), 4.15 (d, J = 16.8 Hz, 1H), 3.97 (dd, J = 11.6, 4.1 Hz, 1H), 3.89 (d, J = 11.5 Hz, 1H), 2.51-2.43 (m, 1H), 2.26 (ddd, J = 13.3, 8.5, 4.8 Hz, 1H), 1.60 (d, J = 7.0 Hz, 3H). LC/MS (ESI) m/z: 596 (M + H)⁺. RT (Method A): 1.33 min. |
| 92^{a} | 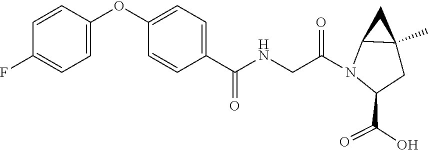 | 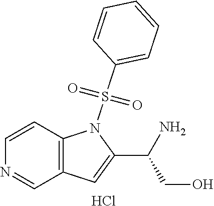 [SMILES: N[C@H](CO)c1cc2cnccc2n1S(=O)(=O)c1ccccc1 ; Cl] | ¹H NMR (400 MHz, CD₃OD): δ 8.66 (s, 1H), 7.94 (s, 1H), 7.89 (d, J = 7.8 Hz, 2H), 7.19-7.05 (m, 5H), 7.00 (d, J = 7.8 Hz, 2H), 6.56 (s, 1H), 5.27-5.15 (m, 1H), 4.93-4.89 (m, 1H), 4.52-4.44 (m, 1H), 4.27 (d, J = 16.0 Hz, 1H), 4.03- 3.95 (m, 1H), 3.91-3.83 (m, 1H), 3.42-3.36 (m, 1H), 2.49-2.38 (m, 1H), 2.29-2.19 (m, 1H), 1.32-1.28 (m, 4H), 0.85-0.78 (m, 1H). LC/MS (ESI) m/z: 572 (M + H)⁺. RT (Method A): 1.29 min. |
| 93^{a} | 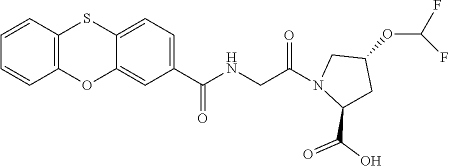 [SMILES: O=C(NCC(=O)N1C[C@H](OC(F)F)C[C@H]1C(=O)O)c1ccc2c(c1)Oc1ccccc1S2] | 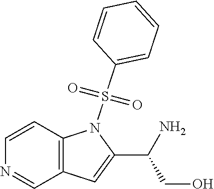 [SMILES: N[C@H](CO)c1cc2cnccc2n1S(=O)(=O)c1ccccc1] | ¹H NMR (400 MHz, CD₃OD): δ 8.66 (d, J = 14.6 Hz, 1H), 7.97 (dd, J = 60.6, 5.7 Hz, 1H), 7.58 (dd, J = 11.0, 4.8 Hz, 2H), 7.26 (d, J = 8.0 Hz, 1H), 7.21- 7.15 (m, 2H), 7.10 (dd, J = 15.8, 6.6 Hz, 2H), 7.03 (d, J = 8.0 Hz, 1H), 6.54 (dd, J = 81.5, 67.6 Hz, 2H), 5.32-5.26 (m, 1H), 5.06 (s, 1H), 4.65 (t, J = 8.0 Hz, 1H), 4.34 (d, J = 16.8 Hz, 1H), 4.14 (d, J = 16.8 Hz, 1H), 4.05-3.95 (m, 2H), 3.88 (dd, J = 11.3, 6.8 Hz, 2H), 2.55-2.45 (m, 1H), 2.32 (ddd, J = 13.6, 8.5, 4.8 Hz, 1H). LC/MS (ESI) m/z: 624 (M + H)⁺. RT (Method A): 1.35 min. |
| 94^{a} | 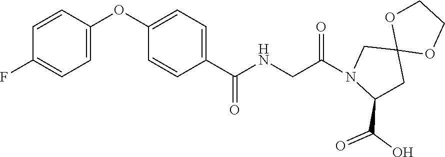 [SMILES: O=C(NCC(=O)N1CC2(C[C@H]1C(=O)O)OCCO2)c1ccc(Oc2ccc(F)cc2)cc1] | 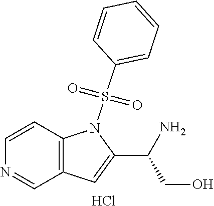 [SMILES: N[C@H](CO)c1cc2cnccc2n1S(=O)(=O)c1ccccc1 ; Cl] | ¹H NMR (400 MHz, CD₃OD): δ 8.68 (d, J = 10.5 Hz, 1H), 7.95 (d, J = 5.8 Hz, 1H), 7.88 (t, J = 7.2 Hz, 2H), 7.17 (ddt, J = 9.0, 5.8, 2.8 Hz, 3H), 7.13-7.09 (m, 2H), 7.01 (d, J = 8.7 Hz, 2H), 6.63 (d, J = 22.0 Hz, 1H), 5.31 (dt, J = 24.8, 5.6 Hz, 1H), 4.62 (t, J = 7.9 Hz, 1H), 4.25 (d, J = 16.7 Hz, 1H), 4.14 (d, J = 16.7 Hz, 1H), 4.05-3.94 (m, 6H), 3.88 (dd, J = 11.4, 6.6 Hz, 1H), 3.80 (s, 1H), 2.43 (dd, J = 13.1, 8.4 Hz, 1H), 2.29 (dd, J = 13.1, 7.4 Hz, 1H). LC/MS (ESI) m/z: 604 (M + H)⁺. RT (Method A): 1.16 min. |
| 96^{a} | 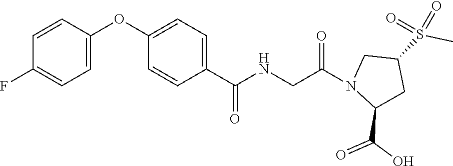 | 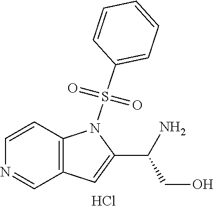 [SMILES: N[C@H](CO)c1cc2cnccc2n1S(=O)(=O)c1ccccc1 ; Cl] | ¹H NMR (400 MHz, CD₃OD): δ 8.68 (d, J = 11.2 Hz, 1H), 7.95 (d, J = 5.8 Hz, 1H), 7.88 (t, J = 9.7 Hz, 2H), 7.20 (d, J = 4.8 Hz, 1H), 7.16 (d, J = 8.2 Hz, 2H), 7.13-7.09 (m, 2H), 7.01 (d, J = 8.9 Hz, 2H), 6.61 (d, J = 22.0 Hz, 1H), 5.30-5.23 (m, 1H), 4.79-4.76 (m, 2H), 4.29 (d, J = 9.0 Hz, 2H), 4.18 (s, 2H), 4.03-3.99 (m, 1H), 3.88 (dd, J = 11.3, 6.9 Hz, 1H), 3.08 (s, 3H), 2.83-2.70 (m, 1H), 2.59-2.45 (m, 1H). LC/MS (ESI) m/z: 624 (M + H)⁺. RT (Method A): 1.10 min. |
| 97^{a} | 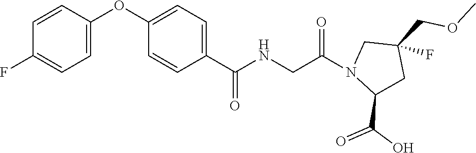 | 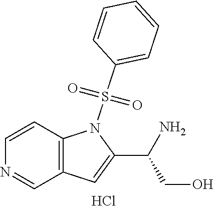 [SMILES: N[C@H](CO)c1cc2cnccc2n1S(=O)(=O)c1ccccc1 ; Cl] | ¹H NMR (400 MHz, CD₃OD): δ 8.66 (d, J = 0.9 Hz, 1H), 7.93-7.88 (m, 3H), 7.14 (ddt, J = 6.8, 4.6, 2.4 Hz, 4H), 7.09 (d, J = 6.1 Hz, 1H), 7.03 (d, J = 8.9 Hz, 2H), 6.57 (s, 1H), 5.30-5.26 (m, 1H), 4.72-4.67 (m, 1H), 4.35 (d, J = 16.7 Hz, 1H), 4.12 (d, J = 16.8 Hz, 1H), 4.05-3.99 (m, 2H), 3.88 (dd, J = 5.6, 4.0 Hz, 1H), 3.74 (d, J = 3.7 Hz, 1H), 3.70 (d, J = 6.6 Hz, 1H), 3.48 (dt, J = 3.3, 1.7 Hz, 1H), 3.44 (s, 3H), 3.13 (dt, J = 3.2, 1.6 Hz, 1H). LC/MS (ESI) m/z: 608 (M + H)⁺. RT (Method A): 1.20 min. |
| 98^{a} | 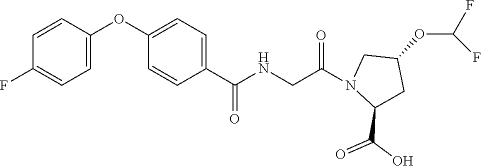 [SMILES: O=C(NCC(=O)N1C[C@H](OC(F)F)C[C@H]1C(=O)O)c1ccc(Oc2ccc(F)cc2)cc1] | 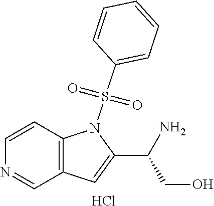 [SMILES: N[C@H](CO)c1cc2cnccc2n1S(=O)(=O)c1ccccc1 ; Cl] | ¹H NMR (400 MHz, CD₃OD): δ 8.66 (s, 1H), 7.93-7.83 (m, 3H), 7.20-7.09 (m, 5H), 7.04-6.97 (m, 2H), 6.72-6.34 (m, 2H), 5.27 (t, J = 5.9 Hz, 1H), 5.06 (s, 1H), 4.65 (t, J = 8.0 Hz, 1H), 4.33 (d, J = 16.8 Hz, 1H), 4.15 (d, J = 16.8 Hz, 1H), 4.00 (ddd, J = 18.9, 11.4, 4.7 Hz, 2H), 3.88 (dd, J = 11.3, 6.8 Hz, 2H), 2.54-2.45 (m, 1H), 2.32 (ddd, J = 13.5, 8.4, 4.9 Hz, 1H). LC/MS (ESI) m/z: 612 (M + H)⁺. RT (Method A): 1.10 min. |
| 99^{a} | 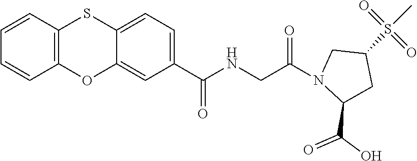 | 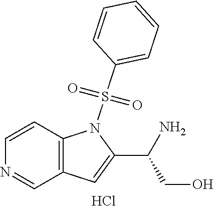 [SMILES: N[C@H](CO)c1cc2cnccc2n1S(=O)(=O)c1ccccc1 ; Cl] | ¹H NMR (400 MHz, CD₃OD): δ 8.66 (d, J = 0.8 Hz, 1H), 7.95 (d, J = 5.8 Hz, 1H), 7.58 (dd, J = 7.7, 1.5 Hz, 1H), 7.54 (d, J = 1.8 Hz, 1H), 7.26 (s, 1H), 7.22 (s, 1H), 7.20 (s, 1H), 7.15 (s, 1H), 7.08 (dd, J = 11.2, 3.8 Hz, 1H), 7.04 (s, 1H), 6.58 (s, 1H), 5.27 (t, J = 5.7 Hz, 1H), 4.83-4.66 (m, 2H), 4.34 (s, 1H), 4.27 (s, 1H), 4.23-4.14 (m, 3H), 4.01 (dd, J = 11.3, 5.0 Hz, 1H), 3.88 (dd, J = 11.3, 6.8 Hz, 1H), 3.18-2.95 (m, 3H), 2.87-2.68 (m, 1H), 2.59-2.41 (m, 1H). LC/MS (ESI) m/z: 636 (M + H)⁺. RT (Method A): 1.16 min. |
| 102^{b} | 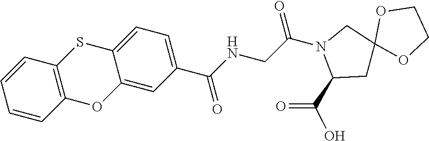 [SMILES: O=C(NCC(=O)N1CC2(C[C@H]1C(=O)O)OCCO2)c1ccc2c(c1)Oc1ccccc1S2] | 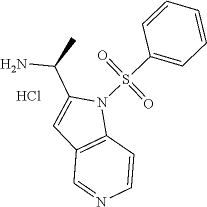 [SMILES: C[C@H](N)c1cc2cnccc2n1S(=O)(=O)c1ccccc1 ; Cl] | ¹H NMR (400 MHz, CD₃OD): δ 8.63 (s, 1H), 8.00 (d, J = 6.5 Hz, 1H), 7.46 (d, J = 6.5 Hz, 1H), 7.33 (dd, J = 8.1, 1.8 Hz, 1H), 7.24-7.21 (m, 1H), 7.17- 7.14 (m, 2H), 7.12-7.09 (m, 1H), 7.04-7.00 (m, 2H), 6.70 (s, 1H), 5.41- 5.35 (m, 1H), 4.66 (dd, J = 9.3, 4.8 Hz, 1H), 4.25 (d, J = 15.9 Hz, 1H), 4.06- 4.02 (m, 4H), 4.01-3.99 (m, 1H), 3.91 (d, J = 15.8 Hz, 1H), 3.84 (d, J = 10.6 Hz, 1H), 2.52 (dd, J = 13.2, 9.4 Hz, 1H), 2.29 (dd, J = 13.3, 4.8 Hz, 1H), 1.72 (d, J = 7.0 Hz, 3H). LC/MS (ESI) m/z: 600 (M + H)⁺. RT (Method A): 1.35 min. |
| 104^{a} | 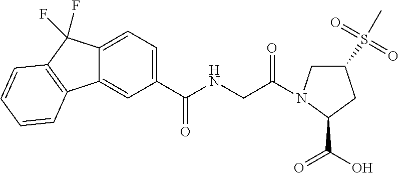 | 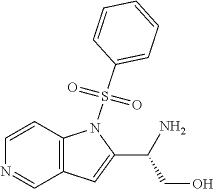 [SMILES: N[C@H](CO)c1cc2cnccc2n1S(=O)(=O)c1ccccc1] | ¹H NMR (400 MHz, CD₃OD): δ 8.65 (d, J = 0.9 Hz, 1H), 8.22 (s, 1H), 7.92 (dd, J = 7.8, 1.4 Hz, 1H), 7.88 (d, J = 5.8 Hz, 1H), 7.78-7.70 (m, 3H), 7.66 (d, J = 7.1 Hz, 2H), 7.56 (t, J = 7.1 Hz, 1H), 7.44 (t, J = 7.5 Hz, 1H), 7.18 (d, J = 5.8 Hz, 1H), 6.58 (s, 1H), 5.28 (s, 1H), 4.85 (s, 61H), 4.35 (d, J = 13.1 Hz, 2H), 4.21 (s, 4H), 4.02 (dd, J = 11.3, 5.0 Hz, 2H), 3.88 (dd, J = 11.3, 6.8 Hz, 1H), 3.09 (q, J = 13.9 Hz, 4H), 2.79 (m, 1H), 2.55 (d, J = 5.3 Hz, 1H). LC/MS (ESI) m/z: 638 (M + H)⁺. RT (Method A): 1.20 min. |
| 115^{c,d} | 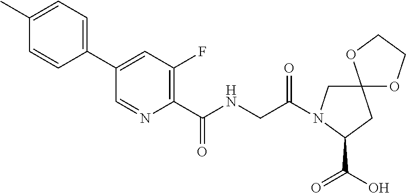 | 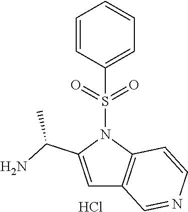 | ¹H NMR (400 MHz, DMSO-d6): δ 11.01 (s, 1H), 8.82 (s, 1H), 8.79 (s, 1H), 8.74 (t, J = 5.5 Hz, 1H), 8.63 (s, 1H), 8.50 (d, J = 8.5 Hz, 1H), 8.24 (s, 1H), 8.14 (t, J = 11.2 Hz, 1H), 7.89 (d, J = 5.6 Hz, 1H), 7.73 (t, J = 8.6 Hz, 1H), 7.53 (d, J = 6.5 Hz, 1H), 7.30 (t, J = 7.3 Hz, 1H), 7.09 (d, J = 5.6 Hz, 1H), 6.38 (s, 1H), 5.11 (h, J = 7.6 Hz, 1H), 4.63-4.17 (m, 2H), 4.04 (dd, J = 17.1, 5.5 Hz, 1H), 3.75 (d, J = 11.0 Hz, 1H), 3.59 (d, J = 10.7 Hz, 1H), 2.43 (s, 3H), 2.25 (dd, J = 13.1, 8.3 Hz, 1H), 2.07 (dd, J = 12.9, 8.0 Hz, 1H), 1.41 (d, J = 6.9 Hz, 3H). LC/MS (ESI) m/z: 587(M + H)⁺. RT (Method A): 1.41 min. |
| 122^{c} | 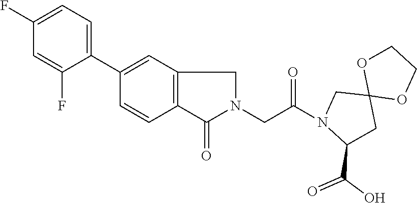 [SMILES: O=C(O)[C@@H]1CC2(CN1C(=O)CN1Cc3cc(-c4ccc(F)cc4F)ccc3C1=O)OCCO2] | 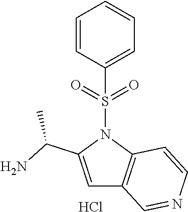 | ¹H NMR (400 MHz, DMSO-d6): δ 10.99 (s, 1H), 8.71 (t, J = 13.5 Hz, 1H), 8.62 (d, J = 15.2 Hz, 2H), 8.43 (s, 1H), 8.07 (d, J = 5.4 Hz, 1H), 7.84 (d, J = 8.3 Hz, 1H), 7.78 (s, 1H), 7.76-7.61 (m, 1H), 7.43 (d, J = 11.0 Hz, 2H), 7.26 (q, J = 9.8 Hz, 2H), 6.73 (s, 1H), 6.40 (s, 1H), 5.14 (t, J = 7.5 Hz, 1H), 4.62 (s, 1H), 4.57 (d, J = 2.8 Hz, 2H), 4.54-4.44 (m, 1H), 4.39 (d, J = 15.3 Hz, 2H), 4.10-3.87 (m, 2H), 3.85 (d, J = 11.0 Hz, 1H), 3.67 (d, J = 10.8 Hz, 2H), 2.32 (t, J = 10.6 Hz, 1H), 2.22-2.02 (m, 1H), 1.55 (d, J = 6.9 Hz, 3H). LC/MS (ESI) m/z: 602 (M + H)⁺. RT (Method A): 1.42 min. |
| 123^{c} | 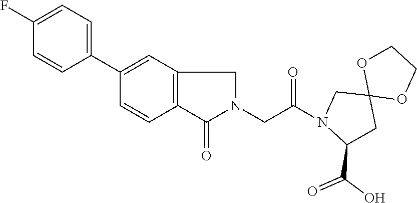 [SMILES: O=C(O)[C@@H]1CC2(CN1C(=O)CN1Cc3cc(-c4ccc(F)cc4)ccc3C1=O)OCCO2] | 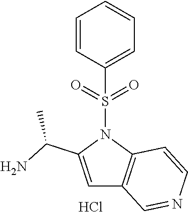 | ¹H NMR (400 MHz, DMSO-d6): δ 10.94 (s, 1H), 8.71 (d, J = 9.1 Hz, 1H), 8.63 (d, J = 6.7 Hz, 1H), 8.24 (s, 1H), 8.00 (d, J = 4.8 Hz, 1H), 7.83 (s, 1H), 7.73 (ddd, J = 16.0, 9.8, 5.3 Hz, 2H), 7.28 (t, J = 8.6 Hz, 3H), 6.63 (d, J = 5.6 Hz, 1H), 6.28 (s, 1H), 5.07 (t, J = 7.5 Hz, 1H), 4.55 (d, J = 7.9 Hz, 1H), 4.42 (d, J = 17.6 Hz, 1H), 4.32 (q, J = 7.0 Hz, 2H), 4.03-3.83 (m, 6H), 3.82 (s, 1H), 3.60 (d, J = 10.8 Hz, 2H), 2.24 (dt, J = 14.5, 7.3 Hz, 1H), 2.12-1.96 (m, 1H), 1.48 (d, J = 6.9 Hz, 3H). LC/MS (ESI) m/z: 584 (M + H)⁺. RT (Method A): 1.38 min. |
| 126^{c,d} | 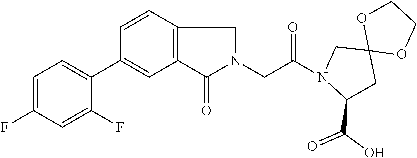 [SMILES: O=C(O)[C@@H]1CC2(CN1C(=O)CN1Cc3ccc(-c4ccc(F)cc4F)cc3C1=O)OCCO2] | 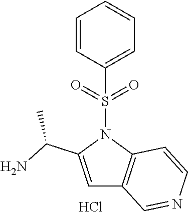 | ¹H NMR (400 MHz, DMSO-d6): δ 11.09 (s, 1H), 8.58 (d, J = 5.6 Hz, 1H), 8.23 (s, 1H), 7.91 (d, J = 5.6 Hz, 1H), 7.82-7.70 (m, 1H), 7.67 (d, J = 12.5 Hz, 1H), 7.37 (tt, J = 9.2, 2.6 Hz, 1H), 7.19 (td, J = 8.4, 2.7 Hz, 1H), 6.99 (d, J = 5.6 Hz, 1H), 6.32 (s, 1H), 4.66-4.12 (m, 5H), 4.00-3.81 (m, 3H), 3.77 (d, J = 10.8 Hz, 1H), 3.59 (d, J = 10.8 Hz, 1H), 2.28 (dd, J = 13.0, 8.4 Hz, 1H), 2.04 (dd, J = 13.0, 7.4 Hz, 1H). LC/MS (ESI) m/z: 602 (M + H)⁺. RT (Method A): 1.41 min. |
| 130^{a,d} | 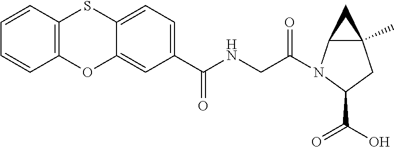 | 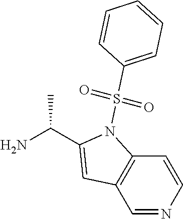 | ¹H NMR (400 MHz, CD₃OD): δ 8.64 (s, 1H), 7.92 (d, J = 5.6 Hz, 1H), 7.56 (d, J = 7.4 Hz, 2H), 7.25-7.12 (m, 4H), 7.10-7.01 (m, 2H), 6.50 (s, 1H), 5.24 (q, J = 6.8 Hz, 1H), 4.80 (d, J = 3.4 Hz, 1H), 4.50 (d, J = 16.6 Hz, 1H), 4.24 (d, J = 16.6 Hz, 1H), 3.37 (d, J = 5.0 Hz, 1H), 2.43 (t, J = 12.4 Hz, 1H), 2.16 (dd, J = 13.1, 3.7 Hz, 1H), 1.58 (d, J = 6.9 Hz, 3H), 1.31 (s, 3H), 1.28 (d, J = 7.4 Hz, 1H), 0.83 (t, J = 5.4 Hz, 1H). LC/MS (ESI) m/z: 568 (M + H)⁺. RT (Method A): 1.54 min. |
| 140^{c,d} | 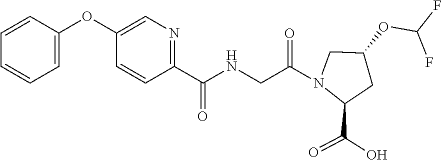 [SMILES: O=C(NCC(=O)N1C[C@H](OC(F)F)C[C@H]1C(=O)O)c1ccc(Oc2ccccc2)cn1] | 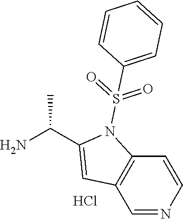 | ¹H NMR (400 MHz, DMSO-d6): δ 11.09 (s, 1H), 8.90 (d, J = 8.0 Hz, 1H), 8.72 (t, J = 5.4 Hz, 1H), 8.68 (d, J = 8.5 Hz, 1H), 8.63 (d, J = 7.9 Hz, 2H), 8.45-8.32 (m, 1H), 8.11-7.95 (m, 2H), 7.88 (d, J = 5.6 Hz, 1H), 7.56-7.36 (m, 4H), 7.22 (t, J = 7.1 Hz, 2H), 7.13 (d, J = 7.8 Hz, 3H), 7.06 (d, J = 5.6 Hz, 1H), 6.94 (s, 1H), 6.73 (d, J = 16.5 Hz, 1H), 6.45 (s, 1H), 6.18 (s, 1H), 5.10 (dt, J = 14.7, 7.0 Hz, 1H), 4.93 (s, 1H), 4.59 (t, J = 7.2 Hz, 0H), 4.40 (t, J = 7.7 Hz, 1H), 4.19 (dd, J = 17.3, 5.3 Hz, 1H), 4.09 (dd, J = 17.1, 5.6 Hz, 1H), 3.82 (dd, J = 11.6, 4.4 Hz, 1H), 3.73 (q, J = 14.4 Hz, 1H), 2.26 (t, J = 10.3 Hz, 2H), 2.11 (ddd, J = 13.2, 8.1, 4.8 Hz, 1H), 1.42 (d, J = 6.9 Hz, 3H). LC/MS (ESI) m/z: 579 (M + H)⁺. RT (Method A): 1.49 min. |
| 143^{a,d} | 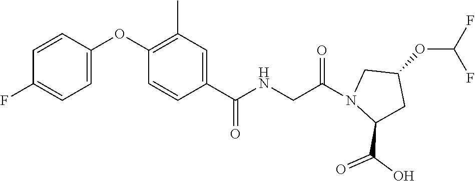 | 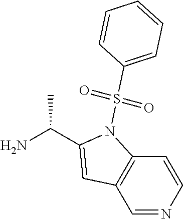 | ¹H NMR (400 MHz, CD₃OD): δ 8.65 (s, 1H), 7.87 (s, 2H), 7.72 (d, J = 8.2 Hz, 1H), 7.18-7.00 (m, 5H), 6.82 (d, J = 8.5 Hz, 1H), 6.72-6.34 (m, 2H), 5.30 (q, J = 6.7 Hz, 1H), 5.06 (s, 1H), 4.57 (t, J = 7.8 Hz, 1H), 4.35 (d, J = 16.8 Hz, 1H), 4.16 (d, J = 16.7 Hz, 1H), 3.93 (dd, J = 29.7, 10.5 Hz, 2H), 2.53-2.41 (m, 1H), 2.34 (s, 3H), 2.28 (d, J = 7.8 Hz, 1H), 1.61 (d, J = 6.8 Hz, 3H). LC/MS (ESI) m/z: 610 (M + H)⁺. RT (Method A): 1.95 min. |
| 147^{a} | 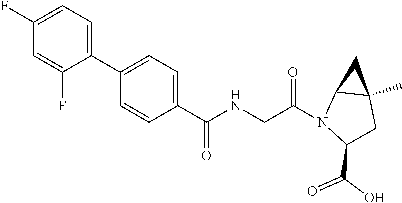 | 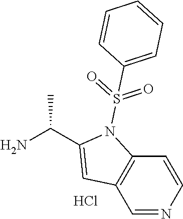 | ¹H NMR (400 MHz, CD₃OD): δ 8.65 (s, 1H), 7.96 (d, J = 7.6 Hz, 2H), 7.90 (d, J = 5.8 Hz, 1H), 7.62 (d, J = 8.1 Hz, 2H), 7.59-7.55 (m, 1H), 7.11 (dd, J = 14.0, 6.4 Hz, 3H), 6.51 (s, 1H), 5.25 (q, J = 6.6 Hz, 1H), 4.53 (d, J = 16.4 Hz, 1H), 4.29 (d, J = 16.6 Hz, 1H), 3.39 (d, J = 5.7 Hz, 1H), 2.43 (t, J = 12.2 Hz, 1H), 2.17 (d, J = 13.7 Hz, 1H), 1.59 (d, J = 6.8 Hz, 3H), 1.32 (s, 4H), 0.82 (t, J = 5.3 Hz, 1H). LC/MS (ESI) m/z: 558 (M + H)⁺. RT (Method A): 3.26 min. |
| 149^{a} | 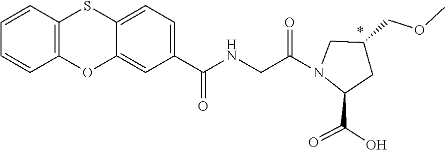 | 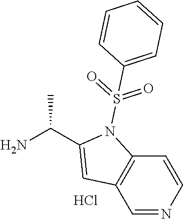 | ¹H NMR (400 MHz, DMSO-d6): δ 11.13 (s, 1H), 8.81 (s, 1H), 8.69-8.41 (m, 2H), 7.96 (d, J = 5.3 Hz, 1H), 7.63 (d, J = 9.5 Hz, 2H), 7.39 (t, J = 8.1 Hz, 1H), 7.27 (dd, J = 20.3, 6.9 Hz, 2H), 7.11 (dd, J = 13.5, 6.4 Hz, 3H), 6.39 (s, 1H), 5.19-5.10 (m, 1H), 4.40 (d, J = 7.8 Hz, 1H), 4.10 (ddd, J = 47.0, 17.5, 6.1 Hz, 2H), 3.80 (t, J = 8.6 Hz, 1H), 3.39-3.34 (m, 2H), 3.26 (d, J = 12.1 Hz, 4H), 2.69 (dd, J = 14.8, 7.1 Hz, 1H), 1.93 (dd, J = 20.6, 9.8 Hz, 2H), 1.46 (d, J = 6.8 Hz, 3H). LC/MS (ESI) m/z: 586 (M + H)⁺. RT (Method A): 1.84 min. |
| 162^{a} | 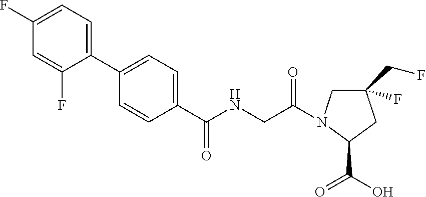 [SMILES: O=C(NCC(=O)N1C[C@@](F)(CF)C[C@H]1C(=O)O)c1ccc(-c2ccc(F)cc2F)cc1] | 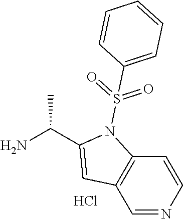 | ¹H NMR (400 MHz, CD₃OD): δ 8.67 (s, 1H), 8.01 (d, J = 7.8 Hz, 2H), 7.85 (d, J = 5.6 Hz, 1H), 7.67 (d, J = 7.9 Hz, 2H), 7.60 (d, J = 8.5 Hz, 1H), 7.10 (d, J = 8.9 Hz, 3H), 6.57 (s, 1H), 5.31 (q, J = 7.1 Hz, 1H), 4.67 (dd, J = 18.1, 10.2 Hz, 2H), 4.41 (d, J = 16.7 Hz, 1H), 4.19 (d, J = 15.9 Hz, 1H), 4.10-3.98 (m, 1H), 2.65-2.53 (m, 1H), 2.35-2.20 (m, 1H), 1.61 (d, J = 6.7 Hz, 3H). LC/MS (ESI) m/z: 582 (M + H)⁺. RT (Method A): 1.84 min. |
| 165^{a,d} | 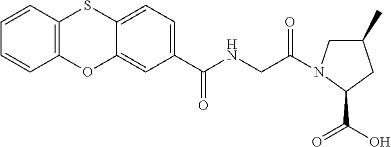 | 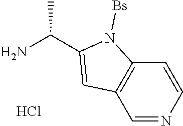 | ¹H NMR (400 MHz, CD₃OD): δ 8.63 (s, 1H), 7.87 (d, J = 5.6 Hz, 1H), 7.64- 7.51 (m, 2H), 7.25 (d, J = 8.0 Hz, 1H), 7.21-7.14 (m, 2H), 7.08 (t, J = 6.0 Hz, 2H), 7.02 (d, J = 7.9 Hz, 1H), 6.49 (s, 1H), 5.31 (d, J = 6.8 Hz, 1H), 4.38 (d, J = 15.7 Hz, 2H), 4.08 (d, J = 16.8 Hz, 1H), 3.92 (t, J = 8.0 Hz, 1H), 3.20 (t, J = 9.7 Hz, 1H), 2.39 (d, J = 6.6 Hz, 2H), 1.61 (t, J = 8.0 Hz, 4H), 1.15 (d, J = 5.0 Hz, 3H). LC/MS (ESI) m/z: 556 (M + H)⁺. RT (Method A): 1.56 min. |
| 166^{a,d} | 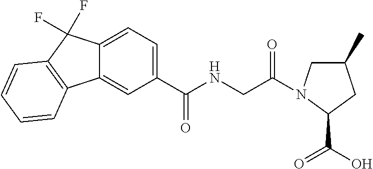 | 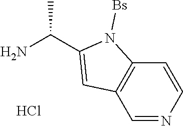 | ¹H NMR (400 MHz, CD₃OD): δ 8.86 (s, 1H), 8.39 (s, 1H), 8.23 (s, 1H), 7.96- 7.82 (m, 2H), 7.69 (d, J = 21.1 Hz, 3H), 7.57 (s, 1H), 7.46 (s, 2H), 6.85 (s, 1H), 5.34 (d, J = 6.4 Hz, 1H), 4.43 (d, J = 9.8 Hz, 2H), 4.18 (d, J = 16.8 Hz, 1H), 3.99 (s, 1H), 3.24 (d, J = 9.5 Hz, 1H), 2.46 (s, 2H), 1.64 (d, J = 6.9 Hz, 4H), 1.19 (d, J = 4.3 Hz, 3H). LC/MS (ESI) m/z: 558 (M + H)⁺. RT (Method A): 1.28 min. |
| 167^{a,d} | 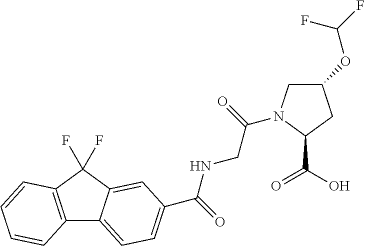 [SMILES: O=C(NCC(=O)N1C[C@H](OC(F)F)C[C@H]1C(=O)O)c1ccc2c(c1)C(F)(F)c1ccccc1-2] | 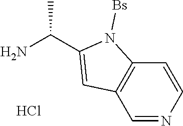 | ¹H NMR (400 MHz, CD₃OD): δ 8.90 (d, J = 22.1 Hz, 1H), 8.42 (s, 1H), 8.17 (s, 1H), 8.10 (d, J = 7.8 Hz, 1H), 7.97 (d, J = 5.4 Hz, 1H), 7.88-7.78 (m, 2H), 7.68 (d, J = 6.4 Hz, 1H), 7.60 (t, J = 7.0 Hz, 1H), 7.50 (dd, J = 17.6, 7.1 Hz, 2H), 6.89 (d, J = 27.3 Hz, 1H), 6.55 (t, J = 74.4 Hz, 1H), 5.33 (d, J = 6.1 Hz, 1H), 5.08 (s, 1H), 4.60 (t, J = 7.8 Hz, 1H), 4.36 (d, J = 16.5 Hz, 1H), 4.22 (d, J = 16.5 Hz, 1H), 3.97 (dd, J = 30.0, 11.3 Hz, 2H), 2.69-2.45 (m, 1H), 2.29 (s, 1H), 1.67 (dd, J = 23.3, 6.6 Hz, 3H). LC/MS (ESI) m/z: 610 (M − H)⁺. RT (Method A): 1.45 min. |
| 171^{c,d} | 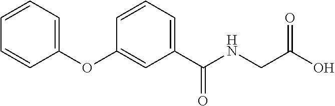 [SMILES: O=C(O)CNC(=O)c1cccc(Oc2ccccc2)c1] | 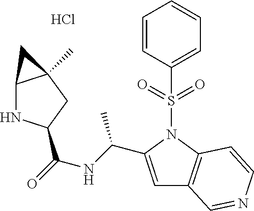 | ¹H NMR (400 MHz, DMSO-d6): δ 11.01 (s, 1H), 8.78 (t, J = 5.7 Hz, 1H), 8.59 (s, 1H), 8.40 (d, J = 8.4 Hz, 1H), 8.33 (s, 1H), 7.86 (d, J = 5.6 Hz, 1H), 7.63 (d, J = 7.7 Hz, 1H), 7.52-7.38 (m, 2H), 7.35-7.27 (m, 2H), 7.17 (dd, J = 8.1, 2.4 Hz, 1H), 7.08 (t, J = 7.4 Hz, 1H), 7.01-6.79 (m, 3H), 6.29 (s, 1H), 5.02 (q, J = 7.3 Hz, 1H), 4.64 (dd, J = 11.2, 3.6 Hz, 1H), 4.32 (dd, J = 16.6, 5.8 Hz, 1H), 3.98 (dd, J = 16.6, 5.8 Hz, 1H), 2.33-2.12 (m, 1H), 1.96 (dd, J = 13.2, 3.6 Hz, 1H), 1.37 (d, J = 7.0 Hz, 3H), 1.25 (dd, J = 5.1, 2.4 Hz, 1H), 1.16 (s, 3H). LC/MS (ESI) m/z: 538 (M + H)⁺. RT (Method A): 1.55 min. |
| 172^{c,d} | 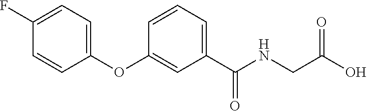 [SMILES: O=C(O)CNC(=O)c1cccc(Oc2ccc(F)cc2)c1] | 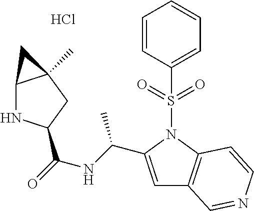 | ¹H NMR (400 MHz, DMSO-d6): δ 11.00 (s, 1H), 8.78 (t, J = 5.8 Hz, 1H), 8.59 (s, 1H), 8.38 (d, J = 8.4 Hz, 1H), 8.31 (s, 1H), 7.85 (d, J = 5.6 Hz, 1H), 7.62 (d, J = 7.7 Hz, 1H), 7.50-7.37 (m, 2H), 7.22-7.09 (m, 4H), 7.08-6.98 (m, 2H), 6.96 (d, J = 5.6 Hz, 1H), 6.29 (s, 1H), 5.03 (dp, J = 14.6, 7.2 Hz, 1H), 4.64 (dd, J = 11.2, 3.6 Hz, 1H), 4.31 (dd, J = 16.6, 5.9 Hz, 1H), 3.99 (dd, J = 16.6, 5.8 Hz, 1H), 2.35-2.17 (m, 1H), 1.96 (dd, J = 13.2, 3.6 Hz, 1H), 1.37 (d, J = 6.9 Hz, 3H), 1.25 (dd, J = 5.0, 2.4 Hz, 1H), 1.18 (s, 3H). LC/MS (ESI) m/z: 556 (M + H)⁺. RT (Method A): 1.55 min. |
| 173^{a} | 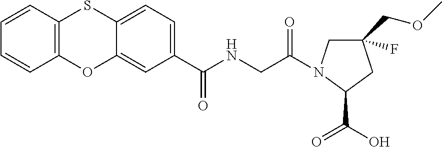 | 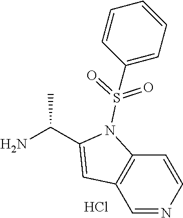 | ¹H NMR (400 MHz, CD₃OD): δ 8.85 (s. 1H), 8.43 (s, 1H), 7.98 (d, J = 5.7 Hz, 1H), 7.55 (d, J = 18.0 Hz, 2H), 7.44 (s, 1H), 7.26-7.15 (m, 3H), 7.10 (d, J = 7.3 Hz, 1H), 7.02 (d, J = 7.2 Hz, 1H), 6.83 (s, 1H), 5.33 (d, J = 5.6 Hz, 1H), 4.62 (t, J = 8.2 Hz, 1H), 4.33 (d, J = 16.8 Hz, 1H), 4.14 (d, J = 17.2 Hz, 1H), 3.96 (d, J = 35.7 Hz, 2H), 3.77-3.70 (m, 2H), 3.45 (s, 3H), 2.55 (s, 1H), 2.25 (dd, J = 26.5, 11.5 Hz, 1H), 1.63 (d, J = 6.1 Hz, 3H). LC/MS (ESI) m/z: 604 (M + H)⁺. RT (Method A): 1.40 min. |
| 175^{a} | 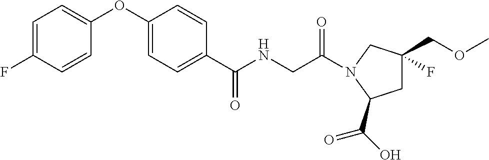 | 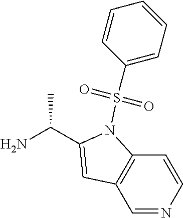 | ¹H NMR (400 MHz, CD₃OD): δ 8.67 (d, J = 16.6 Hz, 1H), 7.98 (t, J = 21.4 Hz, 1H), 7.54 (dd, J = 23.9, 15.7 Hz, 2H), 7.26-7.14 (m, 4H), 7.06 (dd, J = 21.6, 7.4 Hz, 2H), 6.56 (d, J = 26.2 Hz, 1H), 5.28 (d, J = 7.1 Hz, 1H), 4.63 (s, 1H), 4.16 (ddd, J = 31.9, 26.5, 13.1 Hz, 3H), 3.88 (d, J = 6.8 Hz, 1H), 3.46 (d, J = 7.8 Hz, 1H), 2.40 (dd, J = 25.8, 18.9 Hz, 2H), 1.63 (dd, J = 22.9, 6.3 Hz, 3H). LC/MS (ESI) m/z: 592 (M + H)⁺. RT (Method A): 1.34 min. |
| 200^{a} | 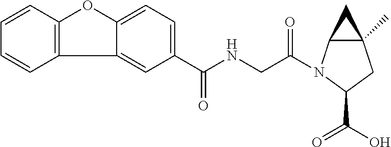 | 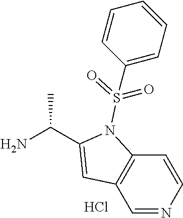 | ¹H NMR (400 MHz, CD₃OD): δ 8.63 (d, J = 9.7 Hz, 2H), 8.04 (t, J = 7.5 Hz, 2H), 7.76 (d, J = 5.9 Hz, 1H), 7.69-7.63 (m, 2H), 7.55 (t, J = 7.8 Hz, 1H), 7.41 (t, J = 7.5 Hz, 1H), 7.12 (d, J = 5.7 Hz, 1H), 6.51 (s, 1H), 5.25 (q, J = 6.8 Hz, 1H), 4.58 (d, J = 16.6 Hz, 1H), 4.33 (d, J = 16.6 Hz, 1H), 3.41 (s, 1H), 2.44 (t, J = 12.5 Hz, 1H), 2.18 (dd, J = 13.5, 4.1 Hz, 1H), 1.59 (d, J = 6.9 Hz, 3H), 1.37 (dd, J = 5.5, 2.4 Hz, 1H), 1.33 (s, 3H), 1.28 (s, 1H), 0.85 (t, J = 5.5 Hz, 1H). LC/MS (ESI) m/z: 536 (M + H)⁺. RT (Method A): 1.46 min. |
| 219^{a} | 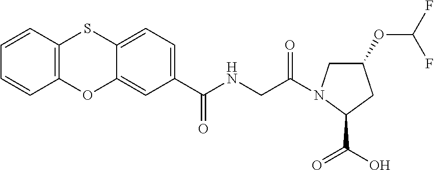 [SMILES: O=C(NCC(=O)N1C[C@H](OC(F)F)C[C@H]1C(=O)O)c1ccc2c(c1)Oc1ccccc1S2] | 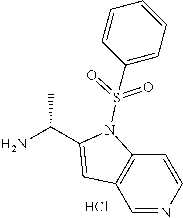 | ¹H NMR (400 MHz, CD₃OD): δ 8.66 (s, 1H), 7.90 (d, J = 5.9 Hz, 1H), 7.62- 7.53 (m, 2H), 7.26 (d, J = 8.0 Hz, 1H), 7.21-7.07 (m, 4H), 7.03 (d, J = 8.1 Hz, 1H), 6.72-6.35 (m, 2H), 5.31 (q, J = 6.4 Hz, 1H), 5.12-5.02 (m, 1H), 4.57 (t, J = 8.0 Hz, 1H), 4.34 (d, J = 16.8 Hz, 1H), 4.15 (d, J = 16.8 Hz, 1H), 3.99-3.93 (m, 1H), 3.89 (d, J = 11.5 Hz, 1H), 2.52-2.44 (m, 1H), 2.31-2.23 (m, 1H), 1.61 (d, J = 7.0 Hz, 3H). LC/MS (ESI) m/z: 608 (M + H)⁺. RT (Method A): 1.50 min. |
| 220^{a} | 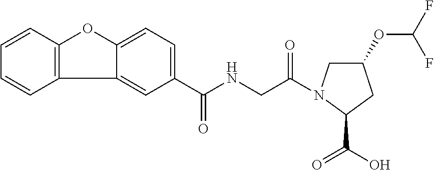 [SMILES: O=C(NCC(=O)N1C[C@H](OC(F)F)C[C@H]1C(=O)O)c1ccc2oc3ccccc3c2c1] | 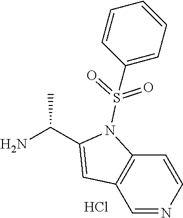 | ¹H NMR (400 MHz, CD₃OD): δ 8.81 (d, J = 11.0 Hz, 1H), 8.64-8.58 (m, 1H), 8.44 (s, 1H), 8.05 (d, J = 8.4 Hz, 2H), 7.82 (d, J = 6.4 Hz, 1H), 7.71- 7.63 (m, 2H), 7.56 (s, 1H), 7.43 (d, J = 7.2 Hz, 2H), 6.83 (d, J = 3.4 Hz, 1H), 6.65 (d, J = 74.5 Hz, 1H), 5.33 (d, J = 7.0 Hz, 1H), 5.09 (s, 1H), 4.62 (t, J = 8.0 Hz, 1H), 4.37 (d, J = 5.0 Hz, 1H), 4.28-4.22 (m, 1H), 4.01 (d, J = 4.3 Hz, 1H), 3.96 (s, 1H), 2.55-2.48 (m, 1H), 2.34-2.27 (m, 1H), 1.63 (d, J = 7.0, 3H). LC/MS (ESI) m/z: 576 (M + H)⁺. RT (Method A): 1.35 min. |
| 238^{a} | 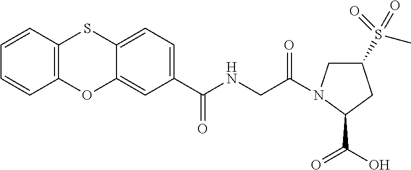 | 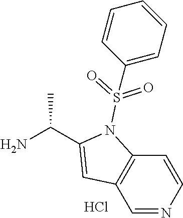 | ¹H NMR (400 MHz, CD₃OD): δ 8.65 (s, 1H), 7.93 (d, J = 5.8 Hz, 1H), 7.59- 7.53 (m, 2H), 7.26-7.15 (m, 4H), 7.10-7.02 (m, 2H), 6.52 (s, 1H), 5.28 (dd, J = 13.8, 6.8 Hz, 1H), 4.69 (dd, J = 8.4, 5.6 Hz, 1H), 4.31-4.13 (m, 5H), 3.09 (s, 3H), 2.81-2.73 (m, 1H), 2.50-2.42 (m, 1H), 1.60 (d, J = 7.0 Hz, 3H). LC/MS (ESI) m/z: 620 (M + H)⁺. RT (Method A): 1.30 min. |
| 300^{a} | 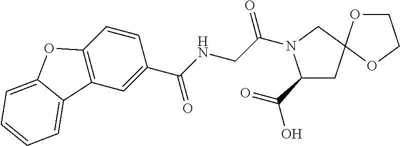 [SMILES: O=C(NCC(=O)N1CC2(C[C@H]1C(=O)O)OCCO2)c1ccc2oc3ccccc3c2c1] | 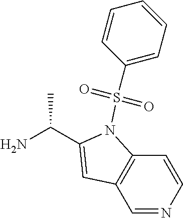 | ¹H-NMR (400 MHz, CD₃OD): δ 8.74 (d, J = 9.6 Hz, 1H), 8.59 (d, J = 8.7 Hz, 1H), 8.06-7.99 (m, 2H), 7.83 (d, J = 5.8 Hz, 1H), 7.68-7.62 (m, 2H), 7.57- 7.52 (m, 1H), 7.43-7.39 (m, 1H), 7.35 (d, J = 5.6 Hz, 1H), 6.73 (d, J = 5.2 Hz, 1H), 5.35-5.28 (m, 1H), 4.58 (t, J = 8.0 Hz, 1H), 4.33 (d, J = 16.8 Hz, 1H), 4.21 (d, J = 16.7 Hz, 1H), 4.08-4.00 (m, 4H), 3.84 (s, 2H), 2.46-2.40 (m, 1H), 2.31-2.25 (m, 1H), 1.63-1.57 (m, 3H). LC/MS (ESI) m/z: 568(M + H)⁺. RT (Method A): 1.19 min. |

^{a}HATU was used in place of PyBOP.
^{b}HBTU was used in place of PyBOP.
^{c}T₃P was used in place of PyBOP.
^{d}The product from the coupling reaction was used without further purification.

### Scheme 9. Synthesis of (2S,4R)-N-((1H-pyrrolo[3,2-c]pyridin-2-yl)methyl)-4-fluoro-4-(methoxymethyl)-1-((phenoxathiine-3-carbonyl)glycyl)pyrrolidine-2-carboxamide (Compound 25)

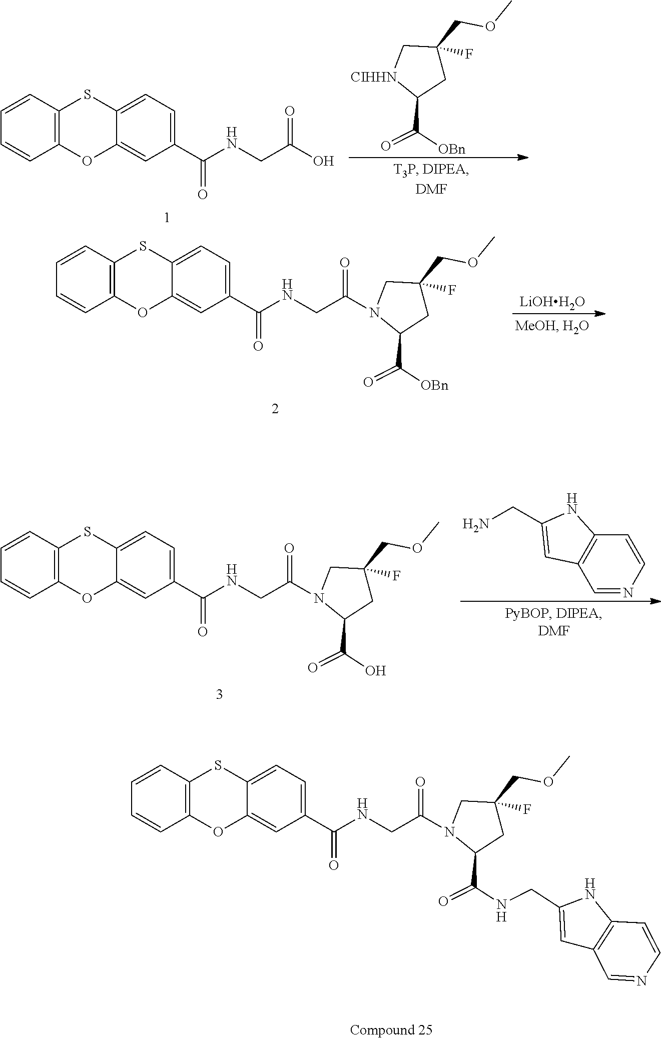

### Step 1: benzyl (2S,4R)-4-fluoro-4-(methoxymethyl)-1-((phenoxathiine-3-carbonyl) glycyl)pyrrolidine-2-carboxylate

To a mixture of (phenoxathiine-3-carbonyl)glycine (45 mg, 0.15 mmol) and benzyl (2S,4R)-4-fluoro-4-(methoxymethyl)pyrrolidine-2-carboxylate hydrochloride (58.9 mg, 0.19 mmol) in DMF (3 mL) was added DIPEA (115.8 mg, 0.9 mmol) and T₃P (285 mg, 0.45 mmol, 50% in EtOAc wt.) under N₂ atmosphere at 0° C. and the mixture was stirred at room temperature for 2 hours. The mixture was quenched with saturated aq. NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-58% EtOAc in PE) to give benzyl (2S,4R)-4-fluoro-4-(methoxymethyl)-1-((phenoxathiine-3-carbonyl)glycyl)pyrrolidine-2-carboxylate (75 mg, yield 91.2%) as a white solid. LC/MS (ESI) (m/z): 551 (M+H)⁺.

### Step 2: (2S,4R)-4-fluoro-4-(methoxymethyl)-1-((phenoxathiine-3-carbonyl)glycyl) pyrrolidine-2-carboxylic acid

To a solution of benzyl (2S,4R)-4-fluoro-4-(methoxymethyl)-1-((phenoxathiine-3-carbonyl)glycyl)pyrrolidine-2-carboxylate (75 mg, 0.16 mmol) in MeOH/H₂O (4 mL, v/v=3/1) was added LiOH·H₂O (17.2 mg, 0.48 mmol) and the mixture was stirred at room temperature for 2 hours. The mixture was acidified with 1N aq. HCl to pH-3 and extracted with EtOAc twice. The combined organic layers were washed with brine, dried over anhydrous Na₂SO₄, filtered, and concentrated under reduced pressure to dryness to give (2S,4R)-4-fluoro-4-(methoxymethyl)-1-((phenoxathiine-3-carbonyl) glycyl)pyrrolidine-2-carboxylic acid (58 mg, yield 92.4%) as a yellow solid, which was used directly in the next step without further purification. LC/MS (ESI) m/z: 461 (M+H)⁺.

### Step 3: (2S,4R)-N-((1H-pyrrolo[3,2-c]pyridin-2-yl)methyl)-4-fluoro-4-(methoxymethyl)-1-((phenoxathiine-3-carbonyl)glycyl)pyrrolidine-2-carboxamide (Compound 25)

To a mixture of (2S,4R)-4-fluoro-4-(methoxymethyl)-1-((phenoxathiine-3-carbonyl)glycyl)pyrrolidine-2-carboxylic acid (58 mg, 0.13 mmol) and (1H-pyrrolo[3,2-c]pyridin-2-yl)methanamine (20.2 mg, 0.14 mmol) in DMF (3 mL) was added DIPEA (97.6 mg, 0.78 mmol) and PyBOP (65.5 mg, 0.13 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=12:1) and further purified by prep-HPLC to give Compound 25 (18 mg, yield 24.2%) as a white solid. ¹H NMR (400 MHz, CD₃OD): δ 8.83 (s, 1H), 9.06 (s, 1H), 7.58 (d, J=6.5 Hz, 1H), 7.52-7.48 (m, 7.3 (d, J=1.8 Hz, 1), 7.22-7.16 (m, 3H), 7.11 (d, J=7.5 Hz, 1H), 7.02 (d, J=8.1 Hz, 1H), 6.86 (s, 1H), 4.70 (d, J=8.6 Hz, 2H), 4.64 (d, J=9.6 Hz, 1H), 4.21 (s, 2H), 4.06 (d, J=16.8 Hz, 2H), 3.75 (d, J=4.3 Hz, 1H), 3.71 (d, J=7.1 Hz, 1H), 3.45 (s, 3H), 2.59 (dd, J=14.7, 7.0 Hz, 1H), 2.31-2.20 (m, 1H). LC/MS (ESI) m/z: 590 (M+H)⁺. RT (Method A): 1.420 min.

The following compounds were prepared according to Scheme 9:

| Cmpd # | Reactant A | Reactant B | Reactant C | Characterization Data |
|---|---|---|---|---|
| 26 | 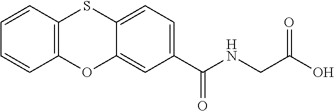 [SMILES: O=C(O)CNC(=O)c1ccc2c(c1)Oc1ccccc1S2] | 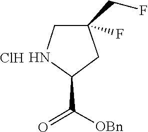 [SMILES: Cl ; O=C(OCc1ccccc1)[C@@H]1C[C@](F)(CF)CN1] | 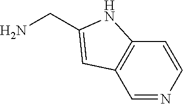 [SMILES: NCc1cc2cnccc2[nH]1] | ¹H NMR (400 MHz, CD₃OD): δ 8.80 (s, 1H), 8.41 (s, 1H), 8.05 (d, J = 6.5 Hz, 1H), 7.54 (d, J = 6.5 Hz, 1H), 7.49 (dd, J = 8.1, 1.7 Hz, 1H), 7.38 (d, J = 1.6 Hz, 1H), 7.20 (d, J = 7.2 Hz, 1H), 7.15 (d, J = 5.7 Hz, 1H), 7.11-7.07 (m, 1H), 7.01 (d, J = 8.1 Hz, 1H), 6.82 (s, 1H), 4.82-4.76 (m, 1H), 4.72-4.69 (m, 2H), 4.65 (t, J = 10.3 Hz, 2H), 4.22 (s, 2H), 4.11 (s, 1H), 4.01 (t, J = 16.3 Hz, 1H), 2.65 (dd, J = 14.9, 7.0 Hz, 1H), 2.33-2.20 (m, 1H). LC/MS (ESI) m/z: 578 (M + H)⁺. RT (Method A): 1.42 min. |
| 27 | 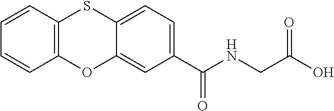 [SMILES: O=C(O)CNC(=O)c1ccc2c(c1)Oc1ccccc1S2] | 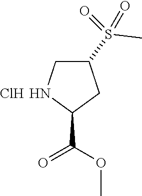 | 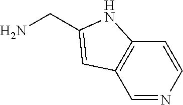 [SMILES: NCc1cc2cnccc2[nH]1] | ¹H NMR (400 MHz, CD₃OD): δ 8.96-8.72 (m, 1H), 8.46 (s, 1H), 8.14 (dd, J = 52.1, 6.1 Hz, 1H), 7.65 (dd, J = 62.2, 5.9 Hz, 1H), 7.51- 7.30 (m, 2H), 7.24-7.04 (m, 4H), 7.00 (t, J = 9.1 Hz, 1H), 6.84 (d, J = 41.7 Hz, 1H), 4.78-4.68 (m, 2H), 4.67 (s, 1H), 4.21 (ddd, J = 51.3, 44.4, 25.7 Hz, 5H), 3.07 (d, J = 25.8 Hz, 3H), 2.94-2.76 (m, 1H), 2.64-2.43 (m, 1H). LC/MS (ESI) m/z: 606 (M + H)⁺. RT (Method A): 1.29 min. |
| 30 | 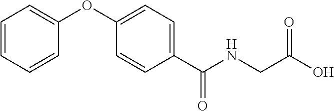 [SMILES: O=C(O)CNC(=O)c1ccc(Oc2ccccc2)cc1] | 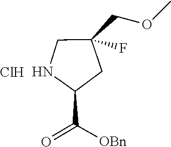 | 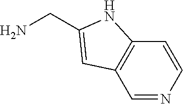 [SMILES: NCc1cc2cnccc2[nH]1] | ¹H NMR (400 MHz, CD₃OD): δ 8.86 (s, 1H), 8.43 (s, 1H), 8.10 (d, J = 6.4 Hz, 1H), 7.83 (d, J = 8.7 Hz, 2H), 7.57 (d, J = 6.4 Hz, 1H), 7.43 (t, J = 8.0 Hz, 2H), 7.21 (t, J = 7.4 Hz, 1H), 7.07 (d, J = 7.7 Hz, 2H), 7.01-6.96 (m, 2H), 6.83 (s, 1H), 4.74 (d, J = 16.4 Hz, 1H), 4.63 (dd, J = 17.2, 10.1 Hz, 2H), 4.22 (q, J = 16.7 Hz, 2H), 4.13- 4.02 (m, 1H), 4.01-3.89 (m, 1H), 3.75 (t, J = 7.5 Hz, 1H), 3.68 (t, J = 8.8 Hz, 1H), 3.42 (d, J = 15.1 Hz, 3H), 2.57 (td, J = 14.6, 7.7 Hz, 1H), 2.25 (ddd, J = 37.2, 14.1, 9.9 Hz, 1H). LC/MS (ESI) m/z: 560 (M + H)⁺. RT (Method A): 1.29 min. |
| 32 | 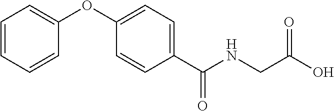 [SMILES: O=C(O)CNC(=O)c1ccc(Oc2ccccc2)cc1] | 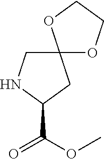 | 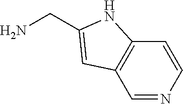 [SMILES: NCc1cc2cnccc2[nH]1] | ¹H NMR (400 MHz, CD₃OD): δ 8.91 (d, J = 35.3 Hz, 1H), 8.45 (s, 1H), 8.18 (dd, J = 32.7, 6.5 Hz, 1H), 7.86-7.77 (m, 2H), 7.62 (d, J = 6.5 Hz, 1H), 7.45-7.40 (m, 2H), 7.22 (t, J = 7.4 Hz, 1H), 7.09-7.05 (m, 2H), 7.00-6.91 (m, 2H), 6.83 (s, 1H), 4.71-4.67 (m, 2H), 4.63 (dd, J = 9.0, 6.0 Hz, 1H), 4.24 (d, J = 16.6 Hz, 1H), 4.11 (dd, J = 15.9, 12.3 Hz, 1H), 3.99 (d, J = 27.5 Hz, 4H), 3.87-3.76 (m, 2H), 2.48 (dd, J = 13.1, 9.1 Hz, 1H), 2.28 (dd, J = 13.1, 5.9 Hz, 1H). LC/MS (ESI) (m/z): 556 (M + H)⁺. RT (Method A): 1.19 min. |
| 35 | 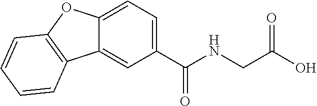 [SMILES: O=C(O)CNC(=O)c1ccc2oc3ccccc3c2c1] | 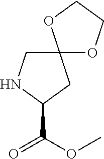 | 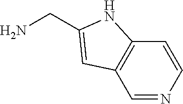 [SMILES: NCc1cc2cnccc2[nH]1] | ¹H NMR (400 MHz, CD₃OD): δ 8.69 (s, 1H), 8.45 (s, 1H), 8.01 (d, J = 7.7 Hz, 1H), 7.96-7.91 (m, 2H), 7.65 (d, J = 8.3 Hz, 1H), 7.61- 7.55 (m, 2H), 7.49 (d, J = 6.4 Hz, 1H), 7.42 (d, J = 7.4 Hz, 1H), 6.80 (s, 1H), 4.73 (s, 1H), 4.66 (dd, J = 9.8, 6.9 Hz, 2H), 4.32 (d, J = 16.4 Hz, 1H), 4.18-4.13 (m, 1H), 4.04 (s, 4H), 3.95-3.86 (m, 2H), 2.50 (dd, J = 12.9, 9.1 Hz, 1H), 2.30 (dd, J = 13.2, 5.7 Hz, 1H). LC/MS (ESI) m/z: 554 (M + H)⁺. RT (Method A): 1.16 min. |
| 137^{a} | 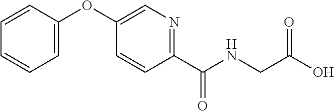 [SMILES: O=C(O)CNC(=O)c1ccc(Oc2ccccc2)cn1] | 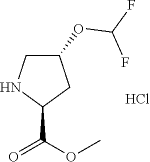 | 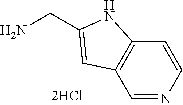 [SMILES: NCc1cc2cnccc2[nH]1] | ¹H NMR (400 MHz, DMSO-d6): δ 11.17 (s, 1H), 8.80-8.49 (m, 2H), 8.39 (d, J = 3.0 Hz, 1H), 8.24 (s, 1H), 8.12-7.83 (m, 3H), 7.55- 7.34 (m, 2H), 7.22 (q, J = 7.8 Hz, 2H), 7.16 (s, 1H), 6.93 (s, 1H), 6.74 (s, 1H), 6.32 (s, 1H), 5.01-4.79 (m, 1H), 4.60 (d, J = 7.5 Hz, 1H), 4.40 (tt, J = 15.6, 8.3 Hz, 2H), 4.12 (qt, J = 9.2, 5.1 Hz, 2H), 3.92-3.75 (m, 1H), 2.37-2.14 (m, 2H). LC/MS (ESI) m/z: 565 (M + H)⁺. RT (Method A): 1.45 min. |
| 179^{b,c} | 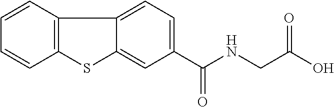 [SMILES: O=C(O)CNC(=O)c1ccc2c(c1)sc1ccccc12] | 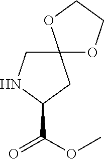 | 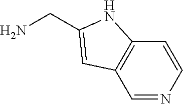 [SMILES: NCc1cc2cnccc2[nH]1] | ¹H NMR (400 MHz, CD₃OD): δ 8.57 (d, J = 10.0 Hz, 1H), 8.36 (t, J = 8.6 Hz, 1H), 8.32-8.29 (m, 1H), 8.26 (d, J = 8.3 Hz, 1H), 7.92 (ddd, J = 9.9, 7.1, 1.8 Hz, 2H), 7.85 (d, J = 5.9 Hz, 1H), 7.56-7.51 (m, 2H), 7.17 (d, J = 5.8 Hz, 1H), 6.56 (d, J = 31.4 Hz, 1H), 4.66- 4.58 (m, 3H), 4.25 (t, J = 11.1 Hz, 2H), 4.03 (t, J = 5.0 Hz, 4H), 3.83 (t, J = 7.4 Hz, 2H), 2.46 (dd, J = 13.1, 8.9 Hz, 1H), 2.29 (dd, J = 13.1, 6.2 Hz, 1H). LC/MS (ESI) m/z: 570 (M + H)⁺. RT (Method A): 1.23 min. |
| 187^{c} | 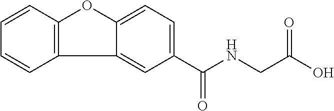 [SMILES: O=C(O)CNC(=O)c1ccc2oc3ccccc3c2c1] | 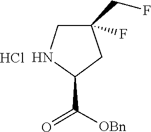 [SMILES: Cl ; O=C(OCc1ccccc1)[C@@H]1C[C@](F)(CF)CN1] | 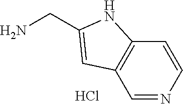 [SMILES: NCc1cc2cnccc2[nH]1 ; Cl] | ¹H NMR (400 MHz, CD₃OD): δ 8.56 (d, J = 1.4 Hz, 2H), 8.05-8.00 (m, 2H), 7.80 (d, J = 5.8 Hz, 1H), 7.64 (d, J = 8.5 Hz, 2H), 7.56- 7.52 (m, 1H), 7.44-7.39 (m, 1H), 7.13 (d, J = 5.8 Hz, 1H), 6.51 (d, J = 0.8 Hz, 1H), 4.78-4.65 (m, 4H), 4.55 (d, J = 16.5 Hz, 1H), 4.36 (d, J = 16.7 Hz, 1H), 4.24 (d, J = 16.6 Hz, 1H), 4.17 (dd, J = 18.9, 12.3 Hz, 1H), 4.09-3.97 (m, 1H), 2.63 (td, J = 14.9, 7.8 Hz, 1H), 2.28 (ddd, J = 35.7, 14.2, 9.5 Hz, 1H). LC/MS (ESI) m/z: 546 (M + H)⁺. RT (Method A): 1.22 min. |
| 203^{c} | 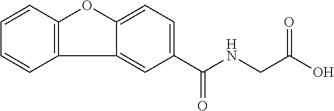 [SMILES: O=C(O)CNC(=O)c1ccc2oc3ccccc3c2c1] | 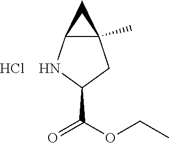 | 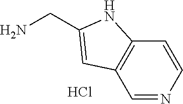 [SMILES: NCc1cc2cnccc2[nH]1 ; Cl] | ¹H NMR (400 MHz, CD₃OD): δ 8.58-8.48 (m, 2H), 7.97 (dd, J = 13.3, 6.7 Hz, 2H), 7.86 (dd, J = 5.8, 2.4 Hz, 1H), 7.65-7.52 (m, 3H), 7.40 (dd, J = 9.4, 5.6 Hz, 1H), 7.18 (d, J = 5.7 Hz, 1H), 6.50 (s, 1H), 4.89 (s, 1H), 4.66-4.36 (m, 4H), 3.44 (s, 1H), 2.45 (t, J = 12.1 Hz, 1H), 2.22 (d, J = 13.3 Hz, 1H), 1.32 (d, J = 3.4 Hz, 3H), 1.28 (d, J = 3.4 Hz, 1H), 0.85 (s, 1H). LC/MS (ESI) m/z: 522 (M + H)⁺. RT (Method A): 1.37 min. |
| 205^{c} | 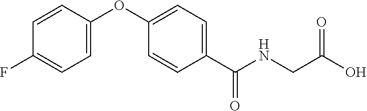 [SMILES: O=C(O)CNC(=O)c1ccc(Oc2ccc(F)cc2)cc1] | 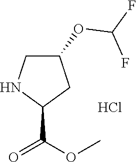 | 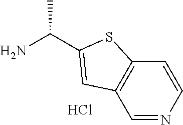 | ¹H NMR (400 MHz, CD₃OD): δ 8.95 (d, J = 9.7 Hz, 1H), 8.27 (t, J = 6.9 Hz, 1H), 7.89 (t, J = 5.2 Hz, 1H), 7.86-7.77 (m, 2H), 7.52 (s, 1H), 7.18-7.06 (m, 4H), 6.99-6.95 (m, 2H), 6.49 (td, J = 74.5, 15.0 Hz, 1H), 5.38 (dt, J = 20.6, 6.5 Hz, 1H), 4.98 (d, J = 33.4 Hz, 1H), 4.71-4.59 (m, 1H), 4.31-4.07 (m, 2H), 3.96-3.75 (m, 2H), 2.50 (ddd, J = 34.7, 30.8, 12.1 Hz, 1H), 2.41-2.22 (m, 1H), 1.66 (t, J = 13.0 Hz, 3H). LC/MS (ESI) m/z: 613 (M + H)⁺. RT (Method A): 1.55 min. |
| 211^{c} | 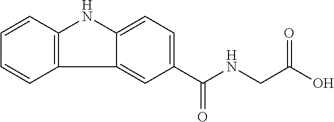 [SMILES: O=C(O)CNC(=O)c1ccc2[nH]c3ccccc3c2c1] | 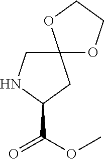 | 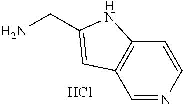 [SMILES: NCc1cc2cnccc2[nH]1 ; Cl] | ¹H NMR (400 MHz, CD₃OD): δ 8.62 (s, 1H), 8.54 (s, 1H), 8.06 (d, J = 7.7 Hz, 1H), 7.88 (dd, J = 8.5, 1.7 Hz, 1H), 7.80 (d, J = 5.8 Hz, 1H), 7.49 (d, J = 8.2 Hz, 1H), 7.45 (d, J = 4.2 Hz, 1H), 7.43 (d, J = 2.6 Hz, 1H), 7.22 (d, J = 7.2 Hz, 1H), 7.12 (d, J = 5.8 Hz, 1H), 6.51 (s, 1H), 4.70 (d, J = 16.1 Hz, 1H), 4.64-4.57 (m, 2H), 4.24 (d, J = 6.5 Hz, 2H), 4.02 (d, J = 3.7 Hz, 4H), 3.86 (d, J = 3.5 Hz, 2H), 2.49- 2.43 (m, 1H), 2.29 (dd, J = 13.1, 6.1 Hz, 1H). LC/MS (ESI) m/z: 553 (M + H)⁺. RT (Method A): 1.00 min. |
| 218^{c} | 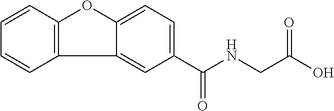 [SMILES: O=C(O)CNC(=O)c1ccc2oc3ccccc3c2c1] | 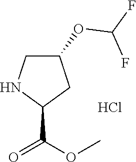 | 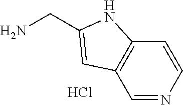 [SMILES: NCc1cc2cnccc2[nH]1 ; Cl] | ¹H NMR (400 MHz, CD₃OD): δ 8.56 (s, 2H), 8,03 (dd, J = 13.1, 8.0 Hz, 2H), 7.83 (d, J = 5.8 Hz, 1H), 7.65 (d, J = 8.3 Hz, 2H), 7.55 (t, J = 7.6 Hz, 1H), 7.41 (d, J = 7.4 Hz, 1H), 7.16 (d, J = 5.8 Hz, 1H), 6.60 (t, J = 41.7 Hz, 2H), 5.06 (s, 1H), 4.70 (d, J = 15.9 Hz, 1H), 4.65-4.54 (m, 2H), 4.30 (d, J = 9.5 Hz, 2H), 4.02 (d, J = 4.5 Hz, 1H), 3.95 (s, 1H), 2.50 (d, J = 11.0 Hz, 1H), 2.35-2.22 (m, 1H). LC/MS (ESI) m/z: 562 (M + H)⁺. RT (Method A): 1.29 min. |
| 235^{c} | 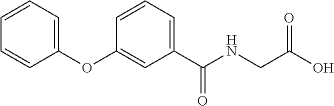 [SMILES: O=C(O)CNC(=O)c1cccc(Oc2ccccc2)c1] | 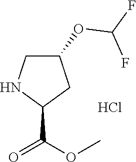 | 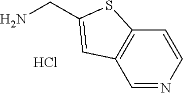 [SMILES: NCc1cc2cnccc2s1 ; Cl] | ¹H NMR (400 MHz, CD₃OD): δ 8.92 (d, J = 26.4 Hz, 1H), 8.28 (d, J = 5.6 Hz, 1H), 7.87 (d, J = 4.8 Hz, 1H), 7.57 (t, J = 9.1 Hz, 1H), 7.48- 7.40 (m, 3H), 7.36 (t, J = 7.8 Hz, 2H), 7.16-7.11 (m, 2H), 7.00 (d, J = 8.4 Hz, 2H), 6.69-6.27 (m, 1H), 5.03-4.97 (m, 1H), 4.76-4.67 (m, 2H), 4.61-4.56 (m, 1H), 4.25-4.14 (m, 2H), 3.97 (dd, J = 11.5, 4.7 Hz, 1H), 3.85 (d, J = 11.8 Hz, 1H), 2.52-2.43 (m, 1H), 2.30- 2.20 (m, 1H). LC/MS (ESI) m/z: 581 (M + H)⁺. RT (Method A): 1.50 min. |
| 246^{c} | 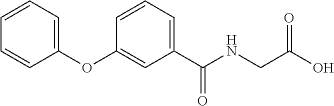 [SMILES: O=C(O)CNC(=O)c1cccc(Oc2ccccc2)c1] | 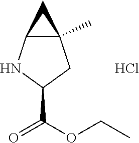 | 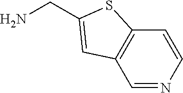 [SMILES: NCc1cc2cnccc2s1] | ¹H NMR (400 MHz, CD₃OD): δ 8.88 (s, 1H), 8.33-8.18 (m, 1H), 7.87 (t, J = 6.9 Hz, 1H), 7.55 (d, J = 7.8 Hz, 1H), 7.49-7.44 (m, 1H), 7.43-7.31 (m, 4H), 7.17-7.08 (m, 2H), 7.06-6.90 (m, 2H), 4.86 (d, J = 3.4 Hz, 1H), 4.64 (s, 2H), 4.32 (q, J = 16.4 Hz, 2H), 3.38 (dd, J = 6.0, 2.4 Hz, 1H), 2.49-2.28 (m, 1H), 2.19 (dd, J = 13.3, 3.3 Hz, 1H), 1.28 (s, 3H), 1.19-1.06 (m, 1H), 0.85-0.67 (m, 1H). LC/MS (ESI) m/z: 541 (M + H)⁺. RT (Method A): 1.45 min. |
| 251^{b,c} | 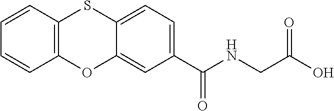 [SMILES: O=C(O)CNC(=O)c1ccc2c(c1)Oc1ccccc1S2] | 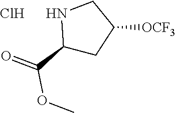 | 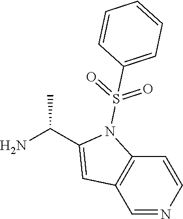 | ¹H NMR (400 MHz, CD₃OD): δ 8.63 (d, J = 0.9 Hz, 1H), 7.88 (d, J = 5.8 Hz, 1H), 7.62-7.55 (m, 2H), 7.26 (d, J = 8.0 Hz, 1H), 7.21- 7.15 (m, 2H), 7.10-7.06 (m, 2H), 7.03 (dd, J = 8.0, 1.2 Hz, 1H), 6.51 (s, 1H), 5.33-5.29 (m, 1H), 5.24 (s, 1H), 4.58 (t, J = 8.1 Hz, 1H), 4.35 (d, J = 16.8 Hz, 1H), 4.15 (d, J = 16.8 Hz, 1H), 4.06-3.98 (m, 2H), 2.60-2.53 (m, 1H), 2.38-2.30 (m, 1H), 1.60 (d, J = 7.0 Hz, 3H). LC/MS (ESI) m/z: 626 (M + H)⁺. RT (Method A): 1.59 min. |
| 252 | 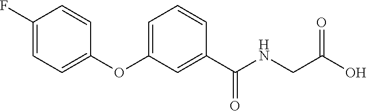 [SMILES: O=C(O)CNC(=O)c1cccc(Oc2ccc(F)cc2)c1] | 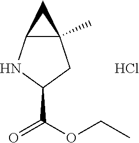 | 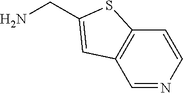 [SMILES: NCc1cc2cnccc2s1] | ¹H NMR (400 MHz, CD₃OD): δ 8.90 (d, J = 3.9 Hz, 1H), 8.28 (d, J = 5.7 Hz, 1H), 7.89 (d, J = 5.7 Hz, 1H), 7.55 (d, J = 7.7 Hz, 1H), 7.44-7.37 (m, 3H), 7.10 (dd, J = 11.8, 5.7 Hz, 3H), 7.04-6.99 (m, 2H), 4.65 (s, 2H), 4.58 (s, 1H), 4.33 (q, J = 22.9, 10.6 Hz, 2H), 3.39 (dd, J = 6.0, 2.4 Hz, 1H), 2.42 (t, J = 12.5 Hz, 1H), 2.19 (dd, J = 13.4, 3.3 Hz, 1H), 1.29 (s, 3H), 1.19-1.14 (m, 1H), 0.82-0.73 (m, 1H). LC/MS (ESI) m/z: 559 (M + H)⁺. RT (Method A): 1.50 min. |
| 254^{b,c} | 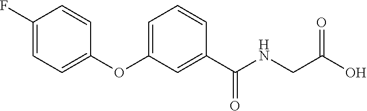 [SMILES: O=C(O)CNC(=O)c1cccc(Oc2ccc(F)cc2)c1] | 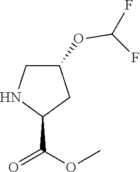 | 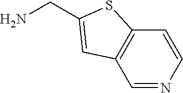 [SMILES: NCc1cc2cnccc2s1] | ¹H NMR (400 MHz, CD₃OD): δ 8.88 (s, 1H), 8.28 (d, J = 5.6 Hz, 1H), 7.87 (d, J = 5.6 Hz, 1H), 7.57 (d, J = 8.0 Hz, 1H), 7.42 (t, J = 7.8 Hz, 3H), 7.14-7.08 (m, 3H), 7.05-6.99 (m, 2H), 6.50 (t, J = 74.5 Hz, 1H), 5.04-4.96 (m, 1H), 4.74-4.67 (m, 2H), 4.59 (t, J = 7.7 Hz, 1H), 4.25-4.14 (m, 2H), 4.00-3.92 (m, 1H), 3.85 (d, J = 12.7 Hz, 1H), 2.54-2.42 (m, 1H), 2.29-2.19 (m, 1H). LC/MS (ESI) m/z: 599 (M + H)⁺. RT (Method A): 1.47 min. |
| 256^{b,c} | 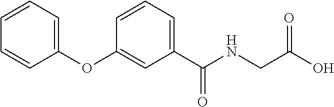 [SMILES: O=C(O)CNC(=O)c1cccc(Oc2ccccc2)c1] | 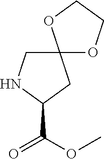 | 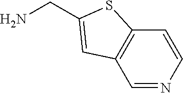 [SMILES: NCc1cc2cnccc2s1] | ¹H NMR (400 MHz, CD₃OD): δ 8.98 (s, 1H), 8.33 (s, 1H), 8.07 (s, 1H), 7.57-7.54 (m, 1H), 7.53-7.48 (m, 1H), 7.46-7.39 (m, 2H), 7.39-7.34 (m, 2H), 7.16-7.10 (m, 2H), 7.03-6.97 (m, 2H), 4.71 (d, J = 7.0 Hz, 2H), 4.62 (dd, J = 9.3, 5.3 Hz, 1H), 4.14 (dd, J = 42.0, 16.5 Hz, 2H), 4.00-3.92 (m, 4H), 3.79 (s, 2H), 2.50-2.33 (m, 1H), 2.32-2.15 (m, 1H). LC/MS (ESI) m/z: 573 (M + H)⁺. RT (Method A): 1.27 min. |
| 301^{b,c} | 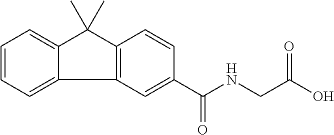 | 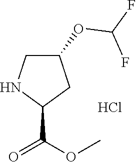 | 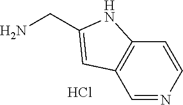 [SMILES: NCc1cc2cnccc2[nH]1 ; Cl] | ¹H-NMR (400 MHz, CD₃OD): δ 8.63 (s, 1H), 8.26 (s, 1H), 7.85 (d, J = 5.9 Hz, 1H), 7.83 (dd, J = 8.0, 1.5 Hz, 1H), 7.79-7.76 (m, 1H), 7.57 (d, J = 8.0 Hz, 1H), 7.53-7.50 (m, 1H), 7.36 (dd, J = 5.5, 3.2 Hz, 2H), 7.22 (d, J = 5.9 Hz, 1H), 6.72-6.36 (m, 2H), 5.08-5.04 (m, 1H), 4.75-4.71 (m, 1H), 4.62-4.54 (m, 2H), 4.29 (d, J = 10.6 Hz, 2H), 4.05-4.01 (m, 1H), 3.95-3.91 (m, 1H), 2.55-2.48 (m, 1H), 2.33-2.26 (m, 1H), 1.51 (s, 6H). LC/MS (ESI) m/z: 588 (M + H)⁺. RT (Method A): 1.53 min. |
| 370^{b,c} | 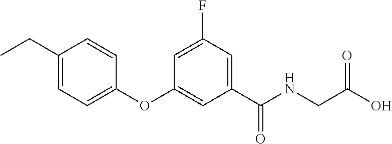 | 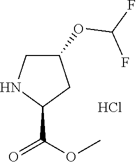 | 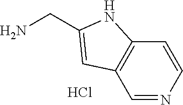 [SMILES: NCc1cc2cnccc2[nH]1 ; Cl] | ¹H NMR (400 MHz, CD₃OD): δ 8.67-8.62 (m, 1H), 8.00 (d, J = 5.8 Hz, 1H), 7.25-7.21 (m, 3H), 7.00-6.95 (m, 2H), 6.88-6.84 (m, 1H), 6.70-6.33 (m, 2H), 5.06-5.00 (m, 1H), 4.71-4.66 (m, 1H), 4.59-4.50 (m, 2H), 4.27-4.13 (m, 2H), 4.00-3.94 (m, 1H), 3.90- 3.85 (m, 1H), 2.67-2.62 (m, 2H), 2.52-2.43 (m, 1H), 2.31-2.22 (m, 1H). LC/MS (ESI) m/z: 610 (M + H)⁺. RT (Method A): 1.75 min. |
| 372^{b,c} | 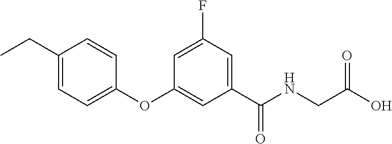 | 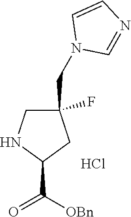 [SMILES: O=C(OCc1ccccc1)[C@@H]1C[C@](F)(Cn2ccnc2)CN1 ; Cl] | 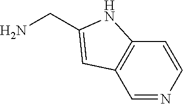 [SMILES: NCc1cc2cnccc2[nH]1] | ¹H NMR (400 MHz, CD₃OD): δ 8.66 (s, 1H), 8.00 (m, 1H), 7.72 (s, 1H), 7.32-7.27 (m, 2H), 7.26-7.19 (m, 4H), 7.02 (s, 1H), 6.98- 6.94 (m, 2H), 6.88-6.83 (m, 1H), 6.53 (s, 1H), 4.71 (d, J = 15.9 Hz, 1H), 4.62-4.58 (m, 4H), 4.24-4.07 (m, 2H), 4.05-3.91 (m, 2H), 2.67-2.61 (m, 2H), 2.48-2.36 (m, 1H), 2.34-2.18 (m, 1H), 1.23 (t, J = 7.6, 1.2 Hz, 3H). LC/MS (ESI) m/z: 642 (M + H)⁺. RT (Method A): 1.19 min. |

^{a}T₃P used in place of PyBOP in Step 3.
^{b}HATU used in place of T₃P in Step 1.
^{c}HATU used in place of PyBOP in Step 3.

### Scheme 10. Synthesis of (S)-N-(oxazolo[4,5-c]pyridin-2-ylmethyl)-7-((phenoxathiine-3-carbonyl)glycyl)-1,4-dioxa-7-azaspiro[4.4]nonane-8-carboxamide (Compound 31)

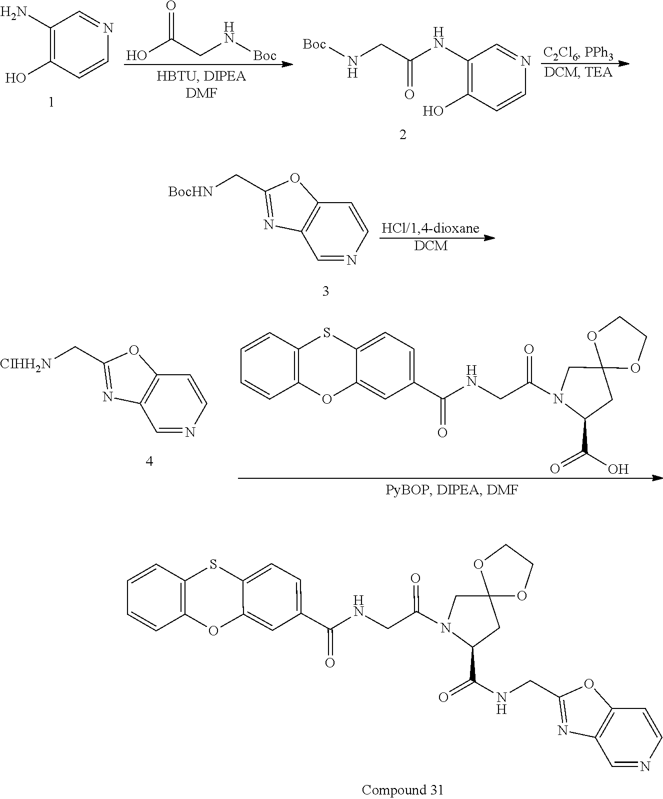

### Step 1: tert-butyl (2-((4-hydroxypyridin-3-yl)amino)-2-oxoethyl)carbamate

To a mixture of 3-aminopyridin-4-ol (2.0 g, 18.18 mmol) and (tert-butoxycarbonyl) glycine (3.8 g, 21.82 mmol) in DMF (20 mL) was added DIPEA (11.7 g, 90.90 mmol) and HBTU (8.6 g, 21.82 mmol) under N₂ atmosphere at 0° C. and the mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-20% MeOH in DCM) to give tert-butyl (2-((4-hydroxypyridin-3-yl)amino)-2-oxoethyl)carbamate (3.75 g, yield 77.3%) as a white solid. LC/MS (ESI) (m/z): 268 (M+H)⁺.

### Step 2: tert-butyl(oxazolo[4,5-c]pyridin-2-ylmethyl)carbamate

To a solution of C₂Cl₆ (1.1 g, 4.68 mmol) in DCM (10 mL) was added PPh₃ (1.5 g, 5.61 mmol) and TEA (2.1 g, 14.96 mmol) and the mixture was stirred at 25° C. for 10 minutes. To the mixture was added tert-butyl (2-((4-hydroxypyridin-3-yl)amino)-2-oxoethyl)carbamate (0.5 g, 1.87 mmol) and the resulting mixture was stirred at 25° C. for 2 hours. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-5% MeOH in DCM) to give tert-butyl(oxazolo[4,5-c]pyridin-2-ylmethyl)carbamate (0.3 g, yield 64.4%) as a brown solid. LC/MS (ESI) m/z: 250 (M+H)⁺.

### Step 3: oxazolo[4,5-c]pyridin-2-ylmethanamine hydrochloride

To a solution of tert-butyl (oxazolo[4,5-c]pyridin-2-ylmethyl)carbamate (0.3 g, 1.20 mmol) in DCM (1 mL) was added HCl/1,4-dioxane (3 mL, 4M) and the mixture was stirred under N₂ atmosphere at room temperature for 2 hours. The reaction mixture was concentrated under reduced pressure to dryness to give oxazolo[4,5-c]pyridin-2-ylmethanamine hydrochloride (150 mg, yield 83.3%) as a brown solid, which was used directly in the next reaction without further purification. LC/MS (ESI) m/z: 150 (M+H)⁺.

### Step 4: (S)-N-(oxazolo[4,5-c]pyridin-2-ylmethyl)-7-((phenoxathiine-3-carbonyl)glycyl)-1,4-dioxa-7-azaspiro[4.4]nonane-8-carboxamide (Compound 31)

To a mixture of (S)-7-((phenoxathiine-3-carbonyl)glycyl)-1,4-dioxa-7-azaspiro[4.4]nonane-8-carboxylic acid (40 mg, 0.09 mmol) and oxazolo[4,5-c]pyridin-2-ylmethanamine hydrochloride (39 mg, 0.27 mmol) in DMF (3 mL) was added DIPEA (70 mg, 0.54 mmol) and PyBOP (55 mg, 0.11 mmol) under N₂ atmosphere at 0° C. and the mixture was stirred at room temperature for 1 hour. The mixture was quenched with saturated aq. NaHCO₃ solution and extracted with CHCl₃/i-PrOH (3/1, v/v) twice. The combined organic layers were washed with brine, dried over anhydrous Na₂SO₄, filtered, and concentrated under reduced pressure to dryness. The residue was purified by prep-TLC (DCM:MeOH=10:1) and further purified by prep-HPLC to give Compound 31 (3 mg, yield 5.9%) as a white solid. ¹H NMR (400 MHz, CD₃OD): δ 8.90 (s, 1H), 8.46 (t, J=4.6 Hz, 1H), 7.68 (dd, J=13.8, 5.7 Hz, 1H), 7.49 (dd, J=8.1, 1.9 Hz, 1H), 7.43 (dd, J=6.4, 1.7 Hz, 1H), 7.19 (dt, J=14.2, 4.9 Hz, 2H), 7.13 (dd, J=7.7, 1.6 Hz, 1H), 7.07 (dd, J=11.2, 3.8 Hz, 1H), 7.04-7.00 (m, 1H), 4.76 (dd, J=15.6, 9.5 Hz, 2H), 4.62 (dd, J=13.5, 7.5 Hz, 1H), 4.23-4.13 (m, 2H), 3.97 (ddd, J=12.3, 6.8, 3.7 Hz, 4H), 3.82-3.72 (m, 2H), 2.46 (dd, J=13.2, 9.0 Hz, 1H), 2.28 (dd, J=13.2, 6.0 Hz, 1H). LC/MS (ESI) (m/z): 588 (M+H)⁺. RT (Method A: 1.73 min.

### Scheme 11. Synthesis of (S)-N-((1H-pyrrolo[3,2-c]pyridin-2-yl)methyl)-7-((4-benzoylbenzoyl)glycyl)-1,4-dioxa-7-azaspiro[4.4]nonane-8-carboxamide (Compound 33)

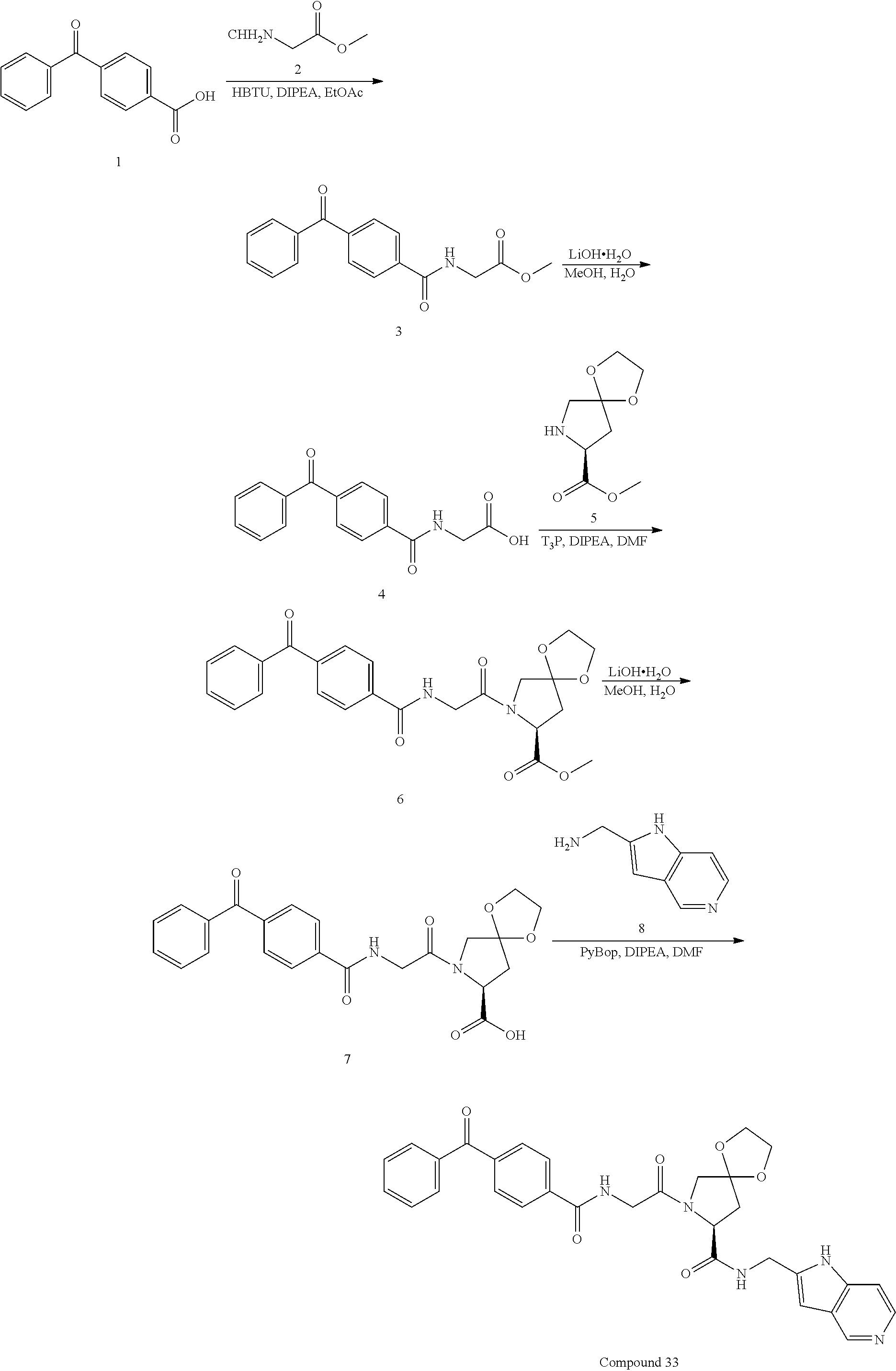

### Step 1: methyl (4-benzoylbenzoyl)glycinate

To a mixture of 4-benzoylbenzoic acid (200 mg, 0.88 mmol) and methyl glycinate hydrochloride (133 mg, 1.06 mmol) in EtOAc (2 mL) was added DIPEA (341 mg, 2.64 mmol) and HBTU (402 mg, 1.06 mmol) under N₂ atmosphere and the mixture was stirred at room temperature for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-60% EtOAc in PE) to give methyl (4-benzoylbenzoyl)glycinate (210 mg, yield 79.9%) as a yellow oil. LC/MS (ESI) m/z: 298 (M+H)⁺.

### Step 2: (4-benzoylbenzoyl)glycine

To a solution of methyl (4-benzoylbenzoyl)glycinate (200 mg, 0.67 mmol) in MeOH (1.5 mL) and water (0.5 mL) was added LiOH·H₂O (63 mg, 1.5 mmol) at 0° C. and the mixture was stirred at room temperature for 3 hours. The reaction mixture was acidified with 1N aq. HCl to pH-3 and extracted with EtOAc twice. The combined organic layers were washed with brine, dried over anhydrous Na₂SO₄, filtered, and concentrated under reduced pressure to dryness to give (4-benzoylbenzoyl)glycine (170 mg, yield 89.6%) as a white solid, which was used in next reaction without purification. LC/MS (ESI) m/z: 284 (M+H)⁺.

### Step 3: methyl (S)-7-((4-benzoylbenzoyl)glycyl)-1,4-dioxa-7-azaspiro[4.4]nonane-8-carboxylate

To a mixture of methyl (S)-1,4-dioxa-7-azaspiro[4.4]nonane-8-carboxylate (150 mg, 0.8 mmol) and (4-benzoylbenzoyl)glycine (150 mg, 0.53 mmol) in DMF (1.5 mL) was added DIPEA (260 mg, 2.0 mmol) and T₃P (509 mg, 0.8 mmol, 50% wt. in EtOAc) under N₂ atmosphere and the mixture was stirred at room temperature for 1 hour. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-80% EtOAc in PE) to give methyl (S)-7-((4-benzoylbenzoyl)glycyl)-1,4-dioxa-7-azaspiro[4.4]nonane-8-carboxylate (140 mg, yield 58.4%) as a yellow oil. LC/MS (ESI) m/z: 453 (M+H)⁺.

### Step 4: (S)-7-((4-benzoylbenzoyl)glycyl)-1,4-dioxa-7-azaspiro[4.4]nonane-8-carboxylic acid

To a solution of methyl (S)-7-((4-benzoylbenzoyl)glycyl)-1,4-dioxa-7-azaspiro[4.4]nonane-8-carboxylate (100 mg, 0.22 mmol) in MeOH (1 mL) and water (0.5 mL) was added LiOH·H₂O (21 mg, 0.5 mmol) at 0° C. and the mixture was stirred at room temperature for 3 hours. The reaction mixture was acidified with 1N aq. HCl to pH-3 and extracted with EtOAc twice. The combined organic layers were washed with brine, dried over anhydrous Na₂SO₄, filtered, and concentrated under reduced pressure to dryness to give (S)-7-((4-benzoylbenzoyl)glycyl)-1,4-dioxa-7-azaspiro[4.4]nonane-8-carboxylic acid (90 mg, yield 93.3%) as a white solid, which was used in next reaction without purification. LC/MS (ESI) n/z: 439 (M+H)⁺.

### Step 5: (S)-N-((1H-pyrrolo[3,2-c]pyridin-2-yl)methyl)-7-((4-benzoylbenzoyl)glycyl)-1,4-dioxa-7-azaspiro[4.4]nonane-8-carboxamide (Compound 33)

To a mixture of (S)-7-((4-benzoylbenzoyl)glycyl)-1,4-dioxa-7-azaspiro[4.4]nonane-8-carboxylic acid (45 mg, 0.10 mmol) and (LH-pyrrolo[3,2-c]pyridin-2-yl)methanamine (17 mg, 0.114 mmol) in DMF (0.5 mL) was added DIPEA (65 mg, 0.5 mmol) and T₃P (95 mg, 0.15 mmol, 50% wt. in EtOAc) under N₂ atmosphere and the mixture was stirred at room temperature for 1 hour. The mixture was diluted with EtOAc, washed with saturated aq. NaIICO₃ solution and brine, dried over anhydrous Na₂SO₄, filtered, and concentrated under reduced pressure to dryness. The residue was purified by prep-TLC (DCM:MeOH=8:1) and further purified by prep-HPLC to give Compound 33 (28 mg, yield 49.3%) as a white solid. ¹H NMR (400 MHz, CD₃OD): δ 8.88 (s, 1H), 8.39 (s, 1H), 8.15 (d, J=6.6 Hz, 1H), 7.94 (d, J=8.3 Hz, 2H), 7.82-7.78 (m, 4H), 7.68 (dd, J=15.7, 7.0 Hz, 2H), 7.56 (t, J=7.7 Hz, 2H), 6.88 (s, 1H), 4.69 (s, 2H), 4.65 (dd, J=8.9, 6.0 Hz, 1H), 4.29 (d, J=16.6 Hz, 1H), 4.19 (d, J=16.6 Hz, 1H), 4.04 (s, 4H), 3.89-3.82 (m, 2H), 2.49 (dd, J=13.1, 9.1 Hz, 1H), 2.29 (dd, J=13.2, 6.0 Hz, 1H). LC/MS (ESI) (m/z): 568 (M+H)⁺. RT (Method A): 1.07 min.

### Scheme 12. Synthesis of (S)-N-((1H-pyrrolo[3,2-b]pyridin-2-yl)methyl)-7-((9,9-difluoro-9H-fluorene-3-carbonyl)glycyl)-1,4-dioxa-7-azaspiro[4.4]nonane-8-carboxamide (Compound 34)

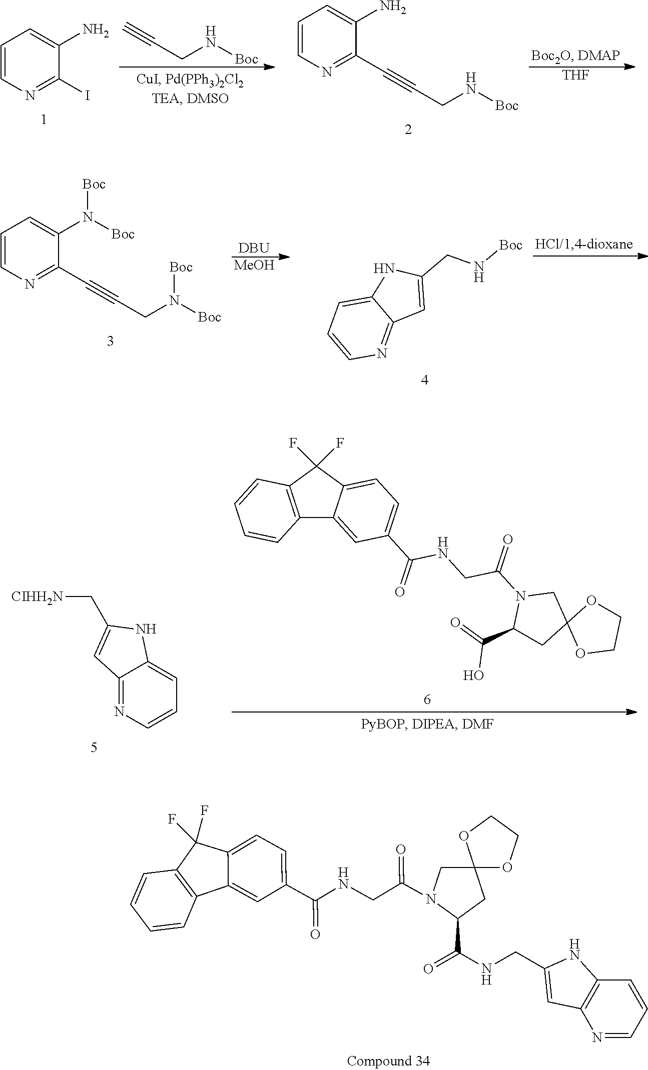

### Step 1: tert-butyl (3-(3-aminopyridin-2-yl)prop-2-yn-1-yl)carbamate

To a mixture of 2-iodopyridin-3-amine (500 mg, 2.3 mmol) and tert-butyl (3-(3-aminopyridin-2-yl)prop-2-yn-1-yl)carbamate (423 mg, 2.7 mmol) in DMSO (5 mL) and TEA (3 mL) was added CuI (53 mg, 0.28 mmol), Pd(dppf)₂Cl₂ (197 mg, 0.28 mmol) under N₂ atmosphere. The mixture was degassed under N₂ atmosphere for three times and stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give tert-butyl (3-(3-aminopyridin-2-yl)prop-2-yn-1-yl)carbamate (400 mg, yield 71.3%) as a brown oil. LC/MS (ESI) m/z: 248 (M+H)⁺.

### Step 2: tert-butyl (3-(3-(bis(tert-butoxycarbonyl)amino)pyridin-2-yl)prop-2-yn-1-yl)(tert-butoxycarbonyl)carbamate

To a solution of tert-butyl (3-(3-aminopyridin-2-yl)prop-2-yn-1-yl)carbamate (400 mg, 1.6 mmol) in THE (4 mL) was added Boc₂O (1.1 g, 4.8 mmol) at 0° C. and the mixture was stirred at 28° C. for 16 hours. The mixture was diluted with EtOAc, washed with water and brine, dried over anhydrous Na₂SO₄, filtered, and concentrated to dryness. The residue was purified by flash chromatography (silica gel, 0-10% EtOAc in PE) to give tert-butyl (3-(3-(bis(tert-butoxycarbonyl)amino)pyridin-2-yl)prop-2-yn-1-yl)(tert-butoxycarbonyl)carbamate (400 mg, yield 71.0%) as a white solid. LC/MS (ESI) m/z: 548 (M+H)⁺.

### Step 3: tert-butyl ((1H-pyrrolo[3,2-b]pyridin-2-yl)methyl)carbamate

To a solution of tert-butyl (3-(3-(bis(tert-butoxycarbonyl)amino)pyridin-2-yl)prop-2-yn-1-yl)(tert-butoxycarbonyl)carbamate (400 mg, 1.6 mmol) in MeOH (4 mL) was added DBU (139 mg, 0.91 mmol) and the reaction mixture was stirred at 60° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give tert-butyl ((1H-pyrrolo[3,2-b]pyridin-2-yl)methyl)carbamate (50 mg, yield 55.5%) as a brown oil. LC/MS (ESI) m/z: 248 (M+H)⁺.

### Step 4: (1H-pyrrolo[3,2-b]pyridin-2-yl)methanamine hydrochloride

A solution of tert-butyl ((1H-pyrrolo[3,2-b]pyridin-2-yl)methyl)carbamate (50 mg, 0.20 mmol) in HCl/1,4-dioxane (2 mL, 4M) was stirred under N₂ atmosphere at room temperature for 2 hours. The reaction mixture was concentrated under reduced pressure to dryness to give ((1H-pyrrolo[3,2-b]pyridin-2-yl)methyl)-13-chromanamine (35 mg, yield 93.7%) as a yellow solid, which was used directly in the next step without further purification. LC/MS (ESI) m/z: 148 (M+H)⁺.

### Step 5: (S)-N-((JH-pyrrolo[3,2-b]pyridin-2-yl)methyl)-7-((9,9-difluoro-9H-fluorene-3-carbonyl)glycyl)-1,4-doxa-7-azaspiro[4.4]nonane-8-carboxamide (Compound 34)

To a mixture of (1H-pyrrolo[3,2-b]pyridin-2-yl)methanamine hydrochloride (29 mg, 0.16 mmol) and (S)-7-((9,9-difluoro-9H-fluorene-3-carbonyl)glycyl)-1,4-dioxa-7-azaspiro [4.4]nonane-8-carboxylic acid (73 mg, 0.16 mmol) in DMF (1.0 ml) was added DIPEA (82 mg, 0.64 mmol) and PyBOP (83 mg, 0.16 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 34 (30 mug, yield 31.9%) as a white solid. ¹H NMR (400 MHz, CD₃OD): δ 8.27 (s, 1H), 8.14 (dd, J=5.2, 1.1 Hz, 1H), 8.05 (s, 1H), 7.82-7.77 (m, 2H), 7.68-7.62 (m, 3H), 7.56 (t, J=7.2 Hz, 1H), 7.44 (t, J=7.5 Hz, 1H), 7.05 (dd, J=8.1, 5.3 Hz, 1H), 6.61 (d, J=23.4 Hz, 1H), 4.70 (s, 2H), 4.64 (dd, J=8.9, 6.0 Hz, 1H), 4.29-4.16 (m, 2H), 4.04 (s, 4H), 3.86 (q, J=10.7 Hz, 2H), 2.49 (dd, J=13.1, 9.0 Hz, 1H), 2.29 (dd, J=13.2, 6.0 Hz, 1H). LC/MS (ESI) m/z: 588 (M+H)⁺. RT (Method A): 1.32 min.

### Scheme 13. Synthesis of (2S,4R)-N-((R)-1-(1H-pyrrolo[3,2-c]pyridin-2-yl)ethyl)-4-(difluoromethoxy)-1-((4-phenoxybenzoyl)glycyl)pyrrolidine-2-carboxamide (Compound 39)

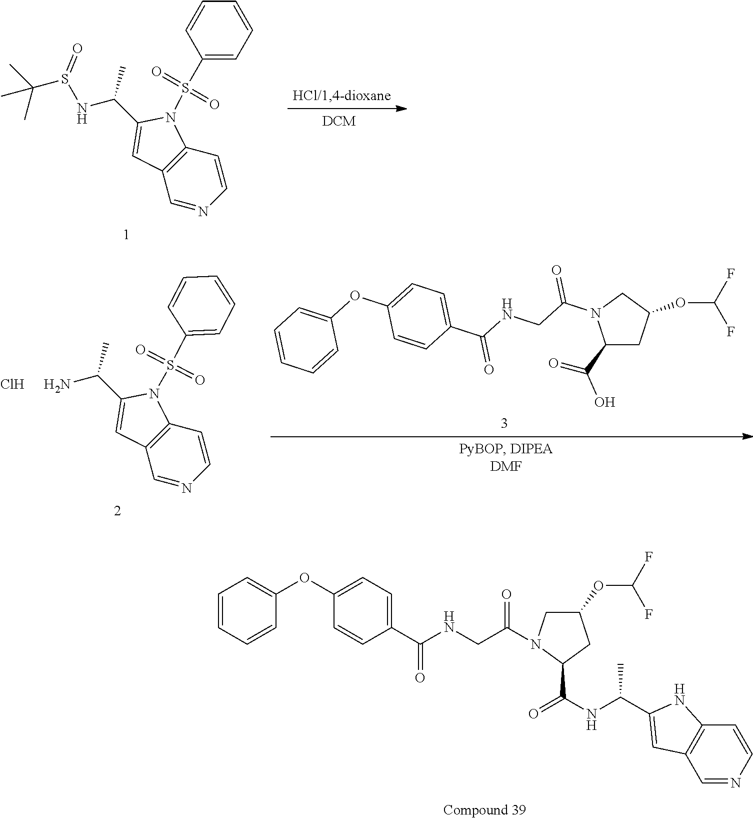 [SMILES: C[C@@H](NS(=O)C(C)(C)C)c1cc2cnccc2n1S(=O)(=O)c1ccccc1 ; Cl ; C[C@@H](N)c1cc2cnccc2n1S(=O)(=O)c1ccccc1 ; O=C(NCC(=O)N1C[C@H](OC(F)F)C[C@H]1C(=O)O)c1ccc(Oc2ccccc2)cc1 ; C[C@@H](NC(=O)[C@@H]1C[C@@H](OC(F)F)CN1C(=O)CNC(=O)c1ccc(Oc2ccccc2)cc1)c1cc2cnccc2[nH]1]

### Step 1: (R)-1-(1-(phenylsulfonyl)-1H-pyrrolo[3,2-c]pyridin-2-yl)ethan-1-amine hydrochloride

A solution of (S)-2-methyl-N-((R)-1-(1-(phenylsulfonyl)-1H-pyrrolo[3,2-c]pyridin-2-yl)ethyl)propane-2-sulfinamide (30 mg, 0.07 mmol) in HCl/1,4-dioxane (2 mL, 4M) was stirred under N₂ atmosphere at room temperature for 2 hours. The reaction mixture was concentrated under reduced pressure to dryness to give (R)-1-(1-(phenylsulfonyl)-1H-pyrrolo[3,2-c]pyridin-2-yl)ethan-1-amine hydrochloride (24 mg, yield 96.0%) as a white solid, which was used directly in the next step without further purification. LC/MS (ESI) m/z: 302 (M+H)⁺.

### Step 2: (2S,4R)-N-((R)-1-(H-pyrrolo[3,2-c]pyridin-2-yl)ethyl)-4-(difluoromethoxy)-1-((4-phenoxybenzoyl)glycyl)pyrrolidine-2-carboxamide (Compound 39)

To a mixture of (R)-1-(1-(phenylsulfonyl)-1H-pyrrolo[3,2-c]pyridin-2-yl)ethan-1-amine hydrochloride (24 mg, 0.07 mmol) and (2S,4R)-4-(difluoromethoxy)-1-((4-phenoxybenzoyl)glycyl)-pyrrolidine-2-carboxylic acid (30 mg, 0.07 mmol) in DMF (2 mL) was added DIPEA (45 mg, 0.35 mmol) and PyBOP (54 mg, 0.10 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=10:1) and further purified by prep-HPLC to give Compound 39 (5.2 mg, yield 13.0%) as a white solid. ¹H NMR (400 MHz, CD₃OD): δ 8.84 (d, J=5.2 Hz, 1H), 8.46 (s, 1H), 8.00 (s, 1H), 7.91 (d, J=8.3 Hz, 2H), 7.42 (d, J=8.1 Hz, 3H), 7.22 (t, J=7.5 Hz, 1H), 7.09 (d, J=8.3 Hz, 2H), 7.04 (d, J=8.4 Hz, 2H), 6.76 (s, 1H), 6.53 (t, J=74.5 Hz, 1H), 5.35-5.30 (m, 1H), 5.07 (s, 1H), 4.58 (t, J=7.8 Hz, 1H), 4.33 (d, J=16.8 Hz, 1H), 4.18 (d, J=16.6 Hz, 1H), 3.98 (dd, J=11.5, 3.9 Hz, 1H), 3.91 (d, J=12.0 Hz, 1H), 2.48 (s, 1H), 2.29 (d, J=12.9 Hz, 1H), 1.64 (t, J=9.8 Hz, 3H). LC/MS (ESI) m/z: 578 (M+H)⁺. RT (Method A): 1.39 min.

### Scheme 14. Synthesis of (S)-7-((phenoxathiine-3-carbonyl)glycyl)-N-(thiazolo[4,5-c]pyridin-2-ylmethyl)-1,4-dioxa-7-azaspiro[4.4]nonane-8-carboxamide (Compound 41)

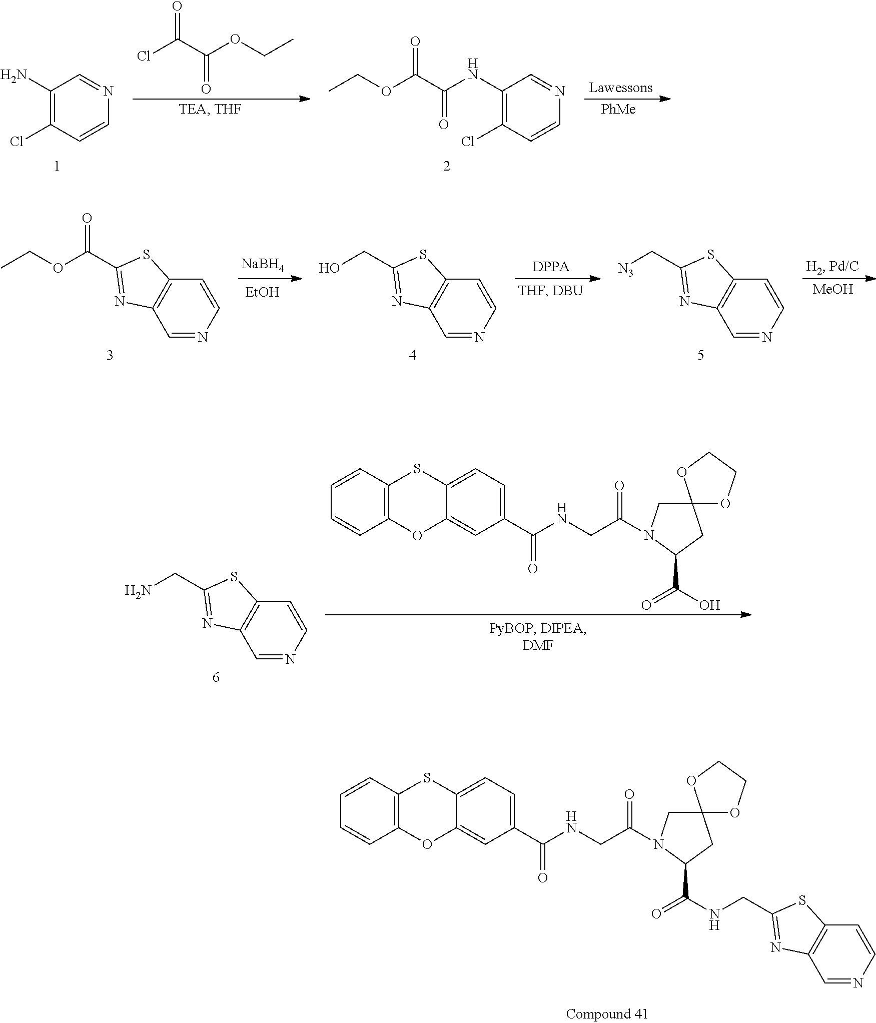

### Step 1: ethyl 2-((4-chloropyridin-3-yl)amino)-2-oxoacetate

To a mixture of 4-chloropyridin-3-amine (1.0 g, 7.81 mmol) and ethyl 2-chloro-2-oxoacetate (1.17 g, 8.6 mmol) in THF (20 mL) was added TEA (0.94 g, 9.31 mmol) at 0° C. under N₂ atmosphere and the reaction mixture was stirred at room temperature for 1 hour. The mixture was diluted with EtOAc, washed with saturated aq. NaHCO₃ solution and brine, dried over anhydrous Na₂SO₄, filtered, and concentrated under reduced pressure to dryness to give ethyl 2-((4-chloropyridin-3-yl)amino)-2-oxoacetate (1.6 g, yield 89.8) as a brown oil, which was used directly in the next step without further purification. LC/MS (ESI) m/z: 229 (M+H)⁺.

### Step 2: ethyl thiazolo[4,5-c]pyridine-2-carboxylate

To a solution of ethyl 2-((4-chloropyridin-3-yl)amino)-2-oxoacetate (1.6 g, 7.02 mmol) in toluene (23 mL) was added Lawessons (1.7 g, 4.21 mmol) under N₂ atmosphere and the reaction mixture was stirred at 115° C. for 2 hours. The mixture was diluted with DCM, washed with saturated aq. NaHCO₃ solution and brine, dried over anhydrous Na₂SO₄, filtered, and concentrated under reduced pressure to dryness. The residue was purified by flash chromatography (silica gel, 0-31% EtOAc in PE) to give ethyl thiazolo[4,5-c]pyridine-2-carboxylate (900 mg, yield 61.6%) as a white solid. LC/MS (ESI) m/z: 209 (M+H)⁺.

### Step 3: thiazolo[4,5-c]pyridin-2-ylmethanol

To a solution of ethyl thiazolo[4,5-c]pyridine-2-carboxylate (900 mg, 4.33 nmol) in EtOH (18 mL) was added NaBH₄ (163.7 mg, 4.33 mmol) at 0° C. and the mixture was stirred at room temperature for 1 hour. The mixture was quenched with ice-water and extracted with EtOAc twice. The combined organic layers were washed with brine, dried over anhydrous Na₂SO₄, filtered, and concentrated under reduced pressure to dryness. The residue was purified by flash chromatography (silica gel, 0-83% EtOAc in PE) to give thiazolo[4,5-c]pyridin-2-ylmethanol (300 mg, yield 41.8%) as a white solid. LC/MS (ESI) m/z: 167 (M+H)⁺.

### Step 4: 2-(azidomethyl)thiazolo[4,5-c]pyridine

To a solution of thiazolo[4,5-c]pyridin-2-ylmethanol (300 mg, 1.81 mmol) in THF (15 mL) was added DPPA (994.7 mg, 1.62 mmol) and DBU (220.1 mg, 1.45 mmol) at 0° C. under N₂ atmosphere and the mixture was stirred at room temperature for 6 hour. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-33% EtOAc in PE) to give 2-(azidomethyl)thiazolo[4,5-c]pyridine (220 mg, yield 63.8%) as a brown solid. LC/MS (ESI) m/z: 192 (M+H)⁺.

### Step 5: thiazolo[4,5-c]pyridin-2-ylmethanamine

To a solution of 2-(azidomethyl)thiazolo[4,5-c]pyridine (220 mg, 1.15 mmol) in MeOH (3 mL) was added Pd/C (22 mg, 10% wt.), the mixture was degassed under N₂ atmosphere for three times and stirred under a H₂ balloon at 25° C. overnight. The mixture was filtered, and the filtrate was concentrated to dryness to give thiazolo[4,5-c]pyridin-2-ylmethanamine (135 mg, yield 71.1%) as a brown solid, which was used directly in the next step without further purification. LC/MS (ESI) m/z: 166 (M+H)⁺.

### Step 6: (S)-7-((phenoxathiine-3-carbonyl)glycyl)-N-(thiazolo[4,5-c]pyridin-2-ylmethyl)-1,4-dioxa-7-azaspiro[4.4]nonane-8-carboxamide (Compound 41)

To a mixture of (S)-7-((phenoxathiine-3-carbonyl)glycyl)-1,4-dioxa-7-azaspiro[4.4]nonane-8-carboxylic acid (40 mg, 0.09 mmol) and thiazolo[4,5-c]pyridin-2-ylmethanamine (29.0 mg, 0.18 mmol) in DMF (2 mL) was added DIPEA (67.9 mg, 0.54 mmol) and PyBOP (54.7 mg, 0.11 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=12:1) and further purified by prep-HPLC to give Compound 41 (20 mg, yield 37.8%) as a white solid. ¹H NMR (400 MHz, CD₃OD): δ 9.10 (s, 1H), 8.36 (d, J=5.5 Hz, 1H), 8.08-7.95 (m, 1H), 7.48 (dd, J=8.1, 1.8 Hz, 1H), 7.42 (d, J=1.8 Hz, 1H), 7.20 (dd, J=10.5, 4.8 Hz, 1H), 7.15 (dd, J=7.8, 1.7 Hz, 2H), 7.10-7.06 (m, 1H), 7.02 (dd, J=8.0, 1.1 Hz, 1H), 4.93-4.88 (m, 1H), 4.78 (s, 1H), 4.67 (dd, J=9.2, 5.3 Hz, 1H), 4.19 (d, J=16.6 Hz, 1H), 4.11 (d, J=16.5 Hz, 1H), 4.01 (s, 3H), 3.81 (d, J=12.3 Hz, 2H), 3.16 (td, J=6.7, 3.7 Hz, 1H), 2.49 (dd, J=13.2, 9.3 Hz, 1H), 2.28 (dd, J=13.2, 5.3 Hz, 1H). LC/MS (ESI) m/z: 604 (M+H)⁺. RT (Method A): 1.76 min.

### Scheme 15. Synthesis of (S)-N-((7-fluoro-1H-pyrrolo[3,2-c]pyridin-2-yl)methyl)-7-((phenoxathiine-3-carbonyl)glycyl)-1,4-dioxa-7-azaspiro[4.4]nonane-8-carboxamide (Compound 45)

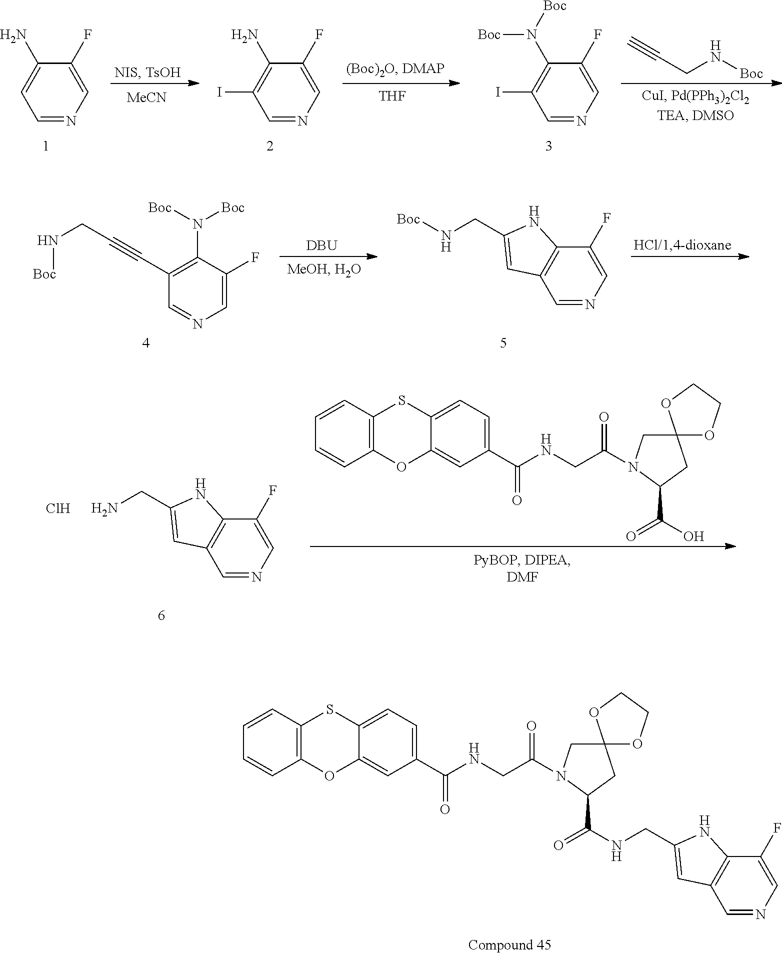

### Step 1: 3-fluoro-5-iodopyridin-4-amine (2)

To a solution of 3-fluoropyridin-4-amine (1.0 g, 8.93 mmol) in MeCN (10 mL) was added NIS (2.4 g, 10.7 mmol) and TsOH (85 mg, 0.45 mmol) under N₂ atmosphere and the reaction mixture was stirred at 70° C. overnight. The mixture was quenched with saturated aq. Na₂S₂O₃ solution and extracted with EtOAc twice. The combined organic layers were washed with brine, dried over anhydrous Na₂SO₄, filtered, and concentrated under reduced pressure to dryness to give 3-fluoro-5-iodopyridin-4-amine (1.5 g, yield 71.4%) as a yellow oil. LC/MS (ESI) m/z: 239 (M+H)⁺.

### Step 2: tert-butyl (tert-butoxycarbonyl)(3-fluoro-5-iodopyridin-4-yl)carbamate (2)

To a solution of 3-fluoro-5-iodopyridin-4-amine (1.5 g, 6.30 mmol) in THE (20 mL) was added (Boc)₂O (6.9 g, 31.5 mmol) and DMAP (76.9 mg, 0.63 mmol) at 0° C. and the mixture was stirred at room temperature for 16 hours. The mixture was diluted with water and extracted with EtOAc twice. The combined organic layers were washed with brine, dried over anhydrous Na₂SO₄, filtered, and concentrated to dryness. The residue was purified by flash chromatography (silica gel, 0-10% EtOAc in PE) to give tert-butyl (tert-butoxycarbonyl)(3-fluoro-5-iodopyridin-4-yl)carbamate (180 mg, yield 6.5%) as a yellow oil. LC/MS (ESI) m/z: 439 (M+H)⁺.

### Step 3: tert-butyl (tert-butoxycarbonyl)(3-(3-((tert-butoxycarbonyl)amino)prop-1-yn-1-yl)-5-fluoropyridin-4-yl)carbamate (4)

To a mixture of tert-butyl (tert-butoxycarbonyl)(3-fluoro-5-iodopyridin-4-yl)carbamate (180 mg, 0.41 mmol) and tert-butyl prop-2-yn-1-ylcarbamate (76.4 mg, 0.49 mmol) in TEA (2 mL) and DMSO (2 mL) was added Cut (4 mg, 0.02 mmol) and Pd(PPh₃)₂Cl₂ (14.4 mg, 0.24 mmol) under N₂ atmosphere, the mixture was degassed under N₂ atmosphere for three times and stirred at 50° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give tert-butyl (tert-butoxycarbonyl)(3-(3-((tert-butoxycarbonyl)amino)prop-1-yn-1-yl)-5-fluoropyridin-4-yl)carbamate (80 mg, yield 41.8%) as a brown oil. LC/MS (ESI) m/z: 466 (M+H)⁺.

### Step 4: tert-butyl ((7-fluoro-1H-pyrrolo[3,2-c]pyridin-2-yl)methyl)carbamate (5)

To a solution of tert-butyl (tert-butoxycarbonyl)(3-(3-((tert-butoxycarbonyl)amino) prop-1-yn-1-yl)-5-fluoropyridin-4-yl)carbamate (80 mg, 0.17 mmol) in MeOH (1 mL)/H₂O (1 mL) was added DBU (53 mg, 0.35 mmol) and the reaction mixture was stirred at 90° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-15% EtOAc in PE) to give tert-butyl ((7-fluoro-1H-pyrrolo[3,2-c]pyridin-2-yl)methyl)carbamate (15 mg, yield 32.9%) as a yellow oil. LC/MS (ESI) in/z: 266 (M+H)⁺.

### Step 5: (7-fluoro-1H-pyrrolo[3,2-c]pyridin-2-yl)methanamine hydrochloride (6)

A solution of tert-butyl ((7-fluoro-1H-pyrrolo[3,2-c]pyridin-2-yl)methyl)carbamate (15 mg, 0.06 mmol) in HCl/1,4-dioxane (2 mL, 4M) was stirred under N₂ atmosphere at room temperature for 2 hours. The reaction mixture was concentrated to dryness under reduced pressure to give (7-fluoro-1H-pyrrolo[3,2-c]pyridin-2-yl)methanamine hydrochloride (10 mg, yield 87.7%) as a yellow solid, which was used directly in the next step without further purification. LC/MS (ESI) m/z: 166 (M+H)⁺.

### Step 6: (S)-N-((7-fluoro-1H-pyrrolo[3,2-c]pyridin-2-yl)methyl)-7-((phenoxathiine-3-carbonyl)glycyl)-1,4-dioxa-7-azaspiro[4.4]nonane-8-carboxamide (Compound 45)

To a mixture of (S)-7-((phenoxathiine-3-carbonyl)glycyl)-1,4-dioxa-7-azaspiro[4.4]nonane-8-carboxylic acid (30 mg, 0.07 mmol) and (7-fluoro-1H-pyrrolo[3,2-c]pyridin-2-yl)methanamine hydrochloride (10 mg, 0.05 mmol) in DMF (2 mL) was added DIPEA (42 mg, 0.33 mmol) and PyBOP (51 mg, 0.10 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was firstly purified by prep-TLC (DCM:MeOH=10:1) and further purified by prep-HPLC to give Compound 45 (1.2 mg, yield 4.0%) as a white solid. ¹H NMR (400 MHz, CD₃OD): δ 7.74 (d, J=5.6 Hz, 1H), 7.55 (dd, J=8.1, 1.9 Hz, 1H), 7.50 (d, J=1.8 Hz, 1H), 7.22-7.13 (m, 4H), 7.08 (d, J=7.4 Hz, 1H), 7.04 (s, 1H), 6.69 (dd, J=7.2, 5.6 Hz, 1H), 4.58-4.53 (m, 1H), 4.33-4.27 (m, 2H), 4.17 (d, J=14.0 Hz, 2H), 3.99 (d, J=1.8 Hz, 4H), 3.76 (t, J=7.2 Hz, 2H), 2.42 (dd, J=13.2, 8.7 Hz, 1H), 2.22 (dd, J=13.0, 6.2 Hz, 1H). LC/MS (ESI) m/z: 604 (M+H)⁺. RT (Method A): 1.42 min.

### Scheme 16. Synthesis of (S)-N-((1-methyl-1H-pyrrolo[3,2-c]pyridin-2-yl)methyl)-7-((phenoxathiine-3-carbonyl)glycyl)-1,4-dioxa-7-azaspiro[4.4]nonane-8-carboxamide (Compound 46)

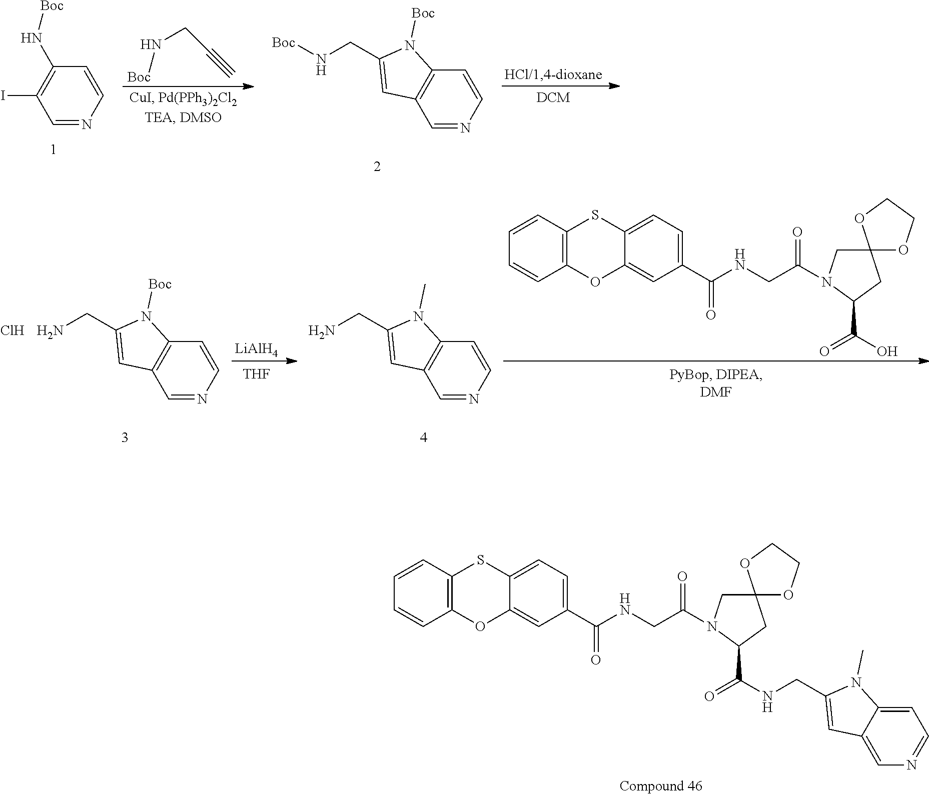

### Step 1: tert-butyl 2-(((tert-butoxycarbonyl)amino)methyl)-H-pyrrolo[3,2-c]pyridine-1-carboxylate (2)

To a mixture of tert-butyl (3-iodopyridin-4-yl)carbamate (2.0 g, 6.25 mmol) and tert-butyl prop-2-yn-1-ylcarbamate (1.2 g, 7.50 mmol) in TEA (10 mL) and DMSO (5 mL) was added CuI (59 mg, 0.31 mmol) and Pd(PPh₃)₂Cl₂ (219 mg, 0.31 mmol) under N₂ atmosphere, the mixture was degassed under N₂ atmosphere for three times and stirred at 50° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give tert-butyl 2-(((tert-butoxycarbonyl)amino)methyl)-1H-pyrrolo[3,2-c]pyridine-1-carboxylate (2.0 g, yield 92.2%) as a brown oil. LC/MS (ESI) m/z: 348 (M+H)⁺.

### Step 2: tert-butyl 2-(aminomethyl)-1H-pyrrolo[3,2-c]pyridine-1-carboxylate hydrochloride (3)

To a solution of tert-butyl 2-(((tert-butoxycarbonyl)amino)methyl)-1H-pyrrolo[3,2-c]pyridine-1-carboxylate (2.0 g, 5.76 mmol) in DCM (10 mL) was added HCl/1,4-dioxane (10 mL, 4M) under N₂ atmosphere and the reaction mixture was stirred at room temperature for 2 hours. The reaction mixture was concentrated under reduced pressure to dryness to give tert-butyl 2-(aminomethyl)-1H-pyrrolo[3,2-c]pyridine-1-carboxylate hydrochloride (1.03 g, yield 63.1%) as a yellow solid, which was used directly in the next step without further purification. LC/MS (ESI) m/z: 248 (M+H)⁺.

### Step 3: (1-methyl-1H-pyrrolo[3,2-c]pyridin-2-yl)methanamine (4)

To a solution of tert-butyl 2-(aminomethyl)-1H-pyrrolo[3,2-c]pyridine-1-carboxylate hydrochloride (200 mg, 0.70 mmol) in THF (20 mL) was added LiAlH₄ (53 mg, 1.41 mmol) under N₂ atmosphere and the reaction mixture was stirred at 90° C. overnight.

The reaction mixture was quenched with Na₂SO₄.10H₂O at 0° C. and stirred vigorously for 10 mins. The mixture was filtered, and the filtrate was concentrated to dryness. The residue was purified by flash chromatography (silica gel, 0-20% McOH in DCM) to give (1-methyl-1H-pyrrolo[3,2-c]pyridin-2-yl)methanamine (40 mg, yield 35.2%) as a white solid. LC/MS (ESI) m/z: 162 (M+H)⁺.

### Step 4: (S)-N-((I-methyl-1H-pyrrolo[3,2-c]pyridin-2-yl)methyl)-7-((phenoxathiine-3-carbonyl)glycyl)-1,4-dioxa-7-azaspiro[4.4]nonane-8-carboxamide (Compound 46)

To a mixture of (S)-7-((phenoxathiine-3-carbonyl)glycyl)-1,4-dioxa-7-azaspiro[4.4]nonane-8-carboxylic acid (50 mg, 0.11 mmol) and (1-methyl-1H-pyrrolo[3,2-c]pyridin-2-yl)methanamine (26 mg, 0.16 mmol) in DMF (5 mL) was added DIPEA (85 mg, 0.66 mmol) and PyBOP (86 mg, 0.16 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=5:1) and further purified by prep-HPLC to give Compound 46 (10 mg, yield 15.2%) as a white solid. ¹H NMR (400 MHz, CD₃OD): δ 8.86 (dd, J=15.6, 8.7 Hz, 1H), 8.47 (s, 1H), 8.20-8.02 (m, 1H), 7.67-7.44 (m, 3H), 7.30-7.14 (m, 3H), 7.11-6.98 (m, 2H), 6.84-6.74 (m, 1H), 5.29-4.97 (m, 2H), 4.76-4.30 (m, 2H), 4.16-3.95 (m, 5H), 3.83 (dt, J=17.0, 11.5 Hz, 2H), 3.20-2.91 (m, 3H), 2.78-2.44 (m, 1H), 2.21 (ddd, J=21.4, 13.4, 6.4 Hz, 1H). LC/MS (ESI) m/z: 600 (M+H)⁺. RT (Method A): 1.35 min.

### Scheme 17. Synthesis of (S)-N-((1H-imidazo[4,5-c]pyridin-2-yl)methyl)-7-((phenoxathiine-3-carbonyl)glycyl)-1,4-dioxa-7-azaspiro[4.4]nonane-8-carboxamide (Compound 49)

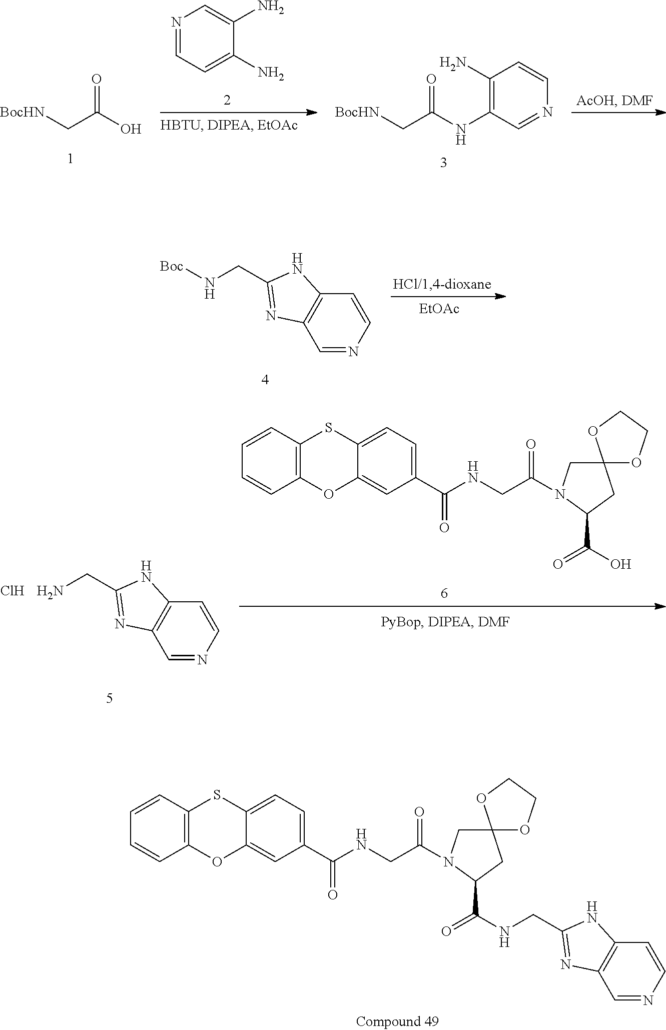

### Step 1: tert-butyl (2-((4-aminopyridin-3-yl)amino)-2-oxoethyl)carbamate

To a mixture of (tert-butoxycarbonyl)glycine (701 mg, 4.0 mmol) and pyridine-3,4-diamine (524 mg, 4.8 mmol) in EtOAc (10 mL) was added DIPEA (1.55 g, 12.0 mmol) and HBTU (1.82 g, 4.8 mmol) under N₂ atmosphere and the mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-80% EtOAc in PE) to give tert-butyl (2-((4-aminopyridin-3-yl)amino)-2-oxoethyl)carbamate (980 mg, yield 92%) as a yellow oil. LC/MS (ESI) m/z: 267 (M+H)⁺.

### Step 2: tert-butyl ((1H-imidazo[4,5-c]pyridin-2-yl)methyl)carbamate

To a mixture of tert-butyl (2-((4-aminopyridin-3-yl)amino)-2-oxoethyl)carbamate (200 mg, 0.75 mmol) in DMF (2 mL) was added AcOH (2 mL) under N₂ atmosphere, the mixture was degassed under N₂ atmosphere for three times and stirred at 120° C. for 3 hours. The reaction mixture was concentrated under reduced pressure to dryness to give tert-butyl ((1H-imidazo[4,5-c]pyridin-2-yl)methyl)carbamate (187 mg, yield 100%) as a brown solid. LC/MS (ESI) m/z: 249 (M+H)⁺.

### Step 3: (1H-imidazo[4,5-c]pyridin-2-yl)methanamine hydrochloride

A solution of tert-butyl ((1H-imidazo[4,5-c]pyridin-2-yl)methyl)carbamate (187 mg, 0.75 mmol) in EtOAc (1 mL) and HCl/1,4-dioxane (1 mL, 4M) was stirred under N₂ atmosphere at room temperature for 2 hours. The reaction mixture was filtered and the solid was washed with EtOAc. The solid was concentrated under reduced pressure to dryness to give (1H-imidazo[4,5-c]pyridin-2-yl)methanamine hydrochloride (95 mg, yield 68.6%) as a yellow solid, which was used directly in the next reaction without further purification. LC/MS (ESI) m/z: 149 (M+H)⁺.

### Step 4: (S)-N-((1H-imidazo[4,5-c]pyridin-2-yl)methyl)-7-((phenoxathiine-3-carbonyl) glycyl)-1,4-dioxa-7-azaspiro[4.4]nonane-8-carboxamide (Compound 49)

To a mixture of (S)-7-((phenoxathiine-3-carbonyl)glycyl)-1,4-dioxa-7-azaspiro[4.4]nonane-8-carboxylic acid (20 mg, 0.044 mmol) and (1H-imidazo[4,5-c]pyridin-2-yl)methanamine hydrochloride (20 mg, 0.11 mmol) in DMF (0.5 mL) was added DIPEA (29 mg, 0.22 mmol) and PyBOP (23 mg, 0.044 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=8:1) and further purified by prep-HPLC to give Compound 49 (12 mug, yield 46.5%) as a white solid. ¹H NMR (400 MHz, CD₃OD): δ 8.71 (s, 1H), 8.11 (s, 1H), 7.47 (d, J=7.9 Hz, 1H), 7.38 (s, 1H), 7.23-7.15 (m, 4H), 7.12-6.99 (m, 3H), 4.75 (d, J=3.8 Hz, 2H), 4.62 (d, J=7.7 Hz, 1H), 4.18 (d, J=9.0 Hz, 2H), 4.03 (d. J=4.8 Hz, 4H), 3.84 (d, J=10.0 Hz, 2H), 2.51-2.44 (m, 1H), 2.30 (dd, J=13.2, 6.7 Hz, 1H). LC/MS (ESI) m/z: 587 (M+H)⁺. RT (Method A): 1.33 min.

### Scheme 18. Synthesis of (S)-N-((1-aminoisoquinolin-7-yl)methyl)-7-((phenoxathiine-3-carbonyl)glycyl)-1,4-dioxa-7-azaspiro[4.4]nonane-8-carboxamide (Compound 51)

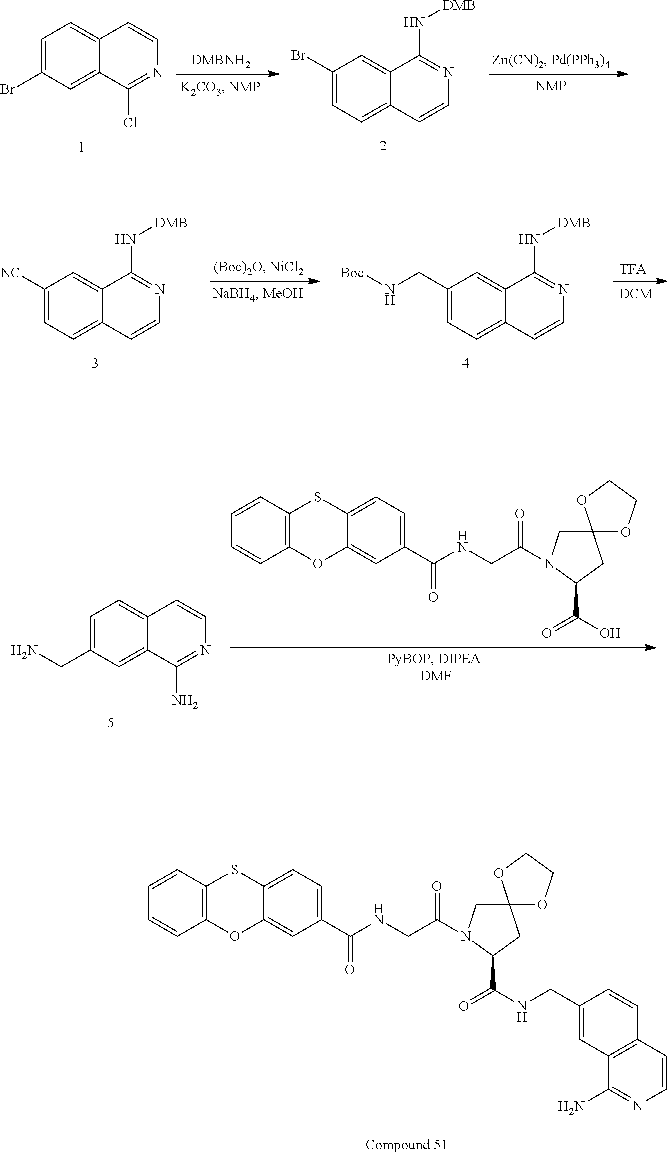

### Step 1: 7-Bromo-V-(2,4-dimethoxybenzyl)isoquinolin-1-amine

To a mixture of 7-bromo-1-chloroisoquinoline (310 mg, 1.29 mmol) and (2,4-dimethoxyphenyl)methanamine (385 mg, 2.31 mmol) in NMP (6 mL) was added K₂CO₃ (850 mg, 6.16 mmol), the reaction mixture was degassed under N₂ atmosphere for three times and stirred at 100° C. for 2 days. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-17% EtOAc in PE) to give 7-bromo-N-(2,4-dimethoxybenzyl)isoquinolin-1-amine (300 mg, yield 62.8%) as a yellow oil. LC/MS (ESI) m/z: 373 (M+H)⁺.

### Step 2: 1-((2,4-Dimethoxybenzyl)amino)isoquinoline-7-carbonitrile

To a mixture of 7-bromo-N-(2,4-dimethoxybenzyl)isoquinolin-1-amine (300 mg, 0.81 mmol) and Zn(CN)₂ (189 Ing, 1.61 mmol) in NMP (6 mL) was added Pd(PPh₃)₄ (468 mg. 0.40 mmol) under N₂ atmosphere. The mixture was degassed under N₂ atmosphere for three times and stirred under N₂ atmosphere at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-25% EtOAc in PE) to give 1-((2,4-dimethoxybenzyl)amino)isoquinoline-7-carbonitrile (253 mg, yield 98.5%) as a yellow solid. LC/MS (ESI) m/z: 320 (M+H)⁺.

### Step 3: Tert-butyl ((1-((2,4-dimethoxybenzyl)amino)isoquinolin-7-yl)methyl) carbamate

To a solution of 1-((2,4-dimethoxybenzyl)amino)isoquinoline-7-carbonitrile (250 mg, 0.78 mmol) in MeOH (5 mL) was added NiCl₂ (19 mg, 0.08 mmol), (Boc)₂O (510 mg, 2.34 mmol) and NaBH₄ (207 mg, 5.47 mmol) at 0° C. and the mixture was stirred at 25° C. for 16 hours. The mixture was quenched with aq. NaHCO₃ solution and extracted with DCM twice. The combined organic layers were washed with brine, dried over anhydrous Na₂SO₄, filtered, and concentrated to dryness. The residue was purified by flash chromatography (silica gel, 0-10% McOH in DCM) to give tert-butyl ((1-((2,4-dimethoxybenzyl)amino)isoquinolin-7-yl)methyl)carbamate (30 mg, yield 9.1%) as a light oil. LC/MS (ESI) m/z: 424(M+H)⁺.

### Step 4: 7-(Aminomethyl)isoquinolin-1-amine

To a solution of tert-butyl ((1-((2,4-dimethoxybenzyl)amino)isoquinolin-7-yl)methyl)carbamate (30 mg, 0.071 mmol) in DCM (2 mL) was added TFA (1 mL) and the mixture was stirred under N₂ atmosphere at room temperature for 4 hours. The reaction mixture was concentrated under reduced pressure to dryness to give 7-(aminomethyl)isoquinolin-1-amine (12 mg, yield 97.8%) as a white solid, which was used directly in the next step without further purification. LC/MS (ESI) m/z: 174 (M+H)⁺.

### Step 5: (S)-N-((-aminoisoquinolin-7-yl)methyl)-7-((phenoxathiine-3-carbonyl)glycyl)-1,4-dioxa-7-azaspiro[4.4]nonane-8-carboxamide (Compound 51)

To a mixture of 7-(aminomethyl)isoquinolin-1-amine (12 mg, 0.069 mmol) and (S)-7-((phenoxathiine-3-carbonyl)glycyl)-1,4-dioxa-7-azaspiro[4.4]nonane-8-carboxylic acid (30 mg, 0.066 mmol) in DMF (3 mL) was added DIPEA (45 mg, 0.35 mmol) and PyBOP (54 mg, 0.10 mmol) under N₂ atmosphere and the reaction mixture was stirred at room temperature for 1 hour. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=10:1) and further purified by prep-HPLC to give Compound 51 (2.9 mg, yield 2.1%) as a white solid. ¹H NMR (400 MHz, CD₃OD): δ 8.09 (s, 1H), 7.78-7.70 (m, 1H), 7.63 (d, J=8.4 Hz, 1H), 7.30-7.22 (m, 3H), 7.17-7.10 (m, 2H), 7.01-6.95 (m, 3H), 6.84 (d, J=6.5 Hz, 1H), 4.93 (d, J=16.3 Hz, 1H), 4.82-4.79 (m, 1H), 4.71 (dd, J=9.3, 5.5 Hz, 1H), 4.45 (d, J=16.4 Hz, 1H), 4.30 (d, J=15.8 Hz, 1H), 4.04 (dd, J=7.2, 4.4 Hz, 4H), 3.96 (d. J=15.9 Hz, 1H), 3.87 (d, J=10.7 Hz, 1H), 2.56 (dd, J=13.0, 9.4 Hz, 1H), 2.29 (dd, J=13.1, 5.3 Hz, 1H).

LC/MS (ESI) m/z: 612 (M+H)⁺. RT (Method A): 1.44 min.

### Scheme 19. Synthesis of (S)-7-((phenoxathiine-3-carbonyl)glycyl)-N-(thieno[3,2-c]pyridin-2-ylmethyl)-1,4-dioxa-7-azaspiro[4.4]nonane-8-carboxamide (Compound 54)

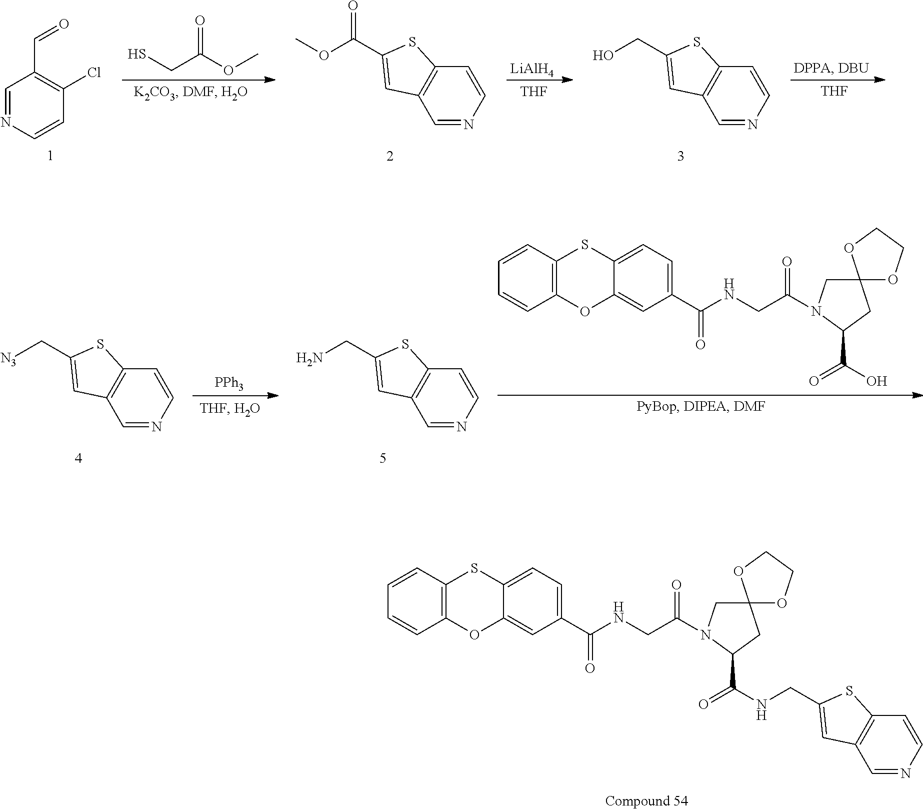

### Step 1: Methyl thieno[3,2-c]pyridine-2-carboxylate

To a solution of methyl 2-mercaptoacetate (1.80 g, 16.96 mmol) in DMF (16.5 mL) and water (5.5 mL) was added K₂CO₃ (2.34 g, 16.96 mmol) at room temperature under N₂ atmosphere and the reaction mixture was stirred at room temperature for 1 hour. 4-Chloronicotinaldehyde (1.80 g, 14.13 mmol) was added to the mixture and the reaction mixture was stirred at 60° C. overnight. The mixture was quenched with saturated aq. NH₄Cl solution and extracted with EtOAc twice. The mixture was concentrated under reduced pressure to dryness. The residue was purified by flash chromatography (silica gel, 0-100% EtOAc in PE) to give methyl thieno[3,2-c]pyridine-2-carboxylate (1.50 g, yield 54.9%) as a white solid. LC/MS (ESI) in/z: 194 (M+H)⁺.

### Step 2: Thieno[3,2-c]pyridin-2-ylmethanol

To a mixture of methyl thieno[3,2-c]pyridine-2-carboxylate (600 mg, 3.11 mmol) in THF (10 mL) was added LiAlH₄ (6.20 mL, 6.20 mmol, 1.0 M in THF) drop-wisely at room temperature under N₂ atmosphere. The mixture was stirred at room temperature for 2 hours. The mixture was quenched with Na₂SO₄.10H₂O at 0° C. and stirred at this temperature for 20 minutes. The mixture was filtered, and the filtrate was dried over anhydrous Na₂SO₄, filtered, and concentrated under reduced pressure to dryness. The residue was purified by flash chromatography (silica gel, 0-80% EtOAc in PE) to give thieno[3,2-c]pyridin-2-ylmethanol (300 mg, yield 58.5%) as a white solid. LC/MS (ESI) (m/z): 166 (M+H)⁺.

### Step 3: 2-(Azidomethyl)thieno[3,2-c]pyridine

To a solution of thieno[3,2-c]pyridin-2-ylmethanol (280 mg, 1.69 mmol) in toluene (3 mL) was added DBU (516 mg, 3.39 mmol) and DPPA (465 mg, 1.69 mmol) under N₂ atmosphere. The reaction mixture was stirred under N₂ atmosphere at 110° C. for 3 hours. The mixture was concentrated under reduced pressure to dryness. The residue was purified by prep-TLC (PE:EtOAc=1:1) to give 2-(azidomethyl)thieno[3,2-c]pyridine (120 mg, yield 37.3%) as a yellow oil. LC/MS (ESI) m/z: 191 (M+H)⁺.

### Step 4: Thieno[3,2-c]pyridin-2-ylmethanamine

To a solution of 2-(azidomethyl)thieno[3,2-c]pyridine (40 mg, 0.21 mmol) in THF/H₂O (1 mL, 1/1) was added PPh₃ (58 mg, 0.22 mmol) under N₂ atmosphere and the mixture was stirred at room temperature overnight. The mixture was concentrated under reduced pressure to dryness. The residue was purified by prep-TLC (DCM:MeOH=8:1) to give thieno[3,2-c]pyridin-2-ylmethanamine (30 mg, yield 87.0%) as a white solid. LC/MS (ESI) m/z: 165 (M+H)⁺.

### Step 5: GS)-N-((5-bromo-1H-indol-2-yl)methyl)-7-((phenoxathiine-3-carbonyl)glycyl)-1,4-dioxa-7-azaspiro[4.4]nonane-8-carboxamide (Compound 54)

To a mixture of (S)-7-((phenoxathiine-3-carbonyl)glycyl)-1,4-dioxa-7-azaspiro[4.4]nonane-8-carboxylic acid (15 mg, 0.033 mmol) and thieno[3,2-c]pyridin-2-ylmethanamine (11 mg, 0.066 mmol) in DMF (0.5 mL) was added DIPEA (26 mg, 0.2 mmol) and PyBOP (17 mg, 0.033 mmol) under N₂ atmosphere and the reaction mixture was stirred at room temperature for 1 hour. The mixture was diluted with EtOAc and washed with saturated aq. NaHCO₃ solution and brine, dried over anhydrous Na₂SO₄, filtered, and concentrated under reduced pressure to dryness. The residue was purified by prep-TLC (DCM:MeOH=7:1) and further purified by prep-HPLC to give Compound 54 (19.6 mg, yield 73.2%) as a white solid. ¹H NMR (400 MHz, CD₃OD): δ 8.92 (d, J=45.9 Hz, 1H), 8.17 (d, J=33.6 Hz, 1H), 7.82 (d, J=5.7 Hz, 1H), 7.48-7.41 (m, 2H), 7.35 (s, 1H), 7.20 (t, J=7.7 Hz, 1H), 7.10 (td, J=14.4, 7.7 Hz, 3H), 7.01 (d, J=8.1 Hz. 1H), 4.76 (s, 1H), 4.65 (dd, J=10.2, 4.8 Hz, 2H), 4.13 (dd, J=52.0, 16.4 Hz, 2H), 3.97 (d, J=25.8 Hz, 4H), 3.83 (q, J=10.8 Hz, 2H), 2.47 (dd, J=13.1, 9.3 Hz, 1H), 2.26 (dd, J=13.3, 5.3 Hz, 1H). LC/MS (ESI) m/z: 603 (M+H)⁺. RT (Method A): 1.42 min.

Compound 55 was prepared based on Scheme 19:

| Cmpd # | Reactant A | Reactant B | Characterization Data |
|---|---|---|---|
| 55^{a} | 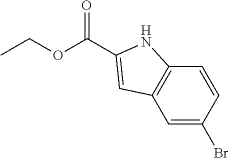 | 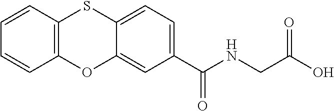 [SMILES: O=C(O)CNC(=O)c1ccc2c(c1)Oc1ccccc1S2] | ¹H NMR (400 MHz, CD₃OD): δ 7.51- 7.36 (m, 3H), 7.20-7.16 (m, 1H), 7.15-7.10 (m, 2H), 7.07 (dd, J = 10.4, 4.5 Hz, 2H), 7.03-6.96 (m, 2H), 6.30 (d, J = 31.6 Hz, 1H), 4.76-4.58 (m, 1H), 4.56 (d, J = 12.0 Hz, 2H), 4.11 (d, J = 11.1 Hz, 2H), 3.98 (d, J = 14.0 Hz, 4H), 3.78 (q, J = 10.6 Hz, 2H), 2.42 (dd, J = 13.2, 9.0 Hz, 1H), 2.25 (dd, J = 13.2, 6.2 Hz, 1H). LC/MS (ESI) m/z: 663 (M + H)⁺. RT (Method A): 2.65 min. |

^{a}Steps 2-5 only.

### Scheme 20. Synthesis of (1S,3S,5S)-N-(3-(1H-imidazol-1-yl)propyl)-5-methyl-2-((4-phenoxybutanoyl)glycyl)-2-azabicyclo[3.1.0]hexane-3-carboxamide (Compound 59)

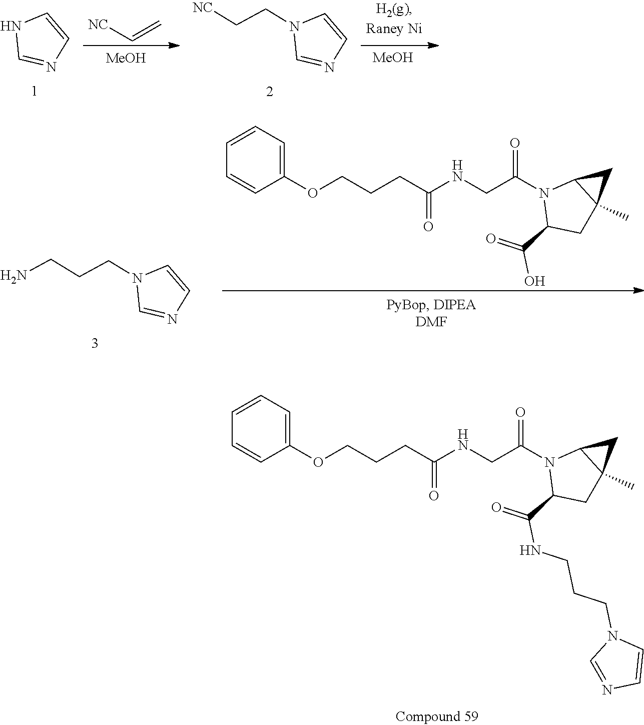

### Step 1: 3-(1H-imidazol-1-yl)propanenitrile

To a solution of 1H-imidazole (1.0 g, 14.71 mmol) in MeOH (5 mL) was added acrylonitrile (1.2 g, 22.07 mmol) at 0° C. and the reaction mixture was stirred at 55° C. 3 hours. The mixture was concentrated under reduced pressure to dryness. The residue was purified by flash chromatography (silica gel, 0-10% MeOH in DCM) to give 3-(1H-imidazol-1-yl)propanenitrile (1.70 g, yield 95.5%) as a brown solid. LC/MS (ESI) m/z: 122 (M III)⁺.

### Step 2: 3-(1H-imidazol-1-yl)propan-1-amine

To a solution of 3-(1H-imidazol-1-yl)propanenitrile (500 mg, 4.13 mmol) in MeOH (5 mL) was added Raney Ni (50 mg) under N₂ atmosphere. The mixture was degassed under N₂ atmosphere for three times and stirred under a H₂ balloon at room temperature overnight. The mixture was filtered, and filtrate was concentrated under reduced pressure to dryness to give 3-(1H-imidazol-1-yl)propan-1-amine (480 mg, yield 93.0%) as a colorless oil, which was used directly in the next step without further purification. LC/MS (ESI) (m/z): 126 (M+H)⁺.

### Step 3: (1S,3S,5S)-N-(3-(1H-imidazol-1-yl)propyl)-5-methyl-2-((4-phenoxybutanoyl) glycyl)-2-azabicyclo[3.1.0]hexane-3-carboxamide (Compound 59)

To a mixture of (1S,3S,5S)-5-methyl-2-((4-phenoxybutanoyl)glycyl)-2-azabicyclo [3.1.0]hexane-3-carboxylic acid (30 mg, 0.08 mmol) and 3-(1H-imidazol-1-yl)propan-1-amine (21 mg, 0.16 mmol) in DMF (3 mL) was added DIPEA (65 mg, 0.48 mmol) and PyBOP (48 mg, 0.09 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:McOH=10:1) and further purified by prep-HPLC to give Compound 59 (10 mg, yield 25.6%) as a colorless oil. ¹H NMR (400 MHz, CD₃OD): δ 8.26 (s, 1H), 8.19 (s, 1H), 7.36 (s, 1H), 7.23 (dd, J=8.6, 7.4 Hz, 3H), 6.91-6.85 (m, 3H), 4.73 (dd, J=11.4, 3.7 Hz, 1H), 4.16 (t, J=4.9 Hz, 2H), 4.14-4.05 (m, 2H), 3.99 (t, J=6.3 Hz, 2H), 3.22-3.16 (m, 1H), 3.13-3.04 (m, 1H), 2.47 (t, J=7.1 Hz, 2H), 2.38 (t, J=13.0 Hz, 1H), 2.12-1.96 (m, 5H), 1.27 (d, J=12.6 Hz, 4H), 1.19 (dd, J=5.5, 2.5 Hz, 1H), 0.83 (t, J=5.3 Hz, 1H). LC/MS (ESI) m/z: 468 (M+H)+. RT (Method A): 1.00 min.

The following compounds were prepared based on Scheme 20:

| Cmpd # | Reactant A | Reactant B | Characterization Data |
|---|---|---|---|
| 194^{a} | 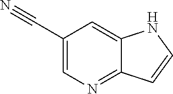 [SMILES: N#Cc1cnc2cc[nH]c2c1] | 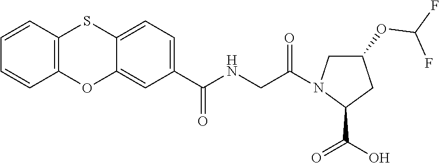 [SMILES: O=C(NCC(=O)N1C[C@H](OC(F)F)C[C@H]1C(=O)O)c1ccc2c(c1)Oc1ccccc1S2] | ¹H NMR (400 MHz, DMSO-d6): δ 11.22 (s, 1H), 8.88-8.52 (m, 2H), 8.26 (dd, J = 15.9, 1.8 Hz, 1H), 7.66 (d, J = 26.3 Hz, 1H), 7.61-7.55 (m, 3H), 7.37 (dd, J = 8.0, 4.1 Hz, 1H), 7.30-7.24 (m, 2H), 7.15-7.11 (m, 2H), 6.98-6.59 (m, 1H), 6.54-6.44 (m, 1H), 4.96-4.84 (m, 1H), 4.46-4.41 (m, 1H), 4.38 (d, J = 5.7 Hz, 1H), 4.20 (dd, J = 16.9, 5.9 Hz, 1H), 4.13-3.95 (m, 1H), 3.86 (dd, J = 11.3, 4.8 Hz, 1H), 3.76 (d, J = 12.9 Hz, 1H), 3.70-3.57 (m, 1H), 2.30-2.20 (m, 1H), 2.12-2.02 (m, 1H). LC/MS (ESI) m/z: 594 (M + H)⁺. RT (Method A): 1.53 min. |
| 222^{a,b} | 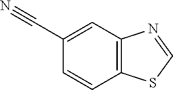 [SMILES: N#Cc1ccc2scnc2c1] | 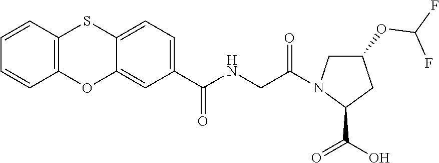 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 9.20 (d, J = 17.8 Hz, 1H), 8.04-7.94 (m, 2H), 7.51- 7.43 (m, 3H), 7.22-7.13 (m, 3H), 7.05 (dd, J = 12.7, 7.8 Hz, 2H), 6.50 (dd, J = 83.2, 65.8 Hz, 1H), 5.01 (s, 1H), 4.63-4.57 (m, 3H), 4.19 (dd, J = 40.7, 16.6 Hz, 2H), 4.00- 3.84 (m, 2H), 2.55-2.44 (m, 1H), 2.30-2.21 (m, 1H). LC/MS (ESI) m/z: 611 (M + H)⁺. RT (Method A): 2.20 min. |
| 227^{a,b} | 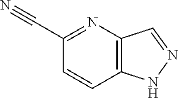 [SMILES: N#Cc1ccc2[nH]ncc2n1] | 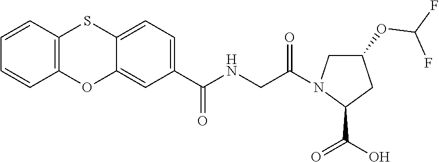 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 8.14 (s, 1H), 7.93 (d, J = 8.9 Hz, 1H), 7.51-7.45 (m, 3H), 7.22-7.14 (m, 3H), 7.06 (dd, J = 14.1, 6.4 Hz, 2H), 6.52 (t, J = 74.5 Hz, 1H), 5.04 (s, 1H), 4.64 (q, J = 5.7 Hz, 3H), 4.26-4.14 (m, 2H), 4.00 (dd, J = 11.4, 4.7 Hz, 1H), 3.90-3.85 (m, 1H), 2.54-2.47 (m, 1H), 2.35-2.27 (m, 1H). LC/MS (ESI) m/z: 595 (M + H)⁺. RT (Method A): 1.80 min. |
| 229^{a,b} | 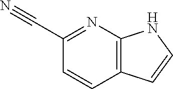 [SMILES: N#Cc1ccc2cc[nH]c2n1] | 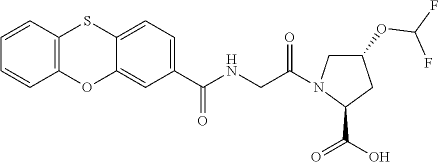 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 7.88 (d, J = 8.0 Hz, 1H), 7.52-7.49 (m, 1H), 7.47 (d, J = 1.8 Hz, 1H), 7.30 (d, J = 3.5 Hz, 1H), 7.20 (d, J = 1.6 Hz, 1H), 7.18-7.16 (m, 1H), 7.14-7.13 (m, 1H), 7.11-7.09 (m, 1H), 7.08-7.05 (m, 1H), 7.03 (dd, J = 8.1, 1.0 Hz, 1H), 6.53-6.42 (m, 1H), 6.40 (d, J = 3.5 Hz, 1H), 5.06-5.00 (m, 1H), 4.65 (t, J = 7.3 Hz, 1H), 4.58 (s, 2H), 4.26 (d, J = 16.7 Hz, 1H), 4.16 (d, J = 16.6 Hz, 1H), 4.00-3.96 (m, 1H), 3.86 (d, J = 12.8 Hz, 1H), 2.53-2.46 (m, 1H), 2.34-2.27 (m, 1H). LC/MS (ESI) m/z: 594 (M + H)⁺. RT (Method A): 2.10 min. |
| 230^{a,b} | 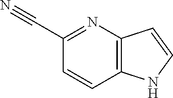 [SMILES: N#Cc1ccc2[nH]ccc2n1] | 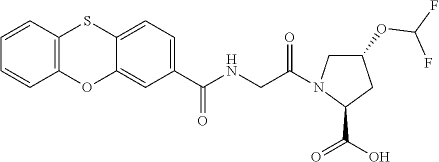 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 7.73 (d, J = 8.4 Hz, 1H), 7.54-7.45 (m, 3H), 7.22- 7.13 (m, 4H), 7.09-7.01 (m, 2H), 6.70-6.29 (m, 2H), 5.03 (s, 1H), 4.67-4.57 (m, 3H), 4.25 (d, J = 16.7 Hz, 1H), 4.15 (d, J = 16.6 Hz, 1H), 4.02-3.92 (m, 1H), 3.91- 3.84 (m, 1H), 2.54-2.41 (m, 1H), 2.33-2.16 (m, 1H). LC/MS (ESI) m/z: 594 (M + H)⁺. RT (Method A): 1.50 min. |
| 236^{a,b} | 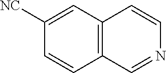 [SMILES: N#Cc1ccc2cnccc2c1] | 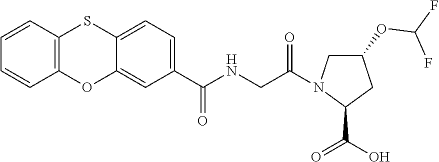 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 9.07 (s, 1H), 8.25 (d, J = 5.8 Hz, 1H), 7.97 (d, J = 8.6 Hz, 1H), 7.88 (s, 1H), 7.69 (d, J = 5.9 Hz, 1H), 7.61 (d, J = 8.6 Hz, 1H), 7.47 (d, J = 8.1 Hz, 1H), 7.38 (s, 1H), 7.22 (d, J = 7.1 Hz, 1H), 7.15 (d, J = 4.7 Hz, 2H), 7.10 (d, J = 7.3 Hz, 1H), 7.02 (d, J = 8.0 Hz, 1H), 6.53 (t, J = 74.5 Hz, 1H), 5.06-5.02 (m, 1H), 4.67-4.61 (m, 3H), 4.20 (s, 2H), 4.03 (dd, J = 11.4, 4.5 Hz, 1H), 3.89 (d, J = 11.2 Hz, 1H), 2.56-2.50 (m, 1H), 2.33-2.26 (m, 1H). LC/MS (ESI) m/z: 605 (M + H)⁺. RT (Method A): 1.61 min. |
| 237^{a,b} | 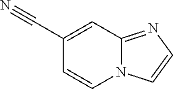 [SMILES: N#Cc1ccn2ccnc2c1] | 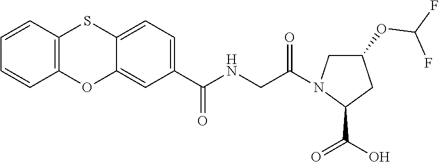 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 8.35-8.33 (m, 1H), 7.77-7.75 (m, 1H), 7.53-7.49 (m, 3H), 7.45 (d, J = 1.7 Hz, 1H), 7.21-7.15 (m, 3H), 7.11-7.08 (m, 1H), 7.05-7.02 (m, 1H), 6.94 (dd, J = 6.8, 1.0 Hz, 1H), 6.53 (t, J = 74.6 Hz, 1H), 5.05-5.01 (m, 1H), 4.62 (t, J = 7.8 Hz, 1H), 4.49 (s, 2H), 4.20 (d, J = 20.0 Hz, 2H), 4.01-3.97 (m, 1H), 3.91-3.86 (m, 1H), 2.55-2.48 (m, 1H), 2.31-2.23 (m, 1H). LC/MS (ESI) m/z: 594 (M + H)⁺. RT (Method A): 1.45 min. |
| 249^{a,b} | 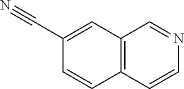 [SMILES: N#Cc1ccc2ccncc2c1] | 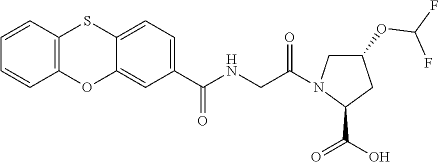 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 9.12 (s, 1H), 8.28 (d, J = 5.8 Hz, 1H), 7.98 (s, 1H), 7.81 (d, J = 8.6 Hz, 1H), 7.72 (dd, J = 8.6, 1.6 Hz, 1H), 7.66 (d, J = 5.8 Hz, 1H), 7.43 (dd, J = 8.1, 1.8 Hz, 1H), 7.32 (d, J = 1.8 Hz, 1H), 7.23-7.19 (m, 1H), 7.16-7.08 (m, 3H), 7.01 (dd, J = 8.1, 1.1 Hz, 1H), 6.53 (t, J = 74.5 Hz, 1H), 5.06-5.01 (m, 1H), 4.71- 4.58 (m, 3H), 4.19 (d, J = 3.7 Hz, 2H), 4.04 (dd, J = 11.5, 4.8 Hz, 1H), 3.92-3.88 (m, 1H), 2.57-2.50 (m, 1H), 2.33-2.26 (m, 1H). L.C/MS (ESI) m/z: 605 (M + H)⁺. RT (Method A): 1.63 min. |

^{a}Steps 2 and 3 only.
^{b}HATU used in place of PyBOP in Step 3.

### Scheme 21. Synthesis of (1S,3S,5S)-N-((R)-1-((1H-imidazol-4-yl)methyl)-2-oxopyrrolidin-3-yl)-5-methyl-2-((4-phenoxybutanoyl)glycyl)-2-azabicyclo[3.1.0]hexane-3-carboxamide (Compound 61)

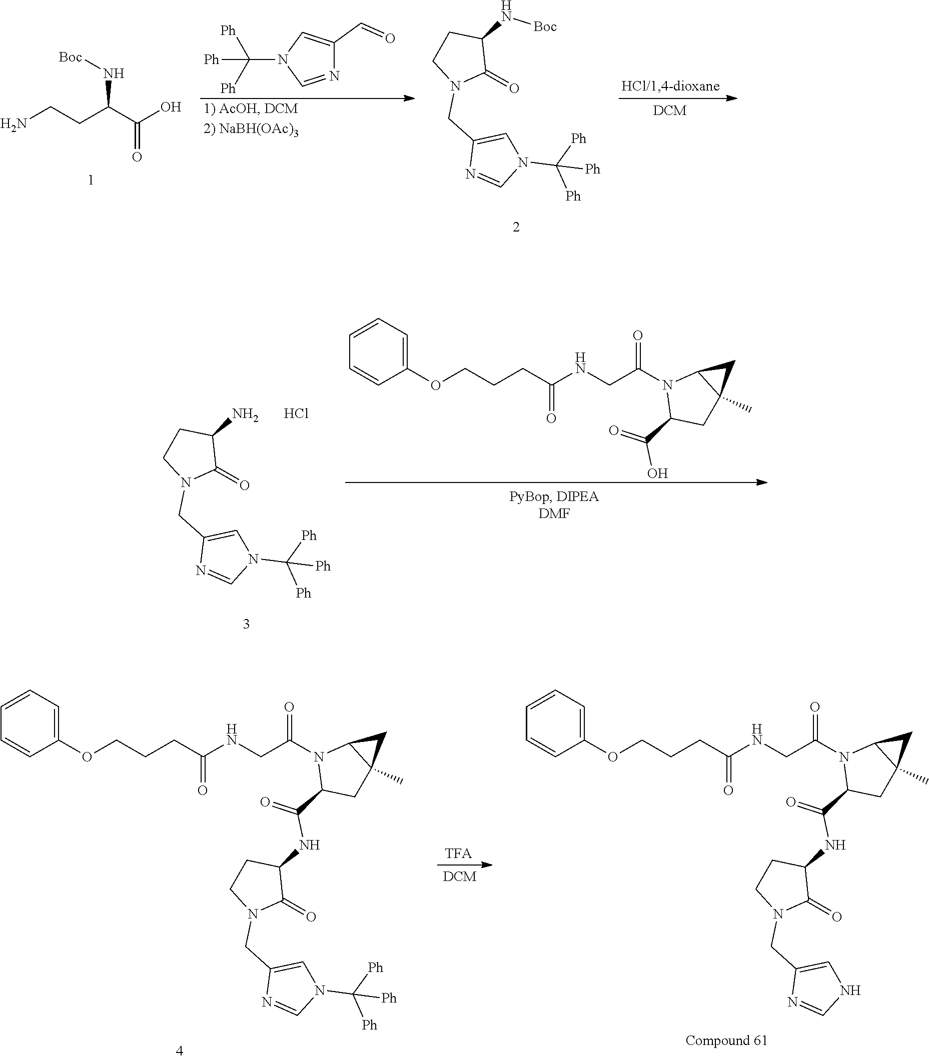

### Step 1: Tert-butyl (R)-(2-oxo-1-((1-trityl-1H-imidazol-4-ylmethyl)pyrrolidin-3-yl)carbamate (0.1)

To a mixture of (R)-4-amino-2-((tert-butoxy carbonyl)amino)butanoic acid (100 mg, 0.46 mmol) and 1-trityl-1H-imidazole-4-carbaldehyde (78 mg, 0.23 mmol) in DCM (2 mL) was added AcOII (0.2 mL) and the mixture was stirred at room temperature for 2 hours. And then, NaBH(OAc)₃ (292 mg, 1.38 mmol) was added to the above mixture in portions at 0° C. and the resulting mixture was stirred at room temperature overnight. The mixture was quenched with saturated aq. NH₄Cl solution and extracted with DCM twice. The combined organic layers were washed with brine, dried over anhydrous N₂SO₄, filtered, and concentrated under reduced pressure to dryness. The residue was purified by prep-TLC (PE:EtOAc=3:1) to give tert-butyl (R)-(2-oxo-1-((1-trityl-1H-imidazol-4-yl)methyl)pyrrolidin-3-yl)carbaminate (110 mg, yield 45.8%) as a white solid. LC/MS (ESI) m/z: 523 (M+H)⁺.

### Step 2: (R)-3-amino-1-((1-trityl-1H-imidazol-4-yl)methyl)pyrrolidin-2-one hydrochloride (2)

A solution of tert-butyl (R)-(2-oxo-1-((1-trityl-1H-imidazol-4-yl)methyl)pyrrolidin-3-yl)carbamate (110 mg, 0.21 mmol) in HCl/1,4-dioxane (1 mL, 4M) was stirred under N₂ atmosphere at room temperature for 2 hours. The reaction mixture was concentrated under reduced pressure to dryness to give (R)-3-amino-1-((1-trityl-LH-imidazol-4-yl)methyl)pyrrolidin-2-one hydrochloride (80 mg, yield 90.1%) as a white solid, which was used directly in the next step without further purification. LC/MS (ESI) m/z: 423 (M+H)⁺.

### Step 3: (1S,3S,5S)-5-methyl-N-((R)-2-oxo-1-((1-trityl-1H-imidazol-4-yl)methyl) pyrrolidin-3-yl)-2-((4-phenoxybutanoyl)glycyl)-2-azabicyclo[3.1.0]hexane-3-carboxamide

To a mixture of (1S,3S,5S)-5-methyl-2-((4-phenoxybutanoyl)glycyl)-2-azabicyclo[3.1.0]hexane-3-carboxylic acid (50 mg, 0.14 mmol) and (R)-3-amino-1-((1-trityl-1H-imidazol-4-yl)methyl)pyrrolidin-2-one hydrochloride (65 mg, 0.15 mmol) in DMF (2 mL) was added DIPEA (90 mg, 0.70 mmol) and PyBOP (94 mg, 0.18 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=10:1) to give (1S,3S,5S)-5-methyl-N-((R)-2-oxo-1-((1-trityl-1H-imidazol-4-yl)methyl)pyrrolidin-3-yl)-2-((4-phenoxybutanoyl)glycyl)-2-azabicyclo[3.1.0]hexane-3-carboxamide (50 mg, yield 46.7%) as a white solid. LC/MS (ESI) m/z: 765 (M+H)⁺.

### Step 4: (1S,3S,5S)-N-((R)-1-((1H-imidazol-4-yl)methyl)-2-oxopyrrolidin-3-yl)-5-methyl-2-((4-phenoxybutanoyl)glycyl)-2-azabicyclo[3.1.0]hexane-3-carboxamide (Compound 61)

To a solution of (1S,3S,5S)-5-methyl-N-((R)-2-oxo-1-((1-trityl-1H-imidazol-4-yl)methyl)pyrrolidin-3-yl)-2-((4-phenoxybutanoyl)glycyl)-2-azabicyclo[3.1.0]hexane-3-carboxamide (50 mg, 0.065 mmol) in DCM (2 mL) was added TFA (1 mL) and the mixture was stirred under N₂ atmosphere at room temperature for 2 hours. The reaction mixture was concentrated under reduced pressure to dryness. The residue was purified by prep-HPLC to give Compound 61 (5.3 mg, yield 15.6%) as a white solid. ¹H NMR (400 MHz, CD₃OD): δ 8.16 (s, 1H), 8.05 (d, J=11.5 Hz, 1H), 7.22 (d, J=3.3 Hz, 2H), 6.89 (dd, J=7.5, 4.4 Hz, 3H), 4.79 (dd, J=11.2, 3.3 Hz, 1H), 4.51-4.45 (m, 2H), 4.39 (t, J=9.4 Hz, 1H), 4.17 (d, J=17.1 Hz, 2H), 3.99 (t, J=6.3 Hz, 2H), 3.36-3.33 (m, 1H), 3.28 (d, J=9.5 Hz, 1H), 2.52-2.23 (m, 6H), 2.09-2.03 (m, 2H), 2.01-1.94 (m, 1H), 1.29 (d, J=9.9 Hz, 3H), 1.27-1.25 (m, 1H), 0.78 (t, J=5.7 Hz, 1H). LC/MS (ESI) (m/z): 523 (M+H)⁺. RT (Method A): 0.92 min.

### Scheme 22. Synthesis of (1S,3S,5S)-5-methyl-2-((4-phenoxybutanoyl)glycyl)-N-(1-(1,2,3,4-tetrahydroisoquinolin-7-yl)cyclopropyl)-2-azabicyclo[3.1.0]hexane-3-carboxamide (Compound 62)

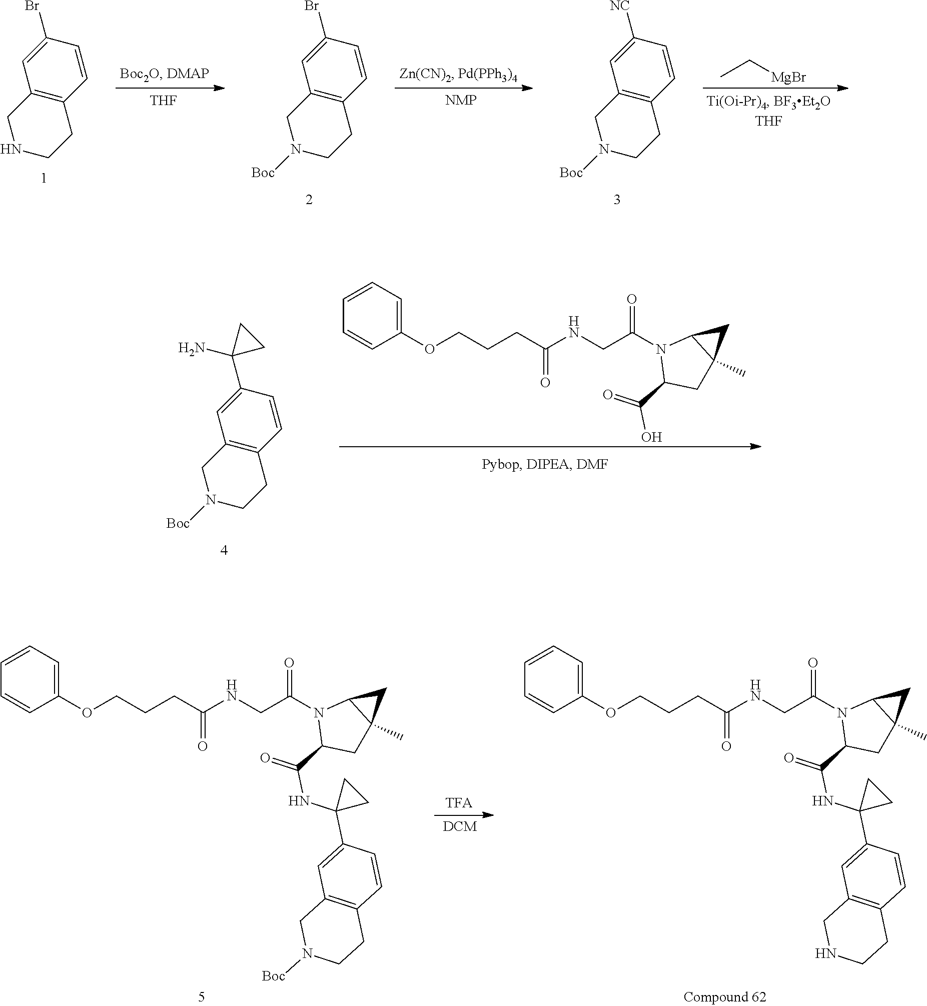

### Step 1: Tert-butyl 7-bromo-3,4-dihydroisoquinoline-2(1H)-carboxylate (2)

To a mixture of 7-bromo-1,2,3,4-tetmhydroisoquinoline (2 g, 9.43 mmol) and (Boc)₂O (4.1 g, 18.86 mmol) in THE (20 mL) was added 4-DMAP (58 mg, 0.47 mmol) and the mixture was stirred at room temperature for 6 hours. The mixture was concentered under reduced pressure to dryness. The residue was purified by flash chromatography (silica gel, 0-20% EtOAc in PE) to give tert-butyl 7-bromo-3,4-dihydroisoquinoline-2(1H)-carboxylate (2.1 g, yield 71.4%) as a white solid. LC/MS (ESI) m/z: 312 (M+H)⁺.

### Step 2: Tert-butyl 7-cyano-3,4-dihydroisoquinoline-2(1H)-carboxylate (3)

To a mixture of tert-butyl 7-bromo-3,4-dihydroisoquinoline-2(1H)-carboxylate (1.8 g, 5.79 mmol) and Zn(CN)₂ (1.36 g, 11.58 mmol) in NMP (18 mL) was added Pd(PPh₃)₄ (1.34 g, 1.16 mmol) under N₂ atmosphere, the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give tert-butyl 7-cyano-3,4-dihydroisoquinoline-2(1H)-carboxylate (1.12 g, yield 74.7%) as a white solid. ¹H NMR (400 MHz, CDCl₃) δ 7.43 (d, J=7.9 Hz, 1H), 7.40 (s, 1H), 7.23 (d, J=7.9 Hz, 1H), 4.58 (s, 2H), 3.65 (t, J=5.8 Hz, 2H), 2.88 (t, J=5.7 Hz, 2H), 1.48 (s, 9H).

### Step 3: Tert-butyl 7-(1-aminocyclopropyl)-3,4-dihydroisoquinoline-2(1H)-carboxylate (4)

To a mixture of tert-butyl 7-cyano-3,4-dihydroisoquinoline-2(1H)-carboxylate (300 mg, 1.16 mmol) and Ti(Oi-Pr)₄ (727 mg, 2.56 mmol) in anhydrous THF (3 mL) was added ethylmagnesium bromide (5.1 mL, 5.1 mmol, 1M in THF) drop-wisely under N₂ atmosphere at −70° C. for 1 hour and the reaction mixture was slowly warmed to 10° C. And then, to the reaction mixture was added BF₃.Et₂O (330 mg, 2.33 mmol) and the resulting mixture was stirred at 10° C. for 1 hour. The mixture was quenched with 1N aq. HCl, basified with 2N aq. NaOH solution to pH˜10 and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give tert-butyl 7-(1-aminocyclopropyl)-3,4-dihydroisoquinoline-2(1H)-carboxylate (21 mg, yield 6.3%) as a colorless oil. LC/MS (ESI) m/z: 289 (M+H)⁺.

### Step 4: Tert-butyl 7-(1-((1S,3S,5S)-5-methyl-2-((4-phenoxybutanoyl)glycyl)-2-azabicyclo[3.1.0]hexane-3-carboxamido)cyclopropyl)-3,4-dihydroisoqmmoline-2(1H)-carboxylate (5)

To a mixture of tert-butyl 7-(1-aminocyclopropyl)-3,4-dihydroisoquinoline-2(1H)-carboxylate (21 mg, 0.073 mmol) and (1S,3S,5S)-5-methyl-2-((4-phenoxybutanoyl) glycyl)-2-azabicyclo[3.1.0]hexane-3-carboxylic acid (29 mg, 0.08 mmol) in DMF (1 mL) was added DIPEA (47 mg, 0.37 mmol) and PyBOP (49 mg, 0.095 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=20:1) to give tert-butyl 7-(1-((1S,3S,5S)-5-methyl-2-((4-phenoxybutanoyl)glycyl)-2-azabicyclo[3.1.0]hexane-3-carboxamido)cyclopropyl)-3,4-dihydroisoquinoline-2(1H)-carboxylate (20 mg, yield 43.5%) as a white solid. LC/MS (ESI) m/z: 631 (M+H)⁺.

### Step 5: (1S,3S,5S)-5-methyl-2-((4-phenoxybutanoyl)glycyl)-N-(1-(1,2,3,4-tetrahydroisoquinolin-7-yl)cyclopropyl)-2-azabicyclo[3.1.0]hexane-3-carboxamide (Compound 62)

To a solution of tert-butyl 7-(1-((1S,3S,5S)-5-methyl-2-((4-phenoxybutanoyl)glycyl)-2-azabicyclo[3.1.0]hexane-3-carboxamido)cyclopropyl)-3,4-dihydroisoquinoline-2(1H)-carboxylate (20 mg, 0.032 mmol) in DCM (1 mL) was added TFA (0.5 mL) and the mixture was stirred under N₂ atmosphere at room temperature for 2 hours. The reaction mixture was concentrated under reduced pressure to dryness. The residue was purified by prep-HPLC to give Compound 62 (2.9 mg, yield 17.1%) as a white solid. ¹H NMR (400 MHz, CD₃OD): δ 8.48 (s, 1H), 7.24 (t, J=7.8 Hz, 2H), 7.10 (dd, J=19.4, 9.3 Hz, 3H), 6.90 (dd, J=7.5, 4.7 Hz, 3H), 4.77 (dd, J=11.4, 3.4 Hz, 1H), 4.29 (s, 2H), 4.24 (d, J=16.8 Hz, 1H), 4.09 (d, J=16.6 Hz, 1H), 4.01 (t, J=6.3 Hz, 2H), 3.43 (t, J=6.0 Hz, 2H), 3.03 (t, J=6.1 Hz, 2H), 2.49 (t, J=7.5 Hz, 2H), 2.38 (t, J=12.3 Hz, 1H), 2.11-2.04 (m, 3H), 1.28 (s, 3H), 1.25-1.13 (m, 6H), 0.78 (t, J=5.5 Hz, 1H). LC/MS (ESI) (m/z): 531 (M+H)⁺. RT (Method A): 1.25 min.

### Scheme 23. Synthesis of (1S,3S,5S)-N-((8-(aminomethyl)imidazo[1,2-a]pyridin-3-yl)methyl)-5-methyl-2-((4-phenoxybutanoyl)glycyl)-2-azabicyclo[3.1.0]hexane-3-carboxamide (Compound 63)

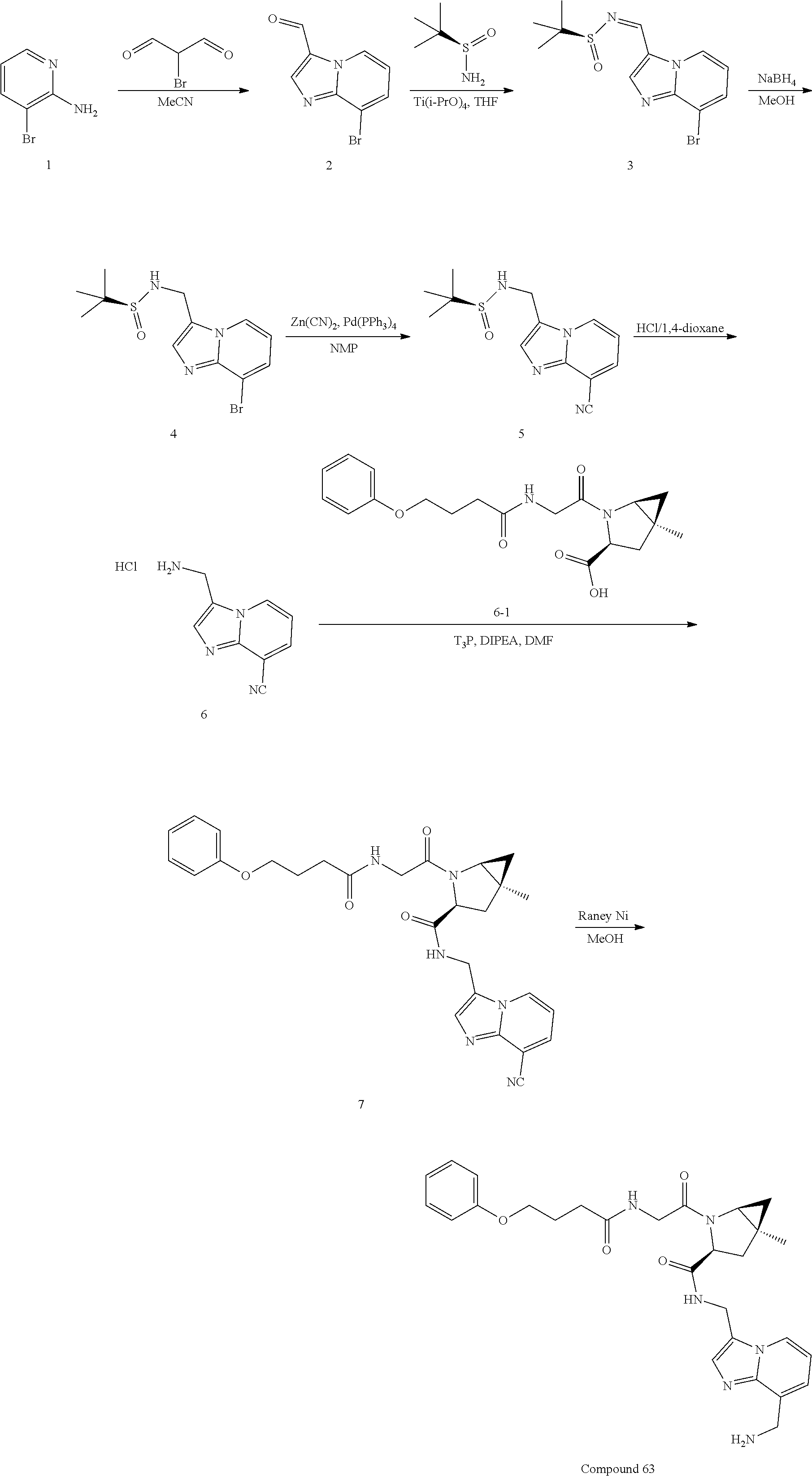

### Step 1: 8-Bromoimidazo[1,2-a]pyridine-3-carbaldehyde (2)

To a solution of 3-bromopyridin-2-amine (2.0 g, 11.56 mmol) in MeCN (30 mL) was added 2-bromomalonaldehyde (2.1 g, 13.87 mmol) under N₂ atmosphere, the mixture was degassed under N₂ atmosphere for three times and stirred at 85° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 20-50% EtOAc in PE) to give 8-bromoimidazo[1,2-a]pyridine-3-carbaldehyde (1.53 g, yield 58.8%) as a brown solid. LC/MS (ESI) m/z: 225 (M+H)⁺.

### Step 2: (Z)-N-((8-bromoimidazo[1,2-a]pyridin-3-yl)methylene)-2-methylpropane-2-sulfinamide (3)

To a solution of 8-bromoimidazo[1,2-a]pyridine-3-carbaldehyde (1.53 g, 6.80 mmol) in THF (30 mL) was added (R)-2-methylpropane-2-sulfinamide (2.5 g, 20.40 mmol) and Ti(i-PrO)₄ (9.7 g, 33.99 mmol), the mixture was degassed under N₂ atmosphere for three times and stirred at 90° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-40% EtOAc in PE) to give (Z)-N-((8-bromoimidazo[1,2-a]pyridin-3-yl)methylene)-2-methylpropane-2-sulfinamide (1.95 g, yield 87.4%) as a brown solid. LC/MS (ESI) m/z: 328 (M+H)⁺.

### Step 3: N-((8-bromoimidazo[1,2-a]pyridin-3-yl)methyl)-2-methylpropane-2-sulfinamide (4)

To a solution of (Z)-N-((8-bromoimidazo[1,2-a]pyridin-3-yl)methylene)-2-methylpropane-2-sulfinamide (1.95 g, 5.94 mmol) in MeOH (30 mL) was added NaBH₄ (449 mg, 11.88 mmol) in portions under N₂ atmosphere and the reaction mixture was stirred at room temperature for 2 hours. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-40% EtOAc in PE) to give N-((8-bromoimidazo [1,2-a]pyridin-3-yl)methyl)-2-methylpropane-2-sulfinamide (1.96 g, yield 99.9%) as a brown solid. LC/MS (ESI) m/z: 330 (M+H)⁺.

### Step 4: N-((8-cyanoimidazo[1,2-a]pyridin-3-yl)methyl)-2-methylpropane-2-sulfinamide (5)

To a solution of N-((8-bromoimidazo[1,2-a]pyridin-3-yl)methyl)-2-methylpropane-2-sulfinamide (1.56 g, 4.72 mmol) in NMP (20 mL) was added Zn(CN)₂ (1.1 g, 9.45 mmol) and Pd(PPh₃)₄ (819 mg, 0.71 mmol) under N₂ atmosphere and the reaction mixture was stirred at 100° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give N-((8-cyanoimidazo[1,2-a]pyridin-3-yl)methyl)-2-methylpropane-2-sulfinamide (200 mg, yield 15.3%) as a white solid. LC/MS (ESI) m/z: 277 (M+H)⁺.

### Step 5: 3-(Aminomethyl)imidazo[1,2-a]pyridine-8-carbonitrile hydrochloride (6)

A solution of N-((8-cyanoimidazo[1,2-a]pyridin-3-yl)methyl)-2-methylpropane-2-sulfinamide (200 mg, 0.72 mmol) in HCl/1,4-dioxane (2 mL, 4M) was stirred under N₂ atmosphere at room temperature for 2 hours. The reaction mixture was concentrated under reduced pressure to dryness to give 3-(aminomethyl)imidazo[1,2-a]pyridine-8-carbonitrile hydrochloride (150 mg, yield 99.4%) as a yellow solid, which was used directly in the next step without further purification. LC/MS (ESI) m/z: 173 (M+H)⁺.

### Step 6: (1S,3S,5S)-N-((8-cyanoimidazo[1,2-a]pyridin-3-yl)methyl)-5-methyl-2-((4-phenoxybutanoyl)glycyl)-2-azabicyclo[3.1.0]hexane-3-carboxamide (7)

To a mixture of (1S,3S,5S)-5-methyl-2-((4-phenoxybutanoyl)glycyl)-2-azabicyclo[3.1.0]hexane-3-carboxylic acid (30 mg, 0.083 mmol) and 3-(aminomethyl)imidazo[1,2-a]pyridine-8-carbonitrile hydrochloride (26 mg, 0.12 mmol) in DMF (5 mL) was added DIPEA (65 mg, 0.50 mmol) and PyBOP (65 mg, 0.12 mmol) under N₂ atmosphere and the mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-5% MeOH in DCM) to give (1S,3S,5S)-N-((8-cyanoimidazo[1,2-a]pyridin-3-yl)methyl)-5-methyl-2-((4-phenoxybutanoyl) glycyl)-2-azabicyclo[3.1.0]hexane-3-carboxamide (40 mg, yield 93.4%) as a white solid. LC/MS (ESI) m/z: 515 (M+H)⁺.

### Step 7: (1S,3S,5S)-N-((8-(aminomethyl)imidazo[1,2-a]pyridin-3-yl)methyl)-5-methyl-2-((4-phenoxybutanoyl)glycyl)-2-azabicyclo[3.1.0]hexane-3-carboxamide (Compound 63)

To a solution of (1S,3S,5S)-N-((8-cyanoimidazo[1,2-a]pyridin-3-yl)methyl)-5-methyl-2-((4-phenoxybutanoyl)glycyl)-2-azabicyclo[3.1.0]hexane-3-carboxamide (40 mg, 0.078 mmol) in MeOH (3 mL) was added Raney Ni (10 Ing, wet), the mixture was degassed under N₂ atmosphere for three times and stirred under a H₂ balloon at 25° C. overnight. The mixture was filtered, and the filtrate was concentrated to dryness. The residue was purified by prep-TLC (DCM:MeOH=5:1) and further purified by prep-HPLC to give Compound 63 (3.2 mg, yield 7.9%) as a white solid. ¹H NMR (400 MHz, CD₃OD): δ 8.43 (d, J=6.8 Hz, 1H), 8.39 (s, 1H), 7.63 (d, J=9.1 Hz, 1H), 7.39 (d, J=6.9 Hz, 1H), 7.23 (dd, J=10.3, 5.7 Hz, 2H), 7.02 (t, J=6.9 Hz, 1H), 6.89 (dd, J=7.4, 4.8 Hz, 3H), 4.76 (t, J=10.0 Hz, 2H), 4.69 (d, J=14.6 Hz, 1H), 4.43 (s, 2H), 4.15 (dd, J=38.0, 16.6 Hz, 2H), 4.01 (t, J=6.3 Hz, 2H), 3.29 (d, J=2.5 Hz, 1H), 2.47 (t, J=7.4 Hz, 2H), 2.35 (t, J=12.4 Hz, 1H), 2.12-2.03 (m, 3H), 1.27-1.20 (m, 3H), 1.13-0.91 (m, 1H), 0.76 (t, J=5.5 Hz, 1H). LC/MS (ESI) m/z: 519 (M+H)⁺. RT (Method A): 0.88 min.

Compound 78 was prepared based on Scheme 23:

| Cmpd # | Reactant A | Reactant B | Characterization Data |
|---|---|---|---|
| 78^{a,b} | 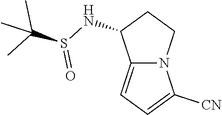 | 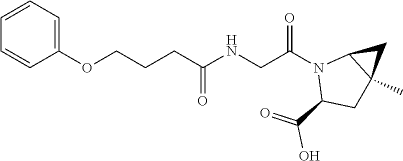 | ¹H NMR (400 MHz, CD₃OD): δ 7.29- 7.22 (m, 2H), 6.91 (d, J = 8.3 Hz, 3H), 6.29 (d, J = 3.6 Hz, 1H), 5.96- 5.90 (m, 1H), 5.28- 5.19 (m, 1H), 4.81-4.70 (m, 1H), 4.26-4.05 (m, 5H), 4.03-3.94 (m, 3H), 3.30-3.18 (m, 1H), 2.93-2.81 (m, 1H), 2.49- 2.43 (m, 2H), 2.37- 2.32 (m, 1H), 2.18-2.01 (m, 3H), 1.30-1.24 (m, 3H), 1.20-0.98 (m, 1H), 0.80-0.67 (m, 1H). LC/MS (ESI) m/z: 494 (M + H)⁺. RT (Method A): 1.03 min. |

^{a}Steps 5-7 only.
^{b}PyBOP used in place of T₃P.

### Scheme 24. Synthesis of (1S,3S,5S)-N-(1-(3-(aminomethyl)-5-methylphenyl) cyclopropyl)-5-methyl-2-((4-phenoxybutanoyl)glycyl)-2-azabicyclo[3.1.0]hexane-3-carboxamide (Compound 64)

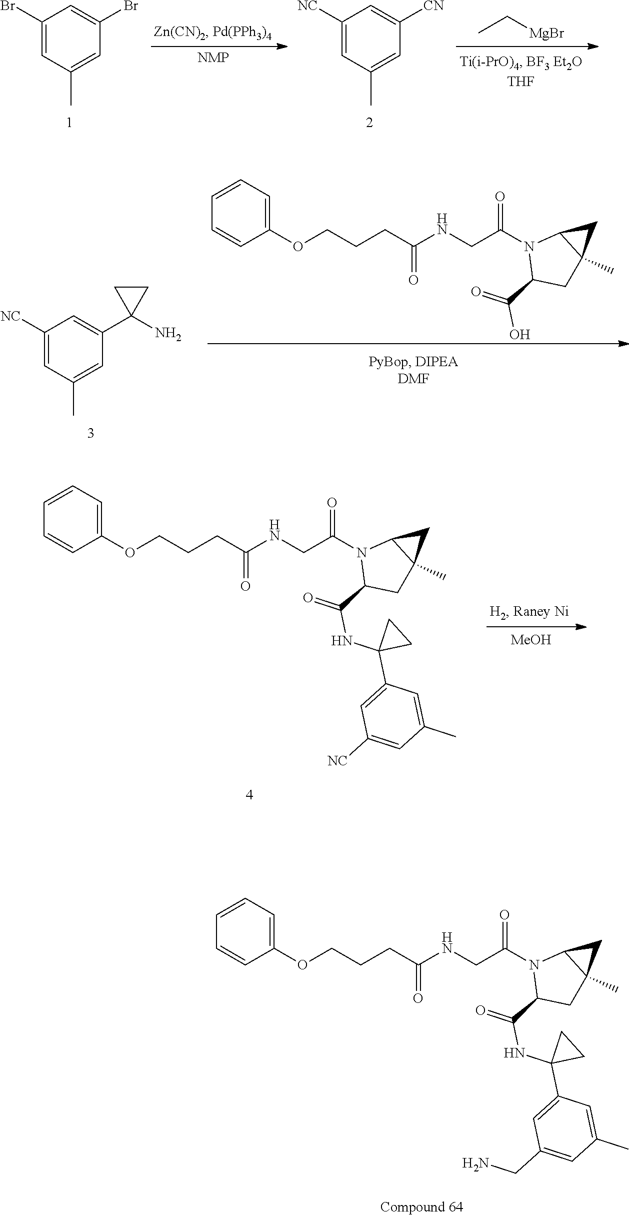

### Step 1: 5-Methylisophthalonitrile (2)

To a solution of 1,3-dibromo-5-methylbenzene (1.0 g, 4.00 mmol) in NMP (20 mL) was added Zn(CN)₂ (940 mg, 8.00 mmol) and Pd(PPh₃)₄ (694 mg, 0.60 mmol) under N₂ atmosphere and the reaction mixture was stirred at 110° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give 5-methylisophthalonitrile (460 mg, yield 80.9%) as a white solid.

### Step 2: 3-(1-Aminocyclopropyl)-5-methylbenzonitrile (3)

To a mixture of 5-methylisophthalonitrile (200 mg, 1.41 mmol) and Ti(i-PrO)₄ (440 mg, 1.55 mmol) in THE (5 mL) was added ethylmagnesium bromide (3.1 mL, 3.1 mmol, 1M in THF) drop-wisely at −70° C. under N₂ atmosphere for 1 hour and the reaction mixture was slowly warmed to 10° C. Then BF₃·Et₂O (399 mg, 2.81 mmol) was added into the above mixture and the resulting mixture was stirred at room temperature for 1 hour. The mixture was quenched with 1N aq. HCl, basified with 2N aq. NaOH solution to pH˜10 and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give 3-(1-aminocyclopropyl)-5-methylbenzonitrile (30 mg, yield 12.4%) as a white solid. LC/MS (ESI) m/z: 173 (M+H)⁺.

### Step 3: (1S,3S,5S)-N-(I-(3-cyano-5-methylphenyl)cyclopropyl)-5-methyl-2-((4-phenoxybutanoyl)glycyl)-2-azabicyclo[3.1.0]hexane-3-carboxamide (4)

To a mixture of (1S,3S,5S)-5-methyl-2-((4-phenoxybutanoyl)glycyl)-2-azabicyclo[3.1.0]hexane-3-carboxylic acid (30 mg, 0.083 mmol) and 3-(1-aminocyclopropyl)-5-methylbenzonitrile (22 mg, 0.12 mmol) in DMF (5 mL) was added DIPEA (65 mg, 0.50 mmol) and PyBOP (65 mg, 0.12 mmol) under N₂ atmosphere and the mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-5% MeOH in DCM) to give (1S,3S,5S)-N-(1-(3-cyano-5-methylphenyl)cyclopropyl)-5-methyl-2-((4-phenoxybutanoyl)glycyl)-2-azabicyclo[3.1.0]hexane-3-carboxamide (40 mg, yield 93.4%) as a white solid. LC/MS (ESI) m/z: 515 (M+H)⁺.

### Step 4: (1S,3S,5S)-N-(1-(3-(aminomethyl)-5-methylphenyl)cyclopropyl)-5-methyl-2-((4-phenoxybutanoyl)glycyl)-2-azabicyclo[3.1.0]hexane-3-carboxamide (Compound 64)

To a solution of (1S,3S,5S)-N-(1-(3-cyano-5-methylphenyl)cyclopropyl)-5-methyl-2-((4-phenoxybutanoyl)glycyl)-2-azabicyclo[3.1.0]hexane-3-carboxamide (40 mg, 0.078 mmol) in MeOH (3 mL) was added Raney Ni (10 mg, wet), the mixture was degassed under N₂ atmosphere for three times and stirred under a H₂ balloon at 25° C. overnight. The mixture was filtered, and the filtrate was concentrated to dryness. The residue was purified by prep-TLC (DCM:MeOH=5:1) and further purified by prep-HPLC to give Compound 64 (10 mg, yield 24.8%) as a white solid. ¹H NMR (400 MHz, CD₃OD) S 8.54 (s, 1H), 7.27-7.18 (m, 3H), 7.11-7.01 (m, 1H), 6.94 (s, 1H), 6.89 (d, J=8.5 Hz, 3H), 4.80 (d, J=7.9 Hz, 1H), 4.24 (d, J=16.5 Hz, 1H), 4.11 (d, J=16.5 Hz, 1H), 4.01 (dd, J=10.8, 5.2 Hz, 4H), 3.34 (d, J=2.4 Hz, 1H), 2.44 (dt, J=24.9, 9.5 Hz, 3H), 2.33 (s, 3H), 2.07 (dt, J=7.5, 4.9 Hz, 3H), 1.28 (s. 3H), 1.22 (dt, J=5.5, 4.5 Hz, 5H), 0.80 (t, J=5.2 Hz, 1H). LC/MS (ESI) m/z: 519 (M+H)⁺. RT (Method A): 1.30 min.

### Scheme 25. Synthesis of (1S,3S,5S)-5-methyl-N-(1-(3-methyl-3H-imidazo[4,5-c]pyridin-2-yl)cyclopropyl)-2-((4-phenoxybutanoyl)glycyl)-2-azabicyclo[3.1.0]hexane-3-carboxamide (Compound 65)

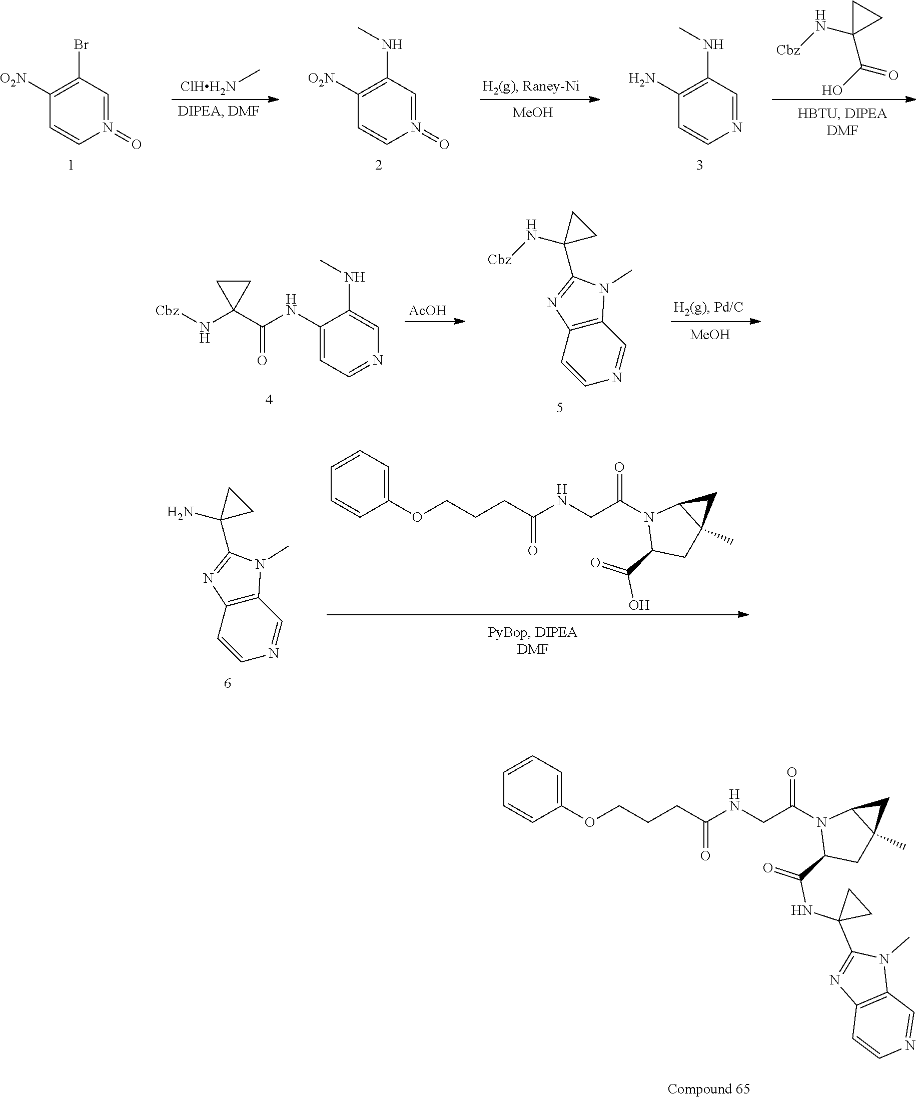

### Step 1: 3-(Methylamino)-4-nitropyridine 1-oxide

To a solution of 3-bromo-4-nitropyridine 1-oxide (2.0 g, 9.13 mmol) and methanamine hydrochloride (925 mg, 13.70 mmol) in DMF (20 mL) was added DIPEA (3.78 g, 27.39 mmol) at 0° C. and the mixture was stirred at 28° C. for 16 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-5% MeOH in DCM) to give 3-(methylamino)-4-nitropyridine 1-oxide (885 mg, yield 55.3%) as a yellow solid. LC/MS (ESI) n/z: 170 (M+H)⁺.

### Step 2: N³-methylpyridine-3,4-diamine

To a solution of 3-(methylamino)-4-nitropyridine 1-oxide (885 mg, 5.24 mmol) in MeOH (10 mL) was added Raney Ni (80 mg, wet), the mixture was degassed under N₂ atmosphere for three times and the mixture was stirred under a H₂ balloon at 25° C. for 16 hours. The mixture was filtered, and filtrate was concentrated under reduced pressure to dryness to give N³-methylpyridine-3,4-diamine (631 mg, yield 98.0%) as a brown oil, which was used directly in the next step without further purification. LC/MS (ESI) (m/z): 124 (M+H)⁺.

### Step 3: Benzyl (1-((3-(methylamino)pyridin-4-yl)carbamoyl)cyclopropyl)carbamate

To a mixture of N³-methylpyridine-3,4-diamine (631 mg, 5.13 mmol) and 1-(((benzyloxy)carbonyl)amino)cyclopropane-1-carboxylic acid (1.2 g, 5.13 mmol) in DMF (10 mL) was added DIPEA (3.97 g, 30.78 mmol) and HBTU (2.43 g, 6.41 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-5% MeOH in DCM) to give benzyl (1-((3-(methylamino)pyridin-4-yl)carbamoyl) cyclopropyl)carbamate (1.2 g, yield 68.8%) as gray solid. LC/MS (EST) m/z: 341 (M+H)⁺.

### Step 4: Benzyl (1-(3-methyl-3H-imidazo[4,5-c]pyridin-2-yl)cyclopropyl)carbamate

To a solution of benzyl (1-((3-(methylamino)pyridin-4-yl)carbamoyl)cyclopropyl) carbamate (600 mg, 1.76 mmol) in AcOH (5 mL) was stirred at 120° C. for 1.5 hours in a CEM microwave reactor. The mixture was diluted with EtOAc, washed with saturated aq. NaHCO₃ solution and brine, dried over anhydrous Na₂SO₄, filtered, and concentrated under reduced pressure to dryness to give benzyl (1-(3-methyl-3H-imidazo[4,5-c]pyridin-2-yl)cyclopropyl)carbamate (495 mg, yield 87.1%) as a light-yellow solid, which was used directly in the next step without further purification. LC/MS (ESI) m/z: 323 (M+H)⁺.

### Step 5: 1-(3-Methyl-3H-imidazo[4,5-c]pyridin-2-yl)cyclopropan-1-amine

To a solution of benzyl (1-(3-methyl-3H-imidazo[4,5-c]pyridin-2-yl)cyclopropyl) carbamate (250 mg, 0.78 mmol) in MeOH (5 mL) was added Pd/C (20 mg, 10% wt.), the mixture was degassed under N₂ atmosphere for three times and stirred under a H₂ balloon at 25° C. 1 hour. The mixture was filtered, and the filtrate was concentrated under reduced pressure to dryness to give 1-(3-methyl-3H-imidazo[4,5-c]pyridin-2-yl)cyclopropan-1-amine (118 mg, yield 80.8%) as a yellow solid, which was used directly in the next step without further purification. LC/MS (ESI) m/z: 189 (M+H)⁺.

### Step 6: (1S,3S,5S)-5-methyl-N-(1-(3-methyl-3H-imidazo[4,5-c]pyridin-2-yl) cyclopropyl)-2-((4-phenoxybutanoyl)glycyl)-2-azabicyclo[3.1.0]hexane-3-carboxamide (Compound 65)

To a mixture of (1S,3S,5S)-5-methyl-2-((4-phenoxybutanoyl)glycyl)-2-azabicyclo[3.1.0]hexane-3-carboxylic acid (30 mg, 0.08 mmol) and 1-(3-methyl-3H-imidazo[4,5-c]pyridin-2-yl)cyclopropan-1-amine (15 mg, 0.08 mmol) in DMF (1 mL) was added DIPEA (65 mg, 0.48 mmol) and PyBOP (48 mg, 0.09 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=10:1) and further purified by prep-HPLC to give Compound 65 (12 mg, yield 27.3%) as a white solid. ¹H NMR (400 MHz, CD₃OD): δ 8.81 (s, 1H), 8.31 (d, J=5.8 Hz, 1H), 8.17 (s, 1H), 7.64 (d, J=5.8 Hz, 1H), 7.26-7.20 (m, 2H), 6.88 (t, J=8.5 Hz, 3H), 4.73 (dd, J=11.4, 3.5 Hz, 1H), 4.22 (t, J=14.4 Hz, 1H), 4.05 (s, 3H), 4.03-3.95 (m, 3H), 3.26 (dd, J=5.9, 2.4 Hz, 1H), 2.40 (dt, J=23.7, 9.6 Hz, 3H), 2.05 (ddd, J=13.4, 12.1, 5.0 Hz, 3H), 1.80-1.70 (m, 1H), 1.56-1.46 (m, 1H), 1.41-1.31 (m, 2H), 1.25 (s, 3H), 1.11 (dd, J=5.6, 2.4 Hz, 1H), 0.75 (t, J=5.8 Hz, 1H). LC/MS (ESI) m/z: 531 (M+H)⁺. RT (Method A): 1.07 min.

Compound 109 was prepared based on Scheme 25:

| Cmpd # | Reactant A | Reactant B | Reactant C | Characterization Data |
|---|---|---|---|---|
| 109 | 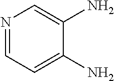 | 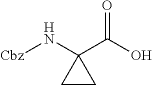 [SMILES: O=C(NC1(C(=O)O)CC1)OCc1ccccc1] | 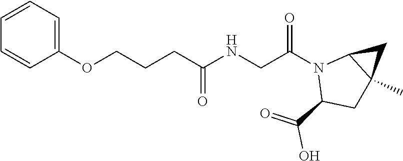 | ¹H NMR (400 MHz, CD₃OD): δ 8.73 (s, 1H), 8.21 (d, J = 5.6 Hz, 1H), 7.53 (dd, J = 5.7, 0.8 Hz, 1H), 7.18 (dd, J = 8.8, 7.2 Hz, 2H), 6.87-6.82 (m, 3H), 4.82 (d, J = 4.5 Hz, 1H), 4.36 (d, J = 16.9 Hz, 1H), 4.11 (d, J = 16.9 Hz, 1H), 4.01 (td, J = 6.3, 1.3 Hz, 2H), 3.35 (dd, J = 5.9, 2.3 Hz, 1H), 2.49 (ddd, J = 31.3, 15.8, 8.6 Hz, 3H), 2.17- 2.08 (m, 3H), 1.71 (tt, J = 11.3, 5.5 Hz, 2H), 1.43 (d, J = 2.8 Hz, 2H), 1.31 (s, 4H), 0.88 (t, J = 5.2 Hz, 1H). LC/MS (ESI) m/z: 517 (M + H)⁺. RT (Method A): 1.29 min. |

### Scheme 26. Synthesis of (2,3-dihydro-1H-pyrrolo[3,4-c]pyridin-6-yl)methyl (1S,3S,5S)-5-methyl-2-((4-phenoxybutanoyl)glycyl)-2-azabicyclo[3.1.0]hexane-3-carboxylate (Compound 66)

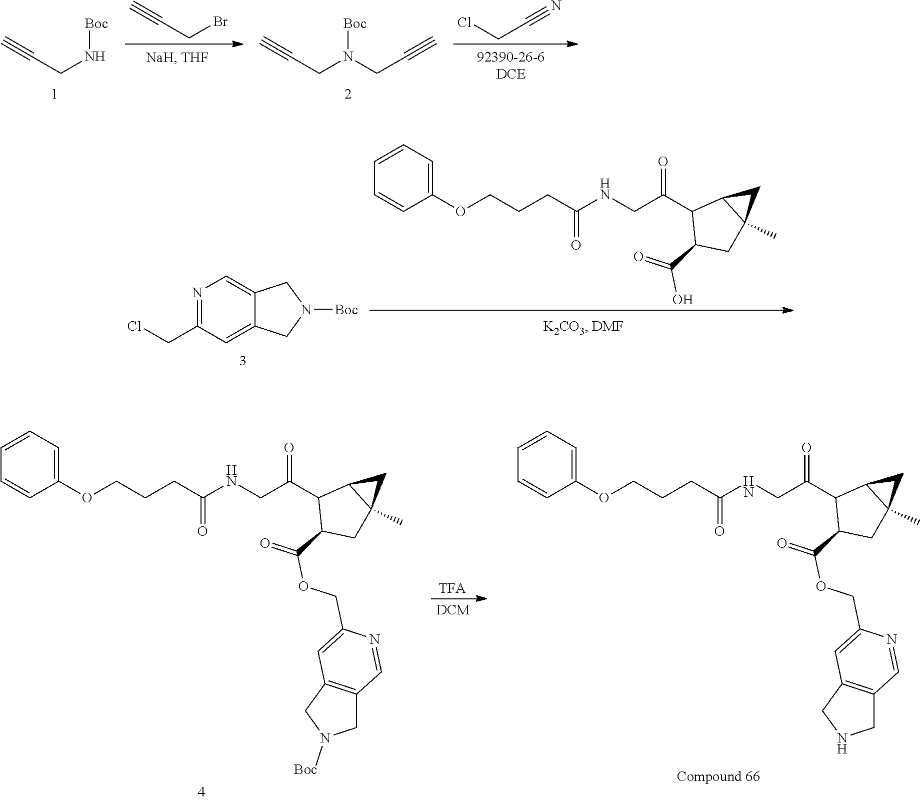

### Step 1: Tert-butyl di(prop-2-yn-1-yl)carbamate (2)

To a solution of tert-butyl prop-2-yn-1-ylcarbamate (1.0 g, 6.45 mmol) in THF (15 mL) was added NaH (310 mg, 7.74 mmol, 60% dispersion in mineral oil) and 3-bromoprop-1-yne (921 mg, 7.74 mmol) at 0° C. under N₂ atmosphere and the reaction mixture was stirred at room temperature overnight. The reaction mixture was quenched with saturated aq. NH₄Cl solution at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give tert-butyl di(prop-2-yn-1-yl)carbamate (700 mg, yield 56.2%) as a colorless oil.

### Step 2: Tert-butyl 6-(chloromethyl)-1,3-dihydro-2H-pyrrolo[3,4-c]pyridine-2-carboxylate (3)

To a mixture of tert-butyl di(prop-2-yn-1-yl)carbamate (600 mg, 3.11 mmol) and 2-chloroacetonitrile (282 mg, 3.73 mmol) in DCE (8 mL) was added 1,5-Cyclooctadiene, ruthenium complex (50 mg, 0.16 mmol) under N₂ atmosphere, the reaction mixture was degassed under N₂ atmosphere and stirred at 60° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give tert-butyl 6-(chloromethyl)-1,3-dihydro-2H-pyrrolo[3,4-c]pyridine-2-carboxylate (250 mg, yield 30.0%) as a white solid. LC/MS (ESI) m/z: 269 (M+H)⁺.

### Step 3: Tert-butyl 6-((((1S,3S,5S)-5-methyl-2-((4-phenoxybutanoyl)glycyl)-2-azabicyclo[3.1.0]hexane-3-carbonyl)oxy)methyl)-1,3-dihydro-2H-pyrrolo[3,4-c]pyridine-2-carboxylate (4)

To a mixture of tert-butyl 6-(chloromethyl)-1,3-dihydro-2H-pyrrolo[3,4-c]pyridine-2-carboxylate (45 mg, 0.17 mmol) and (1S,3S,5S)-5-methyl-2-((4-phenoxybutanoyl) glycyl)-2-azabicyclo[3.1.0]hexane-3-carboxylic acid (50 mg, 0.14 mmol) in DMF (3 mL) was added K₂CO₃ (38 mg, 0.28 mmol) and the reaction mixture was stirred at 60° C. for 3 hours. The mixture was diluted with EtOAc, washed with water and brine, dried over anhydrous Na₂SO₄, filtered, and concentrated under reduced pressure to dryness. The residue was purified by prep-TLC (PE:EtOAc=1:1) to give tert-butyl 6-((((1S,3S,5S)-5-methyl-2-((4-phenoxybutanoyl)glycyl)-2-azabicyclo [3.1.0]hexane-3-carbonyl)oxy) methyl)-1,3-dihydro-2H-pyrrolo[3,4-c]pyridine-2-carboxylate (40 mug, yield 82.2%) as a white solid. LC/MS (ESI) m/z: 593 (M+H)⁺.

### Step 4: (2,3-dihydro-1H-pyrrolo[3,4-c]pyridin-6-yl)methyl (1S,3S,5S)-5-methyl-2-((4-phenoxybutanoyl)glycyl)-2-azabicyclo[3.1.0]hexane-3-carboxylate (Compound 66)

A solution of tert-butyl 6-((((1S,3S,5S)-5-methyl-2-((4-phenoxybutanoyl)glycyl)-2-azabicyclo[3.1.0]hexane-3-carbonyl)oxy)methyl)-1,3-dihydro-2H-pyrrolo[3,4-c]pyridine-2-carboxylate (40 mg, 0.07 mmol) in DCM (1 mL) and TFA (1 mL) was stirred under N₂ atmosphere at room temperature for 2 hours. The reaction mixture was concentrated under reduced pressure to dryness and the residue was purified by prep-HPLC to give Compound 66 (7.8 mg, yield 23.5%) as a yellow solid. ¹H NMR (400 MHz, CD₃OD): δ 8.58 (d, J=12.2 Hz, 1H), 7.55 (s, 1H), 7.26-7.19 (m, 2H), 6.91-6.85 (m, 3H), 5.32-5.18 (m, 2H), 4.91 (d, J=3.6 Hz, 1H), 4.68 (s, 4H), 4.30 (d, J=16.9 Hz, 1H), 4.12 (d, J=16.9 Hz, 1H), 4.01 (t, J=6.3 Hz, 2H), 3.37-3.33 (m, 1H), 2.49 (dd, J=16.8, 9.7 Hz, 3H), 2.16 (m, J=20.9, 14.0, 5.3 Hz, 3H), 1.28 (d, J=15.4 Hz, 3H), 1.16 (dd, J=5.7, 2.4 Hz, 1H), 0.82 (t, J=5.7 Hz, 1H). LC/MS (ESI) m/z: 493 (M+H)⁺. RT (Method A): 1.07 min.

### Scheme 27. Synthesis of (S)-N-((R)-1-(1H-pyrrolo[3,2-c]pyridin-2-yl)ethyl)-7-((2′,4′-difluoro-[1,1′-biphenyl]-4-carbonyl)glycyl)-1,4-dioxa-7-azaspiro[4.4]nonane-8-carboxamide (Compound 68)

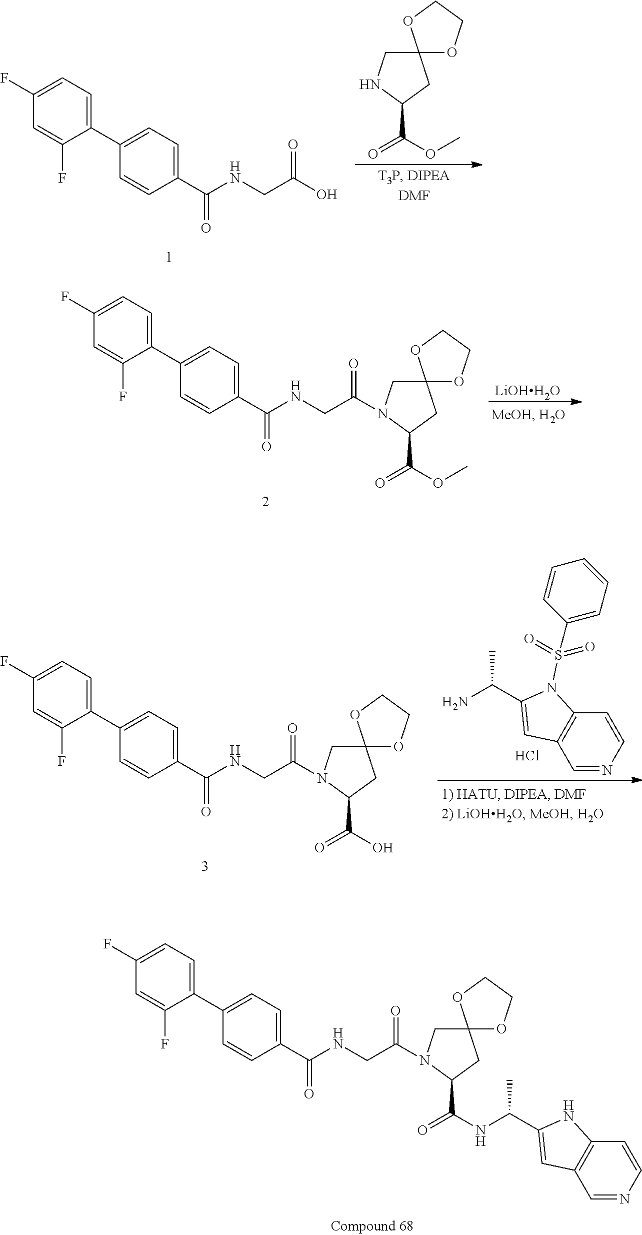

### Step 1: Methyl (S)-7-((2,4′-difluoro-[,1′-biphenyl]-4-carbonyl)glycyl)-1,4-dioxa-7-azaspiro[4.4]-nonane-8-carboxylate (2)

To a mixture of (2′,4′-difluoro-[1,1′-biphenyl]-4-carbonyl)glycine (40 mg, 0.15 mmol) and methyl (S)-1,4-dioxa-7-azaspiro[4.4]nonane-8-carboxylate (38.5 mug, 0.21 mmol) in DMF (3 mL) was added DIPEA (106.4 mg, 0.9 mmol) and T₃P (131.1 mg, 0.21 mmol, 50% wt. in EtOAc) under N₂ atmosphere at 0° C. and the mixture was stirred at room temperature for 2 hours. The mixture was quenched with saturated aq. NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel. 0-80% EtOAc in PE) to give methyl (S)-7-((2′,4′-difluoro-[1,1′-biphenyl]-4-carbonyl)glycyl)-1,4-dioxa-7-azaspiro[4.4]nonane-8-carboxylate (50 mg, yield 80.3%) as a colorless oil. LC/MS (ESI) (m/z): 461 (M+H)⁺.

### Step 2: (S)-7-((2,4′-difluoro-[1,1′-biphenyl]-4-carbonyl)glycyl)-1,4-dioxa-7-azaspiro [4.4]nonane-8-carboxylic acid (3)

To a solution of methyl (S)-7-((2′,4′-difluoro-[1,1′-biphenyl]-4-carbonyl)glycyl)-1,4-dioxa-7-azaspiro[4.4]nonane-8-carboxylate (50 mg, 0.11 mmol) in MeOH (3 mL) and water (1 mL) was added LiOH·H₂O (13.6 mg, 0.33 mmol) and the mixture was stirred at room temperature for 2 hours. The mixture was acidified with IN aq. HCl to pH˜3 and extracted with EtOAc twice. The combined organic layers were washed with brine, dried over anhydrous Na₂SO₄, filtered, and concentrated under reduced pressure to dryness to give (S)-7-((2′,4′-difluoro-[1,1′-biphenyl]-4-carbonyl)glycyl)-1,4-dioxa-7-azaspiro[4.4]nonane-8-carboxylic acid (40 mg, yield 82.3%) as a white solid, which was used directly in the next step without further purification. LC/MS (ESI) m/z: 447 (M+H)⁺.

### Step 3: (S)-N-((R)-1-(1H-pyrrolo[3,2-c]pyridin-2-yl)ethyl)-7-((2,4′-difluoro-[1,1′-biphenyl]-4-carbonyl)glycyl)-1,4-dioxa-7-azaspiro[4.4]nonane-8-carboxamide (Compound 68)

To a mixture of (S)-7-((2′,4′-difluoro-[1,1′-biphenyl]-4-carbonyl)glycyl)-1,4-dioxa-7-azaspiro[4.4]nonane-8-carboxylic acid (40 mg, 0.09 mmol) and (R)-1-(1H-pyrrolo[3,2-c]pyridin-2-yl)ethan-1-amine hydrochloride (28.8 mg, 0.18 mmol) in DMF (2 mL) was added DIPEA (69.4 mg, 0.54 mmol) and IIATU (51 mg, 0.14 mmol) under N₂ atmosphere and the reaction mixture was stirred at room temperature for 2 hours. To the mixture was added LiOH (4.8 mg, 0.20 mmol), MeOH (0.5 mL) and water (0.1 mL) and the mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=10:1) and further purified by prep-HPLC to give Compound 68 (20 mg, yield 37.8%) as a white solid. ¹H NMR (400 MHz, CD₃OD): δ 8.97 (d, J=21.0 Hz, 1H), 8.01 (dd, J=19.1, 7.3 Hz, 3H), 7.66 (d, J=8.1 Hz, 2H), 7.61-7.50 (m, 2H), 7.11 (dd, J=13.4, 6.0 Hz, 2H), 6.92 (s, 1H), 5.33 (q, J=7.0 Hz, 1H), 4.57 (t, J=8.0 Hz, 1H), 4.24 (dd, J=39.6, 16.7 Hz, 2H), 4.07-3.98 (m, 4H), 3.82 (q, J=10.7 Hz, 2H), 2.43 (dd, J=12.9, 8.3 Hz, 1H), 2.27 (dd, J=12.9, 7.9 Hz, 1H), 1.63 (d, J=7.0 Hz, 3H). LC/MS (ESI) m/z: 590 (M+H)⁺. RT (Method A): 1.25 min.

The following compounds were prepared based on Scheme 27:

| Cmpd # | Reactant A | Reactant B | Reactant C | Characterization Data |
|---|---|---|---|---|
| 70 | 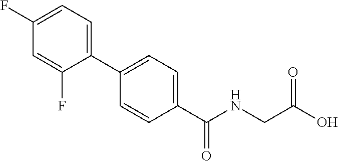 [SMILES: O=C(O)CNC(=O)c1ccc(-c2ccc(F)cc2F)cc1] | 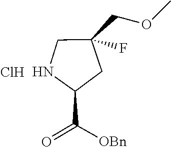 | 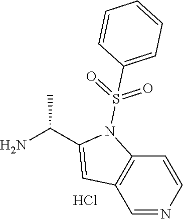 | ¹H NMR (400 MHz, CD₃OD): δ 8.90 (s, 1H), 8.02-7.89 (m, 3H), 7.67 (d, J = 8.1 Hz, 2H), 7.62-7.56 (m, 1H), 7.46 (d, J = 6.4 Hz, 1H), 7.11 (t, J = 9.7 Hz, 2H), 6.88 (s, 1H), 5.34 (q, J = 6.9 Hz, 1H), 4.67-4.59 (m, 1H), 4.38 (d, J = 17.0 Hz, 1H), 4.19 (d, J = 16.9 Hz, 1H), 4.12-3.89 (m, 2H), 3.79-3.66 (m, 2H), 3.45 (s, 3H), 2.54 (td, J = 15.3, 7.7 Hz, 1H), 2.36-2.16 (m, 1H), 1.64 (d, J = 7.0 Hz, 3H). LC/MS (ESI) m/z: 594 (M + H)⁺. RT (Method A): 1.29 min. |
| 85 | 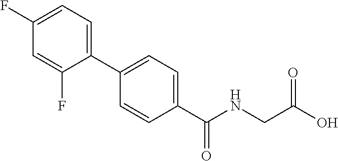 [SMILES: O=C(O)CNC(=O)c1ccc(-c2ccc(F)cc2F)cc1] | 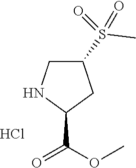 | 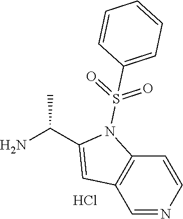 | ¹H NMR (400 MHz, CD₃OD): δ 8.96 (d, J = 18.9 Hz, 1H), 8.05 (d, J = 6.5 Hz, 1H), 7.99 (d, J = 8.1 Hz, 2H), 7.66 (d, J = 8.0 Hz, 2H), 7.58 (dd, J = 11.4, 7.9 Hz, 2H), 7.10 (d, J = 8.4 Hz, 2H), 6.95 (d, J = 34.3 Hz, 1H), 5.32 (q, J = 7.2 Hz, 1H), 4.75-4.70 (m, 1H), 4.37-4.12 (m, 5H), 3.11 (s, 3H), 2.89-2.73 (m, 1H), 2.58-2.41 (m, 1H), 1.65 (d, J = 7.0 Hz, 3H). LC/MS (ESI) m/z: 610 (M + H)⁺. RT (Method A): 1.17 min. |
| 132^{c} | 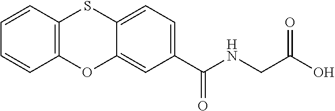 [SMILES: O=C(O)CNC(=O)c1ccc2c(c1)Oc1ccccc1S2] | 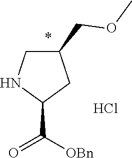 | 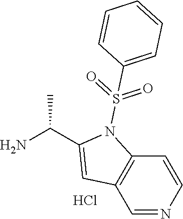 | ¹H NMR (400 MHz, CD₃OD): δ 8.64 (s, 1H), 7.89 (d, J = 5.9 Hz, 1H), 7.60 (d, J = 13.6 Hz, 2H), 7.28 (t, J = 8.9 Hz, 1H), 7.21- 7.15 (m, 2H), 7.09 (d, J = 6.4 Hz, 2H), 7.03 (d, J = 8.1 Hz, 1H), 6.51 (s, 1H), 5.31 (d, J = 6.5 Hz, 1H), 4.40 (dd, J = 17.9, 12.1 Hz, 2H), 4.10 (d, J = 16.9 Hz, 1H), 3.97-3.90 (m, 1H), 3.50-3.42 (m, 3H), 3.34 (s, 3H), 2.66 (s, 1H), 2.39 (d, J = 12.0 Hz, 1H), 1.85-1.75 (m, 1H), 1.60 (d, J = 6.9 Hz, 3H). LC/MS (ESI) m/z: 586 (M + H)⁺. RT (Method A): 1.66 min. |
| 133^{c} | 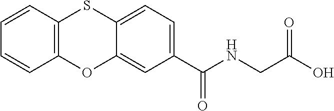 [SMILES: O=C(O)CNC(=O)c1ccc2c(c1)Oc1ccccc1S2] | 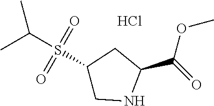 | 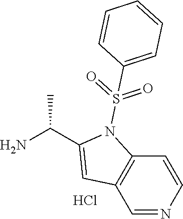 | ¹H NMR (400 MHz, CD₃OD): δ 8.66 (s, 1H), 7.94 (d, J = 5.8 Hz, 1H), 7.60-7.48 (m, 2H), 7.27-7.15 (m, 4H), 7.10-7.02 (m, 2H), 6.54 (s, 1H), 5.33-5.26 (m, 1H), 4.73-4.64 (m, 1H), 4.29 (s, 2H), 4.15 (d, J = 6.5 Hz, 2H), 3.48-3.38 (m, 1H), 3.13 (s, 1H), 2.86-2.70 (m, 1H), 2.52-2.39 (m, 1H), 1.61 (d, J = 6.9 Hz, 3H), 1.39 (d, J = 6.8 Hz, 6H). LC/MS (ESI) m/z: 648 (M + H)⁺. RT (Method A): 1.66 min. |
| 145^{c} | 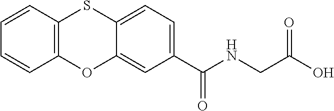 [SMILES: O=C(O)CNC(=O)c1ccc2c(c1)Oc1ccccc1S2] | 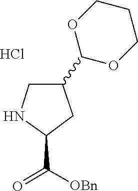 [SMILES: Cl ; O=C(OCc1ccccc1)[C@@H]1CC(C2OCCCO2)CN1] | 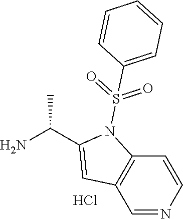 | ¹H NMR (400 MHz, CD₃OD): δ 8.68 (s, 1H), 7.96 (d, J = 4.8 Hz, 1H), 7.62-7.51 (m, 2H), 7.29-7.14 (m, 4H), 7.10-7.01 (m, 2H), 6.54 (s, 1H), 5.28 (q, J = 7.1 Hz, 1H), 4.61 (d, J = 4.6 Hz, 1H), 4.55-4.48 (m, 1H), 4.28 (d, J = 16.5 Hz, 1H), 4.16 (d, J = 16.7 Hz, 1H), 4.09 (d, J = 11.7 Hz, 2H), 3.82 (t, J = 10.1 Hz, 3H), 3.70-3.64 (m, 1H), 2.75-2.54 (m, 1H), 2.25 (dd, J = 15.6, 5.4 Hz, 1H), 2.11-2.00 (m, 2H), 1.59 (d, J = 6.7 Hz, 3H), 1.37 (d, J = 12.5 Hz, 1H). LC/MS (ESI) m/z: 628 (M + H)⁺. RT (Method A): 1.85 min. |
| 148^{c} | 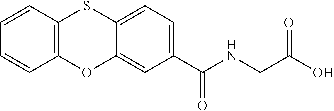 [SMILES: O=C(O)CNC(=O)c1ccc2c(c1)Oc1ccccc1S2] | 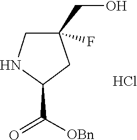 [SMILES: O=C(OCc1ccccc1)[C@@H]1C[C@](F)(CO)CN1 ; Cl] | 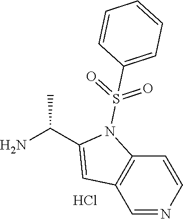 | ¹H NMR (400 MHz, CD₃OD): δ 8.64 (s, 1H), 7.87 (d, J = 5.6 Hz, 1H), 7.60 (d, J = 13.2 Hz, 2H), 7.28 (d, J = 8.0 Hz, 1H), 7.19 (dd, J = 15.6, 7.6 Hz, 2H), 7.11-7.01 (m, 3H), 6.52 (s, 1H), 5.31 (q, J = 6.6 Hz, 1H), 4.60 (dd, J = 19.1, 10.0 Hz, 2H), 4.37 (d, J = 16.7 Hz, 1H), 4.13 (d, J = 16.8 Hz, 1H), 4.00 (dd, J = 13.6, 6.7 Hz, 1H), 3.86 (d, J = 3.8 Hz, 1H), 3.82 (d, J = 6.4 Hz, 1H), 2.50 (td, J = 14.7, 7.2 Hz, 1H), 2.32-2.16 (m, 1H), 1.61 (d, J = 6.8 Hz, 3H). LC/MS (ESI) m/z: 590 (M + H)⁺. RT (Method A): 3.84 min. |
| 150^{c} | 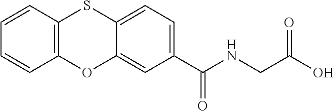 [SMILES: O=C(O)CNC(=O)c1ccc2c(c1)Oc1ccccc1S2] | 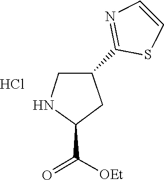 [SMILES: Cl ; CCOC(=O)[C@@H]1C[C@@H](c2nccs2)CN1] | 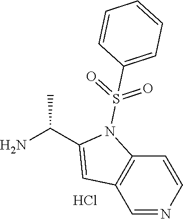 | ¹H NMR (400 MHz, CD₃OD): δ 8.66 (d, J = 4.0 Hz, 1H), 7.93 (dd, J = 19.8, 5.8 Hz, 1H), 7.75 (s, 1H), 7.61-7.50 (m, 3H), 7.27- 7.17 (m, 3H), 7.16-7.12 (m, 1H), 7.09 (d, J = 8.7 Hz, 1H), 7.03 (d, J = 8.1 Hz, 1H), 6.54 (d, J = 8.2 Hz, 1H), 5.31 (dd, J = 14.0, 7.0 Hz, 1H), 4.57 (s, 2H), 4.40 (dd, J = 30.3, 12.5 Hz, 1H), 4.29-4.17 (m, 2H), 3.99 (dd, J = 17.5, 9.7 Hz, 1H), 2.79-2.33 (m, 2H), 1.61 (d, J = 6.8 Hz, 3H). LC/MS (ESI) m/z: 625 (M + H)⁺. RT (Method A): 1.84 min. |
| 164^{c} | 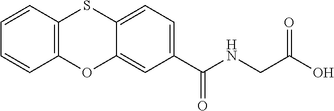 [SMILES: O=C(O)CNC(=O)c1ccc2c(c1)Oc1ccccc1S2] | 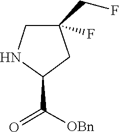 [SMILES: O=C(OCc1ccccc1)[C@@H]1C[C@](F)(CF)CN1] | 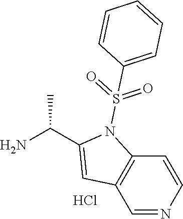 | ¹H NMR (400 MHz, CD₃OD): δ 8.79 (s, 1H), 8.50 (s, 1H), 7.99- 7.94 (m, 1H), 7.58 (s, 1H), 7.55 (s, 1H), 7.33 (s, 1H), 7.28 (d, J = 8.4 Hz, 1H), 7.21-7.16 (m, 2H), 7.09 (s, 1H), 7.03 (d, J = 8.3 Hz, 1H), 6.74 (s, 1H), 5.35-5.31 (m, 1H), 4.71 (s, 2H), 4.66 (s, 1H), 4.33 (s, 1H), 4.16 (d, J = 17.1 Hz, 3H), 2.63-2.56 (m, 1H), 2.30 (ddd, J = 11.4, 6.6, 2.1 Hz, 1H), 1.63 (d, J = 6.5 Hz, 3H). LC/MS (ESI) (m/z): 592 (M + H)⁺. RT (Method A): 1.84 min. |
| 191^{c} | 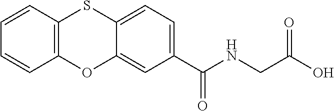 [SMILES: O=C(O)CNC(=O)c1ccc2c(c1)Oc1ccccc1S2] | 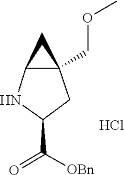 | 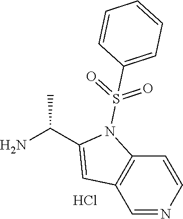 | ¹H NMR (400 MHz, CD₃OD): δ 8.65 (d, J = 0.8 Hz, 1H), 7.92 (d, J = 5.8 Hz, 1H), 7.57 (dd, J = 7.0, 1.7 Hz, 2H), 7.25-7.22 (m, 1H), 7.21-7.15 (m, 2H), 7.13 (d, J = 5.8 Hz, 1H), 7.08 (dd, J = 11.3, 3.7 Hz, 1H), 7.03 (dd, J = 8.0, 1.1 Hz, 1H), 6.51 (s, 1H), 5.28-5.22 (m, 1H), 4.82 (d, J = 4.4 Hz, 1H), 4.51 (d, J = 16.7 Hz, 1H), 4.26 (d, J = 16.7 Hz, 1H), 3.57-3.51 (m, 2H), 3.38 (d, J = 4.7 Hz, 4H), 2.67 (t, J = 11.9 Hz, 1H), 2.11 (dd, J = 13.3, 4.3 Hz, 1H), 1.59 (d, J = 7.0 Hz, 3H), 1.43 (dd, J = 5.6, 2.7 Hz, 1H), 1.03 (t, J = 5.4 Hz, 1H). LC/MS (ESI) m/z: 598 (M + H)⁺. RT (Method A): 1.59 min. |
| 289^{c} | 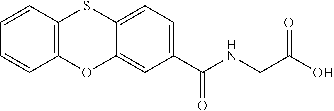 [SMILES: O=C(O)CNC(=O)c1ccc2c(c1)Oc1ccccc1S2] | 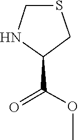 | 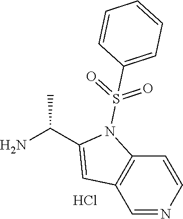 | ¹H NMR (400 MHz, CD₃OD): δ 8.99 (s, 1H), 8.70 (s, 1H), 8.17- 8.04 (m, 2H), 7.72 (d, J = 6.7 Hz, 1H), 7.60-7.55 (m, 2H), 7.50 (d, J = 1.7 Hz, 1H), 7.35 (dd, J = 8.1, 1.8 Hz, 1H), 7.25 (d, J = 8.1 Hz, 1H), 7.24-7.20 (m, 2H), 7.19-7.17 (m, 1H), 7.14 (dd, J = 10.6, 1.6 Hz, 3H), 7.10 (s, 1H), 7.08 (s, 1H), 7.06-7.03 (m, 1H), 7.00 (d, J = 1.0 Hz, 1H), 6.96 (s, 1H), 6.78 (s, 1H), 5.40 (dd, J = 9.5, 2.9 Hz, 1H), 5.35-5.30 (m, 1H), 5.05 (d, J = 8.7 Hz, 1H), 4.98 (dd, J = 6.9, 3.2 Hz, 1H), 4.89 (s, 1H), 4.81 (s, 1H), 4.79 (s, 1H), 4.42 (d, J = 15.8 Hz, 1H), 4.30 (s, 2H), 3.98- 3.93 (m, 1H), 3.48-3.40 (m, 3H), 3.26-3.22 (m, 2H), 1.74 (d, J = 7.0 Hz, 3H), 1.65 (d, J = 7.1 Hz, 3H). LC/MS (ESI) m/z: 560 (M + H)⁺. RT (Method A): 1.45 min. |
| 302 | 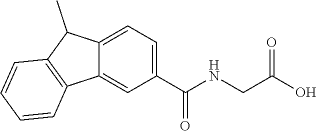 | 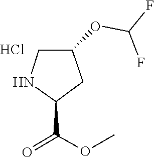 | 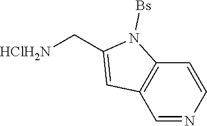 | ¹H-NMR (400 MHz, CD₃OD): δ 8.57 (d, J = 5.3 Hz, 1H), 8.29 (s, 1H), 7.82 (dd, J = 11.7, 5.4 Hz, 3H), 7.62 (d, J = 8.0 Hz, 1H), 7.57 (d, J = 5.8 Hz, 1H), 7.37 (t, J = 3.5 Hz, 2H), 7.17-7.12 (m, 1H), 6.73-6.51 (m, 1H), 6.49 (s, 1H), 5.34 (t, J = 4.6 Hz, 1H), 5.06 (s, 1H), 4.69 (s, 1H), 4.61 (s, 1H), 4.57 (s, 1H), 4.30 (s, 1H), 4.27 (s, 1H), 4.02 (s, 1H), 3.92 (d, J = 11.1 Hz, 1H), 2.53-2.47 (m, 1H), 2.32-2.26 (m, 1H), 1.54 (d, J = 7.4 Hz, 3H). LC/MS (ESI) m/z: 574 (M + H)⁺. RT (Method A): 1.46 min. |
| 320^{a} | 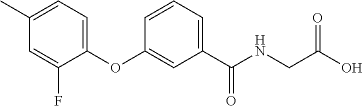 | 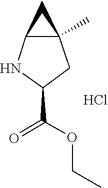 | 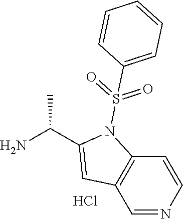 | ¹H NMR (400 MHz, CD₃OD): δ 8.65 (s, 1H), 7.95 (d, J = 5.8 Hz, 1H), 7.60 (d, J = 7.7 Hz, 1H), 7.47-7.42 (m, 2H), 7.15 (dd, J = 8.6, 3.9 Hz, 2H), 7.04-6.93 (m, 3H), 6.49 (s, 1H), 5.23 (q, J = 6.6 Hz, 1H), 4.81 (d, J = 7.4 Hz, 1H), 4.46 (d, J = 16.6 Hz, 1H), 4.25 (d, J = 16.6 Hz, 1H), 3.36 (dd, J = 5.8, 2.3 Hz, 1H), 2.41 (t, J = 12.3 Hz, 1H), 2.31 (s, 3H), 2.16 (dd, J = 13.4, 3.9 Hz, 1H), 1.55 (d, J = 6.9 Hz, 3H), 1.30 (s, 3H), 1.28-1.26 (m, 1H), 0.80 (t, J = 5.5 Hz, 1H). LC/MS (ESI) m/z: 570 (M + H)⁺. RT (Method A): 1.60 min. |
| 338^{b} | 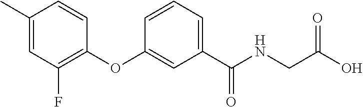 | 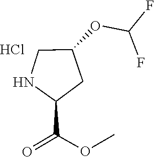 | 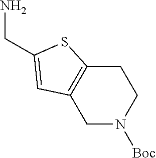 [SMILES: CC(C)(C)OC(=O)N1CCc2sc(CN)cc2C1] | ¹H NMR (400 MHz, CD₃OD): δ 7.58-7.53 (m, 1H), 7.42 (t, J = 7.9 Hz, 2H), 7.11-7.06 (m, 2H), 7.02 (t, J = 8.3 Hz, 2H), 6.69- 6.30 (m, 2H), 4.99-4.96 (m, 1H), 4.69-4.62 (m, 1H), 4.58 (s, 1H), 4.55-4.51 (m, 1H), 4.50-4.39 (m, 2H), 4.25-4.10 (m, 2H), 3.97-3.90 (m, 1H), 3.81-3.76 (m, 2H), 3.09-3.06 (m, 1H), 2.80-2.73 (m, 2H), 2.46-2.38 (m, 1H), 2.36 (s, 3H), 2.21- 2.17 (m, 1H), 2.06-2.01 (m, 1H). LCMS (ESI) m/z: 617 (M + H)⁺. RT (Method A): 1.61 min. |

^{a}HATU was used in place of T₃P in Step 1.
^{b}HCl/1,4-dioxane was used for deprotection in Step 4.
^{c}The final coupling product was isolated prior to the final deprotection step.

### Scheme 28. Synthesis of (1S,3S,5S)-N-((S)-5-(aminomethyl)-2,3-dihydro-1H-pyrrolizin-1-yl)-5-methyl-2-((4-phenoxybutanoyl)glycyl)-2-azabicyclo[3.1.0]hexane-3-carboxamide (Compound 69)

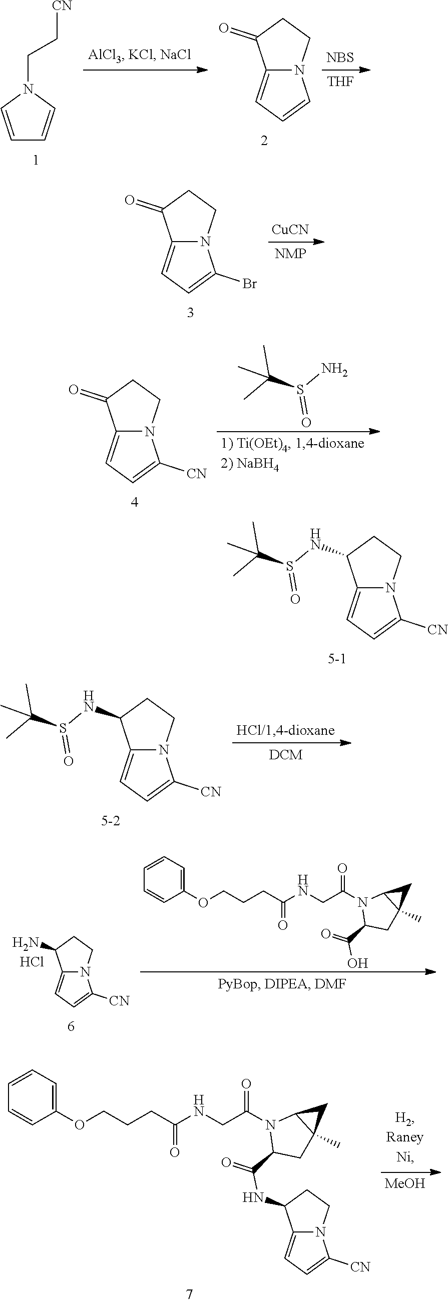

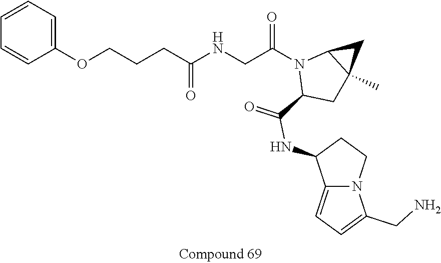 [SMILES: C[C@@]12C[C@@H]1N(C(=O)CNC(=O)CCCOc1ccccc1)[C@H](C(=O)N[C@H]1CCn3c(CN)ccc31)C2]

### Step 1: 2,3-Dihydro-JH-pyrrolizin-J-one (2)

A mixture of AlCl₃ (34.67 g, 260.70 mmol), KCl (7.02 g, 94.14 mmol) and NaCl (6.77 g, 115.90 mmol) was heated to form a clear straw-brown liquid (about 160° C.), when the temperature fell to 120° C., 3-(1H-pyrrol-1-yl)propanenitrile (8.70 g, 72.41 mmol) was added and the mixture was stirred at 120° C. for 3 minutes. The reaction mixture was poured into ice-water (100 mL) and 2M aq. KOH solution (20 mL) was added. The mixture was stirred at 80° C. for 30 minutes. The mixture was diluted with water and extracted with DCM twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give 2,3-dihydro-H-pyrrolizin-1-one (6.76 g, yield 77.1%) as a white solid. ¹H NMR (400 MHz, CD₃OD) δ 7.21 (d, J=1.4 Hz, 1H), 6.68 (dd, J=4.0, 0.9 Hz, 1H), 6.52 (dd, J=4.0, 2.2 Hz, 1H), 4.35-4.31 (m, 2H), 3.10-3.05 (m, 2H). LC/MS (ESI) (m/z): 122 (M+H)⁺.

### Step 2: 5-Bromo-2,3-dihydro-1H-pyrrolizin-1-one (3)

To a solution of 2,3-dihydro-1H-pyrrolizin-1-one (6.50 g, 53.66 mmol) in THF (70 mL) was added NBS (12.43 g, 69.84 mmol) at −50° C. and the reaction mixture was stirred at −50° C. for 4 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-37% EtOAc in PE) to give 5-bromo-2,3-dihydro-1H-pyrrolizin-1-one (8.50 g, yield 79.2%) as red solid. ¹H NMR (400 MHz, CD₃OD) δ 6.70 (d, J=4.1 Hz, 1H), 6.52 (d, J=4.1 Hz, 1H), 4.24-4.20 (m, 2H), 3.14-3.09 (m, 2H). LC/MS (ESI) m/z: 200 (M+H)⁺.

### Step 3: I-Oxo-2,3-dihydro-1H-pyrrolizine-5-carbonitrile (4)

To a solution of 5-bromo-2,3-dihydro-1H-pyrrolizin-1-one (4.50 g, 22.50 mmol) in NMP (70 mL) was added CuCN (6.04 g, 67.49 mmol) under N₂ atmosphere. The mixture was stirred under N₂ atmosphere at 180° C. for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-38% EtOAc in PE) to give 1-oxo-2,3-dihydro-1H-pyrrolizine-5-carbonitrile (330 mg, yield 10.0%) as a yellow solid. LC/MS (ESI) m/z: 147 (M+H)⁺.

### Step 4: (S)-N-((R)-5-cyano-2,3-dihydro-1H-pyrrolizin-1-yl)-2-methylpropane-2-sulfinamide and (S)-N-((S)-5-cyano-2,3-dihydro-1H-pyrrolizin-1-yl)-2-methylpropane-2-sulfinamide (5)

To a mixture of 1-oxo-2,3-dihydro-1H-pyrrolizine-5-carbonitrile (200 mg, 1.37 mmol) and (S)-2-methylpropane-2-sulfinamide (415 mg, 3.42 mmol) in 1,4-dioxane (5 mL) was added Ti(OEt)₄ (1.25 g, 5.48 mmol) under N₂ atmosphere at 0° C. The mixture was degassed under N₂ atmosphere for three times and stirred at 90° C. for 2 hours. The reaction mixture was cooled to room temperature. To the reaction mixture, NaBH₄ (207 mg, 5.47 mmol) was added in portions and the resulting mixture was stirred at room temperature for 2 hours. The mixture was quenched with ice-water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give (S)-N-((S)-5-cyano-2,3-dihydro-1H-pyrrolizin-1-yl)-2-methylpropane-2-sulfinamide (167 mg, yield 48.5%) and (S)-N-((R)-5-cyano-2,3-dihydro-1H-pyrrolizin-1-yl)-2-methylpropane-2-sulfinamide (150 mg, yield 43.6%) as a yellow solid. LC/MS (ESI) m/z: 252 (M+H)⁺.

### Step 5: (S)-1-amino-2,3-dihydro-1H-pyrrolizine-5-carbonitrile hydrochloride(6)

To a solution of (S)-N-((S)-5-cyano-2,3-dihydro-1H-pyrrolizin-1-yl)-2-methylpropane-2-sulfinamide (60 mg, 0.24 mmol) in DCM (1.2 mL) was added HCl/1,4-dioxane (1.4 mL, 4M) and the mixture was stirred under N₂ atmosphere at room temperature for 30 minutes. The reaction mixture was concentrated under reduced pressure to dryness to give (S)-1-amino-2,3-dihydro-1H-pyrrolizine-5-carbonitrile hydrochloride (43 mg, yield 98.4%) as a yellow solid, which was used directly in the next step without further purification. LC/MS (ESI) m/z: 148 (M+H)⁺.

### Step 6: (1S,3S,5S)-N-((S)-5-cyano-2,3-dihydro-1H-pyrrolizin-1-yl)-5-methyl-2-((4-phenoxybutanoyl)glycyl)-2-azabicyclo[3.1.0]hexane-3-carboxamide (7)

To a mixture of (S)-1-amino-2,3-dihydro-1H-pyrrolizine-5-carbonitrile hydrochloride (43 mg, 0.23 mmol) and (1 S,3S,5S)-5-methyl-2-((4-phenoxybutanoyl)glycyl)-2-azabicyclo[3.1.0]hexane-3-carboxylic acid (90 mg, 0.25 mmol) in DMF (3 mL) was added DIPEA (161 mg, 0.37 mmol) and T₃P (238 mg, 0.15 mmol, 50% wt. in EtOAc) under N₂ atmosphere and the mixture was stirred at room temperature for 1 hour. The mixture was diluted with EtOAc, washed with saturated aq. NaIICO₃ solution and brine, dried over anhydrous Na₂SO₄, filtered, and concentrated under reduced pressure to dryness. The residue was purified by flash chromatography (silica gel, 0-3% MeOH in DCM) to give (1S,3S,5S)-N-((S)-5-cyano-2,3-dihydro-1H-pyrrolizin-1-yl)-5-methyl-2-((4-phenoxybutanoyl)glycyl)-2-azabicyclo[3.1.0]hexane-3-carboxamide (74 mg, yield 63.6%) as a light-yellow oil. LC/MS (ESI) m/z: 490 (M+H)⁺.

### Step 7: (1S,3S,5S)-N-((S)-5-(aminomethyl)-2,3-dihydro-1H-pyrrolizin-1-yl)-5-methyl-2-((4-phenoxybutanoyl)glycyl)-2-azabicyclo[3.1.0]hexane-3-carboxamide (Compound 69)

To a solution of(1S,3S,5S)-N-((S)-5-cyano-2,3-dihydro-1H-pyrrolizin-1-yl)-5-methyl-2-((4-phenoxybutanoyl)glycyl)-2-azabicyclo[3.1.0]hexane-3-carboxamide (74 mg, 0.15 mmol) in MeOH (5 mL) was added Raney Ni (20 mg, wet.), the mixture was degassed under N₂ atmosphere for three times and stirred under a H₂ balloon at room temperature for 2 hours. The mixture was filtered, and the filtrate was concentrated to dryness. The residue was purified by prep-HPLC to give Compound 69 (10.0 mg, yield 13.4%) as a white solid. ¹H NMR (400 MHz, CD₃OD): δ 7.28-7.22 (m, 2H), 6.92-6.88 (m, 3H), 6.29 (d, J=3.6 Hz, 1H), 5.91 (t, J=4.8 Hz, 1H), 5.22-5.12 (m, 1H), 4.74-4.66 (m, 1H), 4.25 (d, J=16.7 Hz, 1H), 4.11-4.06 (m, 3H), 4.03-3.88 (m, 4H), 3.29-3.25 (m, 1H), 2.91-2.80 (m, 1H), 2.47-2.30 (m, 4H), 2.13-2.03 (m, 3H), 1.29-1.24 (m, 4H), 0.80 (t, J=5.6 Hz, 1H). LC/MS (ESI) m/z: 494 (M+H)⁺. RT (Method A): 1.04 min.

### Scheme 29. (2S,4R)-N-((R)-1-(1H-pyrrolo[3,2-c]pyridin-2-yl)ethyl)-4-fluoro-4-(fluoromethyl)-1-((4-(4-fluorophenoxy)benzoyl)glycyl)pyrrolidine-2-carboxamide (Compound 89)

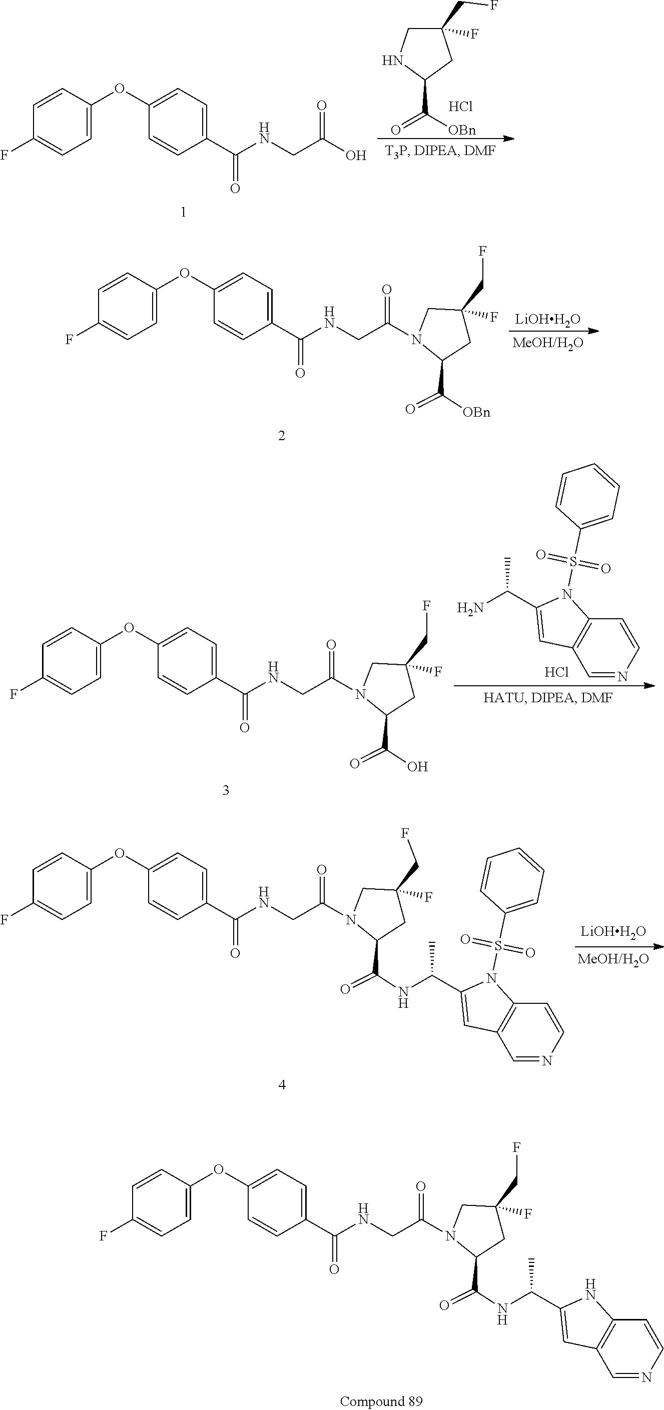

### Step 1: Benzyl (2S,4R)-4-fluoro-4-(fluoromethyl)-1-((4-(4-fluorophenoxy)benzoyl) glycyl)pyrrolidine-2-carboxylate (2)

To a mixture of (4-(4-fluorophenoxy)benzoyl)glycine (100 mg, 0.34 mol) and benzyl (2S,4R)-4-fluoro-4-(fluoromethyl)pyrrolidine-2-carboxylate hydrochloride (88 mg, 0.34 mmol) in DMF (3 mL) was added DIPEA (223 mg, 1.73 mmol) and T₃P (197 mg, 0.52 mmol, 50% wt. in EtOAc) under N₂ atmosphere at 0° C. and the mixture was stirred at room temperature for 2 hours. The mixture was quenched with saturated aq. NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (118 mg, yield 64.8%) as a colorless oil. LC/MS (ESI) (m/z): 527 (M+H)⁺.

### Step 2: (2S,4R)-4-fluoro-4-(fluoromethyl)-1-((4-(4-fluorophenoxy)benzoyl)glycyl) pyrrolidine-2-carboxylic acid (3)

To a solution of benzyl (2S,4R)-4-fluoro-4-(fluoromethyl)-1-((4-(4-fluorophenoxy) benzoyl)glycyl)pyrrolidine-2-carboxylate (118 mg, 0.22 mmol) in MeOH (2 mL) and water (0.5 mL) was added LiOH H₂O (18 mg, 0.01 mmol) and the mixture was stirred at room temperature for 2 hours. The mixture was acidified with IN aq. HCl to pH˜3 and extracted with EtOAc twice. The combined organic layers were washed with brine, dried over anhydrous Na₂SO₄, filtered, and concentrated under reduced pressure to dryness to give the title compound (95 mg, yield 96.9%) as a yellow oil, which was used directly in the next step without further purification. LC/MS (ESI) m/z: 437 (M+H)⁺.

### Step 3: (2S,4R)-4-fluoro-4-(fluoromethyl)-1-((4-(4 fluorophenoxy)benzoyl)glycyl)-N-((R)-1-(1-(phenylsulfonyl)-H-pyrrolo[3,2-c]pyridin-2-yl)ethyl)pyrrolidine-2-carboxamide (4)

To a mixture of (2S,4R)-4-fluoro-4-(fluoromethyl)-1-((4-(4-fluorophenoxy)benzoyl) glycyl)pyrrolidine-2-carboxylic acid (30 mg, 0.069 mol) and (R)-1-(1-(phenylsulfonyl)-1H-pyrrolo[3,2-c]pyridin-2-yl)ethan-1-amine hydrochloride (31 mg, 0.10 mmol) in DMF (3 mL) was added DIPEA (45 mg, 0.35 mmol) and HATU (32 mg, 0.084 mol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=20:1) to give the title compound (10 mg, yield 20.4%) as a colorless oil. LC/MS (ESI) n/z: 720 (M+H).

### Step 4: (2S,4R)-N-((R)-1-(1H-pyrrolo[3,2-c]pyridin-2-yl)ethyl)-4-fluoro-4-(fluoromethyl)-1-((4-(4-fluorophenoxy)benzoyl)glycyl)pyrrolidine-2-carboxamide (Compound 89)

To a solution of (2S,4R)-4-fluoro-4-(fluoromethyl)-1-((4-(4-fluorophenoxy)benzoyl) glycyl)-N-((R)-1-(1-(phenylsulfonyl)-1H-pyrrolo[3,2-c]pyridin-2-yl)ethyl)pyrrolidine-2-carboxamide (10 mg, 0.014 mol) in MeOH (2 mL) and water (1 mL) was added LiOH·H₂O (2 mg, 0.048 mol), and the mixture was stirred at room temperature for 2 hours. The mixture was diluted with EtOAc, washed water and brine, dried over anhydrous Na₂SO₄, filtered, and concentrated under reduced pressure to dryness. The residue was purified by prep-IIPLC to give Compound 89 (1.1 mg, yield 13.7%) as a white solid. ¹H NMR (400 MHz, CD₃OD): δ 8.69-8.55 (m, 1H), 8.06-7.82 (m, 3H), 7.37-6.97 (m, 7H), 6.57 (d, J=33.1 Hz, 1H), 5.31 (q, J=6.5 Hz, 1H), 4.69 (dd, J=39.3, 18.8 Hz, 3H), 4.37 (d, J=17.0 Hz, 1H), 4.19-4.07 (m, 2H), 3.98 (dd, J=32.5, 12.4 Hz, 1H), 2.71-2.52 (m, 1H), 2.35-2.18 (m, 1H), 1.63 (dd, J=19.0, 6.5 Hz, 3H). LC/MS (ESI) m/z: 580 (M+(H)⁺. RT (Method A): 1.28 min.

The following compounds were prepared based on Scheme 29:

| Cmpd # | Reactant A | Reactant B | Reactant C | Characterization Data |
|---|---|---|---|---|
| 146^{a} | 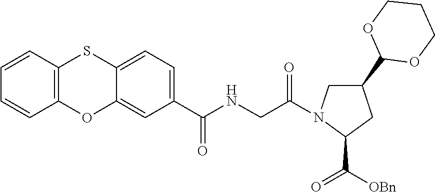 [SMILES: O=C(NCC(=O)N1C[C@@H](C2OCCCO2)C[C@H]1C(=O)OCc1ccccc1)c1ccc2c(c1)Oc1ccccc1S2] | | 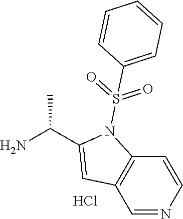 | ¹H NMR (400 MHz, DMSO-d6): δ 11.13 (s, 1H), 8.84 (s, 1H), 8.68 (s, 1H), 8.54 (d, J = 8.0 Hz, 1H), 7.88 (d, J = 5.4 Hz, 1H), 7.64 (s, 2H), 7.43-7.37 (m, 1H), 7.31 (d, J = 7.6 Hz, 1H), 7.28-7.24 (m, 1H), 7.13 (t, J = 8.0 Hz, 2H), 6.98 (d, J = 4.8 Hz, 1H), 6.41 (s, 1H), 5.19-5.09 (m, 1H), 4.57 (d, J = 5.8 Hz, 1H), 4.26 (dd, J = 18.5, 10.6 Hz, 2H), 3.98 (d, J = 11.0 Hz, 3H), 3.81 (t, J = 8.6 Hz, 1H), 3.72- 3.63 (m, 2H), 3.42 (t, J = 10.0 Hz, 1H), 2.23-2.14 (m, 1H), 2.00 (d, J = 9.7 Hz, 1H), 1.81 (dd, J = 20.2, 10.2 Hz, 2H), 1.47 (d, J = 6,6 Hz, 3H), 1.32 (d, J = 13.7 Hz, 1H). LC/MS (ESI) m/z: 628 (M + H)⁺. RT (Method A): 2.09 min. |
| 160 | 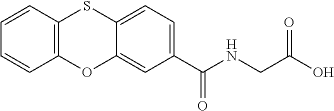 [SMILES: O=C(O)CNC(=O)c1ccc2c(c1)Oc1ccccc1S2] | 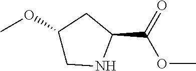 | 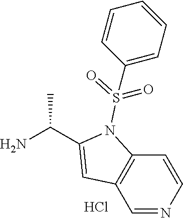 | ¹H NMR (400 MHz, CD₃OD): δ 8.64 (s, 1H), 7.88 (d, J = 5.8 Hz, 1H), 7.62-7.55 (m, 2H), 7.26 (d, J = 7.9 Hz, 1H), 7.21-7.15 (m, 2H), 7.09 (d, J = 5.9 Hz, 2H), 7.02 (d, J = 7.9 Hz, 1H), 6.51 (s, 1H), 5.33-5.28 (m, 1H), 4.47 (t, J = 8.0 Hz, 1H), 4.36 (d, J = 16.5 Hz, 1H), 4.21-4.13 (m, 2H), 3.82 (q, J = 11.6 Hz, 2H), 3.39 (s, 3H), 2.45-2.37 (m, 1H), 2.13-2.05 (m, 1H), 1.60 (d, J = 6.8 Hz, 3H). LC/MS (ESI) m/z: 572 (M + H)⁺. RT (Method A): 1.80 min. |
| 161 | 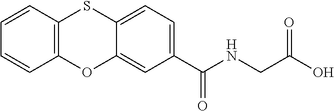 [SMILES: O=C(O)CNC(=O)c1ccc2c(c1)Oc1ccccc1S2] | 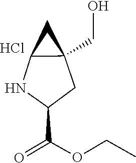 | 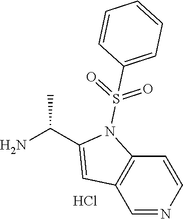 | ¹H NMR (400 MHz, CD₃OD): δ 8.65 (s, 1H), 7.92 (d, J = 5.0 Hz, 1H), 7.56 (s, 2H), 7.22 (dd, J = 19.3, 8.7 Hz, 3H), 7.15 (d, J = 7.5 Hz, 1H), 7.09 (d, J = 6.9 Hz, 1H), 7.03 (d, J = 8.1 Hz, 1H), 6.51 (s, 1H), 5.25 (d, J = 6.7 Hz, 1H), 4.51 (d, J = 16.6 Hz, 1H), 4.27 (d, J = 16.2 Hz, 1H), 3.69 (d, J = 11.8 Hz, 1H), 3.52 (d, J = 9.8 Hz, 2H), 2.71 (t, J = 12.0 Hz, 1H), 2.11 (dd, J = 37.3, 24.1 Hz, 2H), 1.59 (d, J = 6.8 Hz, 3H), 1.03 (t, J = 5.1 Hz, 1H), 0.91-0.87 (m, 1H). LC/MS (ESI) m/z: 584 (M + H)⁺. RT (Method A): 1.38 min. |
| 177^{b} | 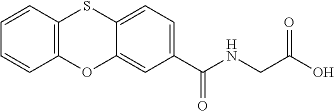 [SMILES: O=C(O)CNC(=O)c1ccc2c(c1)Oc1ccccc1S2] | 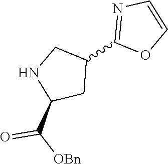 [SMILES: O=C(OCc1ccccc1)[C@@H]1CC(c2ncco2)CN1] | 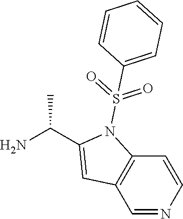 | ¹H NMR (400 MHz, CD₃OD): δ 8.66 (s, 1H), 7.92 (d, J = 20.7 Hz, 2H), 7.56 (s, 2H), 7.21 (d, J = 44.7 Hz, 5H), 7.07 (d, J = 14.2 Hz, 2H), 6.53 (s, 1H), 5.31 (s, 1H), 4.61 (s, 1H), 4.31-4.14 (m, 3H), 4.04 (s, 2H), 2.62 (s, 1H), 2.48 (s, 1H), 1.61 (d, J = 6.3 Hz, 3H). LC/MS (ESI) m/z: 609 (M + H)⁺. RT (Method A): 1.39 min. |
| 321^{b} | 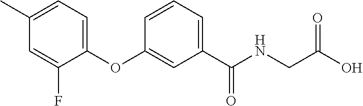 | 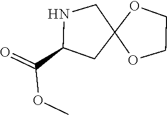 | 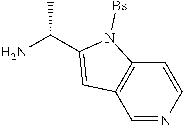 | ¹H NMR (400 MHz, CD₃OD): δ 8.66 (s, 1H), 7.95 (d, J = 5.9 Hz, 1H), 7.60 (d, J = 7.9 Hz, 1H), 7.47-7.43 (m, 2H), 7.16-7.13 (m, 2H), 7.06-7.01 (m, 2H), 6.97 (t, J = 9.5 Hz, 2H), 6.52 (s, 1H), 5.33- 5.28 (m, 1H), 4.53 (t, J = 7.9 Hz, 1H), 4.22 (d, J = 16.7 Hz, 1H), 4.11 (d, J = 16.6 Hz, 1H), 4.05-4.02 (m, 1H), 4.02-3.99 (m, 2H), 3.99-3.97 (m, 1H), 3.78 (s, 2H), 2.43-2.36 (m, 1H), 2.32 (s, 3H), 2.29-2.24 (m, 1H), 1.56 (d, J = 7.0 Hz, 3H). LC/MS (ESI) m/z: 602 (M + H)⁺. RT (Method A): 1.45 min. |

^{a}Steps 2-4 only.
^{b}HATU used in place of T₃P in Step 1.

### Scheme 30. Synthesis of (2S,4R)-N-((R)-1H-pyrrolo[3,2-c]pyridin-2-yl)ethyl)-1-(2-(5-bromo-1-oxoisoindolin-2-yl)acetyl)-4-(difluoromethoxy)pyrrolidine-2-carboxamide (Compound 100)

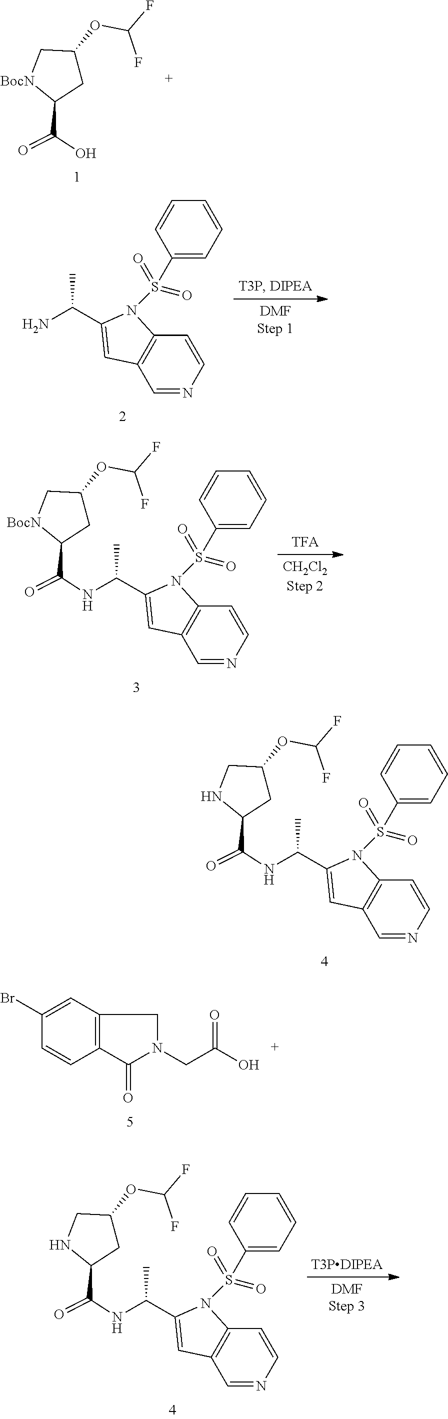 [SMILES: CC(C)(C)OC(=O)N1C[C@H](OC(F)F)C[C@H]1C(=O)O ; C[C@@H](N)c1cc2cnccc2n1S(=O)(=O)c1ccccc1 ; C[C@@H](NC(=O)[C@@H]1C[C@@H](OC(F)F)CN1C(=O)OC(C)(C)C)c1cc2cnccc2n1S(=O)(=O)c1ccccc1 ; C[C@@H](NC(=O)[C@@H]1C[C@@H](OC(F)F)CN1)c1cc2cnccc2n1S(=O)(=O)c1ccccc1 ; O=C(O)CN1Cc2cc(Br)ccc2C1=O ; C[C@@H](NC(=O)[C@@H]1C[C@@H](OC(F)F)CN1)c1cc2cnccc2n1S(=O)(=O)c1ccccc1]

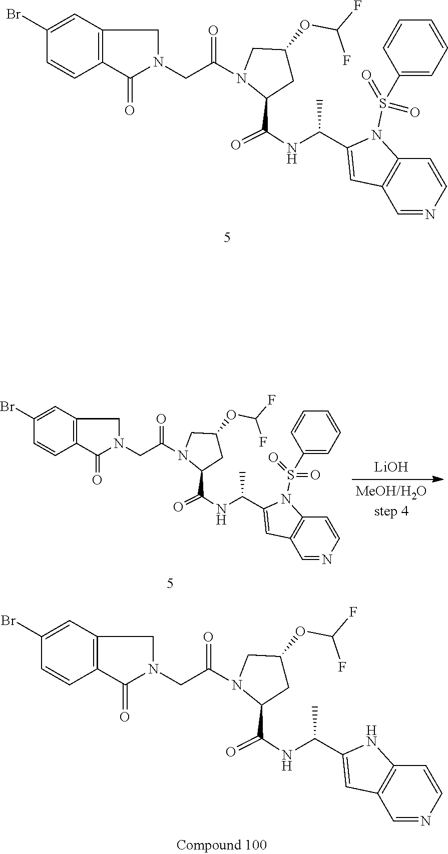

### Step 1: tert-butyl (2S,4R)-4-(difluoromethoxy)-2-(((R)-1-(1-(phenylsulfonyl)-JH-pyrrolo[3,2-c]pyridin-2-yl)ethyl)carbamoyl)pyrrolidine-1-carboxylate (3)

To a solution of (2S,4R)-1-tert-butoxycarbonyl-4-(difluoromethoxy)pyrrolidine-2-carboxylic acid (37.33 mg, 0.1327 mmol, 1.0 equiv.) in DMF (2 mL) was added (1R)-1-[1-(benzenesulfonyl)pyrrolo[3,2-c]pyridin-2-yl]ethanamine (40.00 mg, 0.1327 mmol, 1.0 equiv.), T3P, (50 mass %) in ethyl acetate (119 μL, 0.199 mmol, 1.5 equiv.), and DIPEA (69.40 μL, 0.398 mmol, 3.0 equiv.). The reaction mixture was stirred at room temperature for 30 minutes and then purified by directly by prep HPLC to give tert-butyl (2S,4R)-2-[[(1R)-1-[1-(benzenesulfonyl)pyrrolo[3,2-c]pyridin-2-yl]ethyl]carbamoyl]-4-(difluoromethoxy)pyrrolidine-1-carboxylate (64.72 mg, 0.1146 mmol, 86.37% yield). LC/MS (ESI) m/z: 565 (M+H)⁺.

### Step 2: (2S,4R)-4-(difluoromethoxy)-N-((R)-1-(1-(phenylsulfonyl)-1H-pyrrolo[3,2-c]pyridin-2-yl)ethyl)pyrrolidine-2-carboxamide (4)

TFA (0.5 mL) was added to a solution of tert-butyl (2S,4R)-2-[[(1R)-1-[1-(benzenesulfonyl)pyrrolo[3,2-c]pyridin-2-yl]ethyl]carbamoyl]-4-(difluoromethoxy) pyrrolidine-1-carboxylate (115.0 mg, 0.2037 mmol, 1.0 equiv.) in CH₂Cl₂ (1 mL) at ice-bath temperature. The reaction mixture was stirred at room temperature for 2 hours and then concentrated to dryness to give (2S,4R)-N-[(1R)-1-[1-(benzenesulfonyl)pyrrolo[3,2-c]pyridin-2-yl]ethyl]-4-(difluoromethoxy)pyrrolidine-2-carboxamide (94.00 mg, 0.2024 mmol, 99.35% yield) as a white solid, which was used in the next step without further purification. LC/MS (ESI) m/z: 465 (M+H)⁺.

### Step 3: (2S,4R)-N-[(1R)-1-[1-(benzenesulfonyl)pyrrolo[3,2-c]pyridin-2-yl]ethyl]-1-[2-(5-bromo-1-oxo-isoindolin-2-yl)acetyl]-4-(difluoromethoxy)pyrrolidine-2-carboxamide (5)

To a solution of (2S,4R)-1-[2-(5-bromo-1-oxo-isoindolin-2-yl)acetyl]-4-(difluoromethoxy) pyrrolidine-2-carboxylic acid (250.0 ng, 0.5771 mmol, 1.0 equiv.) in DMF (2 mL) was added (1R)-1-[1-(benzenesulfonyl)pyrrolo[3,2-c]pyridin-2-yl]ethanamine;dihydrochloride (0.2592 g, 0.6925 mmol, 1.2 equiv.), T₃P, (50 mass %) in ethyl acetate (0.518 mL, 0.865 mmol. 1.5 equiv.), and DIPEA (0.3020 mL, 1.73 mmol, 3.0 equiv.). The reaction mixture was stirred at room temperature for 30 minutes and then diluted with water (5 mL) and stirred for 30 minutes. The filtrate was decanted and the residue was washed with water and concentrated and then purified by combi Flash chromatography; 4 g column, solvent A=CH₂Cl₂, solvent B=MeOH to obtain (2S,4R)-N-[(1R)-1-[1-(benzenesulfonyl)pyrrolo[3,2-c]pyridin-2-yl]ethyl]-1-[2-(5-bromo-1-oxo-isoindolin-2-yl)actyl]-4-(difluoromethoxy)pyrrolidine-2-carboxamide (245.0 mg, 0.3419 mmol, 100 mass %, 59.24% yield) as a pale yellow oil. LC/MS (ESI) m/z: 717 (M+H)⁺.

### Step 4: (2S,4R)-N-((R)-1-(1H-pyrrolo[3,2-c]pyridin-2-yl)ethyl)-1-(2-(5-bromo-1-oxoisoindolin-2-yl)acetyl)-4-(difluoromethoxy)pyrrolidine-2-carboxamide (Compound 100)

To a solution of (2S,4R)-N-[(1R)-1-[1-(benzenesulfonyl)pyrrolo[3,2-c]pyridin-2-yl]ethyl]-1-[2-(5-bromo-1-oxo-isoindolin-2-yl)acetyl]-4-(difluoromethoxy)pyrrolidine-2-carboxamide (5.000 mg, 0.006977 mmol, 1.00 equiv.) in MeOH (1.5 mL) and water (0.5 mL) was added LiOH (0.5000 mg, 0.02088 mmol, 3 equiv.). The reaction mixture was stirred at room temperature overnight then concentrated to remove MeOH. The pH was adjusted to 1 using 1 N HCl. The acidified mixture concentrated and then purified by prep HPLC to give Compound 100 (1.215 mg, 0.002108 mmol, 30.21% yield) as a yellow sticky oil. LC/MS (ESI) m/z: 578 (M+H)⁺. ¹H NMR (400 MHz, DMSO-d6): δ 11.22 (s, 1H), 9.17-8.48 (m, 1H), 8.36 (s, 1H), 8.20-7.87 (m, 1H), 7.64 (t, J=9.1 Hz, 1H), 7.54-7.41 (m, 2H), 7.32-7.21 (m, 1H), 7.22-7.11 (m, 2H), 7.08-6.91 (m, 1H), 6.79 (d, J=14.4 Hz, 1H), 6.62 (s, 1H), 6.38 (s, 1H), 4.97 (s, 1H), 4.43 (t, J=6.3 Hz, 1H), 4.22-4.09 (m, 1H). 3.97-3.69 (m, 2H), 2.69-2.54 (m, 2H), 2.39-2.27 (m, 1H), 1.18 (s, 1H), 1.40 (d, J=6.9 Hz, 3H). RT (Method A): 1.17 min.

### Scheme 31. Synthesis of (2S,4R)-N-((R)-1-(1H-pyrrolo[3,2-c]pyridin-2-yl)ethyl)-4-(difluoromethoxy)-1-(2-(5-(4-fluorophenyl)-1-oxoisoindolin-2-yl)acetyl)pyrrolidine-2-carboxamide (Compound 101)

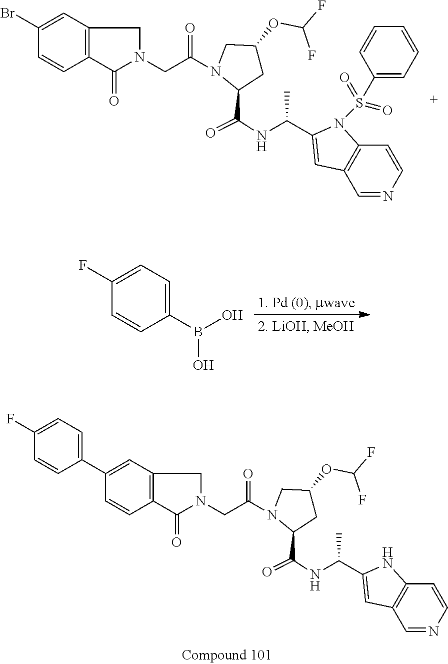

A mixture of (4-fluorophenyl)boronic acid (4.686 mg, 0.03349 mmol, 1.2 equiv.), (2S,4R)-N-[(1R)-1-[1-(benzenesulfonyl)pyrrolo[3,2-c]pyridin-2-yl]ethyl]-1-[2-(5-bromo-1-oxo-isoindolin-2-yl)acetyl]-4-(difluoromethoxy)pyrrolidine-2-carboxamide (20.00 mg, 0.02791 mmol, 1.0 equiv.), Pd(PPh₃)₂dppf·CH₂Cl₂ (2.300 mg, 0.00279 mmol.), Na₂CO₃ (5.916 mg, 0.05582 mmol, 2 equiv.) in 1,4-dioxane (3 mL), and water (0.3 mL) was stirred in a microwave reactor at 110° C. for 30 min. The reaction mixture was cooled to room temperature and then filtered through Celite pad. The cake was washed with water and EtOAc. The two layers were separated, and the aqueous layer was extracted with EtOAc, washed with brine, dried over Na₂SO₄ and concentrated to give a brown oil. To a solution of the brown oil in MeOH (1.5 mL) and water (0.5 mL) was added LiOH (1.350 mg, 0.05581 mmol, 2 equiv.). The reaction mixture was stirred at room temperature overnight and then concentrated to remove MeOH. The pH was adjusted to 1 and then the mixture was purified directly using prep HPLC to give Compound 101 (2.235 mg, 0.003778 mmol, 13.54% yield). LC/MS (ESI) m/z: 592 (M+H)⁺. ¹H NMR (400 MHz, DMSO-d6): δ 10.95 (s, 1H), 8.88 (d, J=8.1 Hz, 1H), 8.67 (d, J=8.4 Hz, 1H), 8.63 (s, 1H), 8.55 (s, 1H), 8.24 (s, 1H), 7.83 (s, 1H), 7.81-7.60 (m, 2H), 7.28 (td, J=8.8, 3.7 Hz, 1H), 7.06 (t, J=8.9 Hz, 1H), 6.96 (s, 1H), 6.68-6.52 (m, 1H), 6.32 (s, 1H), 5.05 (d, J=7.4 Hz, 1H), 4.96 (s, 1H), 4.52 (dd, J=17.2, 7.7 Hz, 2H), 4.45-4.20 (m, 3H), 3.97-3.74 (m, 2H), 2.27 (t, J=10.3 Hz, 1H), 2.11 (ddd, J=13.4, 8.2, 4.8 Hz, 1H), 1.40 (d, J=6.9 Hz, 3H). RT (Method A): 1.51 min.

The following compounds were prepared based on Scheme 31:

| Cmpd # | Reactant A | Reactant B | Characterization Data |
|---|---|---|---|
| 105 | 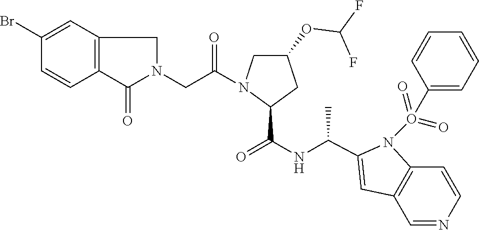 | 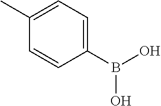 | ¹H NMR (400 MHz, DMSO-d6): δ 10.95 (s, 1H), 8.92 (d, J = 8.0 Hz, 1H), 8.68 (d, J = 8.4 Hz, 1H), 8.63 (s, 1H), 8.55 (s, 1H), 8.31 (s, 1H), 7.82 (s, 1H), 7.78-7.72 (m, 2H), 7.72-7.65 (m, 2H), 7.59 (t, J = 9.2 Hz, 1H), 7.26 (d, J = 8.0 Hz, 2H), 6.96 (s, 1H), 6.77 (s, 1H), 6.61-6.52 (m, 1H), 6.32 (s, 1H), 5.06 (t, J = 7.4 Hz, 1H), 4.96 (s, 1H), 4.69-4.12 (m, 5H), 3.92-3.70 (m, 2H), 2.11 (ddd, J = 13.3, 8.2, 4.8 Hz, 1H), 1.40 (d, J = 6.9 Hz, 3H). RT (Method A): 1.59 min. |
| 106 | 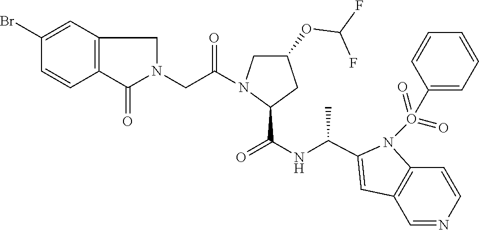 | 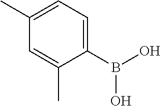 | ¹H NMR (400 MHz, DMSO-d6): δ 10.99 (s, 1H), 8.94 (s, 1H), 8.67 (d, J = 8.5 Hz, 1H), 8.57 (s, 1H), 8.31 (s, 2H), 7.88-7.62 (m, 1H), 7.52-7.37 (m, 1H), 7.23-6.99 (m, 2H), 6.77 (s, 1H), 6.70 (t, J = 6.7 Hz, 1H), 6.33 (s, 1H), 5.26-4.97 (m, 2H), 4.96 (s, 1H), 4.61-4.21 (m, 2H), 3.94-3.66 (m, 2H), 2.43-2.30 (m, 5H), 2.31 (s, 3H), 1.41 (d, J = 7.0 Hz, 3H). RT (Method A): 1.75 min. |
| 116 | 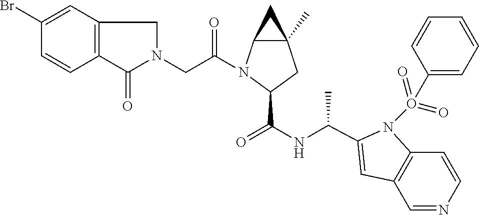 | 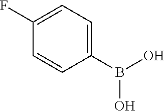 | ¹H NMR (400 MHz, DMSO-d6): δ 10.89 (s, 1H), 8.56 (s, 2H), 8.49 (d, J = 8.4 Hz, 1H), 8.32 (s, 2H), 7.88 (p, J = 8.5 Hz, 1H), 7.82-7.58 (m, 2H), 7.57-7.36 (m, 2H), 7.34-7.24 (m, 2H), 7.08 (h, J = 8.5 Hz, 2H), 6.90 (t, J = 8.8 Hz, 1H), 6.74-6.63 (m, 2H), 6.59 (s, 1H), 6.18 (s, 1H), 5.01 (t, J = 7.3 Hz, 1H), 4.77-4.59 (m, 2H), 4.55 (s, 1H), 4.41 (d, J = 16.9 Hz, 2H), 3.39 (d, J = 5.5 Hz, 1H), 2.25 (t, J = 12.3 Hz, 1H), 2.03-1.87 (m, 1H), 1.45-1.30 (m, 4H), 1.18 (d, J = 13.1 Hz, 3H), LC/MS (ESI) m/z: 552 (M + H)⁺. RT (Method A): 1.57 min. |
| 124 | 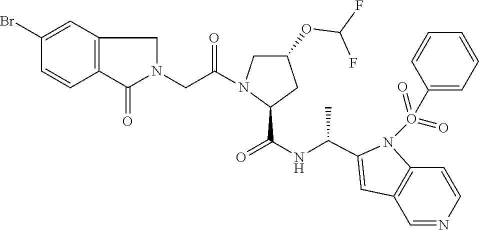 | 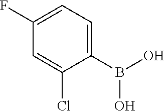 [SMILES: OB(O)c1ccc(F)cc1Cl] | ¹H NMR (400 MHz, DMSO-d6): δ 11.13 (s, 1H), 9.12-8.40 (m, 2H), 8.07 (s, 1H), 7.84 (d, J = 5.7 Hz, 1H), 7.76 (d, J = 7.8 Hz, 1H), 7.71 (d, J = 7.8 Hz, 1H), 7.59 (s, 1H), 7.53 (tt, J = 7.7, 3.8 Hz, 2H), 7.47-7.39 (m, 1H), 7.30 (td, J = 8.6, 2.6 Hz, 2H), 7.24 (d, J = 6.5 Hz, 1H), 6.95 (s, 0H), 6.82 (d, J = 5.8 Hz, 1H), 6.77 (s, 1H), 6.39 (s, 1H), 5.16-4.99 (m, 1H), 4.95 (s, 1H), 4.49 (dd, J = 17.9, 10.0 Hz, 2H), 3.92-3.73 (m, 2H), 2.27 (ddt, J = 16.1, 12.5, 5.7 Hz, 1H), 2.11 (ddd, J = 13.3, 8.0, 4.8 Hz, 1H), 1.41 (d, J = 6.9 Hz, 3H). LC/MS (ESI) m/z: 626 (M + H)⁺. RT (Method A): 1.57 min. |

### Scheme 32. Synthesis of (2S,4R)-1-((9,9-difluoro-9H-fluorene-3-carbonyl)glycyl)-4-(difluoromethoxy)-N-((S)-2-hydroxy-1-(1H-pyrrolo[3,2-c]pyridin-2-yl)ethyl) pyrrolidine-2-carboxamide (Compound 103)

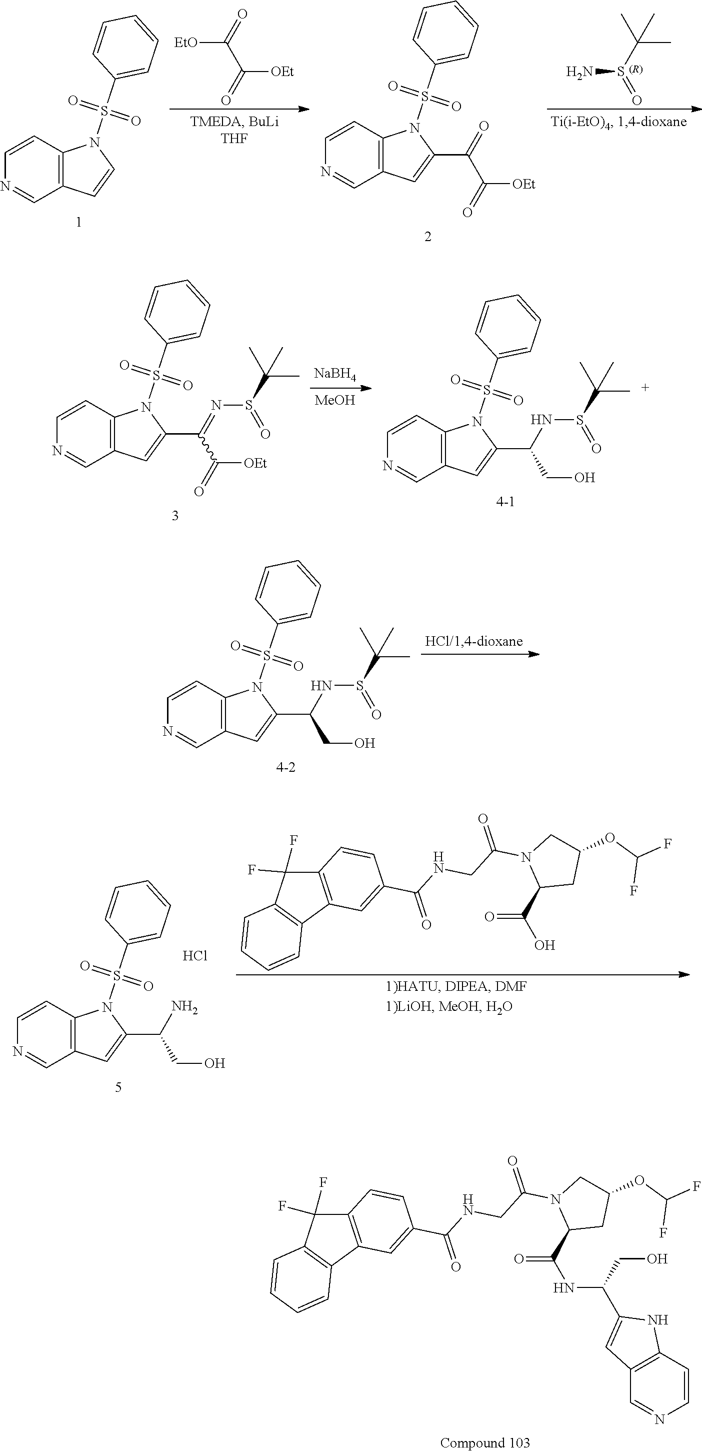

### Step 1: Ethyl 2-oxo-2-(1-(phenylsulfonyl)-1H-pyrrolo[3,2-c]pyridin-2-yl)acetate (2)

To a mixture of 1-(phenylsulfonyl)-1H-pyrrolo[3,2-c]pyridine (2.95 g, 11.4 mmol) and TMEDA (7.9 g, 68.6 mmol) in THF (30 mL) was added n-BuLi (7 mL, 2.5 M in THF) drop-wisely at −70° C. and the reaction mixture was stirred at this temperature for 2 hours. To the mixture was added diethyl oxalate (2.5 g, 17.2 mmol) drop-wisely at −70° C. and the mixture was stirred at −70° C. to 0° C. for 2 hours. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give ethyl 2-oxo-2-(1-(phenylsulfonyl)-1H-pyrrolo[3,2-c]pyridin-2-yl)acetate (1.7 g, yield 41.5%) as a yellow solid. LC/MS (ESI) m/z: 359 (M+H)⁺.

### Step 2: Ethyl (R)-2-((tert-butylsulfinyl)imino)-2-(1-(phenylsulfonyl)-1H-pyrrolo[3,2-c]pyridin-2-yl)acetate (3)

To a mixture of ethyl 2-oxo-2-(1-(phenylsulfonyl)-1H-pyrrolo[3,2-c]pyridin-2-yl)acetate (1.7 g, 4.7 mmol) and (R)-2-methylpropane-2-sulfinamide (1.0 g, 8.2 mmol) in THE (10 mL) was added Ti(OEt)₄ (2.7 g, 11.8 mmol). The mixture was stirred at 80° C. for 16 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-70% EtOAc in PE) to give ethyl (R)-2-((tert-butylsulfinyl)imino)-2-(1-(phenylsulfonyl)-1H-pyrrolo[3,2-c]pyridin-2-yl)acetate (900 mg, yield 41.1%) as a yellow solid. LC/MS (ESI) m/z: 462 (M+H)⁺.

### Step 3: (R)-N-((S)-2-hydroxy-1-(1-(phenylsulfonyl)-1H-pyrrolo[3,2-c]pyridin-2-yl)ethyl)-2-methylpropane-2-sulfinamide (4-1) and (R)-N-((R)-2-hydroxy-1-(1-(phenylsulfonyl)-1H-pyrrolo[3,2-c]pyridin-2-yl)ethyl)-2-methylpropane-2-sulfinamide (4-2)

To a solution of ethyl (R)-2-((tert-butylsulfinyl)imino)-2-(1-(phenylsulfonyl)-1H-pyrrolo[3,2-c]pyridin-2-yl)acetate (800 mg, 1.7 mmol) in MeOH (8 mL) was added NaBH₄ (1.2 g, 30.6 mmol) in portions at 0° C. and the mixture was stirred at 20° C. for 10 minutes. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel. 0-70% EtOAc in PE) to give (R)-N-((S)-2-hydroxy-1-(1-(phenylsulfonyl)-1H-pyrrolo[3,2-c]pyridin-2-yl)ethyl)-2-methylpropane-2-sulfinamide (110 mg, yield 15.4%) as a white solid. LC/MS (ESI) m/z: 422 (M+H)⁺. and (R)-N-((R)-2-hydroxy-1-(1-(phenylsulfonyl)-1H-pyrrolo[3,2-c]pyridin-2-yl)ethyl)-2-methylpropane-2-sulfinamide (210 mg, yield 29.4%) as a white solid. LC/MS (ESI) m/z: 422 (M+H)⁺.

### Step 4: (S)-2-amino-2-(1-(phenylsulfonyl)-1H-pyrrolo[3,2-c]pyridin-2-yl)ethan-1-ol hydrochloride (5)

A solution of (R)-N-((S)-2-hydroxy-1-(1-(phenylsulfonyl)-1H-pyrrolo[3,2-c]pyridin-2-yl)ethyl)-2-methylpropane-2-sulfinamide (110 mg, 0.26 mmol) in HCl/1,4-dioxane (2 mL, 4M) was stirred under N₂ atmosphere at room temperature for 2 hours. The reaction mixture was concentrated to dryness under reduced pressure to give (S)-2-amino-2-(1-(phenylsulfonyl)-1H-pyrrolo[3,2-c]pyridin-2-yl)ethan-1-ol hydrochloride (80 mg, yield 87.0%) as a yellow solid, which was used directly in the next step without further purification. LC/MS (ESI) m/z: 318 (M+H)⁺.

### Step 5: (2S,4R)-1-((9,9-difluoro-9H-fluorene-3-carbonyl)glycyl)-4-(difluoromethoxy)-N-((S)-2-hydroxy-1-(1H-pyrrolo[3,2-c]pyridin-2-yl)ethyl)pyrrolidine-2-carboxamide (Compound 103)

To a mixture of (S)-2-amino-2-(1-(phenylsulfonyl)-1H-pyrrolo[3,2-c]pyridin-2-yl)ethan-1-ol hydrochloride (23 mg, 0.067 mmol) and (2S,4R)-1-((9,9-difluoro-9H-fluorene-3-carbonyl)glycyl)-4-(difluoromethoxy)pyrrolidine-2-carboxylic acid (30 mg, 0.067 mmol) in DMF (1 mL) was added DIPEA (26 mg, 0.20 mmol) and HATU (38 mg, 0.10 mmol) under N₂ atmosphere and the reaction mixture was stirred at room temperature for 1 hours. To the mixture was added LiOH (4.8 mg, 0.20 mmol), MeOH (0.5 mL) and water (0.1 mL) and the mixture was stirred at room temperature for 6 hours. The mixture was diluted with EtOAc and washed with saturated aq. NaHCO₃ solution and brine, dried over anhydrous Na₂SO₄, filtered, and concentrated under reduced pressure to dryness. The residue was purified by prep-TLC (DCM:MeOH=7:1) and further purified by prep-HPLC to give Compound 103 (2.0 mg, yield 4.8%) as a white solid. ¹H NMR (400 MHz, CD₃OD): δ 8.82 (s, 1H), 8.45 (s, 1H), 8.21 (s, 1H), 7.92 (d, J=6.9 Hz, 2H), 7.76-7.70 (m, 2H), 7.67 (d, J=7.4 Hz, 1H), 7.56 (t, J=7.2 Hz, 1H), 7.44 (dd, J=12.1, 6.2 Hz, 2H), 6.84 (s, 1H), 6.55 (t, J=74.8 Hz, 1H), 5.30 (t, J=5.9 Hz, 1H), 5.08 (s, 1H), 4.69 (t, J=8.1 Hz, 1H), 4.37 (d, J=16.9 Hz, 1H), 4.22 (d, J=16.6 Hz, 1H), 4.01 (dd, J=10.1, 4.9 Hz, 2H), 3.93 (dd, J=11.3, 4.9 Hz, 2H), 2.52 (dd, J=14.7, 8.5 Hz, 1H), 2.34 (dd, J=12.5, 6.2 Hz, 1H). LC/MS (ESI) m/z: 626 (M+H)⁺. RT (Method A): 1.38 min.

### Scheme 33. Synthesis of (S)-N-((1H-pyrrolo[3,2-c]pyridin-2-yl)methyl)-7-((5-phenylpicolinoyl)glycyl)-1,4-dioxa-7-azaspiro[4.4]nonane-8-carboxamide (Compound 107)

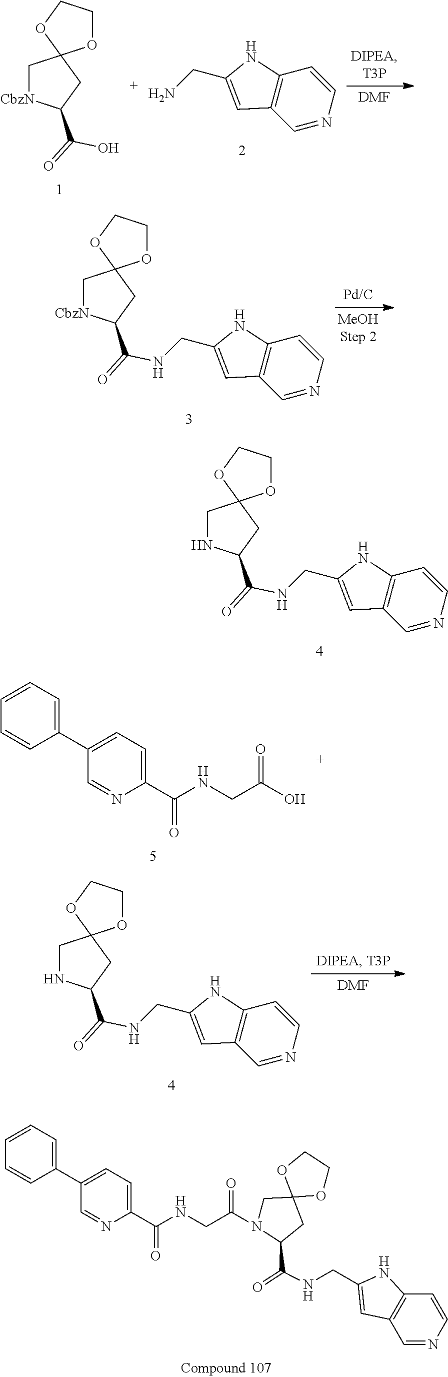

### Step 1: benzyl (8S)-8-(1H-pyrrolo[3,2-c]pyridin-2-ylmethylcarbamoyl)-1,4-dioxa-7-azaspiro[4.4]nonane-7-carboxylate (3)

To a solution of (7S)-6-benzyloxycarbonyl-1,4-dioxa-6-azaspiro[4.4]nonane-7-carboxylic acid (50.00 mg, 0.1627 mmol, 1.0 equiv.) in DMF (2 mL) was added 1H-pyrrolo[3,2-c]pyridin-2-ylmethanamine dihydrochloride (39.39 mg, 0.1790 mmol, 1.0 equiv.), T3P (50 mass %) in ethyl acetate (0.146 mL, 0.244 mmol, 1.5 equiv.), and DIPEA (85.1 μL, 0.488 mmol, 3.0 equiv.). The reaction mixture was stirred at room temperature for 30 minutes and then purified directly prep HPLC to give benzyl (8S)-8-(1H-pyrrolo[3,2-c]pyridin-2-ylmethylcarbamoyl)-1,4-dioxa-7-azaspiro[4.4]nonane-7-carboxylate (56.92 mg, 0.1304 mmol, 80.14% yield). LC/MS (ESI) m/z: 437 (M+H)⁺.

### Step 2: (8S)-N-(1H-pyrrolo[3,2-c]pyridin-2-ylmethyl)-1,4-dioxa-7-azaspiro[4.4]nonane-8-carboxamide (4)

To a solution of benzyl (8S)-8-(1H-pyrrolo[3,2-c]pyridin-2-ylmethylcarbamoyl)-1,4-dioxa-7-azaspiro[4.4]nonane-7-carboxylate (30.00 mg, 0.06873 mmol, 1.0 equiv.) in MeOH (2 mL) was added 10% Pd/C (14.63 mg, 0.006874 mmol, 0.1 equiv.). The flask was evacuated and then backfilled with Hydrogen in a balloon. The reaction mixture was stirred at room temperature overnight and then filtered through Celite pad. The filtrate was concentrated and dried in vacuo to give (8S)-N-(1H-pyrrolo[3,2-c]pyridin-2-ylmethyl)-1,4-dioxa-7-azaspiro[4.4]nonane-8-carboxamide (18.56 mg, 0.06140 mmol, 100 mass %, 89.33% yield). LC/MS (ESI) m/z: 303 (M+H)⁺.

### Step 3: (S)-N-((JH-pyrrolo[3,2-c]pyridin-2-yl)methyl)-7-((5-phenylpicolinoyl)glycyl)-1,4-dioxa-7-azaspiro[4.4]nonane-8-carboxamide (Compound 107)

To a solution of 2-[(5-phenylpyridine-2-carbonyl)amino]acetic acid (7.565 mg, 0.02952 mmol, 1.0 equiv.) in DMF (2 mL) was added (8S)-N-(1H-pyrrolo[3,2-c]pyridin-2-ylmethyl)-1,4-dioxa-7-azaspiro[4.4]nonane-8-carboxamide;hydrochloride (10.00 mg, 0.02952 mmol, 1.0 equiv.), T3P (50 mass %) in ethyl acetate (26.5 μL, 0.0443 mmol, 1.5 equiv.), and DIPEA (15.4 μL, 0.0883 mmol, 3.0 equiv.). The reaction mixture was stirred at room temperature for 30 minutes and then purified directly prep HPLC to give Compound 107 (2.917 mg, 0.005396 mmol, 18.28% yield). LC/MS (ESI) m/z: 541 (M+H)⁺. ¹H NMR (400 MHz, DMSO-d6): δ 11.08 (s, 1H), 8.94 (d, J=4.6 Hz, 1H), 8.83-8.70 (m, 1H), 8.63 (d, J=14.3 Hz, 1H), 8.47 (t, J=5.8 Hz, 1H), 8.23 (dd, J=7.9, 2.6 Hz, 1H), 8.12 (d, J=3.6 Hz, 1H), 8.05 (dd, J=8.8, 5.6 Hz, 1H), 7.96 (d, J=5.5 Hz, 1H), 7.76 (d, J=7.5 Hz, 3H), 7.49 (t, J=6.8 Hz, 3H), 7.43 (d, J=6.8 Hz, 1H), 7.24 (d, J=5.6 Hz, 1H), 7.15 (d, J=5.6 Hz, 1H), 6.71-6.21 (m, 3H), 4.91-4.28 (m, 2H), 4.14 (dd, J=12.5, 5.3 Hz, 2H), 4.00-3.77 (m, 6H), 3.73 (d, J=10.8 Hz, 1H), 3.59 (d, J=10.7 Hz, 1H), 2.28 (dd, J=13.1, 8.6 Hz, 1H), 2.08 (dd, J=13.0, 6.8 Hz, 1H). RT (Method A): 1.20 min.

### Scheme 34. Synthesis of (S)-N-((1H-pyrrolo[3,2-c]pyridin-2-yl)methyl)-7-((3-fluoro-5-(p-tolyl)picolinoyl)glycyl)-1,4-dioxa-7-azaspiro[4.4]nonane-8-carboxamide (Compound 108)

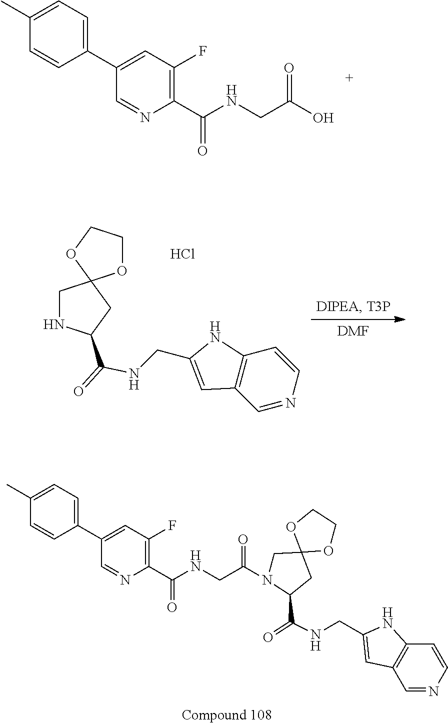

To a solution of 2-[[3-fluoro-5-(p-tolyl)pyridine-2-carbonyl]amino]acetic acid (8.509 mg, 0.02951 mmol, 1.0 equiv.) in DMF (2 mL) was added (8S)-N-(1H-pyrrolo[3,2-c]pyridin-2-ylmethyl)-1,4-dioxa-7-azaspiro[4.4]nonane-8-carboxamide;hydrochloride (10.00 mg, 0.02952 mmol, 1.0 equiv.), T3P (50 mass %) in ethyl acetate (26.5 μL, 0.0443 mmol, 1.5 equiv.), and DIPEA (15.4 NL, 0.0883 mmol, 3.0 equiv.). The reaction mixture was stirred at room temperature for 30 minutes and then purified directly prep HPLC to give Compound 108 (3.857 mg, 0.006736 mmol, 22.82% yield). LC/MS (ESI) m/z: 573 (M+H)⁺. ¹H NMR (400 MHz, DMSO-d6): δ 11.11 (s, 1H), 8.80 (s, 1H), 8.65 (dd, J=12.7, 8.0 Hz, 2H), 8.47 (d, J=5.9 Hz, 1H), 8.13 (d, J=13.8 Hz, 1H), 7.97 (d, J=5.6 Hz, 1H), 7.72 (d, J=7.8 Hz, 3H), 7.30 (d, J=7.7 Hz, 3H), 7.18 (d, J=5.5 Hz, 1H), 6.40 (s, 1H), 4.38 (qd, J=15.8, 7.7 Hz, 2H), 4.09 (qd, J=17.4, 5.3 Hz, 2H), 3.95-3.77 (m, 3H), 3.73 (d, J=10.7 Hz, 1H), 3.59 (d, J=10.7 Hz, 1H), 3.34 (d, J=12.1 Hz, 1H), 2.32 (s, 3H), 2.07 (dd, J=13.1, 6.8 Hz, 2H). RT (Method A): 1.31 min.

The following compounds were prepared based on Scheme 34:

| Cmpd # | Reactant A | Reactant B | Characterization Data |
|---|---|---|---|
| 117 | 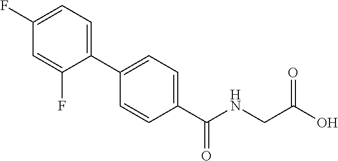 [SMILES: O=C(O)CNC(=O)c1ccc(-c2ccc(F)cc2F)cc1] | 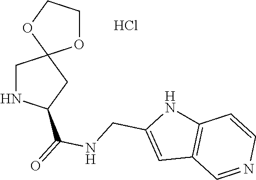 [SMILES: O=C(NCc1cc2cnccc2[nH]1)[C@@H]1CC2(CN1)OCCO2 ; Cl] | ¹H NMR (400 MHz, DMSO-d6): δ 11.10 (s, 1H), 8.13 (s, 1H), 7.93 (dd, J = 12.9, 6.7 Hz, 4H), 7.58 (t, J = 7.2 Hz, 4H), 7.33 (td, J = 10.3, 2.6 Hz, 1H), 7.25 (s, 1H), 7.17 (td, J = 8.4, 2.5 Hz, 2H), 7.11 (d, J = 5.4 Hz, 1H), 6.39 (s, 1H), 6.33 (s, 1H), 4.67-4.24 (m, 3H), 4.15 (dd, J = 16.8, 5.5 Hz, 1H), 3.88 (dp, J = 16.4, 5.2 Hz, 2H), 3.76 (d, J = 10.9 Hz, 1H), 3.60 (d, J = 10.9 Hz, 1H), 2.26 (dd, J = 13.2, 8.7 Hz, 1H), 2.06 (dd, J = 13.0, 7.2 Hz, 1H). LC/MS (ESI) m/z: 576 (M + H)⁺. RT (Method A): 1.35 min. |
| 125 | 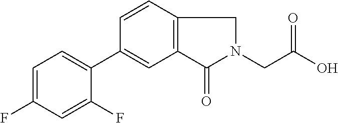 [SMILES: O=C(O)CN1Cc2ccc(-c3ccc(F)cc3F)cc2C1=O] | 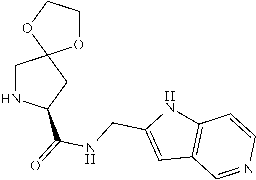 [SMILES: O=C(NCc1cc2cnccc2[nH]1)[C@@H]1CC2(CN1)OCCO2] | ¹H NMR (400 MHz, DMSO-d6): δ 11.07 (s, 1H), 8.66-8.36 (m, 2H), 8.20 (s, 0H), 7.91 (d, J = 5.5 Hz, 1H), 7.75 (d, J = 7.9 Hz, 1H), 7.68 (s, 1H), 7.59 (dd, J = 9.6, 7.2 Hz, 2H), 7.35 (ddd, J = 11.4, 9.3, 2.6 Hz, 1H), 7.18 (td, J = 8.4, 2.5 Hz, 1H), 7.00 (d, J = 5.6 Hz, 1H), 6.31 (s, 1H), 4.59-4.39 (m, 3H), 4.40-4.22 (m, 4H), 3.89 (dtt, J = 15.0, 9.8, 4.1 Hz, 5H), 3.76 (d, J = 10.8 Hz, 1H), 3.60 (dd, J = 11.5, 5.1 Hz, 1H), 2.26 (td, J = 11.4, 6.6 Hz, 1H), 2.04 (dd, J = 13.1, 7.4 Hz, 1H). LC/MS (ESI) m/z: 588 (M + H)⁺. RT (Method A): 1.35 min. |
| 129 | 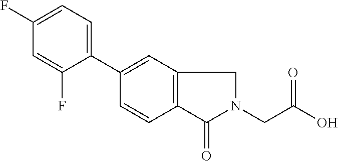 [SMILES: O=C(O)CN1Cc2cc(-c3ccc(F)cc3F)ccc2C1=O] | 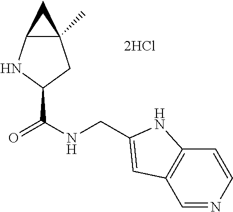 | ¹H NMR (400 MHz, DMSO-d6): δ 11.13 (s, 1H), 8.59 (s, 1H), 8.45 (t, J = 5.6 Hz, 1H), 8.27 (s, 1H), 7.92 (d, J = 5.6 Hz, 1H), 7.75 (d, J = 8.0 Hz, 1H), 7.59 (q, J = 8.3 Hz, 2H), 7.45-7.29 (m, 1H), 7.18 (td, J = 8.6, 2.5 Hz, 1H), 7.01 (d, J = 5.6 Hz, 1H), 6.28 (s, 1H), 4.81-4.52 (m, 3H), 4.43 (dd, J = 17.1, 11.6 Hz, 2H), 4.34 (t, J = 6.1 Hz, 2H), 2.25 (t, J = 12.3 Hz, 1H), 1.31-0.97 (m, 5H), 0.61 (t, J = 5.5 Hz, 1H). LC/MS (ESI) m/z: 556 (M + H)⁺. RT (Method A): 1.54 min. |
| 136 | 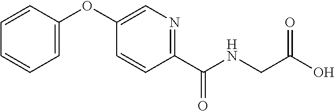 [SMILES: O=C(O)CNC(=O)c1ccc(Oc2ccccc2)cn1] | 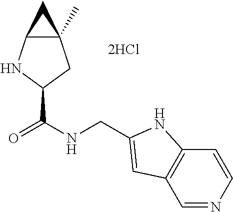 | ¹H NMR (400 MHz, DMSO-d6): δ 11.15 (s, 1H), 8.63 (d, J = 10.4 Hz, 2H), 8.49- 8.33 (m, 2H), 8.28 (s, 1H), 7.96 (dd, J = 9.1, 5.2 Hz, 2H), 7.50-7.29 (m, 4H), 7.28-7.13 (m, 3H), 6.29 (d, J = 6.0 Hz, 1H), 4.66 (dd, J = 11.2, 3.8 Hz, 1H), 4.33 (t, J = 7.8 Hz, 3H), 4.13 (dd, J = 16.9, 5.9 Hz, 1H), 2.25 (t, J = 12.1 Hz, 1H), 2.00 (d, J = 11.6 Hz, 1H), 1.17 (q, J = 9.7 Hz, 5H), 0.66 (t, J = 5.8 Hz, 1H). LC/MS (ESI) m/z: 525 (M + H)⁺. RT (Method A): 1.46 min. |
| 144 | 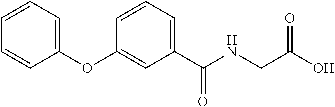 [SMILES: O=C(O)CNC(=O)c1cccc(Oc2ccccc2)c1] | 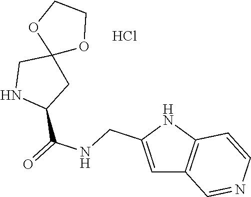 [SMILES: O=C(NCc1cc2cnccc2[nH]1)[C@@H]1CC2(CN1)OCCO2 ; Cl] | ¹H NMR (400 MHz, DMSO-d6): δ 11.20 (s, 1H), 8.67 (dd, J = 13.5, 7.5 Hz, 3H), 8.45 (d, J = 5.8 Hz, 1H), 8.07 (s, 0H), 7.99 (s, 1H), 7.60 (d, J = 7.6 Hz, 1H), 7.47- 7.37 (m, 3H), 7.33 (t, J = 7.7 Hz, 2H), 7.23-7.04 (m, 4H), 6.96 (d, J = 7.9 Hz, 3H), 6.35 (s, 1H), 4.64 (s, 0H), 4.57-4.36 (m, 2H), 4.10 (dd, J = 16.7, 5.7 Hz, 1H), 4.04-3.79 (m, 6H), 3.57 (d, J = 10.9 Hz, 1H), 2.36-2.13 (m, 1H), 2.04 (dd, J = 13.1, 7.3 Hz, 1H), 0.86 (ddd, J = 16.1, 7.7, 4.6 Hz, 2H). LC/MS (ESI) m/z: 556 (M + H)⁺. RT (Method A): 1.28 min. |
| 152 | 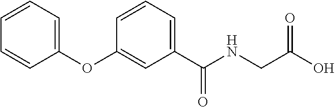 [SMILES: O=C(O)CNC(=O)c1cccc(Oc2ccccc2)c1] | 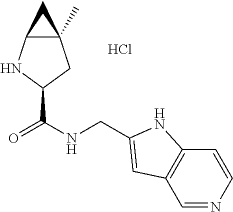 | ¹H NMR (400 MHz, DMSO-d6): δ 11.50 (s, 1H), 8.71 (t, J = 5.7 Hz, 2H), 8.40 (t, J = 5.6 Hz, 1H), 8.05 (d, J = 13.1 Hz, 1H), 7.59 (d, J = 7.6 Hz, 1H), 7.52-7.37 (m, 3H), 7.33 (t, J = 7.9 Hz, 2H), 7.28 (s, 1H), 7.18-7.04 (m, 2H), 6.96 (d, J = 8.1 Hz, 2H), 6.39 (s, 1H), 4.64 (dd, J = 11.2, 3.3 Hz, 1H), 4.52-4.17 (m, 3H), 3.98 (dd, J = 16.5, 5.6 Hz, 1H), 3.36 (dd, J = 5.9, 2.4 Hz, 1H), 2.36-2.11 (m, 1H), 1.98 (dd, J = 13.2, 3.3 Hz, 1H), 1.41 (qt, J = 6.5, 4.1 Hz, 3H), 1.28-1.01 (m, 5H), 0.97-0.74 (m, 1H), 0.63 (t, J = 5.5 Hz, 1H). LC/MS (ESI) m/z: 524 (M + H)⁺. RT (Method A): 1.49 min. |
| 155 | 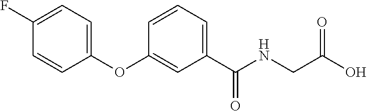 [SMILES: O=C(O)CNC(=O)c1cccc(Oc2ccc(F)cc2)c1] | 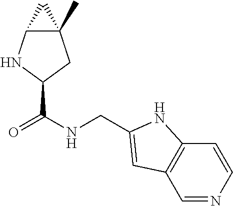 | ¹H NMR (400 MHz, DMSO-d6): δ 11.88 (s, 1H), 8.84 (s, 1H), 8.51 (t, J = 5.6 Hz, 1H), 8.18-8.09 (m, 1H), 8.07 (s, 1H), 7.54 (t, J = 7.2 Hz, 1H), 7.47-7.30 (m, 3H), 7.25 (dd, J = 8.2, 2.5 Hz, 1H), 7.19-7.05 (m, 3H), 6.51 (s, 1H), 6.42 (s, 2H), 4.79-4.45 (m, 3H), 3.42-3.29 (m, 16H), 2.25 (dd, J = 13.8, 10.9 Hz, 1H), 1.96 (dd, J = 13.3, 3.3 Hz, 1H), 1.02 (s, 3H). LC/MS (ESI) m/z: 542 (M + H)⁺. RT (Method A): 1.50 min. |
| 156 | 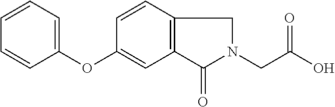 [SMILES: O=C(O)CN1Cc2ccc(Oc3ccccc3)cc2C1=O] | 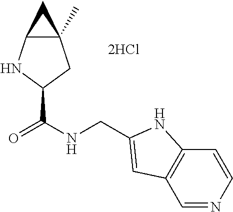 | ¹H NMR (400 MHz, DMSO-d6): δ 11.88 (s, 1H), 8.84 (s, 1H), 8.51 (s, 1H), 8.16- 8.09 (m, 1H), 8.07 (s, 1H), 7.54 (t, J = 7.2 Hz, 1H), 7.43-7.30 (m, 3H), 7.25 (dd, J = 8.2, 2.5 Hz, 1H), 7.14-7.04 (m, 2H), 7.00 (d, J = 8.0 Hz, 2H), 6.51 (s, 1H), 6.42 (s, 2H), 4.77-4.45 (m, 3H), 4.43-4.23 (m, 4H), 2.43 (t, J = 1.9 Hz, 4H), 2.25 (t, J = 12.3 Hz, 1H), 1.96 (dd, J = 13.3, 3.3 Hz, 1H), 1.29-1.03 (m, 1H). LC/MS (ESI) m/z: 536 (M + H)⁺. RT (Method A): 1.52 min. |
| 157 | 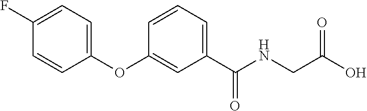 [SMILES: O=C(O)CNC(=O)c1cccc(Oc2ccc(F)cc2)c1] | 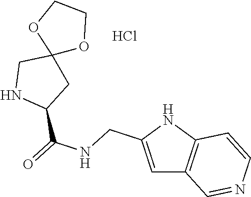 [SMILES: O=C(NCc1cc2cnccc2[nH]1)[C@@H]1CC2(CN1)OCCO2 ; Cl] | ¹H NMR (400 MHz, DMSO-d6): δ 11.09 (s, 1H), 8.67 (dd, J = 13.7, 7.8 Hz, 1H), 8.45 (t, J = 5.8 Hz, 1H), 8.25 (s, 1H), 7.96 (d, J = 5.6 Hz, 1H), 7.59 (d, J = 7.6 Hz, 1H), 7.49-7.33 (m, 1H), 7.19 (dt, J = 17.4, 7.2 Hz, 1H), 7.12 (dd, J = 8.0, 2.6 Hz, 2H), 7.04 (td, J = 8.8, 4.5 Hz, 3H), 6.54-5.82 (m, 1H), 4.56-4.19 (m, 2H), 4.10 (dd, J = 16.7, 5.7 Hz, 1H), 3.89 (tdd, J = 15.8, 10.4, 6.0 Hz, 1H), 3.73 (d, J = 10.9 Hz, 1H), 3.57 (d, J = 10.8 Hz, 1H), 2.30-2.16 (m, 1H), 2.04 (dd, J = 13.0, 7.3 Hz, 1H), 1.30-1.07 (m, 2H), 0.98-0.67 (m, 1H). LC/MS (ESI) m/z: 574 (M + H)⁺. RT (Method A): 1.31 min. |
| 159 | 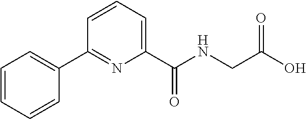 [SMILES: O=C(O)CNC(=O)c1cccc(-c2ccccc2)n1] | 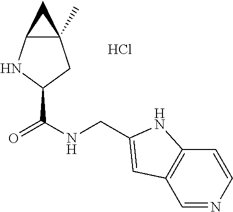 | ¹H NMR (400 MHz, DMSO-d6): δ 11.22 (s, 1H), 9.20-8.95 (m, 1H), 8.81 (s, 1H), 8.73 (s, 1H), 8.63 (s, 1H), 8.55 (t, J = 5.7 Hz, 1H), 8.38-8.20 (m, 2H), 8.15- 8.05 (m, 1H), 7.99 (d, J = 7.5 Hz, 1H), 7.88 (d, J = 5.7 Hz, 1H), 7.61-7.45 (m, 2H), 7.31 (s, 1H), 7.13 (d, J = 5.6 Hz, 1H), 6.35 (s, 1H), 4.74 (dd, J = 11.3, 3.4 Hz, 1H), 4.60-4.29 (m, 4H), 4.24 (dd, J = 17.0, 5.6 Hz, 1H), 2.34 (t, J = 12.2 Hz, 1H), 2.07 (dd, J = 13.3, 3.5 Hz, 1H), 1.26 (s, 3H). LC/MS (ESI) m/z: 509 (M + H)⁺. RT (Method A): 1.41 min. |
| 239 | 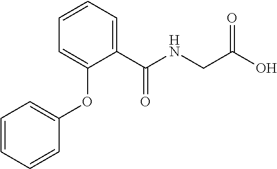 [SMILES: O=C(O)CNC(=O)c1ccccc1Oc1ccccc1] | 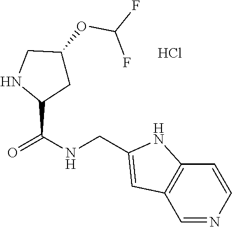 | ¹H NMR (400 MHz, DMSO-d6): δ 11.22 (s, 1H), 8.74-8.55 (m, 1H), 8.50 (t, J = 4.5 Hz, 1H), 8.19 (s, 1H), 8.01 (dd, J = 10.8, 5,6 Hz, 1H), 7.84 (dd, J = 12.6, 7.6 Hz, 1H), 7.42 (t, J = 7.9 Hz, 1H), 7.35 (dt, J = 13.1, 8.0 Hz, 1H), 7.25-7.08 (m, 2H), 7.03 (t, J = 8.2 Hz, 2H), 6.87-6.72 (m, 2H), 6.33 (s, 1H), 4.87 (s, 1H), 4.54- 4.18 (m, 3H), 4.10 (qd, J = 17.4, 5.2 Hz, 2H), 3.78 (d, J = 13.3 Hz, 2H), 3.66 (d, J = 10.5 Hz, 1H), 3.52 (d, J = 13.2 Hz, 1H), 2.23 (d, J = 11.2 Hz, 1H), 2.19-1.88 (m, 1H). LC/MS (ESI) m/z: 564 (M + H)⁺. RT (Method A): 1.36 min. |
| 241 | 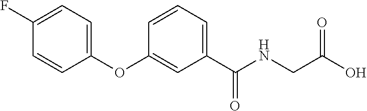 [SMILES: O=C(O)CNC(=O)c1cccc(Oc2ccc(F)cc2)c1] | 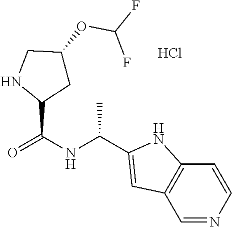 | ¹H NMR (400 MHz, DMSO-d6): δ 11.15 (s, 1H), 8.86 (d, J = 5.8 Hz, 1H), 8.79- 8.58 (m, 2H), 8.44 (s, 1H), 7.92 (d, J = 5.6 Hz, 1H), 7.71 (d, J = 7.7 Hz, 1H), 7.58- 7.46 (m, 2H), 7.33-7.20 (m, 2H), 7.11 (td, J = 10.3, 4.4 Hz, 3H), 6.93 (s, 2H), 6.83 (s, 1H), 6.41 (s, 1H), 6.30 (s, 1H), 5.15 (dt, J = 15.3, 7.7 Hz, 1H), 4.66 (t, J = 7.3 Hz, 1H), 4.43 (t, J = 7.8 Hz, 1H), 4.22 (dd, J = 16.8, 5.9 Hz, 1H), 4.02 (dd, J = 16.8, 5.7 Hz, 1H), 3.97-3.67 (m, 2H), 2.31 (d, J = 14.2 Hz, 1H), 2.25-1.99 (m, 1H), 1.47 (d, J = 7.0 Hz, 3H). LC/MS (ESI) m/z: 596 (M + H)⁺. RT (Method A): 1.38 min. |
| 284^{a,b} | 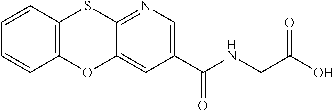 [SMILES: O=C(O)CNC(=O)c1cnc2c(c1)Oc1ccccc1S2] | 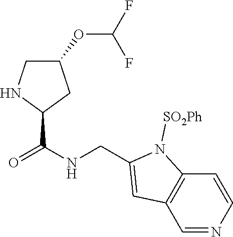 | ¹H-NMR (400 MHz, DMSO-d6): δ 8.90 (s, 1H), 8.71-8.49 (m, 2H), 8.38 (s, 1H), 7.96 (d, J = 6.1 Hz, 1H), 7.71 (d, J = 8.9 Hz, 1H), 7.27-7.04 (m, 3H), 6.71 (m, 1H), 6.35 (m, 1H), 4.90 (s, 1H), 1.68 (q, J = 5.9 Hz, 2H), 1.49 (m, 1H), 1.24 (d, J = 8.2 Hz, 1H). LC/MS (ESI) m/z: 595 (M + H)⁺. RT (Method A): 1.26 min. |

^{a}HATU was used in place of T₃P.
^{b}The coupling reaction was followed by deprotection by the addition of KOH and TBAI.

### Scheme 35. Synthesis of 7-{2-[(Phenoxathiine-3-carbonyl)-amino]-acetyl}-1,4-dioxa-7-aza-spiro[4.4]nonane-8-carboxylic acid (pyrrolo[1,2-a]pyrazin-7-ylmethyl)-amide (Compound 111)

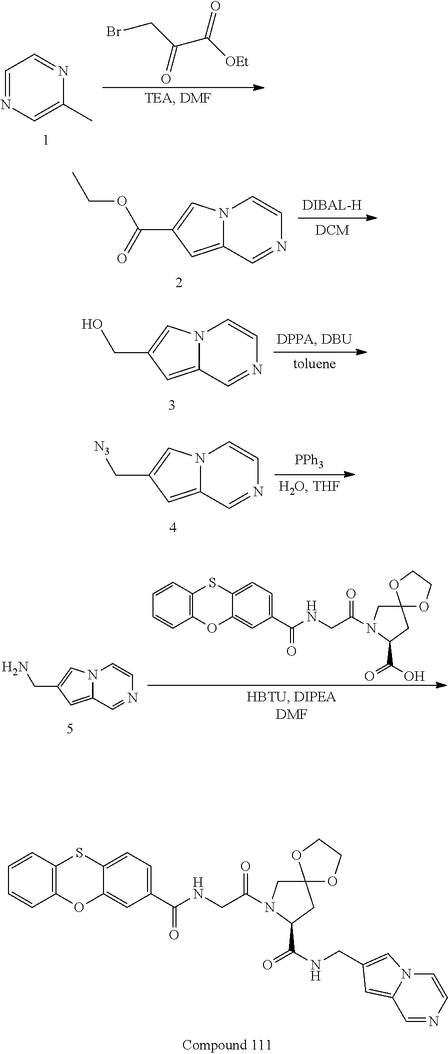

### Step 1: Pyrrolo[1,2-a]pyrazine-7-carboxylic acid ethyl ester (2)

To a mixture of 2-Methyl-pyrazine (9.0 g, 95.63 mmol) and 3-Bromo-2-oxo-propionic acid ethyl ester (18.65 g, 95.63 mmol) in DMF (130 mL) and the mixture was stirred at 90° C. for 1 day in a sealed tube. The reaction mixture was cooled to room temperature for 0° C., TEA (28.98 g, 286.93 mmol) was added to the reaction mixture and stirred at 60° C. for 2 hours. The mixture was diluted with EtOAc and filtered. The filtrate was washed with saturated aq. NaHCO₃ solution and brine, dried over anhydrous Na₂SO₄, filtered, and concentrated under reduced pressure to dryness. The residue was purified by flash chromatography (silica gel, 0-60% EtOAc in PE) to give Pyrrolo[1,2-a]pyrazine-7-carboxylic acid ethyl ester (650 mg, yield 18.2%) as a yellow oil. LC/MS (ESI) m/z: 191 (M+H)⁺.

### Step 2: Pyrrolo[1,2-a]pyrazin-7-yl-methanol (3)

To a solution of Pyrrolo[1,2-a]pyrazine-7-carboxylic acid ethyl ester (600 mg, 0.3.16 mmol) in DCM (10 mL) was added DIBAL-H (9.1 mL, 9.1 mmol, 1 N in THF) drop-wisely under N₂ atmosphere at 0° C. and the mixture was stirred at 27° C. for 4 hours. The mixture was quenched with saturated aq. potassium sodium tartrate solution and extracted with DCM twice. The combined organic layers were washed with brine, dried over anhydrous Na₂SO₄, filtered, and concentrated under reduced pressure to dryness. The residue was purified by flash chromatography (silica gel, 0-20% MeOH in DCM) to give Pyrrolo[1,2-a]pyrazin-7-yl-methanol (45 mg, yield 9.6%) as a yellow solid. LC/MS (ESI) m/z: 149 (M+H)⁺.

### Step 3: 7-Azidomethyl-pyrrolo[1,2-a]pyrazine (4)

To a solution of Pyrrolo[1,2-a]pyrazin-7-yl-methanol (45 mg, 0.30 mmol) in toluene (3 mL) was added DPPA (84 mg, 0.31 mmol) and DBU (93 mg, 0.61 mmol) under N₂ atmosphere. The mixture was stirred at 110° C. for 2 hours. The reaction mixture was diluted with EtOAc and filtered. The filtrate was washed with saturated aq. NaHCO₃ solution and brine, dried over anhydrous Na₂SO₄, filtered, and concentrated under reduced pressure to dryness. The residue was purified by prep-TLC (DCM:MeOH=20:1) to give 7-Azidomethyl-pyrrolo[1,2-a]pyrazine (40 mg, yield 76.1%) as a light oil. LC/MS (ESI) m/z: 174 (M+H)⁺.

### Step 4: C-Pyrrolo[, 2-a]pyrazin-7-yl-methylamine (5)

To a solution of 7-Azidomethyl-pyrrolo[1,2-a]pyrazine (40 mg, 0.23 mmol) in THF (2 mL) and water (2 mL) was added PPh₃ (90 mg, 0.34 mmol) under N₂ atmosphere. The mixture was stirred at 40° C. for 2 hours. The reaction mixture was concentrated under reduced pressure to dryness. The residue was purified by prep-TLC (DCM:MeOH=10:1) to give C-Pyrrolo[1,2-a]pyrazin-7-yl-methylamine (20 mg, yield 58.8%) as a light oil. LC/MS (ESI) m/z: 148 (M+H)⁺.

### Step 5: 7-{2-[(Phenoxathiine-3-carbonyl)-amino]-acetyl}-1,4-dioxa-7-aza-spiro[4.4]nonane-8-carboxylic acid (pyrrolo[1,2-a]pyrazin-7-ylmethyl)-amide (Compound III)

To a mixture of C-Pyrrolo[1,2-a]pyrazin-7-yl-methylamine (20 mg, 0.14 mmol) and 7-{2-[(Phenoxathiine-3-carbonyl)-amino]-acetyl}-1,4-dioxa-7-aza-spiro[4.4]nonane-8-carboxylic acid (62 mg, 0.14 mmol) in DMF (3 mL) was added DIPEA (88 mg, 0.68 mmol) and HBTU (77 mg, 0.20 mmol) was added to the stirring mixture and the resulting mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 111 (2.3 mg, yield 2.9%) as a white solid. ¹H NMR (400 MHz, CD₃OD): δ 8.52 (s, 1H), 7.89 (d, J=4.7 Hz, 1H), 7.57 (s, 1H), 7.45-7.41 (m, 1H), 7.36-7.32 (m, 1H), 7.22-7.17 (m, 2H), 7.16-7.10 (m, 2H), 7.09-7.06 (m, 1H), 7.02 (d, J=8.0 Hz, 1H), 6.81 (s, 1H), 4.65-4.62 (m, 1H), 4.61-4.58 (m, 1H), 4.53-4.47 (m, 1H), 4.17 (d, J=16.4 Hz, 1H), 4.10-4.04 (m, 1H), 4.01 (s, 4H), 3.87-3.78 (m, 2H), 2.45 (dd, J=13.2, 9.2 Hz, 1H), 2.25 (dd, J=13.2, 5.5 Hz, 1H). LC/MS (ESI) m/z: 586 (M+H)⁺. RT (Method A): 1.27 min.

### Scheme 36. Synthesis of (1S,3S,5S)-5-methyl-2-((phenoxathiine-3-carbonyl)glycyl)-N-((4,5,6,7-tetrahydrothieno[2,3-c]pyridin-2-yl)methyl)-2-azabicyclo[3.1.0]hexane-3-carboxamide (Compound 112)

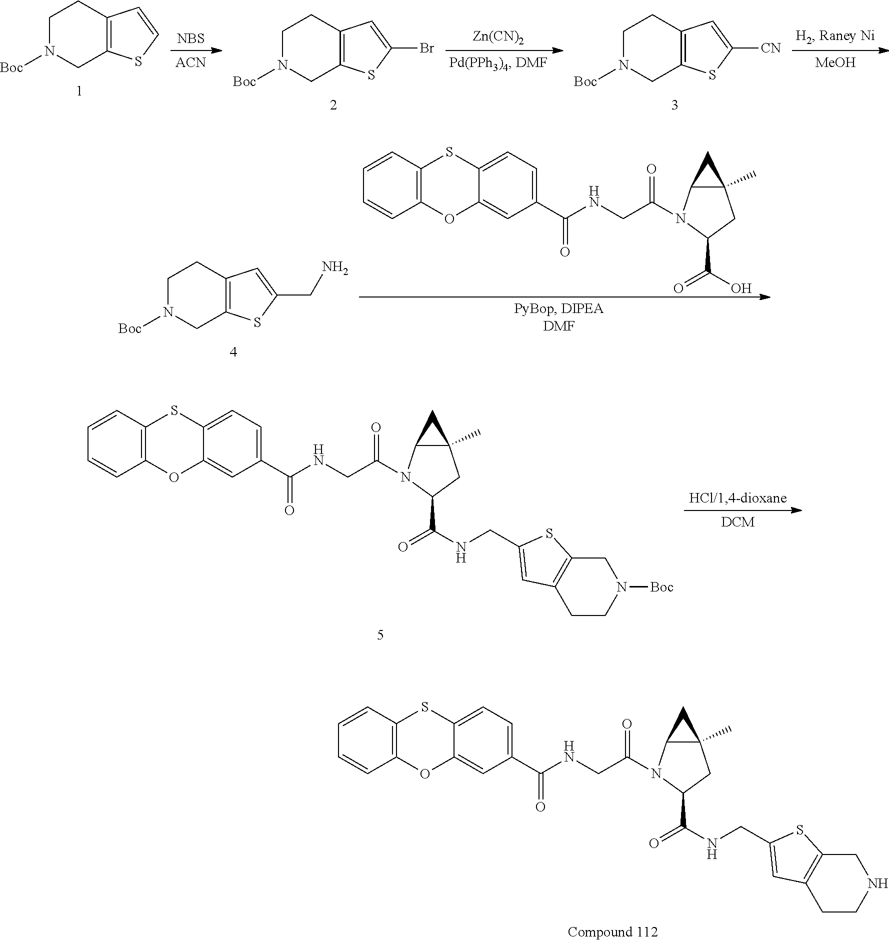

### Step 1: Tert-butyl 2-bromo-4,7-dihydrothieno[2,3-c]pyridine-6(5H)-carboxylate (2)

To a solution of tert-butyl 4,7-dihydrothieno[2,3-c]pyridine-6(5H)-carboxylate (500 mg, 2.10 mmol) in ACN (5 mL) was added NBS (260 mg, 2.21 mmol) under N₂ atmosphere and stirred at 25° C. for 1 hour. The mixture was concentrated under reduced pressure to dryness. The residue was purified by flash chromatography (silica gel, 0-5% EtOAc in PE) to give tert-butyl 2-bromo-4,7-dihydrothieno[2,3-c]pyridine-6(5H)-carboxylate (600 mg, yield 90.5%) as a colorless oil. LC/MS (ESI) m/z: 318/320 (M+H)⁺.

### Step 2: Tert-butyl 2-cyano-4,7-dihydrothieno[2,3-c]pyridine-6(5H)-carboxylate (3)

To a mixture of tert-butyl 2-bromo-4,7-dihydrothieno[2,3-c]pyridine-6(5H)-carboxylate (500 mg, 1.58 mmol) and Zn(CN)₂ (369 mg, 3.16 mmol) in DMF (5 mL) was added Pd(PPh₃)₄ (183 mg, 0.16 mmol) under N₂ atmosphere, the reaction mixture was degassed under N₂ atmosphere for ten times and stirred at 110° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give tert-butyl 2-cyano-4,7-dihydrothieno[2,3-c]pyridine-6(5H)-carboxylate (400 mag, yield 96.2%) as a colorless oil. LC/MS (ESI) n/z. 265 (M+H)⁺.

### Step 3: Tert-butyl 2-(aminomethyl)-4,7-dihydrothieno[2,3-c]pyridine-6(5H)-carboxylate (4)

To a solution of tert-butyl 2-cyano-4,7-dihydrothieno[2,3-c]pyridine-6(5H)-carboxylate (200 mg, 0.76 mmol) in MeOH (5 mL) was added Raney Ni (20 mg, wet), the mixture was degassed under N₂ atmosphere for ten times and stirred under a H₂ balloon at 25° C. overnight. The mixture was filtered, and the filtrate was concentrated under reduced pressure to dryness to give tert-butyl 2-(aminomethyl)-4,7-dihydrothieno[2,3-c]pyridine-6(5H)-carboxylate (150 mg, yield 73.9%) as a colorless oil. LC/MS (ESI) m/z: 269 (M+H)⁺.

### Step 4: Tert-butyl 2-(((1S,3S,5S)-5-methyl-2-((phenoxathiine-3-carbonyl)glycyl)-2-azabicyclo[3.1.0]hexane-3-carboxamido)methyl)-4,7-dihydrothieno[2,3-c]pyridine-6(5H)-carboxylate (5)

To a mixture of (1S,3S,5S)-5-methyl-2-((phenoxathiine-3-carbonyl)glycyl)-2-azabicyclo [3.1.0]hexane-3-carboxylic acid (20 mg, 0.05 mmol) and tert-butyl 2-(aminomethyl)-4,7-dihydrothieno[2,3-c]pyridine-6(5H)-carboxylate (25 mg, 0.10 mmol) in DMF (2 mL) was added DIPEA (35 mg, 0.30 mmol) and PyBOP (24 mg, 0.05 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=20:1) to give tert-butyl 2-(((1S,3S,5S)-5-methyl-2-((phenoxathiine-3-carbonyl)glycyl)-2-azabicyclo[3.1.0]hexane-3-carboxamido)methyl)-4,7-dihydrothieno[2,3-c]pyridine-6(5H)-carboxylate (30 mg, yield 93.8%) as a white solid. LC/MS (ESI) m/z: 675 (M+H)⁺.

### Step 5: (1S,3S,5S)-5-methyl-2-((phenoxathiine-3-carbonyl)glycyl)-N-((4,5,6,7-tetrahydrothieno[2,3-c]pyridin-2-yl)methyl)-2-azabicyclo[3.1.0]hexane-3-carboxamide (Compound 112)

A solution of tert-butyl 2-(((1S,3S,5S)-5-methyl-2-((phenoxathiine-3-carbonyl)glycyl)-2-azabicyclo[3.1.0]hexane-3-carboxamido)methyl)-4,7-dihydrothieno[2,3-c]pyridine-6(5H)-carboxylate (30 mg, 0.04 mmol) in HCl/1,4-dioxane (2 mL, 4M) was stirred under N₂ atmosphere at room temperature for 2 hours. The reaction mixture was concentrated under reduced pressure to dryness. The residue was purified by prep-HPLC to give Compound 112 (6 mg, yield 24.0%) as a white solid. ¹H NMR (400 MHz, MeOD): δ 8.55 (s, 1H), 7.54 (dd, J=8.1, 1.7 Hz, 1H), 7.49 (d, J=1.6 Hz, 1H), 7.24-7.13 (m, 3H), 7.06 (dd, J=16.1, 7.8 Hz, 2H), 6.65 (d, J=15.5 Hz, 1H), 4.80 (dd, J=11.4, 3.2 Hz, 1H), 4.43 (d, J=5.3 Hz, 1H), 4.33 (dd, J=34.5, 16.4 Hz, 2H), 3.79 (d, J=13.9 Hz, 2H), 3.69-3.61 (m, 1H), 3.38 (dd, J=6.0, 2.3 Hz, 1H), 3.07 (t, J=5.2 Hz, 2H), 2.75 (t, J=5.1 Hz, 2H), 2.39 (t, J=12.4 Hz, 1H), 2.15 (dd, J=13.3, 3.3 Hz, 1H), 1.29 (s, 3H), 1.19-1.10 (m, 1H), 0.78 (t, J=5.5 Hz, 1H). LC/MS (ESI) m/z: 575 (M+H)⁺. RT (Method A): 1.49 min.

The following compounds were prepared based on Scheme 36:

| Cmpd # | Reactant A | Reactant B | Characterization Data |
|---|---|---|---|
| 67^{a,f} | 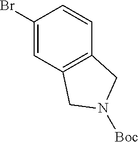 [SMILES: CC(C)(C)OC(=O)N1Cc2ccc(Br)cc2C1] | 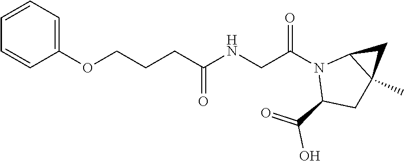 | ¹H NMR (400 MHz, CD₃OD): δ 8.52 (s, 1H), 7.35-7.28 (m, 3H), 7.24 (t, J = 7.8 Hz, 2H), 6.92-6.88 (m, 3H), 4.80 (dd, J = 11.4, 3.1 Hz, 1H), 4.54 (d, J = 5.9 Hz, 4H), 4.36 (d, J = 20.0 Hz, 2H), 4.23 (d, J = 16.6 Hz, 1H), 4.12 (d, J = 16.6 Hz, 1H), 4.00 (t, J = 6.2 Hz, 2H), 3.35 (s, 1H), 2.47 (t, J = 7.4 Hz, 2H), 2.38 (t, J = 12.4 Hz, 1H), 2.13 (dd, J = 13.4, 3.2 Hz, 1H), 2.06 (dd, J = 13.6, 6.8 Hz, 2H), 1.28 (s, 3H), 1.18-1.09 (m, 1H), 0.79 (t, J = 5.7 Hz, 1H). LC/MS (ESI) (m/z): 491 (M + H)⁺. RT (Method A): 1.09 min. |
| 170^{a} | 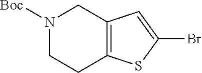 [SMILES: CC(C)(C)OC(=O)N1CCc2sc(Br)cc2C1] | 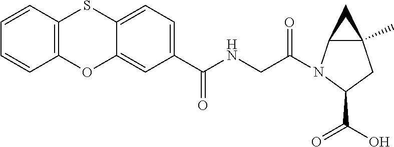 | ¹H NMR (400 MHz, CD₃OD): δ 8.50 (s, 1H), 7.56 (dd, J = 8.1, 1.9 Hz, 1H), 7.51 (d, J = 1.8 Hz, 1H), 7.25 (d, J = 8.1 Hz, 1H), 7.18 (ddd, J = 9.3, 7.0, 1.1 Hz, 2H), 7.10-7.04 (m, 2H), 6.73 (s, 1H), 4.80 (d, J = 8.0 Hz, 1H), 4.46 (s, 2H), 4.33 (d, J = 3.1 Hz, 2H), 4.10 (d, J = 4.8 Hz, 2H), 3.44-3.39 (m, 3H), 3.02-2.99 (m, 2H), 2.43-2.36 (m, 1H), 2.15 (dd, J = 13.3, 3.4 Hz, 1H), 1.29 (s, 3H), 1.14 (dd, J = 5.8, 2.4 Hz, 1H), 0.79 (td, J = 5.9, 1.3 Hz, 1H). LC/MS (ESI) m/z: 575 (M + H)⁺. RT (Method A): 1.51 min. |
| 210^{b,c} | 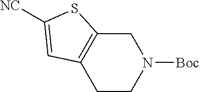 [SMILES: CC(C)(C)OC(=O)N1CCc2cc(C#N)sc2C1] | 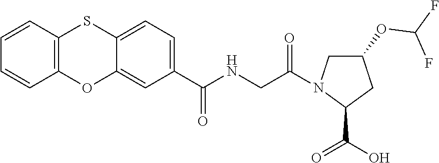 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 7.56-7.53 (m, 1H), 7.49 (d, J = 1.8 Hz, 1H), 7.22 (d, J = 8.1 Hz, 1H), 7.21-7.17 (m, 1H), 7.15 (dd, J = 7.7, 1.5 Hz, 1H), 7.10- 7.06 (m, 1H), 7.04 (d, J = 8.3 Hz, 1H), 6.54 (dd, J = 101.7, 47.1 Hz, 2H), 4.99 (s, 1H), 4.57-4.39 (m, 3H), 4.24-4.10 (m, 2H), 3.98-3.89 (m, 1H), 3.85 (s, 1H), 3.75 (s, 2H), 3.06 (t, J = 5.9 Hz, 2H), 2.74 (d, J = 5.8 Hz, 2H), 2.47-2.30 (m, 1H), 2.24-2.17 (m, 1H). LC/MS (ESI) m/z: 615 (M + H)⁺. RT (Method A): 1.00 min. |
| 224^{d,e} | 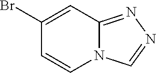 | 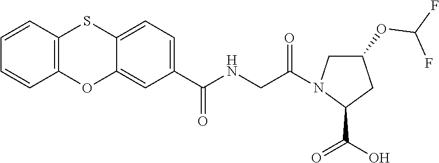 [SMILES: O=C(NCC(=O)N1C[C@H](OC(F)F)C[C@H]1C(=O)O)c1ccc2c(c1)Oc1ccccc1S2] | ¹H NMR (400 MHz, DMSO-d6): δ 9.18 (d, J = 17.3 Hz, 1H), 9.00-8.78 (m, 1H), 8.75-8.61 (m, 1H), 8.51-8.43 (m, 1H), 7.61 (d, J = 7.9 Hz, 1H), 7.56 (s, 2H), 7.37 (t, J = 6.8 Hz, 1H), 7.30-7.23 (m, 2H), 7.16-7.11 (m, 2H), 7.02- 6.77 (m, 2H), 4.91 (d, J = 34.6 Hz, 1H), 4.50-4.38 (m, 2H), 4.35-4.32 (m, 1H), 4.22-4.17 (m, 1H), 4.02-3.98 (m, 1H), 3.90-3.86 (m, 1H), 3.81-3.79 (m, 1H), 2.16-2.10 (m, 1H), 2.05-1.95 (m, 1H). LC/MS (ESI) m/z: 595 (M + H)⁺. RT (Method A): 1.70 min. |
| 225^{d,e} | 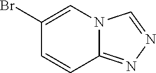 | 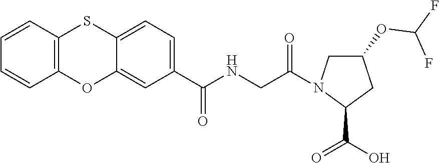 [SMILES: O=C(NCC(=O)N1C[C@H](OC(F)F)C[C@H]1C(=O)O)c1ccc2c(c1)Oc1ccccc1S2] | ¹H NMR (400 MHz, DMSO-d6): δ 9.18 (d, J = 55.1 Hz, 1H), 8.98-8.79 (m, 1H), 8.74-8.61 (m, 1H), 8.45 (d, J = 29.4 Hz, 1H), 7.73 (dd, J = 13.0, 9.4 Hz, 1H), 7.63-7.56 (m, 2H), 7.40-7.37 (m, 1H), 7.31-7.24 (m, 3H), 7.15 (dd, J = 8.9, 1.7 Hz, 2H), 7.00-6.62 (m, 1H), 4.98-4.85 (m, 1H), 4.44-4.37 (m, 1H), 4.36-4.24 (m, 2H), 4.22-4.11 (m, 1H), 4.08-4.00 (m, 1H), 3.92-3.83 (m, 1H), 3.82-3.77 (m, 1H), 2.37-2.29 (m, 1H), 2.17-2.09 (m, 1H). LC/MS (ESI) m/z: 595 (M + H)⁺. RT (Method A): 1.71 min. |
| 228^{a,e} | 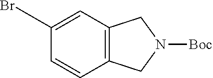 [SMILES: CC(C)(C)OC(=O)N1Cc2ccc(Br)cc2C1] | 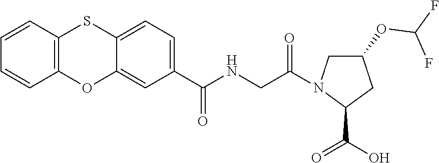 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 8.11 (d, J = 15.7 Hz, 1H), 7.60-7.52 (m, 2H), 7.51-7.46 (m, 2H), 7.20 (dt, J = 8.0, 2.2 Hz, 3H), 7.15 (dd, J = 7.8, 1.6 Hz, 1H), 7.08 (dd, J = 7.5, 1.3 Hz, 1H), 7.03 (dd, J = 8.1, 1.1 Hz, 1H), 6.48 (dd, J = 74.6, 57.0 Hz, 1H), 5.01 (s, 1H), 4.61 (t, J = 7.8 Hz, 1H), 4.53 (s, 2H), 4.26 (d, J = 16.7 Hz, 1H), 4.14 (d, J = 16.7 Hz, 1H), 3.96 (dd, J = 11.5, 4.6 Hz, 1H), 3.86 (d, J = 12.0 Hz, 1H), 2.47 (ddd, J = 12.2, 8.3, 3.8 Hz, 1H), 2.28-2.20 (m, 1H). LC/MS (ESI) (m/z): 594 (M + H)⁺. RT (Method A): 1.54 min. |
| 245^{d,e} | 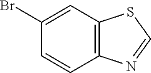 | 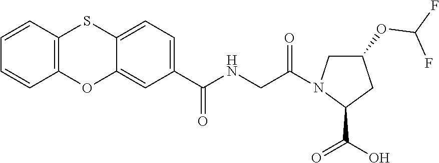 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 9.19-9.05 (m, 1H), 8.07-7.93 (m, 2H), 7.56- 7.41 (m, 3H), 7.21-7.11 (m, 3H), 7.08-6.99 (m, 2H), 6.72-6.32 (m, 1H), 5.04- 4.91 (m, 1H), 4.64-4.53 (m, 3H), 4.25-4.11 (m, 2H), 4.03-3.94 (m, 1H), 3.91-3.83 (m, 1H), 2.54-2.42 (m, 1H), 2.30-2.18 (m, 1H). LC/MS (ESI) m/z: 611 (M + H)⁺. RT (Method A): 2.15 min. |

^{a}Steps 2-5 only.
^{b}Steps 3-5 only.
^{c}Hydrogenation of nitrile was performed in the presence of NH_{3•}H₂O.
^{d}Steps 2-4 only.
^{e}HATU was used in place of PyBOP in Step 4.
^{f}TFA was used in place of HCl/1,4-dioxane in Step 5.

### Scheme 37. Synthesis of (1S,3S,5S)-N-((R)-1-(1H-pyrrolo[3,2-c]pyridin-2-yl)ethyl)-2-(2-(5-(2,4-difluorophenyl)-1-oxoisoindolin-2-yl)acetyl)-5-methyl-2-azabicyclo[3.1.0]hexane-3-carboxamide (Compound 113)

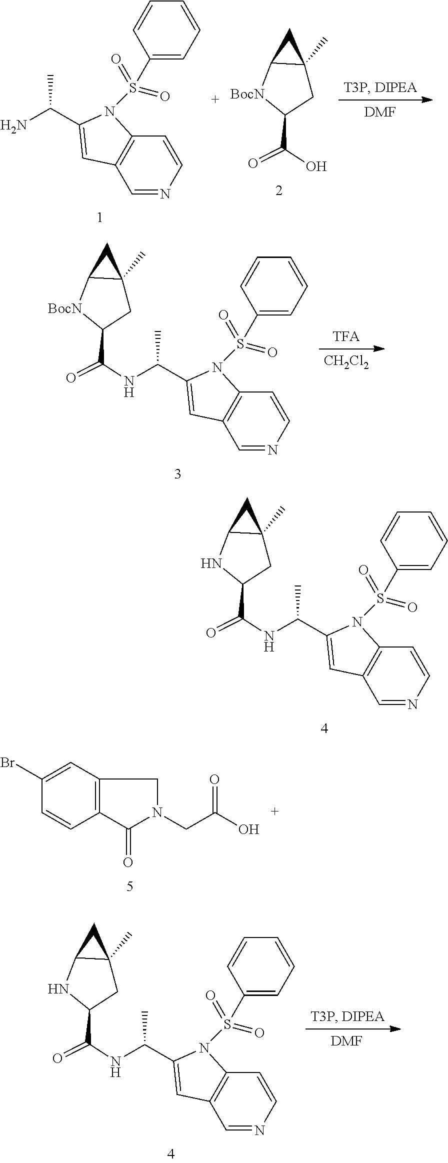

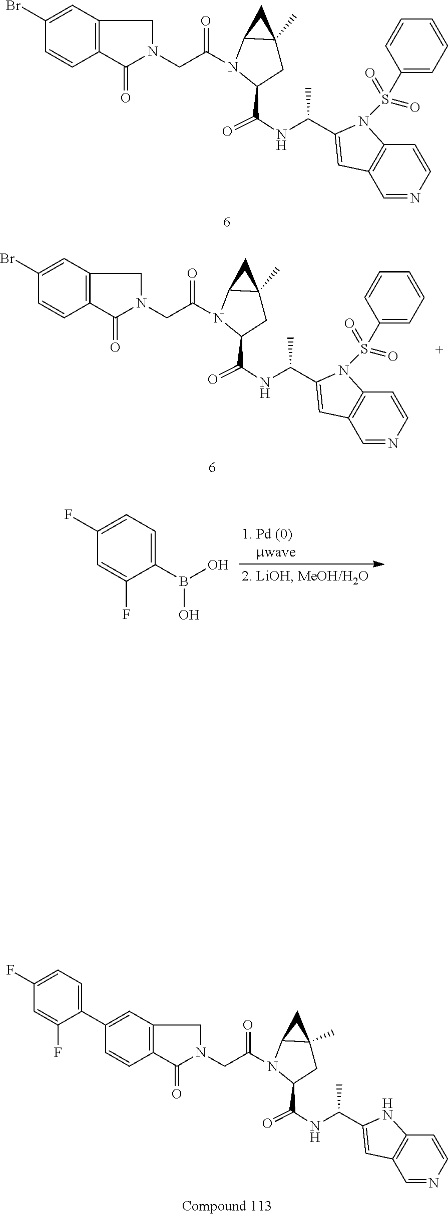 [SMILES: C[C@@H](NC(=O)[C@@H]1C[C@]2(C)C[C@@H]2N1C(=O)CN1Cc2cc(Br)ccc2C1=O)c1cc2cnccc2n1S(=O)(=O)c1ccccc1 ; C[C@@H](NC(=O)[C@@H]1C[C@]2(C)C[C@@H]2N1C(=O)CN1Cc2cc(Br)ccc2C1=O)c1cc2cnccc2n1S(=O)(=O)c1ccccc1 ; OB(O)c1ccc(F)cc1F ; C[C@@H](NC(=O)[C@@H]1C[C@]2(C)C[C@@H]2N1C(=O)CN1Cc2cc(-c3ccc(F)cc3F)ccc2C1=O)c1cc2cnccc2[nH]1]

### Step 1: tert-butyl (1S,3S,5S)-5-methyl-3-(((R)-1-(1H-(phenylsulfonyl)-1H-pyrrolo[3,2-c]pyridin-2-yl)ethyl)carbamoyl)-2-azabicyclo[3.1.0]hexane-2-carboxylate (3)

To a solution of (1S,3S,5S)-2-tert-butoxycarbonyl-5-methyl-2-azabicyclo[3.1.0]hexane-3-carboxylic acid (45.00 mg, 0.1865 mmol, 1.0 equiv.) in DMF (3 mL) was added (1R)-1-[1-(benzenesulfonyl)pyrrolo[3,2-c]pyridin-2-yl]ethanamine;dihydrochloride (76.79 mg, 0.2052 mmol, 1.2 equiv.), T3P (50 mass %) in ethyl acetate (0.168 mL, 0.281 mmol, 1.5 equiv.), and DIPEA (97.6 μL, 0.560 mmol, 3.0 equiv.). The reaction mixture was stirred at room temperature for 30 minutes and then purified by combi Flash. 4 g column, solvent A=CH₂Cl₂, solvent B=MeOH, 100% A to 5% B to give tert-butyl (1S,3S,5S)-3-[[(1R)-1-[1-(benzenesulfonyl)pyrrolo[3,2-c]pyridin-2-yl]ethyl]carbamoyl]-5-methyl-2-azabicyclo[3.1.0]hexane-2-carboxylate (80.00 mg, 0.1525 mmol, 81.77% yield). LC/MS (ESI) m/z: 525 (M+H)⁺.

### Step 2: Synthesis of (1S,3S,5S)-5-methyl-1-((R)-1-(1H-(phenylsulfonyl)-H-pyrrolo[3,2-c]pyridin-2-yl)ethyl)-2-azabicyclo [3.1.0]hexane-3-carboxamide (4)

TFA (0.43 mL) was added to a solution of tert-butyl (1S,3S,5S)-3-[[(1R)-1-[1-(benzenesulfonyl)pyrrolo[3,2-c]pyridin-2-yl]ethyl]carbamoyl]-5-methyl-2-azabicyclo[3.1.0]hexane-2-carboxylate (0.1000 g, 0.1906 mmol, 1.0 equiv.) in CH₂Cl₂ (1 mL) at ice-bath temperature. The reaction mixture was stirred at room temperature for 4 hours and then concentrated to dryness to give (1S,3S,5S)-N-[(1R)-1-[1-(benzenesulfonyl)pyrrolo[3,2-c]pyridin-2-yl]ethyl]-5-methyl-2-azabicyclo[3.1.0]hexane-3-carboxamide (0.07567 g, 0.1783 mmol, 93.51% yield) as a white solid which was used in the next step without further purification. LC/MS (ESI) m/z: 425 (M+H)⁺.

### Step 3: (1S,3S,5S)-2-(2-(5-bromo-1-oxoisoindolin-2-yl)acetyl)-5-methyl-N-((R)-1-(1-(phenylsulfonyl)-1H-pyrrolo[3,2-c]pyridin-2-yl)ethyl)-2-azabicyclo[3.1.0]hexane-3-carboxamide (6)

To a solution of 2-(5-bromo-1-oxo-isoindolin-2-yl)acetic acid (45.81 mg, 0.1696 mmol, 1.2 equiv.) in DMF (5 mL) was added (1S,3S,5S)-N-[(1R)-1-[1-(benzenesulfonyl)pyrrolo[3,2-c]pyridin-2-yl]ethyl]-5-methyl-2-azabicyclo[3.1.0]hexane-3-carboxamide (B, 60.00 mg, 0.1413 mmol 1.0 equiv.), T3P (50 mass %) in ethyl acetate (0.1270 mL, 0.212 mmol, 1.5 equiv.), and DIPEA (0.0740 mL, 0.424 mmol, 3.0 equiv.). The reaction mixture was stirred at room temperature for 30 minutes and then diluted with water (5 mL) and stirred for 30 minutes. The filtrate was decanted, and the residue was washed with water, dried over Na₂SO₄, and then concentrated to obtain a brown oil which was purified by combi Flash chromatography, 12 g column, solvent A=CH₂Cl₂, solvent B=MeOH. 100% A to 5% B to give (1S,3S,5S)-N-[(LR)-1-[1-(benzenesulfonyl)pyrrolo[3,2-c]pyridin-2-yl]ethyl]-2-[2-(5-bromo-1-oxo-isoindolin-2-yl)acetyl]-5-methyl-2-azabicyclo[3.1.0]hexane-3-carboxamide (73.55 mug, 0.1087 mmol, 100 mass %, 76.91% yield). LC/MS (ESI) m/z: 677 (M+H)⁺.

### Step 4: (1S,3S,5S)-N-((R)-1-(1H-pyrrolo[3,2-c]pyridin-2-yl)ethyl)-2-(2-(5-(2,4-difluorophenyl)-1-oxoisoindolin-2-yl)acetyl)-5-methyl-2-azabicyclo[3.1.0]hexane-3-carboxamide (Compound 113)

A mixture of (2,4-difluorophenyl)boronic acid (4.201 mg, 0.02660 mmol, 1.2 equiv.), (1S,3S,5S)-N-[(1R)-1-[1-(benzenesulfonyl)pyrrolo[3,2-c]pyridin-2-yl]ethyl]-2-[2-(5-bromo-1-oxo-isoindolin-2-yl)acetyl]-5-methyl-2-azabicyclo[3.1.0]hexane-3-carboxamide (15.00 mg, 0.02217 mmol, 1.0 equiv.), Pd(PPh₃)₂dppf-CH₂Cl₂ (1.829 mg, 0.002217 mmol, 0.1 equiv.), Na₂CO₃ (4.699 mg, 0.04434 mmol, 2 equiv.) in 1,4-dioxane (3 mL) and water (0.3 mL) was stirred in a microwave reactor at 110° C. for 30 min. The reaction mixture was filtered through Celite pad. The cake was washed with water and EtOAc. The two layers were separated. aqueous layer was extracted with EtOAc, washed with brine, dried over Na₂SO₄, and concentrated to give a brown oil. To a solution of the brown oil in MeOH (1.5 mL) and water (0.5 mL) was added LiOH (1.000 mg, 0.04176 mmol, 2 equiv.). The reaction mixture was stirred at room temperature overnight and then concentrated to remove MeOH. The pH was adjusted to 1, and then the mixture was purified directly using prep HPLC to give Compound 113 (1.890 mg, 0.003318 mmol, 14.97% yield). LC/MS (ESI) m/z:570 (M+H)⁺. ¹H NMR (400 MHz, DMSO-d6): δ 10.96 (s, 1H), 8.59-8.45 (m, 1H), 8.44 (s. 1H), 7.77 (d, J=8.6 Hz, 2H), 7.68 (s, 1H), 7.65-7.51 (m, 1H), 7.36 (t, J=10.4 Hz, 1H₁), 7.26-7.11 (m, 1H), 6.97 (s, 2H), 6.69 (d, J=5.7 Hz, 1H), 6.30 (s, 1H), 6.18 (s, 1H). 5.01 (t, J=7.5 Hz, 1H), 4.66 (d, J=16.5 Hz, 1H), 4.58 (d, J=17.6 Hz, 1H), 4.42 (d, J=17.0 Hz, 2H), 3.39 (d, J=6.4 Hz, 1H), 2.26 (t, J=12.3 Hz, 1H), 2.05-1.87 (m, 1H), 1.38 (d, J=7.0 Hz, 3H). 1.32 (d, J=4.5 Hz, 1H), 1.19 (d, J=12.5 Hz, 2H), 0.78 (d, J=7.9 Hz, 1H). RT (Method A): 1.60 min.

### Scheme 38. Synthesis of (2S,4R)-N-((1H-pyrrolo[3,2-c]pyridin-2-yl)methyl)-4-(difluoromethoxy)-1-((3-phenoxybenzoyl)glycyl)pyrrolidine-2-carboxamide (Compound 127)

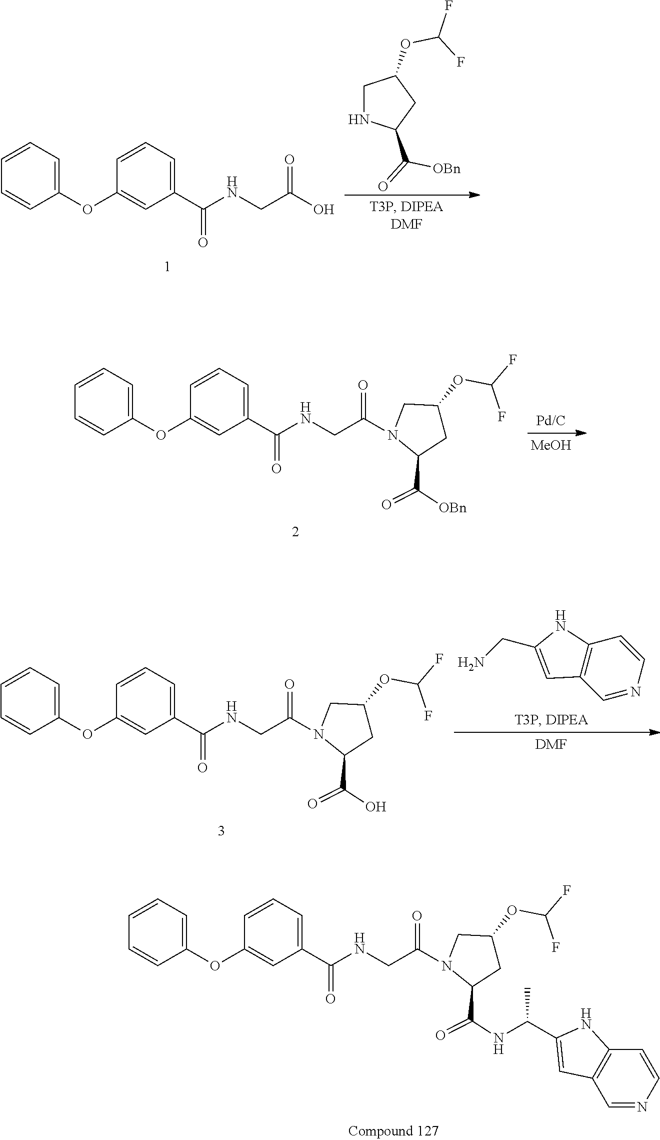

### Step 1: benzyl (2S,4R)-4-(difluoromethoxy)-1-((3-phenoxybenzoyl)glycyl)pyrrolidine-2-carboxylate (2)

To a solution of 2-[(3-phenoxybenzoyl)amino]acetic acid (50.00 mg, 0.1843 mmol, 1.0 equiv.) in DMF (2 mL) was added benzyl (2S,4R)-4-(difluoromethoxy)pyrrolidine-2-carboxylate hydrochloride (68.05 mg, 0.2212 mmol, 1.2 equiv.), T3P (0.166 mL, 0.277 mmol, 1.5 equiv., 50 mass % in ethyl acetate), and DIPEA (96.40 mL, 553 mmol, 3.0 equiv.). The reaction mixture was stirred at room temperature for 30 minutes. Water (5 mL) was added to the reaction mixture, and the solid was collected by filtration, rinsed with water, and dried to give benzyl (2S,4R)-4-(difluoromethoxy)-1-[2-[(3-phenoxybenzoyl)amino]acetyl]pyrrolidine-2-carboxylate (85.00 mg, 0.1621 mmol, 87.93% yield) as a brown solid. LC/MS (ESI) m/z: 525 (M+H)⁺.

### Step 2: (2S,4R)-4-(difluoromethoxy)-1-((3-phenoxybenzoyl)glycyl)pyrrolidine-2-carboxylic acid (3)

To a solution of benzyl (2S,4R)-4-(difluoromethoxy)-1-[2-[(3-phenoxybenzoyl)amino]acetyl]pyrrolidine-2-carboxylate (40.00 mg, 0.07626 mmol, 1.0 equiv.) in MeOH (2 mL) was added 5% Pd/C (16.23 mg, 0.007625 mmol, 0.1 equiv.). The flask was evacuated and then backfilled with H₂ gas in a balloon. The reaction mixture was stirred at room temperature overnight and then filtered through Celite pad. The filtrate was concentrated and dried in vacuo to give (2S,4R)-4-(difluoromethoxy)-1-[2-[(3-phenoxybenzoyl)amino]acetyl]pyrrolidine-2-carboxylic acid (30.00 mg, 0.06906 mmol, 90.56% yield) as a clear oil, which was used without further purification. LC/MS (ESI) m/z: 435 (M+H)⁺.

### Step 3: (2S,4R)-N-((1H-pyrrolo[3,2-c]pyridin-2-yl)methyl)-4-(difluoromethoxy)-1-((3-phenoxybenzoyl)glycyl)pyrrolidine-2-carboxamide (Compound 127)

To a solution of (2S,4R)-4-(difluoromethoxy)-1-[2-[(3-phenoxybenzoyl)amino]acetyl]pyrrolidine-2-carboxylic acid (14.00 mg, 0.03223 mmol, 1.0 equiv.) in DMF (2 mL) was added 1H-pyrrolo[3,2-c]pyridin-2-ylmethanamine dihydrochloride (8.512 mg, 0.03867 mmol, 1.2 equiv.), T3P (50 mass %) in ethyl acetate (28.9 μL, 0.0483 mmol, 1.5 equiv.), and DIPEA (16.90 μL, 0.0969 mmol, 3.0 equiv.). The reaction mixture was stirred at room temperature for 30 minutes and then purified directly by prep HPLC to obtain Compound 127 (3.129 mg, 0.005552 mmol, 12.85% yield). LC/MS (ESI) m/z: 564 (M+H)⁺. ¹H NMR (400 MHz, DMSO-d6): δ 11.15 (s, 1H), 8.86 (s, 1H), 8.70 (s, 1H), 8.59 (d, J=9.9 Hz, 2H), 8.19 (s, 1H), 7.96 (d, J=5.6 Hz, 1H), 7.60 (t, J=8.7 Hz, 2H), 7.49-7.39 (m, 1H), 7.39-7.30 (m, 3H), 7.20-7.06 (m, 2H), 6.98 (t, J=7.5 Hz, 1H), 6.74 (s, 1H), 6.31 (s, 1H), 4.90 (d, J=4.9 Hz, 1H), 4.62-4.22 (m, 2H), 4.12 (dd, J=16.6, 5.8 Hz, 1H), 3.95 (dd, J=16.7, 5.6 Hz, 1H), 3.81 (dd, J=11.6, 4.5 Hz, 1H), 2.26 (td, J=9.9, 4.9 Hz, 1H), 2.17-1.96 (m, 1H). RT (Method A): 1.43 min.

The following compounds were prepared based on Steps 1 and 2 in Scheme 38:

| Cmpd | | | |
|---|---|---|---|
| # | Reactant A | Reactant B | Characterization Data |
| 118 | 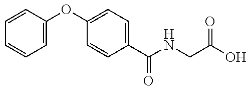 [SMILES: O=C(O)CNC(=O)c1ccc(Oc2ccccc2)cc1] | 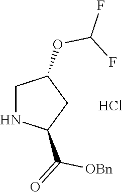 [SMILES: O=C(OCc1ccccc1)[C@@H]1C[C@@H](OC(F)F)CN1 ; Cl] | LC/MS (ESI) m/z: 435 (M + H)⁺. RT (Method A): 1.85 min. |
| 119 | 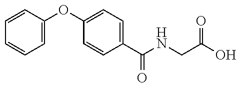 [SMILES: O=C(O)CNC(=O)c1ccc(Oc2ccccc2)cc1] | 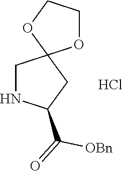 [SMILES: O=C(OCc1ccccc1)[C@@H]1CC2(CN1)OCCO2 ; Cl] | LC/MS (ESI) m/z: 427 (M + H)⁺. RT (Method A): 1.64 min. |

### Scheme 39. Synthesis of (2S,4R)-1-((9,9-difluoro-9H-fluorene-3-carbonyl)glycyl)-4-(difluoromethoxy)-N-((R)-2-hydroxy-1-(1H-pyrrolo[3,2-c]pyridin-2-yl)ethyl) pyrrolidine-2-carboxamide (Compound 128)

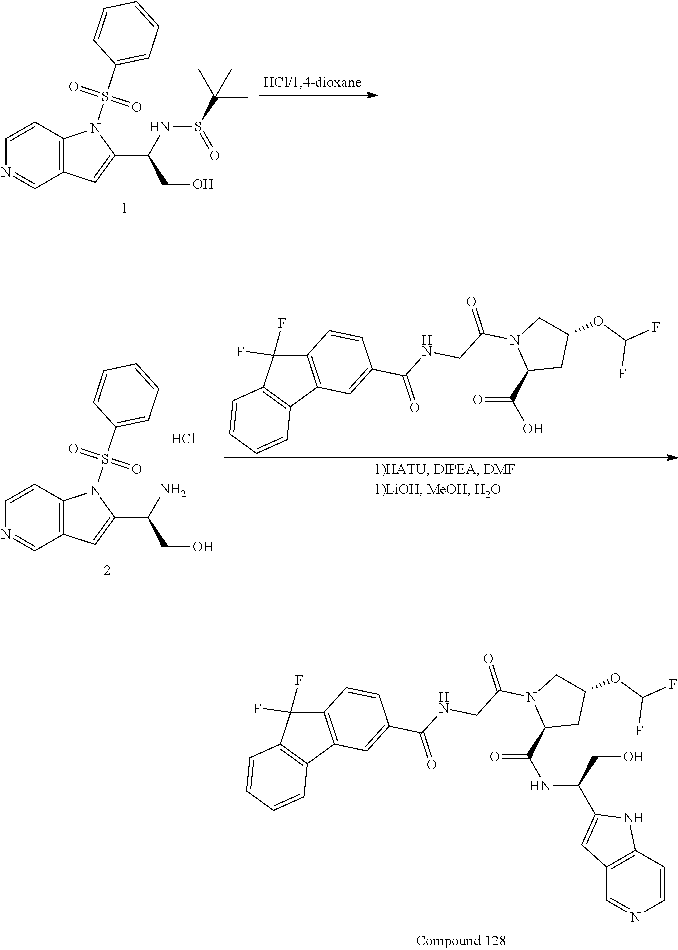 [SMILES: CC(C)(C)[S@@](=O)N[C@@H](CO)c1cc2cnccc2n1S(=O)(=O)c1ccccc1 ; N[C@@H](CO)c1cc2cnccc2n1S(=O)(=O)c1ccccc1 ; Cl ; O=C(NCC(=O)N1C[C@H](OC(F)F)C[C@H]1C(=O)O)c1ccc2c(c1)-c1ccccc1C2(F)F ; O=C(NCC(=O)N1C[C@H](OC(F)F)C[C@H]1C(=O)N[C@@H](CO)c1cc2cnccc2[nH]1)c1ccc2c(c1)-c1ccccc1C2(F)F]

### Step 1: (R)-2-amino-2-(1-(phenylsulfonyl)-H-pyrrolo[3,2-c]pyridin-2-yl)ethan-1-ol hydrochloride (2)

A solution of (R)-N-((R)-2-hydroxy-1-(1-(phenylsulfonyl)-1H-pyrrolo[3,2-c]pyridin-2-yl)ethyl)-2-methylpropane-2-sulfinamide (200 mg, 0.47 mmol) in HCl/1,4-dioxane (2 mL, 4M) was stirred under N₂ atmosphere at room temperature for 2 hours. The reaction mixture was concentrated to dryness under reduced pressure to give (R)-2-amino-2-(1-(phenylsulfonyl)-1H-pyrrolo[3,2-c]pyridin-2-yl)ethan-1-ol hydrochloride (160 mg, yield 95.8%) as a white solid, which was used directly in the next step without further purification. LC/MS (ESI) n/z: 318 (M+H)⁺.

### Step 2: (2R,4R)-1-((9,9-difluoro-9H-fluorene-3-carbonyl)glycyl)-4-(difluoromethoxy)-N-((S)-2-hydroxy-1-(1H-pyrrolo[3,2-c]pyridin-2-yl)ethyl)pyrrolidine-2-carboxamide (Compound 128)

To a mixture of (R)-2-amino-2-(1-(phenylsulfonyl)-1H-pyrrolo[3,2-c]pyridin-2-yl)ethan-1-ol hydrochloride (23 mg, 0.067 mmol) and (2S,4R)-1-((9,9-difluoro-9H-fluorene-3-carbonyl)glycyl)-4-(difluoromethoxy)pyrrolidine-2-carboxylic acid (30 mg, 0.067 mmol) in DMF (1 mL) was added DIPEA (26 mg, 0.20 mmol) and HATU (38 mg, 0.10 mmol) under N₂ atmosphere and the reaction mixture was stirred at room temperature for 1 hours. To the mixture was added LiOH (4.8 mg, 0.20 mmol), MeOH (0.5 mL) and water (0.1 mL) and the mixture was stirred at room temperature for 6 hours. The mixture was diluted with EtOAc and washed with saturated aq. NaHCO₃ solution and brine, dried over anhydrous Na₂SO₄, filtered, and concentrated under reduced pressure to dryness. The residue was purified by prep-TLC (DCM:MeOH=7:1) and further purified by prep-HPLC to give Compound 128 (1.04 mg, yield 2.5%) as a white solid. ¹H NMR (400 MHz, CD₃OD): δ 8.82 (s, 1H), 8.45 (s, 1H), 8.21 (s, 1H), 7.92 (d, J=6.9 Hz, 2H), 7.76-7.70 (m, 2H), 7.67 (d, J=7.4 Hz, 1H), 7.56 (t, J=7.2 Hz, 1H), 7.44 (dd, J=12.1, 6.2 Hz, 2H), 6.84 (s, 1H), 6.55 (t, J=74.8 Hz, 1H), 5.30 (t, J=5.9 Hz, 1H), 5.08 (s, 1H), 4.69 (t, J=8.1 Hz, 1H), 4.37 (d, J=16.9 Hz, 1H), 4.22 (d, J=16.6 Hz, 1H), 4.01 (dd, J=10.1, 4.9 Hz, 2H), 3.93 (dd, J=11.3, 4.9 Hz, 2H), 2.52 (dd, J=14.7, 8.5 Hz, 1H), 2.34 (dd, J=12.5, 6.2 Hz, 1H). LC/MS (ESI) m/z: 626 (M+H)⁺. RT (Method A): 1.37 min.

### Scheme 40. Synthesis of (S)-N-((R)-1-(1H-pyrrolo[3,2-c]pyridin-2-yl)ethyl)-2-((phenoxathiine-3-carbonyl)glycyl)-2-azaspiro[4.4]nonane-3-carboxamide (Compound 131)

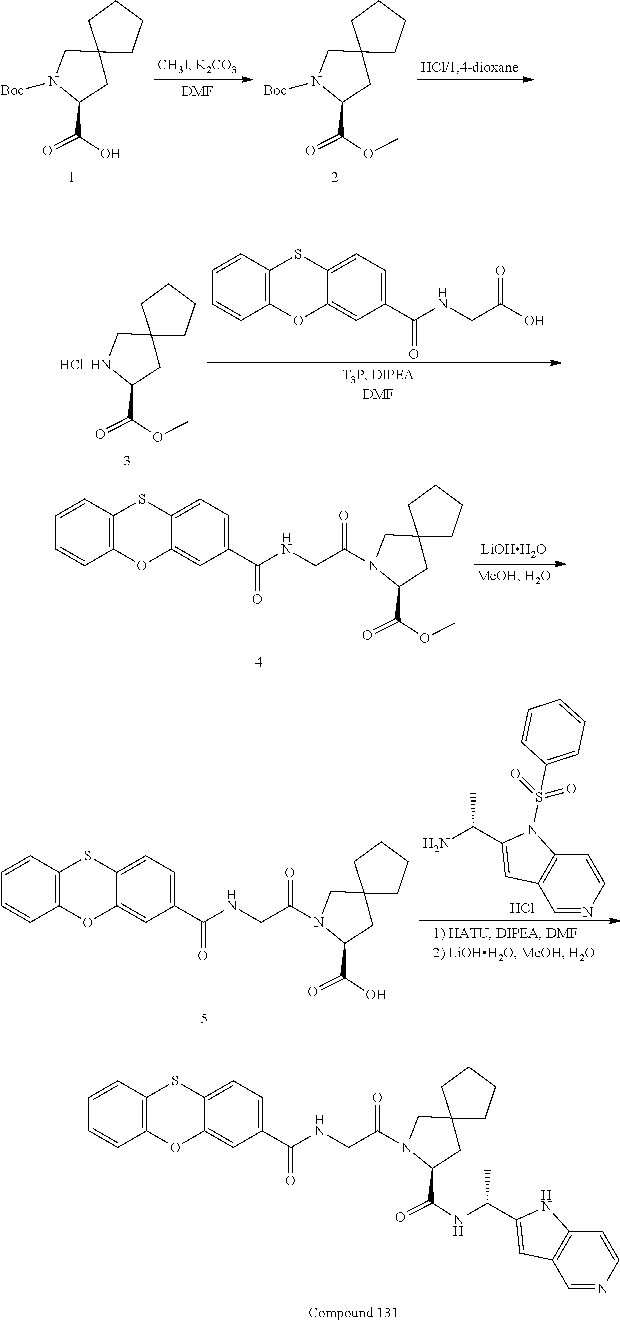

### Step 1: 2-(Tert-butyl) 3-methyl (S)-2-azaspiro[4.4]nonane-2,3-dicarboxylate (2)

To a mixture of (S)-2-(tert-butoxycarbonyl)-2-azaspiro[4.4]nonane-3-carboxylic acid (100 mg, 0.37 mmol) and K₂CO₃ (154 mg, 1.11 mmol) in DMF (3 mL) was added CH₃I (39 mg, 0.56 mmol) at 0° C. and the mixture was stirred at 25° C. for 16 hours. The mixture was diluted with EtOAc and washed with saturated aq. NH₄Cl solution twice, dried over anhydrous Na₂SO₄, filtered, and concentrated under reduced pressure to dryness to give 2-(tert-butyl) 3-methyl (S)-2-azaspiro[4.4]nonane-2,3-dicarboxylate (88 mg, yield 83.8%) as a brown oil. LC/MS (ESI) m/z: 284 (M+H)⁺.

### Step 2: Methyl (S)-2-azaspiro[4.4]nonane-3-carboxylate hydrochloride (3)

A solution of 2-(tert-butyl) 3-methyl (S)-2-azaspiro[4.4]nonane-2,3-dicarboxylate (88 mg, 0.31 mmol) in HCl/1,4-dioxane (3 mL, 4M) was stirred under N₂ atmosphere at room temperature for 2 hours. The reaction mixture was concentrated to dryness under reduced pressure to give methyl (S)-2-azaspiro[4.4]nonane-3-carboxylate hydrochloride (50 mg, yield 89.3%) as a brown solid, which was used directly in the next step without further purification. LC/MS (ESI) m/z: 184 (M+H)⁺.

### Step 3: Methyl (S)-2-((phenoxathiine-3-carbonyl)glycyl)-2-azaspiro[4.4]nonane-3-carboxylate (4)

To a mixture of (phenoxathiine-3-carbonyl)glycine (50 mg, 0.17 mmol) and methyl (S)-2-azaspiro[4.4]nonane-3-carboxylate hydrochloride (46 mg, 0.26 mmol) in DMF (3 mL) was added DIPEA (132 mg, 1.02 mmol) and T3P (324 mg, 0.51 mmol, 50% wt. in EtOAc) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-50% EtOAc in PE) to give methyl (S)-2-((phenoxathiine-3-carbonyl)glycyl)-2-azaspiro[4.4]nonane-3-carboxylate (71 mg, yield 92.2%) as a white solid. LC/MS (ESI) m/z: 467 (M+H)⁺.

### Step 4: (S)-2-((phenoxathiine-3-carbonyl)glycyl)-2-azaspiro[4.4]nonane-3-carboxylic acid (5)

To a solution of methyl (S)-2-((phenoxathiine-3-carbonyl)glycyl)-2-azaspiro[4.4]nonane-3-carboxylate (71 mg, 0.15 mmol) in MeOH (3 mL) and H₂O (1 mL) was added LiOH·H₂O (6 mg, 0.30 mmol) and the reaction mixture was stirred at room temperature for 2 hours. The mixture was acidified with IN aq. HCl to pH˜3 and extracted with EtOAc twice. The combined organic layers were dried over anhydrous Na₂SO₄, filtered, and concentrated under reduced pressure to dryness to give (S)-2-((phenoxathiine-3-carbonyl)glycyl)-2-azaspiro[4.4]nonane-3-carboxylic acid (60 mg, yield 87.0%) as a brown solid, which was used directly in the next step without further purification. LC/MS (ESI) m/z: 453 (M+H)⁺.

### Step 5: (S)-N-((R)-1-(1H-pyrrolo[3,2-c]pyridin-2-yl)ethyl)-2-((phenoxathiine-3-carbonyl)glycyl)-2-azaspiro[4.4]nonane-3-carboxamide (Compound 131)

To a mixture of (S)-2-((phenoxathiine-3-carbonyl)glycyl)-2-azaspiro[4.4]nonane-3-carboxylic acid (30 mg, 0.07 mmol) and (R)-1-(1-(phenylsulfonyl)-1H-pyrrolo[3,2-c]pyridin-2-yl)ethan-1-amine hydrochloride (30.0 mg, 0.11 mmol) in DMF (3 mL) was added DIPEA (54 mg, 0.42 mmol) and HATU (40.0 mg, 0.1 mmol) under N₂ atmosphere and the reaction mixture was stirred at room temperature for 2 hours. To the mixture the solution of LiOH·H₂O (4.4 mg, 0.11 mmol) in MeOH (2 mL) and water (1 mL). The mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=10:1) and further purified by prep-HPLC to give Compound 131 (2 mg, yield 5.0%) as a white solid. ¹H NMR (400 MHz, CD₃OD): δ 8.65 (s, 1H), 7.90 (d, J=5.6 Hz, 1H), 7.65-7.49 (m, 21H), 7.28 (d, J=8.1 Hz, 11H), 7.22-7.14 (m, 2H), 7.13-7.02 (m, 3H), 6.52 (s, 1H), 5.34-5.29 (m, 1H), 4.58 (s, 2H), 4.41 (d, J=8.9 Hz, 1H), 3.64 (d, J=9.7 Hz, 1H), 3.53 (d, J=9.9 Hz, 1H), 2.18 (d, J=7.5 Hz, 1H), 1.99 (d, J=10.0 Hz, 1H), 1.76-1.68 (m, 5H), 1.60 (d, J=6.9 Hz, 3H), 1.30 (s, 3H). LC/MS (ESI) m/z: 596 (M+H)⁺. RT (Method A): 1.71 min.

### Scheme 41. Synthesis of (2S,4R)-N-((R)-1-(1H-pyrrolo[3,2-c]pyridin-2-yl)ethyl)-1-((9,9-difluoro-9H-fluorene-4-carbonyl)glycyl)-4-(difluoromethoxy)pyrrolidine-2-carboxamide (Compound 134)

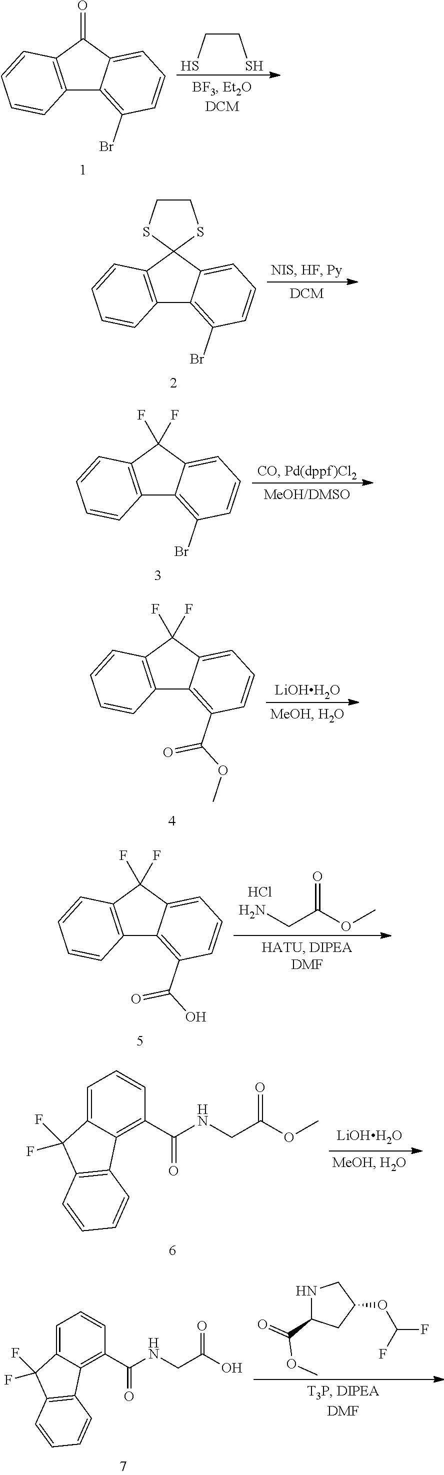

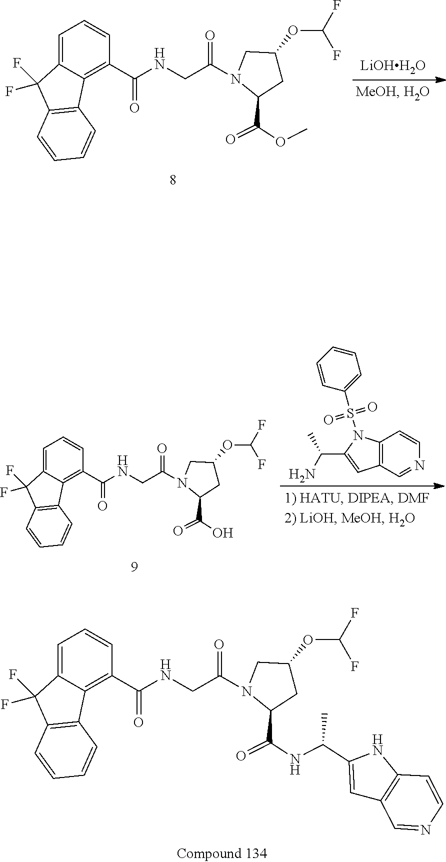

### Step 1: 4-Bromospiro[fluorene-9,2′-[1,3]dithiolane](2)

To a solution of 4-bromo-9H-fluoren-9-one (2.0 g, 7.72 mmol) in dichloromethane (20 mL) was added ethane-1,2-dithiol (3.64 g, 38.6 mmol) and BF₃·Et₂O (4.8 mL) at 0° C. The mixture was stirred at room temperature under N₂ atmosphere for 2 hours. The mixture was diluted with water and extracted with dichloromethane (2×50 mL). The combined organic layers were washed with brine, dried over anhydrous Na₂SO₄, filtered, and concentrated to dryness under reduced pressure. The residue was purified by flash chromatography (silica gel, 0-5% EtOAc in PE) to give 4-bromospiro[fluorene-9,2′-[1,3]dithiolane](2.5 g, yield 96.5%) as a yellow solid.

### Step 2: 4-Bromo-9,9-difluoro-9H-fluorene (3)

To a solution of N-Iodosuccinimide (9.1 g, 40.4 mmol) in dichloromethane (40 mL) was added dropwise HF-pyridine (5.1 mL) at −78° C. and the mixture was stirred for 10 mins. The reaction mixture was added a solution of 4-bromospiro[fluorene-9,2′-[1,3]dithiolane](2.5 g, 7.48 mmol) in dichloromethane (30 mL) drop-wisely and the mixture was stirred at −78° C. for 3 hours. The reaction mixture was quenched with water and extracted with dichloromethane. The combined organic layers were washed with brine, dried over anhydrous Na₂SO₄, filtered, and concentrated to dryness under reduced pressure. The residue was purified by flash chromatography (silica gel, 0-5% EtOAc in PE) to give 4-bromo-9,9-difluoro-9H-fluorene (1.1 g, yield 52.4%) as a yellow solid.

### Step 3: Methyl 9,9-difluoro-9H-fluorene-4-carboxylate (4)

To a solution of 4-bromo-9,9-difluoro-9H-fluorene (500 mg, 1.78 mmol) in MeOH (3 mL)/DMSO (3 mL) was added Pd(dppf)C₁₂(260 mg, 0.35 mmol) and triethylamine (540 mg, 5.34 mmol). The mixture was stirred at 80° C. under CO atmosphere overnight. The reaction was quenched with saturated aq. NH₄Cl solution. The resulting mixture was extracted with EtOAc. The combined organic layers were washed with water and brine, dried over Na₂SO₄, filtered, and concentrated under reduced pressure.

The residue was purified by flash chromatography (silica gel, 0-15% EtOAc in PE) to give methyl 9,9-difluoro-9H-fluorene-4-carboxylate (145 mg, yield 31.3%) as a white solid.

### Step 4: 9,9-Difluoro-9H-fluorene-4-carboxylic acid (5)

To a solution of methyl 9,9-difluoro-9H-fluorene-4-carboxylate (145 mg, 0.56 mmol) in MeOH (2 mL) and water (1 mL) was added LiOH H₂O (97 mug, 2.24 mmol) at 0° C. and the mixture was stirred at room temperature for 16 hours. The mixture was acidified with 1N aq. HCl solution to pH<3 and extracted with EtOAc. The combined organic layers were concentrated to dryness under reduced pressure to give 9,9-difluoro-9H-fluorene-4-carboxylic acid (128 mg, yield 93.4%) as a white solid, which was used directly in next step without further purification. LC/MS (ESI) (m/z): 247(M+H)⁺.

### Step 5: Methyl (9,9-difluoro-9H-fluorene-4-carbonyl)glycinate (6)

To a solution of 9,9-difluoro-9H-fluorene-3-carboxylic acid (120 mg, 0.49 mmol) in EtOAc (2 mL) was added methyl glycinate hydrochloride (73 mg, 0.584 mmol), HATU (277 mg, 0.74 mmol) and DIPEA (314 mg, 2.45 mmol) and the reaction mixture was stirred at room temperature for 1 hour. The mixture was diluted with EtOAc and washed with saturated aq. NaHCO₃ solution. The aqueous layer was extracted with EtOAc, and the combined organic layers were washed with brine, dried over anhydrous Na₂SO₄, filtered, and concentrated under reduced pressure to dryness. The residue was purified by flash chromatography (silica gel, 0-15% EtOAc in PE) to give methyl (9,9-difluoro-9H-fluorene-4-carbonyl)glycinate (60 mg, yield 34.3%) as a white solid. LC/MS (ESI) m/z: 318 (M+H)⁺.

### Step 6: (9,9-Difluoro-9H-fluorene-4-carbonyl)glycine (7)

To a mixture of methyl (9,9-difluoro-9H-fluorene-4-carbonyl)glycinate (60 mg, 0.18 mmol) in MeOH/THF/water (2 mL, 2/1/1) was added LiOH·H₂O (32 mg, 0.72 mmol) and the reaction mixture was stirred at room temperature for 4 hours. The mixture was acidified with IN aq. HCl to pH˜4 and extracted with EtOAc twice. The combined organic layers were washed with brine, dried over anhydrous Na₂SO₄, filtered, and concentrated under reduced pressure to dryness to afford (9,9-difluoro-9H-fluorene-4-carbonyl)glycine (45 mg, yield 82.5%) as a white solid, which was used directly in next step without further purification. LC/MS (ESI) m/z: 304 (M+H)⁺.

### Step 7: Methyl (2S,4R)-1-((9,9-difluoro-9H-fluorene-4-carbonyl)glycyl)-4-(difluoromethoxy)pyrrolidine-2-carboxylate (9)

To a solution of (9,9-difluoro-9H-fluorene-4-carbonyl)glycine (45 mg, 0.15 mmol), methyl (2S,4R)-4-(difluoromethoxy)pyrrolidine-2-carboxylate (43 mg, 0.15 mmol) and DIPEA (96 mg, 0.75 mmol) in DMF (2 mL) was added T₃P (142 mg, 0.23 mmol, 50% wt. in DMF) and the reaction mixture was stirred at room temperature for 1 hour. The mixture was diluted with EtOAc and washed with saturated aq. NaHCO₃ solution. The aqueous layer was extracted with EtOAc, and the combined organic layers were washed with brine, dried over anhydrous Na₂SO₄, filtered, and concentrated under reduced pressure to dryness. The residue was purified by flash chromatography (silica gel, 0-4% MeOH in DCM) to give methyl (2S,4R)-1-((9,9-difluoro-9H-fluorene-4-carbonyl) glycyl)-4-(difluoromethoxy)pyrrolidine-2-carboxylate (56 mg, yield 77.8%) as a colorless oil. LC/MS (ESI) m/z: 481 (M+H)⁺.

### Step 8: (2S,4R)-1-((9,9-difluoro-9H-fluorene-4-carbonyl)glycyl)-4-(difluoromethoxy) pyrrolidine-2-carboxylic acid (10)

To a solution of methyl (2S,4R)-1-((9,9-difluoro-9H-fluorene-4-carbonyl)glycyl)-4-(difluoromethoxy)pyrrolidine-2-carboxylate (56 mg, 0.12 mmol) in MeOH/THF/H₂O (2 mL, 2/2/1) was added LiOH·H₂O (20 mg, 0.48 mmol) and the reaction mixture was stirred at room temperature for 4 hours. The mixture was acidified with 1N aq. HCl to pH˜4 and extracted with ethyl acetate twice. The combined organic layers were washed with brine, dried over anhydrous Na₂SO₄, filtered, and concentrated under reduced pressure to dryness to afford (2S,4R)-1-((9,9-difluoro-9H-fluorene-4-carbonyl)glycyl)-4-(difluoromethoxy)pyrrolidine-2-carboxylic acid (46 mg, yield 84.6%) as a white solid, which was used directly in next step without further purification. LC/MS (ESI) m/z: 467 (M+H)⁺.

### Step 9: (2S,4R)-N-((R)-1-(1H-pyrrolo[3,2-c]pyridin-2-yl)ethyl)-1-((9,9-difluoro-9H-fluorene-4-carbonyl)glycyl)-4-(difluoromethoxy)pyrrolidine-2-carboxamide (Compound 134)

To a mixture of (R)-1-(1-(phenylsulfonyl)-1H-pyrrolo[3,2-c]pyridin-2-yl)ethan-1-amine (15 mg, 0.049 mmol) and (2S,4R)-1-((9,9-difluoro-9H-fluorene-4-carbonyl)glycyl)-4-(difluoromethoxy)pyrrolidine-2-carboxylic acid (23 mg, 0.049 mmol) in DMF (0.6 mL) was added DIPEA (38 mg, 0.29 mmol) and HATU (20 mg, 0.054 mmol) under N₂ atmosphere and the reaction mixture was stirred at room temperature for 1 hours. To the mixture was added LiOH (4.8 mg, 0.20 mmol), MeOH (0.5 mL) and water (0.1 mL) and the mixture was stirred at room temperature for 6 hours. The mixture was diluted with EtOAc and washed with saturated aq·NaHCO₃ solution and brine, dried over anhydrous Na₂SO₄, filtered, and concentrated under reduced pressure to dryness. The residue was purified by prep-TLC (DCM:MeOH=7:1) and further purified by prep-HPLC to give Compound 134 (0.71 mg, yield 2.4%) as a white solid. ¹H NMR (400 MHz, CD₃OD): δ 8.55 (s, 1H), 7.95 (d, J=7.5 Hz, 1H), 7.76 (d, J=4.4 Hz, 2H), 7.67 (d, J=7.4 Hz, 1H), 7.48 (d, J=7.4 Hz, 2H), 7.24 (t, J=7.6 Hz, 1H), 7.17 (t, J=7.5 Hz, 1H), 6.95 (d, J=6.3 Hz, 1H), 6.74-6.37 (m, 2H), 5.34 (d, J=6.2 Hz, 1H), 5.10 (s, 1H), 4.61 (t, J=7.7 Hz, 1H), 4.44 (d, J=16.7 Hz, 1H), 4.28 (d, J=16.7 Hz, 1H), 4.04 (d, J=7.6 Hz, 1H), 3.96 (d, J=11.2 Hz, 1H), 2.49 (d, J=6.8 Hz, 1H), 2.32 (d, J=12.8 Hz, 1H), 1.62 (d, J=6.8 Hz, 3H). LC/MS (ESI) m/z: 610 (M+H)⁺. RT (Method A): 1.84 min.

### Scheme 42. Synthesis of (2S,4R)-N-((1H-pyrrolo[3,2-c]pyridin-2-yl)methyl)-4-(difluoromethoxy)-1-(2-(3-(2,4-difluorophenyl)-5-oxo-5,7-dihydro-6H₁-pyrrolo[3,4-b]pyridin-6-yl)acetyl)pyrrolidine-2-carboxamide (Compound 135)

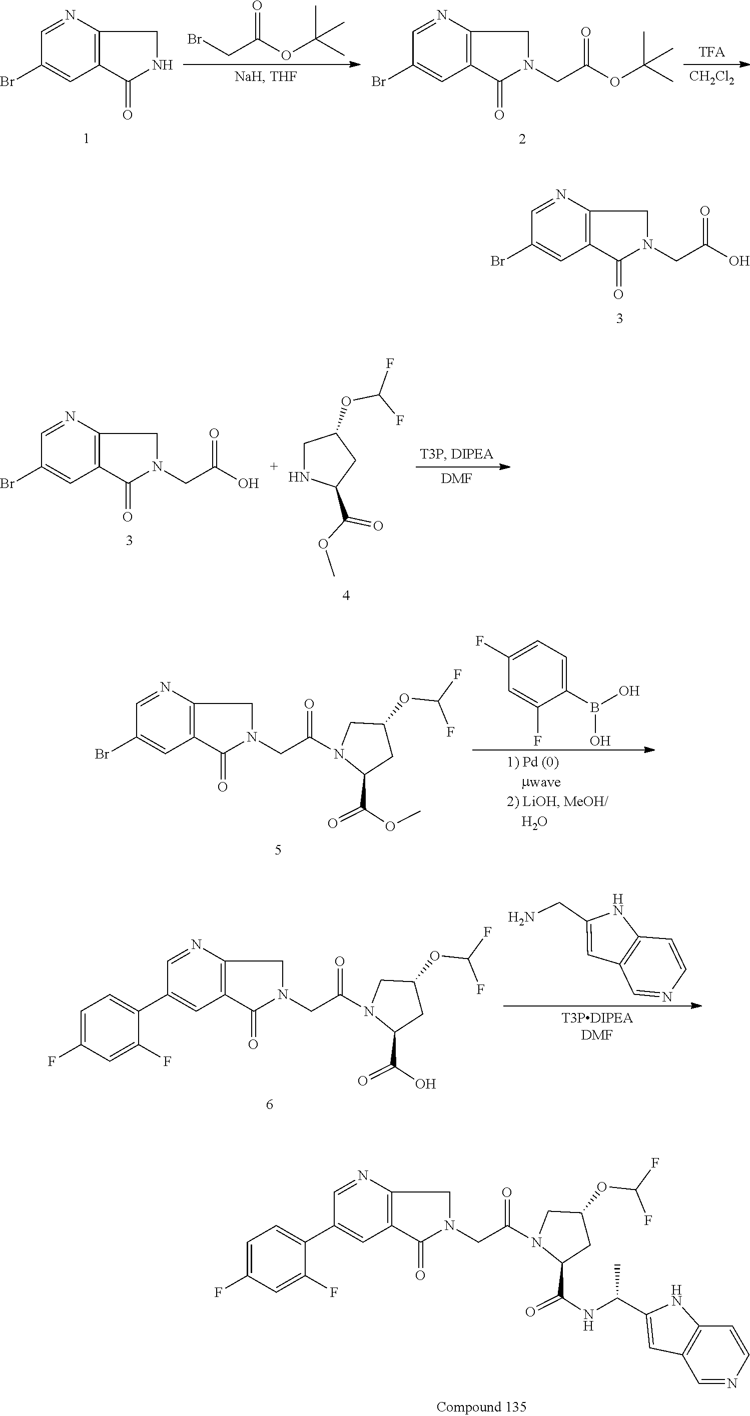

### Step 1: tert-butyl 2-(3-bromo-5-oxo-5,7-dihydro-6H-pyrrolo[3,4-b]pyridin-6-yl)acetate (2)

To a solution of hexane washed sodium hydride (60 mass %) in oil (93.88 mg, 2.347 mmol, 2.0 equiv.) in THF (5 mL) was added 3-bromo-6,7-dihydropyrrolo[3,4-b]pyridin-5-one (250.0 mg, 1.174 mmol, 1.0 equiv.). The reaction mixture was stirred at room temperature for 30 minutes and then cooled to ice-bath temperature. Tert-butyl 2-bromoacetate (0.4578 g, 2.347 mmol, 2.0 equiv.) was added and the reaction mixture was allowed to warm to room temperature and then stirred for 2 hours and then diluted with water (10 mL) and EtOAc (10 mL). The two layers were separated, and the aqueous layer was extracted with EtOAc (10 mL×2). The combined organic extracts were washed with brine, dried over Na₂SO₄, and concentrated. Purification by Combi Flash; 12 g column, solvent A=CH₂Cl₂, solvent B=MeOH, 100% A to 5% B gave tert-butyl 2-(3-bromo-5-oxo-7H-pyrrolo[3,4-b]pyridin-6-yl)acetate (0.3456 g, 1.056 mmol, 90.00% yield). LC/MS (ESI) m/z: 328 (M+H)⁺.

### Step 2: 2-(3-bromo-5-oxo-5,7-dihydro-6H-pyrrolo[3,4-b]pyridin-6-yl)acetic acid (3)

TFA (1.7 mL) was added to a solution of tert-butyl 2-(3-bromo-5-oxo-7H-pyrrolo[3,4-b]pyridin-6-yl)acetate (200.0 mg, 0.6112 mmol, 1.0 equiv.) in CH₂Cl₂ (3 mL) at ice-bath temperature. The reaction mixture was stirred at room temperature for 4 hours and then concentrated to dryness to give 2-(3-bromo-5-oxo-7H-pyrrolo[3,4-b]pyridin-6-yl)acetic acid (0.1567 g, 0.5781 mmol, 94.57% yield) as a white solid, which was used in the next step without further purification. LC/MS (ESI) m/z: 272 (M+H)⁺.

### Step 3: methyl (2S,4R)-1-(2-(3-bromo-5-oxo-5,7-dihydro-6H-pyrrolo[3,4-b]pyridin-6-yl)acetyl)-4-(difluoromethoxy)pyrrolidine-2-carboxylate (5)

To a solution of 2-(3-bromo-5-oxo-7H-pyrrolo[3,4-b]pyridin-6-yl)acetic acid (120.0 mg, 0.4427 mmol, 1.0 equiv.) in DMF (5 mL) was added methyl (2S,4R)-4-(difluoromethoxy)pyrrolidine-2-carboxylate hydrochloride (123.0 mg, 0.5310 mmol, 1.2 equiv.), T3P (398.0 μL, 0.665 mmol, 1.5 equiv., 50 mass % in ethyl acetate), and DIPEA (232.0 μL, 1.33 mmol, 3.0 equiv.). The reaction mixture was stirred at room temperature for 30 minutes. Water (5 mL) was added to the reaction mixture, and the solid was collected by filtration, rinsed with water, and dried to give methyl (2S,4R)-1-[2-(3-bromo-5-oxo-7II-pyrrolo[3,4-b]pyridin-6-yl)acetyl]-4-(difluoromethoxy)pyrrolidine-2-carboxylate (167.0 mg, 0.3726 mmol, 84.17% yield) as a brown solid. LC/MS (ESI) m/z: 449 (M+H)⁺.

### Step 4: (2S,4R)-4-(difluoromethoxy)-1-(2-(3-(2,4-difluorophenyl)-5-oxo-5,7-dihydro-6H-pyrrolo[3,4-b]pyridin-6-yl)acetyl)pyrrolidine-2-carboxylic acid (6)

A mixture of 2,4-difluorophenyl)boronic acid (14.80 mg, 0.09372 mmol, 1.2 equiv.), methyl (2S,4R)-1-[2-(3-bromo-5-oxo-7H-pyrrolo[3,4-b]pyridin-6-yl)acetyl]-4-(difluoromethoxy)pyrrolidine-2-carboxylate (35.00 mg, 0.07809 mmol, 1.0 equiv.), Pd(PPh₃)₂dppf·CH₂Cl₂ (6.440 mg, 0.00781 mmol, 0.1 equiv.), Na₂CO₃ (16.55 mg, 0.1561 mmol, 2 equiv.) in 1,4-dioxane (3 mL), and water (0.3 mL) was stirred in a microwave reactor at 110° C. for 30 min. The reaction filtered through Celite pad. The cake was washed with water and EtOAc. The two layers were separated, and the aqueous layer was extracted with EtOAc, washed with brine, dried over Na₂SO₄, and concentrated to give a brown oil. To a solution of the brown oil in MeOH (1.5 mL) and water (0.5 mL) was added LiOH (2.244 mg, 0.09370 mmol, 2 equiv.). The reaction mixture was stirred at room temperature overnight and then concentrated to remove McOH. The residue was diluted with EtOAc and H₂O. The two layers were separated, and the pH of the aqueous layer was adjusted to 1 using 4M HCl and then extracted with EtOAc. The combined organic extracts were washed with brine, dried over Na₂SO₄, and then concentrated to give (2S,4R)-4-(difluoromethoxy)-1-[2-[3-(2,4-difluorophenyl)-5-oxo-7H-pyrrolo[3,4-b]pyridin-6-yl]acetyl]pyrrolidine-2-carboxylic acid (20.36 mg, 0.04356 mmol, 55.78% yield) as a brown oil, which was used in the next step without further purification. LC/MS (ESI) m/z: 468 (M+H)⁺.

### Step 5: (2S,4R)-N-((1H-pyrrolo[3,2-c]pyridin-2-yl)methyl)-4-(difluoromethoxy)-1-(2-(3-(2,4-difluorophenyl)-5-oxo-5,7-dihydro-6H-pyrrolo[3,4-b]pyridin-6-yl)acetyl)pyrrolidine-2-carboxamide (Compound 135)

To a solution of (2S,4R)-4-(difluoromethoxy)-1-[2-[3-(2,4-difluorophenyl)-5-oxo-7H-pyrrolo[3,4-b]pyridin-6-yl]acetyl]pyrrolidine-2-carboxylic acid (21.24 mg, 0.04544 mmol 1.0 equiv.) in DMF (2 mL) was added 1H-pyrrolo[3,2-c]pyridin-2-ylmethanamine;dihydrochloride (10.00 mg, 0.04543 mmol, 1.0 equiv.), T3P (50 mass %) in ethyl acetate (40.80 NL, 0.0681 mmol, 1.5 equiv.), and DIPEA (23.8 μL, 0.136 mmol, 3.0 equiv.). The reaction mixture was stirred at room temperature for 30 minutes and then purified directly with prep HPLC to give Compound 135 (4.218 mg, 0.007071 mmol, 15.56% yield). LC/MS (ESI) m/z: 597 (M+H)⁺. ¹H NMR (400 MHz, DMSO-d6): δ 11.12 (s, 1H), 8.87 (d, J=11.9 Hz, 1H), 8.84-8.42 (m, 2H), 8.18 (d, J=12.5 Hz, 1H), 8.03 (s, 1H), 7.93 (d, J=5.6 Hz, 1H), 7.78-7.65 (m, 1H), 7.42 (d, J=10.5 Hz, 1H), 7.23 (d, J=10.0 Hz, 2H), 7.01 (d, J=5.7 Hz, 1H), 6.74 (d, J=15.0 Hz, 1H), 6.55 (d, J=16.0 Hz, 3H), 6.30 (s, 1H), 4.92 (s, 1H), 4.68-4.16 (m, 3H), 3.93-3.64 (m, 2H), 2.27 (d, J=10.5 Hz, 1H), 2.11 (d, J=8.1 Hz, 1H). RT (Method A): 1.28 min.

The following compounds were prepared based on Scheme 42:

| Cmpd # | Reactant A | Reactant B | Reactant C | Reactant D | Characterization Data |
|---|---|---|---|---|---|
| 90^{a,b} | 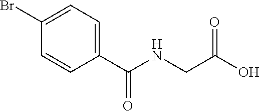 [SMILES: O=C(O)CNC(=O)c1ccc(Br)cc1] | 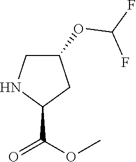 | 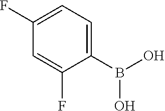 [SMILES: OB(O)c1ccc(F)cc1F] | 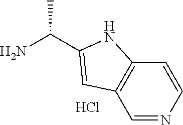 | ¹H NMR (400 MHz, DMSO-d6): δ 11.22 (s, 1H), 8.86 (d, J = 8.0 Hz, IH), 8.77-8.54 (m, 1H), 8.41 (d, J = 8.2 Hz, 1H), 7.91 (dd, J = 8.3, 4.3 Hz, 1H), 7.70-7.47 (m, 1H), 7.32 (ddd, J = 11.5, 9.3, 2.5 Hz, 1H), 6.92 (d, J = 11.8 Hz, 1H), 6.73 (d, J = 11.8 Hz, 1H), 6.54 (d, J = 11.7 Hz, 1H), 6.31 (s, 1H), 5.13 (q, J = 7.3 Hz, 1H), 4.89 (d, J = 4.6 Hz, 1H), 4.83 (s, 1H), 4.61 (t, J = 7.3 Hz, 1H), 4.39 (t, J = 7.6 Hz, 2H), 4.20-4.09 (m, 2H), 3.99 (dd, J = 16.7, 5.6 Hz, 2H), 3.81 (td, J = 10.7, 5.3 Hz, 1H), 3.77-3.65 (m, 1H), 3.54 (dd, J = 12.7, 4.8 Hz, 1H), 2.55- 2.34 (m, 2H), 2.32-2.16 (m, 1H), 2.09 (dt, J = 13.0, 5.7 Hz, |
| | | | | | 1H), 1.42 (d, J = 6.9 Hz, 3H). LC/MS (ESI) m/z: 598 (M + H)⁺. |
| | | | | | RT (Method A): 1.40 min. |
| 120^{a} | 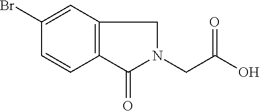 [SMILES: O=C(O)CN1Cc2cc(Br)ccc2C1=O] | 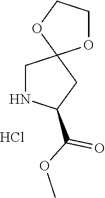 | 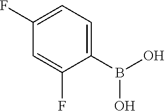 [SMILES: OB(O)c1ccc(F)cc1F] | 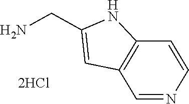 [SMILES: NCc1cc2cnccc2[nH]1 ; "2HCl"] | ¹H NMR (400 MHz, DMSO-d6): δ 11.11 (s, IH), 9.17-8.37 (m, 1H), 8.10 (s, 1H), 7.93 (s, 1H), 7.75 (d, J = 7.9 Hz, 1H), 7.67 (d, J = 11.1 Hz, 1H), 7.60 (d, J = 8.4 Hz, 2H), 7.36 (t, J = 10.3 Hz, 1H), 7.19 (t, J = 8.7 Hz, 1H), 7.02 (s, 1H), 6.33 (s, 1H), 4.85-4.07 (m, 5H), 4.03-3.64 (m, 3H), 3.60 (d, J = 10.2 Hz, 1H), 2.38-2.14 (m, 1H), 2.05 (dd, J = 13.3, 7.3 Hz, 1H), 1.58-1.16 (m, 1H). LC/MS (ESI) m/z: 588 (M + H)⁺. RT (Method A): 1.34 min. |

| | | | | |
|---|---|---|---|---|
| 138^{c} | 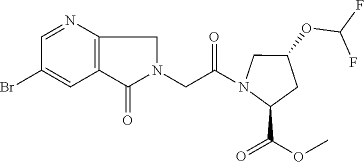 | 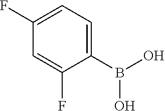 [SMILES: OB(O)c1ccc(F)cc1F] | 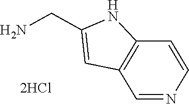 [SMILES: NCc1cc2cnccc2[nH]1 ; "2HCl"] | ¹H NMR (400 MHz, DMSO-d6): δ 11.16 (s, 1H), 9.04 (d, J = 2.2 Hz, 1H), 8.80-8.46 (m, 2H), 8.44-8.19 (m, 2H), 8.25- 7.65 (m, 2H), 7.46-7.17 (m, 1H), 7.21-6.91 (m, 1H), 6.74 (d, J = 15.4 Hz, 1H), 6.47-5.95 (m, 1H), 5.01-4.87 (m, 1H), 4.68-4.43 (m, 2H), 4.39 (dd, J = 14.5, 5.7 Hz, 2H), 3.85 (dd, J = 11.5, 4.6 Hz, 1H), 3.76 (d, J = 10.8 Hz, 1H), 2.37-2.24 (m, 1H), 2.16-2.06 (m, 1H), 1.38 (s, 1H), 1.16 (d, J = 4.9 Hz, 1H). LC/MS (ESI) m/z: 579 (M + H)⁺. RT (Method A): 1.27 min. |

^{a}Steps 3-5 only.
^{b}HATU was used in place of T₃P in Step 3.
^{c}Steps 4 and 5 only.

### Scheme 43. Synthesis of (2S,4R)-4-(difluoromethoxy)-1-((phenoxathiine-3-carbonyl)glycyl)-N-(thieno[3,2-c]pyridin-2-ylmethyl)pyrrolidine-2-carboxamide (Compound 142)

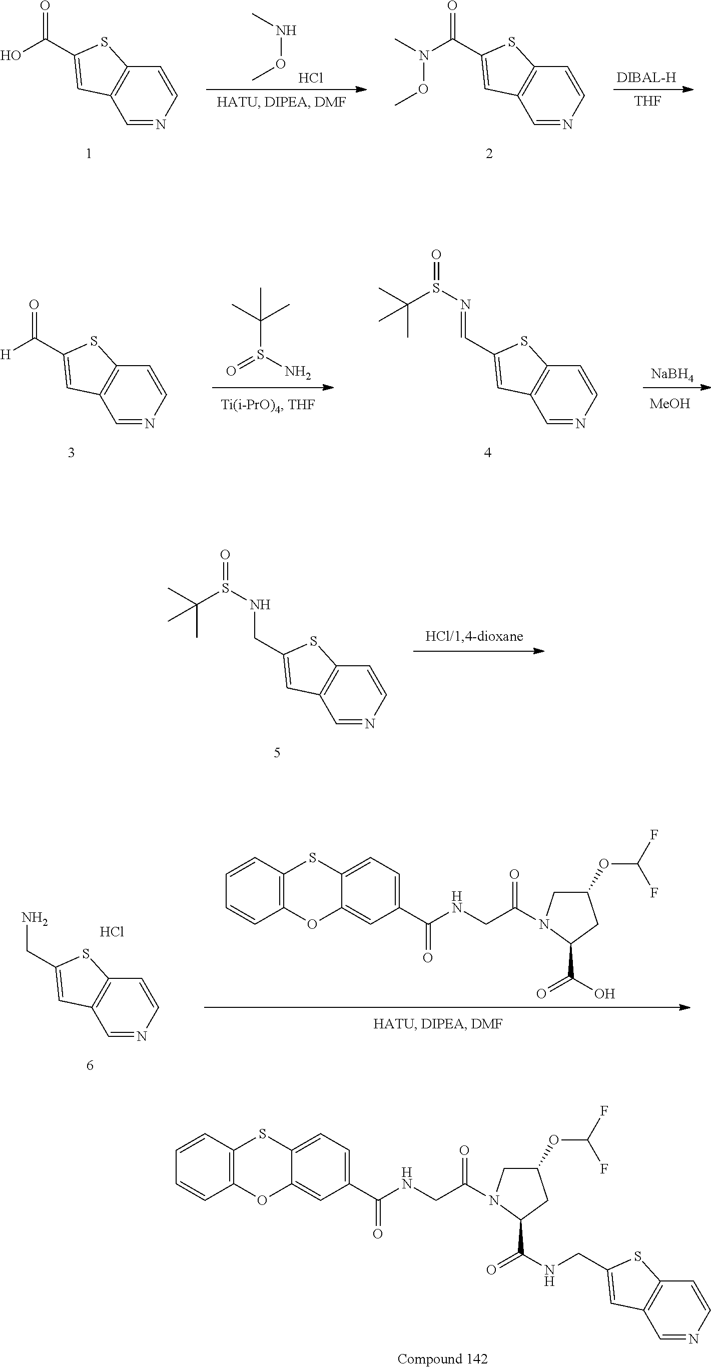

### Step 1: N-methoxy-N-methylthieno[3,2-c]pyridine-2-carboxamide (2)

To a solution of thieno[3,2-c]pyridine-2-carboxylic acid (900 mg, 5.03 mmol) in DMF (10 mL) was added N,O-dimethylhydroxylamine hydrochloride (735 mg, 7.54 mmol), HATU (2.86 g, 7.54 mmol) and DIPEA (0.89 mL, 15.1 mmol) and the reaction mixture was stirred at room temperature for 1 hour. The mixture was diluted with EtOAc and washed with saturated aq·NaHCO₃ solution. The layers were separated, and the aqueous layer was extracted with EtOAc. The combined organic layers were washed with brine, dried over anhydrous Na₂SO₄, filtered, and concentrated under reduced pressure to dryness. The residue was purified by flash chromatography (silica gel. 0-30% EtOAc in PE) to give N-methoxy-N-methylthieno[3,2-c]pyridine-2-carboxamide (1.1 g, yield 98.6%) as a white solid. LC/MS (ESI) m/z: 223 (M+H)⁺.

### Step 2: Thieno[3,2-c]pyridine-2-carbaldehyde (3)

To a solution of N-methoxy-N-methylthieno[3,2-c]pyridine-2-carboxamide (300 mg, 1.68 mmol) in THF (3 mL) was added DIBAL-H (5 mL, 5.06 mmol, 1 mol/L) drop-wisely at 0° C. and the mixture was stirred at this temperature for 1 hour. The mixture was quenched with saturated potassium sodium tartrate solution and stirred for 2 hours. The mixture was extracted with EtOAc twice.

The combined organic layers were washed with brine, dried over anhydrous Na₂SO₄, filtered, and concentrated under reduced pressure to dryness. The residue was purified by flash chromatography (silica gel, 0-5% McOH in DCM) to give thieno[3,2-c]pyridine-2-carbaldehyde (120 mg, yield 43.8%) as a colorless oil. LC/MS (ESI) m/z: 164 (M+H)⁺.

### Step 3: (E)-2-methyl-N-(thieno[3,2-c]pyridin-2-ylmethylene)propane-2-sulfinamide (4)

To a mixture of thieno[3,2-c]pyridine-2-carbaldehyde (120 mg, 0.74 mmol) and (R)-2-methylpropane-2-sulfinamide (223 mg, 1.84 mmol) in THF (3 mL) was added titanium tetraisopropanolate (0.2 mL) at 0° C. and the mixture was stirred at 70° C. for 6 hours. The mixture was quenched with ice-water and the slurry was filtered. The filtrate was extracted with EtOAc twice. The combined organic layers were washed with brine, dried over anhydrous Na₂SO₄, filtered, and concentrated under reduced pressure to dryness. The residue was purified by flash chromatography (silica gel, 0-5% MeOH in DCM) to give (E)-2-methyl-N-(thieno[3,2-c]pyridin-2-ylmethylene)propane-2-sulfinamide (80 mg, yield 40.6%) as a colorless oil. LC/MS (ESI) m/z: 267 (M+H)⁺.

### Step 4: 2-Methyl-N-(thieno[3,2-c]pyridin-2-ylmethyl)propane-2-sulfinamide (4)

To a solution of (E)-2-methyl-N-(thieno[3,2-c]pyridin-2-ylmethylene)propane-2-sulfinamide (80 mg, 0.30 mmol) in MeOH (3 mL) was added NaBH₄ (45 mg, 1.2 mmol) and the reaction mixture was stirred at 0° C. for 1 hour. The mixture was diluted with water and extracted with EtOAc twice. The combined organic layers were washed with brine, dried over anhydrous Na₂SO₄ filtered, and concentrated under reduced pressure to dryness. The residue was purified by flash chromatography (silica gel, 0-5% MeOH in DCM) to give 2-methyl-N-(thieno[3,2-c]pyridin-2-ylmethyl)propane-2-sulfinamide (50 mg, yield 63.2%) as a colorless oil. LC/MS (ESI) m/z: 269 (M+H)⁺.

### Step 5: Thieno[3,2-c]pyridin-2-ylmethanamine hydrochloride (5)

A solution of 2-methyl-N-(thieno[3,2-c]pyridin-2-ylmethyl)propane-2-sulfinamide (50 mg, 0.18 mmol) in HCl/1,4-dioxane (1 mL, 4M) was stirred under N₂ atmosphere at room temperature for 2 hours. The reaction mixture was concentrated under reduced pressure to dryness to give thieno[3,2-c]pyridin-2-ylmethanamine hydrochloride (35 mg, yield 97.3%) as a white solid, which was used directly in the next step without further purification. LC/MS (ESI) in/z: 165 (M+H).

### Step 6: (2S,4R)-4-(difluoromethoxy)-1-((phenoxathiine-3-carbonyl)glycyl)-N-(thieno [3,2-c]pyridin-2-ylmethyl)pyrrolidine-2-carboxamide (Compound 142)

To a mixture of (2S,4R)-4-(difluoromethoxy)-1-((phenoxathiine-3-carbonyl)glycyl) pyrrolidine-2-carboxylic acid (20 mg, 0.045 mmol) and thieno[3,2-c]pyridin-2-ylmethanamine hydrochloride (9.2 mg, 0.045 mmol) in DMF (1 mL) was added DIPEA (17.4 mg, 0.14 mmol) and HATU (25.6 mg, 0.07 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-HPLC to give Compound 142 (2.1 mg, yield 7.6%) as a white solid. ¹H NMR (400 MHz, CD₃OD): δ 8.86 (s, 1H), 8.22 (d, J=5.7 Hz, 1H), 7.79 (d, J=5.3 Hz, 1H), 7.49 (d, J=8.4 Hz, 1H), 7.41 (s, 2H), 7.22-7.13 (m, 3H), 7.05 (dd, J=16.3, 8.0 Hz, 2H), 6.51 (t, J=74.5 Hz, 1H), 5.02 (s, 1H), 4.77 (s, 1H), 4.62 (dd, J=19.6, 11.8 Hz, 2H), 4.19 (s, 2H), 4.00 (d, J=7.3 Hz, 1H), 3.88 (d, J=11.2 Hz, 1H), 2.49 (dd, J=13.8, 7.7 Hz, 1H), 2.32-2.21 (m, 1H). LC/MS (ESI) m/z: 611 (M+H)⁺. RT (Method A): 1.57 min.

### Scheme 44. Synthesis of (2S,3R)-N-((1H-pyrrolo[3,2-c]pyridin-2-yl)methyl)-3-hydroxy-1-((4-phenoxybenzoyl)glycyl)pyrrolidine-2-carboxamide (Compound 151)

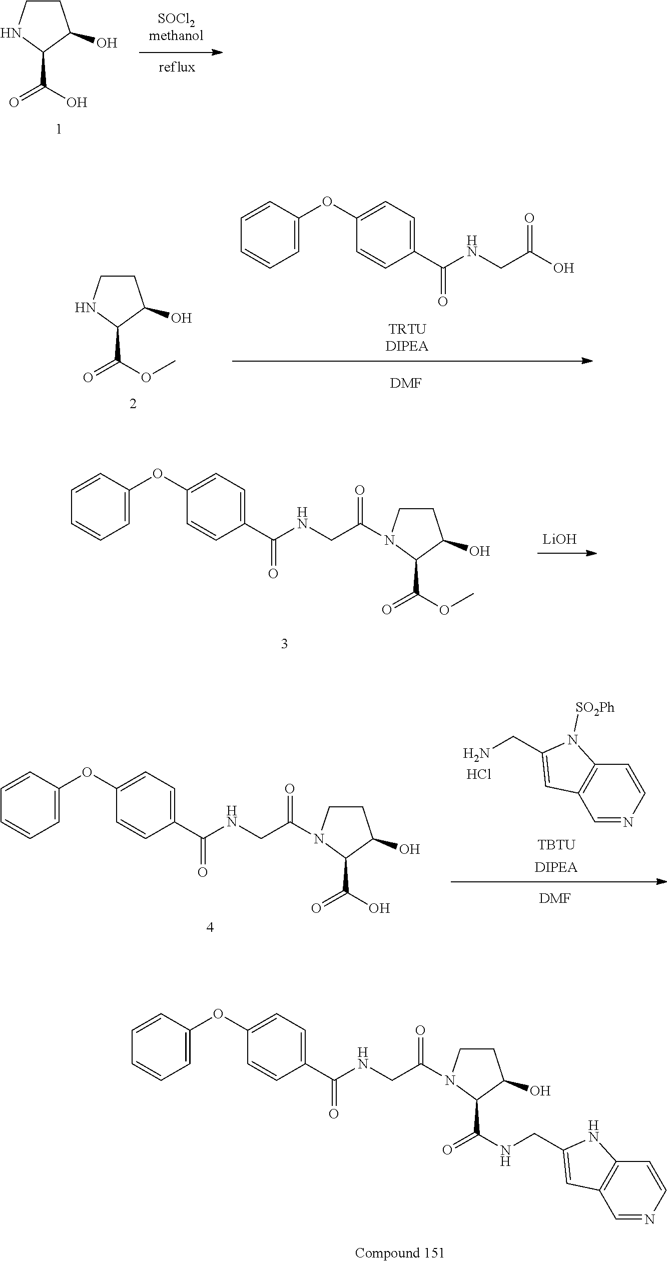

### Step 1: Methyl (2S,3R)-3-hydroxypyrrolidine-2-carboxylate (2)

To a solution of (2S,3R)-3-hydroxypyrrolidine-2-carboxylic acid (100 mg, 0.76260 mmol) in methanol (10 mL) was added thionyl chloride (0.064 mL, 1.15 equiv.). The reaction mixture was heated to 70° C. for overnight to give a wine color solution. The solvent was removed by concentration under reduced pressure, the resulting residue was azeotroped with methanol twice to give methyl (2S,3R)-3-hydroxypyrrolidine-2-carboxylate (160 mg, 86.7% yield) as a pink wax. LC/MS (ESI) m/z: 146 (M+H)⁺.

### Step 2: Methyl (2S,3R)-3-hydroxy-1-((4-phenoxybenzoyl)glycyl)pyrrolidine-2-carboxylate (3)

To a solution of 2-[(4-phenoxybenzoyl)amino]acetic acid (0.085 g, 0.31 mmol), methyl (2S,3R)-3-hydroxypyrrolidine-2-carboxylate (30 mg, 0.20667 mmol) and HATU (100 mg, 0.31 mmol) was added DIPEA (0.11 mL, 0.63 mmol) at room temperature. The reaction mixture was stirred for 18 hours. The reaction mixture was diluted with water and the crude material was concentrated and purified on a C-18 column (10 to 100% acetonitrile in water) to give methyl (2S,3R)-3-hydroxy-1-[2-[(4-phenoxybenzoyl)amino]acetyl]pyrrolidine-2-carboxylate (160 mg, 0.2 mmol, 97.2% yield) as a yellow wax. LC/MS (ESI) m/z: 399 (M+H)⁺.

### Step 3: (2S,3R)-3-Hydroxy-1-((4-phenoxybenzoyl)glycyl)pyrrolidine-2-carboxylic acid (4)

To a solution of methyl (2S,3R)-3-hydroxy-1-[2-[(4-phenoxybenzoyl)amino]acetyl]pyrrolidine-2-carboxylate (84 mg, 0.2108 mmol) in MeOH (1 mL) and water (1 mL) was added LiOH·H₂O (88 mg. 2.1 mmol). The reaction mixture was stirred at room temperature for 18 hours. The reaction mixture was lyophilized for 72 hours to give (2S,3R)-3-hydroxy-1-[2-[(4-phenoxybenzoyl)amino]acetyl]pyrrolidine-2-carboxylic acid (82 mg, >theory) as a white solid. This material was used in next step without further purification. LC/MS (ESI) m/z=385 (M+H)⁺.

### Step 4: (2S,3R)-3-Hydroxy-1-[2-[(4-phenoxybenzoyl)amino]acetyl]-N-(1H-pyrrolo[3,2-c]pyridin-2-ylmethyl)pyrrolidine-2-carboxamide (Compound 151)

To a solution of (2S,3R)-3-hydroxy-1-[2-[(4-phenoxybenzoyl)amino]acetyl]pyrrolidine-2-carboxylic acid (82 mg, 0.2133 mmol), TBTU (200 mg, 0.32 mmol) and [1-(benzenesulfonyl)pyrrolo[3,2-c]pyridin-2-yl]methanamine hydrochloride (83 mg, 0.26 mmol) in DMF (1 mL) at room temperature. The reaction mixture was stirred for 30 minutes and diluted with water. The mixture was extracted with ethyl acetate twice. The two-layer mixture was stirred at room temperature for 18 hours. LC/MS analysis indicated majority of product lost protection mainly due to extra amount of base used in previous step. The mixture was first purified on a C-18 column (0 to 100% acetonitrile in water) and the enriched fractions were concentrated together. The remaining residue was purified again on prep-HPLC to give Compound 151 (2 mg, 1.8% yield) as a white solid. ¹H-NMR (400 MHz, DMSO-d6): δ 8.61 (m, 1H), 8.50 (t, J=9.8 Hz, 1H), 8.26 (d, J=7.8 Hz, 1H), 8.00 (dd, J=18.5, 5.6 Hz, 1H), 7.88-7.76 (m, 2H), 7.38 (t, J=7.7 Hz, 2H), 7.26-7.11 (m, 2H), 6.99 (m, 3H), 6.40 (m, 1H), 4.59-4.21 (m, 2H), 4.15-3.89 (m, 2H), 3.66 (s, 2H), 2.14-1.68 (m, 2H). LC/MS (ESI) m/z=514 (M+H)⁺. RT (Method A): 1.23 min.

The following compounds were prepared based on Scheme 44:

| Cmpd # | Reactant A | Reactant B | Reactant C | Characterization Data |
|---|---|---|---|---|
| 214 | 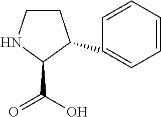 [SMILES: O=C(O)[C@H]1NCC[C@@H]1c1ccccc1] | 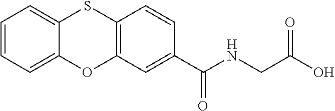 [SMILES: O=C(O)CNC(=O)c1ccc2c(c1)Oc1ccccc1S2] | 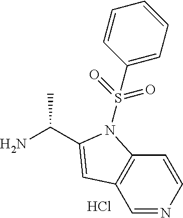 | ¹H-NMR (400 MHz, DMSO-d6) δ 11.06 (s, 1H), 8.72 (t, J = 5.8 Hz, 2H), 8.58 (s, 1H), 8.52 (t, J = 5.9 Hz, 1H), 7.91 (d, J = 5.6 Hz, 1H), 7.62-7.48 (m, 3H), 7.33 (d, J = 8.0 Hz, 1H), 7.30-7.15 (m, 4H), 7.07 (dd, J = 7.8, 4.3 Hz, 2H), 6.15 (s, 1H), 4.61-4.26 (m, 4H), 4.22 (dd, J = 16.9, 5.8 Hz, 1H), 4.02 (dd, J = 16.6, 5.6 Hz, 1H), 3.82-3.57 (m, 2H), 3.41 (q, J = 7.0 Hz, 1H), 2.37-2.26 (m, 1H), 2.02 (dd, J - 12.4, 7.3 Hz, 1H). LC/MS (ESI) m/z: 604 (M + H)⁺. RT (Method A): 1.61 min. |
| 242 | 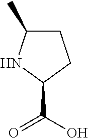 | 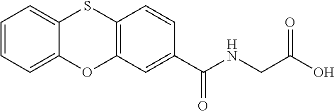 [SMILES: O=C(O)CNC(=O)c1ccc2c(c1)Oc1ccccc1S2] | 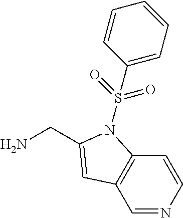 [SMILES: NCc1cc2cnccc2n1S(=O)(=O)c1ccccc1] | ¹H-NMR (400 MHz, CD₃OD): δ 8.51 (m, 3H), 7.89 (s, 1H), 7.53-7.23 (m, 2H), 7.20-7.03 (m, 2H), 7.03-6.89 (m, 2H), 6.56 (s, 1H), 4.57 (d, J = 4.7 Hz, 2H), 4.50 (s, 2H), 4.11 (s, 2H), 3.88 (d, J = 6.9 Hz, 1H), 3.81-3.65 (m, 2H), 2.37- 2.23 (m, 1H), 2.22-2.10 (m, 1H), 1.66 (dd, J = 12.4, 8.3 Hz, 1H), 1.13 (d, J = 6.7 Hz, 3H). LC/MS (ESI) m/z: 542.3 (M + H)⁺. RT (Method A): 1.30 min. |
| 243 | 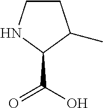 | 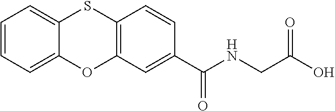 [SMILES: O=C(O)CNC(=O)c1ccc2c(c1)Oc1ccccc1S2] | 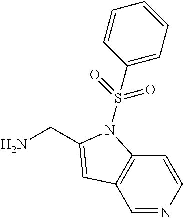 [SMILES: NCc1cc2cnccc2n1S(=O)(=O)c1ccccc1] | ¹H-NMR (400 MHz, CD₃OD): δ 8.64 (m, 4H), 7.67-7.36 (m, 2H), 7.32-7.00 (m, 3H), 4.59-4.37 (m, 4H), 4.27 (d, J = 5.7 Hz, 2H), 2.12 (m, 4H), 1.47 (d, J = 6.5 Hz, 3H). LC/MS (ESI) m/z: 542.3 (M + H)⁺. RT (Method A): 1.29 min. |
| 261 | 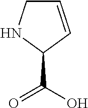 | 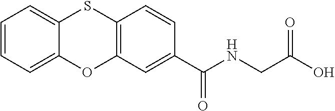 [SMILES: O=C(O)CNC(=O)c1ccc2c(c1)Oc1ccccc1S2] | 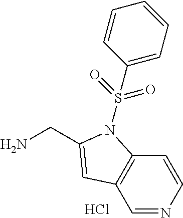 [SMILES: NCc1cc2cnccc2n1S(=O)(=O)c1ccccc1 ; Cl] | ¹H-NMR (400 MHz, CD₃OD): δ 8.49 (m, 1H), 7.90 (d, J = 6.0 Hz, 1H), 7.38 (d, J = 8.3 Hz, 1H), 7.30 (s, 3H), 7.21 (d, J = 6.0 Hz, 1H), 7.16-6.87 (m, 3H), 6.50 (m, 1H), 6.05 (d, J = 6.4 Hz, 1H), 5.84 (s, 1H), 5.11 (s, 2H), 4.21-4.01 (m, 2H), 1.4 (m, 2H). LC/MS (ESI) m/z = 526 (M + H)⁺. RT (Method A): 1.28 min. |

### Scheme 45. Synthesis of (2S,4R)-N-((1H-pyrrolo[3,2-c]pyridin-2-yl)methyl)-4-(difluoromethoxy)-1-((3-(4-fluorophenoxy)benzoyl)glycyl)pyrrolidine-2-carboxamide (Compound 154)

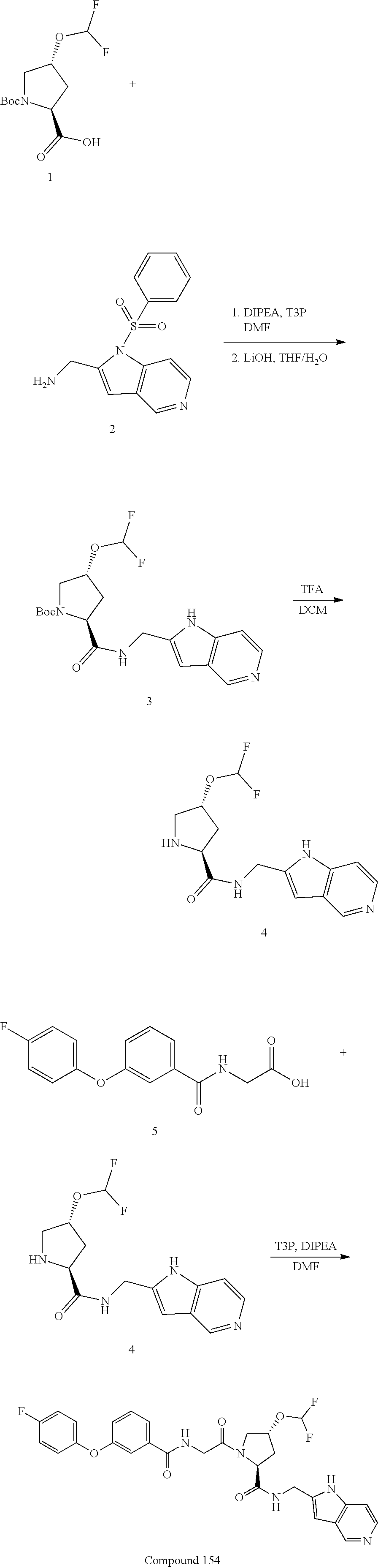

### Steps 1 and 2: tert-butyl (2S,4R)-2-(((1H-pyrrolo[3,2-c]pyridin-2-ylmethyl)carbamoyl)-4-(difluoromethoxy)pyrrolidine-1-carboxylase (3)

To a solution of (2S,4R)-1-tert-butoxycarbonyl-4-(difluoromethoxy)pyrrolidine-2-carboxylic acid (0.1129 g, 0.4014 mmol, 1.0 equiv.) in DMF (2 mL) was added [1-(benzenesulfonyl)pyrrolo[3,2-c]pyridin-2-yl]methanamine hydrochloride (0.1300 g, 0.4015 mmol, 1.0 equiv.), T3P (50 mass %) in ethyl acetate (361 μL, 0.603 mmol, 1.5 equiv.), and DIPEA (0.210 mL, 1.20 mmol, 3.0 equiv.). The reaction mixture was stirred at room temperature for 30 minutes and then diluted with water. The filtrate was decanted, and the residue was washed with water, dried over Na₂SO₄, and concentrated to give a brown oil, which was used in the next reaction without further purification. To the brown oil obtained above in THE (1.5 mL) and water (0.5 mL) was added LiOH (19.23 mg, 0.8030 mmol, 2 equiv.), the reaction mixture was stirred at room temperature overnight and then concentrated to remove THF. The pH was adjusted to 1 using 4M HCl, concentrated, and purified by prep HPLC to give tert-butyl (2S,4R)-4-(difluoromethoxy)-2-(1H-pyrrolo[3,2-c]pyridin-2-ylmethylcarbamoyl)pyrrolidine-1-carboxylate (0.1150 g, 0.2802 mmol, 69.79% yield. LC/MS (ESI) m/z: 411 (M+H)⁺.

### Step 3: (2S,4R)-N-((1H-pyrrolo[3,2-c]pyridin-2-yl)methyl)-4-(difluoromethoxy)pyrrolidine-2-carboxamide (4)

TFA (0.5 mL) was added to a solution of tert-butyl (2S,4R)-4-(difluoromethoxy)-2-(1H-pyrrolo[3,2-c]pyridin-2-ylmethylcarbamoyl)pyrrolidine-1-carboxylate (0.1000 g, 0.2437 mmol, 1.0 equiv.) in CH₂Cl₂ (1 mL) at ice-bath temperature. The reaction mixture was stirred at room temperature for 3 hours and then concentrated to dryness to give (2S,4R)-4-(difluoromethoxy)-N-(1H-pyrrolo[3,2-c]pyridin-2-ylmethyl)pyrrolidine-2-carboxamide (75 mg, 0.2417 mmol, 100 mass %, 99.19% yield) as a clear oil, which was used in the next step without further purification. LC/MS (ESI) m/z: 311(M+H)⁺.

### Step 4: (2S,4R)-N-((JH-pyrrolo[3,2-c]pyridin-2-yl)methyl)-4-(difluoromethoxy)-1-((3-(4-fluorophenoxy)benzoyl)glycyl)pyrrolidine-2-carboxamide (Compound 154)

To a solution of 2-[[3-(4-fluorophenoxy)benzoyl]amino]acetic acid (10.00 mg, 0.03457 mmol, 1.0 equiv.) in DMF (2 mL) was added (2S,4R)-4-(difluoromethoxy)-N-(1H-pyrrolo[3,2-c]pyridin-2-ylmethyl)pyrrolidine-2-carboxamide hydrochloride (B, 11.99 mg, 0.03457 mmol, 1.0 equiv.), T3P (50 mass %) in ethyl acetate (31.00 μL, 0.0518 mmol, 1.5 equiv.), and DIPEA (18.10 μL, 0.104 mmol, 3.0 equiv.). The reaction mixture was stirred at room temperature for 30 minutes and then purified by directly by prep HPLC to give Compound 154 (4.125 mg, 0.007094 mmol, 100 mass %, 20.52% yield). LC/MS (ESI) m/z: 582 (M+H)⁺. ¹H NMR (400 MHz, DMSO-d6): δ 11.66 (s, 1H), 8.89-8.45 (m, 4H), 8.15 (s, 1H), 7.68 (d, J=8.1 Hz, 1H), 7.50 (q, J=9.5 Hz, 3H), 7.31-7.17 (m, 4H), 7.11 (dd, J=8.7, 4.7 Hz, 3H), 7.01 (s, 1H), 6.82 (s, 1H), 6.67-6.45 (m, 2H), 4.98 (s, 1H), 4.46 (d, J=8.6 Hz, 3H), 4.29-3.42 (m, 3H), 2.39-2.28 (m, 3H), 2.21-2.06 (m, 2H). RT (Method A): 1.50 min.

Compound 240 was prepared based on Scheme 45:

| Cmpd # | Reactant A | Reactant B | Reactant C | Characterization Data |
|---|---|---|---|---|
| 240 | 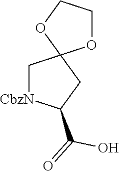 [SMILES: O=C(O)[C@@H]1CC2(CN1C(=O)OCc1ccccc1)OCCO2] | 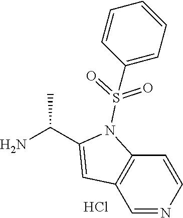 | 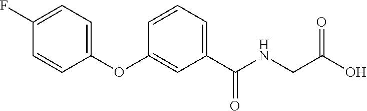 [SMILES: O=C(O)CNC(=O)c1cccc(Oc2ccc(F)cc2)c1] | ¹H NMR (400 MHz, DMSO-d6): δ 10.99 (s, 1H), 8.77 (t, J = 5.8 Hz, 1H), 8.62 (d, J = 10.7 Hz, 2H), 8.46 (d, J = 8.5 Hz, 1H), 8.19 (s, 1H), 7.86 (d, J = 5.6 Hz, 1H), 7.63 (d, J = 7.7 Hz, 1H), 7.52- 7.37 (m, 2H), 7.25-7.10 (m, 4H), 7.09-6.98 (m, 3H), 6.96 (d, J = 5.7 Hz, 1H), 6.37 (d, J = 16.8 Hz, 1H), 5.08 (h, J = 7.4 Hz, 1H), 4.32 (t, J = 8.1 Hz, 1H), 4.13 (dd, J = 16.7, 6.0 Hz, 1H), 3.98- 3.77 (m, 6H), 3.75 (d, J = 10.9 Hz, 1H), 3.57 (d, J = 10.8 Hz, 1H), 2.36-2.13 (m, 1H), 2.05 (dd, J = 12.9, 8.3 Hz, 1H), 1.38 (d, J = 7.0 Hz, 3H). LC/MS (ESI) m/z: 588 (M + H)⁺. RT (Method A): 1.29 min. |

^{a}Deprotection reaction in Step 2 was performed in MeOH using Pd/C instead of TFA in DCM.

### Scheme 46. Synthesis of (2S,3R)-N-((1H-pyrrolo[3,2-c]pyridin-2-yl)methyl)-3-hydroxy-1-((phenoxathiine-3-carbonyl)glycyl)pyrrolidine-2-carboxamide (Compound 158)

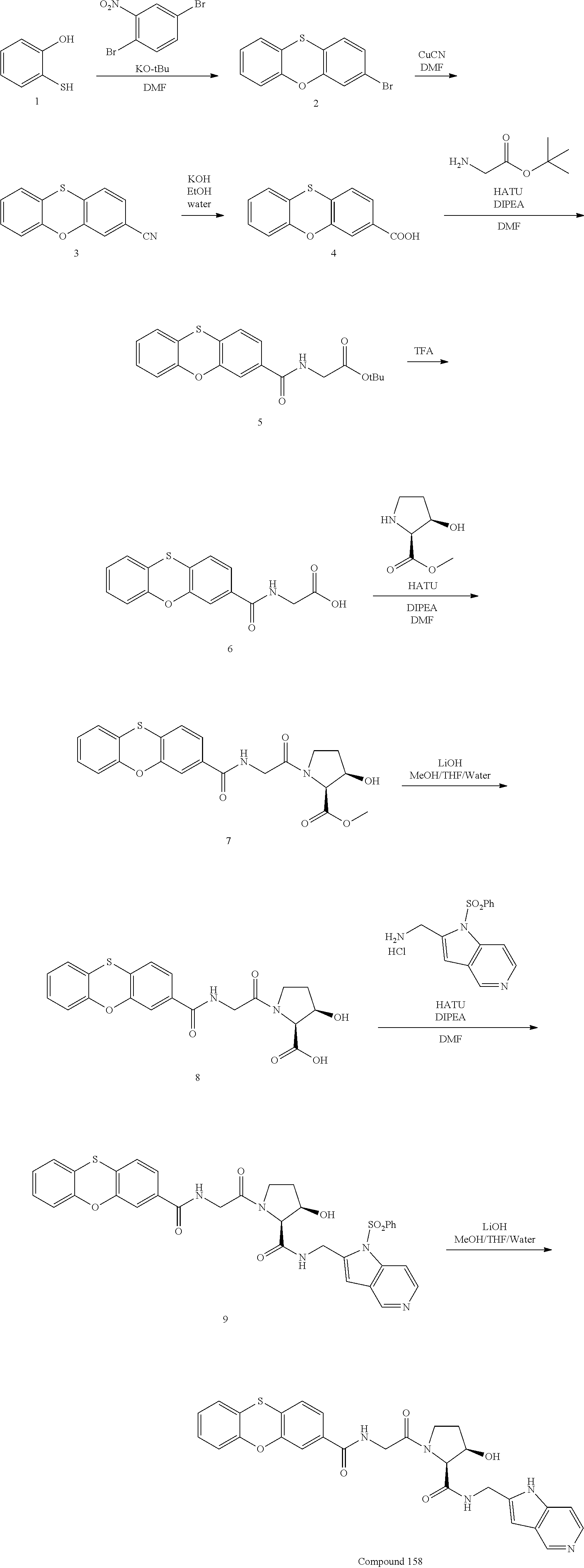

### Step 1: 3-Bromophenoxathinne (2)

In a sealed tube, potassium tert-butoxide (5.47 g, 48.7 mmol) is added to a solution of 2-sulfanylphenol (3 g, 23.8 mmol) in DMF (120 mL) at room temperature. After stirring for 30 minutes, 1,4-dibromo-2-nitrobenzene (7 g, 24.9 mmol) was added and the mixture stirred for another 20 minutes. The dark suspension was heated to 150° C. for 65 hours. After cooling down to room temperature, the reaction mixture was diluted with water (800 mL), stirred for 20 minutes. The yellow precipitated was filtered and re-dissolved in DCM, washed with saturated brine, dried over Na₂SO₄, filtered, and concentrated to give 3-bromophenoxathiine (5 g, 75.3% yield) as a yellow solid. No mass observed on LC/MS.

### Step 2: Phenoxathiine-3-carbonitrile (3)

A mixture of cuprous cyanide (150 mg, 1.6 mmol) and 3-bromophenoxathiine (0.23 g, 0.82 mmol) in DMF (2 mL) was heated to reflux for 15 hours. After cooling down to room temperature, the mixture was diluted with water, and the insoluble solid was removed by filtration. The filtrate was extracted with ethyl acetate twice. The combined organic layers were washed with saturated brine, dried over Na₂SO₄, filtered, and concentrated to give phenoxathiine-3-carbonitrile (90 mg, 48% yield) as a yellow solid. No mass observed on LC/MS.

### Step 3: Phenoxathiine-3-carboxylic acid (4)

To a solution of phenoxathiine-3-carbonitrile (90 mg, 0.4 mmol) in EtOD (0.8 g, 20 mmol) and water (5 mL) was added KOH (0.11 g, 2.0 mmol). The reaction mixture was heated to reflux for 5 hours. The mixture was concentrated under reduced pressure, diluted with water, and extracted with ethyl acetate twice. The combined organic layers were washed with brine, saturated brine, dried over Na₂SO₄, filtered, and concentrated to give phenoxathiine-3-carboxylic acid (100 mg, >theory) as an orange solid.

### Step 4: tert-Butyl (phenoxathiine-3-carbonyl)glycinate (5)

To a solution of phenoxathiine-3-carboxylic acid (100 mg, 0.41 mmol), tert-butyl 2-aminoacetate (100 mg, 0.82 mmol), and HATU (200 mg, 0.61 mmol) in DMF (1.1 mL) was added DIPEA (0.27 mL, 1.23 mmol) at room temperature. After 30 minutes, the reaction mixture was diluted with water and extracted with ethyl acetate for three times. The combined organic layers were washed with saturated brine, filtered, and concentrated under reduced pressure. The residue was purified on silica gel (0 to 80% ethyl acetate in heptane) to give tert-butyl 2-(phenoxathiine-3-carbonylamino)acetate (31 mg, 21.2% yield) as a yellow solid. LC/MS (ESI) m/z: 358 (M+H)⁺.

### Step 5: 2-(Phenoxathiine-3-carbonylamino)acetic acid (6)

To a solution of tert-butyl 2-(phenoxathiine-3-carbonylamino)acetate (31 mg, 0.087 mmol) in DCM (0.3 mL) was added TFA (0.12 mL, 1.6 mmol) at room temperature. The reaction mixture was stirred at room temperature for 18 hours. The volatiles were removed by concentration under reduced pressure. The residue was azeotroped with dichloromethane twice to give 2-(phenoxathiine-3-carbonylamino)acetic acid (26 mg, 99% yield) as a yellow solid. This material was used in next step without further purification. LC/MS (ESI) m/z: 302 (M+H)⁺.

### Step 6: Methyl (2S,3R)-3-hydroxy-1-[2-(phenoxathiine-3-carbonylamino)acetyl]pyrrolidine-2-carboxylate (7)

To a solution of 2-(phenoxathiine-3-carbonylamino)acetic acid (26 mg, 0.086 mmol), methyl (2S,3R)-3-hydroxypyrrolidine-2-carboxylate (30 mg, 0.20667 mmol), HATU (1.5 equiv., 0.13 mmol) in DMF (1 mL) was added DIPEA (0.05 mL, 0.26 mmol) at room temperature. The reaction mixture was stirred for 30 minutes, and the crude reaction mixture was directly purified on a C-18 column (0 to 100% acetonitrile in water) to give methyl (2S,3R)-3-hydroxy-1-[2-(phenoxathiine-3-carbonylamino)acetyl]pyrrolidine-2-carboxylate (60 mg, >theory) as a yellow wax. LC/MS (ESI) m/z=429 (M+H).

### Step 7: (2S,3R)-3-Hydroxy-1-((phenoxathiine-3-carbonyl)glycyl)pyrrolidine-2-carboxylic acid (8)

To a solution of methyl (2S,3R)-3-hydroxy-1-[2-(phenoxathiine-3-carbonylamino)acetyl]pyrrolidine-2-carboxylate (60 mg, 0.09 mmol) in MeOH (0.25 mL), water (0.25 mL) and THF (0.25 mL) was added LiOH·H₂O (43 mg, 0.1 mmol) at room temperature. The reaction mixture was stirred for 18 hours. The solvent was removed under reduced pressure, and azeotroped with methanol for three times to give (2S,3R)-3-hydroxy-1-[2-(phenoxathiine-3-carbonylamino)acetyl]pyrrolidine-2-carboxylic acid (40 mg, theory yield) as a yellow solid. LC/MS (ESI) m/z: 415 (M+H).

### Step 8: (2S,3R)-N-[[1-(Benzenesulfonyl)pyrrolo[3,2-c]pyridin-2-yl]methyl]-3-hydroxy-1-[2-(phenoxathiine-3-carbonylamino)acetyl]pyrrolidine-2-carboxamide (9)

To a solution of (2S,3R)-3-hydroxy-1-[2-(phenoxathiine-3-carbonylamino)acetyl]pyrrolidine-2-carboxylic acid (40 mg, 0.097 mmol), HATU (73 mg, 0.19 mmol), and [1-(benzenesulfonyl)pyrrolo[3,2-c]pyridin-2-yl]methanamine hydrochloride (45 mg, 0.1324 mmol) in DMF (2 mL) was added DIPEA (0.06 mL, 0.34 mmol) at 0° C. The reaction mixture was stirred at 0° C. for 30 minutes. The reaction mixture was directly purified on a C-18 column (0 to 35% acetonitrile in water) to give of (2S,3R)-N-[[1-(benzenesulfonyl)pyrrolo[3,2-c]pyridin-2-yl]methyl]-3-hydroxy-1-[2-(phenoxathiine-3-carbonylamino)acetyl]pyrrolidine-2-carboxamide (50 mg, 75.8% yield) as a yellow solid. LC/MS (ESI) m/z: 684 (M+H)⁺.

### Step 9: (2S,3R)-3-Hydroxy-1-[2-(phenoxathiine-3-carbonylamino)acetyl]-N-(1H-pyrrolo[3,2-c]pyridin-2-ylmethyl)pyrrolidine-2-carboxamide (Compound 158)

To a solution of (2S,3R)-N-[[1-(benzenesulfonyl)pyrrolo[3,2-c]pyridin-2-yl]methyl]-3-hydroxy-1-[2-(phenoxathiine-3-carbonylamino)acetyl]pyrrolidine-2-carboxamide (50 mg, 0.073 mmol) in water (0.25 mL), MeOH (0.25 mL), and THF (0.25 mL) was added LiOH·H₂O (40 mg, 0.095 mmol) at room temperature. The mixture was stirred for 18 hours. The mixture was concentrated under reduced pressure, and diluted with DMSO and water, filtered and purified on prep-HPLC to give Compound 158 (3.5 mg, 8.5% yield) as a white solid. ¹H-NMR (400 MHz, DMSO-d6): δ 11.13 (s, 1H), 8.82-8.54 (m, 2H), 8.40-8.26 (m, 1H), 8.13-7.91 (m, 1H), 7.71-7.49 (m, 2H), 7.46-7.08 (m, 6H), 6.45 (m, 1H), 4.59-4.29 (m, 2H), 4.26-3.96 (m, 2H), 3.81-3.52 (m, 2H), 2.10-1.86 (m, 2H). LC/MS (ESI) m/z=544 (M+H)⁺. RT (Method A): 1.27 imn.

### Scheme 47. Synthesis of (5-((R)-1-((1S,3S,5S)-5-methyl-2-((4-phenoxybenzoyl) glycyl)-2-azabicyclo[3.1.0]hexane-3-carboxamido)ethyl)thiophen-3-yl)boronic acid (Compound 163)

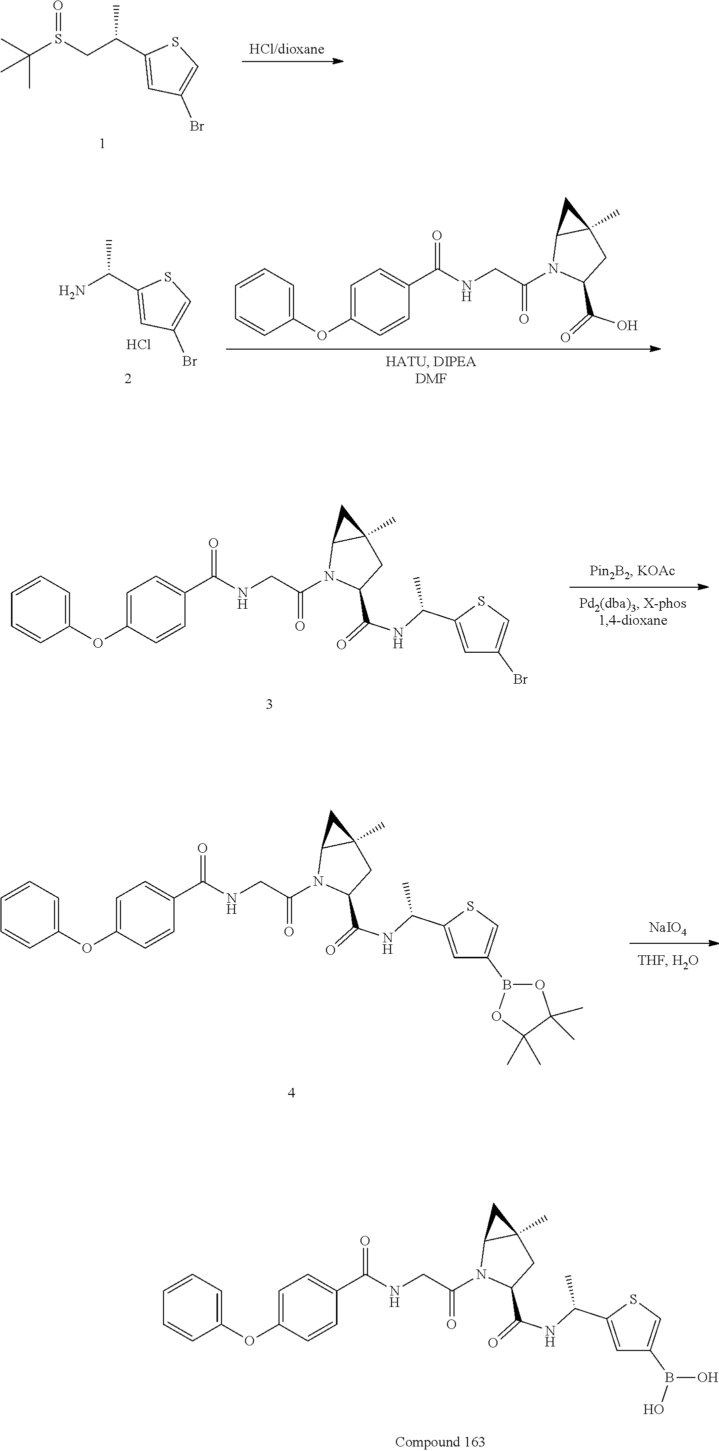

### Step 1: (R)-1-(4-Bromothiophen-2-yl)ethan-1-amine hydrochloride (2)

To a solution of N-((R)-1-(4-bromothiophen-2-yl)ethyl)-2-methylpropane-2-sulfinamide (50 mg, 0.16 mmol) in DCM (1 mL) was added HCl/1,4-dioxane (1 mL, 4M) and the reaction mixture was stirred at room temperature for 3 hours. The mixture was concentrated under reduced pressure to dryness to give the title compound (65 mg, crude) as a white solid, which was used directly in the next step without further purification. LC/MS (ESI) m/z: 241 (M+H)⁺.

### Step 2: (1S,3S,5S)-N-((R)-1-(4-bromothiophen-2-yl)ethyl)-5-methyl-2-((4-phenoxybenzoyl)glycyl)-2-azabicyclo[3.1.0]hexane-3-carboxamide (3)

To a mixture of (1S,3S,5S)-5-methyl-2-((4-phenoxybenzoyl)glycyl)-2-azabicyclo[3.1.0]hexane-3-carboxylic acid (65 mg, 0.16 mmol) and (R)-1-(4-bromothiophen-2-yl)ethan-1-amine hydrochloride (40 mg, 0.20 mmol) in DMF (2 mL) was added DIPEA (128 mg, 0.99 mmol) and HATU (76 mg, 0.20 mmol) at 0° C. and the mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=10:1) to give the title compound (60 mg, yield 62.7%) as a white solid. LC/MS (ESI) m/z: 582 (M+H)⁺.

### Step 3: (1S,3S,5S)-5-methyl-2-((4-phenoxybenzoyl)glycyl)-N-((R)-1-(4-(4,4,5,5-tetramethyl-1,3,2-dioxaborolan-2-yl)thiophen-2-yl)ethyl)-2-azabicyclo[3.1.0]hexane-3-carboxamide (4)

To a mixture of (1S,3S,5S)-N-((R)-1-(4-bromothiophen-2-yl)ethyl)-5-methyl-2-((4-phenoxybenzoyl)glycyl)-2-azabicyclo[3.1.0]hexane-3-carboxamide (60 mg, 0.10 mmol) and 4,4,4′,4′,5,5,5′,5′-octamethyl-2,2′-bi(1,3,2-dioxaborolane) (79 mg, 0.31 mmol) in 1,4-dioxane (2 mL) was added X-phos (10 mg, 0.02 mmol), Pd₂(dba)₃ (10 mg, 0.01 mmol) and KOAc (31 mg, 0.31 mmol), the mixture was degassed under N₂ atmosphere for three times and stirred at 80° C. overnight. The mixture was diluted with EtOAc, washed with water and brine, dried over anhydrous Na₂SO₄, filtered, and concentrated under reduced pressure to dryness. The residue was purified by prep-TLC (PE:EtOAc=11:1) to give the title compound (50 mg, yield 76.9%) as a white solid. LC/MS (ESI) m/z:630 (M+H)⁺.

### Step 4: (5-((R)-1-((1S,3S 5S)-5-methyl-2-((4-phenoxybenzoyl)glycyl)-2-azabicyclo[3.1.0]hexane-3-carboxamido)ethyl)thiophen-3-yl)boronic acid (Compound 163)

To a solution of (1S,3 S,5S)-5-methyl-2-((4-phenoxybenzoyl)glycyl)-N-((R)-1-(4-(4,4,5,5-tetramethyl-1,3,2-dioxaborolan-2-yl)thiophen-2-yl)ethyl)-2-azabicyclo[3.1.0]hexane-3-carboxamide (40 mg, 0.06 mmol) in THF/H₂O (2 mL, 1/1) was added NaIO₄ (28 mg, 0.13 mmol) and the mixture was stirred at room temperature for 2 hours. The mixture was quenched with Na₂S₂O₃ and concentrated under reduced pressure to dryness. The residue was purified by prep-HPLC to give the title compound (1.1 mug, yield 3.2%) as a white solid. ¹H NMR (400 MHz, CD₃OD): δ 7.83 (d, J=7.6 Hz, 2H), 7.64 (s, 1H), 7.41 (t, J=7.2 Hz, 2H), 7.22-7.16 (m, 2H), 7.06 (d, J=7.2 Hz, 2H), 6.99 (d, J=7.6 Hz, 2H), 5.24 (s, 1H), 4.78 (s, 1H), 4.39 (s, 1H), 4.27 (s, 1H), 3.37 (s, 1H), 2.37 (d, J=12.6 Hz, 1H), 2.13 (d, J=12.9 Hz, 1H), 1.57 (d, J=6.3 Hz, 3H), 1.29 (s, 3H), 1.26-1.24 (m, 1H), 0.82-0.76 (m, 1H). RT (Method A): 0.34 min.

### Scheme 48. Synthesis of (R)-N-((R)-1-(1H-pyrrolo[3,2-c]pyridin-2-yl)ethyl)-3,3-dimethyl-1-((phenoxathiine-3-carbonyl)glycyl)-1,3-azasilolidine-5-carboxamide (Compound 168)

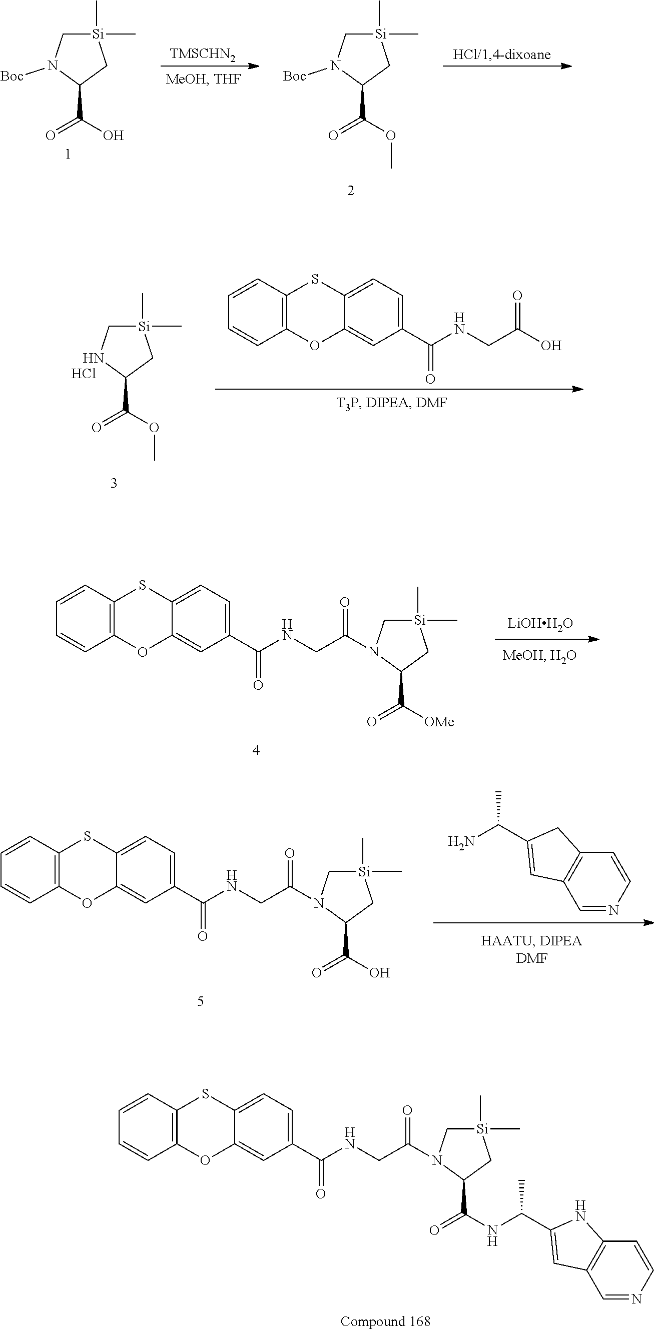

### Step 1: 1-(Tert-butyl) 5-methyl (R)-3,3-dimethyl-1,3-azasilolidine-1,5-dicarboxylate (2)

To a solution of (R)-1-(tert-butoxycarbonyl)-3,3-dimethyl-1,3-azasilolidine-5-carboxylic acid (50 mg, 0.19 mmol) in THF (2 mL) and MeOH (2 mL) was added TMSCHN₂ (0.2 mL, 0.40 mmol, 2M in hexane) at 0° C. and the mixture was stirred at 0° C. for 1 hour. The reaction mixture was concentrated under reduced pressure to dryness. The residue was purified by flash chromatography (silica gel, 0-25% EtOAc in PE) to give the title compound (45 mg, yield 86.5%) as a yellow oil. LC/MS (ESI) m/z: 274 (M+H)⁺.

### Step 2: Methyl (R)-3,3-dimethyl-1,3-azasilolidine-5-carboxylate hydrochloride (3)

A solution of 1-(tert-butyl) 5-methyl (R)-3,3-dimethyl-1,3-azasilolidine-1,5-dicarboxylate (45 mg, 0.16 mmol) in HCl/1,4-dioxane (3 mL, 4M) was stirred under N₂ atmosphere at room temperature for 2 hours. The reaction mixture was concentrated under reduced pressure to dryness to give the title compound (34 mg, yield 98.7%) as a white solid, which was used directly in the next reaction without further purification. LC/MS (ESI) m/z: 174 (M+H)⁺.

### Step 3: Methyl (R)-3,3-dimethyl-1-((phenoxathiine-3-carbonyl)glycyl)-1,3-azasilolidine-5-carboxylate (4)

To a mixture of methyl (R)-3,3-dimethyl-1,3-azasilolidine-5-carboxylate hydrochloride (34 mg, 0.16 mmol) and (phenoxathiine-3-carbonyl)glycine (53 mg, 0.18 mmol) in DMF (3 mL) was added DIPEA (116 mg, 0.90 mmol) and T₃P (153 mg, 0.24 mmol, 50% wt. in EtOAc) at 0° C. under N₂ atmosphere and the mixture was stirred at room temperature for 2 hours. The mixture was quenched with saturated aq. NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give methyl (R)-3,3-dimethyl-1-((phenoxathiine-3-carbonyl)glycyl)-1,3-azasilolidine-5-carboxylate (30 mg, yield 40.5%) as a white solid. LC/MS (ESI) (m/z): 457 (M+H)⁺.

### Step 4: (R)-3,3-dimethyl-1-((phenoxathiine-3-carbonyl)glycyl)-1,3-azasilolidine-5-carboxylic acid (5)

To a solution of methyl (R)-3,3-dimethyl-1-((phenoxathiine-3-carbonyl)glycyl)-1,3-azasilolidine-5-carboxylate (30 mg, 0.066 mmol) in THF/MeOH/H₂O (4 mL, 2/1/1) was added LiOH·H₂O (11 mg, 0.26 mmol) under N₂ atmosphere and the reaction mixture was stirred at room temperature for 2 hours. The mixture was acidified with IN aq. HCl to pH˜3 and extracted with EtOAc twice. The combined organic layers were dried over anhydrous Na₂SO₄, filtered, and concentrated under reduced pressure to give the title compound (29 mg, yield 99.76%) as a light-yellow oil, which was used directly in the next reaction without further purification. LC/MS (ESI) m/z: 443 (M+H)⁺.

### Step 5: (R)-N-((R)-1-(H-pyrrolo[3,2-c]pyridin-2-yl)ethyl)-3,3-dimethyl-1-((phenoxathiine-3-carbonyl)glycyl)-1,3-azasilolidine-5-carboxamide (Compound 168)

To a solution of (R)-3,3-dimethyl-1-((phenoxathiine-3-carbonyl)glycyl)-1,3-azasilolidine-5-carboxylic acid (22 mg, 0.050 nmol) in DMF (0.7 mL) was added (R)-1-(1H-pyrrolo[3,2-c]pyridin-2-yl)ethan-1-amine (15 mg, 0.049 mmol), DIPEA (38 mg, 0.29 mmol) and HATU (20 mg, 0.054 mmol) under N₂ atmosphere and the reaction mixture was stirred at room temperature for 10 minutes. The mixture was diluted with EtOAc, washed with water and brine, dried over anhydrous Na₂SO₄, filtered, and concentrated under reduced pressure to dryness. The residue was purified by prep-TLC (10% MeOH in DCM) and further purified by prep-HPLC to give Compound 168 (3 mg, yield 10.5%) as a white solid. ¹H NMR (400 MHz, CD₃OD): δ 8.69 (d, J=19.2 Hz, 1H), 7.91 (d, J=5.6 Hz, 1H), 7.64-7.53 (m, 2H), 7.27 (d, J=8.0 Hz, 1H), 7.17 (dd, J=17.5, 7.9 Hz, 3H), 7.08 (t, J=7.1 Hz, 1H), 7.03 (d, J=7.9 Hz, 1H), 6.51 (d, J=27.7 Hz, 1H), 5.25 (d, J=6.3 Hz, 1H), 4.81-4.78 (m, 1H), 4.43 (d, J=17.2 Hz, 1H), 4.23 (d, J=17.0 Hz, 1H), 3.00 (d, J=15.1 Hz, 2H), 1.60 (d, J=6.7 Hz, 2H), 1.36 (d, J=5.8 Hz, 3H), 0.32 (s, 6H). LC/MS (ESI) m/z: 586 (M+H)⁺. RT (Method A): 1.60 min.

### Scheme 49. Synthesis of (2S,4S)-N-((R)-1-(1H-pyrrolo[3,2-c]pyridin-2-yl)ethyl)-1-((phenoxathiine-3-carbonyl)glycyl)-4-phenylpyrrolidine-2-carboxamide (Compound 169)

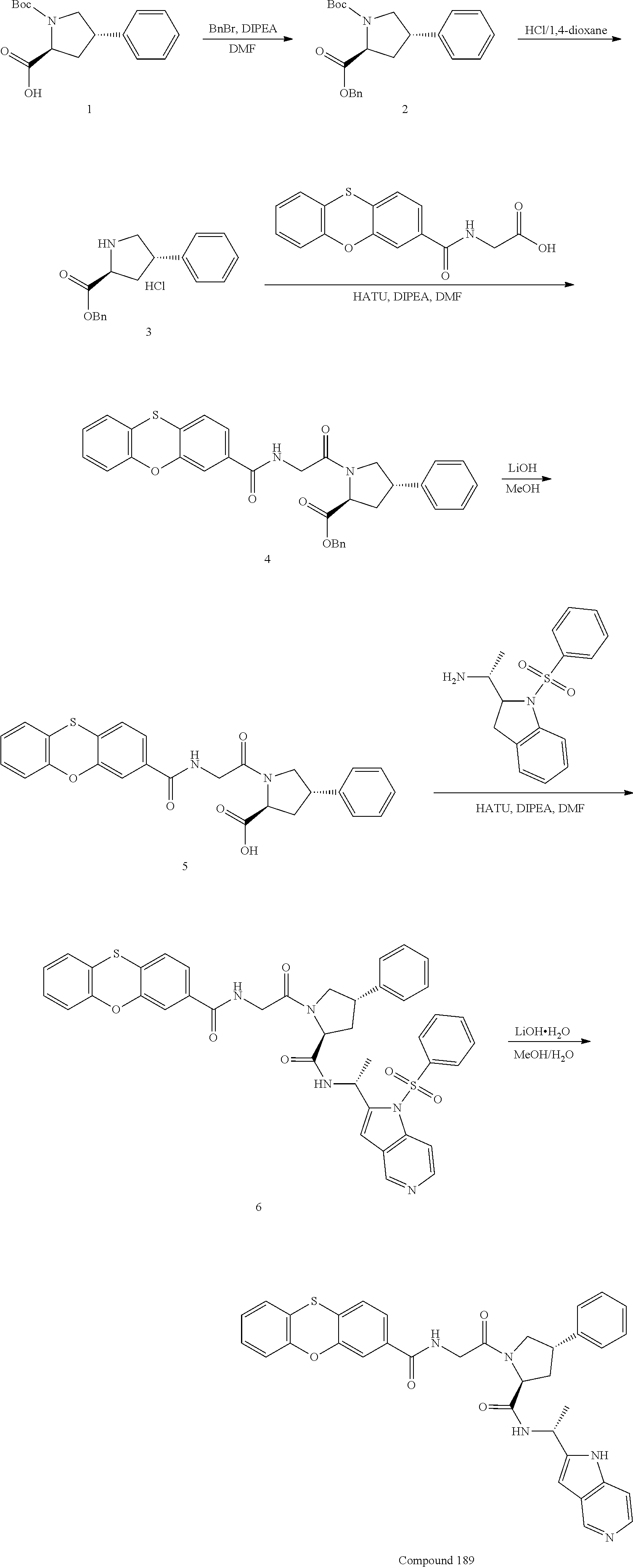

### Step 1: 2-Benzyl 1-(tert-butyl) (2S,4S)-4-phenylpyrrolidine-1,2-dicarboxylate (2)

To a solution of (2S,4S)-1-(tert-butoxycarbonyl)-4-phenylpyrrolidine-2-carboxylic acid (150 mg, 0.51 mmol) in DMF (2 mL) was added BnBr (260 mg, 1.5 mmol) and DIPEA (193 mg, 1.5 mmol) under N₂ atmosphere and the reaction mixture was stirred at room temperature for 30 minutes. The mixture was diluted with EtOAc, washed with water and brine, dried over anhydrous Na₂SO₄, filtered, and concentrated under reduced pressure to dryness. The residue was purified by flash chromatography (silica gel, 0-15% EtOAc in PE) to give the title compound (150 mg, yield 77.2%) as a colorless oil. LC/MS (ESI) m/z: 382 (M+H)⁺.

### Step 2: Benzyl (2S,4S)-4-phenylpyrrolidine-2-carboxylate hydrochloride (3)

To a solution of 2-benzyl 1-(tert-butyl) (2S,4S)-4-phenylpyrrolidine-1,2-dicarboxylate (150 mg, 0.39 mmol) in HCl/1,4-dioxane (2 mL, 4M) was stirred under N₂ atmosphere at room temperature for 2 hours. The reaction mixture was concentrated under reduced pressure to dryness to give the title compound (124 mg, yield 99.0%) as a yellow oil, which was used directly in the next step without further purification. LC/MS (ESI) m/z: 182 (M+H)⁺.

### Step 3: Benzyl (2S,4S)-1-((phenoxathiine-3-carbonyl)glycyl)-4-phenylpyrrolidine-2-carboxylate (4)

To a mixture of benzyl (2S,4S)-4-phenylpyrrolidine-2-carboxylate hydrochloride (47 mg, 0.14 mmol) and (phenoxathiine-3-carbonyl)glycine (50 mg, 0.16 mmol) in DMF (1.5 mL) was added DIPEA (93 mg, 0.72 mmol) and HA TU (61 mg, 0.16 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (5% MeOH in DCM) to give the title compound (60 mg, yield 73.4%) as a white solid. LC/MS (ESI) m/z: 565 (M+H)⁺.

### Step 4: (2S,4S)-1-((phenoxathiine-3-carbonyl)glycyl)-4-phenylpyrrolidine-2-carboxylic acid (5)

To a solution of benzyl (2S,4S)-1-((phenoxathiine-3-carbonyl)glycyl)-4-phenylpyrrolidine-2-carboxylate (60 mg, 0.11 mmol) in MeOH/H₂O (3 mL, 1/1) was added LiOH·H₂O (12 mg, 0.30 mmol) under N₂ atmosphere and the reaction mixture was stirred at room temperature for 2 hours. The mixture was acidified with IN aq. HCl to pH˜3 and extracted with EtOAc twice. The combined organic layers were washed with brine, dried over anhydrous Na₂SO₄, filtered, and concentrated under reduced pressure to dryness to give the title compound (40 mg, yield 79.6%) as a white solid, which was used directly in the next step without further purification. LC/MS (ESI) m/z: 475 (M+H)⁺.

### Step 5: (2S,4S)-1-((phenoxathiine-3-carbonyl)glycyl)-4-phenyl-N-((R)-1-(1-(phenylsulfonyl)-1H-pyrrolo[3,2-c]pyridin-2-yl)ethyl)pyrrolidine-2-carboxamide (6)

To a mixture of (2S,4S)-1-((phenoxathiine-3-carbonyl)glycyl)-4-phenylpyrrolidine-2-carboxylic acid (40 mg, 0.084 mmol) and (R)-1-(1-(phenylsulfonyl)-1H-pyrrolo[3,2-c]pyridin-2-yl)ethan-1-amine in DMF (0.8 mL) was added DIPEA (54 mg, 0.42 mmol) and HATU (38 mg, 0.10 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (8% MeOII in DCM) to give the title compound (20 mg, yield 31.4%) as a white solid. LC/MS (ESI) m/z: 758 (M+H)⁺.

### Step 5: (2S,4S)-N-((R)-1-(1H-pyrrolo[3,2-c]pyridin-2-yl)ethyl)-1-((phenoxathiine-3-carbonyl)glycyl)-4-phenylpyrrolidine-2-carboxamide (Compound 169)

To a solution of (2S,4S)-1-((phenoxathiine-3-carbonyl)glycyl)-4-phenyl-N-((R)-1-(1-(phenylsulfonyl)-1H-pyrrolo[3,2-c]pyridin-2-yl)ethyl)pyrrolidine-2-carboxamide (20 mg, 0.15 mmol) in MeOH/H₂O (6 mL, 1/1) was added LiOH·H₂O (12 mg, 0.30 mmol) and the mixture was stirred at room temperature for 2 hours. The mixture was acidified with IN aq. HCl to pH˜3 and extracted with EtOAc twice. The combined organic layers were washed with brine, dried over anhydrous Na₂SO₄, filtered, concentrated under reduced pressure to dryness. The residue was purified by prep-HPLC to give Compound 169 (4.0 mg, yield 24.5%) as a white solid. ¹H NMR (400 MHz, CD₃OD): δ 8.67 (d, J=15.3 Hz, 1H), 8.00 (t, J=17.9 Hz, 1H), 7.60-7.48 (m, 2H), 7.33 (s, 4H), 7.19 (dd, J=32.7, 12.9 Hz, 5H), 7.05 (dd, J=22.2, 7.3 Hz, 2H), 6.56 (d, J=22.7 Hz, 1H), 5.31 (d, J=6.6 Hz, 1H), 4.66 (s, 1H), 4.32-4.07 (m, 3H), 3.94-3.66 (m, 2H), 2.61-2.37 (m, 2H), 1.63 (dd, J=24.4, 6.5 Hz, 3H). LC/MS (ESI) m/z: 618 (M+H)⁺. RT (Method A): 1.73 min.

### Scheme 50. Synthesis of (2S,4R)-N-((R)-1-(1H-pyrrolo[3,2-c]pyridin-2-yl)ethyl)-4-(cyclopropylsulfonyl)-1-((phenoxathiine-3-carbonyl)glycyl)pyrrolidine-2-carboxamide (Compound 174)

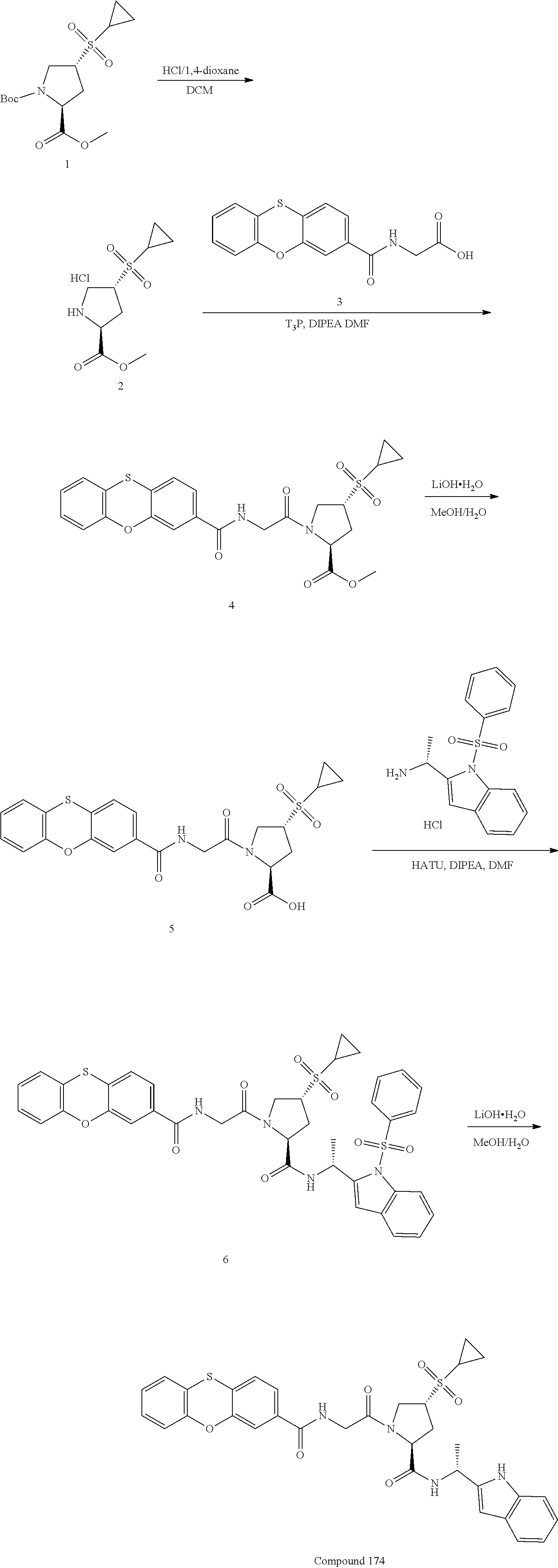

### Step 1: Methyl (2S,4R)-4-(cyclopropylsulfonyl)pyrrolidine-2-carboxylate hydrochloride (2)

A solution of 1-(tert-butyl) 2-methyl (2S,4R)-4-(cyclopropylsulfonyl)pyrrolidine-1,2-dicarboxylate (100 mg, 0.30 mmol) in HCl/1,4-dioxane (2 mL, 4M) was stirred under N₂ atmosphere at room temperature for 2 hours. The reaction mixture was concentrated under reduced pressure to dryness to give the title compound (68 mg, yield 97.2%) as a yellow solid, which was used directly in the next step without further purification. LC/MS (ESI) m/z: 234 (M+H)⁺.

### Step 2: Methyl (2S,4R)-4-(cyclopropylsulfonyl)-1-((phenoxathiine-3-carbonyl)glycyl) pyrrolidine-2-carboxylate (3)

To a mixture of (phenoxathiine-3-carbonyl)glycine (40 mg, 0.13 mmol) and methyl (2S,4R)-4-(cyclopropylsulfonyl)pyrrolidine-2-carboxylate hydrochloride (46 mg, 0.20 mmol) in DMF (3 mL) was added DIPEA (103 mug, 0.78 mmol) and T₃P (127 mg, 0.20 mmol, 50% wt. in EtOAc) under N₂ atmosphere at 0° C. and the mixture was stirred at room temperature for 2 hours. The mixture was quenched with saturated aq. NaH CO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-60% EtOAc in PE) to give the title compound (50 mg, yield 72.9%) as a white solid. LC/MS (ESI) (m/z): 517 (M+H)⁺.

### Step 3: (2S,4R)-4-(cyclopropylsulfonyl)-1-((phenoxathiine-3-carbonyl)glycyl) pyrrolidine-2-carboxylic acid (4)

To a solution of methyl (2S,4R)-4-(cyclopropylsulfonyl)-1-((phenoxathiine-3-carbonyl) glycyl)pyrrolidine-2-carboxylate (50 mg, 0.10 mmol) in MeOH (4 mL) and water (1 mL) was added LIOH·H₂O (12 mg, 0.30 mmol) and the mixture was stirred at room temperature for 2 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (40 mg, yield 82.3%) as a white solid, which was used directly in the next step without further purification. LC/MS (ESI) m/z: 503 (M+H)⁺.

### Step 4: (2S,4R)-4-(cyclopropylsulfonyl)-1-((phenoxathiine-3-carbonyl)glycyl)-N-((R)-1-(1-(phenylsulfonyl)-1H-pyrrolo[3,2-c]pyridin-2-yl)ethyl)pyrrolidine-2-carboxamide (5)

To a mixture of (2S,4R)-4-(cyclopropylsulfonyl)-1-((phenoxathiine-3-carbonyl)glycyl) pyrrolidine-2-carboxylic acid (40 mg, 0.08 mmol) and (R)-1-(1-(phenylsulfonyl)-1H-pyrrolo[3,2-c]pyridin-2-yl)ethan-1-amine hydrochloride (36 mg, 0.12 mmol) in DMF (2 mL) was added DIPEA (62 mg, 0.48 mmol) and HATU (36 mg, 0.10 mmol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=10:1) to give the title compound (45 mg, yield 71.9%) as a yellow solid. LC/MS (ESI) m/z: 786 (M+H)⁺.

### Step 5: (2S,4R)-N-((R)-1-(1H-pyrrolo[3,2-c]pyridin-2-yl)ethyl)-4-(cyclopropylsulfonyl)-1-((phenoxathiine-3-carbonyl)glycyl)pyrrolidine-2-carboxamide (Compound 174)

To a solution of (2S,4R)-4-(cyclopropylsulfonyl)-1-((phenoxathiine-3-carbonyl)glycyl)-N-((R)-1-(1-(phenylsulfonyl)-1H-pyrrolo[3,2-c]pyridin-2-yl)ethyl)pyrrolidine-2-carboxamide (45 mg, 0.06 mmol) in MeOH (2 mL) and water (1 mL) was added LiOH·H₂O (7 mg, 0.18 mmol) and the mixture was stirred at room temperature for 2 hours. The mixture was extracted with EtOAc twice. The combined organic layers were washed with brine, dried over anhydrous Na₂SO₄, filtered, and concentrated under reduced pressure to dryness. The residue was purified by prep-HPLC to give Compound 174 (8 mg, yield 21.6%) as a white solid. ¹H NMR (400 MHz, CD₃OD): δ 8.89 (d, J=18.0 Hz, 1H), 8.43 (s, 1H), 8.06 (d, J=6.8 Hz, 1H), 7.53 (d, J=7.5 Hz, 3H), 7.18 (d, J=19.8 Hz, 3H), 7.05 (d, J=29.8 Hz, 2H), 6.83 (s, 1H), 5.32 (s, 1H), 4.71 (s, 1H), 4.25 (d, J=29.4 Hz, 5H), 4.14-3.98 (m, 1H), 2.78 (s, 1H), 2.48 (s, 1H), 1.71-1.61 (m, 3H), 1.18 (d, J=18.4 Hz, 4H). LC/MS (ESI) m/z: 646 (M+H)⁺. RT (Method A): 1.39 min.

The following compounds were prepared based on Scheme 50:

| Cmpd # | Reactant A | Reactant B | Reactant C | Characterization Data |
|---|---|---|---|---|
| 176 | 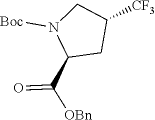 [SMILES: CC(C)(C)OC(=O)N1C[C@H](C(F)(F)F)C[C@H]1C(=O)OCc1ccccc1] | 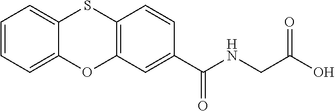 [SMILES: O=C(O)CNC(=O)c1ccc2c(c1)Oc1ccccc1S2] | 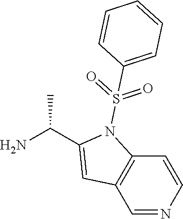 | ¹H NMR (400 MHz, CD₃OD): δ 8.67 (d, J - 16.6 Hz, 1H), 7.98 (t, J - 21.4 Hz, 1H), 7.54 (dd, J = 23.9, 15.7 Hz, 2H), 7.26-7.14 (m, 4H), 7.06 (dd, J = 21.6, 7.4 Hz, 2H), 6.56 (d, J = 26.2 Hz, 1H), 5.28 (d, J = 7.1 Hz, 1H), 4.63 (s, 1H), 4.16 (ddd, J = 31.9, 26.5, 13.1 Hz, 3H), 3.88 (d, J = 6.8 Hz, 1H), 3.46 (d, J = 7.8 Hz, 1H), 2.40 (dd, J = 25.8, 18.9 Hz., 2H), 1.63 (dd, J = 22.9, 6.3 Hz, 3H). L.C/MS (ESI) m/z: 610 (M + H)⁺. RT (Method A): 1.55 min. |
| 178^{a} | 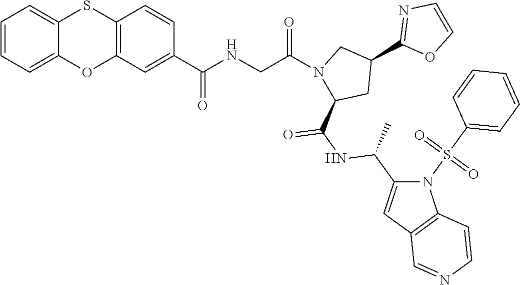 | | | ¹H NMR (400 MHz, CD₃OD): δ 8.67 (d, J = 15.5 Hz, 1H), 7.93-7.85 (m, 2H), 7.59 (d, J = 10.9 Hz, 2H), 7.26 (d, J = 7.9 Hz, 1H), 7.21-7.15 (m, 2H), 7.10 (s, 3H), 7.03 (d, J - 7.2 Hz, 1H), 6.51 (s, 1H), 5.28 (d, J - 7.1 Hz, 1H), 4.55 (t, J - 8.2 Hz, 1H), 4.42 (d, J = 16.4 Hz, 1H), 4.28 (t, J = 8.7 Hz, 1H), 4.15 (d, J = 16.0 Hz, 1H), 3.96 (t, J = 9.9 Hz, 1H), 3.82 (d, J = 8.1 Hz, 1H), 2.76 (s, 1H), 2.36 (d, J = 11.1 Hz, 1H), 1.60 (d, J = 7.0 Hz, 3H). LC/MS (ESI) m/z: 609 (M + H)⁺. RT (Method A): 1.35 min. |
| 180^{b} | 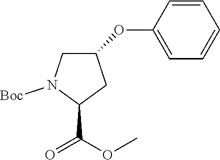 | 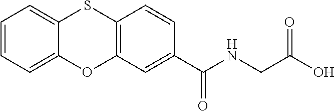 [SMILES: O=C(O)CNC(=O)c1ccc2c(c1)Oc1ccccc1S2] | 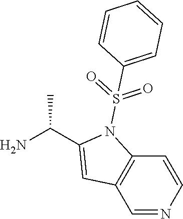 | ¹H NMR (400 MHz, DMSO-d6): δ 11.10 (s, 1H), 8.86 (s, 1H), 8.73-8.64 (m, 3H), 8.54 (s, 1H), 7.87 (d, J = 5.6 Hz, 1H), 7.64 (s, 2H), 7.47-7.38 (m, 1H), 7.36-7.24 (m, 6H), 7.13 (d, J = 8.1 Hz, 3H), 7.00 (d, J = 7.7 Hz, 3H), 6.95 (d, J = 5.6 Hz, 2H), 6.40 (s, 1H), 5.16 (dd, J = 20.0, 12.0 Hz, 2H), 4.48 (t, J = 8.4 Hz, 1H), 4.25 (dd, J = 17.1, 6.1 Hz, 1H), 4.06 (d, J = 6.7 Hz, 1H), 4.01 (d, J = 5.4 Hz, 1H), 3.90 (d, J = 11.4 Hz, 2H), 2.03-1.95 (m, 2H), 1.47 (d, J = 7.0 Hz, 4H). LC/MS (ESI) m/z: 634 (M + H)⁺. RT (Method A): 1.74 min. |

| | | |
|---|---|---|
| 183^{a} | 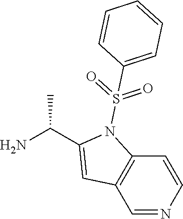 | ¹H NMR (400 MHz, CD₃OD): δ 8.78 (dd, J = 78.1, 10.4 Hz, 1H), 8.45 (s, 1H), 8.05 (dd, J = 14.3, 8.4 Hz, 2H), 7.76 (d, J = 9.5 Hz, IH), 7.53-7.48 (m, 1H), 7.38-7.33 (m, 1H), 7.24-7.15 (m, 2H), 7.09 (t, J = 6.2 Hz, 2H), 7.02 (d, J = 7.5 Hz, 1H), 6.75 (dd, J = 42.7, 16.3 Hz, 1H), 5.34 (s, 1H), 4.62 (dd, J = 17.3, 6.3 Hz, 2H), 4.52 (d, J = 10.0 Hz, 1H), 4.29-4.19 (m, 1H), 4.06 (dd, J = 36.3, 18.2 Hz, 2H), 3.66 (dd, J = 22.6, 10.8 Hz, 1H), 3.10-2.98 (m, 1H), 2.21 (d, J = 6.9 Hz, 1H), 1.69 (dd, J = 18.1, 10.7 Hz, 3H). LC/MS (ESI) m/z: 623 (M + H)⁺. RT (Method A): 1.84 min. |

^{a}Step 5 only.
^{b}HATU used in place of T₃P in Step 2.

### Scheme 51. Synthesis of (1S,3S,5S)-N-((2-amino-11H-benzo[d]imidazol-6-yl)methyl)-5-methyl-2-((phenoxathiine-3-carbonyl)glycyl)-2-azabicyclo[3.1.0]hexane-3-carboxamide (Compound 181)

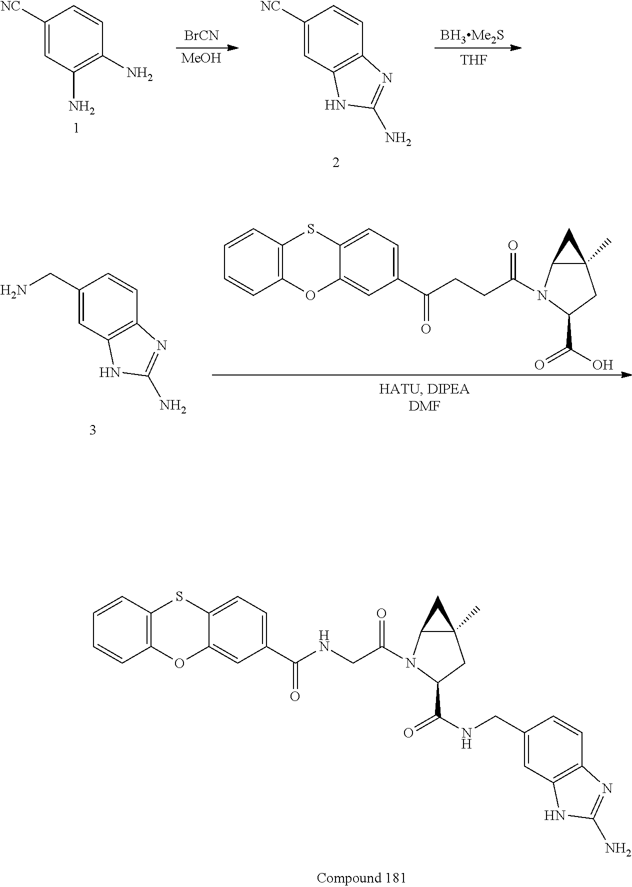

### Step 1: 2-Amino-JH-benzo[d]imidazole-6-carbonitrile (2)

To a mixture of 3,4-diaminobenzonitrile (550 mg, 4.14 mmol) and BrCN (525 mg, 4.96 mmol) in MeOH (8 ml) under N₂ atmosphere, the mixture was degassed under N₂ atmosphere for three times and stirred at 50° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give the title compound (320 mg, yield 49.1%) as a brown oil. LC/MS (ESI) m/z: 159 (M+H)⁺.

### Step 2: 6-(Aminomethyl)-1H-benzo[d]imidazol-2-amine (3)

To a solution of 2-amino-1H-benzo[d]imidazole-6-carbonitrile (170 mg, 1.08 mmol) in THF (3 mL) was added BH₃·Me₂S (0.65 mL, 6.48 mmol) under N₂ atmosphere and the mixture was stirred at 70° C. for 2 hours. The mixture was quenched with MeOH and diluted with EtOAc. The mixture was washed with saturated aq. NaHCO₃ solution and brine, dried over anhydrous Na₂SO₄, filtered, and concentrated under reduced pressure to dryness to give the title compound (100 mg, yield 57.4%) as a white solid. LC/MS (ESI) m/z: 163 (M+H)⁺.

### Step 3: (1S,3S,5S)-N-((2-amino-1H-benzo[d]imidazol-6-yl)methyl)-5-methyl-2-((phenoxathiine-3-carbonyl)glycyl)-2-azabicyclo[3.1.0]hexane-3-carboxamide (Compound 181)

To a mixture of (1S,3S,5S)-5-methyl-2-((phenoxathiine-3-carbonyl)glycyl)-2-azabicyclo [3.1.0]hexane-3-carboxylic acid (30 mg, 0.07 mmol) and 6-(aminomethyl)-1H-benzo[d]imidazol-2-amine (23 mg, 0.14 mmol) in DMF (3 mL) was added DIPEA (46 mg, 0.35 mmol) and HATU (35 mg, 0.09 mmol) at 0° C. under N₂ atmosphere and the mixture was stirred at room temperature for 1 hour. The mixture was diluted with EtOAc, washed with brine, dried over anhydrous Na₂SO₄, filtered, and concentrated under reduced pressure to dryness. The residue was purified by prep-TLC (DCM:MeOH=10:1) and further purified by prep-HPLC to give Compound 181 (1.1 mg, yield 2.7%) as a white solid. ¹H NMR (400 MHz, CD₃OD): δ 7.43 (d, J=7.0 Hz, 1H), 7.32 (s, 1H), 7.24 (s, 1H), 7.21-7.13 (m, 4H), 7.09 (d, J=6.1 Hz, 1H), 7.01 (d, J=8.0 Hz, 1H), 4.90 (s, 4H), 4.57 (s, 2H), 4.48 (s, 2H), 4.33 (s, 2H), 3.43 (s, 2H), 2.52-2.43 (m, 1H), 2.20 (d, J=12.8 Hz, 1H), 1.31 (s, 3H), 1.22-1.18 (m, 1H), 0.87 (dd, J=14.8, 3.8 Hz, 1H). LC/MS (ESI) m/z: 569 (M+H)⁺. RT (Method A): 1.50 min.

### Scheme 52. Synthesis of (2S,4R)-4-((1H-1,2,3-triazol-1-yl)methyl)-N-((R)-1-(1H-pyrrolo[3,2-c]pyridin-2-yl)ethyl)-1-((phenoxathiine-3-carbonyl)glycyl)pyrrolidine-2-carboxamide (Compound 182)

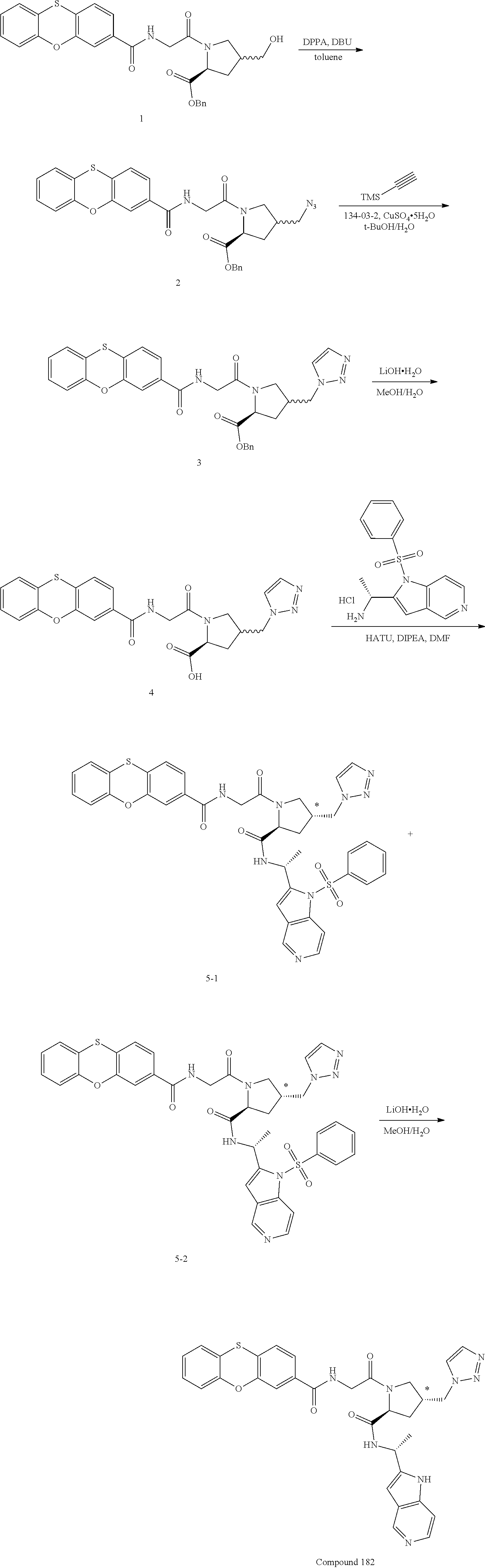

### Step 1: Benzyl (2S)-4-(azidomethyl)-1-((phenoxathiine-3-carbonyl)glycyl) pyrrolidine-2-carboxylate (2)

To a mixture of benzyl (2S)-4-(hydroxymethyl)-1-((phenoxathiine-3-carbonyl)glycyl) pyrrolidine-2-carboxylate (300 mg, 0.58 mmol) and DBU (264 mg, 1.73 mmol) in toluene. (3 mL) was added DPPA (319 mg, 1.16 mmol) and the mixture was stirred at 100° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (110 mg, yield 34.9%) as a yellow oil. LC/MS (ESI) (m/z): 544 (M+H)⁺.

### Step 2: Benzyl (2S)-4-((1H-1,2,3-triazol-1-yl)methyl)-1-((phenoxathiine-3-carbonyl) glycyl)pyrrolidine-2-carboxylate (3)

To a mixture of benzyl (2S)-4-(azidomethyl)-1-((phenoxathiine-3-carbonyl)glycyl) pyrrolidine-2-carboxylate (110 mg, 0.203 mmol) and ethynyltrimethylsilane (264 mg, 1.73 mmol) in t-BuOH (0.6 mL) and water (1.2 mL) was added CuSO₄·5H₂O (109 mg, 0.406 mmol) and sodium ascorbate (225 mg, 1.13 mmol) and the mixture was stirred at 60° C. for 3 hours. The mixture was diluted with EtOAc, washed with brine, dried over anhydrous Na₂SO₄, filtered, and concentrated under reduced pressure to dryness. The residue was purified by flash chromatography (silica gel, 0-50% EtOAc in PE) to give the title compound (40 mg, yield 34.8%) as a yellow oil. LC/MS (ESI) (m/z): 570 (M+H)⁺.

### Step 3: (2S)-4-((1H-1,2,3-triazol-1-yl)methyl)-1-((phenoxathiine-3-carbonyl)glycyl) pyrrolidine-2-carboxylic acid (4)

To a solution of (2S,4R)-4-((1H-1,2,3-triazol-1-yl)methyl)-N-((R)-1-(1H-pyrrolo[3,2-c]pyridin-2-yl)ethyl)-1-((phenoxathiine-3-carbonyl)glycyl)pyrrolidine-2-carboxamide (40 mg, 0.07 mmol) in MeOH (1 mL) and water (0.25 mL) was added LiOH·H₂O (6 mg, 0.14 mmol) and the mixture was stirred at room temperature for 2 hours. The mixture was acidified with IN aq. HCl to pH˜3 and extracted with EtOAc twice. The combined organic layers were washed with brine, dried over anhydrous Na₂SO₄, filtered, and concentrated under reduced pressure to dryness to give the title compound (28 mg, yield 84.8%) as a yellow oil, which was used directly in the next step without further purification. LC/MS (ESI) m/z: 480(M+H)⁺.

### Step 4: (2S,4R*)-4-((1H-1,2,3-triazol-1-yl)methyl)-1-((phenoxathiine-3-carbonyl) glycyl)-N-((R)-1-(1-(phenylsulfonyl)-1H-pyrrolo[3,2-c]pyridin-2-yl)ethyl)pyrrolidine-2-carboxamide (5-1) and (2S,4S*)-4-((1H-1,2,3-triazol-1-yl)methyl)-1-((phenoxathine-3-carbonyl)glycyl)-N-((R)-1-(1-(phenylsulfonyl)-1H-pyrrolo[3,2-c]pyridin-2yl)ethyl)pyrrolidine-2-carboxamide (5-2)

To a mixture of (2S)-4-((1H-1,2,3-triazol-1-yl)methyl)-1-((phenoxathiine-3-carbonyl) glycyl)pyrrolidine-2-carboxylic acid (28 mg, 0.058 mol) and (R)-1-(1-(phenylsulfonyl)-1H-pyrrolo[3,2-c]pyridin-2-yl)ethan-1-amine hydrochloride (26 mg, 0.087 mmol) in DMF (1 mL) was added DIPEA (37 mg, 0.29 mmol) and HATU (33 mg, 0.087 mol) under N₂ atmosphere and the reaction mixture was stirred at room temperature for 2 hours. The mixture was diluted with EtOAc, washed with brine, dried over anhydrous Na₂SO₄ filtered, and concentrated under reduced pressure to dryness. The residue was purified by prep-TLC (DCM:MeOH=20:1) to give (2S,4R*)-4-((1H-1,2,3-triazol-1-yl)methyl)-1-((phenoxathiine-3-carbonyl)glycyl)-N-((R)-1-(1-(phenylsulfonyl)-1H-pyrrolo[3,2-c]pyridin-2-yl)ethyl)pyrrolidine-2-carboxamide (23 mg, yield 52.3%) as a brown oil and (2S,4S*)-4-((III-1,2,3-triazol-1-yl)methyl)-1-((phenoxathiine-3-carbonyl)glycyl)-N-((R)-1-(1-(phenylsulfonyl)-1H-pyrrolo[3,2-c]pyridin-2-yl)ethyl) pyrrolidine-2-carboxamide (15 mg, yield 34.1%) as a brown oil. LC/MS (ESI) m/z: 763 (M+H)⁺.

### Step 5: (2S,4R*)-4-((1H-1,2,3-triazol-1-yl)methyl)-N-((R)-1-(1H-pyrrolo[3,2-c]pyridin-2-yl)ethyl)-1-((phenoxathiine-3-carbonyl)glycyl)pyrrolidine-2-carboxamide (Compound 182)

To a solution of (2S,4R*)-4-((1H-1,2,3-triazol-1-yl)methyl)-1-((phenoxathiine-3-carbonyl)glycyl)-N-((R)-1-(1-(phenylsulfonyl)-1H-pyrrolo[3,2-c]pyridin-2-yl)ethyl) pyrrolidine-2-carboxamide (23 mg, 0.03 mol) in MeOH (1 mL) and water (0.5 mL) was added LiOH·H₂O (3 mg, 0.06 mol) and the mixture was stirred at room temperature for 2 hours. The mixture was concentrated under reduced pressure to dryness. The residue was purified by prep-HPLC to give Compound 182 (1.4 mg, yield 7.5%) as a white solid. ¹H NMR (400 MHz, CD-OD): δ 8.92 (s, 1H), 8.35 (s, 1H), 8.13-8.01 (m, 2H), 7.78 (s, 1H), 7.53 (s, 2H), 7.21 (dd, J=26.9, 18.0 Hz, 3H), 7.06 (d, J=19.6 Hz, 2H), 6.86 (s, 1H), 5.30 (s, 1H), 4.65 (s, 1H), 4.52 (d, J=47.5 Hz, 3H), 4.22 (dd, J=34.7, 15.1 Hz, 2H), 3.90 (s, 1H), 3.57 (s, 1H), 3.08 (d, J=45.2 Hz, 1H), 2.15 (s, 1H), 1.63 (s, 3H). LC/MS (ESI) m/z: 623 (M+H)⁺. RT (Method A): 1.28 min.

### Scheme 53. Synthesis of (2S)-N-((R)-1-(1H-pyrrolo[3,2-c]pyridin-2-yl)ethyl)-1-((phenoxathiine-3-carbonyl)glycyl)-4-(piperidin-1-ylmethyl)pyrrolidine-2-carboxamide (Compound 185)

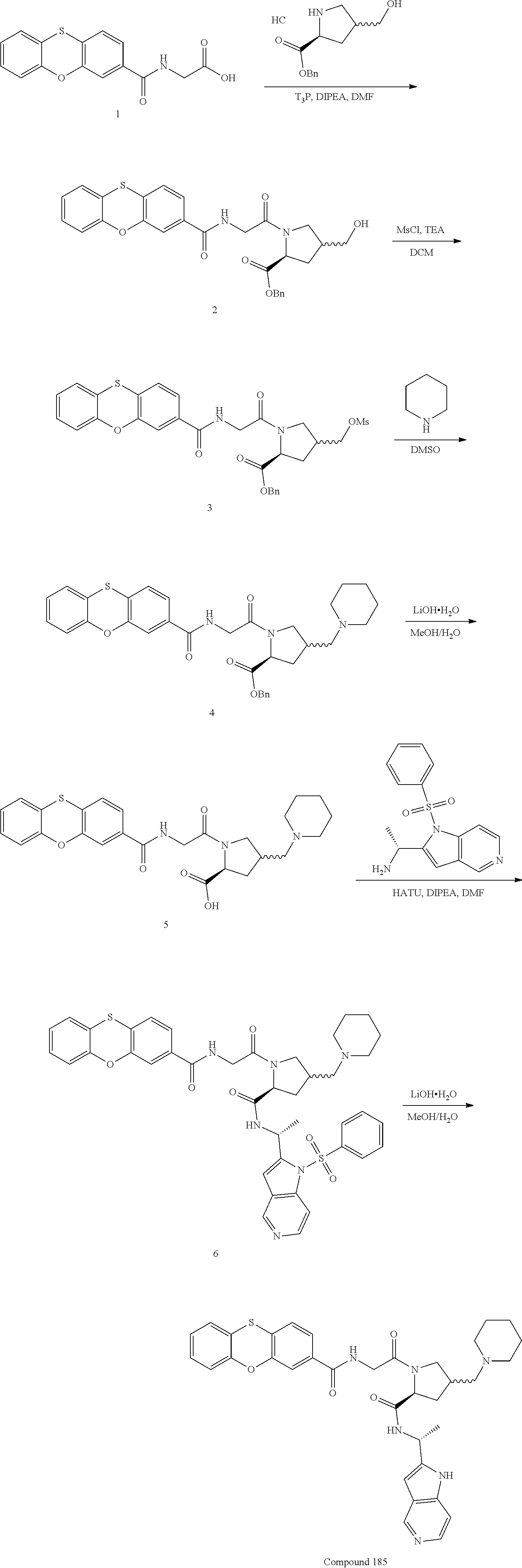

### Step 1: Benzyl (2S)-4-(hydroxymethyl)-1-((phenoxathiine-3-carbonyl)glycyl) pyrrolidine-2-carboxylate (2)

To a mixture of (phenoxathiine-3-carbonyl)glycine (600 mg, 1.99 mmol) and benzyl (2S)-4-(hydroxymethyl)pyrrolidine-2-carboxylate hydrochloride (562 mg, 2.39 mmol) in DMF (6 mL) was added DIPEA (1.29 g, 9.95 mmol) and T₃P (1.9 g, 2.99 mmol, 50% wt. in EtOAc) under N₂ atmosphere at 0° C. and the mixture was stirred at room temperature for 2 hours. The mixture was quenched with saturated aq. NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-60% EtOAc in PE) to give the title compound (828 mg, yield 80.2%) as a colorless oil. LC/MS (ESI) (m/z): 519 (M+H)⁺.

### Step 2: Benzyl (2S)-4-(((methylsulfonyl)oxy)methyl)-1-((phenoxathiine-3-carbonyl) glycol)pyrrolidine-2-carboxylate (3)

To a mixture of benzyl (2S)-4-(hydroxymethyl)-1-((phenoxathiine-3-carbonyl)glycyl) pyrrolidine-2-carboxylate (570 mg, 1.1 mmol) and TEA (333 mg, 3.3 mmol) in DCM (6 mL) was added MsCl (255 mg, 2.2 mmol) under N₂ atmosphere at 0° C. and the mixture was stirred at room temperature for 3 hours. The mixture was quenched with saturated aq. NH₄Cl solution and extracted with DCM twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (523 mg, yield 79.7%) as a colorless oil. LC/MS (ESI) (m/z): 597 (M+H)⁺.

### Step 3: Benzyl (2S)-1-((phenoxathiine-3-carbonyl)glycyl)-4-(piperidin-1-ylmethyl) pyrrolidine-2-carboxylate (4)

To a mixture of benzyl (2S)-4-(((methylsulfonyl)oxy)methyl)-1-((phenoxathiine-3-carbonyl)glycyl)pyrrolidine-2-carboxylate (223 mg, 0.37 mmol) and piperidine (159 mg, 1.87 mmol) in DMSO (3 mL) was stirred at 100° C. for 3 hours. The mixture was diluted with EtOAc, washed with brine, dried over anhydrous Na₂SO₄, filtered, and concentrated under reduced pressure to dryness. The residue was purified by flash chromatography (silica gel, 0-80% EtOAc in PE) to give the title compound (100 mg, yield 45.7%) as a yellow oil. LC/MS (ESI) (m/z): 586 (M+H)⁺.

### Step 4: (2S)-1-((phenoxathiine-3-carbonyl)glycyl)-4-(piperidine-1-ylmethyl) pyrrolidine-2-carboxylic acid (5)

To a solution of benzyl (2S)-1-((phenoxathiine-3-carbonyl)glycyl)-4-(piperidin-1-ylmethyl)pyrrolidine-2-carboxylate (100 mg, 0.17 mmol) in MeOH (2 mL) and water (0.5 mL) was added LiOH·H₂O (14 mg, 0.34 mmol) and the mixture was stirred at room temperature for 2 hours. The mixture was acidified with IN aq. HCl to pH˜3 and extracted with CHCl₃/i-PrOH (v/v=3/1) twice. The combined organic layers were washed with brine, dried over anhydrous Na₂SO₄, filtered, and concentrated under reduced pressure to dryness to give the title compound (74 mg, yield 88.1%) as a yellow oil, which was used directly in the next step without further purification. LC/MS (ESI) m/z: 496 (M+H)⁺.

### Step 5: (2S)-1-((phenoxathiine-3-carbonyl)glycyl)-N-((R)-1-(1-(phenylsulfonyl)-1H-pyrrolo[3,2-c]pyridin-2-yl)ethyl)-4-(piperidin-1-ylmethyl)pyrrolidine-2-carboxamide (6)

To a mixture of (2S)-1-((phenoxathiine-3-carbonyl)glycyl)-4-(piperidin-1-ylmethyl) pyrrolidine-2-carboxylic acid (74 mg, 0.15 mol) and (R)-1-(1-(phenylsulfonyl)-1H-pyrrolo[3,2-c]pyridin-2-yl)ethan-1-amine hydrochloride (67 mg, 0.22 mmol) in DMF (1 mL) was added DIPEA (85 mg, 0.75 mmol) and HATU (97 mg, 0.22 mol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give the title compound (30 mg, yield 25.7%) as a brown oil, LC/MS (ESI) m/z: 779 (M+H)⁺.

### Step 6: (2S)-N-((R)-1-(1H-pyrrolo[3,2-c]pyridin-2yl)ethyl)-1-((phenoxathine-3-carbonyl)glycyl)-4-(piperidin-1-ylmethyl)pyrrolidine-2-carboxamide (Compound 185)

To a solution of (2S)-1-((phenoxathiine-3-carbonyl)glycyl)-N-((R)-1-(1-(phenylsulfonyl)-1H-pyrrolo[3,2-c]pyridin-2-yl)ethyl)-4-(piperidin-1-ylmethyl) pyrrolidine-2-carboxamide (30 mg, 0.039 mol) in MeOH (1 mL) and water (0.5 mL) was added LiOH·H₂O (3 mg, 0.077 mol) and the mixture was stirred at room temperature for 2 hours. The mixture was concentrated under reduced pressure to dryness. The residue was purified by prep-IIPLC to give Compound 185 (6.1 mg, yield 24.5%) as a white solid. ¹H NMR (400 MHz, CD₃OD): δ 8.62 (d, J=60.5 Hz, 1H), 8.14-7.81 (m, 1H), 7.50 (ddd, J=33.4, 14.6, 6.8 Hz, 2H), 7.31-6.99 (m, 6H), 6.67-6.49 (m, 1H), 5.36-5.23 (m, 11H), 4.54-3.89 (m, 4H), 3.49-3.33 (m, 11H), 2.70-2.43 (m, 7H), 2.29-1.85 (m, 11H), 1.81-1.38 (m, 10H). LC/MS (ESI) m/z: 639 (M+H)⁺. RT (Method A): 0.97 min.

Compound 184 was prepared based on Steps 3-6 in Scheme 53;

| Cmpd # | Reactant A | Reactant B | Reactant C | Characterization Data |
|---|---|---|---|---|
| 184 | 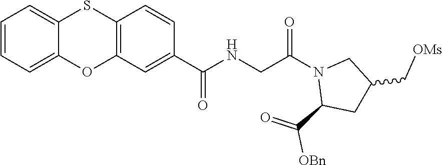 [SMILES: CS(=O)(=O)OCC1C[C@@H](C(=O)OCc2ccccc2)N(C(=O)CNC(=O)c2ccc3c(c2)Oc2ccccc2S3)C1] | 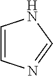 | 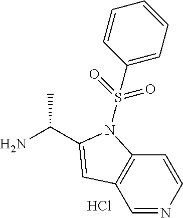 | ¹H NMR (400 MHz, CD₃OD): δ 8.68 (d, J = 19.6 Hz, 1H), 8.11-7.86 (m, 1H), 7.70 (t, J = 20.9 Hz, 1H), 7.53 (dd, J = 23.4, 16.2 Hz, 2H), 7.28-7.05 (m, 6H), 7.01 (s, 2H), 6.64-6.50 (m, 1H), 5.29 (dd, J = 20.2, 7.2 Hz, 1H), 4.59-4.38 (m, 1H), 4.27 (dd, J = 33.3, 12.4 Hz, 2H), 4.17-4.04 (m, 2H), 3.86 (dd, J = 15.9, 8.0 Hz, 1H), 3.48 (t, J = 8.6 Hz, 1H), 3.06-2.80 (m, 1H), 2.38-1.75 (m, 2H), 1.67-1.56 (m, 3H). LC/MS (ESI) m/z: 622 (M + H)⁺. RT (Method A): 0.97 min. |

### Scheme 54. Synthesis of (1S,3S,5S)-N-((R)-1-(1H-pyrrolo[3,2-c]pyridin-2-yl)ethyl)-2-((phenoxathiine-3-carbonyl)glycyl)-5-(thiazol-2-ylmethyl)-2-azabicyclo[3.1.0]hexane-3-carboxamide (Compound 186)

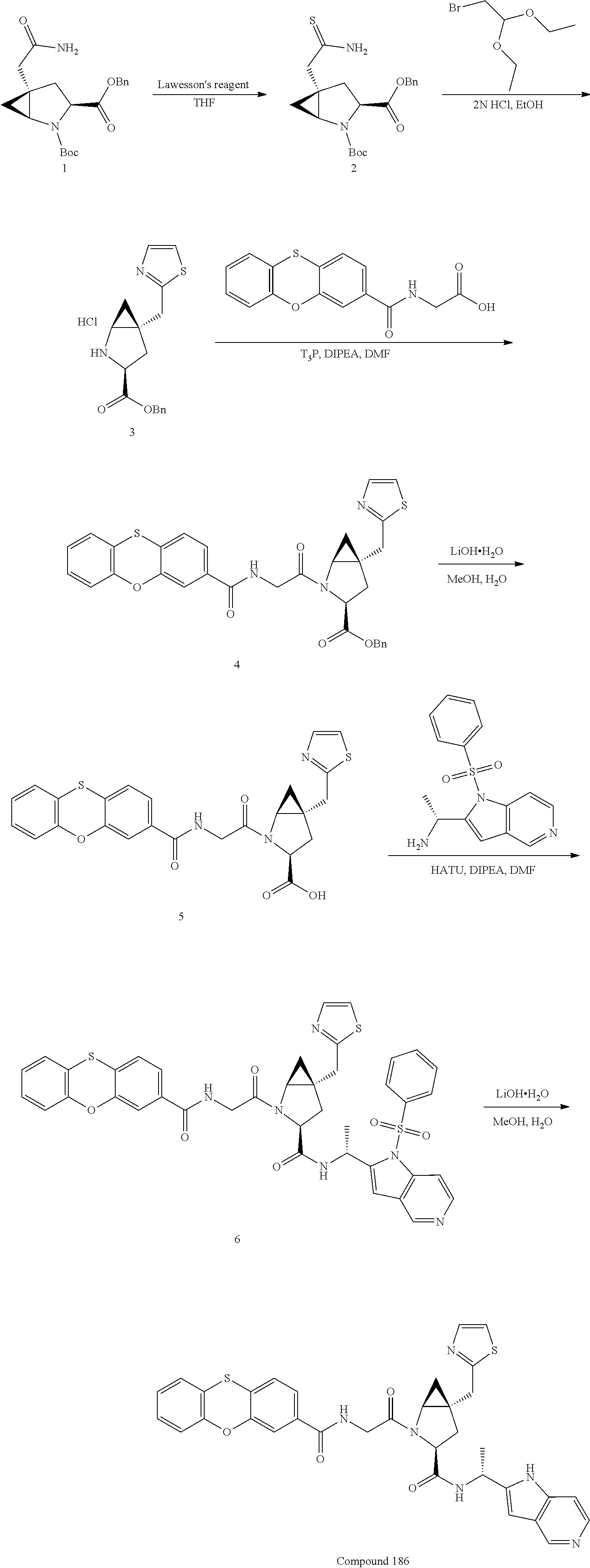

### Step 1: 3-Benzyl 2-(tert-butyl) (1S,3S,5S)-5-(2-amino-2-thioxoethyl)-2-azabicyclo[3.1.0]hexane-2,3-dicarboxylate (2)

To a solution of 3-benzyl 2-(tert-butyl) (1S,3 S,5S)-5-(2-amino-2-oxoethyl)-2-azabicyclo[3.1.0]hexane-2,3-dicarboxylate (300 mg, 0.80 nmol) in THF (5 mL) was added Lawesson s reagent (648 mg, 1.60 mmol) at 0° C. and the mixture was stirred at 0° C. 2 hours. The mixture was diluted with EtOAc, washed with saturated aq. NaHCO₃ solution ten times, dried over anhydrous Na₂SO₄, filtered, and concentrated to give the title compound (60 mg, yield 19.2%) as a white solid. LC/MS (ESI) m/z: 391 (M+H)⁺.

### Step 2: Benzyl (1S,3S,5S)-5-(thiazol-2-ylmethyl)-2-azabicyclo[3.1.0]hexane-3-carboxylate (3)

To a solution of 3-benzyl 2-(tert-butyl) (1S,3 S,5S)-5-(2-amino-2-thioxoethyl)-2-azabicyclo[3.1.0]hexane-2,3-dicarboxylate (55 mg, 0.14 mmol) and 2-bromo-1,1-diethoxyethane (56 mg, 0.28 mmol) in EtOH (5 mL) was added 2 N aq. HCl (0.5 mL) and the reaction mixture was stirred at 90° C. for 16 hours. The mixture was concentrated under reduced pressure to dryness to give the title compound (40 mg, yield 90.9%) as a brown solid, which was used directly in the next step without further purification. LC/MS (ESI) m/z: 315 (M+H)⁺.

### Step 3: Benzyl (1S,3S,5S)-2-((phenoxathiine-3-carbonyl)glycyl)-5-(thiazol-2-ylmethyl)-2-azabicyclo[3.1.0]hexane-3-carboxylate (4)

To a mixture of (phenoxathiine-3-carbonyl)glycine (21 mug, 0.07 mmol) and benzyl (1S,3S,5S)-5-(thiazol-2-ylmethyl)-2-azabicyclo[3.1.0]hexane-3-carboxylate hydrochloride (22 mg, 0.07 mmol) in DMF (3 mL) was added DIPEA (54 mug, 0.42 mmol) and T₃P (89 mg, 0.14 mmol, 50% wt. in EtOAc) at 0° C. under N₂ atmosphere and the mixture was stirred at room temperature for 1 hour. The mixture was quenched with saturated aq. NaHCO₃ solution and extracted with EtOAc twice. The combined organic layers were washed with brine, dried over anhydrous Na₂SO₄, filtered, and concentrated under reduced pressure to dryness. The residue was purified by prep-TLC (DCM:MeOH=30:1) to give the title compound (15 mg, yield 36.6%) as a yellow solid. LC/MS (ESI) m/z: 598 (M+H)⁺.

### Step 4: (1S,3S,5S)-2-((phenoxathiine-3-carbonyl)glycyl)-5-(thiazol-2-ylmethyl)-2-azabicyclo[3.1.0]hexane-3-carboxylic acid (5)

To a solution of benzyl (1S,3S,5S)-2-((phenoxathiine-3-carbonyl)glycyl)-5-(thiazol-2-ylmethyl)-2-azabicyclo[3.1.0]hexane-3-carboxylate (15 mg, 0.03 mmol) in MeOH (2.1 mL) and water (0.7 mL) was added LiOH·H₂O (1.9 mg, 0.05 mmol) and the reaction mixture was stirred at room temperature for 2 hours. The mixture was acidified with 1N aq. HCl to pH˜3 and extracted with EtOAc twice. The combined organic layers were dried over anhydrous Na₂SO₄, filtered, and concentrated under reduced pressure to dryness to give the title compound (10 mg, yield 76.9%) as a yellow solid, which was used directly in the next step without further purification. LC/MS (ESI) m/z: 508 (M+H)⁺.

### Step 5: (1S,3S,5S)-2-((phenoxathiine-3-carbonyl)glycyl)-N-((R)-1-(1-(phenylsulfonyl)-1H-pyrrolo[3,2-c]pyridin-2-yl)ethyl)-5-(thiazol-2-ylmethyl-2-azabicyclo[3.1.0]hexane-3-carboxamide (6)

To a mixture of (1S,3S,5S)-2-((phenoxathiine-3-carbonyl)glycyl)-5-(thiazol-2-ylmethyl)-2-azabicyclo[3.1.0]hexane-3-carboxylic acid (10 mg, 0.02 mmol) and (R)-1-(1-(phenylsulfonyl)-1H-pyrrolo[3,2-c]pyridin-2-yl)ethan-1-amine hydrochloride (11.9 mg, 0.04 mmol) in DMF (2 mL) was added DIPEA (15.5 mg, 0.12 mmol) and HATU (11.4 mg, 0.03 mmol) under N₂ atmosphere and the reaction mixture was stirred at room temperature for 2 hours. The mixture was diluted with EtOAc, washed with saturated aq·NH₄Cl solution and brine, dried over anhydrous Na₂SO₄, filtered, and concentrated under reduced pressure to dryness. The residue was purified by prep-TLC (DCM:MeOH=10:1) to give the title compound (12 mg, yield 75.0%) as a white solid. LC/MS (ESI) m/z: 791 (M+H).

### Step 6: (1S,3S,35S)-N-R((R)-1-(1H-pyrrolo[3,2-c]pyridin-2yl)ethyl)-2-((phenoxathiine-3-carbonyl)glycyl)-5-(thiazol-2-yl)methyl)-2-azabicyclo[3.1.0]hexane-3-carboxamide (Compound 186)

To a solution of (1S,3S,5S)-2-((phenoxathiine-3-carbonyl)glycyl)-N-((R)-1-(1-(phenylsulfonyl)-1H-pyrrolo[3,2-c]pyridin-2-yl)ethyl)-5-(thiazol-2-ylmethyl)-2-azabicyclo[3.1.0]hexane-3-carboxamide (12 mg, 0.02 mmol) in MeOH (2 mL) and water (1 mL) was added LiOH·H₂O (1.5 mg, 0.03 mmol), and the mixture was stirred at room temperature for 2 hours. The mixture was diluted with EtOAc, washed water and brine, dried over anhydrous NaSO₄, filtered, and concentrated under reduced pressure to dryness. The residue was purified by prep-HPLC to give Compound 186 (3 mg, yield 30.0%) as a white solid. ¹H NMR (400 MHz, CD₃OD): δ 8.65 (s, 1H), 7.92 (d, J=3.5 Hz, 1H), 7.74 (s, 1H), 7.57 (d, J=7.0 Hz, 2H), 7.53 (s, 1H), 7.26-7.19 (m, 2H), 7.15 (d, J=6.0 Hz, 2H), 7.09 (d, J=7.4 Hz, 1H), 7.03 (d, J=7.7 Hz, 1H), 6.51 (s, 1H), 5.25-5.20 (m, 1H), 4.57 (s, 2H), 4.28 (d. J=16.8 Hz, 1H), 3.72 (s, 1H), 3.02 (s, 2H), 2.55 (t, J=12.4 Hz, 1H), 2.14 (d, J=13.3 Hz, 1H), 1.57 (d, J=6.7 Hz, 3H), 1.52 (M, 1H), 1.14 (M, 1H). LC/MS (ESI) m/z: 651 (M+H)⁺. RT (Method A): 1.50 min.

### Scheme 55. Synthesis of (S)-N-((1H-pyrrolo[3,2-c]pyridin-2-yl)methyl)-1-((phenoxathiine-3-carbonyl)glycyl)pyrrolidine-2-carboxamide (Compound 188)

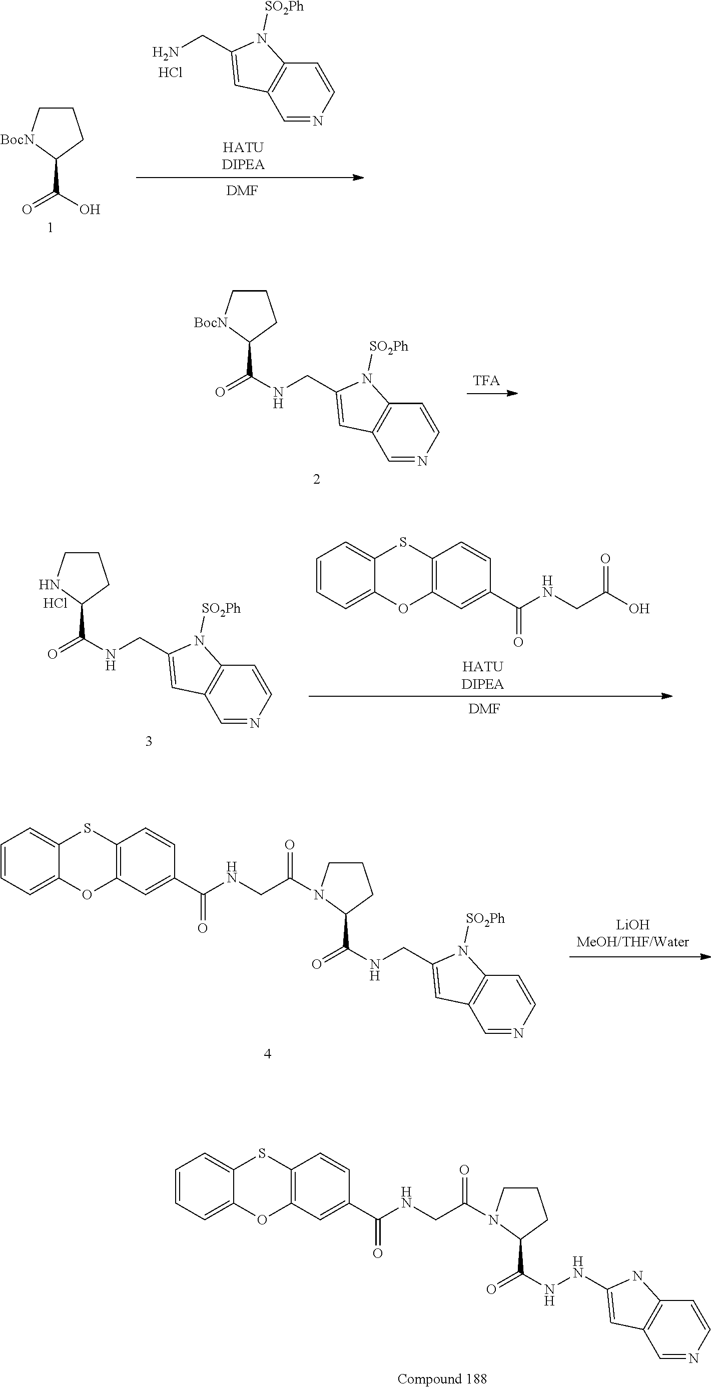

### Step 1: tert-Butyl (2S)-2-[[1-(benzenesulfonyl)pyrrolo[3,2-c]pyridin-2-yl]methylcarbamoyl]pyrrolidine-1-carboxylate

To a solution of (2S)-1-tert-butoxycarbonylpyrrolidine-2-carboxylic acid (45 mg, 0.21 mmol), [1-(benzenesulfonyl)pyrrolo[3,2-c]pyridin-2-yl]methanamine hydrochloride (50 mg, 0.17 mmol), and HATU (100 mg, 0.26 mmol) in DMF (1 mL) was added DIPEA (0.15 mL, 0.86 mmol) at room temperature. The reaction mixture was stirred for 2 hours and diluted with water (5 mL). The mixture was extracted with ethyl acetate three times. The combined organic layers were concentrated, and the residue was purified on silica gel (20% ethyl acetate in heptane) to give tert-butyl (2S)-2-[[1-(benzenesulfonyl)pyrrolo[3,2-c]pyridin-2-yl]methylcarbamoyl]pyrrolidine-1-carboxylate (70 mg, 83% yield) as a clear gel. LC/MS (ESI) m/z=485 (M+H)⁺.

### Step 2: (2S)-N-[[1-(benzenesulfonyl)pyrrolo[3,2-c]pyridin-2-yl]methyl]pyrrolidine-2-carboxamide

To a solution of tert-butyl (2S)-2-[[1-(benzenesulfonyl)pyrrolo[3,2-c]pyridin-2-yl]methylcarbamoyl]pyrrolidine-1-carboxylate (70 mg, 0.14 mmol) in DCM (0.5 ml) was added TFA (0.11 mL, 1.5 mmol) at room temperature. The reaction mixture was stirred for 30 minutes and concentrated under reduced pressure to give (2S)-N-[[1-(benzenesulfonyl)pyrrolo[3,2-c]pyridin-2-yl]methyl]pyrrolidine-2-carboxamide (55 mg, theory). This material was used in next step without further purification. LC/MS (ESI) m/z: 385 (M+H)⁺.

### Step 3: (2S)-N-[[1-(benzenesulfonyl)pyrrolo[3,2-c]pyridin-2-yl]methyl]-1-[2-(phenoxathiine-3-carbonylamino)acetyl]pyrrolidine-2-carboxamide

To a solution of 2-(phenoxathiine-3-carbonylamino)acetic acid (40 mg, 0.13 mmol), (2S)-N-[[1-(benzenesulfonyl)pyrrolo[3,2-c]pyridin-2-yl]methyl]pyridine-2-carboxamide (56 mg, 0.15 mmol), and HATU (76 mg, 0.2 mmol) in DMF (1 mL) was added DIPEA (0.14 mL, 0.80 mmol) at room temperature for 2 hours. The reaction mixture was diluted with water and stirred for 20 minutes at room temperature. The red solid was collected by filtration to give (2S)-N-[[1-(benzenesulfonyl)pyrrolo[3,2-c]pyridin-2-yl]methyl]-1-[2-(phenoxathiine-3-carbonylamino)acetyl]pyrrolidine-2-carboxamide (80 mg, 74% yield). LC/MS (ESI) m/z=668 (M+H)⁺.

### Step 4: (2S)-1-[2-(phenoxathiine-3-carbonylamino)acetyl]-N-(H-pyrrolo[3,2-c]pyridin-2-ylmethyl)pyrrolidine-2-carboxamide (Compound 188)

To a mixture of (2S)-N-[[1-(benzenesulfonyl)pyrrolo[3,2-c]pyridin-2-yl]methyl]-1-[2-(phenoxathiine-3-carbonylamino)acetyl]pyrrolidine-2-carboxamide (40 mg, 0.049 mmol) in THF (0.4 mL) and water (0.2 mL) was added LiOH·H₂O (4.2 mg, 0.10 nmol) at room temperature. The mixture was stirred at room temperature for 18 hours, LC/MS shown incomplete conversion. Three pipet drops of methanol was added and the reaction mixture was stirred for 1 hour. The reaction mixture was diluted with water and the yellow precipitates was collected by filtration. The solid was purified on silica gel (0 to 100% (contains 10% 7 N ammonia in methanol) in DCM) to give Compound 188 (10 mg, 0.01895 mmol, 38.59% yield) as a white solid. ¹H-NMR (400 MHz, DMSO-d6): δ 11.23 (s, 1H), 8.80-8.54 (m, 2H), 8.39 (t, J=5.8 Hz, 1H), 7.98 (d, J=5.7 Hz, 1H), 7.62-7.40 (m, 2H), 7.36-6.95 (m, 6H), 6.33 (s, 1H), 4.57-4.21 (m, 3H), 4.20-3.86 (m, 2H), 3.76-3.34 (m, 2H), 2.12-1.64 (m. 2H), 1.27-1.05 (m, 2H). LC/MS (ESI) m/z: 528.2 (M+H). RT (Method A): 1.28 min.

### Scheme 56. Synthesis of (5-((R)-1-((2S,4R)-4-(difluoromethoxy)-1-((4-phenoxybenzoyl)glycyl)pyrrolidine-2-carboxamido)ethyl)thiophen-3-yl)boronic acid (Compound 189)

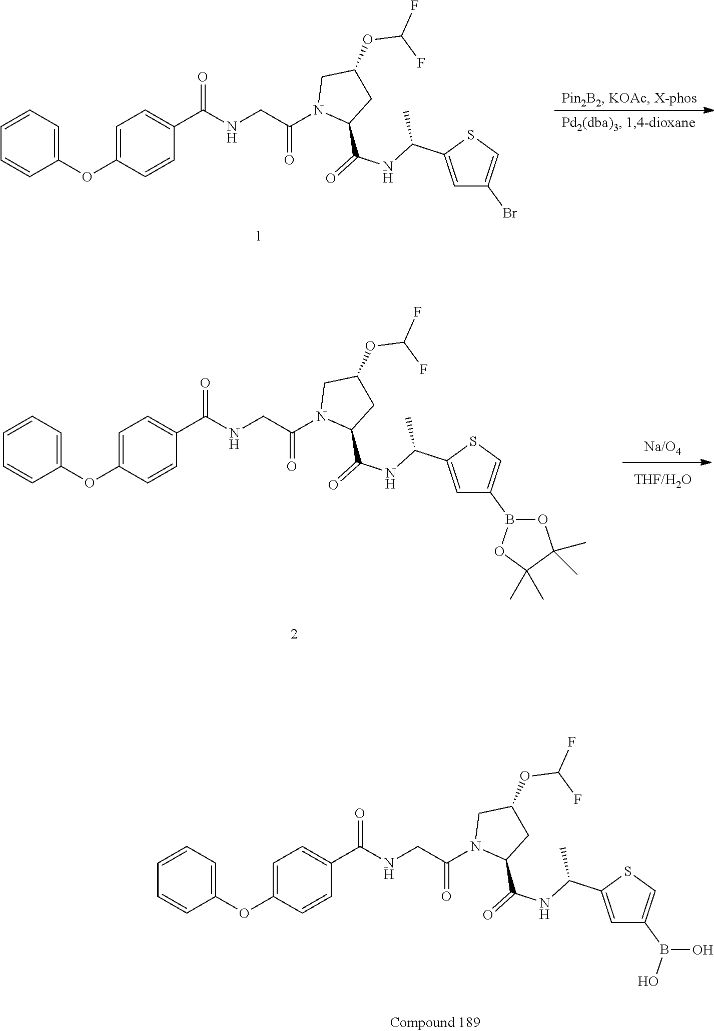 [SMILES: C[C@@H](NC(=O)[C@@H]1C[C@@H](OC(F)F)CN1C(=O)CNC(=O)c1ccc(Oc2ccccc2)cc1)c1cc(Br)cs1 ; C[C@@H](NC(=O)[C@@H]1C[C@@H](OC(F)F)CN1C(=O)CNC(=O)c1ccc(Oc2ccccc2)cc1)c1cc(B2OC(C)(C)C(C)(C)O2)cs1 ; C[C@@H](NC(=O)[C@@H]1C[C@@H](OC(F)F)CN1C(=O)CNC(=O)c1ccc(Oc2ccccc2)cc1)c1cc(B(O)O)cs1]

### Step 1: (2S,4R)-4-(difluoromethoxy)-1-((4-phenoxybenzoyl)glycyl))-N-((R)-1-(4-(4,4,5,5-tetramethyl-1,3,2-dioxaborolan-2-yl)thiophen-2-yl)ethyl)pyrrolidine-2-carboxamide (2)

To a mixture of (2S,4R)-N-((R)-1-(4-bromothiophen-2-yl)ethyl)-4-(difluoromethoxy)-1-((4-phenoxybenzoyl)glycyl)pyrrolidine-2-carboxamide (80 mug, 0.13 mmol) and Bis(pinacolato)diboron (132 mg, 0.52 mmol) in 1.4-dioxane (2 mL) was added KOAc (51 mg. 0.52 mmol), X-phos (10 mg, 0.02 mmol) and Pd₂(dba)₃ (36 mg, 0.04 mmol) under N₂ atmosphere, the mixture was degassed under N₂ atmosphere for three times and stirred under N₂ atmosphere at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (80 mg, yield 92.0%) as a white solid. LC/MS (ESI) m/z: 670 (M+H)⁺.

### Step 2: (5-((R)-1-((2S,4R)-4-(difluoromethoxy)-1-((4-phenoxybenzoyl)glycyl) pyrrolidine-2-carboxamido)ethyl)thiophen-3-yl)boronic acid (Compound 189)

To a solution of (2S,4R)-4-(difluoromethoxy)-1-((4-phenoxybenzoyl)glycyl)-N-((R)-1-(4-(4,4,5,5-tetramethyl-1,3,2-dioxaborolan-2-yl)thiophen-2-yl)ethyl)pyrrolidine-2-carboxamide (45 mg, 0.067 mol) in THF (1 mL) and water (1 mL) was added NaIO₄ (2 mg, 0.048 mol), and the mixture was stirred at room temperature for 6 hours. The mixture was concentrated under reduced pressure to dryness. The residue was purified by prep-HPLC to give Compound 189 (1.1 mg, yield 2.8%) as a white solid. U¹H NMR (400 MHz, CD₃OD): δ 7.88-7.83 (m, 2H), 7.69 (d, J=17.6 Hz, 1H), 7.41 (dd, J=8.5, 7.6 Hz, 2H), 7.24-7.17 (m, 2H), 7.08-7.04 (m, 2H), 7.02-6.98 (m, 2H), 6.48 (td, J=74.6, 15.4 Hz, 1H), 5.31 (dt, J=20.7, 6.9 Hz, 1H), 5.00-4.89 (m, 1H), 4.55 (t, J=7.6 Hz, 1H), 4.32-4.08 (m, 2H). 3.95 (dd, J=11.5, 4.7 Hz, 1H), 3.84 (d, J=12.0 Hz, 1H), 2.61-2.38 (m, 1H), 2.32-2.15 (m, 1H), 1.60 (dd, J=19.8, 6.9 Hz, 3H). LC/MS (ESI) m/z: 588 (M+H). RT (Method A): 1.91 min.

### Scheme 57. Synthesis of (S)-N-((R)-1-(1H-pyrrolo[3,2-c]pyridin-2-yl)ethyl)-5-((phenoxathiine-3-carbonyl)glycyl)-5-azaspiro[2.4]heptane-6-carboxamide (Compound 190)

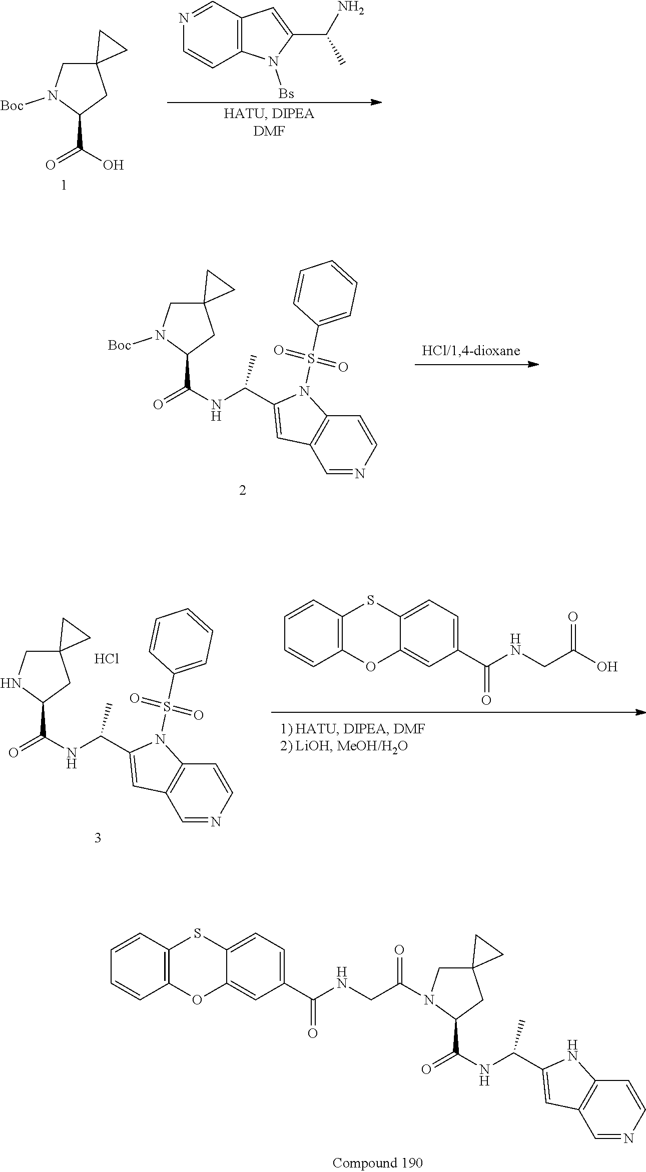

### Step 1: Tert-butyl (S)-6-(((R)-1-(1-(phenylsulfonyl)-1H-pyrrolo[3,2-c]pyridin-2-yl)ethyl)carbamoyl)-5-azaspiro[2.4]heptane-5-carboxylate (2)

To a mixture of (S)-5-(tert-butoxycarbonyl)-5-azaspiro[2.4]heptane-6-carboxylic acid (24 mg, 0.1 mmol) and (R)-1-(1-((4-bromophenyl)sulfonyl)-1H-pyrrolo[3,2-c]pyridin-2-yl)ethan-1-amine (57 mg, 0.15 mmol) in DMF (0.5 mL) was added DIPEA (43 mg, 0.33 mmol) and HATU (45 mg, 0.12 mmol) under N₂ atmosphere and the reaction mixture was stirred at room temperature for 1 hour. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=12:1) to give title compound (35 mg, yield 66.7%) as a white solid. LC/MS (ESI) m/z: 525 (M+H)⁺.

### Step 2: (S)-N-((R)-1-(1-(phenylsulfonyl)-H-pyrrolo[3,2-c]pyridin-2-yl)ethyl)-5-azaspiro[2.4]heptane-6-carboxamide hydrochloride (3)

A solution of tert-butyl (S)-6-(((R)-1-(1-(phenylsulfonyl)-1H-pyrrolo[3,2-c]pyridin-2-yl)ethyl)carbamoyl)-5-azaspiro[2.4]heptane-5-carboxylate (35 mg, 0.057 mmol) in DCM (2 mL, 4M) was added HCl/1,4-dioxane (0.5 mL, 2.0 mmol) and the mixture was stirred under N₂ atmosphere at room temperature for 2 hours. The reaction mixture was concentrated to dryness under reduced pressure to give the title compound (20 mg, yield 76.1%) as a white solid. LC/MS (ESI) (m/z): 425 (M+H)⁺.

### Step 3: (S)-N-((R)-1-(1H-pyrrolo[3,2-c]pyridin-2-yl)ethyl)-5-((phenoxathiine-3-carbonyl)glycyl)-5-azaspiro[2.4]heptane-6-carboxamide (Compound 190)

To a mixture of (phenoxathiine-3-carbonyl)glycine (12 mg, 0.040 mmol) and (S)-N-((R)-1-(1-(phenylsulfonyl)-1H-pyrrolo[3,2-c]pyridin-2-yl)ethyl)-5-azaspiro[2.4]heptane-6-carboxamide hydrochloride (20 mg, 0.043 mmol) in DMF (0.5 mL) was added DIPEA (26 mg, 0.2 mmol) and HATU (23 mg, 0.06 mmol) under N₂ atmosphere and the reaction mixture was stirred at room temperature for 1 hour. To the above mixture were added MeOH (3 mL) and water (1 mL) followed by LiOH·H₂O (2 mg, 0.05 mmol) and the resulting mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=7:1) and further purified by prep-HPLC to give Compound 190 (3.5 mg, yield 15.4%) as a white solid. ¹H NMR (400 MHz, CD₃OD) δ 8.97 (s, 1H), 8.11 (d, J=6.6 Hz, 1H), 7.67 (d, J=6.6 Hz, 1H), 7.58 (d, J=8.1 Hz, 1H), 7.51 (s, 1H), 7.25 (d, J=8.1 Hz, 1H), 7.18 (dd, J=12.6, 7.6 Hz, 2H), 7.11-7.07 (m, 1H), 7.03-6.95 (m, 2H), 5.35 (q, J=6.7 Hz, 1H), 4.62 (t, J=7.0 Hz, 1H), 4.25 (d, J=16.6 Hz, 1H), 4.15 (d, J=16.7 Hz, 1H), 3.72 (d, J=9.8 Hz, 1H), 3.62 (d, J=9.9 Hz, 1H), 2.21 (dd, J=12.4, 8.4 Hz, 1H), 2.05 (dd, J=12.3, 5.9 Hz, 1H), 1.65 (d, J=7.0 Hz, 3H), 0.74 (s, 2H), 0.67 (s, 2H). LC/MS (ESI) m/z: 568 (M+H)⁺.

### Scheme 58. Synthesis of (2S,4R)-4-(difluoromethoxy)-N-(indolin-5-ylmethyl)-1-((phenoxathiine-3-carbonyl)glycyl)pyrrolidine-2-carboxamide (Compound 197)

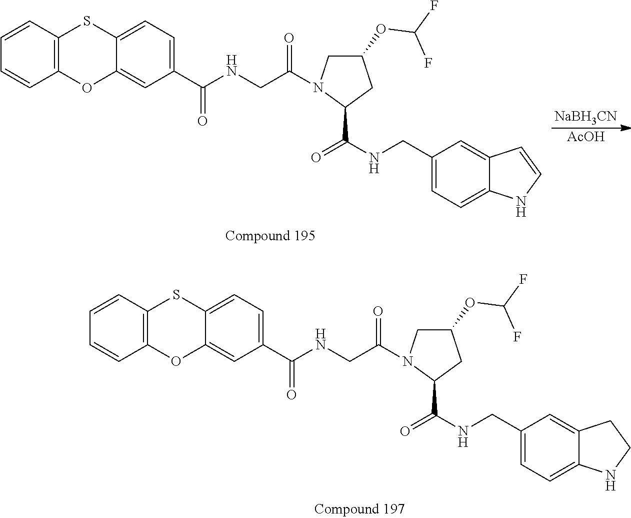 [SMILES: "AcOH" ; O=C(NCC(=O)N1C[C@H](OC(F)F)C[C@H]1C(=O)NCc1ccc2[nH]ccc2c1)c1ccc2c(c1)Oc1ccccc1S2 ; "NaBH3CN" ; O=C(NCC(=O)N1C[C@H](OC(F)F)C[C@H]1C(=O)NCc1ccc2c(c1)CCN2)c1ccc2c(c1)Oc1ccccc1S2]

To a solution of (2S,4R)-N-((1H-indol-5-yl)methyl)-4-(difluoromethoxy)-1-((phenoxathiine-3-carbonyl)glycyl)pyrrolidine-2-carboxamide (Compound 195; 6 mg, 0.01 mol) in AcOH (0.5 mL) was added NaBH₃CN (2 mg, 0.032 mol) at 0° C. and the mixture was stirred at 30° C. for 1 hour. The mixture was filtered, and the filtrate was concentrated to dryness. The residue was purified by prep-HPLC to give Compound 197 (1.5 mg, yield 24.9%) as a white solid. ¹H NMR (400 M1 Hz, CD₃OD): δ 7.52 (dd, J=8.1, 1.7 Hz, 1H), 7.48 (d, J=1.5 Hz, 1H), 7.22-7.13 (m, 3H), 7.05 (dd, J=16.4, 7.8 Hz, 3H), 6.96-6.90 (m, 1H), 6.69-6.58 (m, 1H), 6.57-6.27 (m, 1H), 4.98 (d, J=3.5 Hz, 1H), 4.54 (d. J=7.7 Hz, 1H), 4.26 (d, J=2.9 Hz, 2H), 4.15 (dd, J=28.0, 23.4 Hz, 2H). 3.94 (dd, J=11.4, 4.6 Hz, 1H), 3.85-3.78 (m, 1H), 3.40 (t, J=8.3 Hz, 2H), 2.91 (dd, J=15.3, 7.0 Hz, 2H), 2.49-2.36 (m, 1H), 2.24-2.16 (m, 1H). LC/MS (ESI) m/z: 595 (M+H)⁺. RT (Method A): 1.60 min.

Compound 198 was prepared based on Scheme 58:

| Cmpd # | Reactant A | Characterization Data |
|---|---|---|
| 198 | 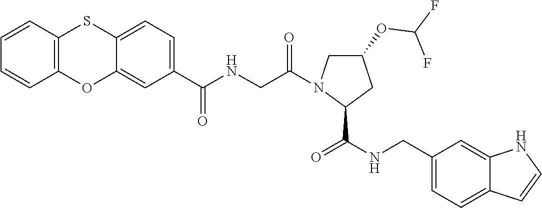 [SMILES: O=C(NCC(=O)N1C[C@H](OC(F)F)C[C@H]1C(=O)NCc1ccc2cc[nH]c2c1)c1ccc2c(c1)Oc1ccccc1S2] | ¹H NMR (400 MHz, CD₃OD): δ 7.50 (dd, J = 18.5, 3.6 Hz, 2H), 7.26-7.11 (m, 3H), 7.09-6.96 (m, 3H), 6,69-6.27 (m, 3H), 4.95 (d, J = 32.8 Hz, 1H), 4.69-4.55 (m, 1H), 4.39-4.26 (m, 2H), 4.17 (dd, J = 28.8, 12.2 Hz, 2H), 3.94 (dt, J = 15.0, 7.7 Hz, 1H), 3.87-3.79 (m, 1H), 3.41 (dd, J = 18.5, 8.6 Hz, 2H), 2.91 (q, J = 8.5 Hz, 2H), 2.65-2.41 (m, 1H), 2.37-2.19 (m, 1H). LC/MS (ESI) m/z: 595 (M + H)⁺. RT (Method A): 1.66 min. |

### Scheme 59. Synthesis of 6-(((3S)-5-(((R)-1-(1H-pyrrolo[3,2-c]pyridin-2-yl)ethyl)carbamoyl)-1-((phenoxathiine-3-carbonyl)glycyl)pyrrolidin-3-yl)methoxy)hexanoic acid (Compound 204)

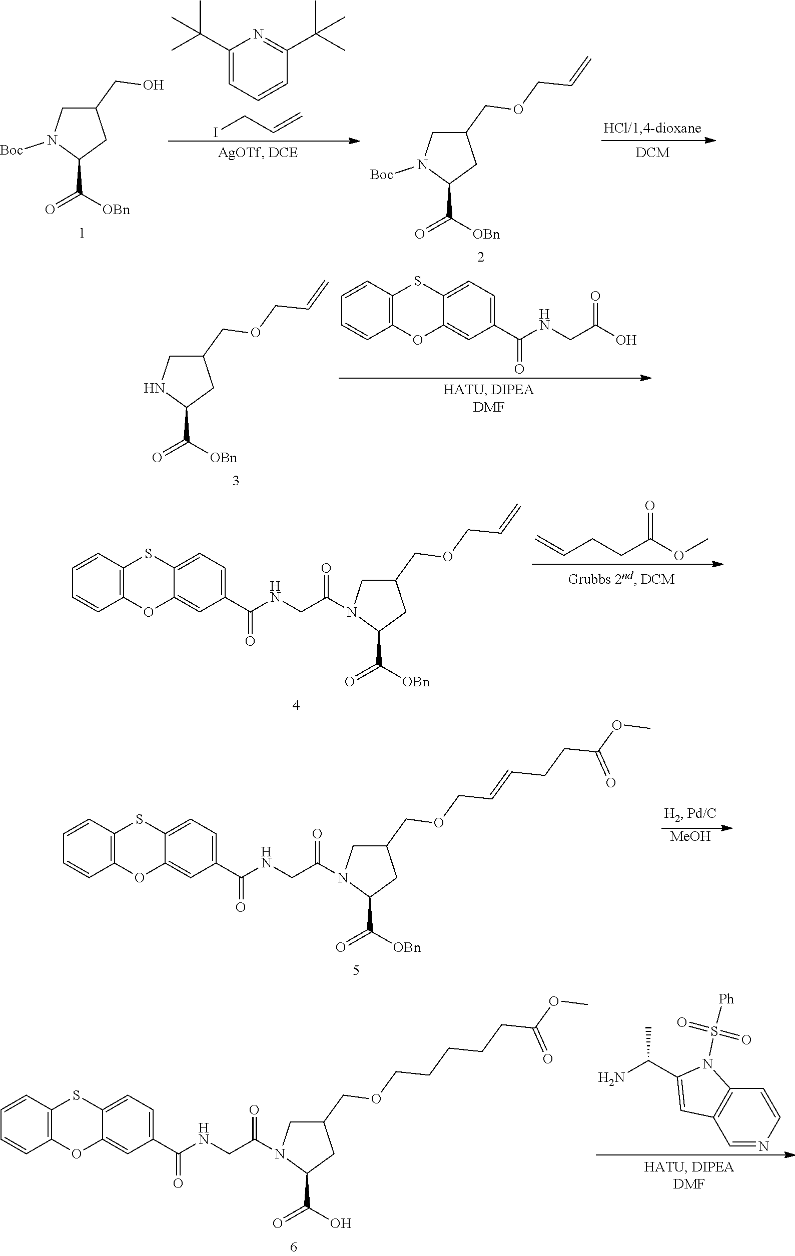

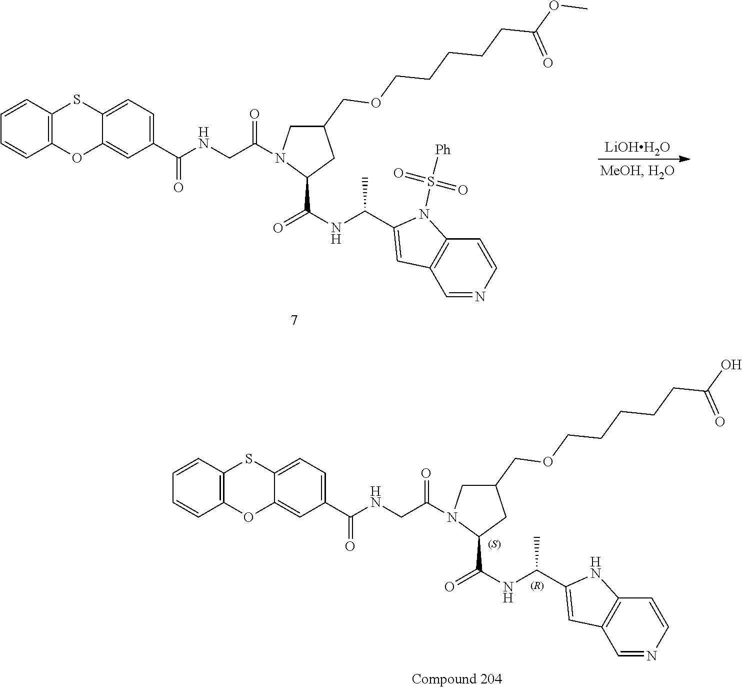

### Step 1: 2-Benzyl 1-(tert-butyl)(2S)-4-((allyloxy)methyl)pyrrolidine-1,2-dicarboxylate (2)

To a solution of 2-benzyl 1-(tert-butyl) (2S)-4-(hydroxymethyl)pyrrolidine-1,2-dicarboxylate (2 g, 5.97 mmol) in DCE (20 mL) was added AgOTf (2.3 g, 8.96 mmol) followed by 2,6-di-tert-butylpyridine (3.4 g, 17.9 mmol) and 3-iodoprop-1-ene (3 g, 17.9 mmol) and the mixture was stirred under N₂ atmosphere at 30° C. for 16 hours. The mixture was filtered, and the filtrate was concentrated to dryness. The residue was purified by flash chromatogmphy (silica gel, 0-15% EtOAc in PE) to give title compound (1.46 g, yield 65.2%) as a yellow oil. LC/MS (ESI) (m/z): 376 (M+H)⁺.

### Step 2: Benzyl (2S)-4-((allyloxy)methyl)pyrrolidine-2-carboxylate hydrochloride (3)

A solution of 2-benzyl 1-(tert-butyl) (2S)-4-((allyloxy)methyl)pyrrolidine-1,2-dicarboxylate (1 g, 2.67 mmol) in HCl1,4-dioxane (15 mL, 4M) was stirred under N₂ atmosphere at room temperature for 2 hours. The reaction mixture was concentrated to dryness under reduced pressure to give the title compound (830 mg, yield 100%) as a yellow solid, which was used directly in the next step without further purification. LC/MS (ESI) m/z: 276 (M+H)⁺.

### Step 3: Benzyl (2S)-4-((allyloxy)methyl)-1-((phenoxathiine-3-carbonyl)glycyl) pyrrolidine-2-carboxylate (4)

To a mixture of (phenoxathiine-3-carbonyl)glycine (803 mg, 2.67 mmol) and benzyl (2S)-4-((allyloxy)methyl)pyrrolidine-2-carboxylate hydrochloride (733 mg, 2.67 mmol) in DMF (10 mL) was added DIPEA (2.07 g, 16 mmol) and HATU (1.32 g, 3.47 mmol) at 0° C. and the mixture was stirred at room temperature for 1 hour. The mixture was diluted with EtOAc, washed with brine, dried over anhydrous Na₂SO₄, filtered, and concentrated under reduced pressure to dryness. The residue was purified by flash chromatography (silica gel, 0-50% EtOAc in PE) to give the title compound (890 mg, yield 59.9%) as a yellow oil. LC/MS (ESI) m/z: 559 (M+H)⁺.

### Step 4: Benzyl (2S)-4-((((E)-6-methoxy-6-oxohex-2-en-1-yl)oxy)methyl)-1-((phenoxathiine-3-carbonyl)glycyl)pyrrolidine-2-carboxylate (5)

To a mixture of benzyl (2S)-4-((allyloxy)methyl)-1-((phenoxathiine-3-carbonyl 1) glycyl)pyrrolidine-2-carboxylate (325 mg, 0.58 mmol) and methyl pent-4-enoate (100 mg, 0.88 mmol) in DCM (100 mL) was added Grubbs 2^{nd} catalyst (192 mg, 0.23 mmol) at room temperature under N₂ atmosphere and the mixture was stirred at room temperature for 16 hours. The mixture was concentrated under reduced pressure to dryness. The residue was purified by flash chromatography (silica gel, 0-50% EtOAc in PE) to give the title compound (120 mg, yield 31.9%) as a colorless oil. LC/MS (ESI) m/z: 645 (M+H)⁺.

### Step 5: (2S)-4-(((6-methoxy-6-oxohexyl)oxy)methyl)-1-((phenoxathiine-3-carbonyl)glycyl)pyrrolidine-2-carboxylic acid (6)

To a solution of benzyl (2S)-4-((((E)-6-methoxy-6-oxohex-2-en-1-yl)oxy)methyl)-1-((phenoxathiine-3-carbonyl)glycyl)pyrrolidine-2-carboxylate (100 mg, 0.16 mmol) in MeOH (2 mL) was added Pd/C (10 mg, 10% wt.), the mixture was degassed under N₂ atmosphere for three times and stirred under a H₂ balloon at 25° C. 1 hour. The mixture was filtered, and the filtrate was concentrated under reduced pressure to dryness to give the title compound (55 mg, yield 63.9%) as a colorless oil, which was used directly in the next step without further purification. LC/MS (ESI) m/z: 557 (M+H)⁺.

### Step 6: Methyl 6-(((3S)-1-((phenoxathiine-3-carbonyl)glycyl)-5-(((R)-1-(1-(phenylsulfonyl)-1H-pyrrolo[3,2-c]pyridin-2-yl)ethyl)carbamoyl)pyrrolidin-3-yl)methoxy)hexanoate (7)

To a mixture of (2S,4S)-4-(((6-methoxy-6-oxohexyl)oxy)methyl)-1-((phenoxathiine-3-carbonyl)glycyl)pyrrolidine-2-carboxylic acid (52 mg, 0.08 mmol) and (R)-1-(1-(phenylsulfonyl)-1H-pyrrolo[3,2-c]pyridin-2-yl)ethan-1-amine (37 mg, 0.12 mmol) in DMF (2 mL) was added DIPEA (40 mg, 0.11 mmol) and HATU (62 mg, 0.48 mmol) at 0° C. under N₂ atmosphere and the mixture was stirred at room temperature for 1 hour. The mixture was diluted with EtOAc, washed with brine, dried over anhydrous Na₂SO₄, filtered, and concentrated under reduced pressure to dryness. The residue was purified by prep-TLC (DCM:MeOH=15:1) to give the title compound (35 mg, yield 44.9%) as a yellow oil. LC/MS (ESI) m/z: 840 (M+H)⁺.

### Step 8: 6-(((3S)-5-(((R)-1-(1H-pyrrolo[3,2-c]pyridin-2-yl)ethyl)carbamoyl)-1-((phenoxathiine-3-carbonyl)glycyl)pyrrolidin-3-yl)methoxy)hexanoic acid (Compound 204)

To a solution of methyl 6-(((3S)-1-((phenoxathiine-3-carbonyl)glycyl)-5-(((R)-1-(1-(phenylsulfonyl)-1H-pyrrolo[3,2-c]pyridin-2-yl)ethyl)carbamoyl)pyrrolidin-3-yl) methoxy)hexanoate (32 mg, 0.04 mmol) in MeOH (2 mL) and water (0.7 mL) was added LiOH·H₂O (3 mg, 0.06 mmol) and the mixture was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by prep-HPLC to give Compound 204 (1.2 mg, yield 4.6%) as a white solid. ¹H NMR (400 MHz, CD₃OD): δ 8.66 (s, 3H), 7.90 (d, J=5.9 Hz, 2H), 7.61 (s, 1H), 7.58 (d, J=2.4 Hz, 3H), 7.26 (d, J=8.0 Hz, 2H), 7.20 (s, 3H), 7.17 (d, J=7.8 Hz, 3H), 7.14 (s, 3H), 7.11 (d, J=7.9 Hz, 2H), 7.07 (d, J=6.1 Hz, 2H), 7.03 (d, J=8.3 Hz, 3H), 6.53 (s, 3H), 5.31 (d, J=6.9 Hz, 3H), 4.40 (dd, J=20.4, 12.5 Hz, 5H), 4.13 (d, J=16.7 Hz, 3H), 4.00-3.88 (m, 3H), 3.52-3.49 (m, 3H), 3.47 (d, J=4.3 Hz, 5H), 3.43 (d, J=7.5 Hz, 6H), 2.64 (s, 3H), 2.46-2.32 (m, 3H), 1.81 (dd, J=22.2, 10.0 Hz, 3H), 1.70 (d, J=6.8 Hz, 2H), 1.68-1.64 (m, 5H), 1.61 (d, J=7.1 Hz, 14H), 1.57 (d, J=8.1 Hz, 5H). LC/MS (ESI) m/z: 686 (M+H)⁺. RT (Method A): 1.56 min.

### Scheme 60. Synthesis of (S)-N-((1H-pyrrolo[3,2-c]pyridin-2-yl)methyl)-7-((dibenzo[b,d]furan-3-carbonyl)glycyl)-1,4-dioxa-7-azaspiro[4.4]nonane-8-carboxamide (Compound 213)

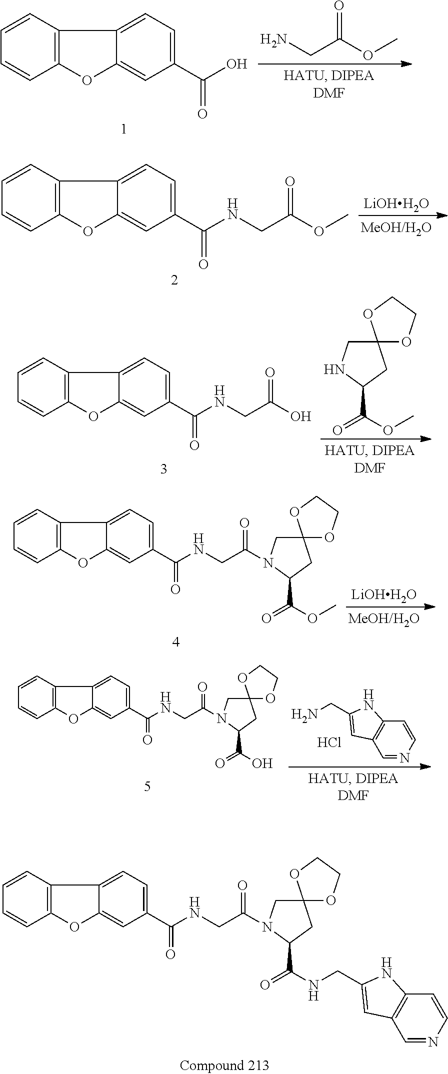

### Step 1: Methyl (dibenzo[b,d]furan-3-carbonyl)glycinate (2)

To a mixture of dibenzo[b,d]furan-3-carboxylic acid (110 mg, 0.52 mmol) and methyl glycinate (11 mg, 0.075 mmol) in DMF (0.7 mL) was added DIPEA (44 mg, 0.34 mmol) and HATU (31 mg, 0.082 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (10% EtOAc in PE) to give the title compound (100 mg, yield 68.0%) as a white solid. LC/MS (ESI) m/z: 284 (M+H)⁺.

### Step 2: (Dibenzo[b,d]furan-3-carbonyl)glycine (3)

To a solution of methyl (dibenzo[b,d]furan-3-carbonyl)glycinate (100 mg, 0.35 mmol) in MeOH/H₂O (2 mL, v/v=1/1) was added LiOH·H₂O ((48 mg, 1.2 mmol) and the mixture was stirred at room temperature for 2 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (92 mg, yield 97.1%) as a white solid, which was used directly in the next step without further purification. LC/MS (ESI) m/z: 270 (M+H)⁺.

### Step 3: Methyl (S)-7-((dibenzo[b,d]furan-3-carbonyl)glycyl)-1,4-dioxa-7-azaspiro[4.4]nonane-8-carboxylate (4)

To a mixture of (dibenzo[b,d]furan-3-carbonyl)glycine (50 mg, 0.19 mmol) and methyl (S)-1,4-dioxa-7-azaspiro[4.4]nonane-8-carboxylate (38 mg, 0.20 mmol) in DMF (0.7 mL) was added DIPEA (120 mg, 0.93 mmol) and HATU (85 mg, 0.22 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (4% MeOH in DCM) to give the title compound (50 mg, yield 61.4%) as a white solid. LC/MS (ESI) m/z: 439 (M+H)⁺.

### Step 4: (S)-7-((dibenzo[b,d]furan-3-carbonyl)glycyl)-1,4-dioxa-7-azaspiro[4.4]nonane-8-carboxylic acid (5)

To a solution of methyl (S)-7-((dibenzo[b,d]furan-3-carbonyl)glycyl)-1,4-dioxa-7-azaspiro[4.4]nonane-8-carboxylate (50 mg, 0.11 mmol) in MeOH/H₂O (2 mL, v/v=1/1) was added LiOH·H₂O (24 mg, 0.60 mmol) and the mixture was stirred at room temperature for 2 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (47 mg, yield 96.5%) as a white solid, which was used directly in the next step without further purification. LC/MS (ESI) m/z: 425 (M+H)⁺.

### Step 5: (S)-N-((1H-pyrrolo[3,2-c]pyridin-2-yl)methyl)-7-((dibenzo[b,d]furan-3-carbonyl)glycyl)-1,4-dioxa-7-azaspiro[4.4]nonane-8-carboxamide (Compound 213)

To a mixture of (S)-7-((dibenzo[b,d]furan-3-carbonyl)glycyl)-1,4-dioxa-7-azaspiro[4.4]nonane-8-carboxylic acid (40 mg, 0.094 mmol) and (1H-pyrrolo[3,2-c]pyridin-2-yl)methanamine (14 mg, 0.094 mmol) in DMF (0.7 mL) was added DIPEA (61 mg, 0.47 mmol) and HATU (42 mg, 0.11 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by prep-TLC (8% MeOH in DCM) and further purified by prep-HPLC to give Compound 213 (7 mg, yield 13.4%) as a white solid. ¹H NMR (400 MHz, CD₃OD): δ 8.63 (d, J=43.6 Hz, 1H), 8.09-8.03 (m, 3H), 7.87-7.82 (m, 2H), 7.63 (d, J=8.3 Hz, 1H), 7.56 (t, J=7.8 Hz, 1H), 7.40 (d, J=7.5 Hz, 1H), 7.17 (d, J=5.8 Hz, 1H), 6.55 (d, J=30.9 Hz, 1H), 4.62 (dt, J=24.1, 16.0 Hz, 4H), 4.23 (d, J=4.0 Hz, 2H), 4.01 (d, J=3.4 Hz, 3H), 3.83 (t, J=7.0 Hz, 2H), 2.46 (dd, J=13.1, 8.9 Hz, 1H), 2.28 (dd, J=13.2, 6.2 Hz, 1H). LC/MS (ESI) m/z: 554 (M+H)⁺. RT (Method A): 1.21 min.

### Scheme 61. Synthesis of (2S,4R)-N-((1H-pyrrolo[3,2-c]pyridin-2-yl)methyl)-4-(difluoromethoxy)-1-((3-(4-isopropylphenoxy)benzoyl)glycyl)pyrrolidine-2-carboxamide (Compound 215)

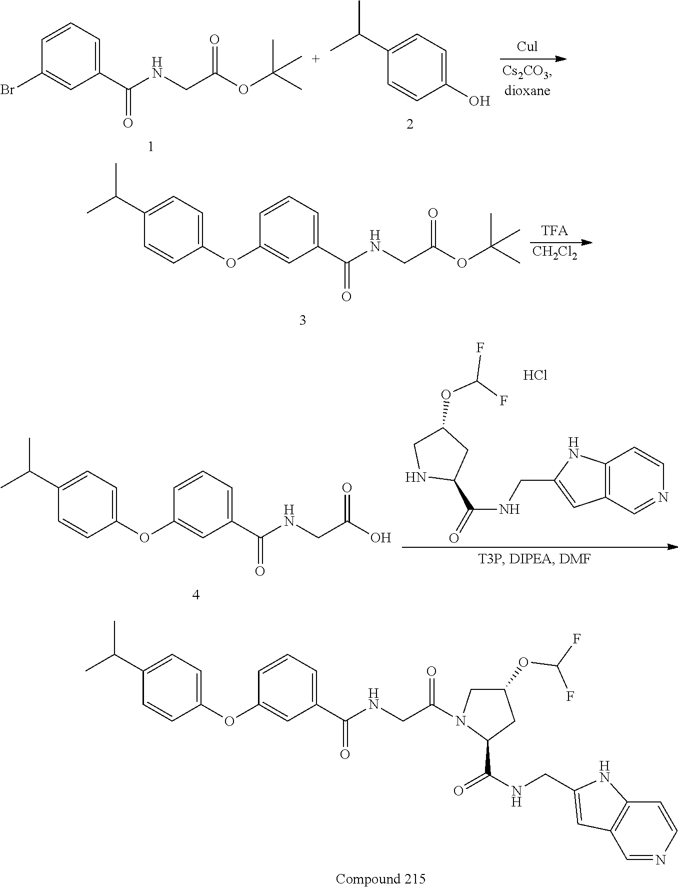

### Step 1: tert-butyl (3-(4-isopropylphenoxy)benzoyl)glycinate (3)

A mixture of tert-butyl 2-[(3-bromobenzoyl)amino]acetate (50.00 mg, 0.1591 mmol, 1.0 equiv.), isopropylphenol (43.35 mg, 0.3183 mmol, 2 equiv.), copper(I) iodide (3.031 mg, 0.01592 mmol, 0.1 equiv.), N,N-dimethylglycine hydrochloride (4.442 mg, 0.03182 mmol, 0.2 equiv.), cesium carbonate (0.2074 g, 0.6365 mmol, 4 equiv.) in 1,4-dioxane (3 mL) was stirred in a microwave reactor at 110° C. for 12 h. The reaction mixture was cooled to room temperature, diluted with water, extracted with EtOAc, washed with brine, dried over Na₂SO₄, and concentrated. Purification by combi-Flash; 12 g column, solvent A=hexanes, solvent B=EtOAc. 100% A to 15% B gave tert-butyl 2-[[3-(4-isopropylphenoxy)benzoyl]amino]acetate (46.90 mg, 0.1270 mmol, 100 mass %, 79.78% yield). LC/MS (ESI) m/z: 370 (M+H)⁺.

### Step 2: (3-(4-isopropylphenoxy)benzoyl)glycine (4)

TFA (0.5 mL) was added to a solution of tert-butyl 2-[[3-(4-isopropylphenoxy)benzoyl]-amino]acetate (30.00 mg, 0.08121 mmol, 1.0 equiv.) in CH₂Cl₂ (1 mL) at ice-bath temperature. The reaction mixture was stirred at room temperature for 4 hours and then concentrated to dryness to give 2-[[3-(4-isopropylphenoxy)benzoyl]amino]acetic acid (25.45 mg, 0.08121 mmol, 99.99% yield) as a clear oil which was used in the next step without further purification. LC/MS (ESI) m/z: 314 (M+H)⁺.

### Step 3. (2S,4R)-N-((1H-pyrrolo[3,2-c]pyridin-2-yl)methyl)-4-(difluoromethoxy)-1-((3-(4-isopropylphenoxy)benzoyl)glycyl)pyrrolidine-2-carboxamide (Compound 215)

To a solution of 2-[[3-(4-isopropylphenoxy)benzoyl]amino]acetic acid (9.037 mg, 0.02884 mmol, 1.0 equiv.) in DMF (2 mL) was added (2S,4R)-4-(difluoromethoxy)-N-(1H-pyrrolo[3,2-c]pyridin-2-ylmethyl)pyrrolidine-2-carboxamide;hydrochloride (10.00 mg, 0.02884 mmol, 1.0 equiv.), T3P (50 mass %) in ethyl acetate (25.90 μL, 0.0433 mmol, 1.5 equiv.), and DIPEA (15.10 μL, 0.0866 mmol, 3.0 equiv.). The reaction mixture was stirred at room temperature for 30 minutes and then purified directly by prep HPLC to give Compound 215 (2.837 mg, 0.004685 mmol, 16.25% yield). LC/MS (ESI) m/z: 606 (M+H)⁺. ¹H NMR (400 MHz, DMSO-d6): δ 11.23 (s, 1H), 9.10-8.43 (m, 2H), 8.20 (s, 1H), 8.03 (d, J=5.6 Hz, 1H), 7.64 (t, J=8.9 Hz, 1H), 7.53-7.40 (m, 1H), 7.27 (t, J=7.9 Hz, 2H), 7.18 (dd, J=8.1, 2.8 Hz, 2H), 6.97 (dd, J=8.8, 7.0 Hz, 1H), 6.81 (s, 1H), 6.38 (s, 1H), 4.97 (s, 1H), 4.56-4.30 (m, 3H), 4.18 (dd, J=16.7, 5.7 Hz, 1H), 4.01 (dd, J=17.0, 5.5 Hz, 1H), 3.88 (d, J=11.2 Hz, 1H), 3.85-3.65 (m, 11H), 2.89 (p, J=7.0 Hz, 1H), 2.32 (td, J=9.5, 6.3 Hz, 1H), 2.14 (dd, J=13.6, 6.7 Hz, 1H), 1.56-1.34 (m, 2H), 1.20 (d, J=7.0 Hz, 6H). RT (Method A): 1.85 min.

The following compounds were prepared based on Scheme 61:

| Cmpd # | Reactant A | Reactant B | Reactant C | Characterization Data |
|---|---|---|---|---|
| 244 | 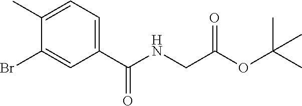 | 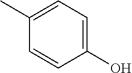 | 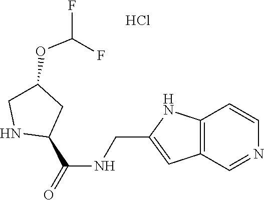 [SMILES: O=C(NCc1cc2cnccc2[nH]1)[C@@H]1C[C@@H](OC(F)F)CN1 ; Cl] | ¹H NMR (400 MHz, DMSO-d6): δ 11.21 (s, 1H), 8.65 (d, J - 14.2 Hz, 2H), 8.24 (d, J = 2.5 Hz, 1H), 8.02 (d, J = 5.4 Hz, 1H), 7.62 (t, J = 9.7 Hz, 1H), 7.42 (d, J = 7.7 Hz, 2H), 7.35 (d, J = 9.5 Hz, 1H), 6.83 (q, J = 9.7 Hz, 3H), 6.37 (s, 1H), 4.96 (s, 1H), 4.65 (d, J = 7.8 Hz, 1H), 4.52-4.25 (m, 3H), 4.15 (dd, J = 16.8, 5.6 Hz, 1H), 3.97 (d, J = 16.9 Hz, 1H), 3.82-3.66 (m, IH), 2.26 (s, 3H), 2.15 (s, 3H). LC/MS (ESI) m/z: 592 (M + H)⁺. RT (Method A): 1.65 min. |
| 260 | 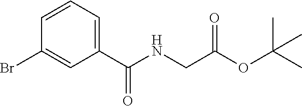 | 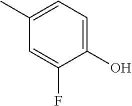 | 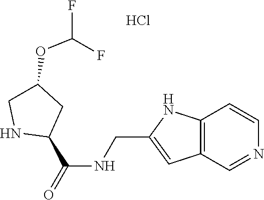 [SMILES: O=C(NCc1cc2cnccc2[nH]1)[C@@H]1C[C@@H](OC(F)F)CN1 ; Cl] | ¹H NMR (400 MHz, DMSO-d6): δ 11.40 (s, 1H), 9.16-8.47 (m, 2H), 8.30- 7.89 (m, 1H), 7.62 (t, J = 8.7 Hz, 1H), 7.48 (t, J = 7.9 Hz, 1H), 7.38 (d, J = 13.8 Hz, 1H), 7.30-7.20 (m, 1H), 7.20-7.10 (m, 2H), 7.05 (d, J = 8.2 Hz, 1H), 6.81 (s, 1H), 6.44 (s, 1H), 4.97 (s, 1H), 4.44 (q, J = 6.7 Hz, 4H), 4.18 (dd, J = 16.8, 5.8 Hz, 1H), 4.00 (dd, J = 16.7, 5.6 Hz, 1H), 3.86 (t, J = 5.8 Hz, IH), 3.84-3.49 (m, 1H), 2.15 (dt, J = 13.3, 6.3 Hz, 2H), 1.47 (s, 3H). LC/MS (ESI) m/z: 596 (M + H)⁺. RT (Method A): 1.47 min. |
| 272 | 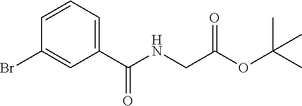 | 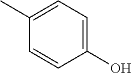 | 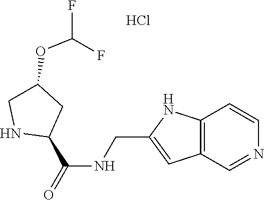 [SMILES: O=C(NCc1cc2cnccc2[nH]1)[C@@H]1C[C@@H](OC(F)F)CN1 ; Cl] | ¹H NMR (400 MHz, DMSO-d₆): δ 11.34 (s, 1H), 8.98-8.42 (m, 2H), 8.30- 7.88 (m, 1H), 7.63 (t, J = 9.0 Hz, 1H), 7.52-7.39 (m, 2H), 7.27-7.12 (m, 2H), 6.97-6.91 (m, 2H), 6.81 (s, 1H), 6.42 (s, 1H), 4.97 (s, 1H), 4.57-4.30 (m, 2H), 4.18 (dd, J = 16.8, 5,8 Hz, 2H), 4.01 (dd, J = 16.7, 5.7 Hz, 1H), 3.87 (dd, J = 11.7, 4.5 Hz, 1H), 3.83-3.68 (m, 1H), 2.30 (d, J = 3.8 Hz, 1H), 2.25-2.07 (m, 1H), 1.48 (s, 3H). LC/MS (ESI) m/z: 578 (M + H)⁺. RT (Method A): 1.50 min. |
| 273 | 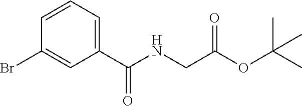 | 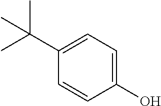 | 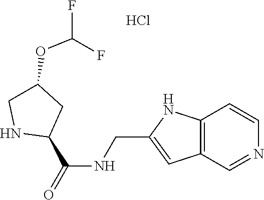 [SMILES: O=C(NCc1cc2cnccc2[nH]1)[C@@H]1C[C@@H](OC(F)F)CN1 ; Cl] | ¹H NMR (400 MHz, DMSO-d6): δ 11.24 (s, 1H), 8.89-8.74 (m, 1H), 8.69 (d, J = 13.5 Hz, 2H), 8.35 (s, 2H), 8.02 (d, J = 5.6 Hz, 1H), 7.65 (t, J = 9.2 Hz, 1H), 7.49 (q, J = 7.8 Hz, 2H), 7.18 (dd, J = 9.8, 6.6 Hz, 2H), 6.98 (t, J = 7.9 Hz, 3H), 6.81 (s, 1H), 4.97 (s, 1H), 4.57-4.33 (m, 3H), 4.19 (dd, J - 16.8, 5.8 Hz, 1H), 4.01 (dd, J = 16.8, 5.6 Hz, 1H), 3.87 (dd, J = 11.6, 4.5 Hz, 1H), 3.83-3.66 (m, 1H), 2.34 (s, 1H), 2.21-2.05 (m, 1H), 1.28 (d, J = 2.1 Hz, 9H). LC/MS (ESI) m/z: 620 (M + H)⁺. RT (Method A): 1.88 min. |
| 274 | 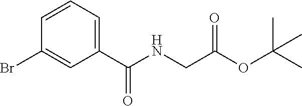 | 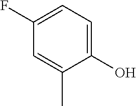 | 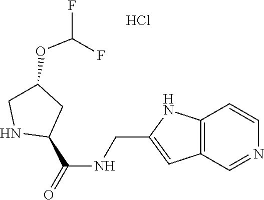 [SMILES: O=C(NCc1cc2cnccc2[nH]1)[C@@H]1C[C@@H](OC(F)F)CN1 ; Cl] | ¹H NMR (400 MHz, DMSO-d6): δ 11.39 (s, 1H), 9.02-8.45 (m, 2H), 8.40- 7.93 (m, 1H), 7.60 (t, J = 9.0 Hz, 1H), 7.47 (t, J = 7.9 Hz, 1H), 7.39-7.29 (m, 1H), 7.23 (dt, J = 9.1, 4.0 Hz, 2H), 7.08 (dd, J = 8.5, 2.8 Hz, 2H), 7.03- 6.94 (m, 1H), 6.80 (d, J = 14.6 Hz, 1H), 6.43 (s, 1H), 4.61-4.27 (m, 4H), 4.18 (dd, J = 16.7, 5.8 Hz, 1H), 4.01 (dd, J = 16.7, 5.6 Hz, 1H), 3.95-3.82 (m, 1H), 3.84-3.66 (m, 1H), 2.65 (d, J = 16.6 Hz, 1H), 2.33 (s, 3H), 2.16 (d, J = 5.2 Hz, IH), 1.70-1.39 (m, 1H). LC/MS (ESI) m/z: 596 (M + H)⁺. RT (Method A): 1.82 min. |
| 292 | 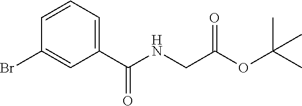 | 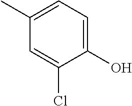 | 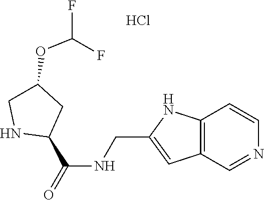 [SMILES: O=C(NCc1cc2cnccc2[nH]1)[C@@H]1C[C@@H](OC(F)F)CN1 ; Cl] | ¹H NMR (400 MHz, DMSO-d6): δ 11.45 (s, 1H), 9.04-8.44 (m, 4H), 8.13 (s, 0H), 7.63 (t, J = 8.7 Hz, 1H), 7.52-7.40 (m, 3H), 7.38 (t, J = 2.0 Hz, 1H), 7.34 (s, 0H), 7.19 (dd, J = 8.4, 2.0 Hz, 1H), 7.14-7.09 (m, 2H), 7.09 (s, 1H), 7.07-6.98 (m, 1H), 6.81 (s, 1H), 6.62 (s, 0H), 6.46 (s, 1H), 4.97 (s, 1H), 4.54-4.37 (m, 3H), 4.17 (dd, J = 16.8, 5.7 Hz, 1H), 4.07-3.95 (m, 1H), 3.88 (d, J = 11.7 Hz, 1H), 3.83-3.71 (m, 2H), 2.32 (d, J = 3.3 Hz, 5H), 2.18-2.10 (m, 1H), 1.47 (d, J = 9.0 Hz, 5H), 1.00-0.89 (m, 4H). LC/MS (ESI) m/z: 612 (M)⁺. RT (Method A): 1.70 min. |
| 293 | 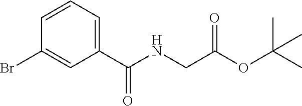 | 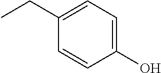 | 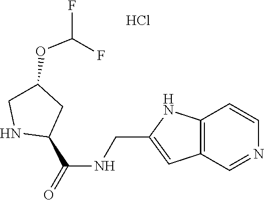 [SMILES: O=C(NCc1cc2cnccc2[nH]1)[C@@H]1C[C@@H](OC(F)F)CN1 ; Cl] | ¹H NMR (400 MHz, DMSO-d6): δ 11.22 (s, 1H), 8.81-8.71 (m, 1H), 8.67 (s, 2H), 8.36 (s, 1H), 8.02 (d, J = 5.6 Hz, 1H), 7.64 (t, J = 9.0 Hz, 1H), 7.53- 7.42 (m, 2H), 7.32-7.15 (m, 4H), 7.02-6.93 (m, 2H), 6.79 (d, J = 14.4 Hz, 1H), 6.38 (s, 1H), 4.97 (s, 1H), 4.54-4.36 (m, 3H), 4.18 (dd, J = 16.7, 5.8 Hz, 1H), 4.01 (dd, J = 16.8, 5.8 Hz, 1H), 3.87 (dd, J = 11.6, 4,6 Hz, 1H), 3.83-3.72 (m, 2H), 2.61 (q, J = 4.1 Hz, 1H), 2.61-2.52 (m, 1H), 2.20- 2.09 (m, 1H), 1.33 (s, 1H), 1.26-1.13 (m, 4H). LC/MS (ESI) m/z: 592 (M + H)⁺. RT (Method A): 1.59 min. |
| 294 | 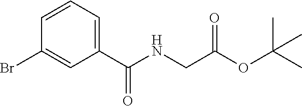 | 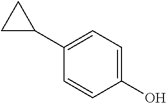 | 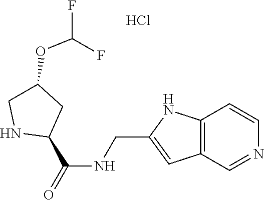 [SMILES: O=C(NCc1cc2cnccc2[nH]1)[C@@H]1C[C@@H](OC(F)F)CN1 ; Cl] | ¹H NMR (400 MHz, DMSO-d6): δ 11.25 (s, 1H), 9.12-8.39 (m, 2H), 8.16 (s, 2H), 7.64 (d, J = 8.0 Hz, 1H), 7.52-7.40 (m, 1H), 7.17 (tt, J = 9.2, 4.4 Hz, 1H), 7.10 (d, J = 8.1 Hz, 1H), 6.94 (dd, J = 8.6, 6.7 Hz, 3H), 6.79 (d, J = 14.5 Hz, 1H), 6.39 (s, 1H), 4.97 (s, 1H), 4.44 (t, J = 7.5 Hz, 3H), 4.18 (dd, J = 16.7, 5.6 Hz, 1H), 4.07-3.96 (m, 1H), 3.87 (s, 1H), 3.84-3.69 (m, 2H), 2.32 (t, J = 5.9 Hz, 2H), 2.16 (dd, J = 13.4, 6.8 Hz, 1H), 1.91 (ddt, J = 13.4, 8.7, 4.8 Hz, 1H), 1.51-1.39 (m, 1H), 1.01-0.83 (m, 2H), 0.72-0.53 (m, 2H). LC/MS (ESI) m/z: 604 (M + H)⁺. RT (Method A): 1.74 min. |
| 295 | 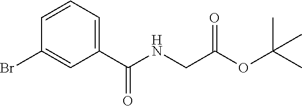 [SMILES: CC(C)(C)OC(=O)CNC(=O)c1cccc(Br)c1] | 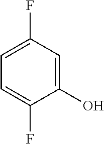 | 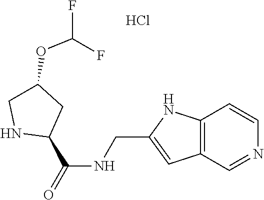 [SMILES: O=C(NCc1cc2cnccc2[nH]1)[C@@H]1C[C@@H](OC(F)F)CN1 ; Cl] | ¹H NMR (400 MHz, DMSO-d6): δ 12.17 (s, 1H), 8.81 (q, J = 4.8 Hz, 1H), 8.14 (s, 1H), 7.96 (t, J = 4.7 Hz, 1H), 7.69 (t, J = 8.0 Hz, 1H), 7.55-7.42 (m, 2H), 7.35-7.28 (m, 1H), 7.24 (dd, J = 8.2, 2.7 Hz, 1H), 7.14 (dddt, J = 11.9, 8.6, 5.4, 3.2 Hz, 1H), 7.06-6.91 (m, 1H), 6.80 (s, 1H), 6.61 (s, 1H), 6.54 (s, 1H), 4.95 (q, J - 4.1 Hz, 1H), 4.53 (1, J - 6.8 Hz, 1H), 4.51-4.35 (m, 1H), 4.20 (dd, J = 16.8, 5.8 Hz, 1H), 4.11-3.96 (m, 1H), 3.88 (dd, J = 11.6, 4.7 Hz, 1H), 3.84-3.63 (m, 1H), 2.32 (dtd, J = 15.5, 10.2, 5.3 Hz, 2H), 2.14 (ddd, J = 13.1, 7.4, 5.0 Hz, 1H), 1.67-1.35 (m, 1H). LC/MS (ESI) m/z: 600 (M + H)⁺. RT (Method A): 1.45 min |
| 298 | 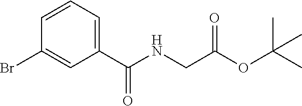 | 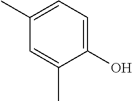 | 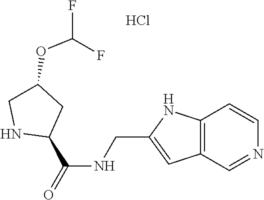 [SMILES: O=C(NCc1cc2cnccc2[nH]1)[C@@H]1C[C@@H](OC(F)F)CN1 ; Cl] | ¹H NMR (400 MHz, DMSO-d6): δ 11.21 (s, 1H), 8.68 (t, J = 12.8 Hz, 1H), 8.21 (s, 1H), 8.12-7.77 (m, 2H), 7.58 (d, J = 8.0 Hz, 1H), 7.44 (t, J = 7.9 Hz, 1H), 7.34 (d, J = 2.1 Hz, 1H), 7.32-7.25 (m, 1H), 7.16 (dd, J = 13.7, 3.8 Hz, 2H), 7.04 (td, J = 8.2, 2.4 Hz, 2H), 6.86 (t, J = 8.2 Hz, 1H), 6.80 (s, 1H), 6.38 (s, 1H), 4.97 (s, 1H), 4.43 (t, J = 7.5 Hz, 2H), 4.17 (dd, J = 16.7, 5.7 Hz, 1H), 4.00 (dd, J = 16.6, 5.5 Hz, 1H), 3.87 (dd, J = 11.7, 4.5 Hz, 1H), 3.81-3.70 (m, 2H), 2.28 (s, 3H), 2.13 (d, J = 14.6 Hz, 1H), 2.07 (s, 3H), 2.09 (d, J = 13.9 Hz, 1H). LC/MS (ESI) m/z: 592 (M + H)⁺. RT (Method A): 1.65 min. |
| 299 | 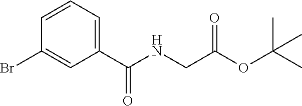 | 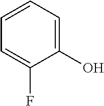 | 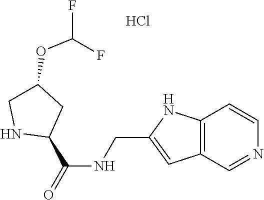 [SMILES: O=C(NCc1cc2cnccc2[nH]1)[C@@H]1C[C@@H](OC(F)F)CN1 ; Cl] | ¹H NMR (400 MHz, DMSO-d6): δ 11.15 (s, 1H), 8.75-8.61 (m, 1H), 8.63- 8.54 (m, 1H), 8.16 (s, 1H), 8.03 (d, J = 5.6 Hz, 1H), 7.96 (d, J = 5.7 Hz, 1H), 7.82 (td, J = 16.0, 7.5 Hz, 1H), 7.59 (t, J = 8.5 Hz, 1H), 7.51-7.28 (m, 1H), 7.25-7.08 (m, 1H), 6.72 (d, J = 14.6 Hz, 1H), 6.53 (d, J = 14.6 Hz, 1H), 4.90 (p, J = 4.0 Hz, 1H), 4.81 (d, J = 5.2 Hz, 1H), 4.61 (t, J = 7.5 Hz, 1H), 4.43 (d, J = 10.3 Hz, 1H), 4.37 (t, J = 7.1 Hz, 2H), 4.12 (dd, J = 16.7, 5.9 Hz, 2H), 4.02 (d, J = 14.0 Hz, 1H), 3.94 (dd, J = 16.6, 5.6 Hz, 1H), 3.85- 3.62 (m, 1H), 3.52 (dd, J = 12.7, 4.9 Hz, 1H), 2.24 (qd, J = 12.3, 4.4 Hz, 1H), 2.08 (ddd, J = 13.0, 7.5, 4.8 Hz, 1H). LC/MS (ESI) m/z: 582 (M + H)⁺. |

### Scheme 62. Synthesis of (2S,4R)-4-((1H-1,2,3-triazol-1-yl)methyl)-N-((R)-1-(1H-pyrrolo[3,2-c]pyridin-2-yl)ethyl)-4-fluoro-1-((phenoxathiine-3-carbonyl)glycyl)pyrrolidine-2-carboxamide (Compound 216)

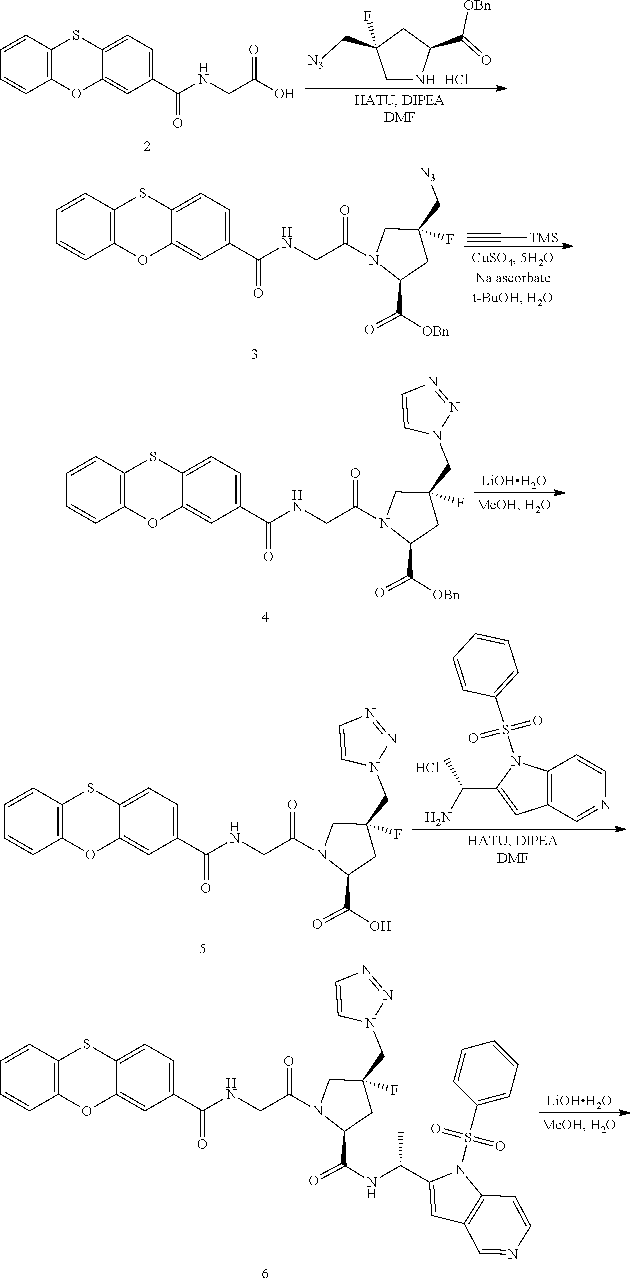

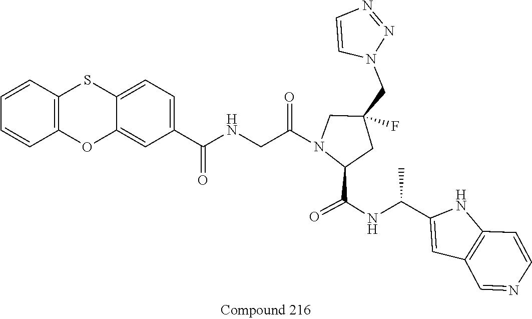 [SMILES: C[C@@H](NC(=O)[C@@H]1C[C@](F)(Cn2ccnn2)CN1C(=O)CNC(=O)c1ccc2c(c1)Oc1ccccc1S2)c1cc2cnccc2[nH]1]

### Step 1: Benzyl (2S,4R)-4-(azidomethyl)-4-fluoro-1-((phenoxathiine-3-carbonyl)glycyl) pyrrolidine-2-carboxylate (2)

To a mixture of (phenoxathiine-3-carbonyl)glycine (113 mg, 0.38 mol) and benzyl (2S,4R)-4-(azidomethyl)-4-fluoropyrrolidine-2-carboxylate hydrochloride (100 mg, 0.31 mmol) in DMF (3 mL) was added DIPEA (219 mg, 1.70 mmol) and HATU (194 mg, 0.51 mol) under N₂ atmosphere and the reaction mixture was stirred at room temperature for 1 hour. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-30% EtOAc in PE) to give the title compound (160 mg, yield 93.0%) as a yellow oil. LC/MS (ESI) m/z: 562 (M+H)⁺.

### Step 2: Benzyl (2S,4R)-4-((1H-1,2,3-triazol-1-yl)methyl)-4-fluoro-1-((phenoxathiine-3-carbonyl)glycyl)pyrrolidine-2-carboxylate (3)

To a solution of benzyl (2S,4R)-4-(azidomethyl)-4-fluoro-1-((phenoxathiine-3-carbonyl)glycyl)pyrrolidine-2-carboxylate (160 mg, 0.28 mmol) in t-BuOH (2.8 mL) and water (5.6 mL) was added trimethylsilylacetylene (56 mg, 0.57 mmol), CuSO₄·5H₂O (154 mg, 0.57 mmol) and Sodium ascorbate (320 mg, 1.62 mmol) at 0° C. The mixture was stirred in a sealed tube at 60° C. for 4 hours. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=15:1) to give the title compound (48 mg, yield 33.5%) as a white solid. ¹H NMR (400 MHz, CDCl₃) δ 7.77 (d, J=8.9 Hz, 1H), 7.67 (d, J=5.8 Hz, 1H), 7.44 (dd, J=4.2, 2.4 Hz, 1H), 7.38 (d, J=10.7 Hz, 6H), 7.24-7.21 (m, 1H), 7.15-7.11 (m, 1H), 7.10-6.96 (m, 3H), 5.45-5.12 (m, 3H), 4.86-4.62 (m, 3H), 4.26-3.88 (m, 2H), 3.79-3.58 (m, 1H), 2.58-2.42 (m, 1H), 2.29-2.17 (m, 1H). LC/MS (ESI) m/z: 588 (M+H)⁺.

### Step 3: (2S,4R)-4-((1H-1,2,3-triazol-1-yl)methyl)-4-fluoro-1-((phenoxathiine-3-carbonyl)glycyl)pyrrolidine-2-carboxylic acid (4)

To a solution of benzyl (2S,4R)-4-((1H-1,2,3-triazol-1-yl)methyl)-4-fluoro-1-((phenoxathiine-3-carbonyl)glycyl)pyrrolidine-2-carboxylate (45 mg, 0.077 mmol) in MeOH (2 mL) and water (1 mL) was added LiOH·H₂O (12 mg, 0.31 mmol) and the mixture was stirred at room temperature for 1 hour. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (34 mg, yield 89.5%) as a light-yellow oil, which was used directly in the next step without further purification. LC/MS (ESI) m/z: 498 (M+H)⁺.

### Step 4: (2S,4R)-4-((1H-1,2,3-triazol-1-yl)methyl)-4-fluoro-1-((phenoxathiine-3-carbonyl)glycyl)-N-((R)-1-(1-(phenylsulfonyl)-1H-pyrrolo[3,2-c]pyridin-2-yl)ethyl) pyrrolidine-2-carboxamide (5)

To a mixture of (2S,4R)-4-((1H-1,2,3-triazol-1-yl)methyl)-4-fluoro-1-((phenoxathiine-3-carbonyl)glycyl)pyrrolidine-2-carboxylic acid (34 mg, 0.068 mol) and (R)-1-(1-(phenylsulfonyl)-1H-pyrrolo[3,2-c]pyridin-2-yl)ethan-1-amine hydrochloride (34 mg, 0.10 mmol) in DMF (3 mL) was added DIPEA (44 mg, 0.34 mmol) and HATU (39 mg, 0.10 mol) under N₂ atmosphere and the reaction mixture was stirred at room temperature for 1 hour. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=15:1) to give the title compound (30 mg, yield 56.6%) as a white solid. LC/MS (ESI) m/z: 781 (M+H)⁺.

### Step 5: (2S,4R)-4-((1H-1,2,3-triazol-1-yl)methyl)-N-((R)-1-(1H-pyrrolo[3,2-c]pyridin-2-yl)ethyl)-4-fluoro-1-((phenoxathiine-3-carbonyl)glycyl)pyrrolidine-2-carboxamide (Compound 216)

To a solution of (2S,4R)-4-((1H-1,2,3-triazol-1-yl)methyl)-4-fluoro-1-((phenoxathiine-3-carbonyl)glycyl)-N-((R)-1-(1-(phenylsulfonyl)-1H-pyrrolo[3,2-c]pyridin-2-yl)ethyl) pyrrolidine-2-carboxamide (30 mg, 0.038 mol) in MeOH (2 mL) and water (1 mL) was added LiOH·H₂O (6 mg, 0.14 mol), and the mixture was stirred at 30° C. for 4 hours. The mixture was diluted with EtOAc, washed water and brine, dried over anhydrous Na₂SO₄, filtered, and concentrated under reduced pressure to dryness. The residue was purified by prep-HPLC to give Compound 216 (2.9 mg, yield 12.2%) as a white solid. ¹H NMR (400 MHz, CD₃OD): δ 8.64 (d, J=1.5 Hz, 1H), 8.08 (s, 1H), 7.88 (d, J=5.8 Hz, 1H), 7.80 (s, 1H), 7.60-7.55 (m, 2H), 7.26 (dd, J=8.1, 4.3 Hz, 1H), 7.21-7.15 (m, 2H), 7.08 (t, J=7.3 Hz, 2H), 7.03 (dd, J=8.1, 1.2 Hz, 1H), 6.52 (d, J=0.7 Hz, 1H), 5.35-5.29 (m, 1H), 5.07 (s, 1H), 5.02 (s, 1H), 4.62-4.57 (m, 1H), 4.16 (ddd, J=41.7, 21.8, 14.6 Hz, 3H), 3.98 (dd, J=19.2, 12.3 Hz, 1H), 2.47-2.43 (m, 1H), 2.42-2.30 (m, 1H), 1.60 (d, J=6.9 Hz, 3H). LC/MS (ESI) m/z: 641 (M+H)⁺. RT (Method A): 1.32 min.

### Scheme 63. Synthesis of (2S,4S)-4-((1H-imidazol-1-yl)methyl)-N-((R)-1-(1H-pyrrolo[3,2-e]pyridin-2-yl)ethyl)-4-fluoro-1-((phenoxathiine-3-carbonyl)glycyl)pyrrolidine-2-carboxamide (Compound 217)

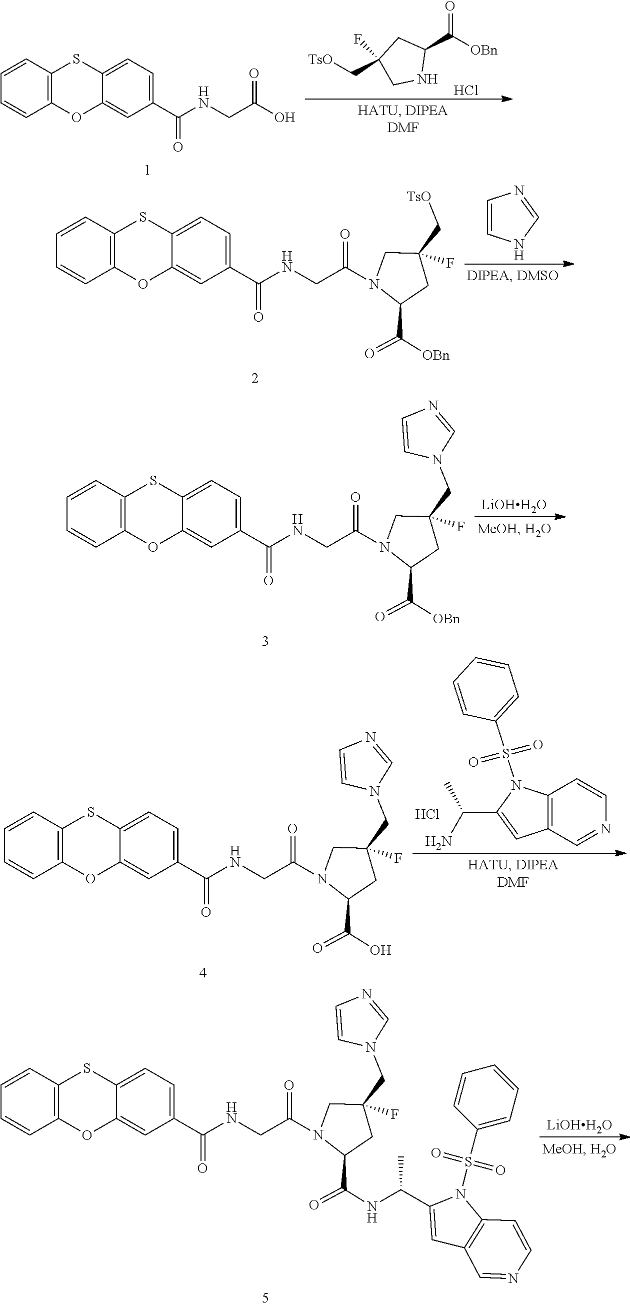

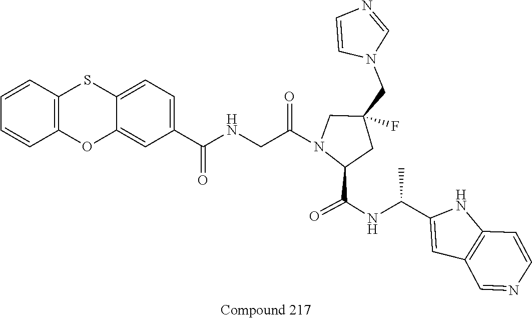 [SMILES: C[C@@H](NC(=O)[C@@H]1C[C@](F)(Cn2ccnc2)CN1C(=O)CNC(=O)c1ccc2c(c1)Oc1ccccc1S2)c1cc2cnccc2[nH]1]

### Step 1: Benzyl (2S,4R)-4-fluoro-1-((phenoxathiine-3-carbonyl)glycyl)-4-((tosyloxy) methyl)pyrrolidine-2-carboxylate(2)

To a mixture of benzyl (2S,4R)-4-fluoro-4-((tosyloxy)methyl)pyrrolidine-2-carboxylate hydrochloride (480 mg, 1.08 mmol) and (phenoxathiine-3-carbonyl)glycine (326 mg, 1.08 mmol) in DMF (5 mL) was added DIPEA (697 mg, 5.40 mmol) and HAI L (615 mg, 1.62 mol) under N₂ atmosphere and the reaction mixture was stirred at room temperature for 1 hour. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-40% EtOAc in PE) to give the title compound (740 mg, yield 99.0%) as a yellow oil. LC/MS (ESI) m/z: 691 (M+H)⁺.

### Step 2: Benzyl (2S,4S)-4-((1H-imidazol-1-yl)methyl)-4-fluoro-1-((phenoxathiine-3-carbonyl)glycyl)pyrrolidine-2-carboxylate (3)

To a solution of benzyl (2S,4R)-4-fluoro-1-((phenoxathiine-3-carbonyl)glycyl)-4-((tosyloxy)methyl)pyrrolidine-2-carboxylate (740 mg, 1.07 mmol) in DMSO (10 mL) was added 1II-imidazole (365 mg, 5.36 mmol) and DIPEA (1.38 g, 10.72 mmol) and the mixture was stirred at 120° C. for 20 hours. The reaction mixture was diluted with EtOAc, washed with water and brine, dried over anhydrous Na₂SO₄, filtered, and concentrated under reduced pressure to dryness. The residue was purified by flash chromatography (silica gel, 0-70% EtOAc in PE) to give the title compound (90 mg, yield 14.3%) as a yellow solid. LC/MS (ESI) n/z: 587 (M+H)⁺.

### Step 3: (2S,4S)-4-((1H-imidazol-1-yl)methyl)-4-fluoro-1-((phenoxathiine-3-carbonyl) glycyl)pyrrolidine-2-carboxylic acid (4)

To a solution of benzyl (2S,4S)-4-((1H-imidazol-1-yl)methyl)-4-fluoro-1-((phenoxathiine-3-carbonyl)glycyl)pyrrolidine-2-carboxylate (90 mg, 0.15 mmol) in MeOH (4 mL) and water (2 mL) was added LiOH·H₂O (26 mg, 0.61 mmol) and the mixture was stirred at room temperature for 2 hours. The mixture was acidified with IN aq. HCl to pH˜3 and extracted with EtOAc twice. The combined organic layers were washed with brine, dried over anhydrous Na₂SO₄, filtered, and concentrated under reduced pressure to dryness to give the title compound (40 mg, yield 52.6%) as a yellow oil, which was used directly in the next step without further purification. LC/MS (ESI) m/z: 497 (M+H)⁺.

### Step 4: (2S,4S)-4-((1H-imidazol-1-yl)methyl)-4-fluoro-1-((phenoxathiine-3-carbonyl)glycyl)-N-((R)-1-(1-(phenylsulfonyl)-1H-pyrrolo[3,2-c]pyridin-2-yl)ethyl)pyrrolidine-2-carboxamide (5)

To a mixture of (2S,4S)-4-((1H-imidazol-1-yl)methyl)-4-fluoro-1-((phenoxathiine-3-carbonyl)glycyl)pyrrolidine-2-carboxylic acid (40 mg, 0.081 mol) and (R)-1-(1-(phenylsulfonyl)-1H-pyrrolo[3,2-c]pyridin-2-yl)ethan-1-amine hydrochloride (41 mg, 0.12 mmol) in DMF (3 mL) was added DIPEA (52 mg, 0.41 mmol) and HATU (46 mg, 0.12 mol) under N₂ atmosphere and the reaction mixture was stirred at room temperature for 1 hour. The mixture was diluted with EtOAc, washed with saturated aq. NH₄Cl solution and brine, dried over anhydrous Na₂SO₄, filtered, and concentrated under reduced pressure to dryness. The residue was purified by prep-TLC (DCM:MeOH=15:1) to give the title compound (40 mg, yield 63.7%) as a white solid. L C/M S (ESI) m/z: 780 (M+H)⁺.

### Step 5: (2S,4S)-4-((1H-imidazol-1-yl)methyl)-N-((R)-1-(1H-pyrrolo[3,2-c]pyridin-2-yl)ethyl)-4-fluoro-1-((phenoxathiine-3-carbonyl)glycyl)pyrrolidine-2-carboxamide (Compound 217)

To a solution of (2S,4S)-4-((1H-imidazol-1-yl)methyl)-4-fluoro-1-((phenoxathiine-3-carbonyl)glycyl)-N-((R)-1-(1-(phenylsulfonyl)-1H-pyrrolo[3,2-c]pyridin-2-yl)ethyl) pyrrolidine-2-carboxamide (40 mg, 0.051 mol) in MeOH (2 mL) and water (1 mL) was added LiOH·H₂O (8.6 mg, 0.21 mol), and the mixture was stirred at 30° C. for 4 hours. The mixture was diluted with EtOAc, washed water and brine, dried over anhydrous Na₂SO₄, filtered, and concentrated under reduced pressure to dryness. The residue was purified by prep-HPLC to give Compound 217 (6.7 mg, yield 20.4%) as a white solid. ¹H NMR (400 MHz, CD₃OD): δ 8.64 (s, 1H), 7.88 (d, J=5.8 Hz, 1H), 7.74 (s, 1H), 7.61-7.56 (m, 2H), 7.28 (d, J=8.0 Hz, 1H), 7.21 (d, J=7.6 Hz, 2H), 7.18 (s, 1H), 7.10-7.05 (m, 2H), 7.03 (d, J=7.0 Hz, 2H), 6.52 (s, 1H), 5.32 (t, J=6.9 Hz, 1H), 4.63 (s, 11), 4.60-4.55 (m, 2H). 4.34-4.11 (m, 2H), 4.05-3.93 (m, 2H), 2.41-2.35 (m, 1H₁), 2.34-2.19 (m, 1H), 1.60 (d, J=7.0 Hz, 3H). LC/MS (ESI) m/z: 580 (M+H)⁺. RT (Method A): 0.95 min.

### Scheme 64. Synthesis of (2S,4R)-4-(difluoromethoxy)-1-((phenoxathiine-3-carbonyl)glycyl)-N-((1,2,3,4-tetrahydroisoquinolin-6-yl)methyl)pyrrolidine-2-carboxamide (Compound 223)

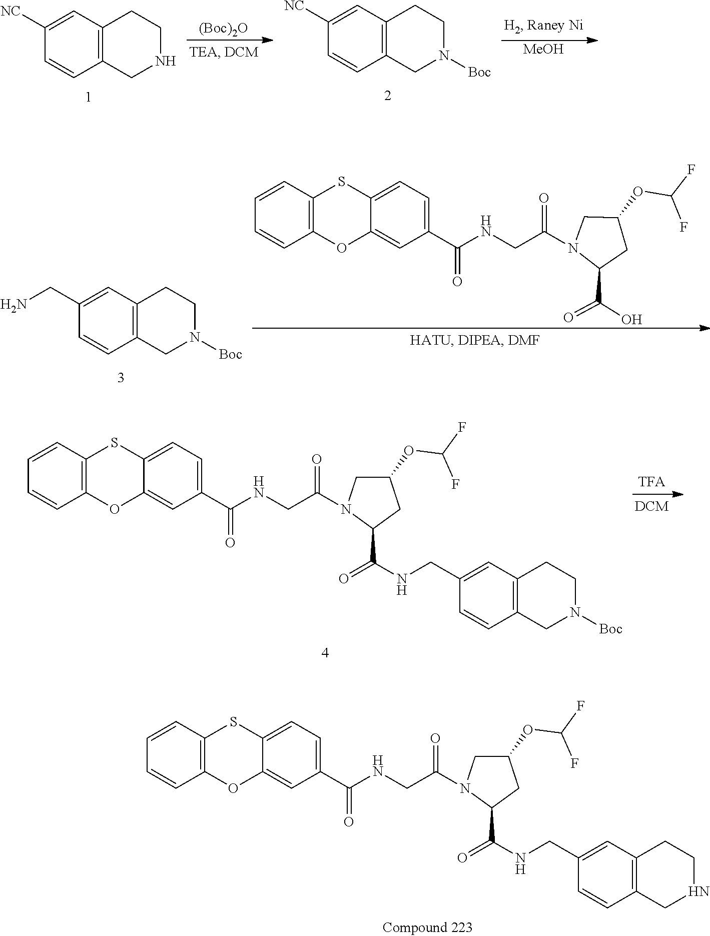

### Step 1: Tert-butyl 6-cyano-3,4-dihydroisoquinoline-2(III)-carboxylate (2)

To a solution of 1,2,3,4-tetrahydroisoquinoline-6-carbonitrile (120 mg, 0.76 mmol) in DCM (3 mL) was added (Boc)₂O (248 mg, 1.15 nmol) followed by TEA (0.21 mL, 1.52 mmol) at 0° C. and the mixture was stirred at room temperature for 6 hours. The mixture was diluted with water and extracted with DCM twice. The combined organic layers were washed with brine, dried over anhydrous Na₂SO₄, filtered, and concentrated to dryness. The residue was purified by flash chromatography (silica gel, 0-30% EtOAc in PE) to give the title compound (160 mg, yield 81.7%) as a white solid. LC/MS (ESI) m/z: 259 (M+H)⁺.

### Step 2: Tert-butyl 6-(aminomethyl)-3,4-dihydroisoquinoline-2(1H)-carboxylate (3)

To a solution of tert-butyl 6-cyano-3,4-dihydroisoquinoline-2(1H)-carboxylate (80 mg, 0.31 mmol) in MeOH (3 mL) was added Raney Ni (10 mg, wet), the mixture was degassed under N₂ atmosphere for three times and stirred under a H₂ balloon at room temperature for 3 hours. The mixture was filtered, and the filtrate was concentrated to dryness to give the title compound (65 mg, yield 80.0%) as a yellow oil, which was used directly in the next step without further purification. LC/MS (ESI) m/z: 263 (M+H)⁺.

### Step 3: Tert-butyl 6-(((2S,4R)-4-(difluoromethoxy)-1-((phenoxathiine-3-carbonyl) glycyl)pyrrolidine-2-carboxamido)methyl)-3,4-dihydroisoquinoline-2(1H)-carboxylate (4)

To a mixture of (2S,4R)-4-(difluoromethoxy)-1-((phenoxathiine-3-carbonyl)glycyl) pyrrolidine-2-carboxylic acid (35 mg, 0.08 mol) and tert-butyl 6-(aminomethyl)-3,4-dihydroisoquinoline-2(1H)-carboxylate (20 mg, 0.08 mmol) in DMF (2 mL) was added DIPEA (30 mg, 0.23 mmol) and HATU (43 mg, 0.11 mol) under N₂ atmosphere and the reaction mixture was stirred at room temperature for 2 hours. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-100% EtOAc in PE) to give the title compound (35 mg, yield 64.8%) as a yellow oil. LC/MS (ESI) m/z: 709 (M+H)⁺.

### Step 4: (2S,4R)-4-(difluoromethoxy)-1-((phenoxathiine-3-carbonyl)glycyl)-N-((1,2,3,4-tetrahydroisoquinolin-6-yl)methyl)pyrrolidine-2-carboxamide (Compound 223)

To a solution of tert-butyl 6-(((2S,4R)-4-(difluoromethoxy)-1-((phenoxathiine-3-carbonyl)glycyl)pyrrolidine-2-carboxamido)methyl)-3,4-dihydroisoquinoline-2(1H)-carboxylate (35 mg, 0.05 mmol) in 1,4-dioxane (1 mL) was added HCl/1,4-dioxane (0.5 mL, 4M) and the mixture was stirred at room temperature for 2 hours. The mixture was concentrated to dryness and the residue was purified by prep-HPLC to give Compound 223 (4.5 mg, yield 15.0%) as a white solid. ¹H NMR (400 MHz, DMSO-d6): δ 8.84-8.39 (m, 2H), 7.64-7.52 (m, 2H), 7.38 (dd, J=8.1, 2.3 Hz, 1H), 7.32-7.22 (m, 2H), 7.17-7.11 (m, 2H), 7.05-6.59 (m, 4H), 4.98-4.83 (m, 1H), 4.66-4.40 (m, 1H), 4.32-4.10 (m, 3H), 4.08-3.98 (m, 1H), 3.91-3.81 (m, 1H), 3.78 (s, 2H), 3.70-3.56 (m, 1H), 2.93-2.77 (m, 2H), 2.69-2.58 (m, 2H), 2.35-2.20 (m, 1H), 2.17-1.85 (m, 1H). LC/MS (ESI) m/z: 609 (M+H)⁺. RT (Method A): 1.50 min.

### Scheme 65. Synthesis of (2S,4R)-4-(difluoromethoxy)-1-((phenoxathiine-3-carbonyl)glycyl)-N-((1,2,3,4-tetrahydroisoquinolin-7-yl)methyl)pyrrolidine-2-carboxamide (Compound 226)

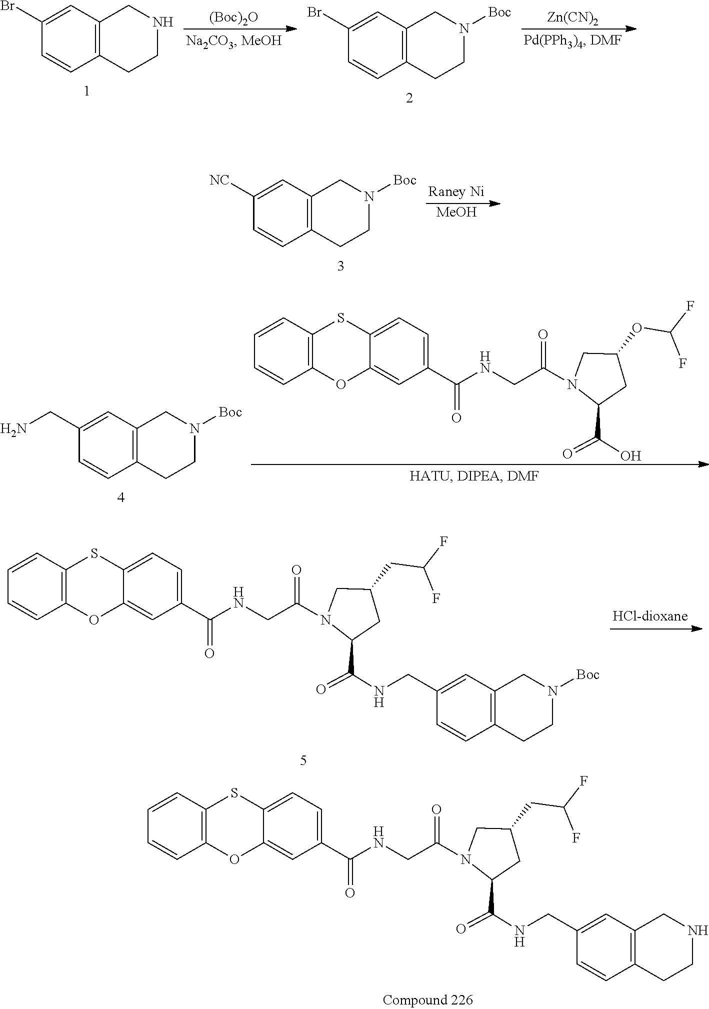

### Step 1: Tert-butyl 7-bromo-3,4-dihydroisoquinoline-2(1H)-carboxylate (2)

To a solution of 7-bromo-1,2,3,4-tetrahydroisoquinoline (1.5 g, 7.07 mmol) in MeOH (15 mL) was added 2N aq. Na₂CO₃ solution (21 mL, 42 mmol) and (Boc)₂O (1.7 g, 7.77 mmol) under N₂ atmosphere, the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-20% EtOAc in PE) to give the title compound (1.66 g, yield 75.1%) as a yellow solid. LC/MS (ESI) m/z: 312 (M+H)⁺.

### Step 2: Tert-butyl 7-cyano-3,4-dihydroisoquinoline-2(1H)-carboxylate (3)

To a mixture of tert-butyl 7-bromo-3,4-dihydroisoquinoline-2(1H)-carboxylate (1 g, 3.21 mmol) and Zn(CN)₂ (1.1 g, 0.32 mmol) in DMF (10 mL) was added Pd(PPh₃)₄ (370 mg, 0.32 mmol) under N₂ atmosphere, the reaction mixture was degassed under N₂ atmosphere for three times and stirred at 100° C. for 3 hours. The mixture was concentrated under reduced pressure to dryness. The residue was purified by flash chromatography (silica gel, 0-20% EtOAc in PE) to give the title compound (680 mg, yield 82.5%) as a yellow solid. ¹H NMR (400 MHz, DMSO): δ 7.70 (s, 1H), 7.61 (d, J=7.9 Hz, 1H), 7.37 (d, J=7.9 Hz, 1H), 4.54 (s, 2H), 3.56 (t, J=5.8 Hz, 2H), 2.85 (t, J=5.7 Hz, 2H), 1.43 (s, 9H). LC/MS (ESI) m/z: 259 (M+H)⁺.

### Step 3: Tert-butyl 7-(aminomethyl)-3,4-dihydroisoquinoline-2(1H)-carboxylate (4)

To a solution of tert-butyl 7-cyano-3,4-dihydroisoquinoline-2(1H)-carboxylate (100 mg, 0.38 mmol) in MeOH (2 mL) was added Raney Ni (10 mg, wet), the mixture was degassed under N₂ atmosphere for ten times and stirred under a H₂ balloon at room temperature overnight. The mixture was filtered, and the filtrate was concentrated under reduced pressure to dryness to give the title compound (80 mg, yield 79.2%) as a yellow solid. LC/MS (ESI) m/z: 263 (M+H)⁺.

### Step 4: Tert-butyl 7-(((2S,4R)-4-(difluoromethoxy)-1-((phenoxathiine-3-carbonyl) glycyl)pyrrolidine-2-carboxamido)methyl)-3,4-dihydroisoquinoline-2(1H)-carboxylate (5)

To a mixture of (2S,4R)-4-(difluoromethoxy)-1-((phenoxathiine-3-carbonyl)glycyl) pyrrolidine-2-carboxylic acid (30 mg, 0.064 mol) and tert-butyl 7-(aminomethyl)-3,4-dihydroisoquinoline-2(1H)-carboxylate (34 mg, 0.12 mmol) in DMF (1 mL) was added DIPEA (36 mg, 0.32 mmol) and HATU (41 mg, 0.10 mol) under N₂ atmosphere and the reaction mixture was stirred at room temperature for 30 mins. The mixture was diluted with EtOAc, washed with saturated aq. NH₄Cl solution and brine, dried over anhydrous Na₂SO₄, filtered, and concentrated under reduced pressure to dryness. The residue was purified by flash chromatography (silica gel, 0-80% EtOAc in PE) to give the title compound (30 mg, yield 66.6%) as a white solid. LC/MVS (ESI) m/z: 709 (M+H)⁺.

### Step 5: (2S,4R)-4-(Difluoromethoxy)-1-((phenoxathiine-3-carbonyl)glycyl)-N-((1,2,3,4-tetrahydroisoquinolin-7-yl)methyl)pyrrolidine-2-carboxamide (Compound 226)

To a solution of tert-butyl 7-(((2S,4R)-4-(difluoromethoxy)-1-((phenoxathiine-3-carbonyl)glycyl)pyrrolidine-2-carboxamido)methyl)-3,4-dihydroisoquinoline-2(1H)-carboxylate (30 mg, 0.04 mmol) in DCM (1 mL) was added TFA (0.5 mL) at 0° C. and the reaction mixture was stirred at room temperature for 2 hours. The mixture was concentrated under reduced pressure to dryness. The residue was purified by prep-HPLC to give Compound 226 (15.2 mg, yield 60.8%) as a white solid. ¹H NMR (400 MHz, CD₃OD): δ 7.55-7.42 (m, 2H), 7.24-7.15 (m, 4H), 7.11-7.00 (m, 4H), 6.51 (t, J=74.4 Hz, 1H), 5.04-4.98 (m, 1H), 4.60-4.58 (m, 11H), 4.39-4.37 (m, 2H), 4.21-4.15 (m, 3H), 4.01-3.94 (m, 1H), 3.86 (d, J=11.3 Hz, 1H), 3.43-3.38 (m, 3H), 3.04- 2.94 (m, 2H), 2.54-2.42 (m, 1H), 2.29-2.17 (m, 1H). LC/MS (ESI) m/z: 609 (M+H)⁺. RT (Method A): 1.50 min.

The following compounds were prepared based on Scheme 65:

| Cmpd # | Reactant A | Reactant B | Characterization Data |
|---|---|---|---|
| 253 | 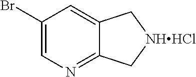 | 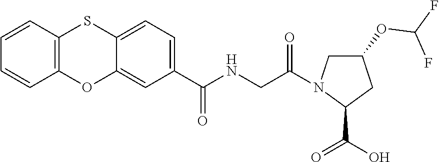 [SMILES: O=C(NCC(=O)N1C[C@H](OC(F)F)C[C@H]1C(=O)O)c1ccc2c(c1)Oc1ccccc1S2] | ¹H NMR (400 MHz, DMSO-d6): δ 11.21 (s, 1H), 8.65 (d, J = 14.2 Hz, 2H), 8.24 (d, J = 2.5 Hz, 1H), 8.02 (d, J = 5.4 Hz, 1H), 7.62 (t, J = 9.7 Hz, 1H), 7.42 (d, J = 7.7 Hz, 2H), 7.35 (d, J = 9.5 Hz, 1H), 6.83 (q, J = 9.7 Hz, 3H), 6.37 (s, 1H), 4.96 (s, 1H), 4.65 (d, J = 7.8 Hz, 1H), 4.52-4.25 (m, 3H), 4.15 (dd, J = 16.8, 5.6 Hz, 1H), 3.97 (d, J = 16.9 Hz, 1H), 3.82-3.66 (m, 1H), 2.26 (s, 3H), 2.15 (s, 3H). LC/MS (ESI) m/z: 592 (M + H)⁺. RT (Method A): 1.65 min. |
| 257^{a} | 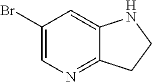 | 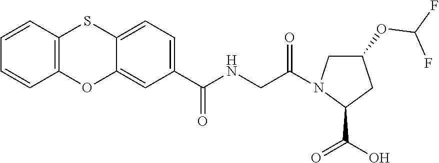 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 7.61 (d, J = 12.6 Hz, 1H), 7.52 (dd, J = 8.1, 1.8 Hz, 1H), 7.49 (dd, J = 7.3, 1.8 Hz, 1H), 7.24-7.17 (m, 2H), 7.15 (dd, J = 7.8, 1.6 Hz, 1H), 7.06 (dd, J = 15.0, 7.7 Hz, 2H), 6.87 (d, J = 15.8 Hz, 1H), 6.70- 6.28 (m, 1H), 5.02-4.97 (m, 1H), 4.56 (t, J = 7.8 Hz, 1H), 4.34-4.25 (m, 2H), 4.23-4.11 (m, 2H), 3.96 (dd, J = 11.4, 4.7 Hz, 1H), 3.85 (d, J = 12.1 Hz, 1H), 3.57-3.50 (m, 2H), 2.99 (t, J - 8.7 Hz, 2H), 2.51-2.42 (m, 1H), 2.26-2.17 (m, 1H). LC/MS (ESI) m/z: 596 (M + H)⁺. RT (Method A): 1.48 min. |
| 258^{b,c} | 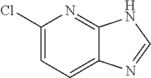 | 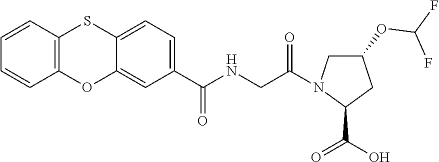 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 8.33 (d, J = 13.5 Hz, 1H), 7.95 (dd, J = 26.9, 8.3 Hz, 1H), 7.56-7.41 (m, 2H), 7.37-7.31 (m, 1H), 7.21-7.12 (m, 3H), 7.09- 7.00 (m, 2H), 6.71-6.29 (m, 1H), 5.05-4.94 (m, 1H), 4.70-4.61 (m, 3H), 4.27- 4.13 (m, 2H), 4.03-3.84 (m, 2H), 2.69-2.47 (m, 1H), 2.45-2.26 (m, 1H). LC/MS (ESI) m/z: 595 (M + H)⁺. RT (Method A): 1.70 min. |

^{a}Step 1 was performed in the presence of DIPEA in DCM.
^{b}Steps 2-4 only.
^{c}NMP was used as the solvent in Step 2.

### Scheme 66. Synthesis of (2S,4R)-N-((3-aminobenzo[d]isoxazol-6-yl)methyl)-4-(difluoromethoxy)-1-((phenoxathiine-3-carbonyl)glycyl)pyrrolidine-2-carboxamide (Compound 232)

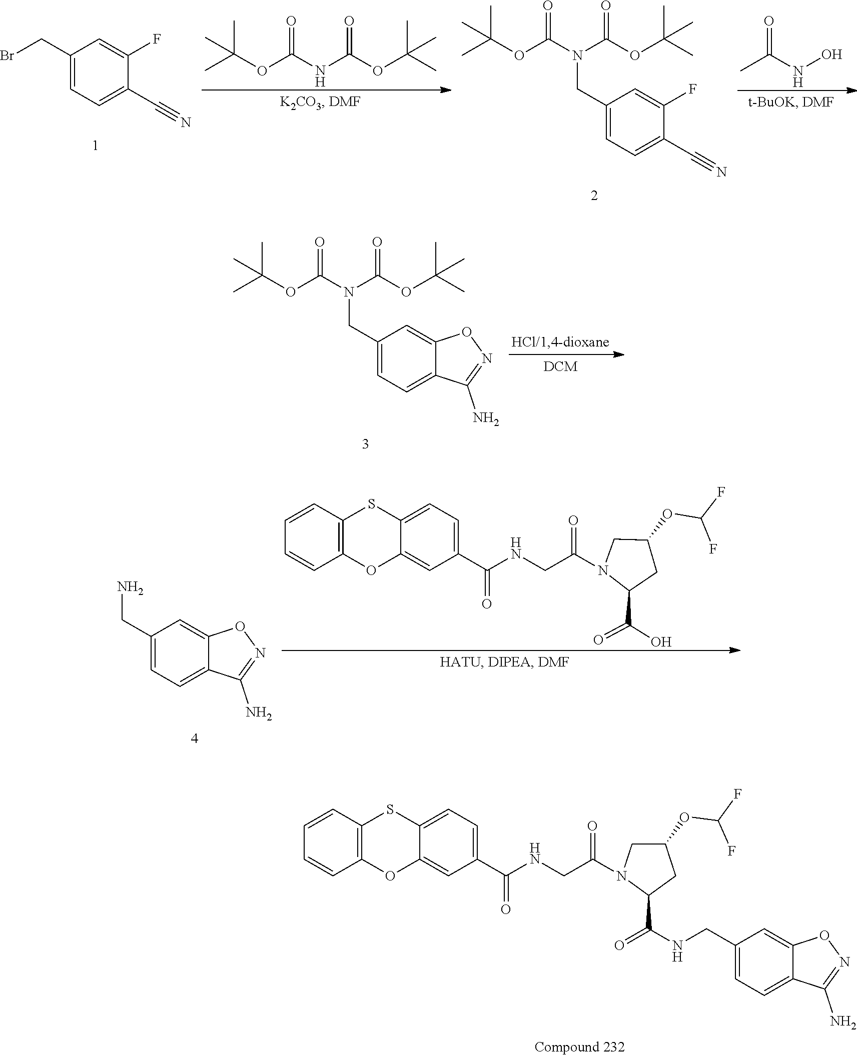

### Step 1: Tert-butyl (tert-butoxycarbonyl)(4-cyano-3-fluorobenzyl)carbamate (2)

To a solution of 4-(bromomethyl)-2-fluorobenzonitrile (500 mg, 2.33 mol) in DMF (5 mL) was added K₂CO₃ (970 mg, 7.02 mmol) and di-tert-butyl imidodicarbonate (760 mg, 3.49 mmol) under N₂ atmosphere and the mixture was stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% EtOAc in PE) to give the title compound (320 mg, yield 39.1%) as a white solid. LC/MS (ESI) (m/z): 351 (M+H)⁺.

### Step 2: Tert-butyl ((3-aminobenzo[d]isoxazol-6-yl)methyl)(tert-butoxycarbonyl) carbamate (3)

To a mixture of tert-butyl (tert-butoxy carbonyl)(4-cyano-3-fluorobenzyl)carbamate (270 mg, 0.77 mmol) and N-hydroxyacetamide (87 mg, 1.16 mmol) in DMF (3 mL) was added t-BuOK (1.15 mL, 1.15 mmol, 1 M in THF) and the mixture was stirred at room temperature for 1 hour. The mixture was extracted with EtOAc twice. The combined organic layers were washed with brine, dried over anhydrous Na₂SO₄, filtered, and concentrated under reduced pressure to dryness. The residue was purified by flash chromatography (silica gel, 0-70% EtOAc in PE) to give the title compound (120 mg, yield 99.5%) as a white solid. LC/MS (ESI) m/z: 393 (M+H)⁺.

### Step 3: 6-(Aminomethyl)benzo[d]isoxazol-3-amine (4)

To a solution of tert-butyl ((3-aminobenzo[d]isoxazol-6-yl)methyl)(tert-butoxycarbonyl)carbamate (40 mg. 0.11 mmol) in DCM (1 mL) was added HCl/1,4-dioxane (1 mL, 4 M) at 0° C. and the mixture was stirred under N₂ atmosphere at room temperature for 2 hours. The reaction mixture was concentrated under reduced pressure to dryness to give the title compound (17 mg, yield 94.6%) as a white solid, which was used directly in the next step without further purification. LC/MS (ESI) m/z: 164 (M+H)⁺.

### Step 4: (2S,4R)-N-((3-aminobenzo[d]isoxazol-6yl)methyl)-4-(difluoromethoxy)-1-((phenoxathiine-3-carbonyl)glycyl)pyrrolidine-2-carboxamide (Compound 232)

To a solution of (2S,4R)-4-(difluoromethoxy)-1-((phenoxathiine-3-carbonyl)glycyl) pyrrolidine-2-carboxylic acid (50 mg, 0.11 mol) and 6-(aminomethyl)benzo [d]isoxazol-3-amine (17 mg, 0.11 mmol) in DMF (2 mL) was added DIPEA (72 mg, 0.55 mmol) and HATU (54 mg, 0.14 mol) under N₂ atmosphere and the reaction mixture was stirred at room temperature for 1 hour. The mixture was concentrated under reduced pressure to dryness. The residue was purified by prep-HPLC to give Compound 232 (5.8 mg, yield 8.6%) as a white solid. ¹H NMR (400 MHz, CD₃OD): δ 7.68 (dd, J=22.2, 8.3 Hz, 1H), 7.54-7.45 (m, 2H), 7.36 (s, 1H), 7.23-7.17 (m, 3H), 7.14 (d, J=7.7 Hz, 1H), 7.06 (dd, J=14.3, 7.8 Hz, 2H), 6.51 (t, J=74.5 Hz, 1H), 5.01 (s, 1H), 4.64-4.52 (m, 3H), 4.28-4.12 (m, 2H), 3.96 (dd, J=114, 4.5 Hz, 1H), 3.89-3.81 (m, 1H), 2.61-2.41 (m, 1H), 2.30-2.15 (m, 1H). LC/MS (ESI) m/z: 610 (M+H)⁺. RT (Method A): 2.00 min.

### Scheme 67. Synthesis of (S)-N-((R)-1-(1H-pyrrolo[3,2-c]pyridin-2-yl)ethyl)-2-((phenoxathiine-3-carbonyl)glycyl)-2-azaspiro[4.5]decane-3-carboxamide (Compound 234)

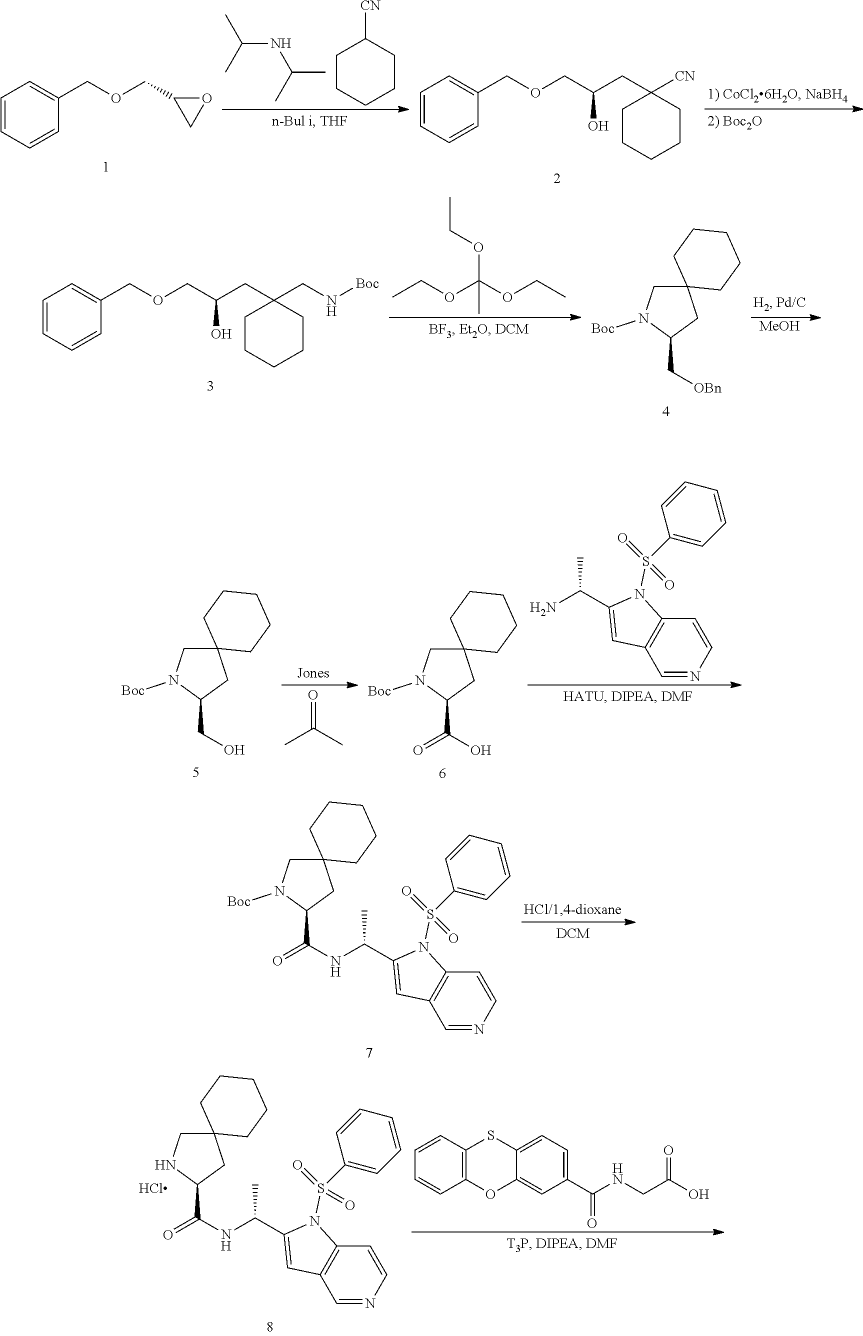

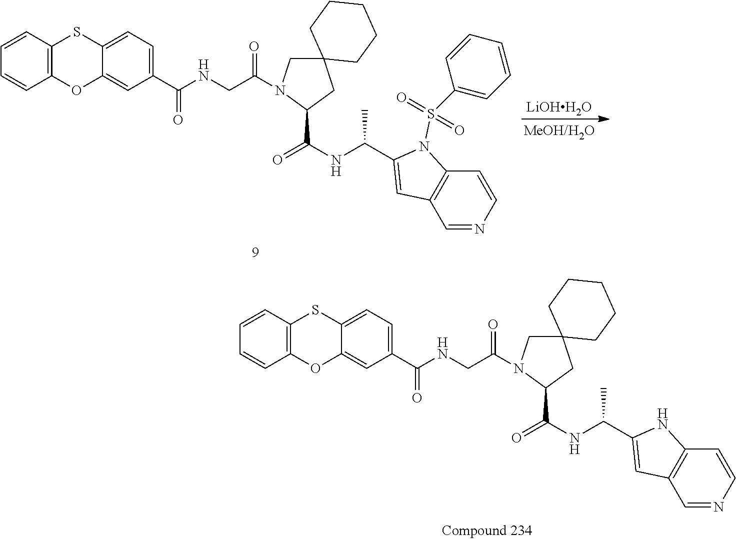

### Step 1: (R)-1-(3-(benzyloxy)-2-hydroxypropyl)cyclohexane-1-carbonitrile (2)

To a solution of diisopropylamine (1.35 g, 13.4 mmol) in anhydrous THF (20 mL) was added n-BuLi (4.8 mL, 12.2 mmol, 2.5M in Hexanes) drop-wisely at −78° C. under N₂ atmosphere and the mixture was stirred at this temperature for 30 mins. A solution of cyclohexanecarbonitrile (666 mg, 6.1 mmol) in anhydrous THE (10 mL) was added to the above mixture drop-wisely at −78° C. under N₂ atmosphere and the mixture was stirred at −78° C. for 1 hour. To the above mixture, a solution of (R)-2-((benzyloxy)methyl)oxirane (500 mg, 3.05 mmol) in THF (5 mL) was added drop-wisely at −78° C. under N₂ atmosphere and the resulting mixture was stirred at −78° C. to 20° C. for 20 mins. The reaction mixture was quenched with saturated aq. NH₄Cl solution at 0° C. and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (681 mg, yield 81.9%) as a colorless oil. LC/MS (EST) m/z: 274 (M+H)⁺.

### Step 2: Tert-butyl (R)-((1-(3-(benzyloxy)-2-hydroxypropyl)cyclohexyl)methyl) carbamate (3)

To a mixture of (R)-1-(3-(benzyloxy)-2-hydroxypropyl)cyclohexane-1-carbonitrile (580 mg, 2.13 mmol) and CoCl₂·6H₂O (1.01 g, 4.25 mmol) in MeOH (30 mL) was added NaBH₄ (404 mg, 10.63 mmol) in portion at 0° C. and the mixture was stirred at room temperature for 1 hour. The reaction mixture was quenched with 1 N aq. HCl, and the mixture was concentrated under reduced pressure to remove MeOH. The aqueous mixture was basified with IN aq. NaOH and a solution of Boc₂O (926 mg, 4.25 mmol) in DCM (10 mL) was added. The resulting mixture was stirred at room temperature overnight. The mixture was diluted with DCM, washed with water and brine, dried over anhydrous Na₂SO₄, filtered, and concentrated under reduced pressure to dryness. The residue was purified by flash chromatography (silica gel, 0-30% EtOAc in PE) to give the title compound (550 mg, yield 68.5%) as a colorless oil. LC/MS (ESI) m/z: 378 (M+H)⁺.

### Step 3: Tert-butyl (S)-3-((benzyloxy)methyl)-2-azaspiro[4.5]decane-2-carboxylate (4)

To a mixture of tert-butyl (R)-((1-(3-(benzyloxy)-2-hydroxypropyl)cyclohexyl) methyl)carbamate (550 mg, 1.46 mmol) and 1,1,1-triethoxyethane (355 mg, 2.19 mmol) in DCM (6 mL) was added BF₃·Et₂O (104 mg, 0.73 mmol) at 0° C. and the mixture was stirred at room temperature for 2 hours. The reaction mixture was quenched with saturated aq. NaHCO₃ solution, extracted with DCM twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (180 mg, yield 34.3%) as a colorless oil. LC/MS (ESI) m/z: 360 (M+H)⁺.

### Step 4: Tert-butyl (S)-3-(hydroxymethyl)-2-azaspiro[4.5]decane-2-carboxylate (5)

To a solution of tert-butyl (S)-3-((benzyloxy)methyl)-2-azaspiro[4.5]decane-2-carboxylate (180 mg, 0.50 mmol) in MeOH (2 mL) was added Pd/C (20 mg, 10% wt.) and the mixture was stirred under a H₂ balloon at room temperature for 2 hours. The mixture was filtered, and the filtrate was concentrated to dryness to give the title compound (130 mg, yield 96.3%) as a colorless oil, which was used directly in the next step without further purification. LC/MS (ESI) m/z: 270 (M+H)⁺.

### Step 5: (S)-2-(tert-butoxycarbonyl)-2-azaspiro[4.5]decane-3-carboxylic acid (6)

To a solution of tert-butyl (S)-3-(hydroxymethyl)-2-azaspiro[4.5]decane-2-carboxylate (130 mg, 0.48 mmol) in acetone (3 mL) was added Jones oxidant (15 drops, 2.7 M, newly prepared) under N₂ atmosphere at room temperature and the mixture was stirred at room temperature for 10 minutes. The mixture was filtered. The filtrate was diluted with EtOAc, washed with water and brine, dried over anhydrous Na₂SO₄, filtered, and concentrated under reduced pressure to dryness to give the title compound (64 mug, yield 47.1%) as a colorless oil, which was used directly in the next step without further purification. LC/MS (ESI) m/z: 284 (M+H)⁺.

### Step 6: Tert-butyl (S)-3-(((R)-1-(1-(phenylsulfonyl)-JH-pyrrolo[3,2-c]pyridin-2-yl)ethyl)carbamoyl)-2-azaspiro[4.5]decane-2-carboxylate (7)

To a mixture of (S)-2-(tert-butoxycarbonyl)-2-azaspiro[4.5]decane-3-carboxylic acid (30 mg, 0.11 mmol) and (R)-1-(1-(phenylsulfonyl)-1H-pyrrolo[3,2-c]pyridin-2-yl)ethan-1-amine hydrochloride (48 mg. 0.16 mmol) in DMF (1 mL) was added DIPEA (68 mg, 0.53 mmol) and HATU (60 mg, 0.16 mmol) under N₂ atmosphere at 0° C. and the mixture was stirred at room temperature for 1 hour. The mixture was concentrated under reduced pressure to dryness. The residue was purified by prep-TLC (DCM:MeOH=8:1) to give the title compound (25 mg, yield 41.7%) as a white solid. LC/MS (ESI) (m/z): 567 (M+H)⁺.

### Step 7: (S)-N-((R)-1-(1-(phenylsulfonyl)-H-pyrrolo[3,2-c]pyridin-2-yl)ethyl)-2-azaspiro[4.5]decane-3-carboxamide hydrochloride (8)

A solution of tert-butyl (S)-3-(((R)-1-(1-(phenylsulfonyl)-1H-pyrrolo[3,2-c]pyridin-2-yl)ethyl)carbamoyl)-2-azaspiro[4.5]decane-2-carboxylate (25 mg, 0.044 mmol) in HCl/1,4-dioxane (1 mL, 4M) was stirred under N₂ atmosphere at room temperature for 2 hours. The reaction mixture was concentrated under reduced pressure to dryness to give the title compound (21 mg, yield 94.6%) as a white solid, which was used directly in the next step without further purification. LC/MS (ESI) m/z: 467 (M+H)⁺.

### Step 8: GS)-2-((phenoxathiine-3-carbonyl)glycyl)-N-((R)-1-(1-(phenylsulfonyl)-1H-pyrrolo[3,2-c]pyridin-2-yl)ethyl)-2-azaspiro[4.5]decane-3-carboxamide (9)

To a mixture of (phenoxathiine-3-carbonyl)glycine (15 mg, 0.049 mmol) and (S)-N-((R)-1-(1-(phenylsulfonyl)-1H-pyrrolo[3,2-c]pyridin-2-yl)ethyl)-2-azaspiro[4.5]decane-3-carboxamide hydrochloride (21 mg, 0.044 mmol) in DMF (1 mL) was added DIPEA (29 mg, 0.22 mmol) and T₃P (42 mg, 0.066 mmol, 50% in EtOAc wt.) under N₂ atmosphere at 0° C. and the mixture was stirred at room temperature for 1 hour. The mixture was concentrated under reduced pressure to dryness. The residue was purified by prep-TLC (DCM:MeOH=10:1) to give the title compound (23 mg, yield 63.8%) as a white solid. LC/MS (ESI) (m/z): 750 (M+H).

### Step 9: ((S)-N-((R)-1-(1H-pyrrolo[3,2-c]pyridin-2-yl)ethyl)-2-((phenoxathiine-3-carbonyl)glycyl)-2-azaspiro[4.5]decane-3-carboxamide (Compound 234)

To a solution of (S)-2-((phenoxathiine-3-carbonyl)glycyl)-N-((R)-1-(1-(phenylsulfonyl)-1H-pyrrolo[3,2-c]pyridin-2-yl)ethyl)-2-azaspiro[4.5]decane-3-carboxamide (23 mg, 0.03 mol) in MeOH (1 mL) and water (0.25 mL) was added LiOH·H₂O (3 mg, 0.06 mol) and the mixture was stirred at room temperature for 2 hours. The mixture was concentrated under reduced pressure to dryness. The residue was purified by prep-HPLC to give Compound 234 (2.8 mg, yield 15.4%) as a white solid. ¹H NMR (400 MHz, CD₃OD): δ 8.64 (s, 1H), 7.89 (d, J=5.6 Hz, 1H), 7.64-7.55 (m, 2H), 7.28 (d, J=8.0 Hz, 1H), 7.22-7.15 (m, 2H), 7.10 (d, J=6.0 Hz, 2H), 7.04 (d, J=7.7 Hz, 1H), 6.51 (s, 1H), 5.31 (d, J=6.7 Hz, 1H), 4.46-4.36 (m, 2H), 4.11 (d, J=16.6 Hz, 1H), 3.72 (d, J=9.9 Hz, 1H), 3.40 (d, J=10.0 Hz, 1H), 2.27-2.20 (m, 1H), 1.80-1.74 (m, 1H), 1.60 (d, J=6.8 Hz, 3H), 1.55-1.42 (m, 8H), 1.36-1.28 (m, 2H). LC/MS (ESI) m/z: 610 (M+H). RT (Method A): 1.80 min.

### Scheme 68. Synthesis of (2S,4R)-N-((4-amino-2-oxo-2H-benzo[e][1,3]oxazin-7-yl)methyl)-4-(difluoromethoxy)-1-((phenoxathiine-3-carbonyl)glycyl)pyrrolidine-2-carboxamide (Compound 248)

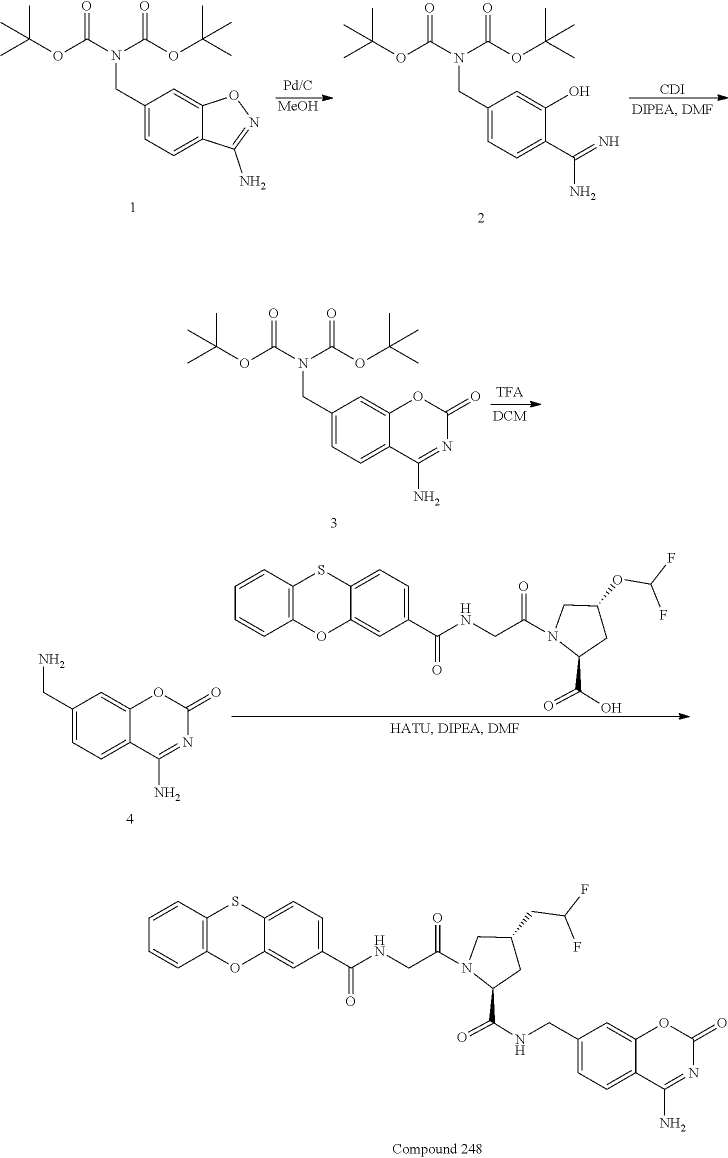

### Step 1: Tert-butyl (tert-butoxycarbonyl)(4-carbamimidoyl-3-hydroxybenzyl) carbamate (2)

To a solution of tert-butyl ((3-aminobenzo[d]isoxazol-6-yl)methyl)(tert-butoxycarbonyl)carbamate (50 mg. 0.14 mmol) in MeOH (2 mL) was added Pd/C (5 mg, 10% wt.), the mixture was degassed under N₂ atmosphere for three times and stirred under a H₂ balloon at room temperature for 3 hours. The mixture was filtered, and the filtrate was concentrated to dryness to give the title compound (50 mg, yield 99.4%) as a yellow solid, which was used directly in the next step without further purification. LC/MS (ESI) m/z: 366 (M+H)⁺.

### Step 2: Tert-butyl ((4-amino-2-oxo-2H-benzo[e][1,3]oxazin-7-yl)methyl)(tert-butoxycarbonyl)carbamate (3)

To a solution of tert-butyl (tert-butoxycarbonyl)(4-carbamimidoyl-3-hydroxybenzyl) carbamate (50 mg, 0.14 mmol) in DMF (1 mL) was added DIPEA (53 mg, 0.41 mmol) and CDI (44 mg, 0.27 mmol) and the mixture was stirred at room temperature for overnight. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=10:1) to give the title compound (37 mg, yield 69.0%) as a white solid. LC/MS (ESI) m/z: 392(M+H)⁺.

### Step 3: 4-Amino-7-(aminomethyl)-2H-benzo[e][1,3]oxazin-2-one (4)

To a solution of tert-butyl ((4-amino-2-oxo-2H-benzo[e][1,3]oxazin-7-yl)methyl)(tert-butoxycarbonyl)carbamate (35 mg, 0.089 mmol) in DCM (0.9 mL) was added TFA (0.3 mL) at 0° C. and the mixture was stirred under N₂ atmosphere at room temperature for 2 hours. The reaction mixture was concentrated to dryness under reduced pressure to give the title compound (16 mg, yield 93.5%) as a yellow solid, which was used directly in the next step without further purification. LC/MS (ESI) m/z: 192 (M+H)⁺.

### Step 4: (2S,4R)-N-((4-amino-2-oxo-2H-benzo[e][1,3]oxazin-7-yl)methyl)-4-(difluoromethoxy)-1-((phenoxathiine-3-carbonyl)glycyl)pyrrolidine-2-carboxamide (Compound 248)

To a mixture of (2S,4R)-4-(difluoromethoxy)-1-((phenoxathiine-3-carbonyl)glycyl) pyrrolidine-2-carboxylic acid (40 mg, 0.086 mol) and 4-amino-7-(aminomethyl)-2H-benzo[e][1,3]oxazin-2-one (16 mg, 0.083 mmol) in DMF (2 mL) was added DIPEA (55 mg, 0.42 mmol) and HATU (42 mg, 0.11 mol) under N₂ atmosphere and the reaction mixture was stirred at room temperature for 1 hour. The mixture was concentrated under reduced pressure to dryness. The residue was purified by prep-HPLC to give Compound 248 (9 mg, yield 16.9%) as a white solid. ¹H NMR (400 MHz, CD₃OD): δ 7.89 (d, J=8.2 Hz, 1H), 7.54-7.51 (m, 1H), 7.41 (d, J=1.8 Hz, 1H), 7.28 (d, J=8.3 Hz, 1H), 7.25 (s, 1H), 7.22 (d, J=8.1 Hz, 1H), 7.21-7.15 (m, 2H), 7.09 (dd, J=7.5, 1.3 Hz, 1H), 7.04 (dd, J=8.1, 1.1 Hz, 1H), 6.75-6.37 (m, 1H), 5.01 (s, 1H), 4.61 (d, J=7.8 Hz, 1H), 4.52 (d, J=4.0 Hz, 2H), 4.18 (d, J=9.7 Hz, 2H), 3.99 (dd, J=11.4, 4.5 Hz, 1H), 3.88 (d, J=12.2 Hz, 1H), 2.53-2.46 (m, 1H), 2.28-2.21 (m, 1H). LC/MS (ESI) m/z: 638 (M+H)⁺. RT (Method A): 1.74 min.

### Scheme 69. Synthesis of (S)-7-((3-(4-fluorophenoxy)benzoyl)glycyl)-N-(thieno[3,2-c]pyridin-2-ylmethyl)-1,4-dioxa-7-azaspiro[4.4]nonane-8-carboxamide (Compound 255)

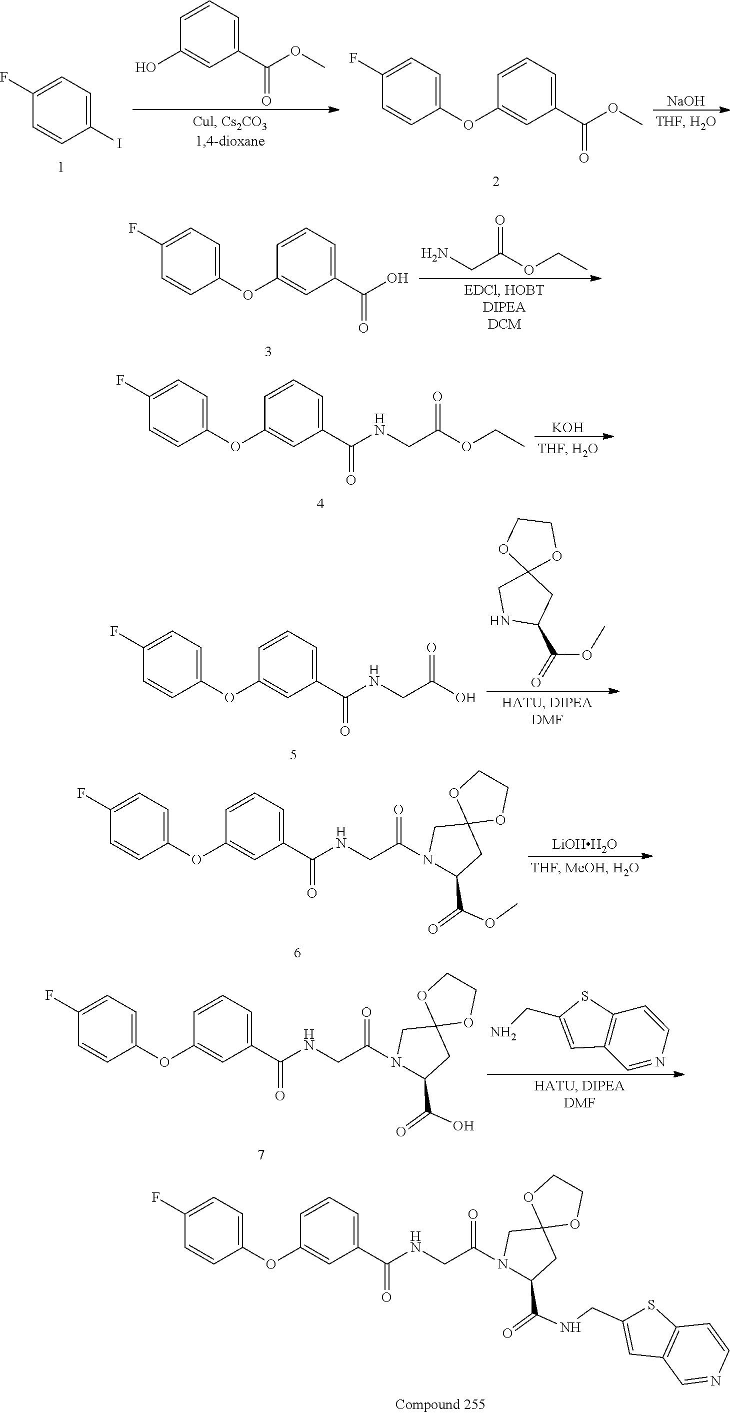

### Step 1: Methyl 3-(4-fluorophenoxy)benzoate (2)

To a mixture of 1-fluoro-4-iodobenzene (2.0 g, 13.3 mmol) and methyl 3-hydroxybenzoate (5.8 g, 26.3 mmol) in 1,4-dioxane (20 mL) was added CsCO₃ (6.42 g, 19.7 mmol) followed by CuI (1.4 g, 7.25 mmol) under N₂ atmosphere, and the mixture was stirred at 100° C. for 12 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel. 0-30% EtOAc in PE) to give the title compound (450 mg, yield 14.4%) as a white solid. LC/MS (ESI) m/z: 247 (M+H)⁺.

### Step 2: 3-(4-Fluorophenoxy)benzoic acid (3)

To a solution of methyl 3-(4-fluorophenoxy)benzoate (450 mg, 1.83 mmol) in THF (5 mL) was added NaOH (219 mg, 5.48 mmol) and the reaction mixture was stirred at room temperature for 2 hours. The mixture was acidified with IN aq. HCl to pH˜3 and extracted with EtOAc twice. The combined organic layers were dried over anhydrous Na₂SO₄, filtered, and concentrated under reduced pressure to give the title compound (300 mg, yield 70.8%) as a white solid. LC/MS (ESI) m/z: 233 (M+H)⁺.

### Step 3: Ethyl (3-(4-fluorophenoxy)benzoyl)glycinate (4)

To a mixture of 3-(4-fluorophenoxy)benzoic acid (300 mg, 1.29 mmol) and ethyl glycinate (270 mg, 1.93 mmol) in DCM (10 mL) was added DIPEA (334 mg, 0.23 mmol) followed by H OBT (262 mg, 1.93 mmol) under N₂ atmosphere, and the reaction mixture was stirred at room temperature for 2 hours. The mixture was diluted with DCM, dried over anhydrous Na₂SO₄, filtered, and concentrated under reduced pressure to dryness. The residue was purified by flash chromatography (silica gel, 0-100% EtOAc in PE) to give the title compound (280 mg, yield 58.0%) as a white solid. LC/MS (ESI) m/z: 318 (M+H)⁺.

### Step 4: (3-(4-Fluorophenoxy)benzoyl)glycine (5)

To a solution of ethyl (3-(4-fluorophenoxy)benzoyl)glycinate (280 mg, 0.88 mmol) in THF (3 mL) and H₂O (1.5 mL) was added KOH (100 mg, 1.76 mmol) and the mixture was stirred at 70° C. for 2 hours. The mixture was acidified with 1N aq. HCl to pH˜3 and extracted with EtOAc twice. The combined organic layers were dried over anhydrous Na₂SO₄, filtered, and concentrated under reduced pressure to give the title compound (210 mg, yield 76.9%) as a white solid. LC/MS (ESI) m/z: 290 (M+H)⁺.

### Step 5: Methyl (S)-7-((3-(4-fluorophenoxy)benzoyl)glycyl)-1,4-dioxa-7-azaspiro[4.4]nonane-8-carboxylate (6)

To a mixture of (3-(4-fluorophenoxy)benzoyl)glycine (100 mg, 0.35 mmol) and methyl (S)-1,4-dioxa-7-azaspiro[4.4]nonane-8-carboxylate (96.7 mg, 0.52 mmol) in DMF (2 mL) was added DIPEA (133 mg, 1.03 mmol) followed by HA TU (196 mg, 0.52 mmol) under N₂ atmosphere, and the reaction mixture was stirred at room temperature for 2 hours. The mixture was diluted with EtOAc, dried over anhydrous Na₂SO₄, filtered, and concentrated under reduced pressure to dryness. The residue was purified by flash chromatography (silica gel. 0-100% EtOAc in PE) to give the title compound (40 mg, yield 25.3%) as a white solid. LC/MS (ESI) m/z: 459 (M+H)⁺.

### Step 6: (S)-7-((3-(4-fluorophenoxy)benzoyl)glycyl)-1,4-dioxa-7-azaspiro[4.4]nonane-8-carboxylic acid (7)

To a solution of methyl (S)-7-((3-(4-fluorophenoxy)benzoyl)glycyl)-1,4-dioxa-7-azaspiro[4.4]nonane-8-carboxylate (40 mg, 0.09 mmol) in THF/MeOH/H₂O (2 mL, 4/1/1) was added LiOH (7 mg, 0.18 mmol) and the mixture was stirred at room temperature for 2 hours. The mixture was acidified with 1N aq. HCl to pH˜3 and extracted with EtOAc twice. The combined organic layers were dried over anhydrous Na₂SO₄, filtered, and concentrated under reduced pressure to give the title compound (27 mg, yield 69.9%) as a white solid. LC/MS (ESI) m/z: 445 (M+H)⁺.

### Step 7: (S)-7-((3-(4-fluorophenoxy)benzoyl)glycyl)-N-(thieno[3,2-c]pyridin-2-ylmethyl)-1,4-dioxa-7-azaspiro[4.4]nonane-8-carboxamide (Compound 255)

To a mixture of (S)-7-((3-(4-fluorophenoxy)benzoyl)glycyl)-1,4-dioxa-7-azaspiro[4.4]nonane-8-carboxylic acid (27 mg, 0.06 mmol) and thieno[3,2-c]pyridin-2-ylmethanamine (15 mg, 0.09 mmol) in DMF (2 mL) was added DIPEA (24 mg, 0.18 mmol) followed by HATU (35 mg, 0.09 mmol) under N₂ atmosphere and the reaction mixture was stirred at room temperature for 2 hours. The mixture was diluted with EtOAc, dried over anhydrous Na₂SO₄, filtered, and concentrated under reduced pressure to dryness. The residue was purified by prep-HPLC to give Compound 255 (4 mg, yield 11.4%) as a white solid. ¹H NMR (400 MHz, CD₃OD): δ 8.87 (s, 1H), 8.27 (d, J=5.6 Hz, 1H), 7.88 (d, J=5.7 Hz, 1H), 7.55 (d, J=8.3 Hz, 1H), 7.43-7.38 (m, 3H), 7.10 (t, J=8.6 Hz, 3H), 7.04-7.00 (m, 2H), 4.81-4.78 (m, 1H), 4.74 (d, J=5.9 Hz, 1H), 4.61 (dd, J=9.1, 5.3 Hz, 1H), 4.18 (d, J=16.6 Hz, 1H), 4.10 (d, J=16.7 Hz, 1H), 3.98 (d, J=1.5 Hz, 4H), 3.79 (s, 2H), 2.48-2.40 (m, 1H), 2.29-2.19 (m, 1H). LC/MS (ESI) m/z: 591 (M+H)⁺. RT (Method A): 1.31 min.

### Scheme 70. Synthesis of (2S,4R)-N-((1H-pyrrolo[3,2-c]pyridin-2-yl)methyl)-4-(difluoromethoxy)-1-((3-(2,4-difluorophenoxy)benzoyl)glycyl)pyrrolidine-2-carboxamide (Compound 259)

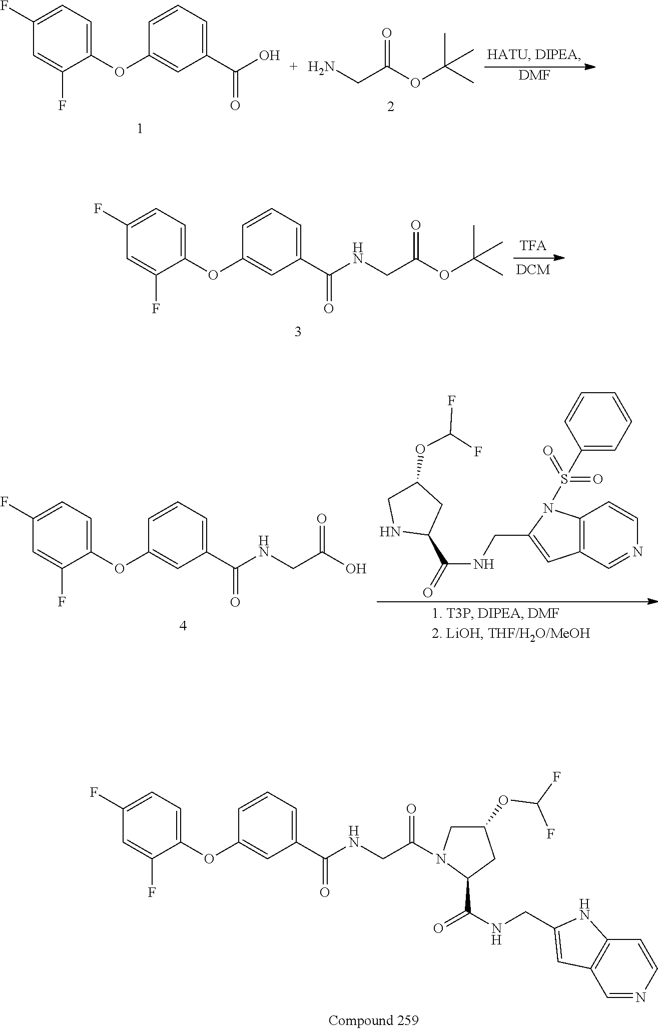

### Step 1: Tert-butyl (3-(2,4-difluorophenoxy)benzoyl)glycinate (3)

To a solution of 3-(2,4-difluorophenoxy)benzoic acid (90.00 mg, 0.3597 mmol, 1 equiv.) in DMF (2 mL) was added tert-butyl 2-aminoacetate hydrochloride (72.40 mg, 0.4319 mmol, 1.2 equiv.), HATU (205.0 mg, 0.5391 mmol, 1.3 equiv.) and DIPEA (0.188 mL, 1.08 mmol, 3 equiv.). The reaction mixture was stirred at room temperature for 1 h. Water (5 mL) was added stirred for 10 minutes. The product crashed out was collected by filtration and dried to give tert-butyl 2-[[3-(2,4-difluorophenoxy)benzoyl]amino]acetate (104.0 mg, 0.2862 mmol, 79.56% yield). LC/MS (ESI) m/z: 364 (M+H)⁺.

### Step 2: (3-(2,4-difluorophenoxy)benzoyl)glycine (4)

TFA (0.5 mL) was added to a solution of tert-butyl 2-[[3-(2,4-difluorophenoxy)benzoyl]amino]acetate (100.0 mg, 0.2752 mmol, 1.0 equiv.) in CH₂Cl₂ (1 mL) at ice-bath temperature. The reaction mixture was stirred at room temperature for 4 hours and then concentrated to dryness to give 2-[[3-(2,4-difluorophenoxy)benzoyl]amino]acetic acid (84.00 mg, 0.2734 mmol, 99.37% yield) as a clear oil which was used in the next step without further purification. LC/MS (ESI) m/z:308 (M+H)⁺.

### Steps 3 and 4: (2S,4R)-N-((JH-pyrrolo[3,2-c]pyridin-2-yl)methyl)-4-(difluoromethoxy)-1-((3-(2,4-difluorophenoxy)benzoyl)glycyl)pyrrolidine-2-carboxamide (Compound 259)

To a solution of 2-[[3-(2,4-difluorophenoxy)benzoyl]amino]acetic acid (17.35 mg, 0.05648 mmol, 1.0 equiv.) in DMF (2 mL) was added (2S,4R)-N-[[1-(benzenesulfonyl)pyrrolo[3,2-c]pyridin-2-yl]methyl]-4-(difluoromethoxy)pyrrolidine-2-carboxamide;hydrochloride (25.00 mg, 0.05135 mmol, 1.0 equiv.), T3P, (50 mass %) in ETHYL ACETATE (46.10 μL, 0.0770 mmol, 1.5 equiv.), and DIPEA (26.90 μL, 0.154 mmol, 3.0 equiv.). The reaction mixture was stirred at room temperature for 30 minutes and then diluted with water. The filtrate was decanted, and the residue was washed with water, dried over Na₂SO₄, and concentrated to give a brown oil which was used in the next reaction without further purification. To the brown oil obtained above in THF (1.5 mL), MeOH (0.5 mL) and Water (0.5 mL) was added LiOH (2.459 mg, 0.1027 mmol, 2 equiv.), the reaction mixture was stirred at room temperature for 6 hours and then concentrated to remove THF/MeOH. The pH was adjusted to 1 using 4M HCl, concentrated and purified by prep HPLC to give Compound 259 (3.450 mg, 0.005755 mmol, 100 mass %, 11.21% yield). LC/MS (ESI) m/z: 600 (M+H)⁺. ¹H NMR (400 MHz, DMSO-d6): δ 11.25 (s, 1H), 8.94 (d, J=5.9 Hz, 1H), 8.89-8.75 (m, 1H), 8.69 (d, J=14.2 Hz, 1H), 8.32 (s, 1H), 8.03 (d, J=5.6 Hz, 1H), 7.65 (t, J=8.3 Hz, 1H), 7.57-7.46 (m, 3H), 7.42-7.26 (m, 2H), 7.23-7.03 (m, 4H), 6.82 (s, 1H), 6.38 (s, 1H), 4.97 (s, 1H), 4.50 (dd, J=9.9, 5.3 Hz, 1H), 4.19 (dd, J=16.8, 5.8 Hz, 1H), 4.01 (dd, J=16.7, 5.7 Hz, 1H), 3.87 (dd, J=11.6, 4.6 Hz, 1H), 3.82-3.64 (m, 1H), 2.33 (td, J=10.5, 4.8 Hz, 2H), 2.20-7.8.1(m, 1H) RT (Method A): 1.38 min.

Compound 153 was prepared based on Scheme 70:

| Cmpd # | Reactant A | Reactant B | Reactant C | Characterization Data |
|---|---|---|---|---|
| 153^{a} | 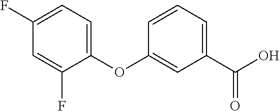 [SMILES: O=C(O)c1cccc(Oc2ccc(F)cc2F)c1] | 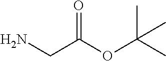 | 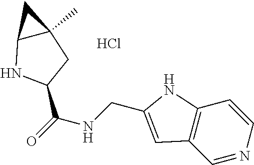 | ¹H NMR (400 MHz, DMSO-d6): δ 11.33 (s, 1H), 8.70 (dd, J = 13.5, 7.8 Hz, 1H), 8.38 (t, J = 5.8 Hz, 1H), 8.20-7.76 (m, 1H), 7.58 (d, J = 7.6 Hz, 1H), 7.47-7.35 (m, 2H), 7.24-7.09 (m, 4H), 7.03 (dt, J = 9.1, 4.6 Hz, 2H), 6.34 (s, 1H), 4.63 (dd, J = 11.3, 3.3 Hz, 1H), 4.47-4.13 (m, 1H), 3.98 (dd, J = 16.5, 5.7 Hz, 1H), 3.36 (dd, J - 5.9, 2.4 Hz, 1H), 2.34-2.12 (m, 1H), 1.98 (dd, J = 13.2, 3.3 Hz, 1H), 1.54-1.32 (m, 4H), 1.32-1.03 (m, 5H), 0.86 (q, J = 7.6 Hz, 2H), 0.63 (t, J = 5.5 Hz, 1H). LC/MS (ESI) m/z: 542 (M + H)⁺. RT (Method A): 1.54 min. |

^{a}Deprotection was not necessary in Step 3.

### Scheme 71. Synthesis of (2S,4R)-N-((1H-pyrazolo[4,3-b]pyridin-6-yl)methyl)-4-(difluoromethoxy)-1-((phenoxathiine-3-carbonyl)glycyl)pyrrolidine-2-carboxamide (Compound 262)

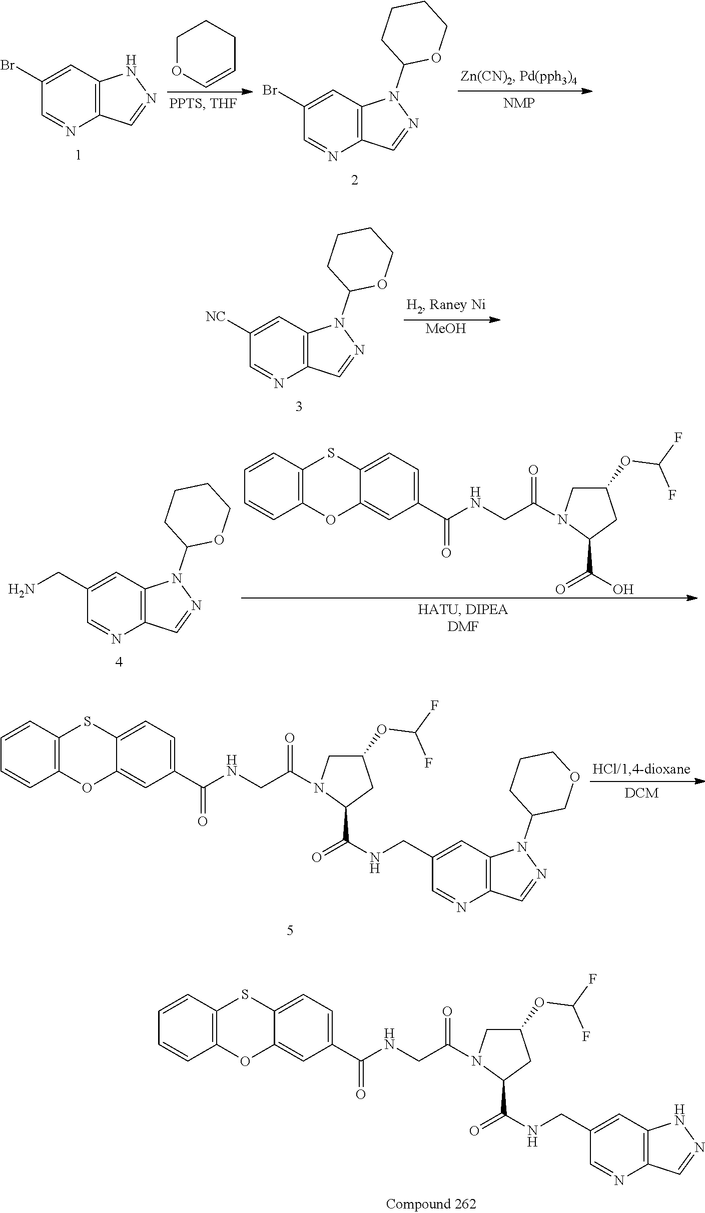

### Step 1: 6-Bromo-1-(tetrahydro-2H-pyran-2-yl)-1H-pyrazolo[4,3-b]pyridine (2)

To a mixture of 6-bromo-1H-pyrazolo[4,3-b]pyridine (400 mg, 2.02 mmol) and 3,4-dihydro-2H-pyran (255 mg, 3.03 mmol) in THF (5 mL) was added PPTS (507 mg, 2.02 mmol) and the reaction mixture was stirred at 50° C. overnight. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-25% EtOAc in PE) to give the title compound (362 mg, yield 63.8%) as a colorless oil. LC/MS (EST) m/z: 282 (M1+H)⁺.

### Step 2: 1-(Tetrahydro-2H-pyran-2-yl)-1H-pyrazolo[4,3-b]pyridine-6-carbonitrile (3)

To a mixture of 6-bromo-1-(tetrahydro-2H-pyran-2-yl)-1H-pyrazolo[4,3-b]pyridine (362 mg, 1.29 mmol) and Zn(CN)₂ (303 mg, 2.58 mmol) in NMP (5 mL) was added Pd(PPh₃)₄ (149 mg, 0.129 mmol) and the reaction mixture was stirred at 100° C. overnight. The mixture was diluted with EtOAc, washed with brine, dried over anhydrous Na₂SO₄, filtered, and concentrated under reduced pressure to dryness. The residue was purified by flash chromatography (silica gel, 0-28% EtOAc in PE) to give the title compound (123 mg, yield 53.9%) as a yellow oil. LC/MS (ESI) m/z: 229 (M+H)⁺.

### Step 3: (1-(tetrahydro-2H-pyran-2-yl)-1H-pyrazolo[4,3-b]pyridin-6-yl)methanamine (4)

To a solution of 1-(tetrahydro-2H-pyran-2-yl)-1H-pyrazolo[4,3-b]pyridine-6-carbonitrile (120 mg, 0.53 mmol) in MeOH (2 mL) was added Raney Ni (25 mg, wet). The mixture was degassed under N₂ atmosphere for three times and stirred under a H₂ balloon at 25° C. overnight. The mixture was filtered, and the filtrate was concentrated to dryness to give the title compound (100 mg, yield 81.9%) as a yellow oil, which was used in next reaction without purification. LC/MS (ESI) m/z: 233 (M+H)⁺.

### Step 4: (2S,4R)-4-(difluoromethoxy)-1-((phenoxathiine-3-carbonyl)glycyl)-N-((1-(tetrahydro-2H-pyran-2-yl)-1H-pyrazolo[4,3-b]pyridin-6-yl)methyl)pyrrolidine-2-carboxamide (5)

To a mixture of (2S,4R)-4-(difluoromethoxy)-1-((phenoxathiine-3-carbonyl)glycyl) pyrrolidine-2-carboxylic acid (50 mg, 0.11 mmol) and (1-(tetrahydro-2H-pyran-2-yl)-1H-pyrazolo[4,3-b]pyridin-6-yl)methanamine (50 mg, 0.22 mmol) in DMF (2 mL) was added DIPEA (84 mg, 0.65 mmol) and H ATU (49 mg, 0.13 mmol) under N₂ atmosphere and the reaction mixture was stirred at room temperature for 2 hours. The mixture was diluted with EtOAc, washed with saturated aq. NH₄Cl solution, dried over anhydrous Na₂SO₄, filtered, and concentrated under reduced pressure to dryness. The residue was purified by flash chromatography (silica gel, 0-5% MeOH in DCM) to give the title compound (50 mg, yield 68.5%) as a white solid. LC/MS (ESI) m/z: 679 (M+H)⁺.

### Step 5: (2S,4R)-N-((1H-pyrazolo[4,3-b]pyridin-6-yl)methyl)-4-(difluoromethoxy)-1-((phenoxathiine-3-carbonyl)glycyl)pyrrolidine-2-carboxamide (Compound 262)

To a solution of (2S,4R)-4-(difluoromethoxy)-1-((phenoxathiine-3-carbonyl)glycyl)-N-((1-(tetrahydro-2H-pyran-2-yl)-1H-pyrazolo[4,3-b]pyridin-6-yl)methyl)pyrrolidine-2-carboxamide (30 mg, 0.04 mmol) in DCM (1 mL) was added HCl/1,4-dioxane (3 mL, 4 M) and the mixture was stirred at room temperature for 2 hours. The reaction mixture was concentrated to dryness. The residue was purified by prep-HPLC to give Compound 262 (4.5 mg, yield 17.1%) as a white solid. ¹H NMR (400 MHz, CD₃OD): δ 8.42 (d, J=1.5 Hz, 1H), 8.07 (s, 1H), 7.93 (s, 1H), 7.42-7.39 (m. 1H), 7.34 (d, J=1.7 Hz, 1H), 7.14 (s, 1H), 7.12-7.06 (m, 2H), 6.99 (m, 2H), 4.97 (s, 1H), 4.57 (m, 4H), 4.23-4.09 (m, 2H), 3.96-3.90 (m, 1H), 3.82 (d, J=12.2 Hz, 1H), 2.50-2.41 (m, 1H), 2.26-2.15 (m, 1H). LC/MS (ESI) m/z: 595 (M+H)⁺. RT (Method A): 1.79 min.

### Scheme 72. Synthesis of (2S,4R)-4-(difluoromethoxy)-N-((2,3-dihydro-1H-pyrrolo [3,2-b]pyridin-5-yl)methyl)-1-((phenoxathiine-3-carbonyl)glycyl)pyrrolidine-2-carboxamide (Compound 264)

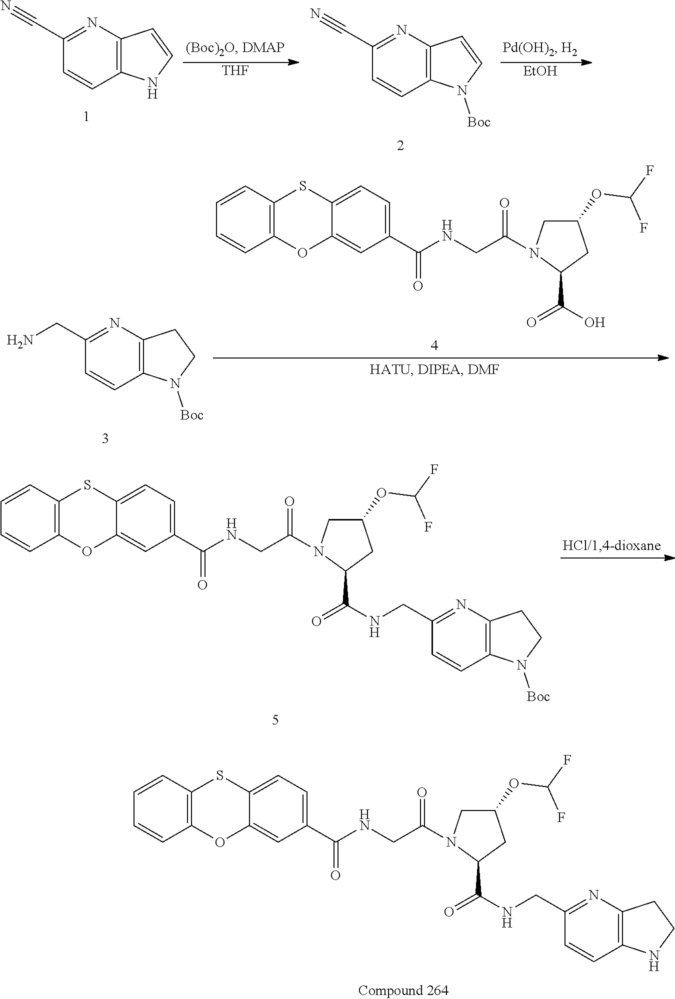

### Step 1: Tert-butyl 5-cyano-1H-pyrrolo[3,2-b]pyridine-1-carboxylate (2)

To a solution of 1H-pyrrolo[3,2-b]pyridine-5-carbonitrile (42 mg, 0.29 mmol) in THF (2.0 mL) was added (Boc)₂O (126 mg, 0.58 mmol) and DMAP (4 mg, 0.03 mmol) at 0° C. and the mixture was stirred at room temperature for 16 hours. The mixture was diluted with water and extracted with EtOAc twice. The combined organic layers were washed with brine, dried over anhydrous Na₂SO₄, filtered, and concentrated to dryness. The residue was purified by flash chromatography (silica gel, 0-50% EtOAc in PE) to give the title compound (70 mg, yield 99.2%) as a white solid. LC/MS (ESI) n/z: 244 (M+H)⁺.

### Step 2: (Tert-butyl 5-(aminomethyl)-2,3-dihydro-1H-pyrrolo[3,2-b]pyridine-1-carboxylate (3)

To a mixture of tert-butyl 5-cyano-1H-pyrrolo[3,2-b]pyridine-1-carboxylate (68 mg, 0.28 mmol) in EtOH (2 mL) was added Pd(OH)₂/C (10 mg, 10% wt.), the mixture was degassed under N₂ atmosphere for three times and stirred under a H₂ balloon at room temperature for 2 hours. The reaction mixture was concentrated under reduced pressure to dryness to give the title compound (60 mg, yield 86.0%) as a white solid. LC/MS (ESI) (m/z): 250 (M+H)⁻.

### Step 3: Tert-butyl 5-(((2S,4R)-4-(difluoromethoxy)-1-((phenoxathiine-3-carbonyl) glycyl)pyrrolidine-2-carboxamido)methyl)-2,3-dihydro-1H-pyrrolo[3,2-b]pyridine-1-carboxylate (4)

To a mixture of (2S,4R)-4-(difluoromethoxy)-1-((phenoxathiine-3-carbonyl)glycyl) pyrrolidine-2-carboxylic acid (46 mg, 0.10 mmol) and (tert-butyl 5-(aminomethyl)-2,3-dihydro-1H-pyrrolo[3,2-b]pyridine-1-carboxylate (60 mg, 0.24 mmol) in DMF (0.5 mL) was added DIPEA (40 mg, 0.30 mmol) and HATU (46 mg, 0.12 mmol) under N₂ atmosphere and the reaction mixture was stirred at room temperature for 1 hour. The mixture was diluted with EtOAc, washed with brine, dried over anhydrous Na₂SO₄, filtered, and concentrated under reduced pressure to dryness. The residue was purified by prep-TLC (DCM:MeOH=12:1) to give the title compound (40 mg, yield 58.0%) as a yellow solid. LC/MS (ESI) m/z: 696 (M+H)⁺.

### Step 4: (2S,4R)-4-(difluoromethoxy)-N-((2,3-dihydro-1H-pyrrolo[3,2-b]pyridin-5-yl)methyl)-1-((phenoxathiine-3-carbonyl)glycyl)pyrrolidine-2-carboxamide (Compound 264)

A solution of tert-butyl 5-(((2S,4R)-4-(difluoromethoxy)-1-((phenoxathiine-3-carbonyl) glycyl)pyrrolidine-2-carboxamido)methyl)-2,3-dihydro-1H-pyrrolo[3,2-b]pyridine-1-carboxylate (20 mg, 0.029 mmol) in HCl/1,4-dioxane (0.5 mL, 4M) was stirred under N₂ atmosphere at room temperature for 1 hour. The reaction mixture was concentrated to dryness. The residue was purified by prep-HPLC to give Compound 264 (3.6 mg, yield 21.1%) as a white solid. ¹H NMR (400 MHz, CD₃OD): δ 7.54 (d, J=8.1, 1.8 Hz, 1H), 7.49 (d, J=1.8 Hz, 1H), 7.24-7.15 (m, 3H), 7.07 (d, J=14.7, 7.7 Hz, 2H), 6.98 (d, J=8.1 Hz, 1H), 6.83 (d, J=13.3, 8.1 Hz, 1H), 6.51 (t, J=74.5 Hz, 1H), 4.37 (s, 2H), 4.20 (d, 0.1=25.9 Hz, 2H), 4.00-3.95 (m, 1H), 3.85 (d, 0.1=11.0 Hz, 1H), 3.55 (t, J=8.6 Hz, 2H), 3.03 (t, J=8.6 Hz, 2H), 2.47 (s, 1H), 2.31-2.23 (m, 1H), 2.22-2.16 (m, 1H), 2.04 (s, 1H). LC/MS (ESI) m/z: 596 (M+H)⁺. RT (Method A): 1.59 min.

Compounds 265 and 374 were prepared based on Scheme 72:

| Cmpd # | Reactant A | Reactant B | Characterization Data |
|---|---|---|---|
| 265 | 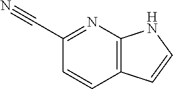 [SMILES: N#Cc1ccc2cc[nH]c2n1] | 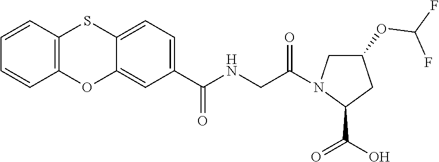 [SMILES: O=C(NCC(=O)N1C[C@H](OC(F)F)C[C@H]1C(=O)O)c1ccc2c(c1)Oc1ccccc1S2] | ¹H NMR (400 MHz, CD₃OD): δ 7.54-7.51 (m, 1H), 7.46 (d, J = 1.8 Hz, 1H), 7.22 (s, 1H), 7.20 (s, 1H), 7.15 (d, J = 1.6 Hz, 1H), 7.14 (d, J = 1.5 Hz, 1H), 7.09- 7.07 (m, 1H), 7.05 (s, 1H), 6.51 (d, J = 5.9 Hz, 1H), 6.48-6.33 (m, 1H), 5.03- 5.00 (m, 1H), 4.58 (s, 2H), 4.27 (d, J = 2.3 Hz, 2H), 4.19 (d, J = 18.8 Hz, 2H), 4.00-3.96 (m, 1H), 3.53 (d, J = 8.4 Hz, 2H), 2.97-2.93 (m, 2H), 2.50-2.45 (m, 1H), 2.31-2.25 (m, 1H). LC/MS (ESI) m/z: 596 (M + H)⁺. RT (Method A): 1.54 min. |
| 374^{a,b} | 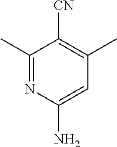 | 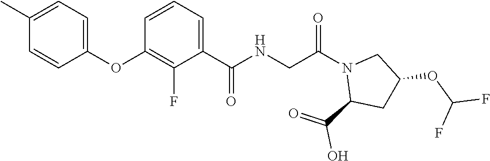 [SMILES: Cc1ccc(Oc2cccc(C(=O)NCC(=O)N3C[C@H](OC(F)F)C[C@H]3C(=O)O)c2F)cc1] | ¹H NMR (400 MHz, CD₃OD): δ 7.55 (s, 1H), 7.22 (d, J = 7.7 Hz, 1H), 7.20- 7.16 (m, 3H), 6.91-6.88 (m, 2H), 6.70-6.24 (m, 2H), 5.00 (s, 1H), 4.59 (s, 1H), 4.52 (t, J = 7.8 Hz, 1H), 4.33 (d, J = 2.8 Hz, 2H), 4.20-4.13 (m, 1H), 3.93-3.90 (m, 1H), 3.83 (s, 1H), 3.49 (s, 1H), 3.14 (s, 1H), 2.40 (s, 1H), 2.37 (d, J = 7.1 Hz, 3H), 2.33 (s, 3H), 2.25 (s, 3H). LC/MS (ESI) m/z: 600 (M + H)⁺. RT (Method A): 1.55 min. |

^{a}Step 1 was performed with an 5-fold excess of TEA relative to 6-amino-2,4-dimethylnicotinonitrile and 1,4-dioxane as the solvent.
^{b}Step 2 was performed using Raney Ni as the hydrogenation catalyst.

### Scheme 73. Synthesis of (2S,4R)-4-(difluoromethoxy)-1-((phenoxathiine-3-carbonyl) glycyl)-N-(pyrrolo[1,2-a]pyrazin-7-ylmethyl)pyrrolidine-2-carboxamide (Compound 266)

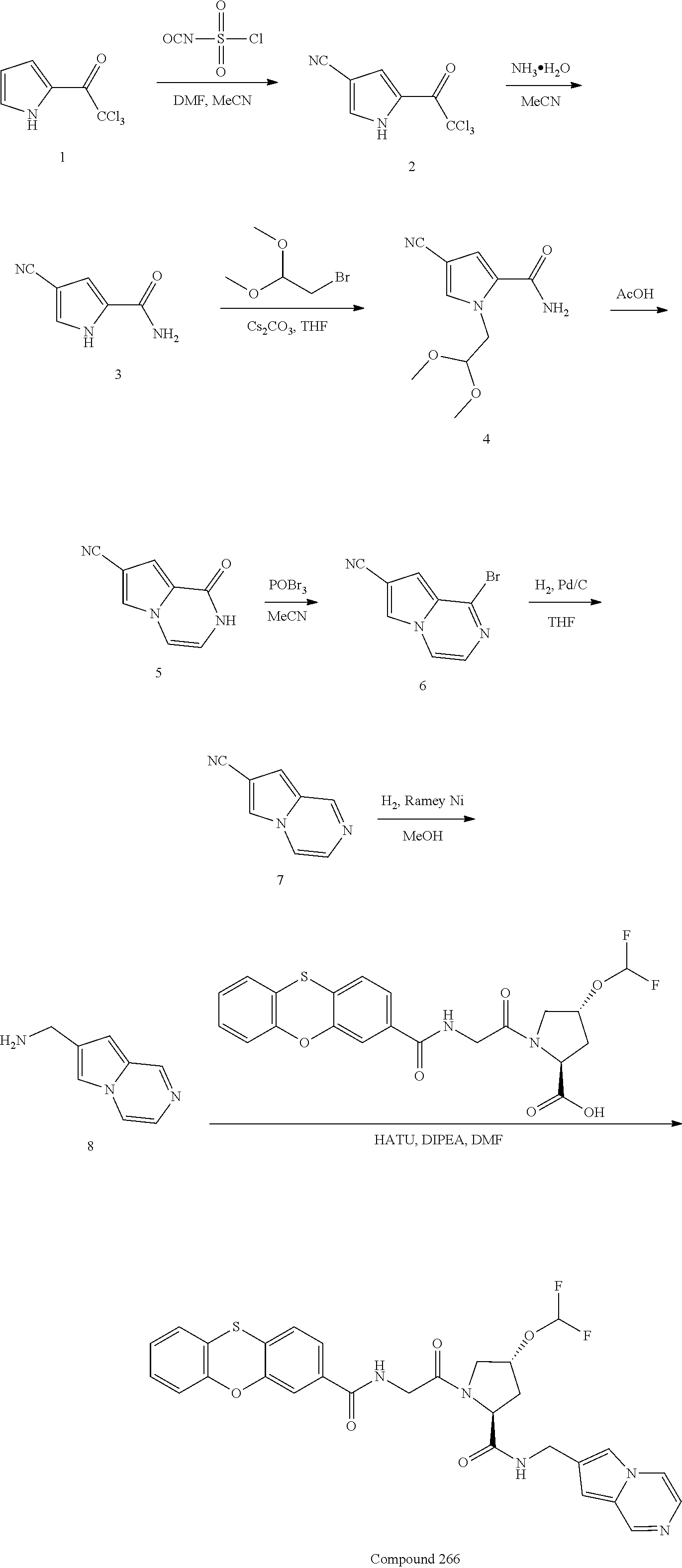

### Step 1: 5-(2, 2,2-Trichloroacetyl)-1H-pyrrole-3-carbonitrile (2)

To a solution of 2,2,2-trichloro-1-(1H-pyrrol-2-yl)ethan-1-one (4.5 g, 21.0 mmol) in MeCN (50 mL) was added chlorosulfonyl isocyanate (4.0 mL, 45 mmol) at 0° C. and the mixture was stirred at room temperature for 3 hours. DMF (15 mL) was added to the reaction mixture and the resulting mixture was stirred at room temperature overnight. The reaction mixture was diluted with water and extracted with EtOAc twice. The combined organic layers were washed with brine, dried over anhydrous Na₂SO₄, filtered, and concentrated to dryness. The residue was purified by flash chromatography (silica gel, 0-50% EA in PE) to give the title compound (4.2 g, yield 83.5%) as a yellow solid. ¹H NMR (400 MHz, CDCl₃) δ 11.08 (s, 1H), 7.53 (s, 1H), 7.51 (s, 1H).

### Step 2: 4-Cyano-1H-pyrrole-2-carboxamide (3)

To a solution of 5-(2,2,2-trichloroacetyl)-1H-pyrrole-3-carbonitrile (2.50 g, 10.5 mmol) in MeCN (20 mL) was added NH₃·H₂O (7.0 mL, 25 wt. % in H₂O) at 0° C. and the mixture was stirred at room temperature for 2 hours. The precipitate was filtered, washed with EtOAc, dried under vacuum to give the title compound (1.20 g, yield 84.4%) as a white solid, which was used directly in the next step without further purification. ¹H NMR (400 MHz, DMSO-d6) δ 12.51 (s, 1H), 7.86 (s, 1H), 7.72 (s, 1H), 7.35 (s, 1H), 7.21 (s, 1H). LC/MS (ESI) m/z: 136 (M+H)⁺.

### Step 3: 4-Cyano-1-(2,2-dimethoxyethyl)-1H-pyrrole-2-carboxamide (4)

To a mixture of 4-cyano-1H-pyrrole-2-carboxamide (1.20 g, 8.88 mmol) in DMF (15 mL) was added Cs₂CO₃ (5.79 g, 17.8 mmol) followed by 2-bromo-1, l-dimethoxyethane (2.25 g, 13.3 mmol) under N₂ atmosphere and the mixture was stirred at 90° C. for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-100% EtOAc in PE) to give the title compound (1.10 g, yield 55.5%) as a yellow oil. ¹H NMR (400 MHz, DMSO-d6) δ 7.78 (s, 1H), 7.72 (d, J=1.7 Hz, 1H), 7.29 (s, 1H), 7.19 (d, J=1.8 Hz, 1H), 4.55 (t, J=5.3 Hz, 1H), 4.44 (d, J=5.3 Hz, 2H), 3.25 (s, 6H). LC/MS (ESI) m/z: 224 (M+H)⁺.

### Step 4: 1-Oxo-1,2-dihydropyrrolo[1,2-a]pyrazine-7-carbonitrile (5)

To a solution of 4-cyano-1-(2,2-dimethoxyethyl)-1H-pyrrole-2-carboxamide (1.10 g, 4.93 mmol) in AcOH (10 mL) and the mixture was stirred at 100° C. for 16 hours. The slurry was filtered, and the filter cake was washed with EtOAc, dried under vacuum to give the title compound (600 mg, yield 76.5%) as a yellow solid, which was used directly in the next step without further purification. ¹H NMR (400 MHz, DMSO-de) δ 10.81 (s, 1H), 8.15 (d, J=1.3 Hz, 1H), 7.35 (d, J=5.9 Hz, 1H), 7.34 (s, 1H), 6.77 (t, J=5.7 Hz, 1H). LC/MS (ESI) m/z: 160 (M+H)⁺.

### Step 5: 1-Bromopyrrolo[1,2-a]pyrazine-7-carbonitrile (6)

To a solution of 1-oxo-1,2-dihydropyrrolo[1,2-a]pyrazine-7-carbonitrile (600 mg, 3.77 mmol) in MeCN (20 mL) was added POBr₃ (2.16 g, 7.54 mmol) at 0° C. and the mixture was stirred at 90° C. for 16 hours. The mixture was filtered, and the filter cake was washed with MeOH, dried under vacuum to give the title compound (400 mg, yield 47.8%) as a yellow solid, which was used directly in the next step without further purification. ¹H NMR (400 MHz, DMSO-d6) δ 8.57 (s, 1H), 8.41 (dd, J=4.8, 0.9 Hz, 1H), 7.51 (d, J=4.8 Hz, 1H), 7.39 (s, 1H). LC/MS (ESI) m/z: 223 (M+H)⁺.

### Step 6: Pyrrolo[1,2-a]pyrazine-7-carbonitrile (7)

To a solution of 1-bromopyrrolo[1,2-a]pyrazine-7-carbonitrile (200 mg, 0.90 mmol) in THF (20 mL) and MeOH (3 mL) was added Pd/C (30 mg, 10% wt.), the mixture was degassed under N₂ atmosphere for three times and stirred under a H₂ balloon at room temperature for 16 hours. The mixture was filtered, and the filtrate was concentrated under reduced pressure to dryness to give the title compound (120 mg, yield 93.1%) as a yellow solid, which was used directly in the next step without further purification. LC/MS (ESI) m/z: 144 (M+H)⁺.

### Step 7: Pyrrolo[1,2-a]pyrazin-7-ylmethanamine (8)

To a solution of pyrrolo[1,2-a]pyrazine-7-carbonitrile (120 mg, 0.84 mmol) in a solution of 7N NH₃/MeOH solution (3 mL) was added Raney Ni (12 mg, wt.), the mixture was degassed under N₂ atmosphere for three times and stirred under a H₂ balloon at room temperature for 3 minutes. The mixture was filtered, and the filtrate was concentrated under reduced pressure to dryness to give the title compound (120 mg, yield 97.3%) as a yellow solid, which was used directly in the next step without further purification. LC/MS (ESI) m/z: 148 (M+H)⁺.

### Step 8: (2S,4R)-4-(difluoromethoxy)-1-((phenoxathiine-3-carbonyl)glycyl)-N-(pyrrolo[1,2-a]pyrazin-7-ylmethyl)pyridine-2-carboxamide (Compound 266)

To a mixture of (2S,4R)-4-(difluoromethoxy)-1-((phenoxathiine-3-carbonyl)glycyl) pyrrolidine-2-carboxylic acid (20 mg, 0.043 mmol) and pyrrolo[1,2-a]pyrazin-7-ylmethanamine (8 mg, 0.052 mmol) in DMF (0.5 mL) was added DIPEA (28 mg, 0.22 mmol) and HAT U (21 mg, 0.056 mmol) under N₂ atmosphere and the reaction mixture was stirred at room temperature for 2 hours. The mixture was concentrated under reduced pressure to dryness. The residue was purified by prep-HPLC to give Compound 266 (1.0 mg, yield 3.9%) as a white solid. ¹H NMR (400 MHz, CD₃OD): δ 8.57 (s, 1H), 7.95 (d, J=4.9 Hz, 1H), 7.62 (s, 1H), 7.51-7.47 (m, 1H), 7.42 (d, J=1.7 Hz, 1H), 7.26-7.20 (m, 2H), 7.19-7.14 (m, 2H), 7.08 (d, J=7.4 Hz, 1H), 7.04 (s, 1H), 6.85 (s, 1H), 6.51 (t, J=74.4 Hz, 1H), 5.05-4.96 (m, 1H), 4.58 (d, J=7.7 Hz, 1H), 4.55 (d, J=6.4 Hz, 2H), 4.19 (s, 2H), 4.03-3.97 (m, 1H), 3.87 (d, J=10.4 Hz, 1H), 2.52-2.45 (m, 1H), 2.29-2.22 (m, 1H). LC/MS (ESI) m/z: 594 (M+H)⁺. RT (Method A): 1.44 min.

### Scheme 74. Synthesis of (1S,3S,5S)-5-methyl-N-((6-methyl-11H-pyrrolo[3,2-c]pyridin-2-yl)methyl)-2-((phenoxathiine-3-carbonyl)glycyl)-2-azabicyclo[3.1.0]hexane-3-carboxamide (Compound 268)

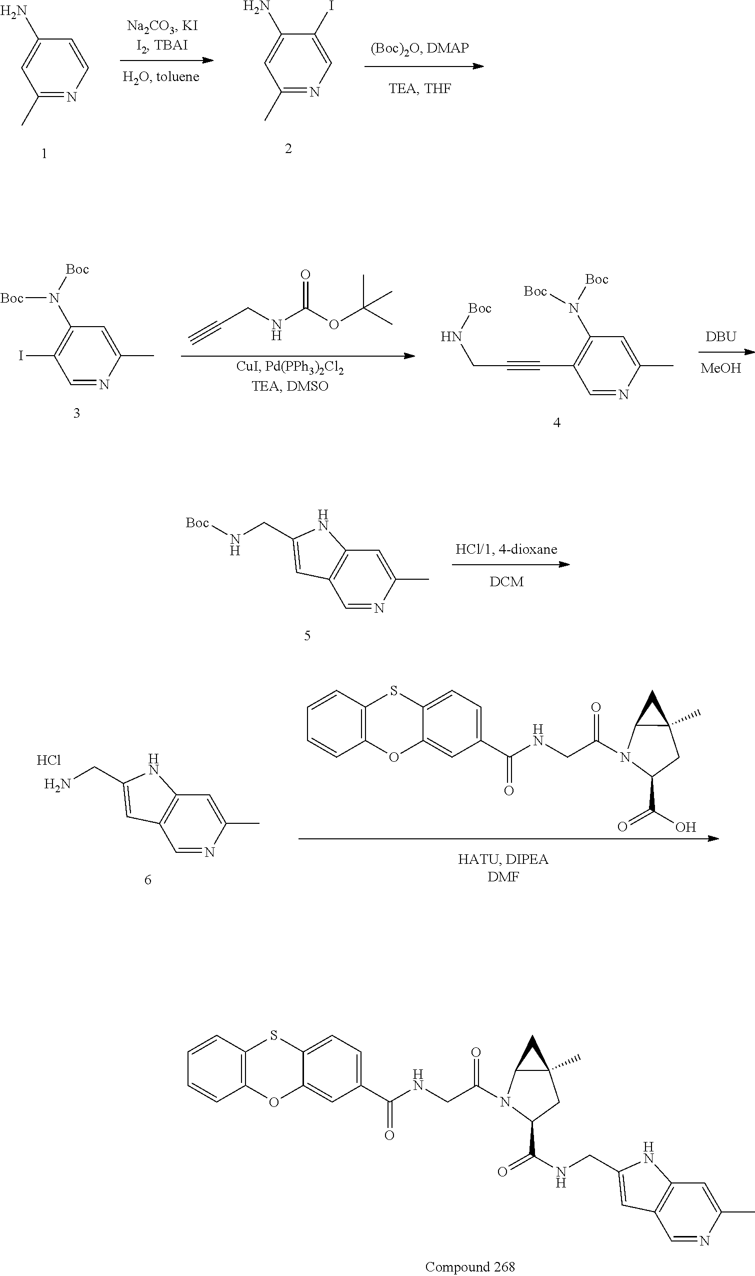

### Step 1: 5-Iodo-2-methylpyridin-4-amine (2)

To a solution of 2-methylpyridin-4-amine (7.5 g, 69.4 mmol) in water (90 mL) was added Na₂CO₃ (5.2 g, 48.6 mmol), KI (15.0 g, 90.2 mmol), 12 (14.1 g, 55.1 mmol) and TBAI (300 mg, 0.69 mmol) under N₂ atmosphere and the reaction mixture was stirred at 100° C. overnight. The mixture was diluted with EtOAc, washed with saturated aq. Na₂S₂O₃ solution and brine, dried over anhydrous Na₂SO₄, filtered, and concentrated under reduced pressure to dryness. The residue was purified by flash chromatography (silica gel, 0-4% MeOH in DCM) to give the title compound (2.0 g, yield 12.3%) as orange solid. LC/MS (ESI) m/z: 235 (M+H)⁺.

### Step 2: Tert-butyl (tert-butoxycarbonyl)(5-iodo-2-methylpyridin-4-yl)carbamate (3)

To a solution of 5-iodo-2-methylpyridin-4-amine (400 mg, 1.71 mmol) in THF (5 mL) was added TEA (863 mg, 8.55 mmol), DMAP (21 mg, 0.17 nmol) and (Boc)₂O (1.12 g, 5.13 mmol) under N₂ atmosphere and the reaction mixture was stirred at 50° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-12% EtOAc in PE) to give the title compound (620 mg, yield 83.6%) as a white solid. LC/MS (ESI) m/z: 435 (M+H)⁺.

### Step 3: Tert-butyl (tert-butoxycarbonyl) (5-(3-((tert-butoxycarbonyl)amino)prop-1-yn-1-yl)-2-methylpyridin-4-yl)carbamate (4)

To a mixture of tert-butyl (tert-butoxycarbonyl)(5-iodo-2-methylpyridin-4-yl)carbamate (2.0 g, 4.76 mmol) and tert-butyl prop-2-yn-1-ylcarbamate (310 mg, 0.71 mmol) in TEA (4 mL) and DMSO (2 mL) was added CuI (7 mg, 0.04 mmol) and Pd(PPh₃)₂Cl₂ (25 mg, 0.04 mmol) under N₂ atmosphere, the mixture was degassed under N₂ atmosphere for ten times and stirred at 50° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-25% EtOAc in PE) to give the title compound (300 mg, yield 90.9%) as a yellow oil. LC/MS (ESI) m/z: 463 (M+H)⁺.

### Step 4: Tert-butyl ((6-methyl-1H-pyrrolo[3,2-c]pyridin-2-yl)methyl)carbamate (5)

To a solution of tert-butyl (tert-butoxycarbonyl)(5-(3-((tert-butoxycarbonyl)amino) prop-1-yn-1-yl)-2-methylpyridin-4-yl)carbamate (300 mg, 0.65 mmol) in MeOH (4 mL) was added DBU (297 mg, 1.95 mmol) and the reaction mixture was stirred at 90° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give the title compound (100 mg, yield 59.1%) as a brown solid. LC/MS (ESI) m/z: 262 (M+H)⁺.

### Step 5: (6-Methyl-1H-pyrrolo[3,2-c]pyridin-2-yl)methanamine hydrochloride (6)

To a solution of tert-butyl ((6-methyl-1H-pyrrolo[3,2-c]pyridin-2-yl)methyl) carbamate (100 mg, 0.38 mmol) in DCM (1 mL) was added HCl/1,4-dioxane (2 mL, 4M) and the reaction mixture was stirred under N₂ atmosphere at room temperature for 2 hours. The reaction mixture was concentrated under reduced pressure to dryness to give the title compound (70 mg, yield 93.3%) as a white solid, which was used directly in the next reaction without further purification. LC/MS (ESI) m/z: 162 (M+H)⁺.

### Step 6: (1S,3S,5S)-5-methyl-N-((6-methyl-1H-pyrrolo[3,2-c]pyridin-2-yl)methyl)-2-((phenoxathiine-3-carbonyl)glycyl)-2-azabicyclo[3.1.0]hexane-3-carboxamide (Compound 268)

To a mixture of (1S,3S,5S)-5-methyl-2-((phenoxathiine-3-carbonyl)glycyl)-2-azabicyclo [3.1.0]hexane-3-carboxylic acid (30 mg, 0.07 mmol) and (6-methyl-1H-pyrrolo[3,2-c]pyridin-2-yl)methanamine hydrochloride (23 mg, 0.14 mmol) in DMF (2 mL) was added DIPEA (55 mg, 0.42 mmol) and HATU (32 mg, 0.08 mmol) under N₂ atmosphere and the reaction mixture was stirred at 25° C. for 2 hours. The mixture was diluted with EtOAc, washed with saturated aq. NH₄Cl solution, dried over anhydrous Na₂SO₄, filtered, and concentrated under reduced pressure to dryness. The residue was purified by prep-TLC (DCM:MeOH=10:1) and further purified by prep-HPLC to give Compound 268 (5 mg, yield 12.5%) as a white solid. ¹H NMR (400 MHz, CD₃OD): δ 8.60 (s, 1H), 8.48 (s, 1H), 7.46 (d, J=8.1 Hz, 1H), 7.31 (s, 2H), 7.21 (s, 1H), 7.15 (d, J=8.2 Hz, 2H), 7.10 (d, J=7.5 Hz, 1H), 6.99 (d, J=8.0 Hz, 1H), 6.70 (s, 1H), 4.91 (d, J=3.7 Hz, 1H), 4.63 (s, 2H), 4.39 (d, J=16.3 Hz, 1H), 4.30 (d, J=16.3 Hz, 1H), 3.45 (m, 1H), 2.52 (s, 3H), 2.47 (d, J=11.9 Hz, 1H), 2.22 (m, 1H), 1.33 (s, 3H), 1.28-1.27 (m, 1H), 0.91 (s, 1H). LC/MS (ESI) m/z: 568 (M+H)⁺. RT (Method A): 1.46 min.

Compound 267 was prepared based on Steps2-6 in Scheme 74:

| Cmpd # | Reactant A | Reactant B | Reactant C | Characterization Data |
|---|---|---|---|---|
| 267 | 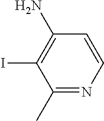 | 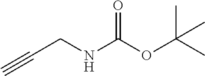 | 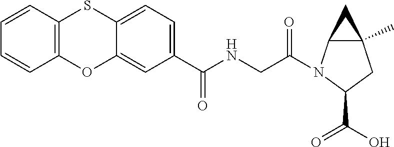 | ¹H NMR (400 MHz, CD₃OD): δ 7.43 (d, J = 6.8 Hz, 1H), 7.30 (d, J = 1.7 Hz, 1H), 7.22-7.19 (m, 1H), 7.15-7.14 (m, 1H), 7.13-7.08 (m, 1H), 7.00 (d, J = 8.0 Hz, 1H), 6.83 (s, 1H), 4.90 (m, 1H), 4.65 (m, 1H), 4.40 (d, J = 16.2 Hz, 1H), 4.27 (d, J = 16.2 Hz, 1H), 3.46 (m, 1H), 2.76 (s, 1H), 2.49 (m, 1H), 2.23 (m, 1H). LC/MS (ESI) m/z: 568 (M + H)⁺. RT (Method A): 1.46 min. |

### Scheme 75. Synthesis of (2S,4R)-N-((1II-pyrrolo[3,2-c]pyridin-2-yl)methyl)-4-(difluoromethoxy)-1-((3-(thiophen-2-yloxy)benzoyl)glycyl)pyrrolidine-2-carboxamide (Compound 275)

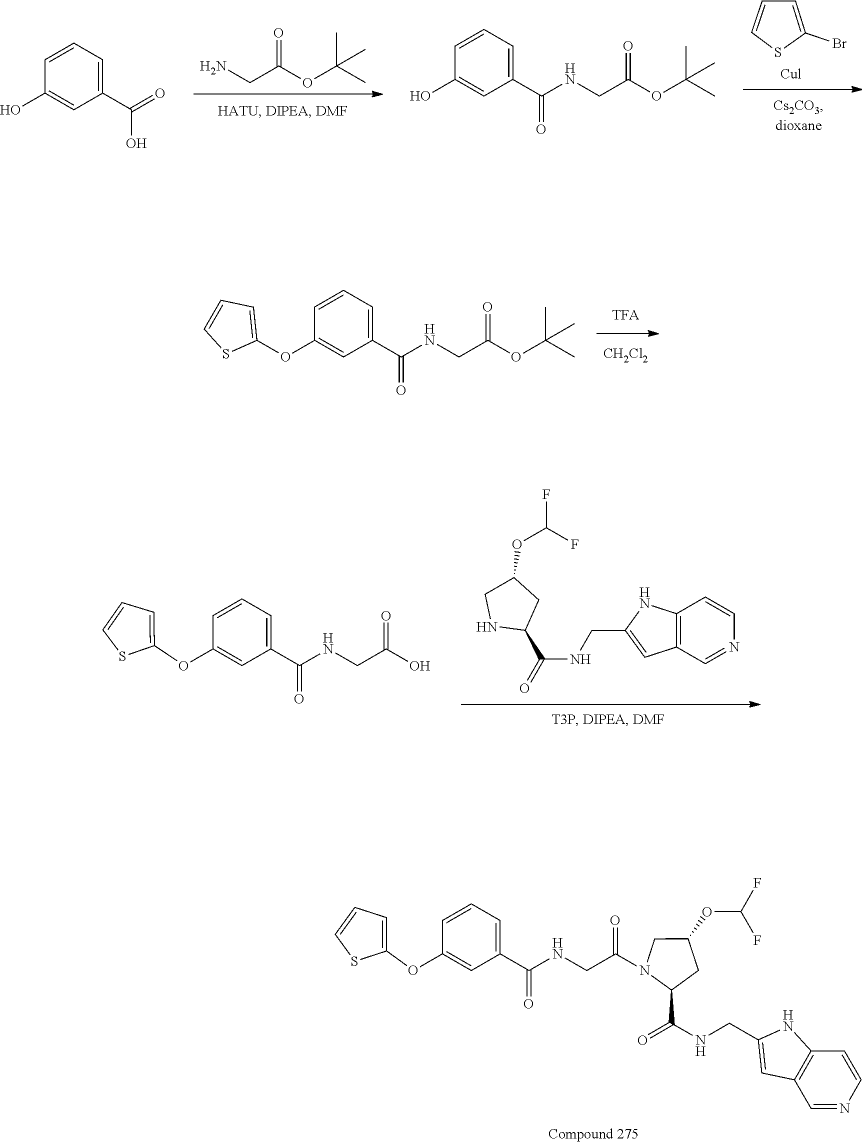

### Step 1: Tert-butyl 2-[(3-hydroxybenzoyl)amino]acetate (2)

To a solution of 3-hydroxybenzoic acid (1.000 g, 7.240 mmol, 1.0 equiv.) in DMF (5 mL) was added glycine tert-butyl ester hydrochloride (1.578 g, 9.413 mmol, 1.2 equiv.), HATU (4.129 g, 10.86 mmol, 1.3 equiv.) and DIPEA (0.406 mL, 2.33 mmol, 3.0 equiv.). The reaction mixture was stirred at room temperature for 30 minutes. Water (5 mL) and EtOAc (5 mL) were added, and the two layers separated. The aq. layer was extracted twice with EtOAc washed with brine, dried over Na₂SO₄, and concentrated. Purification by Combi Flash, 12 g column, solvent A=CH₂Cl₂, solvent B=MeOH, 100% A to 5% B gave tert-butyl 2-[(3-hydroxybenzoyl)amino]acetate (1.535 g, 6.108 mmol, 84.37% yield) as a clear oil. LC/MS (ESI) m/z: 252 (M+H)⁺.

### Step 2: methyl (S)-7-(2-(4-bromophenyl)acetyl)-1,4-dioxa-7-azaspiro[4.4]nonane-8-carboxylate (3)

A mixture of tert-butyl 2-[(3-hydroxybenzoyl)amino]acetate (150.0 mg, 0.5969 mmol, 1.0 equiv.), 2-bromothiophene (0.1946 g, 1.194 mmol, 2 equiv.), Copper(I) iodide (11.37 mg, 0.05970 mmol, 0.1 equiv.), N,N-dimethylglycine hydrochloride (16.66 mg, 0.1194 mmol, 0.2 equiv.), cesium carbonate (0.7780 g, 2.388 mmol, 4 equiv.) in 1,4-dioxane (3 mL) was stirred in a microwave reactor at 110° C. for 12 h. The reaction mixture was cooled to room temperature, diluted with water, extracted with EtOAc, washed with brine, dried over Na₂S₀₄ and concentrated. Purification by combi-Flash; 12 g column, solvent A=hexanes, solvent B=EtOAc. 100% A to 15% B gave tert-butyl 2-[[3-(2-thienyloxy)benzoyl]amino]acetate (30.00 mg, 0.08998 mmol, 15.07% yield). LC/MS (ESI) m/z: 334 (M+H)⁺.

### Step 3: (3-(thiophen-2-yloxy)benzoyl)glycine (4)

TFA (0.5 mL) was added to a solution of tert-butyl 2-[[3-(2-thienyloxy)benzoyl]amino]acetate (25.00 mg, 0.07499 mmol, 1.0 equiv.) in CH₂Cl₂ (1 mL) at ice-bath temperature. The reaction mixture was stirred at room temperature for 2 hours and then concentrated to dryness to give 2-[[3-(2-thienyloxy)benzoyl]amino]acetic acid (20.50 mg, 0.07393 mmol, 98.59% yield) as a white solid which was used in the next step without further purification. LC/MS (ESI) m/z: 278 (M+H)⁺.

### Step 4: (S)-N-((R)-1-(4-carbamimidoylthiophen-2-yl)ethyl)-7-(2-(2′,4′-difluoro-[, 1′-biphenyl]-4-yl)acetyl)-1,4-dioxa-7-azaspiro[4.4]nonane-8-carboxamide (Compound 275)

To a solution of 2-[[3-(2-thienyloxy)benzoyl]amino]acetic acid (6.397 mg, 0.02307 mmol, 1.0 equiv.) in DMF (2 mL) was added (2S,4R)-4-(difluoromethoxy)-N-(1H-pyrrolo[3,2-c]pyridin-2-ylmethyl)pyrrolidine-2-carboxamide;hydrochloride (8.000 mg, 0.02307 mmol, 1.0 equiv.), T3P, (50 mass %) in ethyl acetate (20.7 μL, 0.0346 mmol, 1.5 equiv.), and DIPEA (12.1 μL, 0.0694 mmol, 3.0 equiv.). The reaction mixture was stirred at room temperature for 30 minutes and then purified by directly prep HPLC to give Compound 275 (2.400 mg, 0.004213 mmol, 18.27% yield). LC/MS (ESI) m/z: 570 (M+H)⁺. ¹H NMR (400 MHz, DMSO-d6): δ 11.15 (s, 1H), 8.73 (d, J=15.7 Hz, 1H), 8.63 (d, J=15.6 Hz, 1H), 8.11 (s, 1H), 8.03 (d, J=5.7 Hz, 1H), 7.96 (d, J=5.7 Hz, 1H), 7.71-7.52 (m, 2H), 7.51-7.34 (m, 1H), 7.24 (dd, J=8.3, 2.9 Hz, 2H), 7.10 (d, J=5.7 Hz, 1H), 7.09-6.99 (m, 1H), 6.84 (dd, J=5.8, 3.8 Hz, 1H), 6.73 (d. J=14.6 Hz, 1H), 6.68-6.60 (m, 1H), 6.60-6.42 (m, 1H), 6.32 (s, 1H), 4.49-4.21 (m, 2H), 4.13 (dd, J=16.8, 5.8 Hz, 1H), 3.95 (dd, J=16.8, 5.7 Hz, 1H), 3.90-3.67 (m, 3H), 2.30-2.14 (m, 2H), 2.09 (dt, J=13.2, 6.5 Hz, 1H), 1.51-1.19 (m, 1H). RT (Method A): 1.28 min.

### Scheme 76. Synthesis of (S)-7-((phenoxathiine-3-carbonyl)glycyl)-N-(pyrrolo[1,2-c]pyrimidin-6-ylmethyl)-1,4-dioxa-7-azaspiro[4.4]nonane-8-carboxamide (Compound 276)

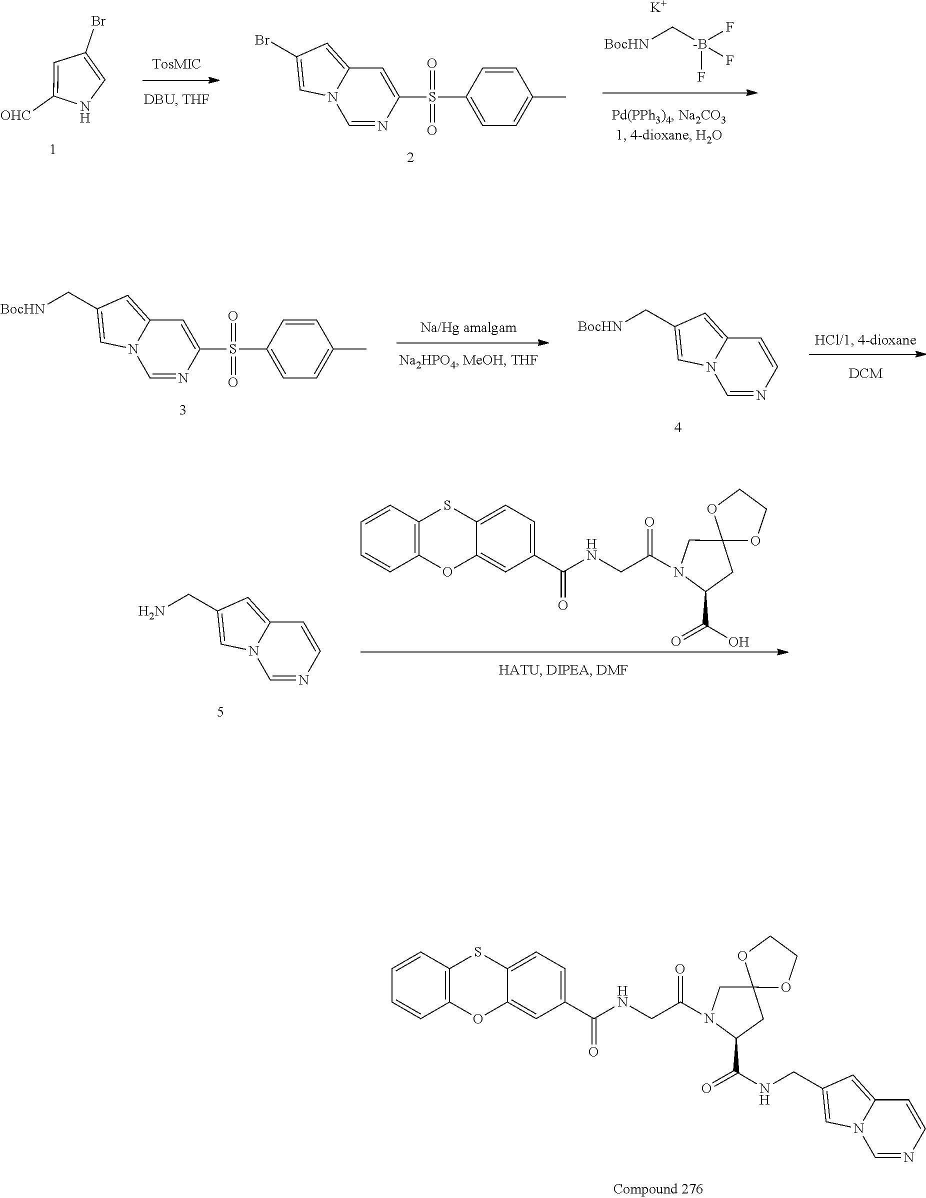

### Step 1: 6-Bromo-3-tosylpyrrolo[1, 2-c]pyrimidine (2)

To a mixture of 4-bromo-1H-pyrrole-2-carbaldehyde (3.0 g, 17.3 mmol) and DBU (2.9 g, 26.0 mmol) in THE (40 mL) was added tosylmethyl isocyanide (3.8 mg, 26.0 mmol) under N₂ atmosphere and die reaction mixture was stirred at room temperature for 2 hours. The mixture was acidified with AcOH to pH˜6 and extracted with EtOAc twice, washed with water, dried over anhydrous Na₂SO₄, filtered, and concentrated under reduced pressure to dryness. The residue was purified by flash chromatography (silica gel, 0-60% DCM in PE) to give the title compound (3.4 g, yield 56.7%) as a brown solid. LC/MS (ESI) m/z: 351/353 (M+H)⁺.

### Step 2: Tert-butyl ((3-tosylpyrrolo[1,2-c]pyrimidin-6-yl)methyl)carbamate (3)

To a mixture of 6-bromo-3-tosylpyrrolo[1,2-c]pyrimidine (400 mg, 1.14 mmol) and potassium {[(tert-butoxycarbonyl)amino]methyl}trifluoroborate (325 mg, 1.37 mmol) in 1,4-dioxane (6 mL) and water (1 mL) was added Na₂CO₃ (363 mg, 3.42 mmol) and Pd(PPh₃)₄ (132 mg, 0.11 mmol) under N₂ atmosphere, the reaction mixture was degassed under N₂ atmosphere for three times and stirred at 100° C. for 16 hours. The mixture was diluted with EtOAc, washed with water, dried over anhydrous Na₂SO₄, filtered, and concentrated under reduced pressure to dryness. The residue was purified by flash chromatography (silica gel, 0-30% EtOAc in PE) to give the title compound (300 ng, yield 65.5%) as a brown solid. LC/MS (ESI) n/z: 402 (M+H)⁺.

### Step 3: Tert-butyl (pyrrolo[1,2-c]pyrimidin-6-ylmethyl)carbamate (4)

To a mixture of Na₂HPO₄ (300 mg, 3.00 mmol) and Na/Hg-amalgam (172 mg, 1.50 mmol, 20% wt.) in MeOH (6 mL) was added the solution of tert-butyl ((3-tosylpyrrolo[1,2-c]pyrimidin-6-yl)methyl)carbamate (50 mg, 0.75 mmol) in THE (6 mL) at 0° C. under N₂ atmosphere and the reaction mixture was stirred at room temperature for 2 hours. The mixture was diluted with EtOAc and washed with brine, dried over anhydrous Na₂SO₄, filtered, and concentrated under reduced pressure to dryness. The residue was purified by flash chromatography (silica gel, 0-5% MeOII in DCM) to give the title compound (174 mg, yield 96.7%) as a brown solid. LC/MS (ESI) m/z: 248 (M+H)⁺.

### Step 4: Pyrrolo[1,2-c]pyrimidin-6-ylmethanamine (5)

To a solution of tert-butyl (pyrrolo[1,2-c]pyrimidin-6-ylmethyl)carbamate (80 mg, 0.32 mmol) in DCM (1 mL) was added HCl/1,4-dioxane (3 mL, 4M) and the reaction mixture was stirred at room temperature for 2 hours. The mixture was concentrated under reduced pressure to dryness to give the title compound (45 mg, yield 6.5%) as a brown oil. Which was used directly in the next step without further purification. LC/MS (ESI) m/z: 148 (M+H)⁺.

### Step 5: (S)-7-((phenoxathiine-3-carbonyl)glycyl)-N-(pyrrolo[1,2-c]pyrimidin-6-ylmethyl)-1,4-dioxa-7-azaspiro[4.4]nonane-8-carboxamide (Compound 276)

To a mixture of (S)-7-((phenoxathiine-3-carbonyl)glycyl)-1,4-dioxa-7-azaspiro[4.4]nonane-8-carboxylic acid (30 mg, 0.066 mol) and pyrrolo[1,2-c]pyrimidin-6-ylmethanamine hydrochloride (19 mg, 0.13 mmol) in DMF (3 mL) was added DIPEA (51 mg, 3.96 mmol) and HATU (38 mg, 0.10 mol) under N₂ atmosphere and the reaction mixture was stirred at room temperature for 1 hour. The mixture was diluted with EtOAc, washed with saturated aq. NH₄Cl solution, dried over anhydrous Na₂SO₄, filtered, and concentrated under reduced pressure to dryness. The residue was purified by prep-TLC (DCM:MeOH=15:1) and further purified by prep-HPLC to give Compound 276 (15 mg, yield 39.5%) as a white solid. ¹H NMR (400 MHz, CD₃OD): δ 8.76 (s, 1H), 7.45 (s, 1H), 7.44-7.41 (m, 1H), 7.33 (d, J=1.7 Hz, 1H), 7.21-7.17 (m, 1H), 7.15-7.10 (m, 3H), 7.09-7.06 (m, 2H), 7.02 (d, J=8.0 Hz, 1H), 6.39 (s, 1H), 4.65-4.62 (m, 1H), 4.60 (d, J=9.3 Hz, LH), 4.45 (d, J=15.7 Hz, 1H), 4.18 (d, J=16.3 Hz, 1H), 4.07 (d, J=16.3 Hz, 1H), 4.01 (s, 4H), 3.86 (d, J=10.8 Hz, LH), 3.80 (d, J=10.7 Hz, 1H), 2.49-2.42 (m, 1H), 2.29-2.22 (m, 1H). LC/MS (ESI) m/z: 586 (M+H)⁺. RT (Method A): 1.82 min.

### Scheme 77. Synthesis of (2S,4R)-N-(benzo[d]oxazol-5-ylmethyl)-4-(difluoromethoxy)-1-((phenoxathiine-3-carbonyl)glycyl)pyrrolidine-2-carboxamide (Compound 282)

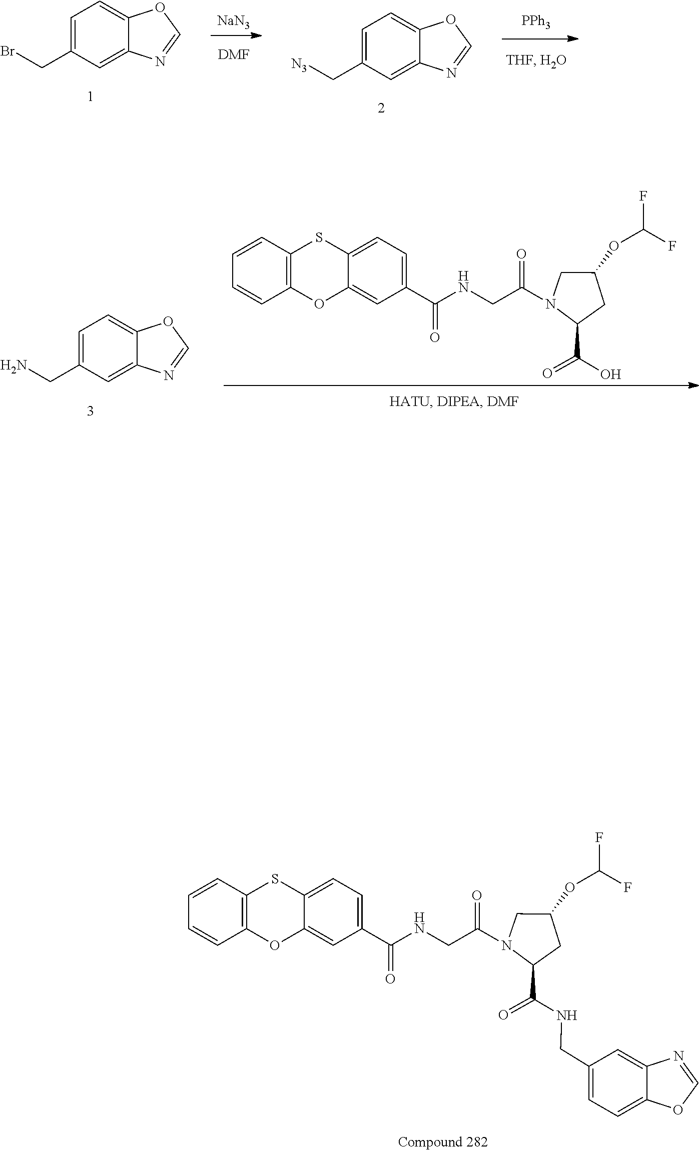

### Step 1: 5-(Azidomethyl)benzo[d]oxazole (2)

To a solution of 5-(bromomethyl)benzo[d]oxazole (50 mg, 0.24 mmol) in DMF (3 mL) was added NaN₃ (77 mg, 1.18 mmol) and the reaction mixture was stirred at 60° C. for overnight. The mixture was diluted with EtOAc, washed with saturated aq. NH₄Cl solution, dried over anhydrous Na₂SO₄, filtered, and concentrated under reduced pressure to dryness. The residue was purified by flash chromatography (silica gel, 0-25% EtOAc in PE) to give the title compound (30 mg, yield 41.0%) as a colorless oil. LC/MS (ESI) m/z: 175 (M+H)⁺.

### Step 2: Benzo[d]oxazol-5-ylmethanamine (3)

To a solution of 5-(azidomethyl)benzo[d]oxazole (30 mg, 0.17 mmol) in THF (3 mL) and water(1 mL) was added PPh₃ (60 mg, 0.23 mmol) and the reaction mixture was stirred at room temperature overnight. The mixture was diluted with EtOAc, washed with brine, dried over anhydrous Na₂SO₄, filtered, and concentrated under reduced pressure to dryness to give the title compound (50 mg, crude) as a yellow oil, which was used directly in the next step without further purification. LC/MS (ESI) m/z: 149 (M+H)⁺.

### Step 3: (2S,4R)-N-(benzo[d]oxazol-5ylmethyl)-4-(difluoromethoxy)-1-((phenoxathiine-3-carbonyl)glycyl)pyrrolidine-2-carboxamide (Compound 282)

To a mixture of (2S,4R)-4-(difluoromethoxy)-1-((phenoxathiine-3-carbonyl)glycyl) pyrrolidine-2-carboxylic acid (40 mg, 0.08 mmol) and benzo[d]oxazol-5-ylmethanamine (50 mg, 0.33 mmol, crude) in DMF (2 mL) was added DIPEA (43 mg, 0.48 mmol) and HATU (67 mg, 0.12 mmol) under N₂ atmosphere and the reaction mixture was stirred at room temperature for 2 hours. The mixture was diluted with EtOAc, washed with saturated aq. NH₄Cl solution, dried over anhydrous Na₂SO₄, filtered, and concentrated under reduced pressure to dryness. The residue was purified by prep-TLC (DCM:MeOH=11:1) and further purified by prep-HPLC to give Compound 282 (1.4 mg, yield 2.7%) as a white solid. ¹H NMR (400 MHz, CD-OD): δ 8.39 (s, 1H), 7.70 (s, 1H), 7.55 (d, J=8.4 Hz, 1H), 7.49 (d, J=8.0 Hz. 1H), 7.43-7.39 (m, 2H), 7.22-7.17 (m, 2H), 7.13 (s, 1H), 7.08 (d, J=7.6 Hz, 1H), 7.03 (d, J=8.1 Hz, 1H), 6.51 (s, 1H), 5.01 (s, 1H), 4.60 (t, J=7.7 Hz, 2H), 4.24 (d, J=16.7 Hz, 1H), 4.12 (d, J=16.7 Hz, 1H), 4.00-3.95 (m, 1H), 3.87 (s, 1H), 3.85-3.74 (m, 1H), 2.48 (s, 1H), 2.27-2.21 (m, 1H). LC/MS (ESI) m/z: 595 (M+H)⁺. RT (Method A): 1.99 min.

### Scheme 78. Synthesis of N-[2-[(2S,4R)-4-(difluoromethoxy)-2-[[(1R)-1-(1H-pyrrolo[3,2-c]pyridin-2-yl)ethyl]carbamoyl]pyrrolidin-1-yl]-2-oxo-ethyl]-[1,4]benzoxathiino[3,2-b]pyridine-3-carboxamide (Compound 283)

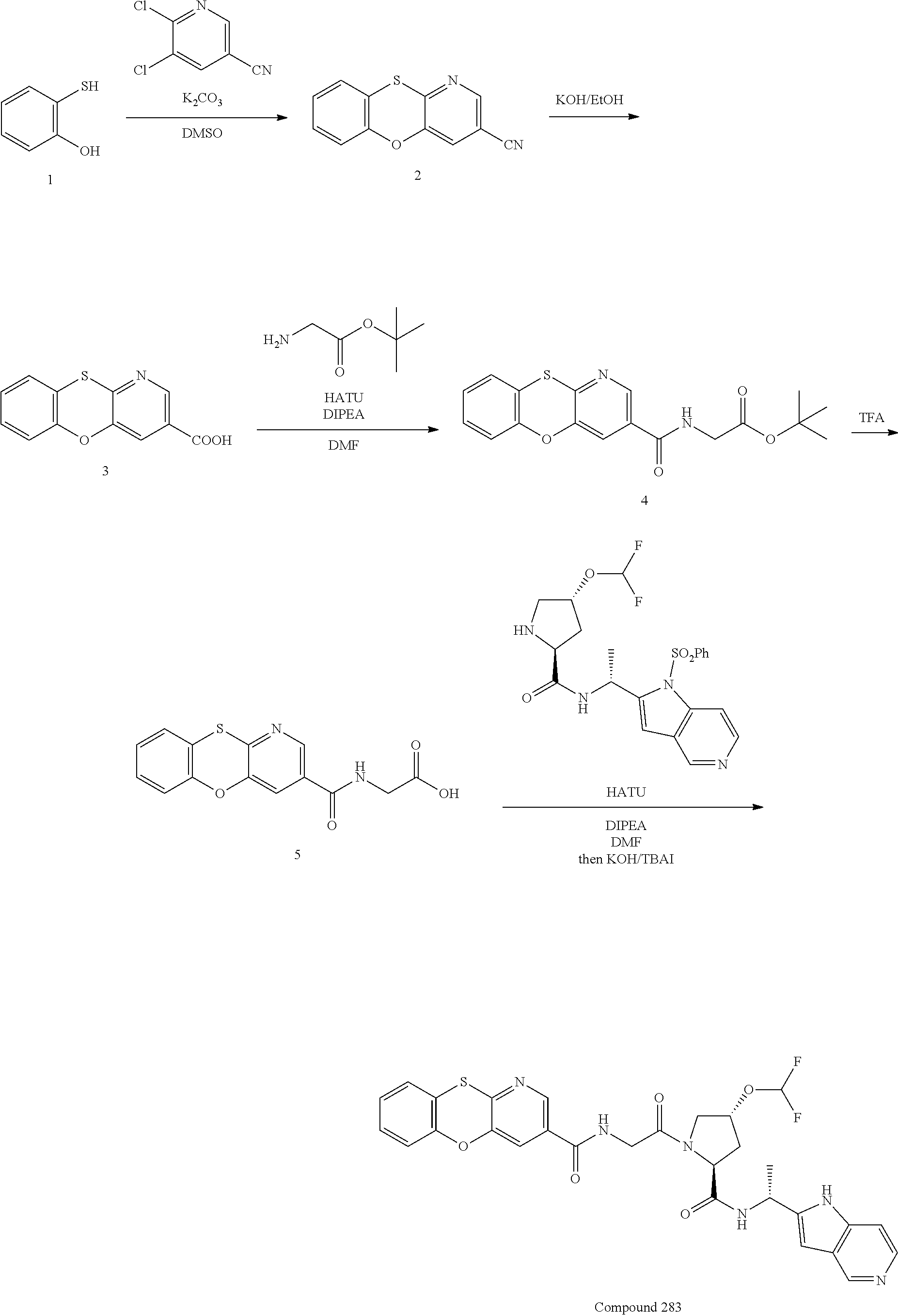

### Step 1: [1,4]benzoxathiino[3,2-b]pyridine-3-carbonitrile

To a mixture of 2-sulfanylphenol (0.12 mL, 1.2 mmol) and 5,6-dichloropyridine-3-carbonitrile (200 mg, 1.12 mmol) in DMSO (2 mL) was added K₂CO₃ (0.48 g, 3.5 mmol) at room temperature. The reaction mixture was stirred for 48 hours. The reaction mixture was diluted with water and stirred for 10 minutes. The solid was filtered and purified on silica gel (0 to 30% ethyl acetate in hexanes) to give [1,4]benzoxathiino[3,2-b]pyridine-3-carbonitrile (160 mg, 61.2% yield) as an off-white solid.

### Step 2: [1,4]benzoxathiino[3,2-b]pyridine-3-carboxylic acid

A solution of [1,4]benzoxathiino[3,2-b]pyridine-3-carbonitrile (160 mg, 0.71 mmol) and NaOH (0.11 g, 2.8 mmol) in 70% ethanol in water was heated to reflux for 2 hours. LC/MS shown full conversion, but majority in amide form. The reaction mixture was adjusted with 1 N HCl to pH 2. The solid was filtered and suspended in 9 to 1 dioxane and water, 1 N HCl (0.7 mL) was added, followed by TiCl₄ (0.008 mL, 0.07 mmol). The reaction mixture was heated to reflux for 3 hours. LC/MS shown 90% conversion to desired carboxylic acid. The reaction mixture was diluted with water, and the solid was filtered to give [1,4]benzoxathiino[3,2-b]pyridine-3-carboxylic acid (220 mg, >theory) as a yellow solid. This material was used in next step without further purification. LC/MS (ESI) m/z: 426 (M+H)⁺.

### Step 3: tert-butyl 2-(phenoxathiine-3-carbonylamino)acetate

To a suspension of [1,4]benzoxathiino[3,2-b]pyridine-3-carboxylic acid (220 mg, 0.9 mmol), tert-butyl 2-aminoacetate (0.14 g, 1.1 mmol) and HATU (0.5 g, 1 mmol) in DMF (5 mL) was added DIPEA (0.8 mL, 5 mmol) at room temperature. The reaction mixture was stirred at room temperature for 30 minutes. Water was added, and the reaction mixture was extracted with ethyl acetate for three times. The combined organic layers were washed with saturated brine, Na₂SO₄, filtered, and concentrated to give tert-butyl 2-(phenoxathiine-3-carbonylamino)acetate (320 mg, theory). This material was used in next step without further purification. LC/MS (ESI) m/z: 359 (M+H)⁺.

### Step 4: 2-(Phenoxathine-3-carbonylamino)acetic acid

To a solution of tert-butyl 2-(phenoxathiine-3-carbonylamino)acetate (320 mg, 0.9 mmol) in DCM (2 mL) was added TFA (2 mL) at room temperature. After 18 hours, the solvent was evaporated, and the crude mixture was azeotroped with 1 N HCl to give 2-(phenoxathiine-3-carbonylamino)acetic acid (270 mg, theory). This material was used in next step without further purification. LC/MS (ESI) m/z: 303 (M+H)⁺.

### Step 5: N-[2-[(2S,4R)-4-(difluoromethoxy)-2-[[(1R)-1-(H-pyrrolo[3,2-c]pyridin-2-yl)ethyl]carbamoyl]pyrrolidin-1-yl]-2-oxo-ethyl]-[1,4]benzoxathiino[3,2-b]pyridine-3-carboxamide (Compound 283)

To a solution of 2-(phenoxathiine-3-carbonylamino)acetic acid (15 mg, 0.05 mmol), HATU (29 mg, 0.076 mmol), and (2S,4R)-N-[(1R)-1-[1-(benzenesulfonyl)pyrrolo[3,2-c]pyridin-2-yl]ethyl]-4-(difluoromethoxy)pyrrolidine-2-carboxamide (23 mg, 0.05 mmol) in DMF (1 mL) was added DIPEA (0.04 mL, 0.2 mmol) at room temperature. The reaction mixture was stirred at room temperature for 1 hour and diluted with water. The crude mixture was extracted with ethyl acetate for three times. The combined organic layers were washed with saturated brine, dried over Na₂SO₄, filtered, and concentrated to give a crude product. The material was diluted with a mixture of THE (0.6 mL) and water (0.2 mL). To this solution was added KOH (5.6 mg, 0.10 mmol) and tetrabutylammonium iodide (0.4 mg) at room temperature. The two-layer mixture was heated to 65° C. for 24 hours. The volatiles were removed by concentration under reduced pressure. The mixture was extracted with ethyl acetate twice and the organic layer were discarded. The aqueous layer was acidified with 1 N HCl to pH 2 and purified by prep-HPLC to give Compound 283 (1 mg, 8.2% yield) as a white solid. ¹H-NMR (400 MHz, DMSO-d6): δ 11.08 (s, 1H), 8.91 (m, 2H), 8.63 (m, 2H), 8.39 (s, 1H), 8.12-7.59 (m, 3H), 7.22 (dt, J=14.9, 7.7 Hz, 2H), 7.09 (t, J=9.1 Hz, 1H), 6.99-6.74 (m, 2H), 6.38 (m, 1H), 5.21-5.00 (m, 1H), 4.87 (m, 1H), 4.38 (t, J=7.7 Hz, 2H), 1.43 (m, 2H), 1.23 (m, 3H). LC/MS (ESI) m/z: 609 (M+H)⁺. RT (Method A): 1.33 min.

### Scheme 79. Synthesis of N-(2-((2S,4R)-2-(((1H-pyrrolo[3,2-c]pyridin-2-yl)methyl)carbamoyl)-4-(difluoromethoxy)pyrrolidin-1-yl)-2-oxoethyl)benzo[5,6][1,4]oxathiino[3,2-b]pyridine-3-carboxamide (Compound 285)

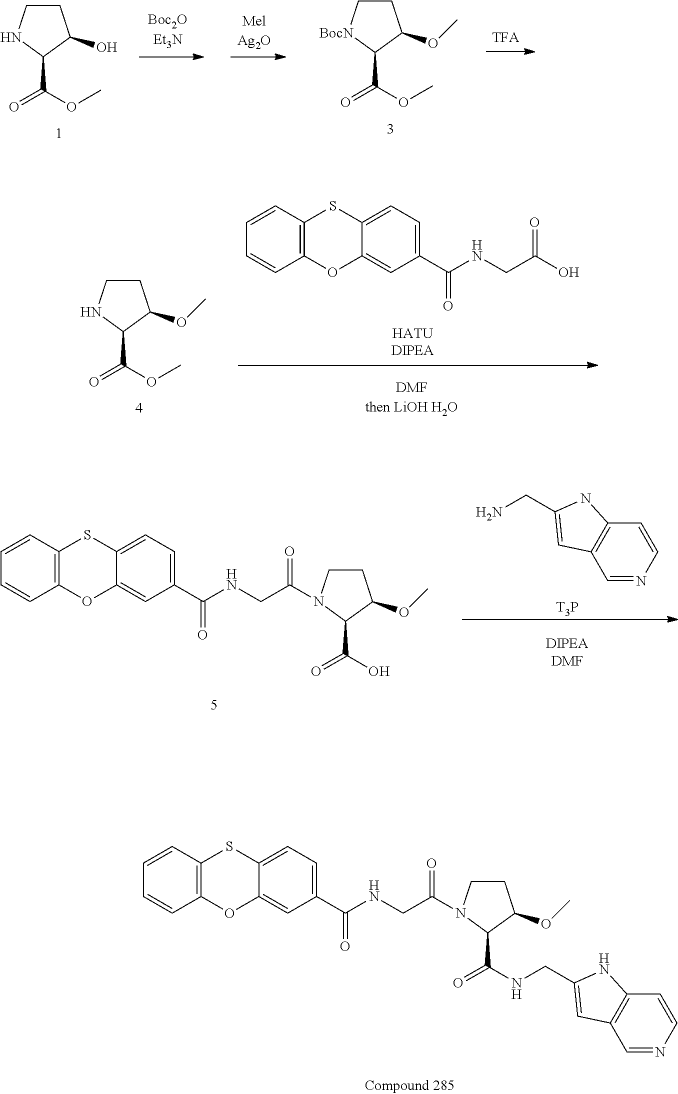

### Step 1: O1-tert-Butyl O2-methyl (2S,3R)-3-methoxypyrrolidine-1,2-dicarboxylate

To a mixture of methyl (2S,3R)-3-hydroxypyrrolidine-2-carboxylate (70 mg, 0.48 mmol) and Boc₂O (0.1 mL, 0.5 mmol) in DCM (2 mL) was added DIPEA (0.25 mL, 1.4 mmol) at room temperature. The reaction mixture was stirred at room temperature for 24 hours. Water was added and the reaction mixture was extracted with ethyl acetate for three times. The organic layers were washed with saturated brine, dried over Na₂SO₄, filtered, and concentrated to give a red oil. This material was treated with Ag₂O (167 mg, 0.27 mmol) and MeI (0.05 mL, 0.8 mmol) in acetone (5 mL) in dark for 48 hours. The solid was filtered and the filtrate was concentrated and purified on silica gel (0 to 50% ethyl acetate in hexanes) to give O1-tert-butyl O2-methyl (2S,3R)-3-methoxypyrrolidine-1,2-dicarboxylate (70 mg, 56% yield) as a yellow oil. LC/MS (ESI) m/z: 260 (M+H)⁺.

### Step 2: ethyl (2S,3R)-3-methoxypyrrolidine-2-carboxylate hydrochloride

To a solution of O₁-tert-butyl 02-methyl (2S,3R)-3-methoxypyrrolidine-1,2-dicarboxylate (70 mg, 0.28 mmol) in DCM (1 mL) was added TFA (1 mL) at room temperature. The reaction mixture was stirred at room temperature for 3 hours. The volatiles were removed by concentration and azeotroped with 1 N HCl to give methyl (2S,3R)-3-methoxypyrrolidine-2-carboxylate hydrochloride (100 mg, >theory) as a yellow oil. This material was used in next step without further purification. LC/MS (ESI) m/z: 160 (M+H)⁺.

### Step 3: (2S,3R)-3-Methoxy-1-[2-(phenoxathiine-3-carbonylamino)acetyl]pyrrolidine-2-carboxylic acid

To a mixture of 2-(phenoxathiine-3-carbonylamino)acetic acid (78 mg, 0.26 mmol), methyl (2S,3R)-3-methoxypyrrolidine-2-carboxylate hydrochloride (95 mg, 0.26 mmol), and HATU (147 mg, 0.39 mmol) in DMF (2 mL) was added DIPEA (0.18 mL, 1.04 mmol) at room temperature. The mixture was stirred at room temperature for 30 minutes and diluted with water (10 mL). The solid was filtered and dissolved in methanol (1 mL), THF (1 mL) and water (1 mL). To this solution was added LiOH·H₂O (31 mg, 1.29 mmol) and the reaction mixture was stirred at room temperature for 18 hours. The crude mixture was acidified with 1 N HCl to pH 3. The solid was removed by filtration. The filtrate was directly purified by prep-HPLC to give (2S,3R)-3-methoxy-1-[2-(phenoxathiine-3-carbonylamino)acetyl]pyrrolidine-2-carboxylic acid (7 mg, 6.3% yield) as a yellow wax. LC/MS (ESI) m/z: 429 (M+H)⁺.

### Step 4: (2S,3R)-3-methoxy-1-[2-(phenoxathiine-3-carbonylamino)acetyl]-N-(JH-pyrrolo[3,2-c]pyridin-2-ylmethyl)pyridine-2-carboxamide (Compound 285)

To a solution of (2S,3R)-3-methoxy-1-[2-(phenoxathiine-3-carbonylamino)acetyl]pyrrolidine-2-carboxylic acid (7 mg, 0.016 mmol), 1H-pyrrolo[3,2-c]pyridin-2-ylmethanamine (2.4 mg, 0.016 mmol), and DIPEA (0.12 mL, 0.69 mmol) in DMF (1 mL) was added T₃P in 50% ethyl acetate (0.02 mL, 0.035 mmol) at room temperature. The mixture was stirred at room temperature for 1 hour and quenched with water. The reaction mixture was filtered, and the aqueous layer was diluted with DMSO and purified on prep-HPLC to give Compound 285 (1 mg, 11% yield). ¹H-NMR (400 MHz, DMSO-d6). δ 8.76-8.65 (m, 1H), 8.62 (s, 1H), 8.39 (s, 2H), 7.98 (d, J=5.6 Hz, 1H), 7.53 (d, J=7.4 Hz. 1H), 7.31 (d, J=7.9 Hz, 1H), 7.20 (dd, J=16.7, 7.6 Hz, 2H), 7.07 (d, J=7.5 Hz, 1H), 6.77 (s, 3H), 6.30 (s, 1H), 4.35 (s, 3H), 4.07 (m, 2H), 1.5 (m, 2H). LC/MS (ESI) m/z: 558.2 (M+H)⁺. RT (Method A): 1.29 min.

### Scheme 80. Synthesis of (S)-N-((1H-pyrrolo[3,2-c]pyridin-2-yl)methyl)-7-((5-(3,4-dichlorophenoxy) furan-2-carbonyl)glycyl)-1,4-dioxa-7-azaspiro[4.4]nonane-8-carboxamide (Compound 287)

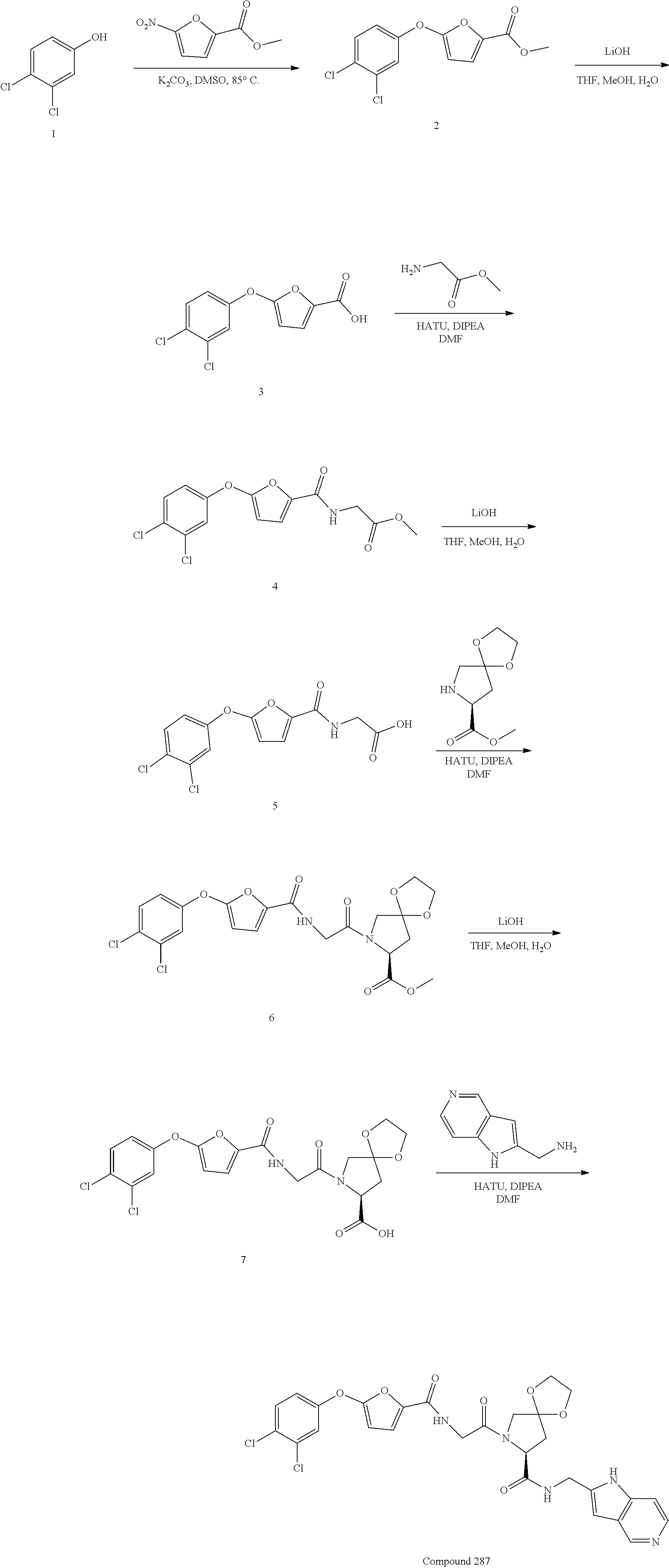

### Step 1: Synthesis of methyl 5-(3,4-dichlorophenoxy)furan-2-carboxylate (2)

To a solution of methyl 5-nitrofuran-2-carboxylate (0.49 g, 3 mmol) in DMSO (5 mL) was added 3,4-dichlorophenol (0.5 g, 3 mmol) and K₂CO₃ (496 mg, 3.6 mmol) and the reaction mixture was stirred at room temperature for 16 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (420 mg, yield 48.9%) as a white solid. LC/MS (ESI) m/z: 287(M+H)⁺.

### Step 2: 5-(3,4-Dichlorophenoxy)furan-2-carboxylic acid (3)

To a solution of methyl 5-(3,4-dichlorophenoxy)furan-2-carboxylate (0.42 g, 1.47 mmol) in THF/MeOH/H₂O (6 mL, 4/1/1) was added LiOH·H₂O (123 mg, 2.93 mmol) under N₂ atmosphere and the reaction mixture was stirred at room temperature for 2 hours. The mixture was acidified with IN aq. HCl to pH˜3 and extracted with EtOAc twice. The combined organic layers were dried over anhydrous Na₂₅₀₄, filtered, and concentrated under reduced pressure to give the title compound (0.39 g, yield 97%) as a white solid, which was used directly in the next step without further purification. LC/MS (ESI) n/z: 273 (M+H)⁺.

### Step 3: Methyl (5-(3,4-dichlorophenoxy)furan-2-carbonyl)glycinate (4)

To a mixture of 5-(3,4-dichlorophenoxy)furan-2-carboxylic acid (390 mg, 1.43 mmol) and methyl glycinate (127 mg, 1.43 mmol) in DMF (5 mL) was added DIPEA (922 mg, 7.15 mmol) and HATU (596 mg, 1.57 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:McOH=15:1) to give the title compound (260 mg, yield 53%) as a yellow solid. LC/MS (ESI) m/z: 344 (M+H)⁺.

### Step 4: (5-(3,4-Dichlorophenoxy)furan-2-carbonyl)glycine (5)

To a solution of methyl (5-(3,4-dichlorophenoxy)furan-2-carbonyl)glycinate (260 mg, 0.75 mmol) in THF/MeOH/H₂O (6 mL, 4/1/1) was added LiOH·H₂O (63 mg, 1.5 mmol) under N₂ atmosphere and the reaction mixture was stirred at room temperature for 2 hours. The mixture was acidified with IN aq. HCl to pH˜3 and extracted with EtOAc twice. The combined organic layers were dried over anhydrous Na₂SO₄, filtered, and concentrated under reduced pressure to give the title compound (240 mg, yield 97%) as a white solid, which was used directly in the next step without further purification. LC/MS (ESI) m/z: 330 (M+H)⁺.

### Step 5: Methyl (S)-7-((5-(3,4-dichlorophenoxy)furan-2-carbonyl)glycyl)-1,4-dioxa-7-azaspiro[4.4]nonane-8-carboxylate (6)

To a mixture of (5-(3,4-dichlorophenoxy)furan-2-carbonyl)glycine (240 mg, 0.73 mmol) and methyl (S)-1,4-dioxa-7-azaspiro[4.4]nonane-8-carboxylate (136 mg. 0.73 mmol) in DMF (3 mL) was added DIPEA (470 mg, 3.65 mmol) and HATU (330 mg, 0.87 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=15:1) to give the title compound (180 mg, yield 49.5%) as a yellow solid. LC/MS (ESI) m/z: 499 (M+H)⁺.

### Step 6: (S)-7-((5-(3,4-dichlorophenoxy)furan-2-carbonyl)glycyl)-1,4-dioxa-7-azaspiro [4.4]nonane-8-carboxylic acid (7)

To a solution of methyl (S)-7-((5-(3,4-dichlorophenoxy)furan-2-carbonyl)glycyl)-1,4-dioxa-7-azaspiro[4.4]nonane-8-carboxylate (180 mg, 0.36 mmol) in THF/MeOH/H₂O (6 mL, 4/1/1) was added LiOH H₂O (30 mg, 0.72 mmol) under N₂ atmosphere and the reaction mixture was stirred at room temperature for 2 hours. The mixture was acidified with 1N aq. HCl to pH˜3 and extracted with EtOAc twice. The combined organic layers were dried over anhydrous Na₂SO₄, filtered, and concentrated under reduced pressure to give the title compound (72 mg, yield 42.0%) as a white solid, which was used directly in the next step without further purification. LC/MS (ESI) m/z: 485 (M+H).

### Step 7: (S)-N-((1H-pyrrolo[3,2-c]pyridin-2-yl)methyl)-7-((5-(3,4-dichlorophenoxy) furan-2-carbonyl)glycyl)-1,4-dioxa-7-azaspiro[4.4]nonane-8-carboxamide (Compound 287)

To a mixture of (S)-7-((5-(3,4-dichlorophenoxy)furan-2-carbonyl)glycyl)-1,4-dioxa-7-azaspiro[4.4]nonane-8-carboxylic acid (26 mg, 0.05 mmol) and (1H-pyrrolo[3,2-c]pyridin-2-yl)methanamine (8 mg, 0.05 mmol) in DMF (2 mL) was added DIPEA (32 mg, 0.25 mmol) and HATU (23 mg, 0.06 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=15:1) to give Compound 287 (1.2 mg, yield 3.9%) as a yellow solid. ¹H NMR (400 MHz, CD₃OD): δ 8.68 (d, J=11.1 Hz, 1H), 8.04 (d, J=5.9 Hz, 1H), 7.54 (d, J=8.9 Hz, 1H), 7.38-7.30 (m, 2H), 7.15-7.05 (m, 2H), 6.54 (s, 1H), 5.86 (d, J=3.6 Hz, 1H), 4.59-4.55 (m, 3H), 4.59-4.55 (m, 3H), 4.14 (s, 1H), 3.99 (d, J=2.9 Hz, 4H), 3.76 (s, 1H), 2.46-2.39 (m, 1H), 2.45-2.39 (m, 1H), 2.29-2.21 (m, 1H), 2.28-2.18 (m, 1H), 1.36 (d, J=6.6 Hz, 1H), 1.29 (d, J=5.7 Hz, 1H). LC/MS (ESI) m/z: 614 (M+H)⁺. RT (Method A): 1.48 min.

### Scheme 81. Synthesis of (2S)-N-((R)-1-(1H-pyrrolo[3,2-c]pyridin-2-yl)ethyl)-4-((4-aminobutoxy) methyl)-1-((phenoxathiine-3-carbonyl)glycyl)pyrrolidine-2-carboxamide (Compound 288)

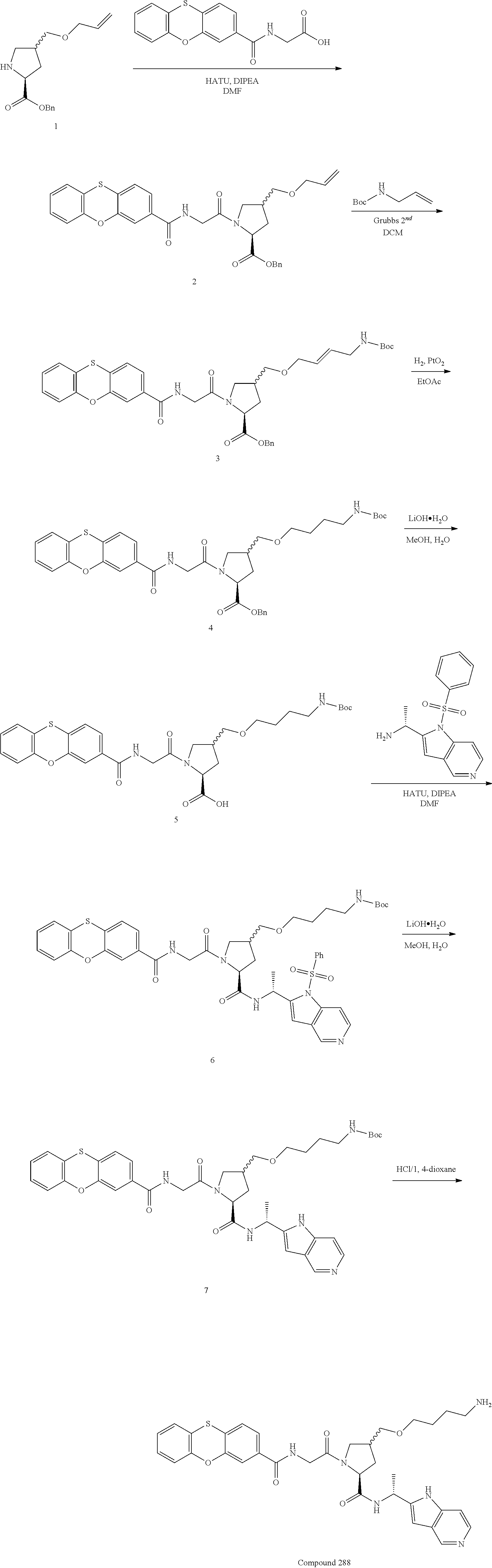

### Step 1: Benzyl (2S)-4-((allyloxy)methyl)-1-((phenoxathiine-3-carbonyl)glycyl) pyrrolidine-2-carboxylate (2)

To a mixture of (phenoxathiine-3-carbonyl)glycine (728 mg, 2.42 mmol) and benzyl (2S)-4-((allyloxy)methyl)pyrrolidine-2-carboxylate (800 mg, 2.91 mmol) in DMF (8 mL) was added DIPEA (1.56 g, 12.1 mmol) and HATU (1.38 g, 3.63 mmol) under N₂ atmosphere and the reaction mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography on silica gel (0-50% EtOAc in PE) to give the title compound (600 mg, yield 44.4%) colorless oil. LC/MS (ESI) m/z: 559 (M+H)⁺.

### Step 2: Benzyl (2S)-4-((((E)-4-((tert-butoxycarbonyl)amino)but-2-en-1-yl)oxy)methyl)-1-((phenoxathine-3-carbonyl)glycyl)pyrrolidine-2-carboxylate (3)

To a mixture of benzyl (2S)-4-((allyloxy)methyl)-1-((phenoxathiine-3-carbonyl)glycyl) pyrrolidine-2-carboxylate (200 mg, 0.36 mmol) and tert-butyl allylcarbamate (113 mg, 0.72 mmol) in dry DCM (200 mL) was added Grubbs 2^{nd} (152 mg, 0.18 mmol) under N₂ atmosphere, the reaction mixture was degassed under N₂ atmosphere for three times and stirred at 30° C. overnight. The mixture was concentrated under reduced pressure to dryness. The residue was purified by flash chromatography (silica gel, 0-50% EtOAc in PE) to give the title compound (70 mg, yield 28.3%) as a brown oil. LC/MS (ESI) m/z: 688 (M+H)⁺.

### Step 3: Benzyl (2S)-4-((4-((tert-butoxycarbonyl))amino)butoxy)methy)-1-((phenoxathiine-3-carbonyl)glycyl)pyrrolidine-2-carboxylate (4)

To a solution of benzyl (2S)-4-((((E)-4-((tert-butoxycarbonyl)amino)but-2-en-1-yl)oxy)methyl)-1-((phenoxathiine-3-carbonyl)glycyl)pyrrolidine-2-carboxylate (70 mg, 0.10 mmol) in EtOAc (2 mL) was added PtO₂ (22 mg, 0.10 mmol). The mixture was degassed under N₂ atmosphere for three times and stirred under a H₂ balloon at room temperature for 3 hours. The mixture was filtered, and the filtrate was concentrated to dryness to give the title compound (35 mg, yield 99%) as a brown oil, which was used directly in the next step without further purification. LC/MS (ESI) m/z: 690 (M+H)⁺.

### Step 4: (2S)-4-((4-((Tert-butoxycarbonyl)amino)butoxy)methyl)-1-((phenoxathiine-3-carbonyl)glycyl)pyrrolidine-2-carboxylic acid (5)

To a solution of benzyl (2S)-4-((4-((tert-butoxycarbonyl)amino)butoxy)methyl)-1-((phenoxathiine-3-carbonyl)glycyl)pyrrolidine-2-carboxylate (35 mg, 0.06 mmol) in MeOH/H₂O (1 mL, 4/1) was added LiOH-1H₂O (5 mg, 0.12 mmol) and the mixture was stirred at room temperature for 2 hours. The mixture was acidified with 1N aq. HCl to pH˜3 and extracted with EtOAc twice. The combined organic layers were washed with brine, dried over anhydrous Na₂SO₄, filtered, and concentrated under reduced pressure to dryness to give the title compound (27 mg, yield 75.0%) as a white solid, which was used directly in the next step without further purification. LC/MS (ESI) m/z: 600 (M+H)⁺.

### Step 5: Tert-butyl (4-(((5S)-1-((phenoxathiine-3-carbonyl)glycyl)-5-(((R)-1-(1-(phenylsulfonyl)-1H-pyrrolo[3,2-c]pyridin-2-yl)ethyl)carbamoyl)pyrrolidin-3-yl)methoxy)butyl)carbamate (6)

To a mixture of (2S)-4-((4-((tert-butoxycarbonyl)amino)butoxy)methyl)-1-((phenoxathiine-3-carbonyl)glycyl)pyrrolidine-2-carboxylic acid (27 mg, 0.045 mmol) and (R)-1-(1-(phenylsulfonyl)-1H-pyrrolo[3,2-c]pyridin-2-yl)ethan-1-amine hydrochloride (25 mg, 0.068 mmol) in DMF (1 mL) was added DIPEA (29 mg, 0.23 mmol) and H ATU (22 mg, 0.059 mmol) under N₂ atmosphere and the reaction mixture was stirred at room temperature for 2 hours. The mixture was concentrated under reduced pressure to dryness. The residue was purified by prep-TLC (DCM:MeOH=10:1) to give the title compound (20 mg, yield 50.4%). LC/MS (ESI) m/z: 883 (M+H)⁺.

### Step 6: Tert-butyl (4-(((5S)-5-(((R)-1-(1H-pyrrolo[3,2-c]pyridin-2-yl)ethyl)carbamoyl)-1-((phenoxathiine-3-carbonyl)glycyl)pyrrolidin-3-yl)methoxy)butyl)carbamate (7)

To a solution of tert-butyl (4-(((5S)-1-((phenoxathiine-3-carbonyl)glycyl)-5-(((R)-1-(1-(phenylsulfonyl)-1H-pyrrolo[3,2-c]pyridin-2-yl)ethyl)carbamoyl)pyrrolidin-3-yl) methoxy)butyl)carbamate (20 mg, 0.022 mmol) in MeOH/H₂O (1 mL, 4/1) was added LiOH H₂O (4 mg, 0.088 mmol) and the mixture was stirred at room temperature for 2 hours. The mixture was concentrated under reduced pressure to dryness. The residue was purified by prep-TLC (DCM:MeOH=7:1) to give the title compound (10 mg, yield 62.5%) as a white solid. LC/MS (ESI) m/z: 743 (M+H)⁺.

### Step 7: (2S)-N-((R)-1-(1H-pyrrolo[3,2-c]pyridin-2-yl)ethyl)-4-((4-aminobutoxy) methyl)-1-((phenoxathiine-3-carbonyl)glycyl)pyrrolidine-2-carboxamide (Compound 288)

A solution of tert-butyl (4-(((5S)-5-(((R)-1-(1H-pyrrolo[3,2-c]pyridin-2-yl)ethyl) carbamoyl)-1-((phenoxathiine-3-carbonyl)glycyl)pyrrolidin-3-yl)methoxy)butyl) carbamate (10 mg, 0.013 mmol) in HCl/1,4-dioxane (1 mL, 4M) was stirred under N₂ atmosphere at room temperature for 2 hours. The reaction mixture was concentrated under reduced pressure to dryness. The residue was purified by prep-HPLC to give Compound 288 (1.3 mg, yield 15.7%) as a yellow solid. ¹H NMR (400 MHz, CD₃OD): δ 7.57 (s, 1H), 7.51-7.47 (m, 1H), 7.40 (d, J=8.9 Hz, 2H), 7.28 (d, J=7.8 Hz, 1H), 7.23-7.17 (m, 2H), 7.15 (d, J=6.1 Hz, 1H), 7.08 (d, J=7.5 Hz, 1H), 7.04 (d, J=7.9 Hz. 1H), 5.00 (s, 2H), 4.83 (s, 1H), 4.42 (s, 2H), 4.32 (d, J=13.1 Hz, 2H), 3.40-3.36 (m, 1H), 2.45-2.35 (m, 1H), 2.20-2.13 (m, 1H), 1.30 (s, 3H), 1.18-1.14 (m, 1H), 0.81-0.75 (m, 1H). LC/MS (ESI) m/z: 643 (M+H)⁺. RT (Method A): 1.05 min.

### Scheme 81. Synthesis of (1S,3S,5S)-N-((1-hydroxy-1,3-dihydrobenzo[c][1,2]oxaborol-6-yl)methyl)-5-methyl-2-((phenoxathiine-3-carbonyl)glycyl)-2-azabicyclo[3.1.0]hexane-3-carboxamide (Compound 290)

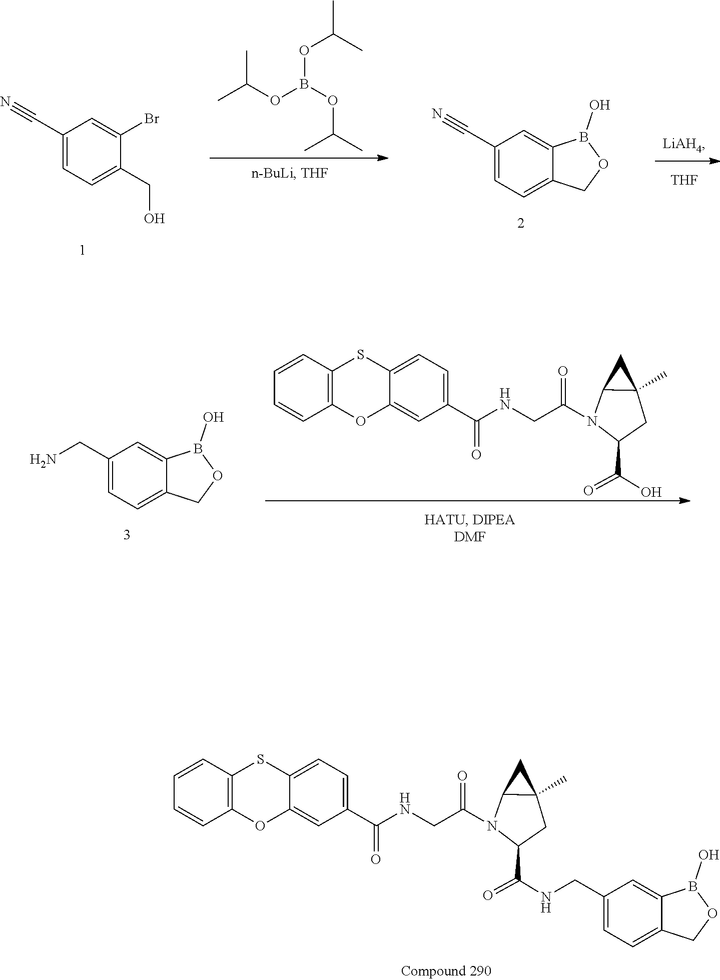

### Step 1: 1-Hydroxy-1,3-dihydrobenzo[c][1,2]oxaborole-6-carbonitrile (2)

To a solution of 3-bromo-4-(hydroxymethyl)benzonitrile (1.0 g, 4.72 mmol) in THE (20 mL) was added triisopropyl borate (1.8 g, 9.4 mmol) at −78° C. The reaction mixture was stirred at −78° C. for 0.5 hour under N₂ atmosphere. Then the reaction mixture was added n-BuLi (5.5 mL, 4.8 mmol, 4 M) at −78° C. and the mixture was stirred at 25° C. overnight. The mixture was quenched with 1 N HCl and diluted with EtOAc, washed with brine, dried over anhydrous Na₂SO₄, filtered, and concentrated under reduced pressure to dryness. The residue was purified by flash chromatography (silica gel, 0-20% EtOAc in PE) to give the title compound (400 mg, yield 53.3%) as a yellow solid.

### Step 2: 6-(Aminomethyl)benzo[c][1,22]oxaborol-1(3H)-ol (3)

To a solution of 1-hydroxy-1,3-dihydrobenzo[c][1,2]oxaborole-6-carbonitrile (150 mg, 0.94 mmol) in THF (3 mL) was added LiAlH₄ (1.88 mL, 1.88 mmol, 1.0 M) at 0° C. The mixture was stirred at 25° C. for 2 hours under N₂ atmosphere. The mixture was quenched with ice-water and MeOH. The suspension was filtrated, and the filtrate was concentrated under reduced pressure to dryness to give the title compound (90 mg, yield 58.8%) as a white solid, which was used directly in the next step without further purification. LC/MS (ESI) m/z: 164 (M+H)⁺.

### Step 3: (1S,3S,5S)-N-((1-hydroxy-1,3-dihydrobenzo[c][1,2]oxaborol-6-yl)methyl)-5-methyl-2-((phenoxathine-3-carbonyl)glycyl)-2-azabicyclo[3.1.0]hexane-3-carboxamide (Compound 290)

To a mixture of (1S,3S,5S)-5-methyl-2-((phenoxathiine-3-carbonyl)glycyl)-2-azabicyclo [3.1.0]hexane-3-carboxylic acid (30 mg, 0.07 mmol) and 6-(aminomethyl) benzo[c][1,2]oxaborol-1(3H)-ol (35 mg, 0.21 mmol) in DMF (2 mL) was added DIPEA (55 mg, 0.42 mmol) and HATU (32 mg, 0.08 mmol)under N₂ atmosphere and the reaction mixture was stirred at 25° C. for 2 hours. The mixture was diluted with EtOAc, washed with brine, dried over anhydrous Na₂SO₄, filtered, and concentrated under reduced pressure to dryness. The residue was purified by prep-TLC (DCM:MeOH=10:1) and further purified by prep-HPLC to give Compound 290 (3 mg, yield 7.5%) as a white solid. ¹H NMR (400 MHz, CD₃OD): δ 7.57 (s, 1H), 7.51-7.47 (m, 1H), 7.40 (d, J=8.9 Hz, 2H), 7.28 (d, J=7.8 Hz, 1H), 7.23-7.17 (m, 2H), 7.15 (d, J=6.1 Hz, 1H), 7.08 (d, J=7.5 Hz, 1H), 7.04 (d, J=7.9 Hz, 1H), 5.00 (s, 2H), 4.83 (s, 1H), 4.42 (s, 2H), 4.32 (d, J=13.1 Hz, 2H), 3.38 (d, J=6.0 Hz, 1H), 2.41 (m, 1H), 2.20-2.13 (m, 1H), 1.30 (s, 3H), 1.16 (d, J=3.2 Hz, 1H), 0.79 (m, 1H). LC/MS (ESI) m/z: 570 (M+H)⁺. RT (Method A): 2.13 min.

Compound 279 was prepared based on Scheme 82:

| Cmpd # | Reactant A | Reactant B | Characterization Data |
|---|---|---|---|
| 279 | 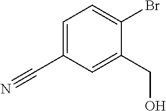 [SMILES: N#Cc1ccc(Br)c(CO)c1] | 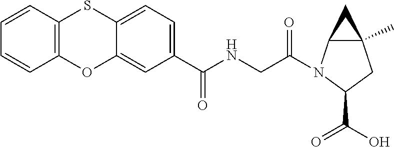 | LC/MS (ESI) m/z: 570 (M + H)⁺. RT (Method A): 2.50 min. |

### Scheme 83. Synthesis of (2-(((1S,3S,5S)-5-methyl-2-((phenoxathiine-3-carbonyl) glycyl)-2-azabicyclo[3.1.0]hexane-3-carboxamido)methyl)-1H-indol-5-yl)boronic acid (Compound 291)

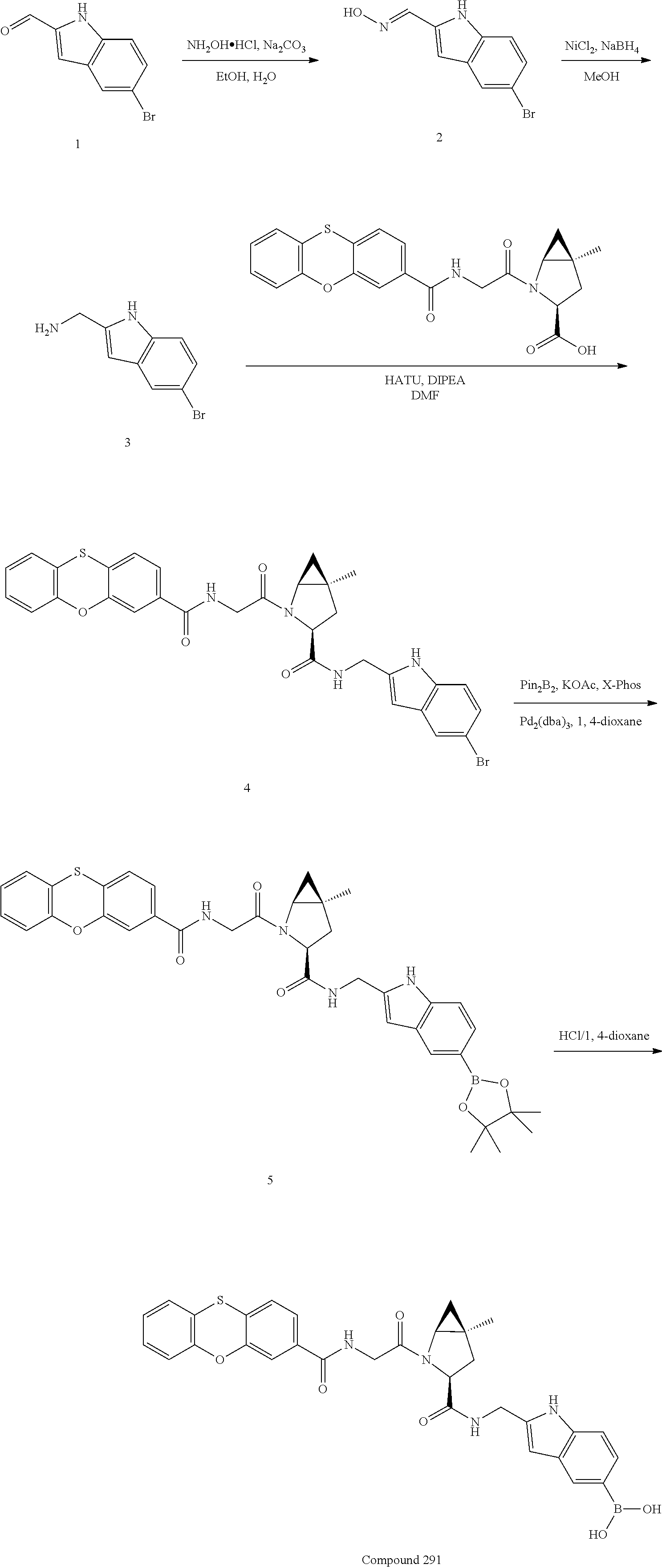

### Step 1: (E)-5-Bromo-1H-indole-2-carbaldehyde oxime (2)

To a mixture of 5-bromo-1H-indole-2-carbaldehyde (250 mg, 1.12 mmol) and NH₂OH·HCl (155 mg, 2.23 mmol) in EtOH (4 mL) and water (1.6 ml) was added Na₂CO₃ (178 mg, 1.67 mmol) under N₂ atmosphere at 0° C. and the mixture was stirred at 60° C. for 1 hour. The mixture was quenched with saturated aq. NH₄Cl solution and extracted with EtOAc twice. The combined organic layers were washed with brine, dried over anhydrous Na₂SO₄, filtered, and concentrated under reduced pressure to dryness. The residue was purified by flash chromatography (silica gel, 0-40% EtOAc in PE) to give the title compound (250 mg, yield 94.3%) as a colorless oil. LC/MS (ESI) (m/z): 239 (M+H)⁺.

### Step 2: (5-Bromo-1H-indol-2-yl)methanamine (3)

To a solution of (E)-5-bromo-1H-indole-2-carbaldehyde oxime (250 mug, 1.05 mmol) in MeOH (3 mL) was added NiCl₂ (144 mg, 1.12 mmol) and NaBH₄ (254 mg, 6.69 mmol) at 0° C. and the mixture was stirred at room temperature for 1 hour. The mixture was quenched with ice-water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% MeOH in DCM) to give the title compound (205 mg, yield 87.6%) as a white solid. LC/MS (ESI) m/z: 225 (M+H)⁺.

### Step 3: (1S,3S,5S)-N-((5-bromo-1H-indol-2-yl)methyl)-5-methyl-2-((phenoxathiine-3-carbonyl)glycyl)-2-azabicyclo[3.1.0]hexane-3-carboxamide (4)

To a mixture of (1S,3S,5S)-5-methyl-2-((phenoxathiine-3-carbonyl)glycyl)-2-azabicyclo[3.1.0]hexane-3-carboxylic acid (57 mg, 0.13 mmol) and (5-bromo-1H-indol-2-yl)methanamine (60 mg, 0.27 mmol) in DMF (2 mL) was added DIPEA (104 mg, 0.81 mmol) and HATU (66 mg, 0.17 mmol) at 0° C. under N₂ atmosphere and the mixture was stirred at room temperature for 1 hour. The mixture was diluted with EtOAc, washed with brine, dried over anhydrous Na₂SO₄, filtered, and concentrated under reduced pressure to dryness. The residue was purified by flash chromatography (silica gel, 0-40% EtOAc in PE) to give the title compound (80 mg, yield 95.2%) as a white solid. LC/MS (ESI) m/z: 631 (M+H)⁺.

### Step 4: (1S,3S,5S)-5-methyl-2-((phenoxathiine-3-carbonyl)glycyl)-N-((5-(4,4,5,5-tetramethyl-1,3,2-dioxaborolan-2-yl)-1H-indol-2-yl)methyl)-2-azabicyclo[3.1.0]hexane-3-carboxamide (5)

To a mixture of (1S,3S,5S)-N-((5-bromo-1H-indol-2-yl)methyl)-5-methyl-2-((phenoxathiine-3-carbonyl)glycyl)-2-azabicyclo[3.1.0]hexane-3-carboxamide (80 mg, 0.13 mmol) and Pin₂B₂ (129 mg, 0.51 mmol) in 1,4-dioxane (3 mL) was added X-phos (19 mg, 0.04 mmol) and Pd₂(dba)₃ (18 mg, 0.02 mmol) and KOAc (50 mg, 0.51 mmol) at 25° C. under N₂ atmosphere and the mixture was stirred at 80° C. for 10 hours. The mixture was added water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% MeOH in DCM) to give the title compound (80 mg, yield 93.0%) as a white solid. LC/MS (ESI) m/z: 679 (M+H)⁺.

### Step 5: (2-(((1S,3S,5S)-5-methyl-2-((phenoxathiine-3-carbonyl)glycyl)-2-azabicyclo [3.1.0]hexane-3-carboxamido)melhyl)-1H-indol-5-yl)boronic acid (Compound 291)

To a solution of (1S,3S,5S)-5-methyl-2-((phenoxathiine-3-carbonyl)glycyl)-N-((5-(4,4,5,5-tetramethyl-1,3,2-dioxaborolan-2-yl)-1H-indol-2-yl)methyl)-2-azabicyclo [3.1.0]hexane-3-carboxamide (30 mg, 0.044 mmol) in THF (2 mL) was added 2 N aq. HCl (1 mL) at 25° C. under N₂ atmosphere and the reaction mixture was stirred at room temperature for 3 hours. The mixture was concentrated under reduced pressure to dryness. The residue was purified by prep-HPLC to give Compound 291 (0.75 mg, yield 2.85%) as a white solid. ¹H NMR (400 MHz, CD₃OD): δ 7.48 (s, 1H), 7.45-7.43 (m, 1H), 7.19 (s, 1H), 7.15 (d, J=7.3 Hz, 1H), 7.08 (d, J=7.5 Hz, 1H), 7.02 (d, J=8.1 Hz, 1H), 6.31 (s, 1H), 4.49-4.37 (m, 2H), 4.29 (d, J=16.8 Hz, 1H), 3.37 (s, 1H), 2.46-2.38 (m, 1H), 2.20 (d, J=12.5 Hz, 1H), 0.89 (d, J=7.1 Hz, 2H). LC/MS (ESI) m/z: 597 (M+H)⁺. RT (Method A): 1.93 min.

### Scheme 84. Synthesis of (1S,3S,5S)-N-((R)-1-(3-aminopropanoyl)pyrrolidin-3-yl)-5-methyl-2-((4-phenoxybutanoyl)glycyl)-2-azabicyclo[3.1.0]hexane-3-carboxamide (Compound 60)

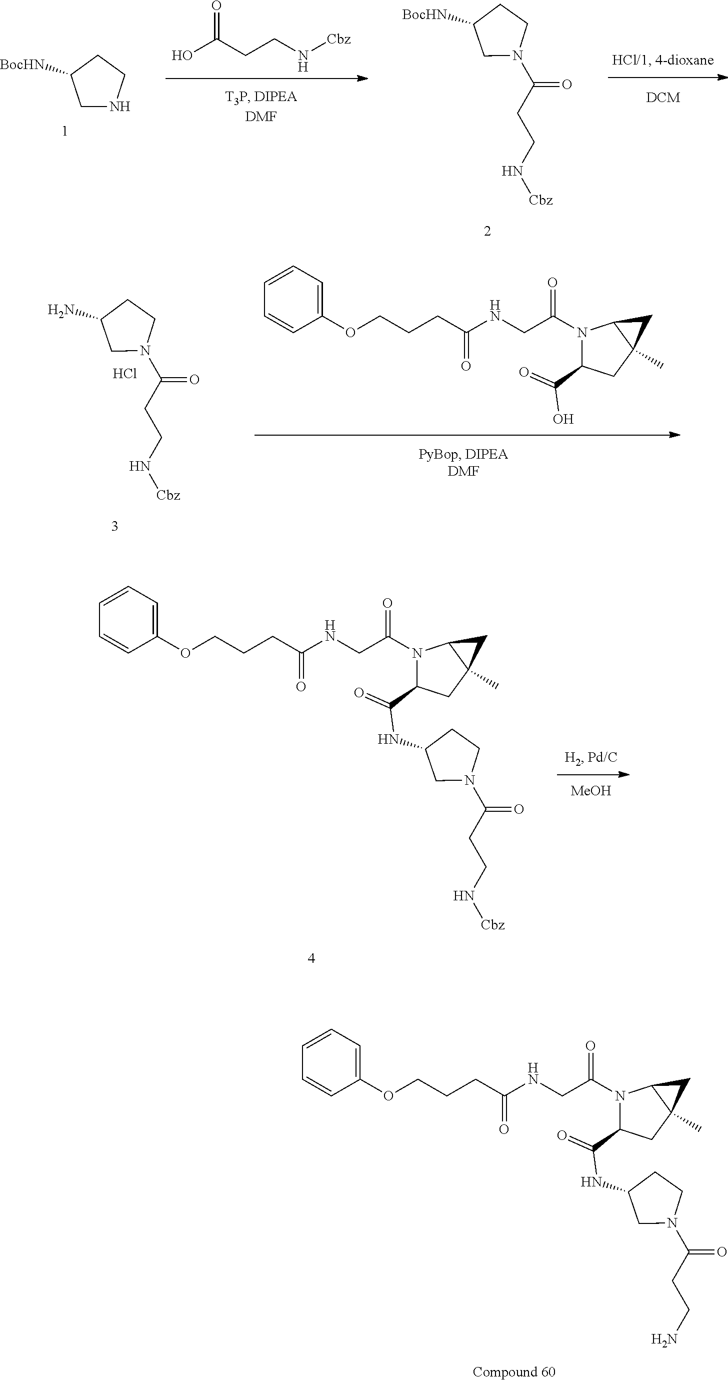

### Step 1: Tert-butyl (R)-(1-(3-(((benzyloxy)carbonyl)amino)propanoyl)pyrrolidin-3-yl)carb amate

To a mixture of tert-butyl (R)-pyrrolidin-3-ylcarbamate (500 mg, 2.69 mmol) and 3-(((benzyloxy)carbonyl)amino)propanoic acid (599 mg, 2.69 mmol) in DMF (6 mL) was added DIPEA (2.09 g, 16.1 mmol) and T₃P (2.56 g, 4.04 mmol, 50% wt. in EtOAc) under N₂ atmosphere at 0° C. and the mixture was stirred at room temperature for 2 hours. The mixture was quenched with saturated aq. NaHCO₃ solution and extracted with EtOAc twice. The combined organic layers were washed with brine, dried over anhydrous Na₂SO₄, filtered, and concentrated under reduced pressure to dryness. The residue was purified by flash chromatography (silica gel, 0-89% EtOAc in PE) to give tert-butyl (R)-(1-(3-(((benzyloxy)carbonyl)amino)propanoyl)pyrrolidin-3-yl)carbamate (850 mg, yield 80.9%) as a white solid. LC/MS (ESI) (m/z): 392 (M+H)⁺.

### Step 2: Benzyl (R)-(3-(3-aminopyrrolidin-1-yl)-3-oxopropyl)carbamate hydrochloride

A solution of tert-butyl (R)-(1-(3-(((benzyloxy)carbonyl)amino)propanoyl)pyrrolidin-3-yl)carbamate (850 mg, 2.17 mmol) in DCM (2 mL) and HCl/1,4-dioxane (8 mL, 4M) was stirred under N₂ atmosphere at room temperature for 2 hours. The reaction mixture was concentrated under reduced pressure to dryness to give benzyl (R)-(3-(3-aminopyrrolidin-1-yl)-3-oxopropyl)carbamate hydrochloride (620 mg, yield 98.0%) as a white solid, which was used directly in the next reaction without further purification. LC/MS (ESI) m/z: 292 (M+H)⁺.

### Step 3: Benzyl (3-((R)-3-((1S,3S,5S)-5-methyl-2-((4-phenoxybutanoyl)glycyl)-2-azabicyclo[3.1.0]hexane-3-carboxamido)pyrrolidin-1-yl-3- oxopropyl)carbamate

To a mixture of (1S,3S,5S)-5-methyl-2-((4-phenoxybutanoyl)glycyl)-2-azabicyclo [3.1.0]hexane-3-carboxylic acid (40 mg, 0.11 mmol) and benzyl (R)-(3-(3-aminopyrrolidin-1-yl)-3-oxopropyl)carbamate hydrochloride (65 mg, 0.22 mmol) in DMF (3 mL) was added DIPEA (86 mg, 0.66 mmol) and PyBOP (69 mg, 0.13 mmol) under N₂ atmosphere and the reaction mixture was stirred at room temperature for 2 hours. The mixture was diluted with EtOAc, washed with saturated aq. NaH CO₃ solution and brine, dried over anhydrous Na₂SO₄, filtered, and concentrated under reduced pressure to dryness. The residue was purified by prep-TLC (DCM:MeOH=10:1) to give benzyl (3-((R)-3-((1S,3S,5S)-5-methyl-2-((4-phenoxybutanoyl)glycyl)-2-azabicyclo [3.1.0]hexane-3-carboxamido)pyrrolidin-1-yl)-3-oxopropyl)carbamate (60 mg, yield 85.3%) as a white solid. LC/MS (ESI) m/z: 634 (M+H)⁺.

### Step 4: (1S,3S,15S)-N((R)-1-(3-aminopropanoyl)pyrrolidin-3yl)-5-methyl-2-((4phenoxybutanoyl)glycyl)-2-azabicyclo[3.1.0]hexane-3-carboxamide (Compound 60)

To a solution of benzyl (3-((R)-3-((1S,3 S,5S)-5-methyl-2-((4-phenoxybutanoyl)glycyl)-2-azabicyclo[3.1.0]hexane-3-carboxamido)pyrrolidin-1-yl)-3-oxopropyl)carbamate (60 mg, 0.09 mmol) in MeOH (2 mL) was added Pd/C (10 mg, 10% wt.), the mixture was degassed under N₂ atmosphere for three times and stirred under a H₂ balloon at 25° C. for 1 hour. The mixture was filtered, and the filtrate was concentrated under reduced pressure to dryness. The residue was purified by prep-HPLC to give Compound 60 (8.0 mg, yield 16.9%) as a white solid. ¹H NMR (400 MHz, CD₃OD): δ 8.41 (s, 1H), 7.25 (t, J=7.9 Hz, 2H), 6.93-6.88 (m, 3H), 4.74-4.68 (m, 1H), 4.28 (d, J=18.2 Hz, 1H), 4.19 (d, J=18.2 Hz, 1H), 4.07 (d, J=16.7 Hz, 1H), 4.02 (t, J=6.3 Hz, 2H), 3.66 (dd, J=10.8, 5.8 Hz, 1H), 3.61-3.55 (m, 1H), 3.51 (dd, J=11.2, 6.4 Hz, 1H), 3.39 (dd, J=14.7, 8.5 Hz, 1H), 3.29 (s, 1H), 3.18 (t, J=5.9 Hz, 2H), 2.68 (t, J=12.5 Hz, 2H), 2.48 (t, J=7.3 Hz, 2H), 2.41-2.33 (m, 1H), 2.19-2.05 (m, 4H), 1.99-1.88 (m, 1H), 1.26 (dd, J=14.4, 3.9 Hz, 4H), 0.80 (dd, J=10.6, 5.2 Hz, 1H). LC/MS (ESI) m/z: 500 (M+H)⁺. RT (Method A): 0.99 min.

### Scheme 85. Synthesis of (1S,3S,5S)-5-methyl-2-((4-phenoxybutanoyl)glycyl)-N-(1-((R)-piperazine-2-carbonyl)azetidin-3-yl)-2-azabicyclo[3.1.0]hexane-3-carboxamide (Compound 296)

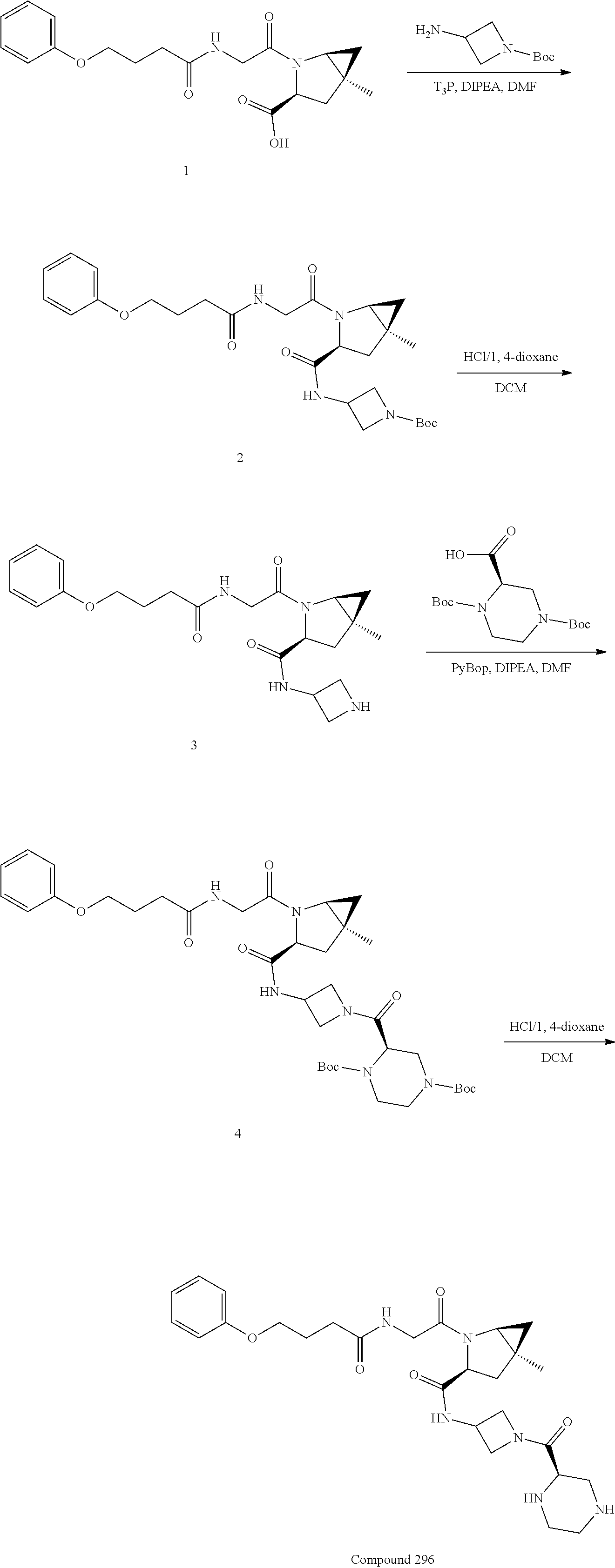

### Step 1: Tert-butyl 3-((1S,3S,5S)-5-methyl-2-((4-phenoxybutanoyl)glycyl)-2-azabicyclo[3.1.0]hexane-3-carboxamido)azetidine-1-carboxylate (1)

To a mixture of (1S,3S,5S)-5-methyl-2-((4-phenoxybutanoyl)glycyl)-2-azabicyclo[3.1.0]hexane-3-carboxylic acid (50 mg, 0.14 mmol) and tert-butyl 3-aminoazetidine-1-carboxylate (26 mg, 0.15 mmol) in DMF (3 mL) was added DIPEA (72 mg, 0.55 mmol) and T₃P (133 mg, 0.20 mmol, 50% wt. in EtOAc) under N₂ atmosphere and the mixture was stirred at room temperature for 2 hours. The mixture was diluted with EtOAc, washed with saturated aq. NaHCO₃ solution and brine, dried over anhydrous Na₂SO₄, filtered, and concentrated under reduced pressure to dryness. The residue was purified by flash chromatography (silica gel, 0-80% EtOAc in PE) to give tert-butyl 3-((1S,3S,5S)-5-methyl-2-((4-phenoxybutanoyl)glycyl)-2-azabicyclo[3.1.0]hexane-3-carboxamido) azetidine-1-carboxylate (28 mg, yield 39.2%) as a white solid. LC/MS (ESI) m/z: 515 (M+H)⁺.

### Step 2: (I S,3S,5S)-N-(azetidin-3-yl)-5-methyl-2-((4-phenoxybutanoyl)glycyl)-2-azabicyclo[3.1.0]hexane-3-carboxamide (2)

A solution of tert-butyl 3-((1S,3S,5S)-5-methyl-2-((4-phenoxybutanoyl)glycyl)-2-azabicyclo[3.1.0]hexane-3-carboxamido)azetidine-1-carboxylate (28 mg, 0.05 mmol) in HCl/1,4-dioxane (1 mL, 4M) was stirred under N₂ atmosphere at room temperature for 3 hours. The reaction mixture was concentrated to dryness under reduced pressure to give (1S,3S,5S)-N-(azetidin-3-yl)-5-methyl-2-((4-phenoxybutanoyl)glycyl)-2-azabicyclo[3.1.0]hexane-3-carboxamide (22 mg, yield 97.6%) as a white solid, which was used directly in the next step without further purification. LC/MS (ESI) m/z: 415 (M+H)⁺.

### Step 3: Di-tert-butyl (R)-2-(3-((1S,3S,5S)-5-methyl-2-((4-phenoxybutanoyl)glycyl)-2-azabicyclo[3.1.0]hexane-3-carboxamido)azetidine-1-carbonyl)piperazine-1,4-dicarboxylate (3)

To a mixture of (1S,3S,5S)-N-(azetidin-3-yl)-5-methyl-2-((4-phenoxybutanoyl)glycyl)-2-azabicyclo[3.1.0]hexane-3-carboxamide (22 mg, 0.05 mmol) and (R)-1,4-bis(tert-butoxycarbonyl)piperazine-2-carboxylic acid (22.7 mg, 0.07 mmol) in DMF (3 mL) was added DIPEA (27.4 mg, 0.21 mmol) and PyBOP (36 mg, 0.07 mmol) under N₂ atmosphere and the reaction mixture was stirred at room temperature for 1 hour. The mixture was diluted with EtOAc, washed with saturated aq. NaHCO₃ solution and brine, dried over anhydrous Na₂SO₄, filtered, and concentrated under reduced pressure to dryness. The residue was purified by prep-TLC (DCM:MeOH=10:1) to give di-tert-butyl (R)-2-(3-((1S,3S,5S)-5-methyl-2-((4-phenoxybutanoyl)glycyl)-2-azabicyclo[3.1.0]hexane-3-carboxamido)azetidine-1-carbonyl)piperazine-1,4-dicarboxylate (38 mg, yield 98.4%) as a white solid. LC/MS (ESI) m/z: 727 (M+H).

### Step 4: (1S,33S,5S)-5-methyl-2-((4-phenoxybutanoyl)glycyl)-N-(1-((R)-piperazine-2-carbonyl)azetidin-3-yl)-2-azabicyclo[3.1.0]hexane-3-carboxamide (Compound 296)

A solution of di-tert-butyl (R)-2-(3-((1S,3S,5S)-5-methyl-2-((4-phenoxybutanoyl) glycyl)-2-azabicyclo[3.1.0]hexane-3-carboxamido)azetidine-1-carbonyl)piperazine-1,4-dicarboxylate (38 mg, 0.05 mmol) in HCl/1,4-dioxane (1.5 mL, 4M) was stirred under N₂ atmosphere at room temperature for 1 hour. The mixture was concentrated under reduced pressure to dryness and the residue was purified by prep-HPLC to give Compound 296 (3.6 mg, yield 13.1%) as a yellow solid. ¹H NMR (400 MHz, CD₃OD): δ 8.40 (s, 2H), 7.27-7.22 (m, 2H), 6.92-6.88 (m, 3H), 4.77-4.73 (m, 1H), 4.54 (ddd, J=19.0, 15.3, 8.0 Hz, 2H), 4.28-4.19 (m, 2H), 4.18-4.09 (m, 2H), 4.01 (t, J=6.2 Hz, 2H), 3.98-3.91 (m, 1H), 3.75-3.71 (m, 1H), 3.26-3.21 (m, 1H), 3.17-2.95 (m, 6H), 2.48 (td, J=7.5, 2.6 Hz, 2H), 2.41-2.34 (m, 1H), 2.11 (ddd, J=13.5, 8.2, 4.4 Hz, 3H), 1.28 (s, 3H), 1.18-1.10 (m, 1H), 0.84-0.78 (m, III). LC/MS (ESI) m/z: 527 (M+H)⁺. RT (Method A): 0.82 min.

### Scheme 86. Synthesis of (2S,4R)-4-(difluoromethoxy)-N-(1H-pyrrolo[3,2-c]pyridin-2-ylmethyl)-1-[2-[[3-(3-thienyloxy)benzoyl]amino]acetyl]pyrrolidine-2-carboxamide (Compound 303)

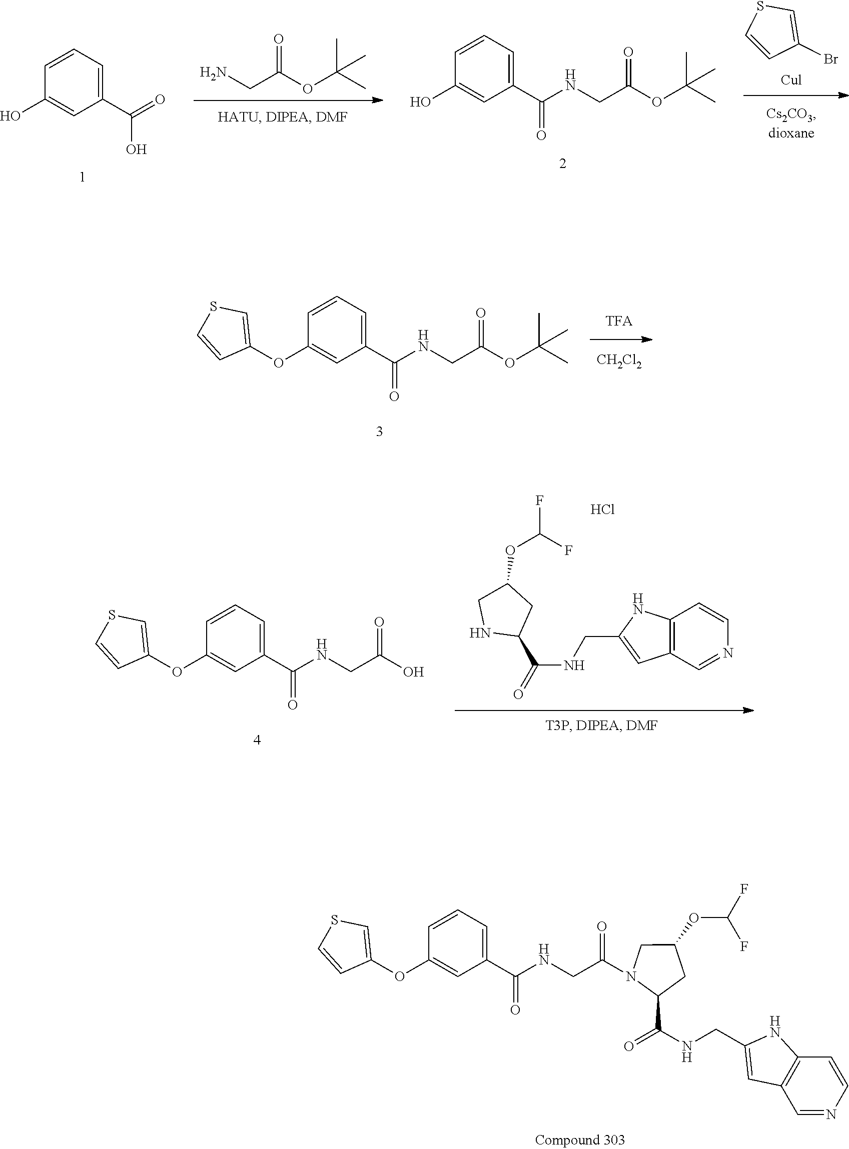

### Step 1: tert-butyl 2-[(3-hydroxybenzoyl)amino]acetate (2)

To a solution of 3-hydroxybenzoic acid (1.000 g, 7.240 mmol, 1.0 equiv.) in DMF (5 mL) was added glycine tert-butyl ester hydrochloride (1.578 g, 9.413 mmol, 1.2 equiv.), HATU (4.129 g, 10.86 mmol, 1.3 equiv.) and DIPEA (0.406 mL, 2.33 mmol, 3.0 equiv.). The reaction was stirred at room temperature for 30 minutes. Water (5 mL) and EtOAc (5 mL) were added, and the two layers separated. The aqueous layer was extracted twice with EtOAc, washed with brine, dried over Na₂SO₄, and concentrated. Purification by Combi Flash, 12 g column, solvent A=CH₂Cl₂, solvent B=MeOH, 100% A to 5% B gave tert-butyl 2-[(3-hydroxybenzoyl)amino]acetate (1.535 g, 6.108 mmol, 84.37% Yield) as a clear oil. LC/MS (ESI) m/z: 252 (M+H)⁺.

### Step 2: tert-butyl 2-[[3-(3-thienyloxy)benzoyl]amino]acetate (3)

A mixture of tert-butyl 2-[(3-hydroxybenzoyl)amino]acetate (55.00 mg, 0.2189 mmol, 1.0 equiv.), 3-bromothiophene (71.37 mg, 0.04377 mmol, 2 equiv.), N,N-dimethylglycine hydrochloride (6.110 mg, 0.04377 mmol, 0.2 equiv.), CuI (4.168 mg, 0.02189 mmol, 0.1 equiv.), and Cs₂CO₃ (0.2860 g, 0.877 mmol, 4 equiv.) in 1,4-dioxane (3 mL) was stirred in a microwave reactor at 110° C. for 12 h. The reaction was cooled to room temperature, diluted with water, extracted with EtOAc, washed with brine, dried over Na₂SO₄ and concentrated. Purification by combi-Flash; 12 g column, solvent A=hexanes, solvent B=EtOAc. 100% A to 15% B gave tert-butyl 2-[[3-(3-thienyloxy)benzoyl]amino]acetate (8.920 mg, 0.02675 mmol, 12.22% Yield). LC/MS (ESI) m/z: 334 (M+H)⁺.

### Step 2: 2-[[3-(3-thienyloxy)benzoyl]amino]acetic acid (4)

TFA (0.5 mL) was added to a solution of tert-butyl 2-[[3-(3-thienyloxy)benzoyl]amino]acetate (8.920 mg, 0.02675 mmol, 1.0 equiv.) in CH₂Cl₂ (1 mL) at ice-bath temperature. The reaction was stirred at room temperature for 2 hours and then concentrated to dryness to give 2-[[3-(3-thienyloxy)benzoyl]amino]acetic acid (7.000 mg, 0.02524 mmol, 94.35% Yield) as a brown solid, which was used in the next step without further purification. LC/MS (ESI) m/z: 278 (M+H)⁺.

### Step 4: (2S,4R)-4-(difluoromethoxy)-N-(JH-pyrrolo[3,2-c]pyridin-2-ylmethyl)-1-[2-[[3-(3-thienyloxy)benzoyl]amino]acetyl]pyrrolidine-2-carboxamide (Compound 303)

To a solution of 2-[[3-(3-thienyloxy)benzoyl]amino]acetic acid (7.000 mg, 0.02524 mmol, 1.1 equiv.) in DMF (2 mL) was added (2S,4R)-4-(difluoromethoxy)-N-(1H-pyrrolo[3,2-c]pyridin-2-ylmethyl)pyrrolidine-2-carboxamide hydrochloride (8.800 mg, 0.02537 mmol, 1.0 equiv.), T3P (50 mass %) in EtOAc (23.00 μL, 0.0384 mmol, 1.5 equiv.), and DIPEA (13.00 μL, 0.0745 mmol, 3.0 equiv.). The reaction mixture was stirred at room temperature for 30 minutes and then purified by prep-HPLC to give Compound 303 (1.900 mg, 0.003336 mmol, 13.15% Yield). LC/MS (ESI) m/z: 570 (M+H)⁺. ¹H NMR (400 MHz, DMSO-d6): δ 11.15 (s, 1H), 8.73 (d, J=15.7 Hz, 1H), 8.63 (d, J=15.6 Hz, 1H), 8.11 (s, 1H), 8.03 (d, J=5.7 Hz, 1H), 7.96 (d, J=5.7 Hz, 1H), 7.71-7.52 (m, 2H), 7.51-7.34 (m, 1H), 7.24 (dd, J=8.3, 2.9 Hz, 2H), 7.10 (d, J=5.7 Hz, 1H), 7.09-6.99 (m, 1H), 6.84 (dd, J=5.8, 3.8 Hz, 1H), 6.73 (d, J=14.6 Hz, 1H), 6.68-6.60 (m, 1H), 6.60-6.42 (m, 1H), 6.32 (s, 1H), 4.49-4.21 (m, 2H), 4.13 (dd, J=16.8, 5.8 Hz, 1H), 3.95 (dd, J=16.8, 5.7 Hz, 1H), 3.90-3.67 (m, 2H), 2.30-2.14 (m, 2H), 2.09 (dt, J=13.2, 6.5 Hz, 1H). RT (Method A): 1.28 min.

Compounds 366 and 385 were prepared based on Steps 2-4 in Scheme 86:

| Cmpd # | Reactant A | Reactant B | Reactant C | Characterization Data |
|---|---|---|---|---|
| 366 | 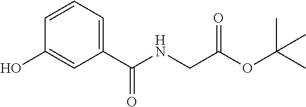 | 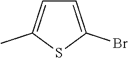 | 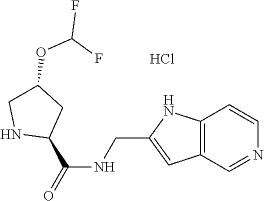 [SMILES: O=C(NCc1cc2cnccc2[nH]1)[C@@H]1C[C@@H](OC(F)F)CN1 ; Cl] | ¹H NMR (400 MHz, DMSO-d6): δ 11.22 (s, IH), 8.79- 8.51 (m, 2H), 8.25 (s, 1H), 8.02 (d, J = 5.6 Hz, 1H), 7.72- 7.50 (m, 1H), 7.37-7.24 (m, 2H), 7.17 (d, J - 5.5 Hz, 1H), 6.79 (d, J = 14.5 Hz, 1H), 6.70-6.55 (m, 1H), 6.56-6.44 (m, 2H), 6.38 (s, 1H), 5.01-4.93 (m, 1H), 4.44 (t, J - 7.1 Hz, 3H), 4.19 (dd, J = 16.8, 5.9 Hz, 1H), 4.02 (dd, J = 16.7, 5.7 Hz, 1H), 3.94-3.86 (m, 1H), 3.85-3.73 (m, 1H), 2.37 (s, 3H), 2.16 (ddd, J = 13.0, 7.5, 5.0 Hz, 1H). LC/MS (ESI) m/z: 584 (M + H)⁺. RT (Method A): 1.49 min. |
| 385 | 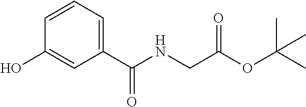 | 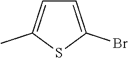 | 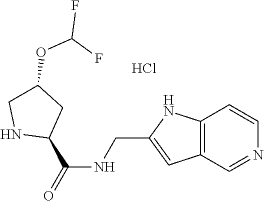 [SMILES: O=C(NCc1cc2cnccc2[nH]1)[C@@H]1C[C@@H](OC(F)F)CN1 ; Cl] | ¹H NMR (400 MHz, DMSO-d6): δ 11.09 (s, 1H), 8.58 (d, J = 5.6 Hz, 2H), 8.23 (s, 1H), 7.91 (d, J = 5.6 Hz, 1H), 7.76 (d, J = 8.0 Hz, IH), 7.68 (s, 1H), 7.64-7.46 (m, 2H), 7.37 (ddt, J = 11.8, 9.3, 2.6 Hz, 1H), 7.19 (td, J = 8.4, 2.7 Hz, 1H), 6.99 (d, J = 5.6 Hz, 1H), 6.32 (s, 1H), 4.63-4.23 (m, 3H), 4.00-3.82 (m, 1H), 3.77 (d, J = 10.8 Hz, 1H), 3.59 (d, J = 10.8 Hz, 1H), 2.43 (s, 3H), 2.28 (d, J = 13.0, 8.5 Hz, 1H), 2.04 (dd, J = 13.0, 7.4 Hz, 1H). LC/MS (ESI) m/z: 584 (M + H)⁺. RT (Method A): 1.47 min. |

Compound 326 can be prepared based on Steps 2-5 in Scheme 86

| Cmpd # | Reactant A | Reactant B | Reactant C |
|---|---|---|---|
| 326 | 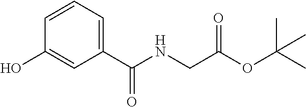 [SMILES: CC(C)(C)OC(=O)CNC(=O)c1cccc(O)c1] | 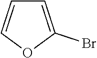 | 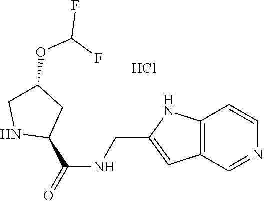 [SMILES: O=C(NCc1cc2cnccc2[nH]1)[C@@H]1C[C@@H](OC(F)F)CN1 ; Cl] |

### Scheme 87. Synthesis of (2S,4R)-N-((1H-pyrrolo[3,2-c]pyridin-2-yl)methyl)-4-(difluoromethoxy)-1-((4-fluoro-3-phenoxybenzoyl)glycyl)pyrrolidine-2-carboxamide (Compound 304)

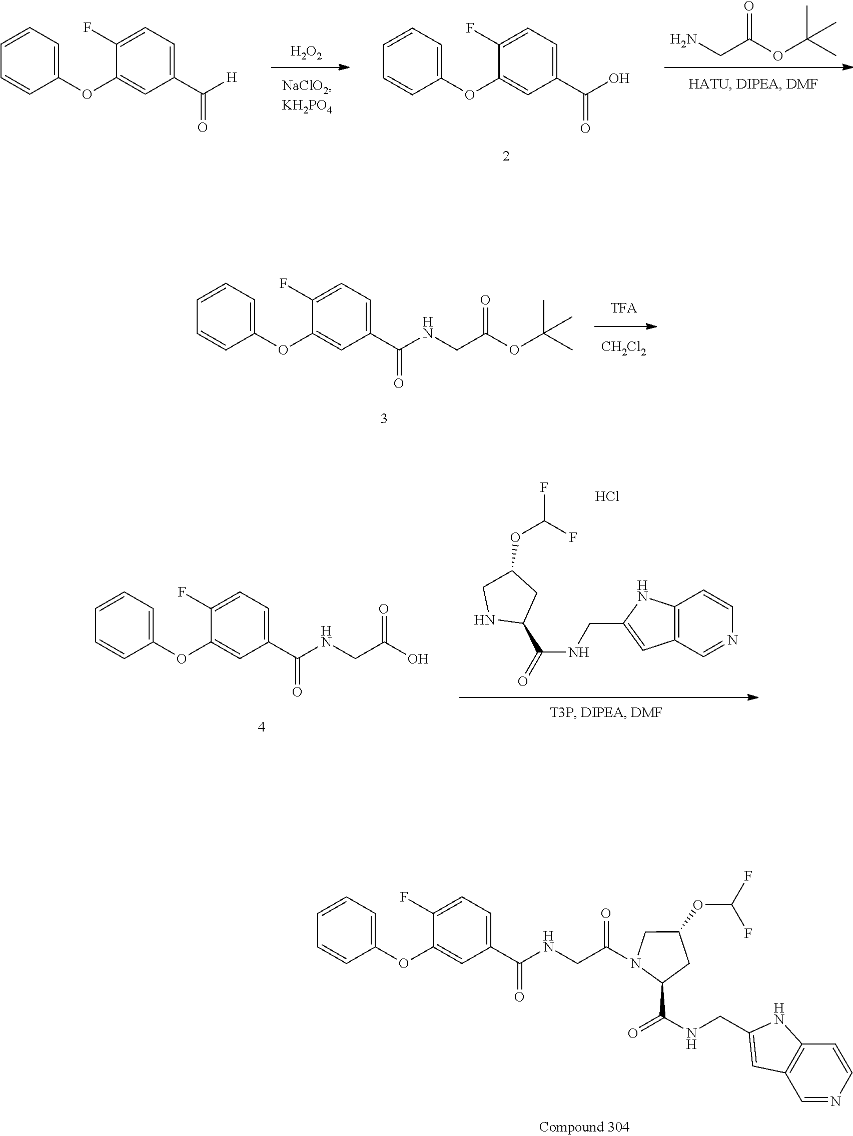

### Step 1: 4-fluoro-3-phenoxybenzoic acid (2)

4-Fluoro-3-phenoxy benzaldehyde (200.0 mg, 0.9251 mmol, 1 equiv.) in THE (2 mL) and H₂O (1 mL) was added hydrogen peroxide (0.1608 mL, 1.850 mmol, 2.0 equiv.) and sodium chlorite (0.1673 g, 1.850 mmol, 2.0 equiv.), and potassium dihydrogen phosphate (0.3777 g, 2.775 mmol, 3.0 equiv.). The reaction mixture was stirred at room temperature overnight and then quenched with saturated sodium thiosulfate. The two layers were separated, and the aqueous layer was extracted with EtOAc. The combined organic extracts were washed with brine, dried over Na₂SO₄, and concentrated to give 4-fluoro-3-phenoxy-benzoic acid (201.0 mg, 0.8656 mmol, 93.57% Yield.).

### Step 2: tert-butyl (4-fluoro-3-phenoxybenzoyl)glycinate (3)

To a solution of 4-fluoro-3-phenoxy-benzoic acid (0.45 g, 1.9 mmol, 1.0 equiv.) in DMF (5 mL) was added glycine tert-butyl ester hydrochloride (0.4223 g, 2.519 mmol, 1.2 equiv.), HATU (1.105 g, 2.906 mmol, 1.3 equiv.) and DIPEA (1.010 mL, 5.79 mmol, 3.0 equiv.). The reaction was stirred at room temperature for 30 minutes. Water (5 mL) and EtOAc (5 mL) were added, and the two layers separated. The aqueous layer was extracted twice with EtOAc washed with brine, dried over Na₂SO₄, and concentrated. Purification by Combi Flash, 12 g column, solvent A=CH₂Cl₂, solvent B=MeOH, 100% A to 5% B gave tert-butyl 2-[(4-fluoro-3-phenoxy-benzoyl)amino]acetate (0.4138 g, 1.198 mmol, 62% Yield) as a white solid. LC/MS (ESI) m/z: 346 (M+H)⁺.

### Step 3: (4-fluoro-3-phenoxybenzoyl)glycine (4)

TFA (0.5 mL) was added to a solution of tert-butyl 2-[(4-fluoro-3-phenoxy-benzoyl)amino]acetate (50.00 mg, 0.1448 mmol, 1.0 equiv.) in CH₂Cl₂ (1 mL) at ice-bath temperature. The reaction was stirred at room temperature for 2 hours and then concentrated to dryness to give 2-[(4-fluoro-3-phenoxy-benzoyl)amino]acetic acid (41.00 mg, 0.1417 mmol, 97.90% Yield) as a brown solid, which was used in the next step without further purification. LC/MS (ESI) m/z: 290 (M+H)⁺.

### Step 4: (2S,4R)-N-((1H-pyrrolo[3,2-c]pyridin-2-yl)methyl)-4-(difluoromethoxy)-1-((4-fluoro-3-phenoxybenzoyl)glycyl)pyrrolidine-2-carboxamide (Compound 304)

To a solution of 2-[(4-fluoro-3-phenoxy-benzoyl)amino]acetic acid (9.176 mg, 0.03172 mmol, 1.1 equiv.) in DMF (2 mL) was added (2S,4R)-4-(difluoromethoxy)-N-(1H-pyrrolo[3,2-c]pyridin-2-ylmethyl)pyrrolidine-2-carboxamide hydrochloride (10.00 mg, 0.02884 mmol, 1.0 equiv.), T3P (50 mass %) in EtOAc (25.90 μL, 0.0433 mmol, 1.5 equiv.), and DIPEA (15.10 μL, 0.0866 mmol, 3.0 equiv.). The reaction mixture was stirred at room temperature for 30 minutes and then purified by prep-HPLC to give Compound 304 (2.500 mg, 0.004299 mmol, 14.91% Yield). LC/MS (ESI) m/z: 582 (M+H)⁺. ¹H NMR (400 MHz, DMSO-d6): δ 11.39 (s, 1H), 8.89 (t, J=5.6 Hz, 1H), 8.80 (t, J=5.8 Hz, 1H), 8.75 (d, J=8.4 Hz, 2H), 8.63 (t, J=5.8 Hz, 1H), 8.13 (s, 1H), 8.08 (d, J=5.8 Hz, 1H), 7.86-7.73 (m, 1H), 7.72-7.62 (m, 1H), 7.53 (dd, J=10.6, 8.6 Hz, 1H), 7.47-7.35 (m, 1H), 7.28 (d, J=5.8 Hz, 1H), 7.16 (q, J=7.0 Hz, 2H), 7.02 (q, J=9.3 Hz, 1H), 6.80 (s, 1H), 6.50 (s, 1H), 4.96 (s, 1H), 4.51 (t, J=6.3 Hz, 1H), 4.47-4.34 (m, 1H), 4.17 (dd, J=16.8, 5.8 Hz, 1H), 4.07-3.96 (m, 1H), 3.94-3.84 (m, 1H), 3.82-3.69 (m, 1H), 3.58 (dd, J=12.6, 4.9 Hz, 1H), 2.42-2.29 (m, 2H), 2.22-2.07 (m, 1H). RT (Method A): 1.42 min.

### Scheme 88. Synthesis of (2S,4R)-4-(difluoromethoxy)-1-[2-[[4-fluoro-3-(3-thienyloxy)benzoyl]amino]acetyl]-N-(1H-pyrrolo[3,2-c]pyridin-2-ylmethyl)pyrrolidine-2-carboxamide (Compound 317)

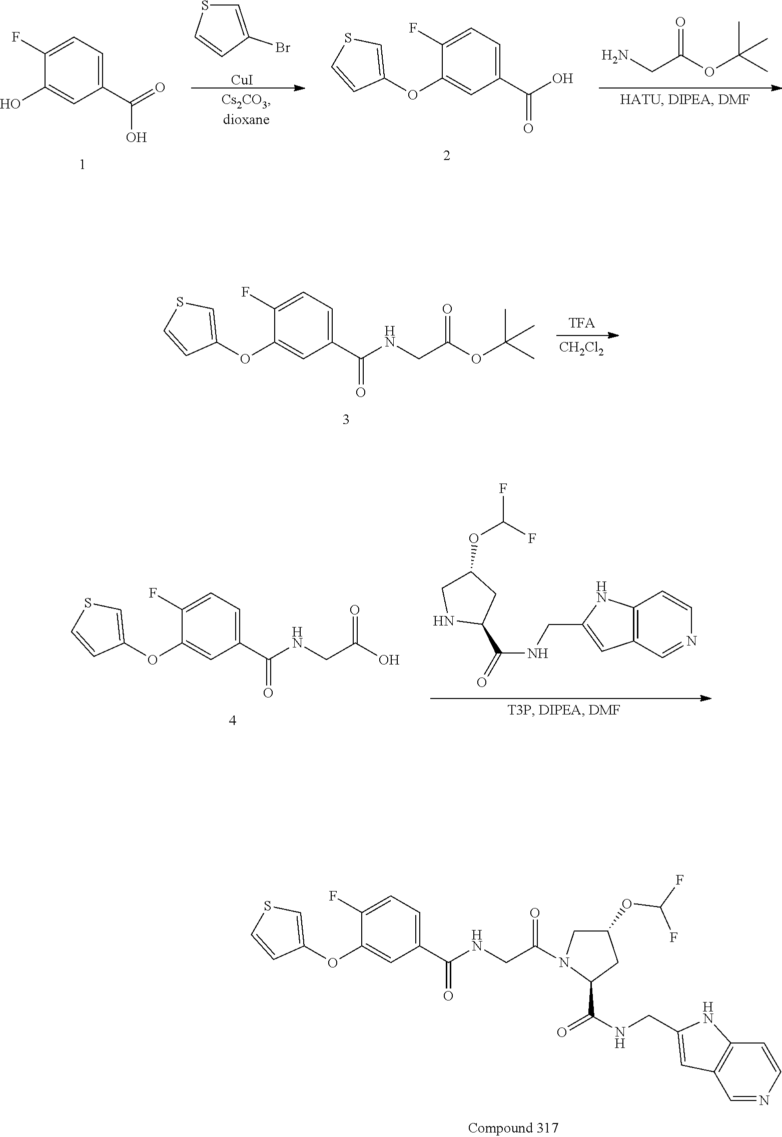

### Step 1: 4-fluoro-3-(3-thienyloxy)benzoic acid (2)

A mixture of 4-fluoro-3-hydroxy-benzoic acid (50.00 mg, 0.3203 mmol, 1.0 equiv.), 3-bromothiophene (0.1044 g, 0.6403 mmol, 2 equiv.), N,N-dimethylglycine hydrochloride (8.941 mg, 0.06406 mmol, 0.2 equiv.), CuI (6.100 mg, 0.03203 mmol, 0.1 equiv.), Cs₂CO₃ (0.418 g, 1.28 mmol, 4 equiv.) in 1,4-dioxane (3 mL) was stirred in a microwave reactor at 110° C. for 12 hours. The reaction was cooled to room temperature, diluted with water and EtOAc. The two layers were separated, the aqueous layer was extracted with EtOAc, washed with brine, dried over Na₂SO₄ and concentrated. Purification by combi-Flash; 4 g column, solvent A=hexanes, solvent B=EtOAc. 100% A to 15% B to give 4-fluoro-3-(3-thienyloxy)benzoic acid (11.34 mg, 0.04761 mmol, 14.86% Yield). LC/MS (ESI) m/z: 239 (M+H)⁻.

### Step 2: 2-[[4-fluoro-3-(3-thienyloxy)benzoyl]amino]acetate (3)

To a solution of 4-fluoro-3-(3-thienyloxy)benzoic acid (11.34 mg, 0.04761 mmol, 1 equiv.) in DMF (2 mL) was added tert-butyl 2-aminoacetate hydrochloride (11.97 mg, 0.07141 mmol, 1.5 equiv.), T3P (50 mass %) in EtOAc (42.80 μL, 0.0715 mmol, 1.3 equiv.) and DIPEA (24.90 μL, 0.143 mmol, 3 equiv.). The reaction was stirred at room temperature for 1 h and the purified by HPLC to give tert-butyl 2-[[4-fluoro-3-(3-thienyloxy)benzoyl]amino]acetate (15.00 mg, 0.04269 mmol, 89.66% Yield). LC/MS (ESI) m/z: 352 (M+H)⁺.

### Step 3: 2-[[4-fluoro-3-(3-thienyloxy)benzoyl]amino]acetic acid (4)

TFA (0.5 mL) was added to a solution of tert-butyl 2-[[4-fluoro-3-(3-thienyloxy)benzoyl]amino]acetate (11.34 mg, 0.03227 mmol, 1.0 equiv.) and in CH₂Cl₂ (1 mL) ice-bath temperature. The reaction was stirred at room temperature for 2 hours and then concentrated to give 2-[[4-fluoro-3-(3-thienyloxy)benzoyl]amino]acetic acid (9.500 mg, 0.03217 mmol, 99.69% Yield). LC/MS (ESI) m/z: 296 (M+H)⁺.

### Step 4: (2S,4R)-4-(difluoromethoxy)-1-[2-[[4-fluoro-3-(3-thienyloxy)benzoyl]amino]acetyl]-N-(H-pyrrolo[3,2-c]pyridin-2-ylmethyl)pyrrolidine-2-carboxamide (Compound 317)

To a solution of 2-[[4-fluoro-3-(3-thienyloxy)benzoyl]amino]acetic acid (9.366 mug, 0.03172 mmol, 1.1 equiv.) in DMF (2 mL) was added (2S,4R)-4-(difluoromethoxy)-N-(1H-pyrrolo[3,2-c]pyridin-2-ylmethyl)pyrrolidine-2-carboxamide hydrochloride (10.00 mg, 0.02884 mmol, 1.0 equiv.), T3P (50 mass %) in EtOAc (25.90 μL, 0.0433 mmol, 1.5 equiv.), and DIPEA (15.10 μL, 0.0866 mmol, 3.0 equiv.). The reaction mixture was stirred at room temperature for 30 minutes and then purified by prep-HPLC to give Compound 317 (2.8 mg, 0.0048 mmol, 17% Yield). LC/MS (ESI) m/z: 588 (M+H)⁺. ¹H NMR (400 MHz, DMSO-d6): δ 11.23 (s, 1H), 8.92 (s, 1H), 8.78 (d, J=17.9 Hz, 1H), 8.75-8.59 (m, 2H), 8.38 (s, 1H), 8.03 (d, J=5.5 Hz, 1H), 7.74 (s, 1H), 7.72- 7.64 (m, 1H), 7.58 (dd, J=5.3, 3.4 Hz, 1H), 7.57-7.45 (m, 1H), 7.18 (d, J=5.6 Hz, 1H), 6.98 (dt, J=10.2, 5.0 Hz, 1H), 6.91 (dd, J=3.3, 1.6 Hz, 1H), 6.80 (s, 1H), 6.37 (s, 1H), 5.31-4.75 (m, 2H), 4.58-4.19 (m, 1H), 3.95-3.63 (m, 2H), 2.43-2.06 (m, 2H). RT (Method A): 1.35 min.

### Scheme 89. Synthesis of (2S,4R)-4-(difluoromethoxy)-1-[2-[[5-(2-pyridyl)thiophene-2-carbonyl]amino]acetyl]-N-(1H-pyrrolo[3,2-c]pyridin-2-ylmethyl)pyrrolidine-2-carboxamide (Compound 381)

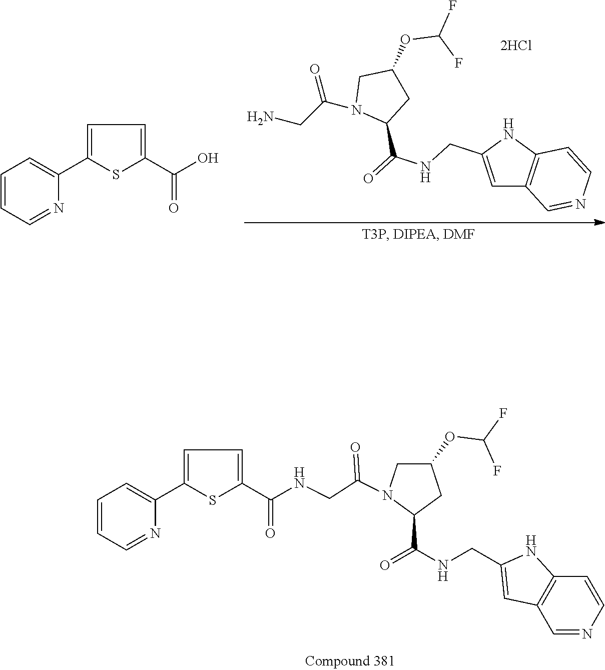 [SMILES: O=C(O)c1ccc(-c2ccccn2)s1 ; NCC(=O)N1C[C@H](OC(F)F)C[C@H]1C(=O)NCc1cc2cnccc2[nH]1 ; O=C(NCC(=O)N1C[C@H](OC(F)F)C[C@H]1C(=O)NCc1cc2cnccc2[nH]1)c1ccc(-c2ccccn2)s1]

To a solution of 5-(2-pyridyl)thiophene-2-carboxylic acid (4.102 mg, 0.01999 mmol, 1.0 equiv.) in DMF (2 mL) was added (2S,4R)-1-(2-aminoacetyl)-4-(difluoromethoxy)-N-(1H-pyrrolo[3,2-c]pyridin-2-ylmethyl)pyrrolidine-2-carboxamidc;dihydrochloride (8.000 mg, 0.01817 mmol, 1.0 equiv.), T3P (50 mass %) in EtOAc (16.30 μL, 0.0272 mmol, 1.5 equiv.), and DIPEA (9.510 μL, 0.0545 mmol, 3.0 equiv.). The reaction mixture was stirred at room temperature for 30 minutes and then purified by prep-HPLC to give Compound 381 (2.461 mg, 0.004437 mmol, 24.42% Yield). LC/MS (ESI) m/z:555 (M+H)⁺. ¹H NMR (400 MHz, DMSO-d6): δ 11.22 (s, 1H), 8.93 (s, 1H), 8.79 (t, J=6.2 Hz, 1H), 8.73-8.64 (m, 1H), 8.64-8.50 (m, 1H), 8.29 (s, 1H), 8.10 (d, J=5.7 Hz, 1H), 8.09-7.94 (m, 1H), 7.94-7.72 (m, 2H), 7.42-7.26 (m, 1H), 7.19 (d, J=5.6 Hz, 1H), 7.00 (s, 1H), 6.82 (s, 1H), 6.63 (s, 1H), 6.39 (s, 1H), 4.98 (s, 1H), 4.68 (d, J=7.1 Hz, 1H), 4.59-4.36 (in, 1H), 4.21 (dd, J=16.9, 5.9 Hz, 1H), 4.04 (dd, J=16.7, 5.7 Hz, 1H), 3.96-3.85 (m, 1H), 3.80 (d, J=10.8 Hz, 1H), 2.33 (d, J=3.8 Hz, 1H), 2.17 (dd, J=13.2, 6.8 Hz, 1H). RT (Method A): 0.93 min.

The following compounds were prepare based on Scheme 381:

| Cmpd | | |
|---|---|---|
| # | Reactant A | Reactant B |
| 377 | 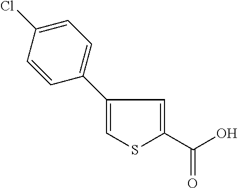 [SMILES: O=C(O)c1cc(-c2ccc(Cl)cc2)cs1] | 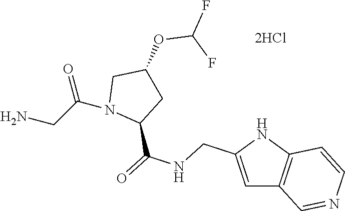 [SMILES: NCC(=O)N1C[C@H](OC(F)F)C[C@H]1C(=O)NCc1cc2cnccc2[nH]1] |
| 378 | 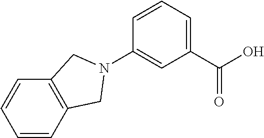 [SMILES: O=C(O)c1cccc(N2Cc3ccccc3C2)c1] | 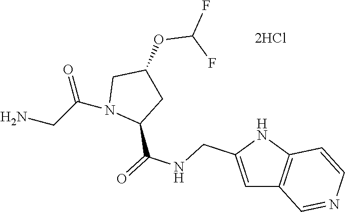 [SMILES: NCC(=O)N1C[C@H](OC(F)F)C[C@H]1C(=O)NCc1cc2cnccc2[nH]1] |
| 379 | 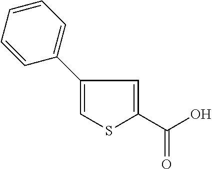 [SMILES: O=C(O)c1cc(-c2ccccc2)cs1] | 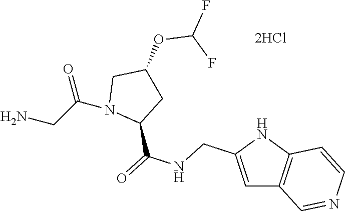 [SMILES: NCC(=O)N1C[C@H](OC(F)F)C[C@H]1C(=O)NCc1cc2cnccc2[nH]1] |
| 380 | 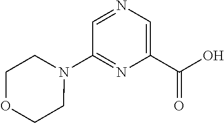 [SMILES: O=C(O)c1cncc(N2CCOCC2)n1] | 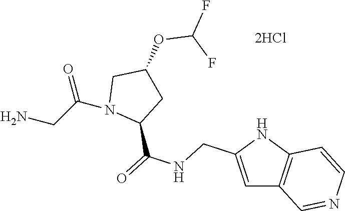 [SMILES: NCC(=O)N1C[C@H](OC(F)F)C[C@H]1C(=O)NCc1cc2cnccc2[nH]1] |
| 382 | 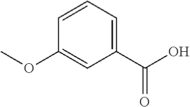 | 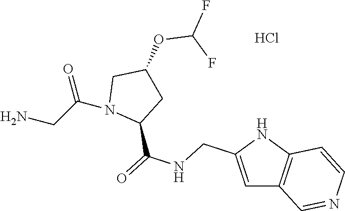 [SMILES: NCC(=O)N1C[C@H](OC(F)F)C[C@H]1C(=O)NCc1cc2cnccc2[nH]1 ; Cl] |
| 384 | 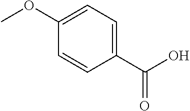 | 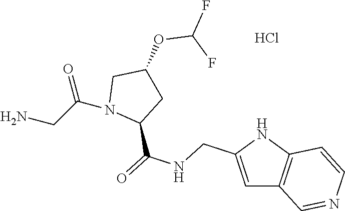 [SMILES: NCC(=O)N1C[C@H](OC(F)F)C[C@H]1C(=O)NCc1cc2cnccc2[nH]1 ; Cl] |

| Cmpd # | Characterization Data |
|---|---|
| 377 | ¹H NMR (400 MHz, DMSO-d6): δ 11.21 (s, 1H), 8.94 (s, 1H), 8.78 (t, J = 6.0 |
| | Hz, 1H), 8.73-8.61 (m, 1H), 8.35 (s, 1H), 8.30 (d, J = 5.2 Hz, 1H), 8.23-8.13 |
| | (m, 1H), 7.96 (d, J = 5.6 Hz, 1H), 7.82-7.67 (m, 1H), 7.59-7.48 (m, 1H), 7.18 |
| | (d, J = 5.7 Hz, 1H), 7.00 (s, 1H), 6.81 (s, 1H), 6.63 (s, 1H), 6.38 (s, 1H), 4.98 (s, |
| | 1H), 4.70 (t, J = 7.4 Hz, 1H), 4.51 (d, J = 6.0 Hz, 1H), 4.49-4.33 (m, 1H), 4.24 |
| | (dd, J = 17.0, 5.9 Hz, 1H), 4.07 (dd, J = 16.9, 5.6 Hz, 1H), 3.90 (dd, J = 11.6, 4.6 |
| | Hz, 1H), 3.85-3.73 (m, 1H), 2.77-2.59 (m, 1H), 2.42-2.27 (m, 1H), 2.16 (dt, |
| | J = 13.2, 6.7 Hz, 1H). LC/MS (ESI) m/z: 589 (M + H)⁺. |
| 378 | ¹H NMR (400 MHz, DMSO-d6): δ 11.22 (s, 1H), 8.73 (s, 1H), 8.73-8.55 (m, |
| | 2H), 8.35 (s, 1H), 8.10 (d, J = 5.6 Hz, 1H), 8.00 (d, J = 5.6 Hz, 1H), 7.48-7.38 |
| | (m, 2H), 7.39-7.28 (m, 2H), 7.27-7.13 (m, 2H), 6.91-6.80 (m, 1H), 6.39 (s, |
| | 1H), 4.99 (s, 1H), 4.66 (d, J = 3.6 Hz, 2H), 4.53 (t, J = 6.1 Hz, 1H), 4.51-4.36 |
| | (m, 3H), 4.21 (dd, J = 16.7, 5.7 Hz, 1H), 4.06 (dd, J = 16.8, 5.8 Hz, 1H), 3.91 |
| | (dd, J = 11.4, 4.6 Hz, 1H), 3.87-3.74 (m, 1H), 3.63 (s, 1H), 2.67 (t, J = 1.9 Hz, |
| | 1H), 2.43-2.31 (m, 1H), 2.17 (dt, J = 13.2, 6.3 Hz, 1H). LC/MS (ESI) m/z: |
| | 589 (M + H)⁺. RT (Method A): 1.52 min. |
| 379 | ¹H NMR (400 MHz, DMSO-d6): δ 11.19 (s, 1H), 8.92 (s, 1H), 8.79 (t, J = 5.8 |
| | Hz, 1H), 8.68 (d, J = 3.1 Hz, 1H), 8.41-8.31 (m, 1H), 8.15-8.06 (m, 1H), 7.96 |
| | (d, J = 5.6 Hz, 1H), 7.78-7.65 (m, 2H), 7.46 (t, J = 7.6 Hz, 1H), 7.34 (t, J = 7.1 |
| | Hz, 1H), 7.18 (d, J = 5.6 Hz, 1H), 6.82 (s, 1H), 6.38 (s, 1H), 4.99 (s, 1H), 4.55- |
| | 4.36 (m, 1H), 4.25 (dd, J = 16.9, 5.9 Hz, 1H), 4.07 (dd, J = 16.9, 5.7 Hz, 1H), |
| | 3.90 (dd, J = 11.5, 4.7 Hz, 1H), 3.87-3.72 (m, 1H), 2.75-2.64 (m, 1H), 2.33 |
| | (d, J = 1.9 Hz, 1H), 2.17 (dt, J = 13.2, 6.2 Hz, 1H). LC/MS (ESI) m/z: 554 |
| | (M + H)⁺. RT (Method A): 1.30 min. |
| 380 | ¹H NMR (400 MHz, DMSO-d6): δ 11.25 (s, 1H), 9.00 (s, 1H), 8.76 (s, 1H), 8.73- |
| | 8.60 (m, 1H), 8.61-8.49 (m, 1H), 8.41 (d, J = 2.5 Hz, 1H), 8.37 (s, 1H), 8.09 |
| | (s, 1H), 8.08-7.97 (m, 1H), 7.30 (s, 1H), 7.20-7.14 (m, 2H), 7.00 (s, 1H), 6.88- |
| | 6.77 (m, 1H), 6.46 (s, 1H), 6.38 (s, 1H), 6.25 (s, 1H), 5.06-4.89 (m, 2H), 4.60- |
| | 4.38 (m, 3H), 4.24-4.07 (m, 2H), 3.88 (d, J = 12.5 Hz, 1H), 3.81-3.71 (m, |
| | 2H), 3.68 (d, J = 4.7 Hz, 2H), 2.76-2.59 (m, 2H). 2.33 (d, J = 6.8 Hz, 1H), 2.18 |
| | (d, J = 7.4 Hz, 1H). LC/MS (ESI) m/z: 559 (M + H)⁺. RT (Method A): 0.60 min. |
| 382 | ¹H-NMR (400 MHz, DMSO-d6): δ 11.33 (s, 1H), 9.17-8.48 (m, 3H), 8.35- |
| | 7.94 (m, 1H), 7.77-6.33 (m, 4H), 5.37-4.36 (m, 2H), 4.15 (m, 2H), 3.98- |
| | 3.76 (m, 4H), 2.28 (s, 3H). LC/MS (ESI) m/z: 502 (M + H)⁺. RT (Method A): |
| | 0.78 min. |
| 384 | ¹H-NMR (400 MHz, MeOD): δ 6.73 (m, 3H), 6.52 (d, J = 8.9 Hz, 2H), 6.10 (d, J = |
| | 7.0 Hz, 2H), 5.68 (d, J = 8.8 Hz, 2H), 5.40 (d, J = 14.9 Hz, 2H), 5.23 (s, 2H), |
| | 2.93 (s, 2H), 2.56 (m, 4H), 2.32 (s, 3H). LC/MS (ESI) m/z: 502.3 (M + H)⁺. RT |
| | (Method A): 0.75 min. |

### Scheme 90. Synthesis of (2S,4R)-4-(difluoromethoxy)-1-[2-[[3-[(5-ethyl-2-thienyl)oxy]benzoyl] amino]acetyl]-N-(1H-pyrrolo[3,2-c]pyridin-2-ylmethyl)pyrrolidine-2-carboxamide (Compound 368)

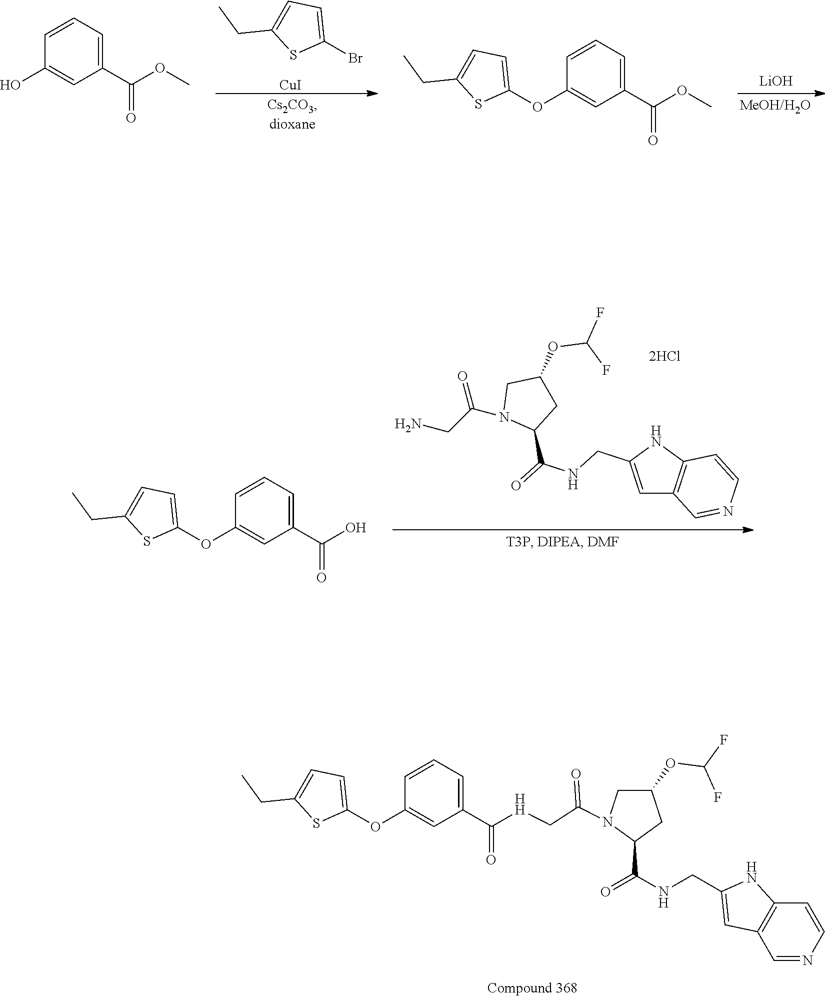

### Step 1: methyl 3-[(5-ethyl-2-thienyl)oxy]benizoate (2)

A mixture of methyl 3-hydroxybenzoate (50.00 mg, 0.3286 mmol, 1.0 equiv.), 2-bromo-5-ethyl-thiophene (0.1256 g, 0.6573 mmol, 2 equiv.), N,N-dimethylglycine hydrochloride (9.174 mg, 0.06573 mmol, 0.2 equiv.), CuI (6.259 m/g, 0.03286 mmol, 0.1 equiv.), Cs₂CO₃ (0.429 g, 1.32 mmol, 4 equiv.) in 1,4-dioxane (3 mL) was stirred in a microwave reactor at 110° C. for 12 hours. The reaction was cooled to room temperature, diluted with water, extracted with EtOAc, washed with brine, dried over Na₂SO₄ and concentrated. Purification by combi-Flash; 12 g column, solvent A=hexanes, solvent B=EtOAc. 100% A to 15% B to give methyl 3-[(5-ethyl-2-thienyl)oxy]benzoate (17.00 mg, 0.06481 mmol, 100 mass %, 19.72% Yield). LC/MS (ESI) m/z:263 (M+H)⁺.

### Step 2: 3-[(5-ethyl-2-thienyl)oxy]benzoic acid (3)

To a solution of methyl 3-[(5-ethyl-2-thienyl)oxy]benzoate (17.00 mg, 0.06481 mmol, 1.00 equiv.) in MeOH (1.5 mL) water (0.5 mL) was added LiOH (3.104 mg, 0.1296 mmol, 2 equiv.). The reaction was stirred at room temperature overnight. The reaction was then concentrated to remove McOH. The pH was adjusted to 1 using 1 N HCl. The acidified mixture was extracted with EtOAc. The combined organic extracts were washed with brine, dried over MgSO₄, and concentrated to give 3-[(5-ethyl-2-thienyl)oxy]benzoic acid (12.00 mg, 0.04833 mmol, 74.57% Yield) as a yellow sticky oil. LC/MS (ESI) m/z: 249 (M+H)⁺.

### Step 3: (2S,4R)-4-(difluoromethoxy)-1-[2-[[3-[(5-ethyl-2-thienyl)oxy]benzoyl]amino]acetyl]-N-(1H-pyrrolo[3,2-c]pyridin-2-ylmethyl)pyrrolidine-2-carboxamide (Compound 368)

To a solution of 3-[(5-ethyl-2-thienyl)oxy]benzoic acid (6.203 mg, 0.02498 mmol, 1.1 equiv.) in DMF (2 mL) was added (2S,4R)-1-(2-aminoacetyl)-4-(difluoromethoxy)-N-(1H-pyrrolo[3,2-c]pyridin-2-ylmethyl)pyrrolidine-2-carboxamide dihydrochloride (10.00 mg, 0.02271 mmol, 1.0 equiv.), T3P (50 mass %) in EtOAc (20.40 μL, 0.0341 mmol, 1.5 equiv.), and DIPEA (11.90 μL, 0.0682 mmol, 3.0 equiv.). The reaction mixture was stirred at room temperature for 30 minutes and then purified by directly prep HPLC to give Compound 368 (2.129 mg, 0.003563 mmol, 15.69% Yield). LC/MS (ESI) m/z: 598 (M+H). ¹H NMR (400 MHz, DMSO-d6): δ 11.21 (s, 1H), 8.94 (s, 1H), 8.78 (t, J=6.0 Hz, 1H), 8.73-8.61 (m, 1H), 8.35 (s, 1H₁), 8.30 (d, J=5.2 Hz, 1H), 8.23-8.13 (m, 1H), 7.96 (d, J=5.6 Hz, 1H), 7.82-7.67 (m, 1H), 7.59-7.48 (m, 1H), 7.18 (d, J=5.7 Hz, 1H), 7.00 (s, 1H), 6.81 (s, 1H), 6.63 (s, 1H), 4.98 (s, 1H), 4.51 (d, J=6.0 Hz, 1H), 4.49-4.33 (m, 1H), 4.24 (dd, J=17.0, 5.9 Hz, 1H), 4.07 (dd. J=16.9, 5.6 Hz, 1H), 3.90 (dd, J=11.6, 4.6 Hz, 1H), 3.85-3.73 (m, 1H), 2.77-2.59 (m, 11H), 2.42-2.27 (m, 1H), 2.16 (dt, J=13.2, 6.7 Hz, 1H), 1.3-1.89 (m, 3H).

### Scheme 91. Synthesis of (2S,4R)-N-((1H-pyrrolo[3,2-c]pyridin-2-yl)methyl)-1-((4-(cyclohexyloxy)benzoyl)glycyl)-4-(difluoromethoxy)pyrrolidine-2-carboxamide (Compound 383)

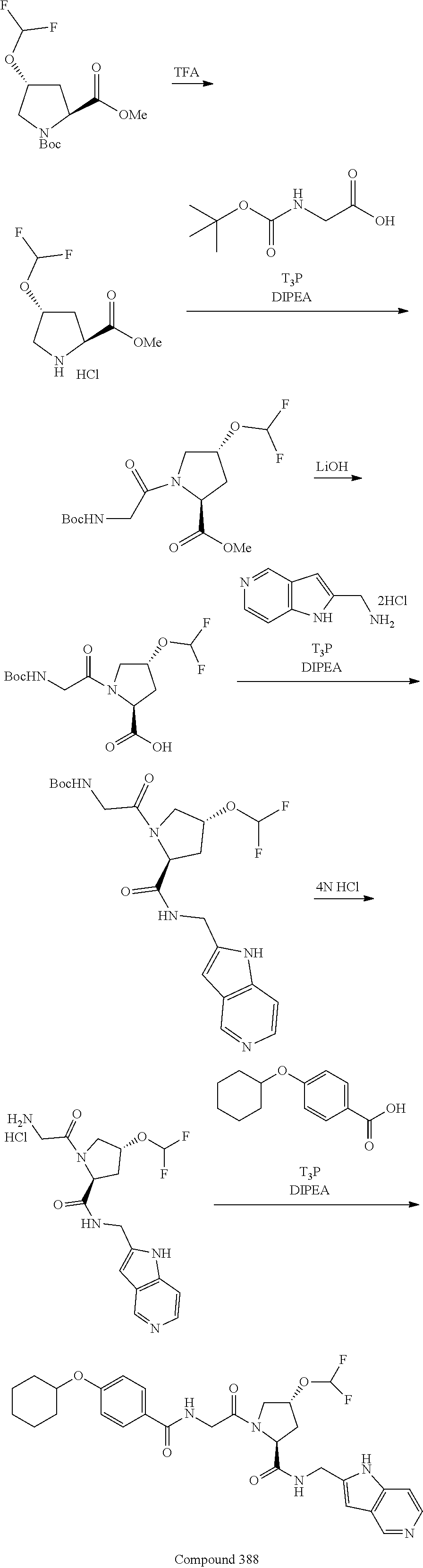

### Step 1: Methyl (2S,4R)-4-(difluoromethoxy)pyrrolidine-2-carboxylate hydrochloride

To a solution of 1-(tert-butyl) 2-methyl (2S,4R)-4-(difluoromethoxy)pyrrolidine-1,2-dicarboxylate (4.8 g, 16 mmol) in DCM (5.3 mL) was added TFA (5.3 mL, 81 mmol) at room temperature. After 1 hour, the volatiles were removed by concentration under reduced pressure to give methyl (2S,4R)-4-(difluoromethoxy)pyrrolidine-2-carboxylate hydrochloride (4.5 g, 120% yield). This material was used in next step without further purification. LC/MS (ESI) m/z: 196 (M+H)⁺ (free base).

### Step 2: Methyl (2S,4R)-1-((tert-butoxycarbonyl)glycyl)-4-(difluoromethoxy)pyrrolidine-2-carboxylate

To a solution of methyl (2S,4R)-4-(difluoromethoxy)pyrrolidine-2-carboxylate hydrochloride (3.8 g, 16 mmol), 2-(tert-butoxycarbonylamino)acetic acid (3 g, 17.1 mmol) and DIPEA (11 mL, 63.2 mmol) in DMF (50 mL) was added 50% T₃P in DMF (13 mL, 21.8 mmol) at 0° C. The reaction was stirred at 0° C. for 1 hour, then warmed to room temperature. H₂O (50 mL) was added, the reaction mixture was extracted with EtOAc. The combined organic layers were washed with saturated brine, dried over Na₂SO₄, filtered and concentrated to give methyl (2S,4R)-1-((tert-butoxycarbonyl)glycyl)-4-(difluoromethoxy)pyrrolidine-2-carboxylate (5.7 g, 99% yield) as a yellow oil. This material was used in next step without further purification. LC/MS (ESI) m/z: 353 (M+H)⁺.

### Step 3: (2S,4R)-1-((tert-Butoxycarbonyl)glycyl)-4-(difluoromethoxy)pyrrolidine-2-carboxylic acid

To a solution of methyl (2S,4R)-1-((tert-butoxycarbonyl)glycyl)-4-(difluoromethoxy)pyrrolidine-2-carboxylate (5.7 g, 16 mmol) in 1 to 1 to 1 ratio of MeOH/THF/H₂O (35 mL) was added LiOH monohydrate (2.3 g, 55 mmol) at room temperature. The mixture was stirred for overnight. The mixture was washed with MTBE (50 mL) and the organic layer was discarded. The aqueous layer was acidified to pH=3 with IN HCl and extracted with EtOAc (3×50 mL). The combined organic layers were washed with saturated brine, dried over Na₂SO₄, filtered, and concentrated to give (2S,4R)-1-((tert-butoxycarbonyl)glycyl)-4-(difluoromethoxy)pyrrolidine-2-carboxylic acid (2.6 g, 48% yield) as a clear oil. LC/MS (ESI) n/z: 339 (M+H)⁺.

### Step 4: tert-Butyl (2-((2S,4R)-2-(((1H-pyrrolo[3,2-c]pyridin-2-yl)methyl)carbamoyl)-4-(difluoromethoxy)pyridin-1-yl)-2-oxoethyl)carbamate

To a solution of (2S,4R)-1-((tert-butoxycarbonyl)glycyl)-4-(difluoromethoxy)pyrrolidine-2-carboxylic acid (150 mg, 0.44 nmol), (1H-pyrrolo[3.2-c]pyridin-2-yl)methanamine dihydrochloride (100 mg, 0.45 nmol), and 50% T₃P in DMF (0.4 mL, 0.7 mmol) in DMF (20 mL) was added DIPEA (0.39 mL, 2.2 mmol) at 0° C. The mixture was warmed to room temperature for 2 hours and quenched with H₂O (20 mL). The mixture was extracted with EtOAc (3×50 mL). The organic layers were concentrated and purified on silica gel column chromatography (0 to 10% MeOH in DCM) to give tert-butyl (2-((2S,4R)-2-(((1H-pyrrolo[3,2-c]pyridin-2-yl)methyl)carbamoyl)-4-(difluoromethoxy)pyrrolidin-1-yl)-2-oxoethyl)carbamate (130 mg, 63% yield). LC/MS (ESI) m/z: 468 (M+H)⁺.

### Step 5: (2S,4R)-N-((1H-pyrrolo[3,2-c]pyridin-2-yl)methyl)-4-(difluoromethoxy)-1-glycylpyrrolidine-2-carboxamide hydrochloride

To a solution of tert-butyl (2-((2S,4R)-2-(((1H-pyrrolo[3,2-c]pyridin-2-yl)methyl)carbamoyl)-4-(difluoromethoxy)pyrrolidin-1-yl)-2-oxoethyl)carbamate (130 mg, 0.28 mmol) in EtOAc (5.5 mL) was added 4N HCl in 1,4-dioxane (0.21 mL) at room temperature. Yellow solid crushed out immediately. MeOH (1 mL) was added to give a yellow solution. The reaction was stirred at room temperature for 72 hours. The volatiles were removed by concentration and the mixture was suspended in MTBE (20 mL). The solid was filtered and dried to give (2S,4R)-N-((1H-pyrrolo[3,2-c]pyridin-2-yl)methyl)-4-(difluoromethoxy)-1-glycylpyrrolidine-2-carboxamide hydrochloride (100 mg, 89% yield). LC/MS (ESI) m/z: 404 (M+H)⁺.

### Step 6: (2S,4R)-N-((1H-pyrrolo[3,2-c]pyridin-2-yl)methyl)-1-((4-(cyclohexyloxy)benzoyl)glycyl)-4-(difluoromethoxy)pyrrolidine-2-carboxamide (Compound 383)

To a solution of (2S,4R)-N-((1H-pyrrolo[3,2-c]pyridin-2-yl)methyl)-4-(difluoromethoxy)-1-glycylpyrrolidine-2-carboxamide hydrochloride (10 mg, 0.025 mmol), DIPEA (0.022 mL, 0.13 mmol), and 4-(cyclohexoxy)benzoic acid (6 mg, 0.027 mmol) in DMF (0.5 mL) was added 50% T₃P in DMF (0.022 mL, 0.037 mmol) at room temperature. The reaction was stirred at room temperature for overnight. The mixture was directly purified by silica gel column chromatography (silica gel, 0 to 100% of 10% 7N NH₃ in methanol in DCM in DCM) to give Compound 383 (2.3 mg, 16% yield) as a white solid. ¹H-NMR (400 MHz, DMSO-d6) δ 11.29 (d, J=77.5 Hz, 1H), 9.00-8.35 (m, 2H), 8.27-7.72 (m, 2H), 7.44-6.30 (m, 3H), 4.92 (d, J=35.6 Hz, 1H), 4.61-4.37 (m, 1H), 4.26-3.94 (m, 1H), 3.94-3.69 (m, 2H), 2.24 (d, J=63.8 Hz, 2H), 1.83 (d, J=88.2 Hz, 2H), 1.62-0.98 (m, 6H). LC/MS (ESI) m/z: 570 (M+H)⁺. RT (Method A): 1.55 min.

### Scheme 92. Synthesis of (2S,4R)-N-((1H-pyrrolo[3,2-c]pyridin-2-yl)methyl)-4-(difluoromethoxy)-1-((3-(2-fluoro-4-methylbenzyl)benzoyl)glycyl)pyrrolidine-2-carboxamide (Compound 305)

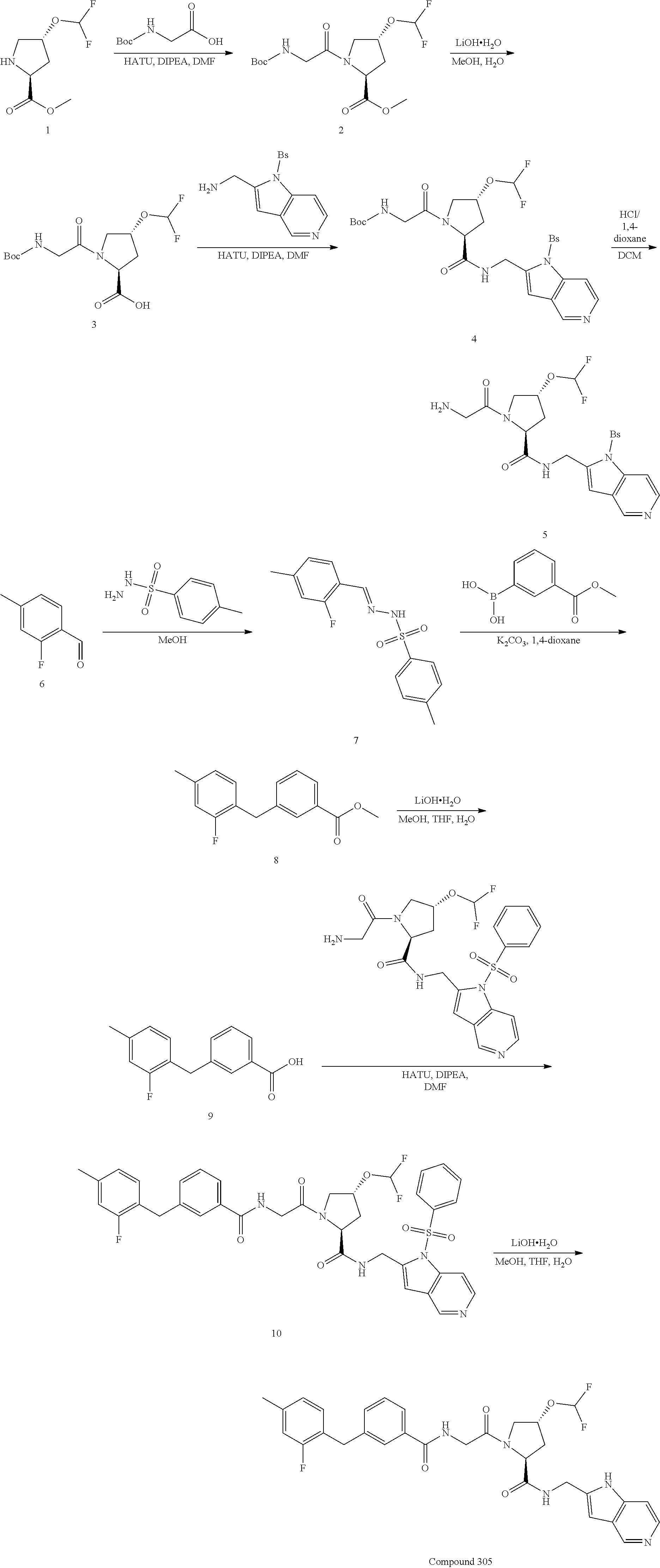

### Step 1: Methyl (2S,4R)-1-((tert-butoxycarbonyl)glycyl)-4-(difluoromethoxy) pyrrolidine-2-carboxylate (2)

To a mixture of methyl (2S,4R)-4-(difluoromethoxy)pyrrolidine-2-carboxylate (500 mg, 2.56 mmol) and (tert-butoxycarbonyl)glycine (538 mg, 3.07 mmol) in DMF (5 mL) was added DIPEA (1653 mg, 12.79 mmol) and IIATU (1169 mg, 3.07 mmol) under N₂ atmosphere and the reaction mixture was stirred at room temperature for 2 hours. The mixture was diluted with EtOAc, washed with saturated aq·NaHCO₃ solution, dried over anhydrous Na₂SO₄, filtered, and concentrated under reduced pressure to dryness. The residue was purified by flash chromatography (silica gel, 0-5% MeOH in DCM) to give the title compound (500 mg, yield 55.4%) as a yellow oil. LC/MS (ESI) m/z: 353 (M+H)⁺.

### Step 2: (2S,4R)-1-((Tert-butoxycarbonyl)glycyl)-4-(difluoromethoxy)pyrrolidine-2-carboxylic acid (3)

To a solution of methyl (2S,4R)-1-((tert-butoxycarbonyl)glycyl)-4-(difluoromethoxy) pyrrolidine-2-carboxylate (500 mg, 1.42 mol) in MeOH/water (5 mL, v/v=4/1) was added LiOH·H₂O (120 mg, 2.86 mmol) under N₂ atmosphere and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (420 mg, yield 87.4%) as a yellow solid, which was used directly in the next step without further purification. LC/MS (ESI) m/z: 339 (M+H)⁺.

### Step 3: Tert-butyl (2-((2S,4R)-2-(((1-((4-bromophenyl)sulfonyl)-1H-pyrrolo[3,2-c]pyridin-2-yl)methyl)carbamoyl)-4-(difluoromethoxy)pyrrolidin-1-yl)-2-oxoethyl) carbamate (4)

To a mixture of (2S,4R)-1-((tert-butoxycarbonyl)glycyl)-4-(difluoromethoxy) pyrrolidine-2-carboxylic acid (420 mg, 1.24 mmol) and (1-((4-bromophenyl)sulfonyl)-1H-pyrrolo[3,2-c]pyridin-2-yl)methanamine (544 mg, 1.67 mmol) in DMF (5 mL) was added DIPEA (801 mg, 6.20 mmol) and HATU (566 mug, 1.49 mmol) under N₂ atmosphere and the reaction mixture was stirred at room temperature for 1 hour. The mixture was concentrated under reduced pressure to dryness. The residue was purified by flash chromatography (silica gel, 0-10% MeOH in DCM) to give the title compound (635 mg, yield 74.5%) as a yellow oil. LC/MS (ESI) m/z: 608 (M+H)⁺.

### Step 4: (2S,4R)-N-((1-((4-bromophenyl)sulfonyl)-JH-pyrrolo[3,2-c]pyridin-2-yl) methyl)-4-(difluoromethoxy)-1-glycylpyrrolidine-2-carboxamide hydrochloride (5)

To a solution of tert-butyl (2-((2S,4R)-2-(((1-((4-bromophenyl)sulfonyl)-1H-pyrrolo[3,2-c]pyridin-2-yl)methyl)carbamoyl)-4-(difluoromethoxy)pyrrolidin-1-yl)-2-oxoethyl)carbamate (635 mg, 1.04 mmol) in DCM (3 mL) was added HCl/1,4-dioxane (3 mL, 4M) and the reaction mixture was stirred under N₂ atmosphere at room temperature for 2 hours. The reaction mixture was concentrated to dryness under reduced pressure to give the title compound (540 mg, yield 99.5%) as a yellow solid, which was used directly in the next step without further purification LC/MS (EST) m/z: 508 (M+H)⁺.

### Step 5: (E)-N′-(2-fluoro-4-methylbenzylidene)-4-methylbenzenesulfonohydrazide (7)

To a solution of 2-fluoro-4-methylbenzaldehyde (500 mg, 3.6 mmol) in MeOH (8 mL) was added 4-methylbenzenesulfonohydrazide (674 mg. 3.6 mmol) under N₂ atmosphere and the reaction mixture was stirred at room temperature for 16 hours. The mixture was filtered, and the filter cake was dried under vacuum to give the title compound (930 mg, yield 83.9%) as a white solid. LC/MS (ESI) m/z: 307 (M+H)⁺.

### Step 6: Methyl 3-(2-fluoro-4-methylbenzyl)benzoate (8)

To a mixture of (E)-N′-(2-fluoro-4-methylbenzylidene)-4-methylbenzenesulfonohydrazide (800 mg, 2.6 mmol) and (3-(methoxycarbonyl)phenyl) boronic acid (565 mg, 3.1 mmol) in 1,4-dioxane (10 mL) was added K₂CO₃ (1.1 g, 7.8 mmol) at 25° C. under N₂ atmosphere and the reaction mixture was stirred at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (470 mg, yield 69.7%) as a colorless oil. LC/MS (ESI) m/z: 259 (M+H)⁺.

### Step 7: 3-(2-Fluoro-4-methylbenzyl)benzoic acid (9)

To a solution of methyl 3-(2-fluoro-4-methylbenzyl)benzoate (550 mg, 2.1 mmol) in THF/MeOH/H₂O (6 mL, v/v/v=2/1/1) was added LiOH·H₂O (269 mg, 6.4 mmol) under N₂ atmosphere and the reaction mixture was stirred at room temperature for 2 hours. The mixture was acidified with IN aq·HCl to pH˜3 and extracted with EtOAc twice. The combined organic layers were dried over anhydrous Na₂SO₄, filtered, and concentrated under reduced pressure to give the title compound (60 mg, yield 88.3%) as a white solid, which was used directly in the next step without further purification. LC/MS (ESI) m/z: 243 (M+H)⁺.

### Step 8: (2S,4R)-4-(difluoromethoxy)-1-((3-(2-fluoro-4-methylbenzyl)benzoyl)glycyl)-N-((1-(phenylsulfonyl)-1H-pyrrolo[3,2-c]pyridin-2-yl)methyl)pyrrolidine-2-carboxamide (10)

To a mixture of 3-(2-fluoro-4-methylbenzyl)benzoic acid (15 mg, 0.06 mmol) and (2S,4R)-4-(difluoromethoxy)-1-glycyl-N-((1-(phenylsulfonyl)-1H-pyrrolo[3,2-c]pyridin-2-yl)methyl)pyrrolidine-2-carboxamide (31 mg, 0.06 mmol) in DMF (2 mL) was added DIPEA (39 mg, 0.31 mmol) and HATU (30 mg, 0.08 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=15:1) to give the title compound (20 mg, yield 44.4%) as a white solid. LC/MS (ESI) m/z: 734 (M+H)⁺.

### Step 9: (2S,4R)-N-((1H-pyrrolo[3, 2-c]pyridin-2-yl)methyl)-4-(difluoromethoxy)-1-((3-(2-fluoro-4-methylbenzyl)benzoyl)glycyl)pyrrolidine-2-carboxamide (Compound 305)

To a solution of (2S,4R)-4-(difluoromethoxy)-1-((3-(2-fluoro-4-methylbenzyl) benzoyl)glycyl)-N-((1-(phenylsulfonyl)-1H-pyrrolo[3,2-c]pyridin-2-yl)methyl)pyrrolidine-2-carboxamide (20 mg, 0.03 mmol) in THF/MCOH/H₂O (2 mL, v/v/v=2/1/1) was added LiOH Fit (269 mg, 6.4 mmol) under N₂ atmosphere and the reaction mixture was stirred at room temperature for 2 hours. The mixture was concentrated under reduced pressure to give Compound 305 (1.6 mg, yield 9.9%) as a white solid. ¹H NMR (400 MHz, CD₃OD): δ 8.73 (s, 1H), 7.95 (d, J=6.0 Hz, 1H), 7.73 (s, 1H), 7.68 (d, J=7.1 Hz, 1H), 7.40-7.36 (m, 2H), 7.32 (d, J=6.4 Hz, 1H), 7.08 (t, J=8.0 Hz, 1H), 6.90-6.84 (m, 2H), 6.71-6.34 (m, 2H), 5.04 (s, 1H), 4.72 (d, J=15.6 Hz, 1H), 4.59 (t, J=8.0 Hz, 2H), 4.22 (d, J=2.6 Hz, 2H), 4.00 (d, J=8.4 Hz, 3H), 3.89 (d, J=11.2 Hz, 1H), 2.54-2.46 (m, 1H), 2.30 (s, 1H), 2.28 (s, 3H). LC/MS (ESI) m/z: 594 (M+H)⁺. RT (Method A): 1.61 min.

### Scheme 93. Synthesis of (S)-N-((1H-pyrrolo[3,2-c]pyridin-2-yl)methyl)-7-((3,6-dimethyl-2-(m-tolyl)isonicotinoyl)glycyl)-1,4-dioxa-7-azaspiro[4.4]nonane-8-carboxamide (Compound 306)

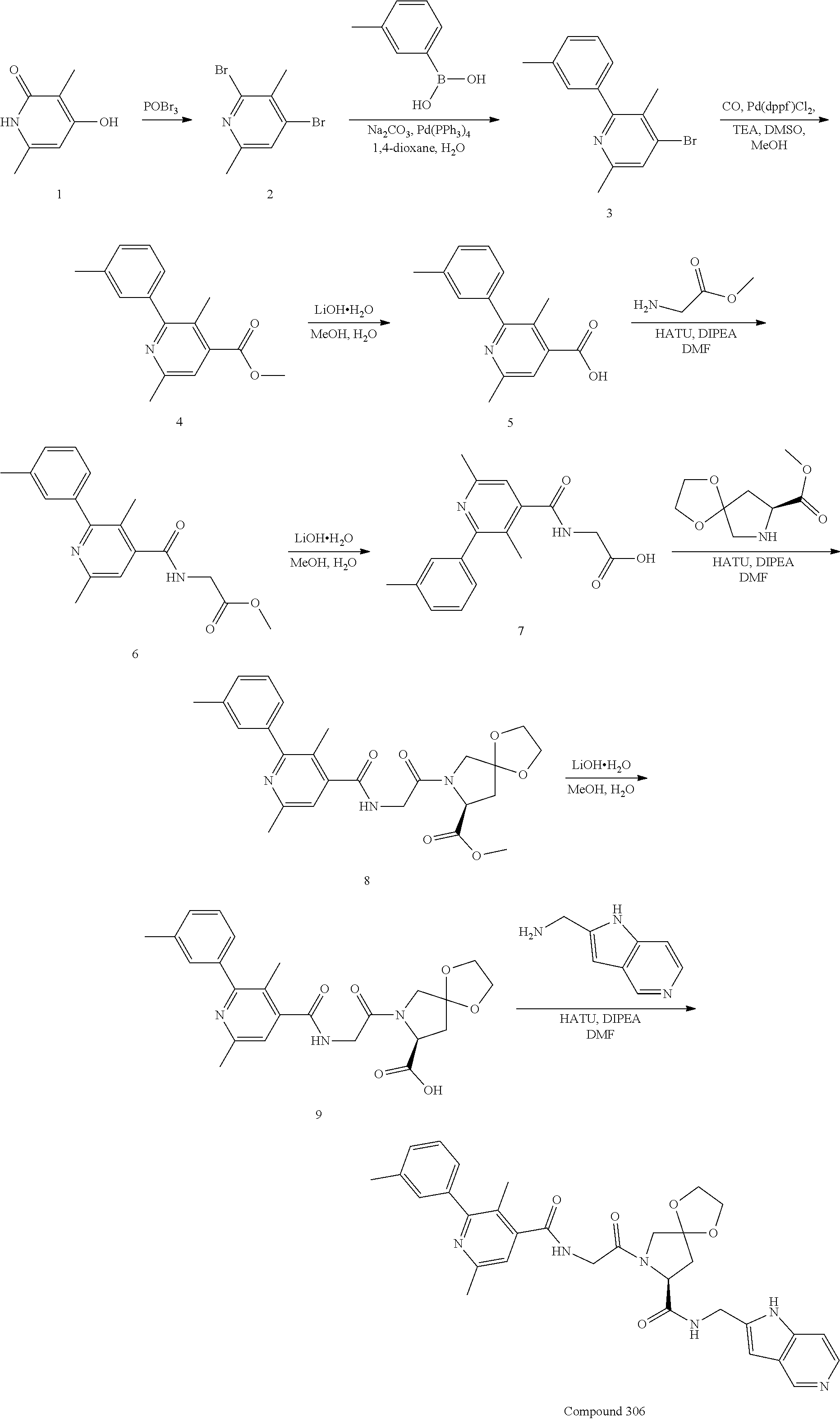

### Step 1: 2, 4-Dibromo-3, 6-dimethylpyridine (2)

A mixture of 4-hydroxy-3,6-dimethylpyridin-2(1H)-one (800 mg, 5.75 mmol) and POBr₃ (5.0 g, 17.5 mmol) was stirred at 150° C. for 6 hours. The mixture was quenched with ice water and basified with 1 M aq·NaOH to pH˜8. The mixture was extracted with EtOAc twice and the combined organic layers were washed with water and brine, dried over anhydrous Na₂SO₄, filtered, and concentrated under reduced pressure to dryness. The residue was purified by flash chromatography (silica gel, 0-9% EtOAc in PE) to give the title compound (570 mg, yield 37.7%) as a white solid. LC/MS (ESI) (m/z): 264 (M+H)⁺.

### Step 2: 4-Bromo-3, 6-dimethyl-2-(m-tolyl)pyridine (3)

To a mixture of 2,4-dibromo-3,6-dimethylpyridine (530 mg, 2.02 mmol) and m-tolylboronic acid (274 mg, 2.02 mmol) in 1,4-dioxane (6 mL) and water (1 mL) was added Na₂CO₃ (641 mg, 6.06 mmol) and Pd(PPh₃)₄ (233 mg, 0.20 mmol) under N₂ atmosphere, the reaction mixture was degassed under N₂ atmosphere for three times and stirred at 80° C. overnight. The mixture was diluted with EtOAc, washed with brine, dried over anhydrous Na₂SO₄, filtered, and concentrated under reduced pressure to dryness. The residue was purified by flash chromatography (silica gel, 0-10% EtOAc in PE) to give the title compound (320 mg, yield 57.8%) as a colorless oil. LC/MS (ESI) m/z: 276 (M+H)⁺.

### Step 3: Methyl 3, 6-dimethyl-2-(m-tolyl)isonicotinate (4)

To a solution of 4-bromo-3,6-dimethyl-2-(m-tolyl)pyridine (320 mg, 1.16 mmol) in MeOH (2 mL) and DMSO (2 mL) was added TEA (353 mg, 3.49 mmol) and Pd(dppf)Cl₂ (85 mg, 0.12 mmol). The reaction mixture was degassed under CO atmosphere for three times and the reaction mixture was stirred at 85° C. overnight. The mixture was diluted with EtOAc, washed with brine, dried over anhydrous Na₂SO₄, filtered, and concentrated under reduced pressure to dryness. The residue was purified by flash chromatography (silica gel, 0-5% EtOAc in PE) to give the title compound (220 mg, yield 74.1%) as a colorless oil. LC/MS (ESI) m/z: 256 (M+H)⁺.

### Step 4: 3, 6-Dimethyl-2-(m-tolyl)isonicotinic acid (5)

To a solution of methyl 3,6-dimethyl-2-(m-tolyl)isonicotinate (220 mg, 0.86 mmol) in MeOH (3 mL) and water (1 mL) was added LiOH·H₂O (109 mg, 2.58 mmol) and the mixture was stirred at room temperature for 2 hours. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (160 mg, yield 76.9%) as a white solid, which was used directly in the next step without further purification. LC/MS (ESI) m/z: 242 (M+H)⁺.

### Step 5: Methyl (3,6-dimethyl-2-(m-tolyl)isonicotinoyl)glycinate (6)

To a mixture of 3,6-dimethyl-2-(m-tolyl)isonicotinic acid (100 mg, 0.41 mmol) and methyl glycinate hydrochloride (104 mg, 0.82 mmol) in DMF (3 mL) was added DIPEA (321 mg, 2.49 mmol) and H ATU (189 mg, 0.49 mmol) under N₂ atmosphere and the reaction mixture was stirred at room temperature for 2 hours. The mixture was diluted with EtOAc, washed with brine, dried over anhydrous Na₂SO₄, filtered, and concentrated under reduced pressure to dryness. The residue was purified by flash chromatography (silica gel, 0-70% EtOAc in PE) to give the title compound (90 mg, yield 69.5%) as a white solid. LC/MS (ESI) m/z: 313 (M+H)⁺.

### Step 6: (3,6-Dimethyl-2-(m-tolyl)isonicotinoyl)glycine (7)

To a solution of methyl (3,6-dimethyl-2-(m-tolyl)isonicotinoyl)glycinate (90 mg, 0.29 mmol) in MeOH (3 mL) and water (1 mL) was added LiOH H₂O (36 mg, 0.87 mmol) and the mixture was stirred at room temperature for 2 hours. The mixture was acidified with 1N aq·HCl to pH˜3 and extracted with CHCl₃/i-PrOH (v/v=3/1) twice. The combined organic layers were washed with brine, dried over anhydrous Na₂SO₄, filtered, and concentrated under reduced pressure to dryness to give the title compound (75 mug, yield 87.3%) as a white solid, which was used directly in the next step without further purification. LC/MS (ESI) m/z: 299 (M+H)⁺.

### Step 7: Methyl (S)-7-((3,6-dimethyl-2-(m-tolyl)isonicotinoyl)glycyl)-1,4-dioxa-7-azaspiro[4.4]nonane-8-carboxylate (8)

To a mixture of (3,6-dimethyl-2-(m-tolyl)isonicotinoyl)glycine (50 mg, 0.17 mmol) and methyl (S)-1,4-dioxa-7-azaspiro[4.4]nonane-8-carboxylate (47 mg, 0.25 mmol) in DMF (3 mL) was added DIPEA (130 mg, 1.02 mmol) and HATU (76 mg, 0.20 mmol) under N₂ atmosphere and the reaction mixture was stirred at room temperature for 2 hours. The mixture was diluted with EtOAc, washed with brine, dried over anhydrous Na₂SO₄, filtered, and concentrated under reduced pressure to dryness. The residue was purified by flash chromatography (silica gel, 0-90% EtOAc in PE) to give the title compound (50 mg, yield 63.8%) as a white solid. LC/MS (ESI) m/z: 468 (M+H)⁺.

### Step 8: (S)-7-((3,6-dimethyl-2-(m-tolyl)isonicotinoyl)glycyl)-1,4-dioxa-7-azaspiro[4.4]nonane-8-carboxylic acid (9)

To a solution of methyl (S)-7-((3,6-dimethyl-2-(m-tolyl)isonicotinoyl)glycyl)-1,4-dioxa-7-azaspiro[4.4]nonane-8-carboxylate (50 mg, 0.11 mmol) in MeOH (3 mL) and water (1 mL) was added LiOH·H₂O (13 mg, 0.32 mmol) and the mixture was stirred at room temperature for 2 hours. The mixture was acidified with 1N aq·HCl to pH˜3 and extracted with CHCl₃/i-PrOH (v/v/=3/1) twice. The combined organic layers were washed with brine, dried over anhydrous Na₂SO₄, filtered, and concentrated under reduced pressure to dryness to give the title compound (40 mg, yield 82.5%) as a brown solid, which was used directly in the next step without further purification. LC/MS (ESI) m/z: 454 (M+H)⁺.

### Step 9: (S)-N-((1H-pyrrolo[3, 2-c]pyridin-2-yl)methyl)-7-((3,6-dimethyl-2-(m-tolyl) isonicotinoyl)glycyl)-1,4-dioxa-7-azaspiro[4.4]nonane-8-carboxamide (Compound 306)

To a mixture of (S)-7-((3,6-dimethyl-2-(m-tolyl)isonicotinoyl)glycyl)-1,4-dioxa-7-azaspiro[4.4]nonane-8-carboxylic acid (40 mg, 0.09 mmol) and (1H-pyrrolo[3,2-c]pyridin-2-yl)methanamine (26 mg, 0.18 mmol) in DMF (2 mL) was added DIPEA (68 mg, 0.54 mmol) and HATU (40 mg, 0.11 mmol) at 0° C. under N₂ atmosphere and the mixture was stirred at room temperature for 1 hour. The mixture was extracted with CHCl₃/i-PrOH (v/v=3/1) twice. The combined organic layers were washed with brine, dried over anhydrous Na₂SO₄, filtered, and concentrated under reduced pressure to dryness. The residue was purified by prep-TLC (DCM:MeOH=9:1) and further purified by prep-HPLC to give Compound 306 (10 mg, yield 19.4%) as a white solid. ¹H NMR (400 MHz, CD₃OD): δ 8.84 (s, 1H), 8.47 (s, 1H), 8.14 (d, J=6.3 Hz, 1H), 7.60 (d, J=6.2 Hz, 1H), 7.37 (t, J=7.6 Hz, 1H), 7.27 (d, J=4.2 Hz, 2H), 7.23 (s, 1H), 7.19 (d, J=7.6 Hz, 1H), 6.79 (s, 1H), 4.66 (s, 1H), 4.62 (d, J=8.7 Hz, 2H), 4.22 (s, 2H), 4.01 (d, J=2.0 Hz, 4H), 3.81 (s, 2H), 2.52 (s, 3H), 2.49-2.43 (m, 1H), 2.41 (s, 3H), 2.30-2.25 (m, 1H), 2.21 (s, 3H). LC/MS (ESI) (m/z): 583 (M+H)⁺. RT (Method A): 0.79 min.

### Scheme 94. Synthesis of (S)-N-((1H-pyrrolo[3,2-c]pyridin-2-yl)methyl)-7-((3-(2-fluoro-4-methylphenoxy)benzoyl)glycyl)-1,4-dioxa-7-azaspiro[4.4]nonane-8-carboxamide (Compound 307)

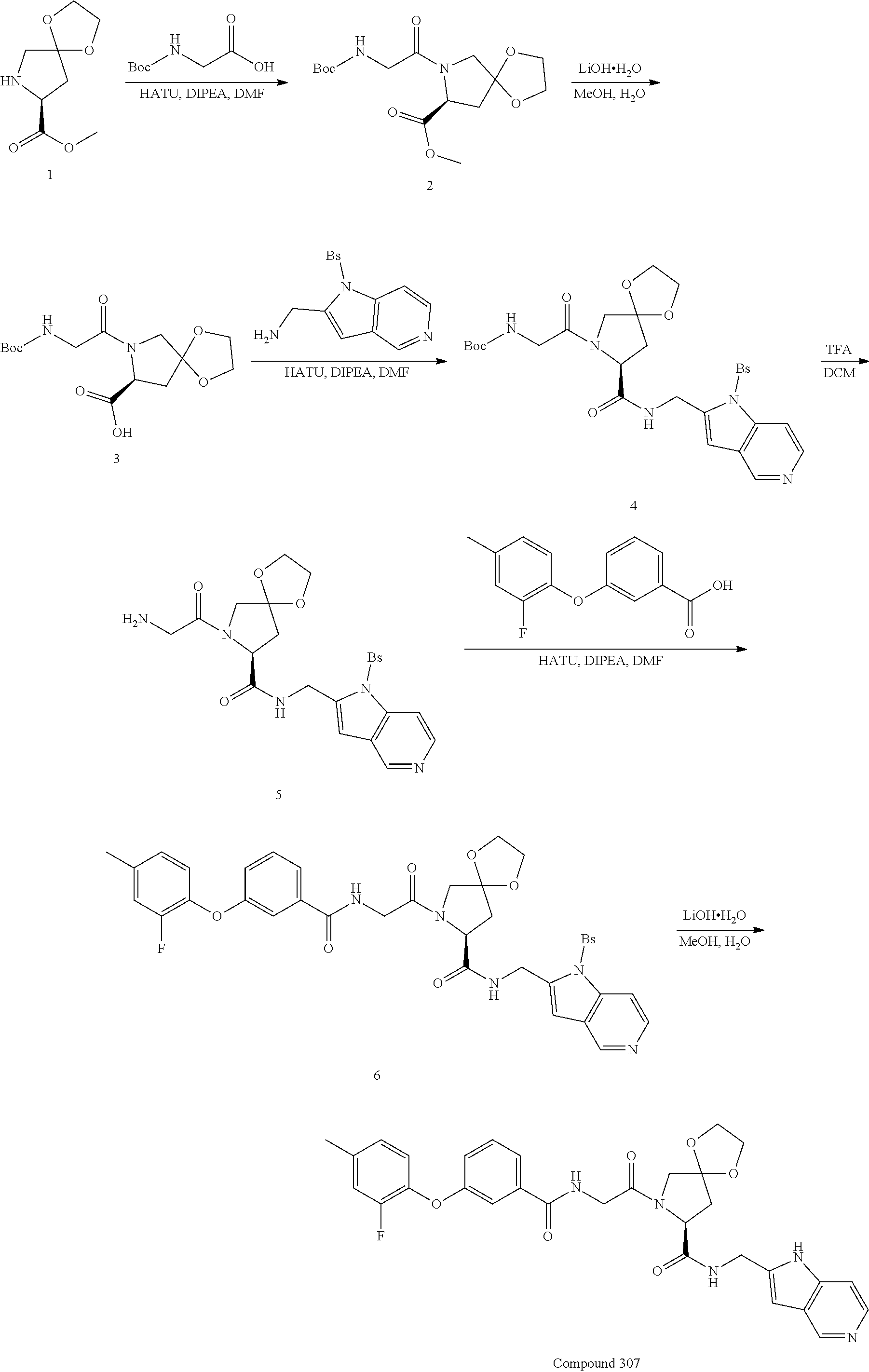

### Step 1: Methyl (S)-7-((tert-butoxycarbonyl)glycyl)-1,4-dioxa-7-azaspiro[4.4]nonane-8-carboxylate (2)

To a mixture of (tert-butoxycarbonyl)glycine (370 mg, 2.11 mmol) and methyl (S)-1,4-dioxa-7-azaspiro[4.4]nonane-8-carboxylate (400 mg, 2.14 mmol) in DMF (10 mL) was added HATU (975 mg, 2.56 mmol) and DIPEA (827 mg, 6.41 mmol). The reaction mixture was stirred at 25° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (300 mg, yield 41.2%) as a white solid. LC/MS (ESI) m/z: 345 (M+H)⁺.

### Step 2: (S)-7-((tert-butoxycarbonyl)glycyl)-1,4-dioxa-7-azaspiro[4.4]nonane-8-carboxylic acid (3)

To a solution of methyl (S)-7-((tert-butoxycarbonyl)glycyl)-1,4-dioxa-7-azaspiro[4.4]nonane-8-carboxylate (300 mg, 0.87 mmol) in THF/MeOH/H₂O (15 mL, v/v/v=1/1/1) was added LiOH·H₂O (109 mg, 2.60 mmol) and the mixture was stirred at 25° C. overnight. The mixture was acidified with 1N aq·HCl to pH˜3 and extracted with EtOAc twice. The combined organic layers were dried over anhydrous Na₂SO₄, filtered, and concentrated to dryness to give the title compound (200 mg, yield 69.5%) as a yellow solid, which was used in next reaction without purification. LC/MS (ESI) m/z: 331 (M+H)⁺.

### Step 3: Tert-butyl (S)-(2-oxo-2-(8-(((1-(phenylsulfonyl)-1H-pyrrolo[3, 2-c]pyridin-2-yl)methyl)carbamoyl)-1,4-dioxa-7-azaspiro[4.4]nonan-7-yl)ethyl)carbamate (4)

To a mixture of (S)-7-((tert-butoxycarbonyl)glycyl)-1,4-dioxa-7-azaspiro[4.4]nonane-8-carboxylic acid (100 mg, 0.30 mmol) and (1-(phenylsulfonyl)-1H-pyrrolo[3,2-c]pyridin-2-yl)methanamine (110 mg, 0.30 mmol) in DMF (3 mL) was added DIPEA (78 mg, 0.61 mmol) and H ATU (115 mg, 0.30 mmol) under N₂ atmosphere and the reaction mixture was stirred at room temperature for 2 hours. The mixture was diluted with EtOAc, washed with saturated aq·NH₄Cl solution, dried over anhydrous Na₂SO₄, filtered, and concentrated under reduced pressure to dryness. The residue was purified by flash chromatography (silica gel, 0-5% MeOH in DCM) to give the title compound (100 mg, yield 71.9%) as a white solid. LC/MS (ESI) m/z: 600 (M+H)⁺.

### Step 4: (S)-7-glycyl-N-((1-(phenylsulfonyl)-1H-pyrrolo[3,2-c]pyridin-2-yl)methyl)-1,4-dioxa-7-azaspiro[4.4]nonane-8-carboxamide (5)

To a solution of tert-butyl (S)-(2-oxo-2-(8-(((1-(phenylsulfonyl)-1H-pyrrolo[3,2-c]pyridin-2-yl)methyl)carbamoyl)-1,4-dioxa-7-azaspiro[4.4]nonan-7-yl)ethyl)carbamate (100 mg, 0.17 mmol) in DCM (2 mL) was added TFA (1 mL) and the mixture was stirred at room temperature for 2 hours. The mixture was concentrated under reduced pressure to dryness to give title compound (110 mg, yield 100%) as a yellow oil, which was directly used in the next reaction without purification. LC/MS (ESI) m/z: 500 (M+H)⁺.

### Step 5: (S)-7-((3-(2 fluoro-4-methylphenoxy)benzoyl)glycyl)-N((1-(phenylsulfonyl)-Hpyrrolo[3,2-c]pyridin-2-yl)methyl)-1,4-dioxa-7-azaspiro[4.4]nonane-8-carboxamide (6)

To a solution of 3-(2-fluoro-4-methylphenoxy)benzoic acid (40 mg, 0.16 mmol) in DMF (2.0 mL) was added crude (S)-7-glycyl-N-((1-(phenylsulfonyl)-1H-pyrrolo[3,2-c]pyridin-2-yl)methyl)-1,4-dioxa-7-azaspiro[4.4]nonane-8-carboxamide (100 mg, 0.17 mmol), HATU (74 mg, 0.19 mmol) and DIEA (142 mg, 1.1 mmol) and the mixture was stirred at room temperature for 1 hour. The mixture was diluted with water and extracted with EtOAc twice. The combined organic layers were washed with brine, dried over anhydrous Na₂SO₄, filtered, and concentrated to dryness. The residue was purified by flash chromatography (silica gel, 0-5% MeOH in DCM) to give the title compound (60 mg, yield 50.8%) as a white solid. LC/MS (ESI) m/z: 728 (M+H)⁺.

### Step 6: (S)-N-((JH-pyrrolo[3,2-c]pyridin-2-yl)methyl)-7-((3-(2-fluoro-4-methylphenoxy)benzoyl)glycyl)-1,4-dioxa-7-azaspiro[4.4]nonane-8-carboxamide (Compound 307)

To a solution of (S)-7-((3-(2-fluoro-4-methylphenoxy)benzoyl)glycyl)-N-((1-(phenylsulfonyl)-1H-pyrrolo[3,2-c]pyridin-2-yl)methyl)-1,4-dioxa-7-azaspiro[4.4]nonane-8-carboxamide (60 mg, 0.08 mmol) in MeOH/H₂O (2 mL, v/v=2/1) was added LiOH·H₂O (10.4 mg, 0.25 mmol) and the mixture was stirred at 50° C. for 1 hour. The reaction mixture was concentrated under reduced pressure to dryness. The residue was purified by prep-HPLC to give Compound 307 (2.0 mg, yield 4.1%) as a white solid. ¹H NMR (400 MHz, CD₃OD): δ 8.63 (s, 1H), 8.63 (s, 1H), 7.99 (d, J=5.8 Hz, 1H), 7.53 (d, J=7.8 Hz, 1H), 7.43-7.38 (m, 2H), 7.21 (d, J=5.8 Hz, 1H), 7.11-7.04 (m, 2H), 6.99 (t, J=8.5 Hz, 2H), 6.50 (s, 1H), 4.81-4.60 (m, 2H), 4.60-4.43 (m, 2H), 4.18-4.01 (m, 1H), 4.01-3.96 (m, 4H). 3.80 (d, J=11.3 Hz, 2H), 2.46-2.40 (m, 1H), 2.34 (s, 3H), 2.29-2.23 (m, 1H). LC/MS (ESI) m/z: 588 (M+H)⁺. RT (Method A): 1.40 min.

The following compounds were prepared based on Scheme 94:

| Cmpd # | Reactant A | Reactant B | Reactant C |
|---|---|---|---|
| 309^{a} | 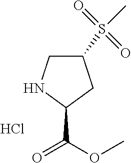 | 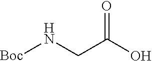 [SMILES: CC(C)(C)OC(=O)NCC(=O)O] | 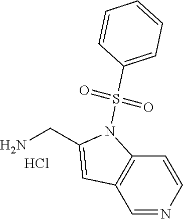 [SMILES: NCc1cc2cnccc2n1S(=O)(=O)c1ccccc1 ; Cl] |
| 312^{a} | 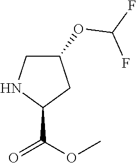 | 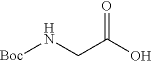 [SMILES: CC(C)(C)OC(=O)NCC(=O)O] | 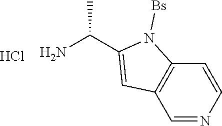 |
| 315 | 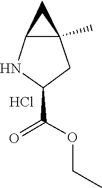 | 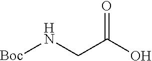 [SMILES: CC(C)(C)OC(=O)NCC(=O)O] | 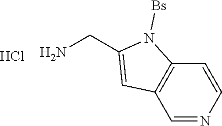 |

| Cmpd | | |
|---|---|---|
| # | Reactant D | Characterization Data |
| 309^{a} | 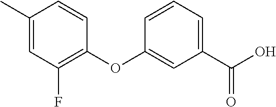 | ¹H NMR (400 MHz, CD₃OD): δ 8.84 (s, 1H), 8.47-8.39 (m, 1H), 8.12 (d, J = 6.3 Hz, 1H), 7.59 (d, J = 6.4 Hz, 1H), 7.53 (d, J = 7.7 Hz, 1H), 7.42-7.37 (m, 2H), 7.09-7.07 (m, 1H), 7.05-7.02 (m, 1H), 7.00 (s, 1H), 6.79 (s, 1H), 4.72 (d, J = 3.9 Hz, 1H), 4.65 (d, J = 13.3 Hz, 1H), 4.31 (d, J = 16.6 Hz, 1H), 4.18 (t, J = 8.2 Hz, 4H), 3.08 (s, 3H), 2.81-2.73 (m, 1H), 2.51-2.44 (m, 1H), 2.34 (s, 3H). LC/MS (ESI) m/z: 608 (M − H)⁺. RT (Method A): 1.34 min. |
| 312^{a} | 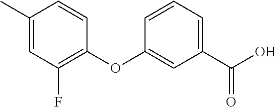 | ¹H NMR (400 MHz, CD₃OD): δ 8.69 (s, 1H), 7.92 (d, J = 5.9 Hz, 1H), 7.62 (d, J = 7.7 Hz, 1H), 7.47 (d, J = 8.1 Hz, 2H), 7.14 (d, J = 5.8 Hz, 2H), 7.07-7.01 (m, 2H), 6.99 (d, J = 8.1 Hz, 1H), 6.63 (t, J = 58.9 Hz, 1H), 6.43 (d, J = 74.5 Hz, 1H), 5.32- 5.27 (m, 1H), 5.05 (s, 1H), 4.58-4.53 (m, 1H), 4.31 (d, J = 16.7 Hz, 1H), 4.17-4.12 (m, 1H), 3.98-3.93 (m, 1H), 3.88 (d, J = 11.4 Hz, 1H), 2.50-2.44 (m, 1H), 2.33 (s, 3H), 2.30-2.25 (m, 1H), 1.59 (d, J = 7.0 Hz, 3H). LC/MS (ESI) m/z: 610 (M + H)⁺. RT (Method A): 1.58 min. |
| 315 | 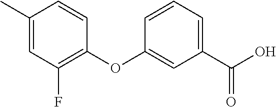 | ¹H NMR (400 MHz, CD₃OD): δ 8.65 (d, J = 0.6 Hz, 1H), 8.00 (d, J = 5.9 Hz, 1H), 7.54 (dd, J = 7.9, 1.2 Hz, 1H), 7.42-7.37 (m, 2H), 7.23 (d, J = 5.9 Hz, 1H), 7.11- 7.03 (m, 2H), 7.02-6.96 (m, 2H), 6.49 (d, J = 0.6 Hz, 1H), 4.82 (d, J = 3.8 Hz, 1H), 4.64 (dd, J = 15.9, 0.4 Hz, 1H), 4.43 (dd, J = 25.0, 16.2 Hz, 2H), 4.29 (d, J = 16.5 Hz, 1H), 3.38 (dd, J = 6.0, 2.5 Hz, 1H), 2.45-2.38 (m, 1H), 2.33 (s, 3H), 2.20 (dd, J = 13.4, 3.7 Hz, 1H), 1.30 (s, 3H), 1.26-1.21 (m, 1H), 0.81 (td, J = 5.8, 1.1 Hz, 1H). LC/MS (ESI) m/z: 556 (M + H)⁺. |

^{^{a}}HCl/1,4-dioxane was used in place of TFA in Step 4.

### Scheme 95. Synthesis of (2S,4R)-N-((1H-pyrrolo[3,2-c]pyridin-2-yl)methyl)-4-(difluoromethoxy)-1-((4-(4-fluoro-2-methylphenoxy)benzoyl)glycyl)pyrrolidine-2-carboxamide (Compound 308)

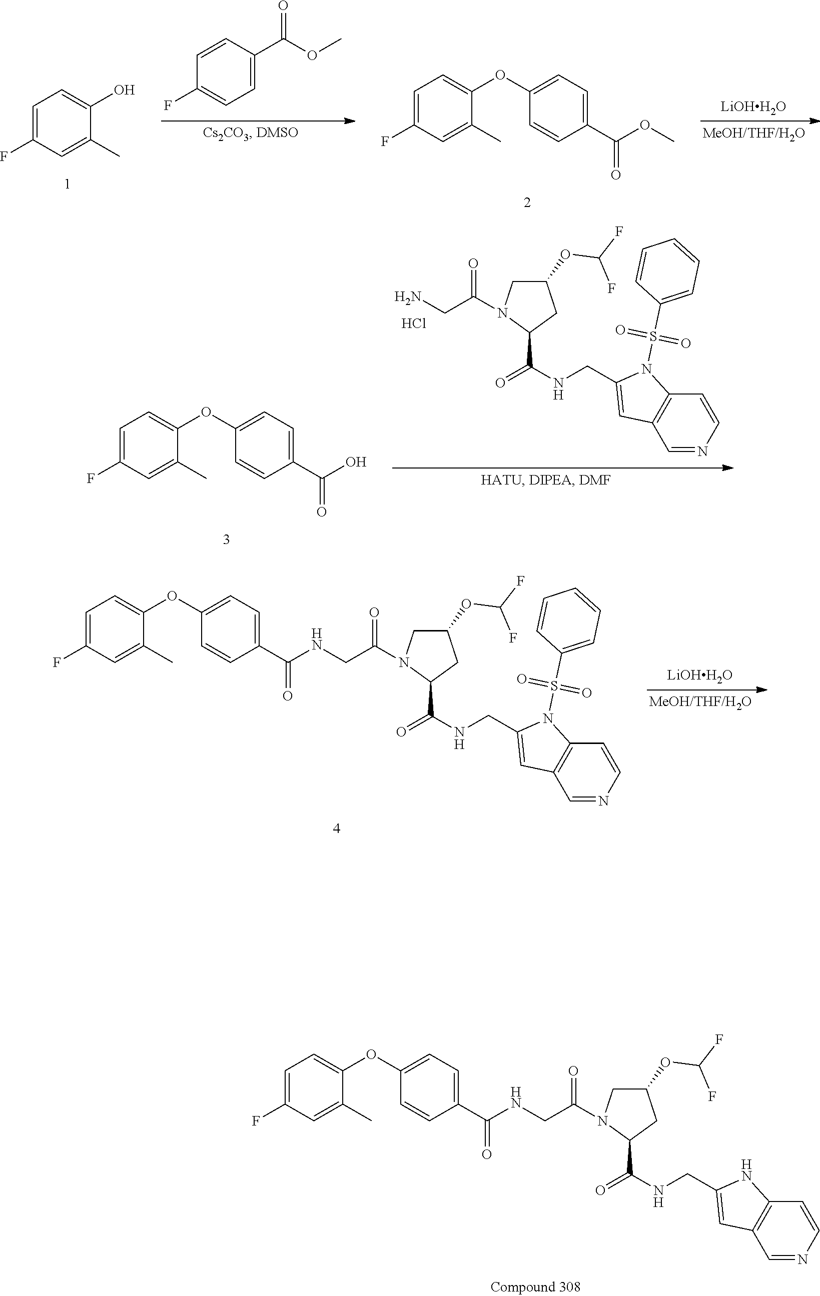

### Step 1: Methyl 4-(4-fluoro-2-methylphenoxy)benzoate (2)

To a mixture of 4-fluoro-2-methylphenol (1.0 g, 7.93 mmol) and methyl 4-fluorobenzoate (1.2 g, 7.93 mmol) in DMSO (10 mL) was added Cs₂CO₃ (7.7 g, 23.8 mmol) under N₂ atmosphere and the mixture was stirred under N₂ atmosphere at 100° C. overnight. The reaction mixture was diluted with EtOAc, washed with water and brine, dried over anhydrous Na₂SO₄, filtered, and concentrated under reduced pressure to dryness. The residue was purified by flash chromatography (silica gel, 0-20% EtOAc in PE) to give the title compound (682 mg, yield 70.4%) as a yellow oil. LC/MS (ESI) m/z: 261 (M+H)⁺.

### Step 2: 4-(4-Fluoro-2-methylphenoxy)benzoic acid (3)

To a solution of methyl 4-(4-fluoro-2-methylphenoxy)benzoate (682 mg, 2.62 mmol) in MeOH/THF/H₂O (6 mL, v/v/v=4/1/1) was added LiOH·H₂O (330 mg, 7.87 mmol) and the reaction mixture was stirred at room temperature for 1 hour. The mixture was acidified with 1N aq·HCl to pH˜3 and extracted with EtOAc twice. The combined organic layers were dried over anhydrous Na₂SO₄, filtered, and concentrated under reduced pressure to give the title compound (625 mg, yield 96.8%) as a white solid, which was used directly in the next reaction without further purification. LC/MS (ESI) m/z: 247 (M+H)⁺.

### Step 3: (2S,4R)-4-(difluoromethoxy)-1-((4-(4-fluoro-2-methylphenoxy)benzoyl) glycyl)-N-((1-(phenylsulfonyl)-H-pyrrolo[3,2-c]pyridin-2-yl)methyl)pyrrolidine-2-carboxamide (4)

To a mixture of 4-(4-fluoro-2-methylphenoxy)benzoic acid (14 mg, 0.06 mmol) and (2S,4R)-4-(difluoromethoxy)-1-glycyl-N-((1-(phenylsulfonyl)-1H-pyrrolo[3,2-c]pyridin-2-yl)methyl)pyrrolidine-2-carboxamide hydrochloride (30 mg, 0.06 mmol) in DMF (5 mL) was added DIPEA (38 mg, 0.29 mmol) and HATU (27 mg, 0.07 mmol) under N₂ atmosphere and the reaction mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=15:1) to give the title compound (20 mg, yield 11.8%) as a white solid. LC/MS (ESI) m/z: 736 (M+H)⁺.

### Step 4: (2S,4R)-N-((1H-pyrrolo[3, 2-c]pyridin-2-yl)methyl)-4-(difluoromethoxy)-1-((4-(4-fluoro-2-methylphenoxy)benzoyl)glycyl)pyrrolidine-2-carboxamide (Compound 308)

To a solution of (2S,4R)-4-(difluoromethoxy)-1-((4-(4-fluoro-2-methylphenoxy)benzoyl) glycyl)-N-((1-(phenylsulfonyl)-III-pyrrolo[3,2-c]pyridin-2-yl)methyl)pyrrolidine-2-carboxamide (20 mg, 0.03 mmol) in MeOII/TIIF/H₂O (2 mL, v/v/v=4/1/1) was added LiOH H₂ (10 mg, 0.16 mmol) under N₂ atmosphere and the reaction mixture was stirred at room temperature for 2 hours. The reaction mixture was concentrated under reduced pressure to dryness. The residue was purified by prep-HPLC to give the title compound (1.1 mg, yield 6.79%) as a white solid. ¹H NMR (400 MHz, CD₃OD): δ 8.63 (d, J=0.6 Hz, 1H), 7.97 (d, J=5.8 Hz, 1H), 7.85-7.82 (m, 2H), 7.18 (d, J=5.8 Hz, 1H), 7.11-7.08 (m, 1H), 7.02-6.99 (m, 2H), 6.89-6.86 (m, 2H), 6.71-6.33 (m, 2H), 5.06-5.02 (m, 1H), 4.71-4.66 (m, 1H), 4.60-4.51 (m, 2H), 4.21 (dt, J=16.7, 8.4 Hz, 2H), 4.00-3.95 (m, 1H), 3.91-3.87 (m, 1H), 2.52-2.45 (m, 1H), 2.31-2.24 (m, 1H), 2.17 (s, 3H). LC/MS (ESI) m/z: 596 (M+H)⁺. RT (Method A): 1.56 min.

The following compounds were prepared based on Scheme 95:

| Cmpd # | Reactant A | Reactant B | Reactant C |
|---|---|---|---|
| 316 | 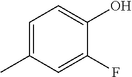 | 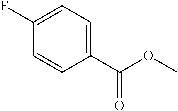 | 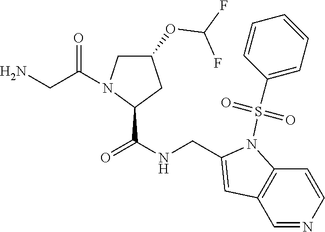 [SMILES: NCC(=O)N1C[C@H](OC(F)F)C[C@H]1C(=O)NCc1cc2cnccc2n1S(=O)(=O)c1ccccc1] |
| 323 | 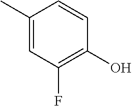 | 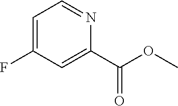 | 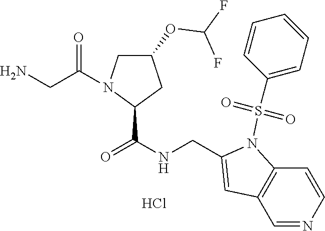 [SMILES: NCC(=O)N1C[C@H](OC(F)F)C[C@H]1C(=O)NCc1cc2cnccc2n1S(=O)(=O)c1ccccc1 ; Cl] |

| Cmpd # | Characterization Data |
|---|---|
| 316 | ¹H NMR (400 MHz, CD₃OD): δ 8.76 (s, 1H), 8.03 (d, J = 6.1 Hz, |
| | 1H), 7.82 (d, J = 8.9 Hz, 2H), 7.37 (d, J = 6.5 Hz, 1H), 7.11 (d, J = |
| | 8.2 Hz, 1H), 7.07 (d, J = 3.6 Hz, 1H), 6.93 (d, J = 8.8 Hz, 2H), 6.71- |
| | 6.33 (m, 2H), 5.34 (s, 1H), 4.22 (s, 1H), 4.21 (s, 1H), 4.01 (d, J = |
| | 3.9 Hz, 1H), 3.98 (d, J = 4.5 Hz, 1H), 3.96 (s, 1H), 2.48 (d, J = 9.9 |
| | Hz, 1H), 2.38 (s, 3H), 2.29 (dd, J = 8.4, 4.8 Hz, 1H), 2.21-2.16 (m, |
| | 2H). LC/MS (ESI) m/z: 596 (M + H)⁺. RT (Method A): 1.56 min. |
| 323 | ¹H NMR (400 MHz, CD₃OD): δ 8.85 (s, 1H), 8.55 (d, J = 5.6 Hz, |
| | 1H), 8.12 (d, J = 6.4 Hz, 1H), 7.59 (d, J = 6.4 Hz, 1H), 7.50 (d, J = |
| | 2.6 Hz, 1H), 7.15 (d, J = 8.2 Hz, 2H), 7.11 (d, J = 2.4 Hz, 1H), 7.10 |
| | (d, J = 2.5 Hz, 1H), 6.77 (s, 1H), 6.52 (t, J = 74.4 Hz, 1H), 5.06- |
| | 5.03 (m, 1H), 4.79-4.75 (m, 1H), 4.58 (t, J = 3.9 Hz, 2H), 4.29 (d, |
| | J = 10.3 Hz, 2H), 3.99-3.95 (m, 1H), 3.89-3.85 (m, 1H), 2.52- |
| | 2.47 (m, 1H), 2.39 (s, 3H), 2.31-2.25 (m, 1H). LC/MS (ESI) m/z: |
| | 597 (M + H)⁺. RT (Method A): 1.53 min. |

### Scheme 96. Synthesis of (S)-N-((1H-pyrrolo[3,2-c]pyridin-2-yl)methyl)-1-((3-(2-fluoro-4-methylphenoxy)benzoyl)glycyl)pyrrolidine-2-carboxamide (Compound 310)

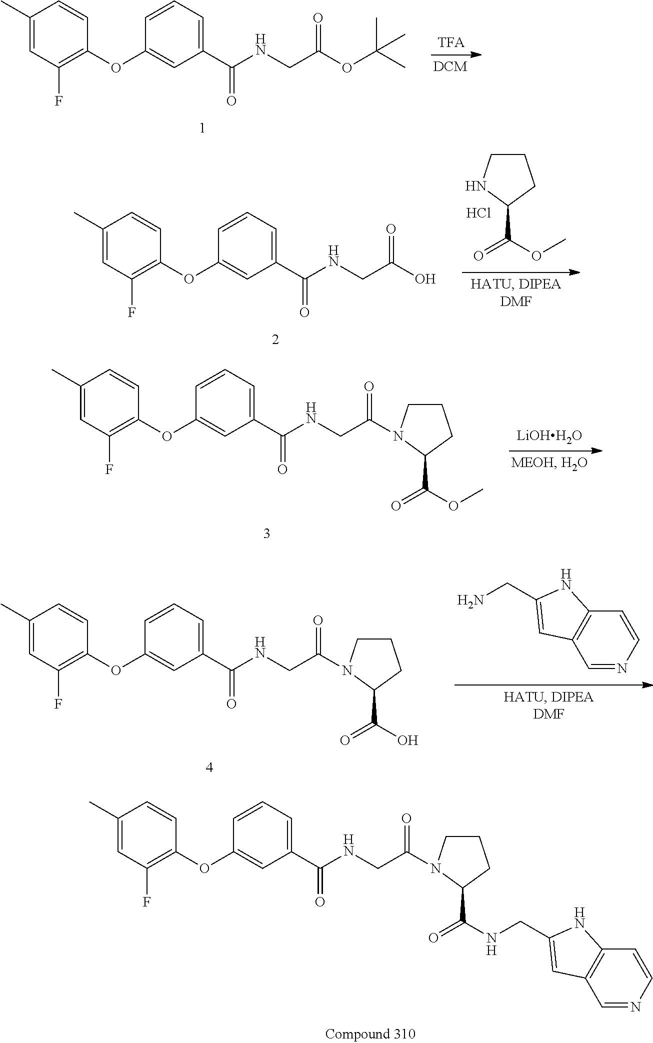

### Step 1: (3-(2-Fluoro-4-methylphenoxy)benzoyl)glycine (2)

To a solution of tert-butyl (3-(2-fluoro-4-methylphenoxy)benzoyl)glycinate (105 mg, 0.42 mmol) in DCM (2 mL) was added TFA (2 mL) and the mixture was stirred at room temperature for 1 hour. The mixture was concentrated under reduced pressure to dryness to give the title compound (126 mg, yield 100%) as a colorless oil, which was used directly in the next step without further purification. LC/MS (ESI) m/z: 304 (M+H)⁺.

### Step 2: Methyl (3-(2-fluoro-4-methylphenoxy)benzoyl)glycyl-L-prolinate (3)

To a mixture of (3-(2-fluoro-4-methylphenoxy)benzoyl)glycine (126 mg, 0.42 mmol) and methyl L-prolinate hydrochloride (104 mg, 0.63 mmol) in DMF (3 mL) was added DIPEA (269 mg, 2.08 mmol) and HATU (238 mg, 0.63 mmol) under N₂ atmosphere and the reaction mixture was stirred at room temperature for 20 minutes. The mixture was diluted with EtOAc, washed with saturated aq·NH₄Cl solution and brine, dried over anhydrous Na₂SO₄, filtered, and concentrated under reduced pressure to dryness. The residue was purified by flash chromatography (silica gel, 0-15% EtOAc in PE) to give the title compound (167 mg, yield 96.7%) as a colorless oil. LC/MS (ESI) m/z: 415 (M+H)⁺.

### Step 3: (3-(2-Fluoro-4-methylphenoxy)benzoyl)glycyl-L-prolinate (4)

To a solution of methyl (3-(2-fluoro-4-methylphenoxy)benzoyl)glycyl-L-prolinate (167 mg, 0.40 mmol) in MeOH/H₂O (5 mL, v/v=2/1) was added LiOH H₂O (68 mg, 1.62 mmol) and the mixture was stirred at room temperature for 1 hour. The mixture was acidified with 1N aq·HCl to pH˜5 and extracted with EtOAc twice. The combined organic layers were washed with brine, dried over anhydrous Na₂SO₄, filtered, and concentrated under reduced pressure to dryness to give the title compound (119 mg, yield 73.7%) as a colorless oil, which was used directly in the next step without further purification. LC/MS (ESI) m/z: 401 (M+H)⁺.

### Step 4: (S)-N-((1H-pyrrolo[3,2-c]pyridin-2-yl)methyl)-1-((3-(2-fluoro-4-methylphenoxy)benzoyl)glycyl)pyrrolidine-2-carboxamide (Compound 310)

To a mixture of (3-(2-fluoro-4-methylphenoxy)benzoyl)glycyl-L-proline (30 mg, 0.08 mmol) and (1H-pyrrolo[3,2-c]pyridin-2-yl)methanamine (17 mg, 0.11 mmol) in DMF (1 mL) was added DIPEA (48 mg, 0.37 mmol) and HATU (43 mg, 0.11 mmol) under N₂ atmosphere and the reaction mixture was stirred at room temperature for 1 hour. The mixture was diluted with EtOAc, washed with saturated aq·NH₄Cl solution and brine, dried over anhydrous Na₂SO₄, filtered, and concentrated under reduced pressure to dryness. The residue was purified by prep-TLC (DCM:MeOH=8:1) and further purified by prep-HPLC to give Compound 310 (2.3 mg, yield 5.8%) as a colorless oil. 11¹H NMR (400 MHz, CD₃OD): δ 8.65 (s, 1H), 8.01 (d, J=5.8 Hz, 1H), 7.54 (d, J=7.9 Hz, 1H), 7.43-7.39 (m, 2H), 7.26 (d, J=5.7 Hz, 1H), 7.11-7.05 (m, 2H), 7.03-6.98 (m, 2H), 6.52 (s, 1H), 4.66-4.45 (m, 4H), 4.21 (s, 2H), 3.80-3.67 (m, 2H), 2.34 (s, 3H), 2.22-2.16 (m, 1H), 2.08-2.04 (m, 2H). LC/MS (ESI) m/z: 530 (M+H)⁺. RT (Method A): 1.36 min.

### Scheme 97. Synthesis of (2S,4R)-N-((1H-pyrrolo[3,2-c]pyridin-2-yl)methyl)-4-(difluoromethoxy)-1-((5-(2-fluoro-4-methylphenoxy)-2-methoxybenzoyl)glycyl)pyrrolidine-2-carboxamide (Compound 3H)

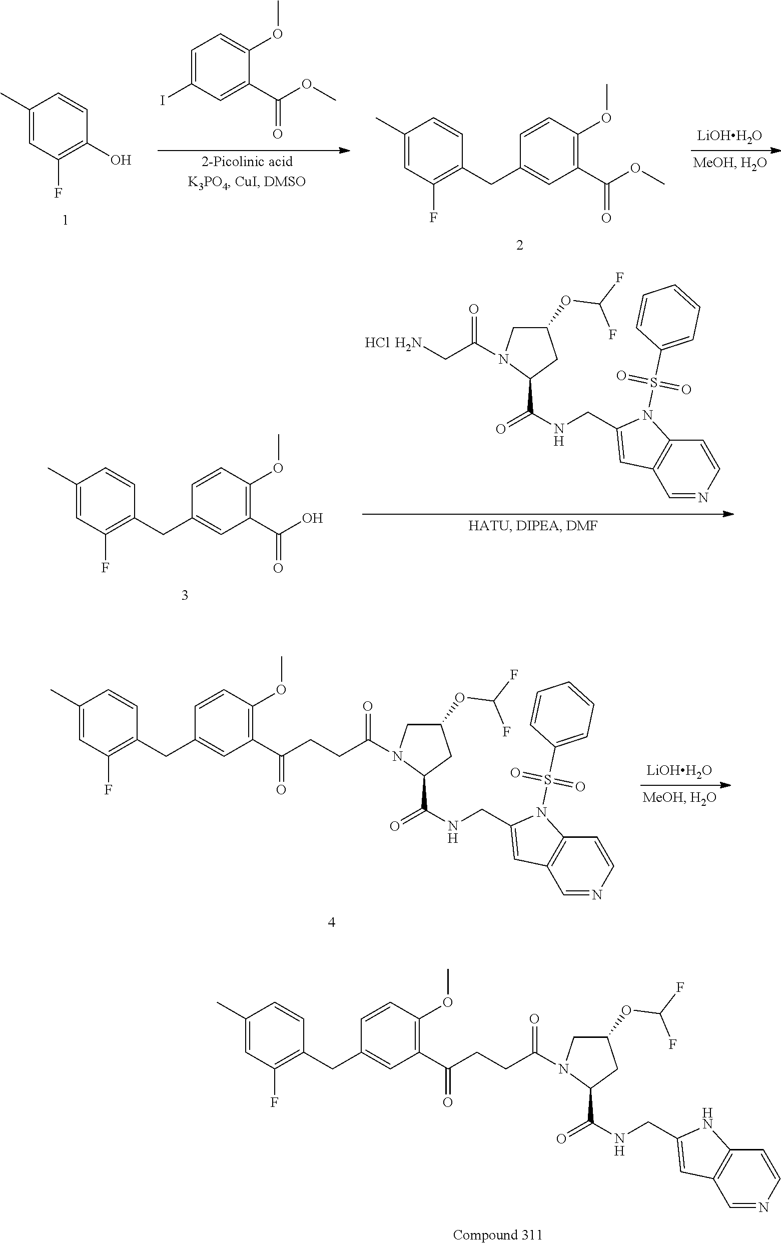

### Step 1: Methyl 5-(2-fluoro-4-methylphenoxy)-2-methoxybenzoate (2)

To a mixture of 2-fluoro-4-methylphenol (100 mg, 0.79 mmol) and methyl 5-iodo-2-methoxybenzoate (200 mg, 0.68 mmol) in DMSO (1 mL) was added 2-Picolinic acid (8 mg, 0.065 mmol), K₃PO₄ (290 mg, 1.36 mmol) and CuI (6 mg, 0.032 mmol) under N₂ atmosphere, the mixture was degassed under N₂ atmosphere for three times and stirred at 80° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-10% EtOAc in PE) to give the title compound (90 mg, yield 45.2%) as a colorless oil. LC/MS (ESI) m/z: 291 (M+H)⁺.

### Step 2: 5-(2-Fluoro-4-methylphenoxy)-2-methoxybenzoic acid (3)

To a solution of methyl 5-(2-fluoro-4-methylphenoxy)-2-methoxybenzoate (90 mg, 0.31 mmol) in MeOH/water (1.5 mL, v/v=4/1) was added LiOH·H₂O (26 mg, 0.62 mmol) under N₂ atmosphere and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (70 mg, yield 81.7%) as a yellow solid, which was used directly in the next step without further purification. LC/MS (ESI) m/z: 277 (M+H)⁺.

### Step 3: (2S,4R)-4-(difluoromethoxy)-1-((5-(2-fluoro-4-methylphenoxy)-2-methoxybenzoyl)glycyl)-N-((1-(phenylsulfonyl)-1H-pyrrolo[3,2-c]pyridin-2-yl)methyl)pyrrolidine-2-carboxamide

To a mixture of 5-(2-fluoro-4-methylphenoxy)-2-methoxybenzoic acid (70 mg, 0.25 mmol) and (2S,4R)-4-(difluoromethoxy)-1-glycyl-N-((1-(phenylsulfonyl)-1H-pyrrolo[3,2-c]pyridin-2-yl)methyl)pyrrolidine-2-carboxamide hydrochloride (100 mg, 0.20 mmol) in DMF (2 mL) was added DIPEA (164 mg, 1.27 mmol) and HATU (116 mg, 0.31 mmol) under N₂ atmosphere and the mixture was stirred at room temperature for 2 hours. The mixture was diluted with EtOAc, washed with saturated aq·NH₄Cl solution and brine, dried over anhydrous Na₂SO₄, filtered, and concentrated under reduced pressure to dryness. The residue was purified by prep-TLC (DCM:MeOH=10:1) to give the title compound (30 mg, yield 14.0%) as a yellow solid. LC/MS (ESI) m/z: 766 (M+H)⁺.

### Step 4: (2S,4R)-N-((1H-pyrrolo[3, 2-c]pyridin-2-yl)methyl)-4-(difluoromethoxy)-1-((5-(2-fluoro-4-methylphenoxy)-2-methoxybenzoyl)glycyl)pyrrolidine-2-carboxamide (Compound 3H)

To a solution of (2S,4R)-4-(difluoromethoxy)-1-((5-(2-fluoro-4-methylphenoxy)-2-methoxybenzoyl)glycyl)-N-((1-(phenylsulfonyl)-1H-pyrrolo[3,2-c]pyridin-2-yl)methyl)pyrrolidine-2-carboxamide (30 mg, 0.036 mmol) in MeOH/water (1.5 mL, v/v=4/1) was added LiOH·H₂O (3 mg, 0.072 mmol) and the reaction mixture was stirred at room temperature for 2 hours. The mixture was concentrated under reduced pressure to dryness. The residue was purified by prep-HPLC to give Compound 311 (1.0 mg, yield 4.5%) as a white solid. ¹H NMR (400 MHz, CD₃OD): δ 8.65 (s, 1H), 8.01 (d, J=5.9 Hz, 1H), 7.56 (d, J=2.3 Hz, 1H), 7.26 (d, J=5.9 Hz, 1H), 7.15 (d, J=2.7 Hz. 2H), 7.03 (d, J=12.2 Hz, 1H), 6.94 (s, 1H), 6.54 (s, 1H), 6.43 (t, 1H), 5.02 (s, 1H), 4.66-4.64 (m, 1H), 4.60-4.58 (m, 1H), 4.56-4.54 (m, 1H), 4.30-4.27 (m, 1H), 4.27-4.24 (m, 1H), 4.01-3.97 (m, 1H), 3.96 (s, 3H), 3.85-3.81 (m, 1H), 2.52-2.46 (m, 1H), 2.32 (s, 3H), 2.29-2.24 (m, 1H). LC/MS (ESI) m/z: 626 (M+H)⁺. RT (Method A): 1.64 min.

### Scheme 98. Synthesis of (2S,4R)-N-((1H-pyrrolo[3,2-c]pyridin-2-yl)methyl)-4-(difluoromethoxy)-1-((9-hydroxy-9-methyl-9H-fluorene-3-carbonyl)glycyl) pyrrolidine-2-carboxamide (Compound 314)

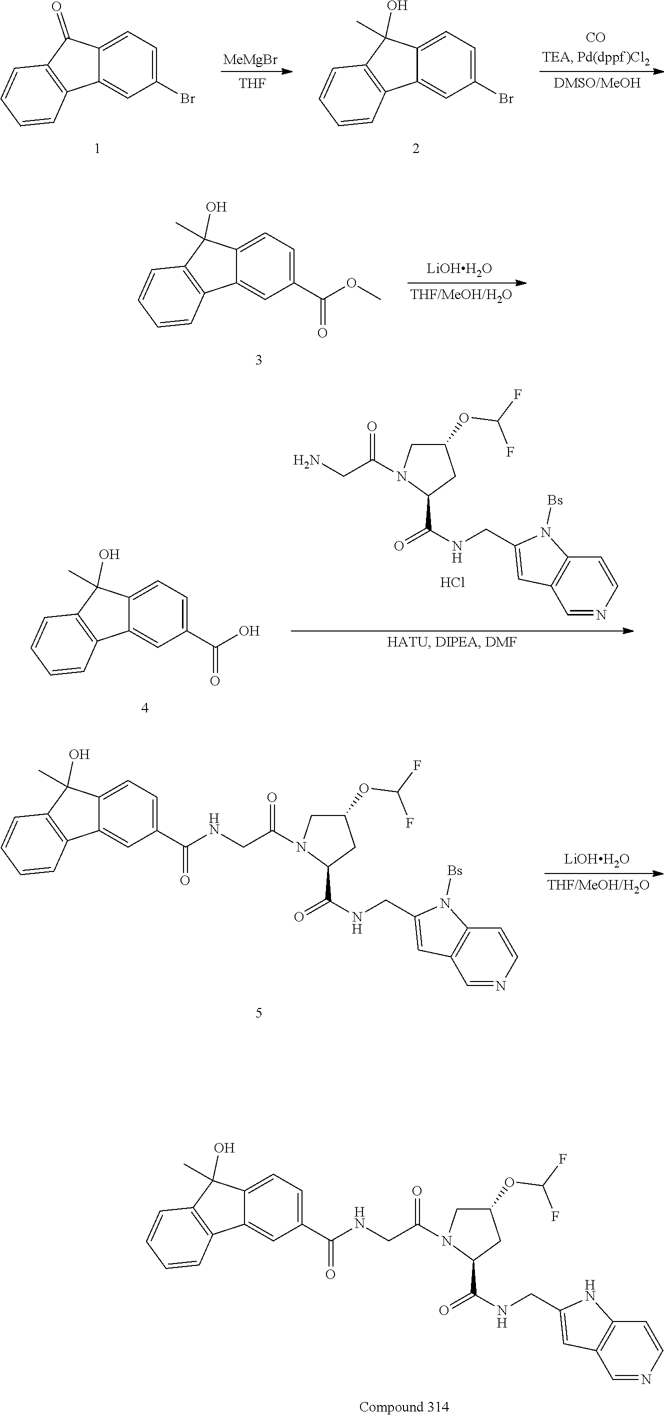

### Step 1: 3-Bromo-9-methyl-9H-fluoren-9-ol (2)

To a solution of 3-bromo-9H-fluoren-9-one (500 mg, 1.93 mmol) in THF (10 mL) was added CH₃MgBr (6.4 mL, 3.0M) under N₂ atmosphere and the reaction mixture was stirred at room temperature for 2 hours. The reaction mixture was quenched with saturated aq·NH₄Cl solution and extracted with EtOAc twice. The combined organic layers were dried over anhydrous Na₂SO₄, filtered, and concentrated under reduced pressure to dryness. The residue was purified by flash chromatography (silica gel, 0-20% EtOAc in PE) to give the title compound (520 mg, yield 97.9%) as a white solid. LC/MS (ESI) m/z: 257 (M+H)⁺.

### Step 2: Methyl 9-hydroxy-9-methyl-9H-fluorene-3-carboxylate (3)

To a solution of 3-bromo-9-methyl-9H-fluoren-9-ol (520 mg, 1.89 mmol) in MeOH (3 mL) and DMSO (3 mL) was added TEA (574 mg, 5.67 mmol), Pd(dppf)C₁₂ (277 mg, 0.38 mmol), the mixture was degassed under N₂ atmosphere for three times and stirred under a CO balloon at 80° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-30% EtOAc in PE) to give the title compound (380 mg, yield 79.1%) as a white solid. LC/MS (ESI) m/z: 237 (M+H)⁺.

### Step 3: 9-Hydroxy-9-methyl-9H-fluorene-3-carboxylic acid (4)

To a solution of methyl 9-hydroxy-9-methyl-9H-fluorene-3-carboxylate (380 mg, 1.49 mmol) in THF/MeOH/H₂O (6 mL, v/v/v=4/1/1) was added LiOH·H₂O (125 mg, 2.99 mmol) under N₂ atmosphere and the reaction mixture was stirred at room temperature for 2 hours. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (355 mg, yield 98.9%) as a white solid, which was used directly in the next step without further purification. LC/MS (ESI) m/z: 223 (M+H)⁺.

### Step 4: (2S,4R)-4-(difluoromethoxy)-1-((9-hydroxy-9-methyl-9H-fluorene-3-carbonyl) glycyl)-N-((1-(phenylsulfonyl)-H-pyrrolo[3,2-c]pyridin-2-yl)methyl)pyrrolidine-2-carboxamide (5)

To a mixture of 9-hydroxy-9-methyl-9H-fluorene-3-carboxylic acid (30 mg, 0.12 mmol) and (2S,4R)-4-(difluoromethoxy)-1-glycyl-N-((1-(phenylsulfonyl)-1H-pyrrolo[3,2-c]pyridin-2-yl)methyl)pyrrolidine-2-carboxamide hydrochloride (82 mg, 0.15 mmol) in DMF (5 mL) was added HATU (52 mg, 0.14 mmol) and DIPEA (97 mg, 0.75 mmol) under N₂ atmosphere and the reaction mixture was stirred at room temperature for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-5% MeOH in DCM) to give the title compound (30 mg, yield 33.0%) as a white solid. LC/MS (ESI) m/z: 730 (M+H)⁺.

### Step 5: (2S,4R)-N-((1H-pyrrolo[3, 2-c]pyridin-2-yl)methyl)-4-(difluoromethoxy)-1-((9-hydroxy-9-methyl-9H-fluorene-3-carbonyl)glycyl)pyrrolidine-2-carboxamide (Compound 314)

To a solution of (2S,4R)-4-(difluoromethoxy)-1-((9-hydroxy-9-methyl-9H-fluorene-3-carbonyl)glycyl)-N-((1-(phenylsulfonyl)-1H-pyrrolo[3,2-c]pyridin-2-yl)methyl) pyrrolidine-2-carboxamide (30 mg, 0.041 mmol) in THF/MeOH/H₂O (3 mL, v/v/v=4/1/1) was added LiOH H₂O (10 mg, 0.25 mmol) under N₂ atmosphere and the reaction mixture was stirred at room temperature for 2 hours. The mixture was concentrated under reduced pressure to dryness. The residue was purified by prep-HPLC to give Compound 314 (8 mg, yield 33.0%) as a white solid. ¹H NMR (400 MHz, CD₃OD): δ 8.70-8.54 (m, 1H), 8.18-8.06 (m, 1H), 7.84 (m, J=38.7, 18.2, 5.1 Hz, 2H), 7.72-7.55 (m, SLXNHB3H), 4.38-4.13 (m, 2H), 4.06-3.88 (m, 21H), 2.59-2.43 (m, 1H), 2.33-2.26 (m, J=34.3, 19.5, 15.4 Hz, 1H), 1.70 (s, 3H). LC/MS (ESI) m/z: 590 (M+H)⁺. RT (Method A): 1.08 min.

### Scheme 99. Synthesis of (2S,4R)-N-((1H-pyrrolo[3,2-c]pyridin-2-yl)methyl)-4-(difluoromethoxy)-1-((3-((2-fluoro-4-methylbenzyl)oxy)benzoyl)glycyl)pyrrolidine-2-carboxamide (Compound 325)

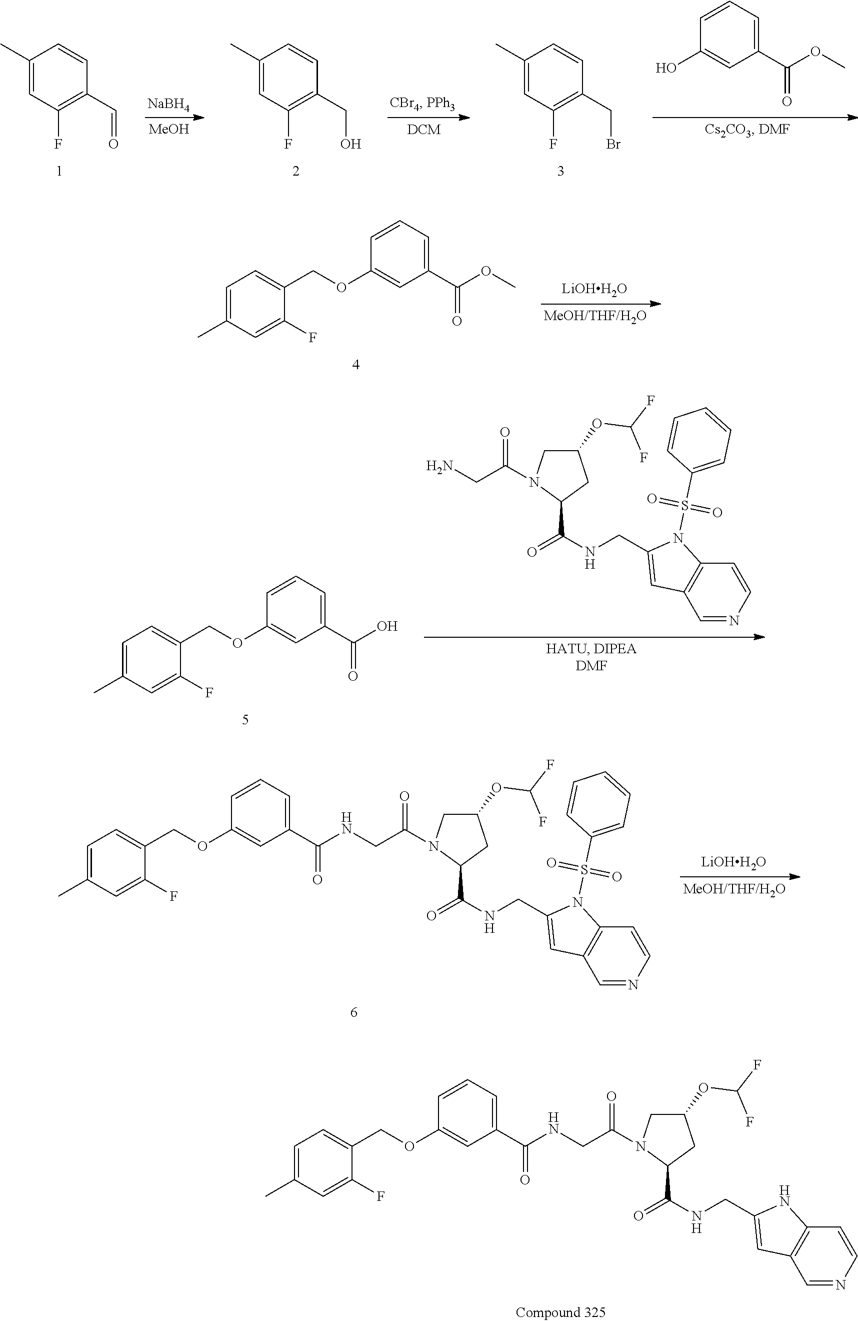

### Step 1: (2-Fluoro-4-methylphenyl)methanol (2)

To a solution of 2-fluoro-4-methylbenzaldehyde (1.0 g, 7.2 mmol) in MeOH (10 mL) was added NaBH₄ (826 mg, 21.7 mmol) in portions at 0° C. under N₂ atmosphere, the reaction mixture was degassed under N₂ atmosphere for three times and stirred at room temperature for 2 hours. The mixture was quenched with saturated aq·NH₄Cl solution and extracted with EtOAc twice. The combined organic layers were washed with brine, dried over anhydrous Na₂SO₄, filtered, and concentrated under reduced pressure to dryness to give the title compound (1.0 g, yield 99.0%) as a colorless oil. ¹H NMR (400 MHz, CDCl₃) δ 7.42 (t, J=7.8 Hz, 1H), 7.10 (d, J=7.7 Hz, 1H), 7.02 (d, =11.1 Hz, 1H), 4.84 (s, 2H), 2.50 (s, 3H).

### Step 2: 1-(Bromomethyl)-2-fluoro-4-methylbenzene (3)

To a solution of (2-fluoro-4-methylphenyl)methanol (500 mg, 3.6 mmol) in DCM (8 mL) was added CBr₄ (2.4 g, 7.1 mmol) and PPh₃ (1.9 g, 7.1 mmol) at 0° C. under N₂ atmosphere and the reaction mixture was stirred under N₂ atmosphere at room temperature for 2 hours. The mixture was concentrated to dryness to give crude title compound (2.5 g, crude) as a yellow solid, which was directly used in the next reaction without purification.

### Step 3: Methyl 3-((2-fluoro-4-methylbenzyl)oxy)benzoate (4)

To a mixture of 1-(bromomethyl)-2-fluoro-4-methylbenzene (2.5 g, 3.6 mmol) and methyl 3-hydroxybenzoate (376 mg, 2.5 mmol) in DMF (8 mL) was added Cs₂CO₃ (2.9 g, 7.5 mmol) at 25° C. and the reaction mixture was stirred under N₂ atmosphere at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (460 mg, yield 67.8%) as a colorless oil. LC/MS (ESI) m/z: 275 (M+H)⁺.

### Step 4: 3-((2-fluoro-4-methylbenzyl)oxy)benzoic acid (5)

To a solution of methyl 3-((2-fluoro-4-methylbenzyl)oxy)benzoate (450 mg, 1.6 mmol) in THF/MeOH/water (8 mL, v/v/v=2/1/1) was added LiOH 1120 (207 mg, 4.9 mmol) and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (410 mg, yield 96.0%) as a white solid, which was used directly in the next step without further purification. LC/MS (ESI) m/z: 259 (M−H)⁺.

### Step 5: (2S,4R)-4-(difluoromethoxy)-1-((3-((2-fluoro-4-methylbenzyl)oxy)benzoyl) glycyl)-N-((1-(phenylsulfonyl)-1H-pyrrolo[3,2-c]pyridin-2-yl)methyl)pyrrolidine-2-carboxamide (6)

To a mixture of 3-((2-fluoro-4-methylbenzyl)oxy)benzoic acid (20 mg, 0.08 mmol) and (2S,4R)-4-(difluoromethoxy)-1-glycyl-N-((1-(phenylsulfonyl)-1H-pyrrolo[3,2-c]pyridin-2-yl)methyl)pyrrolidine-2-carboxamide (47 mg, 0.09 mmol) in DMF (2 mL) was added DIPEA (50 mg, 0.38 mmol) and HATU (44 mg, 0.12 mmol) under N₂ atmosphere and the reaction mixture was stirred at room temperature for 2 hours. The mixture was filtered, and the filtrate was concentrated under reduced pressure to dryness. The residue was purified by prep-TLC (DCM:MeOH=15:1) to give the title compound (20 mg, yield 34.7%) as a white solid. LC/MS (ESI) m/z: 750 (M+H)⁺.

### Step 6: (2S,4R)-N-((1H-pyrrolo[3, 2-c]pyridin-2-yl)methyl)-4-(difluoromethoxy)-1-((3-((2-fluoro-4-methylbenzyl)oxy)benzoyl)glycyl)pyrrolidine-2-carboxamide (Compound 325)

To a solution of (2S,4R)-4-(difluoromethoxy)-1-((3-((2-fluoro-4-methylbenzyl)oxy) benzoyl)glycyl)-N-((1-(phenylsulfonyl)-1H-pyrrolo[3,2-c]pyridin-2-yl)methyl) pyrrolidine-2-carboxamide (20 mg, 0.03 mmol) in THF/MeOH/water (2 mL, v/v/v=2/1/1) was added LiOH·H₂O (6 mug, 0.13 mmol) under N₂ atmosphere and the reaction mixture was stirred at room temperature for 2 hours. The mixture was concentrated under reduced pressure to dryness. The residue was purified by prep-HPLC to give Compound 325 (1.5 mg, yield 9.2%) as a white solid. ¹H NMR (400 MHz, CD₃OD): δ 8.79 (s, 1H), 8.03 (d, J=6.2 Hz, 1H), 7.50-7.44 (m, 3H), 7.37 (d, J=7.5 Hz, 2H), 7.19 (d, J=7.6 Hz, 1H), 7.01-6.94 (m, 2H), 6.79 (s, 1H), 6.53 (t, J=74.5 Hz, 1H), 5.10 (s, 2H), 5.05 (s, 1H), 4.71 (s, 1H), 4.63 (d, J=5.9 Hz, 1H), 4.59 (d, J=7.3 Hz, 2H), 4.23 (s, 2H), 4.04-3.99 (m, 1H), 3.91 (d, J=11.6 Hz, 1H), 2.54-2.48 (m, 1H), 2.34 (s, 3H), 2.32-2.25 (m, 1H). LC/MS (ESI) m/z: 610 (M+H)⁺. RT (Method A): 1.60 min.

### Scheme 100. Synthesis of (2S,4R)-N-((1H-pyrrolo[3,2-c]pyridin-2-yl)methyl)-4-(difluoromethoxy)-1-((3-fluoro-5-(2-fluoro-4-methylphenoxy)benzoyl)glycyl)pyrrolidine-2-carboxamide (Compound 329)

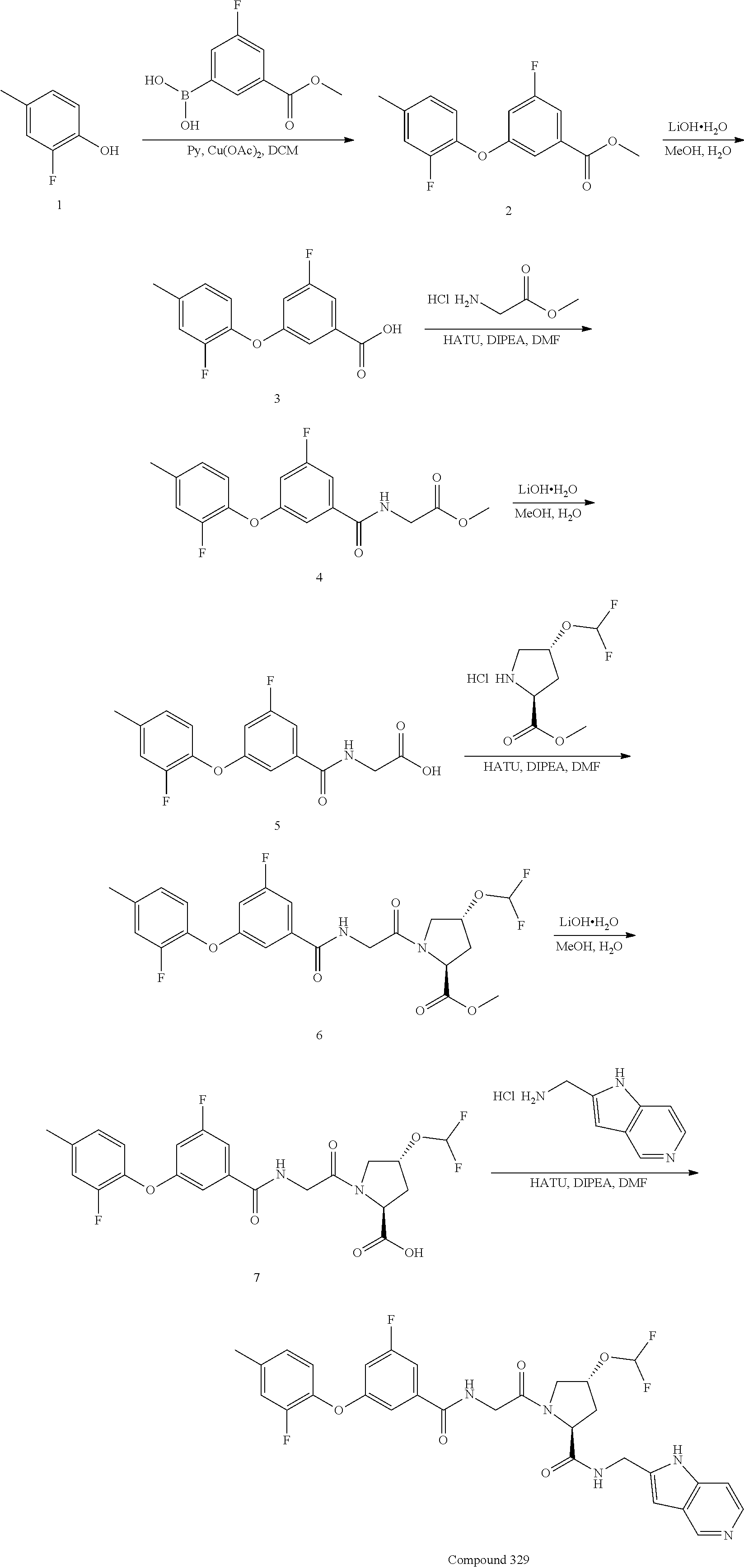

### Step 1: Methyl 3-fluoro-5-(2-fluoro-4-methylphenoxy)benzoate (2)

To a mixture of 2-fluoro-4-methylphenol (250 mg, 1.98 mmol) and (3-fluoro-5-(methoxycarbonyl)phenyl)boronic acid (787 mg, 3.97 mmol) in dry DCM (5 mL) was added pyridine (314 mg, 3.97 mmol), Cu(OAc)₂ (538 mg, 2.96 mmol) and the reaction mixture was stirred under 02 balloon at room temperature overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-7% EtOAc in PE) to give the title compound (420 mg, yield 76.7%) as a colorless oil. ¹H NMR (400 MHz, CDCl₃) δ 7.43-7.37 (m, 2H), 7.02 (dd, J=9.8, 6.0 Hz, 2H), 6.95 (d, J=8.5 Hz, 1H), 6.84 (d, J=9.5 Hz, 1H), 3.89 (d, J=0.6 Hz, 3H), 2.37 (s, 3H).

### Step 2: 3-Fluoro-5-(2-fluoro-4-methylphenoxy)benzoic acid (3)

To a solution of methyl 3-fluoro-5-(2-fluoro-4-methylphenoxy)benzoate (420 mg, 1.58 mmol) in MeOH/water (5 mL, v/v=4/1) was added LiOH·H₁₂O (133 mg, 3.17 mmol) under N₂ atmosphere and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (395 mg, yield 99.3%) as a yellow solid, which was used directly in the next step without further purification. LC/MS (ESI) m/z: 265 (M+H)⁺.

### Step 3: Methyl (3-fluoro-5-(2-fluoro-4-methylphenoxy)benzoyl)glycinate (4)

To a mixture of 3-fluoro-5-(2-fluoro-4-methylphenoxy)benzoic acid (395 mg, 1.51 mmol) and methyl glycinate hydrochloride (227 mg, 1.81 mmol) in DMF (4 mL) was added DIPEA (977 mg, 7.56 mmol) and HATU (691 mg, 1.81 mmol) under N₂ atmosphere and the mixture was stirred at room temperature for 1 hour. The mixture was diluted with EtOAc, washed with saturated aq·NaHCO₃ solution, dried over anhydrous Na₂SO₄, filtered, and concentrated under reduced pressure to dryness. The residue was purified by flash chromatography (silica gel, 0-50% EtOAc in PE) to give the title compound (430 mg, yield 84.7%) as a white solid. LC/MS (ESI) m/z: 336 (M+H)⁺.

### Step 4: (3-Fluoro-5-(2-fluoro-4-methylphenoxy)benzoyl)glycine (5)

To a solution of methyl (3-fluoro-5-(2-fluoro-4-methylphenoxy)benzoyl)glycinate (430 mg, 1.28 mol) in MeOH/water (5 mL, v/v=4/1) was added LiOH·H₂O (108 mg, 2.57 mmol) and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (400 mg, yield 97.0%) as a white solid, which was used directly in the next reaction without further purification. LC/MS (ESI) m/z: 322 (M+H)⁺.

### Step 5: Methyl (2S,4R)-4-(difluoromethoxy)-1-((3-fluoro-5-(2-fluoro-4-methylphenoxy)benzoyl)glycyl)pyrrolidine-2-carboxylate (6)

To a mixture of (3-fluoro-5-(2-fluoro-4-methylphenoxy)benzoyl)glycine (100 mg, 0.31 mmol) and methyl (2S,4R)-4-(difluoromethoxy)pyrrolidine-2-carboxylate hydrochloride (90 mg, 0.41 mmol) in DMF (1 mL) was added DIPEA (200 mg, 1.54 mmol) and HATU (142 mg, 0.37 mmol) under N₂ atmosphere and the mixture was stirred at room temperature for 1 hour. The mixture was diluted with EtOAc, washed with saturated aq·NaHCO₃ solution, dried over anhydrous Na₂SO₄, filtered, and concentrated under reduced pressure to dryness. The residue was purified by flash chromatography (silica gel, 0-50% EtOAc in PE) to give the title compound (135 mg, yield 87.0%) as a yellow oil. LC/MS (ESI) m/z: 499 (M+H)⁺.

### Step 6: (2S,4R)-4-(Difluoromethoxy)-1-((3-fluoro-5-(2-fluoro-4-methylphenoxy) benzoyl)glycyl)pyrrolidine-2-carboxylic acid (7)

To a solution of methyl (2S,4R)-4-(difluoromethoxy)-1-((3-fluoro-5-(2-fluoro-4-methylphenoxy)benzoyl)glycyl)pyrrolidine-2-carboxylate (135 mg, 0.27 mol) in MeOH/water (1.5 mL, v/v=4/1) was added LiOH·H₂O (26 mg, 0.62 mmol) under N₂ atmosphere and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (120 mg, yield 91.4%) as a white solid, which was used directly in the next step without further purification. LC/MS (ESI) m/z: 485 (M+H)⁺.

### Step 7: (2S,4R)-N-((1H-pyrrolo[3,2-c]pyridin-2-yl)methyl)-4-(difluoromethoxy)-1-((3-fluoro-5-(2-fluoro-4-methylphenoxy)benzoyl)glycyl)pyrrolidine-2-carboxamide (Compound 329)

To a mixture of (2S,4R)-4-(difluoromethoxy)-1-((3-fluoro-5-(2-fluoro-4-methylphenoxy) benzoyl)glycyl)pyrrolidine-2-carboxylic acid (40 mg, 0.082 mmol) and (1H-pyrrolo[3,2-c]pyridin-2-yl)methanamine hydrochloride (18 mg, 0.098 mmol) in DMF (1 mL) was added DIPEA (53 mg, 0.41 mmol) and HATU (38 mg, 0.10 mmol) under N₂ atmosphere and the mixture was stirred at roam temperature for 1 hour. The mixture was concentrated under reduced pressure to dryness. The residue was purified by prep-HPLC to give Compound 329 (5.9 mg, yield 11.6%) as a white solid. 7¹H NMR (400 MHz, CD₃OD): δ 8.64 (s, 1H), 7.99 (d, J=5.8 Hz, 1H), 7.31 (d, J=8.6 Hz, 1H), 7.25 (s, 1H) 7.21 (d, J=5.8 Hz, 1H), 7.08 (dd, J 14.2, 6.1 Hz, 2H), 7.02 (d, J=8.9 Hz, 1H), 6.84 (d, J=9.7 Hz, 1H), 6.60 (d, J=74.4 Hz, 1H), 6.41 (d, J=69.87 Hz, 1H), 5.03 (s, 1H),4.68 (d, J=16.2 Hz, 1H), 4.61-4.56 (m, 1H), 4.51 (d, J=15.9 Hz, 1H), 4.24 (d, J=6.6 Hz, 1H),4.15 (d, J=16.7 Hz, 1), 3.99-3.94 (m, 1H), 3.86 (d, J=11.9 Hz, 1H), 2.51- 2.44 (m, 1H), 2.35 (s, 3H), 2.29-2.23 (m, 1H). LC/MS (ESI) m/z: 614 (M+H)⁺. RT (Method A): 1.64 min.

The following compounds were prepared based on Scheme 100:

| Cmpd | | | |
|---|---|---|---|
| # | Reactant A | Reactant B | Reactant C |
| 322 | 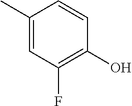 | 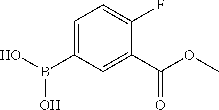 | 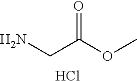 |
| 327 | 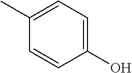 | 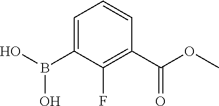 | 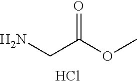 |
| 328 | 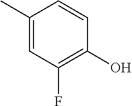 | 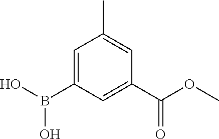 | 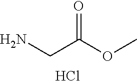 |
| 371 | 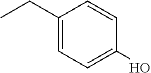 | 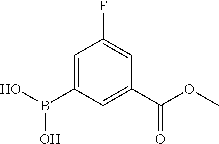 | 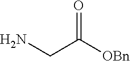 [SMILES: NCC(=O)OCc1ccccc1] |

| Cmpd | | | |
|---|---|---|---|
| # | Reactant D | Reactant E | Characterization Data |
| 322 | 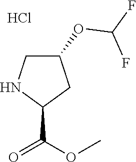 | 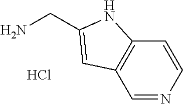 [SMILES: NCc1cc2cnccc2[nH]1 ; Cl] | ¹H NMR (400 MHz, CD₃OD): δ 8.65 (s, 1H), 8.02 (d, J = 5.9 Hz, 1H), 7.36 (dd, J = 5.9, 3.2 Hz, 1H), 7.27 (d, J = 5.9 Hz, 1H), 7.22 (d, J = 10.2 Hz, 1H), 7.15-7.11 (m, 1H), 7.06 (d, J = 11.5 Hz, 1H), 6.99 (t, J = 8.3 Hz, 2H), 6.69-6.52 (m, 1H), 6.51-6.31 (m, 1H), 5.02 (s, 1H), 4.69 (d, J = 16.0 Hz, 1H), 4.57 (d, J = 7.7 Hz, 1H), 4.51 (d, J = 15.8 Hz, 1H), 4.29 (d, J = 16.9 Hz, 1H), 4.21 (d, J = 16.8 Hz, 1H), 3.97-3.93 (m, 1H), 3.84 (d, J = 12.0 Hz, 1H), 2.51-2.44 (m, 1H), 2.33 (s, 3H), 2.29-2.22 (m, 1H). LC/MS (ESI) m/z: 614 (M + H)⁺. RT (Method A): 1.60 min. |
| 327 | 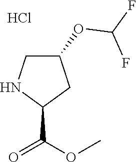 | 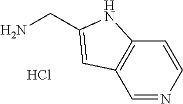 [SMILES: NCc1cc2cnccc2[nH]1 ; Cl] | H NMR (400 MHz, CD₃OD): δ 8.71-8.62 (m, 1H), 8.09-7.93 (m, 1H), 7.59- 7.51 (m, 1H), 7.37-7.26 (m, 1H), 7.23-7.15 (m, 4H), 6.90 (d, J = 8.5 Hz, 2H), 6.71-6.33 (m, 2H), 5.07-4.92 (m, 1H), 4.75-4.69 (m, 1H), 4.62-4.47 (m, 2H), 4.36-4.17 (m, 2H), 4.02-3.85 (m, 2H), 2.52-2.36 (m, 1H), 2.31 (s, 3H), 2.30-2.23 (m, 1H). LC/MS (ESI): m/z 596 (M + H)⁺. |
| 328 | 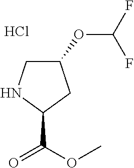 | 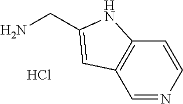 [SMILES: NCc1cc2cnccc2[nH]1 ; Cl] | ¹H NMR (400 MHz, CD₃OD): δ 8.63 (s, 1H), 7.97 (d, J = 5.8 Hz, 1H), 7.39 (s, 1H), 7.22 (s, 1H), 7.18 (d, J = 5.8 Hz, 1H), 7.05 (d, J = 11.0 Hz, 1H), 6.99 (d, J = 7.1 Hz, 2H), 6.93 (s, 1H), 6.70-6.50 (m, 1H), 6.50-6.32 (m, 1H), 5.02 (s, 1H), 4.69 (d, J − 15.9 Hz, 1H), 4.63-4.56 (m, 1H), 4.51 (d, J = 16.0 Hz, 1H), 4.23 (d, J = 16.7 Hz, 1H), 4.15 (d, J = 16.8 Hz, 1H), 3.99-3.94 (m, 1H), 3.86 (d, J = 11.8 Hz, 1H), 2.51-2.44 (m, 1H), 2.34 (s, 3H), 2.33 (s, 3H), 2.30-2.23 (m, 1H). LC/MS (ESI) m/z: 610 (M + H)⁺. RT (Method A): 1.56 min. |
| 371 | 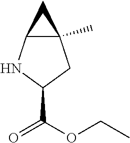 | 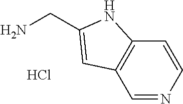 [SMILES: NCc1cc2cnccc2[nH]1 ; Cl] | ¹H NMR (400 MHz, CD₃OD): δ 8.68-8.65 (m, 1H), 8.04-8.01 (m, 1H), 7.30- 7.25 (m, 3H), 7.24-7.21 (m, 2H), 6.98-6.94 (m, 2H), 6.87-6.82 (m, 1H), 6.53-6.50 (m, 1H), 4.85-4.82 (m, 1H), 4.65-4.60 (m, 1H), 4.51-4.46 (m, 1H), 4.41-4.35 (m, 1H), 4.31-4.25 (m, 1H), 3.39-3.36 (m, 1H), 2.67-2.65 (m, 1H), 2.63-2.61 (m, 1H), 2.46-2.39 (m, 1H), 2.22-2.17 (m, 1H), 1.30 (s, 3H), 1.25- 1.21 (m, 4H), 0.84-0.80 (m, 1H). LC/MS (ESI) m/z: 570 (M + H)⁺. RT (Method A): 1.74 min. |

### Scheme 101. Synthesis of (2S,4R*)-N-((1H-pyrrolo[3,2-c]pyridin-2-yl)methyl)-1-((3-(2-fluoro-4-methylphenoxy)benzoyl)glycyl)-4-(methoxymethyl)pyrrolidine-2-carboxamide (Compound 318)

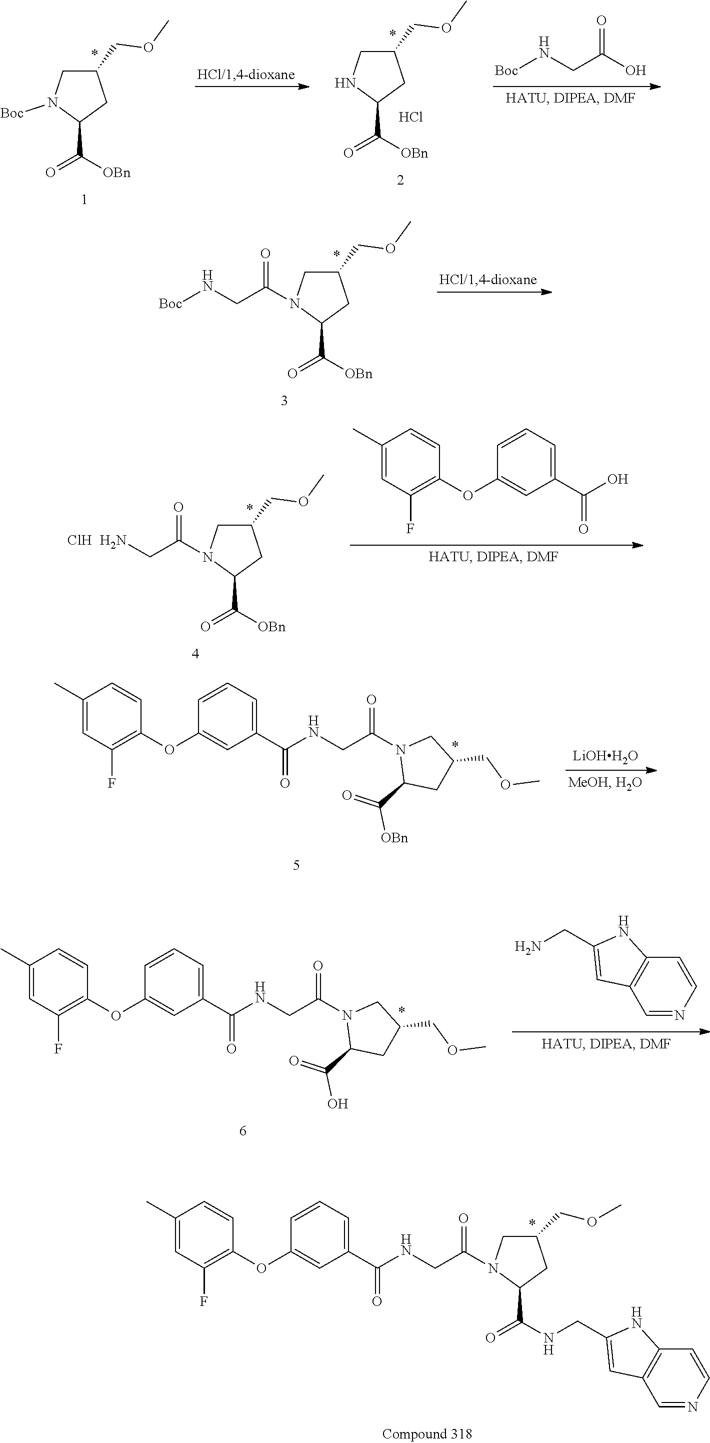

### Step 1: Benzyl (2S,4R*)-4-(methoxymethyl)pyrrolidine-2-carboxylate hydrochloride (2)

A solution of 2-benzyl 1-(tert-butyl) (2S,4R)-4-(methoxymethyl)pyrrolidine-1,2-dicarboxylate (380 mg, 1.1 mmol) in HCl/1,4-dioxane (3 mL, 4M) was stirred under N₂ atmosphere at room temperature for 2 hours. The reaction mixture was concentrated under reduced pressure to dryness to give the title compound (310 mg, yield 98.6%) as a yellow solid, which was used directly in the next step without further purification. LC/MS (ESI) m/z: 250 (M+H)⁺.

### Step 2: Benzyl (2S,4R*)-1-((tert-butoxycarbonyl)glycyl)-4-(methoxymethyl) pyrrolidine-2-carboxylate (3)

To a solution of benzyl (2S,4R)-4-(methoxymethyl)pyrrolidine-2-carboxylate hydrochloride (310 mg, 1.1 mmol) in DMF (150 mL) was added (tert-butoxycarbonyl)glycine (200 mg, 1.1 mmol) under N₂ atmosphere and the reaction mixture was stirred at room temperature for 10 minutes. The mixture was diluted with EtOAc, washed with water and brine, dried over anhydrous Na₂SO₄, filtered, and concentrated under reduced pressure to dryness. The residue was purified by flash chromatography (silica gel, 0-40% EtOAc in PE) to give the title compound (200 mg, yield 44.8%) as a white solid. LC/MS (ESI) m/z: 407 (M+H)⁺.

### Step 3: Benzyl (2S,4R)-1-glycyl-4-(methoxymethyl)pyrrolidine-2-carboxylate hydrochloride (4)

A solution of benzyl (2S,4R*)-1-((tert-butoxycarbonyl)glycyl)-4-(methoxymethyl) pyrrolidine-2-carboxylate (100 mg, 0.25 mmol) in HCl/1,4-dioxane (2 mL, 4M) was stirred under N₂ atmosphere at room temperature for 2 hours. The reaction mixture was concentrated under reduced pressure to dryness to give the title compound (82 mg, yield 95.7%) as a yellow syrup, which was used directly in the next step without further purification. LC/MS (ESI) m/z: 307 (M+H)⁺.

### Step 4: Benzyl (2S,4R*)-1-((3-(2-fluoro-4-methylphenoxy)benzoyl)glycyl)-4-(methoxymethyl)pyrrolidine-2-carboxylate (5)

To a solution of benzyl (2S,4R*)-1-glycyl-4-(methoxymethyl)pyrrolidine-2-carboxylate hydrochloride (82 mg, 0.24 mmol) in DMF (1 mL) was added 3-(2-fluoro-4-methylphenoxy)benzoic acid (59 mg, 0.24 mmol), HATU (106 mg, 0.28 mmol) and DIPEA (155 mg, 1.2 mmol) and the mixture was stirred at room temperature for 10 minutes. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (50 mg, yield 39.0%) as a white solid.

### Step 5: (2S,4R*)-1-((3-(2-fluoro-4-methylphenoxy)benzoyl)glycyl)-4-(methoxymethyl) pyrrolidine-2-carboxylic acid (6)

To a solution of benzyl (2S,4R*)-1-((3-(2-fluoro-4-methylphenoxy)benzoyl)glycyl)-4-(methoxymethyl)pyrrolidine-2-carboxylate (50 mg, 0.094 mmol) in MeOH/water (1 mL, v/v=1/1) was added LiOH H₂O (24 mg, 0.60 mmol) and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (40 mg, yield 96.2%) as a white solid, which was used directly in the next step without further purification. LC/MS (ESI) m/z: 445 (M+H)⁺.

### Step 6: (2S,4R*)-N-((1H-pyrrolo[3,2-c]pyridin-2-yl)methyl)-1-((3-(2-fluoro-4-methylphenoxy)benzoyl)glycyl)-4-(methoxymethyl)pyrrolidine-2-carboxamide (Compound 318)

To a mixture of (2S,4R*)-1-((3-(2-fluoro-4-methylphenoxy)benzoyl)glycyl)-4-(methoxymethyl)pyrrolidine-2-carboxylic acid (40 mg, 0.090 mmol) and (LH-pyrrolo[3,2-c]pyridin-2-yl)methanamine (13 mg, 0.090 mmol) in DMF (1.5 mL) was added HATU (41 mg, 0.11 mmol) and DIPEA (58 mg, 0.45 mmol) under N₂ atmosphere and the reaction mixture was stirred at room temperature for 10 minutes. The residue was purified by flash chromatography (silica gel, 0-10% MeOH in DCM) and further purified by prep-HPLC to give Compound 318 (15 mg, yield 29.1%) as a white solid. ¹H NMR (400 MHz, CD₃OD): δ 8.77 (d, J=19.3 Hz, 1H). 8.08 (t, J=9.5 Hz, 1H), 7.53 (d, J=7.7 Hz, 1H), 7.44-7.37 (m, 3H), 7.10-6.99 (m, 4H), 6.66 (d, J=13.4 Hz, 1H₁), 4.65-4.60 (m, 2H), 4.56 (dd, J=8.4, 3.6 Hz, 1H), 4.26-4.10 (m, 2H), 3.96-3.90 (m, 1H), 3.52-3.39 (m, 3H), 3.37 (d, J=7.2 Hz, 3H), 2.82-2.69 (m, 1H), 2.34 (s, 3H), 2.16-2.11 (m, 1H), 2.10-2.03 (m, 1H). LC/MS (ESI) m/z: 574 (M+H)⁺. RT (Method A): 1.43 min.

Compounds 367 was prepared based on Scheme 101:

| Cmpd # | Reactant A | Reactant B | Reactant C |
|---|---|---|---|
| 367 | 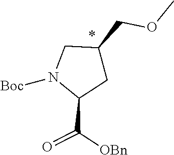 [SMILES: COC[C@H]1C[C@@H](C(=O)OCc2ccccc2)N(C(=O)OC(C)(C)C)C1] | 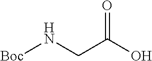 [SMILES: CC(C)(C)OC(=O)NCC(=O)O] | 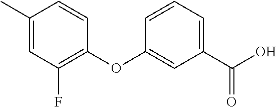 [SMILES: Cc1ccc(Oc2cccc(C(=O)O)c2)c(F)c1] |

| Cmpd # | Reactant D | Characterization |
|---|---|---|
| 367 | 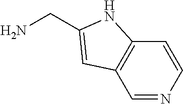 [SMILES: NCc1cc2cnccc2[nH]1] | ¹H NMR (400 MHz, CD₃OD): δ 8.66 (s, 1H), 7.97 (d, J = 5.8.3 Hz, 1H), 7.55 (d, J = 7.7 Hz, 1H), 7.41 (dd, J = 9.9, 5.5 Hz, 2H), 7.18 (d, J = 5.8 Hz, 1H), 7.12-7.04 (m, 2H), 7.03-6.96 (m, 2H), 6.52 (d, J = 24.6 Hz, 1H), 4.64 (m, 2H), 4.50 (d, J = 15.9 Hz, 1H), 4.43 (t, J = 8.3 Hz, 1H), 4.26 (d, J = 16.7 Hz, 1H), 4.13 (d, J = 16.6 Hz, 1H), 3.94-3.88 (m, 1H), 3.43 (dt, J = 16.3, 9.4 Hz, 3H), 3.32 (s, 1H), 3.30-3.30 (m, 1H), 2.68-2.59 (m, 1H), 2.45-2.37 (m, 1H), 2.34 (d, J = 6.1 Hz, 3H), 1.79 (dt, J = 12.5, 9.3 Hz, 1H). LC/MS (ESI) m/z: 574 (M + H)⁺. RT (Method A): 1.41 min. |

### Scheme 102. Synthesis of (2S,4R)-N-((1H-pyrrolo[3,2-c]pyridin-2-yl)methyl)-4-(difluoromethoxy)-1-((3-((2-fluoro-4-methylphenyl)amino)benzoyl)glycyl) pyrrolidine-2-carboxamide (Compound 324)

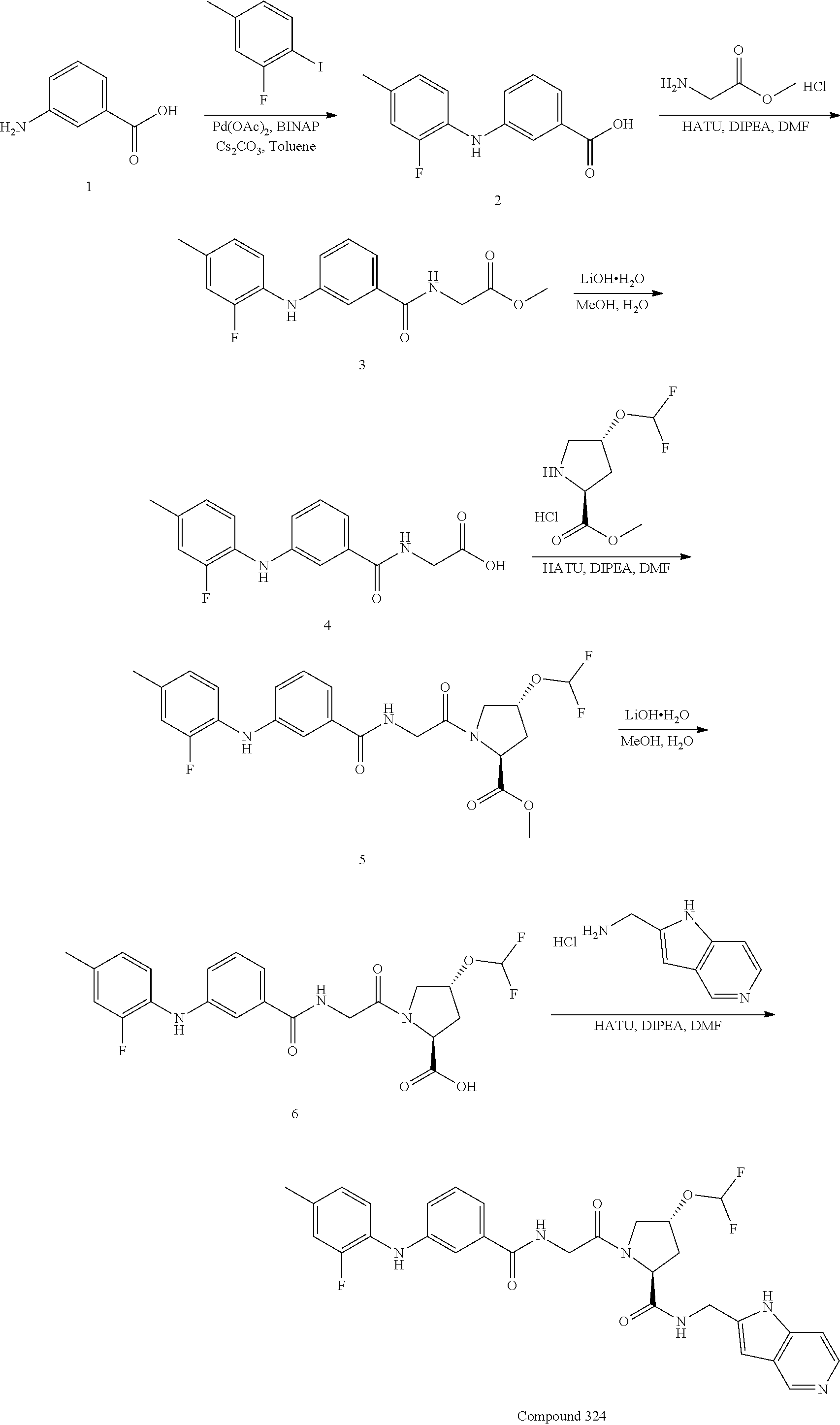

### Step 1: 3-((2-Fluoro-4-methylphenyl)amino)benzoic acid (2)

To a mixture of 3-aminobenzoic acid (1.37 g, 10.0 mmol) and 2-fluoro-1-iodo-4-methylbenzene (2.36 g, 10.0 mmol) in toluene (15 mL) was added Cs₂CO₃ (1.7 g, 15.0 mmol), BINAP (1.25 g, 2.0 mmol) and Pd(OAc)₂ (225 mug, 1.0 mmol) under N₂ atmosphere, the reaction mixture was degassed under N₂ atmosphere for three times and stirred at 100° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-100% EtOAc in PE) to give the title compound (100 mg, yield 4.1%) as a yellow solid. LC/MS (ESI) m/z: 246 (M+H)⁺.

### Step 2: Methyl (3-((2-fluoro-4-methylphenyl)amino)benzoyl)glycinate (3)

To a mixture of 3-((2-fluoro-4-methylphenyl)amino)benzoic acid (95 mg, 0.39 mmol) and methyl glycinate hydrochloride (58 mg, 0.46 mmol) in DMF (1 mL) was added DIPEA (155 mg, 1.2 mmol) and HATU (175 mg, 0.46 mmol) under N₂ atmosphere and the mixture was stirred at room temperature for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-60% EtOAc in PE) to give the title compound (90 mg, yield 72.9%) as a yellow solid. LC/MS (ESI) m/z: 317 (M+H)⁺.

### Step 3: (3-((2-Fluoro-4-methylphenyl)amino)benzoyl)glycine (4)

To a solution of methyl (3-((2-fluoro-4-methylphenyl)amino)benzoyl)glycinate (86 mg, 0.27 mmol) in MeOH (1 mL) and water (0.5 mL) was added LiOH·H₂O (33 mg, 0.80 mmol) at 0° C. and the mixture was stirred at room temperature for 4 hours. The mixture was acidified with 1N aq·HCl to pH˜4 and extracted with EtOAc twice. The combined organic layers were washed with brine, dried over anhydrous Na₂SO₄, filtered, and concentrated to give the title compound (80 mg, yield 97.6%) as a white solid. LC/MS (ESI) (m/z): 303 (M+H)⁺.

### Step 4: Methyl (2S,4R)-4-(difluoromethoxy)-1-((3-((2-fluoro-4-methylphenyl)amino) benzoyl)glycyl)pyrrolidine-2-carboxylate (5)

To a mixture of (3-((2-fluoro-4-methylphenyl)amino)benzoyl)glycine (80 mg, 0.26 mmol) and methyl (2S,4R)-4-(difluoromethoxy)pyrrolidine-2-carboxylate hydrochloride (90 mg, 0.39 mmol) in DMF (1 mL) was added DIPEA (104 mg, 0.80 mmol) and HATU (133 mg, 0.35 mmol) under N₂ atmosphere and the mixture was stirred at room temperature for 1 hour. The mixture was diluted with EtOAc, washed with water and brine, dried over anhydrous Na₂SO₄, filtered, and concentrated under reduced pressure to dryness. The residue was purified by flash chromatography (silica gel, 0-90% EtOAc in PE) to give the title compound (95 mg, yield 76.2%) as a yellow solid. LC/MS (ESI) m/z: 480 (M+H)⁺.

### Step 5: (2S,4R)-4-(difluoromethoxy)-1-((3-((2-fluoro-4-methylphenyl)amino)benzoyl) glycyl)pyrrolidine-2-carboxylic acid (6)

To a solution of methyl (2S,4R)-4-(difluoromethoxy)-1-((3-((2-fluoro-4-methylphenyl) amino)benzoyl)glycyl)pyrrolidine-2-carboxylate (90 mg, 0.19 mmol) in MeOH (1 mL) and water (0.5 mL) was added LiOH·H₂O (33 mg, 0.80 mmol) at 0° C. and the mixture was stirred at room temperature for 4 hours. The mixture was acidified with 1N aq·HCl to pH˜4 and extracted with EtOAc twice. The combined organic layers were washed with brine, dried over anhydrous Na₂SO₄, filtered, and concentrated to give the title compound (55 mg, yield 62.2%) as a white solid. LC/MS (ESI) (m/z): 466 (M+H)⁺.

### Step 6: (2S,4R)-N-((1H-pyrrolo[3, 2-c]pyridin-2-yl)methyl)-4-(difluoromethoxy)-1-((3-((2-fluoro-4-methylphenyl)amino)benzoyl)glycyl)pyrrolidine-2-carboxamide (Compound 324)

To a mixture of (2S,4R)-4-(difluoromethoxy)-1-((3-((2-fluoro-4-methylphenyl)amino) benzoyl)glycyl)pyrrolidine-2-carboxylic acid (42 mg, 0.09 mmol) and (1H-pyrrolo[3,2-c]pyridin-2-yl)methanamine hydrochloride (33 mg, 0.18 mmol) in DMF (0.5 mL) was added DIPEA (39 mg, 0.30 mmol) and HATU (38 mg, 0.1 mmol) under N₂ atmosphere and the reaction mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=8:1) and further purified by prep-HPLC to give Compound 324 (10 mg, yield 18.7%) as a white solid. ¹H NMR (400 MHz, CD₃OD): δ 8.67 (d, J=24.9 Hz, 1H), 8.01 (m, 1H), 7.44 (s, 1H), 7.29 (d, J=5.1 Hz, 2H), 7.19 (m, 2H), 7.12 (m, 1H), 6.95 (d, J=12.0 Hz, 1H), 6.88 (d, J=8.4 Hz, 1H), 6.52 (m, 2H), 5.04 (s, 1H), 4.71 (d, J=16.0 Hz, 1H), 4.62-4.49 (m, 2H), 4.29-4.10 (m, 2H), 3.99 (m, 1H), 3.88 (d, J=11.3 Hz, 1H), 2.54-2.41 (m, 1H), 2.32-2.24 (m, 1H), 2.29 (s, 3H). LC/MS (ESI) m/z: 595 (M+H)⁺. RT (Method A): 1.40 min.

### Scheme 103. Synthesis of (2S,4R)-4-(difluoromethoxy)-N-((5-methyl-4,5,6,7-tetrahydrothieno[3,2-c]pyridin-2-yl)methyl)-1-((phenoxathiine-3-carbonyl)glycyl) pyrrolidine-2-carboxamide (Compound 347)

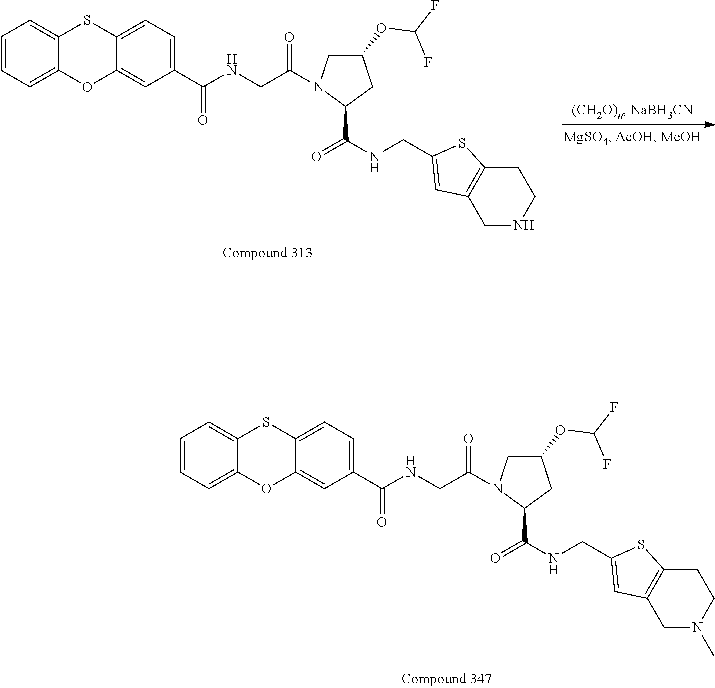 [SMILES: O=C(NCC(=O)N1C[C@H](OC(F)F)C[C@H]1C(=O)NCc1cc2c(s1)CCNC2)c1ccc2c(c1)Oc1ccccc1S2 ; CN1CCc2sc(CNC(=O)[C@@H]3C[C@@H](OC(F)F)CN3C(=O)CNC(=O)c3ccc4c(c3)Oc3ccccc3S4)cc2C1]

To solution of (2S,4R)-4-(difluoromethoxy)-1-((phenoxathiine-3-carbonyl)glycyl)-N-((4,5,6,7-tetrahydrothieno[3,2-c]pyridin-2-yl)methyl)pyrrolidine-2-carboxamide (Compound 313; 11 mg, 0.018 mmol) in MeOH (2 mL) was added paraformaldehyde ((CH₂O)ₙ, 43 mg), MgSO₄ (87 mg, 0.72 mmol), AcOH (15 mg) and NaBH₃CN (43 mg, 0.68 mmol) and the reaction mixture was stirred under N₂ atmosphere at room temperature overnight. The reaction mixture was quenched with saturated aq·NH₄CL solution and extracted with DCM twice. The combined organic layers were dried over Na₂SO₄, filtered, and concentrated under reduced pressure to dryness. The residue was purified by prep-HPLC to give Compound 347 (4.0 mg, yield 39.0%) as a white solid. ¹H NMR (400 MHz, CD₃OD): δ 7.55-7.50 (m, 1H), 7.49-7.44 (m, 1H), 7.21-7.11 (m, 3H), 7.08-7.00 (m, 2H), 6.69-6.27 (m, 2H), 5.03-4.96 (m, 1H), 4.55 (t, J=7.7 Hz, 1H), 4.50-4.38 (m, 2H), 4.25-4.09 (m, 2H), 3.99-3.89 (m, 1H), 3.85-3.75 (m, 1H), 3.37 (dd, J=25.1, 10.4 Hz, 2H), 2.84-2.75 (m, 2H), 2.72-2.64 (m, 2H), 2.47-2.35 (m, 1H), 2.39 (s, 3H), 2.27-2.16 (m, 1H). LC/MS (ESI) m/z: 629 (M+H)⁺. RT (Method A): 1.53 min.

### Scheme 104. Synthesis of (2S,4R)-N-((1H-pyrrolo[3,2-c]pyridin-2-yl)methyl)-4-(difluoromethoxy)-1-((8-methyldibenzo[b,d]furan-2-carbonyl)glycyl)pyrrolidine-2-carboxamide (Compound 334)

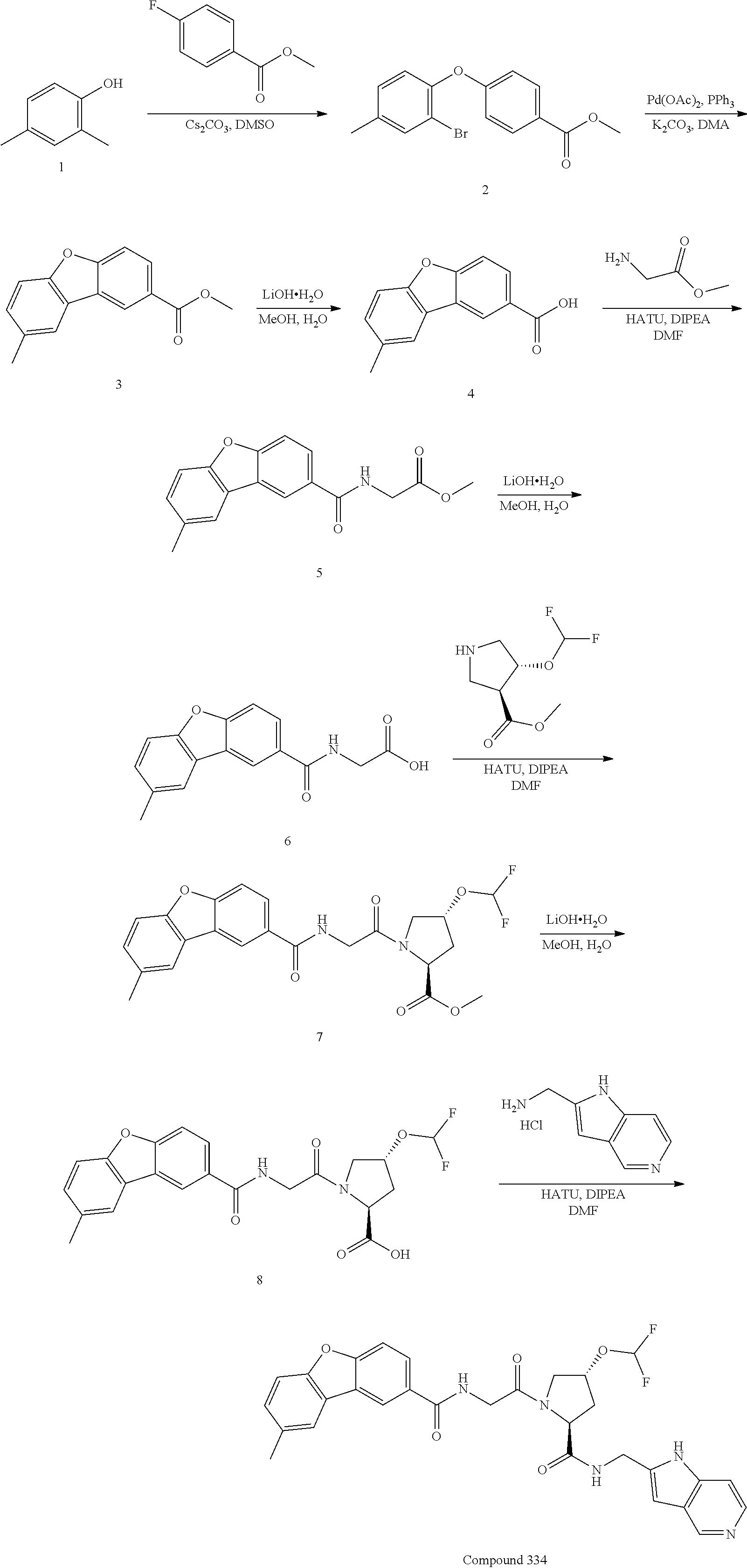

### Step 1: Methyl 4-(2-bromo-4-methylphenoxy)benzoate (2)

To a mixture of 2-bromo-4-methylphenol (1.0 g, 5.37 mmol) and methyl 4-fluorobenzoate (824 mg, 5.34 mmol) in DMSO (20 mL) was added Cs₂CO₃ (5.22 g, 16.02 mmol) at 25° C. under N₂ atmosphere, the reaction mixture was degassed under N₂ atmosphere for three times and stirred under N₂ atmosphere at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-30% EtOAc in PE) to give the title compound (410 mg, yield 24.0%) as a colorless oil. LC/MS (ESI) m/z: 321 (M+H)⁺.

### Step 2: Methyl 8-methyldibenzo-2-carboxylate (3)

To a solution of methyl 4-(2-bromo-4-methylphenoxy)benzoate (400 mg, 1.25 mmol) in DMA (15 mL) was added Pd(OAc)₂ (11 mg, 0.05 mmol), PPh₃ (27 mg, 0.10 mmol) and K₂CO₃ (224 mg, 1.62 mmol) at 25° C. under N₂ atmosphere, the reaction mixture was degassed under N₂ atmosphere for three times and stirred under N₂ atmosphere at 130° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-7% EtOAc in PE) to give the title compound (120 mg, yield 40.1%) as a colorless oil. ¹H NMR (400 MHz, CDCl₃) δ 8.61 (d, J=1.6 Hz, 1H), 8.12 (dd, J=8.6, 1.7 Hz, 1H), 7.75 (s, 1H), 7.52 (d, J=8.6 Hz, 1H), 7.43 (d, J=8.4 Hz, 1H), 7.28-7.25 (m, 1H), 3.94 (s, 3H), 2.49 (s, 3H).

### Step 3: Methyl 8-methyldibenzo-2-carboxylate (4)

To a solution of methyl 8-methyldibenzo-2-carboxylate (120 mg, 0.50 mmol) in MeOH (2 mL) and water (1 mL) was added LiOH (48 mg, 2.00 mmol) and the mixture was stirred at room temperature for 1 hour. The mixture was acidified with 1N aq·HCl to pH˜-3 and extracted with EtOAc twice. The combined organic layers were washed with brine, dried over anhydrous Na₂SO₄, filtered, and concentrated under reduced pressure to dryness to give the title compound (110 mg, yield 97.3%) as a white solid, which was used directly in the next step without further purification. LC/MS (ESI) m/z: 226 (M+H)⁺.

### Step 4: Methyl (8-methyldibenzo-2-carbonyl)glycinate (5)

To a mixture of methyl 8-methyldibenzo-2-carboxylate (110 mg, 0.48 mol) and methyl glycinate (71 mg, 0.79 mmol) in DMF (5 mL) was added DIPEA (228 mg, 1.76 mmol) and HATU (218 mg, 8.46 mol) and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-23% EtOAc in PE) to give the title compound (100 mg, yield 69.1%) as a colorless oil. LC/MS (ESI) m/z: 298 (M+H)⁺.

### Step 5: (8-Methyldibenzo-2-carbonyl)glycine (6)

To a solution of methyl (8-methyldibenzo-2-carbonyl)glycinate (100 mg, 0.33 mol) in McOH (2 mL) and water (1 mL) was added LiOH (32 mg, 1.33 mol), and the mixture was stirred at room temperature for 1 hour. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (72 mg, yield 75.5%) as a white solid, which was used directly in the next step without further purification. LC/MS (ESI) m/z: 284 (M+H)⁺.

### Step 6: Methyl (2S,4R)-4-(difluoromethoxy)-1-((8-methyldibenzo[b,d]furan-2-carbonyl)glycyl)pyrrolidine-2-carboxylate (7)

To a mixture of (8-methyldibenzo-2-carbonyl)glycine (35 mg, 0.12 mol) and methyl (3S,4R)-4-(difluoromethoxy)pyrrolidine-3-carboxylate (25 mg, 0.10 mmol) in DMF (2 mL) was added DIPEA (52 mg, 0.41 mmol) and HATU (52 mg, 0.13 mol). The mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-49% EtOAc in PE) to give the title compound (55 mg, yield 96.7%) as a yellow oil. LC/MS (ESI) m/z: 461 (M+H)⁺.

### Step 7: (2S,4R)-4-(difluoromethoxy)-1-((8-methyldibenzo[b,d]furan-2-carbonyl) glycyl)pyrrolidine-2-carboxylic acid (8)

To a solution of methyl (2S,4R)-4-(difluoromethoxy)-1-((8-methyldibenzo[b,d]furan-2-carbonyl)glycyl)pyrrolidine-2-carboxylate (55 mg, 0.12 mol) in MeOH (2 mL) and water (1 mL) was added LiOH (10 mg, 0.41 mol), and the mixture was stirred at room temperature for 1 hour. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (50 mg, yield 93.7%) as a yellow oil, which was used directly in the next step without further purification. LC/MS (ESI) m/z: 447 (M+H)⁺.

### Step 8: (2S,4R)-N-((JH-pyrrolo[3,2-c]pyridin-2-yl)methyl)-4-(difluoromethoxy)-1-((8-methyldibenzo[b,d]furan-2-carbonyl)glycyl)pyrrolidine-2-carboxamide (Compound 334)

To a mixture of (2S,4R)-4-(difluoromethoxy)-1-((8-methyldibenzo[b,d]furan-2-carbonyl)glycyl)pyrrolidine-2-carboxylic acid (50 mg, 0.10 mol) and (1H-pyrrolo[3,2-c]pyridin-2-yl)methanamine (27 mg, 0.14 mmol) in DMF (3 mL) was added DIPEA (55.3 mg, 0.14 mmol) and HATU (58 mg, 0.44 mol) and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=8:1) and further purified by prep-HPLC to give Compound 334 (5 mug, yield 7.7%) as a white solid. ¹H NMR (400 MHz, CD₃OD): δ 8.56-8.49 (m, 2H), 7.99-7.94 (m, 1H), 7.81 (d, J=5.8 Hz, 2H), 7.59 (d, J=8.6 Hz, 1H), 7.49 (d, J=8.4 Hz, 1H), 7.35 (dd, J=8.4, 1.2 Hz, 1H), 7.14 (d, J=5.8 Hz, 1H), 6.62-6.35 (m, 2H), 5.11-5.01 (m, 1H), 4.71-4.53 (m, 3H), 4.34-4.19 (m, 2H), 4.05-4.00 (m, 1H), 3.95-3.89 (m, 1H), 2.52-2.48 (m, 1H), 2.50 (s, 3H), 2.33-2.25 (m, 1H). LC/MS (ESI) m/z: 576 (M+H)⁺. RT (Method A): 1.43 min.

### Scheme 105. Synthesis of (2S,4R)-N-((1H-pyrrolo[3,2-c]pyridin-2-yl)methyl)-4-(difluoromethoxy)-1-((9-fluorodibenzo[b,d]furan-2-carbonyl)glycyl)pyrrolidine-2-carboxamide (Compound 369)

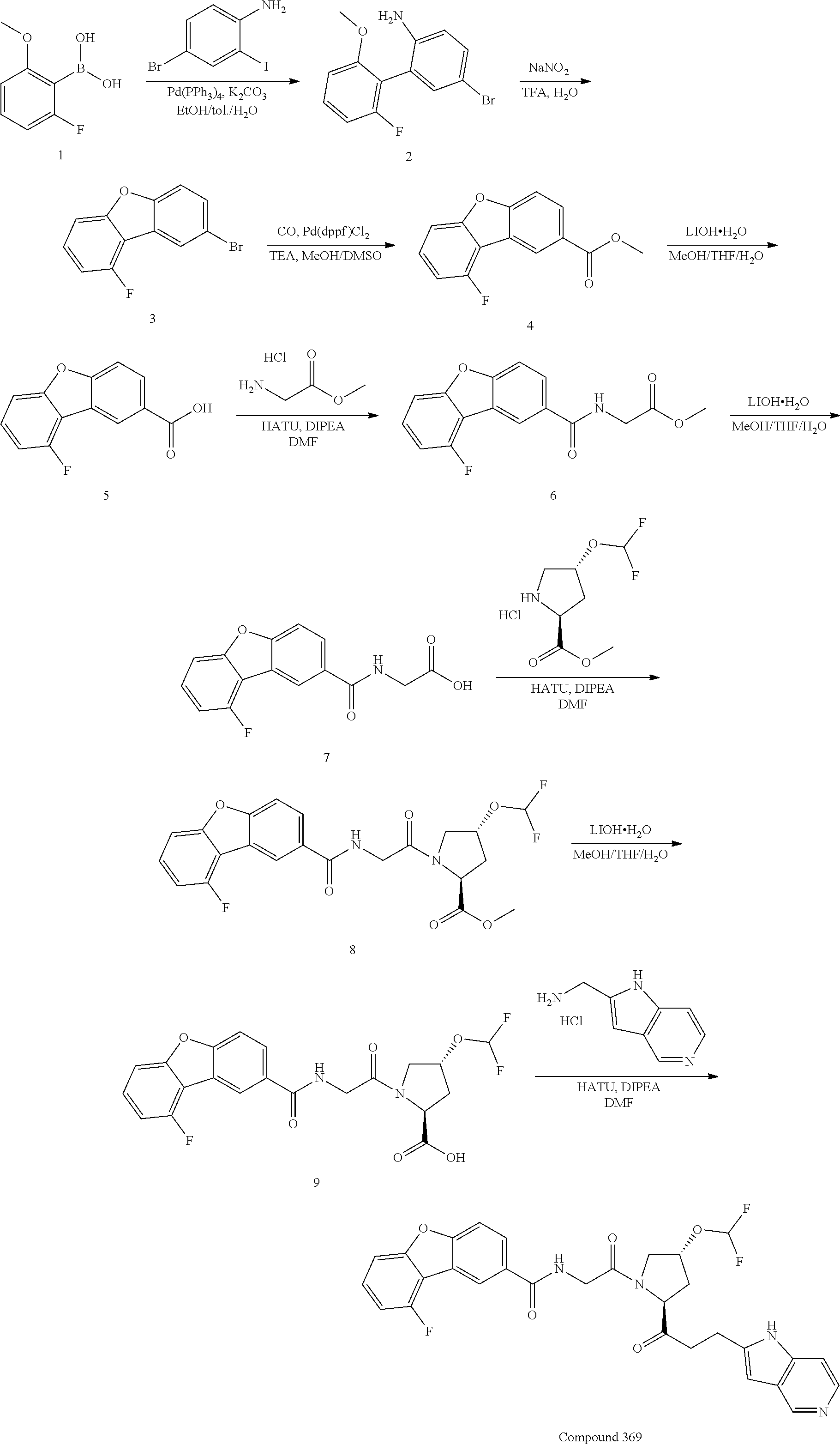

### Step 1: 5-Bromo-2′-fluoro-6′-methoxy-[1,1′-biphenyl]-2-amine (2)

To a mixture of (2-fluoro-6-methoxyphenyl)boronic acid (740 mg, 4.35 mmol) and 4-bromo-2-iodoaniline (1 g, 3.35 mmol) in EtOH/toluene/water (11 mL, v/v/v=5/5/1) was added K₂C₀₃ (1.4 g, 10.1 mmol) and Pd(PPh₃)₄ (387 mg, 0.43 mmol) at 0° C. under N₂ atmosphere, the reaction mixture was degassed under N₂ atmosphere for three times and stirred at 80° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (786 mg, yield 79.3%) as a yellow solid. LC/MS (ESI) m/z: 296 (M+H)⁺.

### Step 2: 8-Bromo-1-fluorodibenzo[b,d]furan (3)

To a solution of 5-bromo-2′-fluoro-6′-methoxy-[1,1′-biphenyl]-2-amine (350 mg, 1.17 mmol) in TFA (4 mL) was added aq·NaNO₂ solution (0.4 mL, 1.4 mmol, 3.5 M) drop-wisely at 0° C. and the reaction solution was stirred at 70° C. for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (147 mg, yield 47.6%) as a white solid. ¹H NMR (400 MHz, CDCl₃) δ 8.19 (d, J=2.0 Hz, 1H), 7.58 (d, J=6.7 Hz, 1H), 7.44 (d, J=2.6 Hz, 1H), 7.43-7.40 (m, 1H), 7.37 (d, J=8.2 Hz, 1H), 7.06 (s, 1H).

### Step 3: Methyl 9-fluorodibenzo[b,d]furan-2-carboxylate (4)

To a mixture of 8-bromo-1-fluorodibenzo[b,d]furan (127 mg, 0.48 mmol) in MeOH (2 mL) and DMSO (2 mL) was added TEA (146 mg, 1.44 mmol) and Pd(dppf)C₁₂ (35 mg, 0.05 mmol). The reaction mixture was degassed under N₂ atmosphere for three times and stirred under CO atmosphere at 85° C. overnight. The mixture was filtered, and the filtrate was concentrated under reduced pressure to dryness. The residue was purified by flash chromatography on (silica gel, 0-40% EtOAc in PE) to give the title compound (117 mg, yield 99.3%) as a yellow solid. LC/MS (ESI) m/z: 245 (M+H)⁺.

### Step 4: 9-Fluorodibenzo[b,d]furan-2-carboxylic acid (S)

To a solution of methyl 9-fluorodibenzo[b,d]furan-2-carboxylate (117 mg, 0.48 mmol) in MeOH/THF/water (4 mL, v/v/v=2/1/1) was added LiOH·H₂O (60 mg, 1.44 mmol) under N₂ atmosphere and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to dryness to give the title compound (110 mg, yield 99.8%) as a yellow solid, which was used directly in the next step without further purification. LC/MS (ESI) m/z: 231 (M+H)⁺.

### Step 5: Methyl (9-fluorodibenzo[b,d]furan-2-carbonyl)glycinate (6)

To a mixture of 9-fluorodibenzo[b,d]furan-2-carboxylic acid (132 mg, 0.57 mmol) and methyl glycinate hydrochloride (86 mg, 0.68 mmol) in DMF (2 mL) was added DIPEA (404 mg, 3.1 mmol) and HATU (285 mg, 0.75 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-70% EtOAc in PE) to give the title compound (132 mg, yield 76.4%) as a white solid. LC/MS (ESI) m/z: 302 (M+H)⁺.

### Step 6: (9-Fluorodibenzo[b,d]furan-2-carbonyl)glycine (7)

To a solution of methyl (9-fluorodibenzo[b,d]furan-2-carbonyl)glycinate (132 mg, 0.43 mmol) in MeOH/THF/water (3 mL, v/v/v=2/1/1) was added LiOH·H₂O (55 mg, 1.3 mmol) under N₂ atmosphere and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (125 mg, yield 99.4%) as a yellow solid, which was used directly in the next step without further purification. LC/MS (ESI) m/z: 288 (M+H)⁺.

### Step 7: Methyl (2S,4R)-4-(difluoromethoxy)-1-((9-fluorodibenzo[b,d]furan-2-carbonyl)glycyl)pyrrolidine-2-carboxylate (8)

To a mixture of (9-fluorodibenzo[b,d]furan-2-carbonyl)glycine (125 mg, 0.43 mmol) and methyl (2S,4R)-4-(difluoromethoxy)pyrrolidine-2-carboxylate hydrochloride (151 mg, 0.65 mmol) in DMF (3 mL) was added DIPEA (282 mg, 2.19 mmol) and HA T U (199 mg, 0.52 mmol) under N₂ atmosphere and the reaction mixture was stirred at room temperature for 2 hours. The mixture was diluted with EtOAc, washed with saturated aq·NaHCO₃ solution and brine, dried over anhydrous Na₂SO₄, filtered, and concentrated under reduced pressure to dryness. The residue was purified by flash chromatography (silica gel, 0-60% EtOAc in PE) to give the title compound (202 mg, yield 99.4%) as a white solid. LC/MS (ESI) m/z: 465 (M+H)⁺.

### Step 8: (2S,4R)-4-(difluoromethoxy)-1-((9-fluorodibenzo[b,d]furan-2-carbonyl)glycyl) pyrrolidine-2-carboxylic acid (9)

To a solution of methyl (2S,4R)-4-(difluoromethoxy)-1-((9-fluorodibenzo[b,d]furan-2-carbonyl)glycyl)pyrrolidine-2-carboxylate (202 mg, 0.43 mmol) in MeOH/THF/water (3 mL, v/v/v=2/1/1) was added LiOH·H₂O (55 mg, 1.28 mmol) under N₂ atmosphere and the reaction mixture was stirred at room temperature for 2 hours. The mixture was acidified with 1N aq·HCl to pH˜-3 and extracted with EtOAc twice. The combined organic layers were dried over anhydrous Na₂SO₄, filtered, and concentrated under reduced pressure to give the title compound (195 mg, yield 99.9%) as a white solid, which was used directly in the next step without further purification. LC/MS (ESI) m/z: 451 (M+H)⁺.

### Step 9: (2S,4R)-N-((1H-pyrrolo[3, 2-c]pyridin-2-yl)methyl)-4-(difluoromethoxy)-1-((9-fluorodibenzo[b,d]furan-2-carbonyl)glycyl)pyrrolidine-2-carboxamide (Compound 374)

To a mixture of (2S,4R)-4-(difluoromethoxy)-1-((9-fluorodibenzo[b,d]furan-2-carbonyl)glycyl)pyrrolidine-2-carboxylic acid (60 mg, 0.13 mmol) and (1H-pyrrolo[3,2-c]pyridin-2-yl)methanamine hydrochloride (42 mg, 0.23 mmol) in DMF (2 mL) was added DIPEA (84 mg, 0.65 mmol) and HATI (59 mg, 0.15 mmol) under N₂ atmosphere and the reaction mixture was stirred at room temperature for 1 hour. The mixture was diluted with EtOAc, washed with saturated aq·NaHCO₃ solution and brine, dried over anhydrous Na₂SO₄, filtered, and concentrated under reduced pressure to dryness. The residue was purified by prep-TLC (DCM:MeOH=7:1) and further purified by prep-HPLC to give Compound 369 (6.4 mg, yield 8.29%) as a white solid. ¹H NMR (400 MHz, CD₃OD): δ 8.59-8.53 (m, 2H), 8.05 (dd, J=8.7, 1.8 Hz, 1H), 7.83 (d, J=5.8 Hz, 1H), 7.67 (d, J=8.7 Hz, 1H), 7.55 (dd, J=8.1, 5.5 Hz, 1H), 7.49 (d, J=8.1 Hz, 1H), 7.17 (dd, J=10.7, 7.4 Hz, 2H), 6.52 (d, J=16.6 Hz, 2H), 5.07 (dd, J=7.7, 5.2 Hz, 1H), 4.71-4.55 (m, 3H), 4.29 (q, J=16.6 Hz, 2H), 4.04 (dd, J=11.4, 4.6 Hz, 1H), 3.93 (d, J=11.4 Hz, 1H), 2.56-2.47 (m, 1H), 2.35-2.25 (m, 1H). LC/MS (ESI) m/z: 580 (M+H)⁺. RT (Method A): 1.28 min.

### Scheme 106. Synthesis of (8S)-N-((1H-pyrrolo[3,2-c]pyridin-2-yl)methyl)-7-((5-(1-(3-fluorophenyl) ethyl)thiophene-2-carbonyl)glycyl)-1,4-dioxa-7-azaspiro[4.4]nonane-8-carboxamide (Compound 357)

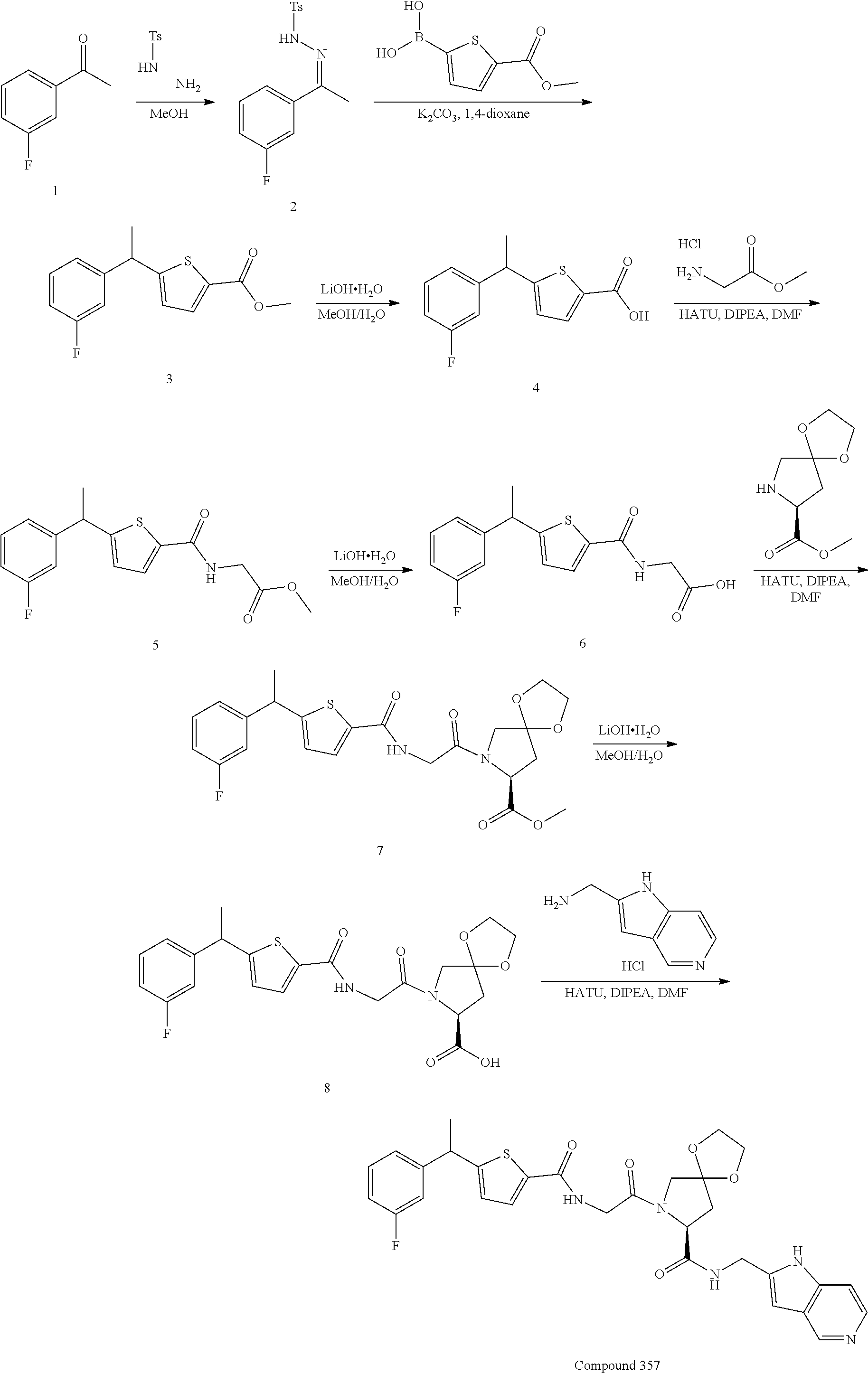

### Step 1: (Z)-N′-(1-(3-fluorophenyl)ethylidene)-4-methylbenzenesulfonohydrazide (2)

To a solution of 1-(3-fluorophenyl)ethan-1-one (1.3 g, 9.41 mmol) in MeOH (15 mL) was added 4-methylbenzenesulfonohydrazide (1.75 g, 9.39 mmol) under N₂ atmosphere and the reaction mixture was stirred at room temperature for 2 hours. The mixture was filtered, and the filter cake was washed with PE to give the title compound (2.5 g, yield 86.7%) as a white solid, which was used in the next step without purification. LC/MS (ESI) m/z: 307 (M+H)⁺.

### Step 2: Methyl 5-(1-(3-fluorophenyl)ethyl)thiophene-2-carboxylate (3)

To a mixture of (Z)-N′-(1-(3-fluorophenyl)ethylidene)-4-methylbenzenesulfonohydrazide (345 mg, 1.12 mmol) and (5-(methoxycarbonyl) thiophen-2-yl)boronic acid (250 mg, 1.34 mmol) in 1,4-dioxane (5 mL) was added K₂CO₃ (467 mg, 3.38 mmol) and the reaction mixture was degassed under N₂ atmosphere for three times and stirred at 110° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-5% EtOAc in PE) to give the title compound (100 mg, yield 33.6%) as a colorless oil. ¹H NMR (400 MHz, CDCl₃) δ 7.65-7.63 (m, 1H), 7.29 (s, 1H), 7.04 (d, J=7.6 Hz, 1H), 6.93 (t, J=9.0 Hz, 2H), 6.81 (d, J=2.6 Hz, 1H), 4.34 (q, J=7.0 Hz, 1H). 3.84 (d, J=2.0 Hz, 3H), 1.70 (dd, J=7.1, 1.9 Hz, 3H).

### Step 3: 5-(1-(3-Fluorophenyl)ethyl)thiophene-2-carboxylic acid (4)

To a solution of methyl 5-(1-(3-fluorophenyl)ethyl)thiophene-2-carboxylate (100 mg, 0.38 mmol) in MeOH/water (1.5 mL, v/v=4/1) was added LiOH 1120 (32 mg, 0.76 mmol) under N₂ atmosphere and the reaction mixture was stirred at room temperature for 2 hours. The mixture was acidified with 1N aq·HCl to pH˜3 and extracted with EtOAc twice. The combined organic layers were dried over anhydrous Na₂SO₄, filtered, and concentrated under reduced pressure to give the title compound (90 mg, yield 95.0%) as a white solid, which was used directly in the next step without further purification. LC/MS (ESI) m/z: 251 (M+H)⁺.

### Step 4: Methyl (5-(1-(3-fluorophenyl)ethyl)thiophene-2-carbonyl)glycinate (5)

To a mixture of 5-(1-(3-fluorophenyl)ethyl)thiophene-2-carboxylic acid (90 mg, 0.36 mmol) and methyl glycinate hydrochloride (54 mg, 0.43 mmol) in DMF (1 mL) was added DIPEA (232 mg, 1.79 mmol) and HATU (164 mg, 0.43 nmol) under N₂ atmosphere and the mixture was stirred at room temperature for 1 hour. The mixture was diluted with EtOAc, washed with saturated aq·NaHCO₃ solution, dried over anhydrous Na₂SO₄, filtered, and concentrated under reduced pressure to dryness. The residue was purified by flash chromatography (silica gel, 0-50% EtOAc in PE) to give the title compound (115 mg, yield 99.5%) as a colorless oil. LC/MS (ESI) m/z: 332 (M+H)⁺.

### Step 5: (5-(1-(3-Fluorophenyl)ethyl)thiophene-2-carbonyl)glycine (6)

To a solution of methyl (5-(1-(3-fluorophenyl)ethyl)thiophene-2-carbonyl)glycinate (115 mg, 0.35 mol) in MeOH/water (1.5 mL, v/v=4/1) was added LiOH·H₂O (30 mg, 0.71 mmol) under N₂ atmosphere and the reaction mixture was stirred at room temperature for 10 minutes. The mixture was acidified with 1N aq·HCl to pH˜3 and extracted with EtOAc twice. The combined organic lay ers were dried over anhydrous Na₂SO₄, filtered, and concentrated under reduced pressure to give the title compound (100 mg, yield 90.9%) as a yellow oil, which was used directly in the next step without further purification. LC/MS (ESI) n/z: 308 (M+H)⁺.

### Step 6: Methyl (8S)-7-((5-(1-(3-fluorophenyl)ethyl)thiophene-2-carbonyl)glycyl)-1,4-dioxa-7-azaspiro[4.4]nonane-8-carboxylate (7)

To a mixture of (5-(l-(3-fluorophenyl)ethyl)thiophene-2-carbonyl)glycine (90 mg, 0.29 mmol) and methyl (S)-1,4-dioxa-7-azaspiro[4.4]nonane-8-carboxylate (65 mg, 0.34 mmol) in DMF (1 mL) was added DIPEA (189 mg, 1.46 mmol) and HATU (133 mg, 0.35 mmol) under N₂ atmosphere and the mixture was stirred at room temperature for 1 hour. The mixture was diluted with EtOAc, washed with saturated aq·NaIICO₃ solution, dried over anhydrous Na₂SO₄, filtered, and concentrated under reduced pressure to dryness. The residue was purified by flash chromatography (silica gel, 0-90% EtOAc in PE) to give the title compound (55 mg, yield 39.4%) as a yellow solid. LC/MS (ESI) m/z: 477 (M+H)⁺.

### Step 7: (8S)-7-((5-(1-(3-fluorophenyl)ethyl)thiophene-2-carbonyl)glycyl)-1,4-dioxa-7-azaspiro/4.49 nonane-8-carboxylic acid (8)

To a solution of methyl (8S)-7-((5-(1-(3-fluorophenyl)ethyl)thiophene-2-carbonyl) glycyl)-1,4-dioxa-7-azaspiro[4.4]nonane-8-carboxylate (55 mg, 0.11 mol) in MeOH/water (1.5 mL, v/v=4/1) was added LiOH·H₂O (10 mg, 0.24 mmol) under N₂ atmosphere and the reaction mixture was stirred at room temperature for 1 hour. The mixture was acidified with 1N aq·HCl to pH˜3 and extracted with EtOAc twice. The combined organic layers were dried over anhydrous Na₂SO₄, filtered, and concentrated under reduced pressure to give the title compound (45 mg, yield 84.2%) as a white solid, which was used directly in the next step without further purification. LC/MS (ESI) m/z: 463 (M+H)⁺.

### Step 8: (8S)-N-((1H-pyrrolo[3,2-c]pyridin-2-yl)methyl)-7-((5-(1-(3-fluorophenyl)ethyl) thiophene-2-carbonyl)glycyl)-1,4-dioxa-7-azaspiro[4.4]nonane-8-carboxamide (Compound 357)

To a mixture of (8S)-7-((5-(1-(3-fluorophenyl)ethyl)thiophene-2-carbonyl)glycyl)-1,4-dioxa-7-azaspiro[4.4]nonane-8-carboxylic acid (45 mg, 0.097 mmol) and (1H-pyrrolo[3,2-c]pyridin-2-yl)methanamine hydrochloride (18 mg, 0.098 mmol) in DMF (1 mL) was added DIPEA (62 mg, 0.48 mmol) and HATU (44 mg, 0.11 mmol) under N₂ atmosphere and the mixture was stirred at room temperature for 1 hour. The mixture was concentered under reduced pressure to dryness. The residue was purified by prep-HPLC to give Compound 357 (4.1 mg, yield 7.1%) as a white solid. ¹H NMR (400 MHz, CD₃OD): δ 8.62 (s, 1H), 7.97 (t, J=5.7 Hz, 1H), 7.54 (dd, J=3.8, 1.2 Hz, 1H), 7.34-7.29 (m, 1H), 7.23-7.19 (m, 1H), 7.10 (d, J=7.7 Hz, 1H), 7.00 (d, J=10.2 Hz. 1H), 6.97-6.92 (m, 1H), 6.89 (d, J=3.8 Hz, 1H), 6.49 (s, 1H), 4.64 (d, J=16.1 Hz, 1H), 4.55 (dd, J=12.6, 9.0 Hz, 2H), 4.42-4.36 (m, 1H), 4.12 (d, J=3.0 Hz, 2H), 3.99 (d, J=2.6 Hz, 4H), 3.77 (s, 2H), 2.42 (dd, 1H), 2.25 (dd, 1H), 1.68 (dd, J=7.2, 2.4 Hz, 3H). LC/MS (ESI) m/z: 592 (M+H)⁺. RT (Method A): 1.36 min.

### Scheme 107. Synthesis of (2S,4R)-4-(difluoromethoxy)-N-((6-methyl-4,5,6,7-tetrahydrothieno[2,3-c]pyridin-2-yl)methyl)-1-((phenoxathiine-3-carbonyl)glycyl) pyrrolidine-2-carboxamide (Compound 346)

### Step 1: 4,5,6,7-Tetrahydrothieno[2,3-c]pyridine-2-carbonitrile hydrochloride (2)

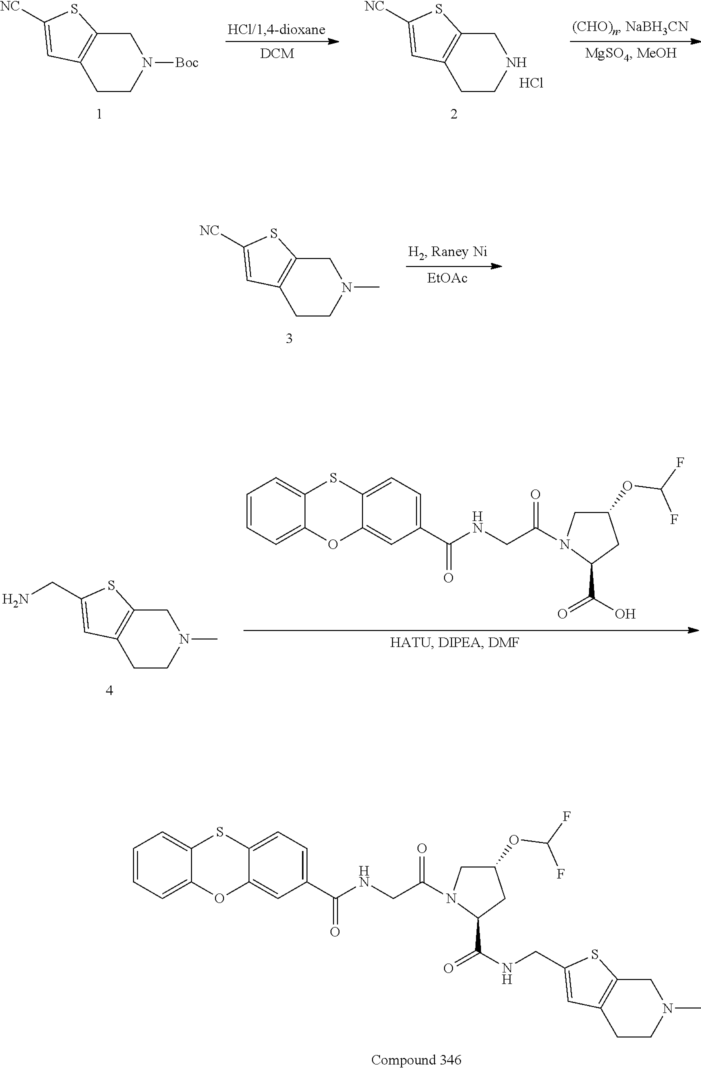

To a solution of tert-butyl 2-cyano-4,7-dihydrothieno[2,3-c]pyridine-6(5H)-carboxylate (80 mg, 0.30 mmol) in DCM (1 mL) was added HCl/1,4-dioxane (2 mL, 4M) and the mixture was stirred under N₂ atmosphere at room temperature for 2 hours. The reaction mixture was concentrated under reduced pressure to dryness to give the title compound (60 mg, yield 100%) as a white solid, which was used directly in the next step without further purification. LC/MS (ESI) m/z: 165 (M+H)⁺.

### Step 2: 6-Methyl-4,5,6,7-tetrahydrothieno[2, 3-c]pyridine-2-carbonitrile (3)

To a mixture of 4,5,6,7-tetrahydrothieno[2,3-c]pyridine-2-carbonitrile hydrochloride (60 mg, 0.3 mmol) and paraformaldehyde (88 mg, 2.93 mmol) in MeOH (3 mL) was added NaBH₃CN (110 mg, 1.76 mmol), MgSO₄ (100 mg) under N₂ atmosphere and the reaction mixture was stirred at room temperature for 16 hours. The mixture was filtered, and the filtrate was concentrated under reduced pressure to dryness. The residue was purified by flash chromatography (silica gel, 0-50% EtOAc in PE) to give the title compound (50 mg, yield 93.5%) as a colorless oil. LC/MS (ESI) m/z: 179 (M+H)⁺.

### Step 3: (6-Methyl-4,5,6, 7-tetrahydrothieno[2, 3-c]pyridin-2-yl)methanamine (4)

To a solution of 6-methyl-4,5,6,7-tetrahydrothieno[2,3-c]pyridine-2-carbonitrile (30 mg, 0.17 mmol) in EtOAc (3 mL) was added Raney Ni (5 mg), the mixture was degassed under N₂ atmosphere for three times and stirred under a H₂ balloon at room temperature overnight. The mixture was filtered, and the filtrate was concentrated to dryness to give the title compound (145 mg, yield 99%) as a yellow solid, which was used directly in the next step without further purification. LC/MS (ESI) m/z: 183 (M+H)⁺.

### Step 4: (2S,4R)-4-(difluoromethoxy)-N-((6-methyl-4, 5,6,7-tetrahydrothieno[2,3-c]pyridin-2-yl)methyl)-1-((phenoxathiine-3-carbonyl)glycyl)pyrrolidine-2-carboxamide (Compound 346)

To a mixture of (6-methyl-4,5,6,7-tetrahydrothieno[2,3-c]pyridin-2-yl)methanamine (28 mg, 0.15 mmol) and (2S,4R)-4-(difluoromethoxy)-1-((phenoxathiine-3-carbonyl) glycyl)pyrrolidine-2-carboxylic acid (71 mg, 0.15 mmol) in DMF (2 mL) was added DIPEA (99 mg, 0.77 mmol), HATU. (88 mg, 0.23 mmol) and the reaction mixture was stirred at room temperature for 2 hours. The mixture was diluted with EtOAc, washed with brine, dried over anhydrous Na₂SO₄, filtered, and concentrated under reduced pressure to dryness. The residue was purified by prep-TLC (DCM:MeOH=20:1) and further purified by prep-HPLC to give Compound 346 (2.0 mg, yield 2.1%) as a white solid. ¹H NMR (400 MHz, CD₃OD): δ 7.54 (d, J=8.2 Hz, 1H), 7.50 (s, 1H), 7.23-7.18 (m, 2H), 7.15 (d, J=7.2 Hz, 1H), 7.09-7.03 (m, 2H), 6.70 (m, 1H), 6.65-6.31 (m, 1H), 4.99 (s, 1H). 4.54 (s, 2H), 4.46 (m, 2H), 4.19 (d, J=8.8 Hz, 2H), 3.98-3.93 (m, 1H), 3.83 (m, 1H), 3.56 (d, J=7.3 Hz, 2H), 2.72-2.65 (m, 4H), 2.43 (s, 3H), 2.23-2.17 (m, 1H). LC/MS (ESI) m/z: 629 (M+H)⁺. RT (Method A): 1.52 min.

### Scheme 108. Synthesis of (2S,4R)-N-((1H-pyrrolo[3,2-c]pyridin-2-yl)methyl)-4-(difluoromethoxy)-1-((2-fluorophenoxathiine-3-carbonyl)glycyl)pyrrolidine-2-carboxamide (Compound 355)

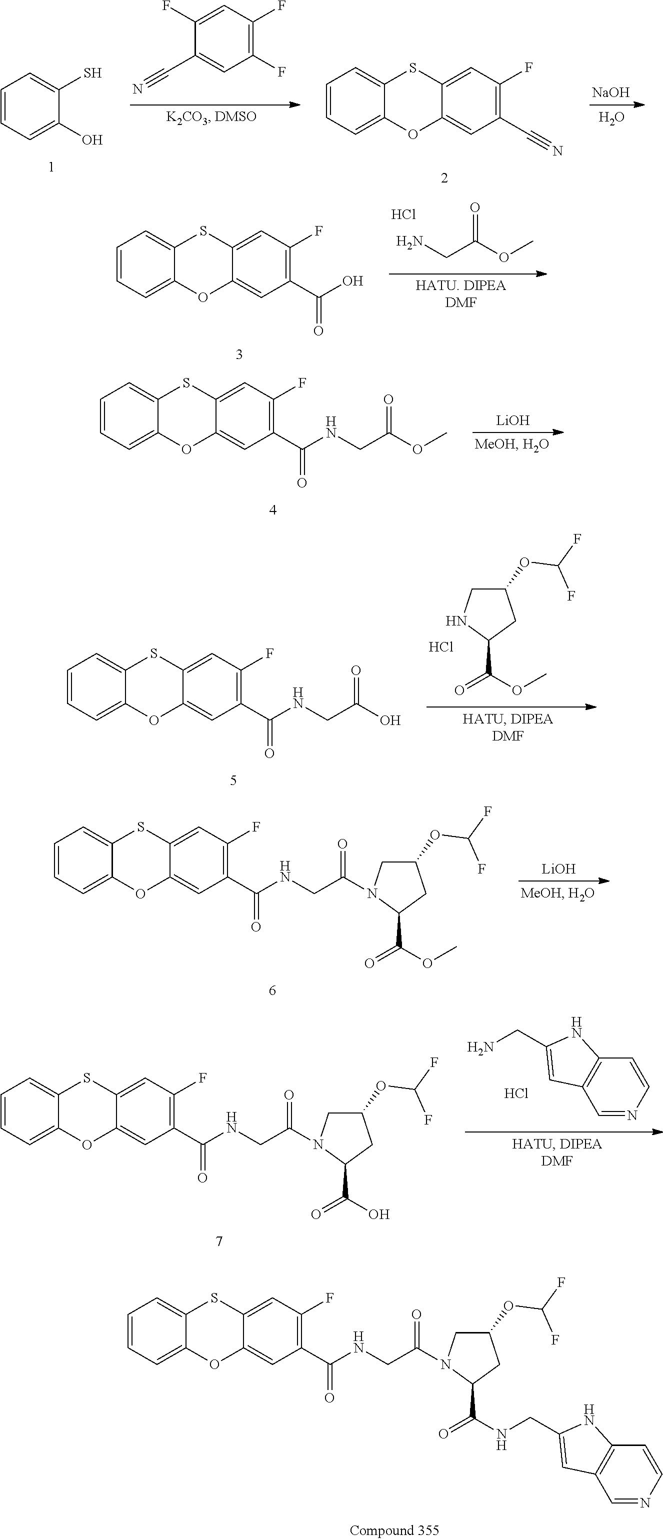

### Step 1: 2-Fluorophenoxathiine-3-carbonitrile (2)

To a mixture of 2-mercaptophenol (300 mg, 2.38 mmol) and 2,4,5-trifluorobenzonitrile (373 mg, 2.38 mmol) in DMSO (5 mL) was added K₂CO₃ (656 mg, 4.76 mmol), the reaction mixture was stirred at 80° C. overnight. The mixture was diluted with EtOAc, washed with brine, dried over anhydrous Na₂SO₄, filtered, and concentrated under reduced pressure to dryness. The residue was purified by flash chromatography (silica gel, 0-9% EtOAc in PE) to give the title compound (150 mg, yield 25.9%) as a white solid. ¹H NMR (400 MHz, CDCl₃) δ 7.43-7.41 (m, 1H), 7.40-7.35 (m, 2H), 7.23 (d, J=7.4 Hz, 1H), 7.17-7.14 (m, 3H), 6.43 (d, J=4.2 Hz, 1H).

### Step 2: 2-Fluorophenoxathiine-3-carboxylic acid (3)

To a solution of 2-fluorophenoxathiine-3-carbonitrile (150 mg, 0.62 mmol) in water (3 mL) was added NaOH (123 mg, 3.07 mmol) and the mixture was stirred at 100° C. overnight. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (120 mg, yield 74.2%) as a yellow solid, which was used directly in the next step without further purification. LC/MS (ESI) m/z: 263 (M+H)⁺.

### Step 3: Methyl (2-fluorophenoxathiine-3-carbonyl)glycinate (4)

To a mixture of 2-fluorophenoxathiine-3-carboxylic acid (100 mg, 0.38 mmol) and methyl glycinate hydrochloride (96 mg, 0.76 mmol) in DMF (3 mL) was added DIPEA (295 mg, 2.29 mmol) and HATU (174 mg, 0.46 mmol), the reaction mixture was stirred at room temperature for 2 hours. The mixture was diluted with EtOAc, washed with brine, dried over anhydrous Na₂SO₄, filtered, and concentrated under reduced pressure to dryness. The residue was purified by flash chromatography (silica gel, 0-25% EtOAc in PE) to give the title compound (95 mg, yield 74.8%) as a white solid. LC/MS (ESI) m/z: 334 (M+H)⁺.

### Step 4: (2-Fluorophenoxathiine-3-carbonyl)glycine (5)

To a solution of methyl (2-fluorophenoxathiine-3-carbonyl)glycinate (95 mg, 0.28 mmol) in MeOH (3 mL) and water (1 mL) was added LiOH (36 mg, 0.85 mmol) and the mixture was stirred at room temperature for 2 hours. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (70 mg, yield 76.9%) as a white solid, which was used directly in the next step without further purification. LC/MS (ESI) m/z: 320 (M+H)⁺.

### Step 5: Methyl (2S,4R)-4-(difluoromethoxy)-1-((2-fluorophenoxathiine-3-carbonyl) glycyl)pyrrolidine-2-carboxylate (6)

To a mixture of (2-fluorophenoxathiine-3-carbonyl)glycine (70 mg, 0.22 mmol) and methyl (2S,4R)-4-(difluoromethoxy)pyrrolidine-2-carboxylate hydrochloride (64 mg, 0.33 mmol) in DMF (3 mL) was added DIPEA (170 mg, 1.32 mmol) and HATU (100 mg, 0.26 mmol) and the reaction mixture was stirred at room temperature for 2 hours. The mixture was diluted with EtOAc, washed with brine, dried over anhydrous Na₂SO₄, filtered, and concentrated under reduced pressure to dryness. The residue was purified by flash chromatography (silica gel, 0-50% EtOAc in PE) to give the title compound (85 mg, yield 78.1%) as a colorless oil. LC/MS (ESI) m/z: 497 (M+H)⁺.

### Step 6: (2S,4R)-4-(difluoromethoxy)-1-((2-fluorophenoxathiine-3-carbonyl)glycyl) pyrrolidine-2-carboxylic acid (7)

To a solution of methyl (2S,4R)-4-(difluoromethoxy)-1-((2-fluorophenoxathiin-3-carbonyl)glycyl)pyrrolidine-2-carboxylate (85 mg, 0.17 mmol) in MeOH (3 mL) and water (1 mL) was added LiOH (22 mg, 0.51 mmol) and the mixture was stirred at room temperature for 2 hours. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (60 mg, yield 72.6%) as a white solid, which was used directly in the next step without further purification. LC/MS (ESI) m/z: 483 (M+H)⁺.

### Step 7: (2S,4R)-N-((1H-pyrrolo[3, 2-c]pyridin-2-yl)methyl)-4-(difluoromethoxy)-1-((2-fluorophenoxathiine-3-carbonyl)glycyl)pyrrolidine-2-carboxamide (Compound 355)

To a mixture of (2S,4R)-4-(difluoromethoxy)-1-((2-fluorophenoxathiine-3-carbonyl) glycyl)pyrrolidine-2-carboxylic acid (60 mg, 0.12 mmol) and (1H-pyrrolo[3,2-c]pyridin-2-yl)methanamine hydrochloride (40 mg, 0.27 mmol) in DMF (2 mL) was added DIPEA (96 mg, 0.72 mmol) and HATU (71 mg, 0.18 mmol) at 0° C., the mixture was stirred at room temperature for 1 hour. The mixture was diluted with EtOAc, washed with water and brine, dried over anhydrous Na₂SO₄, filtered, and concentrated under reduced pressure to dryness. The residue was purified by prep-TLC (DCM:MeOH=10:1) and further purified by prep-HPLC to give Compound 355 (15 mg, yield 19.7%) as a white solid. ¹H NMR (400 MHz, CD₃OD): δ 8.82 (s, 1H), 8.44 (s, 1H), 8.12 (d, J=6.4 Hz, 1H), 7.60 (d, J=6.5 Hz, 1H), 7.35 (d, J=6.3 Hz, 1H), 7.24 (d, J=7.4 Hz, 1H), 7.17 (d, J=7.6 Hz, 1H), 7.12 (s, 1H), 7.10 (d, J=3.1 Hz, 1H), 7.03 (d, J=8.1 Hz, 1H), 6.81 (s, 1H), 6.62 (d, J=74.4 Hz, 1H), 5.05 (s, 1H), 4.71 (s, 1H), 4.64 (d, J=9.0 Hz, 1H), 4.61 (s, 1H), 4.25 (s, 2H), 4.03-3.99 (m, 1H), 3.89 (d, J=11.7 Hz, 1H), 2.55-2.49 (m, 1H), 2.33-2.26 (m, 1H). LC/MS (ESI) (m/z): 612 (M+H)⁺. RT (Method A): 1.50 min.

Compounds 352-354 can be prepared based on the procedures shown in Scheme 107 with the following reactants:

| Cmpd # | Reactant A | Reactant B | Reactant C | Reactant D | Reactant E |
|---|---|---|---|---|---|
| 352 | 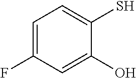 | 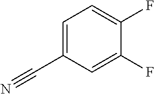 | 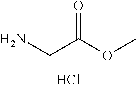 | 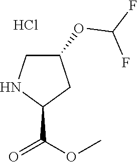 | 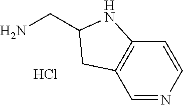 [SMILES: NCC1Cc2cnccc2N1 ; Cl] |
| 353 | 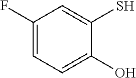 | 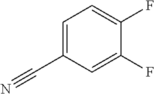 | 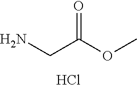 | 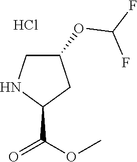 | 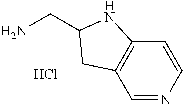 [SMILES: NCC1Cc2cnccc2N1 ; Cl] |
| 354 | 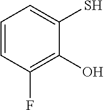 | 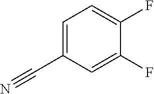 | 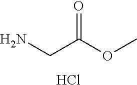 | 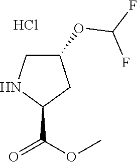 | 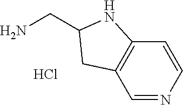 [SMILES: NCC1Cc2cnccc2N1 ; Cl] |

### Scheme 109. Synthesis of (2S,4R)-N-((1H-pyrrolo[3,2-c]pyridin-2-yl)methyl)-4-(difluoromethoxy)-1-((2,3-dihydrobenzo[b][1,4]oxathiine-7-carbonyl)glycyl)pyrrolidine-2-carboxamide (Compound 356)

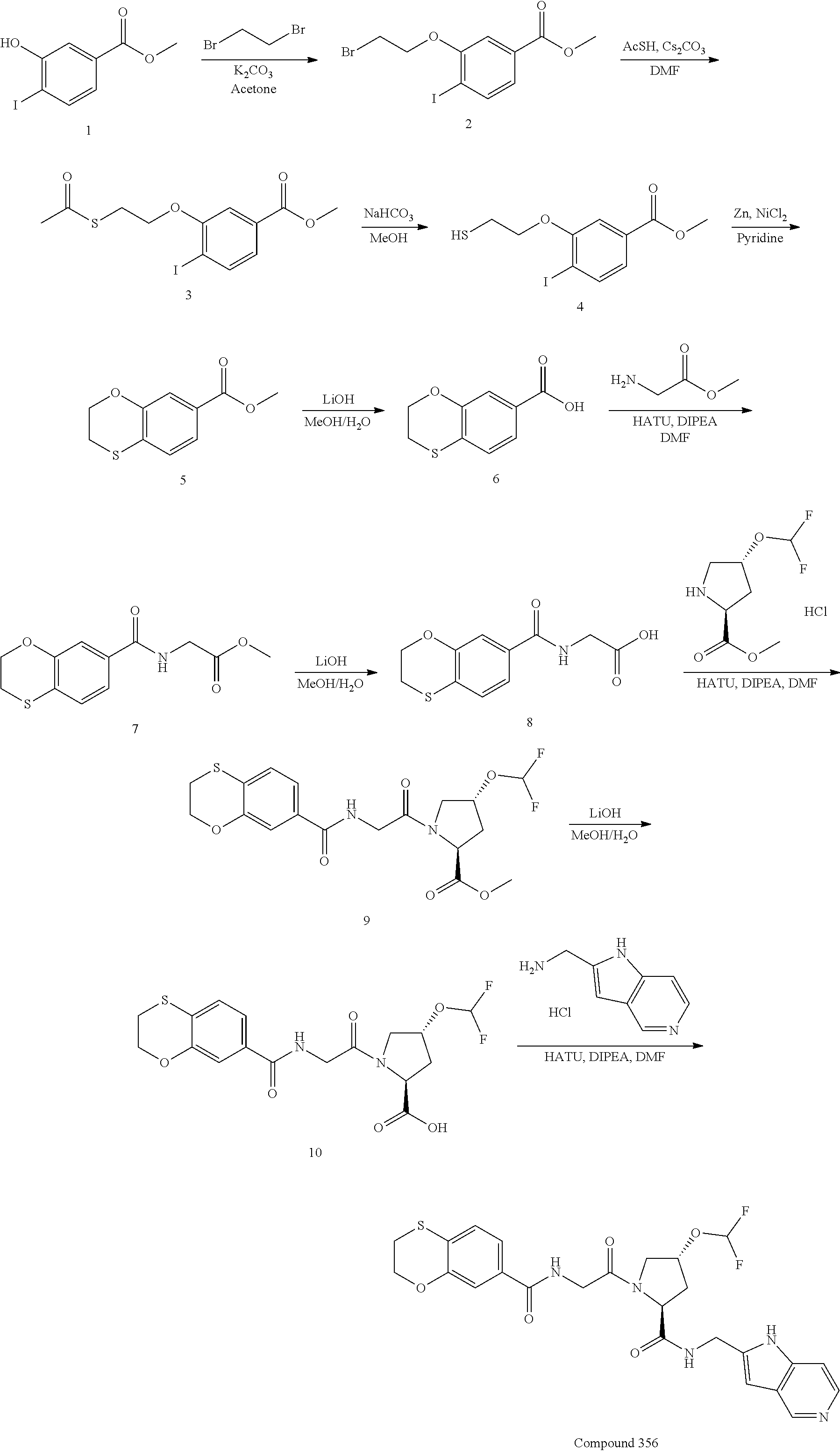

### Step 1: Methyl 3-(2-bromoethoxy)-4-iodobenzoate (2)

To a solution of methyl 3-hydroxy-4-iodobenzoate (4.22 g, 15.18 mmol) in acetone (5 mL) was added K₂CO₃ (4.2 g, 30.36 mmol) under N₂ atmosphere and the reaction mixture was stirred at room temperature for 0.5 hour. Then 1,2-dibromoethane (15.26 g, 75.90 mmol) was added to the mixture and the reaction mixture was stirred at 40° C. overnight. The mixture was diluted with EtOAc, washed with brine, dried over anhydrous Na₂SO₄, filtered, and concentrated under reduced pressure to dryness. The residue was purified by flash chromatography (silica gel, 0-50% EtOAc in PE) to give the title compound (3.4 g, yield 58.6%) as a white solid. LC/MS (ESI) m/z: 385 (M+H)⁺.

### Step 2: Methyl 3-(2-(acetylthio)ethoxy)-4-iodobenzoate (3)

To a solution of AcSH (773 mg, 9.38 mmol) in DMF (20 mL) was added Cs₂CO₃ (3.31 g, 9.38 mmol) under N₂ atmosphere and the reaction mixture was stirred at room temperature for 0.5 hour. Then methyl 3-(2-bromoethoxy)-4-iodobenzoate (2.6 g, 6.25 mmol) was added to the mixture under N₂ atmosphere and the reaction mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (1.18 g, yield 46.1%) as a white solid. LC/MS (ESI) m/z: 381 (M+H)⁺.

### Step 3: Methyl 4-iodo-3-(2-mercaptoethoxy)benzoate (4)

To a solution of methyl 3-(2-(acetylthio)ethoxy)-4-iodobenzoate (500 mg, 1.32 mmol) in MeOH (5 mL) was added NaHCO₃ (166 mg, 1.97 mmol) under N₂ atmosphere at 0° C. and the reaction mixture was stirred at room temperature for 2 hours. The mixture was diluted with EtOAc, washed with brine, dried over anhydrous Na₂SO₄, filtered, and concentrated under reduced pressure to dryness. The residue was purified by flash chromatography (silica gel, 0-30% EtOAc in PE) to give the title compound (300 mg, yield 67.4%) as a white solid. LC/MS (ESI) m/z: 339 (M+H)⁺.

### Step 4: Methyl 2,3-dihydrobenzo[b][1,4]oxathiine-7-carboxylate (5)

To a mixture of Zn (81 mg, 1.24 mmol) and NiCl₂ (41 mg, 0.31 mmol) in pyridine (3 mL) was added 2,2′-bipyridine (59 mg, 0.37 mmol) under N₂ atmosphere and the reaction mixture was stirred at 55° C. for 0.5 hour. Then the mixture was cooled to room temperature and methyl 4-iodo-3-(2-mercaptoethoxy) benzoate (210 mg, 0.62 mmol) was added to the mixture under N₂ atmosphere. The reaction mixture was stirred at room temperature for 3 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (60 mug, yield 46.2%) as a white solid. LC/MS (ESI) m/z: 211 (M+H)⁺.

### Step 5: 2, 3-Dihydrobenzo[b][1, 4]oxathiine-7-carboxylic acid (6)

To a solution of methyl 2,3-dihydrobenzo[b][1,4]oxathiine-7-carboxylate (60 mg, 0.29 mmol) in MeOH (2 mL) and water (1 mL) was added LiOH (18 mg, 0.43 mmol) and the mixture was stirred at room temperature for 5 hours. The mixture was acidified with 1N aq·HCl to pH˜3 and extracted with EtOAc twice. The combined organic layers were dried over anhydrous Na₂SO₄, filtered, and concentrated under reduced pressure to dryness to give the title compound (50 mg, yield 89.2%) as a white solid, which was used directly in the next step without further purification. LC/MS (ESI) m/z: 197 (M+H)⁺.

### Step 6: Methyl (2,3-dihydrobenzo[b][1,4]oxathiine-7-carbonyl)glycinate (7)

To a solution of 2,3-dihydrobenzo[b][1,4]oxathiine-7-carboxylic acid (50 mg, 0.31 mmol) in DMF (2 mL) was added methyl glycinate (75 mg. 0.46 mmol), HATU (175 mg, 0.46 mmol) and DIPEA (119 mg, 0.92 mmol) and the mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give the title compound (33 mg, yield 40.2%) as a white solid. LC/MS (ESI) m/z: 268 (M+H)⁺.

### Step 7: (2,3-Dihydrobenzo[b][1,4]oxathiine-7-carbonyl)glycine (8)

To a solution of methyl (2,3-dihydrobenzo[b][1,4]oxathiine-7-carbonyl)glycinate (33 mg, 0.12 mmol) in MeOH (2 mL) and water (1 mL) was added LiOH (8 mg, 0.18 mmol) and the mixture was stirred at room temperature for 5 hours. The mixture was acidified with 1N aq·HCl to pH˜-3 and extracted with EtOAc twice. The combined organic layers were concentrated under reduced pressure to dryness to give the title compound (30 mg, yield 96.8%) as a white solid, which was used directly in the next step without further purification. LC/MS (ESI) m/z: 254 (M+H)⁺.

### Step 8: Methyl (2S,4R)-4-(difluoromethoxy)-1-((2, 3-dihydrobenzo[b][1,4]oxathiane-7-carbonyl)glycyl)pyrrolidine-2-carboxylate (9)

To a solution of (2,3-dihydrobenzo[b][1,4]oxathiine-7-carbonyl)glycine (30 mg, 0.12 mmol) in DMF (2 mL) was added methyl (2S,4R)-4-(difluoromethoxy)pyrrolidine-2-carboxylate hydrochloride (28 mg, 0.14 mmol), HATU (68 mg, 0.18 mmol) and DIPEA (46 mg, 0.36 mmol). The mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) to give the title compound (15 mg, yield 51.0%) as a white solid. LC/MS (ESI) m/z: 431 (M+H)⁺.

### Step 9: (2S,4R)-4-(difluoromethoxy)-1-((2,3-dihydrobenzo[b][1,4]oxathiine-7-carbonyl)glycyl)pyrrolidine-2-carboxylic acid (10)

To a solution of methyl (2S,4R)-4-(difluoromethoxy)-1-((2,3-dihydrobenzo[b][1,4]oxathiine-7-carbonyl)glycyl)pyrrolidine-2-carboxylate (15 mg, 0.03 mmol) in MeOH (2 mL) and water (1 mL) was added LiOH (3 mg, 0.05 mmol) and the mixture was stirred at room temperature for 5 hours. The mixture was acidified with 1N aq·HCl to pH˜3 and extracted with EtOAc twice. The reaction mixture was concentrated under reduced pressure to dryness to give the title compound (12 mg, yield 82.8%) as a white solid, which was used directly in the next step without further purification. LC/MS (ESI) m/z: 417 (M+H)⁺.

### Step 10: (2S,4R)-N-((1H-pyrrolo[3, 2-c]pyridin-2-yl)methyl)-4-(difluoromethoxy)-1-((2,3-dihydrobenzo[b][1,4]oxathiine-7-carbonyl)glycyl)pyrrolidine-2-carboxamide (Compound 356)

To a solution of (2S,4R)-4-(difluoromethoxy)-1-((2,3-dihydrobenzo[b][1,4]oxathiine-7-carbonyl)glycyl)pyrrolidine-2-carboxylic acid (12 mg, 0.03 mmol) in DMF (2 mL) was added (1H-pyrrolo[3,2-c]pyridin-2-yl)methanamine hydrochloride (6 mg, 0.03 mmol), HATU (17 mg, 0.05 mmol) and DIPEA (12 mg, 0.09 mmol) and the mixture was stirred at room temperature overnight. The mixture was diluted with EtOAc, washed with water and brine, dried over anhydrous Na₂SO₄, filtered, and concentrated under reduced pressure to dryness. The residue was purified by flash chromatography (silica gel, 0-10% MeOH in DCM) and further purified by prep-HPLC to give Compound 356 (0.8 mg, yield 5.10%) as a white solid. ¹H NMR (400 MHz, CD₃OD): δ 8.63 (d, J=0.8 Hz, 1H), 7.98 (d, J=5.8 Hz. 1H), 7.39-7.27 (m, 3H), 7.16 (d, J=5.8 Hz, 1H), 7.10 (d, J=8.1 Hz, 1H), 6.51 (d, J=9.5 Hz, 2H), 5.04 (s, 1H), 4.68 (s, 1H), 4.59 (d, J=8.1 Hz, 1H), 4.55 (d, J=4.0 Hz, 1H), 4.45-4.38 (m, 3H), 4.24 (m, 1H), 4.16 (m, 1H), 4.01-3.95 (m, 1H), 3.88 (m, 1H), 3.25-3.16 (m, 3H), 3.01 (s, 2H). 2.54-2.44 (m, 1H), 2.32-2.23 (m, 1H). LC/MS (ESI) m/z: 546 (M+H)⁺. RT (Method A): 0.91 min.

### Scheme 110. Synthesis of (S)-N-((1H-pyrrolo[3,2-c]pyridin-2-yl)methyl)-1-((6-methyldibenzo[b,d]furan-2-carbonyl)glycyl)pyrrolidine-2-carboxamide (Compound 336)

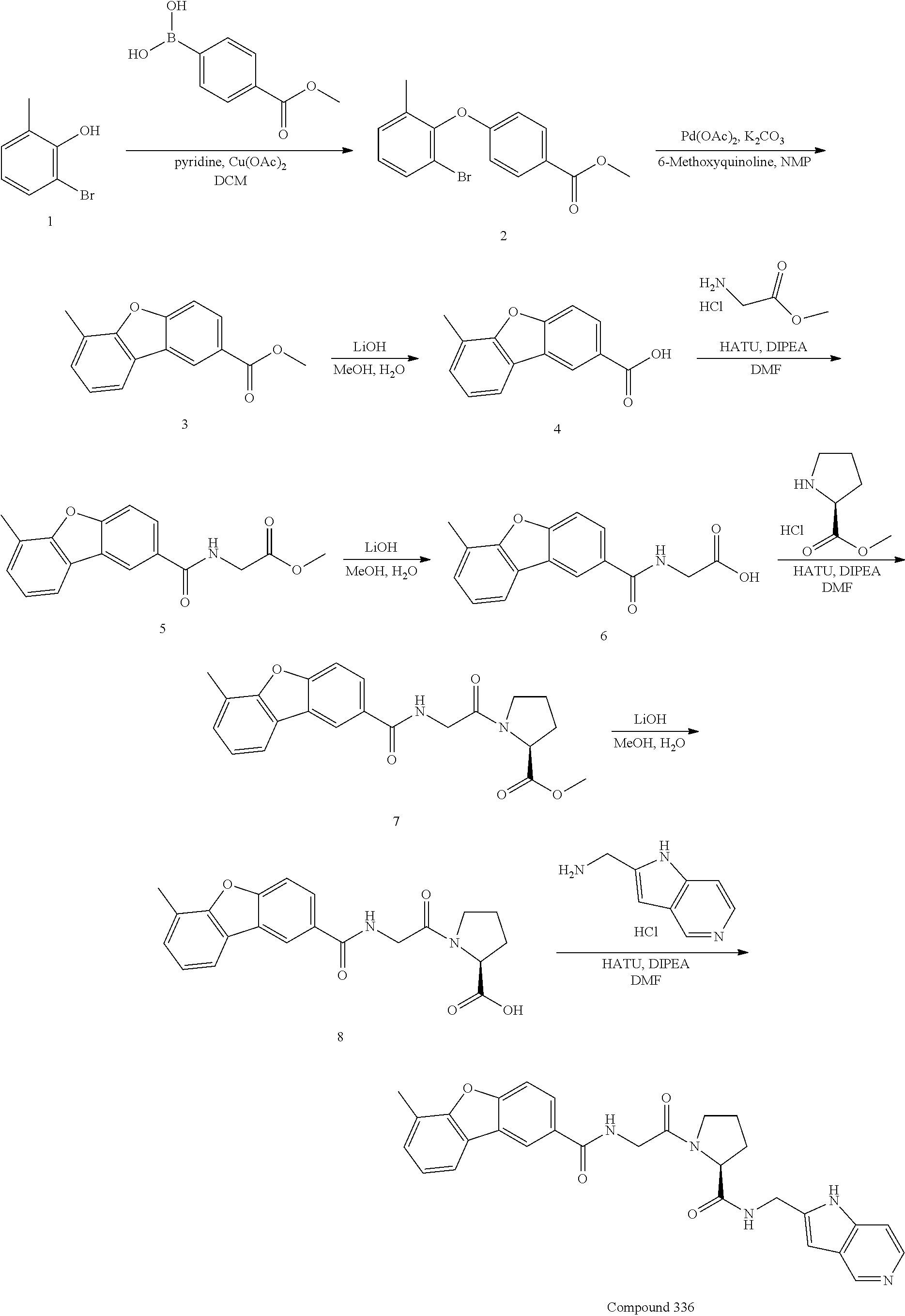

### Step 1: Methyl 4-(2-bromo-6-methylphenoxy)benzoate (2)

To a mixture of 2-bromo-6-methylphenol (500 mg, 2.67 mmol) and (4-(methoxycarbonyl)phenyl)boronic acid (962 mg, 5.35 mmol) in dry DCM (10 mL) was added pyridine (423 mg, 5.35 mmol), Cu(OAc)₂ (728 mg, 4.01 mmol), the reaction mixture was degassed under 02 atmosphere for three times and stirred at room temperature overnight. The mixture was filtered, and the filtrate was concentrated under reduced pressure to dryness. The residue was purified by flash chromatography (silica gel, 0-5% EtOAc in PE) to give the title compound (670 mg, yield 78.0%) as a colorless oil. ¹H NMR (400 MHz, CDCl₃) δ 7.98 (d, J=8.2 Hz, 2H), 7.49 (d, J=8.0 Hz, 1H), 7.23 (d, J=7.5 Hz, 1H), 7.06 (t, J=7.8 Hz, 1H), 6.81 (d, J=8.2 Hz, 2H), 3.88 (s, 3H), 2.17 (s, 3H).

### Step 2: Methyl 6-methyldibenzo[b,d]furan-2-carboxylate (3)

To a solution of methyl 4-(2-bromo-6-methylphenoxy)benzoate (570 mg, 1.77 mmol) in NMP (15 mL) was added Pd(OAc)₂ (40 mg, 0.18 mmol), 6-methoxyquinoline (57 mg, 0.35 mmol) and K₂CO₃ (491 mg, 3.55 mmol) at 25° C. under N₂ atmosphere, the reaction mixture was degassed under N₂ atmosphere for three times and stirred under N₂ atmosphere at 135° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-7% EtOAc in PE) to give the title compound (230 mg, yield 54.0%) as a colorless oil. LC/MS (ESI) m/z: 241 (M+H)⁺.

### Step 3: 6-Methyldibenzo[b,d]furan-2-carboxylic acid (4)

To a solution of methyl 6-methyldibenzo[b,d]furan-2-carboxylate (230 mg, 0.96 mmol) in MeOH (2 mL) and water (1 mL) was added LiOH (46 mg, 1.91 mmol) and the mixture was stirred at room temperature for 1 hour. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (215 mg, yield 99.3%) as a white solid, which was used directly in the next step without further purification. LC/MS (ESI) m/z: 227 (M+H)⁺.

### Step 4: Methyl (6-methyldibenzo[b,d]furan-2-carbonyl)glycinate (5)

To a mixture of 6-methyldibenzo[b,d]furan-2-carboxylic acid (100 mg, 0.44 mol) and methyl glycinate hydrochloride (83 mg, 0.66 mmol) in DMF (5 mL) was added DIPEA (343 mg, 2.65 mmol) and HATU (185 mg, 0.49 mol) and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel. 0-25% EtOAc in PE) to give the title compound (130 mg, yield 98.9%) as a colorless oil. LC/MS (ESI) m/z: 298 (M+H)⁺.

### Step 5: (6-Methyldibenzo[b,d]furan-2-carbonyl)glycine (6)

To a solution of methyl (6-methyldibenzo[b,d]furan-2-carbonyl)glycinate (130 mg, 0.44 mol) in MeOH (2 mL) and water (1 mL) was added LiOH (21 mg, 0.87 mol) and the mixture was stirred at room temperature for 1 hour. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (120 mg, yield 96.9%) as a white solid, which was used directly in the next step without further purification. LC/MS (ESI) m/z: 284 (M+H)⁺.

### Step 6: Methyl (6-methyldibenzo[b,d]furan-2-carbonyl)glycyl-L-prolinate (7)

To a mixture of (6-methyldibenzo[b,d]furan-2-carbonyl)glycine (60 mg, 0.21 mol) and methyl L-prolinate hydrochloride (35 mg, 0.21 mmol) in DMF (3 mL) was added DIPEA (164 mg, 1.27 mmol) and HATU (89 mg, 0.23 mol). The mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (60 mg, yield 71.8%) as a yellow oil. LC/MS (ESI) m/z: 395 (M+H)⁺.

### Step 7: (6-Methyldibenzo[b,d]furan-2-carbonyl)glycyl-L-proline (8)

To a solution of methyl (6-methyldibenzo[b,d]furan-2-carbonyl)glycyl-L-prolinate (60 mg, 0.15 mol) in MeOH (2 mL) and water (1 mL) was added LiOH (7 mg, 0.30 mol) and the mixture was stirred at room temperature for 1 hour. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (50 mg, yield 86.4%) as a yellow oil, which was used directly in the next step without further purification. LC/MS (ESI) m/z: 381 (M+H)⁺.

### Step 8: (S)-N-((1H-pyrrolo[3,2-c]pyridin-2-yl)methyl)-1-((6-methyldibenzo[b,d]furan-2-carbonyl)glycyl)pyrrolidine-2-carboxamide (Compound 336)

To a mixture of (6-methyldibenzo[b,d]furan-2-carbonyl)glycyl-L-proline (50 mg, 0.13 mol) and (1H-pyrrolo[3,2-c]pyridin-2-yl)methanamine hydrochloride (36 mg, 0.20 mmol) in DMF (5 mL) was added DIPEA (102 mg, 0.79 mmol) and HATU (55 mg, 0.14 mol) and the mixture was stirred at room temperature for 1 hour. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=8:1) and further purified by prep-HPLC to give Compound 336 (8 mg, yield 11.9%) as a white solid. ¹H NMR (400 MHz, CD₃OD): δ 8.70-8.44 (m, 2H), 8.07-7.94 (m, 1H), 7.83 (m, 2H), 7.61 (t, J=7.9 Hz, 1H), 7.31 (m, 2H), 7.17 (d, J=5.6 Hz, 1H), 6.53 (m, 1H), 4.62 (s, 2H), 4.52 (dd, J=8.5, 3.8 Hz, 1H), 4.28 (d, J=2.3 Hz, 2H), 3.79 (m, 2H), 2.59 (s, 3H), 2.29 (s, 1H), 2.14 (m, 3H). LC/MS (ESI) m/z: 510 (M+H)⁺. RT (Method A): 1.25 min.

### Scheme 111. Synthesis of (2S,4R)-N-((1H-pyrrolo[3,2-c]pyridin-2-yl)methyl)-4-(difluoromethoxy)-1-((7-methyldibenzo[b,d]furan-2-carbonyl)glycyl)pyrrolidine-2-carboxamide (Compound 337)

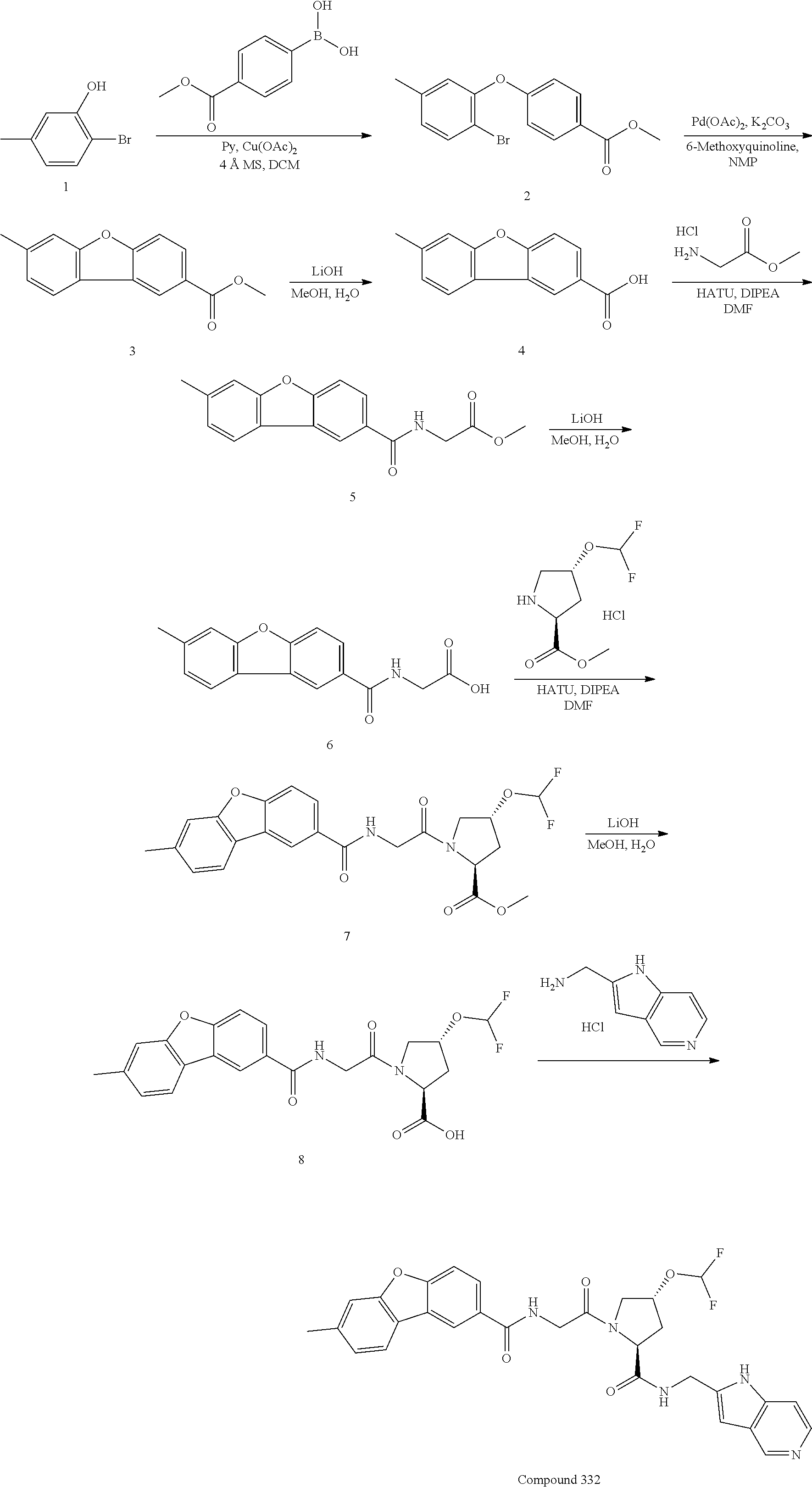

### Step 1: Methyl 4-(2-bromo-5-methylphenoxy)benzoate (2)

To a mixture of 2-bromo-5-methylphenol (500 mg, 2.67 mmol) and (4-(methoxy carbonyl)phenyl)boronic acid (721 mg, 4.01 mmol) in DCM (5 mL) was added pyridine (422 mg, 5.35 mmol), Cu(OAC)₂ (729 mg, 4.01 mmol) and 4kA MS (2 g) at 0° C. under N₂ atmosphere and the mixture was stirred at room temperature for 2 hours. The mixture was diluted with DCM and filtered. The filtrate was washed with saturated aq·NaHCO₃ solution, dried over anhydrous Na₂SO₄, filtered, and concentrated under reduced pressure to dryness. The residue was purified by flash chromatography (silica gel, 0-7% EtOAc in PE) to give the title compound (450 mg, yield 52.6%) as a yellow oil. LC/MS (ESI) m/z: 321 (M+H)⁺.

### Step 2: Methyl 7-methyldibenzo[b,d]furan-2-carboxylate (3)

To a solution of methyl 4-(2-bromo-5-methylphenoxy)benzoate (380 mg, 1.19 mmol) in NMP (4 mL) was added Pd(OAc)₂ (27 mg, 0.12 mmol), 6-Methoxyquinoline (38 mg, 0.24 mmol) and K₂CO₃ (330 mg, 2.38 mmol) at 25° C. under N₂ atmosphere, the reaction mixture was degassed under N₂ atmosphere for three times and stirred under N₂ atmosphere at 135° C. overnight. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-10% EtOAc in PE) to give the title compound (190 mg, yield 66.7%) as a colorless oil. LC/MS (ESI) m/z: 241 (M+H)⁺.

### Step 3: 7-Mhyldibenzo[b,d]furan-2-carboxylic acid (4)

To a solution of methyl 7-methyldibenzo[b,d]furan-2-carboxylate (190 mg, 0.79 mmol) in MeOH (2 mL) and water (1 mL) was added LiOH (50 mg, 1.19 mmol), the mixture was stirred at room temperature for 1 hour. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (110 mg, yield 90.1%) as a white solid, which was used directly in the next step without further purification. LC/MS (ESI) m/z: 227 (M+H)⁺.

### Step 4: Methyl (7-methyldibenzo[b,d]furan-2-carbonyl)glycinate (5)

To a mixture of 7-methyldibenzo[b,d]furan-2-carboxylic acid (110 mg, 0.58 mmol) and methyl glycinate hydrochloride (109 mg, 0.86 mmol) in DMF (3 mL) was added DIPEA (223 mg, 1.73 mmol), HATU (328 mg, 0.86 mmol) and the mixture was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-33% EtOAc in PE) to give the title compound (120 mg, yield 82.8%) as a colorless oil. LC/MS (ESI) m/z: 298 (M+H)⁺.

### Step 5: (7-Methyldibenzo[b,d]furan-2-carbonyl)glycine (6)

To a solution of methyl (7-methyldibenzo[b,d]furan-2-carbonyl)glycinate (120 mg, 0.40 mmol) in MeOH (2 mL) and water (1 mL) was added LiOH (25 mg, 0.60 mmol) and the mixture was stirred at room temperature for 1 hour. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (110 mg, yield 96.5%) as a white solid, which was used directly in the next step without further purification. LC/MS (ESI) m/z: 284 (M+H)⁺.

### Step 6: Methyl (2S,4R)-4-(difluoromethoxy)-1-((7-methyldibenzo[b,d]furan-2-carbonyl)glycyl)pyrrolidine-2-carboxylate (7)

To a mixture of (7-methyldibenzo[b,d]furan-2-carbonyl)glycine (110 mg, 0.39 mmol) and methyl (2S,4R)-4-(difluoromethoxy)pyrrolidine-2-carboxylate hydrochloride (125 mg, 0.64 nmol) in DMF (2 mL) was added DIPEA (205 mg, 1.59 mmol) and HATU (303 mg, 0.80 mmol). The mixture was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-50% EtOAc in PE) to give the title compound (160 mg, yield 89.4%) as a yellow oil. LC/MS (ESI) m/z: 461 (M+H)⁺.

### Step 7: (2S,4R)-4-(difluoromethoxy)-1-((7-methyldibenzo[b,d]furan-2-carbonyl) glycyl)pyrrolidine-2-carboxylic acid (8)

To a solution of methyl (2S,4R)-4-(difluoromethoxy)-1-((7-methyldibenzo[b,d]furan-2-carbonyl)glycyl)pyrrolidine-2-carboxylate (160 mg, 0.35 mmol) in MeOH (2 mL) and water (1 mL) was added LiOH (22 mg, 0.52 mmol), and the mixture was stirred at room temperature for 1 hour. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (100 mg, yield 64.5%) as a yellow oil, which was used directly in the next step without further purification. LC/MS (ESI) n/z: 447 (M+H)⁺.

### Step 8: (2S,4R)-N-((1H-pyrrolo[3, 2-c]pyridin-2-yl)methyl)-4-(difluoromethoxy)-1-((7-methyldibenzo[b,d]furan-2-carbonyl)glycyl)pyrrolidine-2-carboxamide (Compound 332)

To a mixture of (2S,4R)-4-(difluoromethoxy)-1-((7-methyldibenzo[b,d]furan-2-carbonyl)glycyl)pyrrolidine-2-carboxylic acid (100 mg, 0.22 mmol) and (LH-pyrrolo[3,2-c]pyridin-2-yl)methanamine hydrochloride (62 mg, 0.34 mmol) in DMF (3 mL) was added DIPEA (87 mg, 0.67 mmol), HATU (128 mg, 0.34 mmol) and the mixture was stirred at room temperature for 2 hours. The mixture was diluted with water and extracted with EtOAc twice. The combined organic layers were washed with brine, dried over anhydrous Na₂SO₄, filtered, and concentrated under reduced pressure to dryness. The residue was purified by prep-TLC (DCM:MeOH=20:1) and further purified by prep-HPLC to give Compound 332 (1 mg, yield 0.8%) as a white solid. ¹H NMR (400 MHz, CD₃OD): δ 8.66 (s, 1H), 8.50 (d, J=5.1 Hz, 1H), 8.43 (d, J=1.6 Hz, 1H), 7.94 (m, 1H), 7.86 (m, 2H), 7.57 (d, J=8.6 Hz, 1H), 7.45 (s, 1H), 7.38 (d, J=6.3 Hz, 1H), 7.25 (d, J=7.9 Hz, 1H), 6.71 (s, 1H), 6.52 (m, 1H), 5.08-5.06 (m, 1H), 4.71-4.69 (m, 1H), 4.62 (m, 2H), 4.28 (s, 2H), 4.07-4.04 (m, III), 3.96-3.93 (m, 1H), 2.54 (s, 3H), 2.51-2.49 (m, 1H), 2.34-2.27 (m, III). LC/MS (ESI) m/z: 576 (M+H)⁺. RT (Method A): 1.46 min.

Compounds 330, 331, 333, 335, and 337 can be prepared based on Scheme 110 with the following reactants:

| Cmpd | | | | | |
|---|---|---|---|---|---|
| # | Reactant A | Reactant B | Reactant C | Reactant D | Reactant E |
| 330 | 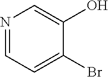 | 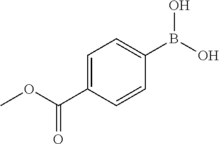 | 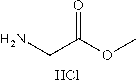 | 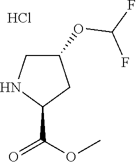 | 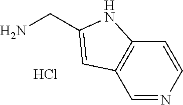 [SMILES: NCc1cc2cnccc2[nH]1 ; Cl] |
| 331 | 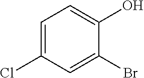 | 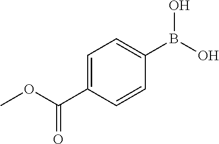 | 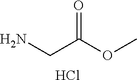 | 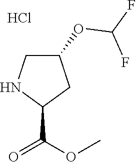 | 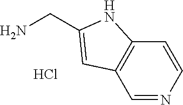 [SMILES: NCc1cc2cnccc2[nH]1 ; Cl] |
| 333 | 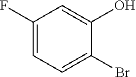 | 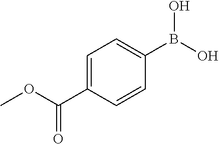 | 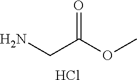 | 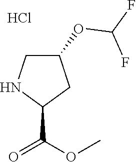 | 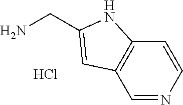 [SMILES: NCc1cc2cnccc2[nH]1 ; Cl] |
| 335 | 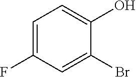 | 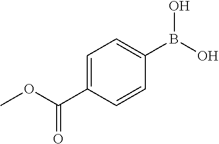 | 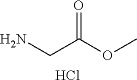 | 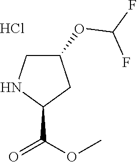 | 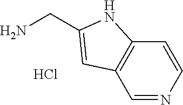 [SMILES: NCc1cc2cnccc2[nH]1 ; Cl] |
| 337 | 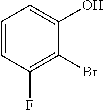 | 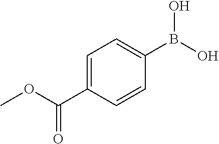 | 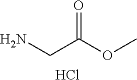 | 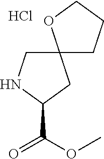 | 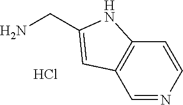 [SMILES: NCc1cc2cnccc2[nH]1 ; Cl] |

### Scheme 112. Synthesis of (S)-N-((1H-pyrrolo[3,2-c]pyridin-2-yl)methyl)-1-((2-fluoro-3-(p-tolyloxy)benzoyl)glycyl)-4,5-dihydro-1H-pyrazole-5-carboxamide (Compound 375)

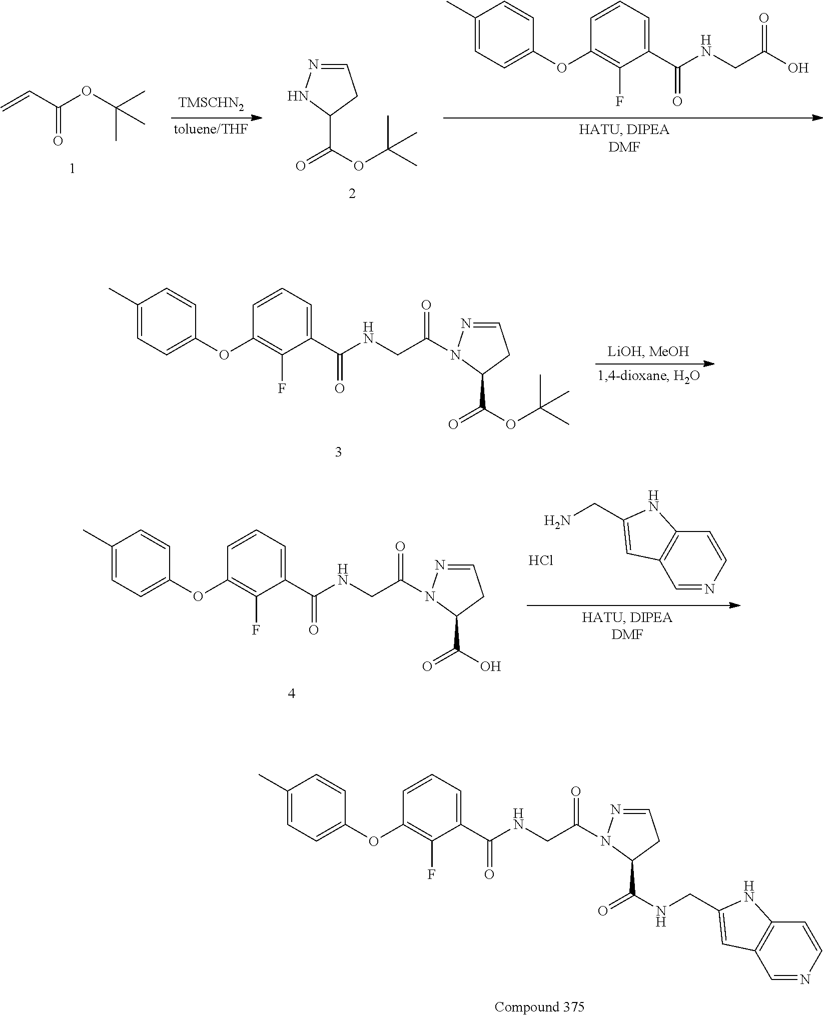

### Step 1: Tert-butyl (S)-4, 5-dihydro-1H-pyrazole-5-carboxylate (2)

To a mixture of tert-butyl acrylate (500 mg, 3.91 mmol) in THF (3 mL) and toluene (3 ml) was added TMS-diazomethane (2.35 mL, 4.7 mmol, 2M in hexane). The reaction mixture was degassed under N₂ atmosphere for three times and stirred at room temperature for 4 hours. The mixture was diluted with water and extracted with EtOAc twice. The combined organic layers were washed with saturated aq·Na₂CO₃ solution and brine, dried over anhydrous Na₂SO₄, filtered, and concentrated under reduced pressure to dryness. The residue was purified by flash chromatography (silica gel, 0-20% EtOAc in PE) to give the title compound (130 mg, yield 19.6%) as a yellow oil. LC/MS (ESI) m/z: 171 (M+H)⁺.

### Step 2: Tert-butyl (S)-1-((2-fluoro-3-(p-tolyloxy)benzoyl)glycyl)-4,5-dihydro-1H-pyrazole-5-carboxylate (3)

To a mixture of (2-fluoro-3-(p-tolyloxy)benzoyl)glycine (45 mg, 0.15 mmol) and Tert-butyl (S)-4,5-dihydro-1H-pyrazole-5-carboxylate (30 mg, 0.18 mmol) in DMF (2 mL) was added HATU (65 mg, 0.17 mmol), DIPEA (69 mg, 0.53 mmol) and the mixture was stirred at room temperature for 0.5 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-20% EtOAc in PE) to give the title compound (45 mg, yield 60.7%) as a yellow oil. LC/MS LC/MS (ESI) m/z: 456 (M+H)⁺.

### Step 3: (S)-1-((2-fluoro-3-(p-tolyloxy)benzoyl)glycyl)-4,5-dihydro-1H-pyrazole-5-carboxylic acid (4)

To a solution of tert-butyl (S)-1-((2-fluoro-3-(p-tolyloxy)benzoyl)glycyl)-4,5-dihydro-1H-pyrazole-5-carboxylate (45 mg, 0.10 mmol) in 1,4-dioxane (1 mL), MeOH (1 ml.) and water (1 mL) was added LiOH (13 mg, 0.54 mmol) and the mixture was stirred at 60° C. for 2 hours. The mixture was acidified with 1N aq·HCl to pH˜4 and extracted with EtOAc twice. The combined organic layers were washed with brine, dried over anhydrous Na₂SO₄, filtered, and concentrated under reduced pressure to dryness to give the title compound (32 mg, yield 80.0%) as a yellow oil. LC/MS (ESI) m/z: 400 (M+H)⁺.

### Step 4: (S)-N-((1H-pyrrolo[3,2-c]pyridin-2-yl)methyl)-1-((2-fluoro-3-(p-tolyloxy) benzoyl)glycyl)-4,5-dihydro-1H-pyrazole-5-carboxamide (Compound 375)

To a mixture of (S)-1-((2-fluoro-3-(p-tolyloxy)benzoyl)glycyl)-4,5-dihydro-1H-pyrazole-5-carboxylic acid (32 mg, 0.08 mmol) and (1H-pyrrolo[3,2-c]pyridin-2-yl)methanamine hydrochloride (20 mg, 0.11 mmol) in DMF (2 mL) was added HATU (37 mg, 0.10 mmol), DIPEA (42 mg, 0.32 mmol) and the mixture was stirred at room temperature for 0.5 hour. The mixture was diluted with water and extracted with EtOAc twice. The combined organic layers were washed with brine, dried over anhydrous Na₂SO₄, filtered, and concentrated under reduced pressure to dryness. The residue was purified by flash chromatography (silica gel, 0-13% MeOH in DCM) and further purified by prep-HPLC to give Compound 375 (2.3 mg, yield 5.3%) as a white solid. ¹H NMR (400 MHz, DMSO-d6) δ 8.80-8.74 (m, 2H), 8.57 (s, 1H), 8.13-8.09 (m. 1H), 7.47-7.42 (m, 1H), 7.34 (s, 1H), 7.29-7.24 (m, 3H), 7.24-7.16 (m, 3H), 6.94-6.91 (m, 2H), 6.46 (s, 1H), 4.76-4.71 (m, 1H), 4.49-4.41 (m, 3H), 4.32-4.26 (m, 1H), 3.26 (s, 1H), 2.97-2.90 (m, 1H), 2.28 (s, 3H). LC/MS (ESI) m/z: 529 (M+H)⁺. RT (Method A): 1.31 min.

### Scheme 113. Synthesis of (2S,4R)-4-(difluoromethoxy)-1-((2-fluoro-3-(p-tolyloxy)benzoyl)glycyl)-N-(thieno[3,2-c]pyridin-2-yl)pyrrolidine-2-carboxamide (Compound 376)

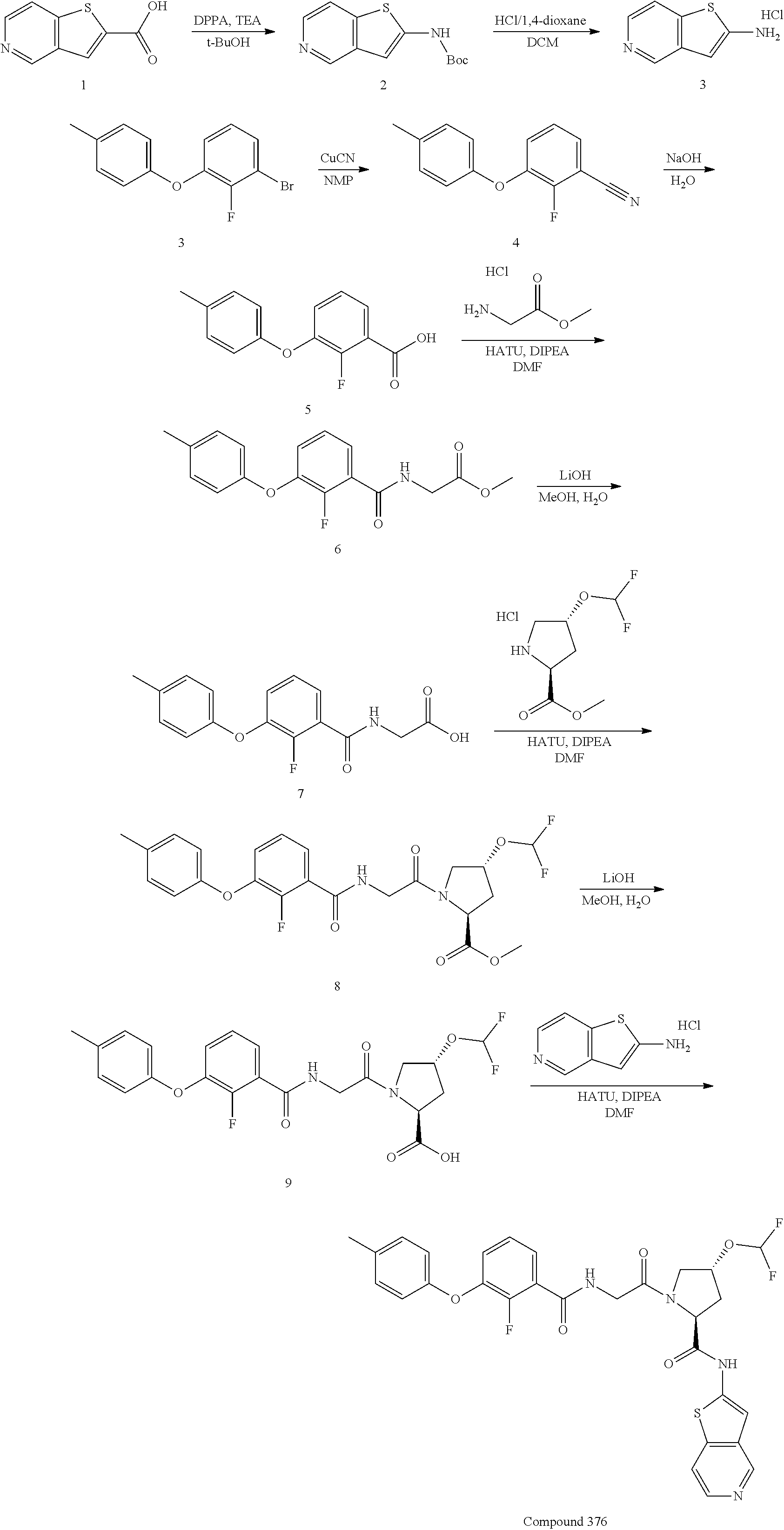

### Step 1: Tert-butyl thieno[3,2-c]pyridin-2-ylcarbamate (2)

To a solution of thieno[3,2-c]pyridine-2-carboxylic acid (500 mg, 2.79 mmol) in t-BuOH (5 mL) was added TEA (846 mg, 8.38 mmol) and DPPA (1.15 g, 4.18 mmol). The reaction mixture was stirred at 90° C. overnight. The mixture was diluted with EtOAc, washed with water and brine, dried over anhydrous Na₂SO₄, filtered, and concentrated under reduced pressure to dryness. The residue was purified by flash chromatography (silica gel, 0-66% EtOAc in PE) to give the title compound (70 mg, yield 10.0%) as a white solid. LC/MS (ESI) (m/z): 251 (M+H)⁺.

### Step 2: Thieno[3, 2-c]pyridin-2-amine hydrochloride (3)

To a solution of tert-butyl thieno[3,2-c]pyridin-2-ylcarbamate (70 mg, 0.06 mmol) in DCM (2 mL) was added HCl/1,4-dioxane (1 mL) and the mixture was stirred under N₂ atmosphere at room temperature for 3 hours. The reaction mixture was concentrated under reduced pressure to dryness to give the title compound (50 mg, yield 96.0%) as a yellow solid. LC/MS (ESI) (m/z): 151 (M+H)⁺.

### Step 3: 2-Fluoro-3-(p-tolyloxy)benzonitrile (4)

To a solution of 1-bromo-2-fluoro-3-(p-tolyloxy)benzene (1.30 g, 4.64 mmol) in NMP (13 mL) was added CuCN (1.03 g, 11.60 mmol). The reaction mixture was stirred at 180° C. for 20 minutes in microwave. The mixture was diluted with EtOAc, washed with brine, dried over anhydrous Na₂SO₄, filtered, and concentrated under reduced pressure to dryness. The residue was purified by flash chromatography (silica gel, 0-9% EtOAc in PE) to give the title compound (600 mg, yield 57.1%) as a colorless oil. ¹H NMR (400 MHz, CDCl₃): δ 7.32 (t, J=6.3 Hz, 1H), 7.21 (s. 1H), 7.16 (s, 2H), 7.13 (d, J=8.1 Hz, 1H), 6.91 (d, J=8.1 Hz, 2H), 2.35 (s, 3H).

### Step 4: 2-Fluoro-3-(p-tolyloxy)benzoic acid (5)

To a solution of 2-fluoro-3-(p-tolyloxy)benzonitrile (600 mg, 2.64 mmol) in water (6 mL) was added NaOH (317 mg, 7.92 mmol) and the mixture was stirred at 100° C. overnight. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (580 mg, yield 89.2%) as a white solid, which was used directly in the next step without further purification. LC/MS (ESI) m/z: 247 (M+H)⁺.

### Step 5: Methyl (2-fluoro-3-(p-tolyloxy)benzoyl)glycinate (6)

To a mixture of 2-fluoro-3-(p-tolyloxy)benzoic acid (400 mg, 1.63 mmol) and methyl glycinate hydrochloride (408 mg, 3.25 mmol) in DMF (4 mL) was added DIPEA (1.26 g, 9.76 mmol) and HATU (741 mg, 1.96 mmol). The reaction mixture was stirred at 25° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-23% EtOAc in PE) to give the title compound (400 mg, yield 77.7%) as a colorless oil. LC/MS (ESI) (m/z): 318 (M+H)⁺.

### Step 6: (2-Fluoro-3-(p-tolyloxy)benzoyl)glycine (7)

To a solution of methyl (2-fluoro-3-(p-tolyloxy)benzoyl)glycinate (400 mg, 1.26 mmol) in MeOH (3 mL) and water (1 mL) was added LiOH (91 mg, 3.78 mmol). The reaction mixture was stirred at room temperature for 2 hours. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (360 mg, yield 94.2%) as a yellow solid, which was used directly in the next step without further purification. LC/MS (ESI) m/z: 304 (M+H)⁺.

### Step 7: Methyl (2S,4R)-4-(difluoromethoxy)-1-((2-fluoro-3-(p-tolyloxy)benzoyl) glycyl)pyrrolidine-2-carboxylate (8)

To a mixture of (2-fluoro-3-(p-tolyloxy)benzoyl)glycine (120 mg, 0.39 mmol) and methyl (2S,4R)-4-(difluoromethoxy)pyrrolidine-2-carboxylate hydrochloride (138 mg, 0.58 mmol) in DMF (3 mL) was added DIPEA (306 mg, 2.34 mmol) and HATU (180 mg, 0.47 mmol). The reaction mixture was stirred at 25° C. for 2 hours. The mixture was diluted with EtOAc, washed with water and brine, dried over anhydrous Na₂SO₄, filtered, and concentrated under reduced pressure to dryness. The residue was purified by flash chromatography (silica gel, 0-50% EtOAc in PE) to give the title compound (170 mg, yield 89.5%) as a yellow solid. LC/MS (ESI) (m/z): 481 (M+H)⁺.

### Step 8: (2S,4R)-4-(difluoromethoxy)-1-((2-fluoro-3-(p-tolyloxy)benzoyl)glycyl) pyrrolidine-2-carboxylic acid (9)

To a solution of methyl (2S,4R)-4-(difluoromethoxy)-1-((2-fluoro-3-(p-tolyloxy)benzoyl) glycyl)pyrrolidine-2-carboxylate (70 mg, 0.15 mmol) in MeOH (3 mL) and water (1 mL) was added LiOH (11 mg, 0.45 mmol). The reaction mixture was stirred at room temperature for 2 hours. The mixture was acidified with 1N aq·HCl to pH˜3 and extracted with EtOAc twice. The combined organic layers were washed with brine, dried over anhydrous Na₂SO₄, filtered, and concentrated under reduced pressure to dryness to give the title compound (60 mg, yield 88.4%) as a yellow solid, which was used directly in the next step without further purification. LC/MS (ESI) m/z: 467 (M+H)⁺.

### Step 9: (2S,4R)-4-(difluoromethoxy)-1-((2-fluoro-3-(p-tolyloxy)benzoyl)glycyl)-N-(thieno[3, 2-c]pyridin-2-yl)pyrrolidine-2-carboxamide (Compound 376)

To a mixture of (2S,4R)-4-(difluoromethoxy)-1-((2-fluoro-3-(p-tolyloxy)benzoyl) glycyl)pyrrolidine-2-carboxylic acid (50 mg, 0.11 mmol) and thieno[3,2-c]pyridin-2-amine hydrochloride (30 mg, 0.16 mmol) in DMF (3 mL) was added DIPEA (53 mg, 0.66 mmol) and HATU (90 mg, 0.16 mmol) at 0° C. and the mixture was stirred at room temperature for 2 hours. The mixture was extracted with EtOAc twice, washed with brine, dried over anhydrous Na₂SO₄, filtered, and concentrated to dryness under reduced pressure. The residue was purified by prep-TLC (DCM:MeOH=13:1) and further purified by prep-HPLC to give Compound 376 (10 mg, yield 15.6%) as a white solid. ¹H NMR (400 MHz, CD₃OD): δ 8.85 (s, 1H), 8.24 (s, 1H), 7.87 (d, J=5.4 Hz, 1H), 7.54 (d, J=6.4 Hz, 1H), 7.19-7.10 (m. 5H), 6.85 (d, J=7.7 Hz, 2H), 6.54 (t, J=74.3 Hz, 1H), 5.08 (s, 1H), 4.74 (t, J=7.7 Hz, 1H), 4.37 (d, J=17.1 Hz, 1H₁), 4.23 (d, J=17.0 Hz, 1H), 4.04-3.99 (m, 1H), 3.91 (d, J=11.9 Hz, 1H), 2.58-2.50 (in, 1H), 2.35-2.30 (in, 1H), 2.29 (s, 3H₁). LC/MS (ESI) (m/z): 599 (M+H). RT (Method A): 1.68 min.

### Example 2. NON-LIMITING EXAMPLES OF COMPOUNDS OF THE PRESENT DISCLOSURE

Table 1 shows illustrative complement pathway inhibitors.

TABLE 1
Non-limiting Examples of Compounds of the Present Disclosure

| # | Structure | Name |
|---|---|---|
| 1 | 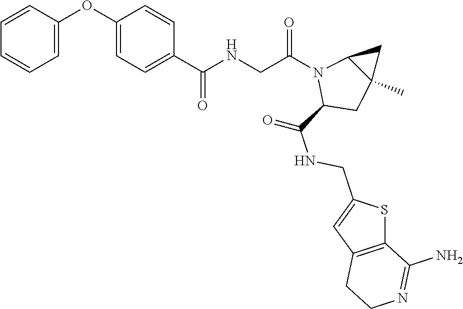 [SMILES: C[C@@]12C[C@@H]1N(C(=O)CNC(=O)c1ccc(Oc3ccccc3)cc1)[C@H](C(=O)NCc1cc3c(s1)C(N)=NCC3)C2] | (1S,3S,5S)-N-((7-amino-4,5- dihydrothieno[2,3-c]pyridin-2- yl)methyl)-5-methyl-2-((4- phenoxybenzoyl)glycyl)-2- azabicyclo[3.1.0]hexane-3- carboxamide |
| 2 | 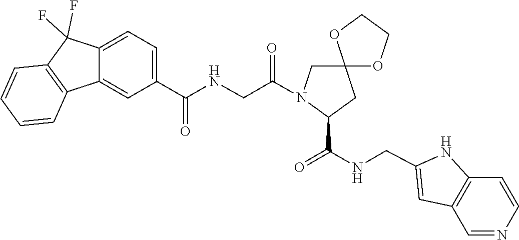 [SMILES: O=C(NCC(=O)N1CC2(C[C@H]1C(=O)NCc1cc3cnccc3[nH]1)OCCO2)c1ccc2c(c1)-c1ccccc1C2(F)F] | (S)-N-((1H-pyrrolo[3,2-c]pyridin- 2-yl)methyl)-7-((9,9-difluoro-9H- fluorene-3-carbonyl)glycyl)-1,4- dioxa-7-azaspiro[4.4]nonane-8- carboxamide |
| 3 | 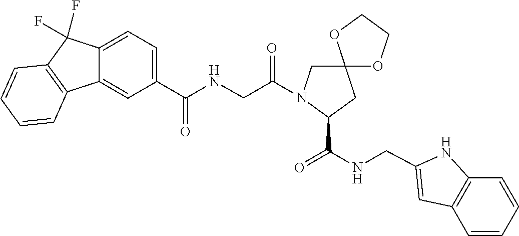 [SMILES: O=C(NCC(=O)N1CC2(C[C@H]1C(=O)NCc1cc3ccccc3[nH]1)OCCO2)c1ccc2c(c1)-c1ccccc1C2(F)F] | (S)-N-((1H-indol-2-yl)methyl)-7- ((9,9-difluoro-9H-fluorene-3- carbonyl)glycyl)-1,4-dioxa-7- azaspiro[4.4]nonane-8- carboxamide |
| 4 | 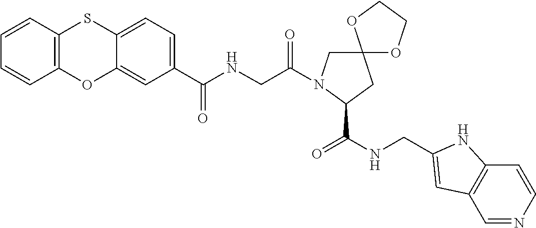 [SMILES: O=C(NCC(=O)N1CC2(C[C@H]1C(=O)NCc1cc3cnccc3[nH]1)OCCO2)c1ccc2c(c1)Oc1ccccc1S2] | (S)-N-((1H-pyrrolo[3,2-c]pyridin- 2-yl)methyl)-7-((phenoxathiine-3- carbonyl)glycyl)-1,4-dioxa-7- azaspiro[4.4]nonane-8- carboxamide |
| 5 | 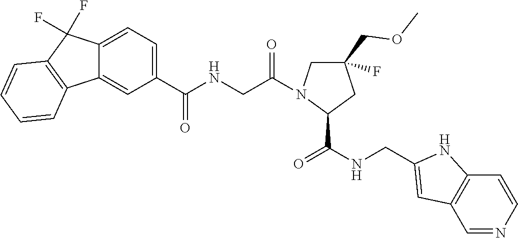 [SMILES: COC[C@@]1(F)C[C@@H](C(=O)NCc2cc3cnccc3[nH]2)N(C(=O)CNC(=O)c2ccc3c(c2)-c2ccccc2C3(F)F)C1] | (2S,4R)-N-((1H-pyrrolo[3,2- c]pyridin-2-yl)methyl)-1-((9,9- difluoro-9H-fluorene-3- carbonyl)glycyl)-4-fluoro-4- (methoxymethyl)pyrrolidine-2- carboxamide |
| 6 | 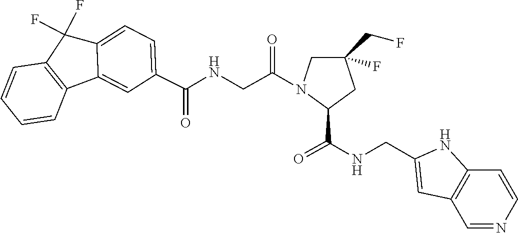 [SMILES: O=C(NCC(=O)N1C[C@@](F)(CF)C[C@H]1C(=O)NCc1cc2cnccc2[nH]1)c1ccc2c(c1)-c1ccccc1C2(F)F] | (2S,4R)-N-((1H-pyrrolo[3,2- c]pyridin-2-yl)methyl)-1-((9,9- difluoro-9H-fluorene-3- carbonyl)glycyl)-4-fluoro-4- (fluoromethyl)pyrrolidine-2- carboxamide |
| 7 | 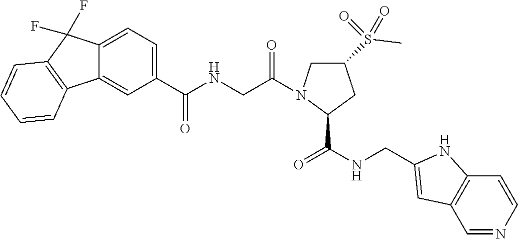 [SMILES: CS(=O)(=O)[C@@H]1C[C@@H](C(=O)NCc2cc3cnccc3[nH]2)N(C(=O)CNC(=O)c2ccc3c(c2)-c2ccccc2C3(F)F)C1] | (2S,4R)-N-((1H-pyrrolo[3,2- c]pyridin-2-yl)methyl)-1-((9,9- difluoro-9H-fluorene-3- carbonyl)glycyl)-4- (methylsulfonyl)pyrrolidine-2- carboxamide |
| 8 | 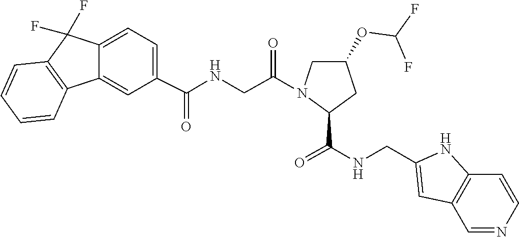 [SMILES: O=C(NCC(=O)N1C[C@H](OC(F)F)C[C@H]1C(=O)NCc1cc2cnccc2[nH]1)c1ccc2c(c1)-c1ccccc1C2(F)F] | (2S,4R)-N-((1H-pyrrolo[3,2- c]pyridin-2-yl)methyl)-1-((9,9- difluoro-9H-fluorene-3- carbonyl)glycyl)-4- (difluoromethoxy)pyrrolidine-2- carboxamide |
| 9 | 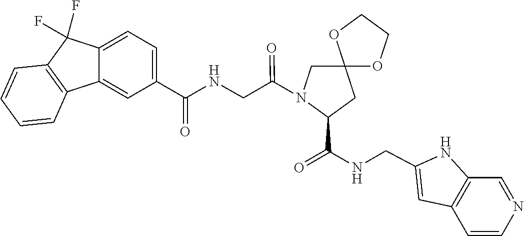 [SMILES: O=C(NCC(=O)N1CC2(C[C@H]1C(=O)NCc1cc3ccncc3[nH]1)OCCO2)c1ccc2c(c1)-c1ccccc1C2(F)F] | (S)-N-((1H-pyrrolo[2,3-c]pyridin- 2-yl)methyl)-7-((9,9-difluoro-9H- fluorene-3-carbonyl)glycyl)-1,4- dioxa-7-azaspiro[4.4]nonane-8- carboxamide |
| 10 | 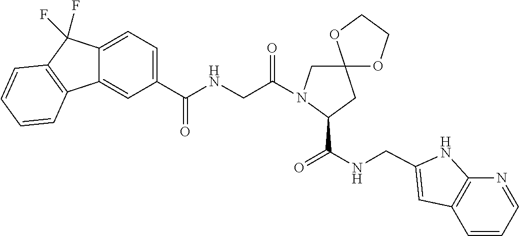 [SMILES: O=C(NCC(=O)N1CC2(C[C@H]1C(=O)NCc1cc3cccnc3[nH]1)OCCO2)c1ccc2c(c1)-c1ccccc1C2(F)F] | (S)-N-((1H-pyrrolo[2,3-b]pyridin- 2-yl)methyl)-7-((9,9-difluoro-9H- fluorene-3-carbonyl)glycyl)-1,4- dioxa-7-azaspiro[4.4]nonane-8- carboxamide |
| 11 | 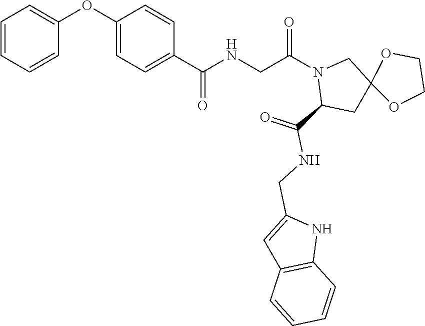 [SMILES: O=C(NCC(=O)N1CC2(C[C@H]1C(=O)NCc1cc3ccccc3[nH]1)OCCO2)c1ccc(Oc2ccccc2)cc1] | (S)-N-((1H-indol-2-yl)methyl)-7- ((4-phenoxybenzoyl)glycyl)-1,4- dioxa-7-azaspiro[4.4]nonane-8- carboxamide |
| 12 | 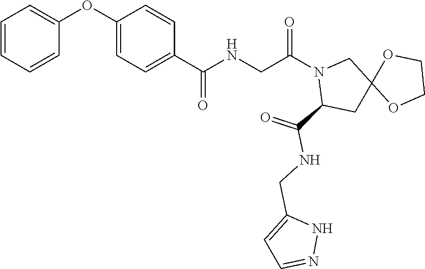 [SMILES: O=C(NCC(=O)N1CC2(C[C@H]1C(=O)NCc1ccn[nH]1)OCCO2)c1ccc(Oc2ccccc2)cc1] | (S)-N-((1H-pyrazol-5-yl)methyl)- 7-((4-phenoxybenzoyl)glycyl)- 1,4-dioxa-7-azaspiro[4.4]nonane-8- carboxamide |
| 13 | 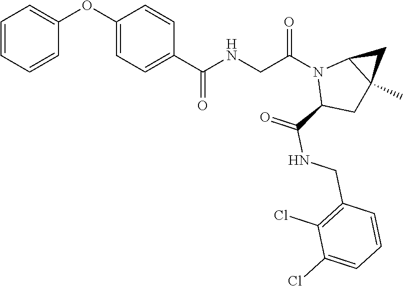 [SMILES: C[C@@]12C[C@@H]1N(C(=O)CNC(=O)c1ccc(Oc3ccccc3)cc1)[C@H](C(=O)NCc1cccc(Cl)c1Cl)C2] | (1S,3S,5S)-N-(2,3- dichlorobenzyl)-5-methyl-2-((4- phenoxybenzoyl)glycyl)-2- azabicyclo[3.1.0]hexane-3- carboxamide |
| 14 | 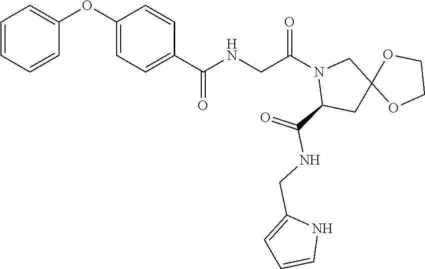 [SMILES: O=C(NCC(=O)N1CC2(C[C@H]1C(=O)NCc1ccc[nH]1)OCCO2)c1ccc(Oc2ccccc2)cc1] | (S)-N-((1H-pyrrol-2-yl)methyl)-7- ((4-phenoxybenzoyl)glycyl)-1,4- dioxa-7-azaspiro[4.4]nonane-8- carboxamide |
| 15 | 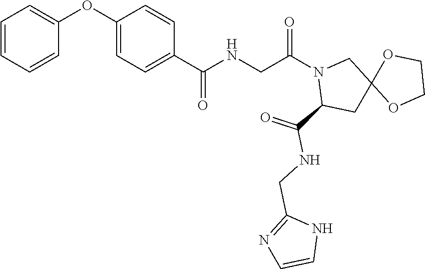 [SMILES: O=C(NCC(=O)N1CC2(C[C@H]1C(=O)NCc1ncc[nH]1)OCCO2)c1ccc(Oc2ccccc2)cc1] | (S)-N-((1H-imidazol-2- yl)methyl)-7-((4- phenoxybenzoyl)glycyl)-1,4- dioxa-7-azaspiro[4.4]nonane-8- carboxamide |
| 16 | 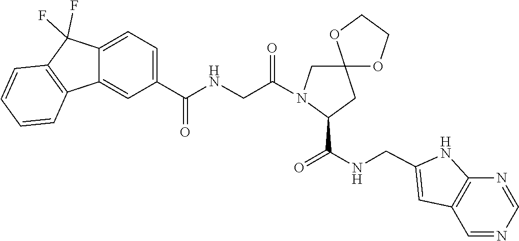 [SMILES: O=C(NCC(=O)N1CC2(C[C@H]1C(=O)NCc1cc3cncnc3[nH]1)OCCO2)c1ccc2c(c1)-c1ccccc1C2(F)F] | (S)-N-((7H-pyrrolo[2,3- d]pyrimidin-6-yl)methyl)-7-((9,9- difluoro-9H-fluorene-3- carbonyl)glycyl)-1,4-dioxa-7- azaspiro[4.4]nonane-8- carboxamide |
| 17 | 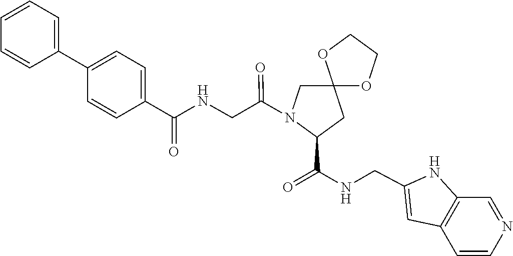 [SMILES: O=C(NCC(=O)N1CC2(C[C@H]1C(=O)NCc1cc3ccncc3[nH]1)OCCO2)c1ccc(-c2ccccc2)cc1] | (S)-N-((1H-pyrrolo[3,2-c]pyridin- 2-yl)methyl)-7-(([1,1′-biphenyl]- 4-carbonyl)glycyl)-1,4-dioxa-7- azaspiro[4.4]nonane-8- carboxamide |
| 18 | 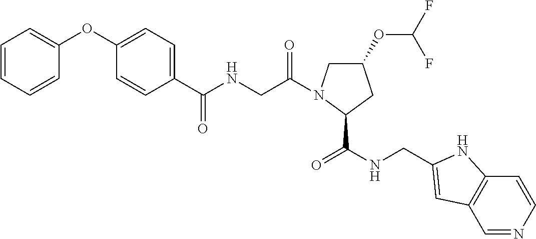 [SMILES: O=C(NCC(=O)N1C[C@H](OC(F)F)C[C@H]1C(=O)NCc1cc2cnccc2[nH]1)c1ccc(Oc2ccccc2)cc1] | (2S,4R)-N-((1H-pyrrolo[3,2- c]pyridin-2-yl)methyl)-4- (difluoromethoxy)-1-((4- phenoxybenzoyl)glycyl) pyrrolidine-2-carboxamide |
| 19 | 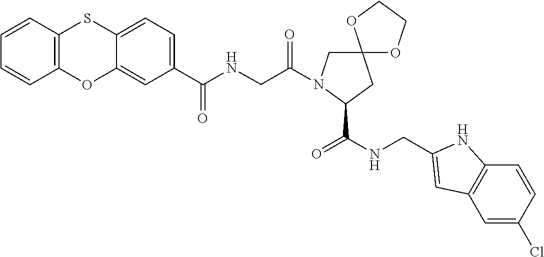 [SMILES: O=C(NCC(=O)N1CC2(C[C@H]1C(=O)NCc1cc3cc(Cl)ccc3[nH]1)OCCO2)c1ccc2c(c1)Oc1ccccc1S2] | (S)-N-((5-chloro-1H-indol-2- yl)methyl)-7-((phenoxathiine-3- carbonyl)glycyl)-1,4-dioxa-7- azaspiro[4.4]nonane-8- carboxamide |
| 20 | 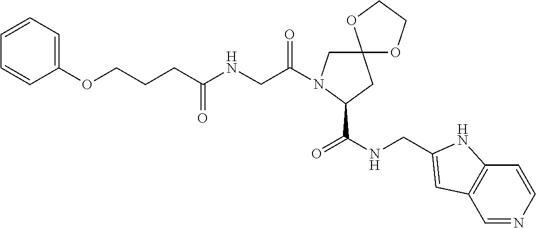 [SMILES: O=C(CCCOc1ccccc1)NCC(=O)N1CC2(C[C@H]1C(=O)NCc1cc3cnccc3[nH]1)OCCO2] | (S)-N-((1H-pyrrolo[3,2-c]pyridin- 2-yl)methyl)-7-((4- phenoxybutanoyl)glycyl)-1,4- dioxa-7-azaspiro[4.4]nonane-8- carboxamide |
| 21 | 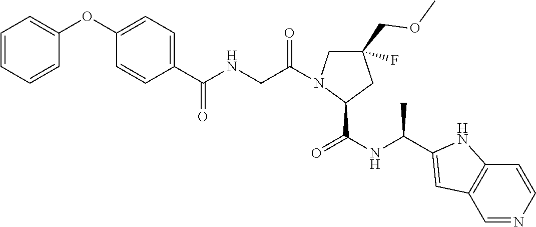 [SMILES: COC[C@@]1(F)C[C@@H](C(=O)N[C@@H](C)c2cc3cnccc3[nH]2)N(C(=O)CNC(=O)c2ccc(Oc3ccccc3)cc2)C1] | (2S,4R)-N-((S)-1-(1H- pyrrolo[3,2-c]pyridin-2-yl)ethyl)- 4-fluoro-4-(methoxymethyl)-1- ((4- phenoxybenzoyl)glycyl) pyrrolidine-2-carboxamide |
| 22 | 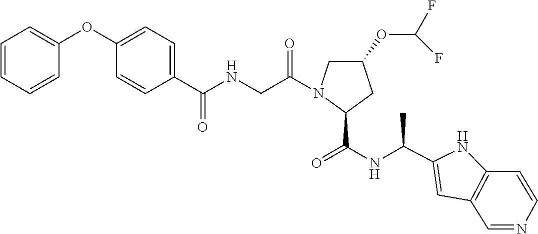 [SMILES: C[C@H](NC(=O)[C@@H]1C[C@@H](OC(F)F)CN1C(=O)CNC(=O)c1ccc(Oc2ccccc2)cc1)c1cc2cnccc2[nH]1] | (2S,4R)-N-((S)-1-(1H- pyrrolo[3,2-c]pyridin-2-yl)ethyl)- 4-(difluoromethoxy)-1-((4- phenoxybenzoyl)glycyl) pyrrolidine-2-carboxamide |
| 23 | 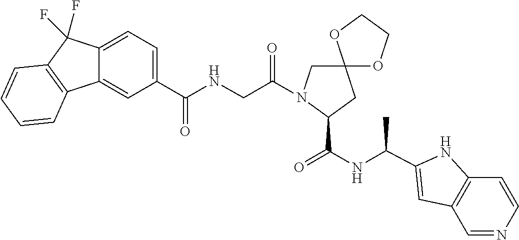 [SMILES: C[C@H](NC(=O)[C@@H]1CC2(CN1C(=O)CNC(=O)c1ccc3c(c1)-c1ccccc1C3(F)F)OCCO2)c1cc2cnccc2[nH]1] | (S)-N-((S)-1-(1H-pyrrolo[3,2- c]pyridin-2-yl)ethyl)-7-((9,9- difluoro-9H-fluorene-3- carbonyl)glycyl)-1,4-dioxa-7- azaspiro[4.4]nonane-8- carboxamide |
| 24 | 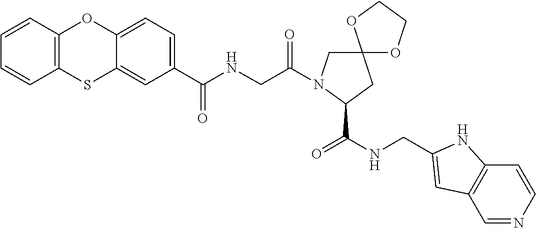 [SMILES: O=C(NCC(=O)N1CC2(C[C@H]1C(=O)NCc1cc3cnccc3[nH]1)OCCO2)c1ccc2c(c1)Sc1ccccc1O2] | (S)-N-((1H-pyrrolo[3,2-c]pyridin- 2-yl)methyl)-7-((phenoxathiine-2- carbonyl)glycyl)-1,4-dioxa-7- azaspiro[4.4]nonane-8- carboxamide |
| 25 | 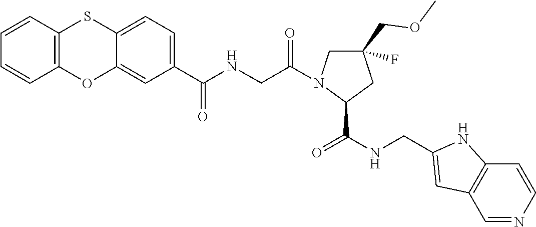 [SMILES: COC[C@@]1(F)C[C@@H](C(=O)NCc2cc3cnccc3[nH]2)N(C(=O)CNC(=O)c2ccc3c(c2)Oc2ccccc2S3)C1] | (2S,4R)-N-((1H-pyrrolo[3,2- c]pyridin-2-yl)methyl)-4-fluoro-4- (methoxymethyl)-1- ((phenoxathiine-3- carbonyl)glycyl)pyrrolidine-2- carboxamide |
| 26 | 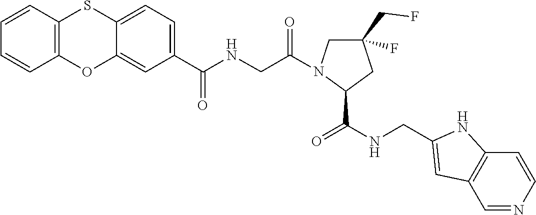 [SMILES: O=C(NCC(=O)N1C[C@@](F)(CF)C[C@H]1C(=O)NCc1cc2cnccc2[nH]1)c1ccc2c(c1)Oc1ccccc1S2] | (2S,4R)-N-((1H-pyrrolo[3,2- c]pyridin-2-yl)methyl)-4-fluoro-4- (fluoromethyl)-1-((phenoxathiine- 3-carbonyl)glycyl)pyrrolidine-2- carboxamide |
| 27 | 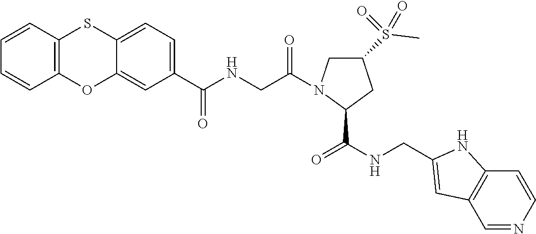 [SMILES: CS(=O)(=O)[C@@H]1C[C@@H](C(=O)NCc2cc3cnccc3[nH]2)N(C(=O)CNC(=O)c2ccc3c(c2)Oc2ccccc2S3)C1] | (2S,4R)-N-((1H-pyrrolo[3,2- c]pyridin-2-yl)methyl)-4- (methylsulfonyl)-1- ((phenoxathiine-3- carbonyl)glycyl)pyrrolidine-2- carboxamide |
| 28 | 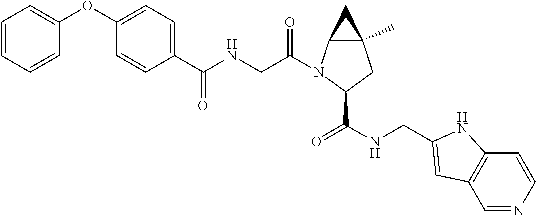 [SMILES: C[C@@]12C[C@@H]1N(C(=O)CNC(=O)c1ccc(Oc3ccccc3)cc1)[C@H](C(=O)NCc1cc3cnccc3[nH]1)C2] | (1S,3S,5S)-N-((1H-pyrrolo[3,2- c]pyridin-2-yl)methyl)-5-methyl- 2-((4-phenoxybenzoyl)glycyl)-2- azabicyclo[3.1.0]hexane-3- carboxamide |
| 29 | 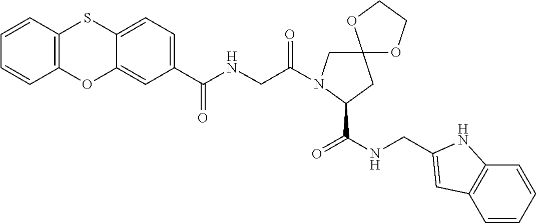 [SMILES: O=C(NCC(=O)N1CC2(C[C@H]1C(=O)NCc1cc3ccccc3[nH]1)OCCO2)c1ccc2c(c1)Oc1ccccc1S2] | (S)-N-((1H-indol-2-yl)methyl)-7- ((phenoxathiine-3- carbonyl)glycyl)-1,4-dioxa-7- azaspiro[4.4]nonane-8- carboxamide |
| 30 | 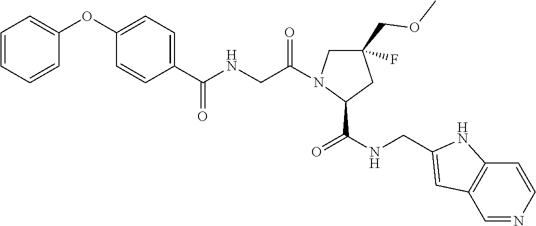 [SMILES: COC[C@@]1(F)C[C@@H](C(=O)NCc2cc3cnccc3[nH]2)N(C(=O)CNC(=O)c2ccc(Oc3ccccc3)cc2)C1] | (2S,4R)-N-((1H-pyrrolo[3,2- c]pyridin-2-yl)methyl)-4-fluoro-4- (methoxymethyl)-1-((4- phenoxybenzoyl)glycyl) pyrrolidine-2-carboxamide |
| 31 | 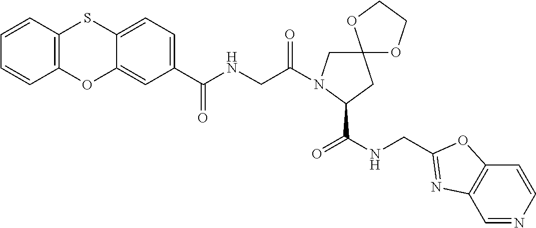 [SMILES: O=C(NCC(=O)N1CC2(C[C@H]1C(=O)NCc1nc3cnccc3o1)OCCO2)c1ccc2c(c1)Oc1ccccc1S2] | (S)-N-(oxazolo[4,5-c]pyridin-2- ylmethyl)-7-((phenoxathiine-3- carbonyl)glycyl)-1,4-dioxa-7- azaspiro[4.4]nonane-8- carboxamide |
| 32 | 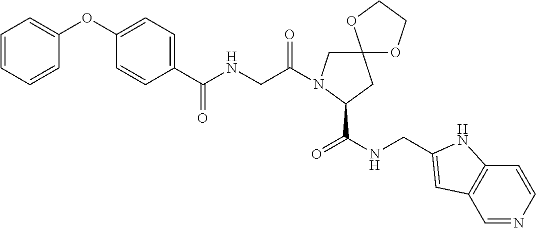 [SMILES: O=C(NCC(=O)N1CC2(C[C@H]1C(=O)NCc1cc3cnccc3[nH]1)OCCO2)c1ccc(Oc2ccccc2)cc1] | (S)-N-((1H-pyrrolo[3,2-]pyridin- 2-yl)methyl)-7-((4- phenoxybenzoyl)glycyl)-1,4- dioxa-7-azaspiro[4.4]nonane-8- carboxamide |
| 33 | 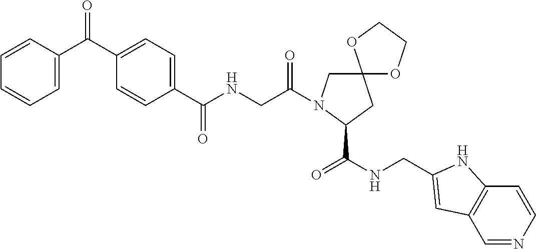 [SMILES: O=C(NCC(=O)N1CC2(C[C@H]1C(=O)NCc1cc3cnccc3[nH]1)OCCO2)c1ccc(C(=O)c2ccccc2)cc1] | (S)-N-((1H-pyrrolo[3,2-c]pyridin- 2-yl)methyl)-7-((4- benzoylbenzoyl)glycyl)-1,4- dioxa-7-azaspiro[4.4]nonane-8- carboxamide |
| 34 | 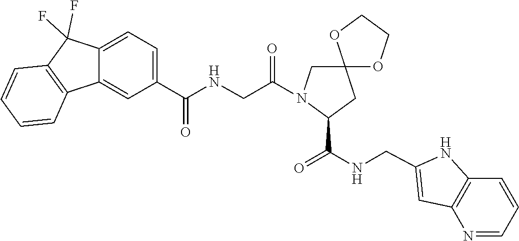 [SMILES: O=C(NCC(=O)N1CC2(C[C@H]1C(=O)NCc1cc3ncccc3[nH]1)OCCO2)c1ccc2c(c1)-c1ccccc1C2(F)F] | (S)-N-((1H-pyrrolo[3,2-c]pyridin- 2-yl)methyl)-7-((9,9-difluoro-9H- fluorene-3-carbonyl)glycyl)-1,4- dioxa-7-azaspiro[4.4]nonane-8- carboxamide |
| 35 | 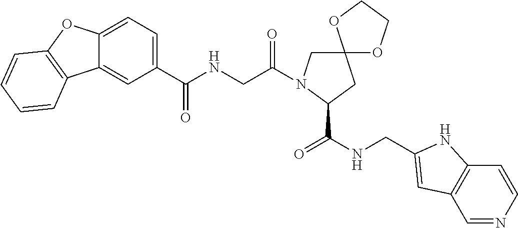 [SMILES: O=C(NCC(=O)N1CC2(C[C@H]1C(=O)NCc1cc3cnccc3[nH]1)OCCO2)c1ccc2oc3ccccc3c2c1] | (S)-N-((1H-pyrrolo[3,2-c]pyridin- 2-yl)methyl)-7- ((dibenzo[b,d]furan-2- carbonyl)glycyl)-1,4-dioxa-7- azaspiro[4.4]nonane-8- carboxamide |
| 36 | 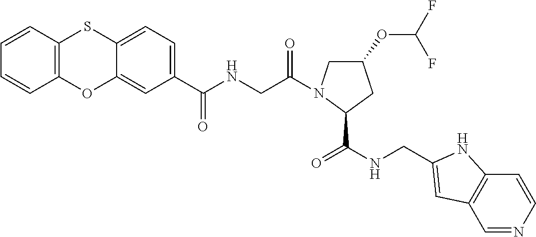 [SMILES: O=C(NCC(=O)N1C[C@H](OC(F)F)C[C@H]1C(=O)NCc1cc2cnccc2[nH]1)c1ccc2c(c1)Oc1ccccc1S2] | (2S,4R)-N-((1H-pyrrolo[3,2- c]pyridin-2-yl)methyl)-4- (difluoromethoxy)-1- ((phenoxathiine-3- carbonyl)glycyl)pyrrolidine-2- carboxamide |
| 37 | 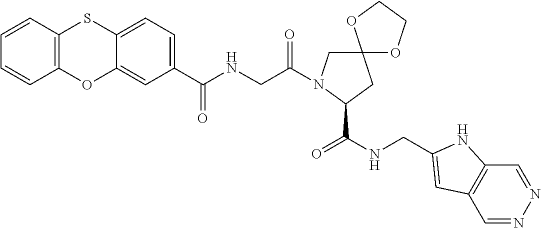 [SMILES: O=C(NCC(=O)N1CC2(C[C@H]1C(=O)NCc1cc3cnncc3[nH]1)OCCO2)c1ccc2c(c1)Oc1ccccc1S2] | (S)-N-((1H-pyrrolo[2,3- d]pyridazin-2-yl)methyl)-7- ((phenoxathiine-3- carbonyl)glycyl)-1,4-dioxa-7- azaspiro[4.4]nonane-8- carboxamide |
| 38 | 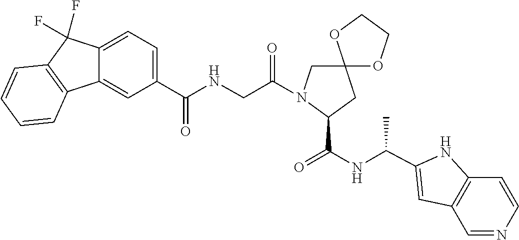 [SMILES: C[C@@H](NC(=O)[C@@H]1CC2(CN1C(=O)CNC(=O)c1ccc3c(c1)-c1ccccc1C3(F)F)OCCO2)c1cc2cnccc2[nH]1] | (S)-N-((R)-1-(1H-pyrrolo[3,2- c]pyridin-2-yl)ethyl)-7-((9,9- difluoro-9H-fluorene-3- carbonyl)glycyl)-1,4-dioxa-7- azaspiro[4.4]nonane-8- carboxamide |
| 39 | 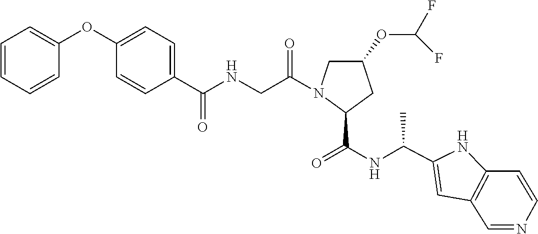 [SMILES: C[C@@H](NC(=O)[C@@H]1C[C@@H](OC(F)F)CN1C(=O)CNC(=O)c1ccc(Oc2ccccc2)cc1)c1cc2cnccc2[nH]1] | (2S,4R)-N-((R)-1-(1H- pyrrolo[3,2-c]pyridin-2-yl)ethyl)- 4-(difluoromethoxy)-1-((4- phenoxybenzoyl)glycyl) pyrrolidine-2-carboxamide |
| 40 | 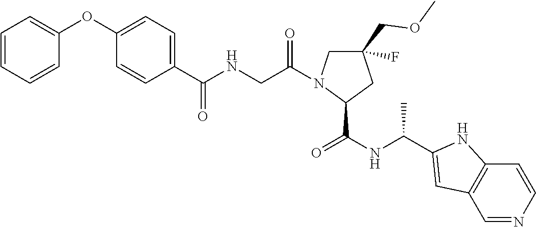 [SMILES: COC[C@@]1(F)C[C@@H](C(=O)N[C@H](C)c2cc3cnccc3[nH]2)N(C(=O)CNC(=O)c2ccc(Oc3ccccc3)cc2)C1] | (2S,4R)-N-((R)-1-(1H- pyrrolo[3,2-c]pyridin-2-yl)ethyl)- 4-fluoro-4-(methoxymethyl)-1- ((4- phenoxybenzoyl)glycyl) pyrrolidine-2-carboxamide |
| 41 | 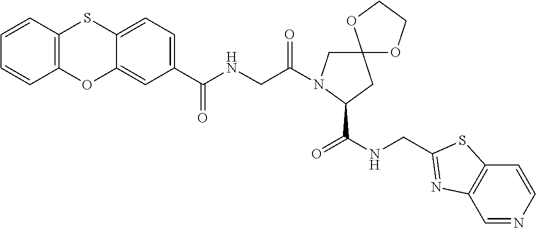 [SMILES: O=C(NCC(=O)N1CC2(C[C@H]1C(=O)NCc1nc3cnccc3s1)OCCO2)c1ccc2c(c1)Oc1ccccc1S2] | (S)-7-((phenoxathine-3- carbonyl)glycyl)-N-(thiazolo[4,5- c]pyridin-2-ylmethyl)-1,4-dioxa- 7-azaspiro[4.4]nonane-8- carboxamide |
| 42 | 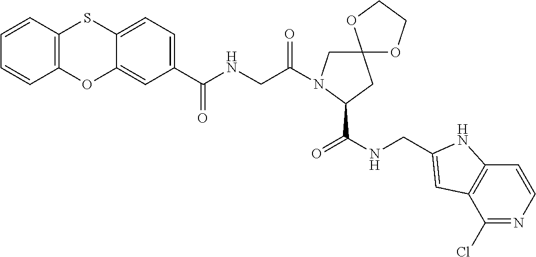 [SMILES: O=C(NCC(=O)N1CC2(C[C@H]1C(=O)NCc1cc3c(Cl)nccc3[nH]1)OCCO2)c1ccc2c(c1)Oc1ccccc1S2] | (S)-N-((4-chloro-1H-pyrrolo[3,2- c]pyridin-2-yl)methyl)-7- ((phenoxathiine-3- carbonyl)glycyl)-1,4-dioxa-7- azaspiro[4.4]nonane-8- carboxamide |
| 43 | 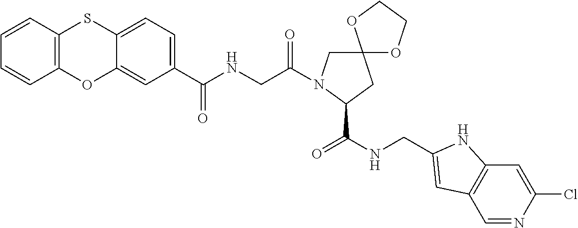 [SMILES: O=C(NCC(=O)N1CC2(C[C@H]1C(=O)NCc1cc3cnc(Cl)cc3[nH]1)OCCO2)c1ccc2c(c1)Oc1ccccc1S2] | (S)-N-((6-chloro-1H-pyrrolo[3,2- c]pyridin-2-yl)methyl)-7- ((phenoxathiine-3- carbonyl)glycyl)-1,4-dioxa-7- azaspiro[4.4]nonane-8- carboxamide |
| 44 | 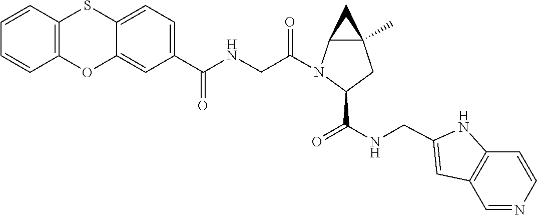 [SMILES: C[C@@]12C[C@@H]1N(C(=O)CNC(=O)c1ccc3c(c1)Oc1ccccc1S3)[C@H](C(=O)NCc1cc3cnccc3[nH]1)C2] | (1S,3S,5S)-N-((1H-pyrrolo[3,2- c]pyridin-2-yl)methyl)-5-methyl- 2-((phenoxathiine-3- carbonyl)glycyl)-2- azabicyclo[3.1.0]hexane-3- carboxamide |
| 45 | 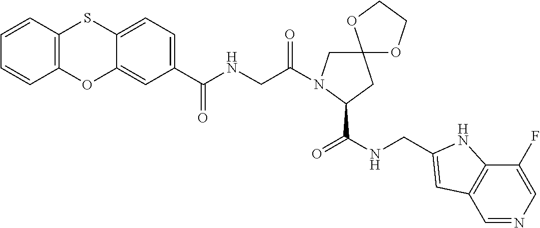 [SMILES: O=C(NCC(=O)N1CC2(C[C@H]1C(=O)NCc1cc3cncc(F)c3[nH]1)OCCO2)c1ccc2c(c1)Oc1ccccc1S2] | (S)-N-((7-fluoro-1H-pyrrolo[3,2- c]pyridin-2-yl)methyl)-7- ((phenoxathiine-3- carbonyl)glycyl)-1,4-dioxa-7- azaspiro[4.4]nonane-8- carboxamide |
| 46 | 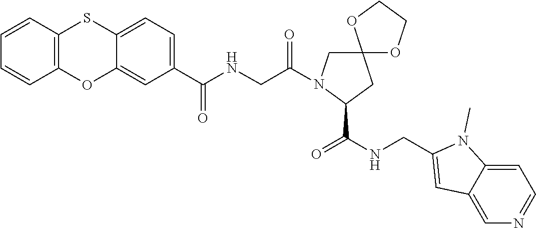 [SMILES: Cn1c(CNC(=O)[C@@H]2CC3(CN2C(=O)CNC(=O)c2ccc4c(c2)Oc2ccccc2S4)OCCO3)cc2cnccc21] | (S)-N-((1-methyl-1H-pyrrolo[3,2- c]pyridin-2-yl)methyl)-7- ((phenoxathiine-3- carbonyl)glycyl)-1,4-dioxa-7- azaspiro[4.4]nonane-8- carboxamide |
| 47 | 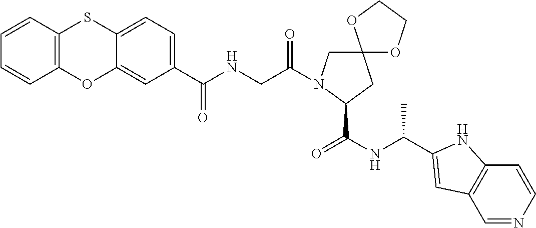 [SMILES: C[C@@H](NC(=O)[C@@H]1CC2(CN1C(=O)CNC(=O)c1ccc3c(c1)Oc1ccccc1S3)OCCO2)c1cc2cnccc2[nH]1] | (S)-N-((R)-1-(1H-pyrrolo[3,2- c]pyridin-2-yl)ethyl)-7- ((phenoxathiine-3- carbonyl)glycyl)-1,4-dioxa-7- azaspiro[4.4]nonane-8- carboxamide |
| 48 | 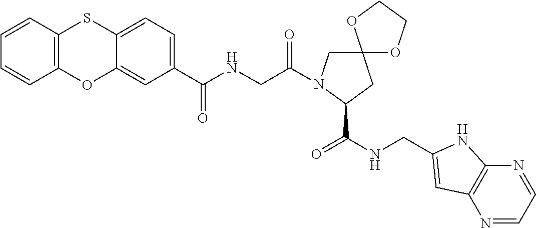 [SMILES: O=C(NCC(=O)N1CC2(C[C@H]1C(=O)NCc1cc3nccnc3[nH]1)OCCO2)c1ccc2c(c1)Oc1ccccc1S2] | (S)-N-((5H-pyrrolo[2,3-b]pyrazin- 6-yl)methyl)-7-((phenoxathiine-3- carbonyl)glycyl)-1,4-dioxa-7- azaspiro[4.4]nonane-8- carboxamide |
| 49 | 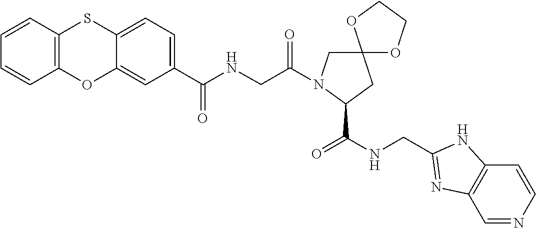 [SMILES: O=C(NCC(=O)N1CC2(C[C@H]1C(=O)NCc1nc3cnccc3[nH]1)OCCO2)c1ccc2c(c1)Oc1ccccc1S2] | (S)-N-((1H-imidazo[4,5- c]pyridin-2-yl)methyl)-7- ((phenoxathiine-3- carbonyl)glycyl)-1,4-dioxa-7- azaspiro[4.4]nonane-8- carboxamide |
| 50 | 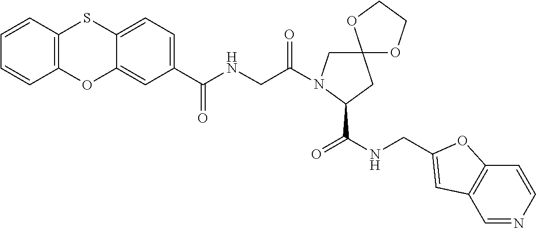 [SMILES: O=C(NCC(=O)N1CC2(C[C@H]1C(=O)NCc1cc3cnccc3o1)OCCO2)c1ccc2c(c1)Oc1ccccc1S2] | (S)-N-(furo[3,2-c]pyridin-2- ylmethyl)-7-((phenoxathiine-3- carbonyl)glycyl)-1,4-dioxa-7- azaspiro[4.4]nonane-8- carboxamide |
| 51 | 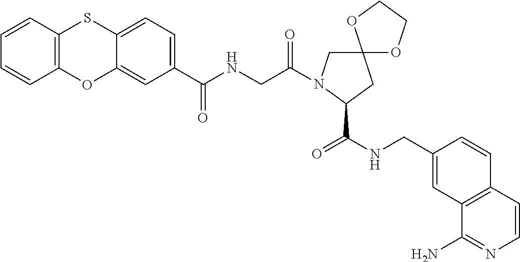 [SMILES: Nc1nccc2ccc(CNC(=O)[C@@H]3CC4(CN3C(=O)CNC(=O)c3ccc5c(c3)Oc3ccccc3S5)OCCO4)cc12] | (S)-N-((1-aminoisoquinolin-7- yl)methyl)-7-((phenoxathiine-3- carbonyl)glycyl)-1,4-dioxa-7- azaspiro[4.4]nonane-8- carboxamide |
| 52 | 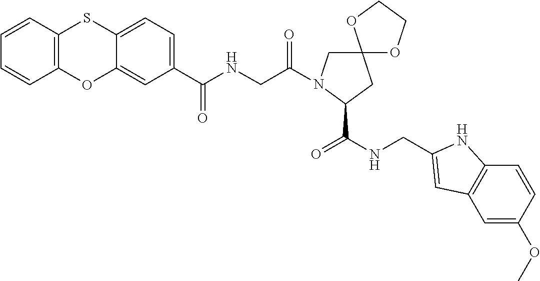 [SMILES: COc1ccc2[nH]c(CNC(=O)[C@@H]3CC4(CN3C(=O)CNC(=O)c3ccc5c(c3)Oc3ccccc3S5)OCCO4)cc2c1] | (S)-N-((5-methoxy-1H-indol-2- yl)methyl)-7-((phenoxathiine-3- carbonyl)glycyl)-1,4-dioxa-7- azaspiro[4.4]nonane-8- carboxamide |
| 53 | 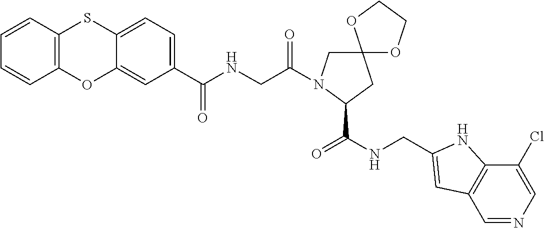 [SMILES: O=C(NCC(=O)N1CC2(C[C@H]1C(=O)NCc1cc3cncc(Cl)c3[nH]1)OCCO2)c1ccc2c(c1)Oc1ccccc1S2] | (S)-N-((7-chloro-1H-pyrrolo[3,2- c]pyridin-2-yl)methyl)-7- ((phenoxathiine-3- carbonyl)glycyl)-1,4-dioxa-7- azaspiro[4.4]nonane-8- carboxamide |
| 54 | 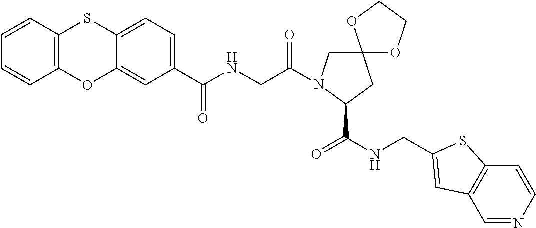 [SMILES: O=C(NCC(=O)N1CC2(C[C@H]1C(=O)NCc1cc3cnccc3s1)OCCO2)c1ccc2c(c1)Oc1ccccc1S2] | (S)-7-((phenoxathiine-3- carbonyl)glycyl)-N-(thieno[3,2- c]pyridin-2-ylmethyl)-1,4-dioxa- 7-azaspiro[4.4]nonane-8- carboxamide |
| 55 | 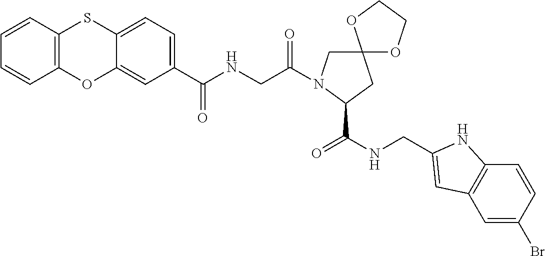 [SMILES: O=C(NCC(=O)N1CC2(C[C@H]1C(=O)NCc1cc3cc(Br)ccc3[nH]1)OCCO2)c1ccc2c(c1)Oc1ccccc1S2] | (S)-N-((5-bromo-1H-indol-2- yl)methyl)-7-((phenoxathiine-3- carbonyl)glycyl)-1,4-dioxa-7- azaspiro[4.4]nonane-8- carboxamide |
| 56 | 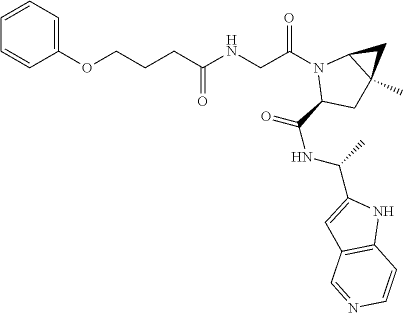 [SMILES: C[C@@H](NC(=O)[C@@H]1C[C@]2(C)C[C@@H]2N1C(=O)CNC(=O)CCCOc1ccccc1)c1cc2cnccc2[nH]1] | (1S,3S,5S)-N-((R)-1-(1H- pyrrolo[3,2-c]pyridin-2-yl)ethyl)- 5-methyl-2-((4- phenoxybutanoyl)glycyl)-2- azabicyclo[3.1.0]hexane-3- carboxamide |
| 57 | 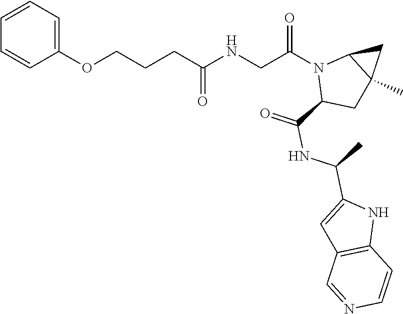 [SMILES: C[C@H](NC(=O)[C@@H]1C[C@]2(C)C[C@@H]2N1C(=O)CNC(=O)CCCOc1ccccc1)c1cc2cnccc2[nH]1] | (1S,3S,5S)-N-((S)-1-(1H- pyrrolo[3,2-c]pyridin-2-yl)ethyl)- 5-methyl-2-((4- phenoxybutanoyl)glycyl)-2- azabicyclo[3.1.0]hexane-3- carboxamide |
| 58 | 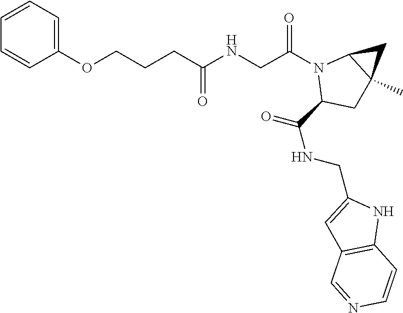 [SMILES: C[C@@]12C[C@@H]1N(C(=O)CNC(=O)CCCOc1ccccc1)[C@H](C(=O)NCc1cc3cnccc3[nH]1)C2] | (1S,3S,5S)-N-((1H-pyrrolo[3,2- c]pyridin-2-yl)methyl)-5-methyl- 2-((4-phenoxybutanoyl)glycyl)-2- azabicyclo[3.1.0]hexane-3- carboxamide |
| 59 | 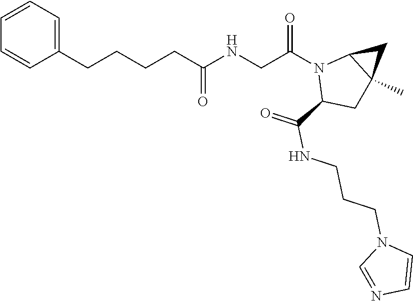 [SMILES: C[C@@]12C[C@@H]1N(C(=O)CNC(=O)CCCCc1ccccc1)[C@H](C(=O)NCCCn1ccnc1)C2] | (1S,3S,5S)-N-(3-(1H-imidazol-1- yl)propyl)-5-methyl-2-((4- phenoxybutanoyl)glycyl)-2- azabicyclo[3.1.0]hexane-3- carboxamide |
| 60 | 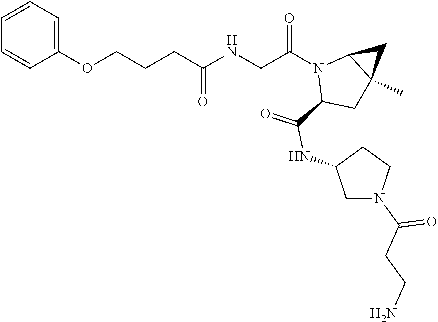 [SMILES: C[C@@]12C[C@@H]1N(C(=O)CNC(=O)CCCOc1ccccc1)[C@H](C(=O)N[C@@H]1CCN(C(=O)CCN)C1)C2] | (1S,3S,5S)-N-((R)-1-(3- aminopropanoyl)pyrrolidin-3-yl)- 5-methyl-2-((4- phenoxybutanoyl)glycyl)-2- azabicyclo[3.1.0]hexane-3- carboxamide |
| 61 | 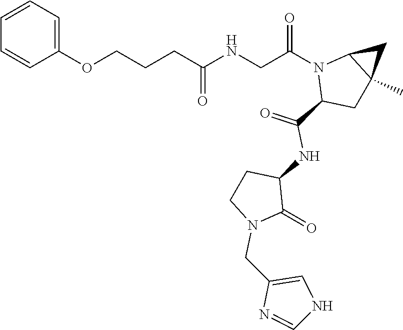 [SMILES: C[C@@]12C[C@@H]1N(C(=O)CNC(=O)CCCOc1ccccc1)[C@H](C(=O)N[C@@H]1CCN(Cc3c[nH]cn3)C1=O)C2] | (1S,3S,5S)-N-((R)-1-((1H- imidazol-4-yl)methyl)-2- oxopyrrolidin-3-yl)-5-methyl-2- ((4-phenoxybutanoyl)glycyl)-2- azabicyclo[3.1.0]hexane-3- carboxamide |
| 62 | 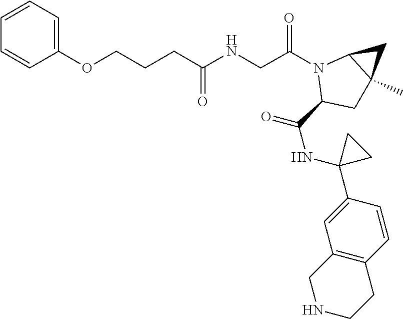 [SMILES: C[C@@]12C[C@@H]1N(C(=O)CNC(=O)CCCOc1ccccc1)[C@H](C(=O)NC1(c3ccc4c(c3)CNCC4)CC1)C2] | (1S,3S,5S)-5-methyl-2-((4- phenoxybutanoyl)glycyl)-N-(1- (1,2,3,4-tetrahydroisoquinolin-7- yl)cyclopropyl)-2- azabicyclo[3.1.0]hexane-3- carboxamide |
| 63 | 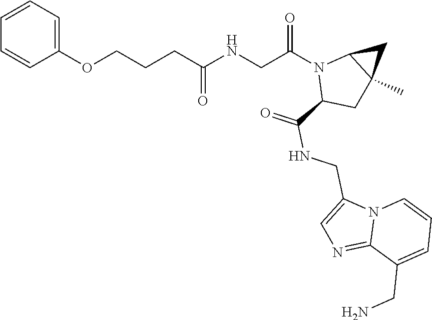 [SMILES: C[C@@]12C[C@@H]1N(C(=O)CNC(=O)CCCOc1ccccc1)[C@H](C(=O)NCc1cnc3c(CN)cccn13)C2] | (1S,3S,5S)-N-((8- (aminomethyl)imidazo[1,2- a]pyridin-3-yl)methyl)-5-methyl- 2-((4-phenoxybutanoyl)glycyl)-2- azabicyclo[3.1.0]hexane-3- carboxamide |
| 64 | 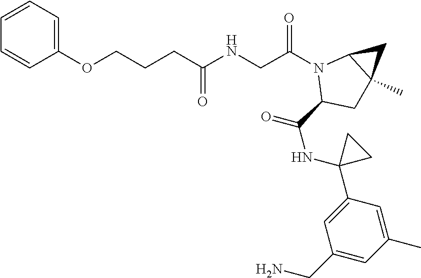 [SMILES: Cc1cc(CN)cc(C2(NC(=O)[C@@H]3C[C@]4(C)C[C@@H]4N3C(=O)CNC(=O)CCCOc3ccccc3)CC2)c1] | (1S,3S,5S)-N-(1-(3- (aminomethyl)-5- methylphenyl)cyclopropyl)-5- methyl-2-((4- phenoxybutanoyl)glycyl)-2- azabicyclo[3.1.0]hexane-3- carboxamide |
| 65 | 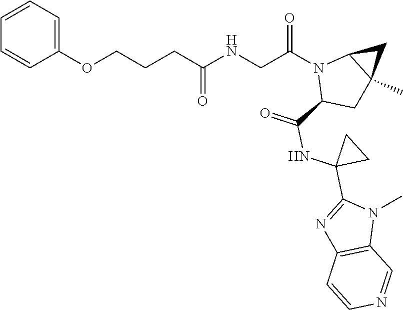 [SMILES: Cn1c(C2(NC(=O)[C@@H]3C[C@]4(C)C[C@@H]4N3C(=O)CNC(=O)CCCOc3ccccc3)CC2)nc2ccncc21] | (1S,3S,5S)-5-methyl-N-(1-(3- methyl-3H-imidazo[4,5-c]pyridin- 2-yl)cyclopropyl)-2-((4- phenoxybutanoyl)glycyl)-2- azabicyclo[3.1.0]hexane-3- carboxamide |
| 66 | 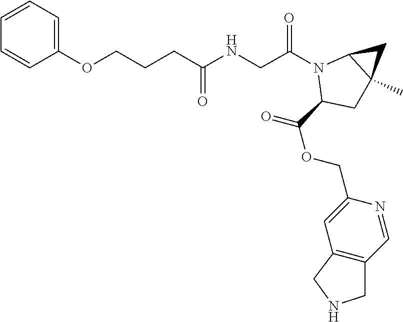 [SMILES: C[C@@]12C[C@@H]1N(C(=O)CNC(=O)CCCOc1ccccc1)[C@H](C(=O)OCc1cc3c(cn1)CNC3)C2] | (2,3-dihydro-1H-pyrrolo[3,4- c]pyridin-6-yl)methyl (1S,3S,5S)- 5-methyl-2-((4- phenoxybutanoyl)glycyl)-2- azabicyclo[3.1.0]hexane-3- carboxylate |
| 67 | 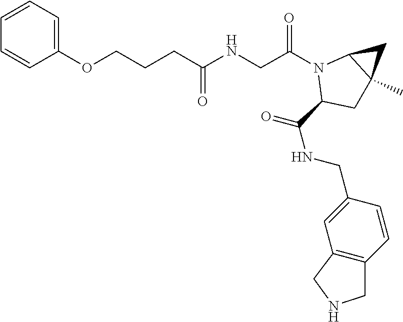 [SMILES: C[C@@]12C[C@@H]1N(C(=O)CNC(=O)CCCOc1ccccc1)[C@H](C(=O)NCc1ccc3c(c1)CNC3)C2] | (1S,3S,5S)-N-(isoindolin-5- ylmethyl)-5-methyl-2-((4- phenoxybutanoyl)glycyl)-2- azabicyclo[3.1.0]hexane-3- carboxamide |
| 68 | 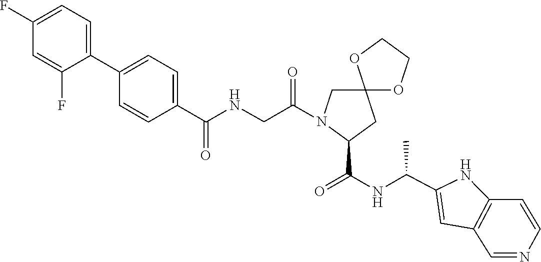 [SMILES: C[C@@H](NC(=O)[C@@H]1CC2(CN1C(=O)CNC(=O)c1ccc(-c3ccc(F)cc3F)cc1)OCCO2)c1cc2cnccc2[nH]1] | (S)-N-((R)-1-(1H-pyrrolo[3,2- c]pyridin-2-yl)ethyl)-7-((2′,4′- difluoro-[1,1′-biphenyl]-4- carbonyl)glycyl)-1,4-dioxa-7- azaspiro[4.4]nonane-8- carboxamide |
| 69 | 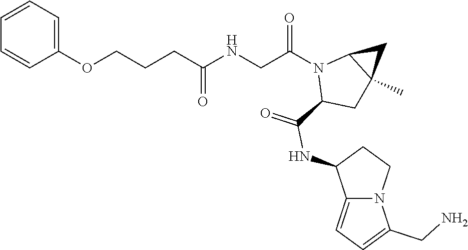 [SMILES: C[C@@]12C[C@@H]1N(C(=O)CNC(=O)CCCOc1ccccc1)[C@H](C(=O)N[C@H]1CCn3c(CN)ccc31)C2] | (1S,3S,5S)-N-((S)-5- (aminomethyl)-2,3-dihydro-1H- pyrrolizin-1-yl)-5-methyl-2-((4- phenoxybutanoyl)glycyl)-2- azabicyclo[3.1.0]hexane-3- carboxamide |
| 70 | 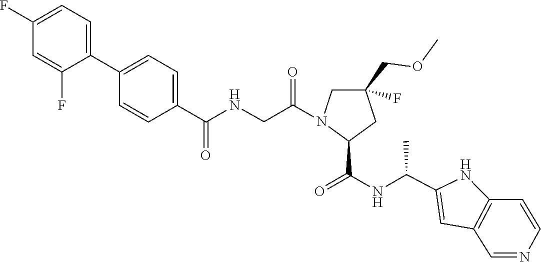 [SMILES: COC[C@@]1(F)C[C@@H](C(=O)N[C@H](C)c2cc3cnccc3[nH]2)N(C(=O)CNC(=O)c2ccc(-c3ccc(F)cc3F)cc2)C1] | (2S,4R)-N-((R)-1-(1H- pyrrolo[3,2-c]pyridin-2-yl)ethyl)- 1-((2′,4′-difluoro-[1,1′-biphenyl]- 4-carbonyl)glycyl)-4-fluoro-4- (methoxymethyl)pyrrolidine-2- carboxamide |
| 71 | 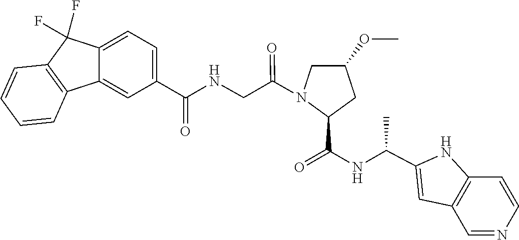 [SMILES: CO[C@@H]1C[C@@H](C(=O)N[C@H](C)c2cc3cnccc3[nH]2)N(C(=O)CNC(=O)c2ccc3c(c2)-c2ccccc2C3(F)F)C1] | (2S,4R)-N-((R)-1-(1H- pyrrolo[3,2-c]pyridin-2-yl)ethyl)- 1-((9,9-difluoro-9H-fluorene-3- carbonyl)glycyl)-4- methoxypyrrolidine-2- carboxamide |
| 72 | 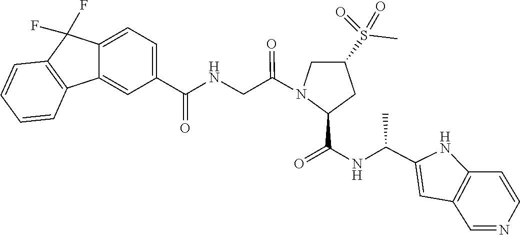 [SMILES: C[C@@H](NC(=O)[C@@H]1C[C@@H](S(C)(=O)=O)CN1C(=O)CNC(=O)c1ccc2c(c1)-c1ccccc1C2(F)F)c1cc2cnccc2[nH]1] | (2S,4R)-N-((R)-1-(1H- pyrrolo[3,2-c]pyridin-2-yl)ethyl)- 1-((9,9-difluoro-9H-fluorene-3- carbonyl)glycyl)-4- (methylsulfonyl)pyrrolidine-2- carboxamide |
| 73 | 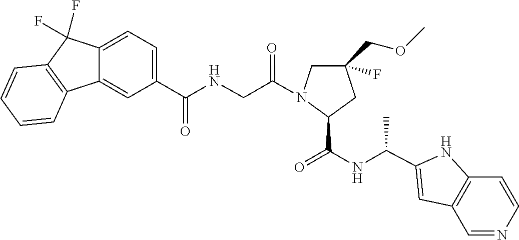 [SMILES: COC[C@@]1(F)C[C@@H](C(=O)N[C@H](C)c2cc3cnccc3[nH]2)N(C(=O)CNC(=O)c2ccc3c(c2)-c2ccccc2C3(F)F)C1] | (2S,4R)-N-((R)-1-(1H- pyrrolo[3,2-c]pyridin-2-yl)ethyl)- 1-((9,9-difluoro-9H-fluorene-3- carbonyl)glycyl)-4-fluoro-4- (methoxymethyl)pyrrolidine-2- carboxamide |
| 74 | 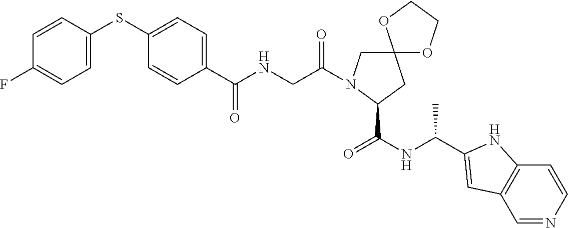 [SMILES: C[C@@H](NC(=O)[C@@H]1CC2(CN1C(=O)CNC(=O)c1ccc(Sc3ccc(F)cc3)cc1)OCCO2)c1cc2cnccc2[nH]1] | (S)-N-((R)-1-(1H-pyrrolo[3,2- c]pyridin-2-yl)ethyl)-7-((4-(4- fluorophenoxy)benzoyl)glycyl)- 1,4-dioxa-7-azaspiro[4.4]nonane- 8-carboxamide |
| 75 | 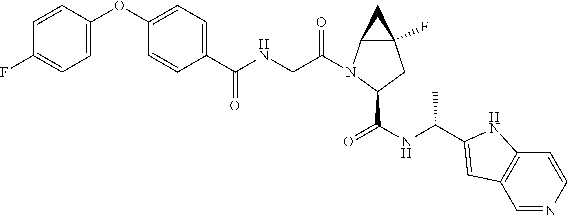 [SMILES: C[C@@H](NC(=O)[C@@H]1C[C@]2(F)C[C@@H]2N1C(=O)CNC(=O)c1ccc(Oc2ccc(F)cc2)cc1)c1cc2cnccc2[nH]1] | (1S,3S,5S)-N-((R)-1-(1H- pyrrolo[3,2-c]pyridin-2-yl)ethyl)- 2-((4-(4- fluorophenoxy)benzoyl)glycyl)-5- methyl-2- azabicyclo[3.1.0]hexane-3- carboxamide |
| 76 | 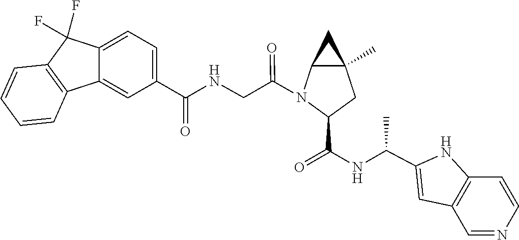 [SMILES: C[C@@H](NC(=O)[C@@H]1C[C@]2(C)C[C@@H]2N1C(=O)CNC(=O)c1ccc2c(c1)-c1ccccc1C2(F)F)c1cc2cnccc2[nH]1] | (1S,3S,5S)-N-((R)-1-(1H- pyrrolo[3,2-c]pyridin-2-yl)ethyl)- 2-((9,9-difluoro-9H-fluorene-3- carbonyl)glycyl)-5-methyl-2- azabicyclo[3.1.0]hexane-3- carboxamide |
| 77 | 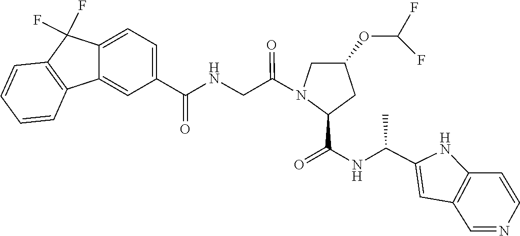 [SMILES: C[C@@H](NC(=O)[C@@H]1C[C@@H](OC(F)F)CN1C(=O)CNC(=O)c1ccc2c(c1)-c1ccccc1C2(F)F)c1cc2cnccc2[nH]1] | (2S,4R)-N-((R)-1-(1H- pyrrolo[3,2-c]pyridin-2-yl)ethyl)- 1-((9,9-difluoro-9H-fluorene-3- carbonyl)glycyl)-4- (difluoromethoxy)pyrrolidine-2- carboxamide |
| 78 | 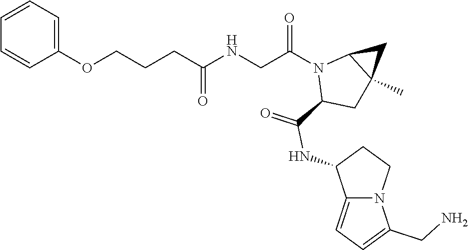 [SMILES: C[C@@]12C[C@@H]1N(C(=O)CNC(=O)CCCOc1ccccc1)[C@H](C(=O)N[C@@H]1CCn3c(CN)ccc31)C2] | (1S,3S,5S)-N-((R)-5- (aminomethyl)-2,3-dihydro-1H- pyrrolizin-1-yl)-5-methyl-2-((4- phenoxybutanoyl)glycyl)-2- azabicyclo[3.1.0]hexane-3- carboxamide |
| 79 | 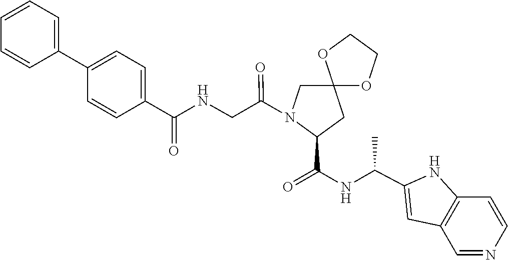 [SMILES: C[C@@H](NC(=O)[C@@H]1CC2(CN1C(=O)CNC(=O)c1ccc(-c3ccccc3)cc1)OCCO2)c1cc2cnccc2[nH]1] | (S)-N-((R)-1-(1H-pyrrolo[3,2- c]pyridin-2-yl)ethyl)-7-(([1,1′- biphenyl]-4-carbonyl)glycyl)-1,4- dioxa-7-azaspiro[4.4]nonane-8- carboxamide |
| 80 | 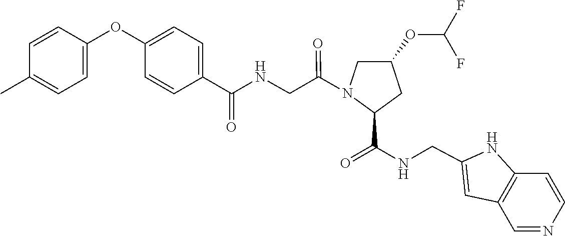 [SMILES: Cc1ccc(Oc2ccc(C(=O)NCC(=O)N3C[C@H](OC(F)F)C[C@H]3C(=O)NCc3cc4cnccc4[nH]3)cc2)cc1] | (2S,4R)-N-((R)-1-(1H- pyrrolo[3,2-c]pyridin-2-yl)ethyl)- 4-(difluoromethoxy)-1-((4-(p- tolyloxy)benzoyl)glycyl) pyrrolidine-2-carboxamide |
| 81 | 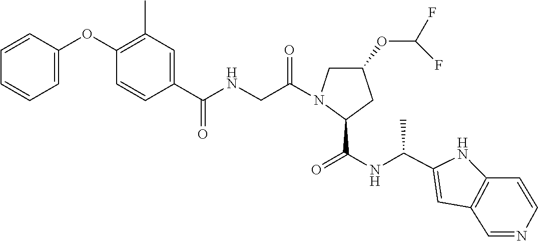 [SMILES: Cc1cc(C(=O)NCC(=O)N2C[C@H](OC(F)F)C[C@H]2C(=O)N[C@H](C)c2cc3cnccc3[nH]2)ccc1Oc1ccccc1] | (2S,4R)-N-((R)-1-(1H- pyrrolo[3,2-c]pyridin-2-yl)ethyl)- 4-(difluoromethoxy)-1-((3- methyl-4- phenoxybutanoyl)glycyl) pyrrolidine-2-carboxamide |
| 82 | 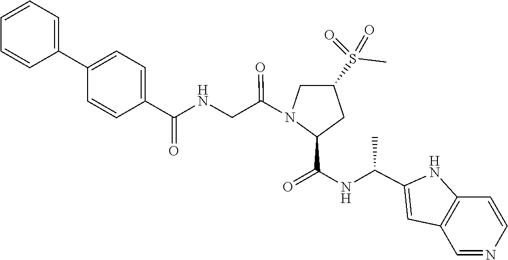 [SMILES: C[C@@H](NC(=O)[C@@H]1C[C@@H](S(C)(=O)=O)CN1C(=O)CNC(=O)c1ccc(-c2ccccc2)cc1)c1cc2cnccc2[nH]1] | (2S,4R)-N-((R)-1-(1H- pyrrolo[3,2-c]pyridin-2-yl)ethyl)- 1-(([1,1′-biphenyl]-4- carbonyl)glycyl)-4- (methylsulfonyl)pyrrolidine-2- carboxamide |
| 83 | 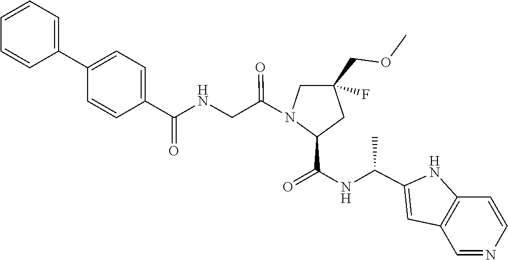 [SMILES: COC[C@@]1(F)C[C@@H](C(=O)N[C@H](C)c2cc3cnccc3[nH]2)N(C(=O)CNC(=O)c2ccc(-c3ccccc3)cc2)C1] | (2S,4R)-N-((R)-1-(1H- pyrrolo[3,2-c]pyridin-2-yl)ethyl)- 1-(([1,1′-biphenyl]-4- carbonyl)glycyl)-4-fluoro-4- (methoxymethyl)pyrrolidine-2- carboxamide |
| 84 | 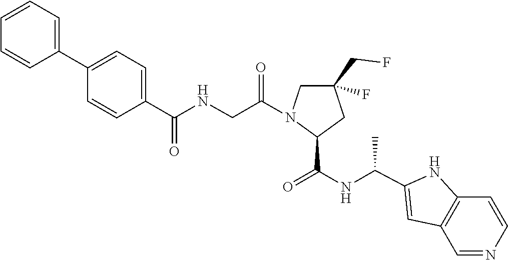 [SMILES: C[C@@H](NC(=O)[C@@H]1C[C@](F)(CF)CN1C(=O)CNC(=O)c1ccc(-c2ccccc2)cc1)c1cc2cnccc2[nH]1] | (2S,4R)-N-((R)-1-(1H- pyrrolo[3,2-c]pyridin-2-yl)ethyl)- 1-(([1,1′-biphenyl]-4- carbonyl)glycyl)-4-fluoro-4- (fluoromethyl)pyrrolidine-2- carboxamide |
| 85 | 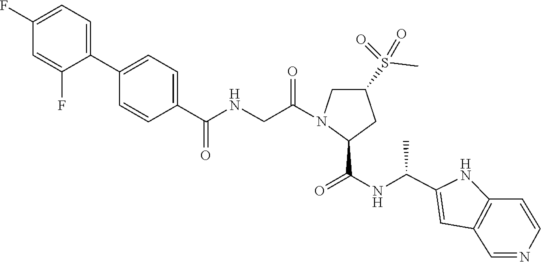 [SMILES: C[C@@H](NC(=O)[C@@H]1C[C@@H](S(C)(=O)=O)CN1C(=O)CNC(=O)c1ccc(-c2ccc(F)cc2F)cc1)c1cc2cnccc2[nH]1] | (2S,4R)-N-((R)-1-(1H- pyrrolo[3,2-c]pyridin-2-yl)ethyl)- 1-((2′,4′-difluoro-[1,1′-biphenyl]- 4-carbonyl)glycyl)-4- (methylsulfonyl)pyrrolidine-2- carboxamide |
| 86 | 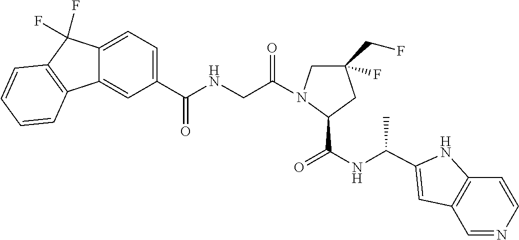 [SMILES: C[C@@H](NC(=O)[C@@H]1C[C@](F)(CF)CN1C(=O)CNC(=O)c1ccc2c(c1)-c1ccccc1C2(F)F)c1cc2cnccc2[nH]1] | (2S,4R)-N-((R)-1-(1H- pyrrolo[3,2-c]pyridin-2-yl)ethyl)- 1-((9,9-difluoro-9H-fluorene-3- carbonyl)glycyl)-4-fluoro-4- (fluoromethyl)pyrrolidine-2- carboxamide |
| 87 | 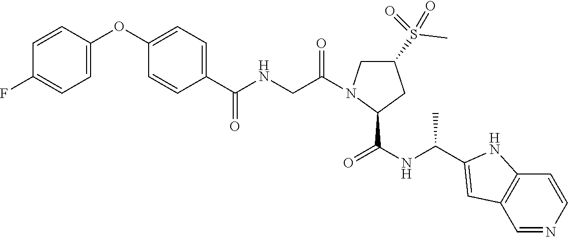 [SMILES: C[C@@H](NC(=O)[C@@H]1C[C@@H](S(C)(=O)=O)CN1C(=O)CNC(=O)c1ccc(Oc2ccc(F)cc2)cc1)c1cc2cnccc2[nH]1] | (2S,4R)-N-((R)-1-(1H- pyrrolo[3,2-c]pyridin-2-yl)ethyl)- 1-((4-(4- fluorophenoxy)benzoyl)glycyl)-4- (methylsulfonyl)pyrrolidine-2- carboxamide |
| 88 | 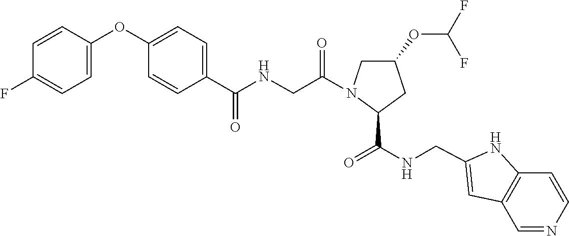 [SMILES: O=C(NCC(=O)N1C[C@H](OC(F)F)C[C@H]1C(=O)NCc1cc2cnccc2[nH]1)c1ccc(Oc2ccc(F)cc2)cc1] | (2S,4R)-N-((R)-1-(1H- pyrrolo[3,2-c]pyridin-2-yl)ethyl)- 4-(difluoromethoxy)-1-((4-(4- fluorophenoxy)benzoyl)glycyl) pyrrolidine-2-carboxamide |
| 89 | 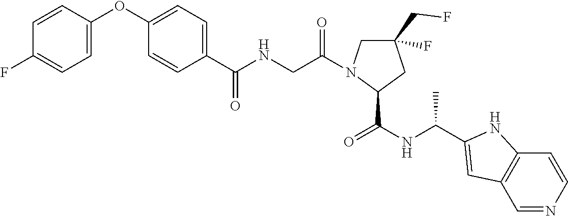 [SMILES: C[C@@H](NC(=O)[C@@H]1C[C@](F)(CF)CN1C(=O)CNC(=O)c1ccc(Oc2ccc(F)cc2)cc1)c1cc2cnccc2[nH]1] | (2S,4R)-N-((R)-1-(1H- pyrrolo[3,2-c]pyridin-2-yl)ethyl)- 4-fluoro-4-(fluoromethyl)-1-((4- (4- fluorophenoxy)benzoyl)glycyl) pyrrolidine-2-carboxamide |
| 90 | 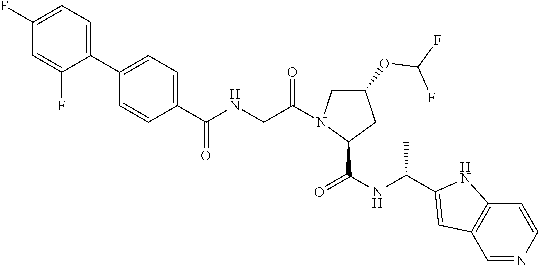 [SMILES: C[C@@H](NC(=O)[C@@H]1C[C@@H](OC(F)F)CN1C(=O)CNC(=O)c1ccc(-c2ccc(F)cc2F)cc1)c1cc2cnccc2[nH]1] | (2S,4R)-N-((R)-1-(1H- pyrrolo[3,2-c]pyridin-2-yl)ethyl)- 1-((2′,4′′-difluoro-[1,1′-biphenyl]- 4-carbonyl)glycyl)-4- (difluoromethoxy)pyrrolidine-2- carboxamide |
| 91 | 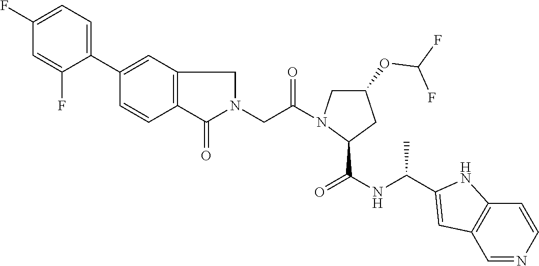 [SMILES: C[C@@H](NC(=O)[C@@H]1C[C@@H](OC(F)F)CN1C(=O)CN1Cc2cc(-c3ccc(F)cc3F)ccc2C1=O)c1cc2cnccc2[nH]1] | (2S,4R)-N-((R)-1-(1H- pyrrolo[3,2-c]pyridin-2-yl)ethyl)- 4-(difluoromethoxy)-1-(2-(5-(2,4- difluorophenyl)-1-oxoisoindolin- 2-yl)acetyl)pyrrolidine-2- carboxamide |
| 92 | 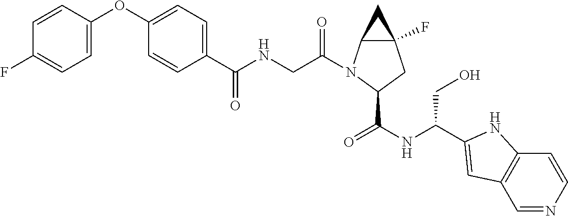 [SMILES: O=C(NCC(=O)N1[C@H]2C[C@@]2(F)C[C@H]1C(=O)N[C@H](CO)c1cc2cnccc2[nH]1)c1ccc(Oc2ccc(F)cc2)cc1] | (1S,3S,5S)-2-((4-(4- fluorophenoxy)benzoyl)glycyl)- N-((S)-2-hydroxy-1-(1H- pyrrolo[3,2-c]pyridin-2-yl)ethyl)- 5-methyl-2- azabicyclo[3.1.0]hexane-3- carboxamide |
| 93 | 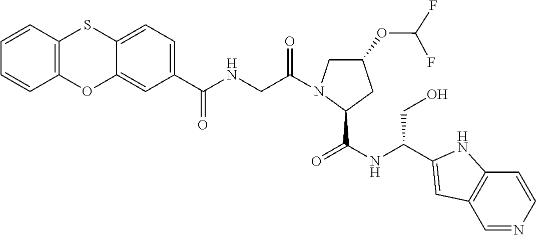 [SMILES: O=C(NCC(=O)N1C[C@H](OC(F)F)C[C@H]1C(=O)N[C@H](CO)c1cc2cnccc2[nH]1)c1ccc2c(c1)Oc1ccccc1S2] | (2S,4R)-4-(difluoromethoxy)- N-((S)-2-hydroxy-1-(1H- pyrrolo[3,2-c]pyridin-2-yl)ethyl)- 1-((phenoxathiine-3- carbonyl)glycyl)pyrrolidine-2- carboxamide |
| 94 | 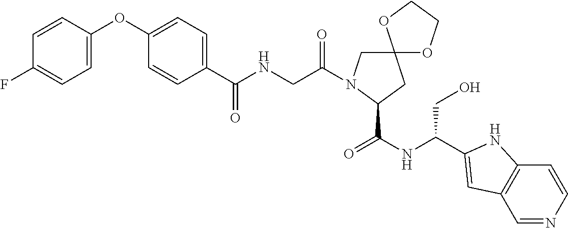 [SMILES: O=C(NCC(=O)N1CC2(C[C@H]1C(=O)N[C@H](CO)c1cc3cnccc3[nH]1)OCCO2)c1ccc(Oc2ccc(F)cc2)cc1] | (S)-7-((4-(4- fluorophenoxy)benzoyl)glycyl)- N-((S)-2-hydroxy-1-(1H- pyrrolo[3,2-c]pyridin-2-yl)ethyl)- 1,4-dioxa-7-azaspiro[4.4]nonane- 8-carboxamide |
| 95 | 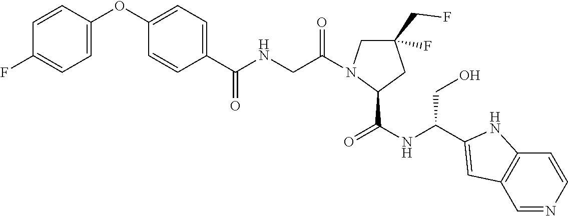 [SMILES: O=C(NCC(=O)N1C[C@@](F)(CF)C[C@H]1C(=O)N[C@H](CO)c1cc2cnccc2[nH]1)c1ccc(Oc2ccc(F)cc2)cc1] | (2S,4R)-4-fluoro-4- (fluoromethyl)-1-((4-(4- fluorophenoxy)benzoyl)glycyl)- N-((S)-2-hydroxy-1-(1H- pyrrolo[3,2-c]pyridin-2- yl)ethyl)pyrrolidine-2-carboxamide |
| 96 | 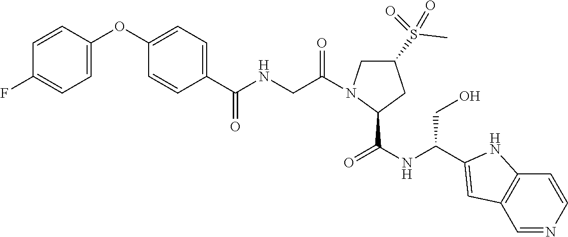 [SMILES: CS(=O)(=O)[C@@H]1C[C@@H](C(=O)N[C@H](CO)c2cc3cnccc3[nH]2)N(C(=O)CNC(=O)c2ccc(Oc3ccc(F)cc3)cc2)C1] | (2S,4R)-1-((4-(4- fluorophenoxy)benzoyl)glycyl)- N-((S)-2-hydroxy-1-(1H- pyrrolo[3,2-c]pyridin-2-yl)ethyl)- 4-(methylsulfonyl) pyrrolidine-2-carboxamide |
| 97 | 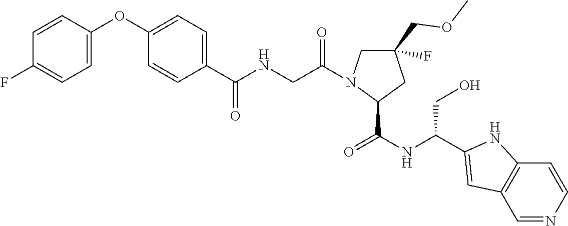 [SMILES: COC[C@@]1(F)C[C@@H](C(=O)N[C@H](CO)c2cc3cnccc3[nH]2)N(C(=O)CNC(=O)c2ccc(Oc3ccc(F)cc3)cc2)C1] | (2S,4R)-4-fluoro-1-((4-(4- fluorophenoxy)benzoyl)glycyl)- N-((S)-2-hydroxy-1-(1H- pyrrolo[3,2-c]pyridin-2-yl)ethyl)- 4-(methoxymethyl) pyrrolidine-2-carboxamide |
| 98 | 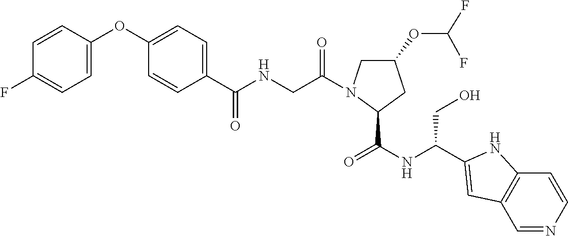 [SMILES: O=C(NCC(=O)N1C[C@H](OC(F)F)C[C@H]1C(=O)N[C@H](CO)c1cc2cnccc2[nH]1)c1ccc(Oc2ccc(F)cc2)cc1] | (2S,4R)-4-(difluoromethoxy)-1- ((4-(4- fluorophenoxy)benzoyl)glycyl)- N-((S)-2-hydroxy-1-(1H- pyrrolo[3,2-c]pyridin-2- yl)ethyl)pyrrolidine-2-carboxamide |
| 99 | 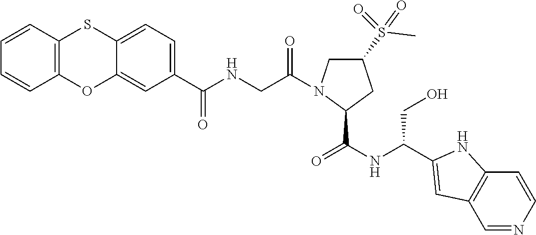 [SMILES: CS(=O)(=O)[C@@H]1C[C@@H](C(=O)N[C@H](CO)c2cc3cnccc3[nH]2)N(C(=O)CNC(=O)c2ccc3c(c2)Oc2ccccc2S3)C1] | (2S,4R)-N-((S)-2-hydroxy-1-(1H- pyrrolo[3,2-c]pyridin-2-yl)ethyl)- 4-(methylsulfonyl)-1- ((phenoxathiine-3- carbonyl)glycyl) pyrrolidine-2-carboxamide |
| 100 | 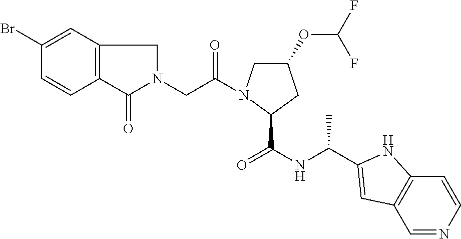 [SMILES: C[C@@H](NC(=O)[C@@H]1C[C@@H](OC(F)F)CN1C(=O)CN1Cc2cc(Br)ccc2C1=O)c1cc2cnccc2[nH]1] | (2S,4R)-N-((R)-1-(1H- pyrrolo[3,2-c]pyridin-2-yl)ethyl)- 1-(2-(5-bromo-1-oxoisoindolin-2- yl)acetyl)-4- (difluoromethoxy)pyrrolidine-2- carboxamide |
| 101 | 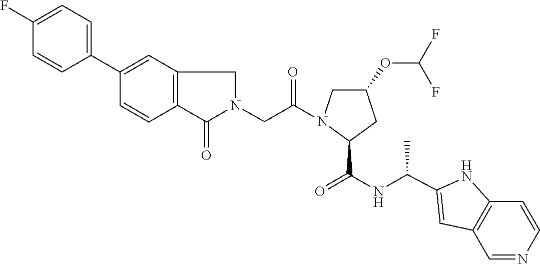 [SMILES: C[C@@H](NC(=O)[C@@H]1C[C@@H](OC(F)F)CN1C(=O)CN1Cc2cc(-c3ccc(F)cc3)ccc2C1=O)c1cc2cnccc2[nH]1] | (2S,4R)-N-((R)-1-(1H- pyrrolo[3,2-c]pyridin-2-yl)ethyl)- 4-(difluoromethoxy)-1-(2-(5-(4- fluorophenyl)-1-oxoisoindolin-2- yl)acetyl)pyrrolidine-2- carboxamide |
| 102 | 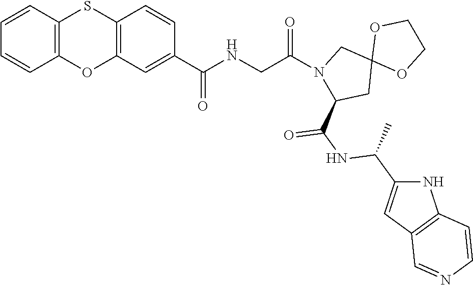 [SMILES: C[C@@H](NC(=O)[C@@H]1CC2(CN1C(=O)CNC(=O)c1ccc3c(c1)Oc1ccccc1S3)OCCO2)c1cc2cnccc2[nH]1] | (S)-N-((S)-1-(1H-pyrrolo[3,2- c]pyridin-2-yl)ethyl)-7- ((phenoxathiine-3- carbonyl)glycyl)-1,4-dioxa-7- azaspiro[4.4]nonane-8- carboxamide |
| 103 | 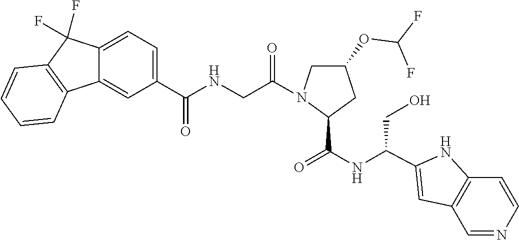 [SMILES: O=C(NCC(=O)N1C[C@H](OC(F)F)C[C@H]1C(=O)N[C@H](CO)c1cc2cnccc2[nH]1)c1ccc2c(c1)-c1ccccc1C2(F)F] | (2S,4R)-1-((9,9-difluoro-9H- fluorene-3-carbonyl)glycyl)-4- (difluoromethoxy)-N-((S)-2- hydroxy-1-(1H-pyrrolo[3,2- c]pyridin-2-yl)ethyl)pyrrolidine- 2-carboxamide |
| 104 | 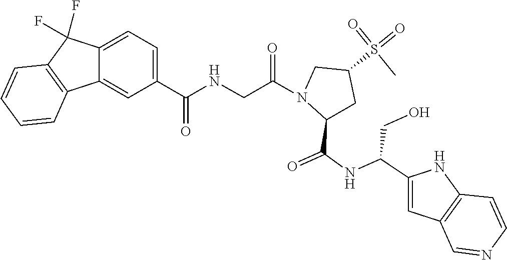 [SMILES: CS(=O)(=O)[C@@H]1C[C@@H](C(=O)N[C@H](CO)c2cc3cnccc3[nH]2)N(C(=O)CNC(=O)c2ccc3c(c2)-c2ccccc2C3(F)F)C1] | (2S,4R)-1-((9,9-difluoro-9H- fluorene-3-carbonyl)glycyl)-N- ((S)-2-hydroxy-1-(1H- pyrrolo[3,2-c]pyridin-2-yl)ethyl)- 4-(methylsulfonyl) pyrrolidine-2-carboxamide |
| 105 | 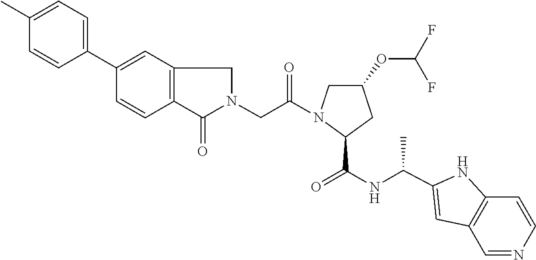 [SMILES: Cc1ccc(-c2ccc3c(c2)CN(CC(=O)N2C[C@H](OC(F)F)C[C@H]2C(=O)N[C@H](C)c2cc4cnccc4[nH]2)C3=O)cc1] | (2S,4R)-N-((R)-1-(1H- pyrrolo[3,2-c]pyridin-2-yl)ethyl)- 4-(difluoromethoxy)-1-(2-(1-oxo- 5-(p-tolyl)isoindolin-2- yl)acetyl)pyrrolidine-2- carboxamide |
| 106 | 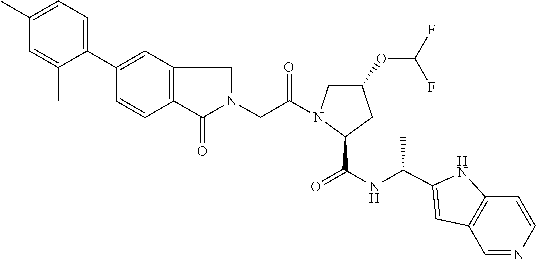 [SMILES: Cc1ccc(-c2ccc3c(c2)CN(CC(=O)N2C[C@H](OC(F)F)C[C@H]2C(=O)N[C@H](C)c2cc4cnccc4[nH]2)C3=O)c(C)c1] | (2S,4R)-N-((R)-1-(1H- pyrrolo[3,2-c]pyridin-2-yl)ethyl)- 4-(difluoromethoxy)-1-(2-(5-(2,4- dimethylphenyl)-1-oxoisoindolin- 2-yl)acetyl)pyrrolidine-2- carboxamide |
| 107 | 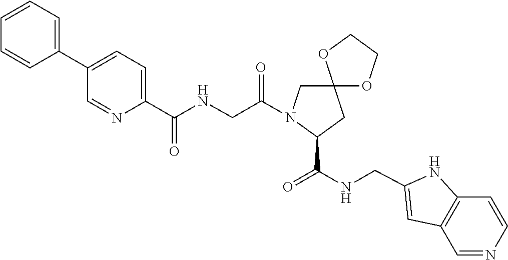 [SMILES: O=C(NCC(=O)N1CC2(C[C@H]1C(=O)NCc1cc3cnccc3[nH]1)OCCO2)c1ccc(-c2ccccc2)cn1] | (S)-N-((1H-pyrrolo[3,2-c]pyridin- 2-yl)methyl)-7-((5- phenylpicolinoyl)glycyl)-1,4- dioxa-7-azaspiro[4.4]nonane-8- carboxamide |
| 108 | 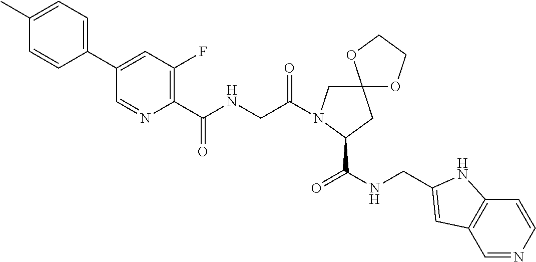 [SMILES: Cc1ccc(-c2cnc(C(=O)NCC(=O)N3CC4(C[C@H]3C(=O)NCc3cc5cnccc5[nH]3)OCCO4)c(F)c2)cc1] | (S)-N-((1H-pyrrolo[3,2-c]pyridin- 2-yl)methyl)-7-((3-fluoro-5-(p- tolyl)picolinoyl)glycyl)-1,4-dioxa- 7-azaspiro[4.4]nonane-8- carboxamide |
| 109 | 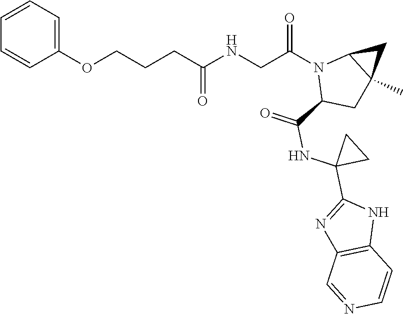 [SMILES: C[C@@]12C[C@@H]1N(C(=O)CNC(=O)CCCOc1ccccc1)[C@H](C(=O)NC1(c3nc4cnccc4[nH]3)CC1)C2] | (1S,3S,5S)-N-(1-(1H- imidazo[4,5-c]pyridin-2- yl)cyclopropyl)-5-methyl-2-((4- phenoxybutanoyl)glycyl)-2- azabicyclo[3.1.0]hexane-3- carboxamide |
| 110 | 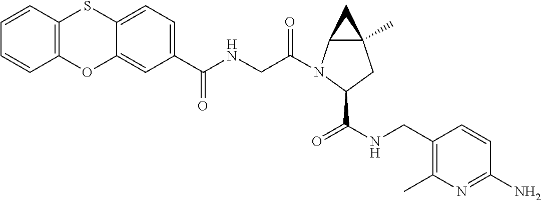 [SMILES: Cc1nc(N)ccc1CNC(=O)[C@@H]1C[C@]2(C)C[C@@H]2N1C(=O)CNC(=O)c1ccc2c(c1)Oc1ccccc1S2] | (1S,3S,5S)-N-((6-amino-2- methylpyridin-3-yl)methyl)-5- methyl-2-((phenoxathiine-3- carbonyl)glycyl)-2- azabicyclo[3.1.0]hexane-3- carboxamide |
| 111 | 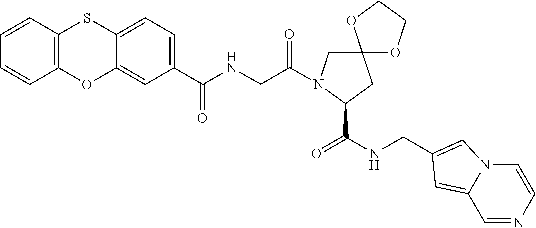 [SMILES: O=C(NCC(=O)N1CC2(C[C@H]1C(=O)NCc1cc3cnccn3c1)OCCO2)c1ccc2c(c1)Oc1ccccc1S2] | (S)-7-((phenoxathine-3- carbonyl)glycyl)-N-(pyrrolo[1,2- a]pyrazin-7-ylmethyl)-1,4-dioxa- 7-azaspiro[4.4]nonane-8- carboxamide |
| 112 | 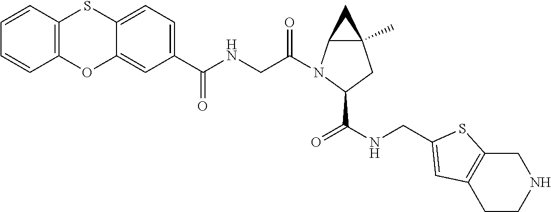 [SMILES: C[C@@]12C[C@@H]1N(C(=O)CNC(=O)c1ccc3c(c1)Oc1ccccc1S3)[C@H](C(=O)NCc1cc3c(s1)CNCC3)C2] | (1S,3S,5S)-5-methyl-2- ((phenoxathiine-3- carbonyl)glycyl)-N-((4,5,6,7- tetrahydrothieno[2,3-c]pyridin-2- yl)methyl)-2- azabicyclo[3.1.0]hexane-3- carboxamide |
| 113 | 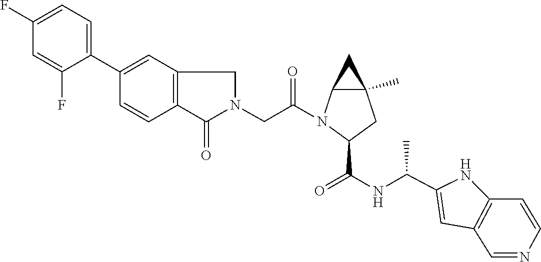 [SMILES: C[C@@H](NC(=O)[C@@H]1C[C@]2(C)C[C@@H]2N1C(=O)CN1Cc2cc(-c3ccc(F)cc3F)ccc2C1=O)c1cc2cnccc2[nH]1] | (1S,3S,5S)-N-((R)-1-(1H- pyrrolo[3,2-c]pyridin-2-yl)ethyl)- 2-(2-(5-(2,4-difluorophenyl)-1- oxoisoindolin-2-yl)acetyl)-5- methyl-2- azabicyclo[3.1.0]hexane-3- carboxamide |
| 114 | 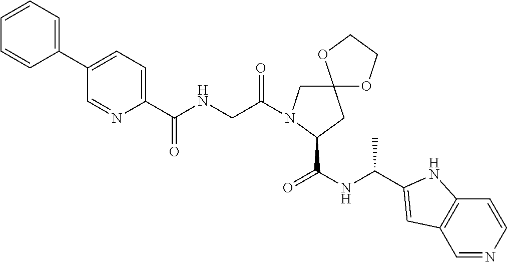 [SMILES: C[C@@H](NC(=O)[C@@H]1CC2(CN1C(=O)CNC(=O)c1ccc(-c3ccccc3)cn1)OCCO2)c1cc2cnccc2[nH]1] | (S)-N-((R)-1-(1H-pyrrolo[3,2- c]pyridin-2-yl)ethyl)-7-((5- phenylpicolinoyl)glycyl)-1,4- dioxa-7-azaspiro[4.4]nonane-8- carboxamide |
| 115 | 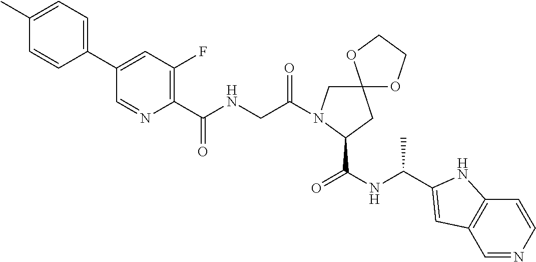 [SMILES: Cc1ccc(-c2cnc(C(=O)NCC(=O)N3CC4(C[C@H]3C(=O)N[C@H](C)c3cc5cnccc5[nH]3)OCCO4)c(F)c2)cc1] | (S)-N-((R)-1-(1H-pyrrolo[3,2- c]pyridin-2-yl)ethyl)-7-((3-fluoro- 5-(p-tolyl)picolinoyl)glycyl)-1,4- dioxa-7-azaspiro[4.4]nonane-8- carboxamide |
| 116 | 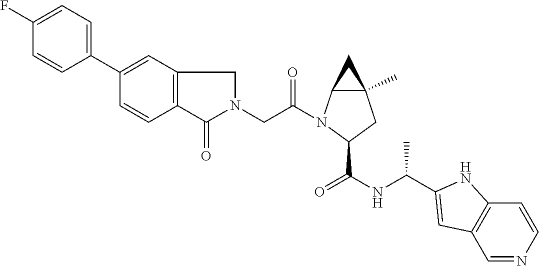 [SMILES: C[C@@H](NC(=O)[C@@H]1C[C@]2(C)C[C@@H]2N1C(=O)CN1Cc2cc(-c3ccc(F)cc3)ccc2C1=O)c1cc2cnccc2[nH]1] | (1S,3S,5S)-N-((R)-1-(1H- pyrrolo[3,2-c]pyridin-2-yl)ethyl)- 2-(2-(5-(4-fluorophenyl)-1- oxoisoindolin-2-yl)acetyl)-5- methyl-2- azabicyclo[3.1.0]hexane-3- carboxamide |
| 117 | 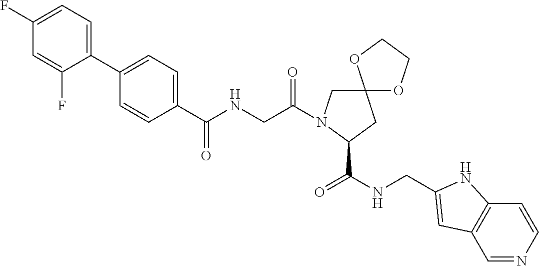 [SMILES: O=C(NCC(=O)N1CC2(C[C@H]1C(=O)NCc1cc3cnccc3[nH]1)OCCO2)c1ccc(-c2ccc(F)cc2F)cc1] | (S)-N-((1H-pyrrolo[3,2-c]pyridin- 2-yl)methyl)-7-((2′,4′-difluoro- [1,1′-biphenyl]-4- carbonyl)glycyl)-1,4-dioxa-7- azaspiro[4.4]nonane-8- carboxamide |
| 118 | 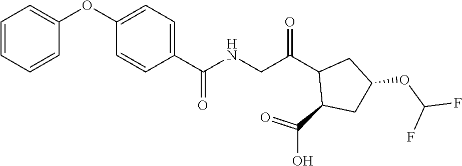 [SMILES: O=C(NCC(=O)C1C[C@H](OC(F)F)C[C@H]1C(=O)O)c1ccc(Oc2ccccc2)cc1] | (2S,4R)-4-(difluoromethoxy)-1- ((4- phenoxybenzoyl)glycyl) pyrrolidine-2-carboxylic acid |
| 119 | 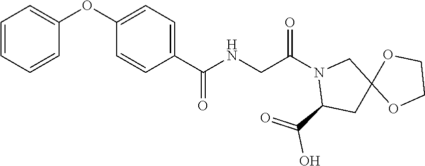 [SMILES: O=C(NCC(=O)N1CC2(C[C@H]1C(=O)O)OCCO2)c1ccc(Oc2ccccc2)cc1] | (S)-7-((4- phenoxybenzoyl)glycyl)-1,4- dioxa-7-azaspiro[4.4]nonane-8- carboxylic acid |
| 120 | 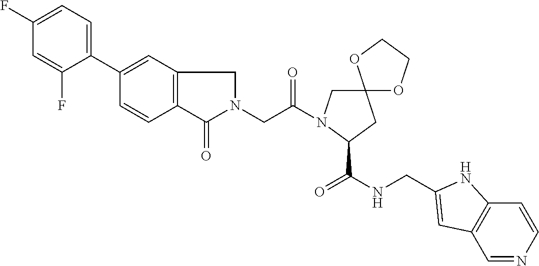 [SMILES: O=C(NCc1cc2cnccc2[nH]1)[C@@H]1CC2(CN1C(=O)CN1Cc3cc(-c4ccc(F)cc4F)ccc3C1=O)OCCO2] | (S)-N-((1H-pyrrolo[3,2-c]pyridin- 2-yl)methyl)-7-(2-(5-(2,4- difluorophenyl)-1-oxoisoindolin- 2-yl)acetyl)-1,4-dioxa-7- azaspiro[4.4]nonane-8- carboxamide |
| 121 | 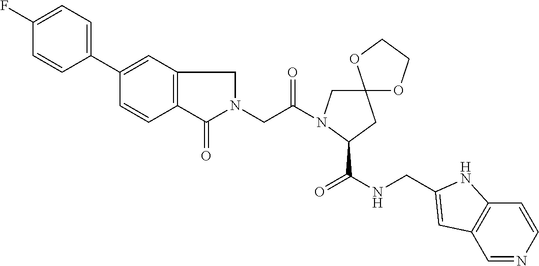 [SMILES: O=C(NCc1cc2cnccc2[nH]1)[C@@H]1CC2(CN1C(=O)CN1Cc3cc(-c4ccc(F)cc4)ccc3C1=O)OCCO2] | (S)-N-((1H-pyrrolo[3,2-c]pyridin- 2-yl)methyl)-7-(2-(5-(4- fluorophenyl)-1-oxoisoindolin-2- yl)acetyl)-1,4-dioxa-7- azaspiro[4.4]nonane-8- carboxamide |
| 122 | 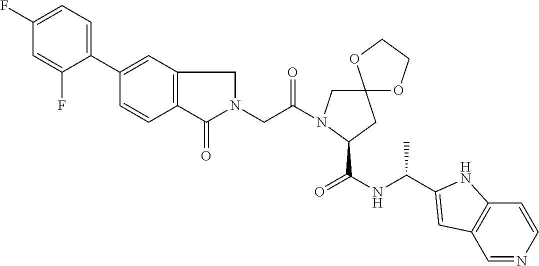 [SMILES: C[C@@H](NC(=O)[C@@H]1CC2(CN1C(=O)CN1Cc3cc(-c4ccc(F)cc4F)ccc3C1=O)OCCO2)c1cc2cnccc2[nH]1] | (S)-N-((R)-1-(1H-pyrrolo[3,2- c]pyridin-2-yl)ethyl)-7-(2-(5-(2,4- difluorophenyl)-1-oxoisoindolin- 2-yl)acetyl)-1,4-dioxa-7- azaspiro[4.4]nonane-8- carboxamide |
| 123 | 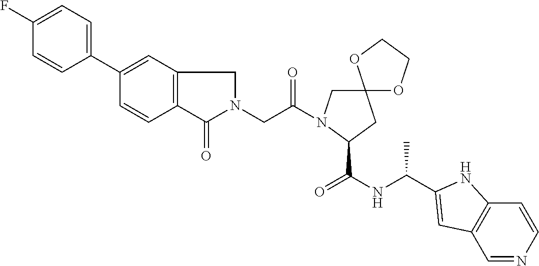 [SMILES: C[C@@H](NC(=O)[C@@H]1CC2(CN1C(=O)CN1Cc3cc(-c4ccc(F)cc4)ccc3C1=O)OCCO2)c1cc2cnccc2[nH]1] | (S)-N-((R)-1-(1H-pyrrolo[3,2- c]pyridin-2-yl)ethyl)-7-(2-(5-(4- fluorophenyl)-1-oxoisoindolin-2- yl)acetyl)-1,4-dioxa-7- azaspiro[4.4]nonane-8- carboxamide |
| 124 | 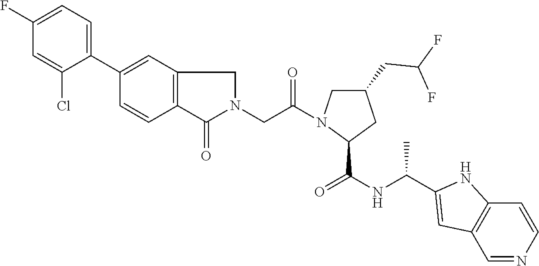 [SMILES: C[C@@H](NC(=O)[C@@H]1C[C@@H](CC(F)F)CN1C(=O)CN1Cc2cc(-c3ccc(F)cc3Cl)ccc2C1=O)c1cc2cnccc2[nH]1] | (2S,4R)-N-((R)-1-(1H- pyrrolo[3,2-c]pyridin-2-yl)ethyl)- 1-(2-(5-(2-chloro-4- fluorophenyl)-1-oxoisoindolin-2- yl)acetyl)-4- (difluoromethoxy)pyrrolidine-2- carboxamide |
| 125 | 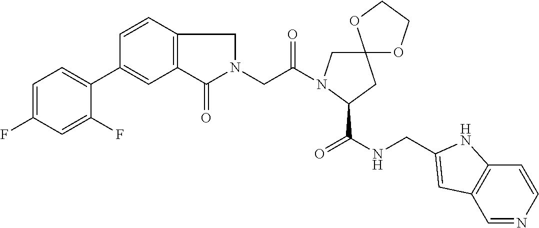 [SMILES: O=C(NCc1cc2cnccc2[nH]1)[C@@H]1CC2(CN1C(=O)CN1Cc3ccc(-c4ccc(F)cc4F)cc3C1=O)OCCO2] | (S)-N-((1H-pyrrolo[3,2-c]pyridin- 2-yl)methyl)-7-(2-(6-(2,4- difluorophenyl)-1-oxoisoindolin- 2-yl)acetyl)-1,4-dioxa-7- azaspiro[4.4]nonane-8- carboxamide |
| 126 | 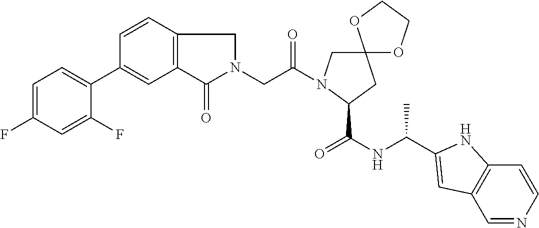 [SMILES: C[C@@H](NC(=O)[C@@H]1CC2(CN1C(=O)CN1Cc3ccc(-c4ccc(F)cc4F)cc3C1=O)OCCO2)c1cc2cnccc2[nH]1] | (S)-N-((R)-1-(1H-pyrrolo[3,2- c]pyridin-2-yl)ethyl)-7-(2-(6-(2,4- difluorophenyl)-1-oxoisoindolin- 2-yl)acetyl)-1,4-dioxa-7- azaspiro[4.4]nonane-8- carboxamide |
| 127 | 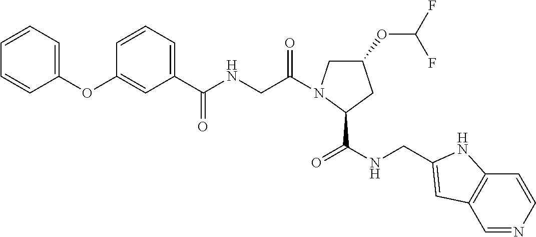 [SMILES: O=C(NCC(=O)N1C[C@H](OC(F)F)C[C@H]1C(=O)NCc1cc2cnccc2[nH]1)c1cccc(Oc2ccccc2)c1] | (2S,4R)-N-((1H-pyrrolo[3,2- c]pyridin-2-yl)methyl)-4- (difluoromethoxy)-1-((3- phenoxybenzoyl)glycyl) pyrrolidine-2-carboxamide |
| 128 | 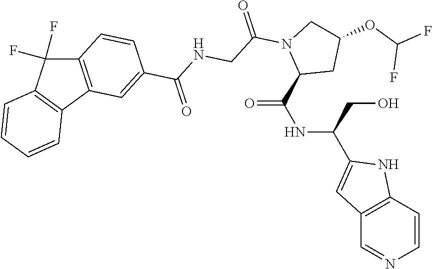 [SMILES: O=C(NCC(=O)N1C[C@H](OC(F)F)C[C@H]1C(=O)N[C@@H](CO)c1cc2cnccc2[nH]1)c1ccc2c(c1)-c1ccccc1C2(F)F] | (2S,4R)-1-((9,9-difluoro-9H- fluorene-3-carbonyl)glycyl)-4- (difluoromethoxy)-N-((R)-2- hydroxy-1-(1H-pyrrolo[3,2- c]pyridin-2-yl)ethyl)pyrrolidine- 2-carboxamide |
| 129 | 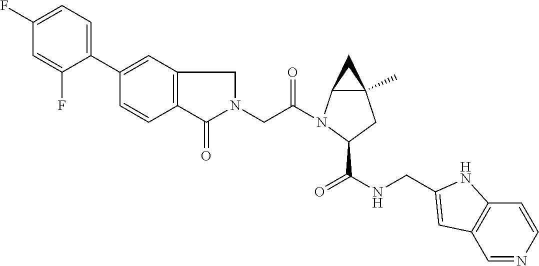 [SMILES: C[C@@]12C[C@@H]1N(C(=O)CN1Cc3cc(-c4ccc(F)cc4F)ccc3C1=O)[C@H](C(=O)NCc1cc3cnccc3[nH]1)C2] | (1S,3S,5S)-N-((1H-pyrrolo[3,2- c]pyridin-2-yl)methyl)-2-(2-(5- (2,4-difluorophenyl)-1- oxoisoindolin-2-yl)acetyl)-5- methyl-2- azabicyclo[3.1.0]hexane-3- carboxamide |
| 130 | 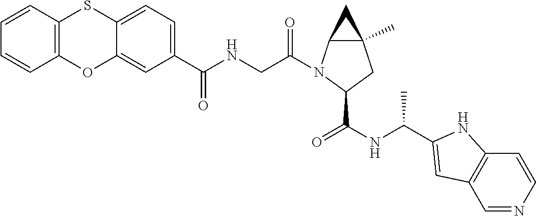 [SMILES: C[C@@H](NC(=O)[C@@H]1C[C@]2(C)C[C@@H]2N1C(=O)CNC(=O)c1ccc2c(c1)Oc1ccccc1S2)c1cc2cnccc2[nH]1] | (1S,3S,5S)-N-((R)-1-(1H- pyrrolo[3,2-c]pyridin-2-yl)ethyl)- 5-methyl-2-((phenoxathiine-3- carbonyl)glycyl)-2- azabicyclo[3.1.0]hexane-3- carboxamide |
| 131 | 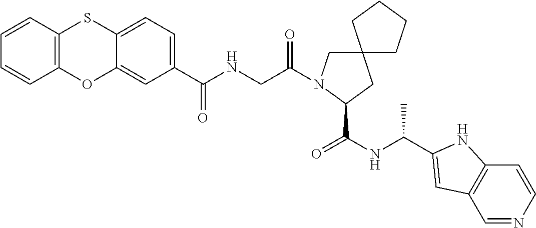 [SMILES: C[C@@H](NC(=O)[C@@H]1CC2(CCCC2)CN1C(=O)CNC(=O)c1ccc2c(c1)Oc1ccccc1S2)c1cc2cnccc2[nH]1] | (S)-N-((R)-1-(1H-pyrrolo[3,2- c]pyridin-2-yl)ethyl)-2- ((phenoxathiine-3- carbonyl)glycyl)-2- azaspiro[4.4]nonane-8- carboxamide |
| 132 | 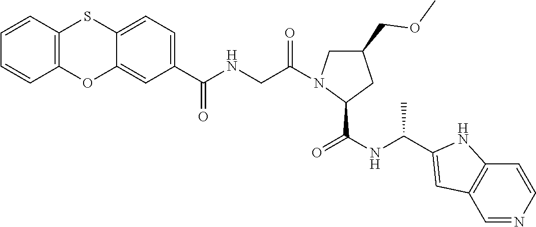 [SMILES: COC[C@H]1C[C@@H](C(=O)N[C@H](C)c2cc3cnccc3[nH]2)N(C(=O)CNC(=O)c2ccc3c(c2)Oc2ccccc2S3)C1] | (2S,4S)-N-((R)-1-(1H- pyrrolo[3,2-c]pyridin-2-yl)ethyl)- 4-(methoxymethyl)-1- ((phenoxathiine-3- carbonyl)glycyl)pyrrolidine-2- carboxamide |
| 133 | 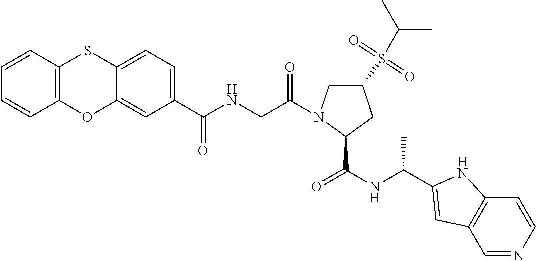 [SMILES: CC(C)S(=O)(=O)[C@@H]1C[C@@H](C(=O)N[C@H](C)c2cc3cnccc3[nH]2)N(C(=O)CNC(=O)c2ccc3c(c2)Oc2ccccc2S3)C1] | (2S,4R)-N-((R)-1-(1H- pyrrolo[3,2-c]pyridin-2-yl)ethyl)- 4-(isopropylsulfonyl)-1- ((phenoxathiine-3- carbonyl)glycyl)pyrrolidine-2- carboxamide |
| 134 | 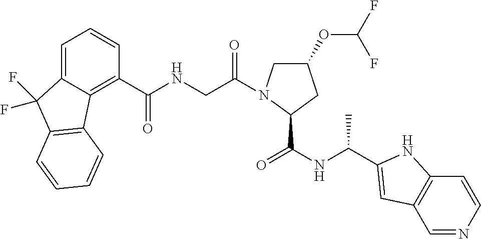 [SMILES: C[C@@H](NC(=O)[C@@H]1C[C@@H](OC(F)F)CN1C(=O)CNC(=O)c1cccc2c1-c1ccccc1C2(F)F)c1cc2cnccc2[nH]1] | (2S,4R)-N-((R)-1-(1H- pyrrolo[3,2-c]pyridin-2-yl)ethyl)- 1-((9,9-difluoro-9H-fluorene-4- carbony)glycyl)-4- (difluoromethoxy)pyrrolidine-2- carboxamide |
| 135 | 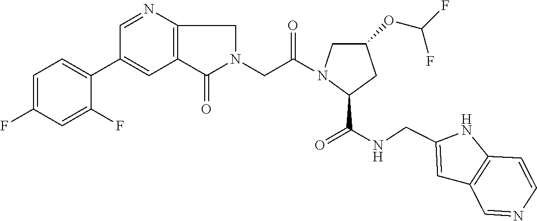 [SMILES: O=C(NCc1cc2cnccc2[nH]1)[C@@H]1C[C@@H](OC(F)F)CN1C(=O)CN1Cc2ncc(-c3ccc(F)cc3F)cc2C1=O] | (2S,4R)-N-((1H-pyrrolo[3,2- c]pyridin-2-yl)methyl)-4- (difluoromethoxy)-1-(2-(3-(2,4- difluorophenyl)-5-oxo-5,7- dihydro-6H-pyrrolo[3,4- b]pyridin-6-yl)acetyl)pyrrolidine- 2-carboxamide |
| 136 | 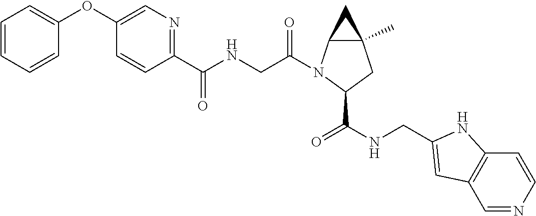 [SMILES: C[C@@]12C[C@@H]1N(C(=O)CNC(=O)c1ccc(Oc3ccccc3)cn1)[C@H](C(=O)NCc1cc3cnccc3[nH]1)C2] | (1S,3S,5S)-N-((1H-pyrrolo[3,2- c]pyridin-2-yl)methyl)-5-methyl- 2-((5-phenoxypicolinoyl)glycyl)- 2-azabicyclo[3.1.0]hexane-3- carboxamide |
| 137 | 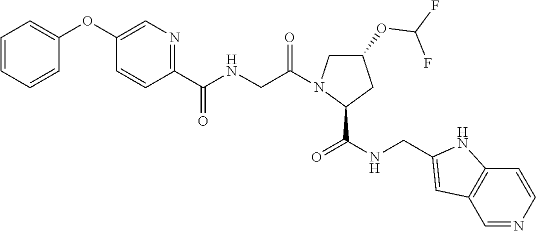 [SMILES: O=C(NCC(=O)N1C[C@H](OC(F)F)C[C@H]1C(=O)NCc1cc2cnccc2[nH]1)c1ccc(Oc2ccccc2)cn1] | N-(2-((2S,4R)-2-(((1H- pyrrolo[3,2-c]pyridin-2- yl)methyl)carbamoyl)-4- (difluoromethoxy)pyrrolidin-1- yl)-2-oxoethyl)-5- phenoxypicolinamide |
| 138 | 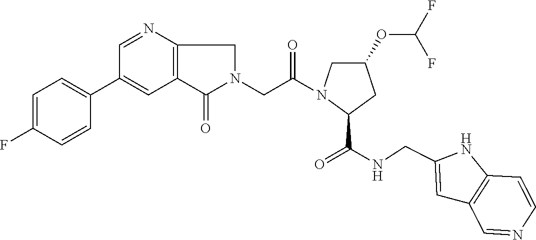 [SMILES: O=C(NCc1cc2cnccc2[nH]1)[C@@H]1C[C@@H](OC(F)F)CN1C(=O)CN1Cc2ncc(-c3ccc(F)cc3)cc2C1=O] | (2S,4R)-N-((1H-pyrrolo[3,2- c]pyridin-2-yl)methyl)-4- (difluoromethoxy)-1-(2-(3-(4- fluorophenyl)-5-oxo-5,7-dihydro- 6H-pyrrolo[3,4-b]pyridin-6- yl)acetyl)pyrrolidine-2- carboxamide |
| 139 | 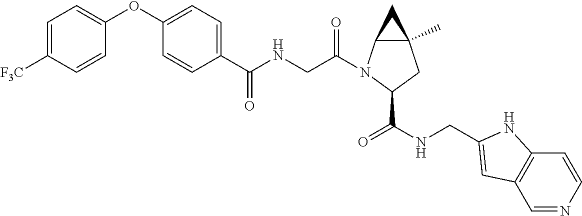 [SMILES: C[C@@]12C[C@@H]1N(C(=O)CNC(=O)c1ccc(Oc3ccc(C(F)(F)F)cc3)cc1)[C@H](C(=O)NCc1cc3cnccc3[nH]1)C2] | (1S,3S,5S)-N-((1H-pyrrolo[3,2- c]pyridin-2-yl)methyl)-5-methyl- 2-((4-(4- (trifluoromethyl)phenoxy) benzoyl)glycyl)-2- azabicyclo[3.1.0]hexane-3- carboxamide |
| 140 | 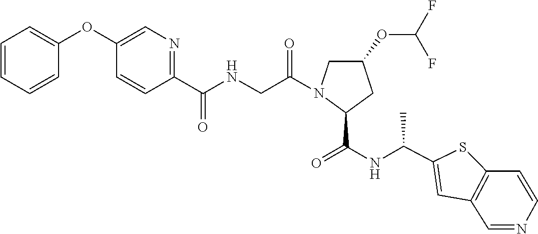 [SMILES: C[C@@H](NC(=O)[C@@H]1C[C@@H](OC(F)F)CN1C(=O)CNC(=O)c1ccc(Oc2ccccc2)cn1)c1cc2cnccc2s1] | N-(2-((2S,4R)-2-(((R)-1-(1H- pyrrolo[3,2-c]pyridin-2- yl)ethyl)carbamoyl)-4- (difluoromethoxy)pyrrolidin-1- yl)-2-oxoethyl)-5- phenoxypicolinamide |
| 141 | 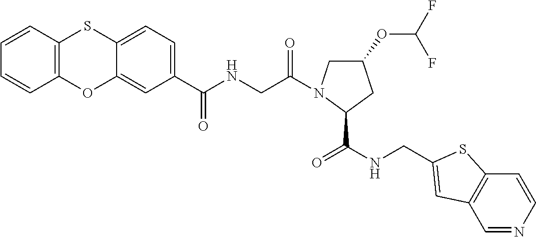 [SMILES: O=C(NCC(=O)N1C[C@H](OC(F)F)C[C@H]1C(=O)NCc1cc2cnccc2s1)c1ccc2c(c1)Oc1ccccc1S2] | (2S,4R)-4-(difluoromethoxy)-1- ((phenoxathiine-3- carbonyl)glycyl)-N-(thieno[3,2- c]pyridin-2-ylmethyl)pyrrolidine- 2-carboxamide |
| 142 | 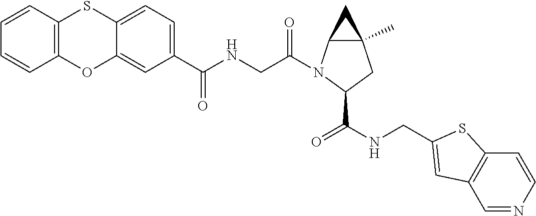 [SMILES: C[C@@]12C[C@@H]1N(C(=O)CNC(=O)c1ccc3c(c1)Oc1ccccc1S3)[C@H](C(=O)NCc1cc3cnccc3s1)C2] | (1S,3S,5S)-5-methyl-2- ((phenoxathiine-3- carbonyl)glycyl)-N-(thieno[3,2- c]pyridin-2-ylmethyl)-2- azabicyclo[3.1.0]hexane-3- carboxamide |
| 143 | 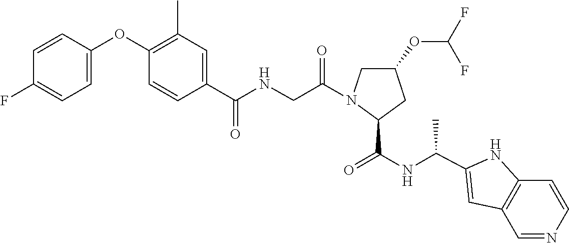 [SMILES: Cc1cc(C(=O)NCC(=O)N2C[C@H](OC(F)F)C[C@H]2C(=O)N[C@H](C)c2cc3cnccc3[nH]2)ccc1Oc1ccc(F)cc1] | (2S,4R)-N-((R)-1-(1H- pyrrolo[3,2-c]pyridin-2-yl)ethyl)- 4-(difluoromethoxy)-1-((4-(4- fluorophenoxy)-3- methylbenzoyl)glycyl)pyrrolidine- 2-carboxamide |
| 144 | 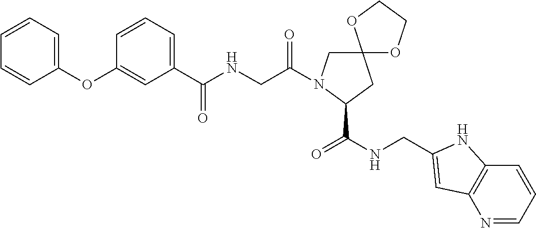 [SMILES: O=C(NCC(=O)N1CC2(C[C@H]1C(=O)NCc1cc3ncccc3[nH]1)OCCO2)c1cccc(Oc2ccccc2)c1] | (S)-N-((1H-pyrrolo[3,2-c]pyridin- 2-yl)methyl)-7-((3- phenoxybenzoyl)glycyl)-1,4- dioxa-7-azaspiro[4.4]nonane-8- carboxamide |
| 145 | 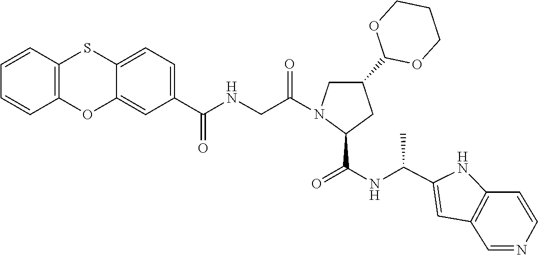 [SMILES: C[C@@H](NC(=O)[C@@H]1C[C@@H](C2OCCCO2)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 4-(1,3-dioxan-2-yl)-1- ((phenoxathiine-3- carbonyl)glycyl)pyrrolidine-2- carboxamide |
| 146 | 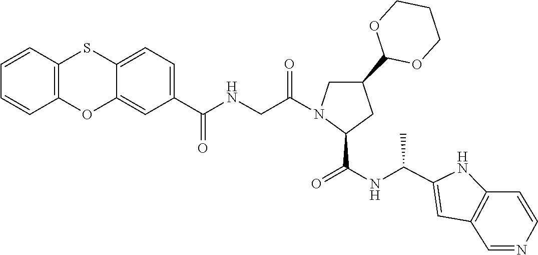 [SMILES: C[C@@H](NC(=O)[C@@H]1C[C@H](C2OCCCO2)CN1C(=O)CNC(=O)c1ccc2c(c1)Oc1ccccc1S2)c1cc2cnccc2[nH]1] | (2S,4S)-N-((R)-1-(1H- pyrrolo[3,2-c]pyridin-2-yl)ethyl)- 4-(1,3-dioxan-2-yl)-1- ((phenoxathiine-3- carbonyl)glycyl)pyrrolidine-2- carboxamide |
| 147 | 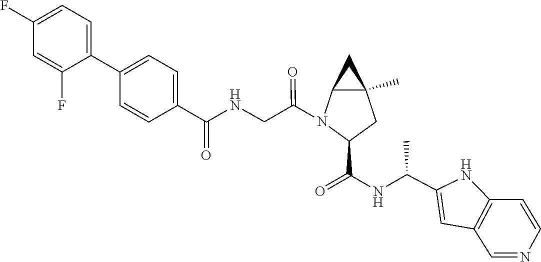 [SMILES: C[C@@H](NC(=O)[C@@H]1C[C@]2(C)C[C@@H]2N1C(=O)CNC(=O)c1ccc(-c2ccc(F)cc2F)cc1)c1cc2cnccc2[nH]1] | (1S,3S,5S)-N-((R)-1-(1H- pyrrolo[3,2-c]pyridin-2-yl)ethyl)- 2-((2′,4′-difluoro-[1,1′-biphenyl]- 4-carbonyl)glycyl)-5-methyl-2- azabicyclo[3.1.0]hexane-3- carboxamide |
| 148 | 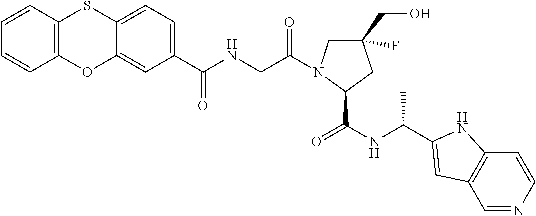 [SMILES: C[C@@H](NC(=O)[C@@H]1C[C@](F)(CO)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 4-fluoro-4-(hydroxymethyl)-1- ((phenoxathiine-3- carbonyl)glycyl)pyrrolidine-2- carboxamide |
| 149 | 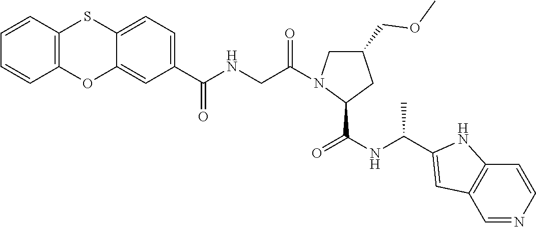 [SMILES: COC[C@@H]1C[C@@H](C(=O)N[C@H](C)c2cc3cnccc3[nH]2)N(C(=O)CNC(=O)c2ccc3c(c2)Oc2ccccc2S3)C1] | (2S,4R)-N-((R)-1-(1H- pyrrolo[3,2-c]pyridin-2-yl)ethyl)- 4-(methoxymethyl)-1- ((phenoxathiine-3- carbonyl)glycyl)pyrrolidine-2- carboxamide |
| 150 | 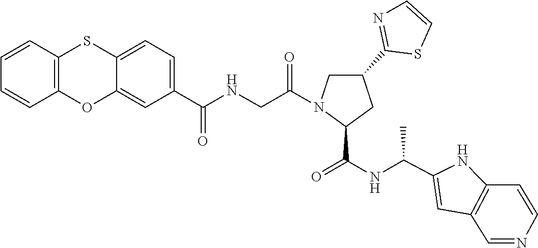 [SMILES: C[C@@H](NC(=O)[C@@H]1C[C@@H](c2nccs2)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 1-((phenoxathiine-3- carbonyl)glycyl)-4-(thiazol-2- yl)pyrrolidine-2-carboxamide |
| 151 | 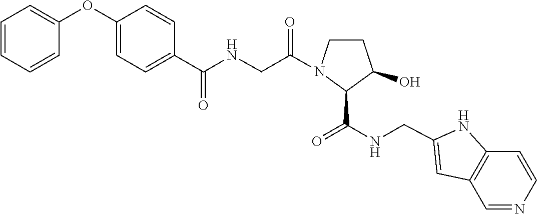 [SMILES: O=C(NCC(=O)N1CC[C@@H](O)[C@H]1C(=O)NCc1cc2cnccc2[nH]1)c1ccc(Oc2ccccc2)cc1] | (2S,3R)-N-((1H-pyrrolo[3,2- c]pyridin-2-yl)methyl)-3- hydroxy-1-((4- phenoxybenzoyl)glycyl) pyrrolidine-2-carboxamide |
| 152 | 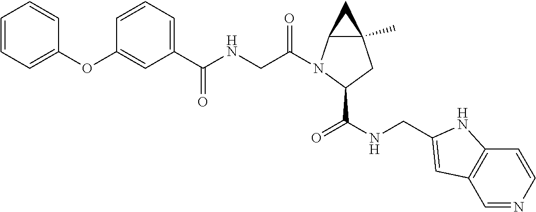 [SMILES: C[C@@]12C[C@@H]1N(C(=O)CNC(=O)c1cccc(Oc3ccccc3)c1)[C@H](C(=O)NCc1cc3cnccc3[nH]1)C2] | (1S,3S,5S)-N-((1H-pyrrolo[3,2- c]pyridin-2-yl)methyl)-5-methyl- 2-((3-phenoxybenzoyl)glycyl)-2- azabicyclo[3.1.0]hexane-3- carboxamide |
| 153 | 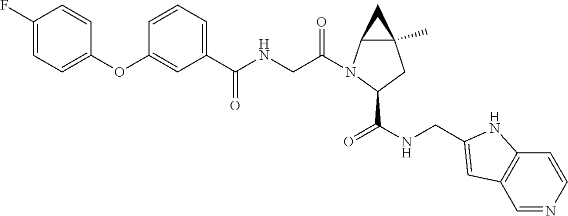 [SMILES: C[C@@]12C[C@@H]1N(C(=O)CNC(=O)c1cccc(Oc3ccc(F)cc3)c1)[C@H](C(=O)NCc1cc3cnccc3[nH]1)C2] | (1S,3S,5S)-N-((1H-pyrrolo[3,2- c]pyridin-2-yl)methyl)-2-((3-(4- fluorophenoxy)benzoyl)glycyl)-5- methyl-2- azabicyclo[3.1.0]hexane-3- carboxamide |
| 154 | 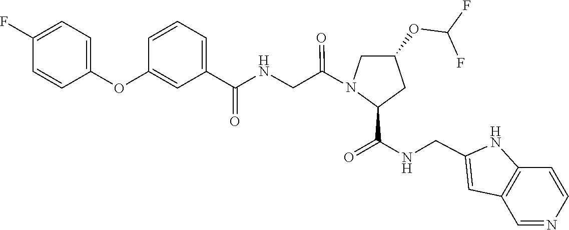 [SMILES: O=C(NCC(=O)N1C[C@H](OC(F)F)C[C@H]1C(=O)NCc1cc2cnccc2[nH]1)c1cccc(Oc2ccc(F)cc2)c1] | (2S,4R)-N-((1H-pyrrolo[3,2- c]pyridin-2-yl)methyl)-4- (difluoromethoxy)-1-((3-(4- fluorophenoxy)benzoyl)glycyl) pyrrolidine-2-carboxamide |
| 155 | 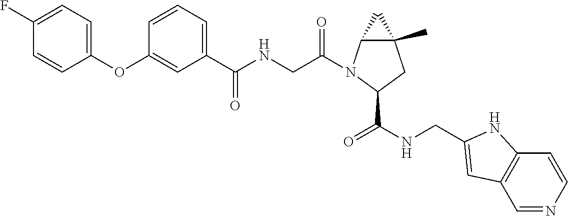 [SMILES: C[C@@]12C[C@@H](C(=O)NCc3cc4cnccc4[nH]3)N(C(=O)CNC(=O)c3cccc(Oc4ccc(F)cc4)c3)[C@@H]1C2] | (1R,3S,5R)-N-((1H-pyrrolo[3,2- c]pyridin-2-yl)methyl)-2-((3-(4- fluorophenoxy)benzoyl)glycyl)-5- methyl-2- azabicyclo[3.1.0]hexane-3- carboxamide |
| 156 | 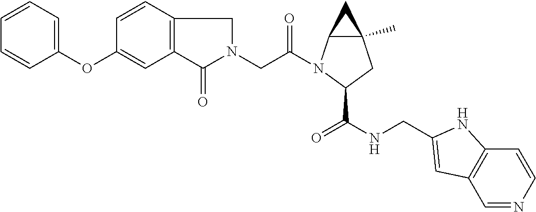 [SMILES: C[C@@]12C[C@@H]1N(C(=O)CN1Cc3ccc(Oc4ccccc4)cc3C1=O)[C@H](C(=O)NCc1cc3cnccc3[nH]1)C2] | (1S,3S,5S)-N-((1H-pyrrolo[3,2- c]pyridin-2-yl)methyl)-5-methyl- 2-(2-(1-oxo-6-phenoxyisoindolin- 2-yl)acetyl)-2- azabicyclo[3.1.0]hexane-3- carboxamide |
| 157 | 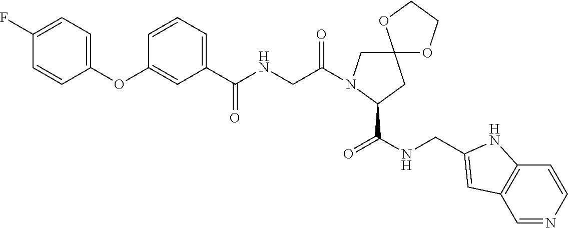 [SMILES: O=C(NCC(=O)N1CC2(C[C@H]1C(=O)NCc1cc3cnccc3[nH]1)OCCO2)c1cccc(Oc2ccc(F)cc2)c1] | (S)-N-((1H-pyrrolo[3,2-c]pyridin- 2-yl)methyl)-7-((3-(4- fluorophenoxy)benzoyl)glycyl)- 1,4-dioxa-7-azaspiro[4.4]nonane- 8-carboxamide |
| 158 | 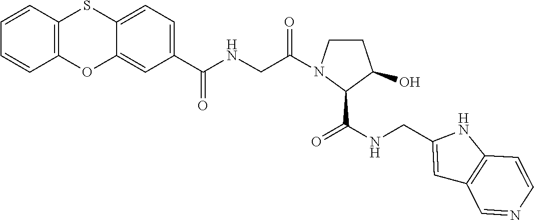 [SMILES: O=C(NCC(=O)N1CC[C@@H](O)[C@H]1C(=O)NCc1cc2cnccc2[nH]1)c1ccc2c(c1)Oc1ccccc1S2] | (2S,3R)-N-((1H-pyrrolo[3,2- c]pyridin-2-yl)methyl)-3- hydroxy-1-((phenoxathiine-3- carbonyl)glycyl)pyrrolidine-2- carboxamide |
| 159 | 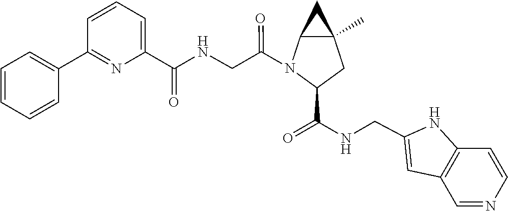 [SMILES: C[C@@]12C[C@@H]1N(C(=O)CNC(=O)c1cccc(-c3ccccc3)n1)[C@H](C(=O)NCc1cc3cnccc3[nH]1)C2] | (1S,3S,5S)-N-((1H-pyrrolo[3,2- c]pyridin-2-yl)methyl)-5-methyl- 2-((6-phenylpicolinoyl)glycyl)-2- azabicyclo[3.1.0]hexane-3- carboxamide |
| 160 | 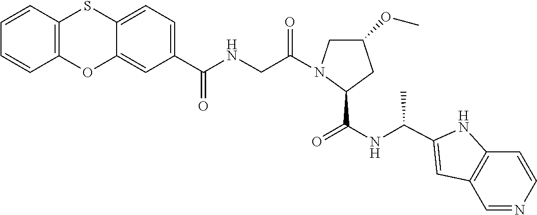 [SMILES: CO[C@@H]1C[C@@H](C(=O)N[C@H](C)c2cc3cnccc3[nH]2)N(C(=O)CNC(=O)c2ccc3c(c2)Oc2ccccc2S3)C1] | (2S,4R)-N-((R)-1-(1H- pyrrolo[3,2-c]pyridin-2-yl)ethyl)- 4-methoxy-1-((phenoxathiine-3- carbonyl)glycyl)pyrrolidine-2- carboxamide |
| 161 | 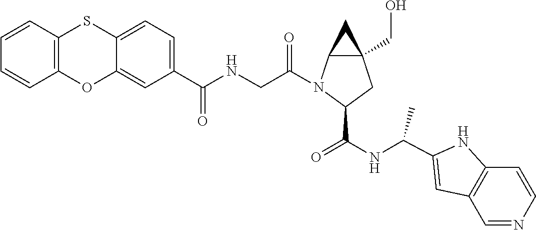 [SMILES: C[C@@H](NC(=O)[C@@H]1C[C@]2(CO)C[C@@H]2N1C(=O)CNC(=O)c1ccc2c(c1)Oc1ccccc1S2)c1cc2cnccc2[nH]1] | (1S,3S,5R)-N-((R)-1-(1H- pyrrolo[3,2-c]pyridin-2-yl)ethyl)- 5-(hydroxymethyl)-2- ((phenoxathiine-3- carbonyl)glycyl)-2- azabicyclo[3.1.0]hexane-3- carboxamide |
| 162 | 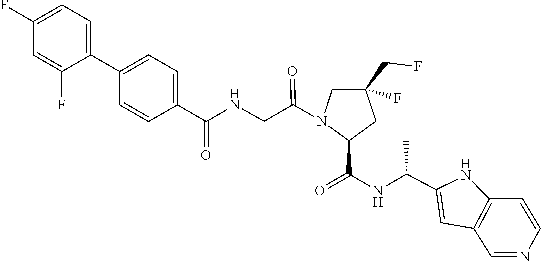 [SMILES: C[C@@H](NC(=O)[C@@H]1C[C@](F)(CF)CN1C(=O)CNC(=O)c1ccc(-c2ccc(F)cc2F)cc1)c1cc2cnccc2[nH]1] | (2S,4R)-N-((R)-1-(1H- pyrrolo[3,2-c]pyridin-2-yl)ethyl)- 1-((2′,4′-difluoro-[1,1′-biphenyl]- 4-carbonyl)glycyl)-4-fluoro-4- (fluoromethyl)pyrrolidine-2- carboxamide |
| 163 | 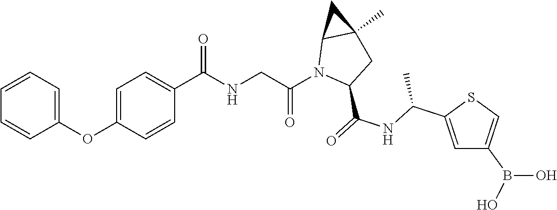 [SMILES: C[C@@H](NC(=O)[C@@H]1C[C@]2(C)C[C@@H]2N1C(=O)CNC(=O)c1ccc(Oc2ccccc2)cc1)c1cc(B(O)O)cs1] | (5-((R)-1-((1S,3S,5S)-5-methyl-2- ((4-phenoxybenzoyl)glycyl)-2- azabicyclo[3.1.0]hexane-3- carboxamido)ethyl)thiophen-3- yl)boronic acid |
| 164 | 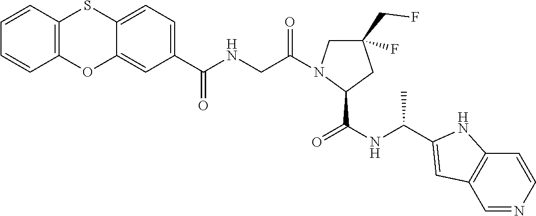 [SMILES: C[C@@H](NC(=O)[C@@H]1C[C@](F)(CF)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 4-fluoro-4-(fluoromethyl)-1- ((phenoxathiine-3- carbonyl)glycyl)pyrrolidine-2- carboxamide |
| 165 | 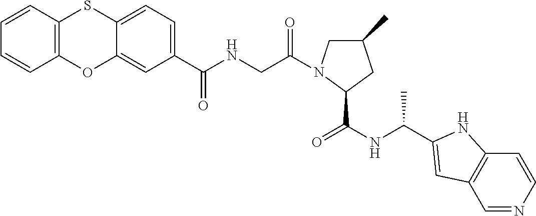 [SMILES: C[C@H]1C[C@@H](C(=O)N[C@H](C)c2cc3cnccc3[nH]2)N(C(=O)CNC(=O)c2ccc3c(c2)Oc2ccccc2S3)C1] | (2S,4S)-N-((R)-1-(1H- pyrrolo[3,2-c]pyridin-2-yl)ethyl)- 4-methyl-1-((phenoxathiine-3- carbonyl)glycyl)pyrrolidine-2- carboxamide |
| 166 | 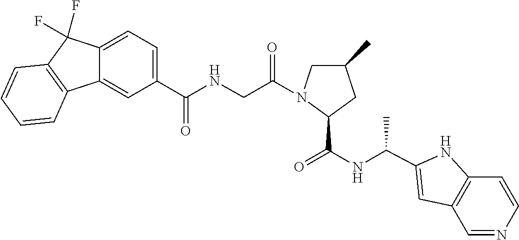 [SMILES: C[C@H]1C[C@@H](C(=O)N[C@H](C)c2cc3cnccc3[nH]2)N(C(=O)CNC(=O)c2ccc3c(c2)-c2ccccc2C3(F)F)C1] | (2S,4S)-N-((R)-1-(1H- pyrrolo[3,2-c]pyridin-2-yl)ethyl)- 1-((9,9-difluoro-9H-fluorene-3- carbonyl)glycyl)-4- methylpyrrolidine-2-carboxamide |
| 167 | 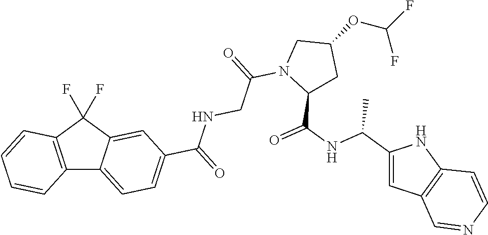 [SMILES: C[C@@H](NC(=O)[C@@H]1C[C@@H](OC(F)F)CN1C(=O)CNC(=O)c1ccc2c(c1)C(F)(F)c1ccccc1-2)c1cc2cnccc2[nH]1] | (2S,4R)-N-((R)-1-(1H- pyrrolo[3,2-c]pyridin-2-yl)ethyl)- 1-((9,9-difluoro-9H-fluorene-2- carbonyl)glycyl)-4- (difluoromethoxy)pyrrolidine-2- carboxamide |
| 168 | 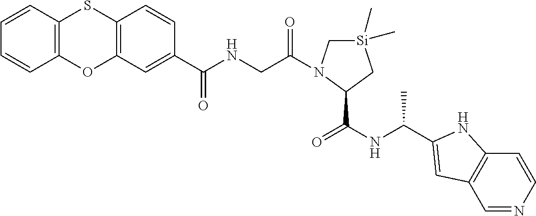 [SMILES: C[C@@H](NC(=O)[C@@H]1C[Si](C)(C)CN1C(=O)CNC(=O)c1ccc2c(c1)Oc1ccccc1S2)c1cc2cnccc2[nH]1] | (R)-N-((R)-1-(1H-pyrrolo[3,2- c]pyridin-2-yl)ethyl)-3,3- dimethyl-1-((phenoxathiine-3- carbonyl)glycyl)-1,3- azasilolidine-5-carboxamide |
| 169 | 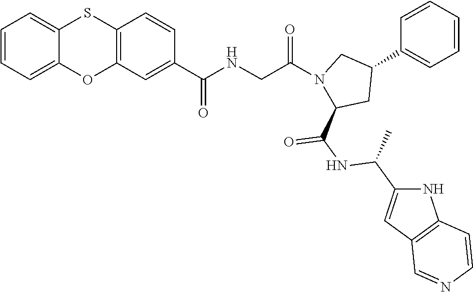 [SMILES: C[C@@H](NC(=O)[C@@H]1C[C@@H](c2ccccc2)CN1C(=O)CNC(=O)c1ccc2c(c1)Oc1ccccc1S2)c1cc2cnccc2[nH]1] | (2S,4S)-N-((R)-1-(1H- pyrrolo[3,2-c]pyridin-2-yl)ethyl)- 1-((phenoxathiine-3- carbonyl)glycyl)-4- phenylpyrrolidine-2-carboxamide |
| 170 | 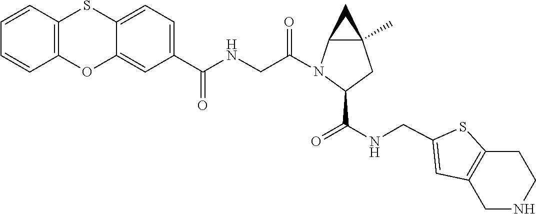 [SMILES: C[C@@]12C[C@@H]1N(C(=O)CNC(=O)c1ccc3c(c1)Oc1ccccc1S3)[C@H](C(=O)NCc1cc3c(s1)CCNC3)C2] | (1S,3S,5S)-5-methyl-2- ((phenoxathiine-3- carbonyl)glycyl)-N-((4,5,6,7- tetrahydrothieno[3,2-c]pyridin-2- yl)methyl)-2- azabicyclo[3.1.0]hexane-3- carboxamide |
| 171 | 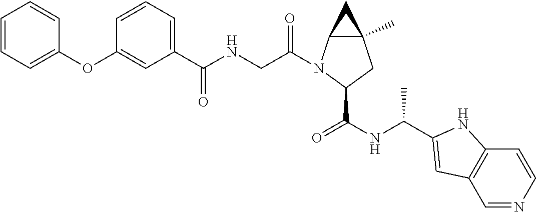 [SMILES: C[C@@H](NC(=O)[C@@H]1C[C@]2(C)C[C@@H]2N1C(=O)CNC(=O)c1cccc(Oc2ccccc2)c1)c1cc2cnccc2[nH]1] | (1S,3S,5S)-N-((R)-1-(1H- pyrrolo[3,2-c]pyridin-2-yl)ethyl)- 5-methyl-2-((3- phenoxybenzoyl)glycyl)-2- azabicyclo[3.1.0]hexane-3- carboxamide |
| 172 | 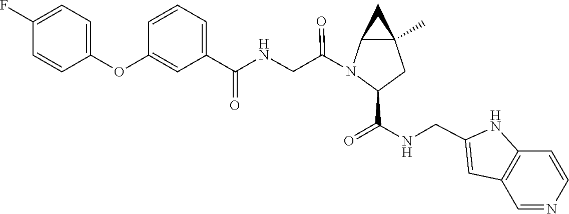 [SMILES: C[C@@]12C[C@@H]1N(C(=O)CNC(=O)c1cccc(Oc3ccc(F)cc3)c1)[C@H](C(=O)NCc1cc3cnccc3[nH]1)C2] | (1S,3S,5S)-N-((R)-1-(1H- pyrrolo[3,2-c]pyridin-2-yl)ethyl)- 2-((3-(4- fluorophenoxy)benzoyl)glycyl)-5- methyl-2- azabicyclo[3.1.0]hexane-3- carboxamide |
| 173 | 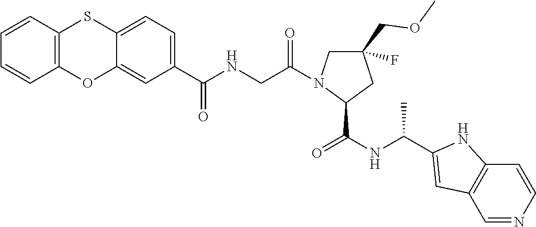 [SMILES: COC[C@@]1(F)C[C@@H](C(=O)N[C@H](C)c2cc3cnccc3[nH]2)N(C(=O)CNC(=O)c2ccc3c(c2)Oc2ccccc2S3)C1] | (2S,4R)-N-((R)-1-(1H- pyrrolo[3,2-c]pyridin-2-yl)ethyl)- 4-fluoro-4-(methoxymethyl)-1- ((phenoxathiine-3- carbonyl)glycyl)pyrrolidine-2- carboxamide |
| 174 | 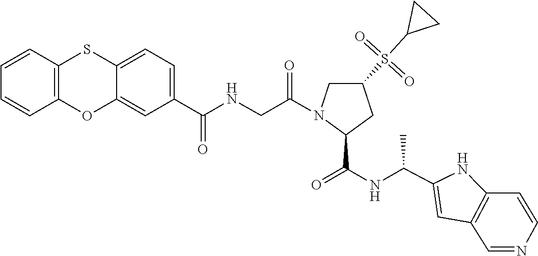 [SMILES: C[C@@H](NC(=O)[C@@H]1C[C@@H](S(=O)(=O)C2CC2)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 4-(cyclopropylsulfonyl)-1- ((phenoxathiine-3- carbonyl)glycyl)pyrrolidine-2- carboxamide |
| 175 | 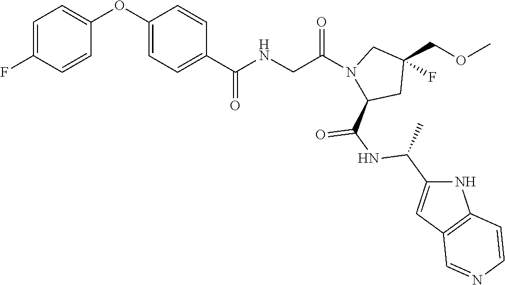 [SMILES: COC[C@@]1(F)C[C@@H](C(=O)N[C@H](C)c2cc3cnccc3[nH]2)N(C(=O)CNC(=O)c2ccc(Oc3ccc(F)cc3)cc2)C1] | (2S,4R)-N-((R)-1-(1H- pyrrolo[3,2-c]pyridin-2-yl)ethyl)- 4-fluoro-1-((4-(4- fluorophenoxy)benzoyl)glycyl)-4- (methoxymethyl)pyrrolidine-2- carboxamide |
| 176 | 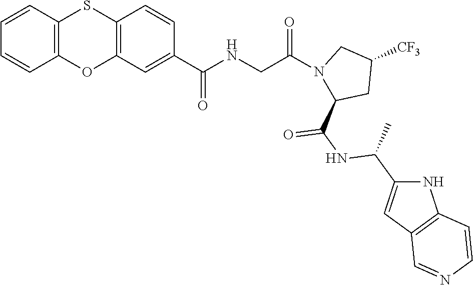 [SMILES: C[C@@H](NC(=O)[C@@H]1C[C@@H](C(F)(F)F)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 1-((phenoxathiine-3- carbonyl)glycyl)-4- (trifluoromethyl)pyrrolidine-2- carboxamide |
| 177 | 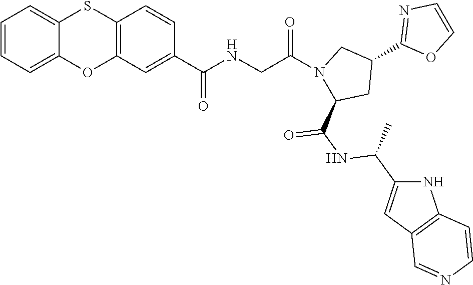 [SMILES: C[C@@H](NC(=O)[C@@H]1C[C@@H](c2ncco2)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 4-(oxazol-2-yl)-1- ((phenoxathiine-3- carbonyl)glycyl)pyrrolidine-2- carboxamide |
| 178 | 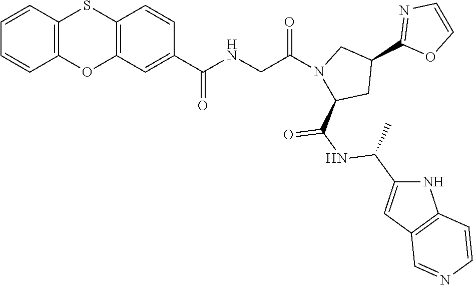 [SMILES: C[C@@H](NC(=O)[C@@H]1C[C@H](c2ncco2)CN1C(=O)CNC(=O)c1ccc2c(c1)Oc1ccccc1S2)c1cc2cnccc2[nH]1] | (2S,4S)-N-((R)-1-(1H- pyrrolo[3,2-c]pyridin-2-yl)ethyl)- 4-(oxazol-2-yl)-1- ((phenoxathiine-3- carbonyl)glycyl)pyrrolidine-2- carboxamide |
| 179 | 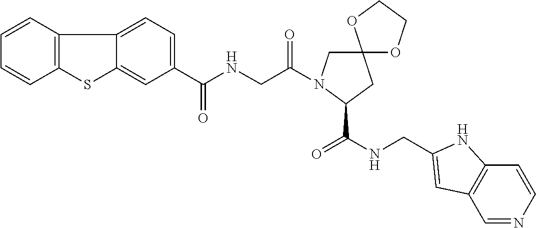 [SMILES: O=C(NCC(=O)N1CC2(C[C@H]1C(=O)NCc1cc3cnccc3[nH]1)OCCO2)c1ccc2c(c1)sc1ccccc12] | (S)-N-((1H-pyrrolo[3,2-c]pyridin- 2-yl)methyl)-7- ((dibenzo[b,d]thiophene-3- carbonyl)glycyl)-1,4-dioxa-7- azaspiro[4.4]nonane-8- carboxamide |
| 180 | 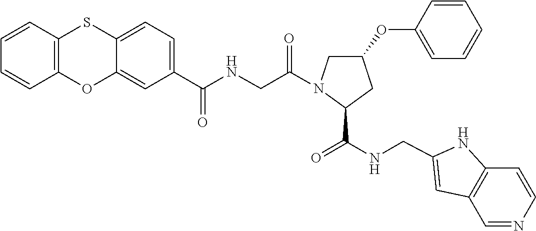 [SMILES: O=C(NCC(=O)N1C[C@H](Oc2ccccc2)C[C@H]1C(=O)NCc1cc2cnccc2[nH]1)c1ccc2c(c1)Oc1ccccc1S2] | (2S,4R)-N-((R)-1-(1H- pyrrolo[3,2-c]pyridin-2-yl)ethyl)- 1-((phenoxathiine-3- carbonyl)glycyl)-4- phenoxypyrrolidine-2- carboxamide |
| 181 | 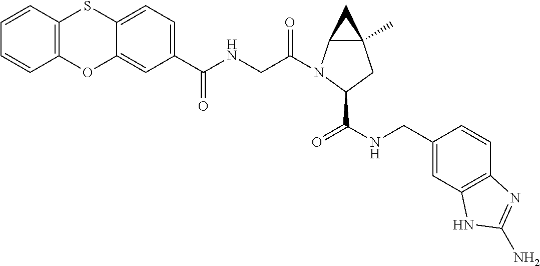 [SMILES: C[C@@]12C[C@@H]1N(C(=O)CNC(=O)c1ccc3c(c1)Oc1ccccc1S3)[C@H](C(=O)NCc1ccc3nc(N)[nH]c3c1)C2] | (1S,3S,5S)-N-((2-amino-1H- benzo[d]imidazol-6-yl)methyl)-5- methyl-2-((phenoxathiine-3- carbonyl)glycyl)-2- azabicyclo[3.1.0]hexane-3- carboxamide |
| 182 | 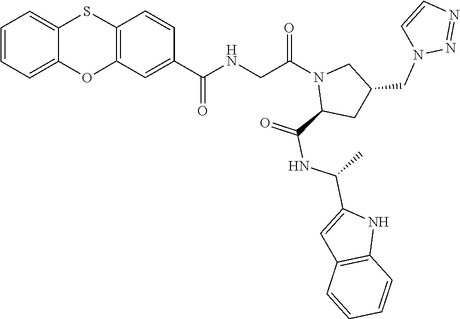 [SMILES: C[C@@H](NC(=O)[C@@H]1C[C@@H](Cn2ccnn2)CN1C(=O)CNC(=O)c1ccc2c(c1)Oc1ccccc1S2)c1cc2ccccc2[nH]1] | (2S,4R)-4-((1H-1,2,3-triazol-1- yl)methyl)-N-((R)-1-(1H- pyrrolo[3,2-c]pyridin-2-yl)ethyl)- 1-((phenoxathiine-3- carbonyl)glycyl)pyrrolidine-2- carboxamide |
| 183 | 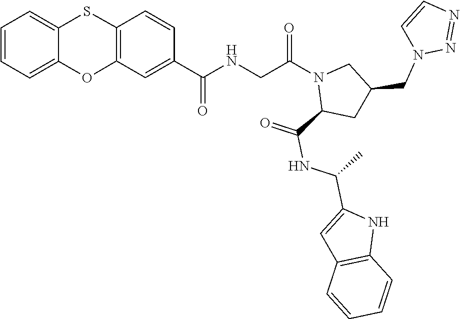 [SMILES: C[C@@H](NC(=O)[C@@H]1C[C@H](Cn2ccnn2)CN1C(=O)CNC(=O)c1ccc2c(c1)Oc1ccccc1S2)c1cc2ccccc2[nH]1] | (2S,4S)-4-((1H-1,2,3-triazol-1- yl)methyl)-N-((R)-1-(1H- pyrrolo[3,2-c]pyridin-2-yl)ethyl)- 1-((phenoxathiine-3- carbonyl)glycyl)pyrrolidine-2- carboxamide |
| 184 | 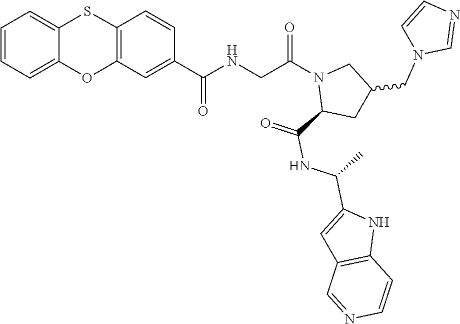 [SMILES: C[C@@H](NC(=O)[C@@H]1CC(Cn2ccnc2)CN1C(=O)CNC(=O)c1ccc2c(c1)Oc1ccccc1S2)c1cc2cnccc2[nH]1] | (2S)-4-((1H-imidazol-1- yl)methyl)-N-((R)-1-(1H- pyrrolo[3,2-c]pyridin-2-yl)ethyl)- 1-((phenoxathiine-3- carbonyl)glycyl)pyrrolidine-2- carboxamide |
| 185 | 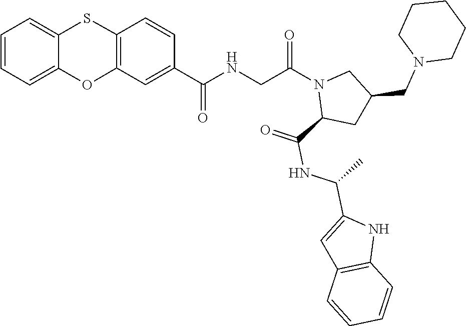 [SMILES: C[C@@H](NC(=O)[C@@H]1C[C@H](CN2CCCCC2)CN1C(=O)CNC(=O)c1ccc2c(c1)Oc1ccccc1S2)c1cc2ccccc2[nH]1] | (2S)-N-((R)-1-(1H-pyrrolo[3,2- c]pyridin-2-yl)ethyl)-1- ((phenoxathiine-3- carbonyl)glycyl)-4-(piperidin-1- ylmethyl)pyrrolidine-2- carboxamide |
| 186 | 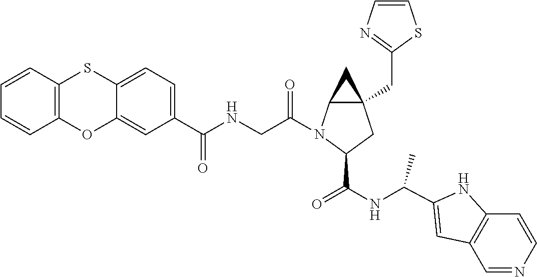 [SMILES: C[C@@H](NC(=O)[C@@H]1C[C@]2(Cc3nccs3)C[C@@H]2N1C(=O)CNC(=O)c1ccc2c(c1)Oc1ccccc1S2)c1cc2cnccc2[nH]1] | (1S,3S,5S)-N-((R)-1-(1H- pyrrolo[3,2-c]pyridin-2-yl)ethyl)- 2-((phenoxathiine-3- carbonyl)glycyl)-5-(thiazol-2- ylmethyl)-2- azabicyclo[3.1.0]hexane-3- carboxamide |
| 187 | 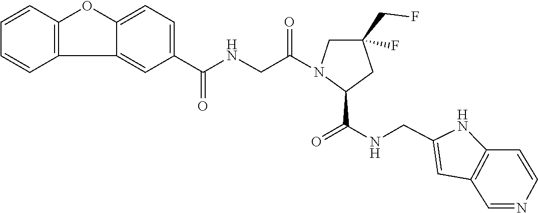 [SMILES: O=C(NCC(=O)N1C[C@@](F)(CF)C[C@H]1C(=O)NCc1cc2cnccc2[nH]1)c1ccc2oc3ccccc3c2c1] | (2S,4R)-N-((1H-pyrrolo[3,2- c]pyridin-2-yl)methyl)-1- ((dibenzo[b,d]furan-2- carbonyl)glycyl)-4-fluoro-4- (fluoromethyl)pyrrolidine-2- carboxamide |
| 188 | 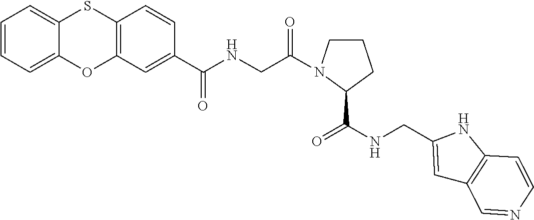 [SMILES: O=C(NCC(=O)N1CCC[C@H]1C(=O)NCc1cc2cnccc2[nH]1)c1ccc2c(c1)Oc1ccccc1S2] | (S)-N-((1H-pyrrolo[3,2-c]pyridin- 2-yl)methyl)-1-((phenoxathiine-3- carbonyl)glycyl)pyrrolidine-2- carboxamide |
| 189 | 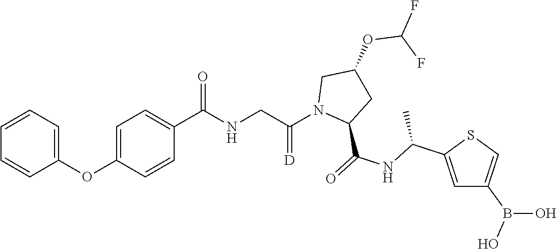 | (5-((R)-1-((2S,4R)-4- (difluoromethoxy)-1-((4- phenoxybenzoyl)glycyl)pyrrolidine- 2-carboxamido)ethyl)thiophen- 3-yl)boronic acid |
| 190 | 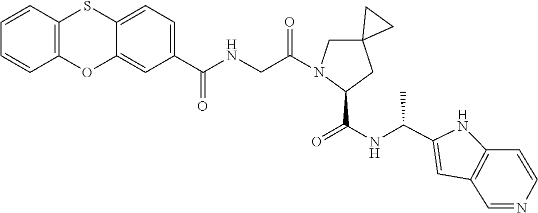 [SMILES: C[C@@H](NC(=O)[C@@H]1CC2(CC2)CN1C(=O)CNC(=O)c1ccc2c(c1)Oc1ccccc1S2)c1cc2cnccc2[nH]1] | (S)-N-((R)-1-(1H-pyrrolo[3,2- c]pyridin-2-yl)ethyl)-5- ((phenoxathiine-3- carbonyl)glycyl)-5- azaspiro[2.4]heptane-6- carboxamide |
| 191 | 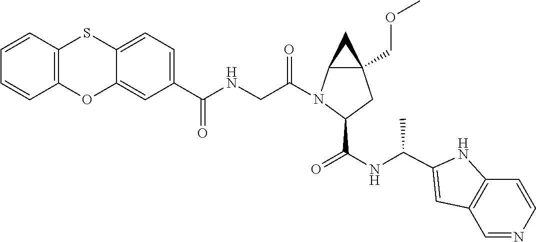 [SMILES: COC[C@@]12C[C@@H]1N(C(=O)CNC(=O)c1ccc3c(c1)Oc1ccccc1S3)[C@H](C(=O)N[C@H](C)c1cc3cnccc3[nH]1)C2] | (1S,3S,5R)-N-((R)-1-(1H- pyrrolo[3,2-c]pyridin-2-yl)ethyl)- 5-(methoxymethyl)-2- ((phenoxathiine-3- carbonyl)glycyl)-2- azabicyclo[3.1.0]hexane-3- carboxamide |
| 192 | 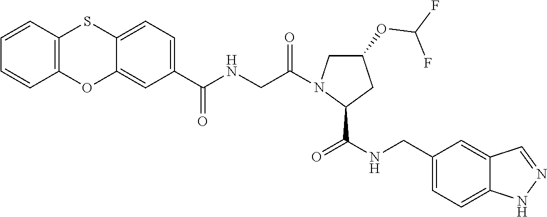 [SMILES: O=C(NCC(=O)N1C[C@H](OC(F)F)C[C@H]1C(=O)NCc1ccc2[nH]ncc2c1)c1ccc2c(c1)Oc1ccccc1S2] | (2S,4R)-N-((1H-indazol-5- yl)methyl)-4-(difluoromethoxy)- 1-((phenoxathiine-3- carbonyl)glycyl)pyrrolidine-2- carboxamide |
| 193 | 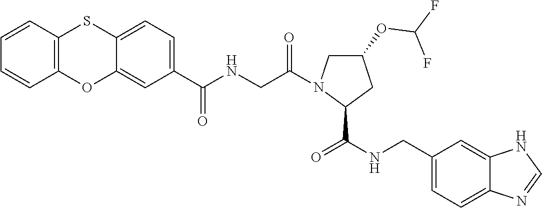 [SMILES: O=C(NCC(=O)N1C[C@H](OC(F)F)C[C@H]1C(=O)NCc1ccc2nc[nH]c2c1)c1ccc2c(c1)Oc1ccccc1S2] | (2S,4R)-N-((1H- benzo[d]imidazol-6-yl)methyl)-4- (difluoromethoxy)-1- ((phenoxathiine-3- carbonyl)glycyl)pyrrolidine-2- carboxamide |
| 194 | 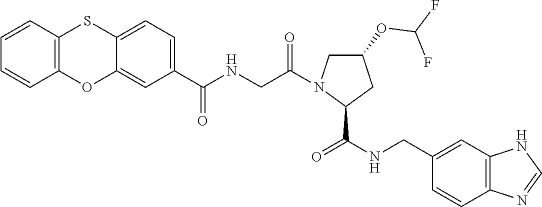 [SMILES: O=C(NCC(=O)N1C[C@H](OC(F)F)C[C@H]1C(=O)NCc1ccc2nc[nH]c2c1)c1ccc2c(c1)Oc1ccccc1S2] | (2S,4R)-N-((1H-pyrrolo[3,2- b]pyridin-6-yl)methyl)-4- (difluoromethoxy)-1- ((phenoxathiine-3- carbonyl)glycyl)pyrrolidine-2- carboxamide |
| 195 | 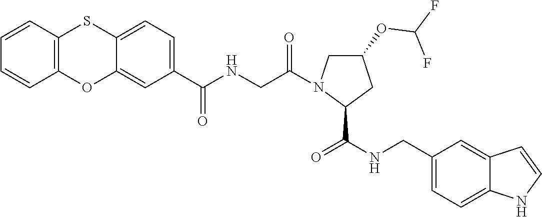 [SMILES: O=C(NCC(=O)N1C[C@H](OC(F)F)C[C@H]1C(=O)NCc1ccc2[nH]ccc2c1)c1ccc2c(c1)Oc1ccccc1S2] | (2S,4R)-N-((1H-indol-5- yl)methyl)-4-(difluoromethoxy)- 1-((phenoxathiine-3- carbonyl)glycyl)pyrrolidine-2- carboxamide |
| 196 | 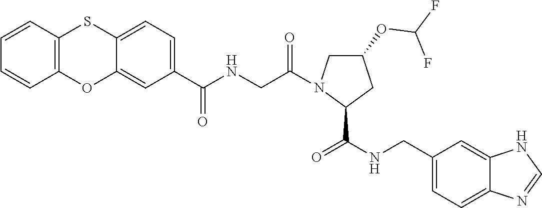 [SMILES: O=C(NCC(=O)N1C[C@H](OC(F)F)C[C@H]1C(=O)NCc1ccc2nc[nH]c2c1)c1ccc2c(c1)Oc1ccccc1S2] | (2S,4R)-N-((1H-indol-6- yl)methyl)-4-(difluoromethoxy)- 1-((phenoxathiine-3- carbonyl)glycyl)pyrrolidine-2- carboxamide |
| 197 | 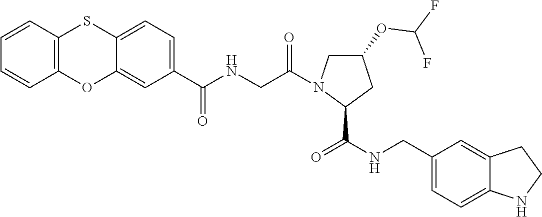 [SMILES: O=C(NCC(=O)N1C[C@H](OC(F)F)C[C@H]1C(=O)NCc1ccc2c(c1)CCN2)c1ccc2c(c1)Oc1ccccc1S2] | (2S,4R)-4-(difluoromethoxy)-N- (indolin-5-ylmethyl)-1- ((phenoxathiine-3- carbonyl)glycyl)pyrrolidine-2- carboxamide |
| 198 | 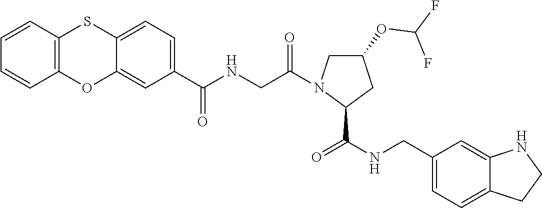 [SMILES: O=C(NCC(=O)N1C[C@H](OC(F)F)C[C@H]1C(=O)NCc1ccc2c(c1)NCC2)c1ccc2c(c1)Oc1ccccc1S2] | (2S,4R)-4-(difluoromethoxy)- N-(indolin-6-ylmethyl)-1- ((phenoxathiine-3- carbonyl)glycyl)pyrrolidine-2- carboxamide |
| 199 | 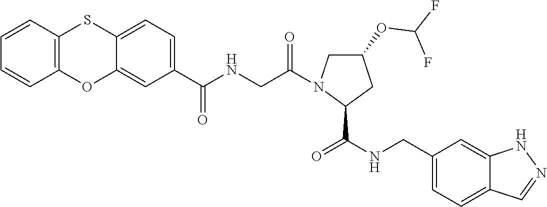 [SMILES: O=C(NCC(=O)N1C[C@H](OC(F)F)C[C@H]1C(=O)NCc1ccc2cn[nH]c2c1)c1ccc2c(c1)Oc1ccccc1S2] | (2S,4R)-N-((1H-indazol-6- yl)methyl)-4-(difluoromethoxy)- 1-((phenoxathiine-3- carbonyl)glycyl)pyrrolidine-2- carboxamide |
| 200 | 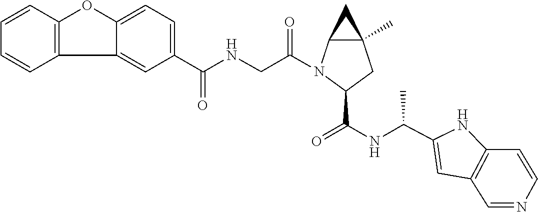 [SMILES: C[C@@H](NC(=O)[C@@H]1C[C@]2(C)C[C@@H]2N1C(=O)CNC(=O)c1ccc2oc3ccccc3c2c1)c1cc2cnccc2[nH]1] | (1S,3S,5S)-N-((R)-1-(1H- pyrrolo[3,2-c]pyridin-2-yl)ethyl)- 2-((dibenzo[b,d]furan-2- carbonyl)glycyl)-5-methyl-2- azabicyclo[3.1.0]hexane-3- carboxamide |
| 201 | 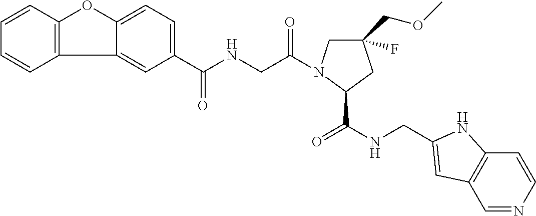 [SMILES: COC[C@@]1(F)C[C@@H](C(=O)NCc2cc3cnccc3[nH]2)N(C(=O)CNC(=O)c2ccc3oc4ccccc4c3c2)C1] | (2S,4R)-N-((1H-pyrrolo[3,2- c]pyridin-2-yl)methyl)-1- ((dibenzo[b,d]furan-2- carbonyl)glycyl)-4-fluoro-4- (methoxymethyl)pyrrolidine-2- carboxamide |
| 202 | 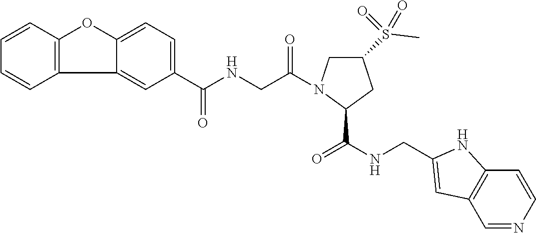 [SMILES: CS(=O)(=O)[C@@H]1C[C@@H](C(=O)NCc2cc3cnccc3[nH]2)N(C(=O)CNC(=O)c2ccc3oc4ccccc4c3c2)C1] | (2S,4R)-N-((1H-pyrrolo[3,2- c]pyridin-2-yl)methyl)-1- ((dibenzo[b,d]furan-2- carbonyl)glycyl)-4- (methylsulfonyl)pyrrolidine-2- carboxamide |
| 203 | 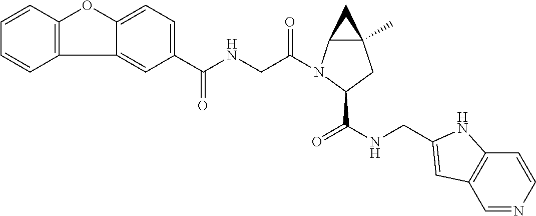 [SMILES: C[C@@]12C[C@@H]1N(C(=O)CNC(=O)c1ccc3oc4ccccc4c3c1)[C@H](C(=O)NCc1cc3cnccc3[nH]1)C2] | (1S,3S,5S)-N-((1H-pyrrolo[3,2- c]pyridin-2-yl)methyl)-2- ((dibenzo[b,d]furan-2- carbonyl)glycyl)-5-methyl-2- azabicyclo[3.1.0]hexane-3- carboxamide |
| 204 | 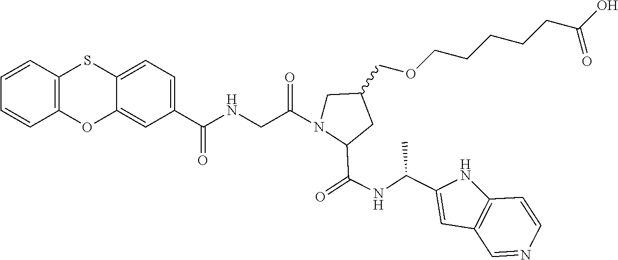 [SMILES: C[C@@H](NC(=O)C1CC(COCCCCCC(=O)O)CN1C(=O)CNC(=O)c1ccc2c(c1)Oc1ccccc1S2)c1cc2cnccc2[nH]1] | 6-(((5S)-5-(((R)-1-(1H- pyrrolo[3,2-c]pyridin-2- yl)ethyl)carbamoyl)-1- ((phenoxathiine-3- carbonyl)glycyl)pyrrolidin-3- yl)methoxy)hexanoic acid |
| 205 | 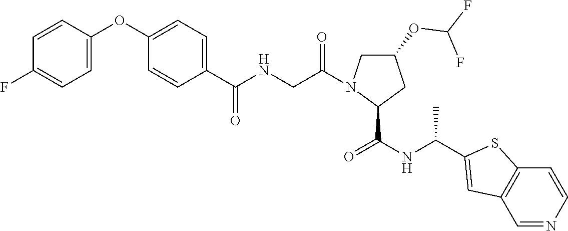 [SMILES: C[C@@H](NC(=O)[C@@H]1C[C@@H](OC(F)F)CN1C(=O)CNC(=O)c1ccc(Oc2ccc(F)cc2)cc1)c1cc2cnccc2s1] | (2S,4R)-4-(difluoromethoxy)-1- ((4-(4- fluorophenoxy)benzoyl)glycyl)- N-((R)-1-(thieno[3,2-c]pyridin-2- yl)ethyl)pyrrolidine-2- carboxamide |
| 206 | 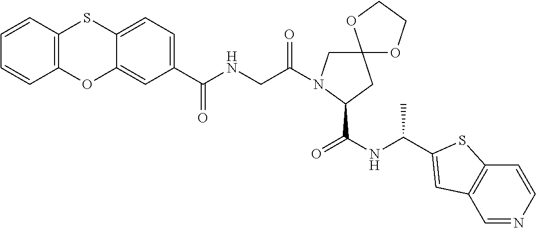 [SMILES: C[C@@H](NC(=O)[C@@H]1CC2(CN1C(=O)CNC(=O)c1ccc3c(c1)Oc1ccccc1S3)OCCO2)c1cc2cnccc2s1] | (S)-7-((phenoxathiine-3- carbonyl)glycyl)-N-((R)-1- (thieno[3,2-c]pyridin-2-yl)ethyl)- 1,4-dioxa-7-azaspiro[4.4]nonane- 8-carboxamide |
| 207 | 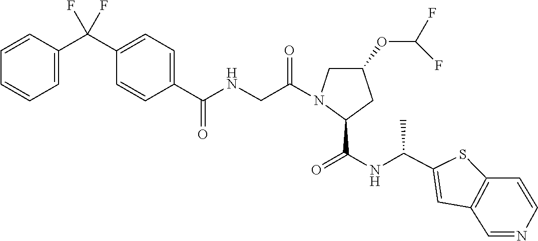 [SMILES: C[C@@H](NC(=O)[C@@H]1C[C@@H](OC(F)F)CN1C(=O)CNC(=O)c1ccc(C(F)(F)c2ccccc2)cc1)c1cc2cnccc2s1] | (2S,4R)-1-((9,9-difluoro-9H- fluorene-3-carbonyl)glycyl)-4- (difluoromethoxy)-N-((R)-1- (thieno[3,2-c]pyridin-2- yl)ethyl)pyrrolidine-2- carboxamide |
| 208 | 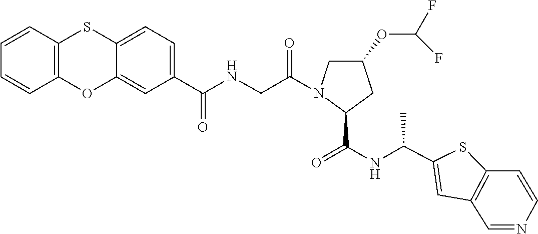 [SMILES: C[C@@H](NC(=O)[C@@H]1C[C@@H](OC(F)F)CN1C(=O)CNC(=O)c1ccc2c(c1)Oc1ccccc1S2)c1cc2cnccc2s1] | (2S,4R)-4-(difluoromethoxy)-1- ((phenoxathiine-3- carbonyl)glycyl)-N-((R)-1- (thieno[3,2-c]pyridin-2- yl)ethyl)pyrrolidine-2- carboxamide |
| 209 | 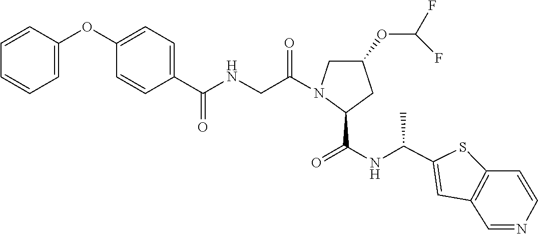 [SMILES: C[C@@H](NC(=O)[C@@H]1C[C@@H](OC(F)F)CN1C(=O)CNC(=O)c1ccc(Oc2ccccc2)cc1)c1cc2cnccc2s1] | (2S,4R)-4-(difluoromethoxy)-1- ((4-phenoxybenzoyl)glycyl)-N- ((R)-1-(thieno[3,2-c]pyridin-2- yl)ethyl)pyrrolidine-2- carboxamide |
| 210 | 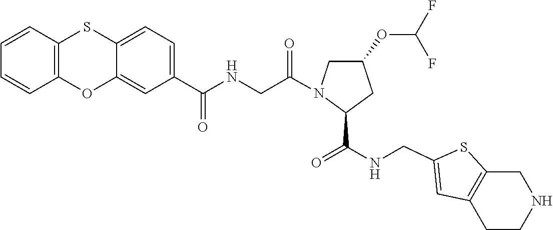 [SMILES: O=C(NCC(=O)N1C[C@H](OC(F)F)C[C@H]1C(=O)NCc1cc2c(s1)CNCC2)c1ccc2c(c1)Oc1ccccc1S2] | (2S,4R)-4-(difluoromethoxy)-1- ((phenoxathiine-3- carbonyl)glycyl)-N-((4,5,6,7- tetrahydrothieno[2,3-c]pyridin-2- yl)methyl)pyrrolidine-2- carboxamide |
| 211 | 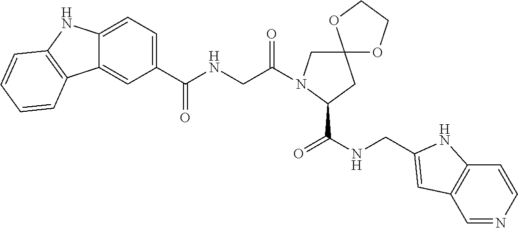 [SMILES: O=C(NCC(=O)N1CC2(C[C@H]1C(=O)NCc1cc3cnccc3[nH]1)OCCO2)c1ccc2[nH]c3ccccc3c2c1] | (S)-N-((1H-pyrrolo[3,2-c]pyridin- 2-yl)methyl)-7-((9H-carbazole-3- carbonyl)glycyl)-1,4-dioxa-7- azaspiro[4.4]nonane-8- carboxamide |
| 212 | 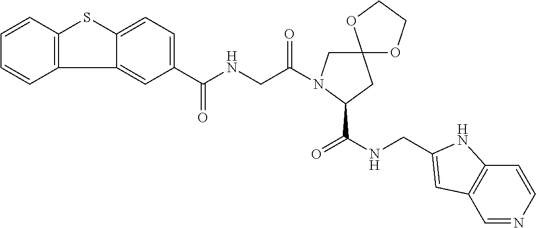 [SMILES: O=C(NCC(=O)N1CC2(C[C@H]1C(=O)NCc1cc3cnccc3[nH]1)OCCO2)c1ccc2sc3ccccc3c2c1] | (S)-N-((1H-pyrrolo[3,2-c]pyridin- 2-yl)methyl)-7- ((dibenzo[b,d]thiophene-2- carbonyl)glycyl)-1,4-dioxa-7- azaspiro[4.4]nonane-8- carboxamide |
| 213 | 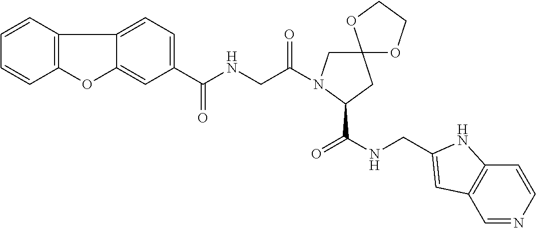 [SMILES: O=C(NCC(=O)N1CC2(C[C@H]1C(=O)NCc1cc3cnccc3[nH]1)OCCO2)c1ccc2c(c1)oc1ccccc12] | (S)-N-((1H-pyrrolo[3,2-c]pyridin- 2-yl)methyl)-7- ((dibenzo[b,d]furan-3- carbonyl)glycyl)-1,4-dioxa-7- azaspiro[4.4]nonane-8- carboxamide |
| 214 | 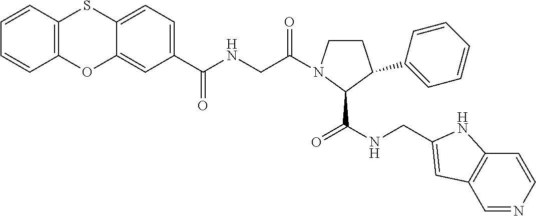 [SMILES: O=C(NCC(=O)N1CC[C@H](c2ccccc2)[C@H]1C(=O)NCc1cc2cnccc2[nH]1)c1ccc2c(c1)Oc1ccccc1S2] | (2S,3R)-N-((1H-pyrrolo[3,2- c]pyridin-2-yl)methyl)-1- ((phenoxathiine-3- carbonyl)glycyl)-3- phenylpyrrolidine-2-carboxamide |
| 215 | 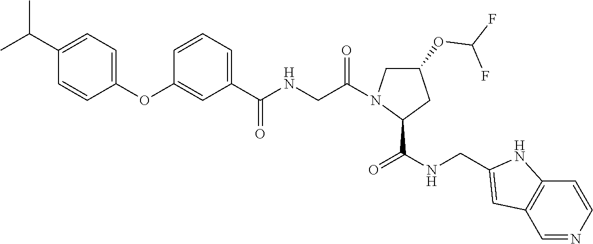 [SMILES: CC(C)c1ccc(Oc2cccc(C(=O)NCC(=O)N3C[C@H](OC(F)F)C[C@H]3C(=O)NCc3cc4cnccc4[nH]3)c2)cc1] | (2S,4R)-N-((1H-pyrrolo[3,2- c]pyridin-2-yl)methyl)-4- (difluoromethoxy)-1-((3-(4- isopropylphenoxy)benzoyl) glycyl)pyrrolidine-2-carboxamide |
| 216 | 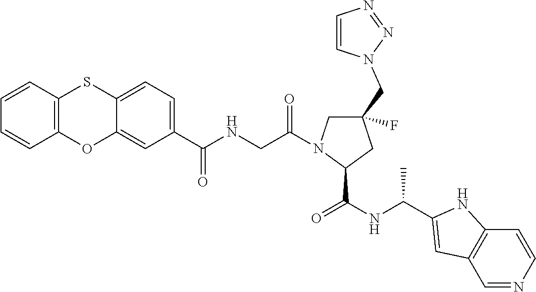 [SMILES: C[C@@H](NC(=O)[C@@H]1C[C@](F)(Cn2ccnn2)CN1C(=O)CNC(=O)c1ccc2c(c1)Oc1ccccc1S2)c1cc2cnccc2[nH]1] | (2S,4R)-N-((1H-1,2,3-triazol-1- yl)methyl)-N-((R)-1-(1H- pyrrolo[3,2-c]pyridin-2-yl)ethyl)- 4-fluoro-1-((phenoxathiine-3- carbonyl)glycyl)pyrrolidine-2- carboxamide |
| 217 | 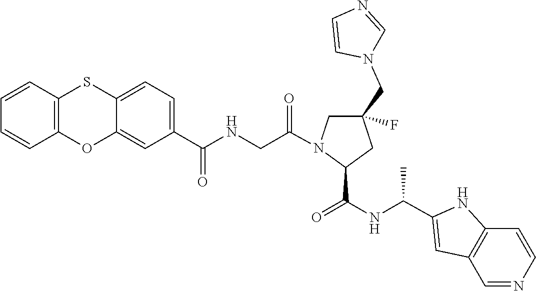 [SMILES: C[C@@H](NC(=O)[C@@H]1C[C@](F)(Cn2ccnc2)CN1C(=O)CNC(=O)c1ccc2c(c1)Oc1ccccc1S2)c1cc2cnccc2[nH]1] | (2S,4S)-4-((1H-imidazol-1- yl)methyl)-N-((R)-1-(1H- pyrrolo[3,2-c]pyridin-2-yl)ethyl)- 4-fluoro-1-((phenoxathiine-3- carbonyl)glycyl)pyrrolidine-2- carboxamide |
| 218 | 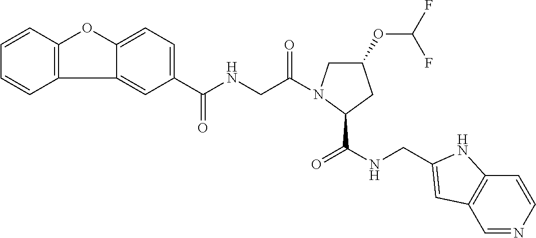 [SMILES: O=C(NCC(=O)N1C[C@H](OC(F)F)C[C@H]1C(=O)NCc1cc2cnccc2[nH]1)c1ccc2oc3ccccc3c2c1] | (2S,4R)-N-((1H-pyrrolo[3,2- c]pyridin-2-yl)methyl)-1- ((dibenzo[b,d]furan-2- carbonyl)glycyl)-4- (difluoromethoxy)pyrrolidine-2- carboxamide |
| 219 | 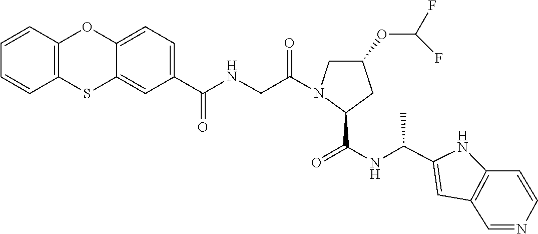 [SMILES: C[C@@H](NC(=O)[C@@H]1C[C@@H](OC(F)F)CN1C(=O)CNC(=O)c1ccc2c(c1)Sc1ccccc1O2)c1cc2cnccc2[nH]1] | (2S,4R)-N-((R)-1-(1H- pyrrolo[3,2-c]pyridin-2-yl)ethyl)- 4-(difluoromethoxy)-1- ((phenoxathiine-3- carbonyl)glycyl)pyrrolidine-2- carboxamide |
| 220 | 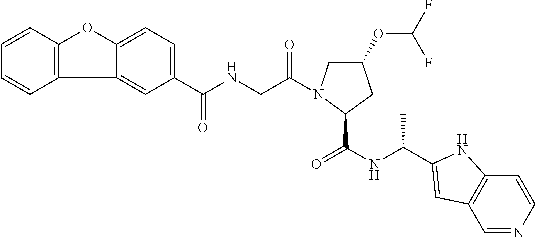 [SMILES: C[C@@H](NC(=O)[C@@H]1C[C@@H](OC(F)F)CN1C(=O)CNC(=O)c1ccc2oc3ccccc3c2c1)c1cc2cnccc2[nH]1] | (2S,4R)-N-((R)-1-(1H- pyrrolo[3,2-c]pyridin-2-yl)ethyl)- 1-((dibenzo[b,d]furan-2- carbonyl)glycyl)-4- (difluoromethoxy)pyrrolidine-2- carboxamide |
| 221 | 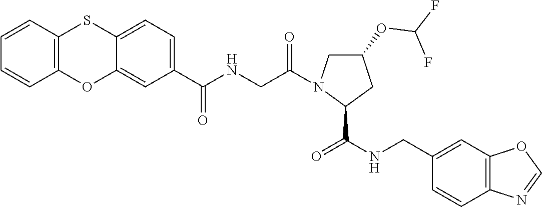 [SMILES: O=C(NCC(=O)N1C[C@H](OC(F)F)C[C@H]1C(=O)NCc1ccc2ncoc2c1)c1ccc2c(c1)Oc1ccccc1S2] | (2S,4R)-N-(benzo[d]oxazol-6- ylmethyl)-4-(difluoromethoxy)-1- ((phenoxathiine-3- carbonyl)glycyl)pyrrolidine-2- carboxamide |
| 222 | 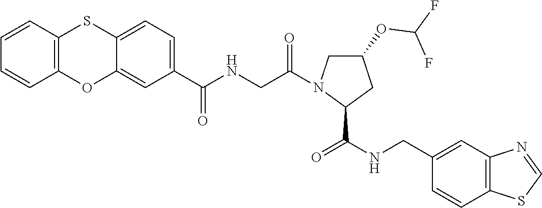 [SMILES: O=C(NCC(=O)N1C[C@H](OC(F)F)C[C@H]1C(=O)NCc1ccc2scnc2c1)c1ccc2c(c1)Oc1ccccc1S2] | (2S,4R)-N-(benzo[d]thiazol-5- ylmethyl)-4-(difluoromethoxy)-1- ((phenoxathiine-3- carbonyl)glycyl)pyrrolidine-2- carboxamide |
| 223 | 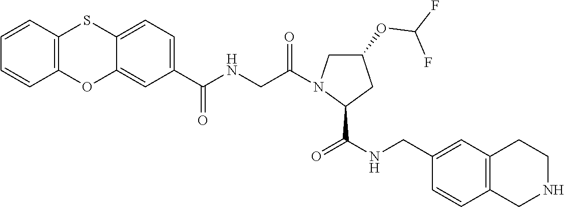 [SMILES: O=C(NCC(=O)N1C[C@H](OC(F)F)C[C@H]1C(=O)NCc1ccc2c(c1)CCNC2)c1ccc2c(c1)Oc1ccccc1S2] | (2S,4R)-4-(difluoromethoxy)-1- ((phenoxathiine-3- carbonyl)glycyl)-N-((1,2,3,4- tetrahydroisoquinolin-6- yl)methyl)pyrrolidine-2- carboxamide |
| 224 | 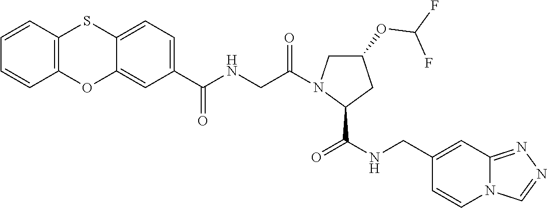 [SMILES: O=C(NCC(=O)N1C[C@H](OC(F)F)C[C@H]1C(=O)NCc1ccn2cnnc2c1)c1ccc2c(c1)Oc1ccccc1S2] | (2S,4R)-N-([1,2,4]triazolo[4,3- a]pyridin-7-ylmethyl)-4- (difluoromethoxy)-1- ((phenoxathiine-3- carbonyl)glycyl)pyrrolidine-2- carboxamide |
| 225 | 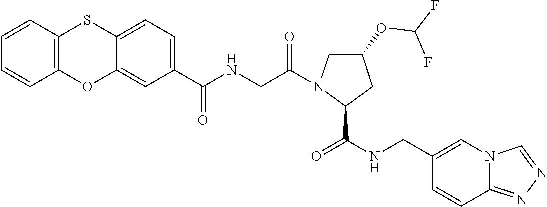 [SMILES: O=C(NCC(=O)N1C[C@H](OC(F)F)C[C@H]1C(=O)NCc1ccc2nncn2c1)c1ccc2c(c1)Oc1ccccc1S2] | (2S,4R)-N-([1,2,4]triazolo[4,3- a]pyridin-6-ylmethyl)-4- (difluoromethoxy)-1- ((phenoxathiine-3- carbonyl)glycyl)pyrrolidine-2- carboxamide |
| 226 | 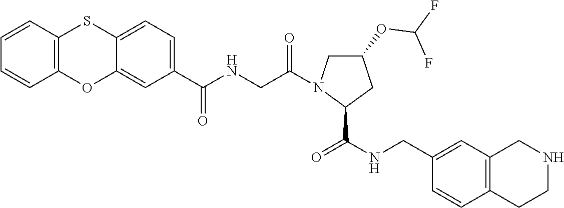 [SMILES: O=C(NCC(=O)N1C[C@H](OC(F)F)C[C@H]1C(=O)NCc1ccc2c(c1)CNCC2)c1ccc2c(c1)Oc1ccccc1S2] | (2S,4R)-4-(difluoromethoxy)-1- ((phenoxathiine-3- carbonyl)glycyl)-N-((1,2,3,4- tetrahydroisoquinolin-7- yl)methyl)pyrrolidine-2- carboxamide |
| 227 | 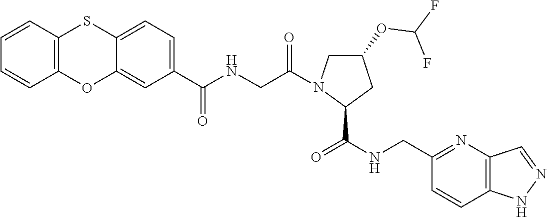 [SMILES: O=C(NCC(=O)N1C[C@H](OC(F)F)C[C@H]1C(=O)NCc1ccc2[nH]ncc2n1)c1ccc2c(c1)Oc1ccccc1S2] | (2S,4R)-N-((1H-pyrazolo[4,3- b]pyridin-5-yl)methyl)-4- (difluoromethoxy)-1- ((phenoxathiine-3- carbonyl)glycyl)pyrrolidine-2- carboxamide |
| 228 | 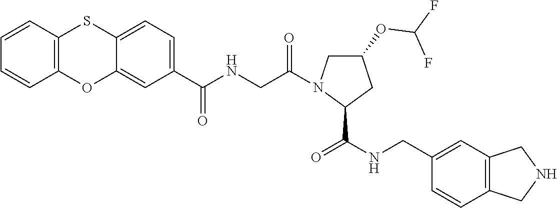 [SMILES: O=C(NCC(=O)N1C[C@H](OC(F)F)C[C@H]1C(=O)NCc1ccc2c(c1)CNC2)c1ccc2c(c1)Oc1ccccc1S2] | (2S,4R)-4-(difluoromethoxy)-N- (isoindolin-5-ylmethyl)-1- ((phenoxathiine-3- carbonyl)glycyl)pyrrolidine-2- carboxamide |
| 229 | 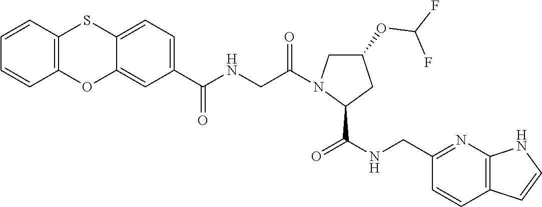 [SMILES: O=C(NCC(=O)N1C[C@H](OC(F)F)C[C@H]1C(=O)NCc1ccc2cc[nH]c2n1)c1ccc2c(c1)Oc1ccccc1S2] | (2S,4R)-N-((1H-pyrrolo[2,3- b]pyridin-6-yl)methyl)-4- (difluoromethoxy)-1- ((phenoxathiine-3- carbonyl)glycyl)pyrrolidine-2- carboxamide |
| 230 | 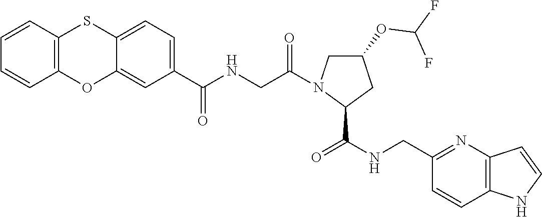 [SMILES: O=C(NCC(=O)N1C[C@H](OC(F)F)C[C@H]1C(=O)NCc1ccc2[nH]ccc2n1)c1ccc2c(c1)Oc1ccccc1S2] | (2S,4R)-N-((1H-pyrrolo[3,2- b]pyridin-5-yl)methyl)-4- (difluoromethoxy)-1- ((phenoxathiine-3- carbonyl)glycyl)pyrrolidine-2- carboxamide |
| 231 | 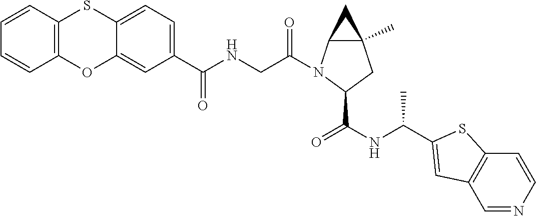 [SMILES: C[C@@H](NC(=O)[C@@H]1C[C@]2(C)C[C@@H]2N1C(=O)CNC(=O)c1ccc2c(c1)Oc1ccccc1S2)c1cc2cnccc2s1] | (1S,3S,5S)-5-methyl-2- ((phenoxathiine-3- carbonyl)glycyl)-N-((R)-1- (thieno[3,2-c]pyridin-2-yl)ethyl)- 2-azabicyclo[3.1.0]hexane-3- carboxamide |
| 232 | 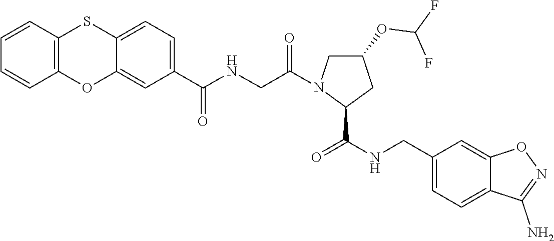 [SMILES: Nc1noc2cc(CNC(=O)[C@@H]3C[C@@H](OC(F)F)CN3C(=O)CNC(=O)c3ccc4c(c3)Oc3ccccc3S4)ccc12] | (2S,4R)-N-((3- aminobenzo[d]isoxazol-6- yl)methyl)-4-(difluoromethoxy)- 1-((phenoxathiine-3- carbonyl)glycyl)pyrrolidine-2- carboxamide |
| 233 | 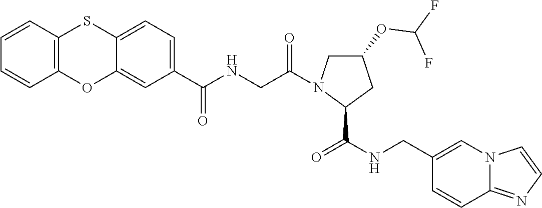 [SMILES: O=C(NCC(=O)N1C[C@H](OC(F)F)C[C@H]1C(=O)NCc1ccc2nccn2c1)c1ccc2c(c1)Oc1ccccc1S2] | (2S,4R)-4-(difluoromethoxy)-N- (imidazo[1,2-a]pyridin-6- ylmethyl)-1-((phenoxathiine-3- carbonyl)glycyl)pyrrolidine-2- carboxamide |
| 234 | 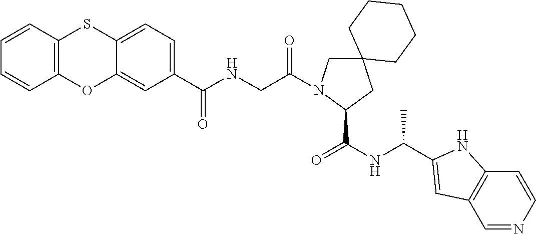 [SMILES: C[C@@H](NC(=O)[C@@H]1CC2(CCCCC2)CN1C(=O)CNC(=O)c1ccc2c(c1)Oc1ccccc1S2)c1cc2cnccc2[nH]1] | (S)-N-((R)-1-(1H-pyrrolo[3,2- c]pyridin-2-yl)ethyl)-2- ((phenoxathiine-3- carbonyl)glycyl)-2- azaspiro[4.5]decane-3- carboxamide |
| 235 | 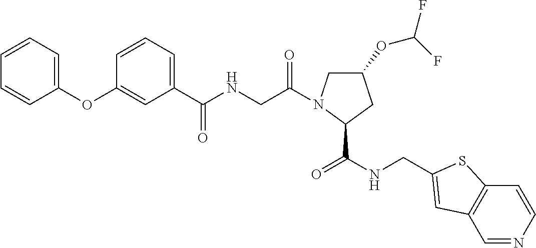 [SMILES: O=C(NCC(=O)N1C[C@H](OC(F)F)C[C@H]1C(=O)NCc1cc2cnccc2s1)c1cccc(Oc2ccccc2)c1] | (2S,4R)-4-(difluoromethoxy)-1- ((3-phenoxybenzoyl)glycyl)-N- (thieno[3,2-c]pyridin-2- ylmethyl)pyrrolidine-2- carboxamide |
| 236 | 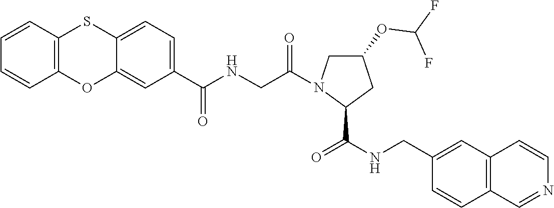 [SMILES: O=C(NCC(=O)N1C[C@H](OC(F)F)C[C@H]1C(=O)NCc1ccc2cnccc2c1)c1ccc2c(c1)Oc1ccccc1S2] | (2S,4R)-4-(difluoromethoxy)-N- (isoquinolin-6-ylmethyl)-1- ((phenoxathiine-3- carbonyl)glycyl)pyrrolidine-2- carboxamide |
| 237 | 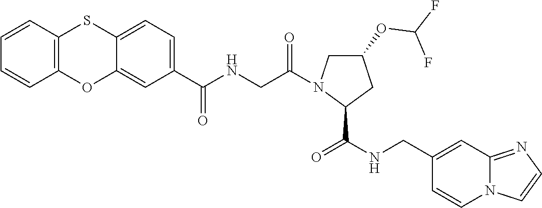 [SMILES: O=C(NCC(=O)N1C[C@H](OC(F)F)C[C@H]1C(=O)NCc1ccn2ccnc2c1)c1ccc2c(c1)Oc1ccccc1S2] | (2S,4R)-4-(difluoromethoxy)-N- (imidazo[1,2-a]pyridin-7- ylmethyl)-1-((phenoxathiine-3- carbonyl)glycyl)pyrrolidine-2- carboxamide |
| 238 | 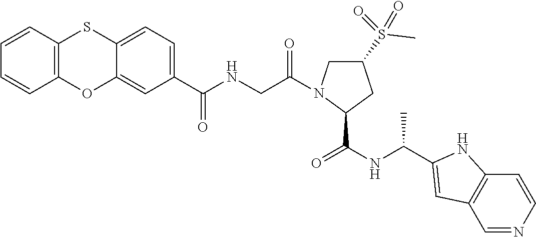 [SMILES: C[C@@H](NC(=O)[C@@H]1C[C@@H](S(C)(=O)=O)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 4-(methylsulfonyl)-1- ((phenoxathiine-3- carbonyl)glycyl)pyrrolidine-2- carboxamide |
| 239 | 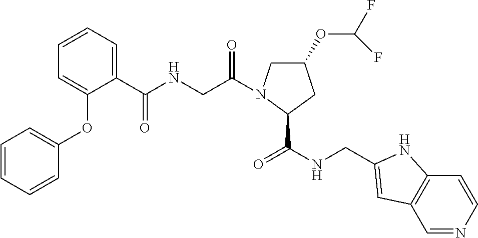 [SMILES: O=C(NCC(=O)N1C[C@H](OC(F)F)C[C@H]1C(=O)NCc1cc2cnccc2[nH]1)c1ccccc1Oc1ccccc1] | (2S,4R)-N-((1H-pyrrolo[3,2- c]pyridin-2-yl)methyl)-4- (difluoromethoxy)-1-((2- phenoxybenzoyl)glycyl)pyrrolidine- 2-carboxamide |
| 240 | 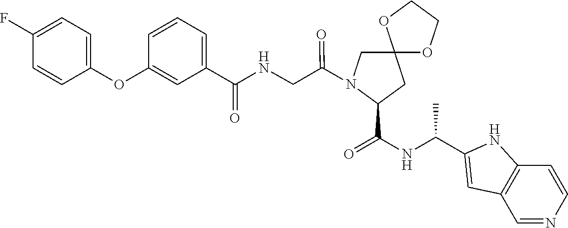 [SMILES: C[C@@H](NC(=O)[C@@H]1CC2(CN1C(=O)CNC(=O)c1cccc(Oc3ccc(F)cc3)c1)OCCO2)c1cc2cnccc2[nH]1] | (S)-N-((R)-1-(1H-pyrrolo[3,2- c]pyridin-2-yl)ethyl)-7-((3-(4- fluorophenoxy)benzoyl)glycyl)- 1,4-dioxa-7-azaspiro[4.4]nonane- 8-carboxamide |
| 241 | 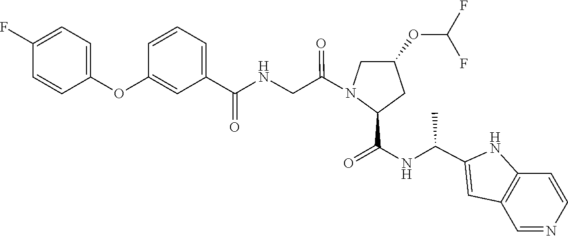 [SMILES: C[C@@H](NC(=O)[C@@H]1C[C@@H](OC(F)F)CN1C(=O)CNC(=O)c1cccc(Oc2ccc(F)cc2)c1)c1cc2cnccc2[nH]1] | (2S,4R)-N-((R)-1-(1H- pyrrolo[3,2-c]pyridin-2-yl)ethyl)- 4-(difluoromethoxy)-1-((3-(4- fluorophenoxy)benzoyl)glycyl) pyrrolidine-2-carboxamide |
| 242 | 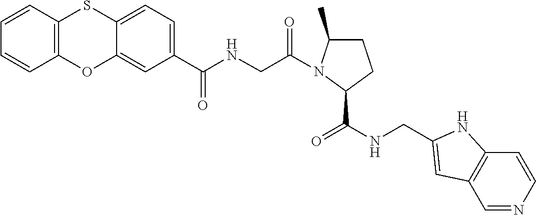 [SMILES: C[C@H]1CC[C@@H](C(=O)NCc2cc3cnccc3[nH]2)N1C(=O)CNC(=O)c1ccc2c(c1)Oc1ccccc1S2] | (2S,5S)-N-((1H-pyrrolo[2,3- c]pyridin-2-yl)methyl)-5-methyl- 1-((phenoxathiine-3- carbonyl)glycyl)pyrrolidine-2- carboxamide |
| 243 | 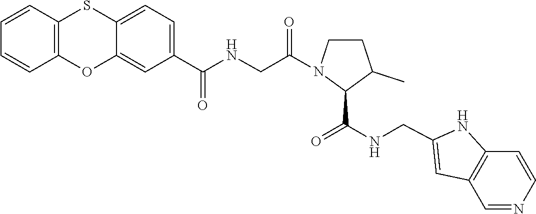 [SMILES: CC1CCN(C(=O)CNC(=O)c2ccc3c(c2)Oc2ccccc2S3)[C@@H]1C(=O)NCc1cc2cnccc2[nH]1] | (2S)-N-((1H-pyrrolo[3,2- c]pyridin-2-yl)methyl)-3-methyl- 1-((phenoxathiine-3- carbonyl)glycyl)pyrrolidine-2- carboxamide |
| 244 | 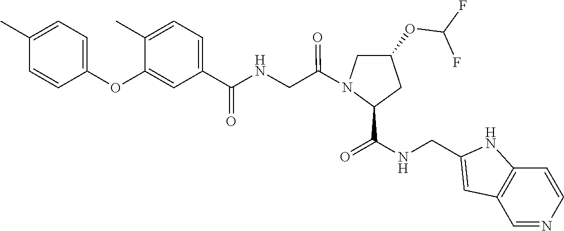 [SMILES: Cc1ccc(Oc2cc(C(=O)NCC(=O)N3C[C@H](OC(F)F)C[C@H]3C(=O)NCc3cc4cnccc4[nH]3)ccc2C)cc1] | (2S,4R)-N-((1H-pyrrolo[3,2- c]pyridin-2-yl)methyl)-4- (difluoromethoxy)-1-((4-methyl- 3-(p-tolyloxy)benzoyl)glycyl) pyrrolidine-2-carboxamide |
| 245 | 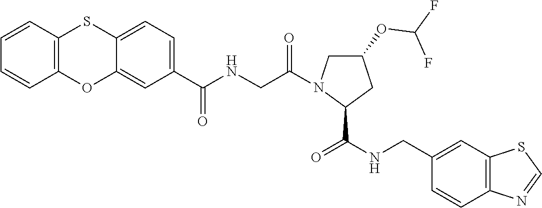 [SMILES: O=C(NCC(=O)N1C[C@H](OC(F)F)C[C@H]1C(=O)NCc1ccc2ncsc2c1)c1ccc2c(c1)Oc1ccccc1S2] | (2S,4R)-N-(benzo[d]thiazol-6- ylmethyl)-4-(difluoromethoxy)-1- ((phenoxathiine-3- carbonyl)glycyl)pyrrolidine-2- carboxamide |
| 246 | 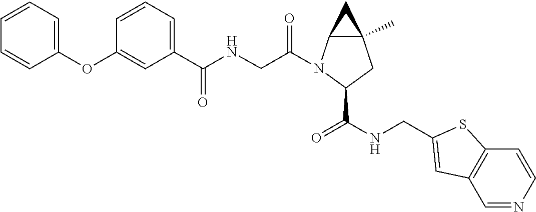 [SMILES: C[C@@]12C[C@@H]1N(C(=O)CNC(=O)c1cccc(Oc3ccccc3)c1)[C@H](C(=O)NCc1cc3cnccc3s1)C2] | (1S,3S,5S)-5-methyl-2-((3- phenoxybenzoyl)glycyl)-N- (thieno[3,2-c]pyridin-2-ylmethyl)- 2-azabicyclo[3.1.0]hexane-3- carboxamide |
| 247 | 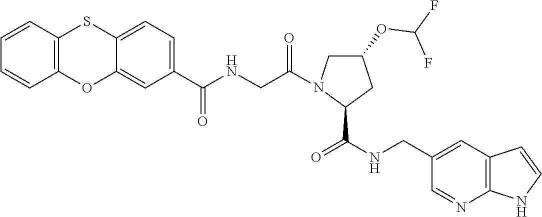 [SMILES: O=C(NCC(=O)N1C[C@H](OC(F)F)C[C@H]1C(=O)NCc1cnc2[nH]ccc2c1)c1ccc2c(c1)Oc1ccccc1S2] | (2S,4R)-N-((1H-pyrrolo[3,2- b]pyridin-5-yl)methyl)-4- (difluoromethoxy)-1- ((phenoxathiine-3- carbonyl)glycyl)pyrrolidine-2- carboxamide |
| 248 | 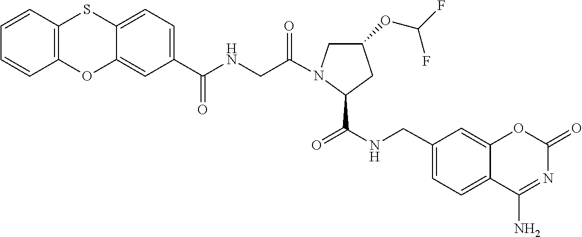 [SMILES: Nc1nc(=O)oc2cc(CNC(=O)[C@@H]3C[C@@H](OC(F)F)CN3C(=O)CNC(=O)c3ccc4c(c3)Oc3ccccc3S4)ccc12] | (2S,4R)-N-((4-amino-2-oxo-2H- benzo[e][1,3]oxazin-7-yl)methyl)- 4-(difluoromethoxy)-1- ((phenoxathiine-3- carbonyl)glycyl)pyrrolidine-2- carboxamide |
| 249 | 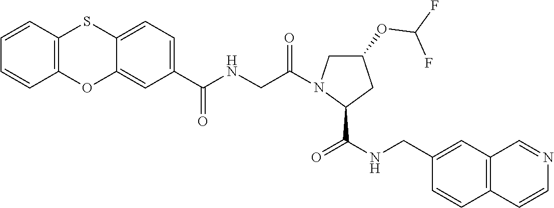 [SMILES: O=C(NCC(=O)N1C[C@H](OC(F)F)C[C@H]1C(=O)NCc1ccc2ccncc2c1)c1ccc2c(c1)Oc1ccccc1S2] | (2S,4R)-4-(difluoromethoxy)-N- (isoquinolin-7-ylmethyl)-1- ((phenoxathiine-3- carbonyl)glycyl)pyrrolidine-2- carboxamide |
| 250 | 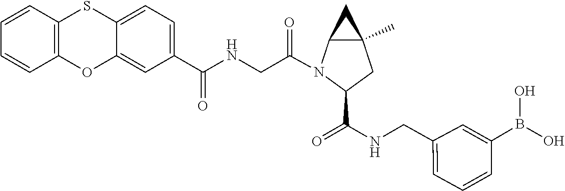 [SMILES: C[C@@]12C[C@@H]1N(C(=O)CNC(=O)c1ccc3c(c1)Oc1ccccc1S3)[C@H](C(=O)NCc1cccc(B(O)O)c1)C2] | (3-(((1S,3S,5S)-5-methyl-2- ((phenoxathiine-3- carbonyl)glycyl)-2- azabicyclo[3.1.0]hexane-3- carboxamido)methyl)phenyl) boronic acid |
| 251 | 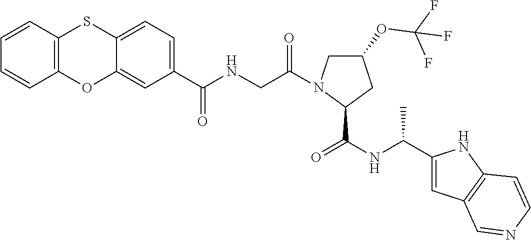 [SMILES: C[C@@H](NC(=O)[C@@H]1C[C@@H](OC(F)(F)F)CN1C(=O)CNC(=O)c1ccc2c(c1)Oc1ccccc1S2)c1cc2cnccc2[nH]1] | (2S,4R)-N-((R)-1-(1H- pyrrolo[3,2-c]pyridin-2-yl)ethyl)- 1-((phenoxathiine-3- carbonyl)glycyl)-4- (trifluoromethoxy)pyrrolidine-2- carboxamide |
| 252 | 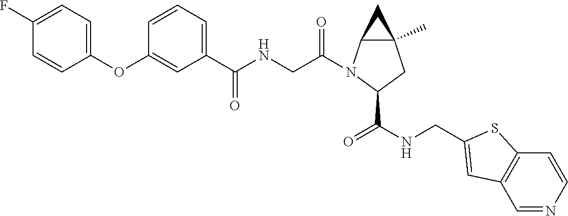 [SMILES: C[C@@]12C[C@@H]1N(C(=O)CNC(=O)c1cccc(Oc3ccc(F)cc3)c1)[C@H](C(=O)NCc1cc3cnccc3s1)C2] | (1S,3S,5S)-2-((3-(4- fluorophenoxy)benzoyl)glycyl)-5- methyl-N-(thieno[3,2-c]pyridin-2- ylmethyl)-2- azabicyclo[3.1.0]hexane-3- carboxamide |
| 253 | 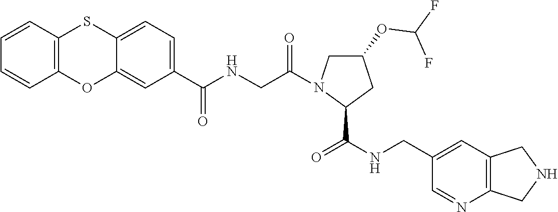 [SMILES: O=C(NCC(=O)N1C[C@H](OC(F)F)C[C@H]1C(=O)NCc1cnc2c(c1)CNC2)c1ccc2c(c1)Oc1ccccc1S2] | (2S,4R)-4-(difluoromethoxy)-N- ((6,7-dihydro-5H-pyrrolo[3,4- b]pyridin-3-yl)methyl)-1- ((phenoxathiine-3- carbonyl)glycyl)pyrrolidine-2- carboxamide |
| 254 | 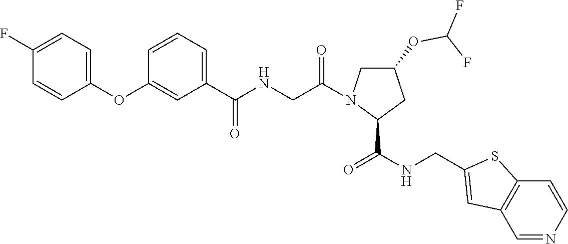 [SMILES: O=C(NCC(=O)N1C[C@H](OC(F)F)C[C@H]1C(=O)NCc1cc2cnccc2s1)c1cccc(Oc2ccc(F)cc2)c1] | (2S,4R)-4-(difluoromethoxy)-1- ((3-(4- fluorophenoxy)benzoyl)glycyl)- N-(thieno[3,2-c]pyridin-2- ylmethyl)pyrrolidine-2- carboxamide |
| 255 | 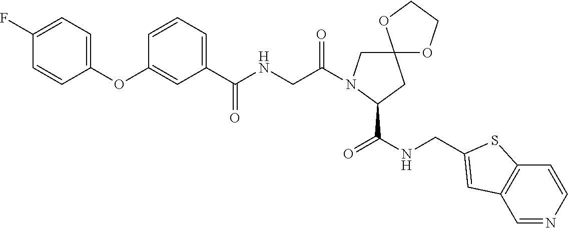 [SMILES: O=C(NCC(=O)N1CC2(C[C@H]1C(=O)NCc1cc3cnccc3s1)OCCO2)c1cccc(Oc2ccc(F)cc2)c1] | (S)-7-((3-(4- fluorophenoxy)benzoyl)glycyl)- N-(thieno[3,2-c]pyridin-2- ylmethyl)-1,4-dioxa-7- azaspiro[4.4]nonane-8- carboxamide |
| 256 | 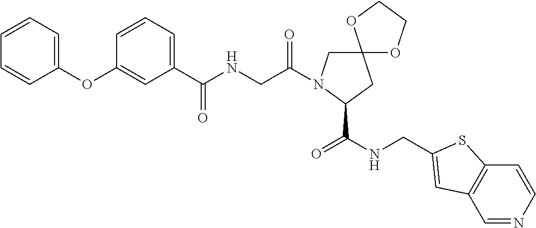 [SMILES: O=C(NCC(=O)N1CC2(C[C@H]1C(=O)NCc1cc3cnccc3s1)OCCO2)c1cccc(Oc2ccccc2)c1] | (S)-7-((3- phenoxybenzoyl)glycyl)-N- (thieno[3,2-c]pyridin-2-ylmethyl)- 1,4-dioxa-7-azaspiro[4.4]nonane- 8-carboxamide |
| 257 | 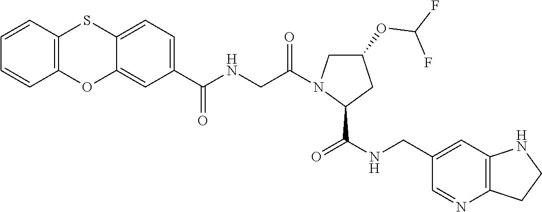 [SMILES: O=C(NCC(=O)N1C[C@H](OC(F)F)C[C@H]1C(=O)NCc1cnc2c(c1)NCC2)c1ccc2c(c1)Oc1ccccc1S2] | (2S,4R)-4-(difluoromethoxy)-N- ((2,3-dihydro-1H-pyrrolo[3,2- b]pyridin-6-yl)methyl)-1- ((phenoxathiine-3- carbonyl)glycyl)pyrrolidine-2- carboxamide |
| 258 | 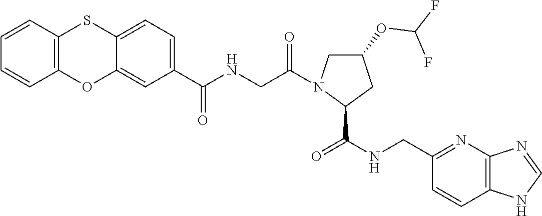 [SMILES: O=C(NCC(=O)N1C[C@H](OC(F)F)C[C@H]1C(=O)NCc1ccc2[nH]cnc2n1)c1ccc2c(c1)Oc1ccccc1S2] | (2S,4R)-N-((1H-imidazo[4,5- b]pyridin-5-yl)methyl)-4- (difluoromethoxy)-1- ((phenoxathiine-3- carbonyl)glycyl)pyrrolidine-2- carboxamide |
| 259 | 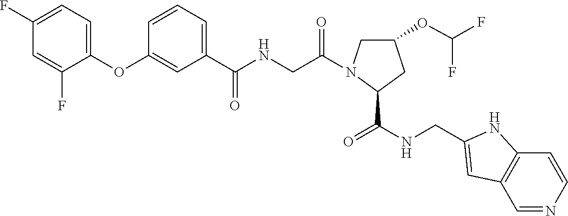 [SMILES: O=C(NCC(=O)N1C[C@H](OC(F)F)C[C@H]1C(=O)NCc1cc2cnccc2[nH]1)c1cccc(Oc2ccc(F)cc2F)c1] | (2S,4R)-N-((1H-pyrrolo[3,2- c]pyridin-2-yl)methyl)-4- (difluoromethoxy)-1-((3-(2,4- difluorophenoxy)benzoyl)glcyl) pyrrolidine-2-carboxamide |
| 260 | 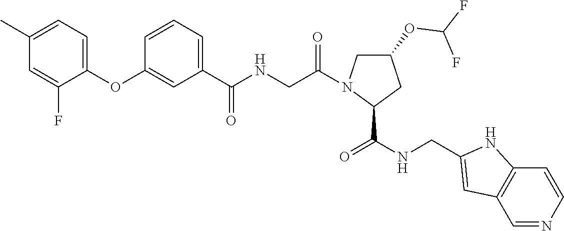 [SMILES: Cc1ccc(Oc2cccc(C(=O)NCC(=O)N3C[C@H](OC(F)F)C[C@H]3C(=O)NCc3cc4cnccc4[nH]3)c2)c(F)c1] | (2S,4R)-N-((1H-pyrrolo[3,2- c]pyridin-2-yl)methyl)-4- (difluoromethoxy)-1-((3-(2- fluoro-4- methylphenoxy)benzoyl)glycyl) pyrrolidine-2-carboxamide |
| 261 | 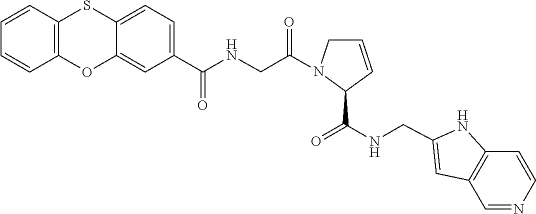 [SMILES: O=C(NCC(=O)N1CC=C[C@H]1C(=O)NCc1cc2cnccc2[nH]1)c1ccc2c(c1)Oc1ccccc1S2] | (S)-N-((1H-pyrrolo[3,2-c]pyridin- 2-yl)methyl)-1-((phenoxathiine-3- carbonyl)glycyl)-2,5-dihydro-1H- pyrrole-2-carboxamide |
| 262 | 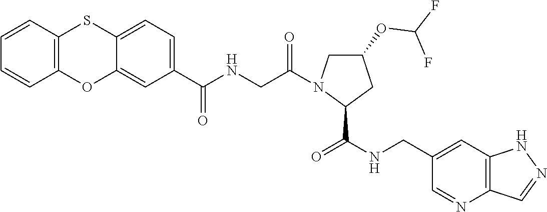 [SMILES: O=C(NCC(=O)N1C[C@H](OC(F)F)C[C@H]1C(=O)NCc1cnc2cn[nH]c2c1)c1ccc2c(c1)Oc1ccccc1S2] | (2S,4R)-N-((1H-pyrrolo[4,3- b]pyridin-6-yl)methyl)-4- (difluoromethoxy)-1- ((phenoxathiine-3- carbonyl)glycyl)pyrrolidine-2- carboxamide |
| 263 | 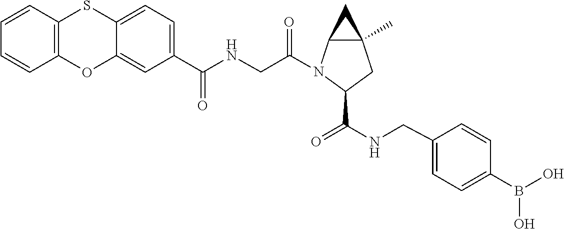 [SMILES: C[C@@]12C[C@@H]1N(C(=O)CNC(=O)c1ccc3c(c1)Oc1ccccc1S3)[C@H](C(=O)NCc1ccc(B(O)O)cc1)C2] | (4-(((1S,3S,5S)-5-methyl-2- ((phenoxathiine-3- carbonyl)glycyl)-2- azabicyclo[3.1.0]hexane-3- carboxamido)methyl)phenyl) boronic acid |
| 264 | 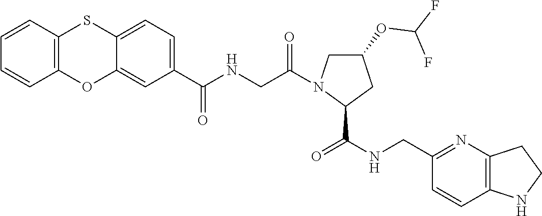 [SMILES: O=C(NCC(=O)N1C[C@H](OC(F)F)C[C@H]1C(=O)NCc1ccc2c(n1)CCN2)c1ccc2c(c1)Oc1ccccc1S2] | (2S,4R)-4-(difluoromethoxy)-N- ((2,3-dihydro-1H-pyrrolo[3,2- b]pyridin-5-yl)methyl)-1- ((phenoxathiine-3- carbonyl)glycyl)pyrrolidine-2- carboxamide |
| 265 | 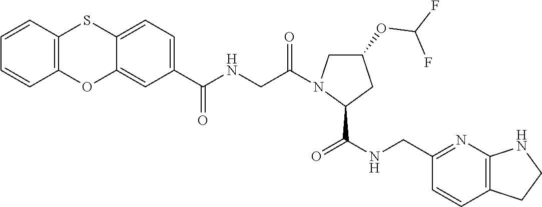 [SMILES: O=C(NCC(=O)N1C[C@H](OC(F)F)C[C@H]1C(=O)NCc1ccc2c(n1)NCC2)c1ccc2c(c1)Oc1ccccc1S2] | (2S,4R)-4-(difluoromethoxy)-N- ((2,3-dihydro-1H-pyrrolo[2,3- b]pyridin-6-yl)methyl)-1- ((phenoxathiine-3- carbonyl)glycyl)pyrrolidine-2- carboxamide |
| 266 | 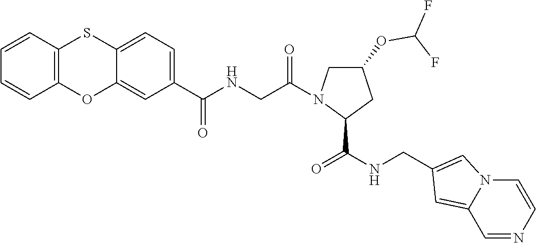 [SMILES: O=C(NCC(=O)N1C[C@H](OC(F)F)C[C@H]1C(=O)NCc1cc2cnccn2c1)c1ccc2c(c1)Oc1ccccc1S2] | (2S,4R)-4-(difluoromethoxy)-1- ((phenoxathiine-3- carbonyl)glycyl)-N-(pyrrolo[1,2- a]pyrazin-7-ylmethyl)pyrrolidine- 2-carboxamide |
| 267 | 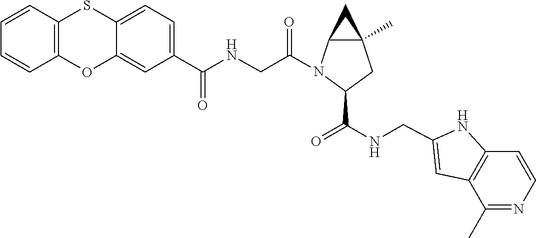 [SMILES: Cc1nccc2[nH]c(CNC(=O)[C@@H]3C[C@]4(C)C[C@@H]4N3C(=O)CNC(=O)c3ccc4c(c3)Oc3ccccc3S4)cc12] | (1S,3S,5S)-5-methyl-N-((4- methyl-1H-pyrrolo[3,2-c]pyridin- 2-yl)methyl)-2-((phenoxathiine-3- carbonyl)glycyl)-2- azabicyclo[3.1.0]hexane-3- carboxamide |
| 268 | 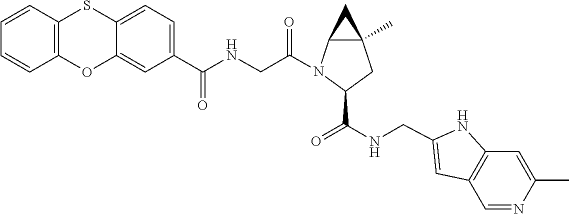 [SMILES: Cc1cc2[nH]c(CNC(=O)[C@@H]3C[C@]4(C)C[C@@H]4N3C(=O)CNC(=O)c3ccc4c(c3)Oc3ccccc3S4)cc2cn1] | (1S,3S,5S)-5-methyl-N-((6- methyl-1H-pyrrolo[3,2-c]pyridin- 2-yl)methyl)-2-((phenoxathiine-3- carbonyl)glycyl)-2- azabicyclo[3.1.0]hexane-3- carboxamide |
| 269 | 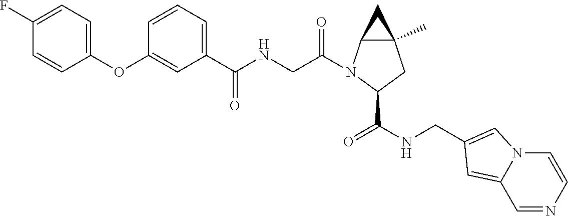 [SMILES: C[C@@]12C[C@@H]1N(C(=O)CNC(=O)c1cccc(Oc3ccc(F)cc3)c1)[C@H](C(=O)NCc1cc3cnccn3c1)C2] | (1S,3S,5S)-2-((3-(4- fluorophenoxy)benzoyl)glycyl)-5- methyl-N-(pyrrolo[1,2-a]pyrazin- 7-ylmethyl)-2- azabicyclo[3.1.0]hexane-3- carboxamide |
| 270 | 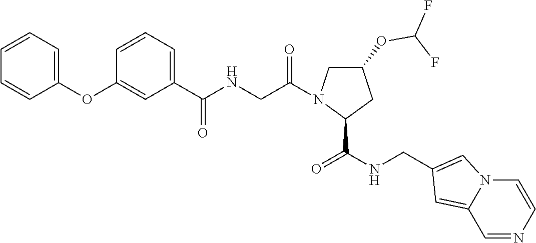 [SMILES: O=C(NCC(=O)N1C[C@H](OC(F)F)C[C@H]1C(=O)NCc1cc2cnccn2c1)c1cccc(Oc2ccccc2)c1] | (2S,4R)-4-(difluoromethoxy)-1- ((3-phenoxybenzoyl)glycyl)-N- (pyrrolo[1,2-a]pyrazin-7- ylmethyl)pyrrolidine-2- carboxamide |
| 271 | 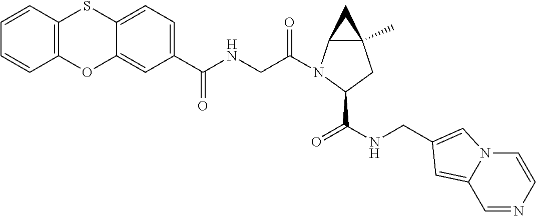 [SMILES: C[C@@]12C[C@@H]1N(C(=O)CNC(=O)c1ccc3c(c1)Oc1ccccc1S3)[C@H](C(=O)NCc1cc3cnccn3c1)C2] | (1S,3S,5S)-5-methyl-2- ((phenoxathiine-3- carbonyl)glycyl)-N-(pyrrolo[1,2- a]pyrazin-7-ylmethyl)-2- azabicyclo[3.1.0]hexane-3- carboxamide |
| 272 | 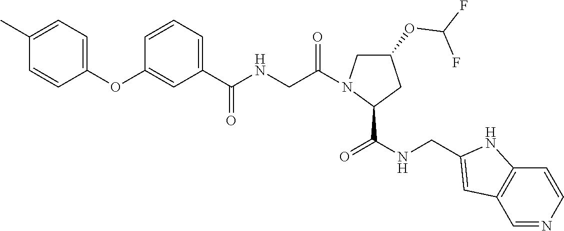 [SMILES: Cc1ccc(Oc2cccc(C(=O)NCC(=O)N3C[C@H](OC(F)F)C[C@H]3C(=O)NCc3cc4cnccc4[nH]3)c2)cc1] | (2S,4R)-N-((1H-pyrrolo[3,2- c]pyridin-2-yl)methyl)-4- (difluoromethoxy)-1-((3-p- tolyloxy)benzoyl)glycyl) pyrrolidine-2-carboxamide |
| 273 | 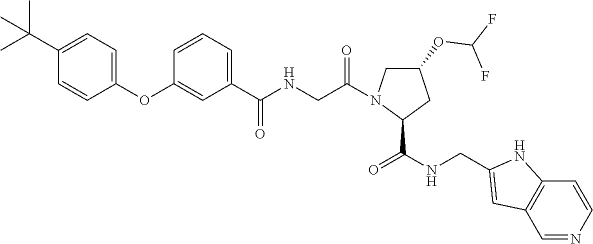 [SMILES: CC(C)(C)c1ccc(Oc2cccc(C(=O)NCC(=O)N3C[C@H](OC(F)F)C[C@H]3C(=O)NCc3cc4cnccc4[nH]3)c2)cc1] | (2S,4R)-N-((1H-pyrrolo[3,2- c]pyridin-2-yl)methyl)-1-((3-(4- (tert- butyl)phenoxy)benzoyl)glycyl)-4- (difluoromethoxy)pyrrolidine-4- carboxamide |
| 274 | 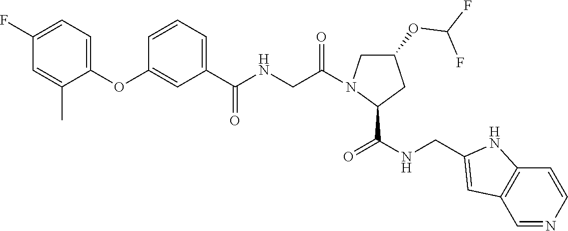 [SMILES: Cc1cc(F)ccc1Oc1cccc(C(=O)NCC(=O)N2C[C@H](OC(F)F)C[C@H]2C(=O)NCc2cc3cnccc3[nH]2)c1] | (2S,4R)-N-((1H-pyrrolo[3,2- c]pyridin-2-yl)methyl)-4- (difluoromethoxy)-1-((3-(4- fluoro-2- methylphenoxy)benzoyl)glycyl) pyrrolidine-2-carboxamide |
| 275 | 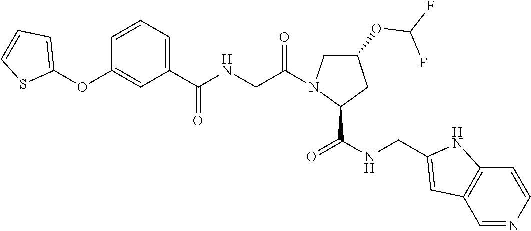 [SMILES: O=C(NCC(=O)N1C[C@H](OC(F)F)C[C@H]1C(=O)NCc1cc2cnccc2[nH]1)c1cccc(Oc2cccs2)c1] | (2S,4R)-N-((1H-pyrrolo[3,2- c]pyridin-2-yl)methyl)-4- (difluoromethoxy)-1-((3- (thiophen-2- yloxy)benzoyl)glycyl)pyrrolidine- 2-carboxamide |
| 276 | 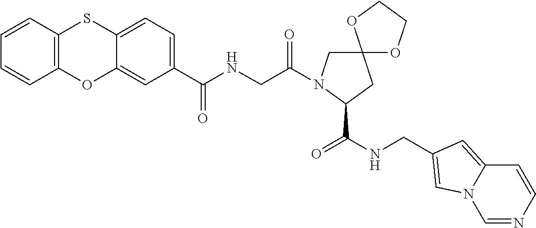 [SMILES: O=C(NCC(=O)N1CC2(C[C@H]1C(=O)NCc1cc3ccncn3c1)OCCO2)c1ccc2c(c1)Oc1ccccc1S2] | (S)-7-((phenoxathiine-3- carbonyl)glycyl)-N-(pyrrolo[1,2- c]pyrimidin-6-ylmethyl)-1,4- dioxa-7-azaspiro[4.4]nonane-8- carboxamide |
| 277 | 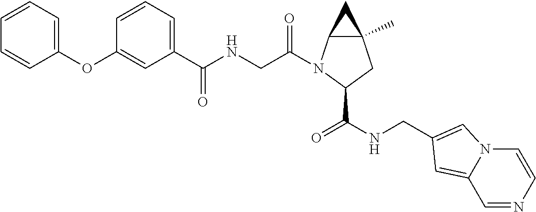 [SMILES: C[C@@]12C[C@@H]1N(C(=O)CNC(=O)c1cccc(Oc3ccccc3)c1)[C@H](C(=O)NCc1cc3cnccn3c1)C2] | (1S,3S,5S)-5-methyl-2-((3- phenoxybenzoyl)glycyl)-N- (pyrrolo[1,2-a]pyrazin-7- ylmethyl)-2- azabicyclo[3.1.0]hexane-3- carboxamide |
| 278 | 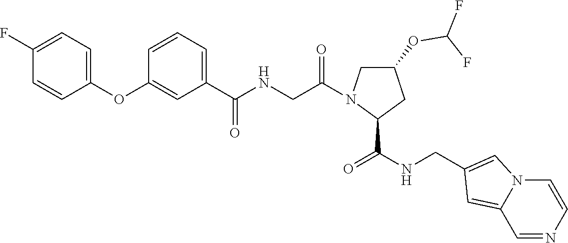 [SMILES: O=C(NCC(=O)N1C[C@H](OC(F)F)C[C@H]1C(=O)NCc1cc2cnccn2c1)c1cccc(Oc2ccc(F)cc2)c1] | (2S,4R)-4-(difluoromethoxy)-1- ((3-(4- fluorophenoxy)benzoyl)glycyl)- N-(pyrrolo[1,2-a]pyrazin-7- ylmethyl)pyrrolidine-2- carboxamide |
| 279 | 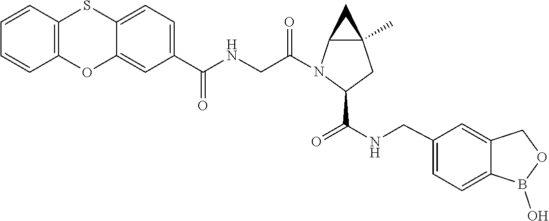 [SMILES: C[C@@]12C[C@@H]1N(C(=O)CNC(=O)c1ccc3c(c1)Oc1ccccc1S3)[C@H](C(=O)NCc1ccc3c(c1)COB3O)C2] | (1S,3S,5S)-N-((1-hydroxy-1,3- dihydrobenzo[c][1,2]oxaborol-5- yl)methyl)-5-methyl-2- ((phenoxathiine-3- carbonyl)glycyl)-2- azabicyclo[3.1.0]hexane-3- carboxamide |
| 280 | 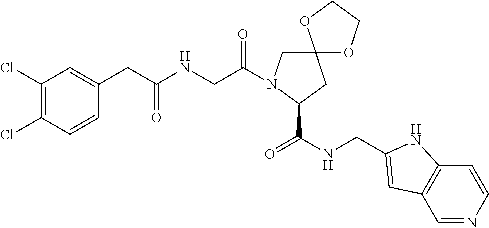 [SMILES: O=C(Cc1ccc(Cl)c(Cl)c1)NCC(=O)N1CC2(C[C@H]1C(=O)NCc1cc3cnccc3[nH]1)OCCO2] | (S)-N-((1H-pyrrolo[3,2-c]pyridin- 2-yl)methyl)-7-((2-(3,4- dichlorophenyl)acetyl)glycyl)-1,4- dioxa-7-azaspiro[4.4]nonane-8- carboxamide |
| 281 | 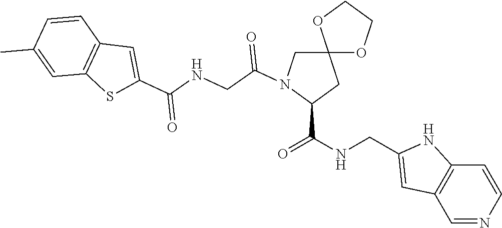 [SMILES: Cc1ccc2cc(C(=O)NCC(=O)N3CC4(C[C@H]3C(=O)NCc3cc5cnccc5[nH]3)OCCO4)sc2c1] | (S)-N-((1H-pyrrolo[3,2-c]pyridin- 2-yl)methyl)-7-((6- methylbenzo[b]thiophene-2- carbonyl)glycyl)-1,4-dioxa-7- azaspiro[4.4]nonane-8- carboxamide |
| 282 | 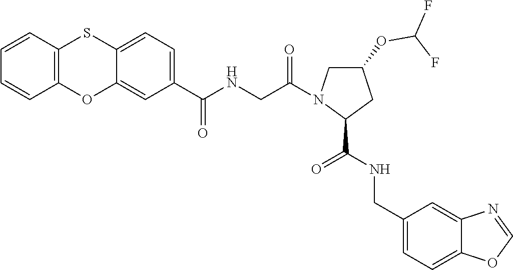 [SMILES: O=C(NCC(=O)N1C[C@H](OC(F)F)C[C@H]1C(=O)NCc1ccc2ocnc2c1)c1ccc2c(c1)Oc1ccccc1S2] | (2S,4R)-N-(benzo[d]oxazol-5- ylmethyl)-4-(difluoromethoxy)-1- ((phenoxathiine-3- carbonyl)glycyl)pyrrolidine-2- carboxamide |
| 283 | 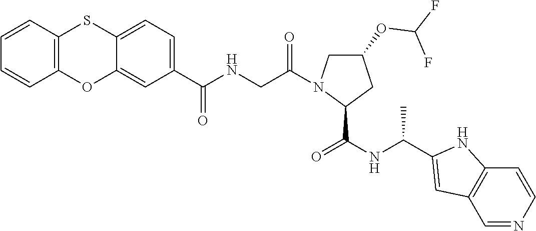 [SMILES: C[C@@H](NC(=O)[C@@H]1C[C@@H](OC(F)F)CN1C(=O)CNC(=O)c1ccc2c(c1)Oc1ccccc1S2)c1cc2cnccc2[nH]1] | N-(2-((2S,4R)-2-(((R)-1-(1H- pyrrolo[3,2-c]pyridin-2- yl)ethyl)carbamoyl)-4- (difluoromethoxy)pyrrolidin-1- yl)-2- oxoethyl)benzo[5,6][1,4]oxathiino [3,2-b]pyridin-3-carboxamide |
| 284 | 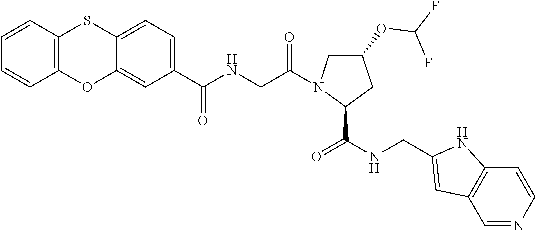 [SMILES: O=C(NCC(=O)N1C[C@H](OC(F)F)C[C@H]1C(=O)NCc1cc2cnccc2[nH]1)c1ccc2c(c1)Oc1ccccc1S2] | N-(2-((2S,4R)-2-(((1H- pyrrolo[3,2-c]pyridin-2- yl)methyl)carbamoyl)-4- (difluoromethoxy)pyrrolidin-1- yl)-2- oxoethyl)benzo[5,6][1,4]oxathiino [3,2-b]pyridine-3-carboxamide |
| 285 | 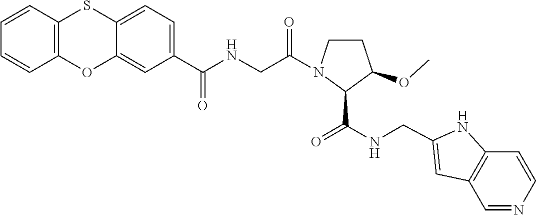 [SMILES: CO[C@@H]1CCN(C(=O)CNC(=O)c2ccc3c(c2)Oc2ccccc2S3)[C@@H]1C(=O)NCc1cc2cnccc2[nH]1] | (2S,3R)-N-((1H-pyrrolo[3,2- c]pyridin-2-yl)methyl)-3- methoxy-1-((phenoxathiine-3- carbonyl)glycyl)pyrrolidine-2- carboxamide |
| 286 | 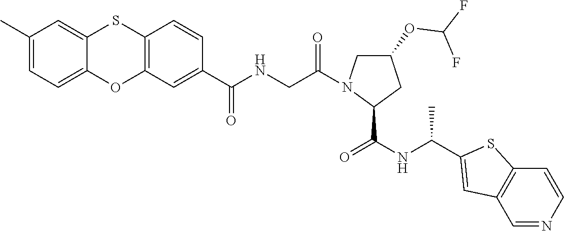 [SMILES: Cc1ccc2c(c1)Sc1ccc(C(=O)NCC(=O)N3C[C@H](OC(F)F)C[C@H]3C(=O)N[C@H](C)c3cc4cnccc4s3)cc1O2] | (2S,4R)-N-((R)-1-(1H- pyrrolo[3,2-c]pyridin-2-yl)ethyl)- 4-(difluoromethoxy)-1-((8- methylphenoxathiine-3- carbonyl)glycyl)pyrrolidine-2- carboxamide |
| 287 | 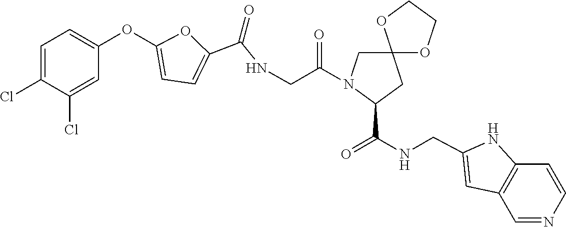 [SMILES: O=C(NCC(=O)N1CC2(C[C@H]1C(=O)NCc1cc3cnccc3[nH]1)OCCO2)c1ccc(Oc2ccc(Cl)c(Cl)c2)o1] | (S)-N-((1H-pyrrolo[3,2-c]pyridin- 2-yl)methyl)-7-((5-(3,4- dichlorophenoxy)furan-2- carbonyl)glycyl)-1,4-dioxa-7- azaspiro[4.4]nonane-8- carboxamide |
| 288 | 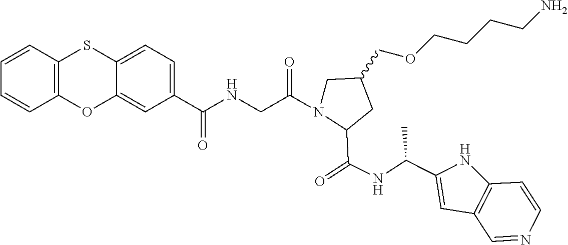 [SMILES: C[C@@H](NC(=O)C1CC(COCCCCN)CN1C(=O)CNC(=O)c1ccc2c(c1)Oc1ccccc1S2)c1cc2cnccc2[nH]1] | (2S)-N-((R)-1-(1H-pyrrolo[3,2- c]pyridin-2-yl)ethyl)-4-((4- aminobutoxy)methyl)-1- ((phenoxathiine-3- carbonyl)glycyl)pyrrolidine-2- carboxamide |
| 289 | 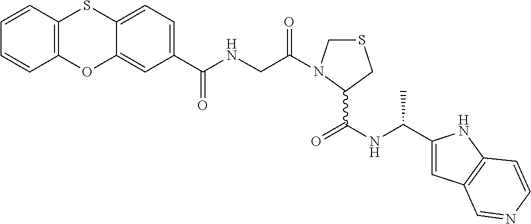 [SMILES: C[C@@H](NC(=O)C1CSCN1C(=O)CNC(=O)c1ccc2c(c1)Oc1ccccc1S2)c1cc2cnccc2[nH]1] | N-((R)-1-(1H-pyrrolo[3,2- c]pyridin-2-yl)ethyl)-3- ((phenoxathiine-3- carbonyl)glycyl)thiazolidine-4- carboxamide |
| 290 | 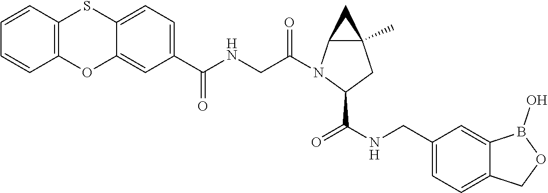 [SMILES: C[C@@]12C[C@@H]1N(C(=O)CNC(=O)c1ccc3c(c1)Oc1ccccc1S3)[C@H](C(=O)NCc1ccc3c(c1)B(O)OC3)C2] | (1S,3S,5S)-N-((1-hydroxy-1,3- dihydrobenzo[c][1,2]oxaborol-6- yl)methyl)-5-methyl-2- ((phenoxathiine-3- carbonyl)glycyl)-2- azabicyclo[3.1.0]hexane-3- carboxamide |
| 291 | 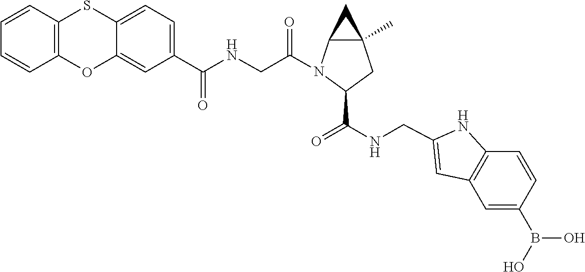 [SMILES: C[C@@]12C[C@@H]1N(C(=O)CNC(=O)c1ccc3c(c1)Oc1ccccc1S3)[C@H](C(=O)NCc1cc3cc(B(O)O)ccc3[nH]1)C2] | (2-(((1S,3S,5S)-5-methyl-2- ((phenoxathiine-3- carbonyl)glycyl)-2- azabicyclo[3.1.0]hexane-3- carboxamido)methyl)-1H-indol-5- yl)boronic acid |
| 292 | 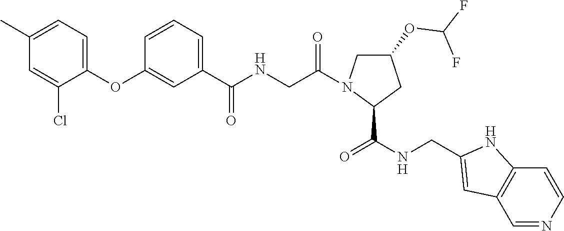 [SMILES: Cc1ccc(Oc2cccc(C(=O)NCC(=O)N3C[C@H](OC(F)F)C[C@H]3C(=O)NCc3cc4cnccc4[nH]3)c2)c(Cl)c1] | (2S,4R)-N-((1H-pyrrolo[3,2- c]pyridin-2-yl)methyl)-1-((3-(2- chloro-4- methylphenoxy)benzoyl)glycyl)- 4-(difluoromethoxy)pyrrolidine-2- carboxamide |
| 293 | 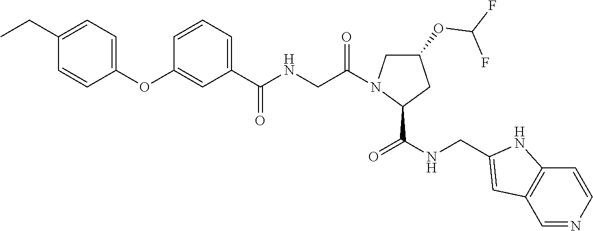 [SMILES: CCc1ccc(Oc2cccc(C(=O)NCC(=O)N3C[C@H](OC(F)F)C[C@H]3C(=O)NCc3cc4cnccc4[nH]3)c2)cc1] | (2S,4R)-N-((1H-pyrrolo[3,2- c]pyridin-2-yl)methyl)-4- (difluoromethoxy)-1-((3-(4- ethylphenoxy)benzoyl)glycyl) pyrrolidine-2-carboxamide |
| 294 | 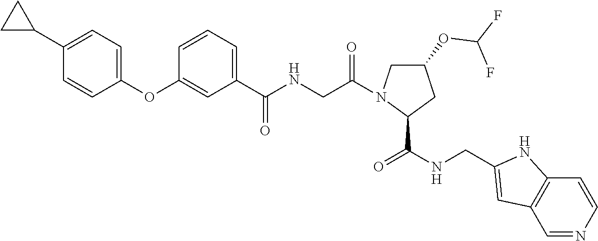 [SMILES: O=C(NCC(=O)N1C[C@H](OC(F)F)C[C@H]1C(=O)NCc1cc2cnccc2[nH]1)c1cccc(Oc2ccc(C3CC3)cc2)c1] | (2S,4R)-N-((1H-pyrrolo[3,2- c]pyridin-2-yl)methyl)-1-((3-(4- cyclopropylphenoxy)benzoyl) glycyl)-4- (difluoromethoxy)pyrrolidine-2- carboxamide |
| 295 | 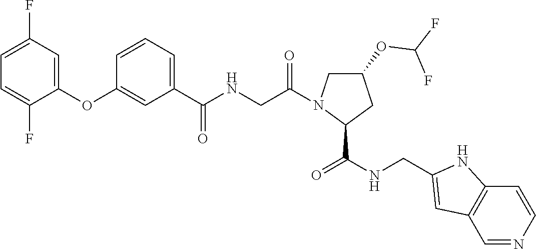 [SMILES: O=C(NCC(=O)N1C[C@H](OC(F)F)C[C@H]1C(=O)NCc1cc2cnccc2[nH]1)c1cccc(Oc2cc(F)ccc2F)c1] | (2S,4R)-N-((1H-pyrrolo[3,2- c]pyridin-2-yl)methyl)-4- (difluoromethoxy)-1-((3-(2,5- difluorophenoxy)benzoyl) glycyl) pyrrolidine-2-carboxamide |
| 296 | 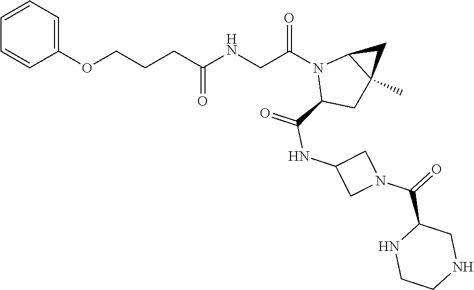 [SMILES: C[C@@]12C[C@@H]1N(C(=O)CNC(=O)CCCOc1ccccc1)[C@H](C(=O)NC1CN(C(=O)[C@H]3CNCCN3)C1)C2] | (1S,3S,5S)-5-methyl-2-((4- phenoxybutanoyl)glycyl)-N-(1- ((R)-piperazine-2- carbonyl)azetidin-3-yl)-2- azabicyclo[3.1.0]hexane-3- carboxamide |
| 297 | 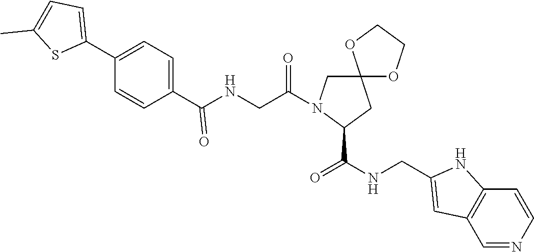 [SMILES: Cc1ccc(-c2ccc(C(=O)NCC(=O)N3CC4(C[C@H]3C(=O)NCc3cc5cnccc5[nH]3)OCCO4)cc2)s1] | (S)-N-((1H-pyrrolo[3,2-c]pyridin- 2-yl)methyl)-7-((4-(5- methylthiophen-2- yl)benzoyl)glycyl)-1,4-dioxa-7- azaspiro[4.4]nonane-8- carboxamide |
| 298 | 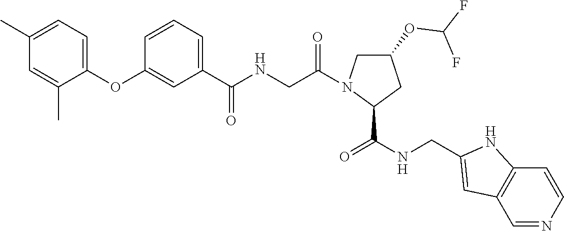 [SMILES: Cc1ccc(Oc2cccc(C(=O)NCC(=O)N3C[C@H](OC(F)F)C[C@H]3C(=O)NCc3cc4cnccc4[nH]3)c2)c(C)c1] | (2S,4R)-N-((1H-pyrrolo[3,2- c]pyridin-2-yl)methyl)-4- (difluoromethoxy)-1-((3-(2,4- dimethylphenoxy)benzoyl)glycyl) pyrrolidine-2-carboxamide |
| 299 | 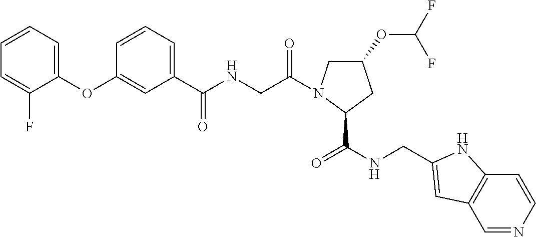 [SMILES: O=C(NCC(=O)N1C[C@H](OC(F)F)C[C@H]1C(=O)NCc1cc2cnccc2[nH]1)c1cccc(Oc2ccccc2F)c1] | (2S,4R)-N-((1H-pyrrolo[3,2- c]pyridin-2-yl)methyl)-4- (difluoromethoxy)-1-((3-(2- fluorophenoxy)benzoyl)glycyl) pyrrolidine-2-carboxamide |
| 300 | 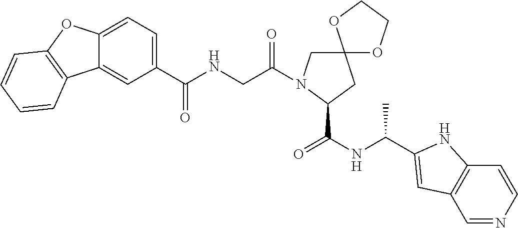 [SMILES: C[C@@H](NC(=O)[C@@H]1CC2(CN1C(=O)CNC(=O)c1ccc3oc4ccccc4c3c1)OCCO2)c1cc2cnccc2[nH]1] | (S)-N-((R)-1-(1H-pyrrolo[3,2- c]pyridin-2-yl)ethyl)-7- ((dibenzo[b,d]furan-2- carbonyl)glycyl)-1,4-dioxa-7- azaspiro[4.4]nonane-8- carboxamide |
| 301 | 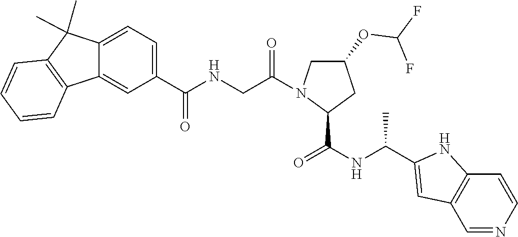 [SMILES: C[C@@H](NC(=O)[C@@H]1C[C@@H](OC(F)F)CN1C(=O)CNC(=O)c1ccc2c(c1)-c1ccccc1C2(C)C)c1cc2cnccc2[nH]1] | (2S,4R)-N-((1H-pyrrolo[3,2- c]pyridin-2-yl)methyl)-4- (difluoromethoxy)-1-((9,9- dimethyl-9H-fluorene-3- carbonyl)glycyl) pyrrolidine-2-carboxamide |
| 302 | 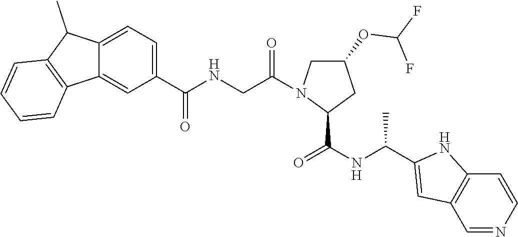 [SMILES: CC1c2ccccc2-c2cc(C(=O)NCC(=O)N3C[C@H](OC(F)F)C[C@H]3C(=O)N[C@H](C)c3cc4cnccc4[nH]3)ccc21] | (2S,4R)-N-((1H-pyrrolo[3,2- c]pyridin-2-yl)methyl)-4- (difluoromethoxy)-1-((9-methyl- 9H-fluorene-3- carbonyl)glycyl) pyrrolidine-2-carboxamide |
| 303 | 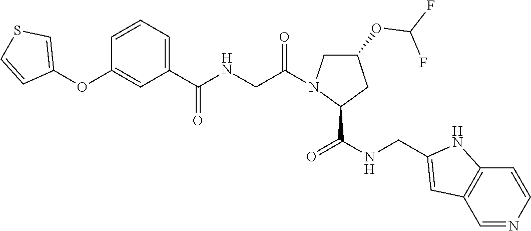 [SMILES: O=C(NCC(=O)N1C[C@H](OC(F)F)C[C@H]1C(=O)NCc1cc2cnccc2[nH]1)c1cccc(Oc2ccsc2)c1] | (2S,4R)-N-((1H-pyrrolo[3,2- c]pyridin-2-yl)methyl)-4- (difluoromethoxy)-1-((3- (thiophen-3- yloxy)benzoyl)glycyl) pyrrolidine-2-carboxamide |
| 304 | 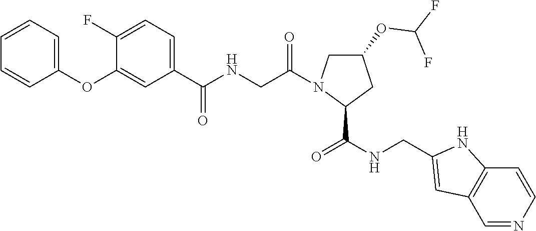 [SMILES: O=C(NCC(=O)N1C[C@H](OC(F)F)C[C@H]1C(=O)NCc1cc2cnccc2[nH]1)c1ccc(F)c(Oc2ccccc2)c1] | (2S,4R)-N-((1H-pyrrolo[3,2- c]pyridin-2-yl)methyl)-4- (difluoromethoxy)-1-((4-fluoro-3- phenoxybenzoyl)glycyl) pyrrolidine-2-carboxamide |
| 305 | 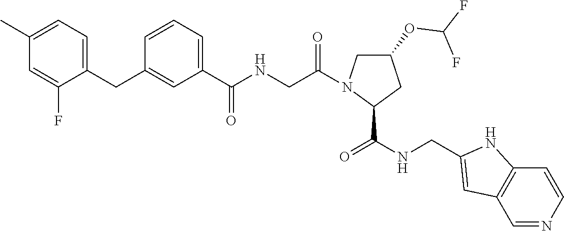 [SMILES: Cc1ccc(Cc2cccc(C(=O)NCC(=O)N3C[C@H](OC(F)F)C[C@H]3C(=O)NCc3cc4cnccc4[nH]3)c2)c(F)c1] | (2S,4R)-N-((1H-pyrrolo[3,2- c]pyridin-2-yl)methyl)-4- (difluoromethoxy)-1-((3-(2- fluoro-4- methylbenzyl)benzoyl)glycyl) pyrrolidine-2-carboxamide |
| 306 | 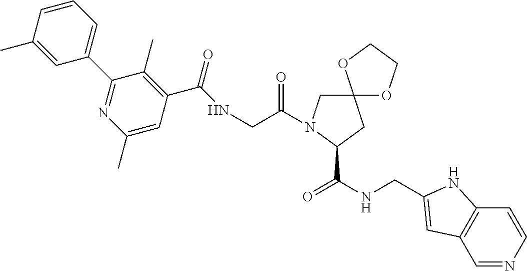 [SMILES: Cc1cccc(-c2nc(C)cc(C(=O)NCC(=O)N3CC4(C[C@H]3C(=O)NCc3cc5cnccc5[nH]3)OCCO4)c2C)c1] | (S)-N-((1H-pyrrolo[3,2-c]pyridin- 2-yl)methyl)-7-((3,6-dimethyl-2- (m-tolyl)isonicotinoyl)glycyl)- 1,4-dioxa-7-azaspiro[4.4]nonane- 8-carboxamide |
| 307 | 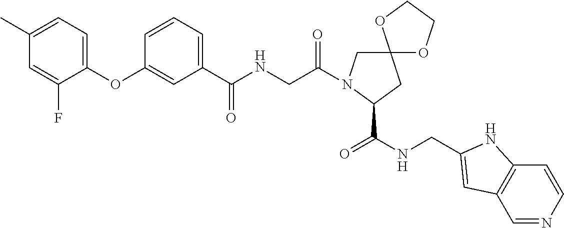 [SMILES: Cc1ccc(Oc2cccc(C(=O)NCC(=O)N3CC4(C[C@H]3C(=O)NCc3cc5cnccc5[nH]3)OCCO4)c2)c(F)c1] | (S)-N-((1H-pyrrolo[3,2-c]pyridin- 2-yl)methyl)-7-((3-(2-fluoro-4- methylphenoxy)benzoyl)glycyl)- 1,4-dioxa-7-azaspiro[4.4]nonane- 8-carboxamide |
| 308 | 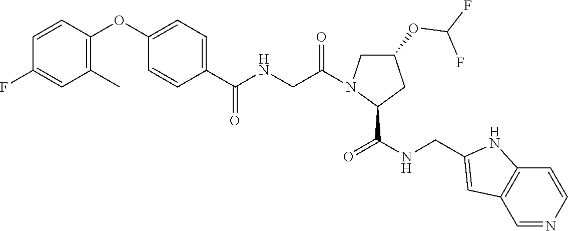 [SMILES: Cc1cc(F)ccc1Oc1ccc(C(=O)NCC(=O)N2C[C@H](OC(F)F)C[C@H]2C(=O)NCc2cc3cnccc3[nH]2)cc1] | (2S,4R)-N-((1H-pyrrolo[3,2- c]pyridin-2-yl)methyl)-4- (difluoromethoxy)-1-((4-(4- fluoro-2- methylphenoxy)benzoyl)glycyl) pyrrolidine-2-carboxamide |
| 309 | 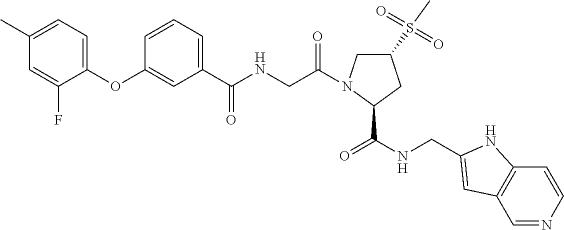 [SMILES: Cc1ccc(Oc2cccc(C(=O)NCC(=O)N3C[C@H](S(C)(=O)=O)C[C@H]3C(=O)NCc3cc4cnccc4[nH]3)c2)c(F)c1] | (2S,4R)-N-((1H-pyrrolo[3,2- c]pyridin-2-yl)methyl)-1-((3-(2- fluoro-4- methylphenoxy)benzoyl)glycyl)- 4-(methylsulfonyl) pyrrolidine-2-carboxamide |
| 310 | 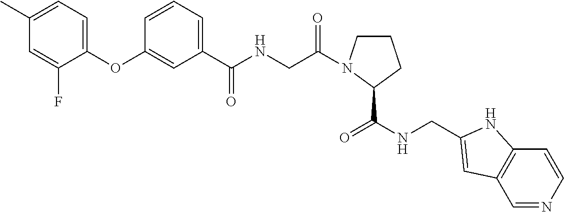 [SMILES: Cc1ccc(Oc2cccc(C(=O)NCC(=O)N3CCC[C@H]3C(=O)NCc3cc4cnccc4[nH]3)c2)c(F)c1] | (S)-N-((1H-pyrrolo[3,2-c]pyridin- 2-yl)methyl)-1-((3-(2-fluoro-4- methylphenoxy)benzoyl)glycyl) pyrrolidine-2-carboxamide |
| 311 | 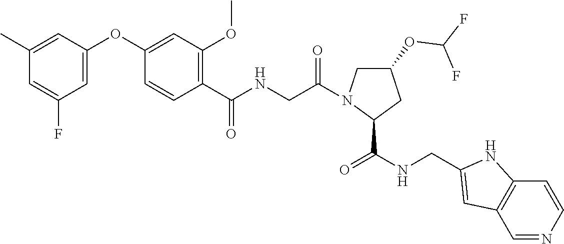 [SMILES: COc1cc(Oc2cc(C)cc(F)c2)ccc1C(=O)NCC(=O)N1C[C@H](OC(F)F)C[C@H]1C(=O)NCc1cc2cnccc2[nH]1] | (2S,4R)-N-((1H-pyrrolo[3,2- c]pyridin-2-yl)methyl)-4- (difluoromethoxy)-1-((5-(2- fluoro-4-methylphenoxy)-2- methoxybenzoyl)glycyl) pyrrolidine-2-carboxamide |
| 312 | 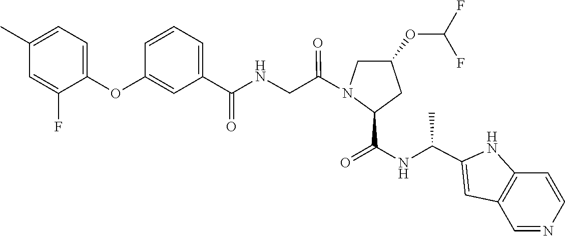 [SMILES: Cc1ccc(Oc2cccc(C(=O)NCC(=O)N3C[C@H](OC(F)F)C[C@H]3C(=O)N[C@H](C)c3cc4cnccc4[nH]3)c2)c(F)c1] | (2S,4R)-N-((R)-1-(1H- pyrrolo[3,2-c]pyridin-2-yl)ethyl)- 4-(difluoromethoxy)-1-((3-(2- fluoro-4- methylphenoxy)benzoyl)glycyl) pyrrolidine-2-carboxamide |
| 313 | 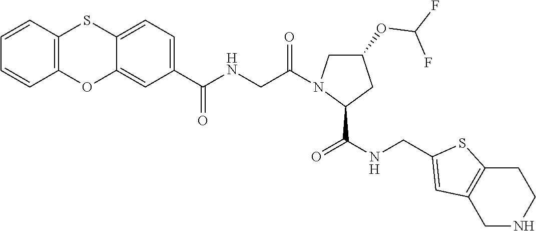 [SMILES: O=C(NCC(=O)N1C[C@H](OC(F)F)C[C@H]1C(=O)NCc1cc2c(s1)CCNC2)c1ccc2c(c1)Oc1ccccc1S2] | (2S,4R)-4-(difluoromethoxy)-1- ((phenoxathiine-3- carbonyl)glycyl)-N-((4,5,6,7- tetrahydrothieno[3,2-c]pyridin-2- yl)methyl)pyrrolidine-2- carboxamide |
| 314 | 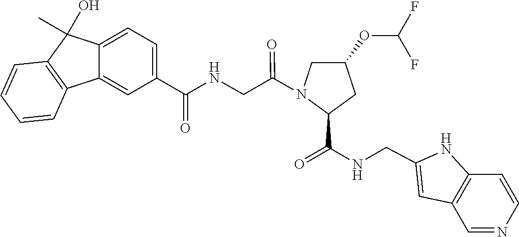 [SMILES: CC1(O)c2ccccc2-c2cc(C(=O)NCC(=O)N3C[C@H](OC(F)F)C[C@H]3C(=O)NCc3cc4cnccc4[nH]3)ccc21] | (2S,4R)-N-((1H-pyrrolo[3,2- c]pyridin-2-yl)methyl)-4- (difluoromethoxy)-1-((9-hydroxy- 9-methyl-9H-fluorene-3- carbonyl)glycyl) pyrrolidine-2-carboxamide |
| 315 | 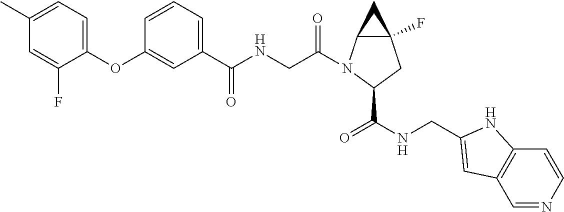 [SMILES: Cc1ccc(Oc2cccc(C(=O)NCC(=O)N3[C@H]4C[C@@]4(F)C[C@H]3C(=O)NCc3cc4cnccc4[nH]3)c2)c(F)c1] | (1S,3S,5S)-N-((1H-pyrrolo[3,2- c]pyridin-2-yl)methyl)-2-((3-(2- fluoro-4- methylphenoxy)benzoyl)glycyl)- 5-methyl-2- azabicyclo[3.1.0]hexane-3- carboxamide |
| 316 | 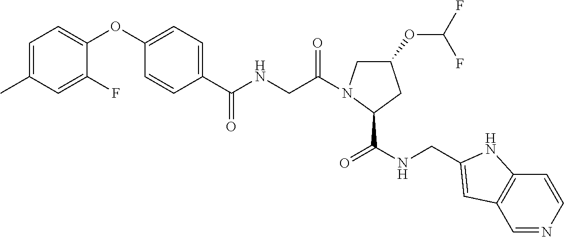 [SMILES: Cc1ccc(Oc2ccc(C(=O)NCC(=O)N3C[C@H](OC(F)F)C[C@H]3C(=O)NCc3cc4cnccc4[nH]3)cc2)c(F)c1] | (2S,4R)-N-((1H-pyrrolo[3,2- c]pyridin-2-yl)methyl)-4- (difluoromethoxy)-1-((4-(2- fluoro-4- methylphenoxy)benzoyl)glycyl) pyrrolidine-2-carboxamide |
| 317 | 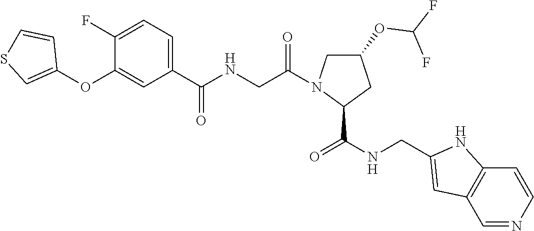 [SMILES: O=C(NCC(=O)N1C[C@H](OC(F)F)C[C@H]1C(=O)NCc1cc2cnccc2[nH]1)c1ccc(F)c(Oc2ccsc2)c1] | (2S,4R)-N-((1H-pyrrolo[3,2- c]pyridin-2-yl)methyl)-4- (difluoromethoxy)-1-((4-fluoro-3- (thiophen-3- yloxy)benzoyl)glycyl) pyrrolidine-2-carboxamide |
| 318 | 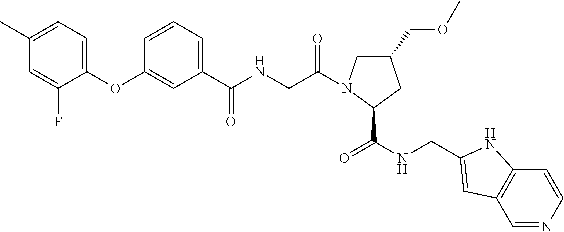 [SMILES: COC[C@@H]1C[C@@H](C(=O)NCc2cc3cnccc3[nH]2)N(C(=O)CNC(=O)c2cccc(Oc3ccc(C)cc3F)c2)C1] | (2S,4R)-N-((1H-pyrrolo[3,2- c]pyridin-2-yl)methyl)-1-((3-(2- fluoro-4- methylphenoxy)benzoyl)glycyl)- 4-(methoxymethyl) pyrrolidine-2-carboxamide |
| 319 | 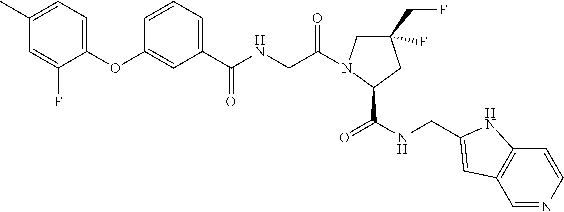 [SMILES: Cc1ccc(Oc2cccc(C(=O)NCC(=O)N3C[C@@](F)(CF)C[C@H]3C(=O)NCc3cc4cnccc4[nH]3)c2)c(F)c1] | (2S,4R)-N-((1H-pyrrolo[3,2- c]pyridin-2-yl)methyl)-4-fluoro-1- ((3-(2-fluoro-4- methylphenoxy)benzoyl)glycyl)- 4-(fluoromethyl) pyrrolidine-2-carboxamide |
| 320 | 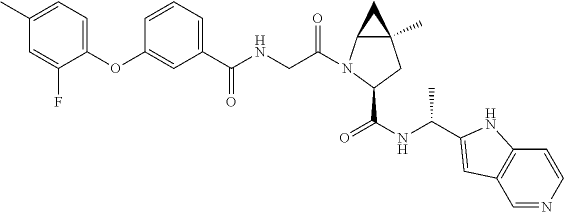 [SMILES: Cc1ccc(Oc2cccc(C(=O)NCC(=O)N3[C@H]4C[C@@]4(C)C[C@H]3C(=O)N[C@H](C)c3cc4cnccc4[nH]3)c2)c(F)c1] | (1S,3S,5S)-N-((R)-1-(1H- pyrrolo[3,2-c]pyridin-2-yl)ethyl)- 2-((3-(2-fluoro-4- methylphenoxy)benzoyl)glycyl)- 5-methyl-2- azabicyclo[3.1.0]hexane-3- carboxamide |
| 321 | 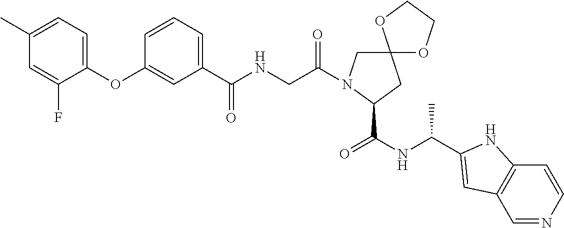 [SMILES: Cc1ccc(Oc2cccc(C(=O)NCC(=O)N3CC4(C[C@H]3C(=O)N[C@H](C)c3cc5cnccc5[nH]3)OCCO4)c2)c(F)c1] | (S)-N-((R)-1-(1H-pyrrolo[3,2- c]pyridin-2-yl)ethyl)-7-((3-(2- fluoro-4- methylphenoxy)benzoyl)glycyl)- 1,4-dioxa-7-azaspiro[4.4]nonane- 8-carboxamide |
| 322 | 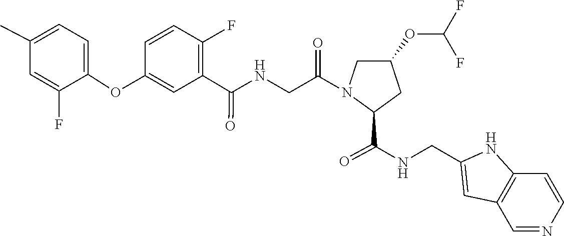 [SMILES: Cc1ccc(Oc2ccc(F)c(C(=O)NCC(=O)N3C[C@H](OC(F)F)C[C@H]3C(=O)NCc3cc4cnccc4[nH]3)c2)c(F)c1] | (2S,4R)-N-((1H-pyrrolo[3,2- c]pyridin-2-yl)methyl)-4- (difluoromethoxy)-1-((2-fluoro-5- (2-fluoro-4- methylphenoxy)benzoyl)glycyl) pyrrolidine-2-carboxamide |
| 323 | 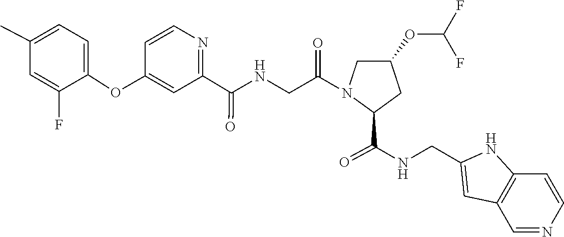 [SMILES: Cc1ccc(Oc2ccnc(C(=O)NCC(=O)N3C[C@H](OC(F)F)C[C@H]3C(=O)NCc3cc4cnccc4[nH]3)c2)c(F)c1] | N-(2-((2S,4R)-2-(((1H- pyrrolo[3,2-c]pyridin-2- yl)methyl)carbamoyl)-4- (difluoromethoxy)pyrrolidin-1- yl)-2-oxoethyl)-4-(2-fluoro-4- methylphenoxy)picolinamide |
| 324 | 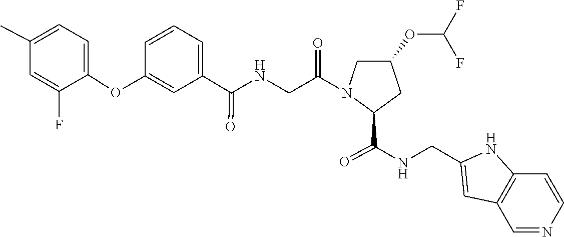 [SMILES: Cc1ccc(Oc2cccc(C(=O)NCC(=O)N3C[C@H](OC(F)F)C[C@H]3C(=O)NCc3cc4cnccc4[nH]3)c2)c(F)c1] | (2S,4R)-N-((1H-pyrrolo[3,2- c]pyridin-2-yl)methyl)-4- (difluoromethoxy)-1-((3-((2- fluoro-4- methylphenyl)amino)benzoyl) glycyl)pyrrolidine-2-carboxamide |
| 325 | 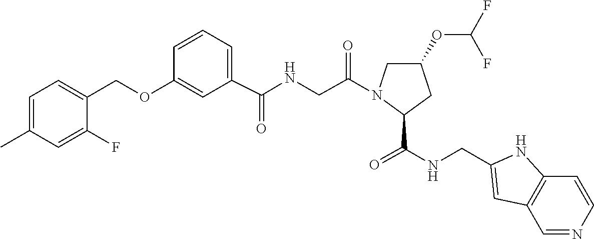 [SMILES: Cc1ccc(COc2cccc(C(=O)NCC(=O)N3C[C@H](OC(F)F)C[C@H]3C(=O)NCc3cc4cnccc4[nH]3)c2)c(F)c1] | (2S,4R)-N-((1H-pyrrolo[3,2- c]pyridin-2-yl)methyl)-4- (difluoromethoxy)-1-((3-((2- fluoro-4- methylbenzyl)oxy)benzoyl)glycyl) pyrrolidine-2-carboxamide |
| 326 | 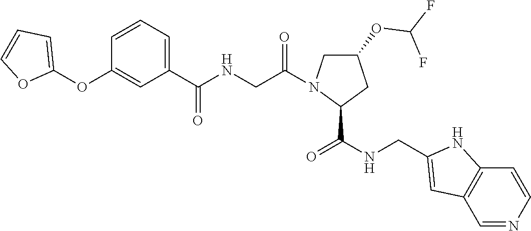 [SMILES: O=C(NCC(=O)N1C[C@H](OC(F)F)C[C@H]1C(=O)NCc1cc2cnccc2[nH]1)c1cccc(Oc2ccco2)c1] | (2S,4R)-N-((1H-pyrrolo[3,2- c]pyridin-2-yl)methyl)-4- (difluoromethoxy)-1-((3-(furan-2- yloxy)benzoyl)glycyl) pyrrolidine-2-carboxamide |
| 327 | 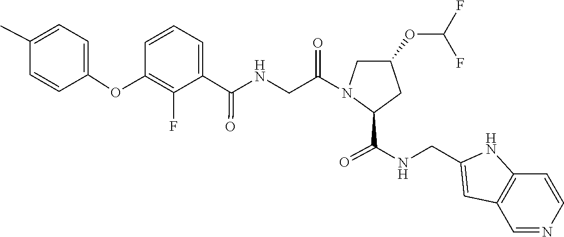 [SMILES: Cc1ccc(Oc2cccc(C(=O)NCC(=O)N3C[C@H](OC(F)F)C[C@H]3C(=O)NCc3cc4cnccc4[nH]3)c2F)cc1] | (2S,4R)-N-((1H-pyrrolo[3,2- c]pyridin-2-yl)methyl)-4- (difluoromethoxy)-1-((2-fluoro-3- (p-tolyloxy)benzoyl)glycyl) pyrrolidine-2-carboxamide |
| 328 | 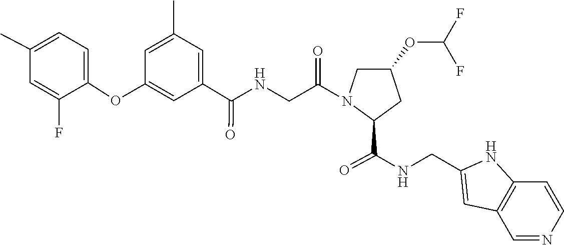 [SMILES: Cc1cc(Oc2ccc(C)cc2F)cc(C(=O)NCC(=O)N2C[C@H](OC(F)F)C[C@H]2C(=O)NCc2cc3cnccc3[nH]2)c1] | (2S,4R)-N-((1H-pyrrolo[3,2- c]pyridin-2-yl)methyl)-4- (difluoromethoxy)-1-((3-(2- fluoro-4-methylphenoxy)-5- methylbenzoyl)glycyl)pyrrolidine- 2-carboxamide |
| 329 | 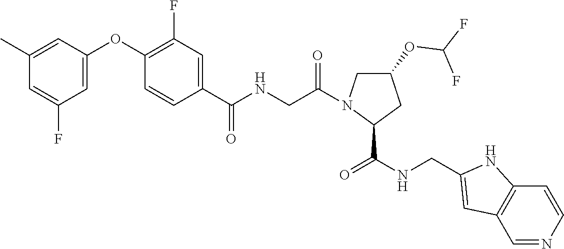 [SMILES: Cc1cc(F)cc(Oc2ccc(C(=O)NCC(=O)N3C[C@H](OC(F)F)C[C@H]3C(=O)NCc3cc4cnccc4[nH]3)cc2F)c1] | (2S,4R)-N-((1H-pyrrolo[3,2- c]pyridin-2-yl)methyl)-4- (difluoromethoxy)-1-((3-fluoro-5- (2-fluoro-4- methylphenoxy)benzoyl)glycyl) pyrrolidine-2-carboxamide |
| 330 | 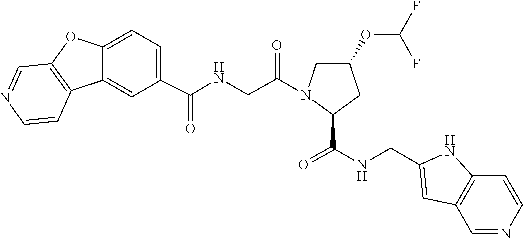 [SMILES: O=C(NCC(=O)N1C[C@H](OC(F)F)C[C@H]1C(=O)NCc1cc2cnccc2[nH]1)c1ccc2oc3cnccc3c2c1] | N-(2-((2S,4R)-2-(((1H- pyrrolo[3,2-c]pyridin-2- yl)methyl)carbamoyl)-4- (difluoromethoxy)pyrrolidin-1- yl)-2-oxoethyl)benzofuro[2,3- c]pyridine-6-carboxamide |
| 331 | 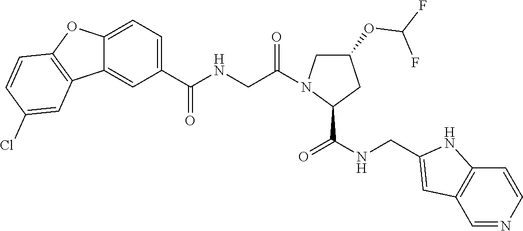 [SMILES: O=C(NCC(=O)N1C[C@H](OC(F)F)C[C@H]1C(=O)NCc1cc2cnccc2[nH]1)c1ccc2oc3ccc(Cl)cc3c2c1] | (2S,4R)-N-((1H-pyrrolo[3,2- c]pyridin-2-yl)methyl)-1-((8- chlorodibenzo[b,d]furan-2- carbonyl)glycyl)-4- (difluoromethoxy) pyrrolidine-2-carboxamide |
| 332 | 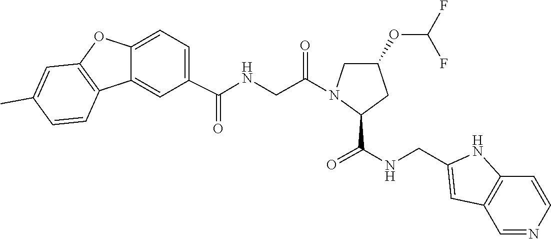 [SMILES: Cc1ccc2c(c1)oc1ccc(C(=O)NCC(=O)N3C[C@H](OC(F)F)C[C@H]3C(=O)NCc3cc4cnccc4[nH]3)cc12] | (2S,4R)-N-((1H-pyrrolo[3,2- c]pyridin-2-yl)methyl)-4- (difluoromethoxy)-1-((7- methyldibenzo[b,d]furan-2- carbonyl)glycyl) pyrrolidine-2-carboxamide |
| 333 | 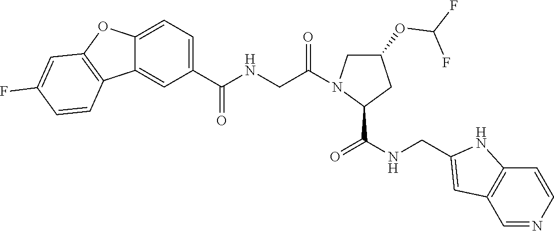 [SMILES: O=C(NCC(=O)N1C[C@H](OC(F)F)C[C@H]1C(=O)NCc1cc2cnccc2[nH]1)c1ccc2oc3cc(F)ccc3c2c1] | (2S,4R)-N-((1H-pyrrolo[3,2- c]pyridin-2-yl)methyl)-4- (difluoromethoxy)-1-((7- fluorodibenzo[b,d]furan-2- carbonyl)glycyl) pyrrolidine-2-carboxamide |
| 334 | 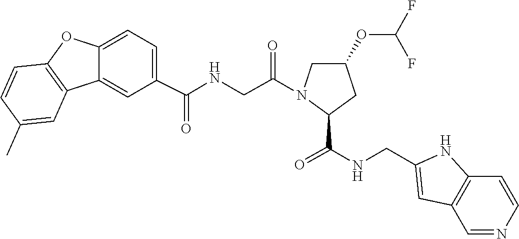 [SMILES: Cc1ccc2oc3ccc(C(=O)NCC(=O)N4C[C@H](OC(F)F)C[C@H]4C(=O)NCc4cc5cnccc5[nH]4)cc3c2c1] | (2S,4R)-N-((1H-pyrrolo[3,2- c]pyridin-2-yl)methyl)-4- (difluoromethoxy)-1-((8- methyldibenzo[b,d]furan-2- carbonyl)glycyl) pyrrolidine-2-carboxamide |
| 335 | 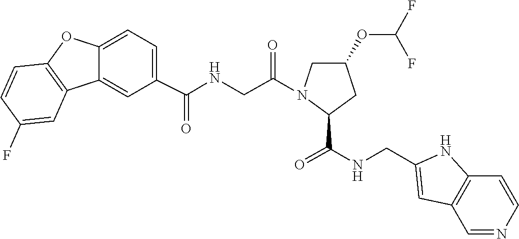 [SMILES: O=C(NCC(=O)N1C[C@H](OC(F)F)C[C@H]1C(=O)NCc1cc2cnccc2[nH]1)c1ccc2oc3ccc(F)cc3c2c1] | (2S,4R)-N-((1H-pyrrolo[3,2- c]pyridin-2-yl)methyl)-4- (difluoromethoxy)-1-((8- fluorodibenzo[b,d]furan-2- carbonyl)glycyl) pyrrolidine-2-carboxamide |
| 336 | 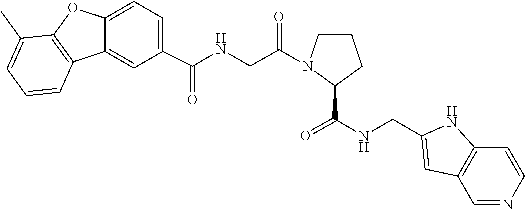 [SMILES: Cc1cccc2c1oc1ccc(C(=O)NCC(=O)N3CCC[C@H]3C(=O)NCc3cc4cnccc4[nH]3)cc12] | (S)-N-((1H-pyrrolo[3,2-c]pyridin- 2-yl)methyl)-1-((6- methyldibenzo[b,d]furan-2- carbonyl)glycyl) pyrrolidine-2-carboxamide |
| 337 | 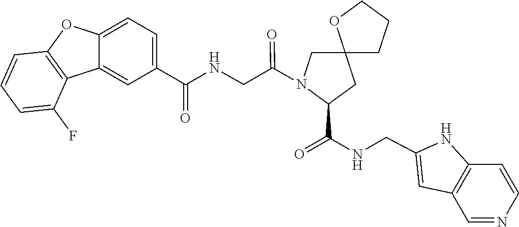 [SMILES: O=C(NCC(=O)N1CC2(CCCO2)C[C@H]1C(=O)NCc1cc2cnccc2[nH]1)c1ccc2oc3cccc(F)c3c2c1] | (8S)-N-((1H-pyrrolo[3,2- c]pyridin-2-yl)methyl)-7-((9- fluorodibenzo[b,d]furan-2- carbonyl)glycyl)-1-oxa-7- azaspiro[4.4]nonane-8- carboxamide |
| 338 | 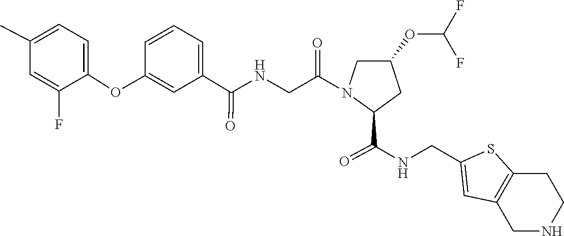 [SMILES: Cc1ccc(Oc2cccc(C(=O)NCC(=O)N3C[C@H](OC(F)F)C[C@H]3C(=O)NCc3cc4c(s3)CCNC4)c2)c(F)c1] | (2S,4R)-4-(difluoromethoxy)-1- ((3-(2-fluoro-4- methylphenoxy)benzoyl)glycyl)- N-((4,5,6,7-tetrahydrothieno[3,2- c]pyridin-2-yl)methyl) pyrrolidine-2-carboxamide |
| 339 | 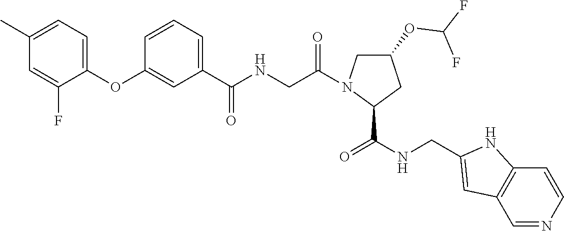 [SMILES: Cc1ccc(Oc2cccc(C(=O)NCC(=O)N3C[C@H](OC(F)F)C[C@H]3C(=O)NCc3cc4cnccc4[nH]3)c2)c(F)c1] | (2S,4R)-4-(difluoromethoxy)-1- ((3-(2-fluoro-4- methylphenoxy)benzoyl)glycyl)- N-((4,5,6,7-tetrahydrothieno[2,3- c]pyridin-2-yl)methyl) pyrrolidine-2-carboxamide |
| 340 | 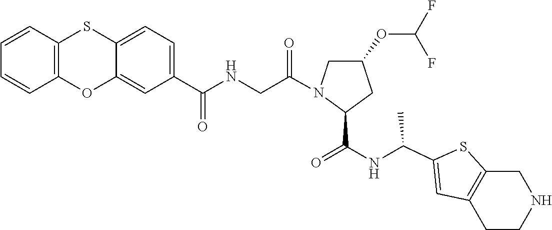 [SMILES: C[C@@H](NC(=O)[C@@H]1C[C@@H](OC(F)F)CN1C(=O)CNC(=O)c1ccc2c(c1)Oc1ccccc1S2)c1cc2c(s1)CNCC2] | (2S,4R)-4-(difluoromethoxy)-1- ((phenoxathiine-3- carbonyl)glycyl)-N-((R)-1- (4,5,6,7-tetrahydrothieno[2,3- c]pyridin-2-yl)ethyl) pyrrolidine-2-carboxamide |
| 341 | 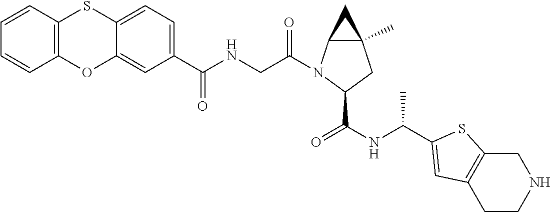 [SMILES: C[C@@H](NC(=O)[C@@H]1C[C@]2(C)C[C@@H]2N1C(=O)CNC(=O)c1ccc2c(c1)Oc1ccccc1S2)c1cc2c(s1)CNCC2] | (1S,3S,5S)-5-methyl-2- ((phenoxathiine-3- carbonyl)glycyl)-N-((R)-1- (4,5,6,7-tetrahydrothieno[2,3- c]pyridin-2-yl)ethyl)-2- azabicyclo[3.1.0]hexane-3- carboxamide |
| 342 | 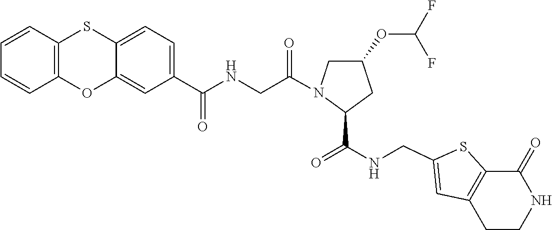 [SMILES: O=C(NCC(=O)N1C[C@H](OC(F)F)C[C@H]1C(=O)NCc1cc2c(s1)C(=O)NCC2)c1ccc2c(c1)Oc1ccccc1S2] | (2S,4R)-4-(difluoromethoxy)-N- ((7-oxo-4,5,6,7- tetrahydrothieno[2,3-c]pyridin-2- yl)methyl)-1-((phenoxathiine-3- carbonyl)glycyl) pyrrolidine-2-carboxamide |
| 343 | 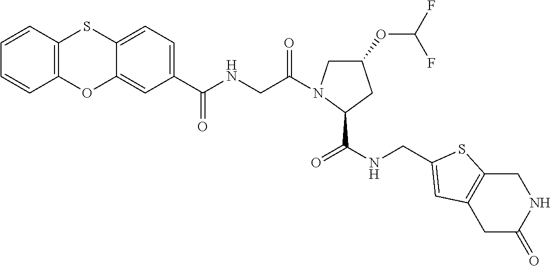 [SMILES: O=C1Cc2cc(CNC(=O)[C@@H]3C[C@@H](OC(F)F)CN3C(=O)CNC(=O)c3ccc4c(c3)Oc3ccccc3S4)sc2CN1] | (2S,4R)-4-(difluoromethoxy)-N- ((5-oxo-4,5,6,7- tetrahydrothieno[2,3-c]pyridin-2- yl)methyl)-1-((phenoxathiine-3- carbonyl)glycyl) pyrrolidine-2-carboxamide |
| 344 | 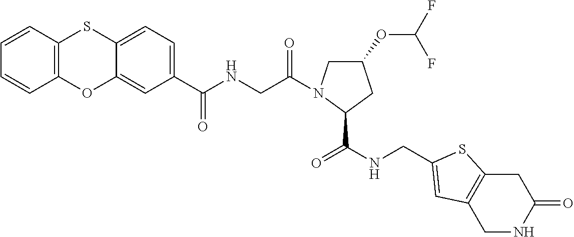 [SMILES: O=C1Cc2sc(CNC(=O)[C@@H]3C[C@@H](OC(F)F)CN3C(=O)CNC(=O)c3ccc4c(c3)Oc3ccccc3S4)cc2CN1] | (2S,4R)-4-(difluoromethoxy)-N- ((6-oxo-4,5,6,7- tetrahydrothieno[3,2-c]pyridin-2- yl)methyl)-1-((phenoxathiine-3- carbonyl)glycyl) pyrrolidine-2-carboxamide |
| 345 | 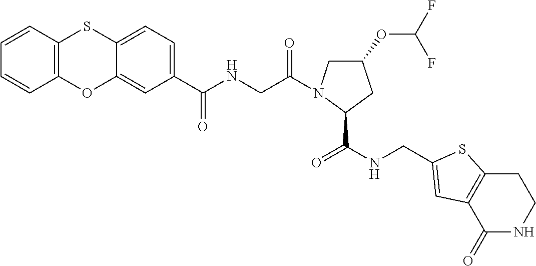 [SMILES: O=C(NCC(=O)N1C[C@H](OC(F)F)C[C@H]1C(=O)NCc1cc2c(s1)CCNC2=O)c1ccc2c(c1)Oc1ccccc1S2] | (2S,4R)-4-(difluoromethoxy)-N- ((4-oxo-4,5,6,7- tetrahydrothieno[3,2-c]pyridin-2- yl)methyl)-1-((phenoxathiine-3- carbonyl)glycyl)pyrrolidine-2- carboxamide |
| 346 | 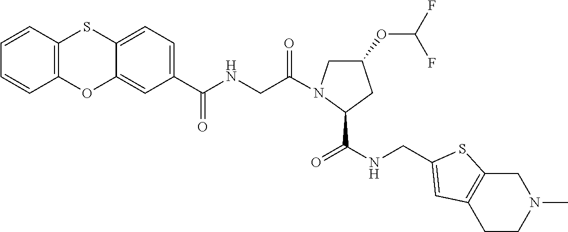 [SMILES: CN1CCc2cc(CNC(=O)[C@@H]3C[C@@H](OC(F)F)CN3C(=O)CNC(=O)c3ccc4c(c3)Oc3ccccc3S4)sc2C1] | (2S,4R)-4-(difluoromethoxy)-N- ((6-methyl-4,5,6,7- tetrahydrothieno[2,3-c]pyridin-2- yl)methyl)-1-((phenoxathiine-3- carbonyl)glycyl) pyrrolidine-2-carboxamide |
| 347 | 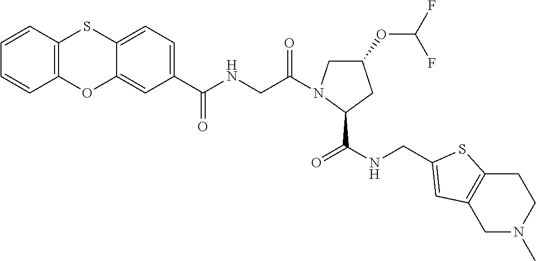 [SMILES: CN1CCc2sc(CNC(=O)[C@@H]3C[C@@H](OC(F)F)CN3C(=O)CNC(=O)c3ccc4c(c3)Oc3ccccc3S4)cc2C1] | (2S,4R)-4-(difluoromethoxy)-N- ((5-methyl-4,5,6,7- tetrahydrothieno[3,2-c]pyridin-2- yl)methyl)-1-((phenoxathiine-3- carbonyl)glycyl) pyrrolidine-2-carboxamide |
| 348 | 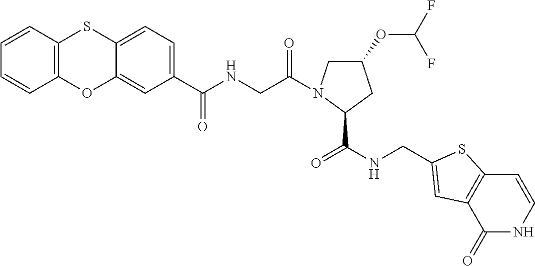 [SMILES: O=C(NCC(=O)N1C[C@H](OC(F)F)C[C@H]1C(=O)NCc1cc2c(=O)[nH]ccc2s1)c1ccc2c(c1)Oc1ccccc1S2] | (2S,4R)-4-(difluoromethoxy)-N- ((4-oxo-4,5-dihydrothieno[3,2- c]pyridin-2-yl)methyl)-1- ((phenoxathiine-3- carbonyl)glycyl) pyrrolidine-2-carboxamide |
| 349 | 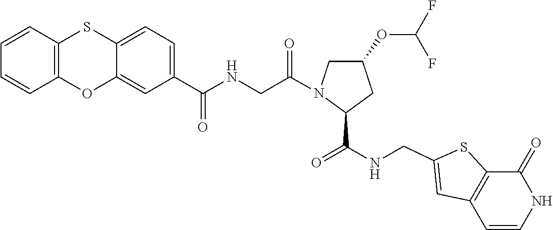 [SMILES: O=C(NCC(=O)N1C[C@H](OC(F)F)C[C@H]1C(=O)NCc1cc2cc[nH]c(=O)c2s1)c1ccc2c(c1)Oc1ccccc1S2] | (2S,4R)-4-(difluoromethoxy)-N- ((7-oxo-6,7-dihydrothieno[2,3- c]pyridin-2-yl)methyl)-1- ((phenoxathiine-3- carbonyl)glycyl) pyrrolidine-2-carboxamide |
| 350 | 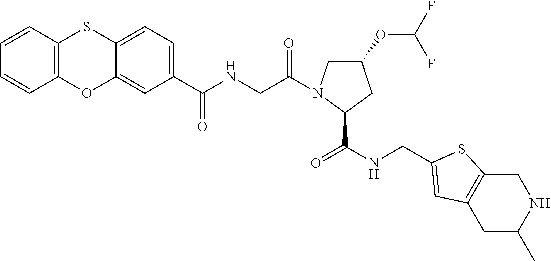 [SMILES: CC1Cc2cc(CNC(=O)[C@@H]3C[C@@H](OC(F)F)CN3C(=O)CNC(=O)c3ccc4c(c3)Oc3ccccc3S4)sc2CN1] | (2S,4R)-4-(difluoromethoxy)-N- ((5-methyl-4,5,6,7- tetrahydrothieno[2,3-c]pyridin-2- yl)methyl)-1-((phenoxathiine-3- carbonyl)glycyl) pyrrolidine-2-carboxamide |
| 351 | 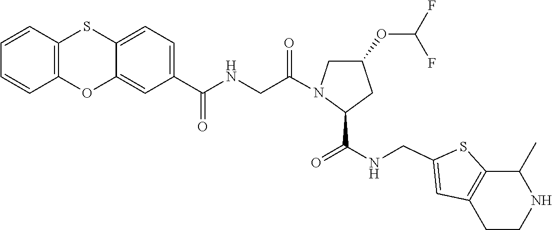 [SMILES: CC1NCCc2cc(CNC(=O)[C@@H]3C[C@@H](OC(F)F)CN3C(=O)CNC(=O)c3ccc4c(c3)Oc3ccccc3S4)sc21] | (2S,4R)-4-(difluoromethoxy)-N- ((7-methyl-4,5,6,7- tetrahydrothieno[2,3-c]pyridin-2- yl)methyl)-1-((phenoxathiine-3- carbonyl)glycyl) pyrrolidine-2-carboxamide |
| 352 | 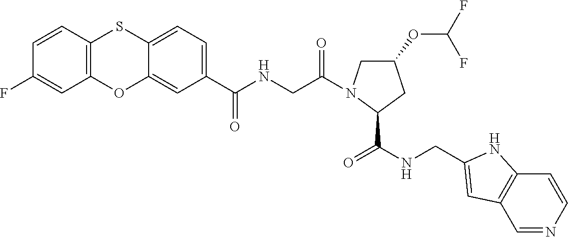 [SMILES: O=C(NCC(=O)N1C[C@H](OC(F)F)C[C@H]1C(=O)NCc1cc2cnccc2[nH]1)c1ccc2c(c1)Oc1cc(F)ccc1S2] | (2S,4R)-N-((1H-pyrrolo[3,2- c]pyridin-2-yl)methyl)-4- (difluoromethoxy)-1-((7- fluorophenoxathiine-3- carbonyl)glycyl)pyrrolidine-2- carboxamide |
| 353 | 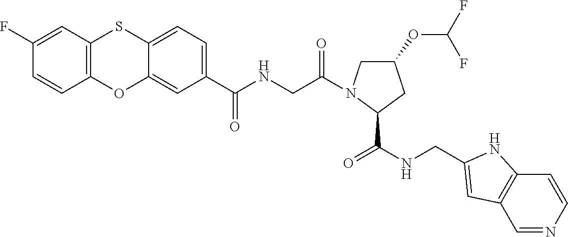 [SMILES: O=C(NCC(=O)N1C[C@H](OC(F)F)C[C@H]1C(=O)NCc1cc2cnccc2[nH]1)c1ccc2c(c1)Oc1ccc(F)cc1S2] | (2S,4R)-N-((1H-pyrrolo[3,2- c]pyridin-2-yl)methyl)-4- (difluoromethoxy)-1-((8- fluorophenoxathiine-3- carbonyl)glycyl) pyrrolidine-2-carboxamide |
| 354 | 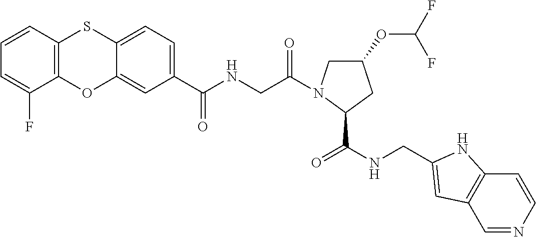 [SMILES: O=C(NCC(=O)N1C[C@H](OC(F)F)C[C@H]1C(=O)NCc1cc2cnccc2[nH]1)c1ccc2c(c1)Oc1c(F)cccc1S2] | (2S,4R)-N-((1H-pyrrolo[3,2- c]pyridin-2-yl)methyl)-4- (difluoromethoxy)-1-((6- fluorophenoxathiine-3- carbonyl)glycyl) pyrrolidine-2-carboxamide |
| 355 | 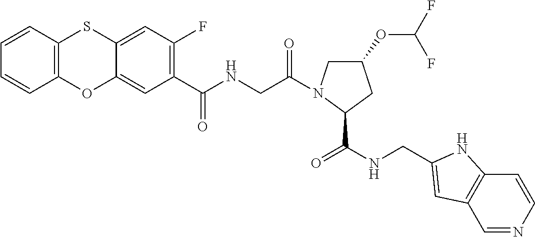 [SMILES: O=C(NCC(=O)N1C[C@H](OC(F)F)C[C@H]1C(=O)NCc1cc2cnccc2[nH]1)c1cc2c(cc1F)Sc1ccccc1O2] | (2S,4R)-N-((1H-pyrrolo[3,2- c]pyridin-2-yl)methyl)-4- (difluoromethoxy)-1-((2- fluorophenoxathiine-3- carbonyl)glycyl)pyrrolidine-2- carboxamide |
| 356 | 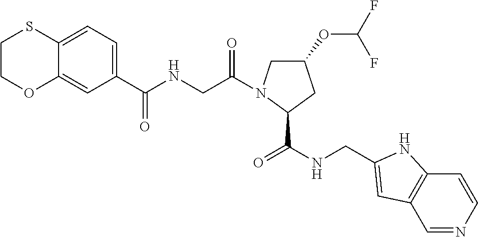 [SMILES: O=C(NCC(=O)N1C[C@H](OC(F)F)C[C@H]1C(=O)NCc1cc2cnccc2[nH]1)c1ccc2c(c1)OCCS2] | (2S,4R)-N-((1H-pyrrolo[3,2- c]pyridin-2-yl)methyl)-4- (difluoromethoxy)-1-((2,3- dihydrobenzo[b][1,4]oxathiine-7- carbonyl)glycyl) pyrrolidine-2-carboxamide |
| 357 | 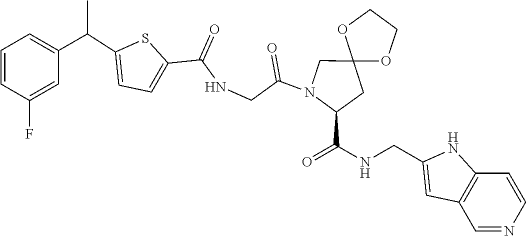 [SMILES: CC(c1cccc(F)c1)c1ccc(C(=O)NCC(=O)N2CC3(C[C@H]2C(=O)NCc2cc4cnccc4[nH]2)OCCO3)s1] | (8S)-N-((1H-pyrrolo[3,2- c]pyridin-2-yl)methyl)-7-((5-(1- (3-fluorophenyl)ethyl)thiophene- 2-carbonyl)glycyl)-1,4-dioxa-7- azaspiro[4.4]nonane-8- carboxamide |
| 358 | 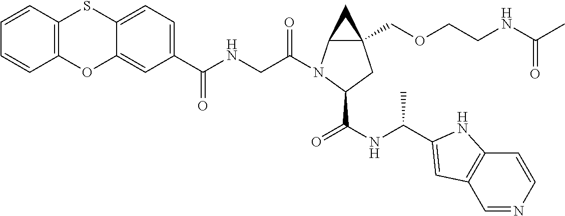 [SMILES: CC(=O)NCCOC[C@@]12C[C@@H]1N(C(=O)CNC(=O)c1ccc3c(c1)Oc1ccccc1S3)[C@H](C(=O)N[C@H](C)c1cc3cnccc3[nH]1)C2] | (1S,3S,5R)-N-((R)-1-(1H- pyrrolo[3,2-c]pyridin-2-yl)ethyl)- 5-((2-acetamidoethoxy)methyl)-2- ((phenoxathiine-3- carbonyl)glycyl)-2- azabicyclo[3.1.0]hexane-3- carboxamide |
| 359 | 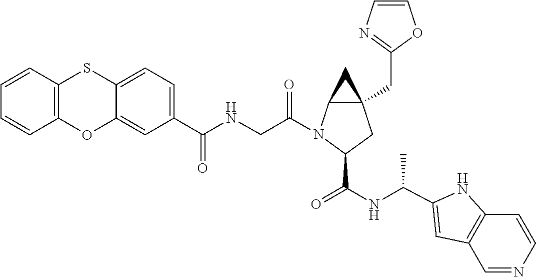 [SMILES: C[C@@H](NC(=O)[C@@H]1C[C@]2(Cc3ncco3)C[C@@H]2N1C(=O)CNC(=O)c1ccc2c(c1)Oc1ccccc1S2)c1cc2cnccc2[nH]1] | (1S,3S,5S)-N-((R)-1-(1H- pyrrolo[3,2-c]pyridin-2-yl)ethyl)- 5-(oxazol-2-ylmethyl)-2- ((phenoxathiine-3- carbonyl)glycyl)-2- azabicyclo[3.1.0]hexane-3- carboxamide |
| 360 | 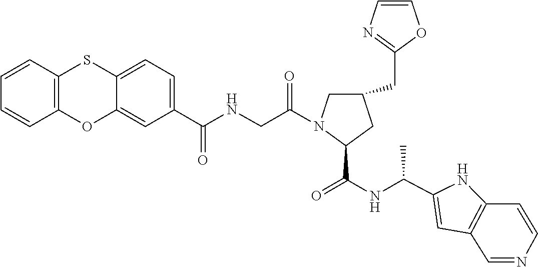 [SMILES: C[C@@H](NC(=O)[C@@H]1C[C@@H](Cc2ncco2)CN1C(=O)CNC(=O)c1ccc2c(c1)Oc1ccccc1S2)c1cc2cnccc2[nH]1] | (2S,4S)-N-((R)-1-(1H- pyrrolo[3,2-c]pyridin-2-yl)ethyl)- 4-(oxazol-2-ylmethyl)-1- ((phenoxathiine-3- carbonyl)glycyl)pyrrolidine-2- carboxamide |
| 361 | 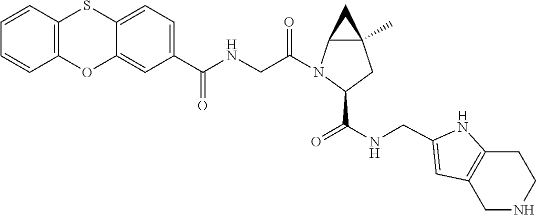 [SMILES: C[C@@]12C[C@@H]1N(C(=O)CNC(=O)c1ccc3c(c1)Oc1ccccc1S3)[C@H](C(=O)NCc1cc3c([nH]1)CCNC3)C2] | (1S,3S,5S)-5-methyl-2- ((phenoxathiine-3- carbonyl)glycyl)-N-((4,5,6,7- tetrahydro-1H-pyrrolo[3,2- c]pyridin-2-yl)methyl)-2- azabicyclo[3.1.0]hexane-3- carboxamide |
| 362 | 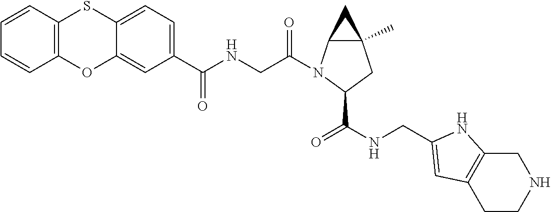 [SMILES: C[C@@]12C[C@@H]1N(C(=O)CNC(=O)c1ccc3c(c1)Oc1ccccc1S3)[C@H](C(=O)NCc1cc3c([nH]1)CNCC3)C2] | (1S,3S,5S)-5-methyl-2- ((phenoxathiine-3- carbonyl)glycyl)-N-((4,5,6,7- tetrahydro-1H-pyrrolo[2,3- c]pyridin-2-yl)methyl)-2- azabicyclo[3.1.0]hexane-3- carboxamide |
| 363 | 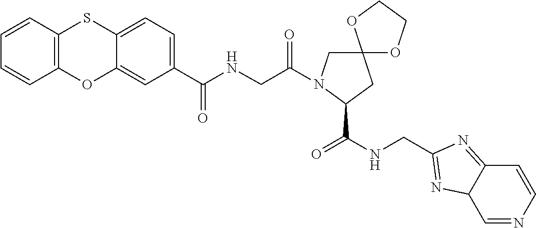 [SMILES: O=C(NCC(=O)N1CC2(C[C@H]1C(=O)NCC1=NC3C=NC=CC3=N1)OCCO2)c1ccc2c(c1)Oc1ccccc1S2] | (S)-N-(imidazo[1,2-c]pyrimidin- 2-ylmethyl)-7-((phenoxathiine-3- carbonyl)glycyl)-1,4-dioxa-7- azaspiro[4.4]nonane-8- carboxamide |
| 364 | 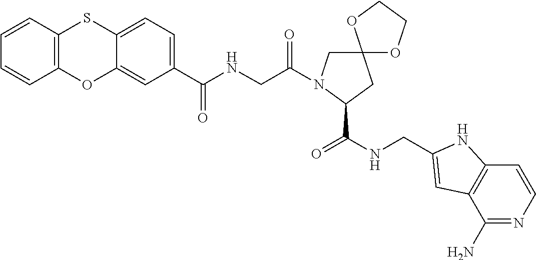 [SMILES: Nc1nccc2[nH]c(CNC(=O)[C@@H]3CC4(CN3C(=O)CNC(=O)c3ccc5c(c3)Oc3ccccc3S5)OCCO4)cc12] | (S)-N-((4-amino-1H-pyrrolo[3,2- c]pyridin-2-yl)methyl)-7- ((phenoxathiine-3- carbonyl)glycyl)-1,4-dioxa-7- azaspiro[4.4]nonane-8- carboxamide |
| 365 | 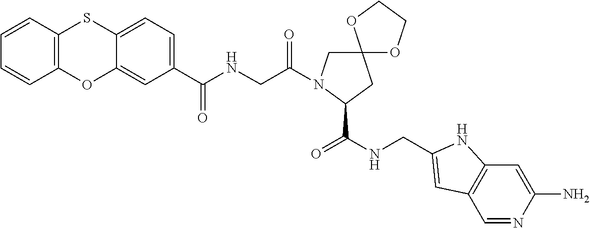 [SMILES: Nc1cc2[nH]c(CNC(=O)[C@@H]3CC4(CN3C(=O)CNC(=O)c3ccc5c(c3)Oc3ccccc3S5)OCCO4)cc2cn1] | (S)-N-((6-amino-1H-pyrrolo[3,2- c]pyridin-2-yl)methyl)-7- ((phenoxathiine-3- carbonyl)glcyl)-1,4-dioxa-7- azaspiro[4.4]nonane-8- carboxamide |
| 366 | 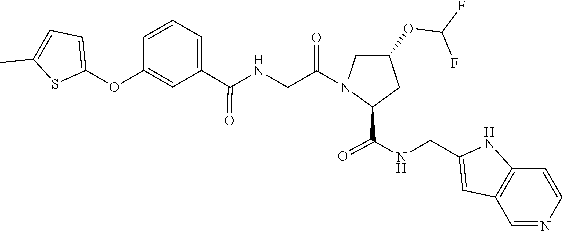 [SMILES: Cc1ccc(Oc2cccc(C(=O)NCC(=O)N3C[C@H](OC(F)F)C[C@H]3C(=O)NCc3cc4cnccc4[nH]3)c2)s1] | (2S,4R)-N-((1H-pyrrolo[3,2- c]pyridin-2-yl)methyl)-4- (difluoromethoxy)-1-((3-((5- methylthiophen-2- yl)oxy)benzoyl)glycyl)pyrrolidine- 2-carboxamide |
| 367 | 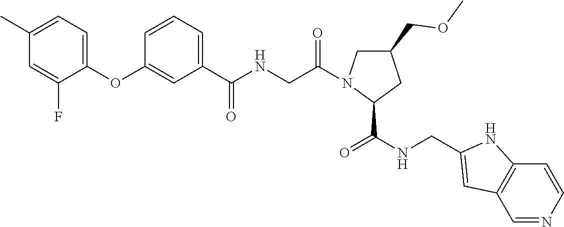 [SMILES: COC[C@H]1C[C@@H](C(=O)NCc2cc3cnccc3[nH]2)N(C(=O)CNC(=O)c2cccc(Oc3ccc(C)cc3F)c2)C1] | (2S,4S)-N-((1H-pyrrolo[3,2- c]pyridin-2-yl)methyl)-1-((3-(2- fluoro-4- methylphenoxy)benzoyl)glycyl)- 4-(methoxymethyl)pyrrolidine-2- carboxamide |
| 368 | 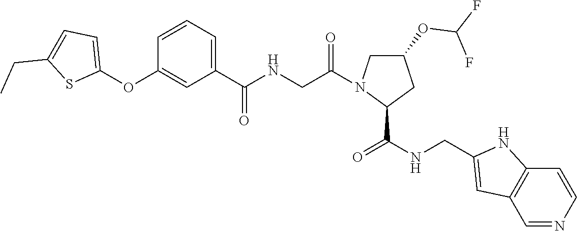 [SMILES: CCc1ccc(Oc2cccc(C(=O)NCC(=O)N3C[C@H](OC(F)F)C[C@H]3C(=O)NCc3cc4cnccc4[nH]3)c2)s1] | (2S,4R)-N-((1H-pyrrolo[3,2- c]pyridin-2-yl)methyl)-4- (difluoromethoxy)-1-((3-((5- ethylthiophen-2- yl)oxy)benzoyl)glycyl)pyrrolidine- 2-carboxamide |
| 369 | 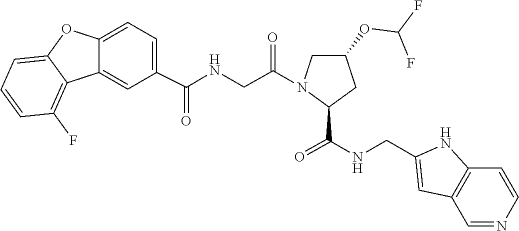 [SMILES: O=C(NCC(=O)N1C[C@H](OC(F)F)C[C@H]1C(=O)NCc1cc2cnccc2[nH]1)c1ccc2oc3cccc(F)c3c2c1] | (2S,4R)-N-((1H-pyrrolo[3,2- c]pyridin-2-yl)methyl)-4- (difluoromethoxy)-1-((9- fluorodibenzo[b,d]furan-2- carbonyl)glycyl)pyrrolidine-2- carboxamide |
| 370 | 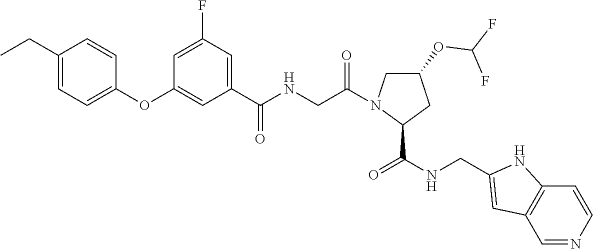 [SMILES: CCc1ccc(Oc2cc(F)cc(C(=O)NCC(=O)N3C[C@H](OC(F)F)C[C@H]3C(=O)NCc3cc4cnccc4[nH]3)c2)cc1] | (2S,4R)-N-((1H-pyrrolo[3,2- c]pyridin-2-yl)methyl)-4- (difluoromethoxy)-1-((3-(4- ethylphenoxy)-5- fluorobenzoyl)glycyl)pyrrolidine- 2-carboxamide |
| 371 | 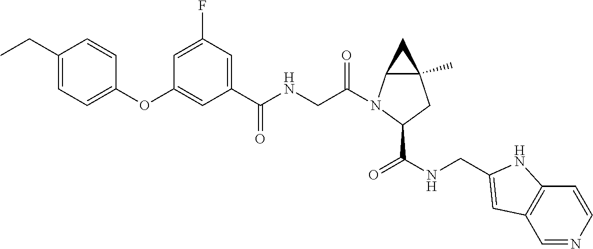 [SMILES: CCc1ccc(Oc2cc(F)cc(C(=O)NCC(=O)N3[C@H]4C[C@@]4(C)C[C@H]3C(=O)NCc3cc4cnccc4[nH]3)c2)cc1] | (1S,3S,5S)-N-((1H-pyrrolo[3,2- c]pyridin-2-yl)methyl)-2-((3-(4- ethylphenoxy)-5- fluorobenzoyl)glycyl)-5-methyl- 2-azabicyclo[3.1.0]hexane-3- carboxamide |
| 372 | 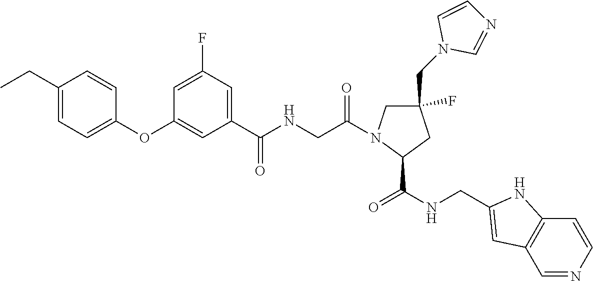 [SMILES: CCc1ccc(Oc2cc(F)cc(C(=O)NCC(=O)N3C[C@@](F)(Cn4ccnc4)C[C@H]3C(=O)NCc3cc4cnccc4[nH]3)c2)cc1] | (2S,4S)-4-((1H-imidazol-1- yl)methyl)-N-((1H-pyrrolo[3,2- c]pyridin-2-yl)methyl)-1-((3-(4- ethylphenoxy)-5- fluorobenzoyl)glycyl)-4- fluoropyrrolidine-2-carboxamide |
| 373 | 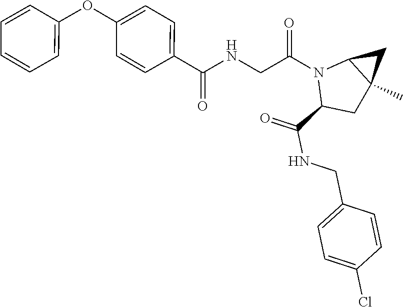 [SMILES: C[C@@]12C[C@@H]1N(C(=O)CNC(=O)c1ccc(Oc3ccccc3)cc1)[C@H](C(=O)NCc1ccc(Cl)cc1)C2] | (1S,3S,5S)-N-(4-chlorobenzyl)-5- methyl-2-((4- phenoxybenzoyl)glycyl)-2- azabicyclo[3.1.0]hexane-3- carboxamide |
| 374 | 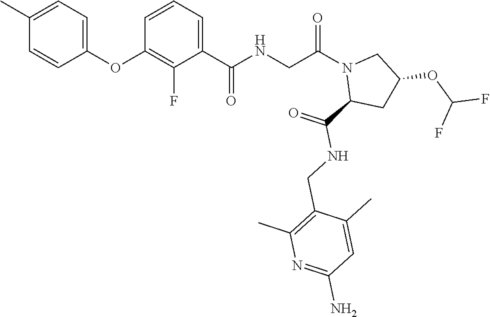 [SMILES: Cc1ccc(Oc2cccc(C(=O)NCC(=O)N3C[C@H](OC(F)F)C[C@H]3C(=O)NCc3c(C)cc(N)nc3C)c2F)cc1] | (2S,4R)-N-((6-amino-2,4- dimethylpyridin-3-yl)methyl)-4- (difluoromethoxy)-1-((2-fluoro-3- (p-tolyloxy)benzoyl)glycyl) pyrrolidine-2-carboxamide |
| 375 | 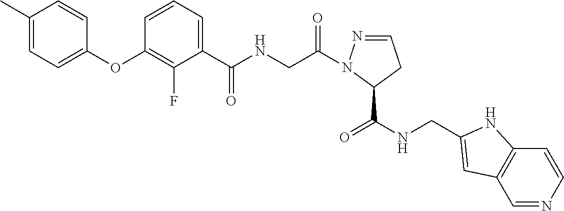 [SMILES: Cc1ccc(Oc2cccc(C(=O)NCC(=O)N3N=CC[C@H]3C(=O)NCc3cc4cnccc4[nH]3)c2F)cc1] | (S)-N-((1H-pyrrolo[3,2-c]pyridin- 2-yl)methyl)-1-((2-fluoro-3-(p- tolyloxy)benzoyl)glycyl)-4,5- dihydro-1H-pyrazole-5- carboxamide |
| 376 | 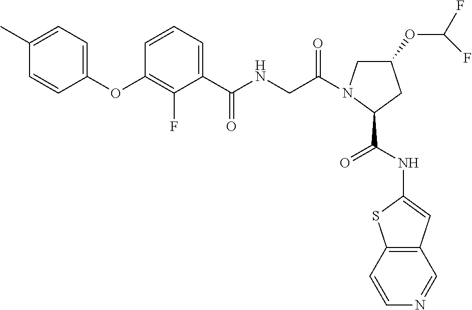 [SMILES: Cc1ccc(Oc2cccc(C(=O)NCC(=O)N3C[C@H](OC(F)F)C[C@H]3C(=O)Nc3cc4cnccc4s3)c2F)cc1] | (2S,4R)-4-(difluoromethoxy)-1- ((2-fluoro-3-(p- tolyloxy)benzoyl)glycyl)-N- (thieno[3,2-c]pyridin-2- yl)pyrrolidine-2-carboxamide |
| 377 | 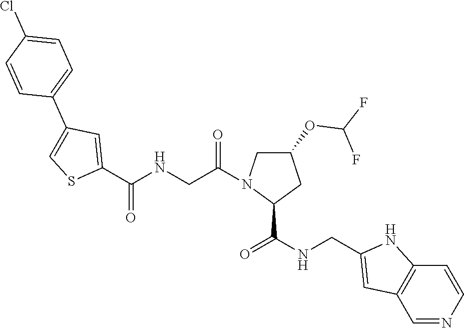 [SMILES: O=C(NCC(=O)N1C[C@H](OC(F)F)C[C@H]1C(=O)NCc1cc2cnccc2[nH]1)c1cc(-c2ccc(Cl)cc2)cs1] | (2S,4R)-N-((1H-pyrrolo[3,2- c]pyridin-2-yl)methyl)-1-((4-(4- chlorophenyl)thiophene-2- carbonyl)glycyl)-4- (difluoromethoxy)pyrrolidine-2- carboxamide |
| 378 | 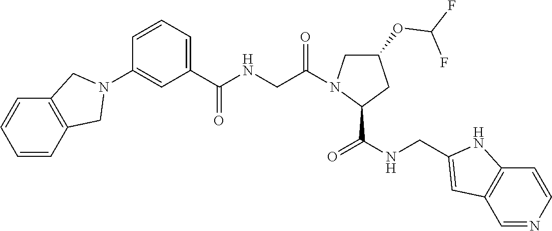 [SMILES: O=C(NCC(=O)N1C[C@H](OC(F)F)C[C@H]1C(=O)NCc1cc2cnccc2[nH]1)c1cccc(N2Cc3ccccc3C2)c1] | (2S,4R)-N-((1H-pyrrolo[3,2- c]pyridin-2-yl)methyl)-4- (difluoromethoxy)-1-((3- (isoindolin-2- yl)benzoyl)glycyl)pyrrolidine-2- carboxamide |
| 379 | 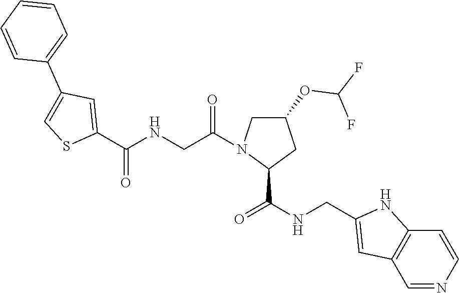 [SMILES: O=C(NCC(=O)N1C[C@H](OC(F)F)C[C@H]1C(=O)NCc1cc2cnccc2[nH]1)c1cc(-c2ccccc2)cs1] | (2S,4R)-N-((1H-pyrrolo[3,2- c]pyridin-2-yl)methyl)-4- (difluoromethoxy)-1-((4- phenylthiophene-2- carbonyl)glycyl)pyrrolidine-2- carboxamide |
| 380 | 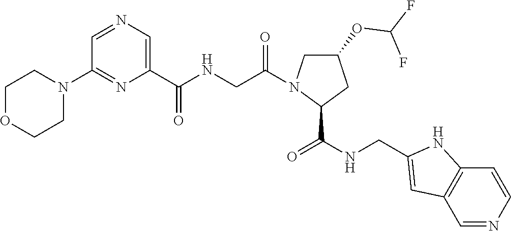 [SMILES: O=C(NCC(=O)N1C[C@H](OC(F)F)C[C@H]1C(=O)NCc1cc2cnccc2[nH]1)c1cncc(N2CCOCC2)n1] | N-(2-((2S,4R)-2-(((1H- pyrrolo[3,2-c]pyridin-2- yl)methyl)carbamoyl)-4- (difluoromethoxy)pyrrolidin-1- yl)-2-oxoethyl)-6- morpholinopyrazine-2- carboxamide |
| 381 | 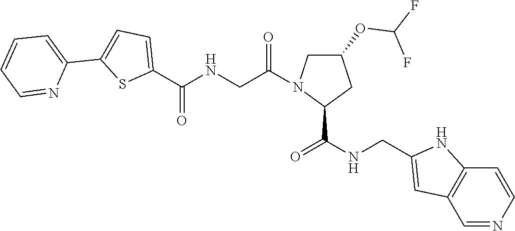 [SMILES: O=C(NCC(=O)N1C[C@H](OC(F)F)C[C@H]1C(=O)NCc1cc2cnccc2[nH]1)c1ccc(-c2ccccn2)s1] | (2S,4R)-4-(difluoromethoxy)-1- [2-[[5-(2-pyridyl)thiophene-2- carbonyl]amino]acetyl]-N-(1H- pyrrolo[3,2-c]pyridin-2- ylmethyl)pyrrolidine-2- carboxamide |
| 382 | 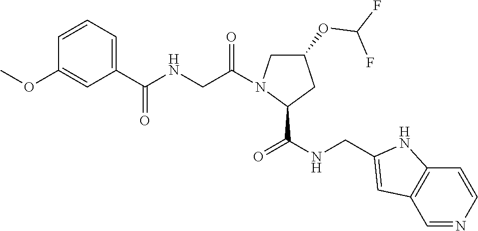 [SMILES: COc1cccc(C(=O)NCC(=O)N2C[C@H](OC(F)F)C[C@H]2C(=O)NCc2cc3cnccc3[nH]2)c1] | (2S,4R)-N-((1H-pyrrolo[3,2- c]pyridin-2-yl)methyl)-4- (difluoromethoxy)-1-((3- methoxybenzoyl)glycyl) pyrrolidine-2-carboxamide |
| 383 | 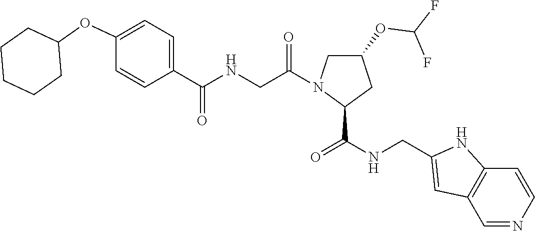 [SMILES: O=C(NCC(=O)N1C[C@H](OC(F)F)C[C@H]1C(=O)NCc1cc2cnccc2[nH]1)c1ccc(OC2CCCCC2)cc1] | (2S,4R)-N-((1H-pyrrolo[3,2- c]pyridin-2-yl)methyl)-1-((4- (cyclohexyloxy)benzoyl)glycyl)- 4-(difluoromethoxy)pyrrolidine-2- carboxamide |
| 384 | 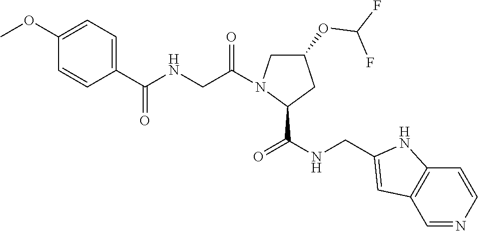 [SMILES: COc1ccc(C(=O)NCC(=O)N2C[C@H](OC(F)F)C[C@H]2C(=O)NCc2cc3cnccc3[nH]2)cc1] | (2S,4R)-N-((1H-pyrrolo[3,2- c]pyridin-2-yl)methyl)-4- (difluoromethoxy)-1-((4- methoxybenzoyl)glycyl) pyrrolidine-2-carboxamide |
| 385 | 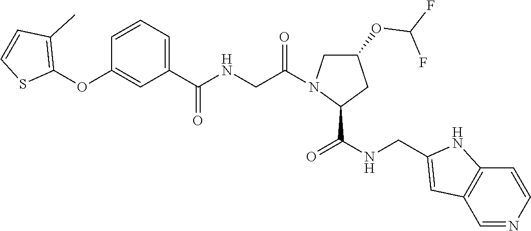 [SMILES: Cc1ccsc1Oc1cccc(C(=O)NCC(=O)N2C[C@H](OC(F)F)C[C@H]2C(=O)NCc2cc3cnccc3[nH]2)c1] | (2S,4R)-N-((1H-pyrrolo[3,2- c]pyridin-2-yl)methyl)-4- (difluoromethoxy)-1-((3-((3- methylthiophen-2- yl)oxy)benzoyl)glycyl) pyrrolidine-2-carboxamide |

### Example 3. Human C1s Enzyme Assay

The IC₅₀ values of the compounds of Table 1 were determined with the human C1s enzyme assay described below. Other standard complement assays are also available.

Human complement C1s enzyme (purified from human serum, Complement Technology, Inc.) at 1.16 nM final concentration was incubated with test compound at various concentrations for 5 min at room temperature in 50 mM Tris, 1 M NaCl, pH 7.5. A synthetic substrate Z-L-Lys-SBzl and DTNB (Ellman's reagent) were added to final concentrations of 100 μM each. Absorbance at 405 nm (A405) was recorded at 30 second intervals for 30 min using a microplate spectrophotometer. IC₅₀ values were calculated by nonlinear regression of complement C₁ s reaction rates as a function of test compound concentration.

Table 2 shows the IC₅₀ values of the compounds obtained from the above-described human CIs enzyme assay. Three ***s are used to denote compounds with an IC₅₀ less than 100 nanomolar; two **s indicates a compound with an IC₅₀ greater than 100 nanomolar and less than 1 micromolar, one denotes compounds with an IC₅₀ greater than 1 micromolar. ND indicates a compound for which the IC₅₀ value is not determined.

TABLE 2
C1s Inhibiting Activity of Compounds of the Present Disclosure

| Cmpd # | IC₅₀ (Stars) | Cmpd # | IC₅₀ (Stars) | Cmpd # | IC₅₀ (Stars) | Cmpd # | IC₅₀ (Stars) | Cmpd # | IC₅₀ (Stars) |
|---|---|---|---|---|---|---|---|---|---|
| 1 | * | 2 | *** | 3 | * | 4 | *** | 5 | *** |
| 6 | *** | 7 | *** | 8 | *** | 9 | ** | 10 | * |
| 11 | * | 12 | * | 13 | * | 14 | * | 15 | * |
| 16 | * | 17 | *** | 18 | *** | 19 | * | 20 | *** |
| 21 | * | 22 | ** | 23 | * | 24 | *** | 25 | *** |
| 26 | *** | 27 | *** | 28 | *** | 29 | * | 30 | *** |
| 31 | * | 32 | *** | 33 | *** | 34 | * | 35 | *** |
| 36 | *** | 37 | *** | 38 | *** | 39 | *** | 40 | ** |
| 41 | * | 42 | * | 43 | * | 44 | *** | 45 | * |
| 46 | * | 47 | *** | 48 | * | 49 | * | 50 | ** |
| 51 | * | 52 | * | 53 | * | 54 | *** | 55 | * |
| 56 | ** | 57 | * | 58 | *** | 59 | * | 60 | * |
| 61 | * | 62 | * | 63 | * | 64 | * | 65 | * |
| 66 | * | 67 | ** | 68 | ** | 69 | * | 70 | * |
| 71 | *** | 72 | *** | 73 | *** | 74 | ** | 75 | *** |
| 76 | * | 77 | *** | 78 | ** | 79 | ** | 80 | *** |
| 81 | *** | 82 | ** | 83 | ** | 84 | * | 85 | * |
| 86 | ** | 87 | ** | 88 | *** | 89 | * | 90 | *** |
| 91 | *** | 92 | ** | 93 | *** | 94 | * | 95 | * |
| 96 | * | 97 | * | 98 | ** | 99 | *** | 100 | ** |
| 101 | *** | 102 | *** | 103 | ** | 104 | ** | 105 | *** |
| 106 | *** | 107 | *** | 108 | ** | 109 | * | 110 | * |
| 111 | *** | 112 | *** | 113 | *** | 114 | ** | 115 | * |
| 116 | * | 117 | ** | 118 | * | 119 | * | 120 | *** |
| 121 | ** | 122 | ** | 123 | ** | 124 | *** | 125 | *** |
| 126 | *** | 127 | *** | 128 | * | 129 | *** | 130 | *** |
| 131 | *** | 132 | *** | 133 | *** | 134 | * | 135 | * |
| 136 | ** | 137 | *** | 138 | * | 139 | ** | 140 | ** |
| 141 | *** | 142 | *** | 143 | *** | 144 | *** | 145 | *** |
| 146 | *** | 147 | ** | 148 | *** | 149 | *** | 150 | *** |
| 151 | ** | 152 | *** | 153 | *** | 154 | *** | 155 | ** |
| 156 | ** | 157 | *** | 158 | *** | 159 | * | 160 | *** |
| 161 | *** | 162 | ** | 163 | * | 164 | *** | 165 | *** |
| 166 | *** | 167 | *** | 168 | *** | 169 | *** | 170 | *** |
| 171 | *** | 172 | *** | 173 | *** | 174 | *** | 175 | *** |
| 176 | *** | 177 | *** | 178 | *** | 179 | *** | 180 | *** |
| 181 | *** | 182 | *** | 183 | *** | 184 | *** | 185 | *** |
| 186 | *** | 187 | *** | 188 | *** | 189 | * | 190 | *** |
| 191 | *** | 192 | * | 193 | * | 194 | * | 195 | * |
| 196 | * | 197 | * | 198 | * | 199 | * | 200 | *** |
| 201 | *** | 202 | *** | 203 | *** | 204 | *** | 205 | ** |
| 206 | *** | 207 | * | 208 | *** | 209 | ** | 210 | *** |
| 211 | *** | 212 | *** | 213 | *** | 214 | * | 215 | *** |
| 216 | *** | 217 | *** | 218 | *** | 219 | *** | 220 | *** |
| 221 | * | 222 | * | 223 | ** | 224 | * | 225 | * |
| 226 | * | 227 | * | 228 | *** | 229 | * | 230 | * |
| 231 | *** | 232 | * | 233 | * | 234 | *** | 235 | *** |
| 236 | * | 237 | * | 238 | *** | 239 | * | 240 | *** |
| 241 | *** | 242 | * | 243 | * | 244 | *** | 245 | * |
| 246 | *** | 247 | ** | 248 | * | 249 | * | 250 | * |
| 251 | *** | 252 | *** | 253 | ** | 254 | *** | 255 | ** |
| 256 | *** | 257 | * | 258 | * | 259 | *** | 260 | *** |
| 261 | *** | 262 | * | 263 | * | 264 | * | 265 | * |
| 266 | *** | 267 | * | 268 | * | 269 | *** | 270 | *** |
| 271 | *** | 272 | *** | 273 | *** | 274 | *** | 275 | *** |
| 276 | * | 277 | *** | 278 | *** | 279 | * | 280 | ** |
| 281 | ** | 282 | * | 283 | *** | 284 | *** | 285 | * |
| 286 | *** | 287 | *** | 288 | *** | 289 | *** | 290 | * |
| 291 | * | 292 | *** | 293 | *** | 294 | *** | 295 | *** |
| 296 | * | 297 | *** | 298 | *** | 299 | *** | 300 | *** |
| 301 | *** | 302 | *** | 303 | *** | 304 | *** | 305 | *** |
| 306 | *** | 307 | *** | 308 | *** | 309 | *** | 310 | *** |
| 311 | *** | 312 | *** | 313 | *** | 314 | *** | 315 | *** |
| 316 | *** | 317 | *** | 318 | *** | 319 | *** | 320 | *** |
| 321 | *** | 322 | *** | 323 | *** | 324 | *** | 325 | *** |
| 326 | ND | 327 | *** | 328 | *** | 329 | *** | 330 | ND |
| 331 | ND | 332 | *** | 333 | ND | 334 | *** | 335 | ND |
| 336 | *** | 337 | ND | 338 | *** | 339 | *** | 340 | ND |
| 341 | ND | 342 | ND | 343 | ND | 344 | ND | 345 | ND |
| 346 | * | 347 | * | 348 | ND | 349 | ND | 350 | ND |
| 351 | ND | 352 | ND | 353 | ND | 354 | ND | 355 | *** |
| 356 | *** | 357 | ** | 358 | ND | 359 | ND | 360 | ND |
| 361 | ND | 362 | ND | 363 | ND | 364 | ND | 365 | ND |
| 366 | *** | 367 | *** | 368 | *** | 369 | *** | 370 | *** |
| 371 | *** | 372 | *** | 373 | * | 374 | * | 375 | *** |
| 376 | * | 377 | *** | 378 | *** | 379 | *** | 380 | * |
| 381 | *** | 382 | ** | 383 | *** | 384 | * | 385 | *** |

### Example 4. Hemolysis Assay

The IC₅₀ values of the compounds of Tabel 1 were also determined using a hemolysis assay. The hemolysis assay was previously described by Dodds, A. W. and Sim, R. B. (1997); Morgan, B. P. (2000). Prior to the assay, the optimum concentration of Normal Human Serum (NHS) needed to achieve 100% lysis of antibody sensitized sheep erythrocytes (EA) is determined by titration. EA are sheep erythrocytes with rabbit IgM anti-sheep erythrocyte antibodies bound to their surface. In the assay, NHS (Complement Technology) is diluted in GVB++ Buffer (0.1% gelatin, 5 mM Veronal, 145 mM NaCl, 0.025% NaN₃, pH 7.3, 0.15 mM calcium chloride and 0.5 mM magnesium chloride, Complement Technology) and incubated with test compound at various concentrations for 2 min at room temperature. EA (Complement Technology) freshly suspended in GVB++ are added to a final concentration of 1×10⁸ cells/mL and reactions are incubated for 60 min at 37° C. Positive control reactions (100% lysis) consist of GVB++ with NHS and EA but without test compound; negative control reactions (0% lysis) consist of GVB++ with EA only. Samples are centrifuged at 2000 g for 3 min and supernatants collected. Absorbance at 405 nm (A405) is recorded using a microplate spectrophotometer. IC₅₀ values are calculated by nonlinear regression from the percentage of hemolysis as a function of test compound concentration.

This specification has been described with reference to various specific embodiments. However, one of ordinary skill in the art appreciates that various modifications and changes can be made without departing from the scope of the invention as set forth inthe claims. Accordingly, the specification is to be regarded in an illustrative rather than a restrictive sense, and all such modifications are intended to be included within the scope of the claims.

### Example 5. Microsomal Stability Assay

The microsomal stability of compounds of the disclosure are analyzed as described below.

### Materials

| Chemicals and Reagents: | Product Number |
|---|---|
| Deionized Water [H₂O] | |
| Acetonitrile [ACN] (HPLC Grade) | Sigma Aldrich #34998 |
| Dimethyl Sulfoxide [DMSO] | Sigma Aldrich #D8418 |
| Wistar-Han Rat Liver Microsomes [RLM] | XenoTech #R6000 |
| Beagle Dog Liver Microsomes [DLM] | XenoTech #D1000 |
| Cynomolgus Monkey Liver Microsomes | XenoTech #P2000 |
| [MLM] | |
| Human Liver Microsomes [HLM] | XenoTech #H2610 |
| β-Nicotinamide adenine dinucleotide | Sigma Aldrich #N3886 |
| phosphate sodium salt [NADP+] | |
| Glucose-6-phosphate | Sigma Aldrich #G7879 |
| Glucose-6-phosphate dehydrogenase | Sigma Aldrich #G6378 |
| Magnesium Chloride [MgCl₂] | Sigma Aldrich #M8266 |
| Phosphate Buffer [0.5 mM] | Corning Life Sciences |
| | #451201 |

### Methodology

The Phosphate Buffer solution is prepared by mixing 15.2 mL of deionized water with 1.9 mL of 0.5 M Phosphate Buffer and 1.9 mL of 33 mM MgCl₂. The Microsomal Working solution is prepared by mixing 14.655 mL of deionized water with 1.9 mL of 0.5 mM Phosphate Buffer, 1.9 mL of 33 mM MgCl₂, and 545 μL of 20 mg/mL of liver microsomes (species specific). The Cofactor Solution is prepared by mixing 8.12 mL of deionized water with 1.45 mL of 0.5 mM Phosphate Buffer, 1.45 mL of 33 nM MgCl₂, 1.16 mL of 100 mM NADP+ solution, 1.16 mL of 100 mM Glucose-6-Phosphate solution, and 1.16 mL of 100 U/mL Glucose-6-Phosphate Dehydrogenase solution. Final assay reagent concentrations are 0.1 μM Test Article, 0.5 mg/mL liver microsomes, 1 mM NADP+, 5 mM Glucose-6-Phosphate, 1 U/mL Glucose-6-phosphate dehydrogenase, 50 mM Potassium Phosphate Buffer (pH 7.4), 3.3 mM MgCl₂, and 0.2% DMSO.

The assay reaction mixture is prepared by taking 436 μL of Microsomal Working Solution and mixing it with 63 μL of Cofactor Solution in a 1.5 mL 96-well plate and preincubated at 37° C. for 5 minutes. The assay was initiated by the addition of 1 μL of 50 μM solution of a test compound in DMSO.

At 0 minutes, 30 minutes, and 120 minutes, 100 μL aliquots of the incubation mixture are removed and mixed with 200 μL of cold acetonitrile containing 0.2 μM Tolbutamide (internal standard) in a separate 96-well plate. At the conclusion of the assay, the sample collection plate are vortexed for thirty seconds, and centrifuged at 3000×g and 4° C. for 10 minutes. The supernatants were transferred to clean 96-well bioanalysis plates for analysis and stored at 4° C.

### Sample Bioanalysis

The test compound concentrations in liver microsome samples are analyzed by a LC-MS/MS method on a Sciex API 5500 Q-Trap mass spectrometer using turbo ion spray with MRM monitoring in the positive mode. Analyst© software (Version 1.6.2) was used to capture the LC-MS/MS data and integrate the peak areas.

### Example 6. Hepatic Clearance Assay

The hepatic clearance of compounds of the present disclosure are analyzed as described below.

### Thawing Media and Test Article Preparation

The thawing media is warmed in a water bath 37° C. The test compounds are diluted 200 × from 20 mM to 100 μM in DMSO, then to 0.2 M by adding 3 μL of test compound solution into 1.5 mL of Williams E medium. Midazolam (1 mM) and 7-ethoxycoumarin (100 mM) controls are thawed, and 3 μL of each control is added to 1.5 mL Williams E medium. Cold acetonitrile (150 μL) with internal standards are then added to a 96-well plate.

### Cell Viability Determination

Cryopreserved hepatocytes (human, rat, dog, or monkey) are thawed in water bath for˜90 s, transferred into warm thawing media, and centrifuged at 100 g for 5 min. The supernatant fluid is then aspirated, and the pellets are resuspended in 5 mL Williams E medium. The cell suspension (50 μL) is mixed with Trypan blue, then an aliquot is placed on a hemacytometer for cell viability determination. If the cell viability is determined to be over 70%, the cell concentration in the hepatocyte medium is then adjusted to 10⁶/mL by adding the appropriate amount of Williams E medium.

### Incubation

The hepatocyte suspension (500 μL) is added to each well of 24-well plate. The test compound solution (500 μL) is then added. At 0 min, 75 μL of the sample is taken and quenched with 150 μL cold acetonitrile in the 96-well plate. Samples are taken and quenched at 30 min, 50 min, and 120 min (15 min and 120 min for 7-ethoxycoumarin). After incubation and quenching, the samples are stored at −20° C. overnight.

### LC-MS/MS Analysis

After the mass spectrometer is tuned for each compound using a 2 mL solution of 0.05 MS tuning for each compound 0.05 μM of the test compound in 1:1 Water/Acetonitrile, the 96-well plate containing the incubation samples and quench solutions is centrifuged at 25° C., 3000 rpm for 15 min. The samples (160 μL) are transferred to deep well pates and queued in LC-MS/MS for analysis. To determine the Intrinsic clearance and hepatic clearance (% Qh) of the test compounds, the in peak area ratio (compound peak area/internal standard peak area) is plotted against time and the gradient of the line determined.

### Example 7. Caco-2 Permeability Assay

The bidirectional permeability and efflux probability of compounds of the present disclosure in Caco-2 cell monolayers with and without efflux inhibitors are determined following the procedure described below.

### Materials and Reagents

Caco-2 cells are obtained from the American type culture collection (ATCC), and the ATCC® Number is HTB-37. Hepes, Penicillin, Streptomycin. Trypsin/EDTA and DMSO are purchased from Solarbio. Bovine Serum Albumin (BSA) is purchased from Solarbio Biotechnology Co., Ltd. Fetal bovine serum, Hank's balanced salt solution (HBSS) and Non-essential amino acids (NEAA) are all purchased from Gibco by Thermo Fisher Scientific. Dulbecco's Modified Eagle's Medium (DMEM) is purchased from Corning Corporation or Hyclone. HTS Transwell-96 Well (Cat. No. 3391) Permeable Supports and other sterile plastic ware are purchased from Corning Corporation. Millicell Epithelial Volt-Ohm measuring system is purchased from Millipore. Cellometer® Vision is purchased from Nexcelom Bioscience LLC. Infinite 200 PRO microplate reader is purchased from Tecan. MTS2/4 orbital shaker is purchased from IKA Labortechnik.

Transport buffer: HESS containing 25 mM HEPES, pH 7.4 and 3% BSA: to prepare the HBSS containing 25 mM HEPES, pH 7.4, accurately weigh 5.958 g of HEPES and 0.35 g sodium hydrogen carbonate and add into 900 mL of pure water, then sonicate to dissolve the content. Transfer 100 mL of 10× HBSS into the solution, and place the solution on a stirrer, slowly adjust pH with sodium hydrate to 7.4, following with filtering and add 3% BSA.

Transport buffer with efflux inhibitors: HBSS containing 25 mM HEPES, pH 7.4 and 3% BSA: to prepare the HBSS containing 25 mM HEPES, pH 7.4, accurately weigh 5.958 g of HEPES and 0.35 g sodium hydrogen carbonate and add into 900 mL, of pure water, then sonicate to dissolve the content. Transfer 100 mL of 10× HBSS into the solution, and place the solution on a stirrer, slowly adjust pH with sodium hydrate to 7.4, following with filtering. Before the experiment, add the 10 mM zosuquidar, 30 mM benzbromarone and 2 mM KO-143 into the HBSS (25 mM HEPES, pH 7.4) with 1000-fold dilution and 3% BSA at a final concentration of 1 μM zosuquidar, 30 μM benzbromarone, and 2 μM KO-143.

### Preparation for Cell Seeding

1. Prepare transport buffer (HBSS containing 25 mM HEPES, pH 7.4): accurately weigh 5.958 g of HEPES and 0.35 g sodium hydrogen carbonate and add into 900 mL of pure water, then sonicate to dissolve the content. Transfer 100 mL of 10× HBSS into the solution, and place the solution on a stirrer, slowly adjust pH with sodium hydroxide to 7.4, following with filtering.

2. Prepare Caco-2 cell culture medium consisting of Dulbecco's Modified Eagle's Medium (DMEM) with high glucose and L-glutamine supplemented with 10% FBS, 0.1 mg/mL of streptomycin, 100 units of penicillin, 0.6 g/mL of Kanamycin sulfate and 1× non-essential amino acids (NEAA).

3. Add 50 μL of culture medium to each well of the Transwell insert. Remove Transwell insert from reservoir and add 25 mL of culture medium.

4. Incubate at 37° C., 5% CO₂ for 1 hour. Plates are ready for cell seeding.

5. Cultivate cells in T-75 flasks in a cell culture incubator set at 37° C., 5% CO₂, 95% relative humidity. Allow cells to reach 80-90% confluence before detaching and splitting.

6. Rinse cultivated cells in T-75 flasks with 5 mL, PBS. Aspirate off, add 1.5 mL trypsin/EDTA, and incubate at 37° C. for approximately 5 to 10 minutes or until the cells detach and float. Inactivate trypsin/EDTA by adding excess medium containing serum.

7. Transfer cell suspension to a conical tube and pellet cells by centrifugation at 120×g for 10 minutes.

8. Resuspend cells in seeding medium at a density of 6.86×10⁵ cells/mL. This cell concentration can be used to seed 2.4×10⁵ cells/cm².

Seeding and Feeding of Caco-2 Cells into Transwell Plates

1. Add 50 μL of above cell suspension to each well of a previously prepared Transwell plate.

2. Incubate the plate for 14-18 days. Replace the medium every other day, beginning no sooner than 48 hours after initial plating. Medium must be replaced on the day before conducting the experiment.

3. The procedure for medium changes is as follows. Remove the plate from incubator and place in hood. Aspirate the medium from reservoir and each Transwell insert. Add 100 μL of culture medium to each well of Transwell inserts and 25 mL of culture medium to reservoir tray. Return plate to incubator.

### Assessment of Cell Monolayer Integrity

1. When the 14-day cultured Caco-2 cells have reached confluence and are differentiated, they are ready to be used for transport studies.

2. Measure the electrical resistance across the monolayer using Millicell Epithelial Volt-Ohm measuring system (Millipore).

3. Record the electrical resistance for each well.

4. Once all wells have been measured, return plate to incubator.

5. Calculation of TEER values: TEER measurement (ohms)×Area of membrane (cm²)=TEER value (ohm·cm²). Any monolayer with a TEER value <230 ohms·cm², indicating poor monolayer formation, will be discarded.

### Performing the Drug Transport Assay

1. Remove the Caco-2 plate from the incubator. Next, wash the monolayer, exchange the volume two times using pre-warmed transport buffer. Then, incubate the plate at 37° C. for 30 minutes.

2. For test compounds and control compound digoxin, prepare stock solutions in DMSO at 2 mM and dilute by transport buffer with or without efflux Inhibitors to get 10 M working solution. For control compound metoprolol, prepare stock solutions in DMSO at 2 mM and dilute by HBSS without 3% BSA to get 10 M working solution. The final DMSO concentration is 0.8%.

3. Prepare bulk donor solutions with 597 μL transport buffer (with or without efflux inhibitors) and 3 μL 2 mM test compound or control compound.

4. Prepare bulk receiver solutions with 597 μL transport buffer (with or without efflux inhibitors) and 3 μL DMSO.

5. To determine the rate of drug transport in the apical to basolateral direction. Add 108 μL of the donor solution to the Transwell insert (apical compartment), and transfer 8 μL sample immediately from the apical compartment to 72 μL transport buffer and 240 μL quenching solvents in a new 96-well plate as the initial donor sample (A-B). Shake the plate at 1000 rpm for 5 minutes.

Fill the wells in the receiver plate (basolateral compartment) with 300 μL of receiver solution.

6. To determine the rate of drug transport in the basolateral to apical direction. Add 308 μL of the donor solution to the receiver plate wells (basolateral compartment), and transfer 8 μL sample immediately from the basolateral compartment to 72 μL transport buffer and 240 μL quenching solvents in a new 96-well plate as the initial donor sample (B-A). Fill the Transwell insert (apical compartment) with 100 μL of receiver solution. Shake the plate at 1000 rpm 5 minutes.

7. Incubate at 37° C. for 2 hours. At the end of the transport period, transfer 8 μL of samples from donor sides (apical compartment for Ap→B1 flux, and basolateral compartment for B1→Ap flux) to 72 μL transport buffer and 240 μL quenching solvents in a new 96-well plate. Remove 80 μL directly from receiver sides (basolateral compartment for Ap→B1 flux, and apical compartment for B1→Ap flux) and transfer to new 96-well plates with 240 μL quenching solvents. Vortex at 1000 rpm for 10 minutes. Centrifuge the quenching plates of each time point for 30 minutes at 4,000 rpm and 4° C. Transfer 100 μL of supernatant of each compound into a 96-well analysis plate containing 100 μL of pure water in the corresponding wells. Shake the analysis plate at 1,000 rpm for 2 minutes and the sample is analyzed by LC-MS/MS. All incubations are performed in duplicate.

8. To determine the Lucifer yellow leakage after 2-hour transport period, prepare stock solutions of Lucifer yellow in water and dilute with transport buffer to reach the final concentration of 100 μM. Add 100 μL of the Lucifer yellow solution to the Transwell insert (apical compartment). Fill the wells in the receiver plate (basolateral compartment) with 300 μL of transport buffer. Incubate at 37° C. for 30 minutes. Remove 80 μL directly from the apical and basolateral wells (using the basolateral access holes) and transfer to new 96 wells plates. Measure Lucifer Yellow fluorescence (to monitor monolayer integrity) in a fluorescence plate reader at 485 nm excitation and 530 nm emission.

### Example 8. Pharmacokinetic Profile following IV and PO Administration in Rats

The pharmacokinetic profile of compounds of the present disclosure following intravenous (IV) or oral (PO) administration in male Wistar Han rats at various dosages is assessed based on the general procedure described below.

### Exemplary Dosing Information

### IV Dosing:

For Male Wistar Han rats previously fitted with a jugular vein catheter (JVC) for blood collection, the animals will receive a single IV dose of a test compound (e.g., at a dose level of 0.5 mg/kg or more). Sampling will be done from the cannulated carotid artery.

### Po Dosing:

For Male Wistar Han rats previously fitted with a jugular vein catheter for blood collection animals will receive a single oral dose of compound, e.g., at a dose level of 5 mg/kg or more.

### Animal Feeding Control:

All animals for IV and PO administration will be fasted overnight and fed after 4 hours collection post dosing.

### Dose Formulation Processing During Dosing:

The dose formulations will be kept at room temperature and administered within 2 hours after preparation.

### Exemplary Pharmacokinetic (PK) Schedule:

### IV Dosing:

Plasma: Pre-dose, 0.33, 0.083, 0.25, 0.5, 1, 2, 4, 8, and 24 hours pose dose.

### Po Dosing:

Plasma: Pre-dose, 0.25, 0.5, 1, 2, 4, 8, and 24 hours post dose.

### PK Samples Analyses

Concentrations of test compounds in the plasma samples will be analyzed using a LC-MS/MS method. WinNonlin (Phoenix™, version 8.3) or other a similar software will be used for pharmacokinetic calculations. The following pharmacokinetic parameters will be calculated, whenever possible, from the plasma concentrations versus time data:
IV administration: T_{1/2}, C₀, AUCₗₐₛₜ, AUC_{inf}, MIRT, Cl, Vss
PO administration: T_{1/2}, Cₘₐₓ, Tₘₐₓ, AUC_{inf}, AUCₗₐₛₜ, F %

### Example 9. MASP-2 Protease Assay

Compounds of the present disclosure are assessed with a MASP-2 protease assay.

### Materials

| | |
|---|---|
| Buffer | 20 mM Hepes, 140 mM NaCl, pH 7.4 |
| Enzyme | MASP-2 (CCP2-SP) (Charles River) |
| Substrate | Z-Lys-SBzl (Sigma Aldrich) |
| Detection | DTNB Ellman's Reagent (Sigma Aldrich) |

### Method

The test compounds and/or DMSO are preincubated with enzyme in buffer for 5 minutes at Room temperature (RT), and also without enzyme as a control. The reaction is initiated by addition of substrate and DTNB mix. In this assay, the concentrations of the enzyme, substrate, and DTNB concentration are 16 nM, 200 μM and 200 M, respectively. The absorbance is measured on a microplate reader with 405 nm (A₄₀₅) in a kinetic mode every 30 second for at least 30 minutes at RT. The test compounds are screened at a concentration of 0.1 M or in an 8 point half log dilution series with a starting concentration of 10 M. The compound, (2R,4S)-N-((S)-1-(((1H-pyrrolo[3,2-c]pyridin-2-yl)methyl)amino)-1-oxopropan-2-yl)-4-phenylpiperidine-2-carboxamide is included in each assay in an 8 point half log dilution series with a starting concentration of 1 μM.

### OTHER EMBODIMENTS

This specification has been described with reference to various specific embodiments. However, one of ordinary skill in the art appreciates that various modifications and changes can be made without departing from the scope of the invention as set forth in the claims. Accordingly, the specification is to be regarded in an illustrative rather than a restrictive sense, and all such modifications are intended to be included within the scope of the claims.

We claim:

1. A compound of Formula (I): 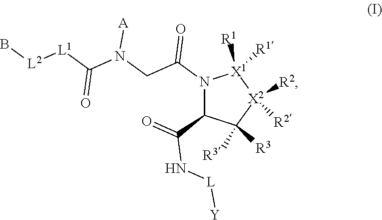
or a pharmaceutically acceptable salt thereof,
wherein
X¹ is C or N;
X² is C, S, or Si;
each of R¹, R^{1′}, R², R^{2′}, R³, and R^{3′} is independently selected from H, halo, hydroxy, optionally substituted C₁-C₆ alkyl, optionally substituted C₃-C₈ cycloalkyl, optionally substituted C₆-C₁₄ aryl, optionally substituted 5-to 10-membered heterocycle, or optionally substituted 5-to 10-membered heteroaryl; optionally substituted C₁-C₆ alkoxy; optionally substituted C₆-C₁₄ aryloxy, (optionally substituted 5-to 10-membered heteroaryl)oxy; SO₂R^{a}, wherein R^{a} is H, C₁-C₆ alkyl, or C₃-C₈ cycloalkyl; S(O)(NH)R^{b}, wherein R^{b} is H or C₁-C₆ alkyl, wherein, when X¹ is N, R⁸ or R¹ is absent and when X² is S, R² and R^{2′} are absent; or
R¹ and R², together with the atoms to which each is attached, form optionally substituted C₃-C₈ cycloalkyl; or
R¹ and R² combine to form a double bond;
R¹ and R^{2′}, together with the atoms to which each is attached, form optionally substituted C₃-C₈ cycloalkyl; or
R² and R^{2′}, together with the atom to which they are attached, form optionally substituted 4-or 5-membered spirocyclic heterocycle; or
R² and R^{2′}, together with the atom to which they are attached, form optionally substituted C₃-C₈ spirocyclic cycloalkyl; or
R² and R^{2′} combine to form=C(R^{c})₂, wherein each R^{c} is independently H or halo;
Y is: 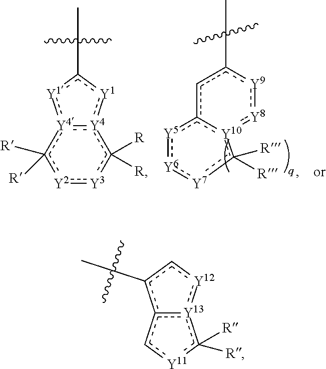
wherein
Y¹ is O, S, NR^{d}, wherein each R^{d} is independently absent, H, or C₁-C₆ alkyl;
Y^{1′} is O, S, NR^{d}, or C(R^{d})₂;
each of Y² and Y³ is independently NR^{e} or C(R^{e})₂, wherein each R^{e} is independently absent; H; optionally substituted C₁-C₆ alkyl; halo; or N(R^{g})₂, wherein each R^{g} is independently H or C₁-C₆ alkyl; or both R^{e} combine to form oxo;
each of Y⁴, Y^{4′}, Y¹⁰, and Y¹³ is independently CR^{e} or N;
each of Y⁵, Y⁶, and Y⁷ is independently O, S, NR^{f} or C(R^{f})₂, wherein each R^{f} is independently absent; H;
optionally substituted C₁-C₆ alkyl; halo; or N(R^{g})₂; or both R^{f} combine to form oxo;
each of YR and Y is independently C(R^{f})₂ or NR^{f};
each of Y¹¹ and Y¹² is independently NR^{e}, C(R^{e})₂, S, or O;
each [symbol] is independently a single bond or a double bond:
each of R, R′, R″, and R′″ is independently absent, H, optionally substituted C₁-C₆ alkyl, halo, or N(R^{g})₂; or
both R combine to form oxo; or
both R¹ combine to form oxo; and
q is 0 or 1;
L is selected from a bond, 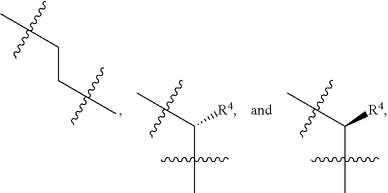
wherein R⁴ is H, CH₃, CF₃, or CH₂OH;
L¹ is a bond or optionally substituted C₁-C₆ alkylene;
L² is a bond or O;
B is optionally substituted C₆-C₁₄ aryl; optionally substituted C₃-C₁₄ carbocyclyl; optionally substituted 5-to 14-membered heterocyclyl; or optionally substituted 5-to 10-membered heteroaryl; and
A is H or C₁-C₆ alkyl; or
A is optionally substituted C₁-C₂ alkylene bound to a ring atom in B.

2. The compound of claim 1, wherein Y is

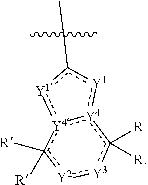

3. The compound of claim 2, wherein Y is

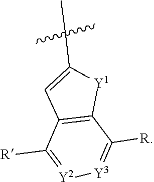

4. The compound of claim 3, wherein Y² is N.

5. The compound of claim 3, wherein Y² is CH or CNH₂.

6. The compound of any one of claims 3-5, wherein Y³ is N.

7. The compound of any one of claims 3-5, wherein Y³ is CR^{e}.

8. The compound of claim 7, wherein Y³ is CH or CNH₂.

9. The compound of claim 2, wherein Y is

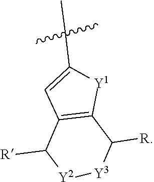

10. The compound of claim 9, wherein Y² is NH or NCH₃.

11. The compound of claim 10, wherein Y² is CH₂, CHCH₃, or C(O).

12. The compound of any one of claims 9-11, wherein Y³ is NH or NCH₃.

13. The compound of any one of claims 9-11, wherein Y³ is CH₂, CHCH₃, or C(O).

14. The compound of any one of claims 2-13, wherein Y is NH or NCH₃.

15. The compound of any one of claims 2-13, wherein Y¹ is O or S.

16. The compound of claim 2, wherein Y is

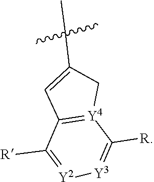

17. The compound of claim 16, wherein Y⁴ is N.

18. The compound of claim 16 or 17, wherein Y² is N.

19. The compound of claim 16 or 17, wherein Y² is CH.

20. The compound of any one of claims 16-19, wherein Y³ is N.

21. The compound of any one of claims 16-19, wherein Y³ is CH.

22. The compound of any one of claims 2-21, wherein R is H, CH₃, F, or Cl.

23. The compound of any one of claims 2-22, wherein R′ is H, CH₃, or NH₂.

24. The compound of claim 2, wherein Y is

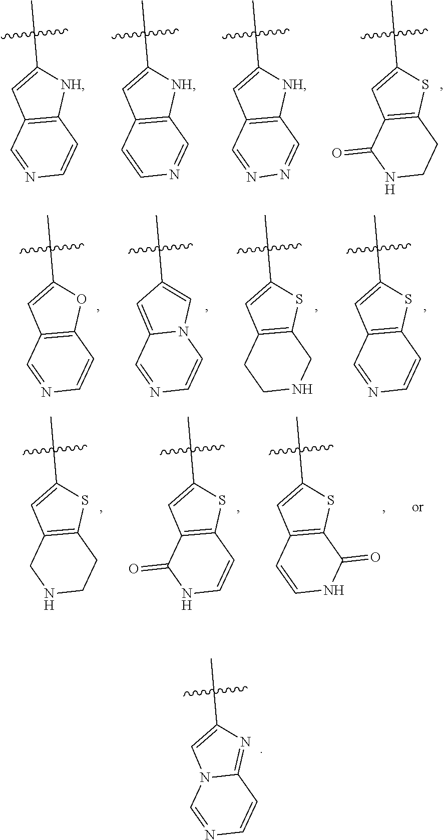

25. The compound of claim 1, wherein Y is

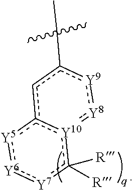

26. The compound of claim 25, wherein Y is

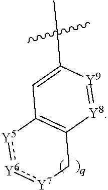

27. The compound of claim 26, wherein Y is

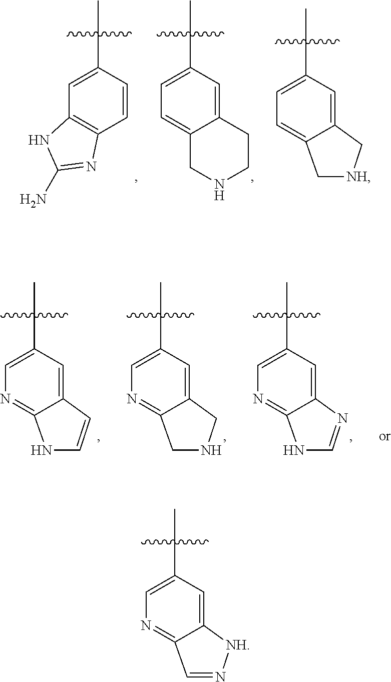

28. The compound of claim 1, wherein Y is

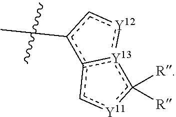

29. The compound of claim 28, wherein Y is

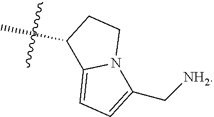

30. The compound of any one of claims 1-29, wherein B is optionally substituted C₆-C₁₄ aryl or optionally substituted 5-to 10-membered heteroaryl.

31. The compound of claim 30, wherein the compound is a compound of Formula (III): 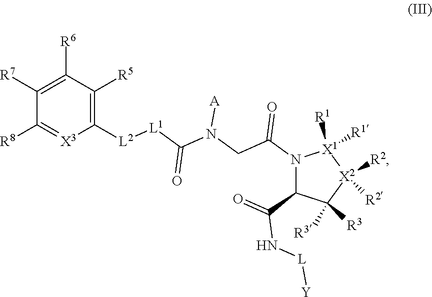
or a pharmaceutically acceptable salt thereof, wherein
X³ is CR⁹ or N;
each of R⁵, R⁶, and R⁹ is independently selected from H, halo, CN, SF₅, optionally substituted C₁-C₆ alkyl, optionally substituted C₁-C₆ alkoxy, S(O)(NH)CH₃, S(O)₂CH₃, and S(O)(NCN)CH₃; and
each of R⁷ and R⁸ is independently H, halo, CN, SF₅, optionally substituted C₁-C₆ alkyl, optionally substituted C₁-C₆ alkoxy, optionally substituted amino, S(O)(NH)CH₃, S(O)₂CH₃, S(O)(NCN)CH₃, optionally substituted C₃-C₈ cycloalkyl, optionally substituted C₃-C₈ cycloalkyloxy, optionally substituted C₆-C₁₄ aryloxy, optionally substituted C₆-C₁₄ aryl, optionally substituted 5-to 10-membered heterocyclyl, optionally substituted 5-to 10-membered heteroaryl, optionally substituted (5-to 10-membered heteroaryl)oxy, or optionally substituted (4-to 10-membered heterocyclyl)oxy, provided that no more than one of R⁷ and R⁸ is optionally substituted C₆-C₁₄ aryloxy, optionally substituted C₆-C₁₄ aryl, optionally substituted 5-to 10-membered heteroaryl, optionally substituted (5-to 10-membered heteroaryl)oxy, or optionally substituted (4-to 10-membered heterocyclyl)oxy; or
R⁷ and R⁸, together with the atoms to which each is attached, form optionally substituted 5-to 6-membered heterocyclyl, optionally substituted 5-to 10-membered heteroaryl, or optionally substituted C₆-C₁₄ aryl; or
R⁵ and A combine to form optionally substituted C₁-C₂ alkylene; or
R⁶ and R⁹ combine to form (C₂-C₆alkylene)(C₆-C₁₄ arylene)(C₂-C₆alkylene), and each of R⁵, R⁷, and R⁸ is H.

32. The compound of claim 31, wherein X³ is CH or CF.

33. The compound of claim 31, wherein X³ is N.

34. The compound of any one of claims 31-33, wherein R⁷ is optionally substituted C₆-C₁₄ aryl.

35. The compound of claim 34, wherein R⁷ is optionally substituted phenyl.

36. The compound of claim 35, wherein R⁷ is

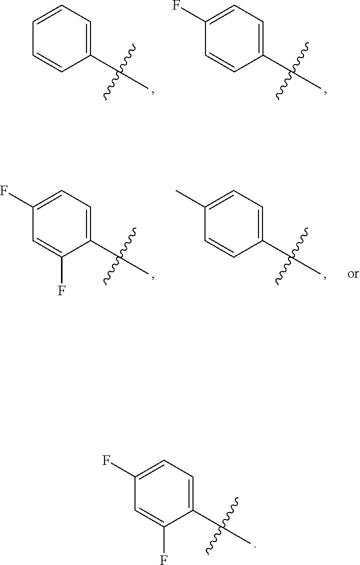

37. The compound of any one of claims 31-33, wherein R⁷ is optionally substituted 5-to 10-membered heteroaryl.

38. The compound of claim 37, wherein R⁷ is

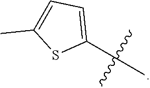 [SMILES: *c1ccc(C)s1]

39. The compound of any one of claims 31-33, wherein R⁷ is optionally substituted C₃-C₈ cycloalkyloxy.

40. The compound of claim 39, wherein R⁷ is

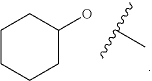

41. The compound of claim 40, wherein R⁷ is Br or Cl.

42. The compound of any one of claims 31-33, wherein R⁷ is H.

43. The compound of any one of claims 31-42, wherein R⁸ is H or halo.

44. The compound of claim 43, wherein R⁸ is optionally substituted phenyl.

45. The compound of claim 44, wherein R⁸ is

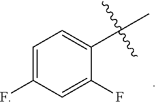 [SMILES: *c1ccc(F)cc1F]

46. The compound of any one of claims 31-33, wherein R⁸ is optionally substituted phenoxy.

47. The compound of claim 46, wherein R⁸ is

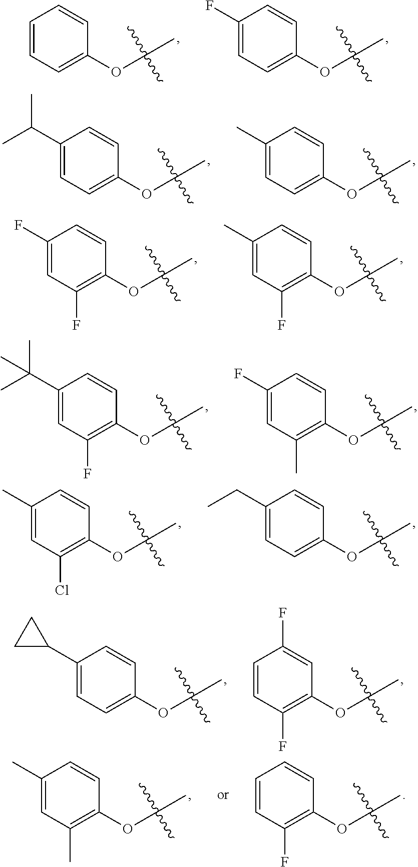

48. The compound of any one of claims 31-33, wherein R⁸ is (optionally substituted 5-to 10-membered heteroaryl)oxy.

49. The compound of claim 48, wherein R⁸ is

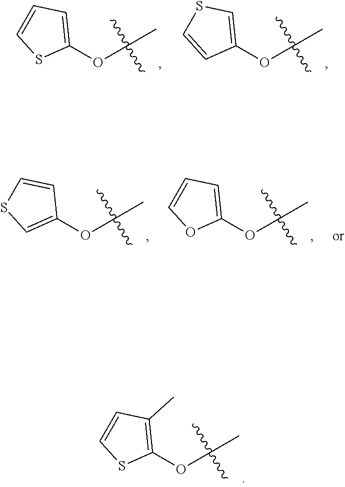

50. The compound of any one of claims 31-33, wherein R⁸ is optionally substituted 5-to 10-membered heteroaryl.

51. The compound of claim 50, wherein R⁸ is

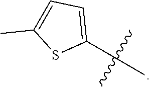 [SMILES: *c1ccc(C)s1]

52. The compound of any one of claims 31-33, wherein R⁸ is optionally substituted C₁-C₆ alkoxy.

53. The compound of claim 52, wherein R⁸ is

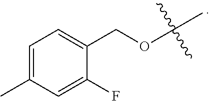 [SMILES: *OCc1ccc(C)cc1F]

54. The compound of any one of claims 31-33, wherein R⁸ is optionally substituted 5-to 10-membered heterocyclyl.

55. The compound of claim 54, wherein R⁸ is

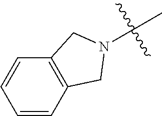 [SMILES: *N1Cc2ccccc2C1]

56. The compound of any one of claims 44-55, wherein R⁷ is H or C₁-C₆ alkyl.

57. The compound of any one of claims 31-33, wherein R⁷ and R⁸, together with the atoms to which each is attached, form optionally substituted 5-to 6-membered heterocyclyl.

58. The compound of claim 57, wherein R⁷ and R⁸, together with the atoms to which each is attached, form

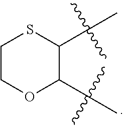 [SMILES: *C1OCCSC1*]

59. The compound of any one of claims 31-58, wherein R⁶ is H, halo, or optionally substituted C₁-C₆ alkyl.

60. The compound of any one of claims 31-59, wherein R⁵ is H, halo, or optionally substituted C₁-C₆ alkoxy.

61. The compound of claim 31-59, wherein R⁵ and A combine to form —CH₂—.

62. The compound of claim 30, wherein the compound is a compound of Formula (IV): 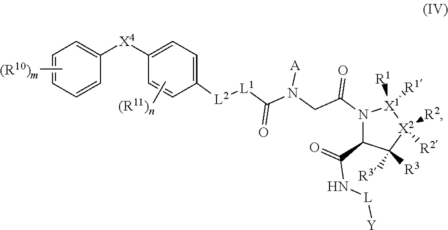
or a pharmaceutically acceptable salt thereof, wherein
X⁴ is O; C(R^{h})₂, wherein each R^{h} is independently hydrogen, halo, or optionally substituted C₁-C₆ alkyl, or both R^{h} combine to form oxo; S(O)₂, or NR^{h};
m is selected from 0, 1, 2, 3, 4, and 5;
n is selected from 0, 1, 2, 3, and 4; and
each R¹⁰ and R¹¹ is independently halo, CN, optionally substituted C₁-C₆ alkyl, optionally substituted C₁-C₆ alkoxy, or optionally substituted C₃-C₈ cycloalkyl.

63. The compound of claim 62, wherein X⁴ is O.

64. The compound of claim 63, wherein B is

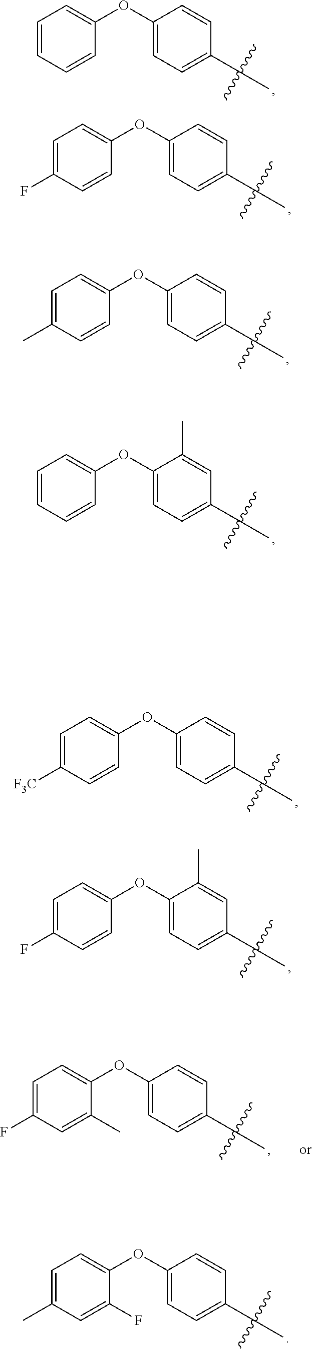

65. The compound of claim 62, wherein X⁴ is C(O).

66. The compound of claim 65, wherein B is

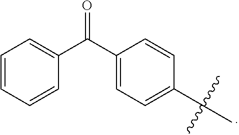 [SMILES: *c1ccc(C(=O)c2ccccc2)cc1]

67. The compound of claim 30, wherein the compound is a compound of Formula (V): 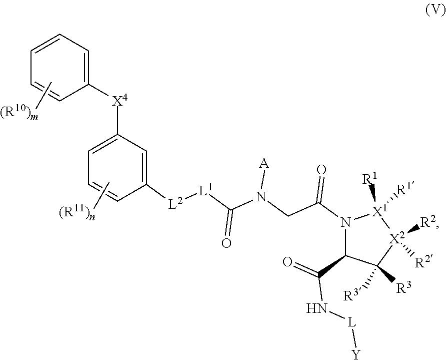
or a pharmaceutically acceptable salt thereof, wherein
X⁴ is O; C(R^{h})₂, wherein each R^{h} is independently hydrogen, halo, or optionally substituted C₁-C₆ alkyl, or both R^{h} combine to form oxo; S(O)₂, or NR^{h};
m is selected from 0, 1, 2, 3, 4, and 5;
n is selected from 0, 1, 2, 3, and 4; and
each R¹⁰ and R¹¹ is independently halo, CN, optionally substituted C₁-C₆ alkyl, optionally substituted C₁-C₆ alkoxy, or optionally substituted C₃-C₈ cycloalkyl.

68. The compound of claim 67, wherein X⁴ is O.

69. The compound of claim 68, wherein B is

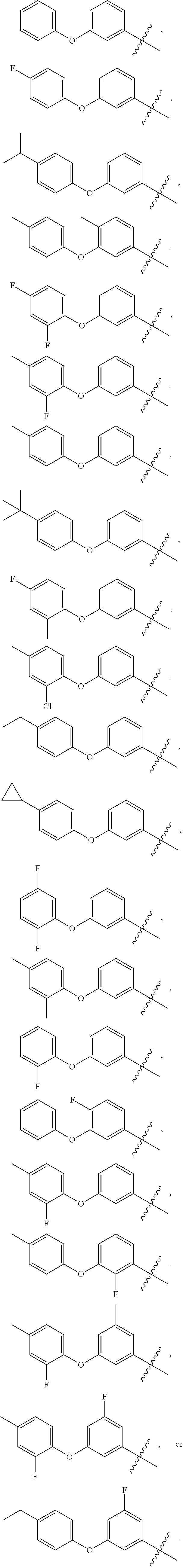

70. The compound of claim 67, wherein X⁴ is CH₂.

71. The compound of claim 70, wherein B is

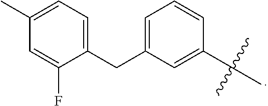 [SMILES: *c1cccc(Cc2ccc(C)cc2F)c1]

72. The compound of claim 67, wherein X⁴ is NH.

73. The compound of claim 72, wherein B is

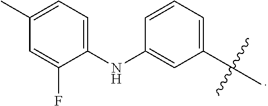 [SMILES: *c1cccc(Nc2ccc(C)cc2F)c1]

74. The compound of any one of claims 1-29, wherein B is optionally substituted C₃-C₁₄ carbocyclyl or optionally substituted 5-to 14-membered heterocyclyl.

75. The compound of claim 74, wherein the compound is a compound of formula (VI): 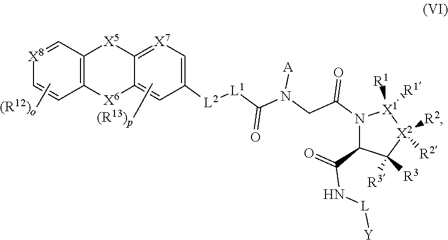
or a pharmaceutically acceptable salt thereof, wherein
each of X⁵ and X⁶ is independently a bond; O; S; C(Rⁱ)₂, wherein each Rⁱ is independently H, OH, halo, optionally substituted C₁-C₆ alkyl, or optionally substituted C₁-C₆ alkoxy, or both Rⁱ combine to form oxo; NR^{j}, wherein R^{j} is H or C₁-C₆ alkyl; or SO₂;
X⁷ is CH, CR¹³, or N;
X⁸ is CH, CR¹², or N;
o is selected from 0, 1, 2, and 3;
p is selected from 0, 1, and 2; and
each R¹² and R¹³ is independently halo, optionally substituted C₁-C₆ alkyl, optionally substituted C₁-C₆ alkoxy, or optionally substituted C₃-C₈ cycloalkyl.

76. The compound of claim 74, wherein the compound is a compound of formula (VII): 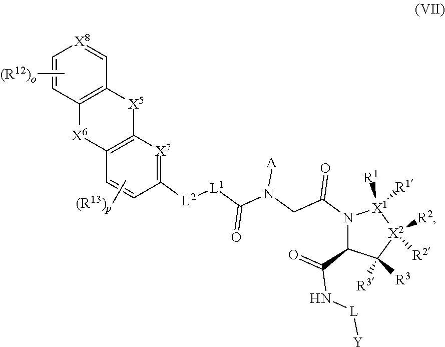
or a pharmaceutically acceptable salt thereof, wherein
each of X⁵ and X⁶ is independently a bond; O; S; C(Rⁱ)₂, wherein each Rⁱ is independently H, OH, halo, optionally substituted C₁-C₆ alkyl, or optionally substituted C₁-C₆ alkoxy, or both Rⁱ combine to form oxo; NR^{j}, wherein R^{j} is H or C₁-C₆ alkyl; or SO₂;
X⁷ is CH, CR¹³, or N;
X⁸ is CH, CR¹², or N;
o is selected from 0, 1, 2, and 3;
p is selected from 0, 1, and 2; and
each R¹² and R¹³ is independently halo, optionally substituted C₁-C₆ alkyl, optionally substituted C₁-C₆ alkoxy, or optionally substituted C₃-C₈ cycloalkyl.

77. The compound of claim 75 or 76, wherein X⁵ is CF₂, CHCH₃, C(CH₃)₂, or C(CH₃)(OH).

78. The compound of claim 75 or 76, wherein X⁵ is a bond.

79. The compound of claim 75 or 76, wherein X⁵ is O.

80. The compound of claim 75 or 76, wherein X⁵ is S.

81. The compound of claim 75 or 76, wherein X⁵ is NH.

82. The compound of any one of claims 75-81, wherein X⁶ is a bond.

83. The compound of any one of claims 75-81, wherein X⁶ is O.

84. The compound of any one of claims 75-81, wherein X⁶ is S.

85. The compound of any one of claims 75-81, wherein X⁶ is CF₂, CHCH₃, C(CH₃)₂, or C(CH₃)(OH).

86. The compound of any one of claims 75-81, wherein X⁶ is NH.

87. The compound of any one of claims 75-86, wherein X⁷ is CH or N.

88. The compound of any one of claims 75-86, wherein X⁸ is CH or N.

89. The compound of claim 75, wherein B is

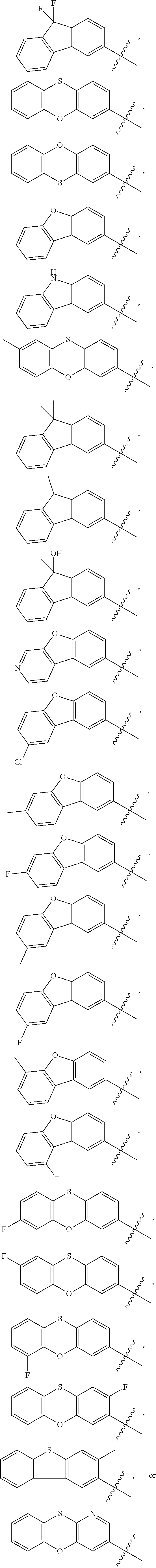

90. The compound of claim 76, wherein B is

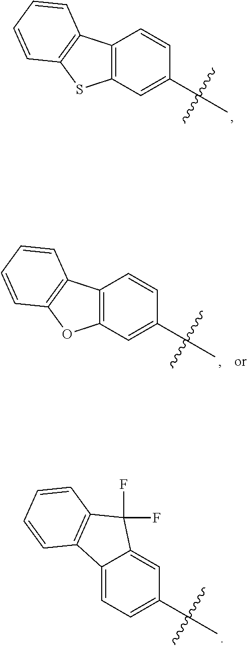 [SMILES: *c1ccc2c(c1)sc1ccccc12 ; *c1ccc2c(c1)oc1ccccc12 ; *c1ccc2c(c1)C(F)(F)c1ccccc1-2]

91. The compound of claim 1-29, wherein B is optionally substituted 5-to 10-membered heteroaryl.

92. The compound of claim 91, wherein B is

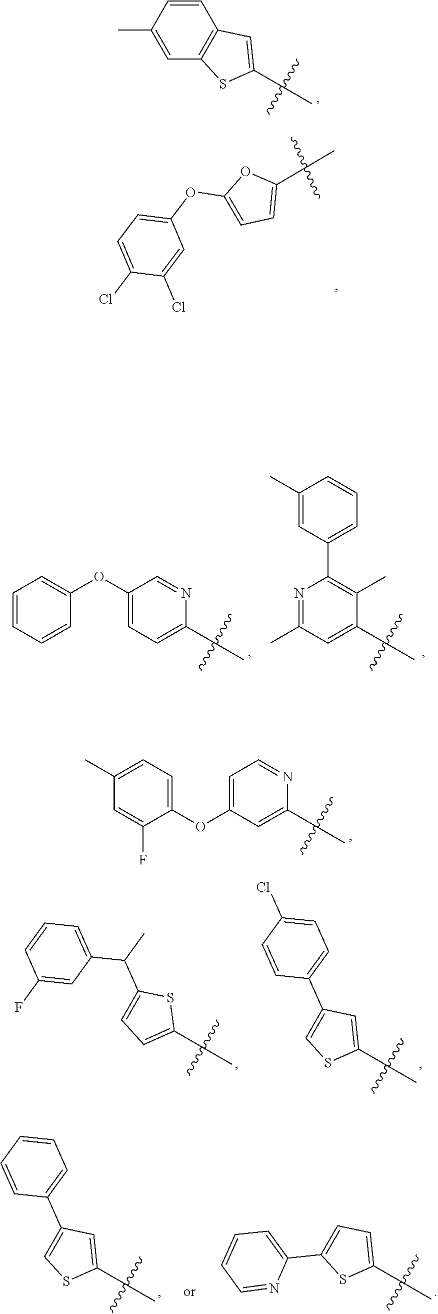

93. The compound of any one of claims 1-92, wherein X¹ is C and X² is C.

94. The compound of claim 1-93, wherein R¹ and R², together with the atoms to which each is attached, combine to form cyclopropyl.

95. The compound of claim 1-93, wherein R^{1′} and R^{2′}, together with the atoms to which each is attached, combine to form cyclopropyl.

96. The compound of any one of claims 1-92, wherein X¹ is N and X² is C.

97. The compound of claim 93 or 96, wherein R¹ and R² combine to form a double bond.

98. The compound of claim 93 or 96, wherein R² and R^{2′}, together with the atom to which they are attached, form 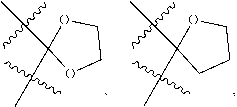 [SMILES: *C1(*)OCCO1 ; *C1(*)CCCO1]
spirocyclic cyclopropyl, or spirocyclic cyclopentyl.

99. The compound of any one of claims 1-93, 95, and 96, wherein R² is H.

100. The compound of any one of claims 1-93, 95, and 96, wherein R² is optionally substituted C₁-C₆ allyl.

101. The compound of claim 100, wherein R² is CH₃, CH₂F, CH₂O CH₃, CH₂O(CH₂)₄NH₂, CH₂O(CH₂)₄NHC(O)CH₃,

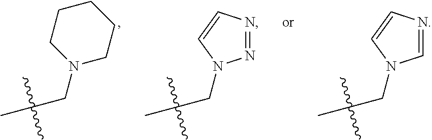 [SMILES: *CN1CCCCC1 ; *Cn1ccnn1 ; *Cn1ccnc1]

102. The compound of any one of claims 1-93, 95, and 96, wherein R² is optionally substituted optionally substituted 5-to 10-membered heteroaryl or optionally substituted 5-to 10-membered heterocycle.

103. The compound of claim 102, wherein R² is

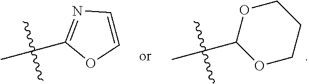 [SMILES: *c1ncco1 ; *C1OCCCO1]

104. The compound of any one of claims 1-94, 96, 97, and 99-103, wherein R^{2′} is H.

105. The compound of any one of claims 1-94, 96, 97, and 99-103, wherein R², is F.

106. The compound of any one of claims 1-94, 96, 97, and 99-103, wherein R^{2′} is optionally substituted C₁-C₆ alkyl.

107. The compound of claim 106, wherein R^{2′} is CH₃, CF₃, CH₂OH, CH₂OCH₃, CH₂O(CH₂)₄NH₂, CH₂O(CH₂)₅COOH,

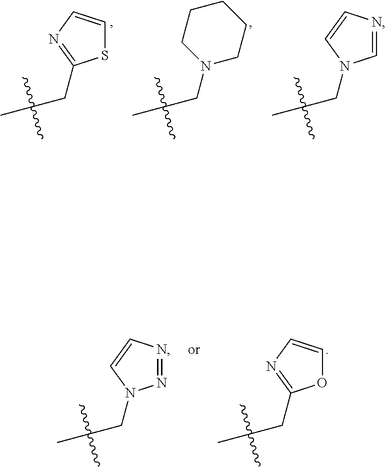 [SMILES: *Cc1nccs1 ; *CN1CCCCC1 ; *Cn1ccnc1 ; *Cn1ccnn1 ; *Cc1ncco1]

108. The compound of any one of claims 1-94, 96, 97, and 99-103, wherein R^{2′} is optionally substituted optionally substituted 5-to 10-membered heteroaryl or optionally substituted 5-to 10-membered heterocycle.

109. The compound of claim 108, wherein R² is

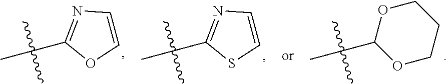 [SMILES: *c1ncco1 ; *c1nccs1 ; *C1OCCCO1]

110. The compound of any one of claims 1-94, 96, 97, and 99-103, wherein R^{2′} is optionally substituted C₁-C₆ alkoxy.

111. The compound of claim 110, wherein R^{2′} is OCH₃, OCHF₂, or OCF₃.

112. The compound of any one of claims 1-94, 96, 97, and 99-103, wherein R^{2′} is SO₂CH₃, SO₂CH(CH₃)₂, or

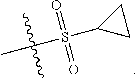 [SMILES: *S(=O)(=O)C1CC1]

113. The compound of any one of claims 1-92, wherein X¹ is C and X² is Si.

114. The compound of claim 146, wherein R² and R^{2′} are each optionally substituted C₁-C₆ alkyl.

115. The compound of any one of claims 1-92, wherein X¹ is C and X² is S.

116. The compound of any one of claims 1-93, 95, 96, and 98-115, wherein R⁸ is H.

117. The compound of any one of claims 1-93, 95, 96, and 98-115, wherein R¹ is CH₃.

118. The compound of any one of claims 1-95 and 96-117, wherein R^{1′} is H.

119. The compound of any one of claims 1-118, wherein R³ is H or hydroxy.

120. The compound of any one of claims 1-118, wherein R^{3′} is H.

121. The compound of any one of claims 1-120, wherein L is

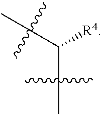

122. The compound of claim 121, wherein R⁴ is H or CH₃.

123. The compound of any one of claims 1-122, wherein L¹ is a bond.

124. The compound of any one of claims 1-122, wherein L¹ is —CH₂— or —(CH₂)₃—.

125. The compound of any one of claims 1-124, wherein L² is a bond.

126. The compound of any one of claims 1-124, wherein L² is O.

127. The compound of any one of claims 1-60 and 62-126, wherein A is H.

128. A compound of Table 1, or a pharmaceutically acceptable salt thereof.

129. The compound of any one of claims 1-128, having complement C₁ esterase (C1s) inhibiting activity.

130. A pharmaceutical composition comprising the compound or pharmaceutically acceptable salt of any one of claims 1-129 and a pharmaceutically acceptable excipient.

131. A method of treating C1s mediated disorder, comprising administering to a subject in need thereof a therapeutically effective amount of the compound or pharmaceutically acceptable salt of any one of claims 1-129.

132. The method of claim 131, wherein the subject is a human.

133. The method of claim 131 or 132, wherein the disorder is acute antibody-mediated rejection, amyotrophic lateral sclerosis, autoimmune blistering disease, bullous pemphigoid, chronic inflammatory demyelinating polyneuropathy, geographic atrophy, Guillain-Barré Syndrome, Huntington's Disease, immune thrombocytopenia purpura, lupus nephritis, multifocal motor neuropathy, rheumatoid arthritis, traumatic brain injury, and warm autoimmune hemolytic anemia.
